(19) United States
(12) Patent Application Publication
(10) Pub. No.: US 2026/0053834 A1
(43) Pub. Date: Feb. 26, 2026

(54) COVALENT MODIFIERS OF AKT1 AND USES THEREOF

(71) Applicant: Terremoto Biosciences, Inc., South San Francisco, CA (US)

(72) Inventors: Hang Chu, San Mateo, CA (US); Solomon H. Reisberg, Contra Costa, CA (US); Adam Zajdlik, San Francisco, CA (US); Kin S. Yang, San Mateo, CA (US); Jordan D. Carelli, San Francisco, CA (US); Peter A. Thompson, Kirkland, WA (US)

(21) Appl. No.: 19/305,122

(22) Filed: Aug. 20, 2025

Related U.S. Application Data
(63) Continuation of application No. PCT/US2024/017162, filed on Feb. 23, 2024.
(60) Provisional application No. 63/486,884, filed on Feb. 24, 2023.
(60) Provisional application No. 63/506,224, filed on Jun. 5, 2023.
(60) Provisional application No. 63/618,164, filed on Jan. 5, 2024.

(51) Int. Cl. A61K 31/69; A61K 31/444; A61K 31/4725; A61K 31/496; A61K 31/497; A61K 31/501; A61K 31/506; A61K 31/517; A61K 31/5377; A61K 31/5383; A61P 35/00; C07B 59/00; C07D 471/04; C07D 519/00; C07F 5/02

(52) U.S. Cl. CPC A61K 31/69; A61K 31/444; A61K 31/4725; A61K 31/496; A61K 31/497; A61K 31/501; A61K 31/506; A61K 31/517; A61K 31/5377; A61K 31/5383; A61P 35/00; C07B 59/002; C07D 471/04; C07D 519/00; C07F 5/025

(57) ABSTRACT

Provided herein are covalent modifiers of AKT1 of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), and pharmaceutical compositions thereof. In some embodiments, the present disclosure provides methods of modulating AKT1 using a compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), and pharmaceutical compositions thereof.

### CROSS REFERENCE TO RELATED APPLICATIONS

This application is a continuation application of International Patent Application No. PCT/US2024/017162, filed Feb. 23, 2024, which claims the benefit of U.S. Provisional Application No. 63/486,884, filed Feb. 24, 2023, U.S. Provisional Application No. 63/506,224, filed Jun. 5, 2023, and U.S. Provisional Application No. 63/618,164, filed Jan. 5, 2024, the entirety of each is incorporated herein by reference.

### BACKGROUND OF THE INVENTION

The AKT or Protein Kinase B (PKB) family of serine/threonine protein kinases is comprised of 3 highly homologous members, AKT1, AKT2 and AKT3. The family of AKT proteins are involved in signal transduction pathways that regulate cellular processes including apoptosis, proliferation, differentiation and metabolism. The AKT1 pathway is the most frequently dysregulated signaling pathways in human cancers. Enhanced activation of all the isoforms can be implicated in tumor development and progression, and has been demonstrated in breast, ovarian, pancreatic, and prostate cancers among others (Song et al., 2019). In cancer cells, AKT1 is involved in proliferation and growth, promoting tumor initiation and suppressing apoptosis, whereas AKT2 regulates cytoskeleton dynamics, favoring local tissue invasion and metastasis. The role of AKT3 hyperactivation in cancer is hypothesized to be involved with possible stimulation of cell proliferation (Hinz et al., Cell Commun Signal 2019, 17(1), 154; Pascual et al., Ann. Oncol. 2019, 30(7), 1051-1060). Expression of these AKT family members is altered in many human malignant carcinomas including gastric, breast, prostate, ovarian and pancreatic. AKT family members are rarely mutated however, the most common mutation is AKT1 E17K which has been reported in 6-8% of breast cancers, 2-6% of colorectal cancers, and in 6% of meningiomas, in human (Yu et al., PLoS One 2015, 10 (10), No. e0140479). Thus, there is a need to develop new treatments for the modulation of AKT1 and mutants thereof.

### SUMMARY OF THE INVENTION

In one aspect, the present disclosure provides a compound represented by the structure of Formula (A):

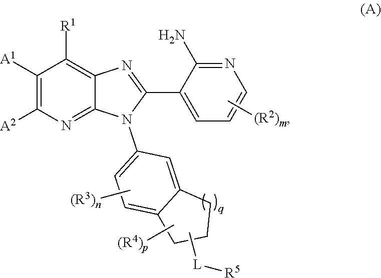

or a pharmaceutically acceptable salt thereof, wherein:
R¹ is selected from:
   hydrogen, halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN;
   C₁₋₆ alkyl, C₂₋₆ alkenyl, and C₂₋₆ alkynyl, any of which is optionally substituted with one or more substituents independently selected from: halogen —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN; and
   3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, ═O, —NO₂, —CN, C₁₋₆ alkyl, and C₁₋₆ haloalkyl;
A¹ and A² are each independently selected from:
   hydrogen, halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
   C₁₋₆ alkyl, C₂₋₆ alkenyl, and C₂₋₆ alkynyl, any of which is optionally substituted with one or more substituents independently selected from: halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   5- to 10-membered heterocycle and C₃₋₁₀ carbocycle, any of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
      C₁₋₆ alkyl, C₂₋₆ alkenyl, and C₂₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
      C₃₋₁₀ carbocycle and 3- to 10-membered heterocycle; wherein the C₃₋₁₀ carbocycle and 3- to 10-membered heterocycle are each optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
         C₁₋₆ alkyl, C₂₋₆ alkenyl, and C₂₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN;
q is selected from 1, 2, and 3;
m and n are each independently selected from 0, 1, 2, and 3;
R² is independently selected at each instance from halogen, C₁₋₄ alkyl, C₁₋₄ haloalkyl, —OR¹², —SR¹², —N(R¹²)₂, —NO₂, and —CN;
R³ is independently selected at each instance from:
   halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
   C₁₋₆ alkyl optionally substituted with one or more substituents independently selected from: halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN;
p is selected from 0, 1, 2, 3, 4, and 5;
R⁴ is independently selected at each instance from:
   halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
   C₁₋₆ alkyl, C₂₋₆ alkenyl, C₂₋₆ alkynyl, any one of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
two R⁴ attached to the same atom are taken together to form a group selected from:
   ═O, ═S, and ═N(R¹⁴); or
two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from: halogen, C₁₋₄ alkyl, C₁₋₄ haloalkyl, —OR¹⁴, —SR¹⁴, —N(R¹⁴)₂, —NO₂, and —CN;
L is a bond or represented by -L¹-L²-L³-L⁴-, wherein L¹, L², L³, and L⁴ are each independently selected from (a) and (b):
   (a) —O—, —N(R¹⁵)—, —S—, —S(O)—, —S(O)₂—, —S(O)(NR¹⁵)—, —N(R¹¹)C(O)—, —N(R¹⁵)C(O)O—, —N(R¹¹)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
   (b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
      wherein L², L³, and L⁴ are each optionally absent;
      wherein no more than two of L¹, L², L³, and L⁴ are selected from (a) and the two selected are not adjacent;
R⁵ is selected from 3- to 12-membered heterocycle and C₃₋₁₂carbocycle, any of which is substituted by —C(O)H or —C(O)D, wherein R⁵ is further optionally substituted by one or more substituents independently selected from:
   halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN;
   C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
   4- to 6-membered heterocycle optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), —CN, C₁₋₆ alkyl, and C₁₋₆ haloalkyl;
R¹⁰, R¹¹, R¹², R¹³, R¹⁴, and R¹⁵ are each independently selected at each occurrence from:
   hydrogen, C₁₋₄ alkyl, C₃₋₈ carbocycle, 3- to 8-membered heterocycle, and C₁₋₄ haloalkyl;
R¹⁶ is independently selected at each occurrence from:
   hydrogen;
   C₁₋₄ alkyl, wherein the C₁₋₄ alkyl is optionally substituted with one or more substituents independently selected from:
      halogen, —OR²⁰, —SR²⁰, —N(R²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
      C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
         C₃₋₈ carbocycle and 3- to 8-membered heterocycle, either of which is optionally substituted with one or more substituents independently selected from:
      halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —NO₂, and —CN; and
R²⁰ is independently selected at each occurrence from hydrogen, C₁₋₄ alkyl, C₁₋₄ haloalkyl, C₃₋₈ carbocycle, and 3- to 8-membered heterocycle.

In one aspect, the present disclosure provides a pharmaceutical composition comprising a pharmaceutically acceptable excipient and a compound of Formula (A), (A-1), or (I), or a pharmaceutically acceptable salt thereof.

In one aspect, the present disclosure provides a method of modulating activity of a mutant AKT1 comprising, administering to a subject in need thereof a compound of Formula (A), (A-1), or (I), or a pharmaceutically acceptable salt thereof, or a pharmaceutical composition of the present disclosure comprising a pharmaceutically acceptable excipient and a compound of Formula (A), (A-1), or (I), or a pharmaceutically acceptable salt thereof. In some embodiments, the mutant AKT1 is AKT1 E17K.

In one aspect, the present disclosure provides a method of selectively modulating activity of a mutant AKT1 over a wild type AKT comprising administering to a subject in need thereof a compound of Formula (A), (A-1), or (I), or pharmaceutically acceptable salt thereof, or a pharmaceutical composition of the present disclosure comprising a pharmaceutically acceptable excipient and a compound of Formula (A), (A-1), or (I), or a pharmaceutically acceptable salt thereof, wherein the wild type AKT is selected from wild type AKT1 and wild type AKT2. In some embodiments, the mutant AKT1 is AKT1 E17K.

In one aspect, the present disclosure provides a method of treating cancer in a subject in need thereof, the method comprising administering to the subject a compound of Formula (A), (A-1), or (I), or pharmaceutically acceptable salt thereof, or a pharmaceutical composition of the present disclosure comprising a pharmaceutically acceptable excipient and a compound of Formula (A), (A-1), or (I), or a pharmaceutically acceptable salt thereof. In some embodiments, the cancer is selected from breast cancer, colorectal cancer, and meningioma. In some embodiments, the administration modulates activity of a mutant AKT1. In some embodiments, the mutant AKT1 is AKT1 E17K.

### INCORPORATION BY REFERENCE

All publications, patents, and patent applications mentioned in this specification are herein incorporated by reference for the specific purposes identified herein.

### DETAILED DESCRIPTION OF THE INVENTION

The AKT or Protein Kinase B (PKB) family of serine/threonine protein kinases regulate a myriad of key cellular functions, including apoptosis, proliferation, differentiation and metabolism. The AKT family is comprised of 3 highly homologous members, AKT1, AKT2 and AKT3, and each member possesses a unique tissue distribution and may perform a unique set of biological functions. Aberrant expression and/or activation of all AKT isoforms has been implicated in tumor development, including breast, ovarian, pancreatic, and prostate cancers among others.

Inhibitors of AKT proteins have been developed for the treatment of cancer, including the two major classes of small-molecule AKT inhibitors being investigated in the clinic: allosteric and ATP-competitive inhibitors. First, allosteric inhibitors (such as miransertib (ARQ 092) and MK-2206) interfere with PH-domain mediated membrane recruitment (the first step in AKT activation) and inhibit AKT kinase activation and AKT phosphorylation. Second, ATP-competitive inhibitors of AKT (such as ipatasertib and capivasertib) bind to the active kinase, in which the PH-domain has shifted from the kinase domain and exposed the ATP-binding pocket site, thus inhibiting ATP binding.

However, none of these examples covalently bind to the AKT protein, creating a covalent complex (e.g., a reversible covalent complex having a slow off-rate). Covalent inhibitors (in particular reversible covalent inhibitors) provide a number of advantages over non-covalent inhibitors, including but not limited to increased residence time on target, increased selectivity for particular isoforms, increased selectivity for mutant targets, and improved pharmacokinetics.

Provided herein are compounds for modulating (e.g., inhibiting) AKT1 function, as well as methods and compositions for using compounds of the present disclosure in the treatment of cancer. The present disclosure provides compounds having a reversible covalent electrophile that forms a covalent complex between an amine on the AKT1 protein and the electrophile on the compound. In some embodiments, the reversible covalent electrophile is an aldehyde (e.g., an aromatic aldehyde) that forms a reversible covalent imine bond between an amine (e.g., amine on a lysine sidechain) on the AKT1 protein. In some embodiments, the compounds selectively inhibit (e.g., 2×, 5×, 10×, 50×, 100×, etc.) an AKT1 protein over an AKT2 and/or AKT3 protein. In some embodiments, the compounds selectively inhibit (e.g., 2×, 5×, 10×, 50×, 100×, etc.) a mutant AKT1 (e.g., E17K AKT1) over a wild-type AKT1 protein.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an”, and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁₋₁₂ alkyl). The alkyl is attached to the remainder of the molecule through a single bond. An alkyl chain may be optionally substituted by one or more substituents such as those substituents described herein. In certain embodiments, an alkyl comprises one to twelve carbon atoms (i.e., C₁₋₁₂ alkyl). In certain embodiments, an alkyl comprises one to eight carbon atoms (i.e., C₁₋₈ alkyl). In other embodiments, an alkyl comprises one to five carbon atoms (i.e., C₁₋₅ alkyl). In other embodiments, an alkyl comprises one to four carbon atoms (i.e., C₁₋₄ alkyl). In other embodiments, an alkyl comprises one to three carbon atoms (i.e., C₁₋₃ alkyl). In other embodiments, an alkyl comprises one to two carbon atoms (i.e., C₁₋₂ alkyl). In other embodiments, an alkyl comprises one carbon atom (i.e., C₁ alkyl). In other embodiments, an alkyl comprises five to fifteen carbon atoms (i.e., C₅₋₁₅ alkyl). In other embodiments, an alkyl comprises five to eight carbon atoms (i.e., C₅₋₈ alkyl). In other embodiments, an alkyl comprises two to five carbon atoms (i.e., C₂₋₅ alkyl). In other embodiments, an alkyl comprises three to five carbon atoms (i.e., C₃₋₅ alkyl). For example, the alkyl group may be attached to the rest of the molecule by a single bond, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂₋₁₂ alkenyl). An alkenyl chain may be optionally substituted by one or more substituents such as those substituents described herein. In certain embodiments, an alkenyl comprises two to eight carbon atoms (i.e., C₂₋₈ alkenyl). In certain embodiments, an alkenyl comprises two to six carbon atoms (i.e., C₂₋₆ alkenyl). In other embodiments, an alkenyl comprises two to four carbon atoms (i.e., C₂₋₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂₋₁₂ alkynyl). An alkylnyl chain may be optionally substituted by one or more substituents such as those substituents described herein. In certain embodiments, an alkynyl comprises two to eight carbon atoms (i.e., C₂₋₈ alkynyl). In other embodiments, an alkynyl comprises two to six carbon atoms (i.e., C₂₋₆ alkynyl). In other embodiments, an alkynyl comprises two to four carbon atoms (i.e., C₂₋₄ alkynyl). The alkynyl is attached to the rest of the molecule by a single bond, for example, ethynyl, propynyl, butynyl, pentynyl, hexynyl, and the like.

“Alkylene” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, (methyl)ethylene, butylene, and the like. The alkylene chain is attached to the rest of the molecule through a single bond and to the radical group through a single bond. An alkylene chain may be optionally substituted by one or more substituents such as those substituents described herein. In certain embodiments, an alkylene comprises one to ten carbon atoms (i.e., C₁₋₁₀ alkylene). In certain embodiments, an alkylene comprises one to eight carbon atoms (i.e., C₁₋₈ alkylene). In other embodiments, an alkylene comprises one to five carbon atoms (i.e., C₁₋₅ alkylene). In other embodiments, an alkylene comprises one to four carbon atoms (i.e., C₁₋₄ alkylene). In other embodiments, an alkylene comprises one to three carbon atoms (i.e., C₁₋₃ alkylene). In other embodiments, an alkylene comprises one to two carbon atoms (i.e., C₁₋₂ alkylene). In other embodiments, an alkylene comprises one carbon atom (i.e., C₁ alkylene). In other embodiments, an alkylene comprises five to eight carbon atoms (i.e., C₅₋₈ alkylene). In other embodiments, an alkylene comprises two to five carbon atoms (i.e., C₂₋₅ alkylene). In other embodiments, an alkylene comprises three to five carbon atoms (i.e., C₃₋₅ alkylene).

“Alkenylene” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. An alkenylene chain may be optionally substituted by one or more substituents such as those substituents described herein. In certain embodiments, an alkenylene comprises two to ten carbon atoms (i.e., C₂₋₁₀ alkenylene). In certain embodiments, an alkenylene comprises two to eight carbon atoms (i.e., C₂₋₈ alkenylene). In other embodiments, an alkenylene comprises two to five carbon atoms (i.e., C₂₋₅ alkenylene). In other embodiments, an alkenylene comprises two to four carbon atoms (i.e., C₂₋₄ alkenylene). In other embodiments, an alkenylene comprises two to three carbon atoms (i.e., C₂₋₃ alkenylene). In other embodiments, an alkenylene comprises two carbon atoms (i.e., C₂ alkenylene). In other embodiments, an alkenylene comprises five to eight carbon atoms (i.e., C₅₋₈ alkenylene). In other embodiments, an alkenylene comprises three to five carbon atoms (i.e., C₃₋₅ alkenylene).

“Alkynylene” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. An alkynylene chain may be optionally substituted by one or more substituents such as those substituents described herein. In certain embodiments, an alkynylene comprises two to ten carbon atoms (i.e., C₂₋₁₀ alkynylene). In certain embodiments, an alkynylene comprises two to eight carbon atoms (i.e., C₂₋₈ alkynylene). In other embodiments, an alkynylene comprises two to five carbon atoms (i.e., C₂₋₅ alkynylene). In other embodiments, an alkynylene comprises two to four carbon atoms (i.e., C₂₋₄ alkynylene). In other embodiments, an alkynylene comprises two to three carbon atoms (i.e., C₂₋₃ alkynylene). In other embodiments, an alkynylene comprises two carbon atoms (i.e., C₂ alkynylene). In other embodiments, an alkynylene comprises five to eight carbon atoms (i.e., C₅₋₈ alkynylene). In other embodiments, an alkynylene comprises three to five carbon atoms (i.e., C₃₋₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to saturated hydrocarbon groups, including straight-chain alkyl and branched-chain alkyl groups that contain from 1 to 6 carbons. The term —C_{x-y} alkylene- refers to a alkylene chain with from x to y carbons in the alkylene chain. For example, —C₁₋₆ alkylene- may be selected from methylene, ethylene, propylene, butylene, pentylene, and hexylene, any one of which may be optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene- refers to a alkenylene chain with from x to y carbons in the alkenylene chain. For example, —C₂₋₆ alkenylene- may be selected from ethenylene, propenylene, butenylene, pentenylene, and hexenylene, any one of which may be optionally substituted. An alkenylene chain may have one double bond or more than one double bond in the alkenylene chain. The term —C_{x-y} alkynylene- refers to a alkynylene chain with from x to y carbons in the alkynylene chain. For example, —C₂₋₆ alkynylene- may be selected from ethynylene, propynylene, butynylene, pentynylene, and hexynylene, any one of which may be optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle includes 3- to 10-membered monocyclic rings and polycyclic rings (e.g., 6- to 12-membered bicyclic rings). Each ring of a polycyclic carbocycle may be selected from saturated, unsaturated, and aromatic rings. Polycyclic carbocycles may be fused, bridged or spiro-ring systems. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

The term “carbocyclene” as used herein refers to a divalent saturated, unsaturated or aromatic ring in which each atom of the ring is carbon. The carbocyclene is attached to the rest of the molecule through a single bond and to the radical group through a single bond. A carbocyclene may be optionally substituted by one or more substituents such as those substituents described herein. Carbocyclene includes divalent 3- to 10-membered monocyclic rings and divalent polycyclic rings (e.g., 6- to 12-membered bicyclic rings). Each ring of a polycyclic carbocyclene may be selected from saturated, unsaturated, and aromatic rings. Polycyclic carbocyclenes may be fused, bridged or spiro-ring systems. Polycyclic carbocyclenes may be fused, bridged or spiro-ring systems. The single bond connecting the carbocyclene to the rest of the molecule and the single bond connecting the carbocyclene to the radical group may be located on the same ring or different rings of a polycyclic carbocyclene. In some embodiments, the carbocycle is an arylene, for example, a phenylene. A “phenylene” as used herein refers to a divalent benzene group. The phenylene is attached to the rest of the molecule through a single bond and to the radical group through a single bond. A phenylene may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused, bridged, or spiro-ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic polycyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) p-electron system in accordance with the Hickel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any one of which may be optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and polycyclic rings (e.g., 6- to 12-membered bicyclic rings). Polycyclic heterocycles may be fused, bridged or spiro-ring systems. Each ring of a polycyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

The term “heterocyclene” as used herein refers to a divalent saturated, unsaturated, non-aromatic or aromatic ring comprising one or more heteroatoms. Exemplary heteroatoms include N, O, Si, P, B, and S atoms. The heterocyclene is attached to the rest of the molecule through a single bond and to the radical group through a single bond. The single bond attaching the heterocyclene group to the rest of the molecule and the single bond attaching the heterocyclene group to the radical group may be each independently connected through any atom of the heterocyclene as valency permits, including a carbon atom in the heterocyclene ring or a heteroatom in the heterocyclene ring. A heterocyclene may be optionally substituted by one or more substituents such as those substituents described herein. Heterocyclenes include 3- to 10-membered monocyclic rings and polycyclic rings (e.g., 6- to 12-membered bicyclic rings). Each ring of a polycyclic heterocyclene may be selected from saturated, unsaturated, and aromatic rings. Polycyclic heterocyclenes may be fused, bridged or spiro-ring systems. The single bond connecting the heterocyclene to the rest of the molecule and the single bond connecting the heterocyclene to the radical group may be located on the same ring or different rings of a polycyclic heterocyclene and may be attached to the rest of the molecule or the radical group through any atom of the heterocyclene, valence permitting, such as a carbon or nitrogen atom of the heterocycle. In some embodiments, the heterocyclene comprises at least one heteroatom selected from oxygen, nitrogen, sulfur, or any combination thereof. In some embodiments, the heterocyclene comprises at least one heteroatom selected from oxygen, nitrogen, or any combination thereof. In some embodiments, the heterocyclene comprises at least one heteroatom selected from oxygen, sulfur, or any combination thereof. In some embodiments, the heterocyclene comprises at least one heteroatom selected from nitrogen, sulfur, or any combination thereof. In some embodiments, the heterocyclene is a heteroarylene. In some embodiments, the heterocyclene is a heterocycloalkylene.

“Heterocycloalkyl” refers to a stable 3 to 12 membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused, bridged, or spiro-ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, and 1,1-dioxothiomorpholinyl. Heterocycloalkyl may be optionally substituted by one or more substituents such as those substituents described herein.

The term “heteroaryl” refers to a radical derived from a 5- to 12-membered aromatic ring radical whose ring structure comprise at least one heteroatom, preferably between one to four heteroatoms. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems wherein at least one of the rings in the ring system is aromatic, i.e., it contains a cyclic, delocalized (4n+2) p-electron system in accordance with the Hickel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocyclic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radical, each halogen may be independently selected for example, 1-chloro,2-fluoroethane.

The term “adjacent” refers to the connectivity of moieties where adjacent moieties are defined as being covalently attached or bonded to another atom. For example, -L¹-L²-L³-L⁴-, L¹ and L² are adjacent, L² and L³ are adjacent, and L³ and L⁴ are adjacent or

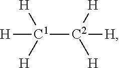

where C¹ and C² are adjacent. Adjacent substituents refers to substituents attached to different atoms wherein the two atoms are covalently attached, e.g. for CH₂R^{w1}CH₂R^{w2}, where the R^{w1} and R^{w2} are adjacent substituents.

The term “substituted” refers to moieties having substituents replacing a hydrogen on one or more carbons or substitutable heteroatoms, e.g., an NH or NH₂ of a compound. Unless specified otherwise (e.g., by using the terms “substituted” or “optionally substituted”, or by the inclusion of an “—R” group), chemical groups described herein are unsubstituted. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)R^{a}, —R^{b}—OC(O)OR^{a}, —R^{b}—OC(O)N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 0, 1, or 2), —R^{b}—S(O)ₜOR^{a} (where t is 1 or 2), —R^{b}—S(O)ₜN(R^{a})₂ (where t is 1 or 2), and —P(O)(R^{a})₂; and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any one of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)R^{a}, —R^{b}—OC(O)OR^{a}, —R^{b}—OC(O)N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 0, 1, or 2), —R^{b}—S(O)OR^{a} (where t is 1 or 2), —R^{b}—S(O)ₜN(R^{a})₂ (where t is 1 or 2), and —P(O)(R^{a})₂; wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a},
—R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b} —C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 0, 1, or 2), —R^{b}—S(O)OR^{a} (where t is 1 or 2), —R^{b}—S(O)ₜN(R^{a})₂ (where t is 1 or 2), and —P(O)(R^{a})₂; and wherein each R^{b} is independently selected from a direct bond or a straight or branched alkylene, alkenylene, or alkynylene chain, and each R¹⁰ is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers”, and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers”, and “administering” a compound should be understood to mean providing a compound or salt of the invention or a prodrug of a compound or salt of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In one aspect, the present disclosure provides a compound represented by the structure of Formula (A):

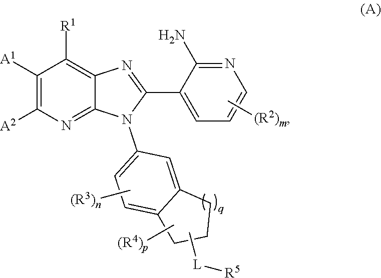

or a pharmaceutically acceptable salt thereof;
wherein:
R¹ is selected from:
   hydrogen, halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN;
   C₁₋₆ alkyl, C₂₋₆ alkenyl, and C₂₋₆ alkynyl, any of which is optionally substituted with one or more substituents independently selected from: halogen —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN; and
   3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, ═O, —NO₂, —CN, C₁₋₆ alkyl, and C₁₋₆ haloalkyl;
A¹ and A² are each independently selected from:
   hydrogen, halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
   C₁₋₆ alkyl, C₂₋₆ alkenyl, and C₂₋₆ alkynyl, any of which is optionally substituted with one or more substituents independently selected from: halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   5- to 10-membered heterocycle and C₃₋₁₀ carbocycle, any of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
      C₁₋₆ alkyl, C₂₋₆ alkenyl, and C₂₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
      C₃₋₁₀ carbocycle and 3- to 10-membered heterocycle; wherein the C₃₋₁₀ carbocycle and 3- to 10-membered heterocycle are each optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
         C₁₋₆ alkyl, C₂₋₆ alkenyl, and C₂₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN;
q is selected from 1, 2, and 3;
m and n are each independently selected from 0, 1, 2, and 3;
R² is independently selected at each instance from halogen, C₁₋₄ alkyl, C₁₋₄ haloalkyl, —OR¹², —SR¹², —N(R¹²)₂, —NO₂, and —CN;
R³ is independently selected at each instance from:
   halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
   C₁₋₆ alkyl optionally substituted with one or more substituents independently selected from: halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN;
p is selected from 0, 1, 2, 3, 4, and 5;
R⁴ is independently selected at each instance from:
   halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
   C₁₋₆ alkyl, C₂₋₆ alkenyl, C₂₋₆ alkynyl, any one of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
two R⁴ attached to the same atom are taken together to form a group selected from:
   ═O, ═S, and ═N(R¹⁴); or
two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from: halogen, C₁₋₄ alkyl, C₁₋₄ haloalkyl, —OR¹⁴, —SR¹⁴, —N(R¹⁴)₂, —NO₂, and —CN;
L is a bond or represented by -L¹-L²-L³-L⁴-, wherein L¹, L², L³, and L⁴ are each independently selected from (a) and (b):
   (a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
   (b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
      wherein L², L³, and L⁴ are each optionally absent;
      wherein no more than two of L¹, L², L³, and L⁴ are selected from (a) and the two selected are not adjacent;
R⁵ is selected from 3- to 12-membered heterocycle and C₃₋₁₂carbocycle, any of which is substituted by —C(O)H or —C(O)D, wherein R⁵ is further optionally substituted by one or more substituents independently selected from:
   halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN;
   C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
   4- to 6-membered heterocycle optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), —CN, C₁₋₆ alkyl, and C₁₋₆ haloalkyl;
R¹⁰, R¹¹, R¹², R¹³, R¹⁴, and R¹⁵ are each independently selected at each occurrence from:
   hydrogen, C₁₋₄ alkyl, C₃₋₈ carbocycle, 3- to 8-membered heterocycle, and C₁₋₄ haloalkyl;
R¹⁶ is independently selected at each occurrence from:
   hydrogen;
   C₁₋₄ alkyl, wherein the C₁₋₄ alkyl is optionally substituted with one or more substituents independently selected from:
      halogen, —OR²⁰, —SR²⁰, —N(R²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
      C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
   C₃₋₈ carbocycle and 3- to 8-membered heterocycle, either of which is optionally substituted with one or more substituents independently selected from:
      halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰,
         —S(O)₂R²⁰, —NO₂, and —CN; and
R²⁰ is independently selected at each occurrence from hydrogen, C₁₋₄ alkyl, C₁₋₄ haloalkyl, C₃₋₈ carbocycle, and 3- to 8-membered heterocycle.

In some embodiments, for the compound or salt of Formula (A), R¹ is selected from hydrogen, halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from hydrogen, halogen, —OR¹⁰, —SR¹⁰, —N(R¹⁰)₂, —C(O)N(R¹⁰)₂, —C(O)OR¹⁰, —OC(O)R¹⁰, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from hydrogen, halogen, —OR¹⁰, —SR¹⁰, —N(R¹⁰)₂, —C(O)N(R¹⁰)₂, —C(O)OR¹⁰, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from hydrogen, halogen, —OR¹⁰, —SR¹⁰, —N(R¹⁰)₂, —C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from hydrogen, halogen, —OR¹⁰, —N(R¹⁰)₂, —S(O)₂R¹⁰, —N(R¹⁰)S(O)₂R¹⁰, —S(O)₂N(R¹⁰)₂, and —CN. In some embodiments, R¹ is selected from hydrogen, fluoro, chloro, —OR¹⁰, —N(R¹⁰)₂, —N(R¹⁰)S(O)₂R¹⁰, and —S(O)₂N(R¹⁰)₂. In some embodiments, R¹ is selected from hydrogen, fluoro, chloro, —N(R¹⁰)₂, —N(R¹⁰)S(O)₂R¹⁰, and —S(O)₂N(R¹⁰)₂. In some embodiments, R¹ is selected from hydrogen, fluoro, chloro, —N(R¹⁰)₂, and —N(R¹⁰)S(O)₂R¹⁰. In some embodiments, R¹ is selected from hydrogen, —N(R¹⁰)₂, and —N(R¹⁰)S(O)₂R¹⁰. In some embodiments, R¹ is selected from —N(R¹⁰)S(O)₂R¹⁰. In some embodiments, R¹ is selected from

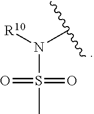

In some embodiments, R¹ is

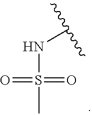 [SMILES: *NS(C)(=O)=O]

In some embodiments, for the compound or salt of Formula (A), R¹ is selected from C₁₋₆ alkyl, C₂₋₆ alkenyl, and C₂₋₆ alkynyl, any of which is optionally substituted with one or more substituents independently selected from: halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from C₁₋₆ alkyl and C₂₋₆ alkynyl, any of which is optionally substituted with one or more substituents independently selected from: halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from C₁₋₆ alkyl and C₂₋₆ alkynyl, any of which is optionally substituted with one or more substituents independently selected from: halogen, —OR¹⁰, —SR¹⁰, —N(R¹⁰)₂, —C(O)N(R¹⁰)₂, —C(O)OR¹⁰, —OC(O)R¹⁰, —S(O)R¹⁰, —S(O)₂R¹⁰, —N(R¹⁰)S(O)₂R¹⁰, —S(O)₂N(R¹⁰)₂, —NO₂, and —CN. In some embodiments, R¹ is selected from C₁₋₆ alkyl and C₂₋₆ alkynyl, any of which is optionally substituted with one or more substituents independently selected from: halogen, —OR¹⁰, —SR¹⁰, —N(R¹⁰)₂, —C(O)N(R¹⁰)₂, —C(O)OR¹⁰, —S(O)₂R¹⁰, —N(R¹⁰)S(O)₂R¹⁰, —S(O)₂N(R¹⁰)₂, —NO₂, and —CN. In some embodiments, R¹ is selected from C₁₋₆ alkyl and C₂₋₆ alkynyl, any of which is optionally substituted with one or more substituents independently selected from: halogen, —OR¹⁰, —SR¹⁰, —N(R¹⁰)₂, —C(O)N(R¹⁰)₂, —C(O)OR¹⁰, —S(O)₂R¹⁰, —N(R¹⁰)S(O)₂R¹⁰, —S(O)₂N(R¹⁰)₂, —NO₂, and —CN. In some embodiments, R¹ is selected from C₁₋₄ alkyl and C₂₋₄ alkynyl, any of which is optionally substituted with one or more substituents independently selected from: halogen, —OR¹⁰, —SR¹⁰, —N(R¹⁰)₂, —C(O)N(R¹⁰)₂, —C(O)OR¹⁰, —S(O)₂R¹⁰, —N(R¹⁰)S(O)₂R¹⁰, —S(O)₂N(R¹⁰)₂, —NO₂, and —CN. In some embodiments, R¹ is selected from C₁₋₆ alkyl and C₂₋₆ alkynyl, any of which is optionally substituted with one or more substituents independently selected from: halogen, —OR¹⁰, —SR¹⁰, —N(R¹⁰)₂, —N(R¹⁰)S(O)₂R¹⁰, —NO₂, and —CN. In some embodiments, R¹ is selected from C₁₋₆ alkyl and C₂₋₆ alkynyl, each of which is optionally substituted with one or more substituents independently selected from any of which is optionally substituted with one or more substituents independently selected from: halogen, —OR¹⁰, —SR¹⁰, —N(R¹⁰)₂, —NO₂, and —CN. In some embodiments, R¹ is selected from C₁₋₆ alkyl and C₂₋₆ alkynyl, each of which is optionally substituted with one or more substituents independently selected from any of which is optionally substituted with one or more substituents independently selected from: halogen, —OR¹⁰, —SR¹⁰, and —N(R¹⁰)₂. In some embodiments, R¹ is selected from C₁₋₆ alkyl and C₂₋₆ alkynyl, each of which is optionally substituted with one or more substituents independently selected from any of which is optionally substituted with one or more substituents independently selected from: fluoro, chloro, —OR¹⁰, and —N(R¹⁰)₂. In some embodiments, R¹ is selected from C₁₋₆ alkyl and C₂₋₆ alkynyl, each of which is optionally substituted with one or more substituents independently selected from any of which is optionally substituted with one or more substituents independently selected from: fluoro, —OR¹⁰, and —N(R¹⁰)₂. In some embodiments, R¹ is selected from C₁₋₆ alkyl and C₂₋₆ alkynyl, each of which is optionally substituted with one or more substituents independently selected from any of which is optionally substituted with one or more substituents independently selected from: —OR¹⁰ and —N(R¹⁰)₂. In some embodiments, R¹ is selected from C₁₋₆ alkyl and C₂₋₆ alkynyl, each of which is optionally substituted with one or more substituents independently selected from any of which is optionally substituted with one or more substituents independently selected from: —OR¹⁰. In some embodiments, R¹ is selected from C₁₋₄ alkyl and C₂₋₄ alkynyl, each of which is optionally substituted with one or more substituents independently selected from any of which is optionally substituted with one or more substituents independently selected from: —OR¹⁰. In some embodiments, R¹ is selected from C₁₋₆ alkyl and C₂₋₆ alkynyl, each of which is optionally substituted with one or more substituents independently selected from any of which is optionally substituted with one or more substituents independently selected from: —OCH₃, —OCH₂CH₃, —OCH(CH₃)₂, —OCH₂CH₂CH₃, —OCH₂CH₂CH₂CH₃, —OCH(CH₃)CH₂CH₃, —OCH₂CH(CH₃)CH₃, —OC(CH₃)₃, and —OPh. In some embodiments, R¹ is selected from C₁₋₆ alkyl and C₂₋₆ alkynyl, each of which is optionally substituted with one or more substituents independently selected from any of which is optionally substituted with one or more substituents independently selected from: —OCH₃, —OCH₂CH₃, —OCH(CH₃)₂, and —OC(CH₃)₃. In some embodiments, R¹ is selected from C₁₋₆ alkyl and C₂₋₆ alkynyl, each of which is optionally substituted with one or more substituents independently selected from any of which is optionally substituted with one or more substituents independently selected from: —OCH₃. In some embodiments, R¹ is selected from

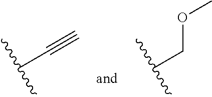

Cl. In some embodiments, R¹ is

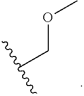 [SMILES: *COC]

In some embodiments, R¹ is selected from C₁₋₆ alkyl and C₂₋₆ alkynyl. In some embodiments, R¹ is selected from C₁₋₆ alkyl. In some embodiments, R¹ is selected from C₂₋₆ alkynyl. In some embodiments, R¹ is

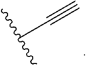 [SMILES: *C#C]

In some embodiments, for the compound or salt of Formula (A), R¹ is selected from C₁₋₆ alkyl, C₂₋₆ alkenyl, and C₂₋₆ alkynyl. In some embodiments, R¹ is selected from C₁₋₆ alkyl and C₂₋₆ alkynyl. In some embodiments, R¹ is selected from C₁₋₆ alkyl and C₂₋₆ alkynyl. In some embodiments, R¹ is selected from C₁₋₆ alkyl and C₂₋₆ alkynyl. In some embodiments, R¹ is selected from C₁₋₆ alkyl. In some embodiments, R¹ is selected from C₂₋₆ alkynyl.

In some embodiments, for the compound or salt of Formula (A), R¹ is selected from 3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, ═O, —NO₂, —CN, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)N(R¹⁰)₂, —C(O)OR¹⁰, —OC(O)R¹⁰, —S(O)₂R¹⁰, —N(R¹⁰)S(O)₂R¹⁰, —S(O)₂N(R¹⁰)₂, ═O, —NO₂, —CN, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)OR¹⁰, —N(R¹⁰)S(O)₂R¹⁰, —S(O)₂N(R¹⁰)₂, ═O, —NO₂, —CN, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)OR¹⁰, ═O, —NO₂, —CN, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: halogen, —OR¹⁰, —SR¹⁰, —N(R¹⁰)₂, ═O, —NO₂, —CN, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: halogen, —OR¹⁰, —N(R¹⁰)₂, ═O, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: fluoro, chloro, —OR¹⁰, ═O, C₁₋₆ alkyl, and C₁₋₆ haloalkyl. In some embodiments, R¹ is selected from 3- to 6-membered heterocycle and C₃₋₆ carbocycle, any of which is optionally substituted with one or more substituents independently selected from: fluoro, chloro, —OCH₃, —OCH₂CH₃, —OCH(CH₃)₂, —OC(CH₃)₃, ═O, C₁₋₆ alkyl, —CH₂F, —CHF₂, —CF₃, and —CF₂CF₃. In some embodiments, R¹ is selected from 4- to 6-membered heterocycle and C₃₋₅ carbocycle, any of which is optionally substituted with one or more substituents independently selected from: halogen, —OR¹⁰, —SR¹⁰, —N(R¹⁰)₂, ═O, —NO₂, —CN, C₁₋₆ alkyl, and C₁₋₆ haloalkyl. In some embodiments, R¹ is selected from 4- to 6-membered heterocycle, cyclopropyl, and cyclobutyl, any of which is optionally substituted with one or more substituents independently selected from: halogen, —OR¹⁰, —SR¹⁰, —N(R¹⁰)₂, ═O, —NO₂, —CN, C₁₋₆ alkyl, and C₁₋₆ haloalkyl.

In some embodiments, for the compound or salt of Formula (A), R¹ is selected from azetidinyl, imidazolyl, triazolyl, morpholinyl, piperazinyl, and cyclopropyl, any of which is optionally substituted with one or more substituents independently selected from: halogen, —OR¹⁰, —SR¹⁰, —N(R¹⁰)₂, ═O, —NO₂, —CN, C₁₋₆ alkyl, and C₁₋₆ haloalkyl. In some embodiments, R¹ is selected from azetidinyl, imidazolyl, triazolyl, morpholinyl, piperazinyl, and cyclopropyl, any of which is optionally substituted with one or more substituents independently selected from: fluoro, chloro, —OR¹⁰, —N(R¹⁰)₂, ═O, C₁₋₆ alkyl, and C₁₋₆ haloalkyl. In some embodiments, R¹ is selected from azetidinyl, imidazolyl, triazolyl, morpholinyl, piperazinyl, and cyclopropyl, any of which is optionally substituted with one or more substituents independently selected from: fluoro, chloro, OCH₃, —OCH₂CH₃, —OCH(CH₃)₂, —OC(CH₃)₃, ═O, C₁₋₆ alkyl, and C₁₋₆ haloalkyl. In some embodiments, R¹ is selected from azetidinyl, imidazolyl, triazolyl, morpholinyl, piperazinyl, and cyclopropyl, any of which is optionally substituted with one or more substituents independently selected from: fluoro, chloro, —OCH₃, —OCH₂CH₃, ═O, —CH₃, —CH₂CH₃, —CH(CH₃)₂, —CH₂F, —CHF₂, —CF₃, and —CF₂CF₃. In some embodiments, R¹ is selected from azetidinyl, imidazolyl, triazolyl, morpholinyl, piperazinyl, and cyclopropyl, any of which is optionally substituted with one or more substituents independently selected from: fluoro, ═O, —CH₃, —CH₂CH₃, —CH(CH₃)₂, —CH₂F, —CHF₂, and —CF₃. In some embodiments, R¹ is selected from azetidinyl, imidazolyl, triazolyl, morpholinyl, piperazinyl, and cyclopropyl, any of which is optionally substituted with one or more substituents independently selected from: ═O and —CH₃.

In some embodiments, for the compound or salt of Formula (A), R¹ is selected from

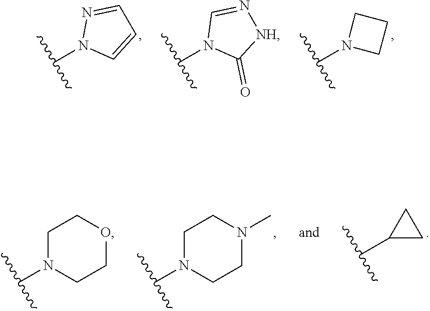

In some embodiments, R¹ is selected from

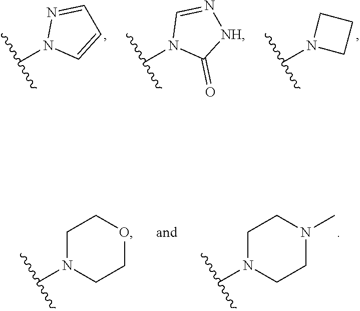

In some embodiments, R¹ is selected from

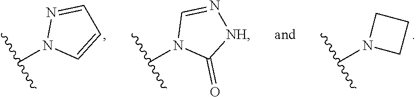 [SMILES: *n1cccn1 ; *n1cn[nH]c1=O ; *N1CCC1]

In some embodiments, R¹ is selected from

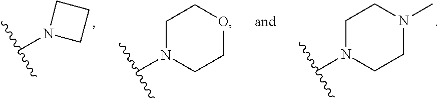

In some embodiments, R¹ is selected from

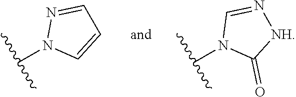 [SMILES: *n1cccn1 ; *n1cn[nH]c1=O]

In some embodiments, R¹ is selected from and

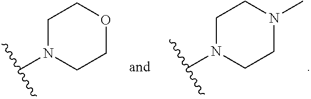

In some embodiments, R¹ is selected from

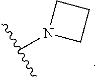 [SMILES: *N1CCC1]

In some embodiments, for the compound or salt of Formula (A), R⁵ is 3- to 12-membered heterocycle substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN;
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
4- to 6-membered heterocycle optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), —CN, C₁₋₆ alkyl, and C₁₋₆ haloalkyl.

In some embodiments, for the compound or salt of Formula (A), R⁵ is 3- to 12-membered heterocycle substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN.

In some embodiments, for the compound or salt of Formula (A), R⁵ is selected from 3- to 8-membered monocyclic heterocycle and 6- to 12-membered bicyclic heterocycle, each of which is substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN;
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
4- to 6-membered heterocycle optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), —CN, C₁₋₆ alkyl, and C₁₋₆ haloalkyl.

In some embodiments, for the compound or salt of Formula (A), R⁵ is selected from 3- to 8-membered monocyclic heterocycle and 6- to 12-membered bicyclic heterocycle, each of which is substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN.

In some embodiments, for the compound or salt of Formula (A), R⁵ is selected from 3- to 8-membered monocyclic heterocycle and 6- to 12-membered bicyclic heterocycle, each of which is substituted by —C(O)H or —C(O)D, wherein R⁵ is further optionally substituted by one or more substituents. In some embodiments, R⁵ is selected from 3- to 8-membered monocyclic heterocycle, which is substituted by —C(O)H or —C(O)D, wherein R⁵ is further optionally substituted by one or more substituents. In some embodiments, R⁵ is selected from 6- to 12-membered bicyclic heterocycle, which is substituted by —C(O)H or —C(O)D, wherein R⁵ is further optionally substituted by one or more substituents.

In some embodiments, for the compound or salt of Formula (A), R⁵ is 6- to 12-membered bicyclic heterocycle, each of which is substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN.

In some embodiments, for the compound or salt of Formula (A), R⁵ is 6- to 12-membered bicyclic heterocycle, each of which is substituted by —C(O)H or —C(O)D, wherein R⁵ is further optionally substituted by one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and C₁₋₆ alkyl which is optionally substituted with one or more substituents independently selected from: halogen, —OR¹⁶, —SR¹⁶, —N(R¹⁶)₂, —C(O)R¹⁶, —C(O)N(R¹⁶)₂, —C(O)OR¹⁶, —NO₂, ═O, ═S, ═N(R¹⁶), and —CN. In some embodiments, R⁵ is 6- to 12-membered bicyclic heterocycle, each of which is substituted by —C(O)H or —C(O)D, wherein R⁵ is further optionally substituted by one or more substituents independently selected from: halogen, —OR¹⁶, —N(R¹⁶)₂, —C(O)R¹⁶, —C(O)N(R¹⁶)₂, —C(O)OR¹⁶, —N(R¹⁶)S(O)₂R¹⁶, —S(O)₂N(R¹⁶)₂, —NO₂, ═O, ═S, ═N(R¹⁶), —N₃, and —CN; and C₁₋₆ alkyl which is optionally substituted with one or more substituents independently selected from: halogen, —OR¹⁶, —SR¹⁶, —N(R¹⁶)₂, —C(O)R¹⁶, —C(O)N(R¹⁶)₂, —C(O)OR¹⁶, —NO₂, ═O, ═S, ═N(R¹⁶), and —CN.

In some embodiments, for the compound or salt of Formula (A), R⁵ is 6- to 12-membered bicyclic heterocycle, each of which is substituted by —C(O)H or —C(O)D, wherein R⁵ is further optionally substituted by one or more substituents independently selected from halogen, —OR¹⁶, —N(R¹⁶)₂, —C(O)R¹⁶, —C(O)N(R¹⁶)₂, —C(O)OR¹⁶, —N(R¹⁶)S(O)₂R¹⁶, —S(O)₂N(R¹⁶)₂, —NO₂, ═O, ═S, ═N(R¹⁶), —N₃, and —CN; and C₁₋₆ alkyl optionally substituted by one or more substituents independently selected from halogen, —OR¹⁶, —N(R¹⁶)₂, —C(O)R¹⁶, —C(O)N(R¹⁶)₂, —C(O)OR¹⁶, —NO₂, ═O, ═S, ═N(R¹⁶), and —CN. In some embodiments, R⁵ is 6- to 12-membered bicyclic heterocycle, each of which is substituted by —C(O)H or —C(O)D, wherein R⁵ is further optionally substituted by one or more substituents independently selected from halogen, —OR¹⁶, —N(R¹⁶)₂, ═O, ═S, ═N(R¹⁶), and —CN; and C₁₋₆ alkyl optionally substituted by one or more substituents independently selected from halogen, —OR¹⁶, —N(R¹⁶)₂, and —CN. In some embodiments, R⁵ is 6- to 12-membered bicyclic heterocycle, each of which is substituted by —C(O)H or —C(O)D, wherein R⁵ is further optionally substituted by one or more substituents independently selected from halogen, —OR¹⁶, —N(R¹⁶)₂, and ═O; and C₁₋₆ alkyl optionally substituted by one or more substituents independently selected from halogen and —OR¹⁶. In some embodiments, R⁵ is 6- to 12-membered bicyclic heterocycle, each of which is substituted by —C(O)H or —C(O)D, wherein R⁵ is further optionally substituted by one or more substituents independently selected from fluoro, chloro, and ═O; and C₁₋₆ alkyl optionally substituted by one or more substituents independently selected from fluoro. In some embodiments, R⁵ is 6- to 12-membered bicyclic heterocycle, each of which is substituted by —C(O)H or —C(O)D, wherein R⁵ is further optionally substituted by one or more substituents independently selected from ═O; and C₁₋₆ alkyl. In some embodiments, R⁵ is 6- to 12-membered bicyclic heterocycle, each of which is substituted by —C(O)H or —C(O)D, wherein R⁵ is further optionally substituted by one or more substituents independently selected from ═O.

In some embodiments, for the compound or salt of Formula (A), R⁵ is

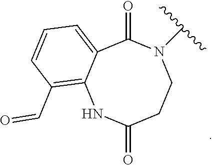 [SMILES: *N1CCC(=O)Nc2c(C=O)cccc2C1=O]

In some embodiments, for the compound or salt of Formula (A), L is a bond; and R⁵ is selected from

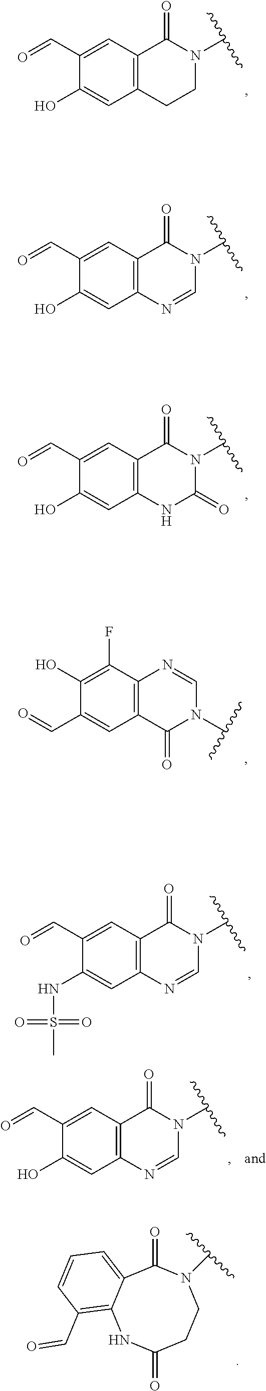

In some embodiments, L is a bond; and R⁵ is selected from

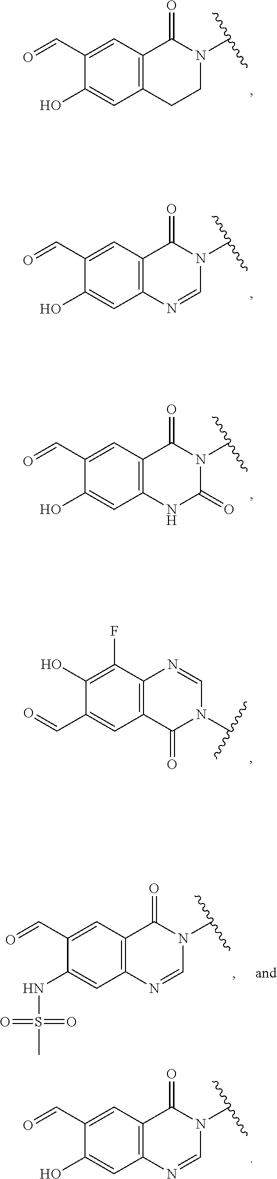 [SMILES: *N1CCc2cc(O)c(C=O)cc2C1=O ; *n1cnc2cc(O)c(C=O)cc2c1=O ; *n1c(=O)[nH]c2cc(O)c(C=O)cc2c1=O ; *n1cnc2c(F)c(O)c(C=O)cc2c1=O ; *n1cnc2cc(NS(C)(=O)=O)c(C=O)cc2c1=O ; *n1cnc2cc(O)c(C=O)cc2c1=O]

In some embodiments, L is a bond; and R⁵ is selected from

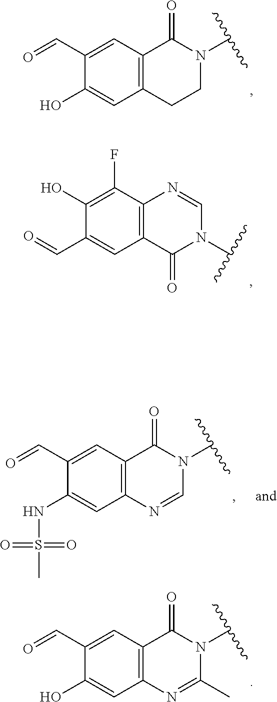 [SMILES: *N1CCc2cc(O)c(C=O)cc2C1=O ; *n1cnc2c(F)c(O)c(C=O)cc2c1=O ; *n1cnc2cc(NS(C)(=O)=O)c(C=O)cc2c1=O ; *n1c(C)nc2cc(O)c(C=O)cc2c1=O]

In some embodiments, L is a bond; and R⁵ is selected from

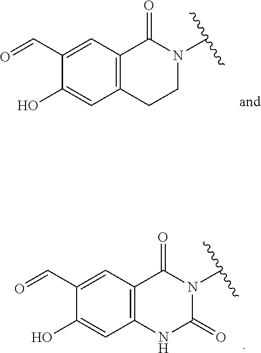 [SMILES: *N1CCc2cc(O)c(C=O)cc2C1=O ; *n1c(=O)[nH]c2cc(O)c(C=O)cc2c1=O]

In some embodiments, L is a bond; and R⁵ is selected from

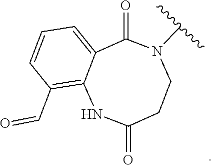 [SMILES: *N1CCC(=O)Nc2c(C=O)cccc2C1=O]

In some embodiments, the compound or salt of Formula (A), R⁵ is selected from 3- to 12-membered heterocycle and C₃₋₁₂carbocycle, any of which is substituted by —C(O)H or —C(O)D, wherein R⁵ is further optionally substituted by one or more substituents. In some embodiments, R⁵ is selected from 3- to 8-membered monocyclic heterocycle, 6- to 12-membered bicyclic heterocycle, and C₃₋₆ carbocycle, each of which is substituted by —C(O)H or —C(O)D, wherein R⁵ is further optionally substituted by one or more substituents. In some embodiments, R⁵ is selected from 3- to 8-membered monocyclic heterocycle and 6- to 12-membered bicyclic heterocycle, each of which is substituted by —C(O)H or —C(O)D, wherein R⁵ is further optionally substituted by one or more substituents. In some embodiments, R⁵ is selected from 3- to 8-membered monocyclic heterocycle, which is substituted by —C(O)H or —C(O)D, wherein R⁵ is further optionally substituted by one or more substituents. In some embodiments, R⁵ is selected from 6- to 12-membered bicyclic heterocycle, which is substituted by —C(O)H or —C(O)D, wherein R⁵ is further optionally substituted by one or more substituents. In some embodiments, R⁵ is selected from C₃₋₆ carbocycle, which is substituted by —C(O)H or —C(O)D, wherein R⁵ is further optionally substituted by one or more substituents. In some embodiments, R⁵ is selected from phenyl, which is substituted by —C(O)H or —C(O)D, wherein R⁵ is further optionally substituted by one or more substituents.

In some embodiments, for the compound or salt of Formula (A), R⁵ is C₃₋₆ carbocycle substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN;
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
4- to 6-membered heterocycle optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), —CN, C₁₋₆ alkyl, and C₁₋₆ haloalkyl.

In some embodiments, for the compound or salt of Formula (A), R⁵ is C₃₋₆ carbocycle substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN.

In some embodiments, for the compound or salt of Formula (A), R⁵ is C₃₋₆ carbocycle substituted by —C(O)H or —C(O)D, wherein R⁵ is further optionally substituted by one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and C₁₋₆ alkyl which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN. In some embodiments, R⁵ is phenyl substituted by —C(O)H or —C(O)D, wherein R⁵ is further optionally substituted by one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN; and C₁₋₆ alkyl which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN. In some embodiments, R⁵ is phenyl substituted by —C(O)H or —C(O)D, wherein R⁵ is further optionally substituted by one or more substituents independently selected from: halogen, —OR¹⁶, —SR¹⁶, —N(R¹⁶)₂—C(O)R¹⁶, —C(O)N(R¹⁶)₂, —C(O)OR¹⁶, —N(R¹⁶)S(O)₂R¹⁶, —S(O)₂N(R¹⁶)₂, —S(O)R¹⁶, —S(O)₂R¹⁶, —NO₂, ═O, ═S, ═N(R¹⁶), —N₃, and —CN; and C₁₋₆ alkyl which is optionally substituted with one or more substituents independently selected from: halogen, —OR¹⁶, —SR¹⁶, —N(R¹⁶)₂, —C(O)R¹⁶, —C(O)N(R¹⁶)₂, —C(O)OR¹⁶, —S(O)₂N(R¹⁶)₂, —S(O)R¹⁶, —S(O)₂R¹⁶, —NO₂, ═O, ═S, ═N(R¹⁶), and —CN. In some embodiments, R⁵ is phenyl substituted by —C(O)H or —C(O)D, wherein R⁵ is further optionally substituted by one or more substituents independently selected from: halogen, —OR¹⁶, —SR¹⁶, —N(R¹⁶)₂, —C(O)R¹⁶, —C(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, —CN; and C₁₋₆ alkyl, optionally substituted with one or more substituents independently selected from halogen, —OR¹⁶, —SR¹⁶, —N(R¹⁶)₂, —C(O)R¹⁶, —NO₂, ═O, ═S, ═N(R¹⁶), and —CNCl. In some embodiments, R⁵ is selected from

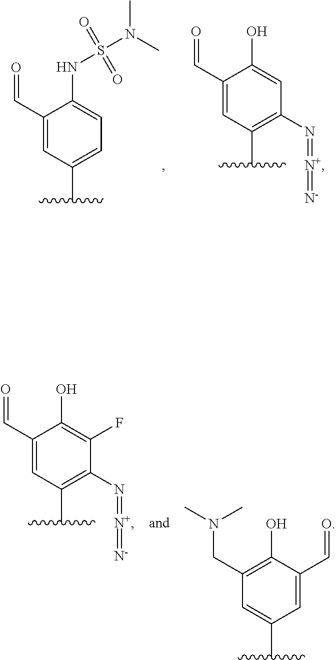

In some embodiments, R⁵ is selected from

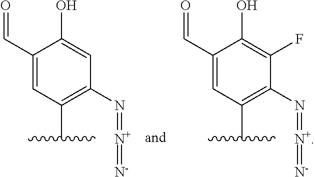

In some embodiments, R⁵ is selected from

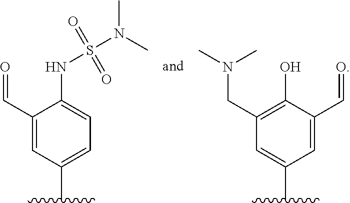 [SMILES: *c1ccc(NS(=O)(=O)N(C)C)c(C=O)c1 ; *c1cc(C=O)c(O)c(CN(C)C)c1]

In another aspect, Formula (A) is represented by the structure of Formula (A-1):

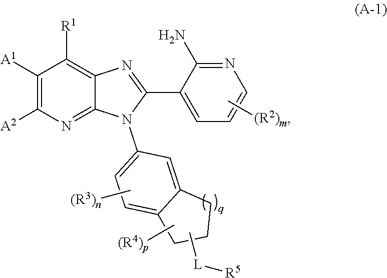

or a pharmaceutically acceptable salt thereof;
   wherein:
      R¹ is selected from hydrogen, halogen, C₁₋₄ alkyl, C₁₋₄ haloalkyl, —OR¹⁰, —SR¹⁰, —N(R¹⁰)₂, —NO₂, and —CN;
      A¹ and A² are each independently selected from:
         hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
         C₁₋₆ alkyl, C₂₋₆ alkenyl, and C₂₋₆ alkynyl, any of which is optionally substituted with one or more substituents independently selected from:
         halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
         5- to 10-membered heterocycle and C₃₋₁₀ carbocycle, any of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O) N(R¹¹)₂, —N(R¹¹)C(O)N(R¹¹)₂, —S(O)R¹¹, —S(O)₂R¹¹, —S(O)₂N(R¹¹)₂, —N O₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
         C₃₋₁₀ carbocycle and 3- to 10-membered heterocycle; wherein the C₃₋₁₀ carbocycle and 3- to 10-membered heterocycle are each optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN;
      q is selected from 1, 2, and 3;
      m and n are each independently selected from 0, 1, 2, and 3;
      R² is independently selected at each instance from halogen, C₁₋₄ alkyl, C₁₋₄ haloalkyl, —OR¹², —SR¹², —N(R¹²)₂, —NO₂, and —CN;
      R³ is independently selected at each instance from:
         halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
         C₁₋₆ alkyl optionally substituted with one or more substituents independently selected from:
         halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN;
      p is selected from 0, 1, 2, 3, 4, and 5;
      R⁴ is independently selected at each instance from:
         halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
         C₁₋₆ alkyl, C₂₋₆ alkenyl, C₂₋₆ alkynyl, any one of which is optionally substituted with one or more substituents independently selected from:
         halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
         two R⁴ attached to the same atom are taken together to form a group selected from:
         ═O, ═S, and ═N(R¹⁴); or
         two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from:
         halogen, C₁₋₄ alkyl, C₁₋₄ haloalkyl, —OR¹⁴, —SR¹⁴, —N(R¹⁴)₂, —NO₂, and —CN;
      L is represented by -L¹-L²-L³-L⁴-, wherein L¹, L², L³, and L⁴ are each independently selected from (a) and (b):
         (a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
         (b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
         wherein L², L³, and L⁴ are each optionally absent;
         wherein no more than two of L¹, L², L³, and L⁴ are selected from (a) and the two selected are not adjacent;
      R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from:
         halogen, C₁₋₄ alkyl, C₂₋₆ alkynyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
         4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN;
      R¹⁰, R¹¹, R¹², R¹³, R¹⁴, and R¹⁵ are each independently selected at each occurrence from: hydrogen, C₁₋₄ alkyl, C₃₋₈ carbocycle, 3- to 8-membered heterocycle, and C₁₋₄ haloalkyl;
      R¹⁶ is independently selected at each occurrence from:
         hydrogen;
         C₁₋₄ alkyl, wherein the C₁₋₄ alkyl is optionally substituted with one or more substituents independently selected from:
         halogen, —OR²⁰, —SR²⁰, —N(R²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN;
         C₃₋₈ carbocycle and 3- to 8-membered heterocycle, either of which is optionally substituted with one or more substituents independently selected from: halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
         C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —NO₂, and —CN; and
R²⁰ is independently selected at each occurrence from hydrogen, C₁₋₄ alkyl, C₁₋₄ haloalkyl, C₃₋₈ carbocycle, and 3- to 8-membered heterocycle.

In some embodiments, for the compound or salt of Formula (A) or (A-1), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from: halogen, C₁₋₄ alkyl, C₂₋₆ alkynyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and 4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN. In some embodiments, R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from: halogen, C₁₋₄ alkyl, C₂₋₆ alkynyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and 4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN; and R¹⁶ is selected from hydrogen, C₁₋₄ alkyl, C₁₋₄ haloalkyl, C₃₋₈ carbocycle and 3- to 8-membered heterocycle.

In some embodiments, for the compound or salt of Formula (A) or (A-1), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from: halogen, C₁₋₄ alkyl, C₂₋₆ alkynyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN. In some embodiments, R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from: halogen, C₁₋₄ alkyl, C₂₋₆ alkynyl, C₁₋₄ haloalkyl, —OR¹⁶, —SR¹⁶, —N(R¹⁶)₂, —C(O)R¹⁶, —C(O)N(R¹⁶)₂, —NO₂, ═O, ═S, ═N(R¹⁶), and —CN. In some embodiments, R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from: halogen, C₁₋₄ alkyl, C₂₋₆ alkynyl, C₁₋₄ haloalkyl, —OR¹⁶, and —CN.

In some embodiments, for the compound or salt of Formula (A) or (A-1), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from 4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN. In some embodiments, R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from 4- to 6-membered saturated heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN; and R¹⁶ is selected from hydrogen, C₁₋₄ alkyl, C₁₋₄ haloalkyl, C₃₋₈ carbocycle and 3- to 8-membered heterocycle.

In some embodiments, for the compound or salt of Formula (A) or (A-1), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶. In some embodiments, R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶; and R¹⁶ is selected from hydrogen, C₁₋₄ alkyl, C₁₋₄ haloalkyl, C₃₋₈ carbocycle and 3- to 8-membered heterocycle.

In some embodiments, for the compound or salt of Formula (A) or (A-1), R⁵ is C₃₋₁₀ carbocycle substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶. In some embodiments, R⁵ is C₃₋₁₀ carbocycle substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶; and —OR¹⁶; and R¹⁶ is selected from hydrogen, C₁₋₄ alkyl, C₁₋₄ haloalkyl, C₃₋₈ carbocycle and 3- to 8-membered heterocycle. In some embodiments, R⁵ is C₃₋₆ carbocycle substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶; and —OR¹⁶; and R¹⁶ is selected from hydrogen, C₁₋₄ alkyl, C₁₋₄ haloalkyl, C₃₋₈ carbocycle and 3- to 8-membered heterocycle. In some embodiments, R⁵ is phenyl substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶.

In some embodiments, for the compound or salt of Formula (A) or (A-1), R⁵ is phenyl substituted by —C(O)H or —C(O)D, wherein R⁵ is further optionally substituted by one or more substituents independently selected from halogen, C₁₋₄ alkyl, C₂₋₆ alkynyl, C₁₋₄ haloalkyl, and —OR¹⁶; and R¹⁶ is selected from hydrogen, C₁₋₄ alkyl, and C₁₋₄ haloalkyl. In some embodiments, R⁵ is phenyl substituted by —C(O)H, wherein R⁵ is further optionally substituted by one or more substituents independently selected from C₂₋₆ alkynyl and —OR¹⁶; and R¹⁶ is selected from hydrogen, C₁₋₄ alkyl, and C₁₋₄ haloalkyl. In some embodiments, R⁵ is selected from

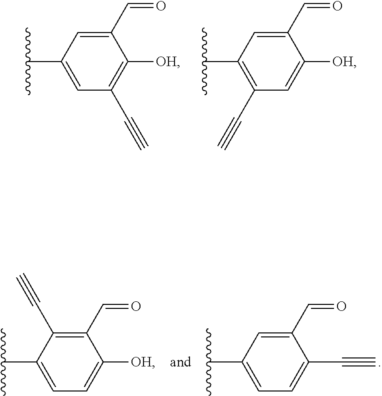

In some embodiments, R⁵ is

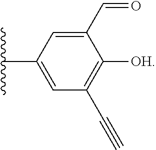 [SMILES: *c1cc(C#C)c(O)c(C=O)c1]

In some embodiments, R⁵ is

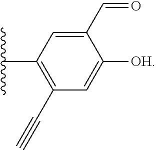 [SMILES: *c1cc(C=O)c(O)cc1C#C]

In some embodiments, R⁵ is

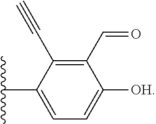 [SMILES: *c1ccc(O)c(C=O)c1C#C]

In some embodiments, R⁵ is

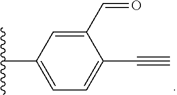 [SMILES: *c1ccc(C#C)c(C=O)c1]

In another aspect, Formula (A) or (A-1) is represented by the structure of Formula (I):

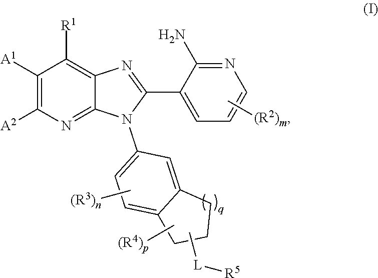

or a pharmaceutically acceptable salt thereof, wherein:
   R¹ is selected from hydrogen, halogen, C₁₋₄ alkyl, C₁₋₄ haloalkyl, —OR¹⁰, —SR¹⁰, —N(R¹⁰)₂, —NO₂, and —CN;
   A¹ and A² are each independently selected from:
      hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO 2, and —CN;
      C₁₋₆ alkyl, C₂₋₆ alkenyl, and C₂₋₆ alkynyl, any of which is optionally substituted with one or more substituents independently selected from:
         halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
      5- to 10-membered heterocycle and C₃₋₁₀ carbocycle, any of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹) 2, —NO₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
         C₃₋₁₀ carbocycle and 3- to 10-membered heterocycle; wherein the C₃₋₁₀ carbocycle and 3- to 10-membered heterocycle are each optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN; and
   q is selected from 1, 2, and 3;
   m and n are each independently selected from 0, 1, 2, and 3;
   R² is independently selected at each instance from halogen, C₁₋₄ alkyl, C₁₋₄ haloalkyl, —OR¹², —SR¹², —N(R¹²)₂, —NO₂, and —CN;
   R³ is independently selected at each instance from:
      halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
      C₁₋₆ alkyl optionally substituted with one or more substituents independently selected from
         halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN;
   p is selected from 0, 1, 2, 3, 4, and 5;
   R⁴ is independently selected at each instance from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
      C₁₋₆ alkyl, C₂₋₆ alkenyl, C₂₋₆ alkynyl, any one of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
      two R⁴ attached to the same atom are taken together to form a group selected from:
         ═O, ═S, and ═N(R¹⁴); or
      two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from: halogen, C₁₋₄ alkyl, C₁₋₄ haloalkyl, —OR¹⁴, —SR¹⁴, —N(R¹⁴)₂, —NO₂, and —CN;
   L is represented by -L¹-L²-L³-L⁴-, wherein L¹, L², L³, and L⁴ are each independently selected from (a) and (b):
      (a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
      (b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
      wherein L², L³, and L⁴ are each optionally absent;
      wherein no more than two of L¹, L², L³, and L⁴ are selected from (a) and the two selected are not adjacent;
   R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from:
      halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
      4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN;
   R¹⁰, R¹¹, R¹², R¹³, R¹⁴, and R¹⁵ are each independently selected at each occurrence from:
      hydrogen, C₁₋₄ alkyl, C₃₋₈ carbocycle, 3- to 8-membered heterocycle, and C₁₋₄ haloalkyl;
      R¹⁶ is independently selected at each occurrence from:
         hydrogen;
         C₁₋₄ alkyl, wherein the C₁₋₄ alkyl is optionally substituted with one or more substituents independently selected from:
         halogen, —OR²⁰, —SR²⁰, —N(R²⁰)₂, —C(O)R²⁰, —C(O)OR²⁰, —OC(O)R²⁰, —C(O)N(R²⁰) 2, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN;
      C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
      C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —NO₂, and —CN; and
   R²⁰ is independently selected at each occurrence from hydrogen, C₁₋₄ alkyl, C₁₋₄ haloalkyl, C₃₋₈ carbocycle, and 3- to 8-membered heterocycle.

In some embodiments, for the compound or salt of Formula (A), (A-1), or (I), q is selected from 1, 2, and 3. In some embodiments, q is selected from 1 and 2. In some embodiments, q is selected from 1 and 3. In some embodiments, q is selected from 2 and 3. In some embodiments, q is selected from 1 and 2. In some embodiments, q is 1.

In some embodiments, the compound or salt of Formula (A), (A-1), or (I) is represented by the structure of Formula (I-A):

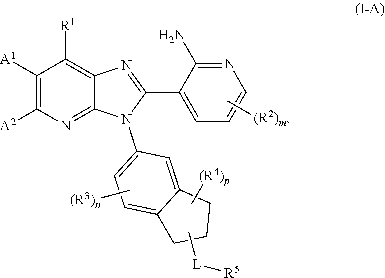

or a pharmaceutically acceptable salt thereof, wherein:
   R¹ is selected from hydrogen, halogen, C₁₋₄ alkyl, C₁₋₄ haloalkyl, —OR¹⁰, —SR¹⁰, —N(R¹⁰)₂, —NO₂, and —CN;
   A¹ and A² are each independently selected from:
      hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO 2, and —CN;
      C₁₋₆ alkyl, C₂₋₆ alkenyl, and C₂₋₆ alkynyl, any of which is optionally substituted with one or more substituents independently selected from:
         halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
      5- to 10-membered heterocycle and C₃₋₁₀ carbocycle, any of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹) 2, —NO₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
      C₃₋₁₀ carbocycle and 3- to 10-membered heterocycle; wherein the C₃₋₁₀ carbocycle and 3- to 10-membered heterocycle are each optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN; and
   m and n are each independently selected from 0, 1, 2, and 3;
   R² is independently selected at each instance from halogen, C₁₋₄ alkyl, C₁₋₄ haloalkyl, —OR¹², —SR¹², —N(R¹²)₂, —NO₂, and —CN;
   R³ is independently selected at each instance from:
      halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
      C₁₋₆ alkyl optionally substituted with one or more substituents independently selected from
         halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN;
   p is selected from 0, 1, 2, 3, 4, and 5;
   R⁴ is independently selected at each instance from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
      C₁₋₆ alkyl, C₂₋₆ alkenyl, C₂₋₆ alkynyl, any one of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
      two R⁴ attached to the same atom are taken together to form a group selected from:
         ═O, ═S, and ═N(R¹⁴); or
      two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from: halogen, C₁₋₄ alkyl, C₁₋₄ haloalkyl, —OR¹⁴, —SR¹⁴, —N(R¹⁴)₂, —NO₂, and —CN;
   L is represented by -L¹-L²-L³-L⁴-, wherein L¹, L², L³, and L⁴ are each independently selected from (a) and (b):
      (a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
      (b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
      wherein L², L³, and L⁴ are each optionally absent;
      wherein no more than two of L¹, L², L³, and L⁴ are selected from (a) and the two selected are not adjacent;
   R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from:
      halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
      4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN;
   R¹⁰, R¹¹, R¹², R¹³, R¹⁴, and R¹⁵ are each independently selected at each occurrence from:
      hydrogen, C₁₋₄ alkyl, C₃₋₈ carbocycle, 3- to 8-membered heterocycle, and C₁₋₄ haloalkyl;
   R¹⁶ is independently selected at each occurrence from:
      hydrogen;
      C₁₋₄ alkyl, wherein the C₁₋₄ alkyl is optionally substituted with one or more substituents independently selected from:
         halogen, —OR²⁰, —SR²⁰, —N(R²⁰)₂, —C(O)R²⁰, —C(O)OR²⁰, —OC(O)R²⁰, —C(O)N(R²⁰) 2, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN;
         C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
         C₃₋₈ carbocycle and 3- to 8-membered heterocycle, either of which is optionally substituted with one or more substituents independently selected from:
         halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —NO₂, and —CN; and
   R²⁰ is independently selected at each occurrence from hydrogen, C₁₋₄ alkyl, C₁₋₄ haloalkyl, C₃₋₈ carbocycle, and 3- to 8-membered heterocycle.

In some embodiments, the compound or salt of Formula (A), (A-1), (I), or (I-A) is represented by the structure of Formula (II):

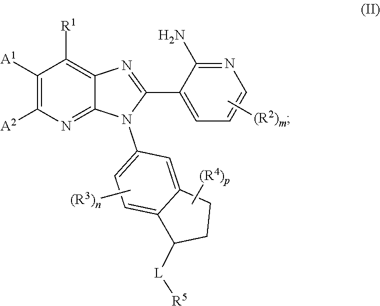

or a pharmaceutically acceptable salt thereof, wherein A¹, A², R¹, R², R³, R⁴, R⁵, m, n, p, and L are each defined as in Formula (A), (A-1), (I), or (I-A).

In some embodiments, the compound or salt of Formula (A), (A-1), (I), or (I-A) is represented by the structure of Formula (II-A):

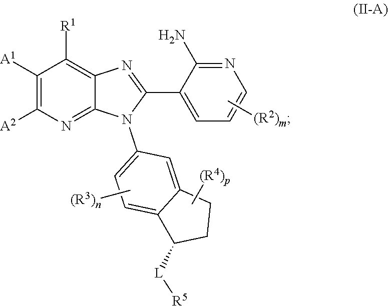

or a pharmaceutically acceptable salt thereof, wherein A¹, A², R¹, R², R³, R⁴, R⁵, m, n, p, and L are each defined as in Formula (A), (A-1), (I), or (I-A).

In some embodiments, the compound or salt of Formula (A), (A-1), (I), or (I-A) is represented by the structure of Formula (II-B):

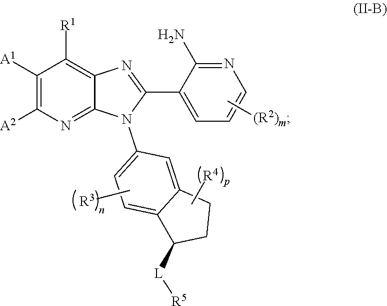

or a pharmaceutically acceptable salt thereof, wherein A¹, A², R¹, R², R³, R⁴, R⁵, m, n, p, and L are each defined as in Formula (A), (A-1), (I), or (I-A).

In some embodiments, for the compound or salt of Formula (A), (A-1), (I), (I-A), (II), (II-A), or (II-B), R¹ is selected from hydrogen, halogen, C₁₋₄ alkyl, C₁₋₄ haloalkyl, —OR¹⁰, —SR¹⁰. —N(R¹⁰)₂, —NO₂, and —CN.

In some embodiments, for the compound or salt of Formula (A), (A-1), (I), (I-A), (II), (II-A), or (II-B), R¹ is selected from: hydrogen, halogen, C₁₋₄ alkyl, C₁₋₄ haloalkyl, —OR¹⁰, and —CN. In some embodiments, R¹ is selected from fluoro, chloro, methyl, ethyl, propyl, isopropyl, n-butyl, sec-butyl, —CHF₂, —CH₂F, —CF₃, —OCH₃, —OCHF₂, —OCH₂F, —OCF₃, and —CN. In some embodiments, R¹ is selected from fluoro, chloro, methyl, ethyl, propyl, isopropyl, —CHF₂, —CF₃, —OCH₃, —OCHF₂, —OCF₃, and —CN. In some embodiments, R¹ is selected from fluoro, chloro, methyl, ethyl, propyl, isopropyl, —CHF₂, —OCH₃, —OCHF₂, and —CN.

In some embodiments, for the compound or salt of Formula (A), (A-1), (I), (I-A), (II), (II-A), or (II-B), R¹ is selected from:
hydrogen, halogen, C₁₋₄ alkyl, C₁₋₄ haloalkyl, —OR¹⁰, —SR¹⁰, —N(R¹⁰)₂, —NO₂, and —CN; and R¹⁰ is independently selected at each occurrence from hydrogen and C₁₋₄ alkyl.

In some embodiments, for the compound or salt of Formula (A), (A-1), (I), (I-A), (II), (II-A), or (II-B), R¹ is selected from hydrogen, halogen, C₁₋₄ alkyl, and C₁₋₄ haloalkyl. In some embodiments, R¹ is selected from hydrogen and C₁₋₄ alkyl. In some embodiments, R¹ is hydrogen. In some embodiments, R¹ is C₁₋₄ alkyl. In some embodiments, R¹ is selected from methyl, ethyl, propyl, and isopropyl.

In some embodiments, for the compound or salt of Formula (A), (A-1), (I), (I-A), (II), (II-A), or (II-B), R² is independently selected at each instance from halogen, C₁₋₄ alkyl, C₁₋₄ haloalkyl, —OR¹², —SR¹², —N(R¹²)₂, —NO₂, and —CN.

In some embodiments, for the compound or salt of Formula (A), (A-1), (I), (I-A), (II), (II-A), or (II-B), R² is independently selected at each instance from:
halogen, C₁₋₄ alkyl, C₁₋₄ haloalkyl, —OR¹², —SR¹², —N(R¹²)₂, —NO₂, and —CN; and
R¹² is independently selected at each occurrence from hydrogen and C₁₋₄ alkyl.

In some embodiments, for the compound or salt of Formula (A), (A-1), (I), (I-A), (II), (II-A), or (II-B), R² is independently selected at each instance from halogen, C₁₋₄ alkyl, and C₁₋₄ haloalkyl.

In some embodiments, for the compound or salt of Formula (A), (A-1), (I), (I-A), (II), (II-A), or (II-B), R² is independently selected at each instance from halogen, C₁₋₄ alkyl, and C₁₋₄ haloalkyl.

In some embodiments, for the compound or salt of Formula (A), (A-1), (I), (I-A), (II), (II-A), or (II-B), R² is independently selected at each instance from halogen and C₁₋₄ alkyl. In some embodiments, R² is independently selected at each instance from fluoro, chloro, methyl, ethyl, propyl, and isopropyl. In some embodiments, R² is independently selected at each instance from fluoro, methyl, and ethyl. In some embodiments, R² is independently selected at each instance from fluoro and methyl.

In some embodiments, for the compound or salt of Formula (A), (A-1), (I), (I-A), (II), (II-A), or (II-B), m is selected from 0, 1, 2, and 3. In some embodiments, m is selected from 0 and 1. In some embodiments, m is selected from 0. In some embodiments, m is 1.

In some embodiments, the compound or salt of Formula (A), (A-1), (I), or (I-A) is represented by the structure of Formula (III):

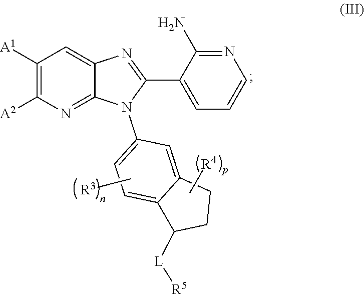

Or a pharmaceutically acceptable salt thereof, wherein A¹, A², R³, R⁴, R⁵, n, p, and L are each defined as in Formula (A), (A-1), (I), or (I-A).

In some embodiments, the compound or salt of Formula (A), (A-1), (I), or (I-A) is represented by the structure of Formula (III-A):

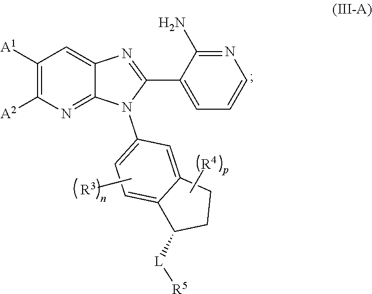

or a pharmaceutically acceptable salt thereof, wherein A¹, A², R³, R⁴, R⁵, n, p, and L are each defined as in Formula (A), (A-1), (I), or (I-A).

In some embodiments, the compound or salt of Formula (A), (A-1), (I), or (I-A) is represented by the structure of Formula (III-B):

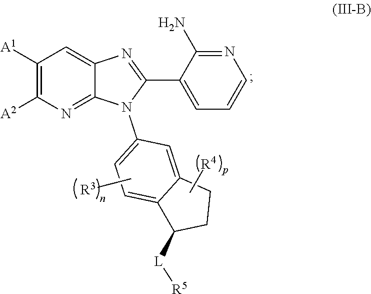

or a pharmaceutically acceptable salt thereof, wherein A¹, A², R³, R⁴, R⁵, n, p, and L are each defined as in Formula (A), (A-1), (I), or (I-A).

In some embodiments, for the compound or salt of Formula (A), (A-1), (I), (I-A), (II), (II-A), (II-B), (III), (III-A), or (III-B), A¹ and A² are each independently selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
5- to 10-membered heterocycle and C₃₋₁₀ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₃₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹)₂, —NO₂, ═O, ═S, ═N(R¹¹), and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
      C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN.

In some embodiments, for the compound or salt of Formula (A), (A-1), (I), (I-A), (II), (II-A), (II-B), (III), (III-A), or (III-B), A¹ and A² are each independently selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
5- to 10-membered heterocycle and C₃₋₁₀ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₃₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₃₋₁₀ carbocycle and 3- to 10-membered heterocycle; wherein the C₃₋₁₀ carbocycle and 3- to 10-membered heterocycle are each optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
   C₁₋₆ alkyl, C₂₋₆ alkenyl, and C₃₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN; and
R¹¹ is independently selected at each occurrence from hydrogen, C₁₋₄ alkyl, C₃₋₈ carbocycle, 3- to 8-membered heterocycle, and C₁₋₄ haloalkyl.

In some embodiments, for the compound or salt of Formula (A), (A-1), (I), (I-A), (II), (II-A), (II-B), (III), (III-A), or (III-B), A¹ and A² are each independently selected from:
hydrogen, halogen, C₁₋₄ haloalkyl, —OR¹¹, —N(R¹¹)₂, —C(O)N(R¹¹)₂, —N(R¹¹)C(O)R¹¹ and —CN;
C₁₋₆ alkyl, C₂₋₆ alkenyl, and C₂₋₆ alkynyl, any of which is optionally substituted with one or more substituents independently selected from:
   halogen, —OR¹¹, —N(R¹¹)₂, —C(O)N(R¹¹)₂, —N(R¹¹)C(O)R¹¹, ═O, and —CN; and
5- to 10-membered heterocycle and C₃₋₁₀ carbocycle, any of which is optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN;
   C₁₋₆ alkyl, C₂₋₆ alkenyl, and C₃₋₆ alkynyl, any one of which is optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, —NO₂, ═O, and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, ═O, and —CN; and
      C₁₋₆ alkyl, C₂₋₆ alkenyl, and C₃₋₆ alkynyl, any one of which is optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN.

In some embodiments, for the compound or salt of Formula (A), (A-1), (I), (I-A), (II), (II-A), (II-B), (III), (III-A), or (III-B), A¹ and A² are each independently selected from:
hydrogen, halogen, C₁₋₄ haloalkyl, —OR¹¹, —N(R¹¹)₂, and —CN;
C₁₋₆ alkyl and C₂₋₆ alkynyl, any of which is optionally substituted with one or more halogen; and
5- to 10-membered heterocycle and C₃₋₁₀ carbocycle, any of which is optionally substituted with one or more substituents independently selected from:
   halogen, —OR¹¹, C₁₋₆ alkyl, C₁₋₆ haloalkyl, C₃₋₁₀ carbocycle and 3- to 10-membered heterocycle; wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), or (III-B), A¹ and A² are each independently selected from:
hydrogen, halogen, —OR¹¹, C₁₋₆ alkyl and C₂₋₆ alkynyl; and
5- to 10-membered heterocycle and C₃₋₁₀ carbocycle, any of which is optionally substituted with one or more substituents independently selected from:
   halogen, C₁₋₆ alkyl, C₁₋₆ haloalkyl, and 3- to 10-membered heterocycle; wherein the 3- to 10-membered heterocycle is optionally substituted with one or more C₁₋₆ alkyl.

In some embodiments, for the compound or salt of Formula (A), (A-1), (I), (I-A), (II), (II-A), (II-B), (III), (III-A), or (III-B), A¹ and A² are each independently selected from:
hydrogen, halogen, —OR¹¹, C₁₋₆ alkyl and C₂₋₆ alkynyl; and
5- to 10-membered heterocycle and C₃₋₁₀ carbocycle, any of which is optionally substituted with one or more substituents independently selected from:
   halogen, C₁₋₆ alkyl, C₁₋₆ haloalkyl, and 3- to 10-membered heterocycle; wherein the 3- to 10-membered heterocycle is optionally substituted with one or more C₁₋₆ alkyl; and
R¹¹ is independently selected at each occurrence from hydrogen and C₁₋₄ alkyl.

In some embodiments, for the compound or salt of Formula (A), (A-1), (I), (I-A), (II), (II-A), (II-B), (III), (III-A), or (III-B), A¹ and A² are each independently selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

In some embodiments, for the compound or salt of Formula (A), (A-1), (I), (I-A), (II), (II-A), (II-B), (III), (III-A), or (III-B), A¹ and A² are each independently selected from:
hydrogen, halogen, C₁₋₄ haloalkyl, —OR¹¹, —N(R¹¹)₂, —C(O)N(R¹¹)₂, —N(R¹¹)C(O)R¹¹ and —CN; and
C₁₋₆ alkyl, C₂₋₆ alkenyl, and C₂₋₆ alkynyl, any of which is optionally substituted with one or more substituents independently selected from:
   halogen, —OR¹¹, —N(R¹¹)₂, —C(O)N(R¹¹)₂, —N(R¹¹)C(O)R¹¹, ═O, and —CN.

In some embodiments, for the compound or salt of Formula (A), (A-1), (I), (I-A), (II), (II-A), (II-B), (III), (III-A), or (III-B), A¹ and A² are each independently selected from hydrogen, halogen, C₁₋₄ haloalkyl, —OR¹¹, —N(R¹¹)₂, —CN, C₁₋₆ alkyl and C₂₋₆ alkynyl, any of which is optionally substituted with one or more halogen.

In some embodiments, for the compound or salt of Formula (A), (A-1), (I), (I-A), (II), (II-A), (II-B), (III), (III-A), or (III-B), A¹ and A² are each independently selected from hydrogen, halogen, —OR¹¹, C₁₋₆ alkyl, and C₂₋₄ alkynyl; and R¹¹ is selected from hydrogen, C₁₋₄ alkyl, C₁₋₄ haloalkyl, and C₃₋₆ carbocycle.

In some embodiments, for the compound or salt of Formula (A), (A-1), (I), (I-A), (II), (II-A), (II-B), (III), (III-A), or (III-B), A¹ and A² are each independently selected from hydrogen, halogen, C₁₋₆ alkyl, C₂₋₄ alkynyl, —OH, —OC₁₋₄ alkyl, and —OC₃₋₆ carbocycle.

In some embodiments, for the compound or salt of Formula (A), (A-1), (I), (I-A), (II), (II-A), (II-B), (III), (III-A), or (III-B), A¹ is selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
5- to 10-membered heterocycle and C₃₋₁₀ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₃₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
      C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN.

In some embodiments, for the compound or salt of Formula (A), (A-1), (I), (I-A), (II), (II-A), (II-B), (III), (III-A), or (III-B), A¹ is selected from:
hydrogen, halogen, C₁₋₄ haloalkyl, —OR¹¹, —N(R¹¹)₂, and —CN;
C₁₋₆ alkyl and C₂₋₆ alkynyl, any of which is optionally substituted with one or more halogen; and
5- to 10-membered heterocycle and C₃₋₁₀ carbocycle, any of which is optionally substituted with one or more substituents independently selected from:
   halogen, —OR¹¹, C₁₋₆ alkyl, C₁₋₆ haloalkyl, C₃₋₁₀ carbocycle and 3- to 10-membered heterocycle; wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), or (III-B), A¹ is selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂,
   —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), —CN.

In some embodiments, for the compound or salt of Formula (A), (A-1), (I), (I-A), (II), (II-A), (II-B), (III), (III-A), or (III-B), A¹ is selected from:
hydrogen, halogen, C₁₋₄ haloalkyl, —OR¹¹, —N(R¹¹)₂, —C(O)N(R¹¹)₂, —N(R¹¹)C(O)R¹¹ and —CN; and
C₁₋₆ alkyl, C₂₋₆ alkenyl, and C₂₋₆ alkynyl, any of which is optionally substituted with one or more substituents independently selected from:
   halogen, —OR¹¹, —N(R¹¹)₂, —C(O)N(R¹¹)₂, —N(R¹¹)C(O)R¹¹, ═O, and —CN.

In some embodiments, for the compound or salt of Formula (A), (A-1), (I), (I-A), (II), (II-A), (II-B), (III), (III-A), or (III-B), A¹ is selected from hydrogen, halogen, C₁₋₄ alkyl, C₁₋₄ haloalkyl, —OR¹¹, —N(R¹¹)₂ and —CN. In some embodiments, A¹ is selected from hydrogen, halogen, C₁₋₄ alkyl, and C₁₋₄ haloalkyl. In some embodiments, A¹ is selected from hydrogen, fluoro, and methyl. In some embodiments, A¹ is hydrogen.

In some embodiments, for the compound or salt of Formula (A), (A-1), (I), (I-A), (II), (II-A), (II-B), (III), (III-A), or (III-B), A¹ is selected from hydrogen, halogen, C₁₋₄ alkyl, C₁₋₄ haloalkyl, —OR¹¹, —N(R¹¹)₂ and —CN. In some embodiments, A¹ is selected from hydrogen, halogen, C₁₋₄ alkyl, C₁₋₄ haloalkyl, and —OR¹¹. In some embodiments, A¹ is selected from hydrogen, fluoro, methyl, —OH, and —OCH₃. In some embodiments, A¹ is selected from fluoro, methyl, —OH, and —OCH₃. In some embodiments, A¹ is hydrogen.

In some embodiments, for the compound or salt of Formula (A), (A-1), (I), (I-A), (II), (II-A), (II-B), (III), (III-A), or (III-B), A² is selected from:
hydrogen, halogen, C₁₋₄ haloalkyl, —OR¹¹, —N(R¹¹)₂, —C(O)N(R¹¹)₂, —N(R¹¹)C(O)R¹¹ and —CN;
C₁₋₆ alkyl, C₂₋₆ alkenyl, and C₂₋₆ alkynyl, any of which is optionally substituted with one or more substituents independently selected from: halogen, —OR¹¹, —N(R¹¹)₂, —C(O)N(R¹¹)₂, —N(R¹¹)C(O)R¹¹, ═O, and —CN; and
5- to 10-membered heterocycle and C₃₋₁₀ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN;
   C₁₋₆ alkyl, C₂₋₆ alkenyl, and C₃₋₆ alkynyl, any one of which is optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, —NO₂, ═O, and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from halogen, —OR¹¹, —N(R¹¹)₂, —C(O)R¹¹, —C(O)N(R¹¹)₂, —N(R¹¹)C(O)R¹¹, ═O, and —CN; and C₁₋₆ alkyl, C₂₋₆ alkenyl, and C₃₋₆ alkynyl, any one of which is optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN.

In some embodiments, for the compound or salt of Formula (A), (A-1), (I), (I-A), (II), (II-A), (II-B), (III), (III-A), or (III-B), A² is selected from:
hydrogen, halogen, C₁₋₄ haloalkyl, —OR¹¹, —N(R¹¹)₂, and —CN;
C₁₋₆ alkyl and C₂₋₆ alkynyl, any of which is optionally substituted with one or more halogen; and
5- to 10-membered heterocycle and C₃₋₁₀ carbocycle, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₁₋₆ haloalkyl, C₃₋₁₀ carbocycle, and 3- to 10-membered heterocycle; wherein the C₁₋₆ alkyl are each optionally substituted with one or more substituents selected from halogen, —OR¹¹, or —N(R¹¹)₂; wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), or (III-B), A² is selected from: hydrogen, fluoro, bromo, chloro, methyl, ethyl, ethynylene, trifluoromethyl,

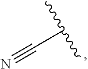 [SMILES: *C#N]

—OCH₃, cyclopropyl,

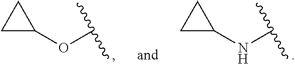 [SMILES: *OC1CC1 ; *NC1CC1]

In some embodiments, for the compound or salt of Formula (A), (A-1), (I), (I-A), (II), (II-A), (II-B), (III), (III-A), or (III-B), A² is selected from 5- to 10-membered heterocycle and C₃₋₁₀ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN;
C₁₋₆ alkyl, C₂₋₆ alkenyl, and C₃₋₆ alkynyl, any one of which is optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹,
   —N(R¹¹)S(O)₂R¹¹, —NO₂, ═O, and —CN; and
C₃₋₁₀ carbocycle and 3- to 10-membered heterocycle; wherein the C₃₋₁₀ carbocycle and 3- to 10-membered heterocycle are each optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, ═O, and —CN; and
   C₁₋₆ alkyl, C₂₋₆ alkenyl, and C₃₋₆ alkynyl, any one of which is optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN.

In some embodiments, for the compound or salt of Formula (A), (A-1), (I), (I-A), (II), (II-A), (II-B), (III), (III-A), or (III-B), A² is selected from 5- to 10-membered heterocycle and C₃₋₁₀ carbocycle, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₁₋₆ haloalkyl, C₃₋₁₀ carbocycle, and 3- to 10-membered heterocycle; wherein C₁₋₆ alkyl and C₃₋₆ alkynyl are each optionally substituted with one or more substituents independently selected from halogen, —OR¹¹, and —N(R¹¹)₂; and wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), or (III-B), A² is selected from 5- to 10-membered heterocycle and C₃₋₁₀ carbocycle, any of which is optionally substituted with one or more substituents independently selected from: fluoro, chloro, methyl, —CHF₂, —CF₃, ethynylene, —OCH₃, —SCH₃, —NH₂, —N(CH₃)₂, —C(O)H, —CN, —CH₂OCH₃, —CH₂OH, cyclopropyl, pyrazolyl, azetidinyl, and N-methylpiperazinyl, wherein azetidinyl is optionally substituted with methyl.

In some embodiments, for the compound or salt of Formula (A), (A-1), (I), (I-A), (II), (II-A), (II-B), (III), (III-A), or (III-B), A² is selected from: cyclopropyl, phenyl, morpholinyl, piperazinyl, pyrazolyl, pyridinyl, pyridazinyl, pyrazinyl, pyrimidinyl, triazolyl, oxazolyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₃₋₁₀ carbocycle, and 3- to 6-membered heterocycle; wherein the C₁₋₆ alkyl is optionally substituted with one or more substituents independently selected from halogen and —OR¹¹, wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), or (III-B), A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₃₋₁₀ carbocycle, and 3- to 6-membered heterocycle; wherein the C₁₋₆ alkyl is optionally substituted with one or more substituents independently selected from halogen and —OR¹¹, wherein the C₃₋₁₀ carbocycle and 3- to 10-membered heterocycle are each optionally substituted with one or more C₁₋₆ alkyl. In some embodiments, A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₃₋₈ carbocycle, and 3- to 6-membered heterocycle; wherein the C₁₋₆ alkyl is optionally substituted with one or more substituents independently selected from halogen and —OR¹¹, wherein the C₃₋₈ carbocycle and 3- to 10-membered heterocycle are each optionally substituted with one or more C₁₋₆ alkyl. In some embodiments, A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₃₋₆ carbocycle, and 3- to 6-membered heterocycle; wherein the C₁₋₆ alkyl is optionally substituted with one or more substituents independently selected from halogen and —OR¹¹, wherein the C₃₋₆ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), or (III-B), A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₁₋₆ haloalkyl, and 3- to 10-membered heterocycle optionally substituted with one or more C₁₋₄ alkyl. In some embodiments, A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₁₋₆ haloalkyl, and 3- to 6-membered heterocycle optionally substituted with one or more C₁₋₄ alkyl. In some embodiments, A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: halogen, —OR¹¹, —N(R¹¹)₂, —CN, C₁₋₆ alkyl, C₃₋₆ alkynyl, C₁₋₆ haloalkyl, and 3- to 6-membered heterocycle optionally substituted with one or more C₁₋₄ alkyl. In some embodiments, A² is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: fluoro, —OR¹¹, —CN, C₁₋₆ alkyl, C₃₋₆ alkynyl, C₁₋₆ haloalkyl, and 3- to 6-membered heterocycle optionally substituted with one or more C₁₋₄ alkyl.

In some embodiments, for the compound or salt of Formula (A), (A-1), (I), (I-A), (II), (II-A), (II-B), (III), (III-A), or (III-B), A² is selected from: cyclopropyl, phenyl, morpholinyl, piperazinyl, pyrazolyl, pyridinyl, pyridazinyl, pyrazinyl, pyrimidinyl, triazolyl, oxazolyl, and thiazolyl, any of which is optionally substituted with one or more substituents independently selected from: halogen, —OR¹¹, —SR¹¹, —N(R¹¹)₂, —C(O)R¹¹, —CN, C₁₋₆ alkyl, C₃₋₆ alkynyl, C₁₋₆ haloalkyl, and 3- to 6-membered heterocycle optionally substituted with one or more C₁₋₄ alkyl.

In some embodiments, for the compound or salt of Formula (A), (A-1), (I), (I-A), (II), (II-A), (II-B), (III), (III-A), or (III-B), A² is selected from: cyclopropyl, phenyl, morpholinyl, piperazinyl, pyrazolyl, pyridinyl, pyridazinyl, pyrazinyl, pyrimidinyl, triazolyl, oxazolyl, and thiazolyl, any of which is optionally substituted with one or more substituents independently selected from: fluoro, chloro, methyl, and N-methylpiperazinyl.

In some embodiments, for the compound or salt of Formula (A), (A-1), (I), (I-A), (II), (II-A), (II-B), (III), (III-A), or (III-B), A² is selected from is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: fluoro, chloro, methyl, and N-methylpiperazinyl. In some embodiments, A² is selected from is selected from: cyclopropyl, phenyl, morpholinyl, piperazinyl, pyrazolyl, pyridinyl, pyridazinyl, pyrazinyl, pyrimidinyl, triazolyl, oxazolyl, imidazolyl, oxadiazolyl, thiadiazolyl, pyrrolopyrazolyl, furopyrazolyl, pyrazolopyridinyl, pyrazolooxazinyl, and thiazolyl, any of which is optionally substituted with one or more substituents independently selected from: fluoro, methyl, and N-methylpiperazinyl.

In some embodiments, for the compound or salt of Formula (A), (A-1), (I), (I-A), (II), (II-A), (II-B), (III), (III-A), or (III-B), A² is pyrazolyl optionally substituted with one or more oxteanyl, pyrazolyl, and azetidinyl, wherein the oxteanyl, pyrazolyl, and azetidinyl are each optionally substituted with C₁₋₆ alkyl. In some embodiments, A² is pyrazolyl optionally substituted with one or more oxteanyl, pyrazolyl, and azetidinyl, wherein the oxteanyl, pyrazolyl, and azetidinyl are each optionally substituted with methyl, ethyl, propyl, isopropyl, and n-butyl. In some embodiments, A² is pyrazolyl optionally substituted with one or more oxteanyl, pyrazolyl, and azetidinyl, wherein the oxteanyl, pyrazolyl, and azetidinyl are each optionally substituted with methyl and ethyl. In some embodiments, A² is pyrazolyl optionally substituted with one or more oxteanyl, pyrazolyl, and azetidinyl, wherein the oxteanyl, pyrazolyl, and azetidinyl are each optionally substituted with methyl.

In some embodiments, for the compound or salt of Formula (A), (A-1), (I), (I-A), (II), (II-A), (II-B), (III), (III-A), or (III-B), A² is pyrazolyl optionally substituted with one or more substituents independently selected from
halogen, —CN, —C(O)R¹¹, —N(R¹¹)₂, —SR¹¹;
C₁₋₆ alkyl, C₃₋₆ alkynyl, C₁₋₆ haloalkyl, —OC₁₋₆ alkyl, —OC₁₋₆ haloalkyl; and
C₃₋₁₀ carbocycle and 3- to 10-membered heterocycle optionally substituted with —OR¹¹ and C₁₋₆ alkyl.

In some embodiments, for the compound or salt of Formula (A), (A-1), (I), (I-A), (II), (II-A), (II-B), (III), (III-A), or (III-B), A² is pyrazolyl optionally substituted with one or more C₃₋₁₀ carbocycle. In some embodiments, A² is pyrazolyl optionally substituted with one or more cyclopropyl.

In some embodiments, for the compound or salt of Formula (A), (A-1), (I), (I-A), (II), (II-A), (II-B), (III), (III-A), or (III-B), A² is pyrazolyl optionally substituted with one or more 3- to 6-membered heterocycle optionally substituted with one or more C₁₋₆ alkyl. In some embodiments, A² is pyrazolyl optionally substituted with one or more pyrazolyl and azetidinyl, wherein the pyrazolyl and azetidinyl are each optionally substituted with C₁₋₆ alkyl. In some embodiments, A² is pyrazolyl optionally substituted with one or more pyrazolyl and azetidinyl, wherein the pyrazolyl and azetidinyl are each optionally substituted with methyl.

In some embodiments, for the compound or salt of Formula (A), (A-1), (I), (I-A), (II), (II-A), (II-B), (III), (III-A), or (III-B), A² is selected from: —F, —Cl, —CH₃, —CN, cyclopropyl, ethynylene, —CF₃, phenyl,

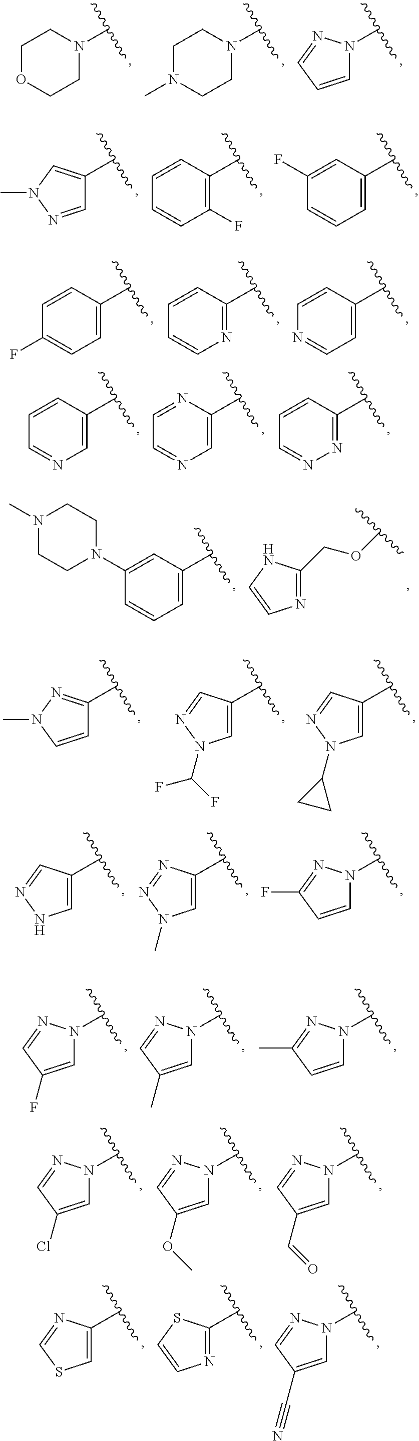

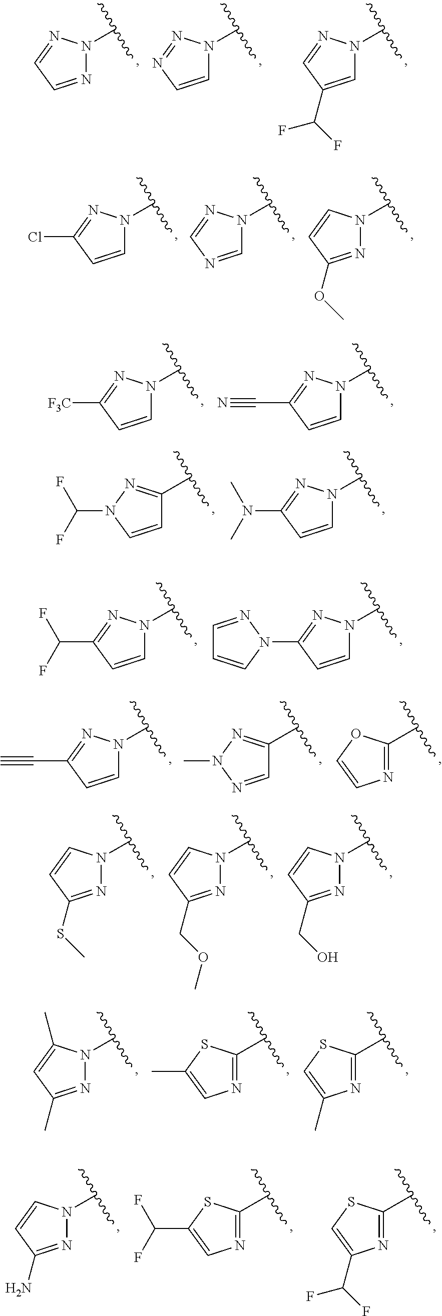

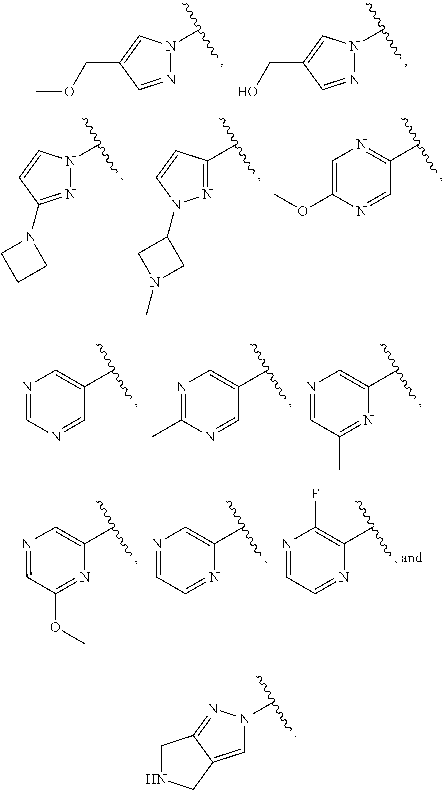

In some embodiments, for the compound or salt of Formula (A), (A-1), (I), (I-A), (II), (II-A), (II-B), (III), (III-A), or (III-B), A² is selected from: cyclopropyl, phenyl,

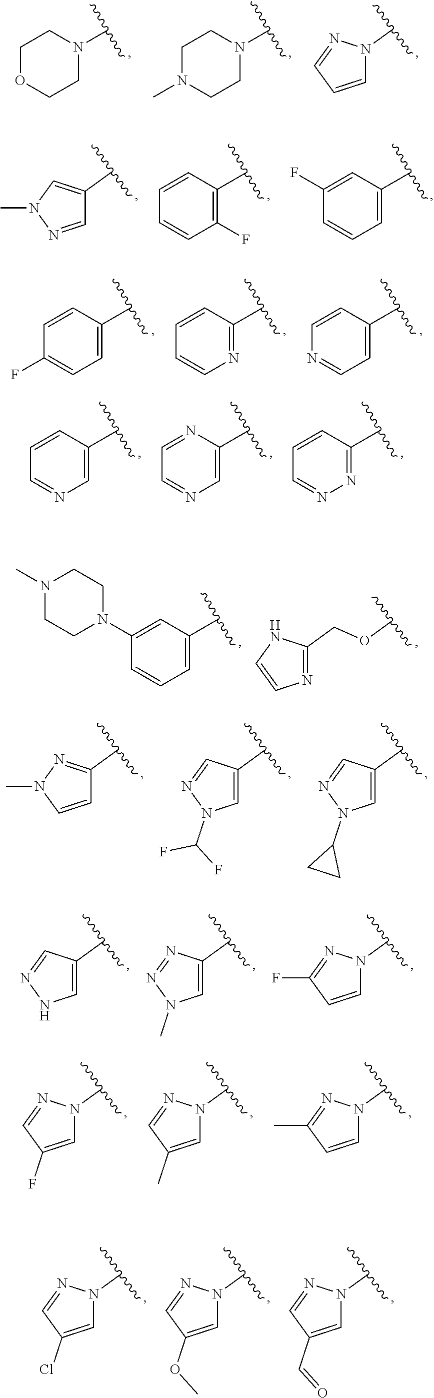

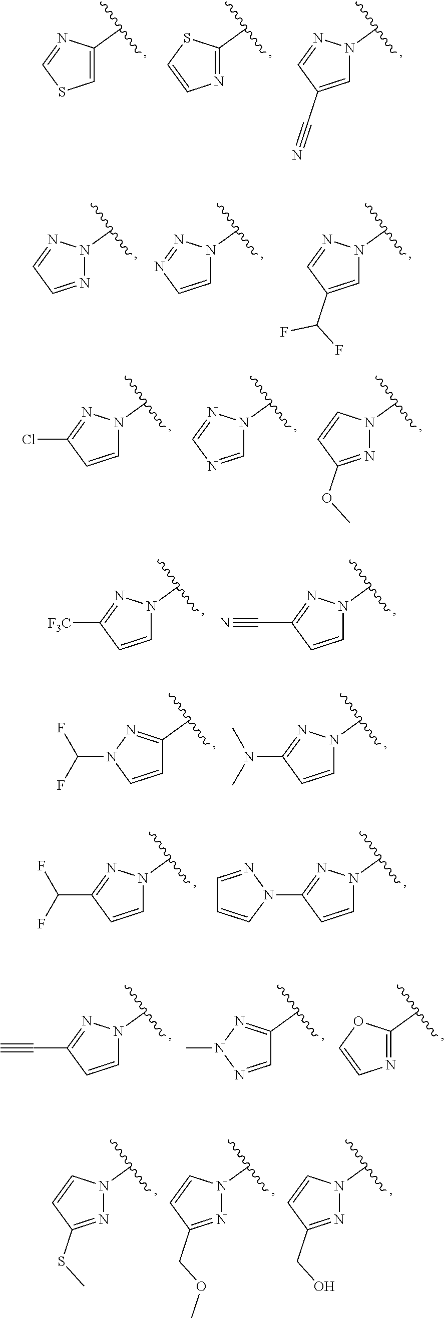

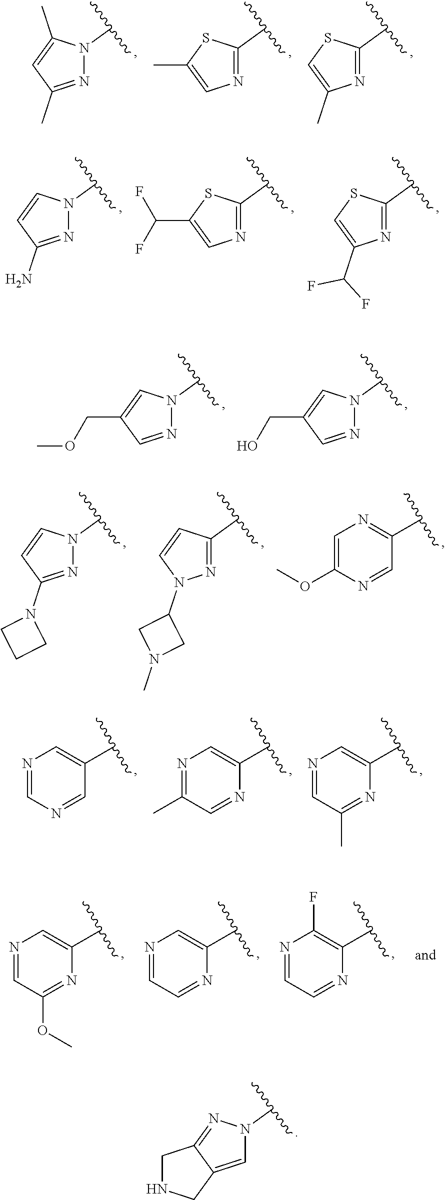

In some embodiments, for the compound or salt of Formula (A), (A-1), (I), (I-A), (II), (II-A), (II-B), (III), (III-A), or (III-B), A² is selected from: —F, —Cl, —CH₃, —CN, cyclopropyl, ethynylene, —CF₃, phenyl,

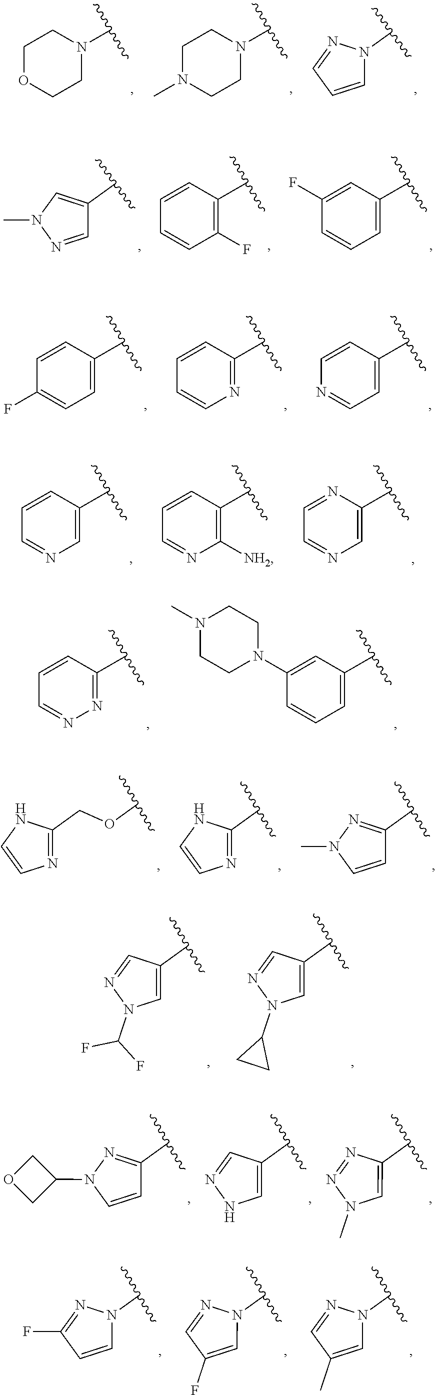

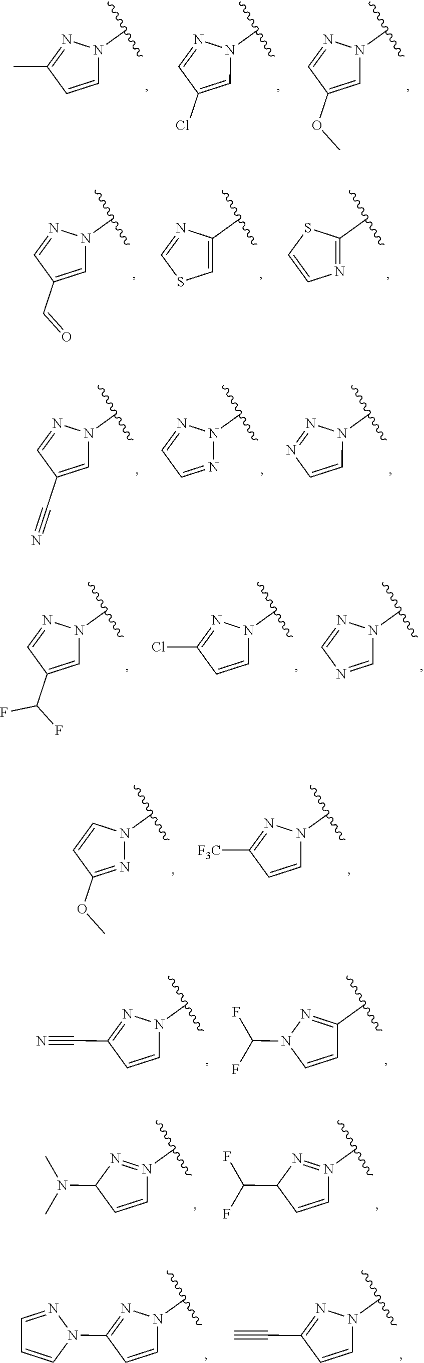

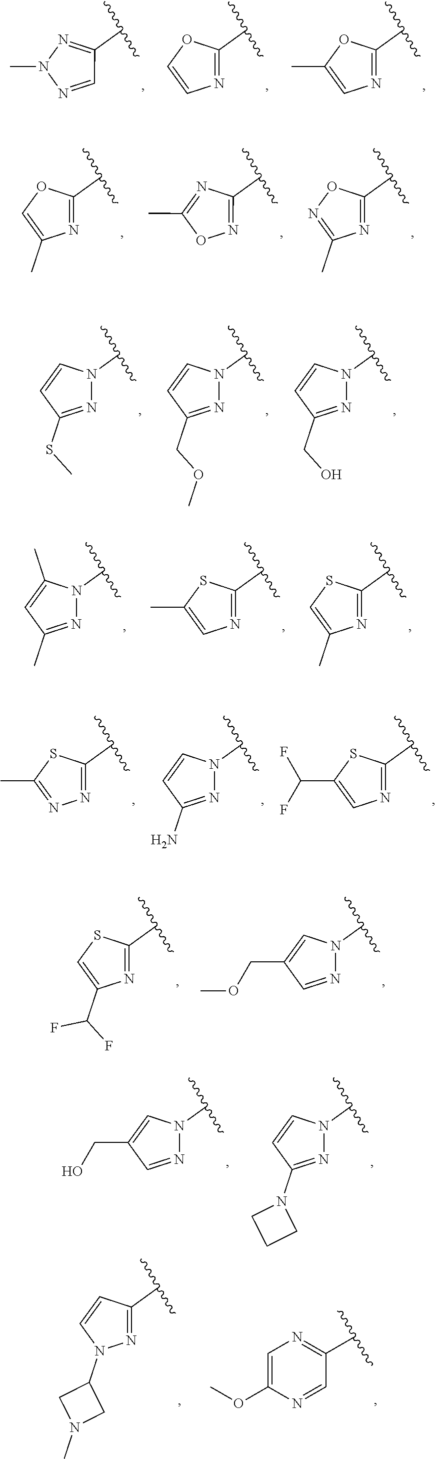

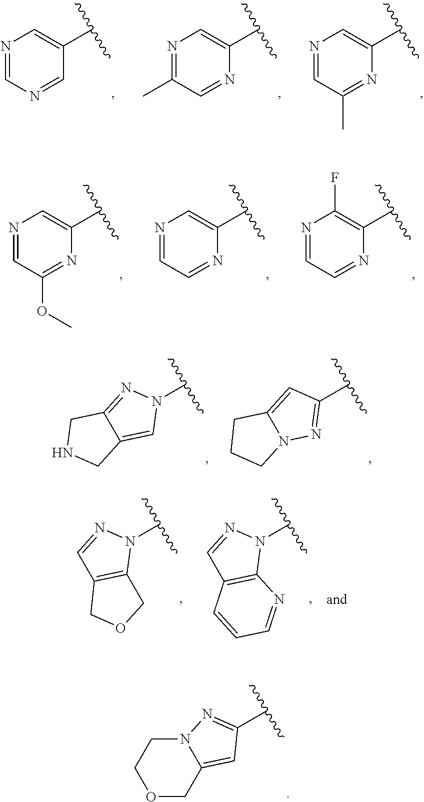

In some embodiments, the compound or salt of Formula (A), (A-1), (I), or (I-A) is represented by the structure of Formula (IV):

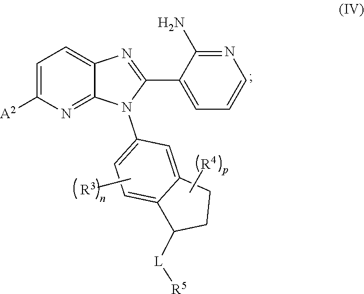

or a pharmaceutically acceptable salt thereof, wherein A², R³, R⁴, R⁵, n, p, and L are each defined as in Formula (A), (A-1), (I), or (I-A).

In some embodiments, the compound or salt of Formula (A), (A-1), (I), or (I-A) is represented by the structure of Formula (IV-A):

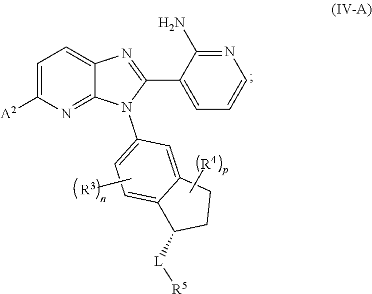

or a pharmaceutically acceptable salt thereof, wherein A², R³, R⁴, R⁵, n, p, and L are each defined as in Formula (A), (A-1), (I), or (I-A).

In some embodiments, the compound or salt of Formula (A), (A-1), (I), or (I-A) is represented by the structure of Formula (IV-B):

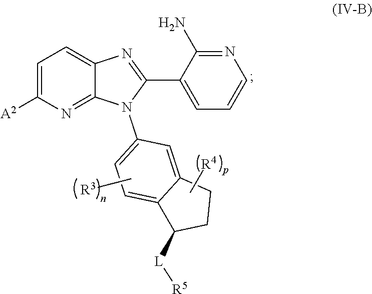

or a pharmaceutically acceptable salt thereof, wherein A², R³, R⁴, R⁵, n, p, and L are each defined as in Formula (A), (A-1), (I), or (I-A).

In some embodiments, for the compound or salt of Formula (A), (A-1), (I), (I-A), (II), (II-A), (II-B), (III), (III-A), (III-B), (IV), (IV-A), or (IV-B), R³ is independently selected at each instance from:
halogen, —OR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
C₁₋₆ alkyl optionally substituted with one or more substituents independently selected from:
   halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN.

In some embodiments, for the compound or salt of Formula (A), (A-1), (I), (I-A), (II), (II-A), (II-B), (III), (III-A), (III-B), (IV), (IV-A), or (IV-B), R³ is independently selected at each instance from:
halogen, —OR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
C₁₋₆ alkyl optionally substituted with one or more substituents independently selected from:
   halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN; and
R¹³ is independently selected at each occurrence from hydrogen and C₁₋₄ alkyl, C₃₋₈ carbocycle, 3- to 8-membered heterocycle, and C₁₋₄ haloalkyl.

In some embodiments, for the compound or salt of Formula (A), (A-1), (I), (I-A), (II), (II-A), (II-B), (III), (III-A), (III-B), (IV), (IV-A), or (IV-B), R³ is independently selected at each instance from:
halogen, —OR¹³, —SR¹³, —N(R¹³)₂, —C(O)R¹³, —C(O)N(R¹³)₂, —N(R¹³)C(O)R¹³, and —CN; and
C₁₋₆ alkyl optionally substituted with one or more substituents independently selected from:
   halogen, —OR¹³, —N(R¹³)₂, —C(O)R¹³, —C(O)N(R¹³)₂, —N(R¹³)C(O)R¹³, ═O, and —CN.

In some embodiments, for the compound or salt of Formula (A), (A-1), (I), (I-A), (II), (II-A), (II-B), (III), (III-A), (III-B), (IV), (IV-A), or (IV-B), R³ is independently selected at each instance from halogen, —OR¹³, —N(R¹³)₂, —CN, C₁₋₆ alkyl, and C₁₋₆ haloalkyl.

In some embodiments, for the compound or salt of Formula (A), (A-1), (I), (I-A), (II), (II-A), (II-B), (III), (III-A), (III-B), (IV), (IV-A), or (IV-B), R³ is independently selected at each instance from halogen, —OR¹³, —N(R¹³)₂, —CN, C₁₋₆ alkyl, and C₁₋₆ haloalkyl; and R¹³ is independently selected at each occurrence from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), or (IV-B), R³ is independently selected at each instance from halogen, C₁₋₄ alkyl C₁₋₄haloalkyl and —CN

In some embodiments, for the compound or salt of Formula (A), (A-1), (I), (I-A), (II), (II-A), (II-B), (III), (III-A), (III-B), (IV), (IV-A), or (IV-B), n is selected from 0, 1, 2, and 3. In some embodiments, n is selected from 0 and 1. In some embodiments, n is 0.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from:
halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
C₁₋₆ alkyl, C₂₋₆ alkenyl, C₂₋₆ alkynyl, any one of which is optionally substituted with one or more substituents selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
two R⁴ attached to the same atom are taken together to form a group selected from:
   ═O, ═S, and ═N(R¹⁴); or
two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from halogen, C₁₋₄ alkyl, C₁₋₄ haloalkyl, —OR¹⁴, —SR¹⁴, —N(R¹⁴)₂, —NO₂, and —CN.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from:
halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
C₁₋₆ alkyl, C₂₋₆ alkenyl, C₂₋₆ alkynyl, any one of which is optionally substituted with one or more substituents selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
two R⁴ attached to the same atom are taken together to form a group selected from:
   ═O, ═S, and ═N(R¹⁴); or
two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from halogen, C₁₋₄ alkyl, C₁₋₄ haloalkyl, —OR¹⁴, —SR¹⁴, —N(R¹⁴)₂, —NO₂, and —CN; and
R¹⁴ is independently selected at each occurrence from hydrogen, C₁₋₄ alkyl, C₃₋₈ carbocycle, 3- to 8-membered heterocycle, and C₁₋₄ haloalkyl.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from:
halogen, —OR¹⁴, —N(R¹⁴)₂, —C(O)R¹⁴, —C(O)N(R¹⁴)₂, and —CN; and
C₁₋₆ alkyl, C₂₋₆ alkenyl, C₂₋₆ alkynyl, any one of which is optionally substituted with one or more substituents selected from halogen, —OR¹⁴, —N(R¹⁴)₂, —C(O)R¹⁴, —C(O)N(R¹⁴)₂, —N(R¹⁴)C(O)R¹⁴, ═O, and —CN; or
two R⁴ attached to the same atom are taken together to form a group selected from:
   ═O, ═S, and ═N(R¹⁴); or
two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from halogen, C₁₋₄ alkyl, C₁₋₄ haloalkyl, —OR¹⁴, —N(R¹⁴)₂, and —CN.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from:
halogen, —OR¹⁴, —SR¹⁴, —N(R¹⁴)₂, —CN, C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents selected from halogen, —OR¹⁴, —N(R¹⁴)₂, ═O, and —CN; or
two R⁴ attached to the same atom are taken together to form ═O; or
two R⁴ attached to the same atom or to adjacent atoms are taken together with the carbons to which they are attached to form a group selected from 3- to 8-membered heterocycle and C₃₋₈ carbocycle; and
R¹⁴ is selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from:
halogen, —OR¹⁴, C₁₋₆ alkyl, and C₂₋₆ alkynyl; or
two R⁴ attached to the same atom are taken together to form ═O; or
two R⁴ attached to the same atom or to adjacent atoms are taken together with the carbons to which they are attached to form a group selected from 3- to 8-membered heterocycle and C₃₋₈ carbocycle; and
R¹⁴ is selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from:
halogen, —OH, C₁₋₆ alkyl, and C₂₋₆ alkynyl; or
two R⁴ attached to the same atom are taken together to form ═O; or
two R⁴ attached to the same atom or to adjacent atoms are taken together with the carbons to which they are attached to form a C₃₋₈ carbocycle.

In some embodiments, for the compound or salt of Formula (A), (A-1), (I), (I-A), (II), (II-A), (II-B), (III), (III-A), (III-B), (IV), (IV-A), or (IV-B), R⁴ is independently selected at each instance from: halogen, —OH, C₁₋₆ alkyl, and C₂₋₆ alkynyl; or two R⁴ attached to the same atom or to adjacent atoms are taken together with the carbons to which they are attached to form a C₃₋₆ carbocycle. In some embodiments, R⁴ is independently selected at each instance from: halogen, —OH, and C₁₋₆ alkyl; or two R⁴ attached to the same atom or to adjacent atoms are taken together with the carbons to which they are attached to form a C₃₋₆ carbocycle. In some embodiments, R⁴ is independently selected at each instance from halogen and C₁₋₆ alkyl; or two R⁴ attached to the same atom or to adjacent atoms are taken together with the carbons to which they are attached to form a C₃₋₆ carbocycle. In some embodiments, R⁴ is independently selected at each instance from fluoro, chloro, methyl, ethyl, propyl, isopropyl, and n-butyl; or two R⁴ attached to the same atom or to adjacent atoms are taken together with the carbons to which they are attached to form a C₃₋₆ carbocycle. In some embodiments, R⁴ is independently selected at each instance from fluoro and methyl; or two R⁴ attached to the same atom or to adjacent atoms are taken together with the carbons to which they are attached to form cyclopropyl.

In some embodiments, for the compound or salt of Formula (A), (A-1), (I), (I-A), (II), (II-A), (II-B), (III), (III-A), (III-B), (IV), (IV-A), or (IV-B), p is selected from 0, 1, 2, 3, 4, and 5. In some embodiments, p is selected from 0, 1, and 2. In some embodiments, p is selected from 0, and 1. In some embodiments, p is 0.

In some embodiments, the compound or salt of Formula (A), (A-1), (I), or (I-A) is represented by the structure of Formula (V):

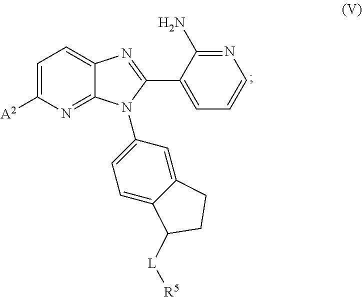

or a pharmaceutically acceptable salt thereof, wherein A², R⁵, and L are each defined as in Formula (A), (A-1), (I), or (I-A).

In some embodiments, the compound or salt of Formula (A), (A-1), (I), or (I-A) is represented by the structure of Formula (V-A):

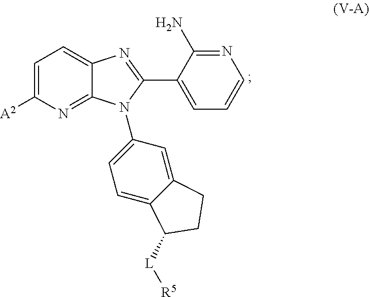

or a pharmaceutically acceptable salt thereof, wherein A², R⁵, and L are each defined as in Formula (A), (A-1), (I), or (I-A).

In some embodiments, the compound or salt of Formula (A), (A-1), (I), or (I-A) is represented by the structure of Formula (V-B):

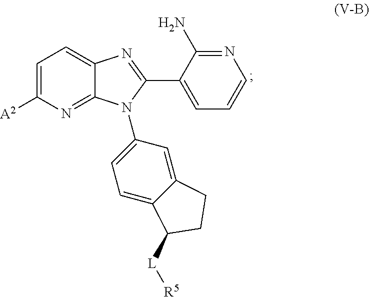

or a pharmaceutically acceptable salt thereof, wherein A², R⁵, and L are each defined as in Formula (A), (A-1), (I), or (I-A).

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L¹, L², L³, and L⁴ are each independently selected from (a) and (b):
(a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O) N(R¹⁵)—, and —(R¹⁵)NC(O)N(R¹⁵)N(R¹⁵)—; and
(b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
wherein L², L³, and L⁴ are each optionally absent; and
wherein no more than two of L¹, L², L³, and L⁴ are selected from (a) and the two selected are not adjacent.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L¹, L², L³, and L⁴ are each independently selected from (a) and (b):
(a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O) N(R¹⁵)—, and —(R¹⁵)NC(O)N(R¹⁵)N(R¹⁵)—; and
(b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN; and
R¹⁵ is independently selected at each occurrence from hydrogen and C₁₋₄ alkyl, C₃₋₈ carbocycle, 3- to 8-membered heterocycle, and C₁₋₄ haloalkyl;
wherein L², L³, and L⁴ are each optionally absent; and
wherein no more than two of L¹, L², L³, and L⁴ are selected from (a) and the two selected are not adjacent

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L¹, L², L³, and L⁴ are each independently selected from (a) and (b):
(a) —O—, —N(R¹⁵)—, —S—, —N(R¹⁵)C(O)—, —N(R¹⁵)C(O)O—, —N(R¹⁵)S(O)₂—N(R¹⁵)N(R¹⁵)—, and —(R¹⁵)NC(O)N(R¹⁵)—; and
(b) C₁₋₆ alkylene, C₂₋₆ alkenylene, C₂₋₆ alkynylene, C₃₋₆ carbocyclene, and 3- to 6-membered heterocyclene, any of which is optionally substituted with one or more substituents independently selected from halogen, —OR¹⁵, ═O, and —CN;
wherein L², L³, and L⁴ are each optionally absent; and
wherein no more than two of L¹, L², L³, and L⁴ are selected from (a) and the two selected are not adjacent.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L¹, L², L³, and L⁴ are each independently selected from (a) and (b):
(a) —N(R¹⁵)— and —N(R¹⁵)C(O)—; and
(b) C₁₋₆ alkylene, C₂₋₆ alkynylene, and C₃₋₆ carbocyclene;
wherein L², L³, and L⁴ are each optionally absent;
wherein no more than two of L¹, L², L³, and L⁴ are selected from (a) and the two selected are not adjacent; and
R¹⁵ is selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L¹, L², L³, and L⁴ are each independently selected from (a) and (b):
(a) —N(R¹⁵)— and —N(R¹⁵)C(O)—; and
(b) C₁₋₆ alkylene and C₃₋₆ carbocyclene;
wherein L², L³, and L⁴ are each optionally absent;
wherein no more than two of L¹, L², L³, and L⁴ are selected from (a) and the two selected are not adjacent; and
R¹⁵ is selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L¹, L², L³, and L⁴ are each independently selected from (a) and (b):
(a) —NH— and —N(H)C(O)—; and
(b) methylene,

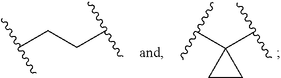

wherein L², L³, and L⁴ are each optionally absent; and
wherein no more than two of L¹, L², L³, and L⁴ are selected from (a) and the two selected are not adjacent.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L², L³, and L⁴ are absent; and L¹ is selected from:
(a) —O— —N(R¹⁵)—, —S—, —S(O)—, —S(O)₂—, —S(O)(NR¹⁵)—, —N(R¹⁵)C(O)—, —N(R¹¹)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
(b) C₁₋₆ alkylene, C₂₋₆ alkenylene, C₂₋₆ alkynylene.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L², L³, and L⁴ are absent; and L¹ is selected from:
(a) —O— —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
(b) C₁₋₆ alkylene, C₂₋₆ alkenylene, C₂₋₆ alkynylene; and
R¹⁵ is selected from hydrogen and C₁₋₄ alkyl, and C₁₋₄ haloalkyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L², L³, and L⁴ are absent; and L¹ is selected from —N(R¹⁵)— and —N(R¹⁵)C(O)—, and C₁₋₆ alkylene.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L², L³, and L⁴ are absent; and L¹ is selected from:
—N(R¹⁵)— and —N(R¹⁵)C(O)—, and C₁₋₆ alkylene; and
R¹⁵ is selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L², L³, and L⁴ are absent; and L¹ is selected from:
—N(R¹⁵)— and —N(R¹⁵)C(O)—, and C₁₋₆ alkylene; and
R¹⁵ is selected from hydrogen and methyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L², L³, and L⁴ are absent; and L¹ is selected from —N(H)—, —N(H)C(O)—, and C₁₋₆ alkylene. In some embodiments, L², L³, and L⁴ are absent;
and L¹ is selected from —NH— and —N(H)C(O)—.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L³ and L⁴ are absent; and
L¹ is selected from —O— —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹¹)N(R¹¹)—, —N(R¹⁵)N(R¹⁵)—, —(R¹⁵)NC(O)N(R¹⁵)—, and —(R¹⁵)NC(O)N(R¹⁵)N(R¹⁵)—; and
L² is selected from C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L³ and L⁴ are absent; and
L¹ is selected from —O— —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹¹)N(R¹¹)—, —N(R¹⁵)N(R¹⁵)—, —(R¹⁵)NC(O)N(R¹⁵)—, and —(R¹⁵)NC(O)N(R¹⁵)N(R¹⁵)—; and
L² is selected from C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN; and
R¹⁵ is independently selected at each occurrence from hydrogen and C₁₋₄ alkyl, and C₁₋₄ haloalkyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L³ and L⁴ are absent; and
L¹ is selected from —N(R¹⁵)— and —N(R¹⁵)C(O)—; and
L² is selected from C₁₋₆ alkylene, C₂₋₆ alkenylene, C₃₋₈ carbocyclene.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L³ and L⁴ are absent; and
L¹ is selected from —N(R¹⁵)— and —N(R¹⁵)C(O)—; and
L² is selected from C₁₋₆ alkylene and C₃₋₆ carbocyclene.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L³ and L⁴ are absent; and
L¹ is selected from —N(R¹⁵)— and —N(R¹⁵)C(O)—;
L² is selected from C₁₋₆ alkylene and C₃₋₆ carbocyclene; and
R¹⁵ is selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is represented by -L¹-L²-L³-L⁴-, wherein L³ and L⁴ are absent; and
L¹ is selected from —N(H)— and —N(H)C(O)—; and
L² is selected from methylene,

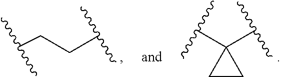

In some embodiments, for the compound or salt of Formula (A), (A-1), (I), (I-A), (II), (II-A), (II-B), (III), (III-A), (II-B), (IV), (IV-A), (IV-B), (V), (V-A or V-B), L is selected from —N(H)—, —N(H)C(O)—,

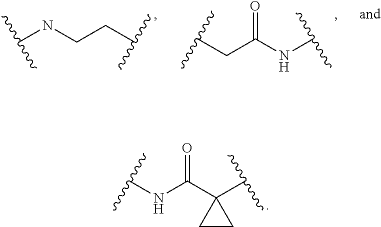 [SMILES: *CCN* ; *CC(=O)N* ; *NC(=O)C1(*)CC1]

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is selected from —N(H)— and —N(H)C(O)—.

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is selected from

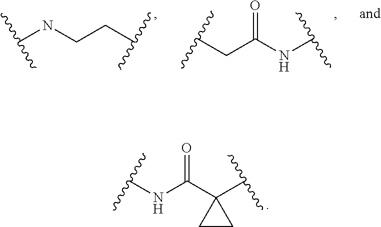 [SMILES: *CCN* ; *CC(=O)N* ; *NC(=O)C1(*)CC1]

In some embodiments, for the compound or salt of Formula (A), (A-1), (I), (I-A), (II), (II-A), (II-B), (III), (III-A), (III-B), (IV), (IV-A), (IV-B), (V), (V-A), or (V-B), L is selected from

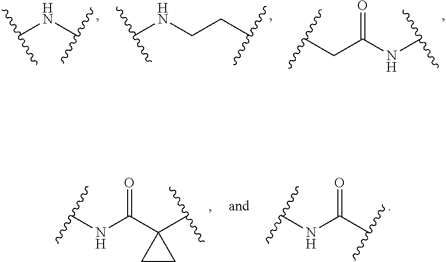

In some embodiments, the compound or salt of Formula (A), (A-1), (I), or (I-A) is represented by the structure of Formula (VI):

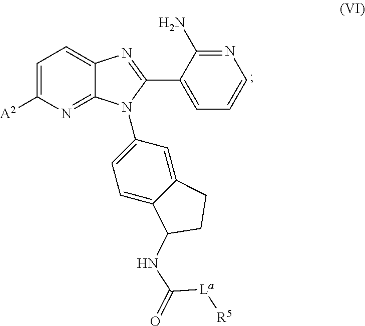

or a pharmaceutically acceptable salt thereof, wherein L^{a} is selected from:
a direct bond; and
C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
   and wherein A² and R⁵ are each defined as in Formula (A), (A-1), (I), or (I-A).

In some embodiments, the compound or salt of Formula (A), (A-1), (I), or (I-A) is represented by the structure of Formula (VI-A):

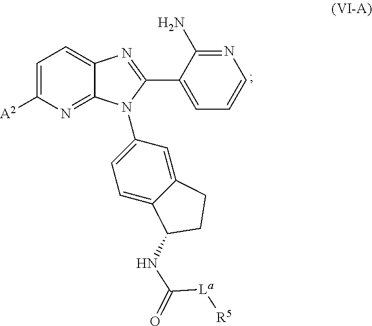

or a pharmaceutically acceptable salt thereof, wherein A² and R⁵ are each defined as in Formula (A), (A-1), (I), or (I-A), and L^{a} is defined as in Formula (VI).

In some embodiments, the compound or salt of Formula (I) is represented by the structure of Formula (VI-B):

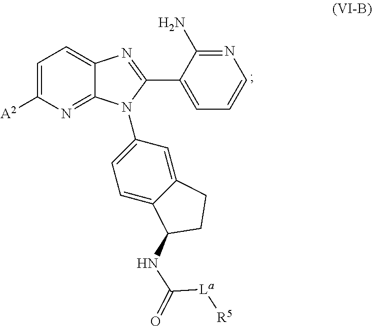

or a pharmaceutically acceptable salt thereof, wherein A² and R⁵ are each defined as in Formula (A), (A-1), (I), or (I-A), and L^{a} is defined as in Formula (VI).

In some embodiments, the compound or salt of Formula (A), (A-1), (I), or (I-A) is represented by the structure of Formula (VII):

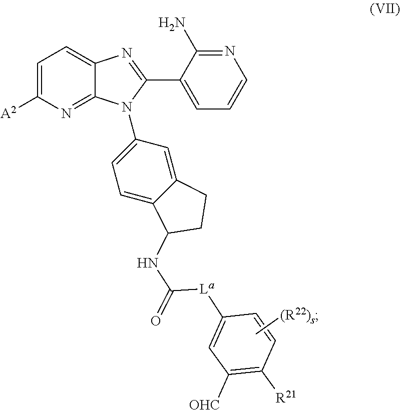

or a pharmaceutically acceptable salt thereof, wherein:
   R²¹ is selected from:
      hydrogen, halogen, C₁₋₄ alkyl, C₁₋₄
         haloalkyl, —OR¹⁶, —SR¹⁶, —N(R¹⁶)₂, —B(OR¹⁶)₂, —C(O)R¹⁶, —C(O)N(R¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN; and
      4- to 6-membered heterocycle optionally substituted with one or more substituents independently selected from halogen, C₁₋₄ alkyl, C₁₋₄ haloalkyl, —OR¹⁶, and —CN; and
   R²² is independently selected at each instance from:
      halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, and —CN; and
      4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN; and
   s is selected from 0, 1, 2, and 3;
and wherein A² is defined as in Formula (A), (A-1), (I), or (I-A), and L^{a} is defined as in Formula (VI).

In some embodiments, the compound or salt of Formula (A), (A-1), (I), or (I-A) is represented by the structure of Formula (VII-A):

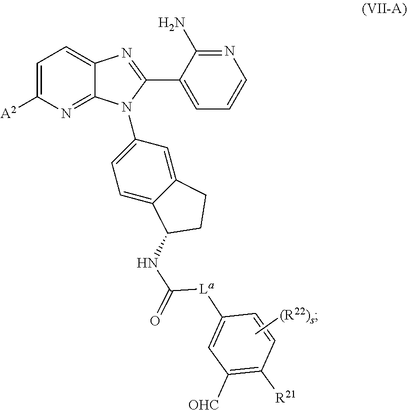

or a pharmaceutically acceptable salt thereof, wherein A² is defined as in Formula (A), (A-1), (I), or (I-A), L^{a} is defined as in Formula (VI), and R²¹, R²², and s are each defined as in Formula (VII).

In some embodiments, the compound or salt of Formula (A), (A-1), (I), or (I-A) is represented by the structure of Formula (VII-B):

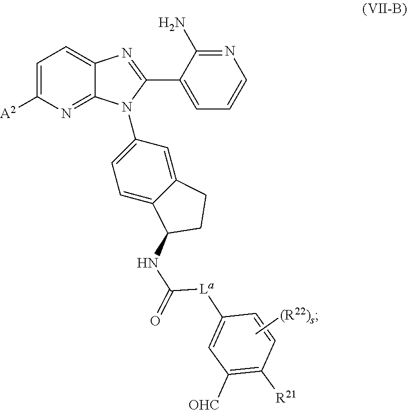

or a pharmaceutically acceptable salt thereof, wherein A² is defined as in Formula (A), (A-1), (I), or (I-A), L^{a} is defined as in Formula (VI), and R²¹, R²², and s are each defined as in Formula (VII).

In some embodiments, the compound or salt of Formula (A), (A-1), (I), or (I-A) is represented by the structure of Formula (VIII):

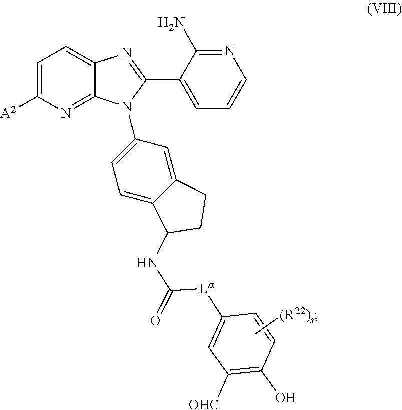

or a pharmaceutically acceptable salt thereof, wherein A² is defined as in Formula (A), (A-1), (I), or (I-A), L^{a} is defined as in Formula (VI), and R²² and s are each defined as in Formula (VII).

In some embodiments, the compound or salt of Formula (A), (A-1), (I), or (I-A) is represented by the structure of Formula (VIII-A):

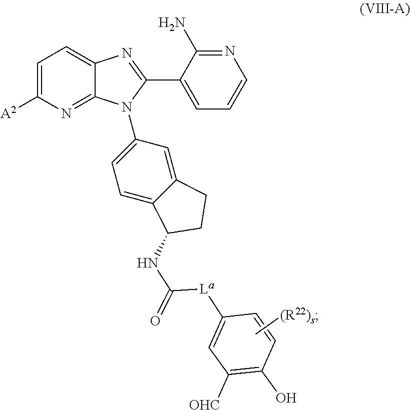

or a pharmaceutically acceptable salt thereof, wherein A is defined as in Formula (A), (A-1), (I), or (I-A), L^{a} is defined as in Formula (VI), and R²² and s are each defined as in Formula (VII).

In some embodiments, the compound or salt of Formula (A), (A-1), (I), or (I-A) is represented by the structure of Formula (VIII-B):

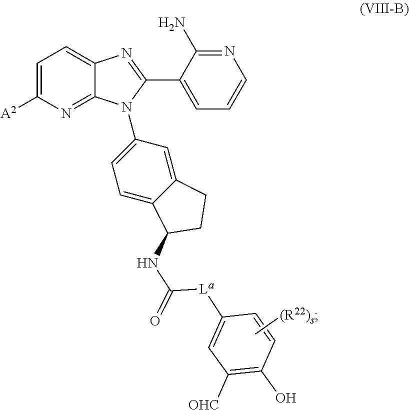

or a pharmaceutically acceptable salt thereof, wherein A² is defined as in Formula (A), (A-1), (I), or (I-A), L^{a} is defined as in Formula (VI), and R²² and s are each defined as in Formula (VII).

In some embodiments, for the compound or salt of Formula (VI), (VI-A), (VI-B), (VII), (VII-A), (VII-B), (VIII), (VIII-A), or (VIII-B), L^{a} is selected from:
a direct bond; and
C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN.

In some embodiments, for the compound or salt of Formula (VI), (VI-A), (VI-B), (VII), (VII-A), (VII-B), (VIII), (VIII-A), or (VIII-B), L^{a} is selected from:
a direct bond; and
C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN; and
R¹⁵ is independently selected at each occurrence from hydrogen and C₁₋₄ alkyl, C₃₋₈ carbocycle, 3- to 8-membered heterocycle, and C₁₋₄ haloalkyl.

In some embodiments, for the compound or salt of Formula (VI), (VI-A), (VI-B), (VII), (VII-A), (VII-B), (VIII), (VIII-A), or (VIII-B), L^{a} is selected from:
a direct bond; and
C₁₋₆ alkylene, C₂₋₆ alkenylene, C₂₋₆ alkynylene, C₃₋₆ carbocyclene, and 3- to 6-membered heterocyclene, any of which is optionally substituted with one or more substituents independently selected from halogen, —OR¹⁵, ═O, and —CN.

In some embodiments, for the compound or salt of Formula (VI), (VI-A), (VI-B), (VII), (VII-A), (VII-B), (VIII), (VIII-A), or (VIII-B), L^{a} is selected from:
a direct bond; and
C₁₋₆ alkylene, C₂₋₆ alkenylene, C₂₋₆ alkynylene, C₃₋₆ carbocyclene, and 3- to 6-membered heterocyclene, any of which is optionally substituted with one or more substituents independently selected from halogen, —OR¹⁵, ═O, and —CN; and
R¹⁵ is independently selected at each occurrence from hydrogen and C₁₋₄ alkyl, and C₁₋₄ haloalkyl.

In some embodiments, for the compound or salt of Formula (VI), (VI-A), (VI-B), (VII), (VII-A), (VII-B), (VIII), (VIII-A), or (VIII-B), L^{a} is selected from:
a direct bond; and
C₁₋₆ alkylene, C₂₋₆ alkynylene, and C₃₋₆ carbocyclene.

In some embodiments, for the compound or salt of Formula (VI), (VI-A), (VI-B), (VII), (VII-A), (VII-B), (VIII), (VIII-A), or (VIII-B), L^{a} is selected from:
a direct bond; and
methylene,

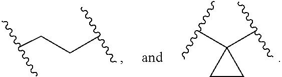

In some embodiments, the compound or salt of Formula (A), (A-1), (I), or (I-A) is represented by the structure of Formula (IX):

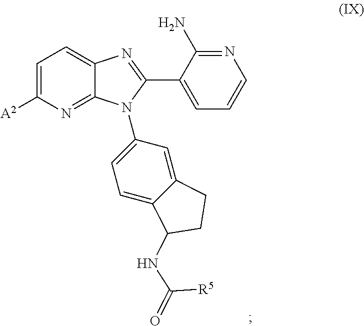

or a pharmaceutically acceptable salt thereof, wherein A² and R⁵ are each defined as in Formula (A), (A-1), (I), or (I-A).

In some embodiments, the compound or salt of Formula (A), (A-1), (I), or (I-A) is represented by the structure of Formula (IX-A):

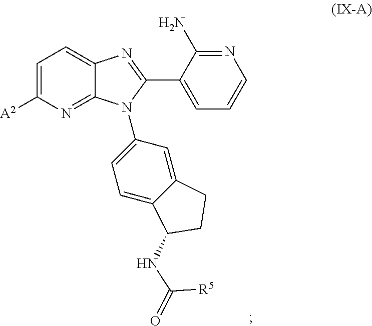

or a pharmaceutically acceptable salt thereof, wherein A² and R⁵ are each defined as in Formula (A), (A-1), (I), or (I-A).

In some embodiments, the compound or salt of Formula (A), (A-1), (I), or (I-A) is represented by the structure of Formula (IX-B):

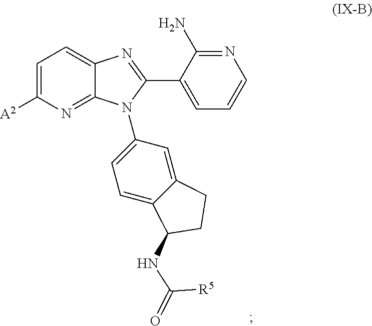

or a pharmaceutically acceptable salt thereof, wherein A² and R⁵ are each defined as in Formula (A), (A-1), (I), or (I-A).

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, ═O, and —CN; and
4- to 6-membered heterocycle optionally substituted with one or more substituents independently selected from halogen, C₁₋₄ alkyl, C₁₋₄ haloalkyl, —OR¹⁶, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H; and wherein R⁵ is further optionally substituted by one or more substituents independently selected from: halogen, C₁₋₄ alkyl, C₁₋₄ haloalkyl, —OR¹⁶, —C(O)R¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —CN, and 4- to 6-membered heterocycle. In some embodiments, R⁵ is selected from 3- to 10-membered heterocycle and C₃₋₁₀ carbocycle, any of which is substituted by —C(O)H; and wherein R⁵ is further optionally substituted by one or more substituents independently selected from: halogen, C₁₋₄ alkyl, C₁₋₄ haloalkyl, —OR¹⁶, —C(O)R¹⁶, —SR¹⁶, —N(R¹⁶)₂, —N(R¹⁶)S(O)₂R¹⁶, —S(O)₂N(R¹⁶)₂, —CN, and 4- to 6-membered heterocycle. In some embodiments, R⁵ is selected from 3- to 10-membered heterocycle and C₃₋₁₀ carbocycle, any of which is substituted by —C(O)H; and wherein R⁵ is further optionally substituted by one or more substituents independently selected from: halogen, C₁₋₄ alkyl, C₁₋₄ haloalkyl, —OR¹⁶, —N(R¹⁶)₂, —N(R¹⁶)S(O)₂R¹⁶, —S(O)₂N(R¹⁶)₂, —CN, and 4- to 6-membered heterocycle. In some embodiments, R⁵ is selected from 3- to 10-membered heterocycle and C₃₋₁₀ carbocycle, any of which is substituted by —C(O)H; and wherein R⁵ is further optionally substituted by one or more substituents independently selected from: fluoro, chloro, C₁₋₄ alkyl, C₁₋₄ haloalkyl, —OH, —OCH₃, —N(R¹⁶)₂, —N(R¹⁶)S(O)₂R¹⁶, —S(O)₂N(R¹⁶)₂, —CN, and 4- to 6-membered heterocycle. In some embodiments, R⁵ is selected from 3- to 10-membered heterocycle and C₃₋₁₀ carbocycle, any of which is substituted by —C(O)H; and wherein R⁵ is further optionally substituted by one or more substituents independently selected from: fluoro, C₁₋₄ alkyl, C₁₋₄ haloalkyl, —OH, —OCH₃, —CN, and 4- to 6-membered heterocycle.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with C₁₋₄ alkyl. In some embodiments, R⁵ is selected is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —C(O)R¹⁶, —S(O)₂N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with C₁₋₄ alkyl. In some embodiments, R⁵ is selected is selected from 3- to 10-membered heteroaryl and phenyl, any of which is substituted by —C(O)H or —C(O)D; and wherein R⁵ is further optionally substituted by one or more substituents independently selected from: fluoro, chloro, C₁₋₄ alkyl, C₂₋₆ alkynyl, C₁₋₄ haloalkyl, —OR¹⁶, —N(R¹⁶)₂, —CN, —S(O)₂N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with C₁₋₄ alkyl. In some embodiments, R⁵ is selected is selected from 3- to 10-membered heteroaryl and phenyl, any of which is substituted by —C(O)H or —C(O)D; and wherein R⁵ is further optionally substituted by one or more substituents independently selected from: fluoro, chloro, C₁₋₄ alkyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with methyl. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —C(O)R¹⁶, —N(R¹⁶)S(O)₂R¹⁶, —S(O)₂N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with methyl. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with methyl. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with methyl. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: fluoro, chloro, C₁₋₄ alkyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with methyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with halogen. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —C(O)R¹⁶, —N(R¹⁶)S(O)₂R¹⁶, —S(O)₂N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with halogen. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with halogen. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with halogen. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —B(OR¹⁶)₂, —CN, —C(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro or chloro. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —CN, —N(R¹⁶)C(O)R¹⁶, —N(R¹⁶)S(O)₂R¹⁶, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro or chloro. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro or chloro. In some embodiments, R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —CN, and 4- to 6-membered heterocycle; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro or chloro.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H; and wherein R⁵ is further optionally substituted by one or more substituents independently selected from: halogen, C₁₋₄ alkyl, —OR¹⁶, —N(R¹⁶)₂, —B(OR¹⁶)₂, —CN, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₆ alkyl, and C₃₋₆ cycloalkyl. In some embodiments, is selected from 3- to 10-membered heteroaryl and phenyl, any of which is substituted by —C(O)H; and wherein R⁵ is further optionally substituted by one or more substituents independently selected from: halogen, C₁₋₄ alkyl, —OR¹⁶, —N(R¹⁶)₂, —CN, —N(R¹⁶)C(O)R¹⁶, —N(R¹⁶)S(O)₂R¹⁶, and 4- to 6-membered heterocycle; and R¹⁶ is selected from hydrogen, C₁₋₆ alkyl, and C₃₋₆ cycloalkyl. In some embodiments, is selected from 3- to 10-membered heteroaryl and phenyl, any of which is substituted by —C(O)H; and wherein R⁵ is further optionally substituted by one or more substituents independently selected from: halogen, C₁₋₄ alkyl, —OR¹⁶, —N(R¹⁶)₂, —CN, and 4- to 6-membered heterocycle; and R¹⁶ is selected from hydrogen, C₁₋₆ alkyl, and C₃₋₆ cycloalkyl. In some embodiments, is selected from 3- to 10-membered heteroaryl and phenyl, any of which is substituted by —C(O)H; and wherein R⁵ is further optionally substituted by one or more substituents independently selected from: fluoro, chloro, C₁₋₄ alkyl, —OR¹⁶, —CN, and 4- to 6-membered heterocycle; and R¹⁶ is selected from hydrogen, C₁₋₆ alkyl, and C₃₋₆ cycloalkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —B(OR¹⁶)₂, —CN, —C(O)R¹⁶, —N(R¹⁶)C(O)R¹⁶, —N(R¹⁶)S(O)₂R¹⁶, —N(R¹⁶)C(O)N(R¹⁶)₂, and 4- to 6-membered heterocycle; and R¹⁶ is selected from hydrogen, methyl, —CHF₂, -morpholinyl, imidazolyl, and cyclopropyl, wherein the imidazolyl is optionally substituted with C₁₋₄ alkyl. In some embodiments, is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —CN, —C(O)R¹⁶, —N(R¹⁶)C(O)R¹⁶, —N(R¹⁶)S(O)₂R¹⁶, and 4- to 6-membered heterocycle; and R¹⁶ is selected from hydrogen, methyl, —CHF₂, -morpholinyl, imidazolyl, and cyclopropyl, wherein the imidazolyl is optionally substituted with C₁₋₄ alkyl. In some embodiments, is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —CN, and 4- to 6-membered heterocycle; and R¹⁶ is selected from hydrogen, methyl, —CHF₂, -morpholinyl, imidazolyl, and cyclopropyl, wherein the imidazolyl is optionally substituted with C₁₋₄ alkyl. In some embodiments, is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —CN, and 4- to 6-membered heterocycle; and R¹⁶ is selected from hydrogen, methyl, —CHF₂, -morpholinyl, imidazolyl, and cyclopropyl, wherein the imidazolyl is optionally substituted with C₁₋₄ alkyl. In some embodiments, is selected from 3- to 10-membered heteroaryl and phenyl, any of which is substituted by —C(O)H or —C(O)D; and wherein R⁵ is further optionally substituted by one or more substituents independently selected from: fluoro, chloro, C₁₋₄ alkyl, C₃₋₆ alkynyl, C₁₋₄ haloalkyl, —OR¹⁶, —CN, and 4- to 6-membered heterocycle; and R¹⁶ is selected from hydrogen, methyl, —CHF₂, -morpholinyl, imidazolyl, and cyclopropyl, wherein the imidazolyl is optionally substituted with C₁₋₄ alkyl. In some embodiments, is selected from 3- to 10-membered heteroaryl and phenyl, any of which is substituted by —C(O)H or —C(O)D; and wherein R⁵ is further optionally substituted by one or more substituents independently selected from: fluoro, C₁₋₄ alkyl, C₁₋₄ haloalkyl, —OR¹⁶, —CN, and 4- to 6-membered heterocycle; and R¹⁶ is selected from hydrogen, methyl, —CHF₂, -morpholinyl, imidazolyl, and cyclopropyl, wherein the imidazolyl is optionally substituted with C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H or —C(O)D, wherein R⁵ is further optionally substituted by one or more substituents independently selected from halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, and —N(R¹⁶)C(O)N(R¹⁶)₂, and —CN. In some embodiments, R⁵ is selected from 3- to 10-membered heterocycle and C₃₋₁₀ carbocycle, any of which is substituted by —C(O)H, wherein R⁵ is further optionally substituted by one or more substituents independently selected from halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H, wherein R⁵ is further optionally substituted by one or more substituents independently selected from halogen, C₁₋₄ alkyl, —OR¹⁶, and —N(R¹⁶)C(O)R¹⁶. In some embodiments, for the compound or salt of Formula (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H, wherein R⁵ is further optionally substituted by one or more substituents independently selected from halogen, methyl, —OH, and —N(R¹⁶)C(O)R¹⁶.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and C₃₋₁₀ carbocycle, any of which is substituted by —C(O)H, wherein R⁵ is further optionally substituted by one or more substituents independently selected from fluoro, chloro, —OH, methyl,

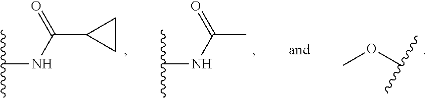

In some embodiments, R⁵ is phenyl substituted by —C(O)H, wherein R⁵ is further optionally substituted by one or more substituents independently selected from fluoro, chloro, —OH, methyl,

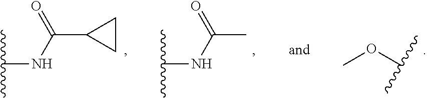

In some embodiments, R⁵ is phenyl substituted by —C(O)H and —OH, wherein R⁵ is further optionally substituted by one or more substituents independently selected from fluoro, chloro, —OH, methyl,

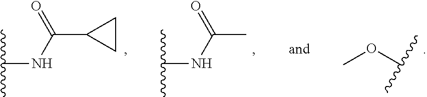

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and phenyl, any of which is substituted by —C(O)H or —C(O)D, wherein R⁵ is further optionally substituted by one or more substituents independently selected from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶,
   —NO₂, ═O, ═S, ═N(R¹⁶), and —CN; and
4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and phenyl, any of which is substituted by —C(O)H or —C(O)D, wherein R⁵ is further optionally substituted by one or more substituents independently selected from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, ═O, and —CN; and
4- to 6-membered heterocycle optionally substituted with one or more substituents independently selected from halogen, C₁₋₄ alkyl, C₁₋₄ haloalkyl, —OR¹⁶, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and phenyl, any of which is substituted by —C(O)H or —C(O)D, wherein R⁵ is further optionally substituted by one or more substituents independently selected from halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN. In some embodiments, R⁵ is selected from 3- to 10-membered heterocycle and phenyl, any of which is substituted by —C(O)H, wherein R⁵ is further optionally substituted by one or more substituents independently selected from halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and phenyl, any of which is substituted by —C(O)H, wherein R⁵ is further optionally substituted by one or more substituents independently selected from halogen, C₁₋₄ alkyl, —OR¹⁶, and —N(R¹⁶)C(O)R¹⁶. In some embodiments, R⁵ is selected from 3- to 10-membered heterocycle and phenyl, any of which is substituted by —C(O)H, wherein R⁵ is further optionally substituted by one or more substituents independently selected from halogen, methyl, —OH, and —N(R¹⁶)C(O)R¹⁶.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heterocycle and phenyl, any of which is substituted by —C(O)H, wherein R⁵ is further optionally substituted by one or more substituents independently selected from fluoro, chloro, —OH, methyl,

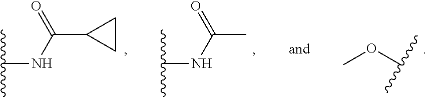

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —B(OR¹⁶)₂, —CN, —C(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₄ alkyl, and C₃₋₆ cycloalkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —CN, —C(O)R¹⁶, —N(R¹⁶)C(O)R¹⁶, —N(R¹⁶)S(O)₂R¹⁶, —S(O)₂N(R¹⁶)₂, and 4- to 6-membered heterocycle; and R¹⁶ is selected from hydrogen, C₁₋₄ alkyl, and C₃₋₆ cycloalkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —CN, and 4- to 6-membered heterocycle; and R¹⁶ is selected from hydrogen, C₁₋₄ alkyl, and C₃₋₆ cycloalkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —CN, and 4- to 6-membered heterocycle; and R¹⁶ is selected from hydrogen, C₁₋₄ alkyl, and C₃₋₆ cycloalkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —CN, and 4- to 6-membered heterocycle; and R¹⁶ is selected from hydrogen, C₁₋₄ alkyl, and C₃₋₆ cycloalkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —CN, and 4- to 6-membered heterocycle; and R¹⁶ is selected from hydrogen, methyl, cyclopropyl, cyclobutyl, and cyclopentyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, benzothiazole, benzoxazole, pyrazolopyridine, benzimidazole, and phenyl, any of which is substituted by —C(O)H; and wherein R⁵ is further optionally substituted by one or more substituents independently selected from: halogen, C₁₋₄ alkyl, —OR¹⁶, —N(R¹⁶)₂, —B(OR¹⁶)₂, —CN, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and 4- to 6-membered heterocycle; and R¹⁶ is selected from hydrogen, C₁₋₄ alkyl, and C₃₋₆ cycloalkyl. In some embodiments, R⁵ is selected from pyridine, isoquinoline, indazole, benzothiazole, benzoxazole, pyrazolopyridine, benzimidazole, and phenyl, any of which is substituted by —C(O)H; and wherein R⁵ is further optionally substituted by one or more substituents independently selected from: halogen, C₁₋₄ alkyl, —OR¹⁶, —N(R¹⁶)₂, —CN, —N(R¹⁶)C(O)R¹⁶, —N(R¹⁶)S(O)₂R¹⁶, —S(O)₂N(R¹⁶)₂, and 4- to 6-membered heterocycle; and R¹⁶ is selected from hydrogen, C₁₋₄ alkyl, and C₃₋₆ cycloalkyl. In some embodiments, R⁵ is selected from pyridine, isoquinoline, indazole, benzothiazole, benzoxazole, pyrazolopyridine, benzimidazole, and phenyl, any of which is substituted by —C(O)H; and wherein R⁵ is further optionally substituted by one or more substituents independently selected from: halogen, C₁₋₄ alkyl, —OR¹⁶, —N(R¹⁶)₂, —CN, and 4- to 6-membered heterocycle; and R¹⁶ is selected from hydrogen, C₁₋₄ alkyl, and C₃₋₆ cycloalkyl. In some embodiments, R⁵ is selected from pyridine, isoquinoline, indazole, benzothiazole, benzoxazole, pyrazolopyridine, benzimidazole, and phenyl, any of which is substituted by —C(O)H; and wherein R⁵ is further optionally substituted by one or more substituents independently selected from: halogen, C₁₋₄ alkyl, —OR¹⁶, —CN, and 4- to 6-membered heterocycle; and R¹⁶ is selected from hydrogen, C₁₋₄ alkyl, and C₃₋₆ cycloalkyl. In some embodiments, R⁵ is selected from pyridine, isoquinoline, indazole, benzothiazole, benzoxazole, pyrazolopyridine, benzimidazole, and phenyl, any of which is substituted by —C(O)H; and wherein R⁵ is further optionally substituted by one or more substituents independently selected from: fluoro, chloro, C₁₋₄ alkyl, —OR¹⁶, —CN, and 4- to 6-membered heterocycle; and R¹⁶ is selected from hydrogen, C₁₋₄ alkyl, and C₃₋₆ cycloalkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —B(OR¹⁶)₂, —CN, —C(O)R¹⁶, —N(R¹⁶)C(O)R¹⁶, N(R¹⁶)S(O)₂R¹⁶, —N(R¹⁶)C(O)N(R¹⁶)₂, and 5-membered heterocycle; and R¹⁶ is selected from hydrogen, methyl, —CHF₂, morpholinyl, imidazolyl, and cyclopropyl, wherein the imidazolyl is optionally substituted with C₁₋₄ alkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —CN, —C(O)R¹⁶, —N(R¹⁶)C(O)R¹⁶, N(R¹⁶)S(O)₂R¹⁶, and 5-membered heterocycle; and R¹⁶ is selected from hydrogen, methyl, —CHF₂, morpholinyl, imidazolyl, and cyclopropyl, wherein the imidazolyl is optionally substituted with C₁₋₄ alkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —CN, and 5-membered heterocycle; and R¹⁶ is selected from hydrogen, methyl, —CHF₂, morpholinyl, imidazolyl, and cyclopropyl, wherein the imidazolyl is optionally substituted with C₁₋₄ alkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —N(R¹⁶)₂, —CN, and 5-membered heterocycle; and R¹⁶ is selected from hydrogen, methyl, —CHF₂, morpholinyl, imidazolyl, and cyclopropyl, wherein the imidazolyl is optionally substituted with C₁₋₄ alkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: halogen, C₁₋₄ alkyl, C₁₋₄ haloalkyl, —OR¹⁶, —CN, and 5-membered heterocycle; and R¹⁶ is selected from hydrogen, methyl, —CHF₂, morpholinyl, imidazolyl, and cyclopropyl, wherein the imidazolyl is optionally substituted with C₁₋₄ alkyl. In some embodiments, is selected from pyridine, isoquinoline, indazole, thiazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: fluoro, C₁₋₄ alkyl, C₁₋₄ haloalkyl, —OR¹⁶, —CN, and 5-membered heterocycle; and R¹⁶ is selected from hydrogen, methyl, —CHF₂, morpholinyl, imidazolyl, and cyclopropyl, wherein the imidazolyl is optionally substituted with C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected pyridine, isoquinoline, indazole, and phenyl, any of which is substituted by —C(O)H or —C(O)D, wherein R⁵ is further optionally substituted by one or more substituents independently selected from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶,
   —NO₂, ═O, ═S, ═N(R¹⁶), and —CN; and
4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, and phenyl, any of which is substituted by —C(O)H or —C(O)D, wherein R⁵ is further optionally substituted by one or more substituents independently selected from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, ═O, and —CN; and
4- to 6-membered heterocycle optionally substituted with one or more substituents independently selected from halogen, C₁₋₄ alkyl, C₁₋₄ haloalkyl, —OR¹⁶, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, and phenyl, any of which is substituted by —C(O)H or —C(O)D, wherein R⁵ is further optionally substituted by one or more substituents independently selected from halogen, C₁₋₄ alkyl, C₁₋₄ haloalkyl, —OR¹⁶, —SR¹⁶,
—N(R¹⁶)₂, —B(OR¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and —CN. In some embodiments, R⁵ is selected from pyridine, isoquinoline, indazole, and phenyl, any of which is substituted by —C(O)H, wherein R⁵ is further optionally substituted by one or more substituents independently selected from halogen, C₁₋₄ alkyl, C₁₋₄ haloalkyl, —OR¹⁶, —SR¹⁶,
—N(R¹⁶)₂, —B(OR¹⁶)₂, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, and phenyl, any of which is substituted by —C(O)H, wherein R⁵ is further optionally substituted by one or more substituents independently selected from halogen, C₁₋₄ alkyl, —OR¹⁶, and —N(R¹⁶)C(O)R¹⁶. In some embodiments, R⁵ is selected from 3- to 10-membered heterocycle and phenyl, any of which is substituted by —C(O)H, wherein R⁵ is further optionally substituted by one or more substituents independently selected from halogen, methyl, —OH, and —N(R¹⁶)C(O)R¹⁶.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, and phenyl, any of which is substituted by —C(O)H, wherein R⁵ is further optionally substituted by one or more substituents independently selected from fluoro, chloro, —OH, methyl,

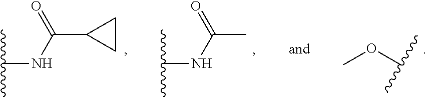

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, and —N(R¹⁶)C(O)N(R¹⁶)₂; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, and —N(R¹⁶)C(O)N(R¹⁶)₂; and R¹⁶ is selected from hydrogen, C₁₋₆ alkyl, C₃₋₆ cycloalkyl, 3- to 8-membered heterocycle, and —N(R²⁰)₂, wherein the 3- to 8-membered heterocycle is optionally substituted with methyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, and —N(R¹⁶)C(O)N(R¹⁶)₂; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with halogen.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₂₋₆ alkynyl, C₁₋₄ haloalkyl, —OR¹⁶, —N(R¹⁶)₂, —B(OR¹⁶)₂, —C(O)R¹⁶, —N(R¹⁶)C(O)R¹⁶, —N(R¹⁶)S(O)₂R¹⁶, —S(O)₂N(R¹⁶)₂, and —N(R¹⁶)C(O)N(R¹⁶)₂; and R¹⁶ is selected from hydrogen, C₁₋₆ alkyl, C₃₋₆ cycloalkyl, 3- to 8-membered heterocycle, and —N(R²⁰)₂, wherein the C₁₋₆ alkyl is optionally substituted with fluoro or chloro.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from 3- to 10-membered heteroaryl and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —B(OR¹⁶)₂, —C(O)R¹⁶, —N(R¹⁶)C(O)R¹⁶, —N(R¹⁶)S(O)₂R¹⁶, and —N(R¹⁶)C(O)N(R¹⁶)₂; and R¹⁶ is selected from hydrogen, methyl, —CHF₂, -morpholinyl, imidazolyl, and cyclopropyl, wherein the imidazolyl is optionally substituted with C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, thiazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —B(OR¹⁶)₂, C(O)R¹⁶, —N(R¹⁶)C(O)R¹⁶, —N(R¹⁶)S(O)₂R¹⁶, —S(O)₂N(R¹⁶)₂, and —N(R¹⁶)C(O)N(R¹⁶)₂; and R¹⁶ is selected from hydrogen, C₁₋₄ alkyl, and C₃₋₆ cycloalkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from pyridine, isoquinoline, indazole, thiazole, and phenyl, any of which is substituted by —C(O)H or —C(O)D; and wherein R⁵ is further optionally substituted by one or more substituents independently selected from: halogen, C₁₋₄ alkyl, C₃₋₆ alkynyl, C₁₋₄ haloalkyl, —OR¹⁶, —N(R¹⁶)₂, —B(OR¹⁶)₂, C(O)R¹⁶, —N(R¹⁶)C(O)R¹⁶, N(R¹⁶)S(O)₂R¹⁶, and —N(R¹⁶)C(O)N(R¹⁶)₂; and R¹⁶ is selected from hydrogen, methyl, —CHF₂, morpholinyl, imidazolyl, and cyclopropyl, wherein the imidazolyl is optionally substituted with C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected pyridine, isoquinoline, indazole, and phenyl, any of which is substituted by —C(O)H or —C(O)D; and wherein R⁵ is further optionally substituted by one or more substituents independently selected from: fluoro, chloro, methyl, —OH, —OCH₃, —OCHF₂, —CF₃, —B(OH)₂,

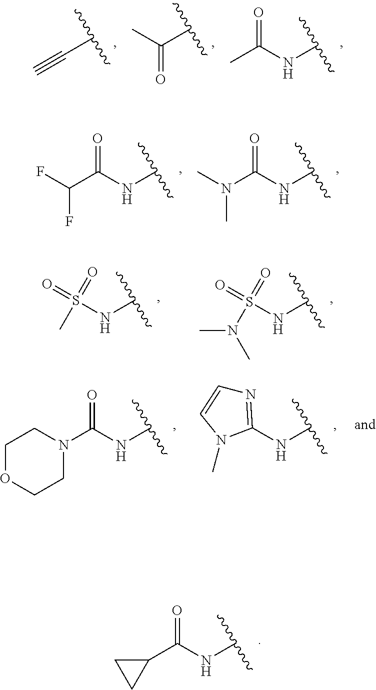

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected pyridine, isoquinoline, indazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: fluoro, chloro, methyl, —OH, —OCH₃, —OCHF₂, —CF₃, —B(OH)₂, —NH₂, —CN

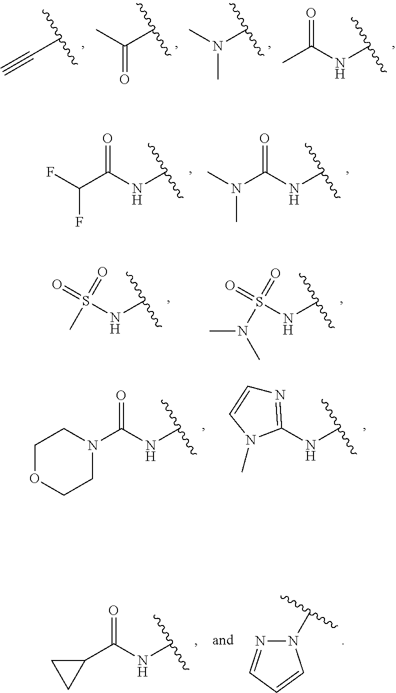

In some embodiments, R⁵ is selected pyridine, isoquinoline, indazole, benzothiazole, benzoxazole, pyrazolopyridine, benzimidazole, and phenyl, any of which is substituted by —C(O)H or —C(O)D; and wherein R⁵ is further optionally substituted by one or more substituents independently selected from: fluoro, chloro, methyl, —OH, —OCH₃, —OCHF₂, —CF₃, —CN,

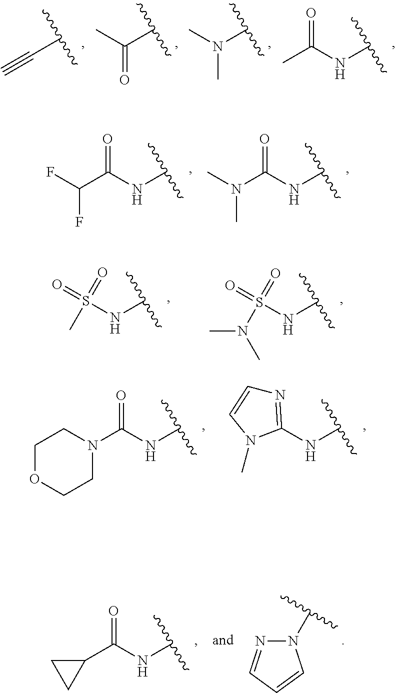

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from:

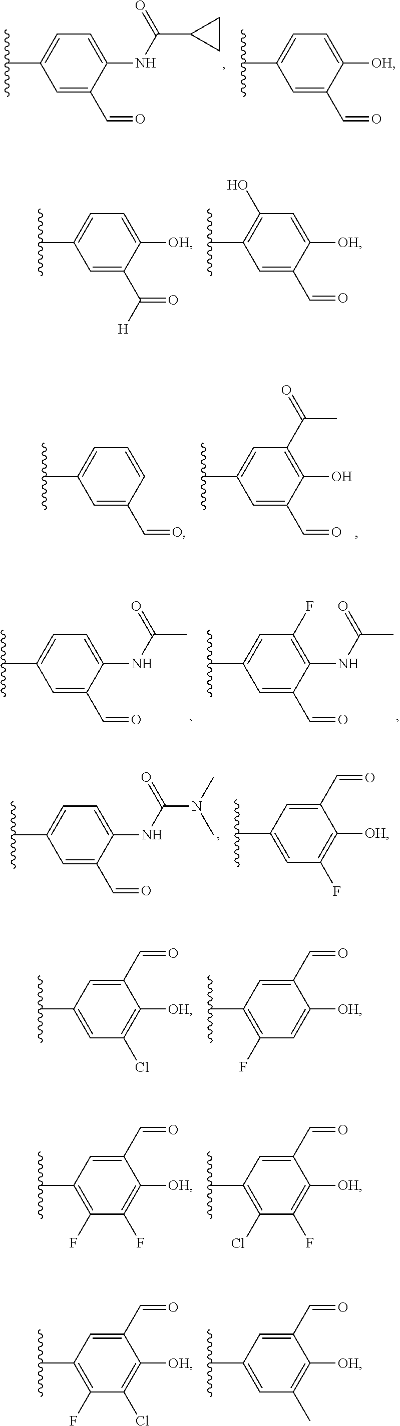

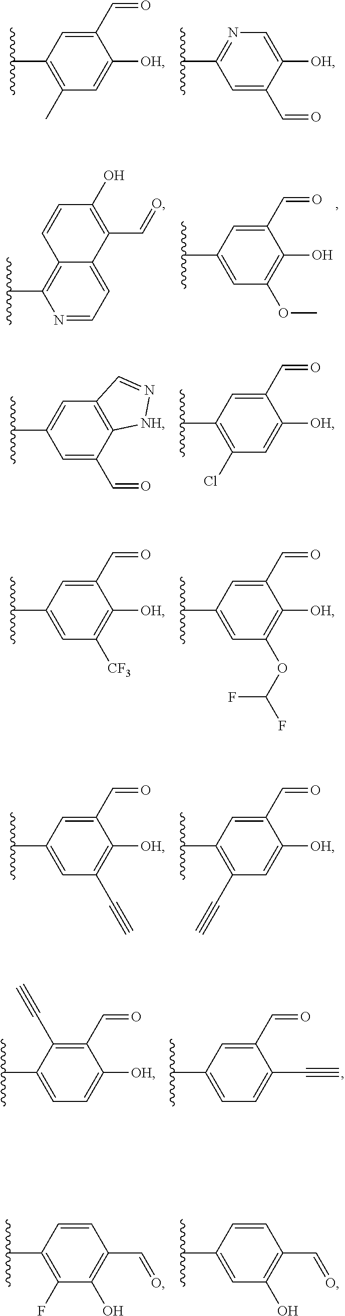

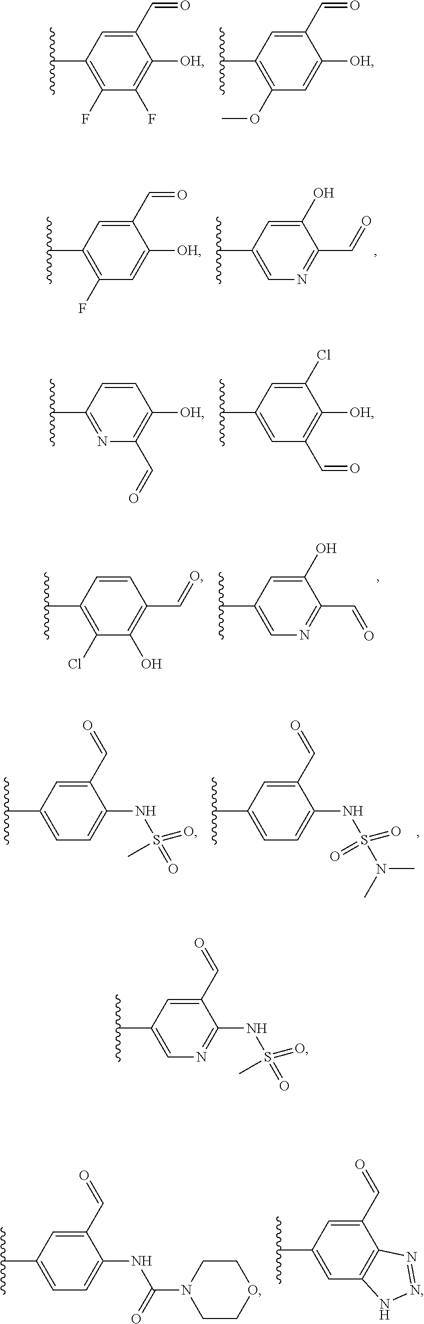

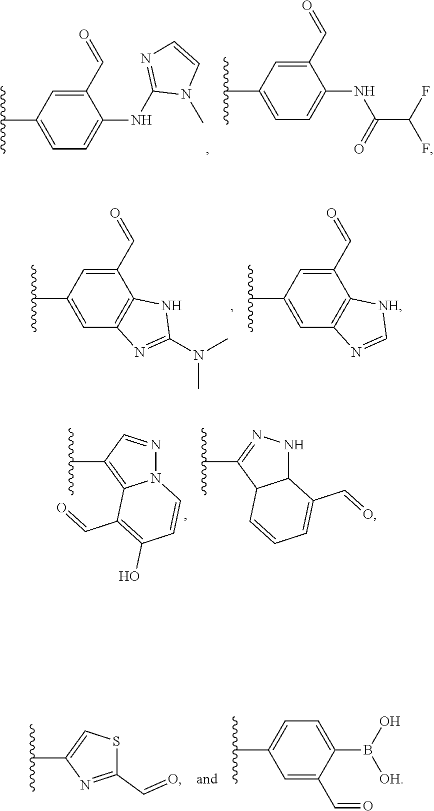

In some embodiments, for the compound or salt of Formula (A), (A-1), (I), (I-A), (II), B), (IX), (IX-A), or (IX-B), R⁵ is selected from:

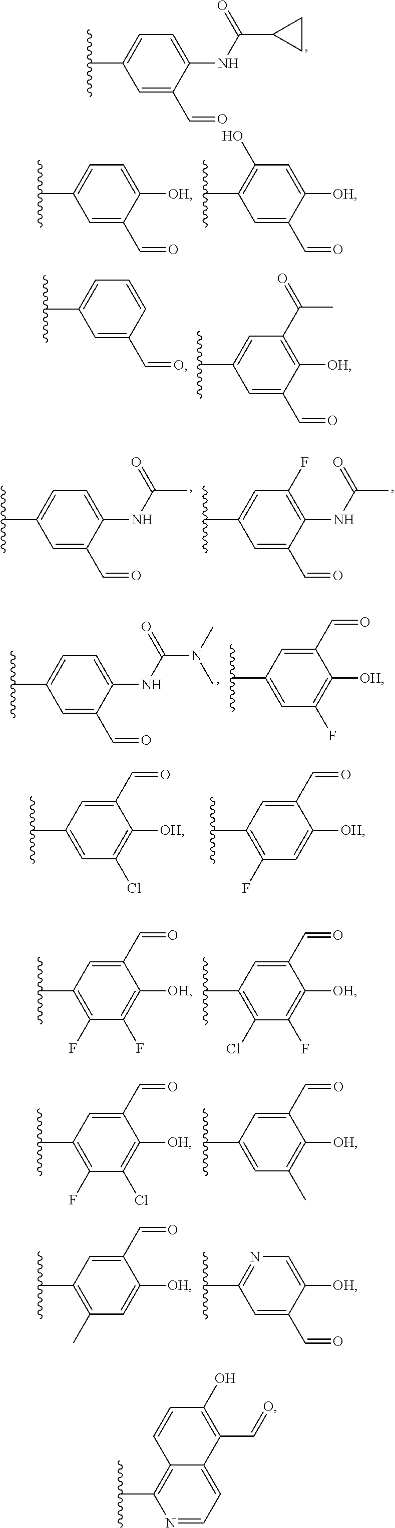

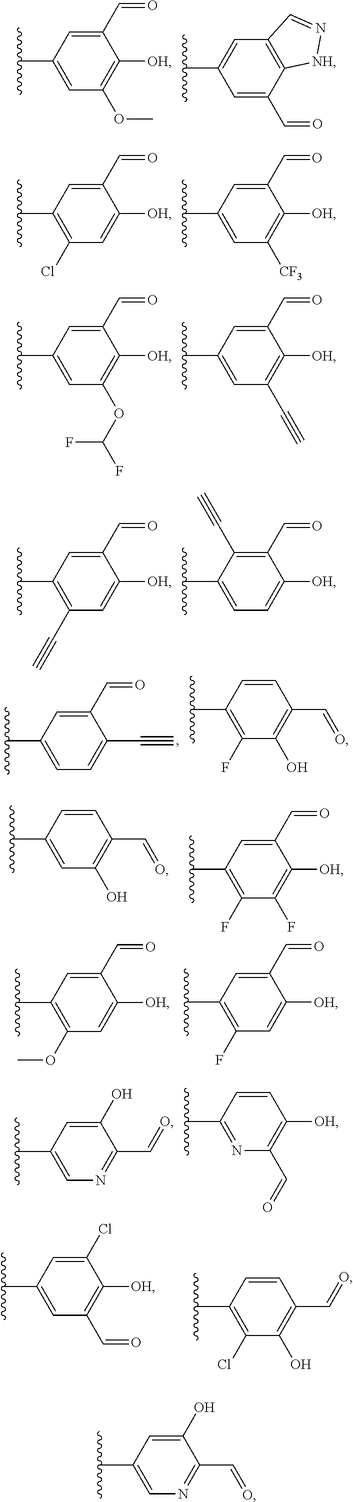

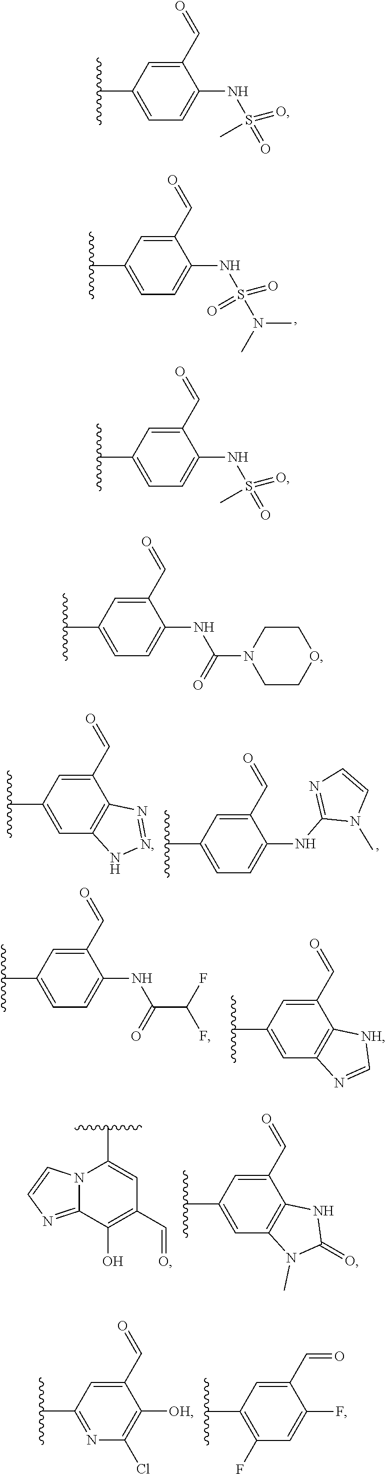

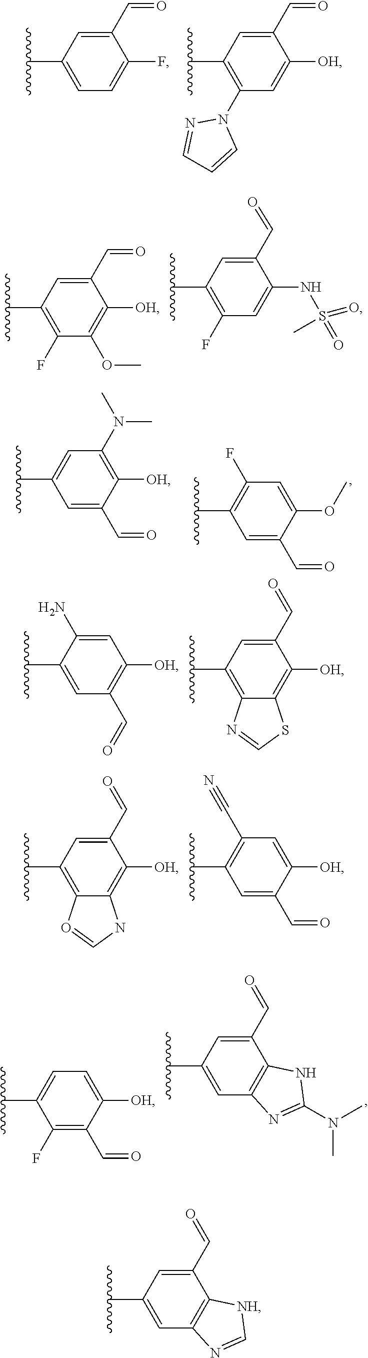

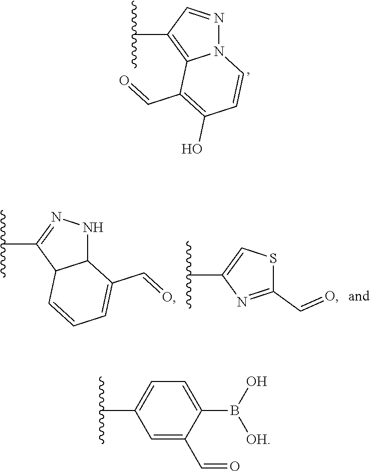

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (IX), (IX-A), or (IX-B), R⁵ is selected from:

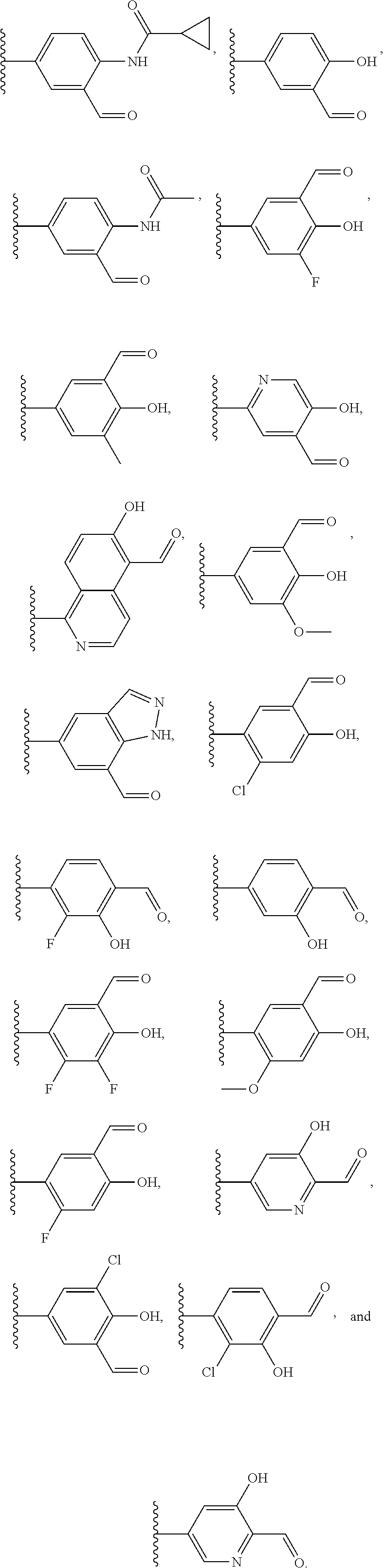

In some embodiments, the compound or salt of Formula (A), (A-1), (I), or (I-A) is represented by the structure of Formula (X):

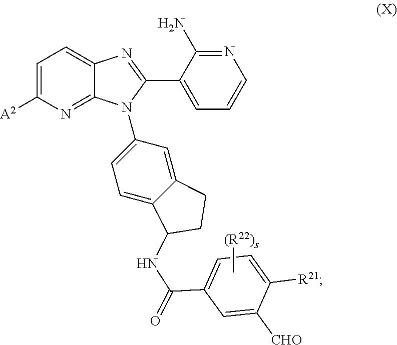

or a pharmaceutically acceptable salt thereof, wherein A² is defined as in Formula (A), (A-1), (I), or (I-A), and R²¹, R²², and s are each defined as in Formula (VII).

In some embodiments, the compound or salt of Formula (A), (A-1), (I), or (I-A) is represented by the structure of Formula (X-A):

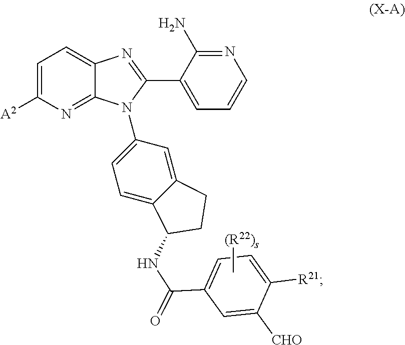

or a pharmaceutically acceptable salt thereof, wherein A² is defined as in Formula (A), (A-1), (I), or (I-A), and R²¹, R²², and s are each defined as in Formula (VII).

In some embodiments, the compound or salt of Formula (A), (A-1), (I), or (I-A) is represented by the structure of Formula (X-B):

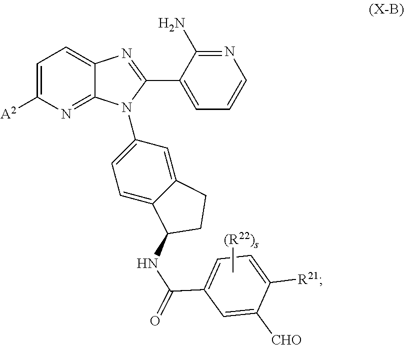

or a pharmaceutically acceptable salt thereof, wherein A² is defined as in Formula (A), (A-1), (I), or (I-A), and R²¹, R²², and s are each defined as in Formula (VII).

In some embodiments, for the compound or salt of Formula (VII), (VII-A), (VII-B), (X), (X-A), or (X-B), R²¹ is selected from:
hydrogen, halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN; and
4- to 6-membered heterocycle optionally substituted with one or more substituents independently selected from halogen, C₁₋₄ alkyl, C₁₋₄ haloalkyl, —OR¹⁶, and —CN.

In some embodiments, for the compound or salt Formula (VII), (VII-A), (VII-B), (X), (X-A), or (X-B), R²¹ is selected from hydrogen, halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —N(R¹⁶)C(O)N(R¹⁶)₂, and —CN; and wherein R¹⁶ is independently selected at each occurrence from hydrogen, C₁₋₆ carbocycle, and 3- to 6-membered heterocycle.

In some embodiments, for the compound or sale of Formula (VII), (VII-A), (VII-B), (X), (X-A), or (X-B), R²¹ is selected from hydrogen, halogen, C₁₋₄ alkyl, C₁₋₄ haloalkyl, —OR¹⁶, —N(R¹⁶)₂, —B(OR¹⁶)₂, and —CN; and wherein R¹⁶ is independently selected at each occurrence from hydrogen, C₁₋₆ carbocycle, and 3- to 6-membered heterocycle.

In some embodiments, for the compound or salt of Formula (VII), (VII-A), (VII-B), (X), (X-A), or (X-B), R²¹ is selected from hydrogen, C₁₋₄ haloalkyl, —OR¹⁶, —N(R¹⁶)₂, and —B(OR¹⁶)₂; and wherein R¹⁶ is independently selected at each occurrence from hydrogen, C₁₋₆ carbocycle, and 3- to 6-membered heterocycle.

In some embodiments, for the compound or salt of Formula (VII), (VII-A), (VII-B), (X), (X-A), or (X-B), R²¹ is selected from hydrogen, —OH, —CHF₂, —B(OH)₂, and —N(R¹⁶)₂. In some embodiments, R²¹ is selected from hydrogen, —OH, —CHF₂, —B(OH)₂, —NH₂,

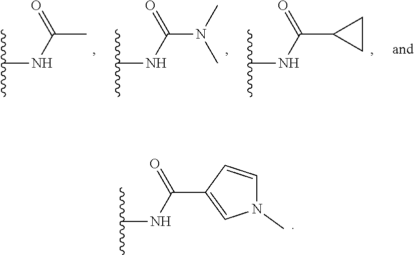

In some embodiments, R²¹ is selected from hydrogen and —OH. In some embodiments, R²¹ is —OH.

In some embodiments, the compound or salt of Formula (A), (A-1), (I), or (I-A) is represented by the structure of Formula (XI):

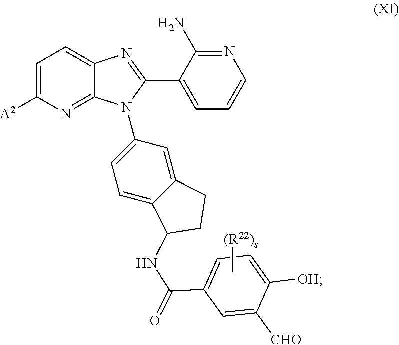

or a pharmaceutically acceptable salt thereof, wherein A² is defined as in Formula (A), (A-1), (I), or (I-A), and R²² and s are each defined as in Formula (VII).

In some embodiments, the compound or salt of Formula (A), (A-1), (I), or (I-A) is represented by the structure of Formula (XI-A):

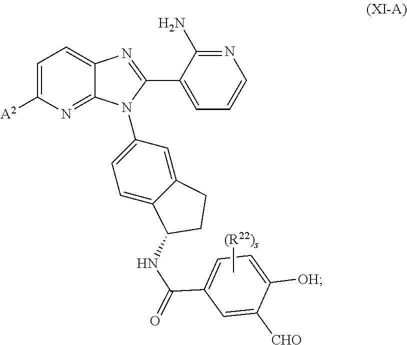

or a pharmaceutically acceptable salt thereof, wherein A² is defined as in Formula (A), (A-1), (I), or (I-A), and R²² and s are each defined as in Formula (VII).

In some embodiments, the compound or salt of Formula (A), (A-1), (I), or (I-A) is represented by the structure of Formula (XI-B):

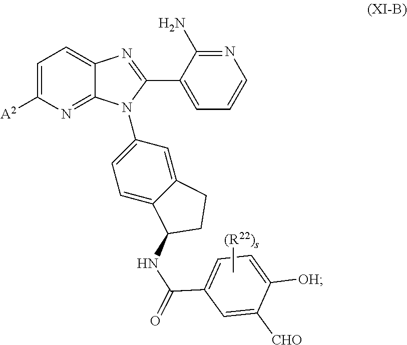

or a pharmaceutically acceptable salt thereof, wherein A² is defined as in Formula (A), (A-1), (I), or (I-A), and R²² and s are each defined as in Formula (VII).

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
5- to 10-membered heterocycle and C₃₋₁₀ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₃₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
      C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
hydrogen, halogen, C₁₋₄ haloalkyl, —OR¹¹, —N(R¹¹)₂, —C(O)N(R¹¹)₂, —N(R¹¹)C(O)R¹¹ and —CN;
C₁₋₆ alkyl, C₂₋₆ alkenyl, and C₂₋₆ alkynyl, any of which is optionally substituted with one or more substituents independently selected from:
   halogen, —OR¹¹, —N(R¹¹)₂, —C(O)N(R¹¹)₂, —N(R¹¹)C(O)R¹¹, ═O, and —CN; and
5- to 10-membered heterocycle and C₃₋₁₀ carbocycle, any of which is optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN;
   C₁₋₆ alkyl, C₂₋₆ alkenyl, and C₃₋₆ alkynyl, any one of which is optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, —NO₂, ═O, and —CN; and
C₃₋₁₀ carbocycle and 3- to 10-membered heterocycle; wherein the C₃₋₁₀ carbocycle and 3- to 10-membered heterocycle are each optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, ═O, and —CN; and
   C₁₋₆ alkyl, C₂₋₆ alkenyl, and C₃₋₆ alkynyl, any one of which is optionally substituted with one or more substituents independently selected from halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
hydrogen, halogen, C₁₋₄ haloalkyl, —OR¹¹, —N(R¹¹)₂, and —CN;
C₁₋₆ alkyl and C₂₋₆ alkynyl, any of which is optionally substituted with one or more halogen; and
5- to 10-membered heterocycle and C₃₋₁₀ carbocycle, any of which is optionally substituted with one or more substituents independently selected from:
   halogen, —OR¹¹, C₁₋₆ alkyl, C₁₋₆ haloalkyl, C₃₋₁₀ carbocycle and 3- to 10-membered heterocycle; wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
hydrogen, halogen, —OR¹¹, C₁₋₆ alkyl and C₂₋₆ alkynyl; and
5- to 10-membered heterocycle and C₃₋₁₀ carbocycle, any of which is optionally substituted with one or more substituents independently selected from:
   halogen, C₁₋₆ alkyl, C₁₋₆ haloalkyl, and 3- to 10-membered heterocycle; wherein the 3- to 10-membered heterocycle is optionally substituted with one or more C₁₋₆ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
hydrogen, halogen, C₁₋₄ haloalkyl, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and
C₁₋₆ alkyl, C₂₋₆ alkenyl, and C₂₋₆ alkynyl, any of which is optionally substituted with one or more substituents independently selected from:
   halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
hydrogen, halogen, C₁₋₄ haloalkyl, —OR¹¹, —N(R¹¹)₂, —C(O)N(R¹¹)₂, —N(R¹¹)C(O)R¹¹, and —CN; and
C₁₋₆ alkyl, C₂₋₆ alkenyl, and C₂₋₆ alkynyl, any of which is optionally substituted with one or more substituents independently selected from:
   halogen, —OR¹¹, —N(R¹¹)₂, —C(O)N(R¹¹)₂, —N(R¹¹)C(O)R¹¹, ═O, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from hydrogen, halogen, C₁₋₄ haloalkyl, —OR¹¹, —N(R¹¹)₂, and —CN, C₁₋₆ alkyl and C₂₋₆ alkynyl, any of which is optionally substituted with one or more halogen. In some embodiments, A² is selected from hydrogen, halogen, —OR¹¹, C₁₋₆ alkyl and C₂₋₆ alkynyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from: hydrogen, halogen, —OR¹¹, C₁₋₆ alkyl, and C₂₋₄ alkynyl; and R¹¹ is selected from hydrogen, C₁₋₄ alkyl, C₁₋₄ haloalkyl, and C₃₋₆ carbocycle.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from hydrogen, halogen, C₁₋₆ alkyl, C₂₋₄ alkynyl, —OH, —OCl₄ alkyl, and —OC₃₋₆ carbocycle. In some embodiments, A² is selected from hydrogen, fluoro, bromo, methyl, ethyl, ethynylene, trifluoromethyl, —OCH₃, and

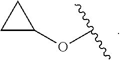 [SMILES: *OC1CC1]

In some embodiments, A² is selected from hydrogen, bromo, methyl, ethyl, —OCH₃, and

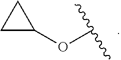 [SMILES: *OC1CC1]

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
5- to 10-membered heterocycle and C₃₋₁₀ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N (R¹¹)C(O)N(R¹¹)₂, —S(O)R¹¹, —S(O)₂R¹¹, —S(O)₂N(R¹¹)₂, —N₂, ═O, ═S, ═N(R¹¹), and —CN;
   C₁₋₆ alkyl, C₂₋₆ alkenyl, and C₃₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
      C₁₋₆ alkyl, C₂₋₆ alkenyl, and C₃₋₆ alkynyl, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
5- to 10-membered heterocycle and C₃₋₁₀ carbocycle, any of which is optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN;
   C₁₋₆ alkyl, C₂₋₆ alkenyl, and C₃₋₆ alkynyl, any one of which is optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, —NO₂, ═O, and —CN; and
   C₃₋₁₀ carbocycle and 3- to 10-membered heterocycle; wherein the C₃₋₁₀ carbocycle and 3- to 10-membered heterocycle are each optionally substituted with one or more substituents independently selected from:
      halogen, —OR¹¹, —N(R¹¹)₂, —C(O)R¹¹, —C(O)N(R¹¹)₂, —N(R¹¹)C(O)R¹¹, ═O, and —CN; and
      C₁₋₆ alkyl, C₂₋₆ alkenyl, and C₃₋₆ alkynyl, any one of which is optionally substituted with one or more substituents independently selected from:
         halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from:
5- to 10-membered heterocycle and C₃₋₁₀ carbocycle, any of which is optionally substituted with one or more substituents independently selected from:
   halogen, —OR¹¹, C₁₋₆ alkyl, C₁₋₆ haloalkyl, C₃₋₁₀ carbocycle and 3- to 10-membered heterocycle; wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from 5- to 10-membered heterocycle and C₃₋₁₀ carbocycle, any of which is optionally substituted with one or more substituents independently selected from halogen, C₁₋₆ alkyl, C₁₋₆ haloalkyl, and 3- to 10-membered heterocycle; wherein the 3- to 10-membered heterocycle is optionally substituted with one or more C₁₋₆ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from 5- to 10-membered heterocycle and C₃₋₁₀ carbocycle, any of which is optionally substituted with one or more substituents independently selected from fluoro, methyl, and N-methylpiperazinyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from phenyl, morpholinyl, piperazinyl, pyrazolyl, pyridinyl, pyridazinyl, and pyrazinyl, any of which is optionally substituted with one or more substituents independently selected from halogen, —OR¹¹ C₁₋₆ alkyl, C₁₋₆ haloalkyl, C₃₋₁₀ carbocycle, and 3- to 6-membered heterocycle; wherein the C₃₋₁₀ carbocycle and 3- to 10-membered heterocycle are each optionally substituted with one or more C₁₋₆ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from phenyl, morpholinyl, piperazinyl, pyrazolyl, pyridinyl, pyridazinyl, and pyrazinyl, any of which is optionally substituted with one or more substituents independently selected from halogen, C₁₋₆ alkyl, C₁₋₆ haloalkyl, and 3- to 6-membered heterocycle; wherein the 3- to 10-membered heterocycle is optionally substituted with one or more C₁₋₆ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from phenyl, morpholinyl, piperazinyl, pyrazolyl, pyridinyl, pyridazinyl, and pyrazinyl, any of which is optionally substituted with one or more substituents independently selected from fluoro, methyl, and N-methylpiperazinyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), A² is selected from cyclopropyl, phenyl,

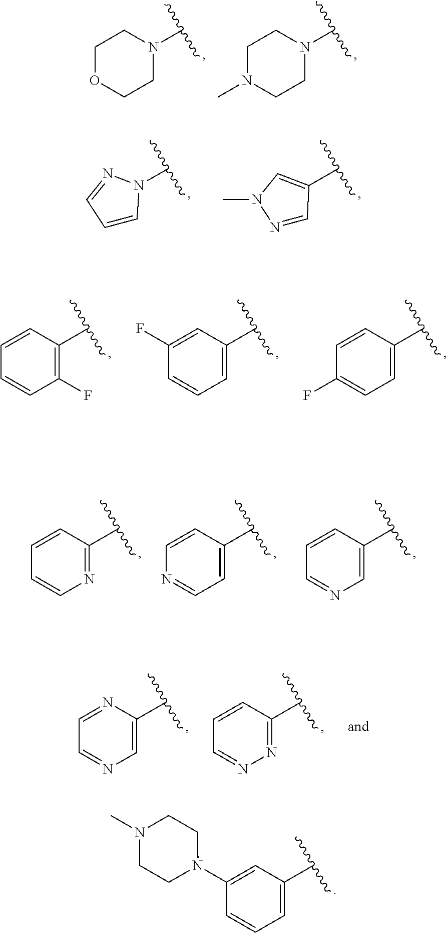

In some embodiments, for the compound or salt of Formula (VII), (VII-A), (VII-B), (VIII), (VIII-A), (VIII-B), (X), (X-A), (X-B), (XI), (XI-A), or (XI-B), R²² is independently selected at each instance from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, and —CN; and
4- to 6-membered heterocycle optionally substituted with one or more substituents independently selected from: halogen, C₁₋₄ alkyl, C₁₋₄ haloalkyl, —OR¹⁶, —SR¹⁶, —N(R¹⁶)₂, —NO₂, and —CN.

In some embodiments, for the compound or salt of Formula (VII), (VII-A), (VII-B), (VIII), (VIII-A), (VIII-B), (X), (X-A), (X-B), (XI), (XI-A), or (XI-B), R²² is independently selected at each instance from:
halogen, C₁₋₄ alkyl, C₁₋₄ haloalkyl, —OR¹⁶, —SR¹⁶, —N(R¹⁶)₂, —B(OR¹⁶)₂, —C(O)R¹⁶, —C(O)N(R¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN; and
4- to 6-membered heterocycle optionally substituted with one or more substituents independently selected from halogen, C₁₋₄ alkyl, C₁₋₄ haloalkyl, —OR¹⁶, and —CN.

In some embodiments, for the compound or salt of Formula (VII), (VII-A), (VII-B), (VIII), (VIII-A), (VIII-B), (X), (X-A), (X-B), (XI), (XI-A), or (XI-B), R²² is independently selected at each instance from halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN. In some embodiments, R²² is independently selected at each instance from halogen, C₁₋₄ alkyl, C₁₋₄ haloalkyl, —OR¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, and —CN.

In some embodiments, for the compound or salt of Formula (VII), (VII-A), (VII-B), (VIII), (VIII-A), (VIII-B), (X), (X-A), (X-B), (XI), (XI-A), or (XI-B), R²² is independently selected at each instance from halogen, C₁₋₄ alkyl, —OR¹⁶, and —N(R¹⁶)C(O)R¹⁶. In some embodiments, R²² is independently selected at each instance from halogen, methyl, —OH, and —N(R¹⁶)C(O)R¹⁶.

In some embodiments, for the compound or salt of Formula (VII), (VII-A), (VII-B), (VIII), (VIII-A), (VIII-B), (X), (X-A), (X-B), (XI), (XI-A), or (XI-B), R²² is independently selected at each instance from fluoro, chloro, —OH, methyl,

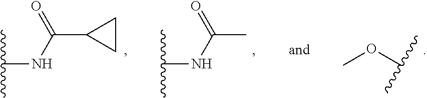

In some embodiments, for the compound or salt of Formula (VII), (VII-A), (VII-B), (VIII), (VIII-A), (VIII-B), (X), (X-A), (X-B), (XI), (XI-A), or (XI-B), s is selected from 0, 1, 2, and 3. In some embodiments, s is selected from 0, 1 and 2. In some embodiments, s is selected from 0 and 1. In some embodiments, s is 0.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹⁶ is independently selected at each occurrence from:
hydrogen;
C₁₋₄ alkyl, wherein the C₁₋₄ alkyl is optionally substituted with one or more substituents independently selected from:
   halogen, —OR²⁰, —SR²⁰, —N(R²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN;
   C₃₋₈ carbocycle and 3- to 8-membered heterocycle, either of which is optionally substituted with one or more substituents independently selected from: halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
C₃₋₈ carbocycle and 3- to 8-membered heterocycle, either of which is optionally substituted with one or more substituents independently selected from halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —NO₂, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹⁶ is independently selected at each occurrence from:
hydrogen;
C₁₋₄ alkyl, wherein the C₁₋₄ alkyl is optionally substituted with one or more substituents independently selected from:
   halogen, —OR²⁰, —N(R²⁰)₂, —C(O)R²⁰, —C(O)N(R²⁰)₂, —N(R²⁰)C(O)R²⁰, and —CN; and
   C₃₋₈ carbocycle and 3- to 8-membered heterocycle, either of which is optionally substituted with one or more substituents independently selected from: halogen, C₁₋₄ alkyl, C₁₋₄ haloalkyl, —OR²⁰, —N(R²⁰)₂, —B(OR²⁰)₂, —C(O)R²⁰, —C(O)N(R²⁰)₂, —N(R²⁰)C(O)R²⁰, and —CN; and
C₃₋₈ carbocycle and 3- to 8-membered heterocycle, either of which is optionally substituted with one or more substituents independently selected from halogen, C₁₋₄ alkyl, C₁₋₄ haloalkyl, —OR²⁰, —N(R²⁰)₂, —C(O)R²⁰, —C(O)N(R²⁰)₂, —N(R²⁰)C(O)R²⁰, and —CN.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹⁶ is independently selected at each occurrence from:
hydrogen, C₁₋₄ alkyl, C₁₋₄ haloalkyl and C₃₋₈ carbocycle optionally substituted with one or more —B(OR²⁰)₂; and
C₃₋₈ carbocycle and 3- to 8-membered heterocycle, either of which is optionally substituted with one or more substituents independently selected from halogen, C₁₋₄ alkyl, and, —OR²⁰.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹⁶ is independently selected at each occurrence from:
hydrogen, C₁₋₄ alkyl, C₁₋₄ haloalkyl and C₃₋₈ carbocycle optionally substituted with one or more —B(OR²⁰)₂; and
C₃₋₈ carbocycle and 3- to 8-membered heterocycle, either of which is optionally substituted with one or more substituents independently selected from halogen, C₁₋₄ alkyl, and, —OR²⁰; and
R²⁰ is independently selected from hydrogen and C₁₋₄ alkyl.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹⁶ is independently selected at each occurrence from hydrogen, C₁₋₄ alkyl, and C₃₋₈ carbocycle. In some embodiments, R¹⁶ is independently selected at each occurrence from hydrogen, C₁₋₄ alkyl, and C₃₋₆ carbocycle. In some embodiments, R¹⁶ is independently selected at each occurrence from hydrogen, methyl, and cyclopropyl. In some embodiments, R¹⁶ is selected at each occurrence from hydrogen and methyl. In some embodiments, R¹⁶ is hydrogen.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R²⁰ is independently selected at each occurrence from hydrogen, C₁₋₄ alkyl, C₁₋₄ haloalkyl, C₃₋₈ carbocycle, and 3- to 8-membered heterocycle. In some embodiments, R²⁰ is independently selected at each occurrence from hydrogen and C₁₋₄ alkyl. In some embodiments, R²⁰ is hydrogen.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹⁰, R, R¹², R¹³, R¹⁴, and R¹⁵ are each independently selected at each occurrence from hydrogen, C₁₋₄ alkyl, C₃₋₈ carbocycle, 3- to 8-membered heterocycle, and C₁₋₄ haloalkyl. In some embodiments, R¹⁰, R, R¹², R¹³, R¹⁴, and R¹⁵ are each independently selected at each occurrence from hydrogen, C₁₋₄ alkyl, C₃₋₆ carbocycle, 3- to 6-membered heterocycle, and C₁₋₄ haloalkyl. In some embodiments, R¹⁰, R, R¹², R¹³, R¹⁴, and R¹⁵ are each independently selected at each occurrence from hydrogen and C₁₋₄ alkyl. In some embodiments, R¹⁰, R, R¹², R¹³, R¹⁴, and R¹⁵ are each hydrogen.

In some embodiments, for the compound or salt of Formula (A), (A-1), (I), (I-A), (II), (II-A), or (II-B), R¹⁰ is independently selected at each occurrence from hydrogen, C₁₋₄ alkyl, C₃₋₈ carbocycle, 3- to 8-membered heterocycle, and C₁₋₄ haloalkyl. In some embodiments, R¹⁰ is independently selected at each occurrence from hydrogen and C₁₋₄ alkyl. In some embodiments, R¹⁰ is hydrogen.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), R¹¹ is independently selected at each occurrence from: hydrogen, C₁₋₄ alkyl, C₃₋₈ carbocycle, 3- to 8-membered heterocycle, and C₁₋₄ haloalkyl. In some embodiments, R¹¹ is independently selected at each occurrence from: hydrogen, C₁₋₄ alkyl, and C₃₋₆ carbocycle. In some embodiments, R¹¹ is independently selected at each occurrence from hydrogen and C₁₋₄ alkyl. In some embodiments, R¹¹ is hydrogen.

In some embodiments, for the compound or salt of Formula (A), (A-1), (I), (I-A), (II), (II-A), or (II-B), R¹² is independently selected at each occurrence from: hydrogen, C₁₋₄ alkyl, C₃₋₈ carbocycle, 3- to 8-membered heterocycle, and C₁₋₄ haloalkyl. In some embodiments, R¹² is independently selected at each occurrence from hydrogen and C₁₋₄ alkyl. In some embodiments, R¹² is hydrogen.

In some embodiments, for the compound or salt of Formula (A), (A-1), (I), (I-A), (II), (II-A), (II-B), (III), (III-A), (III-B), (IV), (IV-A), or (IV-B), R¹³ is independently selected at each occurrence from: hydrogen, C₁₋₄ alkyl, C₃₋₈ carbocycle, 3- to 8-membered heterocycle, and C₁₋₄ haloalkyl. In some embodiments, R¹³ is independently selected at each occurrence from hydrogen and C₁₋₄ alkyl. In some embodiments, R¹³ is hydrogen.

In some embodiments, for the compound or salt of Formula (A), (A-1), (I), (I-A), (II), (II-A), (II-B), (III), (III-A), (III-B), (IV), (IV-A), or (IV-B), R¹⁴ is independently selected at each occurrence from: hydrogen, C₁₋₄ alkyl, C₃₋₈ carbocycle, 3- to 8-membered heterocycle, and C₁₋₄ haloalkyl. In some embodiments, R¹⁴ is independently selected at each occurrence from hydrogen and C₁₋₄ alkyl. In some embodiments, R¹⁴ is hydrogen.

In some embodiments, for the compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), or (VIII-B), R¹⁵ is independently selected at each occurrence from: hydrogen, C₁₋₄ alkyl, C₃₋₈ carbocycle, 3- to 8-membered heterocycle, and C₁₋₄ haloalkyl. In some embodiments, R¹⁵ is independently selected at each occurrence from hydrogen and C₁₋₄ alkyl. In some embodiments, R¹⁵ is independently selected at each occurrence from hydrogen and methyl. In some embodiments, R¹⁵ is hydrogen.

In some embodiments, the compound or salt of Formula (A), (A-1), (I), or (I-A) is a compound of Table 1. In some embodiments, the compounds or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), is a compound of Table 1.

TABLE 1
Chemical structures of selected compounds.

| Cmpd. | |
|---|---|
| No. | Structure |
| 1 | 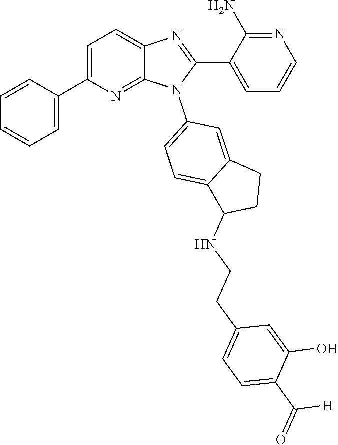 [SMILES: Nc1ncccc1-c1nc2ccc(-c3ccccc3)nc2n1-c1ccc2c(c1)CCC2NCCc1ccc(C=O)c(O)c1] |
| 2 | 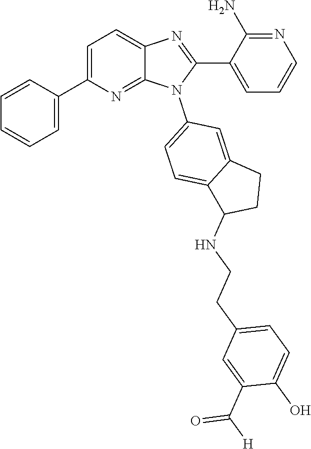 [SMILES: Nc1ncccc1-c1nc2ccc(-c3ccccc3)nc2n1-c1ccc2c(c1)CCC2NCCc1ccc(O)c(C=O)c1] |
| 3 | 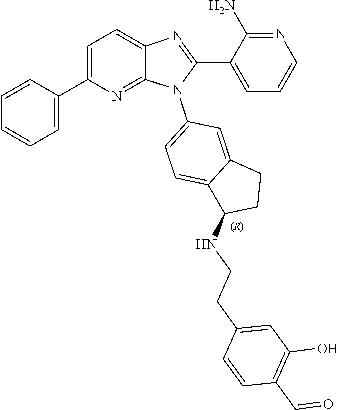 |
| 4 | 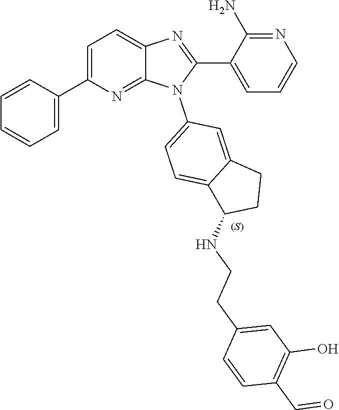 |
| 5 | 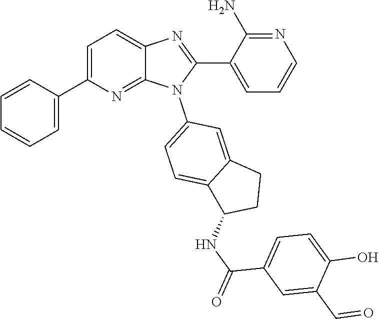 [SMILES: Nc1ncccc1-c1nc2ccc(-c3ccccc3)nc2n1-c1ccc2c(c1)CC[C@@H]2NC(=O)c1ccc(O)c(C=O)c1] |
| 6 | 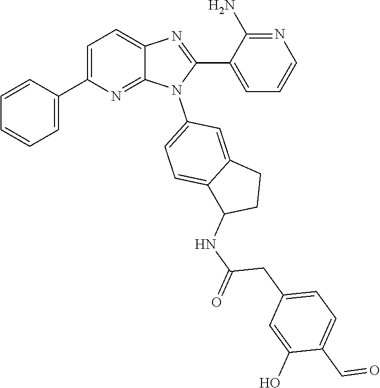 [SMILES: Nc1ncccc1-c1nc2ccc(-c3ccccc3)nc2n1-c1ccc2c(c1)CCC2NC(=O)Cc1ccc(C=O)c(O)c1] |
| 7 | 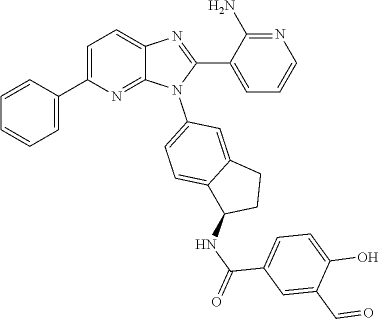 [SMILES: Nc1ncccc1-c1nc2ccc(-c3ccccc3)nc2n1-c1ccc2c(c1)CC[C@H]2NC(=O)c1ccc(O)c(C=O)c1] |
| 8 | 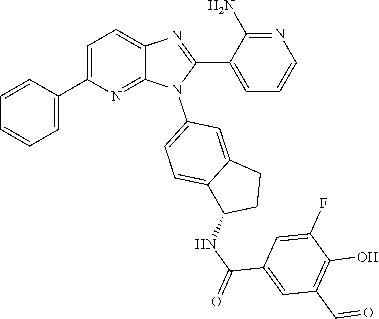 [SMILES: Nc1ncccc1-c1nc2ccc(-c3ccccc3)nc2n1-c1ccc2c(c1)CC[C@@H]2NC(=O)c1cc(F)c(O)c(C=O)c1] |
| 9 | 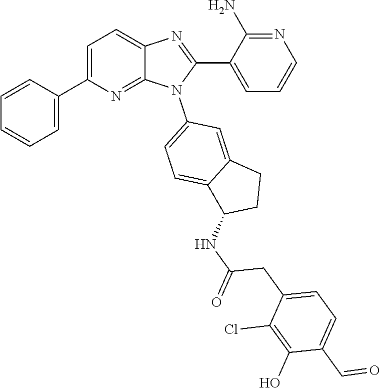 [SMILES: Nc1ncccc1-c1nc2ccc(-c3ccccc3)nc2n1-c1ccc2c(c1)CC[C@@H]2NC(=O)Cc1ccc(C=O)c(O)c1Cl] |
| 10 | 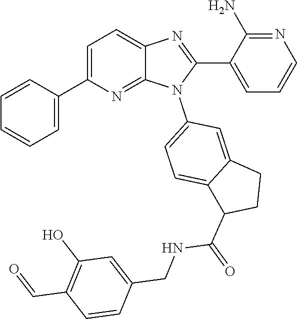 [SMILES: Nc1ncccc1-c1nc2ccc(-c3ccccc3)nc2n1-c1ccc2c(c1)CCC2C(=O)NCc1ccc(C=O)c(O)c1] |
| 11 | 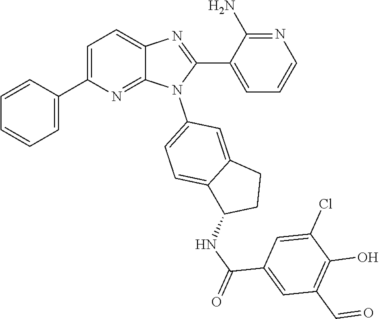 [SMILES: Nc1ncccc1-c1nc2ccc(-c3ccccc3)nc2n1-c1ccc2c(c1)CC[C@@H]2NC(=O)c1cc(Cl)c(O)c(C=O)c1] |
| 12 | 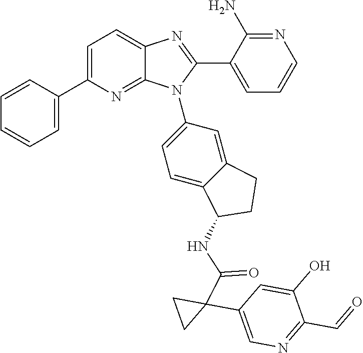 [SMILES: Nc1ncccc1-c1nc2ccc(-c3ccccc3)nc2n1-c1ccc2c(c1)CC[C@@H]2NC(=O)C1(c2cnc(C=O)c(O)c2)CC1] |
| 13 | 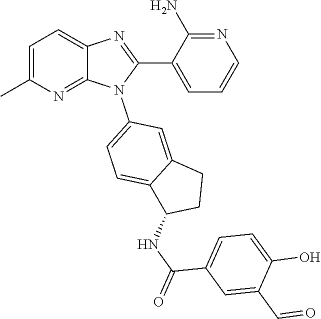 [SMILES: Cc1ccc2nc(-c3cccnc3N)n(-c3ccc4c(c3)CC[C@@H]4NC(=O)c3ccc(O)c(C=O)c3)c2n1] |
| 14 | 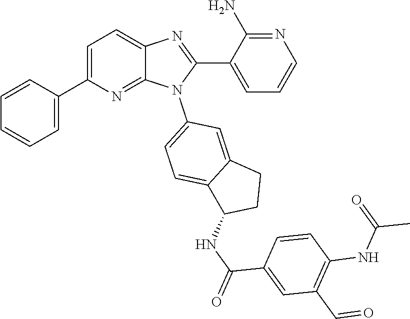 [SMILES: CC(=O)Nc1ccc(C(=O)N[C@H]2CCc3cc(-n4c(-c5cccnc5N)nc5ccc(-c6ccccc6)nc54)ccc32)cc1C=O] |
| 15 | 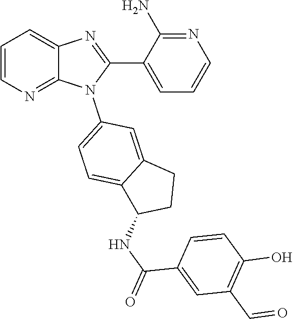 [SMILES: Nc1ncccc1-c1nc2cccnc2n1-c1ccc2c(c1)CC[C@@H]2NC(=O)c1ccc(O)c(C=O)c1] |
| 16 | 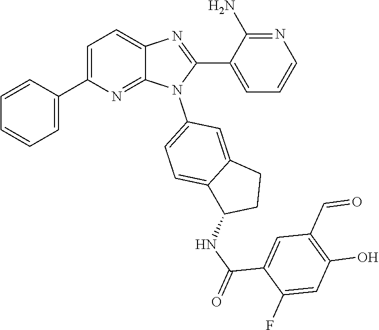 [SMILES: Nc1ncccc1-c1nc2ccc(-c3ccccc3)nc2n1-c1ccc2c(c1)CC[C@@H]2NC(=O)c1cc(C=O)c(O)cc1F] |
| 17 | 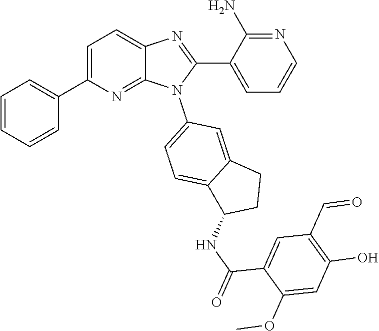 [SMILES: COc1cc(O)c(C=O)cc1C(=O)N[C@H]1CCc2cc(-n3c(-c4cccnc4N)nc4ccc(-c5ccccc5)nc43)ccc21] |
| 18 | 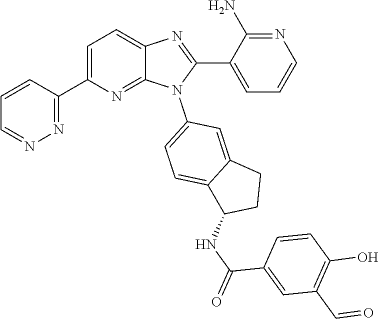 [SMILES: Nc1ncccc1-c1nc2ccc(-c3cccnn3)nc2n1-c1ccc2c(c1)CC[C@@H]2NC(=O)c1ccc(O)c(C=O)c1] |
| 19 | 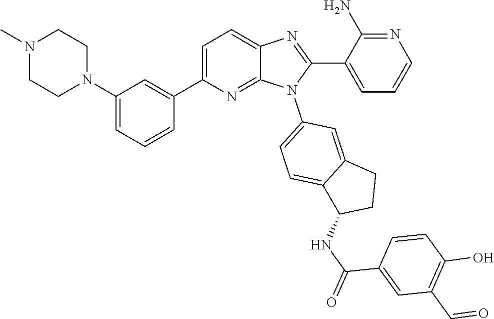 [SMILES: CN1CCN(c2cccc(-c3ccc4nc(-c5cccnc5N)n(-c5ccc6c(c5)CC[C@@H]6NC(=O)c5ccc(O)c(C=O)c5)c4n3)c2)CC1] |
| 20 | 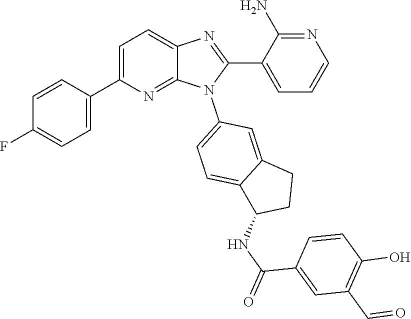 [SMILES: Nc1ncccc1-c1nc2ccc(-c3ccc(F)cc3)nc2n1-c1ccc2c(c1)CC[C@@H]2NC(=O)c1ccc(O)c(C=O)c1] |
| 21 | 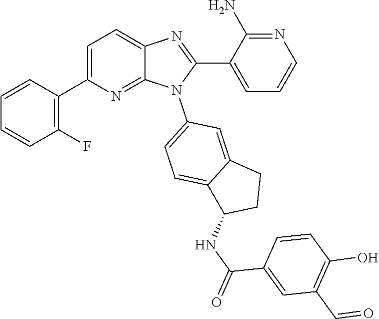 [SMILES: Nc1ncccc1-c1nc2ccc(-c3ccccc3F)nc2n1-c1ccc2c(c1)CC[C@@H]2NC(=O)c1ccc(O)c(C=O)c1] |
| 22 | 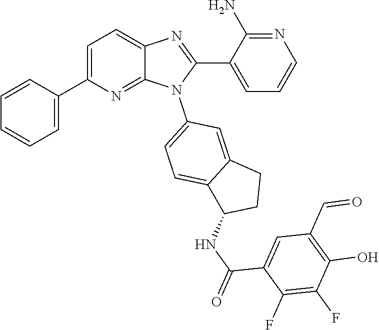 [SMILES: Nc1ncccc1-c1nc2ccc(-c3ccccc3)nc2n1-c1ccc2c(c1)CC[C@@H]2NC(=O)c1cc(C=O)c(O)c(F)c1F] |
| 23 | 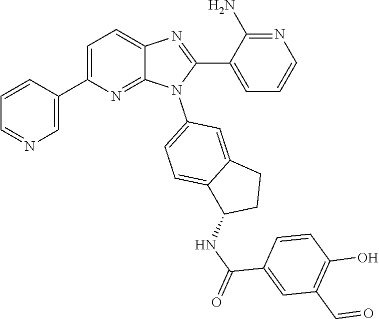 [SMILES: Nc1ncccc1-c1nc2ccc(-c3cccnc3)nc2n1-c1ccc2c(c1)CC[C@@H]2NC(=O)c1ccc(O)c(C=O)c1] |
| 24 | 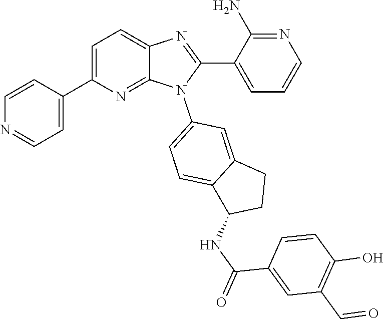 [SMILES: Nc1ncccc1-c1nc2ccc(-c3ccncc3)nc2n1-c1ccc2c(c1)CC[C@@H]2NC(=O)c1ccc(O)c(C=O)c1] |
| 25 | 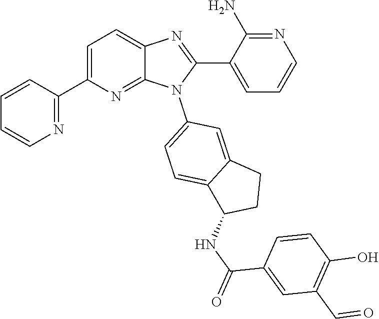 [SMILES: Nc1ncccc1-c1nc2ccc(-c3ccccn3)nc2n1-c1ccc2c(c1)CC[C@@H]2NC(=O)c1ccc(O)c(C=O)c1] |
| 26 | 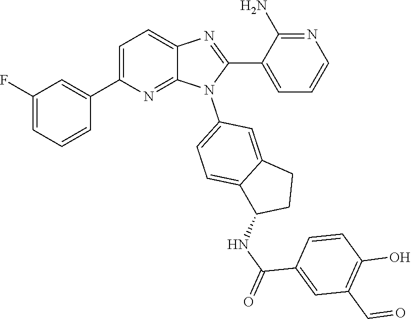 [SMILES: Nc1ncccc1-c1nc2ccc(-c3cccc(F)c3)nc2n1-c1ccc2c(c1)CC[C@@H]2NC(=O)c1ccc(O)c(C=O)c1] |
| 27 | 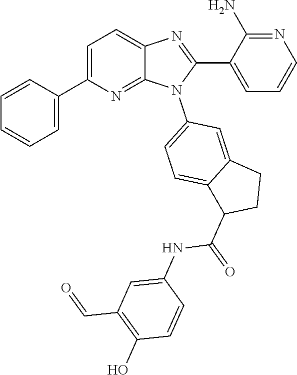 [SMILES: Nc1ncccc1-c1nc2ccc(-c3ccccc3)nc2n1-c1ccc2c(c1)CCC2C(=O)Nc1ccc(O)c(C=O)c1] |
| 28 | 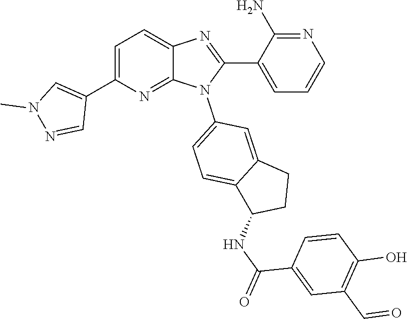 [SMILES: Cn1cc(-c2ccc3nc(-c4cccnc4N)n(-c4ccc5c(c4)CC[C@@H]5NC(=O)c4ccc(O)c(C=O)c4)c3n2)cn1] |
| 29 | 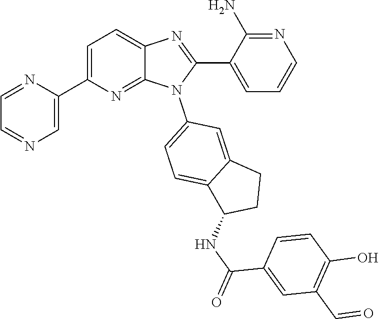 [SMILES: Nc1ncccc1-c1nc2ccc(-c3cnccn3)nc2n1-c1ccc2c(c1)CC[C@@H]2NC(=O)c1ccc(O)c(C=O)c1] |
| 30 | 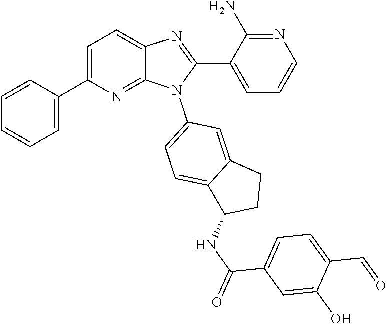 [SMILES: Nc1ncccc1-c1nc2ccc(-c3ccccc3)nc2n1-c1ccc2c(c1)CC[C@@H]2NC(=O)c1ccc(C=O)c(O)c1] |
| 31 | 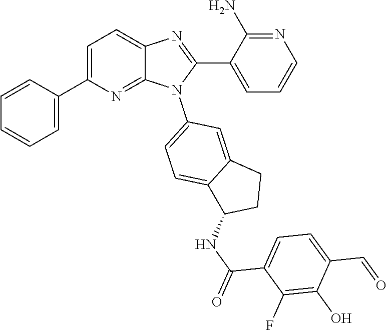 [SMILES: Nc1ncccc1-c1nc2ccc(-c3ccccc3)nc2n1-c1ccc2c(c1)CC[C@@H]2NC(=O)c1ccc(C=O)c(O)c1F] |
| 32 | 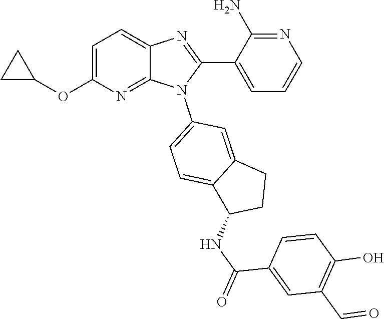 [SMILES: Nc1ncccc1-c1nc2ccc(OC3CC3)nc2n1-c1ccc2c(c1)CC[C@@H]2NC(=O)c1ccc(O)c(C=O)c1] |
| 33 | 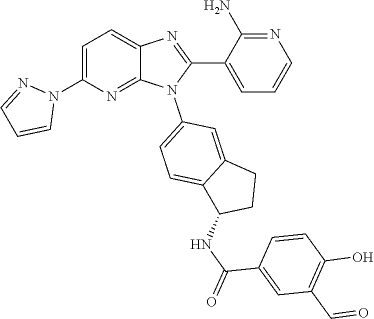 [SMILES: Nc1ncccc1-c1nc2ccc(-n3cccn3)nc2n1-c1ccc2c(c1)CC[C@@H]2NC(=O)c1ccc(O)c(C=O)c1] |
| 34 | 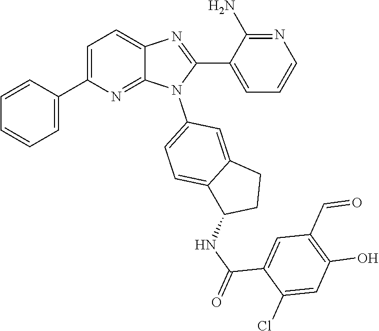 [SMILES: Nc1ncccc1-c1nc2ccc(-c3ccccc3)nc2n1-c1ccc2c(c1)CC[C@@H]2NC(=O)c1cc(C=O)c(O)cc1Cl] |
| 35 | 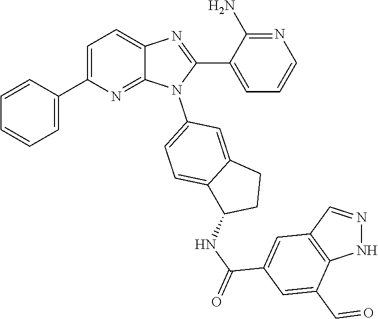 [SMILES: Nc1ncccc1-c1nc2ccc(-c3ccccc3)nc2n1-c1ccc2c(c1)CC[C@@H]2NC(=O)c1cc(C=O)c2[nH]ncc2c1] |
| 36 | 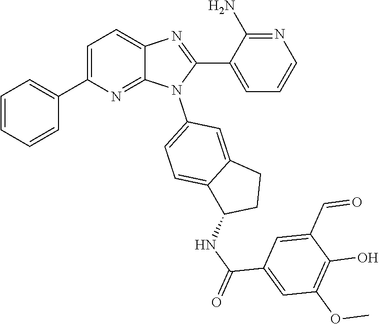 [SMILES: COc1cc(C(=O)N[C@H]2CCc3cc(-n4c(-c5cccnc5N)nc5ccc(-c6ccccc6)nc54)ccc32)cc(C=O)c1O] |
| 37 | 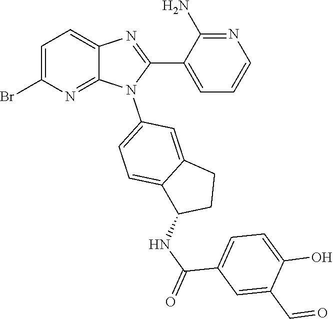 [SMILES: Nc1ncccc1-c1nc2ccc(Br)nc2n1-c1ccc2c(c1)CC[C@@H]2NC(=O)c1ccc(O)c(C=O)c1] |
| 38 | 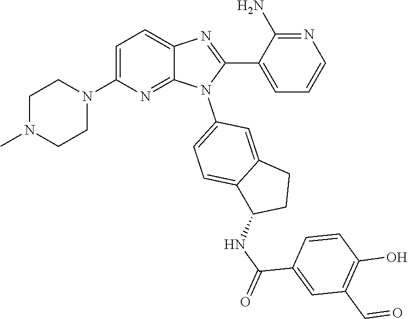 [SMILES: CN1CCN(c2ccc3nc(-c4cccnc4N)n(-c4ccc5c(c4)CC[C@@H]5NC(=O)c4ccc(O)c(C=O)c4)c3n2)CC1] |
| 39 | 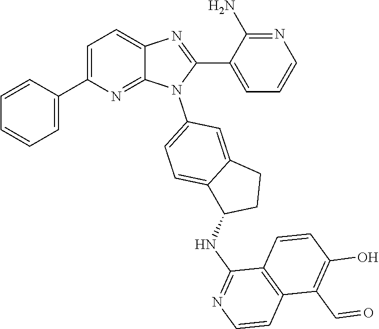 [SMILES: Nc1ncccc1-c1nc2ccc(-c3ccccc3)nc2n1-c1ccc2c(c1)CC[C@@H]2Nc1nccc2c(C=O)c(O)ccc12] |
| 40 | 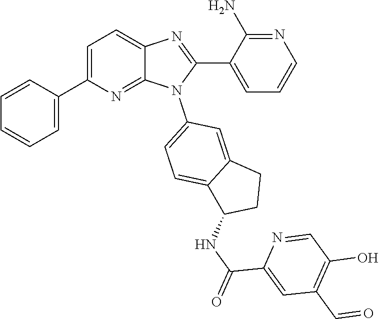 [SMILES: Nc1ncccc1-c1nc2ccc(-c3ccccc3)nc2n1-c1ccc2c(c1)CC[C@@H]2NC(=O)c1cc(C=O)c(O)cn1] |
| 41 | 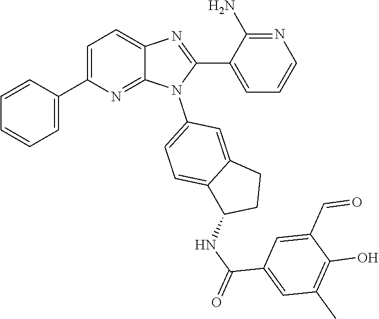 [SMILES: Cc1cc(C(=O)N[C@H]2CCc3cc(-n4c(-c5cccnc5N)nc5ccc(-c6ccccc6)nc54)ccc32)cc(C=O)c1O] |
| 42 | 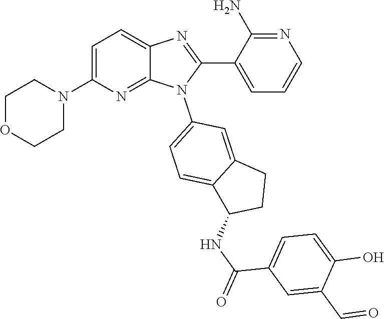 [SMILES: Nc1ncccc1-c1nc2ccc(N3CCOCC3)nc2n1-c1ccc2c(c1)CC[C@@H]2NC(=O)c1ccc(O)c(C=O)c1] |
| 43 | 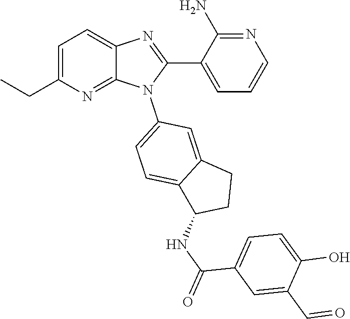 [SMILES: CCc1ccc2nc(-c3cccnc3N)n(-c3ccc4c(c3)CC[C@@H]4NC(=O)c3ccc(O)c(C=O)c3)c2n1] |
| 44 | 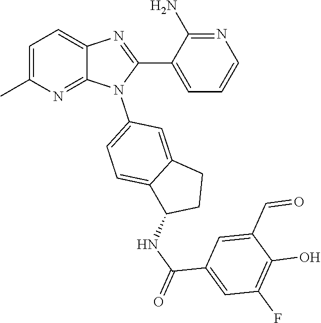 [SMILES: Cc1ccc2nc(-c3cccnc3N)n(-c3ccc4c(c3)CC[C@@H]4NC(=O)c3cc(F)c(O)c(C=O)c3)c2n1] |
| 45 | 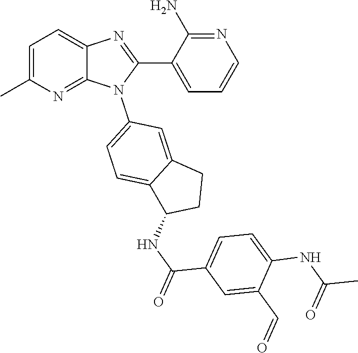 [SMILES: CC(=O)Nc1ccc(C(=O)N[C@H]2CCc3cc(-n4c(-c5cccnc5N)nc5ccc(C)nc54)ccc32)cc1C=O] |
| 46 | 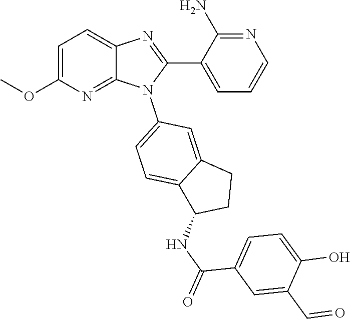 [SMILES: COc1ccc2nc(-c3cccnc3N)n(-c3ccc4c(c3)CC[C@@H]4NC(=O)c3ccc(O)c(C=O)c3)c2n1] |
| 47 | 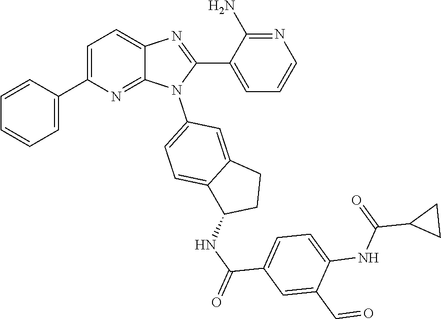 [SMILES: Nc1ncccc1-c1nc2ccc(-c3ccccc3)nc2n1-c1ccc2c(c1)CC[C@@H]2NC(=O)c1ccc(NC(=O)C2CC2)c(C=O)c1] |
| 48 | 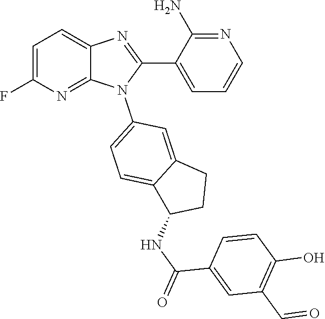 [SMILES: Nc1ncccc1-c1nc2ccc(F)nc2n1-c1ccc2c(c1)CC[C@@H]2NC(=O)c1ccc(O)c(C=O)c1] |
| 49 | 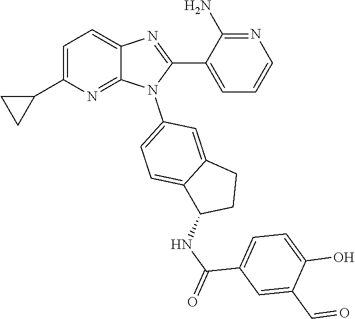 [SMILES: Nc1ncccc1-c1nc2ccc(C3CC3)nc2n1-c1ccc2c(c1)CC[C@@H]2NC(=O)c1ccc(O)c(C=O)c1] |
| 50 | 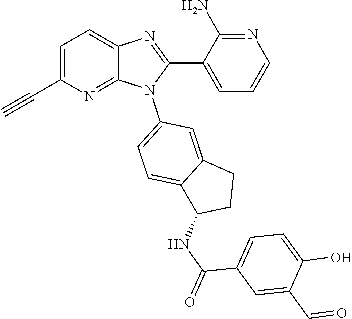 [SMILES: C#Cc1ccc2nc(-c3cccnc3N)n(-c3ccc4c(c3)CC[C@@H]4NC(=O)c3ccc(O)c(C=O)c3)c2n1] |
| 51 | 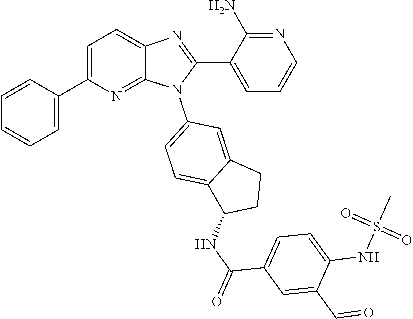 [SMILES: CS(=O)(=O)Nc1ccc(C(=O)N[C@H]2CCc3cc(-n4c(-c5cccnc5N)nc5ccc(-c6ccccc6)nc54)ccc32)cc1C=O] |
| 52 | 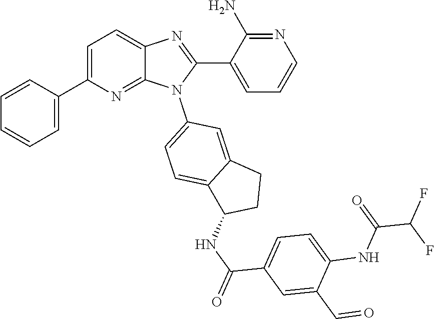 [SMILES: Nc1ncccc1-c1nc2ccc(-c3ccccc3)nc2n1-c1ccc2c(c1)CC[C@@H]2NC(=O)c1ccc(NC(=O)C(F)F)c(C=O)c1] |
| 53 | 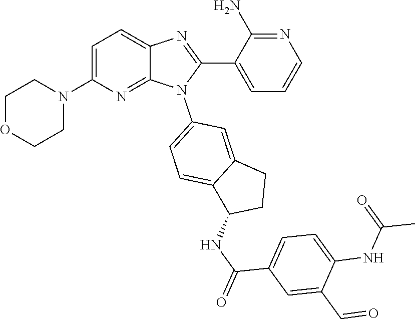 [SMILES: CC(=O)Nc1ccc(C(=O)N[C@H]2CCc3cc(-n4c(-c5cccnc5N)nc5ccc(N6CCOCC6)nc54)ccc32)cc1C=O] |
| 54 | 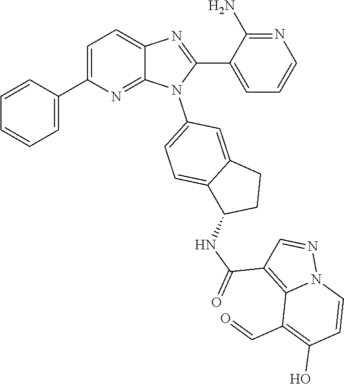 [SMILES: Nc1ncccc1-c1nc2ccc(-c3ccccc3)nc2n1-c1ccc2c(c1)CC[C@@H]2NC(=O)c1cnn2ccc(O)c(C=O)c12] |
| 55 | 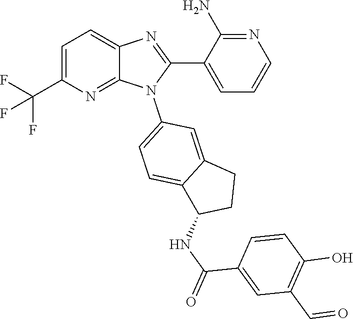 [SMILES: Nc1ncccc1-c1nc2ccc(C(F)(F)F)nc2n1-c1ccc2c(c1)CC[C@@H]2NC(=O)c1ccc(O)c(C=O)c1] |
| 56 | 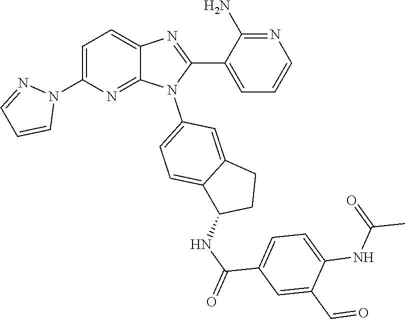 [SMILES: CC(=O)Nc1ccc(C(=O)N[C@H]2CCc3cc(-n4c(-c5cccnc5N)nc5ccc(-n6cccn6)nc54)ccc32)cc1C=O] |
| 57 | 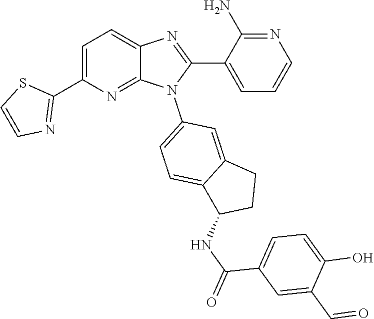 [SMILES: Nc1ncccc1-c1nc2ccc(-c3nccs3)nc2n1-c1ccc2c(c1)CC[C@@H]2NC(=O)c1ccc(O)c(C=O)c1] |
| 58 | 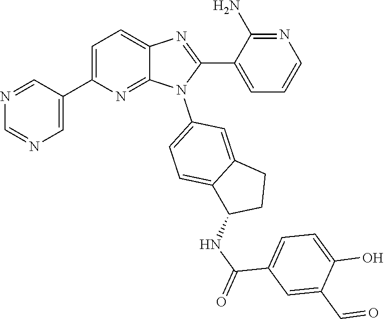 [SMILES: Nc1ncccc1-c1nc2ccc(-c3cncnc3)nc2n1-c1ccc2c(c1)CC[C@@H]2NC(=O)c1ccc(O)c(C=O)c1] |
| 59 | 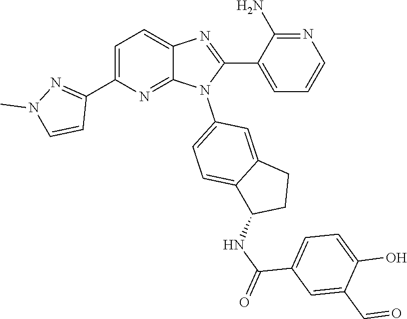 [SMILES: Cn1ccc(-c2ccc3nc(-c4cccnc4N)n(-c4ccc5c(c4)CC[C@@H]5NC(=O)c4ccc(O)c(C=O)c4)c3n2)n1] |
| 60 | 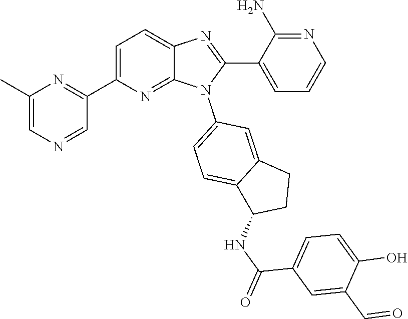 [SMILES: Cc1cncc(-c2ccc3nc(-c4cccnc4N)n(-c4ccc5c(c4)CC[C@@H]5NC(=O)c4ccc(O)c(C=O)c4)c3n2)n1] |
| 61 | 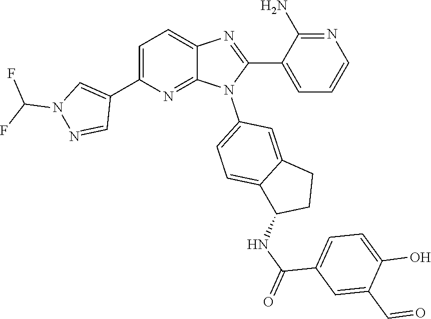 [SMILES: Nc1ncccc1-c1nc2ccc(-c3cnn(C(F)F)c3)nc2n1-c1ccc2c(c1)CC[C@@H]2NC(=O)c1ccc(O)c(C=O)c1] |
| 62 | 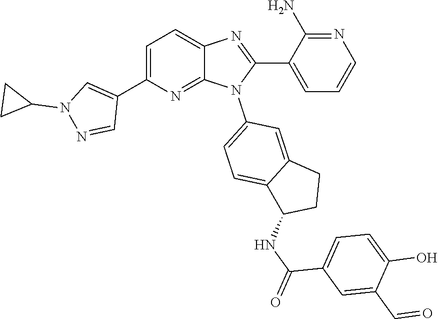 [SMILES: Nc1ncccc1-c1nc2ccc(-c3cnn(C4CC4)c3)nc2n1-c1ccc2c(c1)CC[C@@H]2NC(=O)c1ccc(O)c(C=O)c1] |
| 63 | 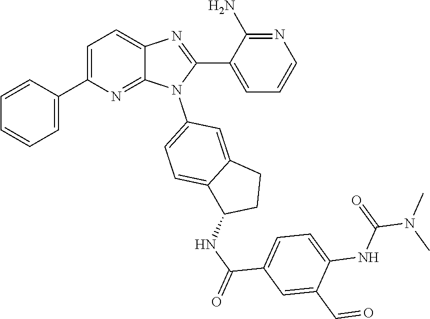 [SMILES: CN(C)C(=O)Nc1ccc(C(=O)N[C@H]2CCc3cc(-n4c(-c5cccnc5N)nc5ccc(-c6ccccc6)nc54)ccc32)cc1C=O] |
| 64 | 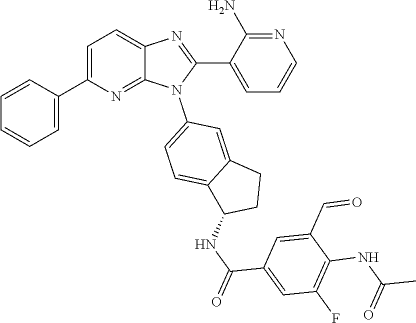 [SMILES: CC(=O)Nc1c(F)cc(C(=O)N[C@H]2CCc3cc(-n4c(-c5cccnc5N)nc5ccc(-c6ccccc6)nc54)ccc32)cc1C=O] |
| 65 | 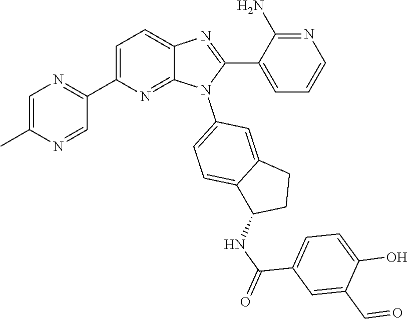 [SMILES: Cc1cnc(-c2ccc3nc(-c4cccnc4N)n(-c4ccc5c(c4)CC[C@@H]5NC(=O)c4ccc(O)c(C=O)c4)c3n2)cn1] |
| 66 | 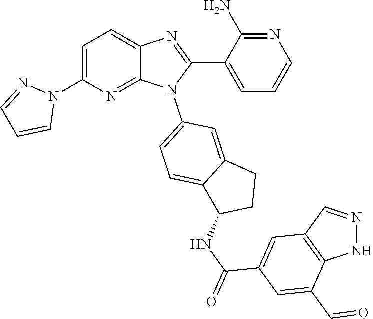 [SMILES: Nc1ncccc1-c1nc2ccc(-n3cccn3)nc2n1-c1ccc2c(c1)CC[C@@H]2NC(=O)c1cc(C=O)c2[nH]ncc2c1] |
| 67 | 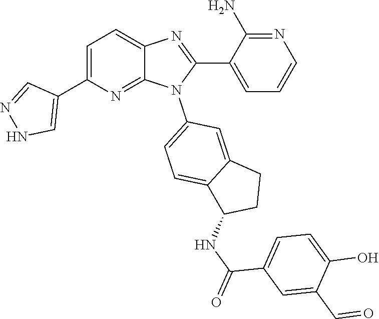 [SMILES: Nc1ncccc1-c1nc2ccc(-c3cn[nH]c3)nc2n1-c1ccc2c(c1)CC[C@@H]2NC(=O)c1ccc(O)c(C=O)c1] |
| 68 | 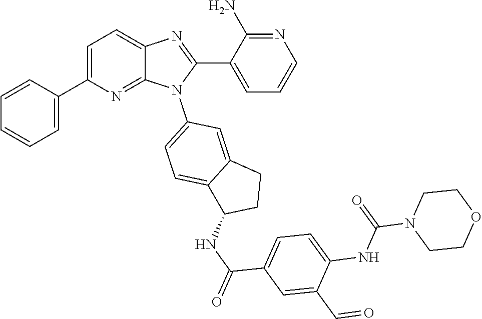 [SMILES: Nc1ncccc1-c1nc2ccc(-c3ccccc3)nc2n1-c1ccc2c(c1)CC[C@@H]2NC(=O)c1ccc(NC(=O)N2CCOCC2)c(C=O)c1] |
| 69 | 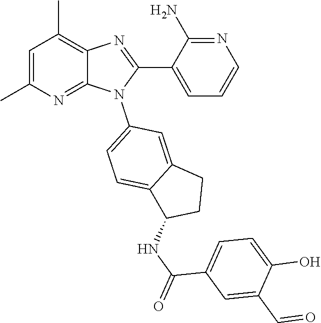 [SMILES: Cc1cc(C)c2nc(-c3cccnc3N)n(-c3ccc4c(c3)CC[C@@H]4NC(=O)c3ccc(O)c(C=O)c3)c2n1] |
| 70 | 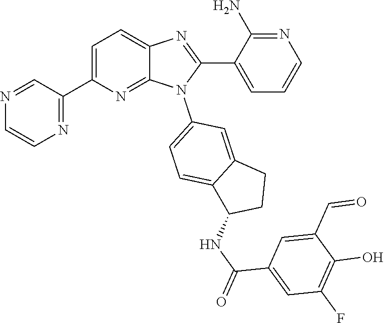 [SMILES: Nc1ncccc1-c1nc2ccc(-c3cnccn3)nc2n1-c1ccc2c(c1)CC[C@@H]2NC(=O)c1cc(F)c(O)c(C=O)c1] |
| 71 | 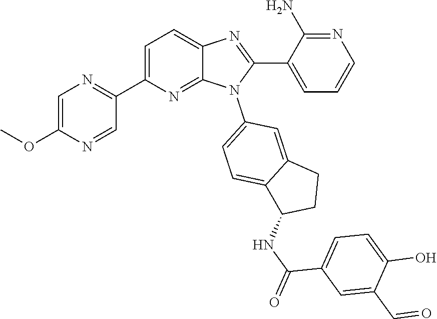 [SMILES: COc1cnc(-c2ccc3nc(-c4cccnc4N)n(-c4ccc5c(c4)CC[C@@H]5NC(=O)c4ccc(O)c(C=O)c4)c3n2)cn1] |
| 72 | 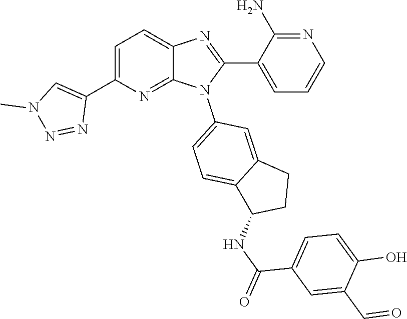 [SMILES: Cn1cc(-c2ccc3nc(-c4cccnc4N)n(-c4ccc5c(c4)CC[C@@H]5NC(=O)c4ccc(O)c(C=O)c4)c3n2)nn1] |
| 73 | 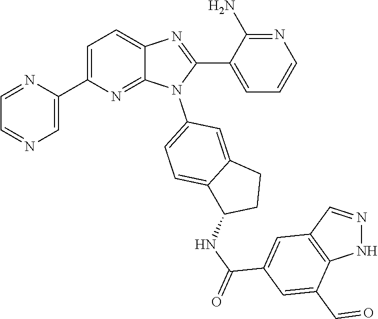 [SMILES: Nc1ncccc1-c1nc2ccc(-c3cnccn3)nc2n1-c1ccc2c(c1)CC[C@@H]2NC(=O)c1cc(C=O)c2[nH]ncc2c1] |
| 74 | 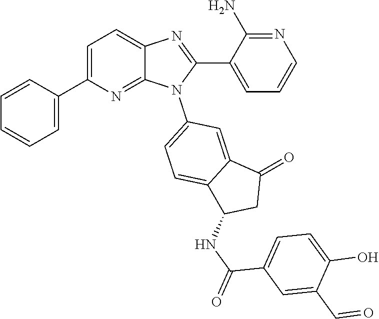 [SMILES: Nc1ncccc1-c1nc2ccc(-c3ccccc3)nc2n1-c1ccc2c(c1)C(=O)C[C@@H]2NC(=O)c1ccc(O)c(C=O)c1] |
| 75 | 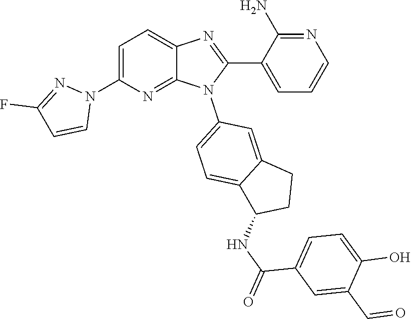 [SMILES: Nc1ncccc1-c1nc2ccc(-n3ccc(F)n3)nc2n1-c1ccc2c(c1)CC[C@@H]2NC(=O)c1ccc(O)c(C=O)c1] |
| 76 | 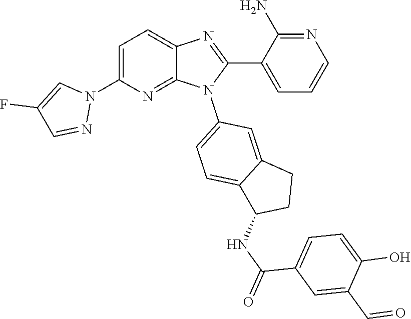 [SMILES: Nc1ncccc1-c1nc2ccc(-n3cc(F)cn3)nc2n1-c1ccc2c(c1)CC[C@@H]2NC(=O)c1ccc(O)c(C=O)c1] |
| 77 | 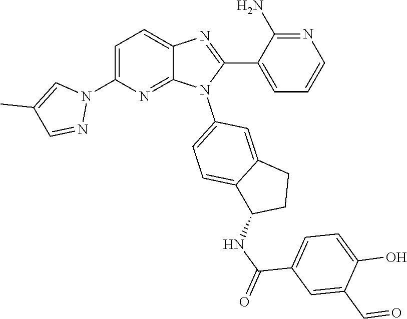 [SMILES: Cc1cnn(-c2ccc3nc(-c4cccnc4N)n(-c4ccc5c(c4)CC[C@@H]5NC(=O)c4ccc(O)c(C=O)c4)c3n2)c1] |
| 78 | 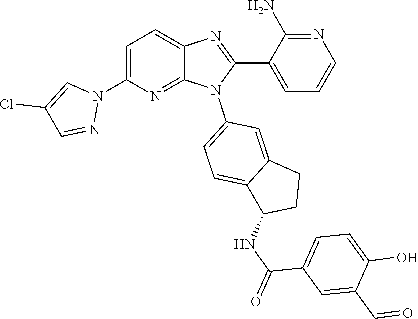 [SMILES: Nc1ncccc1-c1nc2ccc(-n3cc(Cl)cn3)nc2n1-c1ccc2c(c1)CC[C@@H]2NC(=O)c1ccc(O)c(C=O)c1] |
| 79 | 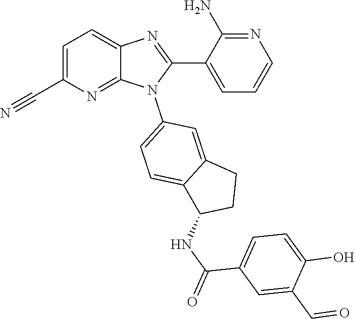 [SMILES: N#Cc1ccc2nc(-c3cccnc3N)n(-c3ccc4c(c3)CC[C@@H]4NC(=O)c3ccc(O)c(C=O)c3)c2n1] |
| 80 | 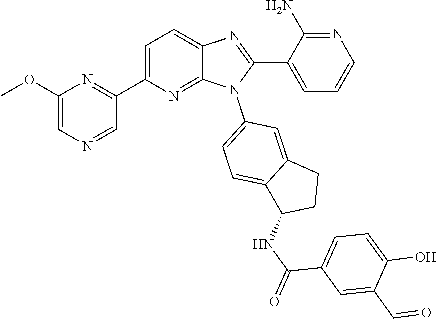 [SMILES: COc1cncc(-c2ccc3nc(-c4cccnc4N)n(-c4ccc5c(c4)CC[C@@H]5NC(=O)c4ccc(O)c(C=O)c4)c3n2)n1] |
| 81 | 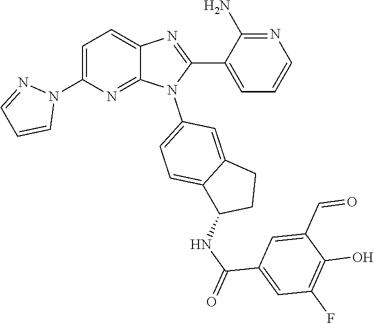 [SMILES: Nc1ncccc1-c1nc2ccc(-n3cccn3)nc2n1-c1ccc2c(c1)CC[C@@H]2NC(=O)c1cc(F)c(O)c(C=O)c1] |
| 82 | 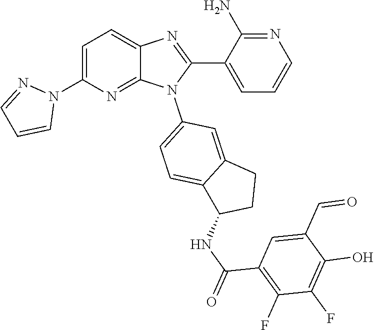 [SMILES: Nc1ncccc1-c1nc2ccc(-n3cccn3)nc2n1-c1ccc2c(c1)CC[C@@H]2NC(=O)c1cc(C=O)c(O)c(F)c1F] |
| 83 | 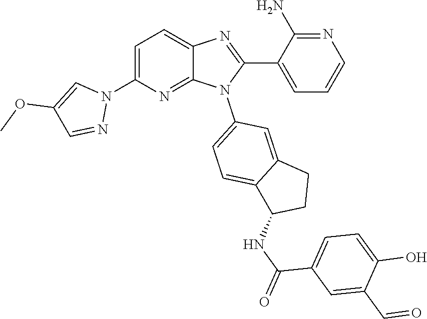 [SMILES: COc1cnn(-c2ccc3nc(-c4cccnc4N)n(-c4ccc5c(c4)CC[C@@H]5NC(=O)c4ccc(O)c(C=O)c4)c3n2)c1] |
| 84 | 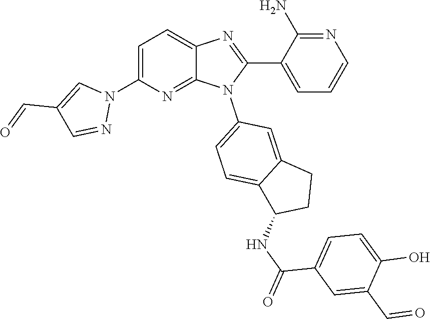 [SMILES: Nc1ncccc1-c1nc2ccc(-n3cc(C=O)cn3)nc2n1-c1ccc2c(c1)CC[C@@H]2NC(=O)c1ccc(O)c(C=O)c1] |
| 85 | 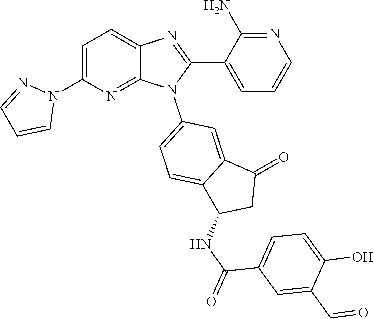 [SMILES: Nc1ncccc1-c1nc2ccc(-n3cccn3)nc2n1-c1ccc2c(c1)C(=O)C[C@@H]2NC(=O)c1ccc(O)c(C=O)c1] |
| 86 | 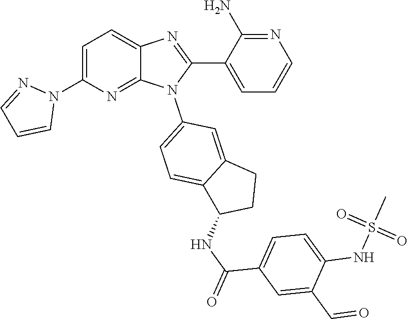 [SMILES: CS(=O)(=O)Nc1ccc(C(=O)N[C@H]2CCc3cc(-n4c(-c5cccnc5N)nc5ccc(-n6cccn6)nc54)ccc32)cc1C=O] |
| 87 | 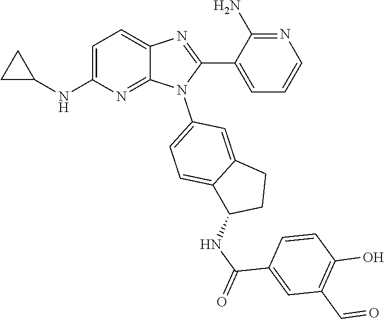 [SMILES: Nc1ncccc1-c1nc2ccc(NC3CC3)nc2n1-c1ccc2c(c1)CC[C@@H]2NC(=O)c1ccc(O)c(C=O)c1] |
| 88 | 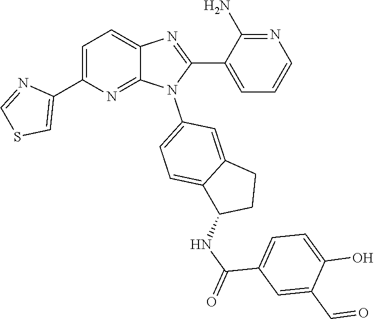 [SMILES: Nc1ncccc1-c1nc2ccc(-c3cscn3)nc2n1-c1ccc2c(c1)CC[C@@H]2NC(=O)c1ccc(O)c(C=O)c1] |
| 89 | 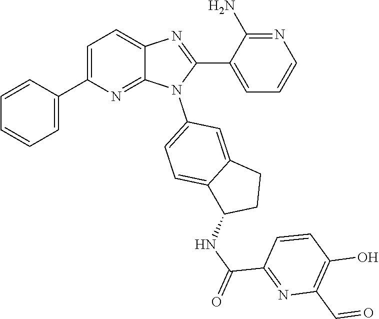 [SMILES: Nc1ncccc1-c1nc2ccc(-c3ccccc3)nc2n1-c1ccc2c(c1)CC[C@@H]2NC(=O)c1ccc(O)c(C=O)n1] |
| 90 | 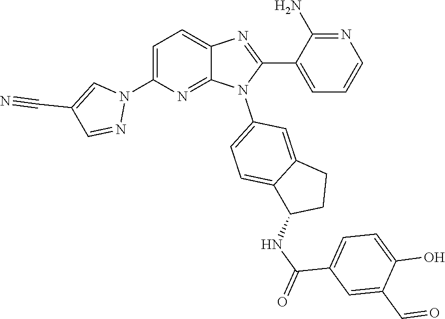 [SMILES: N#Cc1cnn(-c2ccc3nc(-c4cccnc4N)n(-c4ccc5c(c4)CC[C@@H]5NC(=O)c4ccc(O)c(C=O)c4)c3n2)c1] |
| 91 | 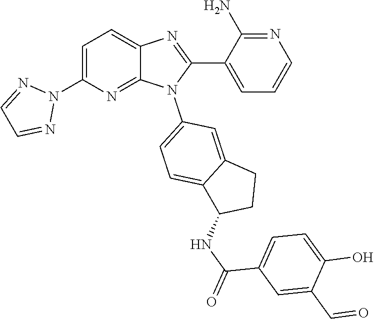 [SMILES: Nc1ncccc1-c1nc2ccc(-n3nccn3)nc2n1-c1ccc2c(c1)CC[C@@H]2NC(=O)c1ccc(O)c(C=O)c1] |
| 92 | 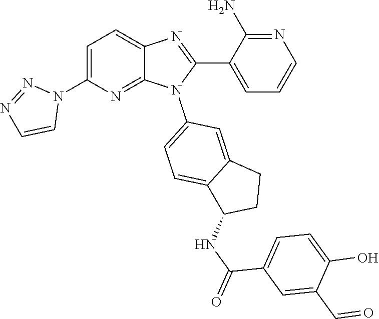 [SMILES: Nc1ncccc1-c1nc2ccc(-n3ccnn3)nc2n1-c1ccc2c(c1)CC[C@@H]2NC(=O)c1ccc(O)c(C=O)c1] |
| 93 | 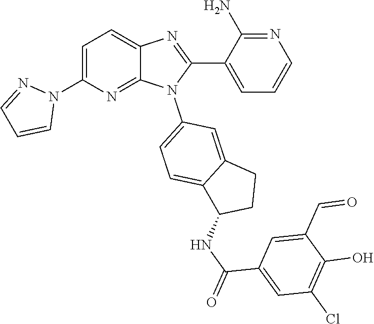 [SMILES: Nc1ncccc1-c1nc2ccc(-n3cccn3)nc2n1-c1ccc2c(c1)CC[C@@H]2NC(=O)c1cc(Cl)c(O)c(C=O)c1] |
| 94 | 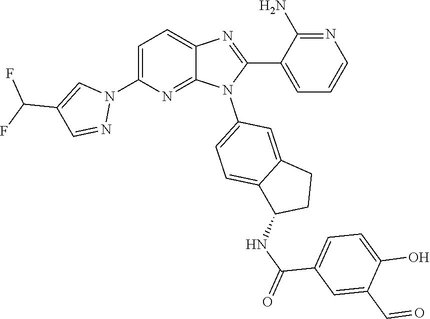 [SMILES: Nc1ncccc1-c1nc2ccc(-n3cc(C(F)F)cn3)nc2n1-c1ccc2c(c1)CC[C@@H]2NC(=O)c1ccc(O)c(C=O)c1] |
| 95 | 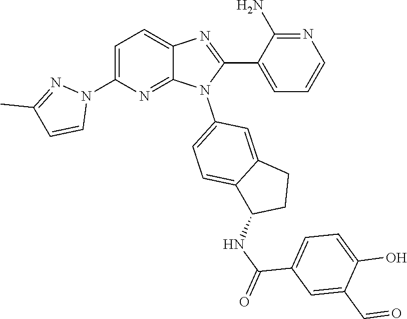 [SMILES: Cc1ccn(-c2ccc3nc(-c4cccnc4N)n(-c4ccc5c(c4)CC[C@@H]5NC(=O)c4ccc(O)c(C=O)c4)c3n2)n1] |
| 96 | 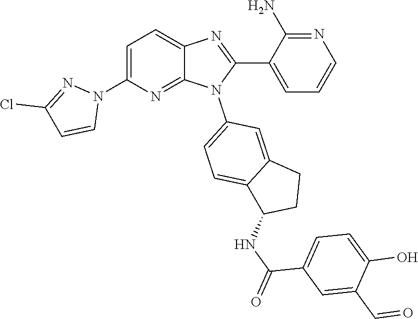 [SMILES: Nc1ncccc1-c1nc2ccc(-n3ccc(Cl)n3)nc2n1-c1ccc2c(c1)CC[C@@H]2NC(=O)c1ccc(O)c(C=O)c1] |
| 97 | 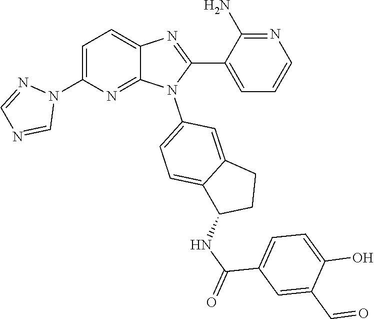 [SMILES: Nc1ncccc1-c1nc2ccc(-n3cncn3)nc2n1-c1ccc2c(c1)CC[C@@H]2NC(=O)c1ccc(O)c(C=O)c1] |
| 98 | 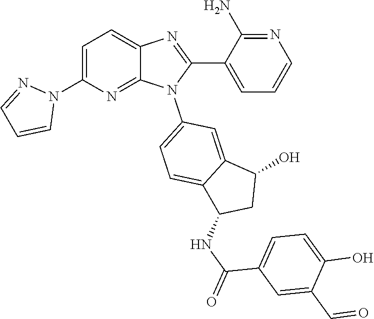 [SMILES: Nc1ncccc1-c1nc2ccc(-n3cccn3)nc2n1-c1ccc2c(c1)[C@H](O)C[C@@H]2NC(=O)c1ccc(O)c(C=O)c1] |
| 99 | 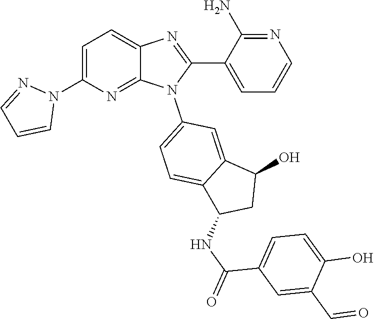 [SMILES: Nc1ncccc1-c1nc2ccc(-n3cccn3)nc2n1-c1ccc2c(c1)[C@@H](O)C[C@@H]2NC(=O)c1ccc(O)c(C=O)c1] |
| 100 | 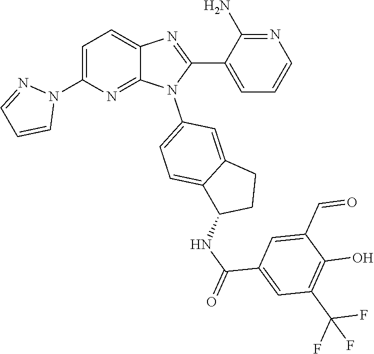 [SMILES: Nc1ncccc1-c1nc2ccc(-n3cccn3)nc2n1-c1ccc2c(c1)CC[C@@H]2NC(=O)c1cc(C=O)c(O)c(C(F)(F)F)c1] |
| 101 | 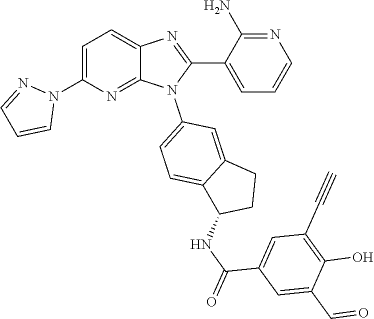 [SMILES: C#Cc1cc(C(=O)N[C@H]2CCc3cc(-n4c(-c5cccnc5N)nc5ccc(-n6cccn6)nc54)ccc32)cc(C=O)c1O] |
| 102 | 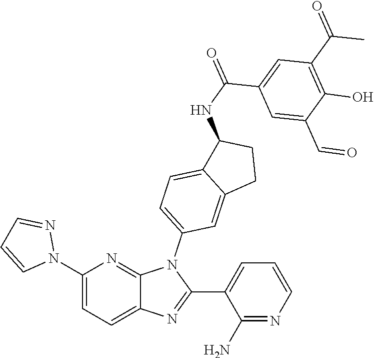 [SMILES: CC(=O)c1cc(C(=O)N[C@H]2CCc3cc(-n4c(-c5cccnc5N)nc5ccc(-n6cccn6)nc54)ccc32)cc(C=O)c1O] |
| 103 | 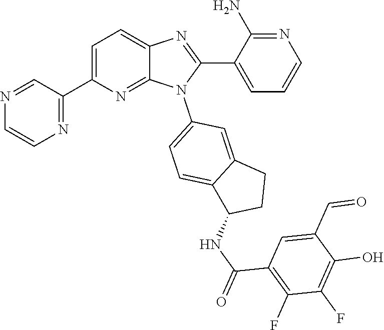 [SMILES: Nc1ncccc1-c1nc2ccc(-c3cnccn3)nc2n1-c1ccc2c(c1)CC[C@@H]2NC(=O)c1cc(C=O)c(O)c(F)c1F] |
| 104 | 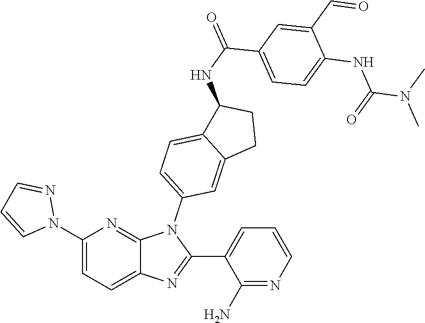 [SMILES: CN(C)C(=O)Nc1ccc(C(=O)N[C@H]2CCc3cc(-n4c(-c5cccnc5N)nc5ccc(-n6cccn6)nc54)ccc32)cc1C=O] |
| 105 | 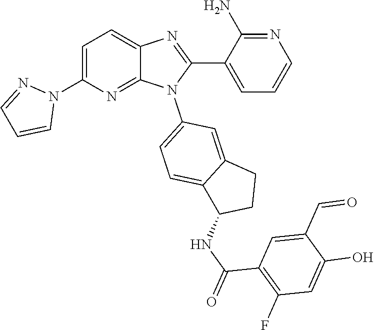 [SMILES: Nc1ncccc1-c1nc2ccc(-n3cccn3)nc2n1-c1ccc2c(c1)CC[C@@H]2NC(=O)c1cc(C=O)c(O)cc1F] |
| 106 | 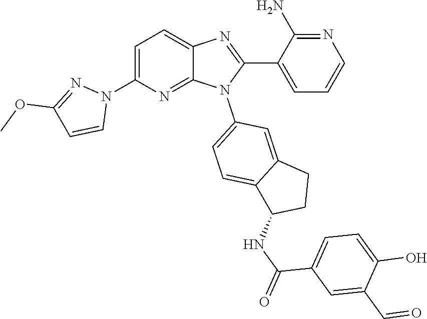 [SMILES: COc1ccn(-c2ccc3nc(-c4cccnc4N)n(-c4ccc5c(c4)CC[C@@H]5NC(=O)c4ccc(O)c(C=O)c4)c3n2)n1] |
| 107 | 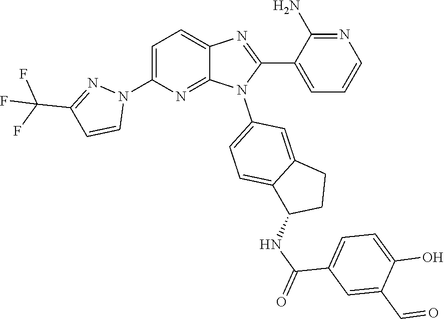 [SMILES: Nc1ncccc1-c1nc2ccc(-n3ccc(C(F)(F)F)n3)nc2n1-c1ccc2c(c1)CC[C@@H]2NC(=O)c1ccc(O)c(C=O)c1] |
| 108 | 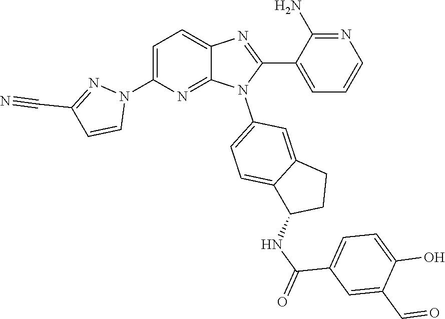 [SMILES: N#Cc1ccn(-c2ccc3nc(-c4cccnc4N)n(-c4ccc5c(c4)CC[C@@H]5NC(=O)c4ccc(O)c(C=O)c4)c3n2)n1] |
| 109 | 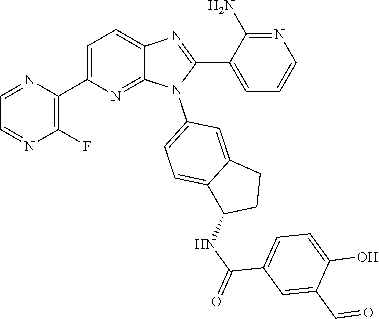 [SMILES: Nc1ncccc1-c1nc2ccc(-c3nccnc3F)nc2n1-c1ccc2c(c1)CC[C@@H]2NC(=O)c1ccc(O)c(C=O)c1] |
| 110 | 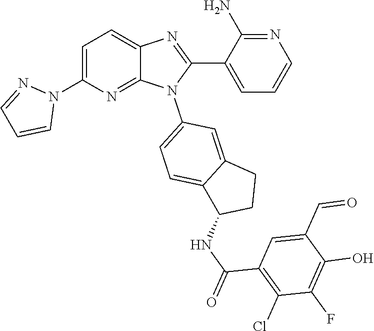 [SMILES: Nc1ncccc1-c1nc2ccc(-n3cccn3)nc2n1-c1ccc2c(c1)CC[C@@H]2NC(=O)c1cc(C=O)c(O)c(F)c1Cl] |
| 111 | 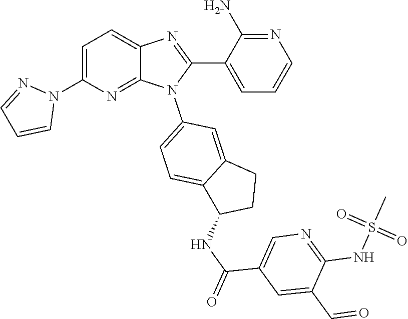 [SMILES: CS(=O)(=O)Nc1ncc(C(=O)N[C@H]2CCc3cc(-n4c(-c5cccnc5N)nc5ccc(-n6cccn6)nc54)ccc32)cc1C=O] |
| 112 | 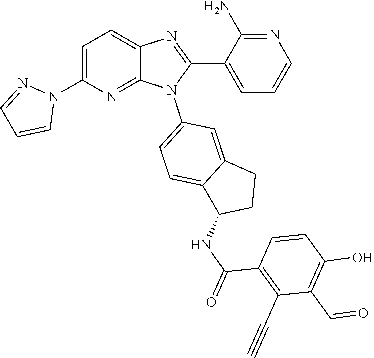 [SMILES: C#Cc1c(C(=O)N[C@H]2CCc3cc(-n4c(-c5cccnc5N)nc5ccc(-n6cccn6)nc54)ccc32)ccc(O)c1C=O] |
| 113 | 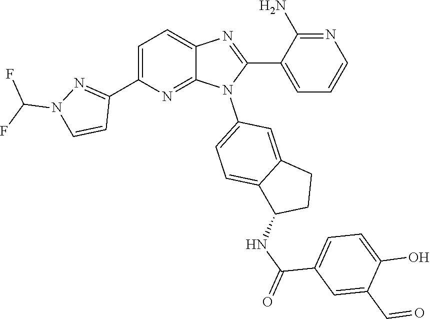 [SMILES: Nc1ncccc1-c1nc2ccc(-c3ccn(C(F)F)n3)nc2n1-c1ccc2c(c1)CC[C@@H]2NC(=O)c1ccc(O)c(C=O)c1] |
| 114 | 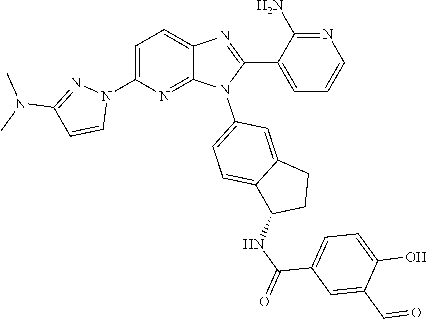 [SMILES: CN(C)c1ccn(-c2ccc3nc(-c4cccnc4N)n(-c4ccc5c(c4)CC[C@@H]5NC(=O)c4ccc(O)c(C=O)c4)c3n2)n1] |
| 115 | 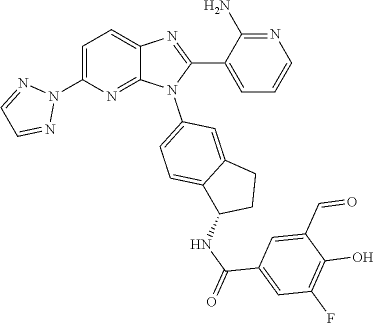 [SMILES: Nc1ncccc1-c1nc2ccc(-n3nccn3)nc2n1-c1ccc2c(c1)CC[C@@H]2NC(=O)c1cc(F)c(O)c(C=O)c1] |
| 116 | 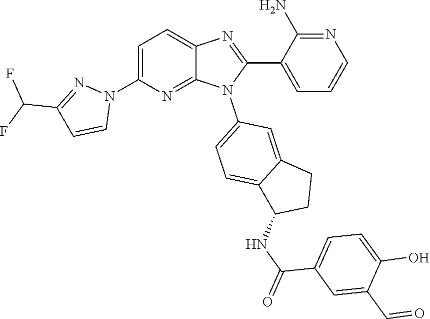 [SMILES: Nc1ncccc1-c1nc2ccc(-n3ccc(C(F)F)n3)nc2n1-c1ccc2c(c1)CC[C@@H]2NC(=O)c1ccc(O)c(C=O)c1] |
| 117 | 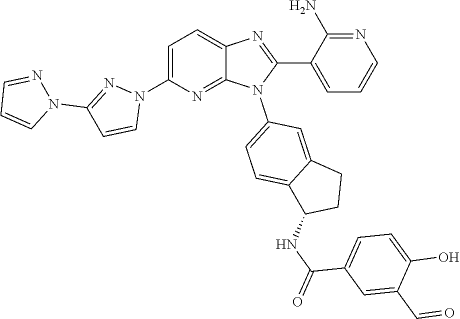 [SMILES: Nc1ncccc1-c1nc2ccc(-n3ccc(-n4cccn4)n3)nc2n1-c1ccc2c(c1)CC[C@@H]2NC(=O)c1ccc(O)c(C=O)c1] |
| 118 | 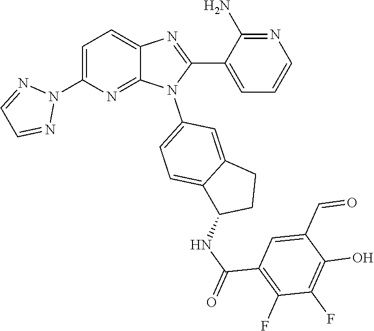 [SMILES: Nc1ncccc1-c1nc2ccc(-n3nccn3)nc2n1-c1ccc2c(c1)CC[C@@H]2NC(=O)c1cc(C=O)c(O)c(F)c1F] |
| 119 | 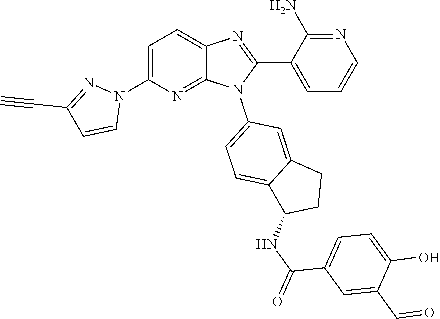 [SMILES: C#Cc1ccn(-c2ccc3nc(-c4cccnc4N)n(-c4ccc5c(c4)CC[C@@H]5NC(=O)c4ccc(O)c(C=O)c4)c3n2)n1] |
| 120 | 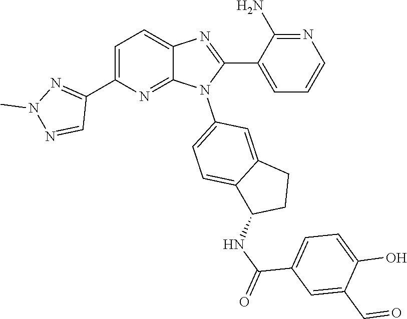 [SMILES: Cn1ncc(-c2ccc3nc(-c4cccnc4N)n(-c4ccc5c(c4)CC[C@@H]5NC(=O)c4ccc(O)c(C=O)c4)c3n2)n1] |
| 121 | 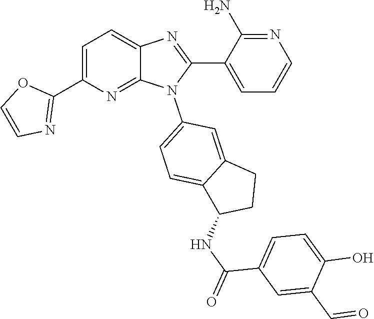 [SMILES: Nc1ncccc1-c1nc2ccc(-c3ncco3)nc2n1-c1ccc2c(c1)CC[C@@H]2NC(=O)c1ccc(O)c(C=O)c1] |
| 122 | 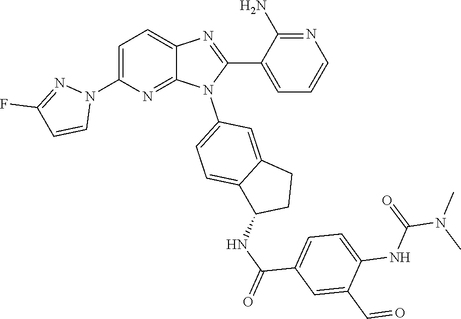 [SMILES: CN(C)C(=O)Nc1ccc(C(=O)N[C@H]2CCc3cc(-n4c(-c5cccnc5N)nc5ccc(-n6ccc(F)n6)nc54)ccc32)cc1C=O] |
| 123 | 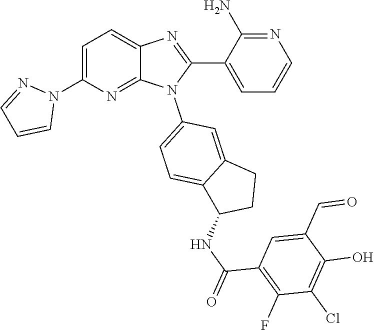 [SMILES: Nc1ncccc1-c1nc2ccc(-n3cccn3)nc2n1-c1ccc2c(c1)CC[C@@H]2NC(=O)c1cc(C=O)c(O)c(Cl)c1F] |
| 124 | 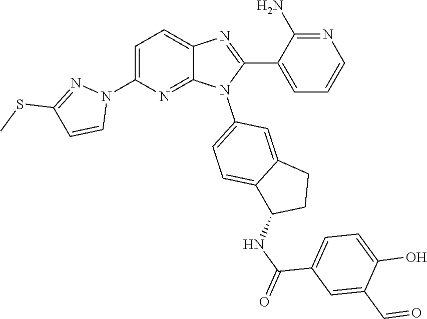 [SMILES: CSc1ccn(-c2ccc3nc(-c4cccnc4N)n(-c4ccc5c(c4)CC[C@@H]5NC(=O)c4ccc(O)c(C=O)c4)c3n2)n1] |
| 125 | 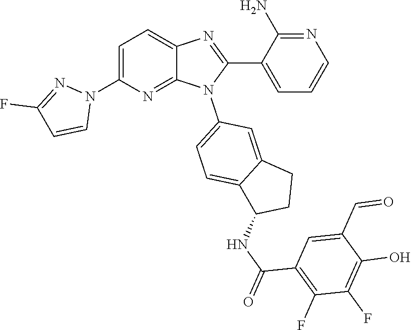 [SMILES: Nc1ncccc1-c1nc2ccc(-n3ccc(F)n3)nc2n1-c1ccc2c(c1)CC[C@@H]2NC(=O)c1cc(C=O)c(O)c(F)c1F] |
| 126 | 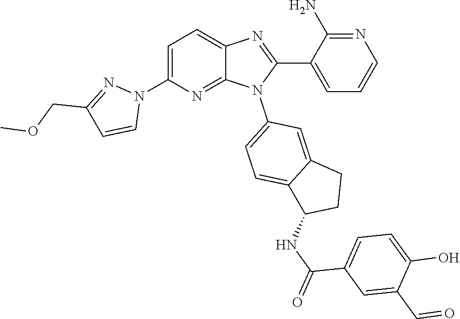 [SMILES: COCc1ccn(-c2ccc3nc(-c4cccnc4N)n(-c4ccc5c(c4)CC[C@@H]5NC(=O)c4ccc(O)c(C=O)c4)c3n2)n1] |
| 127 | 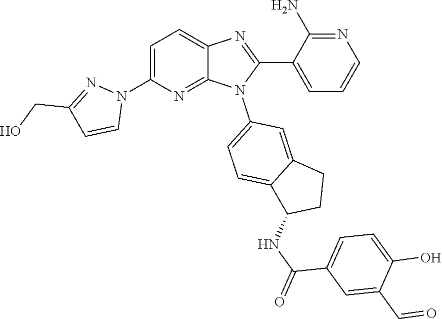 [SMILES: Nc1ncccc1-c1nc2ccc(-n3ccc(CO)n3)nc2n1-c1ccc2c(c1)CC[C@@H]2NC(=O)c1ccc(O)c(C=O)c1] |
| 128 | 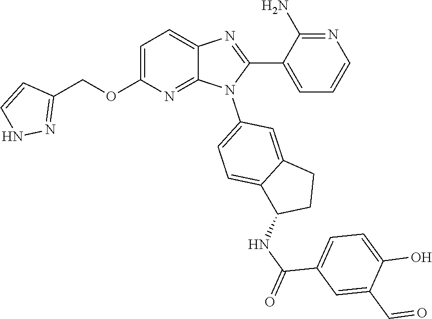 [SMILES: Nc1ncccc1-c1nc2ccc(OCc3cc[nH]n3)nc2n1-c1ccc2c(c1)CC[C@@H]2NC(=O)c1ccc(O)c(C=O)c1] |
| 129 | 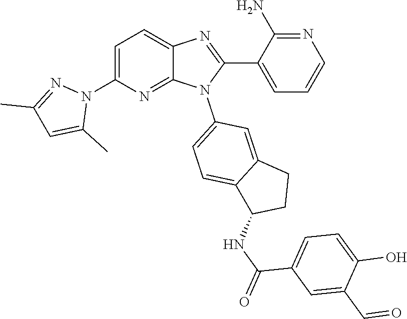 [SMILES: Cc1cc(C)n(-c2ccc3nc(-c4cccnc4N)n(-c4ccc5c(c4)CC[C@@H]5NC(=O)c4ccc(O)c(C=O)c4)c3n2)n1] |
| 130 | 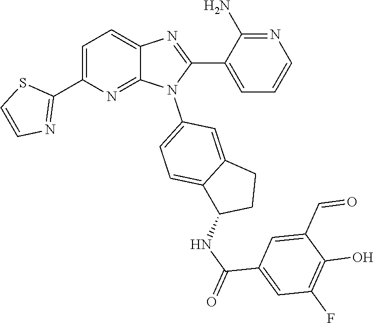 [SMILES: Nc1ncccc1-c1nc2ccc(-c3nccs3)nc2n1-c1ccc2c(c1)CC[C@@H]2NC(=O)c1cc(F)c(O)c(C=O)c1] |
| 131 | 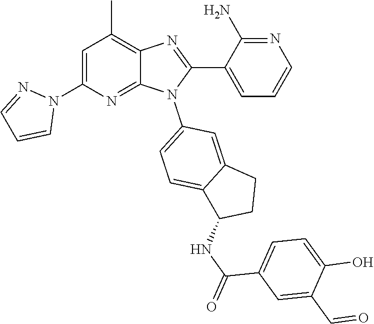 [SMILES: Cc1cc(-n2cccn2)nc2c1nc(-c1cccnc1N)n2-c1ccc2c(c1)CC[C@@H]2NC(=O)c1ccc(O)c(C=O)c1] |
| 132 | 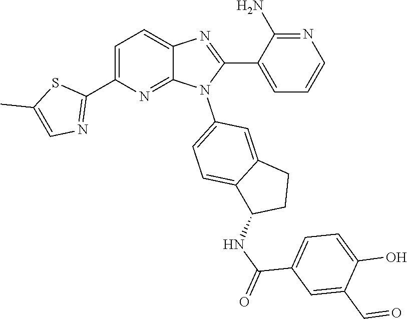 [SMILES: Cc1cnc(-c2ccc3nc(-c4cccnc4N)n(-c4ccc5c(c4)CC[C@@H]5NC(=O)c4ccc(O)c(C=O)c4)c3n2)s1] |
| 133 | 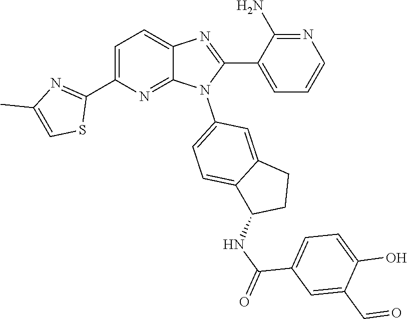 [SMILES: Cc1csc(-c2ccc3nc(-c4cccnc4N)n(-c4ccc5c(c4)CC[C@@H]5NC(=O)c4ccc(O)c(C=O)c4)c3n2)n1] |
| 134 | 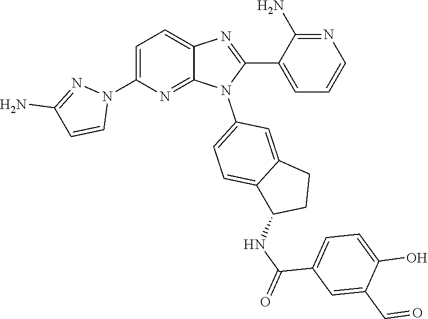 [SMILES: Nc1ccn(-c2ccc3nc(-c4cccnc4N)n(-c4ccc5c(c4)CC[C@@H]5NC(=O)c4ccc(O)c(C=O)c4)c3n2)n1] |
| 135 | 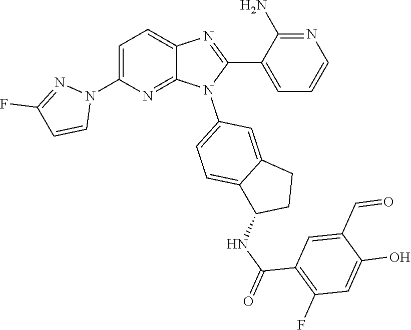 [SMILES: Nc1ncccc1-c1nc2ccc(-n3ccc(F)n3)nc2n1-c1ccc2c(c1)CC[C@@H]2NC(=O)c1cc(C=O)c(O)cc1F] |
| 136 | 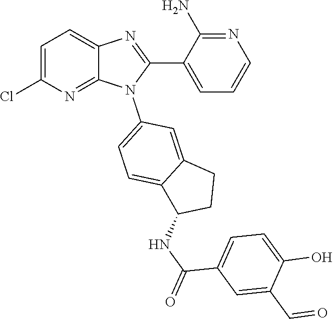 [SMILES: Nc1ncccc1-c1nc2ccc(Cl)nc2n1-c1ccc2c(c1)CC[C@@H]2NC(=O)c1ccc(O)c(C=O)c1] |
| 137 | 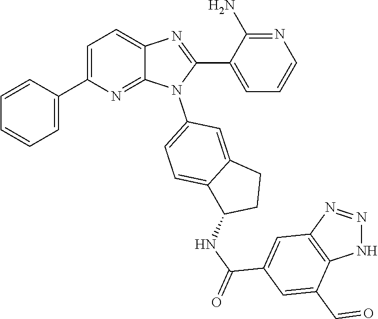 [SMILES: Nc1ncccc1-c1nc2ccc(-c3ccccc3)nc2n1-c1ccc2c(c1)CC[C@@H]2NC(=O)c1cc(C=O)c2[nH]nnc2c1] |
| 138 | 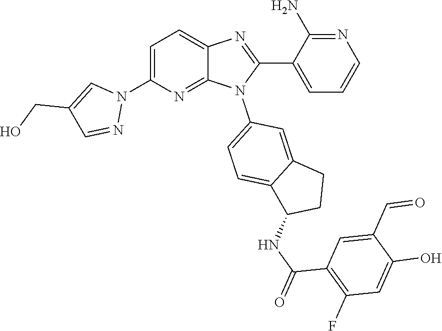 [SMILES: Nc1ncccc1-c1nc2ccc(-n3cc(CO)cn3)nc2n1-c1ccc2c(c1)CC[C@@H]2NC(=O)c1cc(C=O)c(O)cc1F] |
| 139 | 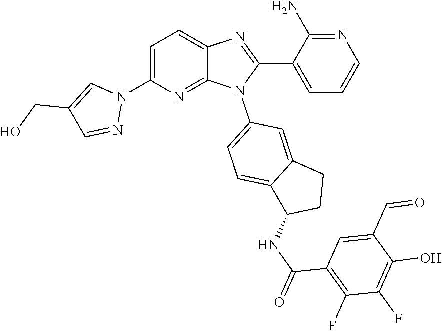 [SMILES: Nc1ncccc1-c1nc2ccc(-n3cc(CO)cn3)nc2n1-c1ccc2c(c1)CC[C@@H]2NC(=O)c1cc(C=O)c(O)c(F)c1F] |
| 140 | 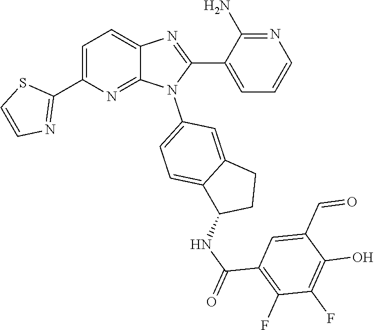 [SMILES: Nc1ncccc1-c1nc2ccc(-c3nccs3)nc2n1-c1ccc2c(c1)CC[C@@H]2NC(=O)c1cc(C=O)c(O)c(F)c1F] |
| 141 | 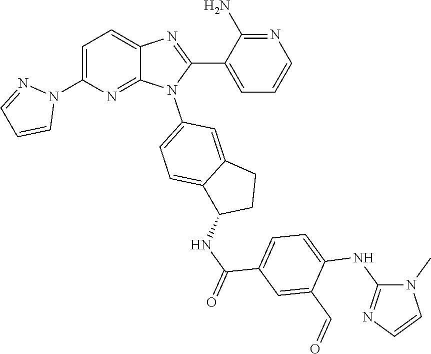 [SMILES: Cn1ccnc1Nc1ccc(C(=O)N[C@H]2CCc3cc(-n4c(-c5cccnc5N)nc5ccc(-n6cccn6)nc54)ccc32)cc1C=O] |
| 142 | 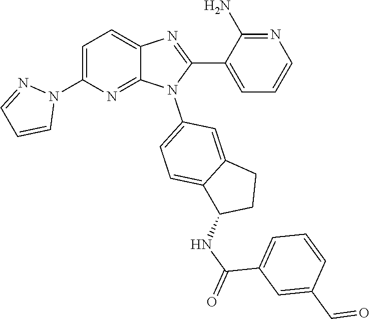 [SMILES: Nc1ncccc1-c1nc2ccc(-n3cccn3)nc2n1-c1ccc2c(c1)CC[C@@H]2NC(=O)c1cccc(C=O)c1] |
| 143 | 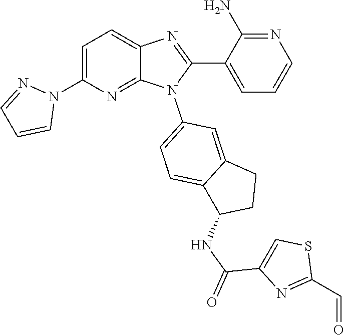 [SMILES: Nc1ncccc1-c1nc2ccc(-n3cccn3)nc2n1-c1ccc2c(c1)CC[C@@H]2NC(=O)c1csc(C=O)n1] |
| 144 | 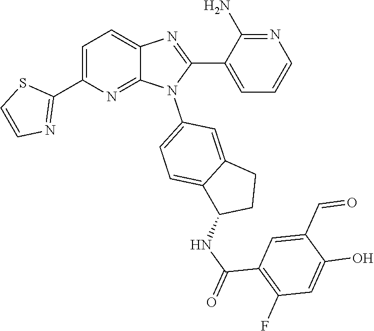 [SMILES: Nc1ncccc1-c1nc2ccc(-c3nccs3)nc2n1-c1ccc2c(c1)CC[C@@H]2NC(=O)c1cc(C=O)c(O)cc1F] |
| 145 | 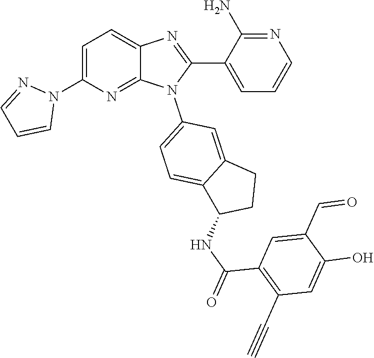 [SMILES: C#Cc1cc(O)c(C=O)cc1C(=O)N[C@H]1CCc2cc(-n3c(-c4cccnc4N)nc4ccc(-n5cccn5)nc43)ccc21] |
| 146 | 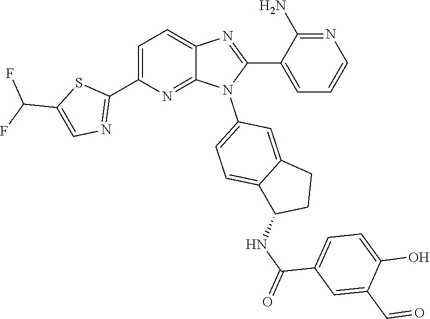 [SMILES: Nc1ncccc1-c1nc2ccc(-c3ncc(C(F)F)s3)nc2n1-c1ccc2c(c1)CC[C@@H]2NC(=O)c1ccc(O)c(C=O)c1] |
| 147 | 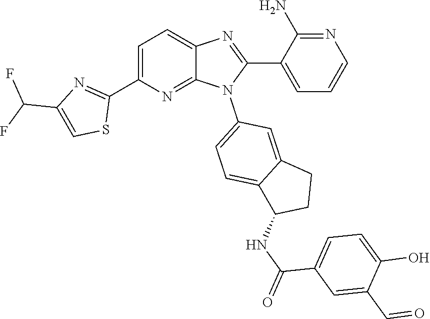 [SMILES: Nc1ncccc1-c1nc2ccc(-c3nc(C(F)F)cs3)nc2n1-c1ccc2c(c1)CC[C@@H]2NC(=O)c1ccc(O)c(C=O)c1] |
| 148 | 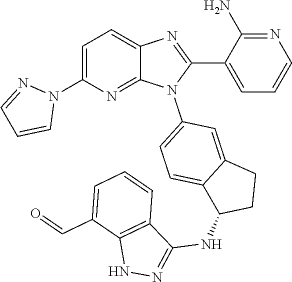 [SMILES: Nc1ncccc1-c1nc2ccc(-n3cccn3)nc2n1-c1ccc2c(c1)CC[C@@H]2Nc1n[nH]c2c(C=O)cccc12] |
| 149 | 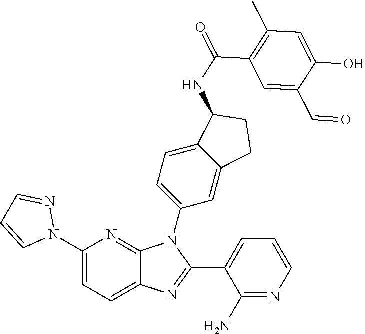 [SMILES: Cc1cc(O)c(C=O)cc1C(=O)N[C@H]1CCc2cc(-n3c(-c4cccnc4N)nc4ccc(-n5cccn5)nc43)ccc21] |
| 150 | 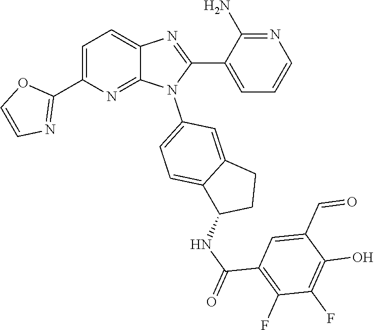 [SMILES: Nc1ncccc1-c1nc2ccc(-c3ncco3)nc2n1-c1ccc2c(c1)CC[C@@H]2NC(=O)c1cc(C=O)c(O)c(F)c1F] |
| 151 | 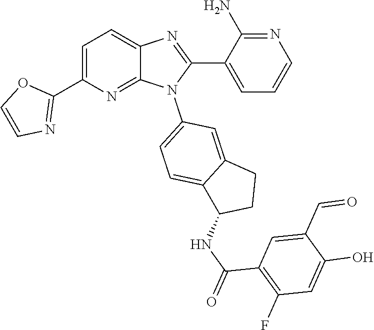 [SMILES: Nc1ncccc1-c1nc2ccc(-c3ncco3)nc2n1-c1ccc2c(c1)CC[C@@H]2NC(=O)c1cc(C=O)c(O)cc1F] |
| 152 | 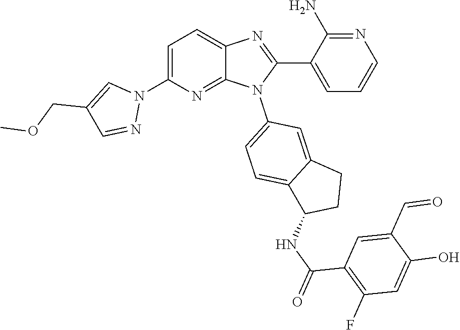 [SMILES: COCc1cnn(-c2ccc3nc(-c4cccnc4N)n(-c4ccc5c(c4)CC[C@@H]5NC(=O)c4cc(C=O)c(O)cc4F)c3n2)c1] |
| 153 | 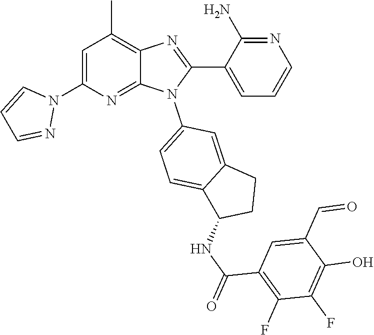 [SMILES: Cc1cc(-n2cccn2)nc2c1nc(-c1cccnc1N)n2-c1ccc2c(c1)CC[C@@H]2NC(=O)c1cc(C=O)c(O)c(F)c1F] |
| 154 | 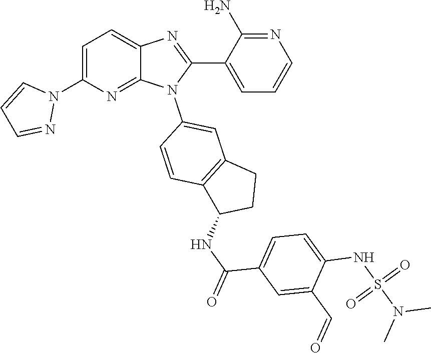 [SMILES: CN(C)S(=O)(=O)Nc1ccc(C(=O)N[C@H]2CCc3cc(-n4c(-c5cccnc5N)nc5ccc(-n6cccn6)nc54)ccc32)cc1C=O] |
| 155 | 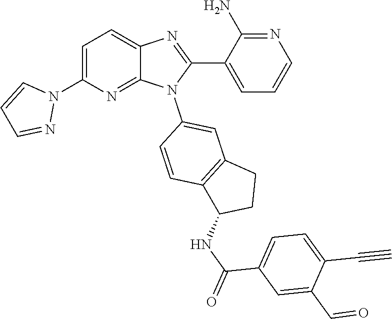 [SMILES: C#Cc1ccc(C(=O)N[C@H]2CCc3cc(-n4c(-c5cccnc5N)nc5ccc(-n6cccn6)nc54)ccc32)cc1C=O] |
| 156 | 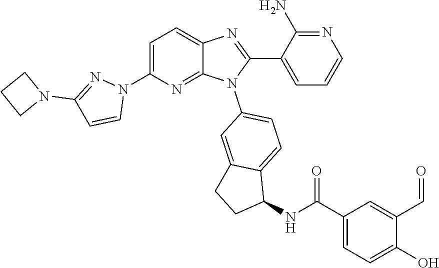 [SMILES: Nc1ncccc1-c1nc2ccc(-n3ccc(N4CCC4)n3)nc2n1-c1ccc2c(c1)CC[C@@H]2NC(=O)c1ccc(O)c(C=O)c1] |
| 157 | 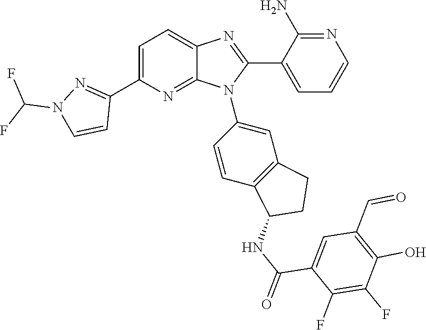 [SMILES: Nc1ncccc1-c1nc2ccc(-c3ccn(C(F)F)n3)nc2n1-c1ccc2c(c1)CC[C@@H]2NC(=O)c1cc(C=O)c(O)c(F)c1F] |
| 158 | 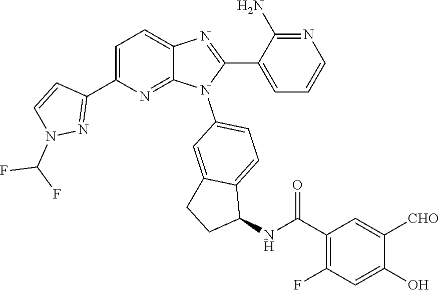 [SMILES: Nc1ncccc1-c1nc2ccc(-c3ccn(C(F)F)n3)nc2n1-c1ccc2c(c1)CC[C@@H]2NC(=O)c1cc(C=O)c(O)cc1F] |
| 159 | 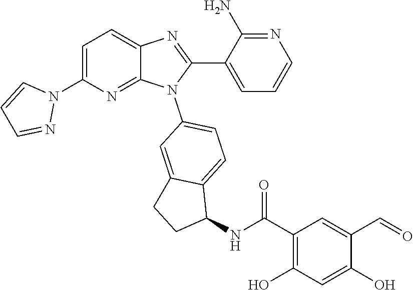 [SMILES: Nc1ncccc1-c1nc2ccc(-n3cccn3)nc2n1-c1ccc2c(c1)CC[C@@H]2NC(=O)c1cc(C=O)c(O)cc1O] |
| 160 | 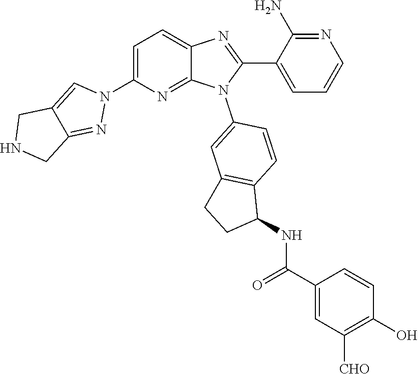 [SMILES: Nc1ncccc1-c1nc2ccc(-n3cc4c(n3)CNC4)nc2n1-c1ccc2c(c1)CC[C@@H]2NC(=O)c1ccc(O)c(C=O)c1] |
| 161 | 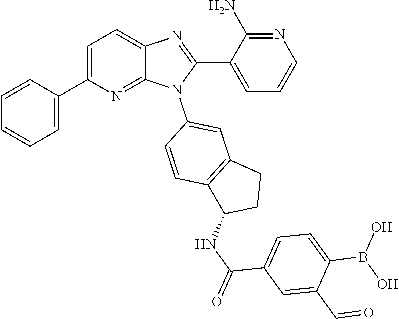 [SMILES: Nc1ncccc1-c1nc2ccc(-c3ccccc3)nc2n1-c1ccc2c(c1)CC[C@@H]2NC(=O)c1ccc(B(O)O)c(C=O)c1] |
| 162 | 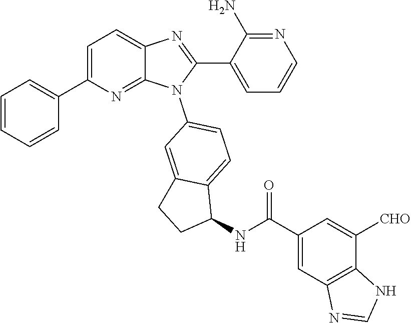 [SMILES: Nc1ncccc1-c1nc2ccc(-c3ccccc3)nc2n1-c1ccc2c(c1)CC[C@@H]2NC(=O)c1cc(C=O)c2[nH]cnc2c1] |
| 163 | 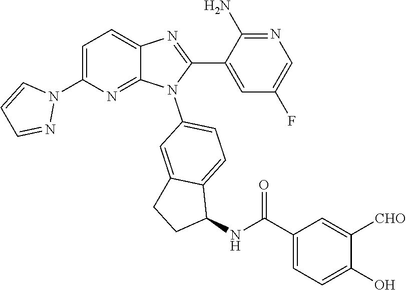 [SMILES: Nc1ncc(F)cc1-c1nc2ccc(-n3cccn3)nc2n1-c1ccc2c(c1)CC[C@@H]2NC(=O)c1ccc(O)c(C=O)c1] |
| 165 | 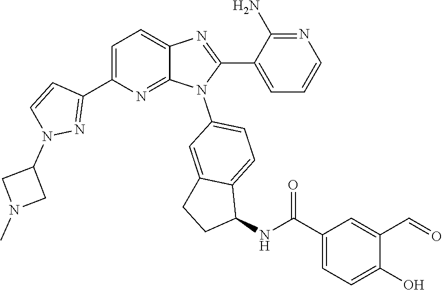 [SMILES: CN1CC(n2ccc(-c3ccc4nc(-c5cccnc5N)n(-c5ccc6c(c5)CC[C@@H]6NC(=O)c5ccc(O)c(C=O)c5)c4n3)n2)C1] |
| 166 | 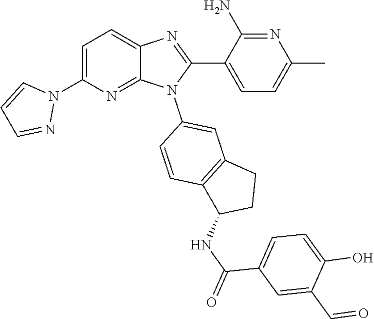 [SMILES: Cc1ccc(-c2nc3ccc(-n4cccn4)nc3n2-c2ccc3c(c2)CC[C@@H]3NC(=O)c2ccc(O)c(C=O)c2)c(N)n1] |
| 167 | 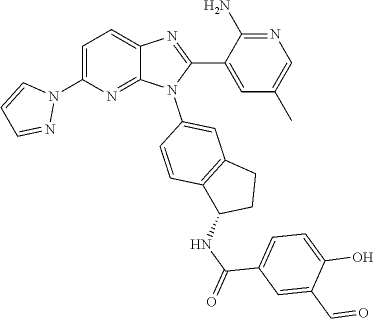 [SMILES: Cc1cnc(N)c(-c2nc3ccc(-n4cccn4)nc3n2-c2ccc3c(c2)CC[C@@H]3NC(=O)c2ccc(O)c(C=O)c2)c1] |
| 168 | 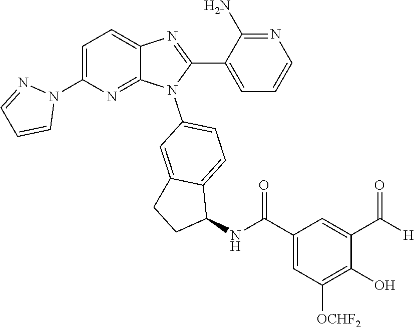 [SMILES: Nc1ncccc1-c1nc2ccc(-n3cccn3)nc2n1-c1ccc2c(c1)CC[C@@H]2NC(=O)c1cc(C=O)c(O)c(OC(F)F)c1] |
| 169 | 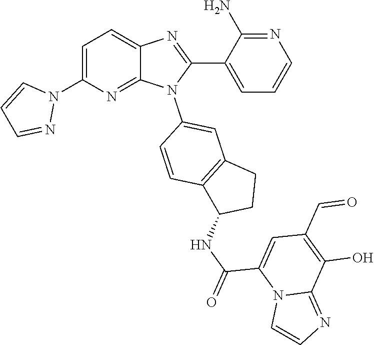 [SMILES: Nc1ncccc1-c1nc2ccc(-n3cccn3)nc2n1-c1ccc2c(c1)CC[C@@H]2NC(=O)c1cc(C=O)c(O)c2nccn12] |
| 170 | 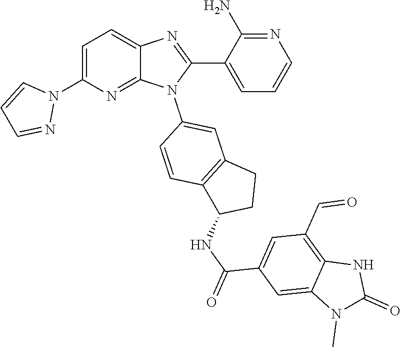 [SMILES: Cn1c(=O)[nH]c2c(C=O)cc(C(=O)N[C@H]3CCc4cc(-n5c(-c6cccnc6N)nc6ccc(-n7cccn7)nc65)ccc43)cc21] |
| 171 | 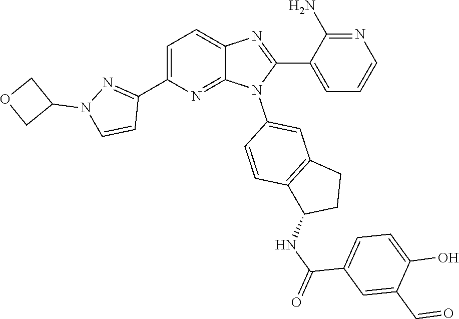 [SMILES: Nc1ncccc1-c1nc2ccc(-c3ccn(C4COC4)n3)nc2n1-c1ccc2c(c1)CC[C@@H]2NC(=O)c1ccc(O)c(C=O)c1] |
| 172 | 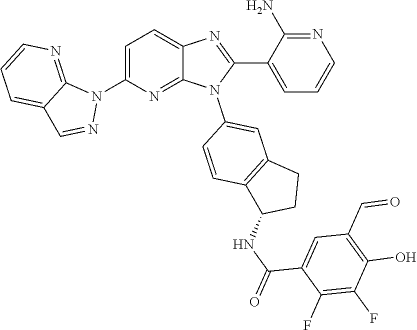 [SMILES: Nc1ncccc1-c1nc2ccc(-n3ncc4cccnc43)nc2n1-c1ccc2c(c1)CC[C@@H]2NC(=O)c1cc(C=O)c(O)c(F)c1F] |
| 173 | 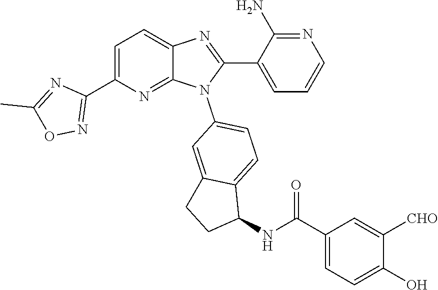 [SMILES: Cc1nc(-c2ccc3nc(-c4cccnc4N)n(-c4ccc5c(c4)CC[C@@H]5NC(=O)c4ccc(O)c(C=O)c4)c3n2)no1] |
| 174 | 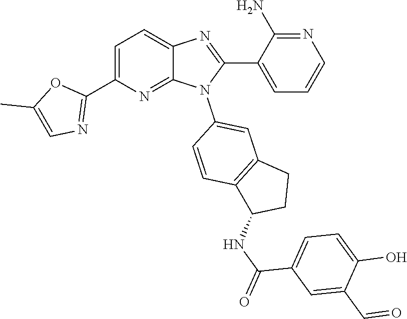 [SMILES: Cc1cnc(-c2ccc3nc(-c4cccnc4N)n(-c4ccc5c(c4)CC[C@@H]5NC(=O)c4ccc(O)c(C=O)c4)c3n2)o1] |
| 175 | 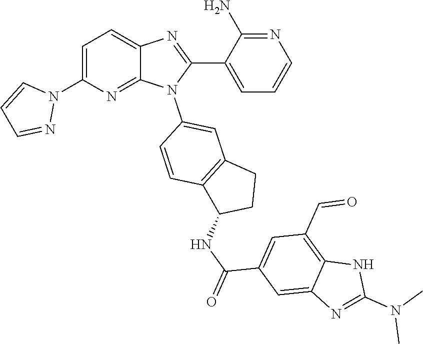 [SMILES: CN(C)c1nc2cc(C(=O)N[C@H]3CCc4cc(-n5c(-c6cccnc6N)nc6ccc(-n7cccn7)nc65)ccc43)cc(C=O)c2[nH]1] |
| 176 | 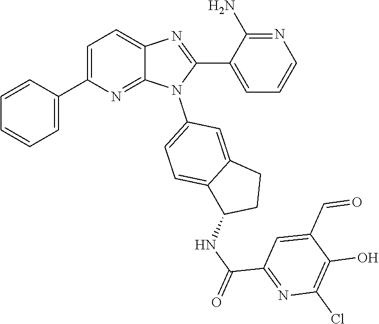 [SMILES: Nc1ncccc1-c1nc2ccc(-c3ccccc3)nc2n1-c1ccc2c(c1)CC[C@@H]2NC(=O)c1cc(C=O)c(O)c(Cl)n1] |
| 177 | 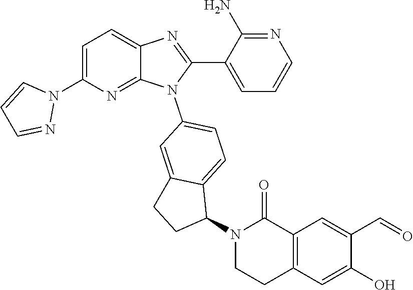 [SMILES: Nc1ncccc1-c1nc2ccc(-n3cccn3)nc2n1-c1ccc2c(c1)CC[C@@H]2N1CCc2cc(O)c(C=O)cc2C1=O] |
| 178 | 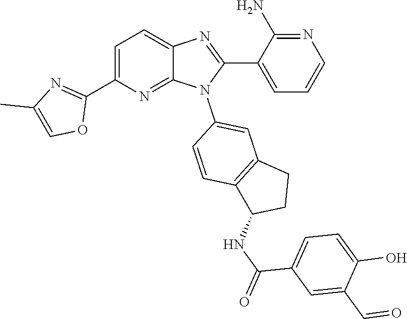 [SMILES: Cc1coc(-c2ccc3nc(-c4cccnc4N)n(-c4ccc5c(c4)CC[C@@H]5NC(=O)c4ccc(O)c(C=O)c4)c3n2)n1] |
| 179 | 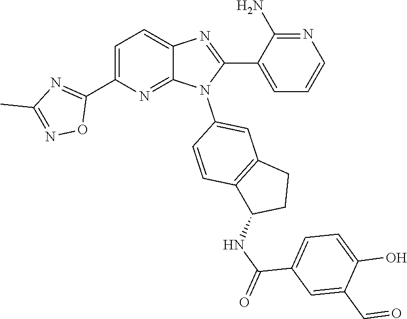 [SMILES: Cc1noc(-c2ccc3nc(-c4cccnc4N)n(-c4ccc5c(c4)CC[C@@H]5NC(=O)c4ccc(O)c(C=O)c4)c3n2)n1] |
| 180 | 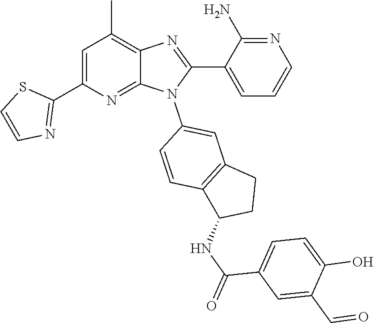 [SMILES: Cc1cc(-c2nccs2)nc2c1nc(-c1cccnc1N)n2-c1ccc2c(c1)CC[C@@H]2NC(=O)c1ccc(O)c(C=O)c1] |
| 181 | 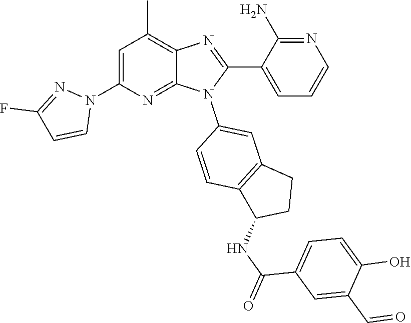 [SMILES: Cc1cc(-n2ccc(F)n2)nc2c1nc(-c1cccnc1N)n2-c1ccc2c(c1)CC[C@@H]2NC(=O)c1ccc(O)c(C=O)c1] |
| 182 | 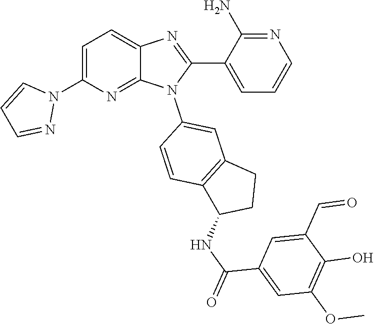 [SMILES: COc1cc(C(=O)N[C@H]2CCc3cc(-n4c(-c5cccnc5N)nc5ccc(-n6cccn6)nc54)ccc32)cc(C=O)c1O] |
| 183 | 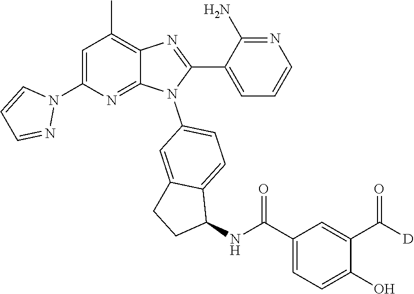 [SMILES: [2H]C(=O)c1cc(C(=O)N[C@H]2CCc3cc(-n4c(-c5cccnc5N)nc5c(C)cc(-n6cccn6)nc54)ccc32)ccc1O] |
| 184 | 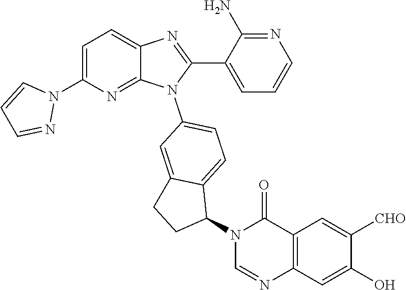 [SMILES: Nc1ncccc1-c1nc2ccc(-n3cccn3)nc2n1-c1ccc2c(c1)CC[C@@H]2n1cnc2cc(O)c(C=O)cc2c1=O] |
| 185 | 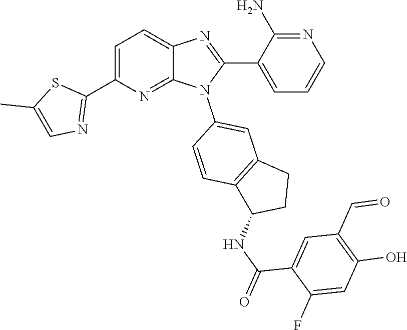 [SMILES: Cc1cnc(-c2ccc3nc(-c4cccnc4N)n(-c4ccc5c(c4)CC[C@@H]5NC(=O)c4cc(C=O)c(O)cc4F)c3n2)s1] |
| 186 | 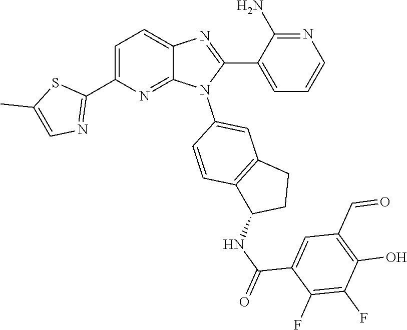 [SMILES: Cc1cnc(-c2ccc3nc(-c4cccnc4N)n(-c4ccc5c(c4)CC[C@@H]5NC(=O)c4cc(C=O)c(O)c(F)c4F)c3n2)s1] |
| 188 | 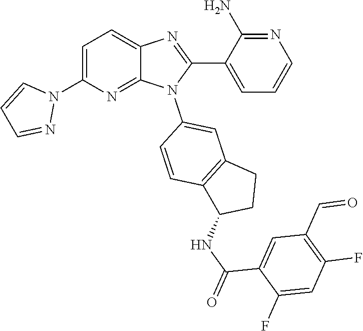 [SMILES: Nc1ncccc1-c1nc2ccc(-n3cccn3)nc2n1-c1ccc2c(c1)CC[C@@H]2NC(=O)c1cc(C=O)c(F)cc1F] |
| 189 | 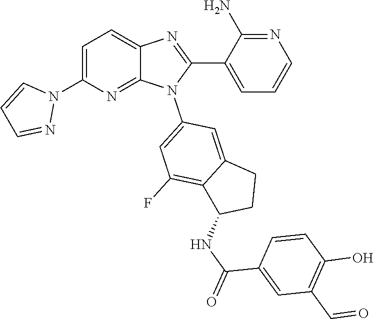 [SMILES: Nc1ncccc1-c1nc2ccc(-n3cccn3)nc2n1-c1cc(F)c2c(c1)CC[C@@H]2NC(=O)c1ccc(O)c(C=O)c1] |
| 190 | 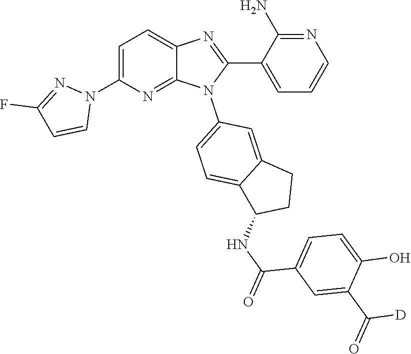 [SMILES: [2H]C(=O)c1cc(C(=O)N[C@H]2CCc3cc(-n4c(-c5cccnc5N)nc5ccc(-n6ccc(F)n6)nc54)ccc32)ccc1O] |
| 191 | 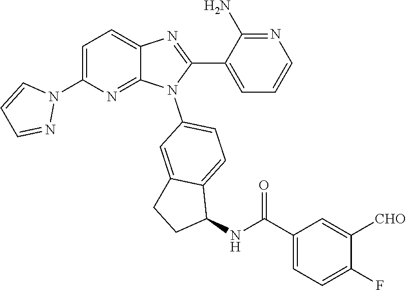 [SMILES: Nc1ncccc1-c1nc2ccc(-n3cccn3)nc2n1-c1ccc2c(c1)CC[C@@H]2NC(=O)c1ccc(F)c(C=O)c1] |
| 192 | 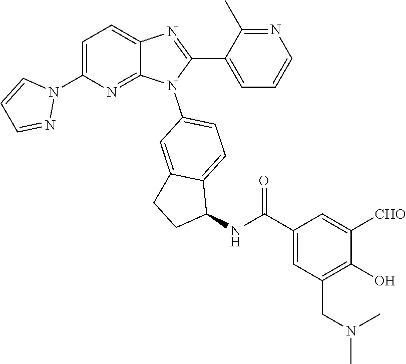 [SMILES: Cc1ncccc1-c1nc2ccc(-n3cccn3)nc2n1-c1ccc2c(c1)CC[C@@H]2NC(=O)c1cc(C=O)c(O)c(CN(C)C)c1] |
| 193 | 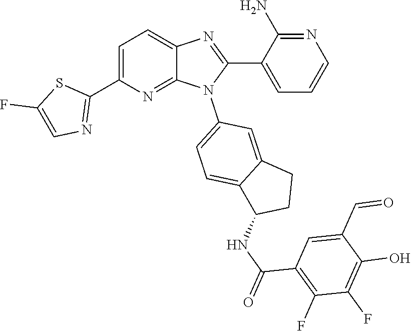 [SMILES: Nc1ncccc1-c1nc2ccc(-c3ncc(F)s3)nc2n1-c1ccc2c(c1)CC[C@@H]2NC(=O)c1cc(C=O)c(O)c(F)c1F] |
| 194 | 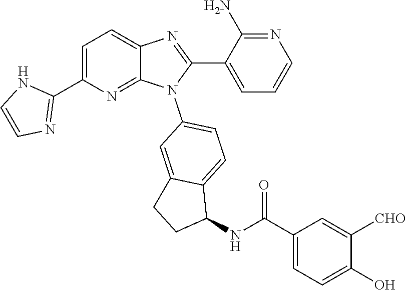 [SMILES: Nc1ncccc1-c1nc2ccc(-c3ncc[nH]3)nc2n1-c1ccc2c(c1)CC[C@@H]2NC(=O)c1ccc(O)c(C=O)c1] |
| 195 | 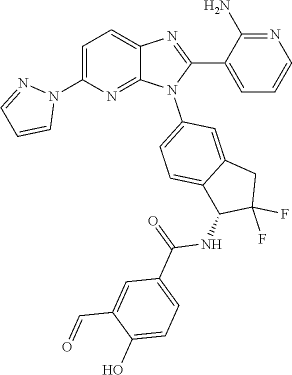 [SMILES: Nc1ncccc1-c1nc2ccc(-n3cccn3)nc2n1-c1ccc2c(c1)CC(F)(F)[C@@H]2NC(=O)c1ccc(O)c(C=O)c1] |
| 196 | 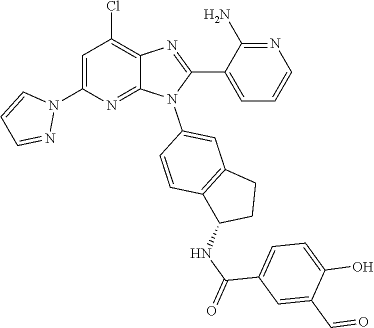 [SMILES: Nc1ncccc1-c1nc2c(Cl)cc(-n3cccn3)nc2n1-c1ccc2c(c1)CC[C@@H]2NC(=O)c1ccc(O)c(C=O)c1] |
| 197 | 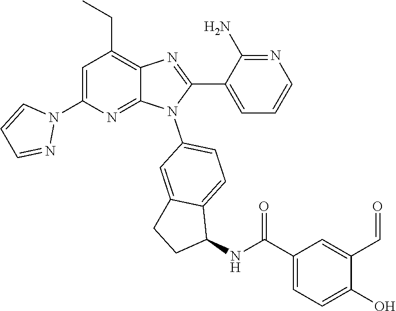 [SMILES: CCc1cc(-n2cccn2)nc2c1nc(-c1cccnc1N)n2-c1ccc2c(c1)CC[C@@H]2NC(=O)c1ccc(O)c(C=O)c1] |
| 198 | 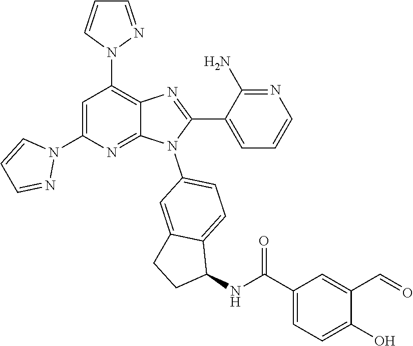 [SMILES: Nc1ncccc1-c1nc2c(-n3cccn3)cc(-n3cccn3)nc2n1-c1ccc2c(c1)CC[C@@H]2NC(=O)c1ccc(O)c(C=O)c1] |
| 199 | 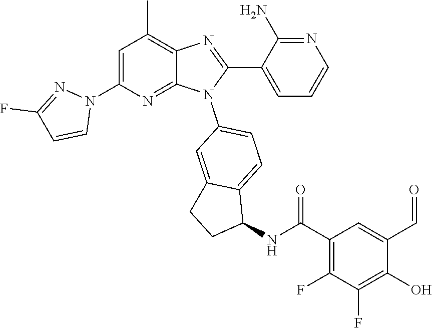 [SMILES: Cc1cc(-n2ccc(F)n2)nc2c1nc(-c1cccnc1N)n2-c1ccc2c(c1)CC[C@@H]2NC(=O)c1cc(C=O)c(O)c(F)c1F] |
| 200 | 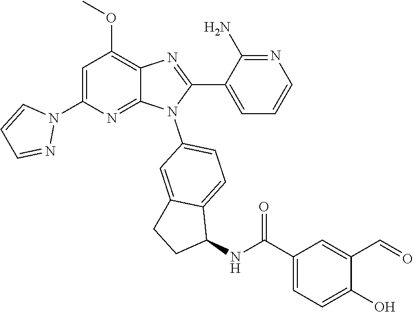 [SMILES: COc1cc(-n2cccn2)nc2c1nc(-c1cccnc1N)n2-c1ccc2c(c1)CC[C@@H]2NC(=O)c1ccc(O)c(C=O)c1] |
| 201 | 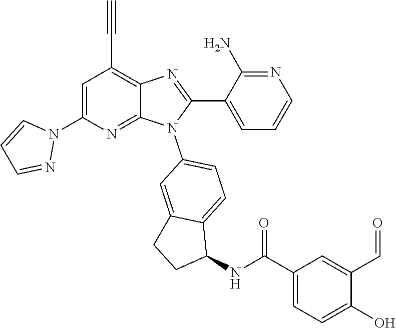 [SMILES: C#Cc1cc(-n2cccn2)nc2c1nc(-c1cccnc1N)n2-c1ccc2c(c1)CC[C@@H]2NC(=O)c1ccc(O)c(C=O)c1] |
| 202 | 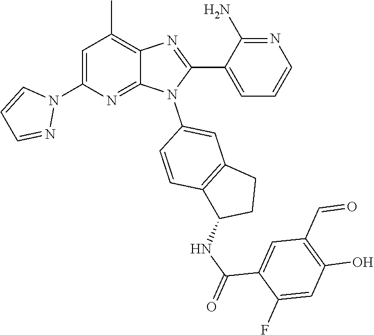 [SMILES: Cc1cc(-n2cccn2)nc2c1nc(-c1cccnc1N)n2-c1ccc2c(c1)CC[C@@H]2NC(=O)c1cc(C=O)c(O)cc1F] |
| 203 | 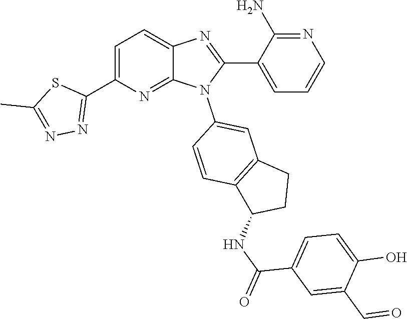 [SMILES: Cc1nnc(-c2ccc3nc(-c4cccnc4N)n(-c4ccc5c(c4)CC[C@@H]5NC(=O)c4ccc(O)c(C=O)c4)c3n2)s1] |
| 204 | 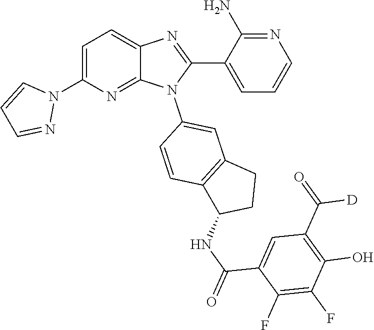 [SMILES: [2H]C(=O)c1cc(C(=O)N[C@H]2CCc3cc(-n4c(-c5cccnc5N)nc5ccc(-n6cccn6)nc54)ccc32)c(F)c(F)c1O] |
| 205 | 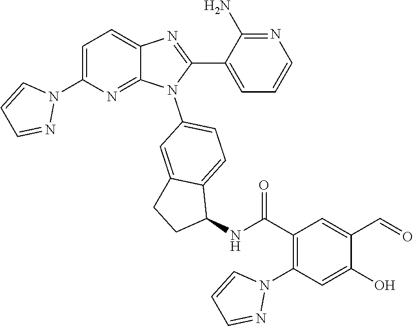 [SMILES: Nc1ncccc1-c1nc2ccc(-n3cccn3)nc2n1-c1ccc2c(c1)CC[C@@H]2NC(=O)c1cc(C=O)c(O)cc1-n1cccn1] |
| 206 | 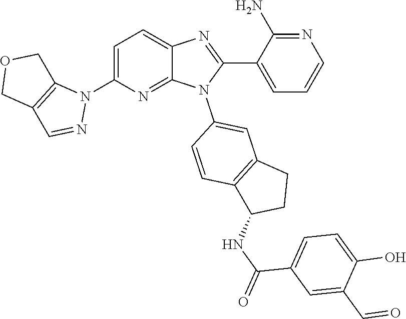 [SMILES: Nc1ncccc1-c1nc2ccc(-n3ncc4c3COC4)nc2n1-c1ccc2c(c1)CC[C@@H]2NC(=O)c1ccc(O)c(C=O)c1] |
| 207 | 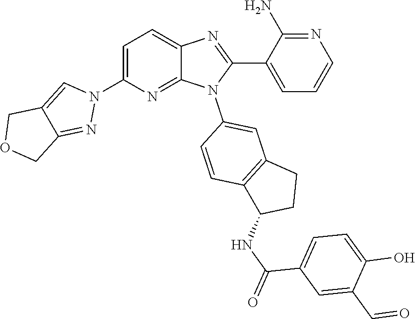 [SMILES: Nc1ncccc1-c1nc2ccc(-n3cc4c(n3)COC4)nc2n1-c1ccc2c(c1)CC[C@@H]2NC(=O)c1ccc(O)c(C=O)c1] |
| 208 | 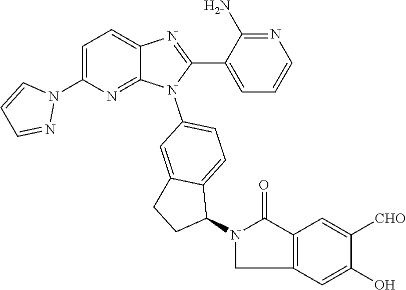 [SMILES: Nc1ncccc1-c1nc2ccc(-n3cccn3)nc2n1-c1ccc2c(c1)CC[C@@H]2N1Cc2cc(O)c(C=O)cc2C1=O] |
| 209 | 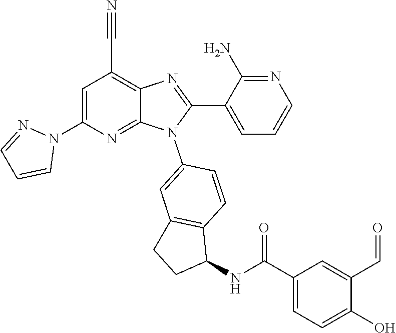 [SMILES: N#Cc1cc(-n2cccn2)nc2c1nc(-c1cccnc1N)n2-c1ccc2c(c1)CC[C@@H]2NC(=O)c1ccc(O)c(C=O)c1] |
| 210 | 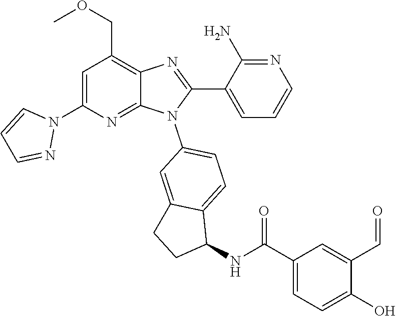 [SMILES: COCc1cc(-n2cccn2)nc2c1nc(-c1cccnc1N)n2-c1ccc2c(c1)CC[C@@H]2NC(=O)c1ccc(O)c(C=O)c1] |
| 211 | 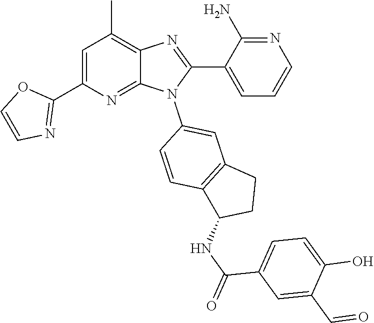 [SMILES: Cc1cc(-c2ncco2)nc2c1nc(-c1cccnc1N)n2-c1ccc2c(c1)CC[C@@H]2NC(=O)c1ccc(O)c(C=O)c1] |
| 212 | 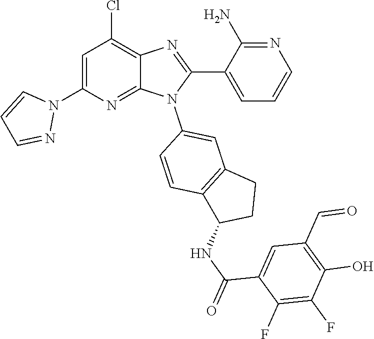 [SMILES: Nc1ncccc1-c1nc2c(Cl)cc(-n3cccn3)nc2n1-c1ccc2c(c1)CC[C@@H]2NC(=O)c1cc(C=O)c(O)c(F)c1F] |
| 213 | 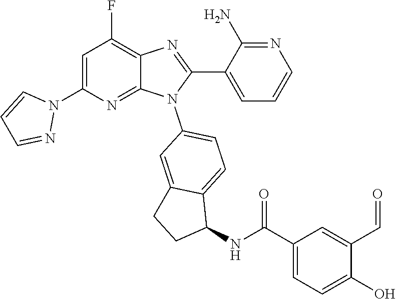 [SMILES: Nc1ncccc1-c1nc2c(F)cc(-n3cccn3)nc2n1-c1ccc2c(c1)CC[C@@H]2NC(=O)c1ccc(O)c(C=O)c1] |
| 214 | 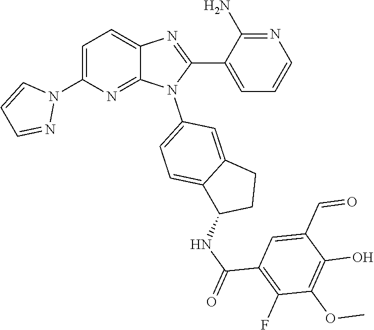 [SMILES: COc1c(O)c(C=O)cc(C(=O)N[C@H]2CCc3cc(-n4c(-c5cccnc5N)nc5ccc(-n6cccn6)nc54)ccc32)c1F] |
| 215 | 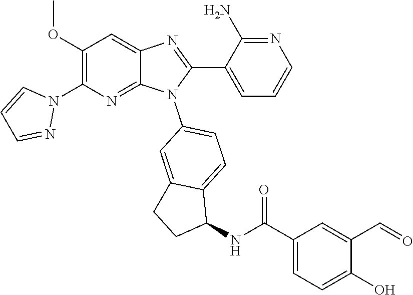 [SMILES: COc1cc2nc(-c3cccnc3N)n(-c3ccc4c(c3)CC[C@@H]4NC(=O)c3ccc(O)c(C=O)c3)c2nc1-n1cccn1] |
| 216 | 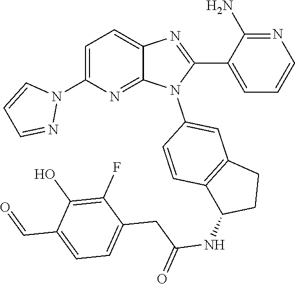 [SMILES: Nc1ncccc1-c1nc2ccc(-n3cccn3)nc2n1-c1ccc2c(c1)CC[C@@H]2NC(=O)Cc1ccc(C=O)c(O)c1F] |
| 217 | 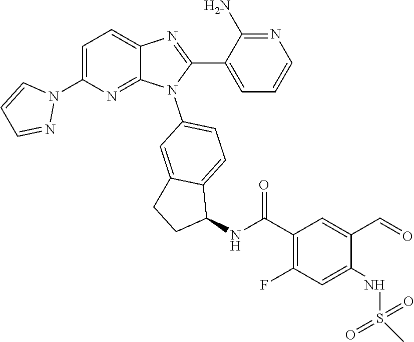 [SMILES: CS(=O)(=O)Nc1cc(F)c(C(=O)N[C@H]2CCc3cc(-n4c(-c5cccnc5N)nc5ccc(-n6cccn6)nc54)ccc32)cc1C=O] |
| 218 | 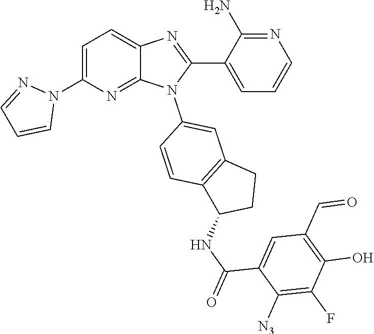 [SMILES: [N-]=[N+]=Nc1c(C(=O)N[C@H]2CCc3cc(-n4c(-c5cccnc5N)nc5ccc(-n6cccn6)nc54)ccc32)cc(C=O)c(O)c1F] |
| 219 | 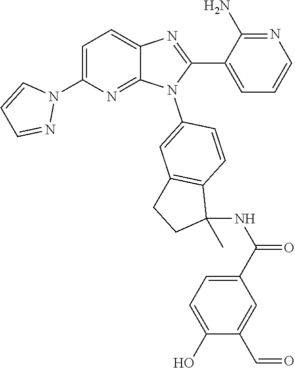 [SMILES: CC1(NC(=O)c2ccc(O)c(C=O)c2)CCc2cc(-n3c(-c4cccnc4N)nc4ccc(-n5cccn5)nc43)ccc21] |
| 220 | 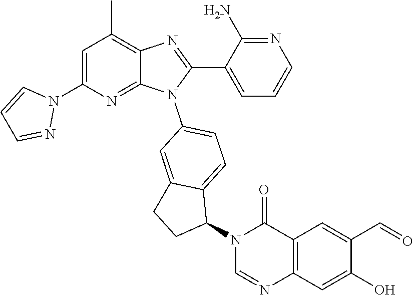 [SMILES: Cc1cc(-n2cccn2)nc2c1nc(-c1cccnc1N)n2-c1ccc2c(c1)CC[C@@H]2n1cnc2cc(O)c(C=O)cc2c1=O] |
| 221 | 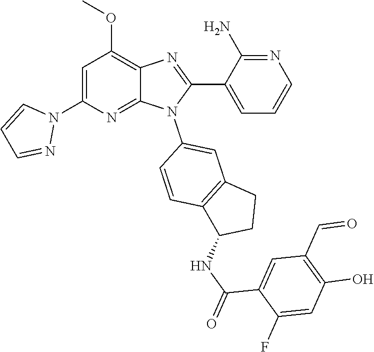 [SMILES: COc1cc(-n2cccn2)nc2c1nc(-c1cccnc1N)n2-c1ccc2c(c1)CC[C@@H]2NC(=O)c1cc(C=O)c(O)cc1F] |
| 222 | 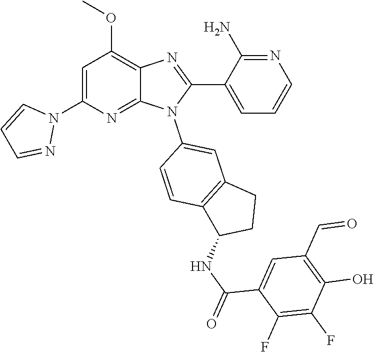 [SMILES: COc1cc(-n2cccn2)nc2c1nc(-c1cccnc1N)n2-c1ccc2c(c1)CC[C@@H]2NC(=O)c1cc(C=O)c(O)c(F)c1F] |
| 223 | 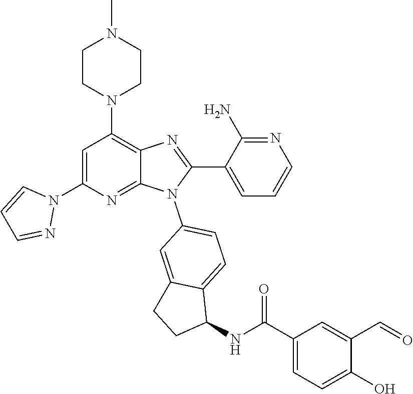 [SMILES: CN1CCN(c2cc(-n3cccn3)nc3c2nc(-c2cccnc2N)n3-c2ccc3c(c2)CC[C@@H]3NC(=O)c2ccc(O)c(C=O)c2)CC1] |
| 224 | 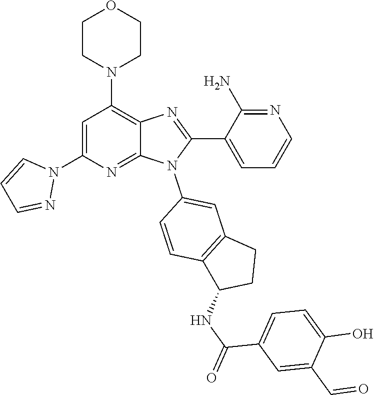 [SMILES: Nc1ncccc1-c1nc2c(N3CCOCC3)cc(-n3cccn3)nc2n1-c1ccc2c(c1)CC[C@@H]2NC(=O)c1ccc(O)c(C=O)c1] |
| 225 | 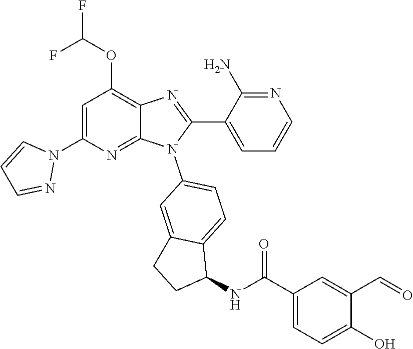 [SMILES: Nc1ncccc1-c1nc2c(OC(F)F)cc(-n3cccn3)nc2n1-c1ccc2c(c1)CC[C@@H]2NC(=O)c1ccc(O)c(C=O)c1] |
| 226 | 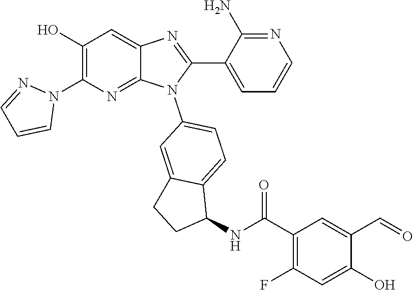 [SMILES: Nc1ncccc1-c1nc2cc(O)c(-n3cccn3)nc2n1-c1ccc2c(c1)CC[C@@H]2NC(=O)c1cc(C=O)c(O)cc1F] |
| 227 | 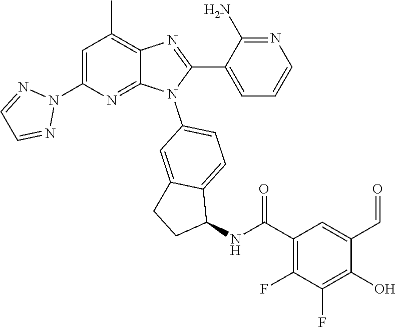 [SMILES: Cc1cc(-n2nccn2)nc2c1nc(-c1cccnc1N)n2-c1ccc2c(c1)CC[C@@H]2NC(=O)c1cc(C=O)c(O)c(F)c1F] |
| 228 | 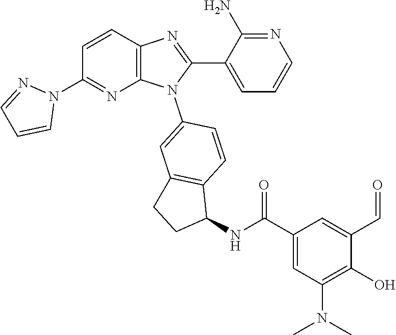 [SMILES: CN(C)c1cc(C(=O)N[C@H]2CCc3cc(-n4c(-c5cccnc5N)nc5ccc(-n6cccn6)nc54)ccc32)cc(C=O)c1O] |
| 229 | 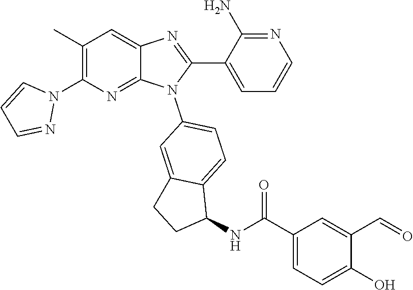 [SMILES: Cc1cc2nc(-c3cccnc3N)n(-c3ccc4c(c3)CC[C@@H]4NC(=O)c3ccc(O)c(C=O)c3)c2nc1-n1cccn1] |
| 230 | 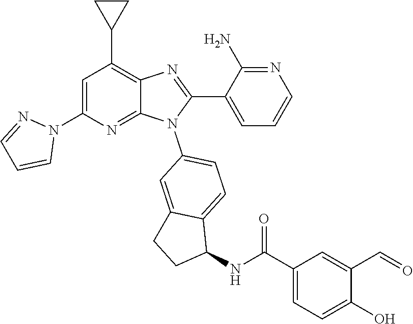 [SMILES: Nc1ncccc1-c1nc2c(C3CC3)cc(-n3cccn3)nc2n1-c1ccc2c(c1)CC[C@@H]2NC(=O)c1ccc(O)c(C=O)c1] |
| 231 | 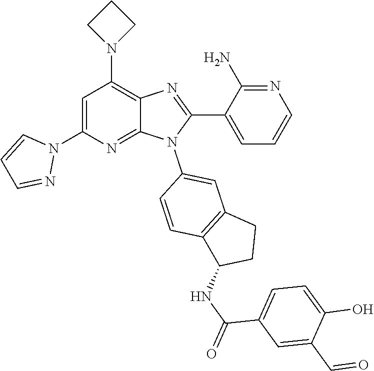 [SMILES: Nc1ncccc1-c1nc2c(N3CCC3)cc(-n3cccn3)nc2n1-c1ccc2c(c1)CC[C@@H]2NC(=O)c1ccc(O)c(C=O)c1] |
| 232 | 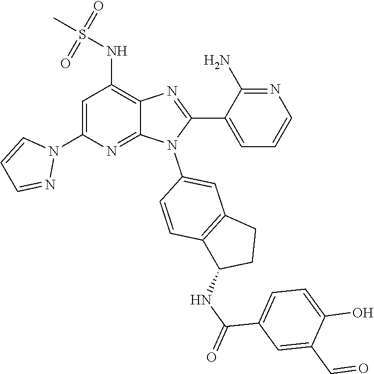 [SMILES: CS(=O)(=O)Nc1cc(-n2cccn2)nc2c1nc(-c1cccnc1N)n2-c1ccc2c(c1)CC[C@@H]2NC(=O)c1ccc(O)c(C=O)c1] |
| 233 | 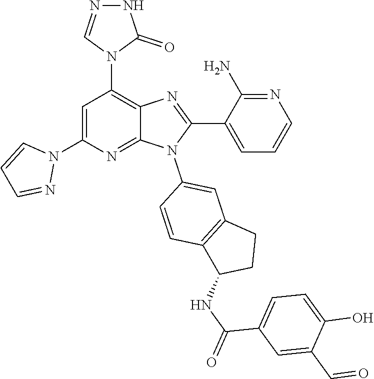 [SMILES: Nc1ncccc1-c1nc2c(-n3cn[nH]c3=O)cc(-n3cccn3)nc2n1-c1ccc2c(c1)CC[C@@H]2NC(=O)c1ccc(O)c(C=O)c1] |
| 234 | 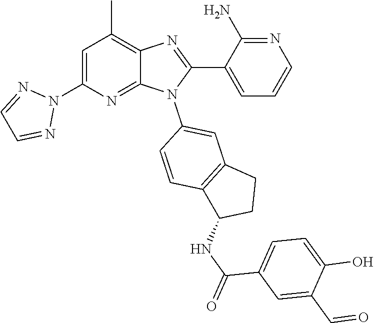 [SMILES: Cc1cc(-n2nccn2)nc2c1nc(-c1cccnc1N)n2-c1ccc2c(c1)CC[C@@H]2NC(=O)c1ccc(O)c(C=O)c1] |
| 235 | 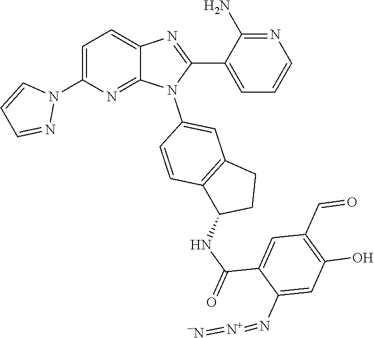 |
| 236 | 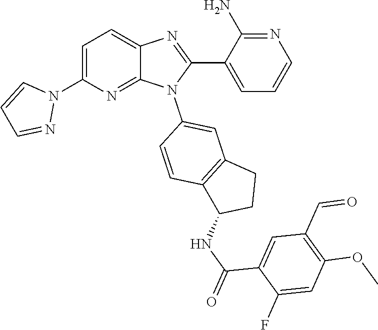 [SMILES: COc1cc(F)c(C(=O)N[C@H]2CCc3cc(-n4c(-c5cccnc5N)nc5ccc(-n6cccn6)nc54)ccc32)cc1C=O] |
| 237 | 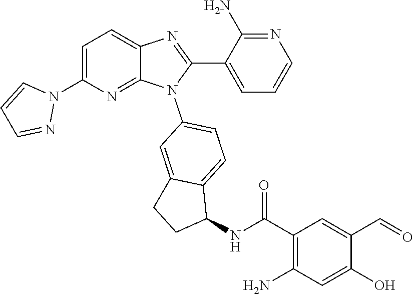 [SMILES: Nc1cc(O)c(C=O)cc1C(=O)N[C@H]1CCc2cc(-n3c(-c4cccnc4N)nc4ccc(-n5cccn5)nc43)ccc21] |
| 238 | 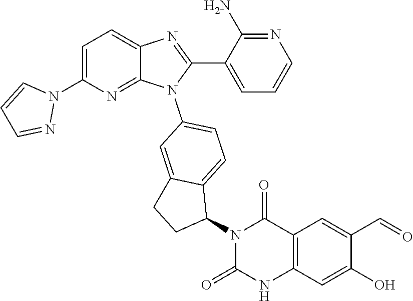 [SMILES: Nc1ncccc1-c1nc2ccc(-n3cccn3)nc2n1-c1ccc2c(c1)CC[C@@H]2n1c(=O)[nH]c2cc(O)c(C=O)cc2c1=O] |
| 239 | 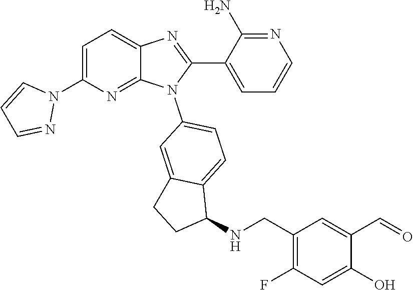 [SMILES: Nc1ncccc1-c1nc2ccc(-n3cccn3)nc2n1-c1ccc2c(c1)CC[C@@H]2NCc1cc(C=O)c(O)cc1F] |
| 240 | 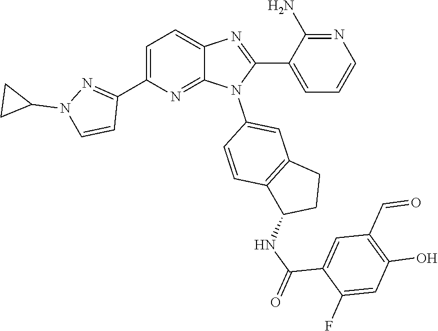 [SMILES: Nc1ncccc1-c1nc2ccc(-c3ccn(C4CC4)n3)nc2n1-c1ccc2c(c1)CC[C@@H]2NC(=O)c1cc(C=O)c(O)cc1F] |
| 241 | 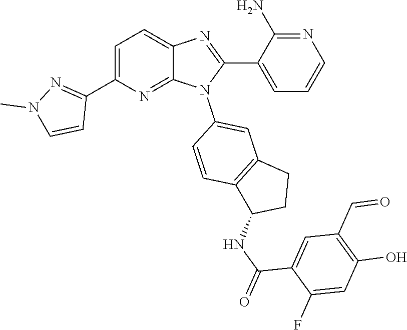 [SMILES: Cn1ccc(-c2ccc3nc(-c4cccnc4N)n(-c4ccc5c(c4)CC[C@@H]5NC(=O)c4cc(C=O)c(O)cc4F)c3n2)n1] |
| 242 | 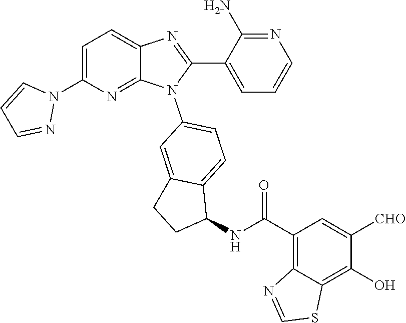 [SMILES: Nc1ncccc1-c1nc2ccc(-n3cccn3)nc2n1-c1ccc2c(c1)CC[C@@H]2NC(=O)c1cc(C=O)c(O)c2scnc12] |
| 243 | 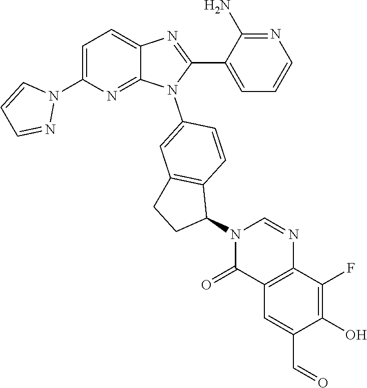 [SMILES: Nc1ncccc1-c1nc2ccc(-n3cccn3)nc2n1-c1ccc2c(c1)CC[C@@H]2n1cnc2c(F)c(O)c(C=O)cc2c1=O] |
| 244 | 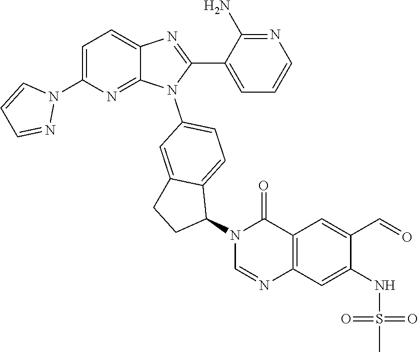 [SMILES: CS(=O)(=O)Nc1cc2ncn([C@H]3CCc4cc(-n5c(-c6cccnc6N)nc6ccc(-n7cccn7)nc65)ccc43)c(=O)c2cc1C=O] |
| 245 | 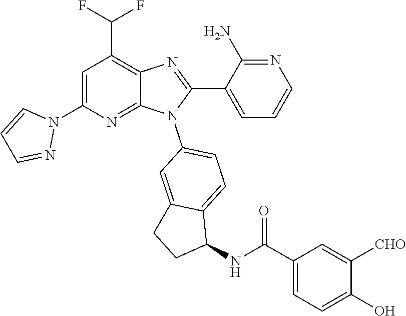 [SMILES: Nc1ncccc1-c1nc2c(C(F)F)cc(-n3cccn3)nc2n1-c1ccc2c(c1)CC[C@@H]2NC(=O)c1ccc(O)c(C=O)c1] |
| 246 | 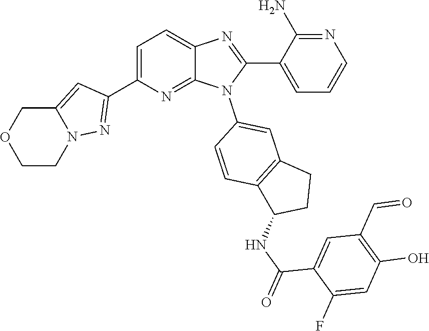 [SMILES: Nc1ncccc1-c1nc2ccc(-c3cc4n(n3)CCOC4)nc2n1-c1ccc2c(c1)CC[C@@H]2NC(=O)c1cc(C=O)c(O)cc1F] |
| 247 | 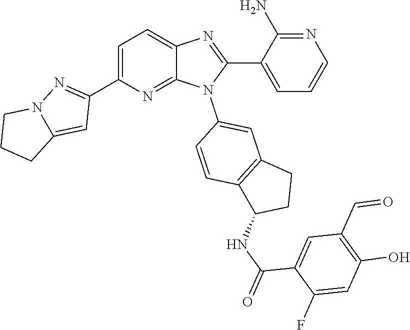 [SMILES: Nc1ncccc1-c1nc2ccc(-c3cc4n(n3)CCC4)nc2n1-c1ccc2c(c1)CC[C@@H]2NC(=O)c1cc(C=O)c(O)cc1F] |
| 248 | 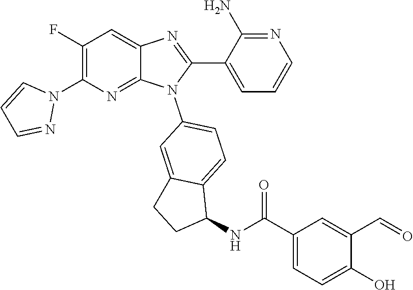 [SMILES: Nc1ncccc1-c1nc2cc(F)c(-n3cccn3)nc2n1-c1ccc2c(c1)CC[C@@H]2NC(=O)c1ccc(O)c(C=O)c1] |
| 249 | 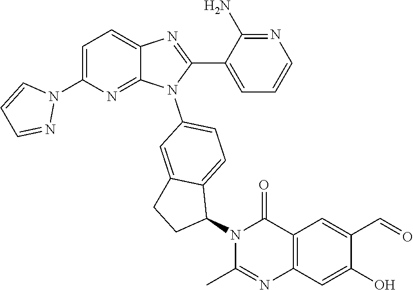 [SMILES: Cc1nc2cc(O)c(C=O)cc2c(=O)n1[C@H]1CCc2cc(-n3c(-c4cccnc4N)nc4ccc(-n5cccn5)nc43)ccc21] |
| 250 | 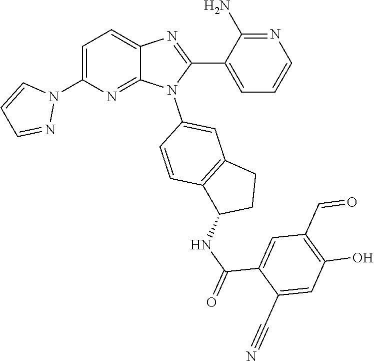 [SMILES: N#Cc1cc(O)c(C=O)cc1C(=O)N[C@H]1CCc2cc(-n3c(-c4cccnc4N)nc4ccc(-n5cccn5)nc43)ccc21] |
| 251 | 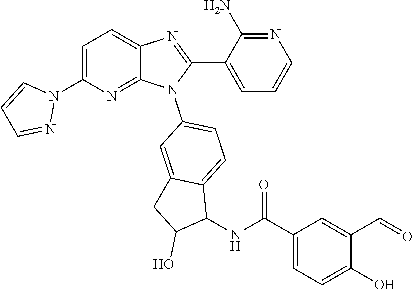 [SMILES: Nc1ncccc1-c1nc2ccc(-n3cccn3)nc2n1-c1ccc2c(c1)CC(O)C2NC(=O)c1ccc(O)c(C=O)c1] |
| 252 | 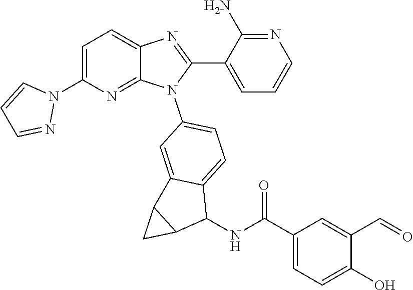 [SMILES: Nc1ncccc1-c1nc2ccc(-n3cccn3)nc2n1-c1ccc2c(c1)C1CC1C2NC(=O)c1ccc(O)c(C=O)c1] |
| 253 | 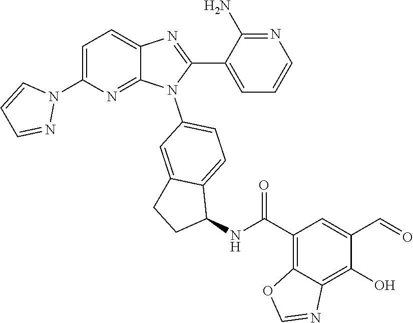 [SMILES: Nc1ncccc1-c1nc2ccc(-n3cccn3)nc2n1-c1ccc2c(c1)CC[C@@H]2NC(=O)c1cc(C=O)c(O)c2ncoc12] |
| 254 | 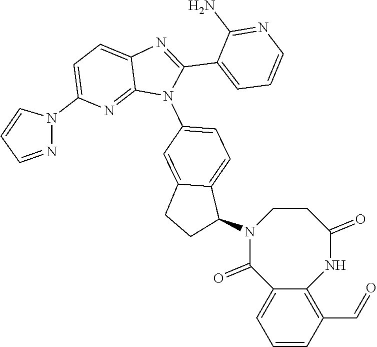 [SMILES: Nc1ncccc1-c1nc2ccc(-n3cccn3)nc2n1-c1ccc2c(c1)CC[C@@H]2N1CCC(=O)Nc2c(C=O)cccc2C1=O] |
| 255 | 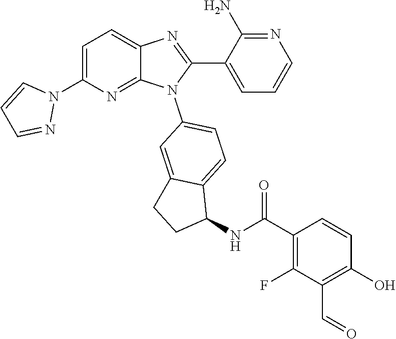 [SMILES: Nc1ncccc1-c1nc2ccc(-n3cccn3)nc2n1-c1ccc2c(c1)CC[C@@H]2NC(=O)c1ccc(O)c(C=O)c1F] |
| 256 | 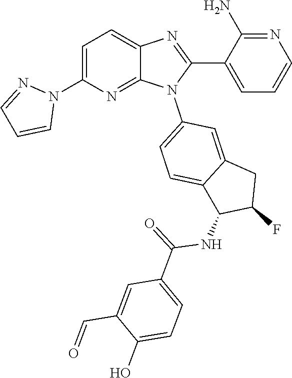 [SMILES: Nc1ncccc1-c1nc2ccc(-n3cccn3)nc2n1-c1ccc2c(c1)C[C@@H](F)[C@@H]2NC(=O)c1ccc(O)c(C=O)c1] |
| 257 | 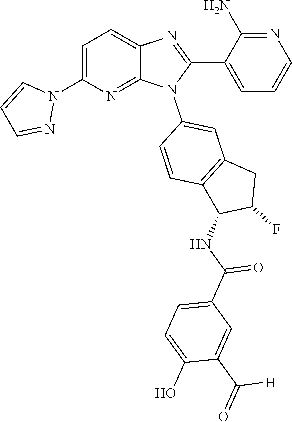 [SMILES: Nc1ncccc1-c1nc2ccc(-n3cccn3)nc2n1-c1ccc2c(c1)C[C@H](F)[C@@H]2NC(=O)c1ccc(O)c(C=O)c1] |
| 258 | 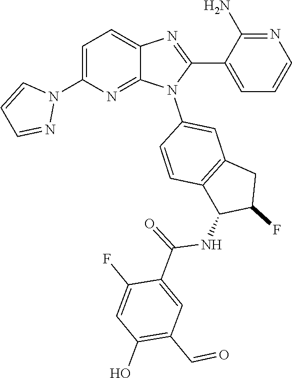 [SMILES: Nc1ncccc1-c1nc2ccc(-n3cccn3)nc2n1-c1ccc2c(c1)C[C@@H](F)[C@@H]2NC(=O)c1cc(C=O)c(O)cc1F] |
| 259 | 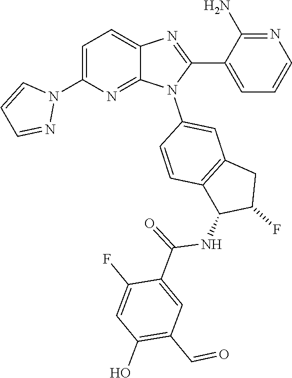 [SMILES: Nc1ncccc1-c1nc2ccc(-n3cccn3)nc2n1-c1ccc2c(c1)C[C@H](F)[C@@H]2NC(=O)c1cc(C=O)c(O)cc1F] |

In certain embodiments, a compound or salt of the disclosure is selected from a compound as described in the Examples herein or a salt thereof.

In certain aspects, the disclosure provides a compound or salt represented by the structure of Formula (A), (A-1), or (I), wherein:
R¹ is selected from hydrogen, halogen, C₁₋₆ alkyl, C₂₋₆ alkenyl, C₂₋₆ alkynyl, C₁₋₆ haloalkyl, C₂₋₆ haloalkenyl, and C₂₋₆ haloalkynyl, for example, R¹ is selected from hydrogen;
A¹ is selected from hydrogen, halogen, C₁₋₆ alkyl, C₂₋₆ alkenyl, C₂₋₆ alkynyl, 5- to 10-membered heterocycle and C₃₋₁₀ carbocycle, wherein C₁₋₆ alkyl, C₂₋₆ alkenyl, C₂₋₆ alkynyl, 5- to 10-membered heterocycle and C₃₋₁₀ carbocycle, are each optionally substituted with one or more substituents independently selected from halogen, —OR¹¹, N(R¹¹)₂, —C(O)R¹¹, —NO₂, —CN, C₁₋₆ alkyl, and C₁₋₆ haloalkyl, for example, A¹ is hydrogen;
A² is selected from hydrogen, halogen, C₁₋₆ alkyl, C₂₋₆ alkenyl, C₂₋₆ alkynyl, 5- to 10-membered heterocycle and C₃₋₁₀ carbocycle, wherein C₁₋₆ alkyl, C₂₋₆ alkenyl, C₂₋₆ alkynyl, 5- to 10-membered heterocycle and C₃₋₁₀ carbocycle, are each optionally substituted with one or more substituents independently selected from halogen, —OR¹¹, N(R¹¹)₂, —C(O)R¹¹, —NO₂, —CN, C₁₋₆ alkyl, and C₁₋₆ haloalkyl, for example, A² is pyrazolyl optionally substituted with halogen, C₁₋₆ alkyl, and C₁₋₆ haloalkyl;
q is selected from 1 and 2, for example, q is 1;
m is selected from 0 and 1, for example, m is 0;
n is selected from 0 and 1, for example, n is 0;
R² is independently selected at each instance from halogen, C₁₋₄ alkyl, and C₁₋₄ haloalkyl; for example, R² is halogen;
R³ is independently selected at each instance from halogen, C₁₋₄ alkyl, and C₁₋₄ haloalkyl; for example, R³ is halogen;
p is selected from 0 and 1, for example, p is 0;
R⁴ is independently selected at each instance from halogen, C₁₋₄ alkyl, and C₁₋₄ haloalkyl; for example, R⁴ is halogen;
L is a represented by -L¹-L²-, wherein L¹ and L² are each independently selected from (a) and (b):
   (a) —O—, —N(R¹⁵)—, —N(R¹⁵)C(O)—, —N(R¹⁵)C(O)O—, —N(R¹⁵)S(O)₂—, —N(R¹⁵)S(O)₂N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
   (b) C₁₋₆ alkylene, C₃₋₈ carbocyclene, and 3- to 8-membered heterocyclene, any of which is optionally substituted with one or more substituents independently selected from halogen, —OR¹⁵, and —CN;
   wherein L² is optionally absent; wherein both of L¹ and L² are not selected from (a), for example, L is -L¹-, and L¹ is selected from —N(R¹¹)—, —N(R¹⁵)C(O)—, and —N(R¹⁵)C(O)O—;
R⁵ is C₃₋₁₂ carbocycle substituted by —C(O)H or —C(O)D, wherein R⁵ is further optionally substituted by one or more substituents independently selected from halogen, —OR¹⁶, —N(R¹⁶)₂, —C(O)R¹⁶, —NO₂, —CN, and C₁₋₆ alkyl optionally substituted with one or more substituents independently selected from halogen, —OR¹⁶, —N(R¹⁶)₂, —C(O)R¹⁶, —NO₂, and —CN, for example, R⁵ is phenyl optionally substituted with halogen, OR¹⁶, C₁₋₆ alkyl, and C₁₋₆ haloalkyl; and
R¹¹, R¹⁵, and R¹⁶ are each independently selected at each occurrence from: hydrogen, C₁₋₄ alkyl, and C₁₋₄ haloalkyl, for example R¹¹, R¹⁵, and R¹⁶ are each hydrogen.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

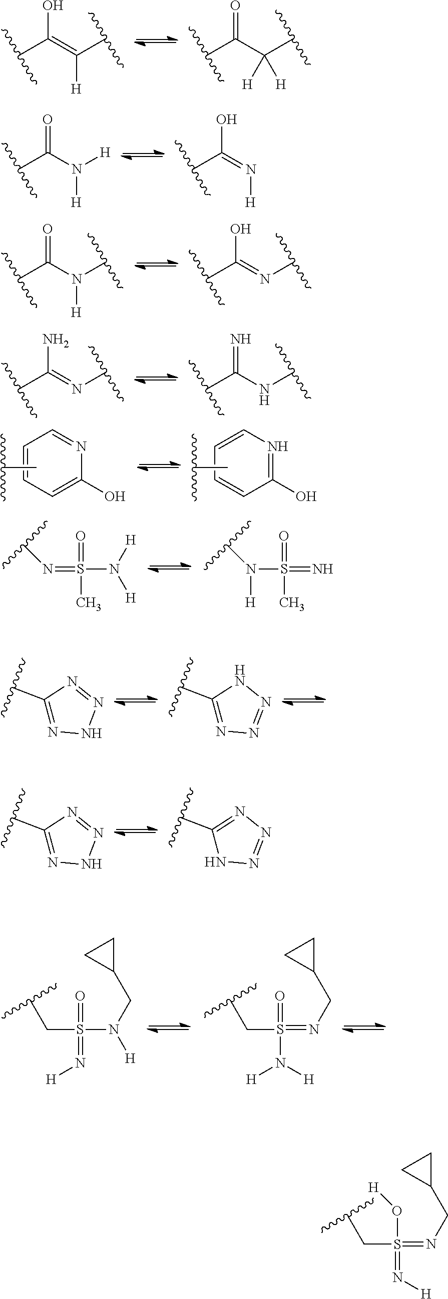

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B) include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B), may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

### Pharmaceutical Formulations

In some aspects, the present disclosure provides a pharmaceutical composition comprising a compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B) and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

A compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B) may be formulated in any suitable pharmaceutical formulation. A pharmaceutical formulation of the present disclosure typically contains an active ingredient (e.g., compound or salt of any one of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B)), and one or more pharmaceutically acceptable excipients or carriers, including but not limited to: inert solid diluents and fillers, diluents, sterile aqueous solution and various organic solvents, permeation enhancers, antioxidants, solubilizers, and adjuvants.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990;
Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000;
Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some aspects, the present disclosure provides a method for treatment, comprising administering to a subject in need thereof an effective amount of a compound or salt of Formula (A), (A-1), (I), (I-A), (II), (II-A), (II-B), (18), (III-A), (III-B), (IV), (IV-A), (IV-B), (V), (V-A), (IX-B), (X), (X-A), (X-B), (XI), (XI-A), or (XI-B). In some aspects, the present disclosure provides a method for treating cancer in a patient in need thereof, comprising administering to the subject an effective amount of a compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B). In some embodiments, the cancer is selected from breast cancer, colorectal cancer, and meningioma. In some embodiments, the cancer is breast cancer. In some embodiments, the cancer is colorectal cancer. In some embodiments, the cancer is meningioma.

In certain embodiments, the present disclosure can be used as a method of inhibiting an AKT1 protein in a subject in need thereof, comprising administering to the subject a compound or salt of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B) or a pharmaceutical composition of Formula (A), (A-1), (I), (I-A), (II), (II-A), (II-B), (III), (III-A), (III-B), (IV), (IV-A), (IV-B), (V), (V-A), (V-B), (VI), (VI-A), (VI-B), (VII), (VII-A), (VII-B), (VIII), (VIII-A), (VIII-B), (IX), (IX-A), (IX-B), (X), (X-A), (X-B), (XI), (XI-A), or (XI-B). In some embodiments, the AKT protein is a mutant AKT1 protein. In some embodiments, the mutant AKT1 protein comprises an E17K mutant. In some embodiments, the administrating modulates the activity of mutant AKT1.

Terremoto Biosciences, Inc., and The Regents Of The University Of California are parties to a joint research agreement executed before Feb. 24, 2023.

### EXAMPLES

The invention now being generally described, it will be more readily understood by reference to the following examples which are included merely for purposes of illustration of certain aspects and embodiments of the present invention and are not intended to limit the invention in any way.

### Chemical Synthesis

The following examples describe illustrate various methods of preparation of compounds described herein. Examples are exemplary and not exhaustive. It is understood that one skilled in the art may be able to synthesize described compounds by similar methods.

### Example 1: 4-(2-((5-(2-(2-aminopyridin-3-yl)-5-phenyl-3H-imidazo[4,5-b]pyridin-3-yl)-2,3-dihydro-1H-inden-1-yl)amino)ethyl)-2-hydroxybenzaldehyde

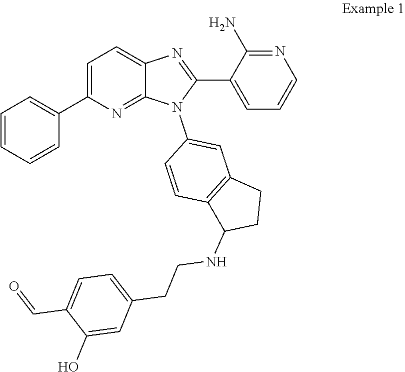 [SMILES: Nc1ncccc1-c1nc2ccc(-c3ccccc3)nc2n1-c1ccc2c(c1)CCC2NCCc1ccc(C=O)c(O)c1]

### Synthetic Route:

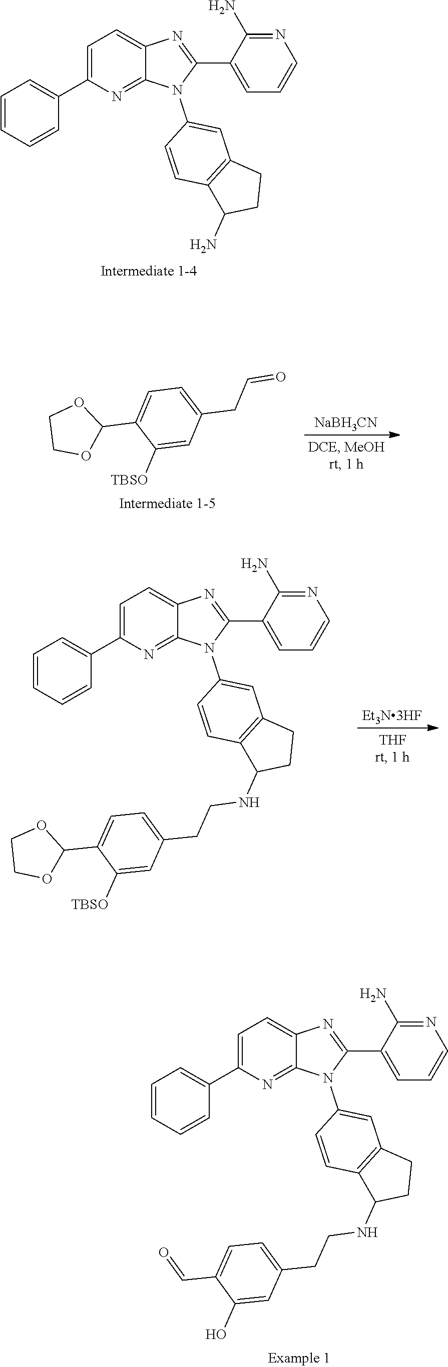 [SMILES: Nc1ncccc1-c1nc2ccc(-c3ccccc3)nc2n1-c1ccc2c(c1)CCC2N ; CC(C)(C)[Si](C)(C)Oc1cc(CC=O)ccc1C1OCCO1 ; "NaBH3CN" ; CC(C)(C)[Si](C)(C)Oc1cc(CCNC2CCc3cc(-n4c(-c5cccnc5N)nc5ccc(-c6ccccc6)nc54)ccc32)ccc1C1OCCO1 ; "Et3N·3HF" ; Nc1ncccc1-c1nc2ccc(-c3ccccc3)nc2n1-c1ccc2c(c1)CCC2NCCc1ccc(C=O)c(O)c1]

### Step 1: Synthesis of 3-(3-{1-[(2-{3-[(tert-butyldimethylsilyl)oxy]-4-(1,3-dioxolan-2-yl)phenyl}ethyl)amino]-2,3-dihydro-1H-inden-5-yl}-5-phenylimidazo[4,5-b]pyridin-2-yl)pyridin-2-amine

3-[3-(1-Amino-2,3-dihydro-1H-inden-5-yl)-5-phenylimidazo[4,5-b]pyridin-2-yl]pyridin-2-amine (Intermediate 1-4) (80 mg, 0.19 mmol, 1 equiv) and 2-{3-[(tert-butyldimethylsilyl)oxy]-4-(1,3-dioxolan-2-yl)phenyl}acetaldehyde (Intermediate 1-5) (124 mg, 0.38 mmol, 2 equiv) were dissolved in 1,2-dichloroethane (7 mL) and methanol (7 mL). The mixture was stirred at room temperature for 30 minutes, followed by addition of sodium cyanoborohydride (36 mg, 0.58 mmol, 3 equiv). The reaction mixture was stirred for an additional 1 h at room temperature. The reaction was quenched with water (20 mL). The resulting mixture was extracted with ethyl acetate (3×20 mL) and dried over anhydrous sodium sulfate, filtered and concentrated in vacuo. The residue was purified by reverse-phase flash chromatography on C18 silica gel using a 20-60% gradient of acetonitrile in water (+0.05% TFA) to afford 3-(3-{1-[(2-{3-[(tert-butyldimethylsilyl)oxy]-4-(1,3-dioxolan-2-yl)phenyl}ethyl)amino]-2,3-dihydro-1H-inden-5-yl}-5-phenylimidazo[4,5-b]pyridin-2-yl)pyridin-2-amine (45 mg, 32%) as a yellow oil. MS (ESI) calculated for C₄₃H₄₈N₆O₃Si:
724.36, found 725.20 [M+H]+.

### Step 2: Synthesis of 4-(2-((5-(2-(2-aminopyridin-3-yl)-5-phenyl-3H-imidazo[4,5-b]pyridin-3-yl)-2,3-dihydro-1H-inden-1-yl)amino)ethyl)-2-hydroxybenzaldehyde (Example 1)

3-(3-{1-[(2-{3-[(tert-butyldimethylsilyl)oxy]-4-(1,3-dioxolan-2-yl)phenyl}ethyl)amino]-2,3-dihydro-1H-inden-5-yl}-5-phenylimidazo[4,5-b]pyridin-2-yl)pyridin-2-amine (70 mg, 0.097 mmol, 1 equiv) was dissolved in tetrahydrofuran (1 mL). Et₃N·3HF (17 mg) was added and the mixture was stirred for 1 h at room temperature. The reaction mixture was concentrated under reduced pressure and purified by preparative HPLC on a Sunfire Prep C18 OBD Column (19×250 mm, 10 μm) using a 15 to 33% gradient of acetonitrile in water (+0.05% TFA) to afford 4-(2-((5-(2-(2-aminopyridin-3-yl)-5-phenyl-3H-imidazo[4,5-b]pyridin-3-yl)-2,3-dihydro-1H-inden-1-yl)amino)ethyl)-2-hydroxybenzaldehyde (Example 1) (4 mg, 3%) as a light yellow solid. MS (ESI) calculated for C₃₅H₃₀N₆O₂: 566.24 m/z, found 567.25 [M+H]⁺. ¹H NMR (400 MHz, DMSO-d₆) δ (ppm): 10.16-10.20 (m, 1H), 8.26-8.29 (m, 1H), 7.98-8.02 (m, 4H), 7.66-7.73 (m, 2H), 7.30-7.55 (m, 6H), 6.91-6.95 (m, 2H), 6.44-6.46 (m, 1H), 4.89-4.90 (m, 1H), 3.26-3.28 (m, 2H), 3.12-3.13 (m, 1H), 2.95-2.97 (m, 3H), 2.54-2.57 (m, 1H), 2.25-2.26 (m, 1H).

### Intermediate 1-1: N-{5-[2-(2-aminopyridin-3-yl)-5-phenylimidazo[4,5-b]pyridin-3-yl]-2,3-dihydro-1H-inden-1-yl}acetamide

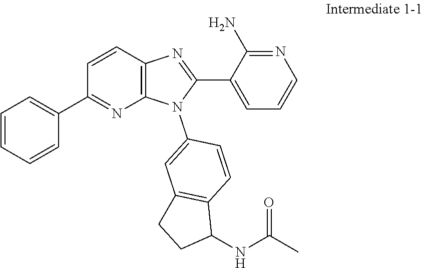 [SMILES: CC(=O)NC1CCc2cc(-n3c(-c4cccnc4N)nc4ccc(-c5ccccc5)nc43)ccc21]

### Intermediate 1-2: (R)-3-(3-(1-amino-2,3-dihydro-1H-inden-5-yl)-5-phenyl-3H-imidazo[4,5-b]pyridin-2-yl)pyridin-2-amine

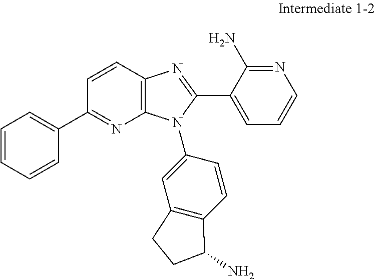 [SMILES: Nc1ncccc1-c1nc2ccc(-c3ccccc3)nc2n1-c1ccc2c(c1)CC[C@H]2N]

### Intermediate 1-3: (S)-3-(3-(1-amino-2,3-dihydro-1H-inden-5-yl)-5-phenyl-3H-imidazo[4,5-b]pyridin-2-yl)pyridin-2-amine

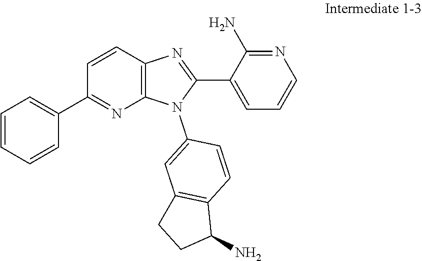 [SMILES: Nc1ncccc1-c1nc2ccc(-c3ccccc3)nc2n1-c1ccc2c(c1)CC[C@@H]2N]

### Synthetic Route:

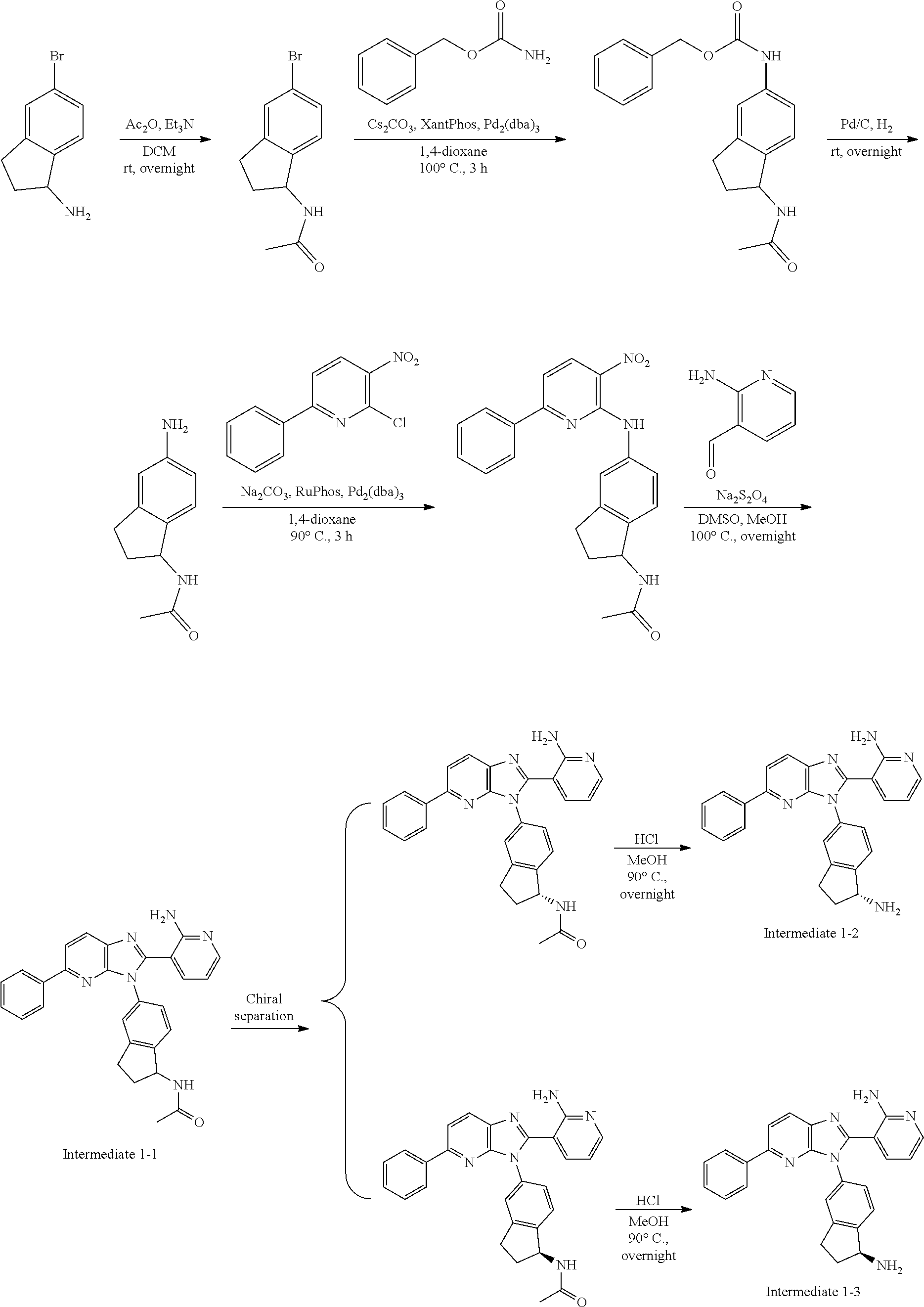

### Step 1: Synthesis of N-(5-bromo-2,3-dihydro-1H-inden-1-yl)acetamide

5-Bromo-2,3-dihydro-1H-inden-1-amine (10 g, 47 mmol, 1 equiv) was dissolved in dichloromethane (500 mL), followed by addition of acetic anhydride (7.2 g, 71 mmol, 1.5 equiv) and triethylamine (14 g, 141 mmol, 3 equiv). The reaction mixture was stirred at room temperature overnight. The reaction was quenched by pouring into water (500 mL) and the mixture was extracted with dichloromethane (2×500 mL). The combined organic layers were dried with anhydrous sodium sulfate, filtered and concentrated. The product was purified by silica gel chromatography using a gradient of 50-80% ethyl acetate in petroleum ether to afford N-(5-bromo-2,3-dihydro-1H-inden-1-yl)acetamide (10 g, 83%) as a white solid. MS (ESI) calculated for C₁₁H₁₂BrNO: 253.01 m/z, found 254.15/256.15 [M+H]⁺.

### Step 2: Synthesis of benzyl N-(1-acetamido-2,3-dihydro-1H-inden-5-yl)carbamate

A microwave tube was charged with Pd₂(dba)₃ (0.54 g, 0.59 mmol, 0.1 equiv), N-(5-bromo-2,3-dihydro-1H-inden-1-yl)acetamide (1.5 g, 5.9 mmol, 1 equiv), O-benzyl carbamate (1.1 g, 7.1 mmol, 1.2 equiv), cesium carbonate (4.8 g, 15 mmol, 2.5 equiv) and 1,4-dioxane (10 mL). The microwave tube was then flushed with nitrogen and evacuated 3 times. The reaction was stirred at 100° C. for 3 h. Six batches were done in parallel. The reaction was quenched with water at room temperature. The resulting mixture was extracted with dichloromethane (3×50 mL). The combined organic layers were washed with brine (50 mL) and dried over anhydrous sodium sulfate, filtered and concentrated in vacuo. The residue was purified by silica gel column chromatography using a gradient of 50-80% ethyl acetate in petroleum ether to afford benzyl N-(1-acetamido-2,3-dihydro-1H-inden-5-yl)carbamate (5.6 g, 78%) as a red/brown oil. MS (ESI) calculated for C₁₉H₂₀N₂O₃: 324.15 m/z, found 325.10 [M+H]⁺.

### Step 3: Synthesis of N-(5-amino-2,3-dihydro-1H-inden-1-yl)acetamide

A round-bottomed flask was charged with benzyl N-(1-acetamido-2,3-dihydro-1H-inden-5-yl)carbamate (5.6 g, 17 mmol, 1 equiv), 10% Pd/C (2.8 g, 26 mmol, 1.5 equiv), methanol (50 mL) and ethyl acetate (200 mL). The reaction mixture was stirred under H₂ at room temperature overnight. The reaction mixture was filtered and concentrated. The product was purified by silica gel chromatography with a 50-80% gradient of ethyl acetate in petroleum ether to afford N-(5-amino-2,3-dihydro-1H-inden-1-yl)acetamide (2.9 g, 88%) as a brown oil. MS (ESI) calculated for C₁₁H₁₄N₂O: 190.11 m/z, found 191.15 [M+H]⁺.

### Step 4: Synthesis of N-{5-[(3-nitro-6-phenylpyridin-2-yl)amino]-2,3-dihydro-1H-inden-1-yl}acetamide

A microwave tube was charged with added Pd₂(dba)₃ (0.48 g, 0.53 mmol, 0.1 equiv), N-(5-amino-2,3-dihydro-1H-inden-1-yl)acetamide (1 g, 5.3 mmol, 1 equiv), 2-chloro-3-nitro-6-phenylpyridine (1.5 g, 6.3 mmol, 1.2 equiv), RuPhos (0.25 g, 0.53 mmol, 0.1 equiv), sodium carbonate (1.1 g, 11 mmol, 2 equiv) and 1,4-dioxane (15 mL). The mixture was stirred at 90° C. for 3 hours under N₂. Three batches were run in parallel. The reaction was quenched with water at room temperature. The resulting mixture was extracted with ethyl acetate (3×50 mL). The combined organic layers were washed with brine (50 mL), dried over anhydrous sodium sulfate, filtered and concentrated in vacuo. The residue was purified by silica gel column chromatography using a gradient of 60-80% ethyl acetate in petroleum ether to afford N-{5-[(3-nitro-6-phenylpyridin-2-yl)amino]-2,3-dihydro-1H-inden-1-yl}acetamide (4.5 g, 83%) as a brown oil. MS (ESI) calculated for C₂₂H₂₀N₄O₃: 388.15 m/z, found 389.15 [M+H]⁺.

### Step 5: Synthesis of N-{5-[2-(2-aminopyridin-3-yl)-5-phenylimidazo[4,5-b]pyridin-3-yl]-2,3-dihydro-1H-inden-1-yl}acetamide (Intermediate 1-1)

A round-bottomed flask was charged with N-{5-[(3-nitro-6-phenylpyridin-2-yl)amino]-2,3-dihydro-1H-inden-1-yl}acetamide (4.5 g, 12 mmol, 1 equiv), 2-aminopyridine-3-carbaldehyde (1.7 g, 14 mmol, 1.2 equiv), sodium dithionite (6.1 g, 35 mmol, 3 equiv), DMSO (180 mL) and methanol (30 mL). The reaction mixture was refluxed at 100° C. overnight. The reaction was quenched by pouring into water (500 mL) and the mixture was extracted with ethyl acetate (3×100 mL). The combined organic fractions were dried with anhydrous sodium sulfate, filtered and concentrated. The residue was purified by reverse-phase flash chromatography on C18 silica gel using a 10-50% gradient of acetonitrile in water (+0.05% ammonium bicarbonate) to afford N-{5-[2-(2-aminopyridin-3-yl)-5-phenylimidazo[4,5-b]pyridin-3-yl]-2,3-dihydro-1H-inden-1-yl}acetamide (Intermediate 1-1) (2.9 g, 89%) as a yellow solid. MS (ESI) calculated for C₂₈H₂₄N₆O: 460.20 m/z, found 461.20 [M+H]⁺.

### Step 6: Chiral separation of N-{5-[2-(2-aminopyridin-3-yl)-5-phenylimidazo[4,5-b]pyridin-3-yl]-2,3-dihydro-1H-inden-1-yl}acetamide

The two enantiomers of N-{5-[2-(2-aminopyridin-3-yl)-5-phenylimidazo[4,5-b]pyridin-3-yl]-2,3-dihydro-1H-inden-1-yl}acetamide (Intermediate 1-1) were separated by chiral Preparative HPLC using a (R, R)-WHELK-O1-Kromasi column (5×25 cm, 5 μm) with 40% ethanol in hexanes (+0.5% 2N ammonia in methanol) to provide N-[(1R)-5-[2-(2-aminopyridin-3-yl)-5-phenylimidazo[4,5-b]pyridin-3-yl]-2,3-dihydro-1H-inden-1-yl]acetamide as the first eluting peak and N-[(1S)-5-[2-(2-aminopyridin-3-yl)-5-phenylimidazo[4,5-b]pyridin-3-yl]-2,3-dihydro-1H-inden-1-yl]acetamide as the second eluting peak.

### Step 7: Synthesis of 3-{3-[(1R)-1-amino-2,3-dihydro-1H-inden-5-yl]-5-phenylimidazo[4,5-b]pyridin-2-yl}pyridin-2-amine (Intermediate 1-2)

To a stirred solution of N-[(1R)-5-[2-(2-aminopyridin-3-yl)-5-phenylimidazo[4,5-b]pyridin-3-yl]-2,3-dihydro-1H-inden-1-yl]acetamide (280 mg, 0.65 mmol, 1 equiv) in methanol (4 mL) was added HCl (0.5 mL, concentrated). The resulting mixture stirred at 90° C. overnight. The mixture was concentrated under reduced pressure to afford 3-{3-[(1R)-1-amino-2,3-dihydro-1H-inden-5-yl]-5-phenylimidazo[4,5-b]pyridin-2-yl}pyridin-2-amine (Intermediate 1-2) (200 mg, 73%) as a red/brown oil, which was used without further purification in subsequent transformations. MS (ESI) calculated for C₂₆H₂₂N₆: 418.19 m/z, found 419.20 [M+H]⁺.

### Step 8: Synthesis of 3-{3-[(1S)-1-amino-2,3-dihydro-1H-inden-5-yl]-5-phenylimidazo[4,5-b]pyridin-2-yl}pyridin-2-amine (Intermediate 1-3)

To a stirred solution of N-[(1S)-5-[2-(2-aminopyridin-3-yl)-5-phenylimidazo[4,5-b]pyridin-3-yl]-2,3-dihydro-1H-inden-1-yl]acetamide (300 mg, 0.65 mmol, 1 equiv) in methanol (4 mL) was added HCl (0.5 mL, concentrated). The resulting mixture stirred at 90° C. overnight. The mixture was concentrated under reduced pressure to afford 3-{3-[(1S)-1-amino-2,3-dihydro-1H-inden-5-yl]-5-phenylimidazo[4,5-b]pyridin-2-yl}pyridin-2-amine (Intermediate 1-3) (200 mg, 73%) as a red/brown oil, which was used without further purification in subsequent transformations. MS (ESI) calculated for C₂₆H₂₂N₆: 418.19 m/z, found 419.20 [M+H]⁺.

### Intermediate 1-4: 3-(3-(1-amino-2,3-dihydro-1H-inden-5-yl)-5-phenyl-3H-imidazo[4,5-b]pyridin-2-yl)pyridin-2-amine

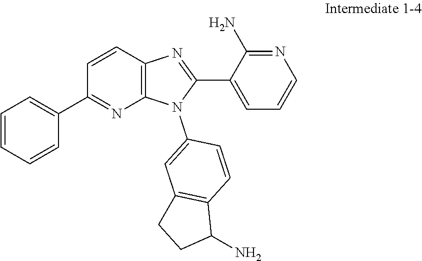 [SMILES: Nc1ncccc1-c1nc2ccc(-c3ccccc3)nc2n1-c1ccc2c(c1)CCC2N]

### Synthetic Route:

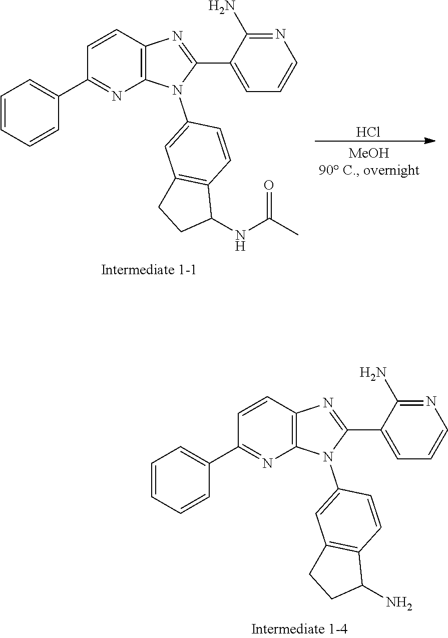 [SMILES: CC(=O)NC1CCc2cc(-n3c(-c4cccnc4N)nc4ccc(-c5ccccc5)nc43)ccc21 ; Cl ; Nc1ncccc1-c1nc2ccc(-c3ccccc3)nc2n1-c1ccc2c(c1)CCC2N]

To a stirred solution of N-(5-(2-(2-aminopyridin-3-yl)-5-phenyl-3H-imidazo[4,5-b]pyridin-3-yl)-2,3-dihydro-1H-inden-1-yl)acetamide (Intermediate 1-1) (300 mg, 0.65 mmol, 1 equiv) in methanol (4 mL) was added HCl (0.5 mL, concentrated). The resulting mixture stirred at 90° C. overnight. The mixture concentrated under reduced pressure to afford 3-(3-(1-amino-2,3-dihydro-1H-inden-5-yl)-5-phenyl-3H-imidazo[4,5-b]pyridin-2-yl)pyridin-2-amine (Intermediate 1-4) (200 mg, 73%) as a red/brown oil, which was used without further purification in subsequent transformations. MS (ESI) calculated for C₂₆H₂₂N₆: 418.19 m/z, found 419.25 [M+H]⁺.

### Intermediate 1-5: 2-(3-((tert-butyldimethylsilyl)oxy)-4-(1,3-dioxolan-2-yl)phenyl)acetaldehyde

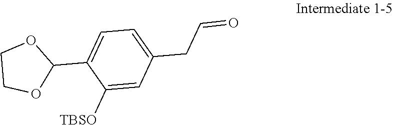 [SMILES: CC(C)(C)[Si](C)(C)Oc1cc(CC=O)ccc1C1OCCO1]

### Synthetic Route:

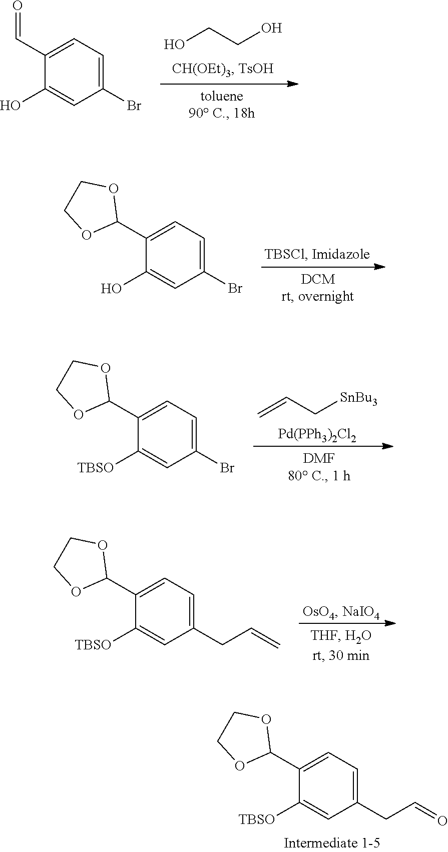

### Step 1: Synthesis of 5-bromo-2-(1,3-dioxolan-2-yl)phenol

To a solution of 4-bromo-2-hydroxybenzaldehyde (10 g, 50 mmol, 1 equiv) in toluene (100 mL) were added p-toluenesulfonic acid (0.86 g, 5 mmol, 0.1 equiv), ethylene glycol (15.4 g, 250 mmol, 5 equiv) and triethyl orthoformate (22.1 g, 150 mmol, 3 equiv). The resulting solution was stirred at room temperature for 10 min then overnight at 90° C. The reaction mixture was cooled to 0° C. and quenched by the addition of saturated aqueous ammonium chloride (80 mL). The resulting mixture was extracted with ethyl acetate (100 mL×3). The combined organic layers were washed with water (100 mL×3), dried over anhydrous sodium sulfate, filtered and concentrate in vacuo. The resulting residue was purified by silica gel column chromatography using a gradient of 0-7% ethyl acetate in petroleum ether to provide 5-bromo-2-(1,3-dioxolan-2-yl)phenol (6 g, 49% yield) as a light-yellow oil. MS (ESI) calculated for C₉H₉BrO₃: 244.97, found 245.95 [M+H]⁺.

### Step 2: Synthesis of 5-bromo-2-(1,3-dioxolan-2-yl)phenoxy(tert-butyl)dimethylsilane

To a solution of 5-bromo-2-(1,3-dioxolan-2-yl)phenol (19 g, 78 mmol, 1 equiv) and imidazole (10.6 g, 155 mmol, 2 equiv) in dichloromethane (200 mL) was added tert-butyldimethylsilyl chloride (16.4 g, 109 mmol, 1.4 equiv). The resulting mixture was stirred overnight at room temperature then cooled to 0° C. and quenched with water (200 mL). The resulting mixture was extracted with ethyl acetate (300 mL×3). The combined organic layers were washed with water (300 mL×3), dried over anhydrous sodium sulfate, filtered and concentrated in vacuo. The resulting residue was purified by silica gel column chromatography using a gradient of (0-7% ethyl acetate in petroleum ether to provide 5-bromo-2-(1,3-dioxolan-2-yl)phenoxy(tert-butyl)dimethylsilane (22 g, 80% yield) as a colorless oil. MS (ESI) calculated for C₁₅H₂₃BrO₃Si: 358.06, found: 359.05 [M+H]⁺.

### Step 3: Synthesis of tert-butyl(2-(1,3-dioxolan-2-yl)-5-(prop-2-en-1-yl)phenoxy)dimethylsilane

To a solution of 5-bromo-2-(1,3-dioxolan-2-yl)phenoxy(tert-butyl)dimethylsilane (1 g, 2.8 mmol, 1 equiv) and tributyl(prop-2-en-1-yl)stannane (1.84 g, 5.6 mmol, 2 equiv) in N,N-dimethylformamide (10 mL) was added Pd(PPh₃)₂Cl₂ (0.20 g, 0.28 mmol, 0.1 equiv). After stirring for 1 h at 80° C. under a nitrogen atmosphere, the mixture was concentrated under reduced pressure. The residue was purified by reverse-phase flash chromatography on C18 silica gel using a 10-40% gradient of acetonitrile in water to give tert-butyl(2-(1,3-dioxolan-2-yl)-5-(prop-2-en-1-yl)phenoxy)dimethylsilane (0.8 g, 90% yield) as colorless oil. MS (ESI) calculated for C₁₈H₂₈O₃Si: 320.18, found 321.20 [M+H]⁺.

### Step 4: Synthesis of 2-{3-[(tert-butyldimethylsilyl)oxy]-4-(1,3-dioxolan-2-yl)phenyl}acetaldehyde (Intermediate 1-5)

To a solution of tert-butyl(2-(1,3-dioxolan-2-yl)-5-(prop-2-en-1-yl)phenoxy)dimethylsilane (1 g, 3.1 mmol, 1 equiv) in acetonitrile (3 mL) and water (3 mL) were added OsO₄ (2.38 g, 9.4 mmol, 3 equiv) and NaIO₄ (2.00 g, 9.4 mmol, 3 equiv). After stirring for 30 min at room temperature, the reaction was quenched by the addition of water. The resulting mixture was extracted with ethyl acetate (20 mL×3). The combined organic layers were washed with water (20 mL×3) and dried over anhydrous sodium sulfate, filtered and concentrated in vacuo. The residue was purified on a silica gel column using a 0-25% gradient of ethyl acetate in petroleum ether to give 2-{3-[(tert-butyldimethylsilyl)oxy]-4-(1,3-dioxolan-2-yl)phenyl}acetaldehyde (Intermediate 1-5) (0.2 g, 20% yield) as a light-yellow oil. MS (ESI) calculated for C₁₇H₂₆O₄Si: 322.16, found 323.15 [M+H]⁺.

### Example 2: 5-(2-((5-(2-(2-aminopyridin-3-yl)-5-phenyl-3H-imidazo[4,5-b]pyridin-3-yl)-2,3-dihydro-1H-inden-1-yl)amino)ethyl)-2-hydroxybenzaldehyde

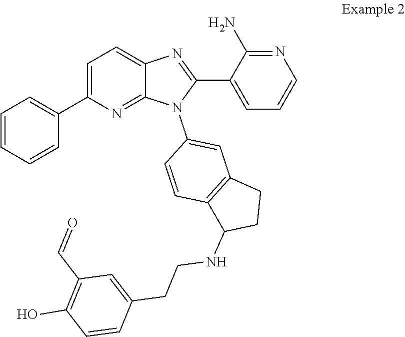 [SMILES: Nc1ncccc1-c1nc2ccc(-c3ccccc3)nc2n1-c1ccc2c(c1)CCC2NCCc1ccc(O)c(C=O)c1]

### Synthetic Route:

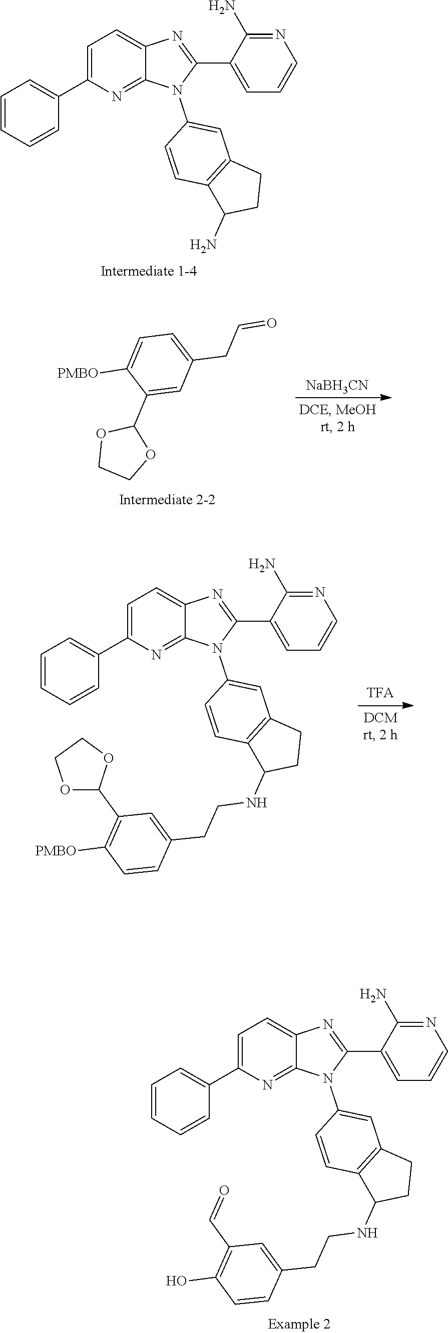

### Step 1: Synthesis of 3-{3-[1-({2-[3-(1,3-dioxolan-2-yl)-4-[(4-methoxyphenyl)methoxy]phenyl]ethyl}amino)-2,3-dihydro-1H-inden-5-yl]-5-phenylimidazo[4,5-b]pyridin-2-yl}pyridin-2-amine

A mixture of 3-[3-(1-amino-2,3-dihydro-1H-inden-5-yl)-5-phenylimidazo[4,5-b]pyridin-2-yl]pyridin-2-amine (Intermediate 1-4) (132 mg, 0.32 mmol, 1 equiv) and 2-[3-(1,3-dioxolan-2-yl)-4-[(4-methoxyphenyl)methoxy]phenyl]acetaldehyde (Intermediate 2-2) (207 mg, 0.63 mmol, 2 equiv) in methanol (6 mL) and 1,2-dichloroethane (6 mL) was stirred for 30 min at room temperature. To the mixture was added NaBH₃CN (59 mg, 0.95 mmol, 3 equiv) and stirring was continued for 1 h. The reaction was quenched with water (10 mL) and the mixture was extracted with dichloromethane (3×20 mL). The combined organic layers were dried over anhydrous sodium sulfate, filtered and concentrated in vacuo. The residue was purified by reverse-phase flash column chromatography on C18 silica gel using a 20 to 60% gradient of acetonitrile in water (+0.05% TFA) to afford 3-{3-[1-({2-[3-(1,3-dioxolan-2-yl)-4-[(4-methoxyphenyl)methoxy]phenyl]ethyl}amino)-2,3-dihydro-1H-inden-5-yl]-5-phenylimidazo[4,5-b]pyridin-2-yl}pyridin-2-amine (88 mg, 38%) as a yellow oil. MS (ESI) calculated for C₄₅H₄₂N₆O₄, 730.33 m/z, found 731 [M+H]⁺.

### Step 2 Synthesis of 5-(2-((5-(2-(2-aminopyridin-3-yl)-5-phenyl-3H-imidazo[4,5-b]pyridin-3-yl)-2,3-dihydro-1H-inden-1-yl)amino)ethyl)-2-hydroxybenzaldehyde (Example 2)

A mixture of 3-{3-[1-({2-[3-(1,3-dioxolan-2-yl)-4-[(4-methoxyphenyl)methoxy] phenyl]ethyl}amino)-2,3-dihydro-1H-inden-5-yl]-5-phenylimidazo[4,5-b]pyridin-2-yl}pyridin-2-amine (50 mg, 0.088 mmol, 1 equiv), TFA (0.5 mL) and dichloromethane (2 mL) was stirred for 2 h at room temperature. After concentration, the residue was purified by preparative HPLC on a XBridge Prep OBD C18 column (30×150 mm, 5 μm) using a 19 to 35% gradient of acetonitrile in water (+0.05% TFA), to afford 5-[2-({5-[2-(2-aminopyridin-3-yl)-5-phenylimidazo[4,5-b]pyridin-3-yl]-2,3-dihydro-1H-inden-1-yl}amino)ethyl]-2-hydroxybenzaldehyde (Example 2) (2.7 mg, 5%) as a yellow solid. MS (ESI) calculated for C₃₅H₃₀N₆O₂: 566.24 m/z, found 567.25 [M+H]⁺. ¹H NMR (400 MHz, DMSO-d₆) δ (ppm): 10.25 (s, 1H), 8.29-8.31 (m, 1H), 8.00-8.03 (m, 4H), 7.73-7.75 (m, 1H), 7.55-7.59 (m, 2H), 7.39-7.49 (m, 6H), 6.98-7.00 (m, 1H), 6.55-6.58 (m, 1H), 4.87-4.90 (m, 1H), 3.20-3.25 (m, 3H), 2.89-3.14 (m, 3H), 2.54-2.63 (m, 1H), 2.22-2.34 (m, 1H).

### Intermediate 2-2: 2-[3-(1,3-dioxolan-2-yl)-4-[(4-methoxyphenyl)methoxy]phenyl]acetaldehyde

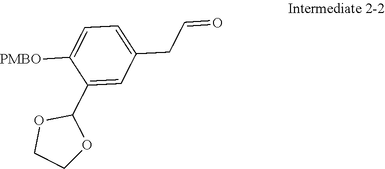

### Synthetic Route:

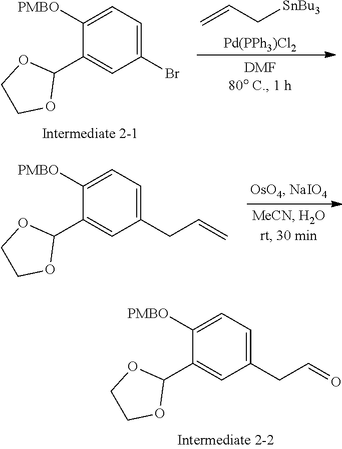

### Step 1: Synthesis of 2-(5-allyl-2-((4-methoxybenzyl)oxy)phenyl)-1,3-dioxolane

To a solution of 2-(5-bromo-2-((4-methoxybenzyl)oxy)phenyl)-1,3-dioxolane (Intermediate 2-1) (3 g, 8.2 mmol, 1 equiv) and tributyl(prop-2-en-1-yl)stannane (5.44 g, 16.4 mmol, 2 equiv) in N,N-dimethylformamide (24 mL) was added Pd(PPh₃)₂Cl₂ (0.58 g, 0.82 mmol, 0.1 equiv). After stirring for 1 h at 80° C. under a nitrogen atmosphere, the mixture was concentrated under reduced pressure. The residue was purified by reverse-phase flash chromatography on C18 silica gel using a 10 to 40% gradient of acetonitrile to provide 2-(5-allyl-2-((4-methoxybenzyl)oxy)phenyl)-1,3-dioxolane (2.4 g, 90% yield) as colorless oil. MS (ESI) calculated for C₂₀H₂₂O₄: 326.15, found 327.20 [M+H]⁺.

### Step 2: Synthesis of 2-(3-(1,3-dioxolan-2-yl)-4-((4-methoxybenzyl)oxy)phenyl)acetaldehyde (Intermediate 2-2)

To a solution of 2-(5-allyl-2-((4-methoxybenzyl)oxy)phenyl)-1,3-dioxolane (0.5 g, 1.53 mmol, 1 equiv) in acetonitrile (3 mL) and water (3 mL) were added OsO4 (1.17 g, 4.60 mmol, 3 equiv) and NaIO4 (0.98 g, 4.60 mmol, 3 equiv). After stirring for 30 min at room temperature, the reaction was quenched by the addition of water. The resulting mixture was extracted with ethyl acetate (20 mL×3). The combined organic layers were washed with water (20 mL×3), dried over anhydrous sodium sulfate, filtered and concentrated in vacuo. The residue was purified by silica gel column chromatography using a gradient of 0-25% ethyl acetate in petroleum ether to provide 2-(3-(1,3-dioxolan-2-yl)-4-((4-methoxybenzyl)oxy)phenyl) acetaldehyde (Intermediate 2-2) (0.15 g, 30% yield) as a light-yellow oil. MS (ESI) calculated For C19H20O5: 328.13, found 329.15 [M+H]+.

### Intermediate 2-1: 2-{5-bromo-2-[(4-methoxyphenyl)methoxy]phenyl}-1,3-dioxolane

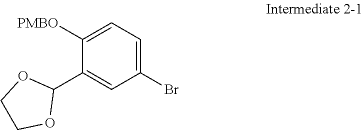

### Synthetic Route:

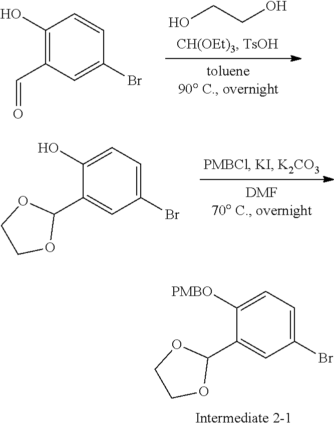

### Step 1: Synthesis of 4-bromo-2-(1,3-dioxolan-2-yl)phenol

A solution of 5-bromo-2-hydroxybenzaldehyde (20 g, 100 mmol, 1 equiv), ethylene glycol (31 g, 497 mmol, 5 equiv), triethyl orthoformate (44 g, 298 mmol, 3 equiv) and p-toluenesulfonic acid (1.71 g, 9.93 mmol, 0.10 equiv) in toluene (400 mL) was stirred at 90° C. overnight. The reaction mixture was cooled to room temperature and water (500 mL) was added. The resulting mixture was extracted with ethyl acetate (500 mL×3). The combined organic layers were washed with brine (500 mL×2), dried with anhydrous sodium sulfate, filtered and concentrated in vacuo. The residue was purified by silica gel column chromatography using a gradient of ethyl acetate in petroleum ether to provide 4-bromo-2-(1,3-dioxolan-2-yl)phenol as an off-white solid (20 g, 62% yield). MS (ESI) calculated for C₉H₉BrO₃: 243.97 m/z, found 244.90, 246.90 [M+H, M+H+2]⁺.

### Step 2: Synthesis of 2-{5-bromo-2-[(4-methoxyphenyl)methoxy]phenyl}-1,3-dioxolane (Intermediate 2-1)

A suspension of 4-bromo-2-(1,3-dioxolan-2-yl)phenol (10 g, 40.8 mmol, 1 equiv), potassium carbonate (16.92 g, 122 mmol, 3 equiv), potassium iodide (0.68 g, 4.08 mmol, 0.1 equiv) and para-methoxybenzyl chloride (7.67 g, 49 mmol, 1.2 equiv) in N,N-dimethylformamide (60 mL) was stirred at 70° C. overnight. The resulting mixture was cooled to room temperature and water (200 mL) was added. The mixture was then extracted with ethyl acetate (100 mL×3). The combined organic layers were washed with brine (200 mL×2), dried with anhydrous sodium sulfate, filtered and concentrated in vacuo. The residue was purified by silica gel column chromatography using a gradient of ethyl acetate in petroleum ether to provide 2-{5-bromo-2-[(4-methoxyphenyl)methoxy]phenyl}-1,3-dioxolane (Intermediate 2-1) as an off-white solid (7 g, 43% yield). MS (ESI) calculated for C17H17BrO4: 364.03 m/z, found 366.95, 388.95 [M+H, M+Na+2]⁺. ¹H NMR (300 MHz, DMSO-d6) δ 7.47-7.55 (m, 2H), 7.35-7.43 (m, 2H), 7.07-7.15 (m, 1H), 6.91-7.00 (m, 2H), 5.99 (s, 1H), 5.08 (s, 2H), 3.99-4.09 (m, 2H), 3.87-3.97 (m, 2H), 3.76 (s, 3H).

### Example 3: 4-(2-{[(1R)-5-[2-(2-aminopyridin-3-yl)-5-phenylimidazo[4,5-b]pyridin-3-yl]-2,3-dihydro-1H-inden-1-yl]amino}ethyl)-2-hydroxybenzaldehyde

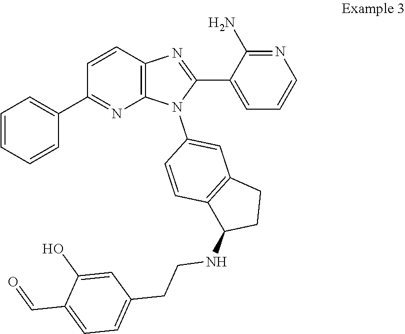 [SMILES: Nc1ncccc1-c1nc2ccc(-c3ccccc3)nc2n1-c1ccc2c(c1)CC[C@H]2NCCc1ccc(C=O)c(O)c1]

Example 3 was prepared in a manner analogous to Example 1 using Intermediate 1-2 in place of Intermediate 1-4. MS (ESI) calculated for C₃₅H₃₀N₆O₂: 566.24 m/z, found 567.20[M+H]⁺. ¹H NMR (300 MHz, DMSO-d₆) δ (ppm): 10.15 (s, 1H), 8.23-8.26 (m, 1H), 7.94-8.01 (m, 4H), 7.58-7.61 (m, 1H), 7.44-7.48 (m, 4H), 7.39-7.41 (m, 1H), 7.19-7.32 (m, 2H), 6.87-6.90 (m, 2H), 6.36-6.40 (m, 1H), 4.20-4.30 (m, 1H), 2.77-2.91 (m, 5H), 2.34-2.38 (m, 2H), 1.80-1.83 (m, 1H).

### Example 4: 4-(2-{[(1S)-5-[2-(2-aminopyridin-3-yl)-5-phenylimidazo[4,5-b]pyridin-3-yl]-2,3-dihydro-1H-inden-1-yl]amino}ethyl)-2-hydroxybenzaldehyde

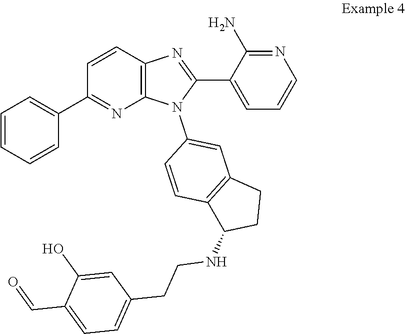 [SMILES: Nc1ncccc1-c1nc2ccc(-c3ccccc3)nc2n1-c1ccc2c(c1)CC[C@@H]2NCCc1ccc(C=O)c(O)c1]

Example 4 was prepared in a manner analogous to Example 1 using Intermediate 1-3 in place of Intermediate 1-4. MS (ESI) calculated for C₃₅H₃₀N₆O₂: 566.24 m/z, found 567.20[M+H]⁺. ¹H NMR (300 MHz, DMSO-d₆) δ (ppm): 10.15 (s, 1H), 8.24-8.27 (m, 1H), 7.95-8.00 (m, 4H), 7.59-7.62 (m, 1H), 7.19-7.49 (m, 7H), 6.87-6.90 (m, 2H), 6.36-6.40 (m, 1H), 4.20-4.30 (m, 1H), 2.77-2.86 (m, 5H), 2.35-2.50 (m, 2H), 1.70-1.90 (m, 1H).

### Example 5: (S)—N-(5-(2-(2-aminopyridin-3-yl)-5-phenyl-3H-imidazo[4,5-b]pyridin-3-yl)-2,3-dihydro-1H-inden-1-yl)-3-formyl-4-hydroxybenzamide

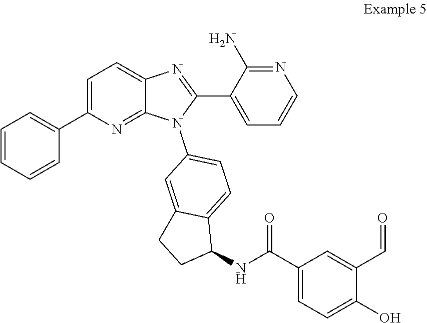 [SMILES: Nc1ncccc1-c1nc2ccc(-c3ccccc3)nc2n1-c1ccc2c(c1)CC[C@@H]2NC(=O)c1ccc(O)c(C=O)c1]

### Synthetic Route:

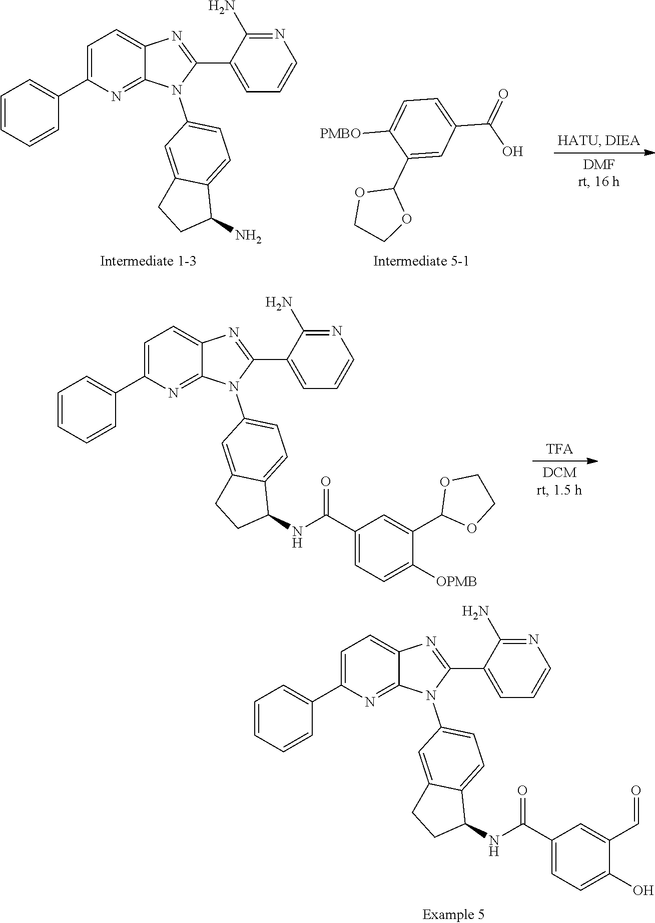

### Step 1: Synthesis of (S)—N-(5-(2-(2-aminopyridin-3-yl)-5-phenyl-3H-imidazo[4,5-b]pyridin-3-yl)-2,3-dihydro-1H-inden-1-yl)-3-(1,3-dioxolan-2-yl)-4-((4-methoxybenzyl)oxy)benzamide

To a solution of 3-{3-[(1S)-1-amino-2,3-dihydro-1H-inden-5-yl]-5-phenylimidazo[4,5-b]pyridin-2-yl}pyridin-2-amine (Intermediate 1-3) (60 mg, 0.14 mmol, 1 equiv), 3-(1,3-dioxolan-2-yl)-4-[(4-methoxyphenyl)methoxy]benzoic acid (Intermediate 5-1) (57 mg, 0.17 mmol, 1.2 equiv) and N,N-diisopropylethylamine (111 mg, 0.86 mmol, 6 equiv) in N,N-dimethylformamide (3.4 mL) was added HATU (14 mg, 0.036 mmol, 1.5 equiv). The resulting mixture was stirred at 25° C. for 16 h. Water was added, and the mixture was extracted with ethyl acetate (10 mL×3). The combined organic layers were washed with brine (10 mL×3), dried with anhydrous sodium sulfate, filtered and concentrated in vacuo. The residue was purified by silica gel column chromatography using a 0-10% gradient of methanol in dichloromethane to afford N-[(1S)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-[(4-methoxyphenyl)methoxy]benzamide (70 mg, 61%) as a yellow solid. MS (ESI) calculated for C₄₄H₃₈N₆O₅: 730.29 m/z, found 731.15 [M+H]⁺.

### Step 2: Synthesis of (S)—N-(5-(2-(2-aminopyridin-3-yl)-5-phenyl-3H-imidazo[4,5-b]pyridin-3-yl)-2,3-dihydro-1H-inden-1-yl)-3-formyl-4-hydroxybenzamide (Example 5)

A solution of N-[(1S)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-[(4-methoxyphenyl)methoxy]benzamide (70 mg, 0.096 mmol, 1 equiv) in dichloromethane (5 mL) and TFA (1 mL) was stirred at 25° C. for 1.5 h. After concentration, the residue was purified by Preparative HPLC on a Xselect CSH C18 OBD column (30×150 mm, 5 μm) using a 26-48% gradient of acetonitrile in water (+0.05% TFA) to provide N-[(1S)-5-[2-(2-aminopyridin-3-yl)-5-phenylimidazo[4,5-b]pyridin-3-yl]-2,3-dihydro-1H-inden-1-yl]-3-formyl-4-hydroxybenzamide (Example 5) (19.6 mg, 36%) as a yellow solid. MS (ESI) calculated for C₃₄H₂₆N₆O₃: 566.21 m/z, found 567.15 [M+H]⁺. ¹H NMR (400 MHz, DMSO-d₆) δ (ppm): δ 10.31 (s, 1H), 8.28-8.36 (m, 2H), 7.99-8.14 (m, 5H), 7.65-7.74 (m, 1H), 7.33-7.54 (m, 6H), 7.05-7.12 (m, 1H), 6.70-6.80 (m, 1H), 5.58-5.67 (m, 1H), 2.99-3.09 (m, 1H), 2.85-2.96 (m, 1H), 2.50-2.49 (m, 1H), 2.05-2.21 (m, 1H).

### Intermediate 5-1: 3-(1,3-dioxolan-2-yl)-4-((4-methoxybenzyl)oxy)benzoic acid

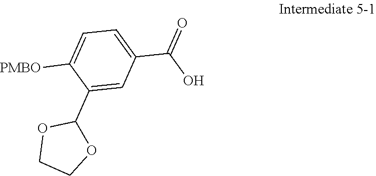

### Synthetic Route:

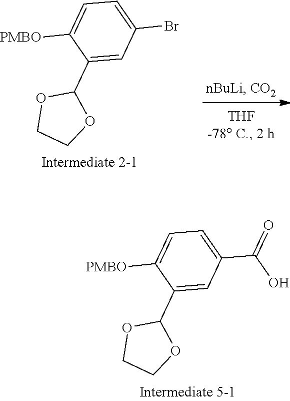

n-Butyllithium (2.5M in hexanes, 3.3 mL, 8.2 mmol, 3 equiv) was added dropwise to a solution of 2-(5-bromo-2-((4-methoxybenzyl)oxy)phenyl)-1,3-dioxolane (Intermediate 2-1) (1 g, 2.7 mmol, 1 equiv) in tetrahydrofuran (20 mL) at −78° C. The resulting solution was stirred at −78° C. for 2 h. Then carbon dioxide was passed through the solution using a needle for 10 min. The reaction was quenched with saturated aqueous ammonium chloride (10 mL). The mixture was extracted with ethyl acetate (20 mL×3). The combined organic layers were washed with brine (30 mL), dried with anhydrous sodium sulfate, filtered and concentrated in vacuo. The residue was purified by reverse-phase column chromatography using a 5-45% gradient of acetonitrile in water (+0.05% ammonium bicarbonate) to provide 3-(1,3-dioxolan-2-yl)-4-((4-methoxybenzyl)oxy)benzoic acid (Intermediate 5-1) (400 mg, 44%) as a light-yellow oil. MS (ESI) calculated for C₁₈H₁₈O₆: 330.11 m/z, found 329.05 [M−H]⁻.

### Example 6: N-(5-(2-(2-aminopyridin-3-yl)-5-phenyl-3H-imidazo[4,5-b]pyridin-3-yl)-2,3-dihydro-1H-inden-1-yl)-2-(4-formyl-3-hydroxyphenyl)acetamide

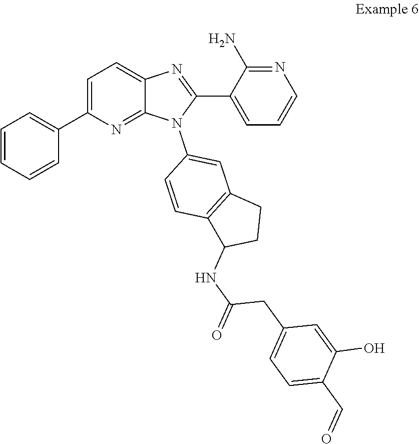 [SMILES: Nc1ncccc1-c1nc2ccc(-c3ccccc3)nc2n1-c1ccc2c(c1)CCC2NC(=O)Cc1ccc(C=O)c(O)c1]

Example 6 was prepared in a manner analogous to Example 5 using Intermediate 1-4 in place of Intermediate 1-3 and Intermediate 6-2 in place of Intermediate 5-1. MS (ESI) calculated for C₃₅H₂₈N₆O₃: 580.22 m/z, found 581.25 [M+H]⁺. ¹H NMR (300 MHz, DMSO-d₆) δ (ppm): 10.18 (s, 1H), 8.31-8.34 (m, 1H), 8.01-8.07 (m, 4H), 7.62-7.74 (m, 2H), 7.28-7.52 (m, 6H), 6.92-6.97 (m, 2H), 6.76-6.80 (s, 1H), 5.32-5.37 (s, 1H), 3.54 (s, 1H), 2.86-2.98 (s, 2H), 2.47-2.52 (s, 1H), 1.89-1.94 (s, 1H).

### Intermediate 6-2: 2-(4-formyl-3-((4-methoxybenzyl)oxy)phenyl)acetic acid

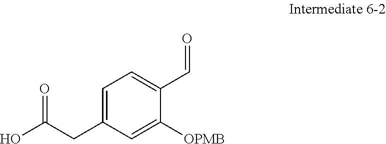

### Synthetic Route:

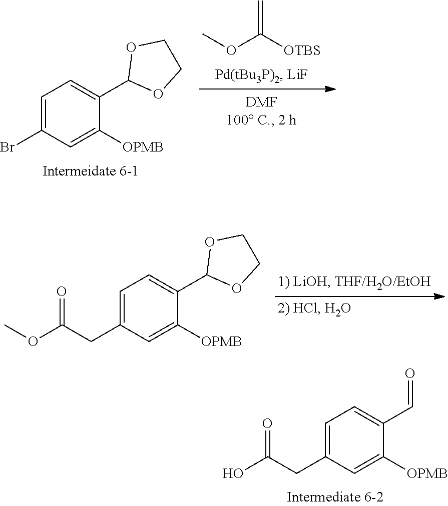

### Step 1: Synthesis of methyl 2-(4-(1,3-dioxolan-2-yl)-3-((4-methoxybenzyl)oxy)phenyl)acetate

To a solution of 2-(4-bromo-2-((4-methoxybenzyl)oxy)phenyl)-1,3-dioxolane (Intermediate 6-1) (4.15 g, 11.3 mmol, 1 equiv) and tert-butyl((1-methoxyvinyl)oxy)dimethylsilane (8.65 g, 45.4 mmol, 4 equiv) in N,N-dimethylformamide (20 mL) were added Pd(tBu₃P)₂ (0.6 g, 1.1 mmol, 0.1 equiv) and lithium fluoride (0.6 g, 22.7 mmol, 2 equiv). The resulting mixture was stirred for 2 h at 100° C. under nitrogen atmosphere. The mixture was cooled to 0° C. and quenched with water (100 mL). The resulting mixture was extracted with ethyl acetate (200 mL×3). The combined organic layers were washed with water (100 mL×3), dried over anhydrous sodium sulfate, filtered and concentrated in vacuo. The resulting residue was purified by silica gel column chromatography using a 0-60% gradient of ethyl acetate in petroleum ether to provide methyl 2-(4-(1,3-dioxolan-2-yl)-3-((4-methoxybenzyl)oxy)phenyl)acetate (3.0 g, 74% yield) as a light-yellow oil. MS (ESI) calculated for C₂₀H₂₂O₆: 358.14, found 359.10 [M+H]⁺.

### Step 2: Synthesis of 2-(4-formyl-3-((4-methoxybenzyl)oxy)phenyl)acetic acid (Intermediate 6-2)

To a cooled (0° C.) solution of methyl 2-(4-(1,3-dioxolan-2-yl)-3-((4-methoxybenzyl)oxy)phenyl)acetate (1.5 g, 4.2 mmol, 1 equiv) in tetrahydrofuran (25 mL) and ethanol (25 mL) was added a solution of lithium hydroxide (0.31 g, 13 mmol, 3 equiv) in water (6.0 mL). The resulting solution was stirred at room temperature for 2 h. The resulting mixture was concentrated under reduced pressure. The residue was suspended in water (5 mL) and acidified to pH 4 with HCl (2N, aqueous). The resulting precipitate was filtered and dried to provide 2-(4-formyl-3-((4-methoxybenzyl)oxy)phenyl)acetic acid (Intermediate 6-2) (1.0 g, 80%) as a light-yellow solid. MS (ESI) calculated for C17H16O5: 300.10, found 301.15 [M+H]+.

### Intermediate 6-1: 2-(4-bromo-2-((4-methoxybenzyl)oxy)phenyl)-1,3-dioxolane

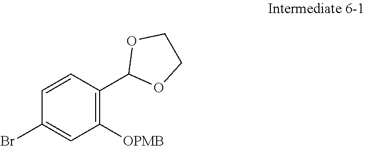

Intermediate 6-1 was prepared in a manner analogous to Intermediate 2-1 using 4-bromo-2-hydroxybenzaldehyde in place of 5-bromo-2-hydroxybenzaldehyde. MS (ESI) calculated for C₁₇H₁₇BrO₄: 364.03 m/z, found 366.95, 388.95 [M+H, M+Na+2]⁺.

### Example 7: N-[(1R)-5-[2-(2-aminopyridin-3-yl)-5-phenylimidazo[4,5-b]pyridin-3-yl]-2,3-dihydro-1H-inden-1-yl]-3-formyl-4-hydroxybenzamide

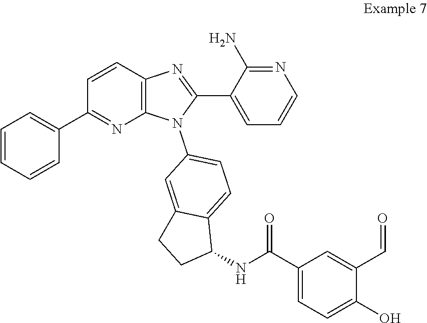 [SMILES: Nc1ncccc1-c1nc2ccc(-c3ccccc3)nc2n1-c1ccc2c(c1)CC[C@H]2NC(=O)c1ccc(O)c(C=O)c1]

### Synthetic Route:

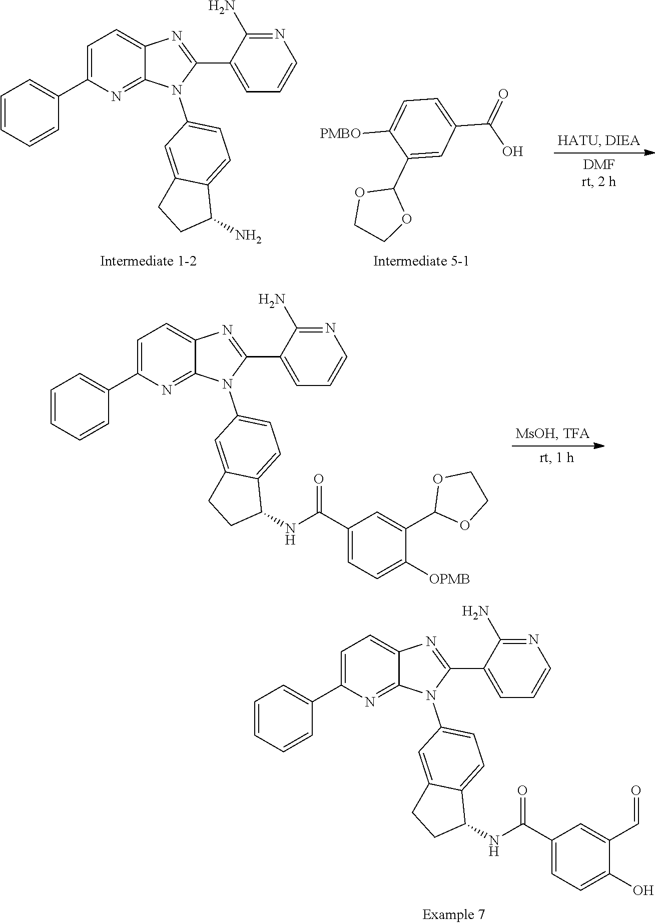

### Step 1: Synthesis of N-[(1R)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-[(4-methoxyphenyl)methoxy]benzamide

To a stirred solution of 3-(1,3-dioxolan-2-yl)-4-[(4-methoxyphenyl)methoxy]benzoic acid (Intermediate 5-1) (80 mg, 0.24 mmol, 1 equiv) in N,N-dimethylformamide (2 mL) were added 3-{3-[(1R)-1-amino-2,3-dihydro-1H-inden-5-yl]-5-phenylimidazo[4,5-b]pyridin-2-yl}pyridin-2-amine (Intermediate 1-2) (101 mg, 0.24 mmol, 1 equiv), HATU (111 mg, 0.29 mmol, 1.2 equiv) and N,N-diisopropylethylamine (63 mg, 0.48 mmol, 2 equiv). The mixture was stirred at room temperature for 2 h. The mixture was purified by reverse-phase flash chromatography on C18 silica gel using a 5 to 70% gradient of acetonitrile in water (+0.05% ammonium bicarbonate) to afford N-[(1R)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-[(4-methoxyphenyl)methoxy]benzamide (150 mg, 85%) as a yellow solid. MS (ESI) calculated for C₄₄H₃₈N₆O₅: 730.29 m/z, found 731.35 [M+H]⁺.

### Step 2: Synthesis of N-[(1R)-5-[2-(2-aminopyridin-3-yl)-5-phenylimidazo[4,5-b]pyridin-3-yl]-2,3-dihydro-1H-inden-1-yl]-3-formyl-4-hydroxybenzamide (Example 7)

A solution of N-[(1R)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-[(4-methoxyphenyl)methoxy]benzamide (100 mg, 0.14 mmol, 1 equiv) in TFA (3 mL) and methanesulfonic acid (1 mL) was stirred for 1 h. The reaction was adjusted to pH 8 with aqueous sodium bicarbonate. The resulting mixture was extracted with ethyl acetate (3×10 mL). The combined organic layers were washed with brine (25 mL), dried over anhydrous sodium sulfate, filtered and concentrated in vacuo. The crude product was purified by preparative HPLC on a Xselect CSH C18 OBD column (30×150 mm 5 μm) using a 29-47% gradient of acetonitrile in water (+0.1% TFA) to afford N-[(1R)-5-[2-(2-aminopyridin-3-yl)-5-phenylimidazo[4,5-b]pyridin-3-yl]-2,3-dihydro-1H-inden-1-yl]-3-formyl-4-hydroxybenzamide (Example 7) (4.9 mg, 6%) as a yellow solid. MS (ESI) calculated for C₃₄H₂₆N₆O₃: 566.21 m/z, found 567.15[M+H]⁺. ¹H NMR (300 MHz, DMSO-d₆) δ (ppm): 10.30 (s, 1H), 8.96-8.99 (m, 1H), 8.11-8.31 (m, 4H), 7.96-8.10 (m, 4H), 7.35-7.51 (m, 6H), 7.26-7.35 (m, 1H), 7.05-7.09 (m, 1H), 6.44-6.49 (m, 1H), 5.62-5.66 (m, 1H), 2.89-3.02 (m, 2H), 2.30-2.40 (m, 1H), 2.06-2.14 (m, 1H).

### Example 8: N-[(1S)-5-[2-(2-aminopyridin-3-yl)-5-phenylimidazo[4,5-b]pyridin-3-yl]-2,3-dihydro-1H-inden-1-yl]-3-fluoro-5-formyl-4-hydroxybenzamide

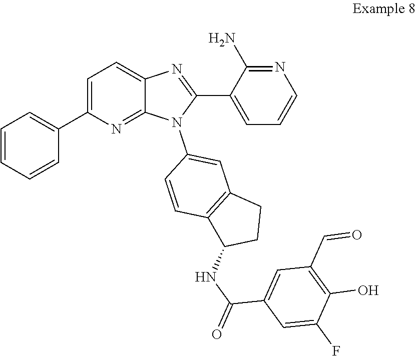 [SMILES: Nc1ncccc1-c1nc2ccc(-c3ccccc3)nc2n1-c1ccc2c(c1)CC[C@@H]2NC(=O)c1cc(F)c(O)c(C=O)c1]

### Synthetic Route:

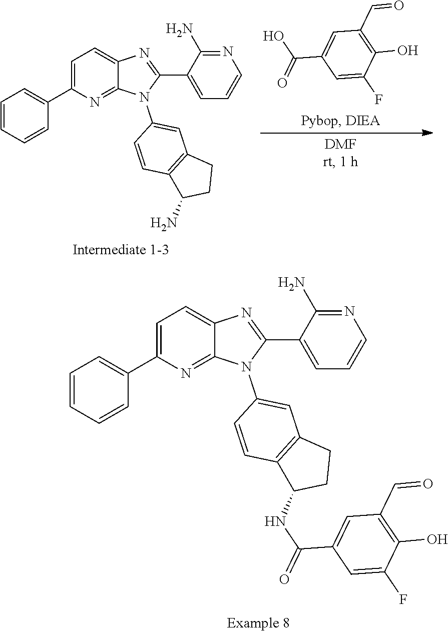

### Step 1: Synthesis of N-[(1S)-5-[2-(2-aminopyridin-3-yl)-5-phenylimidazo[4,5-b]pyridin-3-yl]-2,3-dihydro-1H-inden-1-yl]-3-fluoro-5-formyl-4-hydroxybenzamide (Example 8)

To a solution of 3-{3-[(1S)-1-amino-2,3-dihydro-1H-inden-5-yl]-5-phenylimidazo[4,5-b]pyridin-2-yl}pyridin-2-amine (Intermediate 1-3) (273 mg, 0.65 mmol, 1.5 equiv) in N,N-dimethylformamide (3 mL) were added N,N-diisopropylethylamine (281 mg, 2.2 mmol, 5 equiv), Pybop (339 mg, 0.65 mmol, 1.5 equiv) and 3-fluoro-5-formyl-4-hydroxybenzoic acid (80 mg, 0.43 mmol, 1 equiv). The resulting mixture was stirred at room temperature for 1 h. The mixture was purified by Preparative HPLC on a Xselect CSH C18 OBD column (30×150 mm 5 μm) using a 28-52% gradient of acetonitrile in water (+0.1% formic acid) to afford N-[(1S)-5-[2-(2-aminopyridin-3-yl)-5-phenylimidazo[4,5-b]pyridin-3-yl]-2,3-dihydro-1H-inden-1-yl]-3-fluoro-5-formyl-4-hydroxybenzamide (Example 8) (6.2 mg, 2%) as a yellow solid. MS (ESI) calculated for C₃₄H₂₅FN₆O₃: 584.20 m/z, found 585.15 [M+H]⁺. ¹H-NMR (400 MHz, DMSO-d₆) δ (ppm): 10.28 (s, 1H), 8.95-8.97 (m, 1H), 8.25-8.27 (m, 1H), 8.13-8.14 (m, 1H), 7.97-8.03 (m, 5H), 7.25-7.49 (m, 7H), 6.44-6.47 (m, 1H), 5.62-5.64 (m, 1H), 2.88-3.02 (m, 2H), 2.51 (s, 1H), 2.06-2.12 (m, 1H).

### Example 9: N-[(1S)-5-[2-(2-aminopyridin-3-yl)-5-phenylimidazo[4,5-b]pyridin-3-yl]-2,3-dihydro-1H-inden-1-yl]-2-(2-chloro-4-formyl-3-hydroxyphenyl)acetamide

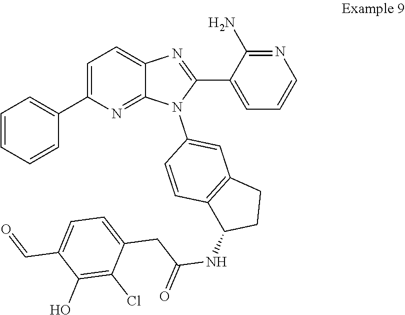 [SMILES: Nc1ncccc1-c1nc2ccc(-c3ccccc3)nc2n1-c1ccc2c(c1)CC[C@@H]2NC(=O)Cc1ccc(C=O)c(O)c1Cl]

Example 9 was prepared in a manner analogous to Example 7 using Intermediate 1-3 in place of Intermediate 1-2 and Intermediate 9-2 in place of Intermediate 5-1. MS (ESI) calculated for C₃₅H₂₇ClN₆O₃: 614.18 m/z, found 615.20 [M+H]⁺. ¹H NMR (300 MHz, DMSO-d₆) δ (ppm): 9.95-10.25 (s, 1H), 8.24-8.27 (m, 1H), 7.90-8.01 (m, 4H), 7.24-7.48 (m, 8H), 6.42-6.80 (m, 2H), 5.35-5.45 (m, 1H), 3.55-3.66 (m, 2H), 2.93-2.96 (m, 1H), 2.85-2.88 (m, 1H), 2.30-2.40 (m, 1H), 1.91-1.95 (m, 1H).

### Intermediate 9-2: [2-chloro-4-(1,3-dioxolan-2-yl)-3-[(4-methoxy phenyl)methoxy]phenyl]acetic

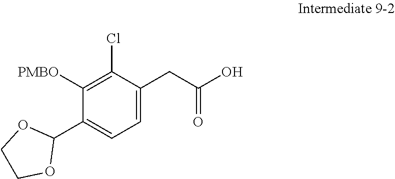

### Synthetic Route:

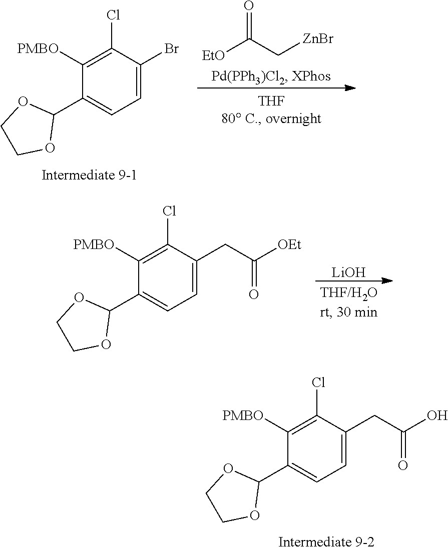

### Step 1: Synthesis of ethyl 2-(2-chloro-4-(1,3-dioxolan-2-yl)-3-((4-methoxybenzyl)oxy)phenyl)acetate

A mixture of 2-(4-bromo-3-chloro-2-((4-methoxybenzyl)oxy)phenyl)-1,3-dioxolane (Intermediate 9-1) (1 g, 2.5 mmol, 1 equiv), Pd(dppf)Cl₂ (0.18 g, 0.25 mmol, 0.1 equiv) and XPhos (CAS 564483-18-7) (0.24 g, 0.500 mmol, 0.2 equiv) in a solution of (2-ethoxy-2-oxoethyl)zinc(II) bromide (30 mL, 129 mmol) in tetrahydrofuran was stirred overnight at 80° C. under N₂ atmosphere. After concentration, the residue was purified by reverse-phase flash column chromatography using a 5 to 80% gradient of acetonitrile in water (+0.05% ammonium bicarbonate) to afford ethyl 2-(2-chloro-4-(1,3-dioxolan-2-yl)-3-((4-methoxybenzyl)oxy)phenyl)acetate (550 mg, 48%) as a brown oil. MS (ESI) calculated for C₂₁H₂₃ClO₆: 406.12 m/z, found 407.10 [M+H]⁺.

### Step 2: Synthesis of [2-chloro-4-(1,3-dioxolan-2-yl)-3-[(4-methoxy phenyl)methoxy]phenyl]acetic acid (Intermediate 9-2)

To a solution of ethyl 2-[2-chloro-4-(1,3-dioxolan-2-yl)-3-[(4-methoxyphenyl)methoxy]phenyl]acetate (300 mg, 0.76 mmol, 1 equiv) in tetrahydrofuran (10 mL) and water (10 mL) was added a solution of lithium hydroxide (55 mg, 2.3 mmol, 3 equiv) in water (1 mL). The mixture was stirred at room temperature for 0.5 h. The reaction mixture was quenched by addition of water (50 mL) and extracted with ethyl acetate. The aqueous layer was adjusted to pH 7 with HCl (2N, aqueous) and extracted with ethyl acetate (50 mL). The combined organic layers were dried over anhydrous sodium sulfate, filtered and concentrated in vacuo to afford crude [2-chloro-4-(1,3-dioxolan-2-yl)-3-[(4-methoxy phenyl)methoxy]phenyl]acetic acid (Intermediate 9-2) (300 mg, 67%) as a yellow solid, which was used without further purification in the next step. MS (ESI) calculated for C₁₉H₁₉ClO₆: 378.09 m/z, found 333.09 [M+H−44]⁺.

### Intermediate 9-1: 2-(4-bromo-3-chloro-2-((4-methoxybenzyl)oxy)phenyl)-1,3-dioxolane

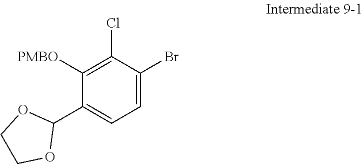

### Synthetic Route:

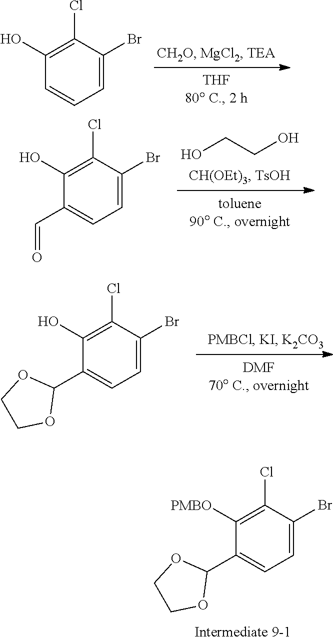

### Step 1: Synthesis of 4-bromo-3-chloro-2-hydroxybenzaldehyde

To a solution of 3-bromo-2-chlorophenol (5 g, 24 mmol, 1 equiv) in tetrahydrofuran (50 mL) were added paraformaldehyde (11 g, 121 mmol, 5 equiv), magnesium chloride (3.4 g, 36 mmol, 1.5 equiv) and triethylamine (6 g, 60 mmol, 2.5 equiv). The resulting suspension was stirred for 2 h at 80° C. The mixture was then cooled to room temperature and filtered, rinsing with methanol (50 mL×3). The filtrate was concentrated under reduced pressure and the residue was recrystallized from methanol/ethyl acetate (10 mL/200 mL) to provide 4-bromo-3-chloro-2-hydroxybenzaldehyde (3 g, 59% yield) as a light yellow solid. MS (ESI) calculated for C₇H₄BrClO₂: 233.91 m/z, found 232.95 [M−H]⁻.

### Step 2: Synthesis of 3-bromo-2-chloro-6-(1,3-dioxolan-2-yl)phenol

To a solution of 4-bromo-3-chloro-2-hydroxybenzaldehyde (3 g, 13 mmol, 1 equiv) in toluene (30 mL) was added ethylene glycol (5.56 g, 89 mmol, 7 equiv), triethyl orthoformate (5.67 g, 38 mmol, 3 equiv) and p-toluenesulfonic acid (0.12 g, 0.64 mmol, 0.05 equiv) at room temperature. The resulting solution was stirred at room temperature for 10 min and at 90° C. overnight. The solution was concentrated, and the residue purified by flash column chromatography on silica gel using a 0-30% gradient of ethyl acetate in petroleum ether to afford 3-bromo-2-chloro-6-(1,3-dioxolan-2-yl)phenol (2 g, 55%) as a yellow oil. MS (ESI) calculated for C₉H₈BrClO₃: 277.93 m/z, found 279.10 [M+H]⁺.

### Step 3: Synthesis of 2-(4-bromo-3-chloro-2-((4-methoxybenzyl)oxy)phenyl)-1,3-dioxolane (Intermediate 9-1)

A mixture of 3-bromo-2-chloro-6-(1,3-dioxolan-2-yl)phenol (2 g, 7.2 mmol, 1 equiv), 4-methoxybenzyl chloride (1.34 g, 8.59 mmol, 1.2 equiv), potassium iodide (0.89 g, 0.72 mmol, 0.1 equiv) and potassium carbonate (2.97 g, 21.5 mmol, 3 equiv) in N,N-dimethylformamide (20 mL) was stirred at 70° C. overnight under N₂. The mixture was cooled to room temperature, diluted with water (40 mL) and extracted with ethyl acetate (50 mL×3). The combined organic layers were washed with brine (100 mL×3), dried with anhydrous sodium sulfate, filtered and concentrated in vacuo. The residue was purified by silica gel column chromatography using a 0-5% gradient of ethyl acetate in petroleum ether to afford 2-(4-bromo-3-chloro-2-((4-methoxybenzyl)oxy)phenyl)-1,3-dioxolane (Intermediate 9-1) (2.4 g, 85%) as a yellow oil. MS (ESI) calculated for C₁₇H₁₆BrClO₄: 297.99 m/z, found 298.95 [M+H]⁺.

### Example 10: 5-(2-(2-aminopyridin-3-yl)-5-phenyl-3H-imidazo[4,5-b]pyridin-3-yl)-N-(4-formyl-3-hydroxybenzyl)-2,3-dihydro-1H-indene-1-carboxamide

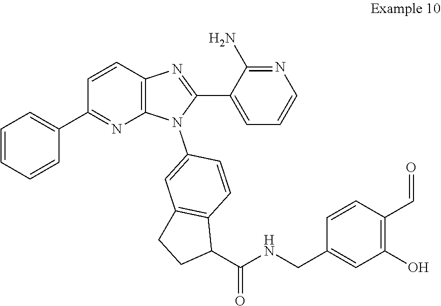 [SMILES: Nc1ncccc1-c1nc2ccc(-c3ccccc3)nc2n1-c1ccc2c(c1)CCC2C(=O)NCc1ccc(C=O)c(O)c1]

### Synthetic Route:

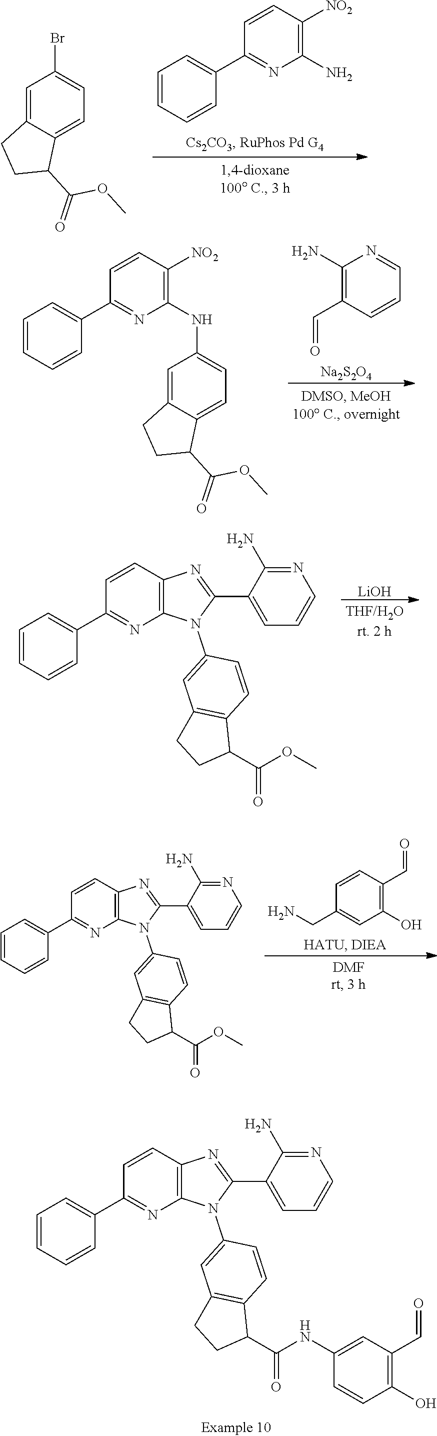

### Step 1: Synthesis of methyl 5-[(3-nitro-6-phenylpyridin-2-yl)amino]-2,3-dihydro-1H-indene-1-carboxylate

To a stirred solution of methyl 5-bromo-2,3-dihydro-1H-indene-1-carboxylate (150 mg, 0.59 mmol, 1 equiv), 3-nitro-6-phenylpyridin-2-amine (127 mg, 0.59 mmol, 1 equiv) and RuPhos Pd G4 (CAS 1599466-85-9) (50 mg, 0.059 mmol, 0.1 equiv) in anhydrous 1,4-dioxane (2 mL) was added cesium carbonate (383 mg, 1.18 mmol, 2 equiv). The resulting suspension was stirred for 3 h at 100° C. under N₂. The mixture was cooled to room temperature followed by addition of ethyl acetate (10 mL) and water (10 mL). The layers were separated, and the aqueous layer was extracted with ethyl acetate (10 mL×2). The combined organic layers were washed with brine (30 mL×3), dried over anhydrous sodium sulfate, filtered and concentrated. The residue was purified on a silica gel column using a gradient of 0-100% ethyl acetate in petroleum ether to afford methyl 5-[(3-nitro-6-phenylpyridin-2-yl)amino]-2,3-dihydro-1H-indene-1-carboxylate (120 mg, 47%) as a red solid. MS (ESI) calculated for C₂₂H₁₉N₃O₄: 389.14 m/z, found 390.15 [M+H]⁺.

### Step 2: Synthesis of methyl 5-[2-(2-aminopyridin-3-yl)-5-phenylimidazo[4,5-b]pyridin-3-yl]-2,3-dihydro-1H-indene-1-carboxylate

To a stirred solution of methyl 5-[(3-nitro-6-phenylpyridin-2-yl)amino]-2,3-dihydro-1H-indene-1-carboxylate (200 mg, 0.51 mmol, 1 equiv), 2-aminopyridine-3-carbaldehyde (82 mg, 0.67 mmol, 1.3 equiv) and sodium dithionite (179 mg, 1.03 mmol, 2 equiv) in DMSO (10 mL) was added methanol (1.7 mL). The resulting mixture was stirred at 100° C. for 18 h then cooled to room temperature and purified by reverse-phase flash column chromatography on C18 silica gel using a 20 to 95% gradient of acetonitrile in water (+0.05% TFA) with a 10 minute hold at 70% acetonitrile. This afforded methyl 5-[2-(2-aminopyridin-3-yl)-5-phenylimidazo[4,5-b]pyridin-3-yl]-2,3-dihydro-1H-indene-1-carboxylate (90 mg, 34%) as a brown/yellow solid. MS (ESI) calculated for C₂₈H₂₃N₅O₂: 461.19 m/z, found 462.05 [M+H]⁺.

### Step 3: Synthesis of 5-[2-(2-aminopyridin-3-yl)-5-phenylimidazo[4,5-b]pyridin-3-yl]-2,3-dihydro-1H-indene-1-carboxylic acid

To a stirred solution of methyl 5-[2-(2-aminopyridin-3-yl)-5-phenylimidazo[4,5-b]pyridin-3-yl]-2,3-dihydro-1H-indene-1-carboxylate (90 mg, 0.20 mmol, 1 equiv) in tetrahydrofuran (5 mL) and methanol (1 mL) was added a solution of lithium hydroxide (28 mg, 1.2 mmol, 6 equiv) in water (1 mL). The reaction mixture was stirred at 25° C. for 2 h and concentrated. The crude residue was purified by reverse-phase column chromatography on a Xselect CSH OBD column (30×150 mm, 5 um) using a 5-30% gradient of acetonitrile in water (+0.05% ammonium bicarbonate) to afford 5-[2-(2-aminopyridin-3-yl)-5-phenylimidazo[4,5-b]pyridin-3-yl]-2,3-dihydro-1H-indene-1-carboxylic acid (85 mg, 89%) as a yellow solid. MS (ESI) calculated for C₂₇H₂₁N₅O₂: 447.17 m/z, found 448.20 [M+H]⁺.

### Step 4: Synthesis of 5-[2-(2-aminopyridin-3-yl)-5-phenylimidazo[4,5-b]pyridin-3-yl]-N-[(4-formyl-3-hydroxyphenyl)methyl]-2,3-dihydro-1H-indene-1-carboxamide (Example 10)

To a stirred solution of 5-[2-(2-aminopyridin-3-yl)-5-phenylimidazo[4,5-b]pyridin-3-yl]-2,3-dihydro-1H-indene-1-carboxylic acid (100 mg, 0.22 mmol, 1 equiv), 4-(aminomethyl)-2-hydroxybenzaldehyde (68 mg, 0.45 mmol, 2 equiv) and HATU (127 mg, 0.34 mmol, 1.5 equiv) in anhydrous N,N-dimethylformamide (2 mL) was added N,N-diisopropylethylamine (0.27 mL, 1.6 mmol, 7 equiv) and the resulting solution was stirred at room temperature for 3 h. The mixture was purified by reverse-phase chromatography on a Xselect CSH OBD column (30×150 mm, 5 um) using a 13 to 47% gradient of acetonitrile in water (+0.05% TFA) to afford 5-[2-(2-aminopyridin-3-yl)-5-phenylimidazo[4,5-b]pyridin-3-yl]-N-[(4-formyl-3-hydroxyphenyl)methyl]-2,3-dihydro-1H-indene-1-carboxamide (Example 10) (19.5 mg, 12%) as a yellow solid. MS (ESI) calculated for C₃₅H₂₈N₆O₃: 580.22 m/z, found 581.25 [M+H]⁺. ¹H NMR (400 MHz, DMSO-d₆) δ 10.20 (s, 1H), 8.30-8.35 (m, 1H), 8.02-8.08 (m, 4H), 7.73-7.75 (m, 1H), 7.58-7.72 (m, 1H), 7.50-7.54 (m, 2H), 7.38-7.50 (m, 3H), 7.26-7.38 (m, 1H), 6.90-6.98 (m, 1H), 6.84-6.90 (m, 1H), 6.73-6.80 (m, 1H), 4.36 (s, 2H), 4.02-4.12 (m, 1H), 3.03-3.12 (m, 1H), 2.87-3.01 (m, 1H), 2.28-2.41 (m, 2H).

### Example 11: N-[(1S)-5-[2-(2-aminopyridin-3-yl)-5-phenylimidazo[4,5-b]pyridin-3-yl]-2,3-dihydro-1H-inden-1-yl]-3-chloro-5-formyl-4-hydroxybenzamide

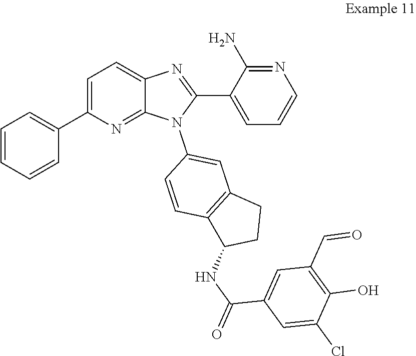 [SMILES: Nc1ncccc1-c1nc2ccc(-c3ccccc3)nc2n1-c1ccc2c(c1)CC[C@@H]2NC(=O)c1cc(Cl)c(O)c(C=O)c1]

### Synthetic Route:

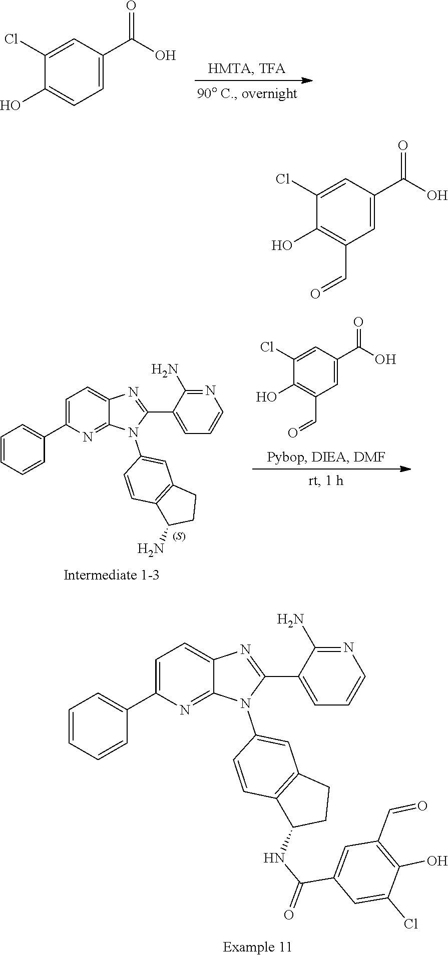

### Step 1: Synthesis of 3-chloro-5-formyl-4-hydroxybenzoic acid

To a stirred solution of 3-chloro-4-hydroxybenzoic acid (1 g, 5.8 mmol, 1 equiv) in TFA (5 mL) was added dropwise a solution of 1,3,5,7-tetraazaadamantane (1.22 g, 8.7 mmol, 1.5 equiv) in TFA (5 mL) over 15-20 min. The reaction mixture was then refluxed at 90° C. overnight. After cooling to room temperature, 30 mL of water was added, and the reaction mixture was acidified with 4 N aqueous HCl solution. Precipitation occurred gradually with acidification. The resulting mixture was stirred at room temperature until the completion of precipitation. The yellow precipitate was then filtered off and dried. Recrystallization from water gave 3-chloro-5-formyl-4-hydroxybenzoic acid as a yellow solid (1.1 g, 95%). MS (ESI) calculated for C₈H₅ClO₄: 199.99 m/z, found 198.95 [M−H]⁻.

### Step 2: Synthesis of N-[(1S)-5-[2-(2-aminopyridin-3-yl)-5-phenylimidazo[4,5-b]pyridin-3-yl]-2,3-dihydro-1H-inden-1-yl]-3-chloro-5-formyl-4-hydroxybenzamide (Example 11)

To a solution of 3-{3-[(1S)-1-amino-2,3-dihydro-1H-inden-5-yl]-5-phenylimidazo[4,5-b]pyridin-2-yl}pyridin-2-amine (Intermediate 1-3) (240 mg, 0.60 mmol, 1.5 equiv) in N,N-dimethylformamide (3 mL) was added N,N-diisopropylethylamine (258 mg, 2.0 mmol, 5 equiv), Pybop (311 mg, 0.60 mmol, 1.5 equiv), and 3-chloro-5-formyl-4-hydroxybenzoic acid (80 mg, 0.40 mmol, 1 equiv). The resulting mixture was stirred at room temperature for 1 h. The crude product was purified by Preparative HPLC on a Xselect CSH OBD Column (30×150 mm, 5 um) using a 23 to 50% gradient of acetonitrile in water (+0.05% TFA) to afford N-[(1S)-5-[2-(2-aminopyridin-3-yl)-5-phenylimidazo[4,5-b]pyridin-3-yl]-2,3-dihydro-1H-inden-1-yl]-3-chloro-5-formyl-4-hydroxybenzamide (Example 11) (5.7 mg, 2.3%) as an off-white solid. MS (ESI) calculated for C₃₄H₂₅ClN₆O₃: 600.17 m/z, found 601.15 [M+H]⁺. ¹H-NMR (400 MHz, DMSO-d₆) δ (ppm): 10.17 (s, 1H), 8.29-8.32 (m, 3H), 8.00-8.05 (m, 4H), 7.70-8.72 (m, 1H), 7.35-7.50 (m, 6H), 6.75-6.78 (m, 1H), 5.59-5.63 (m, 1H), 3.03-3.05 (m, 1H), 2.89-2.93 (m, 1H), 2.51 (s, 1H), 2.06-2.11 (m, 1H).

### Example 12: N-[(1S)-5-[2-(2-aminopyridin-3-yl)-5-phenylimidazo[4,5-b]pyridin-3-yl]-2,3-dihydro-1H-inden-1-yl]-1-(6-formyl-5-hydroxypyridin-3-yl)cyclopropane-1-carboxamide

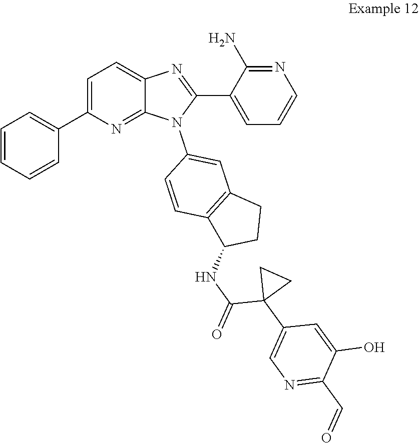 [SMILES: Nc1ncccc1-c1nc2ccc(-c3ccccc3)nc2n1-c1ccc2c(c1)CC[C@@H]2NC(=O)C1(c2cnc(C=O)c(O)c2)CC1]

### Synthetic Route:

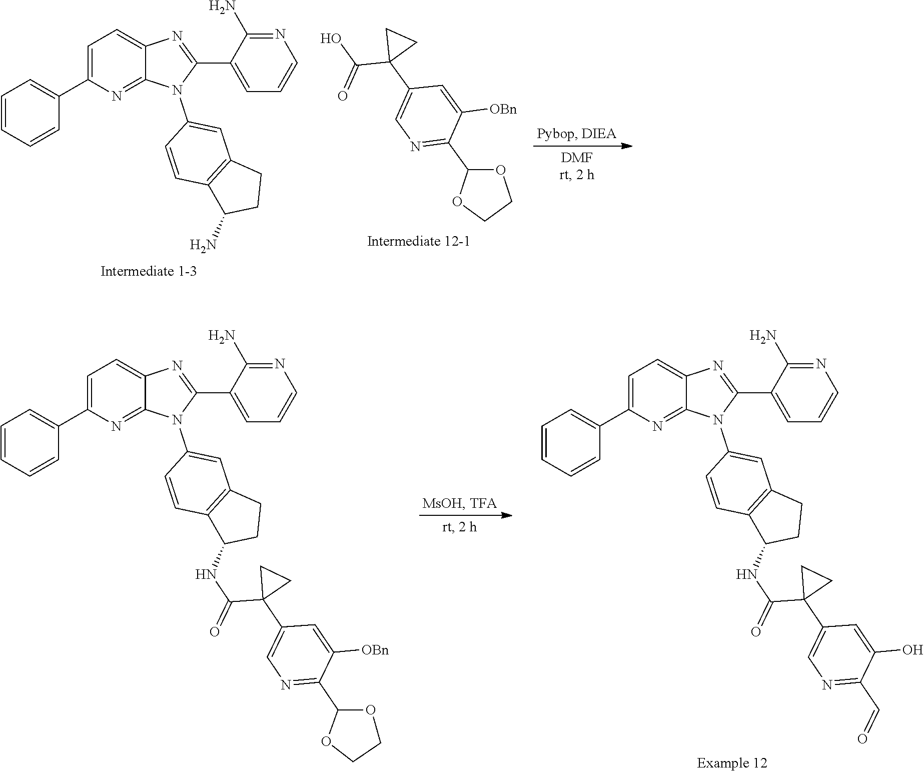 [SMILES: Nc1ncccc1-c1nc2ccc(-c3ccccc3)nc2n1-c1ccc2c(c1)CC[C@@H]2N ; O=C(O)C1(c2cnc(C3OCCO3)c(OCc3ccccc3)c2)CC1 ; Nc1ncccc1-c1nc2ccc(-c3ccccc3)nc2n1-c1ccc2c(c1)CC[C@@H]2NC(=O)C1(c2cnc(C3OCCO3)c(OCc3ccccc3)c2)CC1 ; Nc1ncccc1-c1nc2ccc(-c3ccccc3)nc2n1-c1ccc2c(c1)CC[C@@H]2NC(=O)C1(c2cnc(C=O)c(O)c2)CC1]

### Step 2: Synthesis of N-[(1S)-5-[2-(2-aminopyridin-3-yl)-5-phenylimidazo[4,5-b]pyridin-3-yl]-2,3-dihydro-1H-inden-1-yl]-1-(6-formyl-5-hydroxypyridin-3-yl)cyclopropane-1-carboxamide (Example 12)

A mixture of N-[(1S)-5-[2-(2-aminopyridin-3-yl)-5-phenylimidazo[4,5-b]pyridin-3-yl]-2,3-dihydro-1H-inden-1-yl]-1-[5-(benzyloxy)-6-(1,3-dioxolan-2-yl)pyridin-3-yl]cyclopropane-1-carboxamide (118 mg, 0.16 mmol, 1 equiv), TFA (1.5 mL) and methanesulfonic acid (0.5 mL) was stirred at room temperature for 2 h. The mixture was neutralized aqueous sodium bicarbonate and extracted with ethyl acetate (3×30 mL). The combined organic layers were dried over anhydrous sodium sulfate, filtered and concentrated in vacuo. The resulting residue was purified by Preparative HPLC on a XBridge Prep OBD C18 column (30×150 mm, 5 μm) using a 17 to 42% gradient of acetonitrile in water to afford N-[(1S)-5-[2-(2-aminopyridin-3-yl)-5-phenylimidazo[4,5-b]pyridin-3-yl]-2,3-dihydro-1H-inden-1-yl]-1-(6-formyl-5-hydroxypyridin-3-yl)cyclopropane-1-carboxamide (Example 12) (11.2 mg, 11%) as a light yellow solid. MS (ESI) calculated for C₃₆H₂₉N₇O₃: 607.67 m/z, found 608.25 [M+H]⁺. ¹H-NMR (400 MHz, DMSO-d₆) δ (ppm): 10.50 (s, 1H), 8.11-8.25 (m, 2H), 7.71-7.99 (m, 5H), 7.01-7.49 (m, 8H), 6.32-6.41 (m, 1H), 5.40 (s, 1H), 2.70-2.89 (m, 2H), 2.25-2.38 (m, 1H), 1.79-1.92 (m, 1H), 1.20-1.41 (m, 2H), 0.92-1.15 (m, 2H).

### Intermediate 12-1: 1-(5-(benzyloxy)-6-(1,3-dioxolan-2-yl)pyridin-3-yl)cyclopropane-1-carboxylic acid

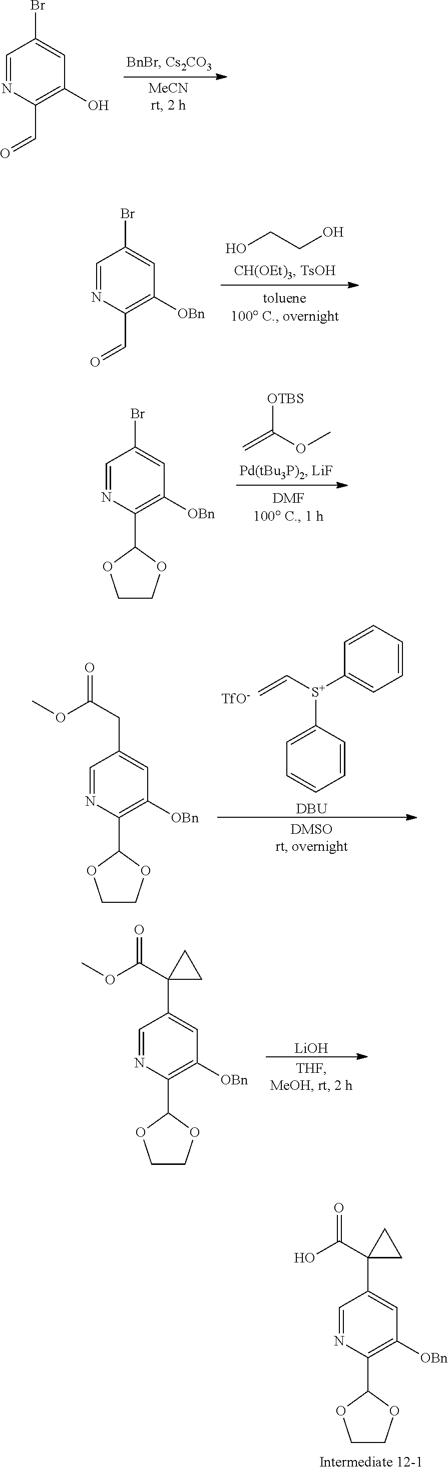

### Step 1: Synthesis of 3-(benzyloxy)-5-bromopicolinaldehyde

To a solution of 5-bromo-3-hydroxypicolinaldehyde (4.01 g, 20.0 mmol, 1 equiv) and benzyl bromide (4.10 g, 24.0 mmol, 1.2 equiv) in acetonitrile (40 mL) was added cesium carbonate (19.5 g, 60.0 mmol, 3 equiv). The resulting mixture was stirred for 18 h at room temperature and partially concentrated in vacuo. The mixture was then cooled to 0° C. and quenched by addition of saturated aqueous ammonium chloride (20 mL). The resulting mixture was extracted with ethyl acetate (30 mL×3). The combined organic layers were washed with water (30 mL×3), dried over anhydrous sodium sulfate, filtered and concentrated in vacuo. The resulting residue was purified by silica gel column chromatography using a 0-10% gradient of ethyl acetate in petroleum ether to provide 3-(benzyloxy)-5-bromopicolinaldehyde (5.0 g, 86%) as a white solid. MS (ESI) calculated For C₁₃H₁₀BrNO₂: 290.99, found 291.10 [M+H]⁺.

### Step 2: Synthesis of 3-(benzyloxy)-5-bromo-2-(1,3-dioxolan-2-yl)pyridine

To a solution of 3-(benzyloxy)-5-bromopicolinaldehyde (5.0 g, 17 mmol, 1 equiv) in toluene (100 mL) was added p-toluenesulfonic acid (0.29 g, 1.7 mmol, 0.1 equiv), ethylene glycol (5.3 g, 86 mmol, 5 equiv) and triethyl orthoformate (7.6 g, 51 mmol, 3 equiv). The resulting solution was stirred at room temperature for 10 min and then at 90° C. for 18 h. The mixture was cooled to 0° C. and quenched by the addition of saturated aqueous ammonium chloride (80 mL). The resulting mixture was extracted with ethyl acetate (100 mL×3). The combined organic layers were washed with water (100 mL×3), dried over anhydrous sodium sulfate, filtered and concentrated in vacuo. The resulting residue was purified by silica gel column chromatography using a 0-10% gradient of ethyl acetate in petroleum ether to provide 3-(benzyloxy)-5-bromo-2-(1,3-dioxolan-2-yl)pyridine (3.0 g, 52% yield) as a light-yellow oil. MS (ESI) calculated for C₁₅H₁₄BrNO₃: 335.02, found 336.10 [M+H]⁺.

### Step 3: Synthesis of methyl 2-(5-(benzyloxy)-6-(1,3-dioxolan-2-yl)pyridin-3-yl)acetate

To a mixture of 3-(benzyloxy)-5-bromo-2-(1,3-dioxolan-2-yl)pyridine (1.70 g, 5.08 mmol, 1 equiv), tert-butyl[(1-methoxyethenyl)oxy]dimethylsilane (3.82 g, 20.3 mmol, 4.0 equiv), Pd(t-Bu₃P)₂ (0.26 g, 0.51 mmol, 0.1 equiv) and lithium fluoride (0.26 g, 10.2 mmol, 2.0 equiv) was added N,N-dimethylformamide (10 mL) under N₂. The mixture was stirred at 100° C. for 1 h and quenched by addition of water (50 mL). The aqueous layer was extracted with ethyl acetate (50 mL), then concentrated under reduced pressure. The resulting residue was purified by silica gel column chromatography using a gradient of ethyl acetate in petroleum ether to afford methyl 2-(5-(benzyloxy)-6-(1,3-dioxolan-2-yl)pyridin-3-yl)acetate (1.0 g, 60%) as a yellow oil. MS (ESI) calculated for C₁₈H₁₉NO₅; 329.13 m/z, found 330.10[M+H]⁺.

### Step 4: Synthesis of methyl 1-(5-(benzyloxy)-6-(1,3-dioxolan-2-yl)pyridin-3-yl)cyclopropane-1-carboxylate

To a mixture of methyl 2-(5-(benzyloxy)-6-(1,3-dioxolan-2-yl)pyridin-3-yl)acetate (1.0 g, 3 mmol, 1 equiv), ethenyldiphenylsulfanium triflate (0.97 g, 4.55 mmol, 1.5 equiv) and 1,8-diazabicyclo(5.4.0)undec-7-ene (1.37 g, 9.02 mmol, 3.0 equiv) was added DMSO (15 mL). The mixture was stirred at room temperature overnight then quenched by addition of water (50 mL). The aqueous layer was extracted with ethyl acetate (50 mL), then concentrated under reduced pressure. The residue was purified by silica gel column chromatography, using a gradient of ethyl acetate in petroleum ether to afford methyl 1-(5-(benzyloxy)-6-(1,3-dioxolan-2-yl)pyridin-3-yl)cyclopropane-1-carboxylate (800 mg, 74%) as a yellow oil. MS (ESI) calculated for C₂₀H₂₁NO₅: 355.14 m/z, found 356.10 [M+H]⁺.

### Step 5: Synthesis of 1-(5-(benzyloxy)-6-(1,3-dioxolan-2-yl)pyridin-3-yl)cyclopropane-1-carboxylic acid (Intermediate 12-1)

To a solution of methyl 1-(5-(benzyloxy)-6-(1,3-dioxolan-2-yl)pyridin-3-yl)cyclopropane-1-carboxylate (800 mg, 2.25 mmol, 1 equiv) in tetrahydrofuran (10 mL) and methanol (10 mL) was added lithium hydroxide (3.5 mL, 2 M in H₂O). The resulting mixture was stirred at room temperature for 2 h. The mixture was concentrated in vacuo and purified by reverse-phase flash column chromatography on C18 silica gel using a 20 to 95% gradient of acetonitrile in water (+0.05% ammonium bicarbonate) with a 40 minute hold at 70% acetonitrile to afford 1-(5-(benzyloxy)-6-(1,3-dioxolan-2-yl)pyridin-3-yl)cyclopropane-1-carboxylic acid (Intermediate 12-1) (650 mg, 78%). MS (ESI) calculated for Cl₉H₁₉NO₅: 341.13 m/z, found 342.13 [M+H]⁺.

### Example 13: N-[(1S)-5-[2-(2-aminopyridin-3-yl)-5-methylimidazo[4,5-b]pyridin-3-yl]-2,3-dihydro-1H-inden-1-yl]-3-formyl-4-hydroxybenzamide

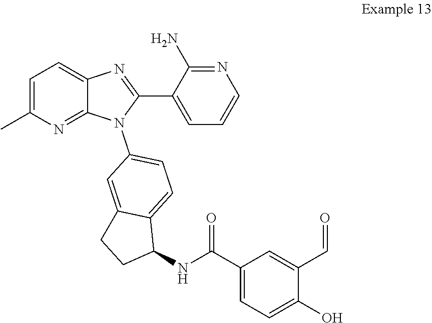 [SMILES: Cc1ccc2nc(-c3cccnc3N)n(-c3ccc4c(c3)CC[C@@H]4NC(=O)c3ccc(O)c(C=O)c3)c2n1]

Example 13 was prepared in a manner analogous to Example 12 using Intermediate 13-2 in place of Intermediate 1-3 and Intermediate 5-1 in pace of Intermediate 12-1. MS (ESI) calculated for C₂₉H₂₄N₆O₃: 504.19 m/z, found 505.20 [M+H]⁺. ¹H-NMR (400 MHz, DMSO-d₆) δ (ppm): 10.20-10.40 (m, 1H), 8.85-8.90 (m, 1H), 8.10-8.40 (m, 3H), 7.90-8.10 (m, 2H), 7.40-7.50 (m, 2H), 7.25-7.50 (m, 2H), 7.10-7.20 (m, 1H), 6.90-7.00 (m, 1H), 6.40-6.50 (m, 1H), 5.50-5.70 (m, 1H), 2.80-3.10 (m, 2H), 2.50 (s, 4H), 2.00-2.10 (m, 1H).

### Intermediate 13-2: (S)-3-(3-(1-amino-2,3-dihydro-1H-inden-5-yl)-5-methyl-3H-imidazo[4,5-b]pyridin-2-yl)pyridin-2-amine

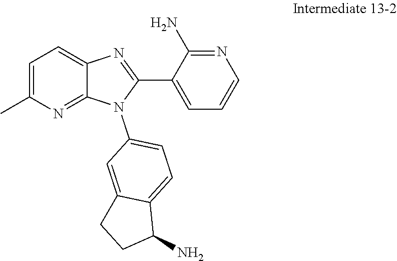 [SMILES: Cc1ccc2nc(-c3cccnc3N)n(-c3ccc4c(c3)CC[C@@H]4N)c2n1]

### Synthetic Route:

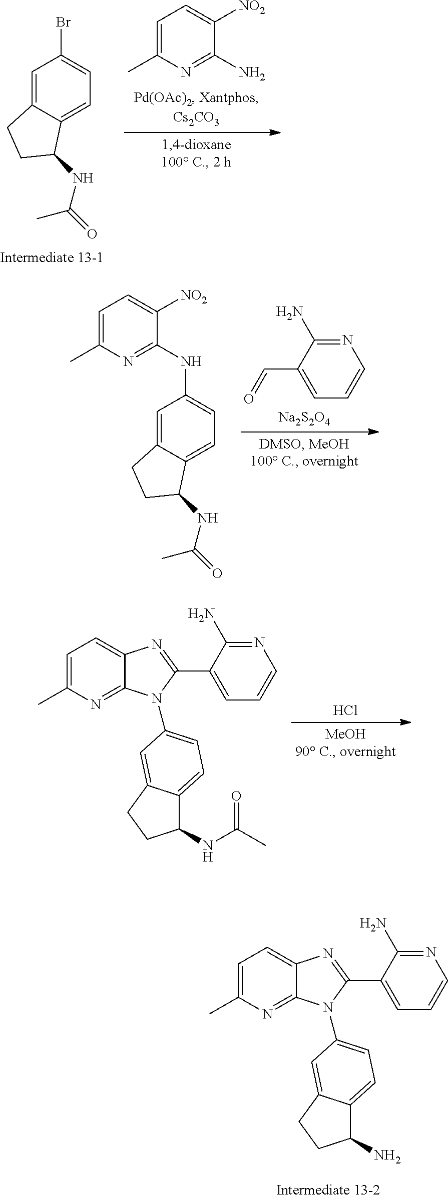

### Step 1: Synthesis of N-[(1S)-5-[(6-methyl-3-nitropyridin-2-yl)amino]-2,3-dihydro-1H-inden-1-yl]acetamide

To a solution of N-[(1S)-5-bromo-2,3-dihydro-1H-inden-1-yl]acetamide (Intermediate 13-1) (600 mg, 2.36 mmol, 1 equiv), 6-methyl-3-nitropyridin-2-amine (434 mg, 2.8 mmol, 1.2 equiv) and XantPhos (CAS: 161265-03-8) (273 mg, 0.47 mmol, 0.2 equiv) in 1,4-dioxane (10 mL) were added cesium carbonate (2.3 g 7.1 mmol, 3 equiv) and Pd(OAc)₂ (53 mg, 0.24 mmol, 0.1 equiv). The resulting mixture was stirred at 100° C. for 2 h under nitrogen atmosphere. The resulting mixture was concentrated under reduced pressure. The resulting residue was purified by reverse-phase flash chromatography on C18 silica gel using a 10 to 50% gradient of acetonitrile in water (+0.05% ammonium bicarbonate) to afford N-[(1S)-5-[(6-methyl-3-nitropyridin-2-yl)amino]-2,3-dihydro-1H-inden-1-yl]acetamide (200 mg, 26%) as a brown solid. MS (ESI) calculated for C₁₇H₁₈N₄O₃: 326.14 m/z, found 327.05 [M+H]⁺.

### Step 2: Synthesis of N-[(1S)-5-[2-(2-aminopyridin-3-yl)-5-methylimidazo[4,5-b]pyridin-3-yl]-2,3-dihydro-1H-inden-1-yl]acetamide

To a solution of N-[(1S)-5-[(6-methyl-3-nitropyridin-2-yl)amino]-2,3-dihydro-H-inden-1-yl]formamide (190 mg, 0.61 mmol, 1 equiv) in DMSO (5 mL) and methanol (1 mL) were added 2-aminopyridine-3-carbaldehyde (78 mg, 0.64 mmol, 1.1 equiv) and sodium dithionite (223 mg, 1.3 mmol, 2.2 equiv). The resulting mixture was stirred overnight at 100° C. under a nitrogen atmosphere. The resulting mixture was concentrated under reduced pressure and the residue purified by reverse-phase flash chromatography on C18 silica gel using a 10 to 50% gradient of acetonitrile in water (+0.05% ammonium bicarbonate) to afford N-[(1S)-5-[2-(2-aminopyridin-3-yl)-5-methylimidazo[4,5-b]pyridin-3-yl]-2,3-dihydro-1H-inden-1-yl]acetamide (135 mg, 46%) as a yellow solid. MS (ESI) calculated for C₂₃H₂₂N₆O: 398.19 m/z, found 399.25 [M+H]⁺.

### Step 3: Synthesis of 3-{3-[(1S)-1-amino-2,3-dihydro-1H-inden-5-yl]-5-methylimidazo[4,5-b]pyridin-2-yl}pyridin-2-amine (Intermediate 13-2)

N-[(1S)-5-[2-(2-aminopyridin-3-yl)-5-methylimidazo[4,5-b]pyridin-3-yl]-2,3-dihydro-1H-inden-1-yl]acetamide (135 mg, 0.34 mmol, 1 equiv) was dissolved methanol (10 mL). HCl (10 mL, concentrated) was added, and the resulting mixture was stirred at 90° C. overnight. The resulting mixture was cooled to room temperature and concentrated under reduced pressure to afford 3-{3-[(1S)-1-amino-2,3-dihydro-1H-inden-5-yl]-5-methylimidazo[4,5-b]pyridin-2-yl}pyridin-2-amine (Intermediate 13-2) (100 mg, 75%) as a yellow solid. MS (ESI) calculated for C₂₁H₂₀N₆: 356.17 m/z, found 357.15 [M+H]⁺. The crude product was used in subsequent transformations directly without further purification.

### Intermediate 13-1: (S)—N-(5-bromo-2,3-dihydro-1H-inden-1-yl)acetamide

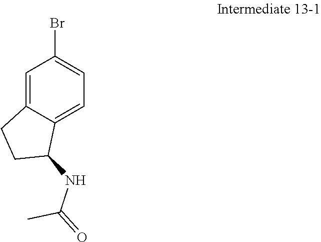

### Synthetic Route:

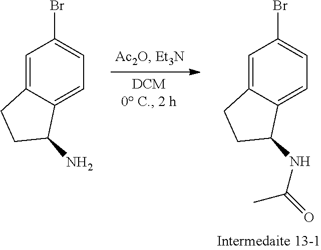

To a mixture of (S)-5-bromo-2,3-dihydro-1H-inden-1-amine (74 g, 350 mmol, 1 equiv) and triethylamine (106 g, 1.05 mol, 3 equiv) in dichloromethane (1.5 L) was added acetic anhydride (55.2 g, 526 mmol, 1.5 equiv) at 0° C. and the mixture was stirred at 0° C. for 2 h. The reaction mixture was quenched by addition of water (500 mL) and extracted with ethyl acetate (3×500 mL). The combined organic layers were dried over anhydrous sodium sulfate and concentrated under reduced pressure. The crude product was re-crystallized from petroleum ether to afford (S)—N-(5-bromo-2,3-dihydro-1H-inden-1-yl)acetamide (Intermediate 13-1) (90 g, 83% yield) as a white solid. MS (ESI) calculated for C₁₁H₁₂BrNO: 253.01, found 254.00 [M+H]⁺, 256.00 [M+H+2]⁺.

### Example 14: N-[(1S)-5-[2-(2-aminopyridin-3-yl)-5-phenylimidazo[4,5-b]pyridin-3-yl]-2,3-dihydro-1H-inden-1-yl]-4-acetamido-3-formylbenzamide

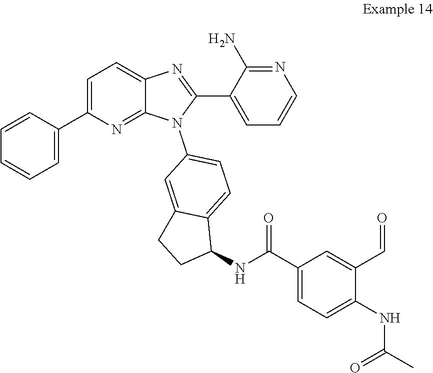 [SMILES: CC(=O)Nc1ccc(C(=O)N[C@H]2CCc3cc(-n4c(-c5cccnc5N)nc5ccc(-c6ccccc6)nc54)ccc32)cc1C=O]

### Synthetic Route:

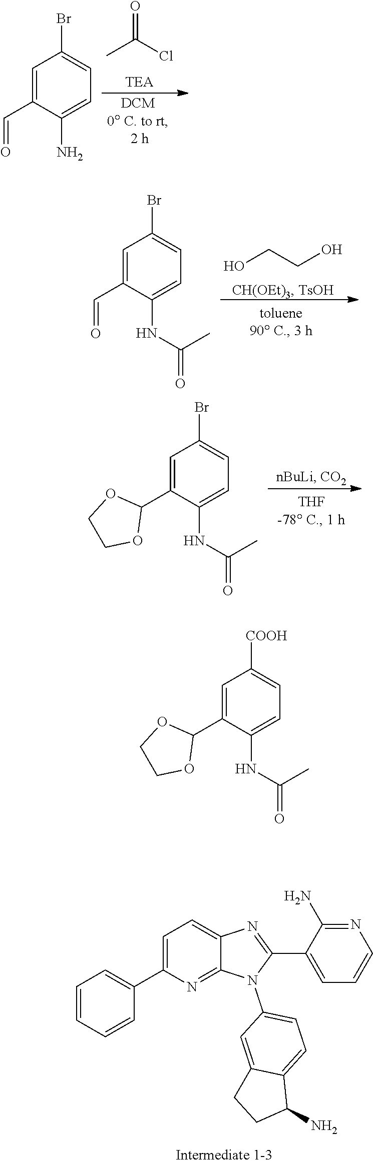

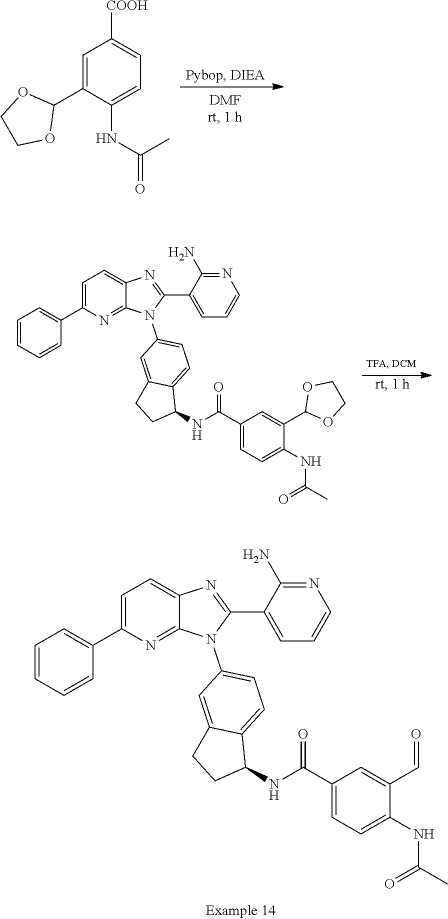 [SMILES: CC(=O)Nc1ccc(C(=O)O)cc1C1OCCO1 ; CC(=O)Nc1ccc(C(=O)N[C@H]2CCc3cc(-n4c(-c5cccnc5N)nc5ccc(-c6ccccc6)nc54)ccc32)cc1C1OCCO1 ; CC(=O)Nc1ccc(C(=O)N[C@H]2CCc3cc(-n4c(-c5cccnc5N)nc5ccc(-c6ccccc6)nc54)ccc32)cc1C=O]

### Step 1: Synthesis of N-(4-bromo-2-formylphenyl)acetamide

To a cooled (0° C.) solution of 2-amino-5-bromobenzaldehyde (3 g, 15 mmol, 1 equiv) in dichloromethane (100 mL) was added triethylamine (6.1 g, 60 mmol, 4 equiv) followed by acetyl chloride (4.7 g, 40 mmol, 4 equiv). The resulting mixture was allowed to warm to room temperature and stirred for 2 h. The reaction was quenched with H₂O (100 mL) and extracted with dichloromethane (3×50 mL). The organic layers were combined, dried over anhydrous sodium sulfate, filtered and concentrated in vacuo. The resulting residue was purified by silica gel chromatography using a 0-30% gradient of ethyl acetate in petroleum ether) to afford N-(4-bromo-2-formylphenyl)acetamide (1.4 g, 39%) as a white solid. MS (ESI) calculated for C₉H₈BrNO₂: 240.97 m/z, found 242.00 [M+H]⁺.

### Step 2: Synthesis of N-[4-bromo-2-(1,3-dioxolan-2-yl)phenyl]acetamide

To a solution of N-(4-bromo-2-formylphenyl)acetamide (800 mg, 3.3 mmol, 1 equiv) in toluene (30 mL) was added ethylene glycol (1.03 g, 16.5 mmol, 5 equiv), p-toluenesulfonic acid (285 mg, 1.7 mmol, 0.5 equiv) and (diethoxymethoxy)ethane (1.47 g, 9.9 mmol, 3 equiv). The resulting mixture was stirred at 90° C. for 3 h. The reaction mixture was concentrated in vacuo and the resulting residue was purified by silica gel column chromatography using a 0-10% gradient of ethyl acetate in petroleum ether to afford N-[4-bromo-2-(1,3-dioxolan-2-yl)phenyl]acetamide (700 mg, 74%) as a white solid. MS (ESI) calculated for C₁₁H₁₂BrNO₃: 285.00 m/z, found 285.95 [M+H]⁺.

### Step 3: Synthesis of 3-(1,3-dioxolan-2-yl)-4-acetamidobenzoic acid

To a cooled (−78° C.) solution of N-[4-bromo-2-(1,3-dioxolan-2-yl)phenyl]acetamide (500 mg, 1.75 mmol, 1 equiv) in tetrahydrofuran (10 mL) was added dropwise n-butyllithium solution (2.5 M in hexane, 1.75 mL, 4.4 mmol, 2.5 equiv) under N₂ atmosphere. The reaction mixture was stirred at −78° C. for 30 mins. Then CO₂ was bubbled through the reaction mixture for 10 minutes and stirring continued for 1 h under CO₂ atmosphere. The reaction was quenched with saturated aqueous ammonium chloride (10 mL) and the mixture was extracted with ether/ethyl acetate (2×15 mL). The combined organic extracts were washed with brine (10 mL), dried over anhydrous sodium sulfate, and concentrated to afford 3-(1,3-dioxolan-2-yl)-4-acetamidobenzoic acid (150 mg, 34%) as a light yellow solid, which was used in the next step directly without further purification. MS (ESI) calculated for C₁₂H₁₃NO₅: 251.08 m/z, found 252.05 [M+H]⁺.

### Step 4: Synthesis of N-[(1S)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-acetamidobenzamide

To a solution of 3-{3-[(1S)-1-amino-2,3-dihydro-1H-inden-5-yl]-5-phenylimidazo[4,5-b]pyridin-2-yl}pyridin-2-amine (Intermediate 1-3) (100 mg, 0.24 mmol, 1 equiv) in N,N-dimethylformamide (2 mL) was added N,N-diisopropylethylamine (93 mg, 0.72 mmol, 3 equiv), Pybop (187 mg, 0.36 mmol, 1.5 equiv) and 3-(1,3-dioxolan-2-yl)-4-acetamidobenzoic acid (90 mg, 0.36 mmol, 1.5 equiv). The resulting mixture was stirred at room temperature for 1 h. The resulting mixture was purified by reverse-phase flash column chromatography on C18 silica gel using a 20-95% gradient of acetonitrile in water (+0.05% ammonium bicarbonate) with a 10 minute hold at 70% acetonitrile to afford N-[(1S)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-acetamidobenzamide (70 mg, 45%) as a yellow solid. MS (ESI) calculated for C₃₈H₃₃N₇O₄: 651.26 m/z, found 652.25 [M+H]⁺.

### Step 5: Synthesis of N-[(1S)-5-[2-(2-aminopyridin-3-yl)-5-phenylimidazo[4,5-b]pyridin-3-yl]-2,3-dihydro-1H-inden-1-yl]-4-acetamido-3-formylbenzamide (Example 14)

To a solution of N-[(1S)-5-[2-(2-aminopyridin-3-yl)-5-phenylimidazo[4,5-b]pyridin-3-yl]-2,3-dihydro-1H-inden-1-yl]-3-(1,3-dioxolan-2-yl)-4-acetamidobenzamide (100 mg, 0.15 mmol, 1 equiv) in dichloromethane (5 mL) was added TFA (2 mL). The resulting mixture was stirred at room temperature for 1 h and concentrated in vacuo. The resulting residue was purified by Preparative HPLC on a XBridge Prep OBD C18 column (30×150 mm, 5 μm) using a 32-62% gradient of acetonitrile in water (+10 mmol/L ammonium bicarbonate) to afford N-[(1S)-5-[2-(2-aminopyridin-3-yl)-5-phenylimidazo[4,5-b]pyridin-3-yl]-2,3-dihydro-1H-inden-1-yl]-4-acetamido-3-formylbenzamide (Example 14) (11.5 mg, 12%) as an off-white solid. MS (ESI) calculated for C₃₆H₂₉N₇O₃: 607.23 m/z, found 608.25 [M+H]⁺. ¹H-NMR (400 MHz, DMSO-d₆) δ (ppm): 9.98 (s, 1H), 8.43 (s, 1H), 8.17-8.26 (m, 3H), 7.95-8.02 (m, 4H), 7.45-7.48 (m, 2H), 7.37-7.41 (m, 3H), 7.26-7.32 (m, 2H), 6.44-6.48 (m, 1H), 5.62-5.66 (m, 1H), 3.00-3.02 (m, 1H), 2.88-2.92 (m, 1H), 2.51-2.56 (m, 1H), 2.12-2.18 (m, 3H), 2.04-2.10 (m, 1H).

### Example 15: N-[(1S)-5-[2-(2-aminopyridin-3-yl)imidazo[4,5-b]pyridin-3-yl]-2,3-dihydro-1H-inden-1-yl]-3-formyl-4-hydroxybenzamide

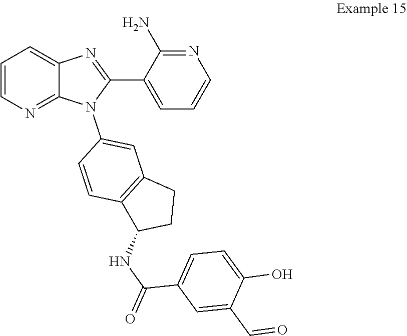 [SMILES: Nc1ncccc1-c1nc2cccnc2n1-c1ccc2c(c1)CC[C@@H]2NC(=O)c1ccc(O)c(C=O)c1]

Example 15 was synthesized in a manner analogous to Example 12 using Intermediate 15-1 in place Intermediate 1-3 of and Intermediate 5-1 in pace of Intermediate 12-1. MS (ESI) calculated for C₂₈H₂₂N₆O₃: 490.18 m/z, found 491.20 [M+H]⁺. ¹H-NMR (400 MHz, DMSO-d₆) δ (ppm): 10.30 (s, 1H), 8.30 (s, 1H), 8.00-8.20 (m, 3H), 7.60-7.70 (m, 1H), 7.25-7.50 (m, 5H), 7.00-7.20 (m, 1H), 6.65-6.80 (m, 1H), 5.55-5.70 (m, 1H), 3.00-3.10 (m, 1H), 2.80-2.95 (m, 1H), 2.55 (s, 1H), 2.00-2.20 (m, 1H).

### Intermediate 15-1: (S)-3-(3-(1-amino-2,3-dihydro-1H-inden-5-yl)-3H-imidazo[4,5-b]pyridin-2-yl)pyridin-2-amine

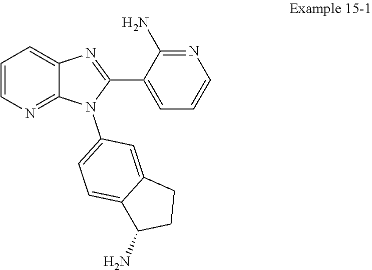 [SMILES: Nc1ncccc1-c1nc2cccnc2n1-c1ccc2c(c1)CC[C@@H]2N]

Intermediate 15-1 was prepared in a manner analogous to Intermediate 13-2 using 3-nitropyridin-2-amine instead of 6-methyl-3-nitropyridin-2-amine. MS (ESI) calculated for C₂₀H₁₈N₆: 342.16 m/z, found 343.10 [M+H]⁺.

### Example 16: N-[(1S)-5-[2-(2-aminopyridin-3-yl)-5-phenylimidazo[4,5-b]pyridin-3-yl]-2,3-dihydro-1H-inden-1-yl]-2-fluoro-5-formyl-4-hydroxybenzamide

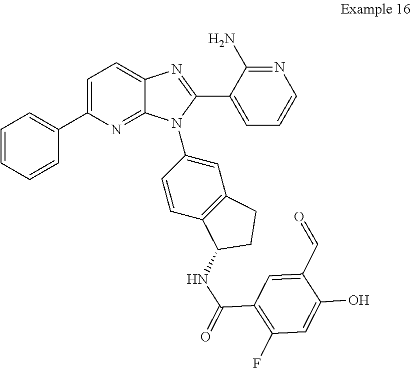 [SMILES: Nc1ncccc1-c1nc2ccc(-c3ccccc3)nc2n1-c1ccc2c(c1)CC[C@@H]2NC(=O)c1cc(C=O)c(O)cc1F]

### Synthetic Route:

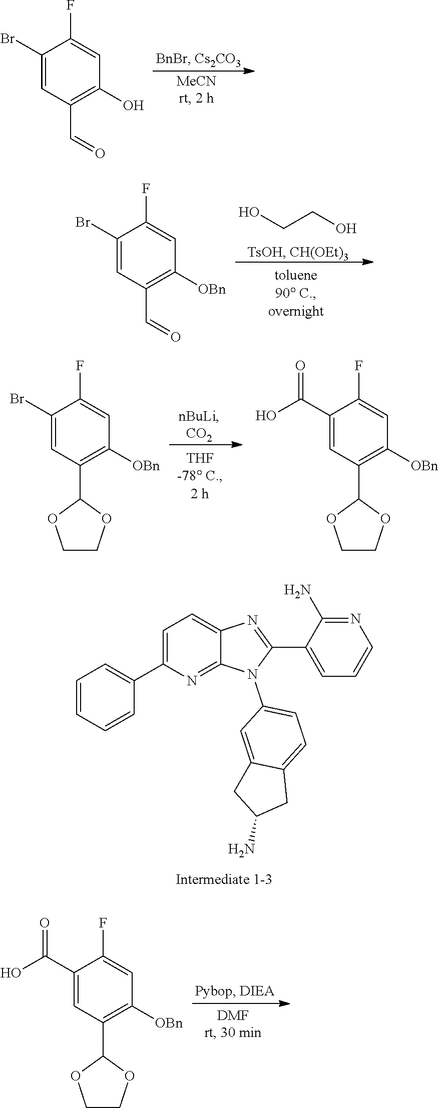

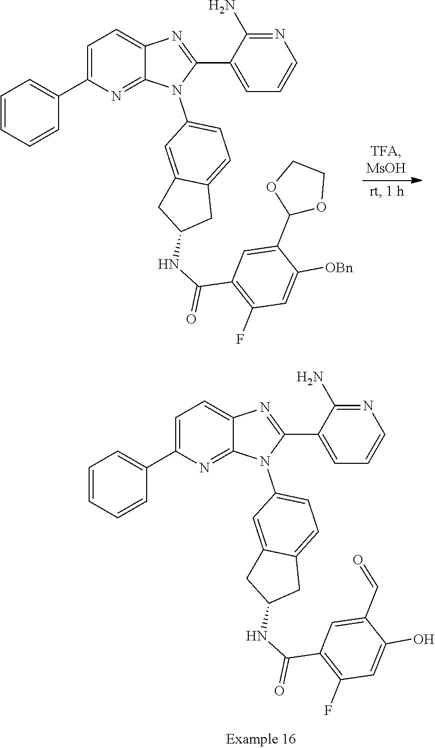 [SMILES: Nc1ncccc1-c1nc2ccc(-c3ccccc3)nc2n1-c1ccc2c(c1)C[C@@H](NC(=O)c1cc(C3OCCO3)c(OCc3ccccc3)cc1F)C2 ; Nc1ncccc1-c1nc2ccc(-c3ccccc3)nc2n1-c1ccc2c(c1)C[C@@H](NC(=O)c1cc(C=O)c(O)cc1F)C2]

### Step 1: Synthesis of 2-(benzyloxy)-5-bromo-4-fluorobenzaldehyde

To a solution of 5-bromo-4-fluoro-2-hydroxybenzaldehyde (5 g, 23 mmol, 1 equiv) in acetonitrile (45 mL) were added benzyl bromide (5 g, 34.2 mmol, 1.5 equiv) and cesium carbonate (14.88 g, 46 mmol, 2 equiv). The resulting mixture was stirred at room temperature for 2 h. The mixture was then extracted with ethyl acetate (30 mL×3). The combined organic layers were dried over anhydrous sodium sulfate, filtered, and concentrated in vacuo to give an off-white solid. Petroleum ether (50 mL) was added, and the mixture was stirred for 20 min. The resulting mixture was filtered and concentrated to afford 2-(benzyloxy)-5-bromo-4-fluorobenzaldehyde (5.5 g, 77% yield) as an off-white solid. MS (ESI) calculated for Cl₄H₁₀BrFO₂: 307.98 m/z, found 309.00 [M+H]⁺.

### Step 2: Synthesis of 2-[2-(benzyloxy)-5-bromo-4-fluorophenyl]-1,3-dioxolane

A mixture of 2-(benzyloxy)-5-bromo-4-fluorobenzaldehyde (5.46 g, 17.7 mmol, 1 equiv), ethylene glycol (8.77 g, 141 mmol, 8 equiv), p-toluenesulfonic acid (0.3 g, 1.77 mmol, 10 mol %), triethyl orthoformate (20.94 g, 141 mmol, 8 equiv) and toluene (50 mL) was stirred at 90° C. overnight. Water (40 mL) was added, and the resulting mixture was extracted with ethyl acetate (30 mL×3). The combined organic layers were concentrated in vacuo and the resulting residue was purified on a silica gel column using an eluent of 8% ethyl acetate in petroleum ether to afford the 2-[2-(benzyloxy)-5-bromo-4-fluorophenyl]-1,3-dioxolane as a white solid (2.34 g, 35% yield). MS (ESI) calculated for Cl₆H₁₄BrFO₃: 352.01 m/z, found 352.95 [M+H]⁺.

### Step 3: Synthesis of 4-(benzyloxy)-5-(1,3-dioxolan-2-yl)-2-fluorobenzoic acid

To a cooled (−78° C.) solution of 2-[2-(benzyloxy)-5-bromo-4-fluorophenyl]-1,3-dioxolane (2.34 g, 6.21 mmol, 1 equiv) in tetrahydrofuran (50 mL) was added dropwise n-butyllithium solution (2.5 M in hexane, 3.73 mL, 9.32 mmol, 1.5 equiv) under N₂ atmosphere. The reaction mixture was stirred at −78° C. for 30 mins. Then CO₂ was bubbled through the reaction mixture for 10 minutes and the mixture stirred for an additional 1 h at −78° C. under CO₂. The reaction was quenched with saturated aqueous ammonium chloride (20 mL) and the mixture was extracted with ether/ethyl acetate (2×30 mL). The combined organic extracts were washed with brine (10 mL), dried over anhydrous sodium sulfate, and concentrated in vacuo. The crude residue was purified by reverse-phase column chromatography on C18 silica gel using an eluent of 24% acetonitrile in water to afford 4-(benzyloxy)-5-(1,3-dioxolan-2-yl)-2-fluorobenzoic acid as a yellow solid (317 mg, 15% yield). MS (ESI) calculated for C₁₇H₁₅FO₅: 318.09 m/z, found 317.05 [M−H]⁻.

### Step 4: Synthesis of N-[(1S)-5-[2-(2-aminopyridin-3-yl)-5-phenylimidazo[4,5-b]pyridin-3-yl]-2,3-dihydro-1H-inden-1-yl]-4-(benzyloxy)-5-(1,3-dioxolan-2-yl)-2-fluorobenzamide

A mixture of 3-{3-[(1S)-1-amino-2,3-dihydro-1H-inden-5-yl]-5-phenylimidazo[4,5-b]pyridin-2-yl}pyridin-2-amine (Intermediate 1-3) (154 mg, 0.37 mmol, 1 equiv), N,N-dimethylformamide (5 mL), N,N-diisopropylethylamine (238 mg, 1.84 mmol. 5 equiv), Pybop (287 mg, 0.55 mmol, 1.5 equiv) and 4-(benzyloxy)-5-(1,3-dioxolan-2-yl)-2-fluorobenzoic acid (150 mg, 0.44 mmol, 1.2 equiv) was stirred at room temperature for 30 min. The resulting mixture was purified by reverse-phase column chromatography on C18 silica gel using an eluent of 65% acetonitrile in water to afford N-[(1S)-5-[2-(2-aminopyridin-3-yl)-5-phenylimidazo[4,5-b]pyridin-3-yl]-2,3-dihydro-1H-inden-1-yl]-4-(benzyloxy)-5-(1,3-dioxolan-2-yl)-2-fluorobenzamide as an off-white solid (200 mg, 67% yield). MS (ESI) calculated for C₄₃H₃₅FN₆O₄: 718.27 m/z, found 719.25 [M+H]⁺.

### Step 5: Synthesis of N-[(1S)-5-[2-(2-aminopyridin-3-yl)-5-phenylimidazo[4,5-b]pyridin-3-yl]-2,3-dihydro-1H-inden-1-yl]-2-fluoro-5-formyl-4-hydroxybenzamide (Example 16)

N-[(1S)-5-[2-(2-aminopyridin-3-yl)-5-phenylimidazo[4,5-b]pyridin-3-yl]-2,3-dihydro-1H-inden-1-yl]-4-(benzyloxy)-5-(1,3-dioxolan-2-yl)-2-fluorobenzamide (200 mg, 0.25 mmol, 1 equiv) was dissolved in TFA (0.5 mL) and methanesulfonic acid (2.5 mL). The resulting solution was stirred at room temperature for 1 h. The solution was then neutralized with saturated aqueous sodium bicarbonate. The resulting mixture was extracted with ethyl acetate (3×30 mL). The combined organic layers were dried over anhydrous sodium sulfate, filtered and concentrated in vacuo. The resulting residue was purified by Preparative HPLC on a Xselect CSH Prep Fluoro-Phenyl Column (19×250, 5 μm) using a 28-58% gradient of acetonitrile in water (+0.05% TFA) to afford N-[(1S)-5-[2-(2-aminopyridin-3-yl)-5-phenylimidazo[4,5-b]pyridin-3-yl]-2,3-dihydro-1H-inden-1-yl]-2-fluoro-5-formyl-4-hydroxybenzamide (Example 16) (24.6 mg, 17%) as an off-white solid. MS (ESI) calculated for C₃₄H₂₅FN₆O₃: 584.20 m/z, found 585.20 [M+H]⁺. ¹H NMR (300 MHz, DMSO-d₆) δ (ppm): 10.22 (s, 1H), 8.32-8.35 (m, 1H), 8.01-8.08 (m, 5H), 7.74-7.77 (m, 1H), 7.38-7.57 (m, 6H), 6.78-6.88 (m, 2H), 5.54-5.59 (m, 1H), 2.90-3.02 (m, 2H), 2.51-2.52 (m, 1H), 2.36 (s, 1H), 2.02-2.09 (m, 1H), 1.23 (s, 1H). ¹⁹F NMR (300 MHz, DMSO-d₆) δ (ppm): −101.80.

### Example 17: N-[(1S)-5-[2-(2-aminopyridin-3-yl)-5-phenylimidazo[4,5-b]pyridin-3-yl]-2,3-dihydro-1H-inden-1-yl]-5-formyl-4-hydroxy-2-methoxybenzamide

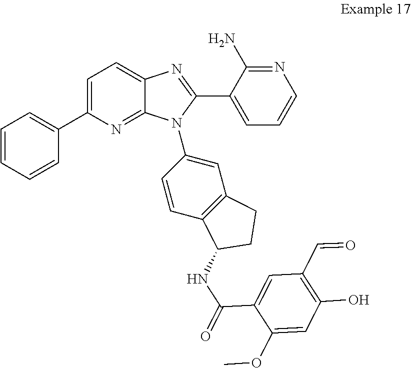 [SMILES: COc1cc(O)c(C=O)cc1C(=O)N[C@H]1CCc2cc(-n3c(-c4cccnc4N)nc4ccc(-c5ccccc5)nc43)ccc21]

Example 17 was prepared in a manner analogous to Example 16 using 5-bromo-2-hydroxy-4-methoxybenzaldehyde in place of 5-bromo-4-fluoro-2-hydroxybenzaldehyde. MS (ESI) calculated for C₃₅H₂₈N₆O₄: 596.22 m/z, found 597.25 [M+H]⁺. ¹H NMR (300 MHz, DMSO-d₆) δ (ppm): 10.10 (s, 1H), 8.32-8.35 (m, 1H), 8.14 (s, 1H), 8.02-8.09 (m, 4H), 7.73-7.76 (m, 1H), 7.37-7.51 (m, 6H), 6.78-6.82 (m, 1H), 6.65 (s, 1H), 5.56-5.62 (m, 1H), 3.90 (s, 3H), 2.90-3.00 (m, 2H), 2.51-2.52 (m, 1H), 2.04-2.11 (m, 1H).

### Example 18: N-[(1S)-5-[2-(2-aminopyridin-3-yl)-5-(pyridazin-3-yl)imidazo[4,5-b]pyridin-3-yl]-2,3-dihydro-1H-inden-1-yl]-3-formyl-4-hydroxybenzamide

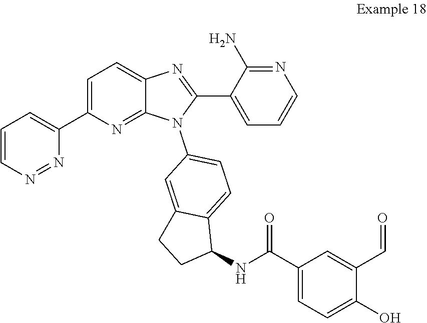 [SMILES: Nc1ncccc1-c1nc2ccc(-c3cccnn3)nc2n1-c1ccc2c(c1)CC[C@@H]2NC(=O)c1ccc(O)c(C=O)c1]

Example 18 was prepared in a manner analogous to Example 12 using Intermediate 18-3 in place of Intermediate 1-3 and Intermediate 5-1 in place of Intermediate 12-1. MS (ESI) calculated for C₃₂H₂₄N₈O₃: 568.20 m/z, found 569.20 [M+H]⁺. ¹H NMR (400 MHz, DMSO-d₆) δ (ppm): 10.29 (s, 1H), 9.85-9.86 (m, 1H), 9.33-9.34 (m, 1H), 8.45-8.47 (m, 1H), 8.30-8.32 (m, 3H), 8.08-8.26 (m, 2H), 7.80-7.82 (m, 1H), 7.48-7.80 (m, 1H), 7.38-7.48 (m, 2H), 7.07-7.09 (m, 1H), 6.81-6.84 (m, 1H), 5.61-5.65 (m, 1H), 3.02-3.06 (m, 1H), 2.89-2.93 (m, 1H), 2.50-2.51 (m, 1H), 2.11-2.14 (m, 1H).

### Intermediate 18-3: (S)-3-(3-(1-amino-2,3-dihydro-1H-inden-5-yl)-5-(pyridazin-3-yl)-3H-imidazo[4,5-b]pyridin-2-yl)pyridin-2-amine

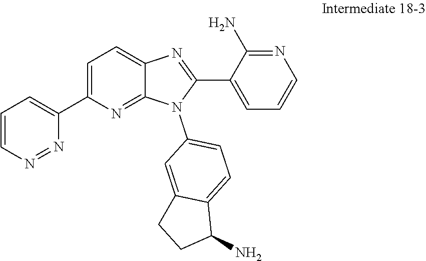 [SMILES: Nc1ncccc1-c1nc2ccc(-c3cccnn3)nc2n1-c1ccc2c(c1)CC[C@@H]2N]

### Synthetic Route:

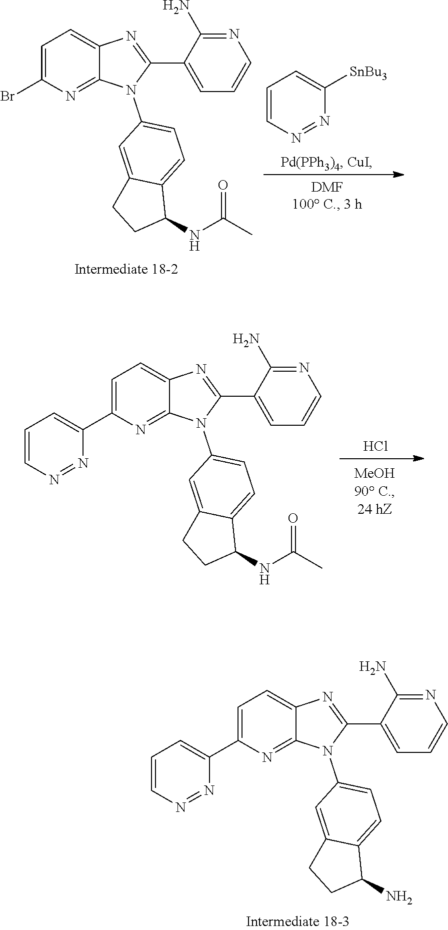

### Step 1: Synthesis of N-[(1S)-5-[2-(2-aminopyridin-3-yl)-5-(pyridazin-3-yl)imidazo[4,5-b]pyridin-3-yl]-2,3-dihydro-1H-inden-1-yl]acetamide

To a solution of N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (500 mg, 1.08 mmol, 1 equiv) in N,N-dimethylformamide (5 mL) was added 3-(tributylstannyl)pyridazine (797 mg, 2.16 mmol, 2 equiv), CuI (21 mg, 0.11 mmol, 0.1 equiv) and tetrakis(triphenylphosphine)palladium(0) (127 mg, 0.11 mmol, 0.1 equiv) The resulting mixture was stirred at 100° C. for 3 h under nitrogen. The resulting mixture was purified by reverse-phase flash chromatography on C18 silica gel using a 20-95% gradient of acetonitrile in water (+0.05% ammonium bicarbonate) with a 10 min hold at 70% acetonitrile to afford N-[(1S)-5-[2-(2-aminopyridin-3-yl)-5-(pyridazin-3-yl)imidazo[4,5-b]pyridin-3-yl]-2,3-dihydro-1H-inden-1-yl]acetamide (230 mg, 39%) as a red solid. MS (ESI) calculated for C₂₆H₂₂N₈O: 462.19 m/z, found 463.20 [M+H]⁺.

### Step 2: Synthesis of 3-{3-[(1S)-1-amino-2,3-dihydro-1H-inden-5-yl]-5-(pyridazin-3-yl)imidazo[4,5-b]pyridin-2-yl}pyridin-2-amine (Intermediate 18-3)

To a solution of N-[(1S)-5-[2-(2-aminopyridin-3-yl)-5-(pyridazin-3-yl)imidazo[4,5-b]pyridin-3-yl]-2,3-dihydro-1H-inden-1-yl]acetamide (230 mg, 0.497 mmol, 1 equiv) in MeOH (20 mL) was added HCl (20 mL, concentrated). The resulting mixture was stirred at 90° C. for 24 h. The solvent was removed by distillation under vacuum to afford 3-{3-[(1S)-1-amino-2,3-dihydro-1H-inden-5-yl]-5-(pyridazin-3-yl)imidazo[4,5-b]pyridin-2-yl}pyridin-2-amine (Intermediate 18-3) (200 mg, 88%) as a dark blue solid which was used in subsequent transformations without further purification. MS (ESI) calculated for C₂₄H₂₀N₈: 420.18 m/z, found 421.20 [M+H]⁺.

### Intermediate 18-2: (S)—N-(5-(2-(2-aminopyridin-3-yl)-5-bromo-3H-imidazo[4,5-b]pyridin-3-yl)-2,3-dihydro-1H-inden-1-yl)acetamide

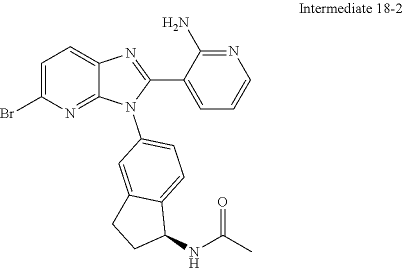 [SMILES: CC(=O)N[C@H]1CCc2cc(-n3c(-c4cccnc4N)nc4ccc(Br)nc43)ccc21]

### Synthetic Route:

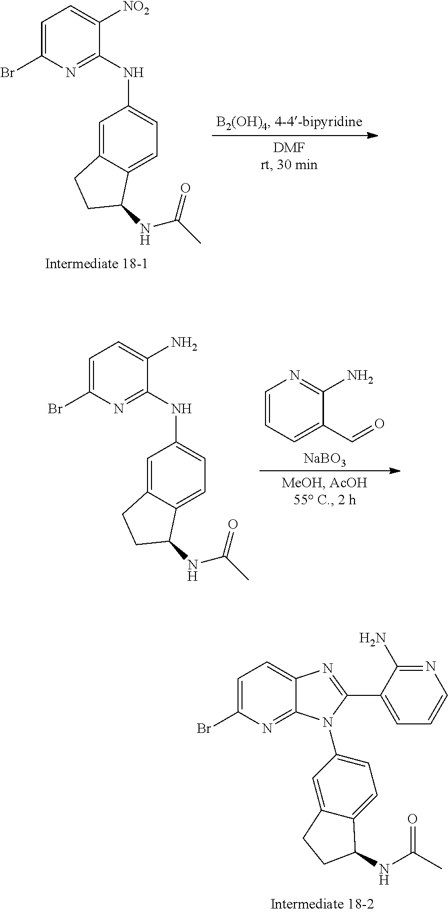

### Step 1: Synthesis of (S)—N-(5-((3-amino-6-bromopyridin-2-yl)amino)-2,3-dihydro-1H-inden-1-yl)acetamide

To a cooled (0° C.) solution of N-[(1S)-5-[(6-bromo-3-nitropyridin-2-yl)amino]-2,3-dihydro-1H-inden-1-yl]acetamide (Intermediate 18-1) (25 g, 64 mmol, 1 equiv) in N,N-dimethylformamide (250 mL) was added 4,4′-bipyridine (0.5 g, 2 mmol, 3 mol %), followed by hypodiboric acid (17.3 g, 0.192 mol, 3 equiv). The resulting mixture was stirred at 0° C. for 0.5 h. The reaction mixture was quenched by addition of 500 mL saturated aqueous ammonium chloride. The aqueous layer was extracted with ethyl acetate (3×250 mL). The combined organic layers were dried over anhydrous sodium sulfate, filtered and concentrated in vacuo. The resulting residue was purified by silica gel column chromatography, eluting with petroleum ether/dichloromethane/methanol (70:27:3) to afford N-[(1S)-5-[(3-amino-6-bromopyridin-2-yl)amino]-2,3-dihydro-1H-inden-1-yl]acetamide (16 g, 69%). MS (ESI) calculated for C₁₆H₁₇BrN₄O: 360.06, found 361.00 [M+H]⁺, 363.00 [M+2+H]⁺.

### Step 2: Synthesis of (S)—N-(5-(2-(2-aminopyridin-3-yl)-5-bromo-3H-imidazo[4,5-b]pyridin-3-yl)-2,3-dihydro-1H-inden-1-yl)acetamide (Intermediate 18-2)

A solution of N-[(1S)-5-[(3-amino-6-bromopyridin-2-yl)amino]-2,3-dihydro-1H-inden-1-yl]acetamide (15.5 g, 42.9 mmol, 1 equiv) in methanol (72 mL) and acetic acid (14 mL) was treated with 2-aminopyridine-3-carbaldehyde (6.29 g, 51.5 mmol, 1.2 equiv) followed by the addition of sodium perborate tetrahydrate (26.41 g, 172 mmol, 4 equiv) portion wise. The resulting mixture was stirred at 55° C. for 2 h. The reaction mixture was concentrated under reduced pressure and then brought to pH 8-9 with saturated aqueous sodium bicarbonate. The resulting precipitate was filtered. The filter cake was then washed with ethyl acetate (3×100 mL). The resulting filtrate was concentrated under reduced pressure and the residue was purified by silica gel column chromatography using a 0 to 20% gradient of ethyl acetate in petroleum ether followed by a 0 to 10% gradient of dichloromethane in methanol to provide (S)—N-(5-(2-(2-aminopyridin-3-yl)-5-bromo-3H-imidazo[4,5-b]pyridin-3-yl)-2,3-dihydro-1H-inden-1-yl)acetamide (Intermediate 18-2) (4 g, 18% yield). MS (ESI) calculated for C₂₂H₁₉BrN₆O: 462.08, found 463.00 [M+H]⁺, 465.00 [M+H+2]⁺.

### Intermediate 18-1: (S)—N-(5-((6-bromo-3-nitropyridin-2-yl)amino)-2,3-dihydro-1H-inden-1-yl)acetamide

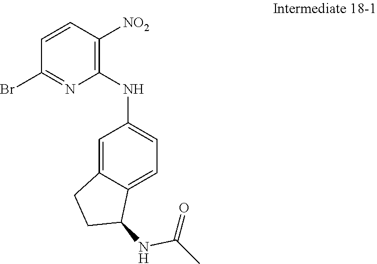 [SMILES: CC(=O)N[C@H]1CCc2cc(Nc3nc(Br)ccc3[N+](=O)[O-])ccc21]

### Synthetic Route

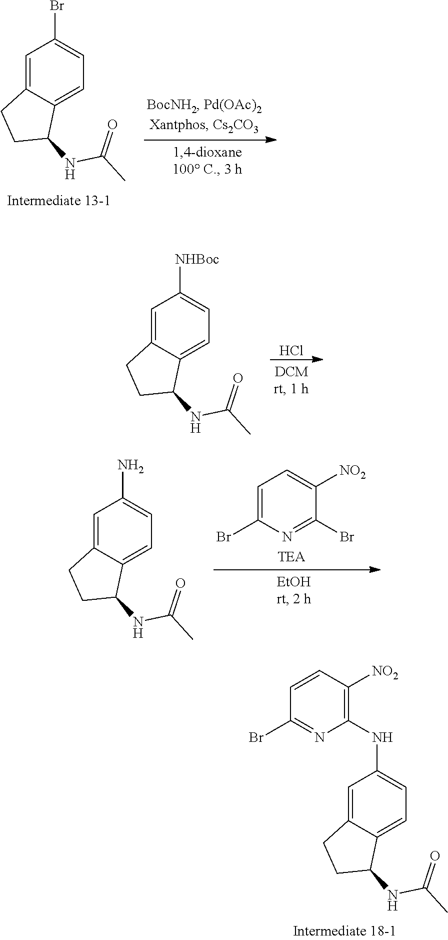 [SMILES: CC(=O)N[C@H]1CCc2cc(Br)ccc21 ; CC(=O)N[C@H]1CCc2cc(NC(=O)OC(C)(C)C)ccc21 ; Cl ; CC(=O)N[C@H]1CCc2cc(N)ccc21 ; O=[N+]([O-])c1ccc(Br)nc1Br ; CC(=O)N[C@H]1CCc2cc(Nc3nc(Br)ccc3[N+](=O)[O-])ccc21]

### Step 1: Synthesis of tert-butyl (S)-(1-acetamido-2,3-dihydro-1H-inden-5-yl)carbamate

To a mixture of N-[(1S)-5-bromo-2,3-dihydro-1H-inden-1-yl]acetamide (Intermediate 13-1) (40 g, 157 mmol, 1 equiv), tert-butyl carbamate (27.66 g, 236 mmol, 1.5 equiv), XantPhos (CAS: 161265-03-8) (9.11 g, 15.7 mmol, 10 mol %), Pd(OAc)₂ (3.54 g, 15.7 mmol, 10 mol %), and cesium carbonate (154 g, 472 mmol, 10 mol %) was added 1,4-dioxane (300 mL) under nitrogen atmosphere. The resulting mixture was stirred for 3 h at 100° C. The reaction mixture was quenched by addition of water (50 mL) and extracted with ethyl acetate (3×50 mL). The combined organic layers were dried over anhydrous sodium sulfate and concentrated under reduced pressure. The residue was purified by silica gel column chromatography using an eluent of petroleum ether/dichloromethane/methanol (70:27:3) to afford tert-butyl N-[(1S)-1-acetamido-2,3-dihydro-1H-inden-5-yl]carbamate (43.1 g, 48% yield). MS (ESI) calculated for C₁₆H₂₂N₂O₃: 290.16, found 289.05 [M−H]⁻.

### Step 2: Synthesis of (S)—N-(5-amino-2,3-dihydro-1H-inden-1-yl)acetamide

To a stirred solution of tert-butyl N-[(1S)-1-acetamido-2,3-dihydro-1H-inden-5-yl]carbamate (43.1 g, 148 mmol, 1 equiv) in dichloromethane (180 mL) was added 4N HCl in 1,4-dioxane (185 mL, 742 mmol, 5 equiv). The reaction mixture was stirred for 1 h at room temperature. The reaction mixture was concentrated in vacuo and re-crystallized from ethyl acetate to afford N-[(1S)-5-amino-2,3-dihydro-1H-inden-1-yl]acetamide (hydrochloride salt) (23 g, 81% yield) as a white solid. MS (ESI) calculated for C₁₁H₁₄N₂O: 190.11, found 191.15 [M+H]⁺.

### Step 3: (S)—N-(5-((6-bromo-3-nitropyridin-2-yl)amino)-2,3-dihydro-1H-inden-1-yl)acetamide (Intermediate 18-1)

N-[(1S)-5-amino-2,3-dihydro-1H-inden-1-yl]acetamide (17 g, 89 mmol), 2,6-dibromo-3-nitropyridine (25.19 g, 89.36 mmol) was dissolved in triethylamine (45.21 g, 446.8 mmol, 5 equiv) and ethanol (200 mL). The reaction mixture was stirred at room temperature for 2 h. The reaction mixture was quenched with 400 mL water and the precipitate was rinsed with 1:1 ethanol/water (800 mL:800 mL) to afford N-[(1S)-5-[(6-bromo-3-nitropyridin-2-yl)amino]-2,3-dihydro-1H-inden-1-yl]acetamide (Intermediate 18-1) (26 g, 74% yield) as an orange solid. MS (ESI) calculated for Cl₆H₁₅BrN₄O₃: 390.03, found 413.00 [M+Na]⁺, 415.00 [M+Na+2]⁺.

### Example 19: (S)—N-(5-(2-(2-aminopyridin-3-yl)-5-(3-(4-methylpiperazin-1-yl)phenyl)-3H-imidazo[4,5-b]pyridin-3-yl)-2,3-dihydro-1H-inden-1-yl)-3-formyl-4-hydroxybenzamide

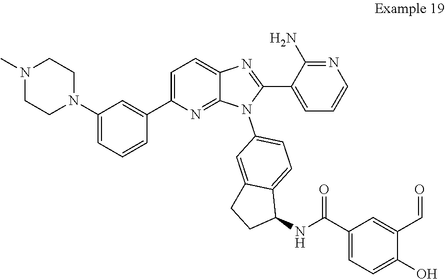 [SMILES: CN1CCN(c2cccc(-c3ccc4nc(-c5cccnc5N)n(-c5ccc6c(c5)CC[C@@H]6NC(=O)c5ccc(O)c(C=O)c5)c4n3)c2)CC1]

### Synthetic Route:

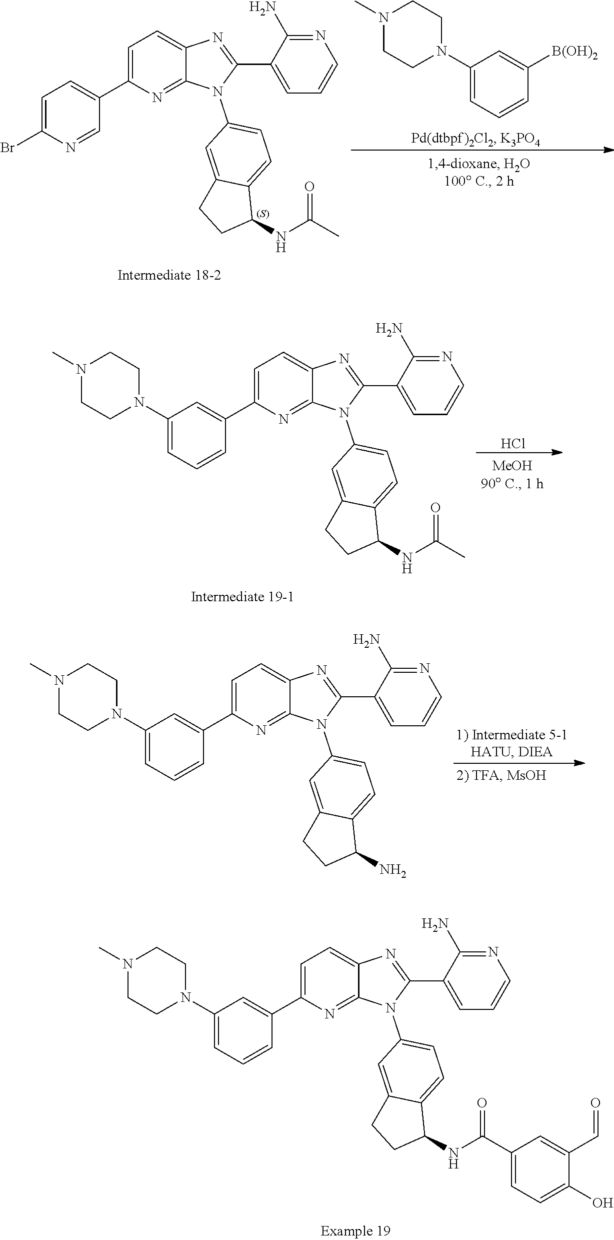

### Step 1: N-[(1S)-5-[2-(2-aminopyridin-3-yl)-5-[3-(4-methylpiperazin-1-yl)phenyl]imidazo[4,5-b]pyridin-3-yl]-2,3-dihydro-1H-inden-1-yl]acetamide (Intermediate 19-1)

A suspension of N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (20 mg, 0.043 mmol, 1 equiv), 3-(4-methylpiperazin-1-yl)phenylboronic acid (19 mg, 0.086 mmol, 2 equiv), Pd(dtbpf)Cl₂ (2.8 mg, 0.004 mmol, 0.1 equiv) and K₃PO₄ (27 mg, 0.13 mmol, 3 equiv) in 1,4-dioxane (0.8 mL) and H₂O (0.2 mL) was stirred at 100° C. for 2 h under N₂. The mixture was cooled to room temperature followed by addition of ethyl acetate (20 mL) and water (20 mL). The layers were separated, and the aqueous layer was extracted with ethyl acetate (10 mL×2). The combined organic layers were washed with brine (30 mL×3), dried with anhydrous sodium sulfate, filtered and concentrated. The residue was purified by silica gel column chromatography using a gradient of 0-30% ethyl acetate in petroleum ether to provide N-[(1S)-5-[2-(2-aminopyridin-3-yl)-5-[3-(4-methylpiperazin-1-yl)phenyl]imidazo[4,5-b]pyridin-3-yl]-2,3-dihydro-1H-inden-1-yl]acetamide (Intermediate 19-1) (280 mg, 58%) as yellow solid. MS (ESI) calculated for C₃₃H₃₄N₈O: 558.29 m/z, found 559.15 [M+H]⁺.

### Step 2: 3-{3-[(1S)-1-amino-2,3-dihydro-1H-inden-5-yl]-5-[3-(4-methylpiperazin-1-yl)phenyl]imidazo[4,5-b]pyridin-2-yl}pyridin-2-amine

To a suspension of N-[(1S)-5-[2-(2-aminopyridin-3-yl)-5-[3-(4-methylpiperazin-1-yl)phenyl]imidazo[4,5-b]pyridin-3-yl]-2,3-dihydro-1H-inden-1-yl]acetamide (Intermediate 19-1) (280 mg, 0.5 mmol, 1 equiv) in methanol (15 mL) was added HCl (5 mL, concentrated) at 0° C. The resulting mixture was stirred at 90° C. overnight. After concentration, the residue was dissolved in dichloromethane (20 mL) and adjusted to pH 9 with sodium hydroxide (2M, aqueous). Water (20 mL) was added, and the mixture was extracted with ethyl acetate (10 mL×3). The combined organic layers were washed with brine (30 mL×3), dried with anhydrous sodium sulfate, filtered and concentrated to provide 3-{3-[(1S)-1-amino-2,3-dihydro-1H-inden-5-yl]-5-[3-(4-methylpiperazin-1-yl)phenyl]imidazo[4,5-b]pyridin-2-yl}pyridin-2-amine (258 mg, 89%) as a yellow solid, which was used directly in the next step without further purification MS (ESI) calculated for C₃₁H₃₂N₈: 516.27 m/z, found 517.20 [M+H]⁺.

The remainder of the synthetic route was carried out in a manner analogous to Example 7 using the product from Step 2 vide supra in place of Intermediate 1-2 to give (S)—N-(5-(2-(2-aminopyridin-3-yl)-5-(3-(4-methylpiperazin-1-yl)phenyl)-3H-imidazo[4,5-b]pyridin-3-yl)-2,3-dihydro-1H-inden-1-yl)-3-formyl-4-hydroxybenzamide (Example 19). MS (ESI) calculated for C₃₉H₃₆N₈O₃: 664.29 m/z, found 665.20 [M+H]⁺. ¹H NMR (400 MHz, DMSO-d₆) δ 10.28 (s, 1H), 8.24-8.33 (m, 2H), 7.99-8.11 (m, 3H), 7.72-7.77 (m, 1H), 7.63 (s, 1H), 7.46-7.55 (m, 2H), 7.27-7.40 (m, 3H), 7.04-7.12 (m, 2H), 6.73-6.82 (m, 1H), 5.55-5.64 (m, 1H), 3.86-3.95 (m, 2H), 3.52 (s, 2H), 3.19 (s, 2H), 2.97-3.08 (m, 3H), 2.89-2.95 (m, 1H), 2.87 (s, 4H), 2.02-2.15 (m, 1H).

### Example 20: (S)—N-(5-(2-(2-aminopyridin-3-yl)-5-(4-fluorophenyl)-3H-imidazo[4,5-b]pyridin-3-yl)-2,3-dihydro-1H-inden-1-yl)-3-formyl-4-hydroxybenzamide

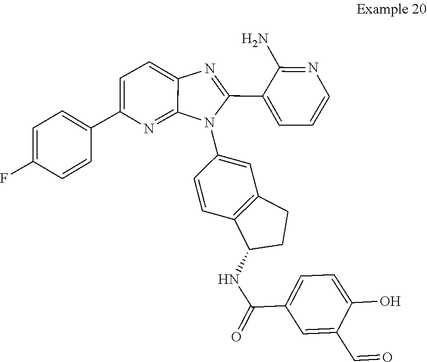 [SMILES: Nc1ncccc1-c1nc2ccc(-c3ccc(F)cc3)nc2n1-c1ccc2c(c1)CC[C@@H]2NC(=O)c1ccc(O)c(C=O)c1]

Example 20 was prepared in a manner analogous to Example 19 using 4-fluorophenylboronic acid in place of 3-(4-methylpiperazin-1-yl)phenylboronic acid. MS (ESI) calculated for C₃₄H₂₅FN₆O₃: 584.20 m/z, found 585.20 [M+H]⁺. ¹HNMR (400 MHz, DMSO-d₆) δ (ppm): 10.23-10.31 (m, 1H), 8.89-9.01 (m, 1H), 8.19-8.32 (m, 2H), 7.98-8.13 (m, 4H), 7.88-7.95 (m, 1H), 7.35-7.39 (m, 2H), 7.21-7.31 (m, 4H), 6.98-7.09 (m, 1H), 6.37-6.45 (m, 1H), 5.52-5.64 (m, 1H), 2.82-3.09 (m, 2H), 2.01-2.16 (m, 1H). ¹⁹FNMR (400 MHz, DMSO-d₆) δ (ppm): −113.37.

### Example 21: N-[(1S)-5-[2-(2-aminopyridin-3-yl)-5-(2-fluorophenyl)imidazo[4,5-b]pyridin-3-yl]-2,3-dihydro-1H-inden-1-yl]-3-formyl-4-hydroxybenzamide

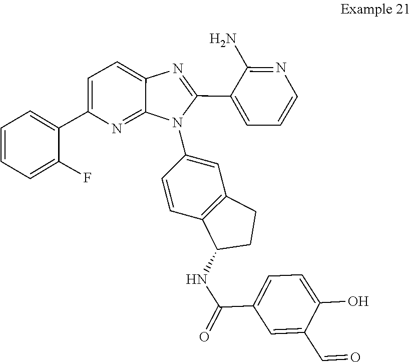 [SMILES: Nc1ncccc1-c1nc2ccc(-c3ccccc3F)nc2n1-c1ccc2c(c1)CC[C@@H]2NC(=O)c1ccc(O)c(C=O)c1]

Example 21 was prepared in a manner analogous to Example 19 using 2-fluorophenylboronic acid in place of 3-(4-methylpiperazin-1-yl)phenylboronic acid. MS (ESI) calculated for C₃₄H₂₅FN₆O₃: 584.20 m/z, found 585.20 [M+H]⁺. ¹H-NMR (400 MHz, DMSO-d₆) δ (ppm): 10.3 (s, 1H), 8.3-8.4 (m, 2H), 8.1 (m, 2H), 7.7-7.9 (m, 3H), 7.5 (m, 2H), 7.3-7.4 (m, 4H), 7.1 (m, 1H), 6.7-6.8 (m, 1H), 5.6 (m, 1H), 3.0-3.2 (m, 1H), 2.8-3.0 (m, 1H), 2.3-2.4 (m, 1H), 2.1 (m, 1H). ¹⁹F-NMR (400 MHz, DMSO-d₆) δ (ppm): −117.36.

### Example 22: N-[(1S)-5-[2-(2-aminopyridin-3-yl)-5-phenylimidazo[4,5-b]pyridin-3-yl]-2,3-dihydro-1H-inden-1-yl]-2,3-difluoro-5-formyl-4-hydroxybenzamide

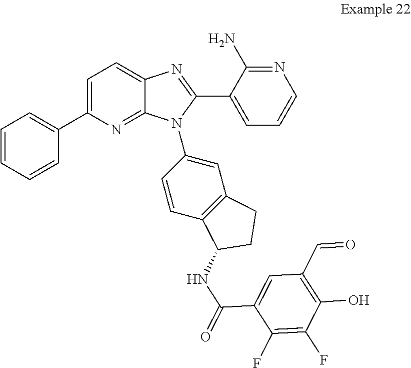 [SMILES: Nc1ncccc1-c1nc2ccc(-c3ccccc3)nc2n1-c1ccc2c(c1)CC[C@@H]2NC(=O)c1cc(C=O)c(O)c(F)c1F]

### Synthetic Route:

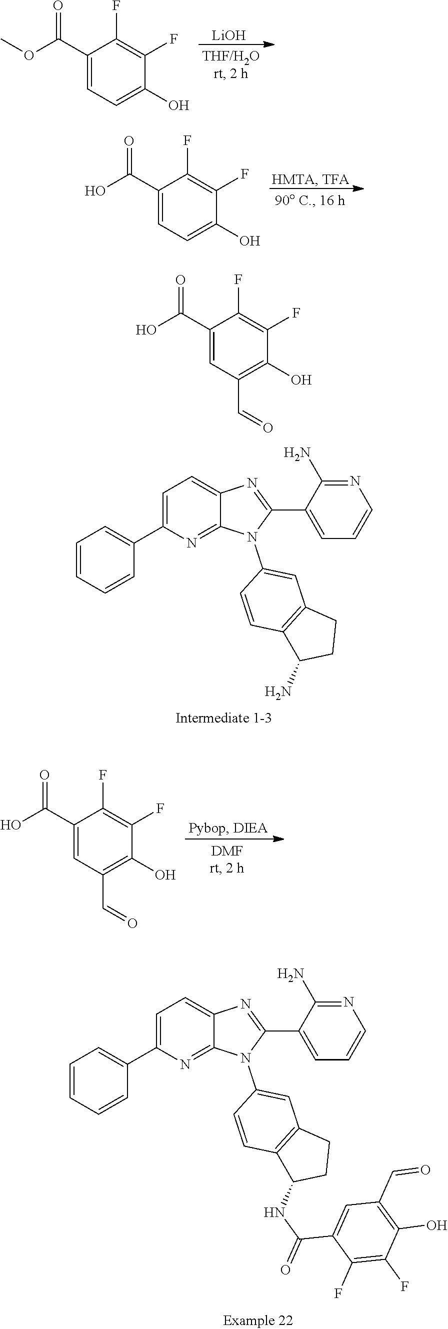

### Step 1: Synthesis of 2,3-difluoro-4-hydroxybenzoic acid

To a solution of methyl 2,3-difluoro-4-hydroxybenzoate (1.5 g, 7.97 mmol, 1 equiv) in tetrahydrofuran (20 mL) was added a solution of lithium hydroxide (1.53 g, 63.8 mmol, 8 equiv) in water (10 mL). The resulting mixture was stirred at room temperature for 2 h. The solvent was removed by distillation under vacuum to provide crude 2,3-difluoro-4-hydroxybenzoic acid (880 mg, 63%) as a white solid, which was used in the next step without further purification. MS (ESI) calculated for C₇H₄F₂O₃: 174.01 m/z, found 172.95 [M−H]⁻.

### Step 2: Synthesis of 2,3-difluoro-5-formyl-4-hydroxybenzoic acid

To a solution of 2,3-difluoro-4-hydroxybenzoic acid (500 mg, 2.87 mmol, 1 equiv) in TFA (10 mL) was added 1,3,5,7-tetraazaadamantane (604 mg, 4.3 mmol, 1.5 equiv). The resulting suspension was stirred for 16 h at 90° C. The reaction was quenched by the addition of water at 0° C. The resulting mixture was extracted with ethyl acetate (50 mL×3). The combined organic layers were washed with water (50 mL×3) and dried over anhydrous sodium sulfate, filtered and concentrated in vacuo. The residue was washed with petroleum ether/ethyl acetate (100 mL/10 mL) giving 2,3-difluoro-5-formyl-4-hydroxybenzoic acid (389 mg, 66%) as a white solid. MS (ESI) calculated for C₈H₄F₂O₄: 202.01 m/z, found 203.65 [M+H]⁺.

### Step 3: Synthesis of N-[(1S)-5-[2-(2-aminopyridin-3-yl)-5-phenylimidazo[4,5-b]pyridin-3-yl]-2,3-dihydro-1H-inden-1-yl]-2,3-difluoro-5-formyl-4-hydroxybenzamide (Example 22)

To a stirred mixture of 3-{3-[(1S)-1-amino-2,3-dihydro-1H-inden-5-yl]-5-phenylimidazo[4,5-b]pyridin-2-yl}pyridin-2-amine (Intermediate 1-3) (50 mg, 0.12 mmol, 1 equiv) in N,N-dimethylformamide (3 mL) was added N,N-diisopropylethylamine (46 mg, 0.36 mmol, 3 equiv) and Pybop (93 mg, 0.18 mmol, 1.5 equiv). The resulting mixture was stirred at room temperature for 2 h. The mixture was neutralized to pH 7 with aqueous sodium bicarbonate. The resulting mixture was extracted with ethyl acetate (3×30 mL). The combined organic layers were dried over anhydrous sodium sulfate, filtered and concentrated in vacuo. The resulting residue was purified by Preparative HPLC on a XBridge Shield RP 18 OBD Column (30×150 mm, 5 μm) using a 30-60% gradient of acetonitrile in water (+0.05% TFA) to afford N-[(1S)-5-[2-(2-aminopyridin-3-yl)-5-phenylimidazo[4,5-b]pyridin-3-yl]-2,3-dihydro-1H-inden-1-yl]-2,3-difluoro-5-formyl-4-hydroxybenzamide (Example 22) (11.2 mg, 15%) as a yellow solid. MS (ESI) calculated for C₃₄H₂₄F₂N₆O₃: 602.19 m/z, found 603.25 [M+H]⁺. ¹H-NMR (400 MHz, DMSO-d₆) δ (ppm): 10.19 (s, 1H), 8.25-8.32 (m, 1H), 7.91-8.05 (m, 4H), 7.65-7.85 (m, 2H), 7.32-7.50 (m, 6H), 6.71-6.79 (m, 1H), 5.50-5.59 (m, 1H), 2.85-3.05 (m, 1H), 2.83-2.95 (m, 1H), 2.41-2.59 (m, 1H), 1.96-2.09 (m, 1H). ¹⁹F-NMR (400 MHz, DMSO-d₆) δ (ppm): −128.30, −158.12.

### Example 23: N-[(1S)-5-[2-(2-aminopyridin-3-yl)-5-(pyridin-3-yl)imidazo[4,5-b]pyridin-3-yl]-2,3-dihydro-1H-inden-1-yl]-3-formyl-4-hydroxybenzamide

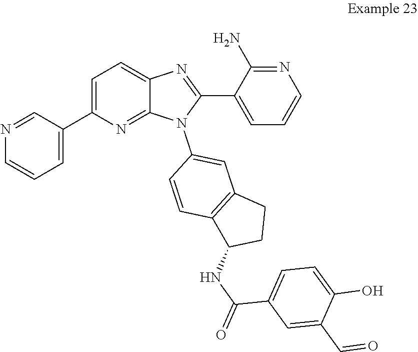 [SMILES: Nc1ncccc1-c1nc2ccc(-c3cccnc3)nc2n1-c1ccc2c(c1)CC[C@@H]2NC(=O)c1ccc(O)c(C=O)c1]

Example 23 was prepared in a manner analogous to Example 19 using pyridin-3-ylboronic acid in place of 3-(4-methylpiperazin-1-yl)phenylboronic acid. MS (ESI) calculated for C₃₃H₂₅N₇O₃: 567.20 m/z, found 568.15 [M+H]⁺. ¹H NMR (400 MHz, DMSO-d₆) δ 10.29 (s, 1H), 9.32-9.39 (m, 1H), 8.66-8.79 (m, 2H), 8.41-8.49 (m, 1H), 8.26-8.31 (m, 1H), 8.12-8.19 (m, 1H), 8.04-8.11 (m, 2H), 7.73-7.82 (m, 2H), 7.48-7.51 (m, 1H), 7.33-7.39 (m, 2H), 7.09-7.11 (m, 1H), 6.79-6.95 (m, 1H), 5.53-5.62 (m, 1H), 2.99-3.12 (m, 1H), 2.85-2.91 (m, 1H), 2.39-2.42 (m, 1H), 2.08-2.17 (m, 1H).

### Example 24: N-[(1S)-5-[2-(2-aminopyridin-3-yl)-5-(pyridin-4-yl)imidazo[4,5-b]pyridin-3-yl]-2,3-dihydro-1H-inden-1-yl]-3-formyl-4-hydroxybenzamide

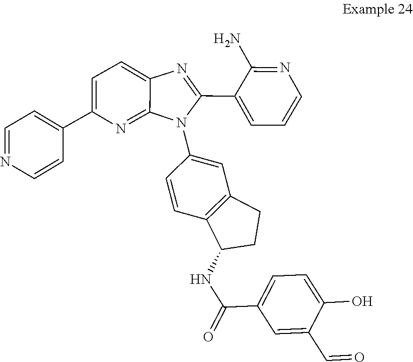 [SMILES: Nc1ncccc1-c1nc2ccc(-c3ccncc3)nc2n1-c1ccc2c(c1)CC[C@@H]2NC(=O)c1ccc(O)c(C=O)c1]

Example 24 was prepared in a manner analogous to Example 19 using pyridin-4-ylboronic acid in place of 3-(4-methylpiperazin-1-yl)phenylboronic acid. MS (ESI) calculated for C₃₃H₂₅N₇O₃: 567.20 m/z, found 568.20 [M+H]⁺. ¹H NMR (400 MHz, DMSO-d₆) δ (ppm): 10.26 (s, 1H), 8.61-8.92 (m, 2H), 8.08-8.32 (m, 3H), 7.92-8.05 (m, 4H), 7.26-7.38 (m, 4H), 6.89-7.01 (m, 1H), 6.41-6.53 (m, 1H), 5.53-5.66 (m, 1H), 2.81-3.09 (m, 2H), 2.02-2.16 (m, 1H), 1.08-1.28 (m, 1H).

### Example 25: (S)—N-(5-(2-(2-aminopyridin-3-yl)-5-(pyridin-2-yl)-3H-imidazo[4,5-b]pyridin-3-yl)-2,3-dihydro-1H-inden-1-yl)-3-formyl-4-hydroxybenzamide

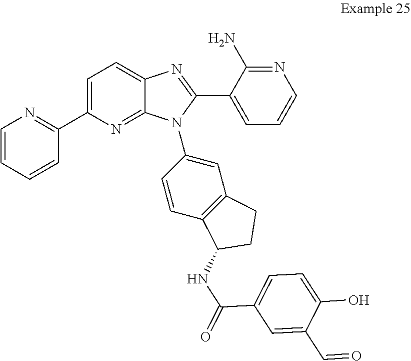 [SMILES: Nc1ncccc1-c1nc2ccc(-c3ccccn3)nc2n1-c1ccc2c(c1)CC[C@@H]2NC(=O)c1ccc(O)c(C=O)c1]

Example 25 was prepared in a manner analogous to Example 26 using pyridin-2-ylboronic acid in place of 3-fluorophenylboronic acid. MS (ESI) calculated for C₃₃H₂₅N₇O₃: 567.61 m/z, found 568.20 [M+H]⁺. ¹H NMR (400 MHz, DMSO-d₆) δ (ppm): 10.28 (s, 1H), 8.60-8.75 (m, 1H), 8.45-8.60 (m, 1H), 8.20-8.45 (m, 1H), 8.12-8.20 (m, 1H), 7.98-8.12 (m, 2H), 7.90-7.98 (m, 1H), 7.55-7.85 (m, 1H), 7.30-7.50 (m, 4H), 7.00-7.22 (m, 1H), 6.60-6.90 (m, 1H), 5.53-5.72 (m, 1H), 3.00-3.15 (m, 1H), 2.80-3.00 (m, 1H), 2.55-2.65 (m, 1H), 2.00-2.20 (m, 1H). Example 26: N-[(1S)-5-[2-(2-aminopyridin-3-yl)-5-(3-fluorophenyl)imidazo[4,5-b]pyridin-3-yl]-2,3-dihydro-1H-inden-1-yl]-3-formyl-4-hydroxybenzamide

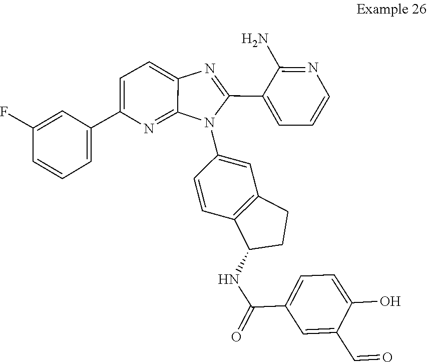 [SMILES: Nc1ncccc1-c1nc2ccc(-c3cccc(F)c3)nc2n1-c1ccc2c(c1)CC[C@@H]2NC(=O)c1ccc(O)c(C=O)c1]

### Synthetic Route:

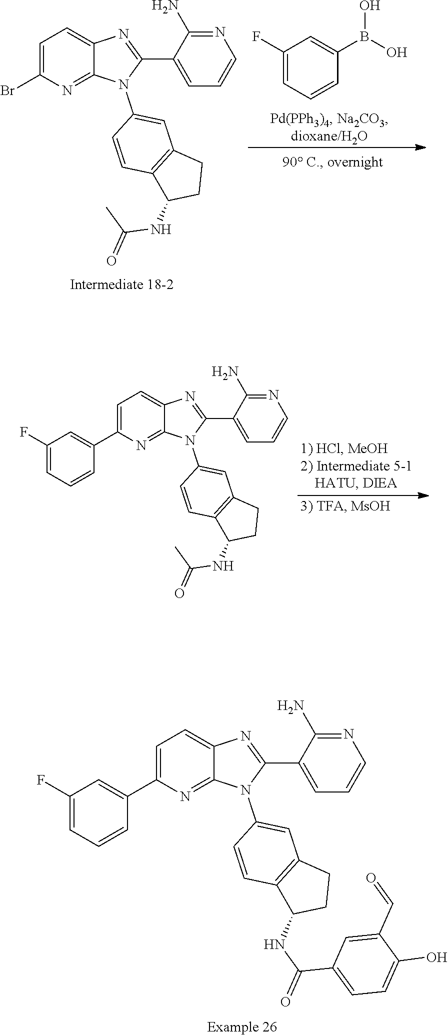

### Step 1: Synthesis of N-[(1S)-5-[2-(2-aminopyridin-3-yl)-5-(3-fluorophenyl)imidazo[4,5-b]pyridin-3-yl]-2,3-dihydro-1H-inden-1-yl]acetamide

A mixture of N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (500 mg, 1.08 mmol, 1 equiv) and 3-fluorophenylboronic acid (302 mg, 2.16 mmol, 2 equiv) was dissolved in 1,4-dioxane (10 mL) and water (2.5 mL). tetrakis(triphenylphosphine)palladium(0) (187 mg, 0.16 mmol, 0.15 equiv) and sodium carbonate (231 mg, 2.16 mmol, 2 equiv) were added and the mixture was stirred overnight at 90° C. The reaction was quenched with water (100 mL) and extracted with ethyl acetate (100 mL×3). The combined organic layers were washed with brine (100 mL), dried over anhydrous sodium sulfate, filtered and concentrated in vacuo. The resulting residue was purified by silica gel column chromatography using an eluent of 20% ethyl acetate in petroleum ether to afford N-[(1S)-5-[2-(2-aminopyridin-3-yl)-5-(3-fluorophenyl)imidazo[4,5-b]pyridin-3-yl]-2,3-dihydro-1H-inden-1-yl]acetamide (200 mg, 35%) as a yellow oil. MS (ESI) calculated for C₂₈H₂₃FN₆O: 478.53 m/z, found 479.20 [M+H]⁺.

The remainder of the synthesis was carried out in a manner analogous to Example 19 (starting from step 2) using the product from Step 1 vide supra in place of Intermediate 19-1 to provide N-[(1S)-5-[2-(2-aminopyridin-3-yl)-5-(3-fluorophenyl)imidazo[4,5-b]pyridin-3-yl]-2,3-dihydro-1H-inden-1-yl]-3-formyl-4-hydroxybenzamide (Example 26). MS (ESI) calculated for C₃₄H₂₅FN₆O₃: 584.61 m/z, found 585.15 [M+H]⁺. ¹H NMR (400 MHz, DMSO-d₆) δ (ppm): 10.26 (s, 1H), 8.90-9.12 (m, 1H), 8.13-8.36 (m, 2H), 8.01-8.13 (m, 1H), 7.92-8.01 (m, 1H), 7.68-7.90 (m, 2H), 7.42-7.60 (m, 1H), 7.15-7.42 (m, 5H), 6.90-7.15 (m, 1H), 6.25-6.65 (m, 1H), 5.45-5.80 (m, 1H), 2.98-3.10 (m, 1H), 2.75-2.98 (m, 1H), 2.40-2.50 (m, 1H), 1.90-2.10 (m, 1H).

### Example 27: 5-[2-(2-aminopyridin-3-yl)-5-phenylimidazo[4,5-b]pyridin-3-yl]-N-(3-formyl-4-hydroxyphenyl)-2,3-dihydro-1H-indene-1-carboxamide

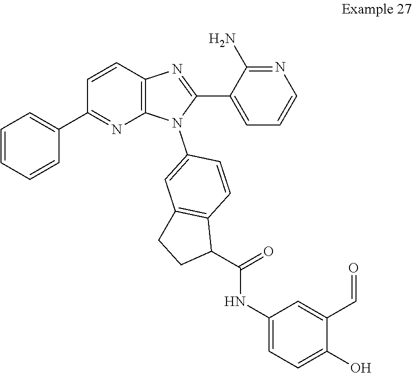 [SMILES: Nc1ncccc1-c1nc2ccc(-c3ccccc3)nc2n1-c1ccc2c(c1)CCC2C(=O)Nc1ccc(O)c(C=O)c1]

### Synthetic Route:

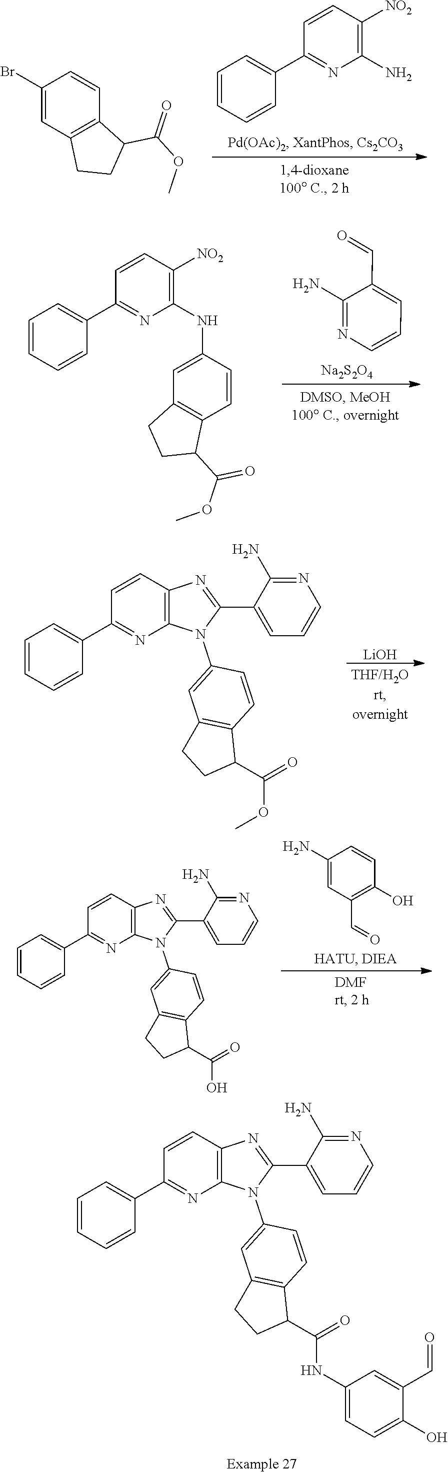

### Step 1: Synthesis of methyl 5-({4-nitro-[1,1′-biphenyl]-3-yl}amino)-2,3-dihydro-1H-indene-1-carboxylate

Methyl 5-bromo-2,3-dihydro-1H-indene-1-carboxylate (1 g, 3.9 mmol, 1 equiv) and 4-nitro-[1,1′-biphenyl]-3-amine (1.26 mg, 5.88 mmol, 1.5 equiv) were dissolved in 1,4-dioxane (20 mL). Pd(OAc)₂ (132 mg, 0.58 mmol, 0.15 equiv), XantPhos (CAS: 161265-03-8) (340 mg, 0.588 mmol, 0.15 equiv) and cesium carbonate (2.56 g, 7.84 mmol, 2 equiv) were added and the mixture was stirred for 2 h at 100° C. under N₂ atmosphere. The reaction was quenched with water (100 mL) and extracted with ethyl acetate (100 mL×3). The combined organic layers were washed with brine (100 mL), dried over anhydrous sodium sulfate, filtered and concentrated in vacuo. The resulting residue was purified by silica gel column chromatography using an eluent of 20% ethyl acetate in petroleum ether to afford methyl 5-({4-nitro-[1,1′-biphenyl]-3-yl}amino)-2,3-dihydro-1H-indene-1-carboxylate (320 mg, 19%) as a yellow oil. MS (ESI) calculated for C₂₃H₂₀N₂O₄: 388.42 m/z, found 389.15 [M+H]⁺.

### Step 2: Synthesis of methyl 5-[2-(2-aminopyridin-3-yl)-5-phenylimidazo[4,5-b]pyridin-3-yl]-2,3-dihydro-1H-indene-1-carboxylate

Methyl 5-({4-nitro-[1,1′-biphenyl]-3-yl}amino)-2,3-dihydro-1H-indene-1-carboxylate (280 mg, 0.72 mmol, 1 equiv), 2-aminopyridine-3-carbaldehyde (106 mg, 0.87 mmol, 1.2 equiv) and sodium dithionite (314 mg, 1.8 mmol, 2.5 equiv) were dissolved in DMSO (18 mL) and methanol (3.6 mL). The resulting mixture was stirred overnight at 100° C. The reaction mixture was concentrated and purified by reverse-phase flash chromatography on C18 silica gel using a 50 to 70% gradient of acetonitrile in water to afford methyl 5-[2-(2-aminopyridin-3-yl)-5-phenylimidazo[4,5-b]pyridin-3-yl]-2,3-dihydro-1H-indene-1-carboxylate (250 mg, 68%) as a yellow solid. MS (ESI) calculated for C₂₈H₂₃N₅O₂: 461.52 m/z, found 462.15 [M+H]⁺.

### Step 3: Synthesis of 5-[2-(2-aminopyridin-3-yl)-5-phenylimidazo[4,5-b]pyridin-3-yl]-2,3-dihydro-1H-indene-1-carboxylic acid

A solution of methyl 5-[2-(2-aminopyridin-3-yl)-5-phenylimidazo[4,5-b]pyridin-3-yl]-2,3-dihydro-1H-indene-1-carboxylate (240 mg, 0.52 mmol, 1 equiv) in tetrahydrofuran (10 mL) was treated with a solution of lithium hydroxide (37 mg, 1.6 mmol, 3 equiv) in water (2.5 mL) and the resulting mixture was stirred overnight at room temperature. The mixture was acidified to pH 6 with HCl (1M, aqueous). The resulting mixture was extracted with ethyl acetate (50 ml×3). The combined organic layers were dried over anhydrous sodium sulfate, filtered and concentrated in vacuo to afford 5-[2-(2-aminopyridin-3-yl)-5-phenylimidazo[4,5-b]pyridin-3-yl]-2,3-dihydro-1H-indene-1-carboxylic acid (180 mg, 77%) as a yellow oil which was used in the next step without further purification. MS (ESI) calculated for C₂₇H₂₁N₅O₂, 447.50 m/z, found 448.15 [M+H]⁺.

### Step 4: Synthesis of 5-[2-(2-aminopyridin-3-yl)-5-phenylimidazo[4,5-b]pyridin-3-yl]-N-(3-formyl-4-hydroxyphenyl)-2,3-dihydro-1H-indene-1-carboxamide (Example 27)

To a solution of 5-[2-(2-aminopyridin-3-yl)-5-phenylimidazo[4,5-b]pyridin-3-yl]-2,3-dihydro-1H-indene-1-carboxylic acid (160 mg, 0.36 mmol, 1 equiv), 5-aminosalicylaldehyde (74 mg, 0.54 mmol, 1.5 equiv) and N,N-diisopropylethylamine (139 mg, 1.08 mmol, 3 equiv) in N,N-dimethylformamide (10 mL) was added HATU (103 mg, 0.43 mmol, 1.2 equiv). The resulting mixture was stirred at room temperature for 2 h and then purified by reverse-phase flash column chromatography on C18 silica gel using a gradient of 20-95% acetonitrile in water (+0.05% ammonium bicarbonate) with a 40 min hold at 70% acetonitrile to afford impure product which was further purified by Preparative HPLC on a XBridge Prep OBD C18 column (30×150 mm, 5 μm) using a 36-66% gradient of acetonitrile in water (+10 mmol/L ammonium bicarbonate) to afford 5-[2-(2-aminopyridin-3-yl)-5-phenylimidazo[4,5-b]pyridin-3-yl]-N-(3-formyl-4-hydroxyphenyl)-2,3-dihydro-1H-indene-1-carboxamide (Example 27) (11.1 mg, 5.4%) as an off-white solid. MS (ESI) calculated for C₃₄H₂₆N₆O₃: 566.62, found 567.20 [M+H]⁺. ¹H NMR (400 MHz, DMSO-d₆) δ (ppm): 10.40-10.70 (m, 1H), 10.33 (s, 1H), 10.27 (s, 1H), 8.20-8.35 (m, 1H), 7.86-8.15 (m, 5H), 7.70-7.80 (m, 1H), 7.35-7.50 (m, 5H), 7.28-7.35 (m, 1H), 7.15-7.28 (m, 1H), 6.88-7.10 (m, 3H), 6.30-6.50 (m, 1H), 4.00-4.30 (m, 1H), 3.00-3.18 (m, 1H), 2.85-3.00 (m, 1H), 2.32-2.46 (m, 2H).

### Example 28: (S)—N-(5-(2-(2-aminopyridin-3-yl)-5-(1-methyl-1H-pyrazol-4-yl)-3H-imidazo[4,5-b]pyridin-3-yl)-2,3-dihydro-1H-inden-1-yl)-3-formyl-4-hydroxybenzamide

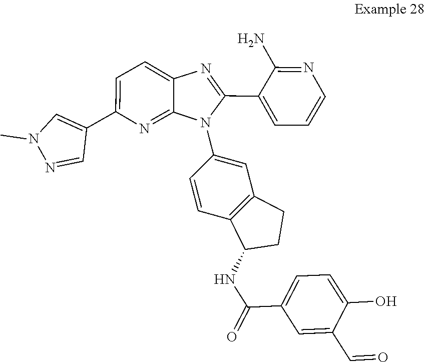 [SMILES: Cn1cc(-c2ccc3nc(-c4cccnc4N)n(-c4ccc5c(c4)CC[C@@H]5NC(=O)c4ccc(O)c(C=O)c4)c3n2)cn1]

Example 28 was prepared in a manner analogous to Example 19 using 1-methylpyrazol-4-ylboronic acid in place of 3-(4-methylpiperazin-1-yl)phenylboronic acid MS (ESI) calculated for C₃₂H₂₆N₈O₃: 570.21 m/z, found 572.05 [M+H]⁺. ¹H NMR (400 MHz, DMSO-d₆) δ (ppm): 10.32 (m, 1H), 8.96-8.98 (m, 1H), 8.31-8.32 (m, 1H), 8.13-8.15 (m, 3H), 8.00-8.01 (m, 1H), 7.93-7.99 (m, 1H), 7.66-7.68 (m, 1H), 7.33-7.36 (m, 2H), 7.24-7.26 (m, 2H), 7.06-7.08 (m, 1H), 6.94-6.95 (m, 2H), 6.42-6.43 (m, 1H) 5.63-5.65 (m, 1H), 3.87 (s, 3H), 3.01-3.03 (m, 1H), 3.93-3.94 (m, 1H), 2.50-2.51 (m, 1H), 2.11-2.12 (m, 1H).

### Example 29: N-[(1S)-5-[2-(2-aminopyridin-3-yl)-5-(pyrazin-2-yl)imidazo[4,5-b]pyridin-3-yl]-2,3-dihydro-1H-inden-1-yl]-3-formyl-4-hydroxybenzamide

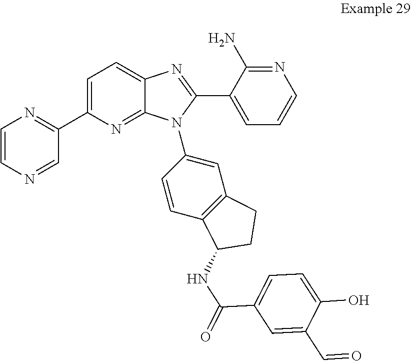 [SMILES: Nc1ncccc1-c1nc2ccc(-c3cnccn3)nc2n1-c1ccc2c(c1)CC[C@@H]2NC(=O)c1ccc(O)c(C=O)c1]

Example 29 was prepared in a manner analogous to Example 12 using Intermediate 29-1 in place of Intermediate 1-3 and Intermediate 5-1 in place of Intermediate 12-1. MS (ESI) calculated for C₃₂H₂₄N₈O₃: 568.20 m/z, found 569.25 [M+H]⁺. ¹H NMR (300 MHz, DMSO-d₆) δ (ppm): 10.31 (m, 1H), 9.30-9.36 (m, 1H), 8.97-8.99 (m, 1H), 8.72-8.73 (m, 1H), 8.67-8.68 (m, 1H), 8.39-8.42 (m, 2H), 8.31-8.34 (m, 1H), 8.08-8.10 (m, 1H), 8.03-8.04 (m, 1H), 7.44-7.47 (m, 1H), 7.29-7.32 (m, 3H), 7.00-7.06 (m, 3H), 6.44-6.51 (m, 1H), 5.65-5.68 (m, 1H), 3.03-3.06 (m, 1H), 2.91-2.94 (m, 1H), 2.50-2.56 (m, 1H), 2.08-2.11 (m, 1H).

### Intermediate 29-1 (S)-3-(3-(1-amino-2,3-dihydro-1H-inden-5-yl)-5-(pyrazin-2-yl)-3H-imidazo[4,5-b]pyridin-2-yl)pyridin-2-amine

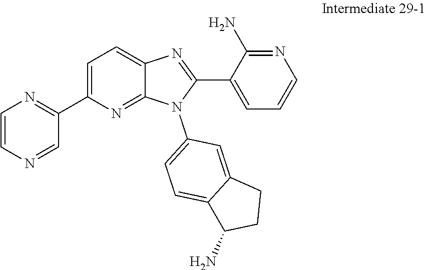 [SMILES: Nc1ncccc1-c1nc2ccc(-c3cnccn3)nc2n1-c1ccc2c(c1)CC[C@@H]2N]

Intermediate 29-1 was prepared in a manner analogous to Intermediate 18-3 using 2-(tributylstannyl)pyrazine in place of 3-(tributylstannyl)pyridazine. MS (ESI) calculated for C₂₄H₂₀N₈: 420.18 m/z, found 421.20 [M+H]⁺.

### Example 30: N-[(1S)-5-[2-(2-aminopyridin-3-yl)-5-phenylimidazo[4,5-b]pyridin-3-yl]-2,3-dihydro-1H-inden-1-yl]-4-formyl-3-hydroxybenzamide

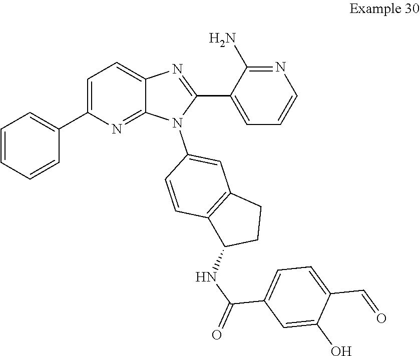 [SMILES: Nc1ncccc1-c1nc2ccc(-c3ccccc3)nc2n1-c1ccc2c(c1)CC[C@@H]2NC(=O)c1ccc(C=O)c(O)c1]

Example 30 was prepared in a manner analogous to Example 5 using Intermediate 30-1 in place of Intermediate 5-1. MS (ESI) calculated for C₃₄H₂₆N₆O₃: 566.20 m/z, found 567.25 [M+H]⁺. ¹H NMR (300 MHz, DMSO-d₆) δ (ppm): 10.31 (s, 1H), 8.25-8.28 (m, 1H), 7.96-8.03 (m, 4H), 7.71-7.74 (m, 1H), 7.31-7.50 (m, 8H), 7.25-7.28 (m, 1H), 6.43-6.47 (m, 1H), 5.60-5.65 (m, 1H), 2.85-3.05 (m, 2H), 2.51-2.52 (m, 1H), 2.06-2.33 (m, 1H).

### Intermediate 30-1: 4-(1,3-dioxolan-2-yl)-3-((4-methoxybenzyl)oxy)benzoic acid

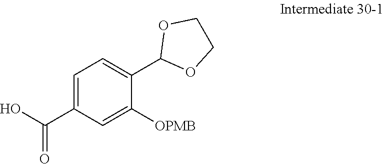

Intermediate 30-1 was prepared in a manner analogous to Intermediate 5-1 using Intermediate 6-1 in place of Intermediate 2-1. MS (ESI) calculated for C₁₈H₁₈O₆: 330.11 m/z, found 329.05 [M−H]⁻.

### Example 31: N-[(1S)-5-[2-(2-aminopyridin-3-yl)-5-phenylimidazo[4,5-b]pyridin-3-yl]-2,3-dihydro-1H-inden-1-yl]-2-fluoro-4-formyl-3-hydroxybenzamide

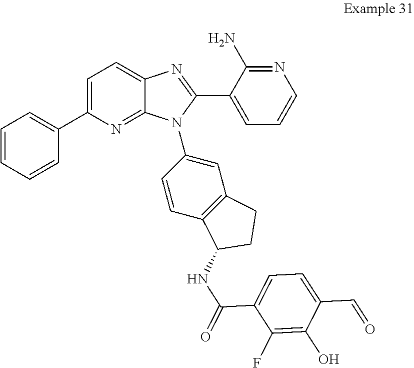 [SMILES: Nc1ncccc1-c1nc2ccc(-c3ccccc3)nc2n1-c1ccc2c(c1)CC[C@@H]2NC(=O)c1ccc(C=O)c(O)c1F]

Example 31 was prepared in a manner analogous to Example 12 using Intermediate 31-1 in place of Intermediate 12-1. MS (ESI) calculated for C₃₄H₂₅FN₆O₃: 584.20 m/z, found 585.20 [M+H]⁺. ¹H NMR (400 MHz, DMSO-d₆) δ (ppm): 10.29 (s, 1H), 9.11-9.13 (m, 1H), 8.26-8.28 (m, 1H), 7.97-8.01 (m, 4H), 7.40-7.49 (m, 7H), 7.26-7.28 (m, 1H), 7.11-7.15 (m, 1H), 6.44-6.47 (m, 1H), 5.58-5.60 (m, 1H), 2.90-3.01 (m, 2H), 2.51-2.53 (m, 1H), 1.06-2.06 (m, 1H). ¹⁹F-NMR (400 MHz, DMSO-d₆) δ (ppm): −137.03. Intermediate 31-1: 3-(benzyloxy)-4-(1,3-dioxolan-2-yl)-2-fluorobenzoic acid

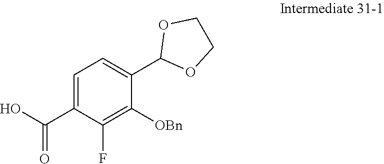 [SMILES: O=C(O)c1ccc(C2OCCO2)c(OCc2ccccc2)c1F]

Intermediate 31-1 was prepared in a manner analogous to Intermediate 36-1 using 4-bromo-3-fluoro-2-hydroxybenzaldehyde in place of 5-bromo-2-hydroxy-3-methoxybenzaldehyde. Crude Intermediate 31-1 was purified by reverse-phase flash column chromatography on C18 silica gel using a 20-95% gradient of acetonitrile in water (+0.05% ammonium bicarbonate) with a 10 minute hold at 70% acetonitrile to afford 3-(benzyloxy)-4-(1,3-dioxolan-2-yl)-2-fluorobenzoic acid (Intermediate 31-1) as a white solid. MS (ESI) calculated for C₁₇H₁₅FO₅: 318.05 m/z, found 319.05 [M+H]⁺.

### Example 32: N-[(1S)-5-[2-(2-aminopyridin-3-yl)-5-cyclopropoxyimidazo[4,5-b]pyridin-3-yl]-2,3-dihydro-1H-inden-1-yl]-3-formyl-4-hydroxybenzamide

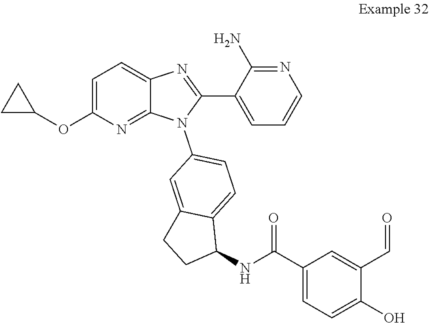 [SMILES: Nc1ncccc1-c1nc2ccc(OC3CC3)nc2n1-c1ccc2c(c1)CC[C@@H]2NC(=O)c1ccc(O)c(C=O)c1]

Example 32 was prepared in a manner analogous to Example 12 using Intermediate 32-2 in place of Intermediate 1-3 and Intermediate 5-1 in place of Intermediate 12-1. MS (ESI) calculated for C₃₁H₂₆N₆O₄: 546.20 m/z, found 547.10 [M+H]⁺. ¹H NMR (400 MHz, DMSO-d₆) δ 10.27 (s, 1H), 8.87-8.99 (m, 1H), 8.24-8.31 (m, 1H), 8.05-8.15 (m, 2H), 7.93-8.00 (m, 1H), 7.26-7.35 (m, 2H), 7.12-7.22 (m, 2H), 7.02-7.09 (m, 1H), 6.90-6.98 (m, 1H), 6.41-6.46 (m, 1H), 5.52-5.66 (m, 1H), 3.99-4.10 (m, 1H), 2.94-3.06 (m, 1H), 2.80-2.92 (m, 1H), 2.43-2.49 (m, 1H), 2.00-2.14 (m, 1H), 0.60-0.79 (m, 4H).

### Intermediate 32-2: (S)-3-(3-(1-amino-2,3-dihydro-1H-inden-5-yl)-5-cyclopropoxy-3H-imidazo[4,5-b]pyridin-2-yl)pyridin-2-amine

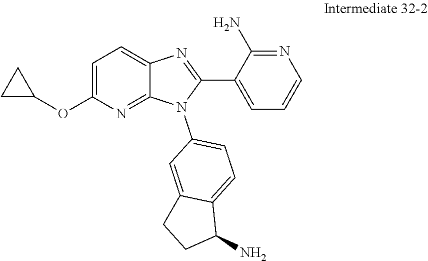 [SMILES: Nc1ncccc1-c1nc2ccc(OC3CC3)nc2n1-c1ccc2c(c1)CC[C@@H]2N]

Intermediate 32-2 was prepared in a manner analogous to Intermediate 13-2 using Intermediate 32-1 in place of 6-methyl-3-nitropyridin-2-amine. MS (ESI) calculated for C₂₃H₂₂N₆O: 398.19 m/z, found 399.05 [M+H]⁺.

### Intermediate 32-1: 6-cyclopropoxy-3-nitropyridin-2-amine

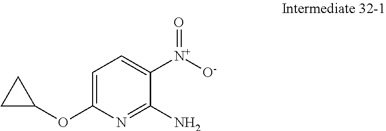 [SMILES: Nc1nc(OC2CC2)ccc1[N+](=O)[O-]]

### Synthetic Route:

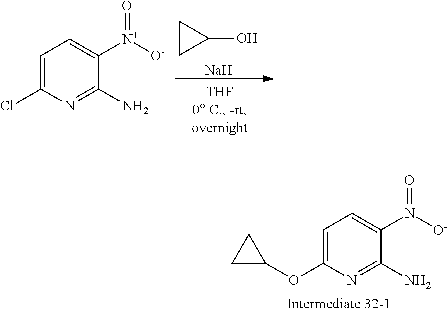

### Step 1: Synthesis of 6-cyclopropoxy-3-nitropyridin-2-amine (Intermediate 32-1)

Cyclopropanol (1.34 g, 23 mmol, 2 equiv) was dissolved in tetrahydrofuran (10 mL) and cooled to 0° C. under nitrogen atmosphere. Sodium hydride (60% dispersion in mineral oil) (0.92 g, 23 mmol, 2 equiv) was added and the resulting mixture was stirred at room temperature for 20 minutes. 6-chloro-3-nitropyridin-2-amine (2 g, 11 mmol, 1 equiv) was then added and the resulting mixture was stirred at room temperature overnight. The mixture was concentrated in vacuo and purified by flash column chromatography on silica gel column using a 0-50% gradient of ethyl acetate in petroleum ether to afford 6-cyclopropoxy-3-nitropyridin-2-amine (Intermediate 32-1) (1.5 g, 49%) as a yellow solid. MS (ESI) calculated for C₈H₉N₃O₃: 195.06 m/z, found 196.00 [M+H]⁺. ¹H NMR (400 MHz, DMSO-d₆) δ 8.23-8.32 (m, 1H), 8.10-8.19 (m, 2H), 6.13-6.22 (m, 1H), 3.90 (s, 1H), 0.69-0.86 (m, 4H).

### Example 33: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-formyl-4-hydroxybenzamide

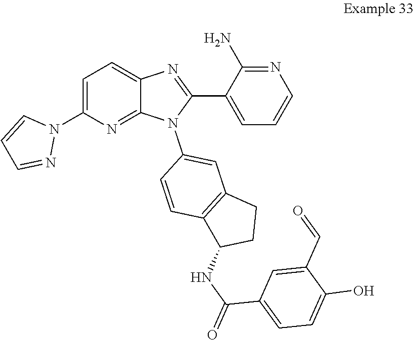 [SMILES: Nc1ncccc1-c1nc2ccc(-n3cccn3)nc2n1-c1ccc2c(c1)CC[C@@H]2NC(=O)c1ccc(O)c(C=O)c1]

### Synthetic Route:

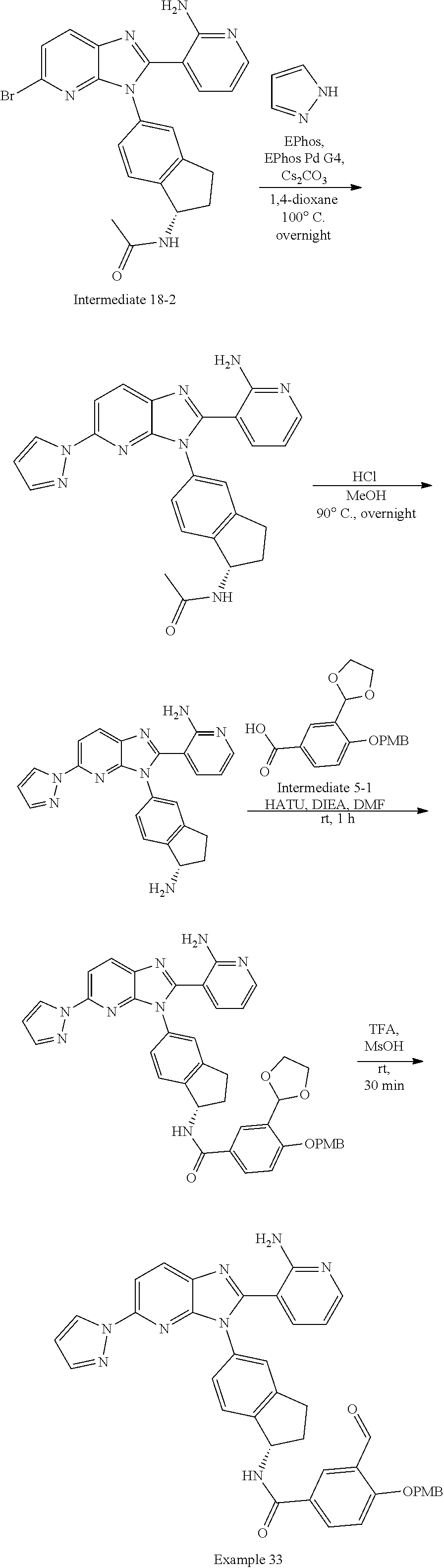

### Step 1: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]acetamide

A mixture of N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (200 mg, 0.43 mmol, 1 equiv), pyrazole (59 mg, 0.86 mmol, 2 equiv), EPhos (CAS: 2118959-55-8) (23 mg, 0.043 mmol, 0.1 equiv), EPhos Pd G4 (CAS: 2132978-44-8) (40 mg, 0.043 mmol, 0.1 equiv) and cesium carbonate (1.69 g, 5.20 mmol, 3 equiv) in 1,4-dioxane (5 mL) was stirred at 100° C. overnight. The mixture was cooled to room temperature and taken up into ethyl acetate (20 mL) and water (20 mL). The layers were separated, and the aqueous layer was extracted with ethyl acetate (10 mL×2). The combined organic layers were washed with brine (30 mL×3), dried with anhydrous sodium sulfate, filtered and concentrated. The residue was purified by silica gel column chromatography using a 0-30% gradient of ethyl acetate in petroleum ether to afford N-[(1S)-5-[2-(2-aminopyridin-3-yl)-5-(pyrazol-1-yl)imidazo[4,5-b]pyridin-3-yl]-2,3-dihydro-1H-inden-1-yl]acetamide (120 mg, 55%) as a light-yellow solid. MS (ESI) calculated for C₂₅H₂₂N₈O: 450.19 m/z, found 451.15 [M+H]⁺.

### Step 2: Synthesis of 3-{3-[(1S)-1-amino-2,3-dihydro-1H-inden-5-yl]-5-(pyrazol-1-yl)imidazo[4,5-b]pyridin-2-yl}pyridin-2-amine

To a suspension of N-[(1S)-5-[2-(2-aminopyridin-3-yl)-5-(pyrazol-1-yl)imidazo[4,5-b]pyridin-3-yl]-2,3-dihydro-1H-inden-1-yl]acetamide (120 mg, 0.27 mmol, 1 equiv) in methanol (10 mL) was added HCl (10 mL, concentrated) at 0° C. The mixture was then stirred at 90° C. overnight. The mixture was allowed to cool to room temperature and concentrated in vacuo. The residue was dissolved in dichloromethane (20 mL) and the pH was adjusted to 9 with NaOH (2M, aqueous). Water (20 mL) was added, and the mixture was extracted with ethyl acetate (10 mL×3). The combined organic layers were washed with brine (30 mL×3), dried with anhydrous sodium sulfate, filtered and concentrated in vacuo to provide 3-{3-[(1S)-1-amino-2,3-dihydro-1H-inden-5-yl]-5-(pyrazol-1-yl)imidazo[4,5-b]pyridin-2-yl}pyridin-2-amine (100 mg, 77%) as a yellow solid, which was used directly in the next step without further purification. MS (ESI) calculated for C₂₃H₂₀N₈: 408.18 m/z, found 409.15 [M+H]⁺.

### Step 3: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

A suspension of 3-{3-[(1S)-1-amino-2,3-dihydro-1H-inden-5-yl]-5-(pyrazol-1-yl)imidazo[4,5-b]pyridin-2-yl}pyridin-2-amine (80 mg, 0.20 mmol, 1 equiv), 3-(1,3-dioxolan-2-yl)-4-[(4-methoxyphenyl)methoxy]benzoic acid (Intermediate 5-1) (129 mg, 0.39 mmol, 2 equiv) and N,N-diisopropylethylamine (76 mg, 0.59 mmol, 3 equiv) in N,N-dimethylformamide (2 mL) was stirred at room temperature for 10 min. HATU (111 mg, 0.29 mmol, 1.5 equiv) was added and the resulting mixture was stirred at room temperature for 1 h. Water (20 mL) was added and the mixture was extracted with ethyl acetate (10 mL×2). The combined organic layers were washed with brine (30 mL×3), dried with anhydrous sodium sulfate, filtered and concentrated in vacuo. The residue was purified by flash column chromatography on silica gel using a gradient of 0-30% ethyl acetate in petroleum ether to provide N-[(1S)-5-[2-(2-aminopyridin-3-yl)-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (90 mg, 64%) as a yellow solid. MS (ESI) calculated for C₄₁H₃₆N₈O₅: 720.28 m/z, found 721.30 [M+H]⁺.

### Step 4: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-formyl-4-hydroxybenzamide (Example 33)

A solution of N-[(1S)-5-[2-(2-aminopyridin-3-yl)-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (80 mg, 0.11 mmol, 1 equiv) in TFA (5 mL) and methanesulfonic acid (1 mL) was stirred at room temperature for 0.5 h. The mixture was concentrated and brought to pH 8 with sodium bicarbonate. Water (20 mL) was added, and the mixture was extracted with ethyl acetate (20 mL×3). The combined organic layers were washed with brine (30 mL×3), dried with anhydrous sodium sulfate, filtered and concentrated in vacuo. The residue was purified by Preparative HPLC on a Xselect CSH C18 Column (30×150 mm, 5 μm) using a 21 to 51% gradient of acetonitrile in water (+0.1% formic acid) to provide (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-formyl-4-hydroxybenzamide (Example 33) (10.1 mg, 15.97%) as a white solid. MS (ESI) calculated for C₃₁H₂₄N₈O₃: 556.20 m/z, found 557.15 [M+H]⁺. ¹H NMR (400 MHz, DMSO-d₆) δ 10.17 (s, 1H), 8.88-9.02 (m, 1H), 8.15-8.40 (m, 3H), 7.83-8.09 (m, 3H), 7.75 (s, 1H), 7.12-7.47 (m, 4H), 6.96-7.10 (m, 1H), 6.43-6.61 (m, 2H), 5.40-5.60 (m, 1H), 2.92-3.04 (m, 1H), 2.78-2.90 (m, 1H), 2.47 (s, 1H), 1.83-2.12 (m, 1H).

### Example 34: N-[(1S)-5-[2-(2-aminopyridin-3-yl)-5-phenylimidazo[4,5-b]pyridin-3-yl]-2,3-dihydro-1H-inden-1-yl]-2-chloro-5-formyl-4-hydroxybenzamide

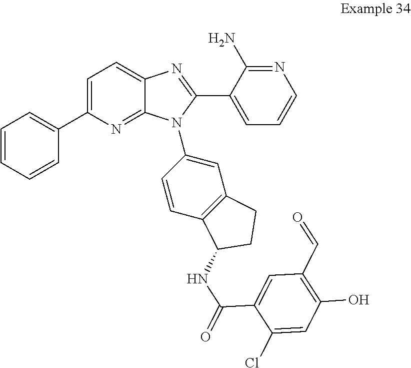 [SMILES: Nc1ncccc1-c1nc2ccc(-c3ccccc3)nc2n1-c1ccc2c(c1)CC[C@@H]2NC(=O)c1cc(C=O)c(O)cc1Cl]

### Synthetic Route:

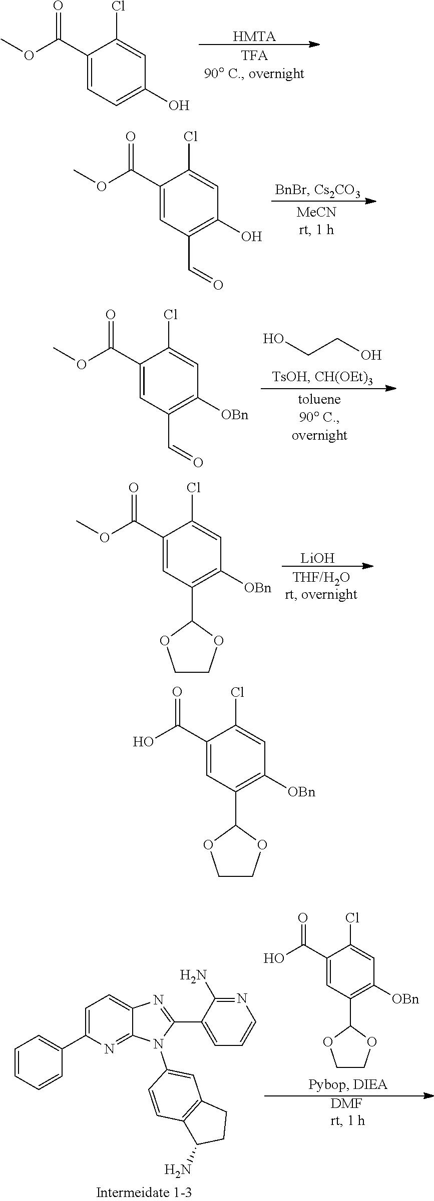

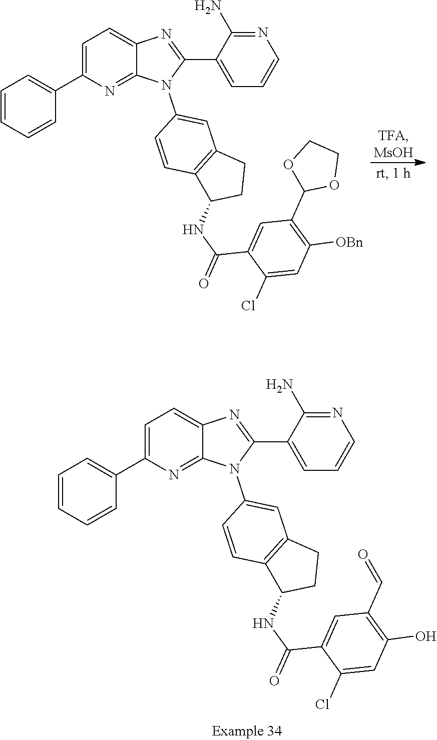 [SMILES: Nc1ncccc1-c1nc2ccc(-c3ccccc3)nc2n1-c1ccc2c(c1)CC[C@@H]2NC(=O)c1cc(C2OCCO2)c(OCc2ccccc2)cc1Cl ; Nc1ncccc1-c1nc2ccc(-c3ccccc3)nc2n1-c1ccc2c(c1)CC[C@@H]2NC(=O)c1cc(C=O)c(O)cc1Cl]

### Step 1: Synthesis of methyl 2-chloro-5-formyl-4-hydroxybenzoate

To a solution of methyl 2-chloro-4-hydroxybenzoate (1 g, 5.36 mmol, 1 equiv) in TFA (10 mL) was added 1,3,5,7-tetraazaadamantane (1.13 g, 8.04 mmol, 1.5 equiv). The resulting solution was stirred overnight at 90° C. The resulting mixture was concentrated under reduced pressure. The residue was purified by reverse-phase flash chromatography on C18 silica gel using a 10-50% gradient of acetonitrile in water to provide methyl 2-chloro-5-formyl-4-hydroxybenzoate (0.1 g, 8.7% yield) as a light yellow solid. MS (ESI) calculated for C₉H₇ClO₄: 214.00 m/z, found 212.95 [M−H]⁻.

### Step 2: Synthesis of methyl 4-(benzyloxy)-2-chloro-5-formylbenzoate

To a solution of methyl 2-chloro-5-formyl-4-hydroxybenzoate (100 mg, 0.47 mmol, 1 equiv) in acetonitrile (2 mL) was added benzyl bromide (120 mg, 0.70 mmol, 1.5 equiv) and cesium carbonate (304 mg, 0.93 mmol, 2 equiv). The resulting mixture was stirred at room temperature for 1 h. The reaction was quenched by addition of water at 0° C. and extracted with ethyl acetate (50 mL×3). The combined organic layers were washed with water (50 mL×3), dried over anhydrous sodium sulfate, filtered and concentrated in vacuo. The residue was washed with petroleum ether to afford methyl 4-(benzyloxy)-2-chloro-5-formylbenzoate (70 mg, 49% yield) as a white solid. MS (ESI) calculated For Cl₆H₁₃ClO₄: 304.05 m/z, found 305.15 [M+H]⁺.

### Step 3: Synthesis of methyl 4-(benzyloxy)-2-chloro-5-(1,3-dioxolan-2-yl)benzoate

To a solution of methyl 4-(benzyloxy)-2-chloro-5-formylbenzoate (1 g, 4.25 mmol, 1 equiv) in toluene (10 mL) was added ethylene glycol (1.85 g, 29.7 mmol, 7 equiv), triethyl orthoformate (1.89 g, 12.7 mmol, 3 equiv) and p-toluenesulfonic acid (0.04 g, 0.21 mmol, 0.05 equiv). The resulting solution was stirred at room temperature for 10 min and overnight at 90° C. The reaction mixture was cooled to 0° C. and quenched by the addition of saturated aqueous sodium bicarbonate. The resulting mixture was extracted with ethyl acetate (10 mL×3). The combined organic layers were washed with water (10 mL×3), dried over anhydrous sodium sulfate, filtered and concentrated in vacuo. The resulting residue was purified by silica gel column chromatography using a 0-17% gradient of ethyl acetate in petroleum ether to afford methyl 4-(benzyloxy)-2-chloro-5-(1,3-dioxolan-2-yl)benzoate (60 mg, 75% yield) as a colorless oil. MS (ESI) calculated for C₁₈H₁₇ClO₅: 348.08 m/z, found 349.10 [M+H]⁺.

### Step 4: Synthesis of 4-(benzyloxy)-2-chloro-5-(1,3-dioxolan-2-yl)benzoic acid

To a cooled (0° C.) solution of methyl 4-(benzyloxy)-2-chloro-5-(1,3-dioxolan-2-yl)benzoate (60 mg, 0.17 mmol, 1 equiv) in tetrahydrofuran (6 mL) was added a solution of lithium hydroxide (12 mg, 0.52 mmol, 3 equiv) in water. The resulting solution was stirred overnight at room temperature then partially concentrated in vacuo. The mixture was brought to pH 7 with HCl (2 N, aqueous). The resulting suspension was filtered to provide 4-(benzyloxy)-2-chloro-5-(1,3-dioxolan-2-yl)benzoic acid (32 mg, 56% yield) as a white solid, which was used directly in the next step without further purification MS (ESI) calculated for C₁₇H₁₅ClO₅: 334.06 m/z, found 335.00 [M+H]⁺.

### Step 5: Synthesis of N-[(1S)-5-[2-(2-aminopyridin-3-yl)-5-phenylimidazo[4,5-b]pyridin-3-yl]-2,3-dihydro-1H-inden-1-yl]-4-(benzyloxy)-2-chloro-5-(1,3-dioxolan-2-yl)benzamide

To a suspension of 3-{3-[(1S)-1-amino-2,3-dihydro-1H-inden-5-yl]-5-phenylimidazo[4,5-b]pyridin-2-yl}pyridin-2-amine (Intermediate 1-3) (31 mg, 0.074 mmol, 1 equiv) in N,N-dimethylformamide (1.5 mL) was added 4-(benzyloxy)-2-chloro-5-(1,3-dioxolan-2-yl)benzoic acid (25 mg, 0.074 mmol, 1 equiv), Pybop (58 mg, 0.11 mmol, 1.5 equiv) and N,N-diisopropylethylamine (29 mg, 0.22 mmol, 3 equiv). The reaction mixture was stirred at room temperature for 2 h. The mixture was purified by reverse-phase flash chromatography on C18 silica gel using a 10-70% gradient of acetonitrile in water (+0.05% ammonium bicarbonate) to provide N-[(1S)-5-[2-(2-aminopyridin-3-yl)-5-phenylimidazo[4,5-b]pyridin-3-yl]-2,3-dihydro-1H-inden-1-yl]-4-(benzyloxy)-2-chloro-5-(1,3-dioxolan-2-yl)benzamide (30 mg, 55% yield) as a light yellow solid. MS (ESI) calculated for C₄₃H₃₅ClN₆O₄: 734.24 m/z, found 735.30 [M+H]⁺.

### Step 6: Synthesis of N-[(1S)-5-[2-(2-aminopyridin-3-yl)-5-phenylimidazo[4,5-b]pyridin-3-yl]-2,3-dihydro-1H-inden-1-yl]-2-chloro-5-formyl-4-hydroxybenzamide (Example 34)

To a solution of N-[(1S)-5-[2-(2-aminopyridin-3-yl)-5-phenylimidazo[4,5-b]pyridin-3-yl]-2,3-dihydro-1H-inden-1-yl]-4-(benzyloxy)-2-chloro-5-(1,3-dioxolan-2-yl)benzamide (30 mg, 0.041 mmol, 1 equiv) in TFA (2 mL) was added methanesulfonic acid (0.5 mL). The reaction mixture was stirred at room temperature for 1 h. The residue was purified by reverse-phase flash chromatography on a XBridge Shield RP 18 OBD Column (30×150 mm, 5 μm) using a 27-57% gradient of acetonitrile in water (+0.05% TFA) to afford N-[(1S)-5-[2-(2-aminopyridin-3-yl)-5-phenylimidazo[4,5-b]pyridin-3-yl]-2,3-dihydro-1H-inden-1-yl]-2-chloro-5-formyl-4-hydroxybenzamide (Example 34) (2.8 mg, 11% yield) as a light yellow solid. MS (ESI) calculated for C₃₄H₂₅ClN₆O₃: 600.17 m/z, found 601.20 [M+H]⁺. ¹H NMR (400 MHz, DMSO-d₆) δ ppm: 9.10-10.35 (m, 1H), 8.25-8.27 (m, 1H), 7.96-8.02 (m, 4H), 7.70-7.72 (m, 1H), 7.35-7.49 (m, 6H), 7.22-7.24 (m, 1H), 6.79-6.81 (m, 1H), 6.42-6.45 (m, 1H), 5.52-5.56 (m, 1H), 3.07-3.08 (m, 1H), 2.95-3.07 (m, 1H), 2.66-2.67 (m, 1H), 2.17-2.22 (m, 1H).

### Example 35: N-[(1S)-5-[2-(2-aminopyridin-3-yl)-5-phenylimidazo[4,5-b]pyridin-3-yl]-2,3-dihydro-1H-inden-1-yl]-7-formyl-1H-indazole-5-carboxamide

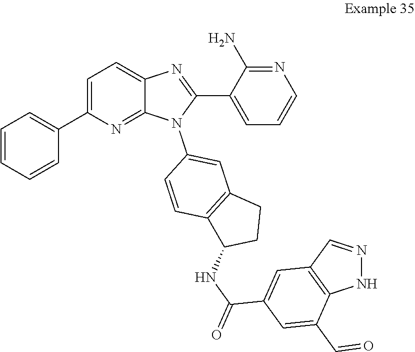 [SMILES: Nc1ncccc1-c1nc2ccc(-c3ccccc3)nc2n1-c1ccc2c(c1)CC[C@@H]2NC(=O)c1cc(C=O)c2[nH]ncc2c1]

Example 35 was prepared in a manner analogous to Example 12 using Intermediate 35-1 in place of Intermediate 12-1. MS (ESI) calculated for C₃₅H₂₆N₈O₂: 590.22 m/z, found 591.20 [M+H]⁺. ¹H NMR (400 MHz, DMSO-d₆) δ 13.90 (s, 1H), 10.25 (s, 1H), 9.17-9.18 (m, 1H), 8.79-8.83 (m, 1H), 8.61-8.68 (m, 1H), 8.39-8.44 (s, 1H), 8.26-8.37 (m, 1H), 8.00-8.04 (m, 4H), 7.40-7.48 (m, 8H), 6.97-6.99 (m, 1H), 6.46-6.47 (m, 1H), 5.72-5.73 (m, 1H), 3.33-3.36 (m, 2H), 2.90-2.96 (m, 1H), 2.29-2.30 (m, 1H).

### Intermediate 35-1: 7-(1,3-dioxolan-2-yl)-1-((2-(trimethylsilyl)ethoxy)methyl)-1H-indazole-5-carboxylic acid

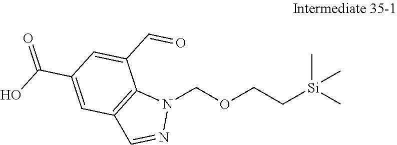 [SMILES: C[Si](C)(C)CCOCn1ncc2cc(C(=O)O)cc(C=O)c21]

### Synthetic Route:

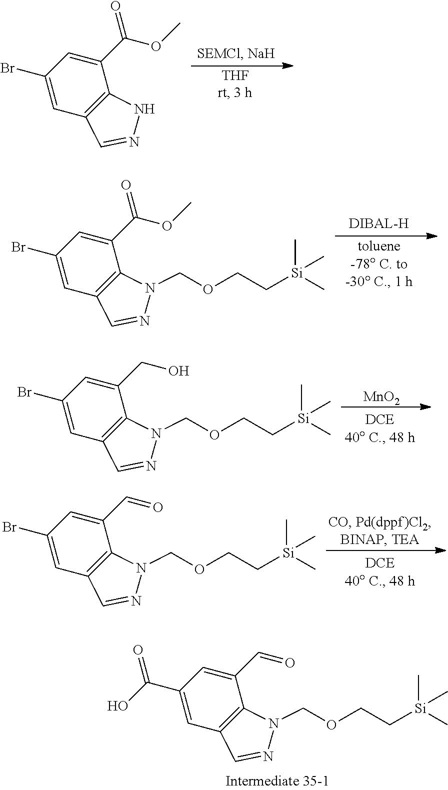

### Step 1: methyl 5-bromo-1-((2-(trimethylsilyl) ethoxy) methyl)-1H-indazole-7-carboxylate

Methyl 5-bromo-1H-indazole-7-carboxylate (756 mg, 2.96 mmol, 1 equiv) was dissolved in tetrahydrofuran (2 mL) and sodium hydride (60% dispersion in mineral oil, 142 mg, 3.56 mmol, 1.2 equiv) was added. The resulting mixture was stirred for 30 min at room temperature. [2-(chloromethoxy) ethyl]trimethylsilane (642 mg, 3.85 mmol, 1.3 equiv) was added and the mixture was stirred for 3 h at room temperature. The reaction was quenched with water (100 mL) and extracted with ethyl acetate (100 mL×3). The combined organic layers were washed with brine (50 mL), dried over anhydrous sodium sulfate, filtered and concentrated in vacuo. The resulting residue was purified by silica gel column chromatography using an eluent of 25% ethyl acetate in petroleum ether to afford methyl 5-bromo-1-{[2-(trimethylsilyl) ethoxy]methyl}indazole-7-carboxylate (600 mg, 53%) as an off-white solid. MS (ESI) calculated for C₁₅H₂₁BrN₂O₃Si: 384.05 m/z, found 386.95 [M+2H]⁺.

### Step 2: (5-bromo-1-((2-(trimethylsilyl) ethoxy) methyl)-1H-indazol-7-yl) methanol

Methyl 5-bromo-1-{[2-(trimethylsilyl) ethoxy]methyl}indazole-7-carboxylate (4.3 g, 11.2 mmol, 1 equiv) was dissolved in toluene (20 mL) and cooled to −78° C. DIBAL-H (1.5 M in toluene, 22 mL, 3 equiv) was added and the resulting solution was stirred at −30° C. for 1 h. The reaction was quenched with methanol (30 mL). Saturated aqueous potassium sodium tartrate (100 mL) was added to the mixture and stirring was continued for 30 min at room temperature. The resulting mixture was extracted with ethyl acetate (200 mL×3). The combined organic layers were washed with brine (100 mL), dried over anhydrous sodium sulfate, filtered and concentrated in vacuo. The residue was purified by silica gel column chromatography using an eluent of 25% ethyl acetate in petroleum ether to afford (5-bromo-1-{[2-(trimethylsilyl) ethoxy]methyl}indazol-7-yl) methanol (2.6 g, 59%) as a yellow oil. MS (ESI) calculated for Cl₄H₂₁BrN₂O₂Si: 356.06 m/z, found 358.90 [M+2H]⁺.

### Step 3: 5-bromo-1-((2-(trimethylsilyl) ethoxy) methyl)-1H-indazole-7-carbaldehyde

(5-bromo-1-{[2-(trimethylsilyl) ethoxy]methyl}indazol-7-yl) methanol (2.6 g, 7.3 mmol, 1 equiv) was dissolved in 1,2-dichloroethane (50 mL) and manganese(IV) oxide (12.65 g, 145.5 mmol, 20 equiv) was added. The resulting mixture was stirred at 40° C. for 48 h. The reaction mixture was filtered, rinsing with dichloromethane. The filtrate was concentrated under reduced pressure and the resulting residue was purified by silica gel column chromatography using an eluent of dichloromethane/methanol (10:1) to afford 5-bromo-1-{[2-(trimethylsilyl) ethoxy]methyl}indazole-7-carbaldehyde (1.3 g, 47%) as a light-yellow oil. MS (ESI) calculated for Cl₄H₁₉BrN₂O₂Si: 354.04 m/z, found 354.90 [M+H]⁺.

### Step 4: Synthesis of 7-formyl-1-{[2-(trimethylsilyl) ethoxy]methyl}indazole-5-carboxylic acid (Intermediate 35-1)

A pressure tank was charged with 5-bromo-1-{[2-(trimethylsilyl) ethoxy]methyl} indazole-7-carbaldehyde (1 g, 2.8 mmol, 1 equiv), Pd(dppf)Cl₂·CH₂Cl₂ (0.23 g, 0.28 mmol, 0.1 equiv), triethylamine (1.42 g, 14.1 mmol, 5 equiv), (2,2′-bis(diphenylphosphino)-1,1′-binaphthyl) (0.18 g, 0.28 mmol, 0.1 equiv) and acetonitrile (100 mL). The mixture was pressurized to 20 atm with carbon monoxide at 100° C. overnight. The reaction mixture was cooled to room temperature and filtered. The filtrate was brought to pH=6 with HCl (2N, aqueous) The resulting mixture was concentrated under reduced pressure and purified by reverse-phase flash chromatography on C18 silica gel using a 30-50% gradient of acetonitrile in water to afford 7-formyl-1-{[2-(trimethylsilyl) ethoxy]methyl}indazole-5-carboxylic acid (Intermediate 35-1) (400 mg, 41%) as a yellow oil. MS (ESI) calculated for C₁₅H₂₀N₂O₄Si: 320.05 m/z, found 319.05 [M+H]⁺.

### Example 36: N-[(1S)-5-[2-(2-aminopyridin-3-yl)-5-phenylimidazo[4,5-b]pyridin-3-yl]-2,3-dihydro-1H-inden-1-yl]-3-formyl-4-hydroxy-5-methoxybenzamide

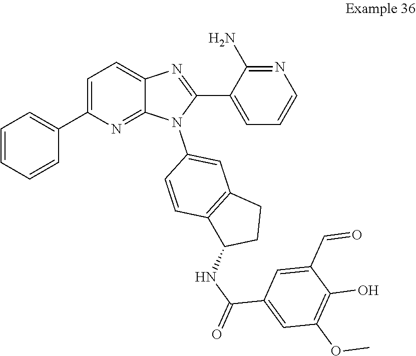 [SMILES: COc1cc(C(=O)N[C@H]2CCc3cc(-n4c(-c5cccnc5N)nc5ccc(-c6ccccc6)nc54)ccc32)cc(C=O)c1O]

Example 36 was prepared in a manner analogous to Example 12 using Intermediate 36-1 in place of Intermediate 12-1. MS (ESI) calculated for C₃₅H₂₈N₆O₄: 596.21 m/z, found 597.20 [M+H]⁺. ¹H NMR (400 MHz, DMSO-d₆) δ (ppm): 10.31 (s, 1H), 8.31-8.33 (m, 1H), 8.02-8.07 (m, 4H), 8.01 (s, 1H), 7.76-7.94 (m, 2H), 7.41-7.50 (m, 4H), 7.35-7.39 (m, 2H), 6.79-6.82 (m, 1H), 5.62-5.66 (m, 1H), 3.91 (s, 3H), 3.01-3.07 (m, 1H), 2.87-2.95 (m, 1H), 2.50-2.55 (m, 1H), 2.13-2.16 (m, 1H).

### Intermediate 36-1: 2-(2-(benzyloxy)-5-bromo-3-methoxyphenyl)-1,3-dioxolane

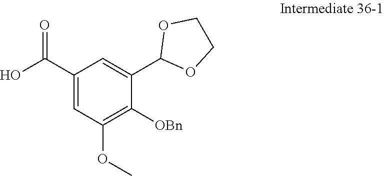 [SMILES: COc1cc(C(=O)O)cc(C2OCCO2)c1OCc1ccccc1]

### Synthetic Route:

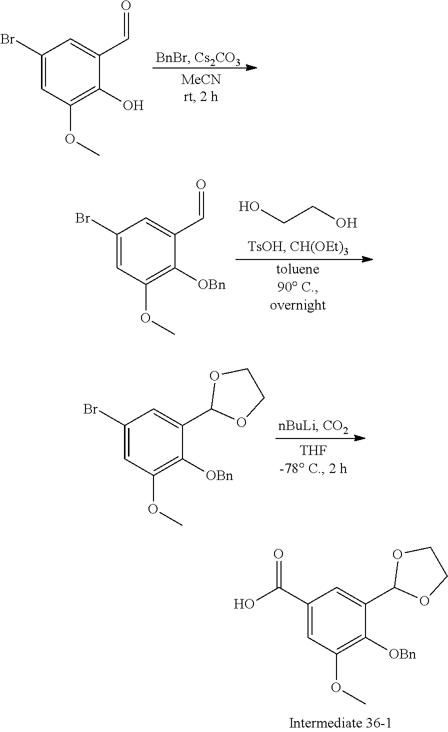

### Step 1: Synthesis of 2-(benzyloxy)-5-bromo-3-methoxybenzaldehyde

To a solution of 5-bromo-2-hydroxy-3-methoxybenzaldehyde (2.5 g, 10.8 mmol) in acetonitrile (30 mL) were added benzyl bromide (2.78 g, 16.2 mmol) and cesium carbonate (7.05 g, 21.6 mmol). The resulting mixture was stirred at room temperature for 2 h. Water was added, and the resulting mixture was extracted with ethyl acetate (30 mL×3). The combined organic layers were dried over anhydrous sodium sulfate, filtered, and concentrated in vacuo. The crude residue was triturated with petroleum ether and filtered to provide 2-(benzyloxy)-5-bromo-3-methoxybenzaldehyde as a yellow solid (3.19 g, 92% yield). MS (ESI) calculated for C₁₅H₁₃BrO₃: 320.00 m/z, found 321.10 [M+H]⁺.

### Step 2: Synthesis of 2-(2-(benzyloxy)-5-bromo-3-methoxyphenyl)-1,3-dioxolane

To a mixture of 2-(benzyloxy)-5-bromo-3-methoxybenzaldehyde (3.19 g, 9.93 mmol), ethylene glycol (4.93 g, 79.5 mmol), p-toluenesulfonic acid (0.17 g, 0.99 mmol) and triethyl orthoformate (11.78 g, 79.5 mmol) was added toluene (31 mL). The resulting mixture was stirred overnight at 90° C. Water (30 mL) was added and the resulting mixture was extracted with ethyl acetate (30 mL×3). The combined organic layers were dried over anhydrous sodium sulfate, filtered and concentrated in vacuo. The resulting residue was purified by silica gel column chromatography using an eluent of 5.7% ethyl acetate in petroleum ether to provide 2-(2-(benzyloxy)-5-bromo-3-methoxyphenyl)-1,3-dioxolane as a white solid (2.32 g, 63% yield). MS (ESI) calculated for C₁₇H₁₇BrO₄: 364.03 m/z, found 365.10 [M+H]⁺.

### Step 3: Synthesis of 4-(benzyloxy)-3-(1,3-dioxolan-2-yl)-5-methoxybenzoic acid (Intermediate 36-1)

2-[2-(benzyloxy)-5-bromo-3-methoxyphenyl]-1,3-dioxolane (2.32 g, 6.27 mmol) was suspended in tetrahydrofuran and cooled to −78° C. n-butyllithium (2.5M in hexanes, 6.28 mL, 15.7 mmol, 2.5 equiv) was added and the resulting mixture was stirred for 1 h at −78° C. Stirring was continued at −78° C. under carbon dioxide atmosphere for 2 h. The resulting mixture was concentrated to dryness to give the 4-(benzyloxy)-3-(1,3-dioxolan-2-yl)-5-methoxybenzoic acid (Intermediate 36-1) as a white semi-solid (3.88 g, crude), which was used directly in subsequent transformations without purification. MS (ESI) calculated for C₁₈H₁₈O₆: 330.11 m/z, found 331.10 [M+H]⁺.

### Example 37: (S)—N-(5-(2-(2-aminopyridin-3-yl)-5-bromo-3H-imidazo[4,5-b]pyridin-3-yl)-2,3-dihydro-1H-inden-1-yl)-3-formyl-4-hydroxybenzamide

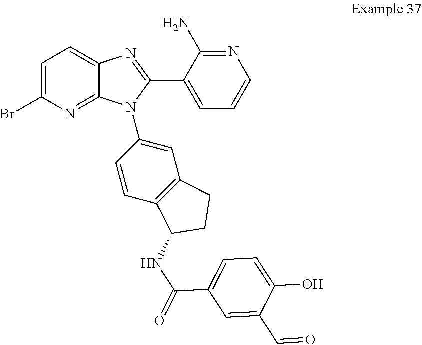 [SMILES: Nc1ncccc1-c1nc2ccc(Br)nc2n1-c1ccc2c(c1)CC[C@@H]2NC(=O)c1ccc(O)c(C=O)c1]

Example 37 was prepared in a manner analogous to Example 5 using Intermediate 37-1 in place of Intermediate 1-3. MS (ESI) calculated for C₂₈H₂₁BrN₆O₃: 568.09 m/z, found 569.00 [M+H]⁺. ¹H NMR (300 MHz, DMSO-d₆) δ (ppm): 10.31 (m, 1H), 8.94-8.97 (m, 1H), 8.30-8.40 (m, 1H), 8.10-8.15 (m, 1H), 8.08-8.09 (m, 1H), 7.99-8.00 (m, 1H), 7.55-7.57 (m, 1H), 7.27-7.48 (m, 4H), 7.04-7.07 (m, 1H), 6.92-6.93 (m, 2H), 6.43-6.44 (m, 1H), 5.61-5.64 (m, 1H), 3.01-3.08 (m, 2H), 2.31-2.32 (m, 1H), 2.06-2.07 (m, 1H).

### Intermediate 37-1: (S)-3-(3-(1-amino-2,3-dihydro-1H-inden-5-yl)-5-bromo-3H-imidazo[4,5-b]pyridin-2-yl)pyridin-2-amine

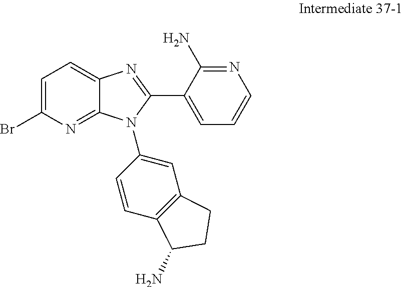 [SMILES: Nc1ncccc1-c1nc2ccc(Br)nc2n1-c1ccc2c(c1)CC[C@@H]2N]

### Synthetic Route:

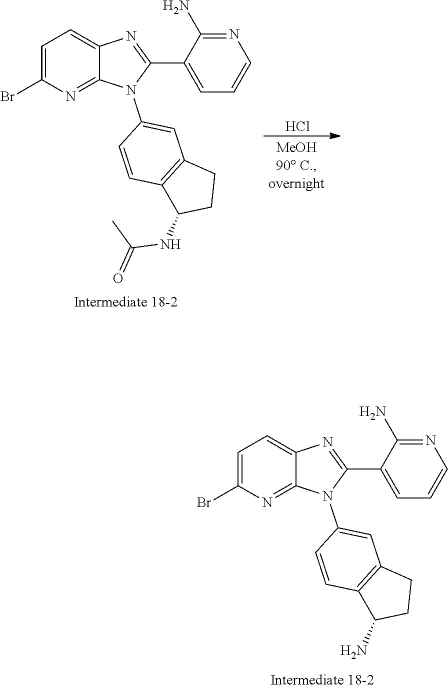 [SMILES: CC(=O)N[C@H]1CCc2cc(-n3c(-c4cccnc4N)nc4ccc(Br)nc43)ccc21 ; Cl ; Nc1ncccc1-c1nc2ccc(Br)nc2n1-c1ccc2c(c1)CC[C@@H]2N]

### Step 1: Synthesis of (S)-3-(3-(1-amino-2,3-dihydro-1H-inden-5-yl)-5-bromo-3H-imidazo[4,5-b]pyridin-2-yl)pyridin-2-amine (Intermediate 37-1)

N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (110 mg, 0.24 mmol, 1 equiv) was dissolved in HCl (2 mL, concentrated) and MeOH (2 mL). The resulting mixture was stirred overnight at 90° C. and then concentrated under reduced pressure to provide crude (S)-3-(3-(1-amino-2,3-dihydro-1H-inden-5-yl)-5-bromo-3H-imidazo[4,5-b]pyridin-2-yl)pyridin-2-amine (Intermediate 37-1) as the HCl salt (120 mg, 120% crude) which was used directly in subsequent steps without purification. MS (ESI) calculated for C₂₀H₁₇BrN₆: 420.07 m/z, found 421.20 [M+H]⁺.

### Example 38: (S)—N-(5-(2-(2-aminopyridin-3-yl)-5-(4-methylpiperazin-1-yl)-3H-imidazo[4,5-b]pyridin-3-yl)-2,3-dihydro-1H-inden-1-yl)-3-formyl-4-hydroxybenzamide

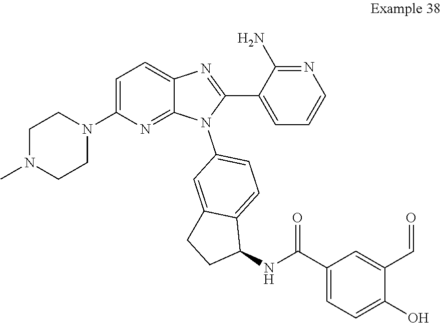 [SMILES: CN1CCN(c2ccc3nc(-c4cccnc4N)n(-c4ccc5c(c4)CC[C@@H]5NC(=O)c4ccc(O)c(C=O)c4)c3n2)CC1]

Example 38 was prepared in a manner analogous to Example 19 (starting from step 2 of that route) using Intermediate 38-1 in place of Intermediate 19-1. MS (ESI) calculated for C₃₃H₃₂N₈O₃: 588.26 m/z, found 589.25 [M+H]⁺. ¹H NMR (400 MHz, DMSO-d₆) δ 10.27 (s, 1H), 8.25-8.26 (m, 1H), 8.07-8.09 (m, 2H), 7.96-8.05 (m, 1H), 7.54-7.56 (m, 1H), 7.31-7.32 (m, 2H), 7.23-7.25 (m, 1H), 7.04-7.08 (m, 2H), 6.73-6.75 (m, 1H), 5.54-5.58 (m, 1H), 4.29-4.31 (m, 2H), 3.81-3.84 (m, 2H), 2.88-3.07 (m, 5H), 2.84-2.86 (m, 4H), 2.50-2.51 (m, 1H), 2.07-2.10 (m, 1H).

### Intermediate 38-1 N-[(1S)-5-{[6-(4-methylpiperazin-1-yl)-3-nitropyridin-2-yl]amino}-2,3-dihydro-1H-inden-1-yl]acetamide

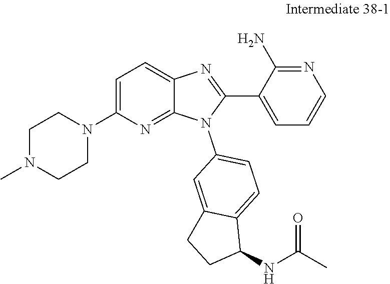 [SMILES: CC(=O)N[C@H]1CCc2cc(-n3c(-c4cccnc4N)nc4ccc(N5CCN(C)CC5)nc43)ccc21]

### Synthetic Route:

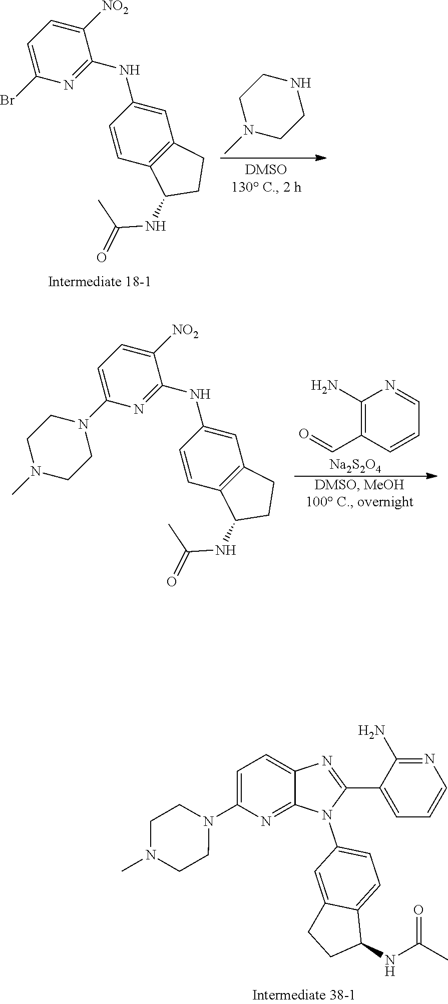

### Step 1: Synthesis of N-[(1S)-5-{[6-(4-methylpiperazin-1-yl)-3-nitropyridin-2-yl]amino}-2,3-dihydro-1H-inden-1-yl]acetamide

A solution of N-[(1S)-5-[(6-bromo-3-nitropyridin-2-yl)amino]-2,3-dihydro-H-inden-1-yl]acetamide (Intermediate 18-1) (500 mg, 1.28 mmol, 1 equiv) and 1-methylpiperazine (1.28 g, 12.8 mmol, 10 equiv) in DMSO (8 mL) was stirred at 130° C. for 2 h under N₂ atmosphere. The reaction mixture was purified using reverse-phase column chromatography on C18 silica gel using a gradient of acetonitrile in water (+0.05% ammonium bicarbonate) to provide N-[(1S)-5-{[6-(4-methylpiperazin-1-yl)-3-nitropyridin-2-yl]amino}-2,3-dihydro-1H-inden-1-yl]acetamide (400 mg, 76%) as a yellow solid. MS (ESI) calculated for C₂₁H₂₆N₆O₃: 410.21 m/z, found 411.20 [M+H]⁺.

### Step 2: Synthesis of N-[(1S)-5-[2-(2-aminopyridin-3-yl)-5-(4-methylpiperazin-1-yl)imidazo[4,5-b]pyridin-3-yl]-2,3-dihydro-1H-inden-1-yl]acetamide (Intermediate 38-1)

A mixture of N-[(1S)-5-{[6-(4-methylpiperazin-1-yl)-3-nitropyridin-2-yl]amino}-2,3-dihydro-1H-inden-1-yl]acetamide (350 mg, 0.85 mmol, 1 equiv), 2-aminopyridine-3-carbaldehyde (156 mg, 1.28 mmol, 1.5 equiv) and sodium dithionite (1.04 g, 5.97 mmol, 7 equiv) in DMSO (9 mL) and methanol (1.5 mL) was stirred at 100° C. overnight. The resulting mixture was purified by reverse-phase column chromatography on C18 silica gel using a gradient of acetonitrile in water (+0.05% ammonium bicarbonate) to provide N-[(1S)-5-[2-(2-aminopyridin-3-yl)-5-(4-methylpiperazin-1-yl)imidazo[4,5-b]pyridin-3-yl]-2,3-dihydro-1H-inden-1-yl]acetamide (Intermediate 38-1) (230 mg, 47%) as brown solid. MS (ESI) calculated for C₂₇H₃₀N₈O: 482.25 m/z, found 483.25 [M+H]⁺.

### Example 39: 1-{[(1S)-5-[2-(2-aminopyridin-3-yl)-5-phenylimidazo[4,5-b]pyridin-3-yl]-2,3-dihydro-1H-inden-1-yl]amino}-6-hydroxyisoquinoline-5-carbaldehyde

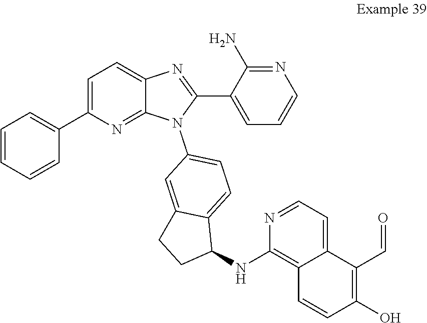 [SMILES: Nc1ncccc1-c1nc2ccc(-c3ccccc3)nc2n1-c1ccc2c(c1)CC[C@@H]2Nc1nccc2c(C=O)c(O)ccc12]

### Synthetic Route:

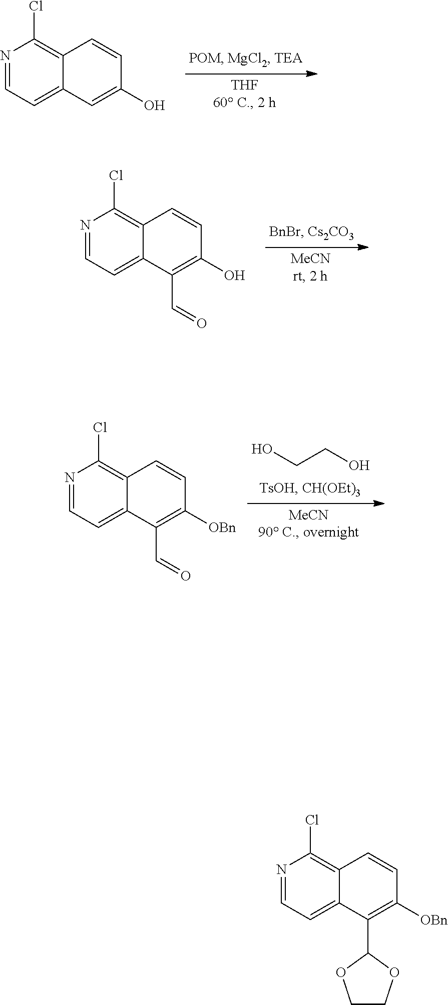

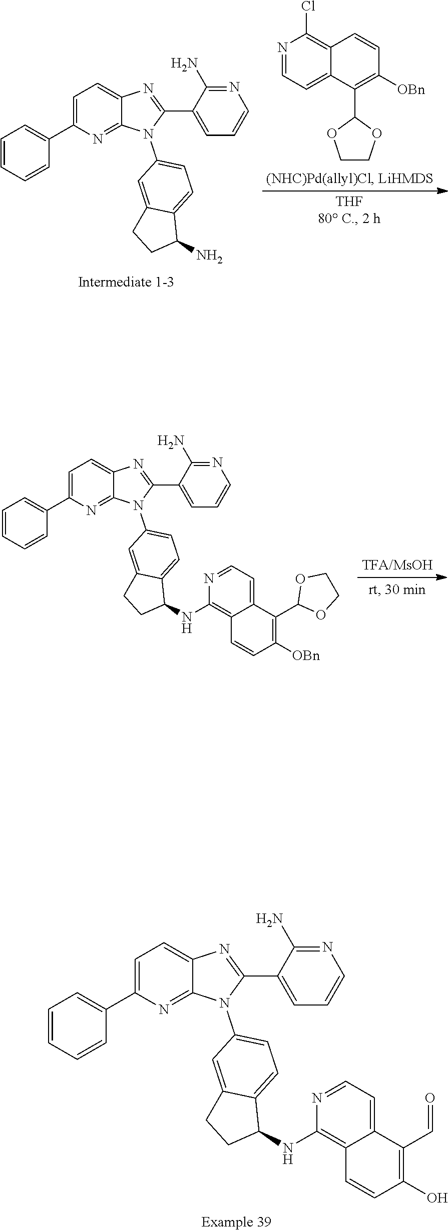 [SMILES: Nc1ncccc1-c1nc2ccc(-c3ccccc3)nc2n1-c1ccc2c(c1)CC[C@@H]2N ; Clc1nccc2c(C3OCCO3)c(OCc3ccccc3)ccc12 ; Nc1ncccc1-c1nc2ccc(-c3ccccc3)nc2n1-c1ccc2c(c1)CC[C@@H]2Nc1nccc2c(C3OCCO3)c(OCc3ccccc3)ccc12 ; Nc1ncccc1-c1nc2ccc(-c3ccccc3)nc2n1-c1ccc2c(c1)CC[C@@H]2Nc1nccc2c(C=O)c(O)ccc12]

### Step 1: Synthesis of 1-chloro-6-hydroxyisoquinoline-5-carbaldehyde

To a mixture of 1-chloroisoquinolin-6-ol (1 g, 5.57 mmol), polyoxymethylene (2.51 g, 27.8 mmol), magnesium(II) chloride (0.8 g, 8.35 mmol) and triethylamine (1.41 g, 13.9 mmol) was added tetrahydrofuran (15 mL) and the resulting mixture was stirred at 60° C. for 2 h. The resulting mixture was concentrated to dryness and the residue was purified by reverse-phase column chromatography on C18 silica gel using a 10-50% gradient of acetonitrile in water (+10 mmol/L ammonium bicarbonate) to afford 1-chloro-6-hydroxyisoquinoline-5-carbaldehyde (200 mg, 19%) as a white solid. MS (ESI) calculated for C₁₀H₆ClNO₂: 207.00 m/z, found 208.10 [M+H]⁺.

### Step 2: Synthesis of 6-(benzyloxy)-1-chloroisoquinoline-5-carbaldehyde

To a solution of 1-chloro-6-hydroxyisoquinoline-5-carbaldehyde (1 g, 4.8 mmol) in acetonitrile (15 mL) were added benzyl bromide (1.24 g, 7.23 mmol) and cesium carbonate (3.14 g, 9.63 mmol). The mixture was stirred at room temperature for 2 h. Water was added, and the resulting mixture was extracted with ethyl acetate (30 mL×3). The combined organic layers were dried over anhydrous sodium sulfate, filtered, and concentrated in vacuo. Petroleum ether (50 mL) was added, and the mixture was stirred for 20 minutes. The resulting mixture was filtered and concentrated to dryness to provide 6-(benzyloxy)-1-chloroisoquinoline-5-carbaldehyde as an off-white solid (180 mg, 13% yield). MS (ESI) calculated for C₁₇H₁₂ClNO₂: 297.05 m/z, found 298.10 [M+H]⁺.

### Step 3: Synthesis of 6-(benzyloxy)-1-chloro-5-(1,3-dioxolan-2-yl)isoquinoline

A mixture of 6-(benzyloxy)-1-chloroisoquinoline-5-carbaldehyde (688 mg, 2.31 mmol), ethylene glycol (1.15 g, 18.5 mmol), p-toluenesulfonic acid (40 mg, 0.23 mmol), triethyl orthoformate (2.74 mg, 18.5 mmol) and toluene (10 mL) was stirred at 90° C. overnight. Water (40 mL) was added, and the mixture was extracted with ethyl acetate (30 mL×3). The combined organic layers were dried over anhydrous sodium sulfate, filtered and concentrated in vacuo. The resulting residue was purified by reverse-phase column chromatography on C18 silica gel using an eluent of 60% acetonitrile in water to provide 6-(benzyloxy)-1-chloro-5-(1,3-dioxolan-2-yl)isoquinoline (268 mg, 32% yield). MS (ESI) calculated for C₁₉H₁₆ClNO₃: 341.08 m/z, found 342.15 [M+H]⁺.

### Step 4: Synthesis of N-[(1S)-5-[2-(2-aminopyridin-3-yl)-5-phenylimidazo[4,5-b]pyridin-3-yl]-2,3-dihydro-1H-inden-1-yl]-6-(benzyloxy)-5-(1,3-dioxolan-2-yl)isoquinolin-1-amine

To a mixture of 6-(benzyloxy)-1-chloro-5-(1,3-dioxolan-2-yl)isoquinoline (150 mg, 0.42 mmol), 3-{3-[(1S)-1-amino-2,3-dihydro-1H-inden-5-yl]-5-phenylimidazo[4,5-b]pyridin-2-yl}pyridin-2-amine (Intermediate 1-3) (211 mg, 0.50 mmol), (NHC)Pd(allyl)Cl (CAS: 478980-03-9) (72 mg, 0.13 mmol) and lithium bis(trimethylsilyl)amide (281 mg, 1.68 mmol) was added tetrahydrofuran (5 mL) and the resulting mixture was stirred under N₂ at 80° C. for 2 h. The resulting mixture was concentrated to dryness and purified by reverse-phase column chromatography on C18 silica gel using an eluent of 80% acetonitrile in water to provide N-[(1S)-5-[2-(2-aminopyridin-3-yl)-5-phenylimidazo[4,5-b]pyridin-3-yl]-2,3-dihydro-1H-inden-1-yl]-6-(benzyloxy)-5-(1,3-dioxolan-2-yl)isoquinolin-1-amine as a yellow solid (53 mg, 17% yield). MS (ESI) calculated for C₄₅H₃₇N₇O₃: 723.29 m/z, found 724.10 [M+H]⁺.

### Step 5: Synthesis of 1-{[(1S)-5-[2-(2-aminopyridin-3-yl)-5-phenylimidazo[4,5-b]pyridin-3-yl]-2,3-dihydro-1H-inden-1-yl]amino}-6-hydroxyisoquinoline-5-carbaldehyde (Example 39)

A mixture of N-[(1S)-5-[2-(2-aminopyridin-3-yl)-5-phenylimidazo[4,5-b]pyridin-3-yl]-2,3-dihydro-1H-inden-1-yl]-6-(benzyloxy)-5-(1,3-dioxolan-2-yl)isoquinolin-1-amine (53 mg, 0.073 mmol, 1 equiv), TFA (3 mL) and methanesulfonic acid (1 mL) was stirred at room temperature for 30 min. The mixture was neutralized to pH 7 with saturated aqueous sodium bicarbonate. The resulting mixture was extracted with ethyl acetate (3×30 mL). The combined organic layers were dried over anhydrous sodium sulfate, filtered and concentrated in vacuo. The resulting residue was purified by Preparative HPLC on a XSelect CSH C18 Column (30×150 mm, 5 μm) using a 17-47% gradient of acetonitrile in water (+0.05% TFA) to afford 1-{[(1S)-5-[2-(2-aminopyridin-3-yl)-5-phenylimidazo[4,5-b]pyridin-3-yl]-2,3-dihydro-1H-inden-1-yl]amino}-6-hydroxyisoquinoline-5-carbaldehyde (Example 39) (4 mg, 9%) as a yellow solid. MS (ESI) calculated for C₃₆H₂₇N₇O₂: 589.22 m/z, found 590.25 [M+H]⁺. ¹H NMR (400 MHz, DMSO-d₆) δ (ppm): 10.66 (s, 1H), 8.70-8.73 (m, 1H), 8.26-8.32 (m, 2H), 8.00-8.05 (m, 4H), 7.77-7.79 (m, 1H), 7.66 (s, 1H), 7.33-7.52 (m, 7H), 6.69-6.71 (m, 1H), 5.64 (s, 1H), 3.15 (s, 1H), 2.98 (s, 1H), 2.50-2.51 (m, 1H), 2.21-2.23 (m, 1H).

### Example 40: N-[(1S)-5-[2-(2-aminopyridin-3-yl)-5-phenylimidazo[4,5-b]pyridin-3-yl]-2,3-dihydro-1H-inden-1-yl]-4-formyl-5-hydroxypyridine-2-carboxamide

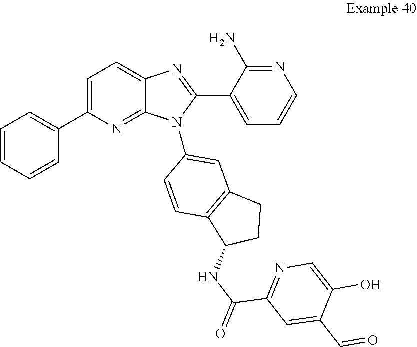 [SMILES: Nc1ncccc1-c1nc2ccc(-c3ccccc3)nc2n1-c1ccc2c(c1)CC[C@@H]2NC(=O)c1cc(C=O)c(O)cn1]

Example 40 was prepared in a manner analogous to Example 12 using Intermediate 40-1 in place of Intermediate 12-1. MS (ESI) calculated for C₃₃H₂₅N₇O₃: 567.20, found 568.20 [M+H]⁺. ¹H NMR (400 MHz, DMSO-d₆) δ ppm: 10.36 (s, 1H), 8.25-8.27 (m, 1H), 7.96-8.04 (m, 4H), 7.22-7.53 (m, 9H), 6.48-6.51 (m, 1H), 5.56-5.58 (m, 1H), 2.87-3.11 (m, 2H), 2.6-3.70 (m, 1H), 1.87-2.01 (m, 1H).

### Intermediate 40-1: 5-(benzyloxy)-4-(1,3-dioxolan-2-yl)picolinic acid

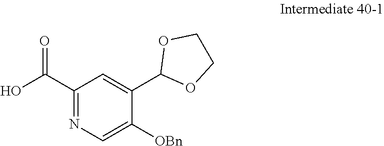 [SMILES: O=C(O)c1cc(C2OCCO2)c(OCc2ccccc2)cn1]

Intermediate 40-1 was prepared in a manner analogous to Intermediate 36-1 using 2-bromo-5-hydroxyisonicotinaldehyde in place of 5-bromo-2-hydroxy-3-methoxybenzaldehyde. The crude mixture of Intermediate 40-1 was purified by reverse-phase flash chromatography on C18 silica gel using a 10-70% gradient of acetonitrile in water to provide 5-(benzyloxy)-4-(1,3-dioxolan-2-yl)pyridine-2-carboxylic acid (Intermediate 40-1) as a white solid. MS (ESI) calculated for C₁₆H₁₅NO₅: 301.10, found 302.10 [M+H]⁺.

### Example 41: N-[(1S)-5-[2-(2-aminopyridin-3-yl)-5-phenylimidazo[4,5-b]pyridin-3-yl]-2,3-dihydro-1H-inden-1-yl]-3-formyl-4-hydroxy-5-methylbenzamide

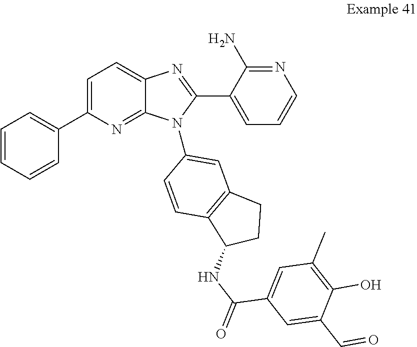 [SMILES: Cc1cc(C(=O)N[C@H]2CCc3cc(-n4c(-c5cccnc5N)nc5ccc(-c6ccccc6)nc54)ccc32)cc(C=O)c1O]

Example 41 was prepared in a manner analogous to Example 12 using Intermediate 41-1 in place of Intermediate 12-1. MS (ESI) calculated for C₃₅H₂₈N₆O₃: 580.65 m/z, found 581.30 [M+H]⁺. ¹H NMR (400 MHz, DMSO-d₆) δ (ppm): 10.08 (s, 1H), 8.31-8.32 (m, 1H), 8.29-8.30 (m, 1H), 8.00-8.07 (m, 5H), 7.66-7.67 (m, 1H), 7.39-7.50 (m, 6H), 6.72-6.74 (m, 1H), 5.63-5.64 (m, 1H), 2.92-3.12 (m, 2H), 2.50-2.54 (m, 1H), 2.34-2.36 (m, 3H), 2.09-2.25 (m, 1H).

### Intermediate 41-1: 3-(1,3-dioxolan-2-yl)-4-((4-methoxybenzyl)oxy)-5-methylbenzoic acid

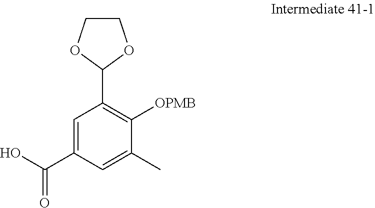

### Synthetic Route:

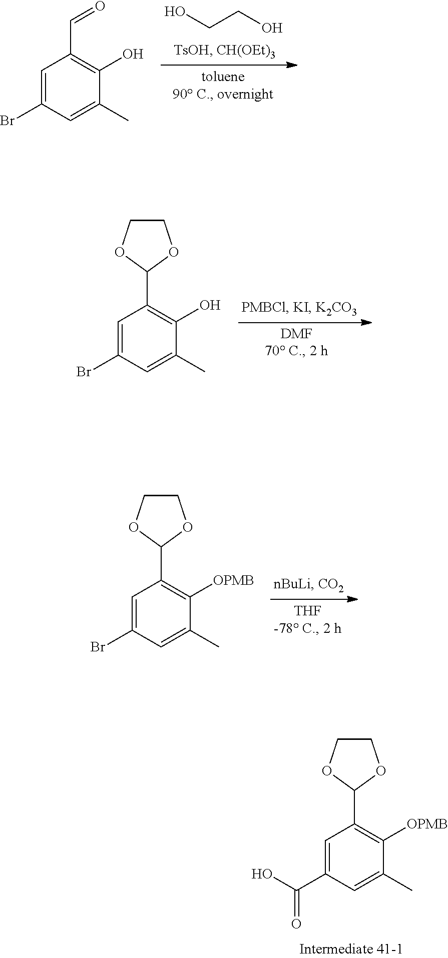

### Step 1: Synthesis of 4-bromo-2-(1,3-dioxolan-2-yl)-6-methylphenol

To a solution of 5-bromo-2-hydroxy-3-methylbenzaldehyde (1.8 g, 8.4 mmol, 1 equiv) in toluene (15 mL, 141 mmol) was added ethylene glycol (2.60 g, 41.9 mmol, 5 equiv) and p-toluenesulfonic acid (0.14 g, 0.84 mmol, 0.1 equiv) The resulting mixture was stirred at 90° C. overnight. The reaction mixture was then treated with water (30 mL) dropwise over 10 min and extracted with ethyl acetate. (30 mL×2). The combined organic layers were washed with brine, dried over anhydrous sodium sulfate, filtered and concentrated in vacuo. The resulting oil was then purified by silica gel column chromatography using a gradient of 0-25% ethyl acetate in petroleum ether to afford 4-bromo-2-(1,3-dioxolan-2-yl)-6-methylphenol (1.7 g, 71%) as a yellow oil. MS (ESI) calculated for C₁₀H₁₁BrO₃: 257.99 m/z, found 258.95 [M+H]⁺.

### Step 2: Synthesis of 2-(5-bromo-2-((4-methoxybenzyl)oxy)-3-methylphenyl)-1,3-dioxolane

To a solution of 4-bromo-2-(1,3-dioxolan-2-yl)-6-methylphenol (1.7 g, 6.6 mmol, 1 equiv) in N,N-dimethylformamide (15 mL) was added para-methoxybenzyl chloride (1.23 g, 7.87 mmol, 1.2 equiv), potassium iodide (0.11 g, 0.66 mmol, 0.1 equiv) and potassium carbonate (2.72 g, 19.7 mmol, 3 equiv). The resulting mixture stirred for 2 h at 70° C. under nitrogen atmosphere. The reaction mixture was then treated with water (30 mL) and extracted with ethyl acetate (30 mL×2). The combined organic layers were washed with brine, dried over anhydrous sodium sulfate, filtered and concentrated in vacuo. The resulting oil was then purified by silica gel column chromatography using a 0-25% gradient of ethyl acetate in petroleum ether to afford 2-(5-bromo-2-((4-methoxybenzyl)oxy)-3-methylphenyl)-1,3-dioxolane (1.6 g, 64%) as a white solid. MS (ESI) calculated for C₁₈H₁₉BrO₄: 378.05 m/z, found 379.05 [M+H]⁺.

### Step 3: Synthesis of 3-(1,3-dioxolan-2-yl)-4-((4-methoxybenzyl)oxy)-5-methylbenzoic acid (Intermediate 41-1)

To a cooled (−78° C.) solution of 2-(5-bromo-2-((4-methoxybenzyl)oxy)-3-methylphenyl)-1,3-dioxolane (800 mg, 2.11 mmol, 1 equiv) in tetrahydrofuran (20 mL) was added n-butyllithium (2.5M in hexanes, 2.11 mL, 5.27 mmol, 2.5 equiv). The resulting mixture was stirred at −78° C. for 1 h. Stirring at −78° C. was continued for 1 h under CO₂ atmosphere. The reaction mixture was concentrated in vacuo to provide crude 3-(1,3-dioxolan-2-yl)-4-((4-methoxybenzyl)oxy)-5-methylbenzoic acid (Intermediate 41-1) (600 mg, 82.60%), which was used directly in subsequent transformations without further purification. MS (ESI) calculated for C₁₉H₂₀O₆: 344.13 m/z, found 343.05 [M−H]⁻.

### Example 42: N-[(1S)-5-[2-(2-aminopyridin-3-yl)-5-(morpholin-4-yl)imidazo[4,5-b]pyridin-3-yl]-2,3-dihydro-1H-inden-1-yl]-3-formyl-4-hydroxybenzamide

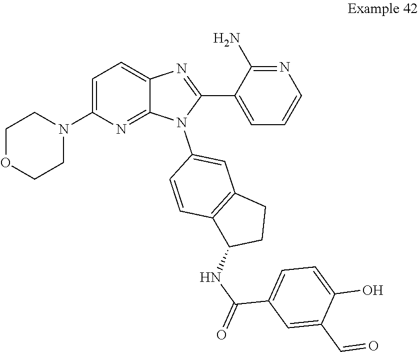 [SMILES: Nc1ncccc1-c1nc2ccc(N3CCOCC3)nc2n1-c1ccc2c(c1)CC[C@@H]2NC(=O)c1ccc(O)c(C=O)c1]

Example 42 was prepared in a manner analogous to Example 12 using Intermediate 42-1 in place of Intermediate 1-3 and Intermediate 5-1 in place of Intermediate 12-1. MS (ESI) calculated for C₃₂H₂₉N₇O₄: 575.23 m/z, found 576.30 [M+H]⁺. ¹H-NMR (400 MHz, DMSO-d₆) δ (ppm): 10.30 (s, 1H), 8.31 (s, 1H), 8.09-8.19 (m, 1H), 7.97-8.08 (m, 2H), 7.26-7.31 (m, 2H), 7.19-7.21 (m, 2H), 7.07-7.09 (m, 1H), 6.89-6.92 (m, 1H), 6.47-6.48 (m, 1H), 5.58-5.62 (m, 1H), 3.67-3.69 (m, 4H), 3.42 (s, 4H), 2.95-3.02 (m, 2H), 2.50-2.51 (m, 1H), 2.08-2.19 (m, 1H).

### Intermediate 42-1: 3-{3-[(1S)-1-amino-2,3-dihydro-1H-inden-5-yl]-5-(morpholin-4-yl) imidazo[4,5-b]pyridin-2-yl}pyridin-2-amine

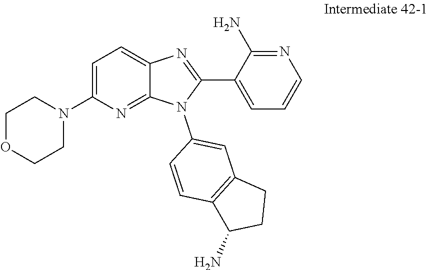 [SMILES: Nc1ncccc1-c1nc2ccc(N3CCOCC3)nc2n1-c1ccc2c(c1)CC[C@@H]2N]

### Synthetic Route:

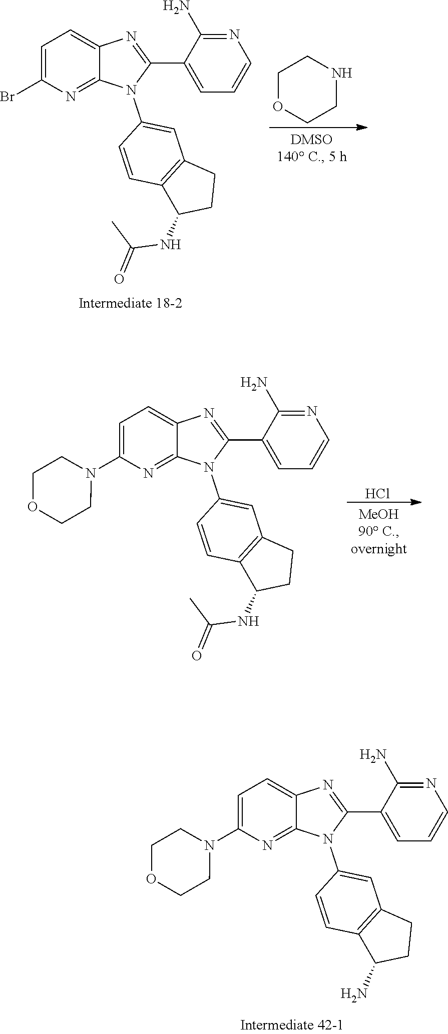

### Step 1: Synthesis of N-[(1S)-5-[2-(2-aminopyridin-3-yl)-5-(morpholin-4-yl) imidazo[4,5-b] pyridin-3-yl]-2,3-dihydro-1H-inden-1-yl]acetamide

A solution of N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (300 mg, 0.65 mmol, 1 equiv) and morpholine (564 mg, 6.47 mmol, 10 equiv) in DMSO (3 mL) was stirred for 5 h at 140° C. The crude product was purified by reverse-phase flash chromatography on C18 silica gel using a 5-95% gradient of acetonitrile in water (+0.05% ammonium bicarbonate) with a 10 minute hold at 40% acetonitrile to afford N-[(1S)-5-[2-(2-aminopyridin-3-yl)-5-(morpholin-4-yl) imidazo[4,5-b]pyridin-3-yl]-2,3-dihydro-1H-inden-1-yl]acetamide (200 mg, 63%) as a yellow solid. MS (ESI) calculated For C₂₆H₂₇N₇O₂: 469.22 m/z, found 470.25 [M+H]⁺.

### Step 2: Synthesis of 3-{3-[(1S)-1-amino-2,3-dihydro-1H-inden-5-yl]-5-(morpholin-4-yl) imidazo[4,5-b]pyridin-2-yl}pyridin-2-amine (Intermediate 42-1)

A solution of N-[(1S)-5-[2-(2-aminopyridin-3-yl)-5-(morpholin-4-yl)imidazo[4,5-b]pyridin-3-yl]-2,3-dihydro-1H-inden-1-yl]acetamide (200 mg, 0.43 mmol, 1 equiv) in HCl (3 mL, concentrated) and methanol (2 mL) was stirred for overnight at 90° C. The mixture was brought to pH 7-8 with saturated aqueous sodium bicarbonate. The resulting mixture was extracted with dichloromethane (50 mL×3). The combined organic layers were washed with water (50 mL×3), dried over anhydrous sodium sulfate, filtered and concentrated in vacuo to provide 3-{3-[(1S)-1-amino-2,3-dihydro-1H-inden-5-yl]-5-(morpholin-4-yl) imidazo[4,5-b] pyridin-2-yl}pyridin-2-amine (Intermediate 42-1) (100 mg, 47%) as a yellow solid. MS (ESI) calculated for C₂₄H₂₅N₇O: 427.21 m/z, found 428.10 [M+H]⁺.

### Example 43: (S)—N-(5-(2-(2-aminopyridin-3-yl)-5-ethyl-3H-imidazo[4,5-b]pyridin-3-yl)-2,3-dihydro-1H-inden-1-yl)-3-formyl-4-hydroxybenzamide

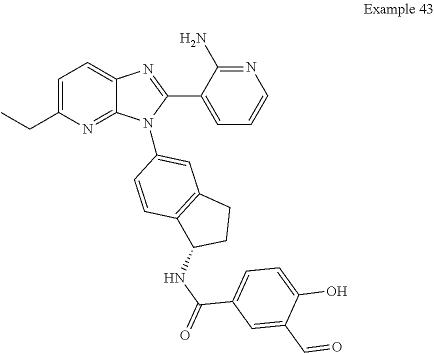 [SMILES: CCc1ccc2nc(-c3cccnc3N)n(-c3ccc4c(c3)CC[C@@H]4NC(=O)c3ccc(O)c(C=O)c3)c2n1]

Example 43 was prepared in a manner analogous to Example 12 using Intermediate 43-1 in place of Intermediate 1-3 and Intermediate 5-1 in place of Intermediate 12-1. MS (ESI) calculated for C₃₀H₂₆N₆O₃: 518.21 m/z, found 519.25 [M+H]⁺. ¹H NMR (300 MHz, DMSO-d₆) δ (ppm): 10.28 (s, 1H), 9.29-9.30 (m, 1H), 8.03-8.24 (m, 3H), 7.70-7.73 (m, 1H), 7.33-7.39 (m, 4H), 7.07-7.11 (m, 1H), 6.76-6.80 (m, 1H), 5.58-5.63 (m, 1H), 2.79-3.02 (m, 4H), 2.51-2.52 (m, 1H), 2.06-2.13 (m, 1H), 1.19-1.24 (m, 3H).

### Intermediate 43-1: (S)-3-(3-(1-amino-2,3-dihydro-1H-inden-5-yl)-5-ethyl-3H-imidazo[4,5-b]pyridin-2-yl)pyridin-2-amine

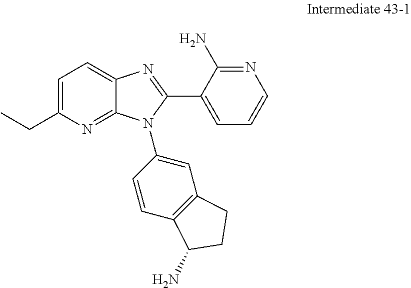 [SMILES: CCc1ccc2nc(-c3cccnc3N)n(-c3ccc4c(c3)CC[C@@H]4N)c2n1]

### Synthetic Route:

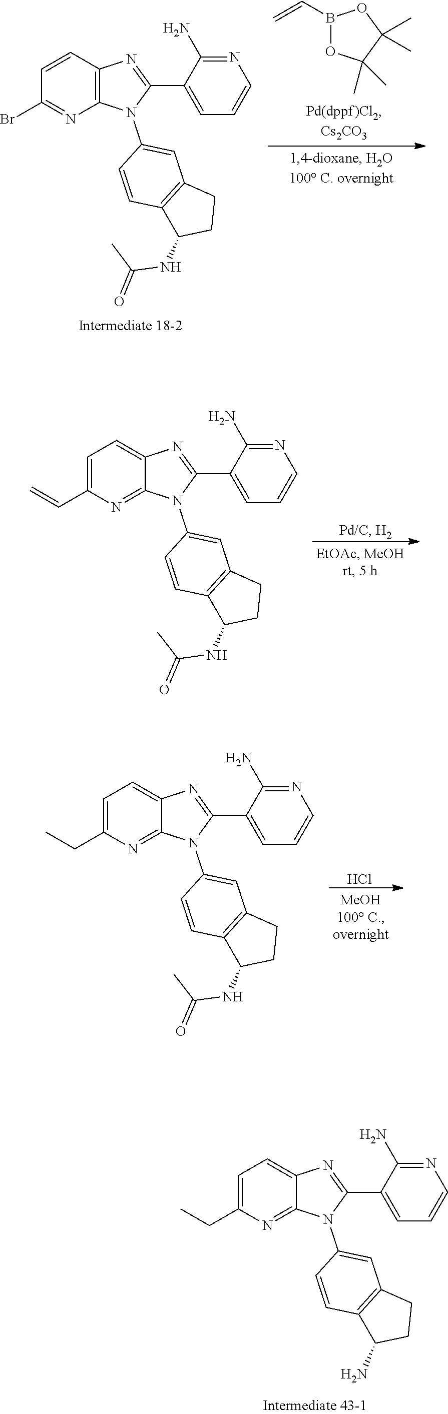

### Step 1: Synthesis of N-[(1S)-5-[2-(2-aminopyridin-3-yl)-5-ethenylimidazo[4,5-b]pyridin-3-yl]-2,3-dihydro-1H-inden-1-yl]acetamide

N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (300 mg, 0.65 mmol, 1 equiv) and 2-ethenyl-4,4,5,5-tetramethyl-1,3,2-dioxaborolane (130 mg, 0.84 mmol, 1.3 equiv) were dissolved in 1,4-dioxane (4.8 mL) and H₂O (1.2 mL). Pd(dppf)Cl₂ (53 mg, 0.065 mmol, 0.1 equiv) and cesium carbonate (422 mg, 1.29 mmol, 2 equiv) were added and the mixture was stirred overnight at 100° C. under N₂. Water was added and the resulting mixture was extracted with ethyl acetate (150 mL). The organic layer was washed with brine (100 mL), dried over anhydrous sodium sulfate, filtered and concentrated in vacuo. The resulting residue was purified by silica gel column chromatography eluting with dichloromethane/methanol (10:1) to provide N-[(1S)-5-[2-(2-aminopyridin-3-yl)-5-ethenylimidazo[4,5-b]pyridin-3-yl]-2,3-dihydro-1H-inden-1-yl]acetamide (260 mg, 90%) as a yellow solid. MS (ESI) calculated for C₂₄H₂₂N₆O: 410.19 m/z, found 411.25 [M+H]⁺.

### Step 2: Synthesis of N-[(1S)-5-[2-(2-aminopyridin-3-yl)-5-ethylimidazo[4,5-b]pyridin-3-yl]-2,3-dihydro-1H-inden-1-yl]acetamide

N-[(1S)-5-[2-(2-aminopyridin-3-yl)-5-ethenylimidazo[4, 5-b]pyridin-3-yl]-2,3-dihydro-1H-inden-1-yl]acetamide (300 mg, 0.73 mmol, 1 equiv) was dissolved in ethyl acetate (8 mL) and MeOH (10 mg). 10% Pd/C (160 mg, 0.15 mmol, 0.21 equiv) was added under nitrogen atmosphere. The mixture was stirred under hydrogen atmosphere for 5 h then filtered through Celite rinsing with ethyl acetate and methanol. The filtrate was concentrated under reduced pressure to afford N-[(1S)-5-[2-(2-aminopyridin-3-yl)-5-ethylimidazo[4,5-b]pyridin-3-yl]-2,3-dihydro-1H-inden-1-yl]acetamide (280 mg, 84%) as a yellow oil. MS (ESI) calculated for C₂₄H₂₄N₆O: 412.20 m/z, found 413.20 [M+H]⁺.

### Step 3: Synthesis of 3-{3-[(1S)-1-amino-2,3-dihydro-1H-inden-5-yl]-5-ethylimidazo[4,5-b]pyridin-2-yl}pyridin-2-amine (Intermediate 43-1)

N-[(1S)-5-[2-(2-aminopyridin-3-yl)-5-ethylimidazo[4,5-b]pyridin-3-yl]-2,3-dihydro-1H-inden-1-yl]acetamide (200 mg, 0.49 mmol) was dissolved in methanol (3 mL) and HCl (3 mL, concentrated) was added. The resulting mixture was stirred overnight at 100° C. The resulting mixture was concentrated under reduced pressure to provide crude 3-{3-[(1S)-1-amino-2,3-dihydro-1H-inden-5-yl]-5-ethylimidazo[4,5-b]pyridin-2-yl}pyridin-2-amine (Intermediate 43-1) (200 mg, 98%) which was used without purification in the subsequent transformations. MS (ESI) calculated for C₂₂H₂₂N₆: 370.19 m/z, found 371.05 [M+H]⁺.

### Example 44: (S)—N-(5-(2-(2-aminopyridin-3-yl)-5-methyl-3H-imidazo[4,5-b]pyridin-3-yl)-2,3-dihydro-1H-inden-1-yl)-3-fluoro-5-formyl-4-hydroxybenzamide

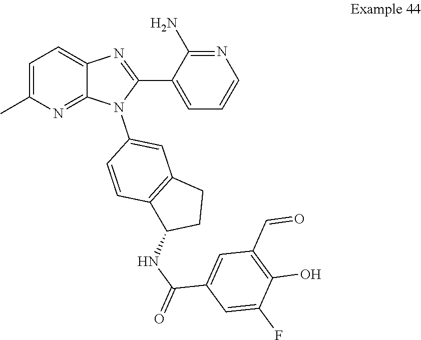 [SMILES: Cc1ccc2nc(-c3cccnc3N)n(-c3ccc4c(c3)CC[C@@H]4NC(=O)c3cc(F)c(O)c(C=O)c3)c2n1]

Example 44 was prepared in a manner analogous to Example 12 using Intermediate 13-2 in place of Intermediate 1-3 and Intermediate 44-1 in place of Intermediate 12-1. MS (ESI) calculated for C₂₉H₂₃FN₆O₃: 522.18 m/z, found 523.20 [M+H]⁺. ¹H NMR (400 MHz, DMSO-d₆) δ ppm: 10.28 (s, 1H), 9.09-9.15 (m, 1H), 8.12-8.14 (m, 1H), 8.02-8.03 (m, 1H), 7.70-7.71 (m, 1H), 7.53-7.55 (m, 1H), 7.39-7.42 (m, 2H), 7.30-7.32 (m, 2H), 7.10-7.28 (m, 1H), 6.74-6.78 (m, 1H), 5.55 (s, 1H), 2.89-2.91 (m, 2H), 2.50-2.51 (m, 1H), 2.52-2.53 (m, 3H), 2.36 (s, 1H). ¹⁹F NMR (376 MHz, DMSO-d₆) δ −137.15.

### Intermediate 44-1: 4-(benzyloxy)-3-(1,3-dioxolan-2-yl)-5-fluorobenzoic acid

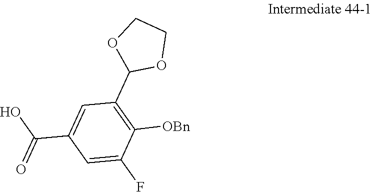 [SMILES: O=C(O)c1cc(F)c(OCc2ccccc2)c(C2OCCO2)c1]

Intermediate 44-1 was prepared in a manner analogous to Intermediate 36-1 using 5-bromo-3-fluoro-2-hydroxybenzaldehyde in place of 5-bromo-2-hydroxy-3-methoxybenzaldehyde. The crude material was purified by reverse-phase flash chromatography on C18 silica gel using a gradient of acetonitrile in water to afford 4-(benzyloxy)-3-(1,3-dioxolan-2-yl)-5-fluorobenzoic acid (Intermediate 44-1) as a yellow solid. MS (ESI) calculated for C₁₇H₁₅FO₅: 318.09 m/z, found 319.05 [M+H]⁺.

### Example 45: N-[(1S)-5-[2-(2-aminopyridin-3-yl)-5-methylimidazo[4,5-b]pyridin-3-yl]-2,3-dihydro-1H-inden-1-yl]-4-acetamido-3-formylbenzamide

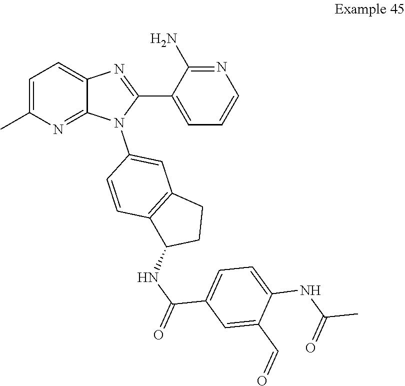 [SMILES: CC(=O)Nc1ccc(C(=O)N[C@H]2CCc3cc(-n4c(-c5cccnc5N)nc5ccc(C)nc54)ccc32)cc1C=O]

Example 45 was prepared in a manner analogous to Example 12 using Intermediate 13-2 in place of Intermediate 1-3 and Intermediate 45-1 in place of Intermediate 12-1. MS (ESI) calculated for C₃₁H₂₇N₇O₃: 545.22 m/z, found 546.25 [M+H]⁺. ¹H NMR (400 MHz, DMSO-d₆) δ (ppm): 10.01 (s, 1H), 8.45-8.46 (m, 1H), 8.21-8.26 (m, 2H), 8.11-8.19 (m, 1H), 8.01-8.09 (m, 1H), 7.50-7.52 (m, 1H), 7.36-7.38 (m, 2H), 7.23-7.29 (m, 2H), 6.61-6.64 (m, 1H), 5.62-5.66 (m, 1H), 3.05-3.06 (m, 1H), 2.92-2.94 (m, 1H), 2.56-2.57 (m, 1H), 2.54-2.55 (m, 3H), 2.32-2.33 (m, 3H), 2.11-2.19 (m, 1H).

### Intermediate 45-1: 4-acetamido-3-(1,3-dioxolan-2-yl)benzoic acid

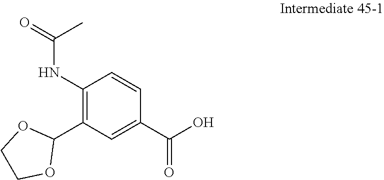 [SMILES: CC(=O)Nc1ccc(C(=O)O)cc1C1OCCO1]

### Synthetic Route:

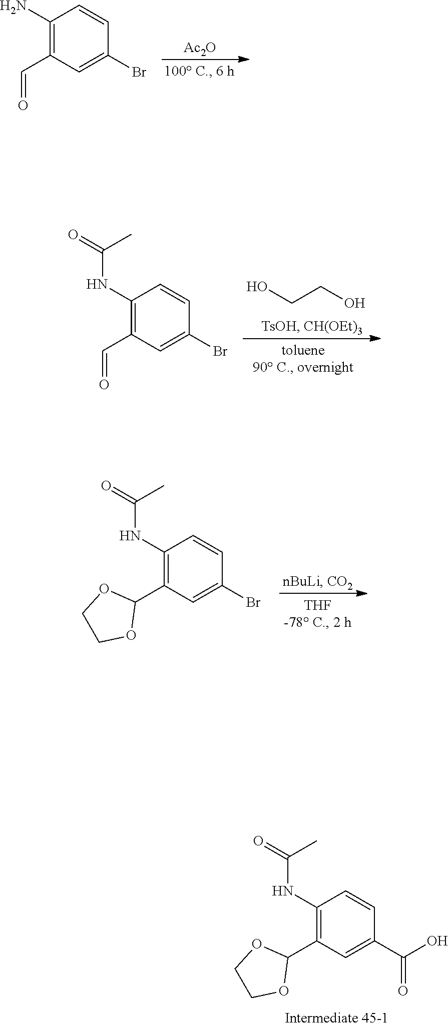

### Step 1: Synthesis of N-(4-bromo-2-formylphenyl)acetamide

A solution of 2-amino-5-bromobenzaldehyde (1 g, 5 mmol) in acetic anhydride (20 mL) was stirred at 100° C. for 6 h. The reaction mixture was filtered to afford crude N-(4-bromo-2-formylphenyl)acetamide (1 g, 80%) as a white solid, which was used directly in the next step without purification. MS (ESI) calculated for C₉H₈BrNO₂: 240.97 m/z, found 241.90 [M+H]⁺.

### Step 2: Synthesis of N-[4-bromo-2-(1,3-dioxolan-2-yl)phenyl]acetamide

To a solution of N-(4-bromo-2-formylphenyl)acetamide (1 g, 4.13 mmol, 1 equiv) in toluene (15 mL) was added ethylene glycol (1.28 g, 20.7 mmol, 5 equiv), triethyl orthoformate (1.84 g, 12.39 mmol, 3 equiv) and p-toluenesulfonic acid (0.07 g, 0.41 mmol, 0.1 equiv). The resulting mixture was stirred at 90° C. overnight. The reaction mixture was concentrated in vacuo and the resulting residue was purified by reverse-phase flash chromatography on C18 silica gel using a 20-95% gradient of acetonitrile in water (+0.05% ammonium bicarbonate) with a 10 minute hold a 70% acetonitrile to afford N-[4-bromo-2-(1,3-dioxolan-2-yl)phenyl]acetamide (500 mg, 41%) as a white solid. MS (ESI) calculated for C₁₁H₁₂BrNO₃: 285.00 m/z, found 283.90 [M−H]⁻.

### Step 3: Synthesis of 3-(1,3-dioxolan-2-yl)-4-acetamidobenzoic acid (Intermediate 45-1)

To a cooled (−78° C.) solution of N-[4-bromo-2-(1,3-dioxolan-2-yl)phenyl]acetamide (500 mg, 1.75 mmol, 1 equiv) in tetrahydrofuran (20 mL) was added n-butyllithium (2.5M in hexanes, 1.75 mL, 4.37 mmol, 2.5 equiv) under N₂. The resulting mixture was stirred at −78° C. for 1 h. Stirring was continued at −78° C. for 1 h under CO₂. The reaction mixture was concentrated in vacuo to provide crude 3-(1,3-dioxolan-2-yl)-4-acetamidobenzoic acid (Intermediate 45-1) (400 mg, 91%) as a light-yellow solid, which was used in subsequent transformations without purification. MS (ESI) calculated for C₁₂H₁₃NO₅: 251.08 m/z, found 252.05 [M+H]⁺.

### Example 46: (S)—N-(5-(2-(2-aminopyridin-3-yl)-5-methoxy-3H-imidazo[4,5-b]pyridin-3-yl)-2,3-dihydro-1H-inden-1-yl)-3-formyl-4-hydroxybenzamide

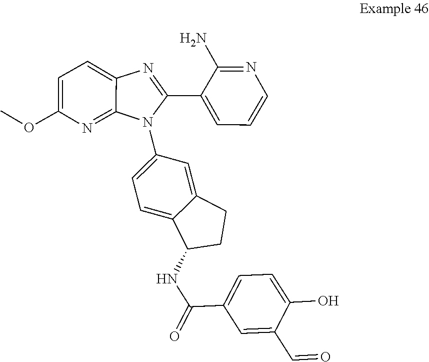 [SMILES: COc1ccc2nc(-c3cccnc3N)n(-c3ccc4c(c3)CC[C@@H]4NC(=O)c3ccc(O)c(C=O)c3)c2n1]

Example 46 was prepared in a manner analogous to Example 12 using Intermediate 46-1 in place of Intermediate 1-3 and Intermediate 5-1 in place of Intermediate 12-1. MS (ESI) calculated for C₂₉H₂₄N₆O₄: 520.19 m/z, found 521.05 [M+H]⁺. ¹H NMR (400 MHz, DMSO-d₆) δ 11.26 (s, 1H), 10.30 (s, 1H), 8.92 (d, J=8.3 Hz, 1H), 8.29 (d, J=2.4 Hz, 1H), 8.04-8.14 (m, 2H), 7.90-8.00 (m, 1H), 7.28-7.34 (m, 2H), 7.20-7.25 (m, 1H), 7.10-7.18 (m, 1H), 7.00-7.04 (m, 1H), 6.88 (s, 2H), 6.78-6.83 (m, 1H), 6.38-6.45 (m, 1H), 5.58-5.63 (m, 1H), 3.79 (s, 3H), 2.97-3.03 (m, 1H), 2.82-2.91 (m, 1H), 2.41-2.48 (m, 1H), 2.02-2.18 (m, 1H).

### Intermediate 46-1: (S)-3-(3-(1-amino-2,3-dihydro-1H-inden-5-yl)-5-methoxy-3H-imidazo[4,5-b]pyridin-2-yl)pyridin-2-amine

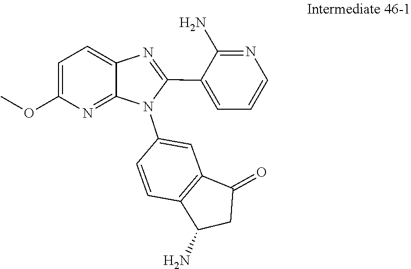 [SMILES: COc1ccc2nc(-c3cccnc3N)n(-c3ccc4c(c3)C(=O)C[C@@H]4N)c2n1]

Intermediate 46-1 was prepared in a manner analogous to Intermediate 13-2 using 6-methoxy-3-nitropyridin-2-amine in place of 6-methyl-3-nitropyridin-2-amine. MS (ESI) calculated for C₂₁H₂₀N₆O: 372.17 m/z, found 373.05 [M+H]⁺.

### Example 47: N-[(1S)-5-[2-(2-aminopyridin-3-yl)-5-phenylimidazo[4,5-b]pyridin-3-yl]-2,3-dihydro-1H-inden-1-yl]-4-cyclopropaneamido-3-formylbenzamide

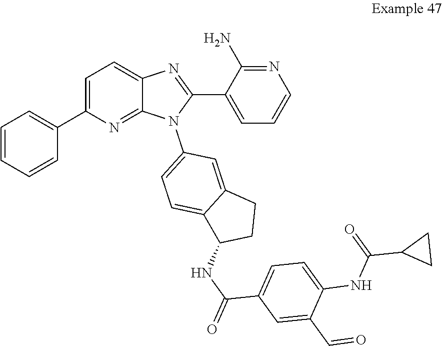 [SMILES: Nc1ncccc1-c1nc2ccc(-c3ccccc3)nc2n1-c1ccc2c(c1)CC[C@@H]2NC(=O)c1ccc(NC(=O)C2CC2)c(C=O)c1]

Example 47 was prepared in a manner analogous to Example 12 using Intermediate 47-1 in place of Intermediate 12-1. MS (ESI) calculated for C₃₈H₃₁N₇O₃: 633.25 m/z, found 634.25 [M+H]⁺. ¹H-NMR (400 MHz, DMSO-d₆) δ (ppm): 10.00 (s, 1H), 8.45-8.46 (m, 1H), 8.19-8.35 (m, 3H), 8.02-8.09 (m, 4H), 7.82-7.85 (m, 1H), 7.39-7.51 (m, 6H), 6.84-6.87 (m, 1H), 5.63-5.67 (m, 1H), 2.92-3.05 (m, 2H), 2.50 (s, 1H), 2.09-2.14 (m, 1H), 1.84-1.88 (m, 1H), 0.88-0.94 (m, 4H).

### Intermediate 47-1: 4-(cyclopropanecarboxamido)-3-(1,3-dioxolan-2-yl)benzoic acid

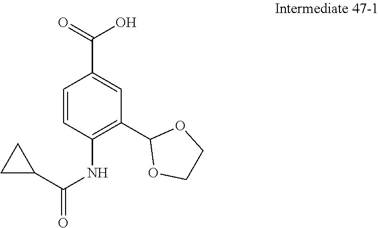 [SMILES: O=C(O)c1ccc(NC(=O)C2CC2)c(C2OCCO2)c1]

### Synthetic Route:

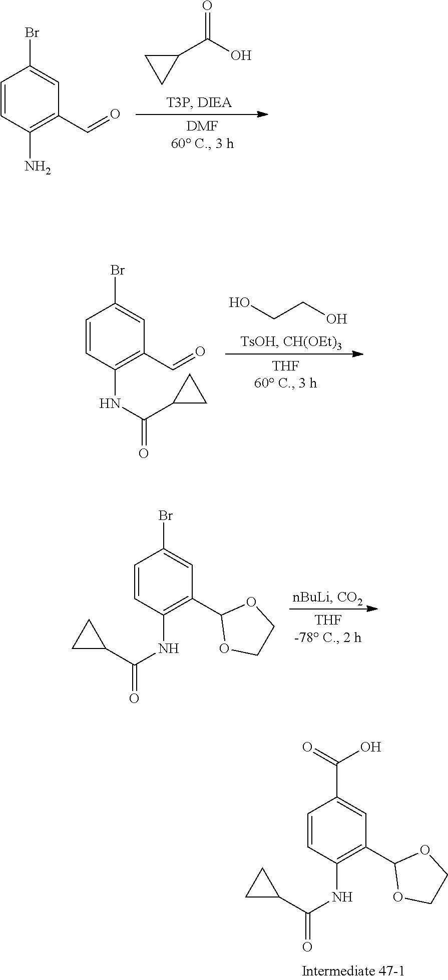

### Step 1: Synthesis of N-(4-bromo-2-formylphenyl)cyclopropanecarboxamide

To a solution of 2-amino-5-bromobenzaldehyde (1 g, 5.0 mmol, 1 equiv) in N,N-dimethylformamide (1 mL) was added N,N-diisopropylethylamine (1.94 g, 15 mmol, 3 equiv), propanephosphonic acid anhydride (6.36 g, 20 mmol, 4 equiv), cyclopropanecarboxylic acid (0.86 g, 9.10 mmol, 2 equiv). The resulting mixture was stirred at 60° C. for 3 h. The reaction mixture was concentrated in vacuo and purified by reverse-phase flash column chromatography on C18 silica gel using a 20-95% gradient of acetonitrile in water (+0.05% ammonium bicarbonate) with a 40 min hold at 70% acetonitrile to afford N-(4-bromo-2-formylphenyl)cyclopropanecarboxamide (530 mg, 40%) as a white solid. MS (ESI) calculated for C11H10BrNO2: 266.99 m/z, found 268.00 [M+H]⁺.

### Step 2: Synthesis of N-[4-bromo-2-(1,3-dioxolan-2-yl)phenyl]cyclopropanecarboxamide

To a solution of N-(4-bromo-2-formylphenyl)cyclopropanecarboxamide (530 mg, 1.98 mmol, 1 equiv) in toluene (30 mL) was added ethylene glycol (613 mg, 9.89 mmol, 5 equiv), p-toluenesulfonic acid (34 mg, 0.20 mmol, 0.1 equiv) and triethyl orthoformate (879 mg, 5.93 mmol, 3 equiv). The resulting mixture was stirred at 90° C. for 3 h. The reaction mixture was concentrated in vacuo and the resulting residue was purified by silica gel column chromatography using a 0-10% gradient of ethyl acetate in petroleum ether to afford N-[4-bromo-2-(1,3-dioxolan-2-yl)phenyl]cyclopropanecarboxamide (510 mg, 83%) as a white solid. MS (ESI) calculated for C₁₃H₁₄BrNO₃: 311.02 m/z, found 311.95 [M+H]⁺.

### Step 3: Synthesis of 4-cyclopropaneamido-3-(1,3-dioxolan-2-yl)benzoic acid (Intermediate 47-1)

To a solution of N-[4-bromo-2-(1,3-dioxolan-2-yl)phenyl]cyclopropanecarboxamide (250 mg, 0.80 mmol, 1 equiv) in tetrahydrofuran (10 mL) was added dropwise n-butyllithium solution (2.5 M in hexane, 0.80 mL, 2.00 mmol) at −78° C. under N₂ atmosphere. The reaction mixture was stirred at −78° C. for 30 mins. The mixture was then stirred for 2 hours under carbon dioxide. The reaction was quenched with saturated aqueous ammonium chloride and extracted with diethyl ether/ethyl acetate (2×15 mL). The combined organic extracts were washed with brine (10 mL), dried over anhydrous sodium sulfate, filtered and concentrated in vacuo, to afford 4-cyclopropaneamido-3-(1,3-dioxolan-2-yl)benzoic acid (Intermediate 47-1) (290 mg) as a white solid, which was used in subsequent transformations without further purification. MS (ESI) calculated for C₁₄H₁₅NO₅: 277.10 m/z, found 278.10 [M+H]⁺.

### Example 48: N-[(1S)-5-[2-(2-aminopyridin-3-yl)-5-fluoroimidazo[4,5-b]pyridin-3-yl]-2,3-dihydro-1H-inden-1-yl]-3-formyl-4-hydroxybenzamide

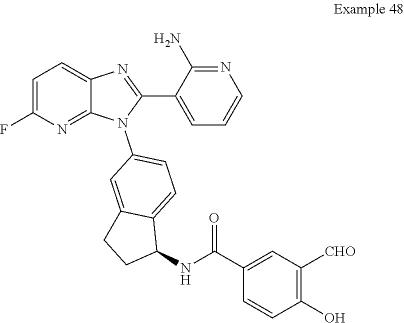 [SMILES: Nc1ncccc1-c1nc2ccc(F)nc2n1-c1ccc2c(c1)CC[C@@H]2NC(=O)c1ccc(O)c(C=O)c1]

Example 48 was prepared in a manner analogous to Example 12 using Intermediate 48-1 in place of Intermediate 1-3 and Intermediate 5-1 in place of Intermediate 12-1. MS (ESI) calculated for C₂₈H₂₁FN₆O₃: 508.17 m/z, found 509.20 [M+H]⁺. ¹H NMR (400 MHz, DMSO-d₆) δ (ppm): 10.28 (s, 1H), 8.87-8.88 (m, 1H), 8.34-8.38 (m, 1H), 8.26 (s, 1H), 7.98-8.04 (m, 2H), 7.36 (s, 1H), 7.27-7.36 (m, 2H), 7.15-7.20 (m, 1H), 6.99-7.13 (m, 1H), 6.97-6.98 (m, 1H), 6.43-6.44 (m, 1H), 5.58-5.61 (m, 1H), 2.99-3.03 (m, 1H), 2.87-2.89 (m, 1H), 2.50-2.51 (m, 1H) 2.04-2.09 (m, 1H). ¹⁹F NMR (376 MHz, DMSO-d₆) δ −73.81.

### Intermediate 48-1: (S)-3-(3-(1-amino-2,3-dihydro-1H-inden-5-yl)-5-fluoro-3H-imidazo[4,5-b]pyridin-2-yl)pyridin-2-amine

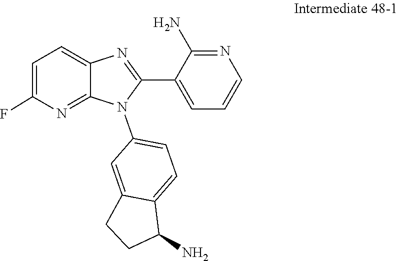 [SMILES: Nc1ncccc1-c1nc2ccc(F)nc2n1-c1ccc2c(c1)CC[C@@H]2N]

### Synthetic Route:

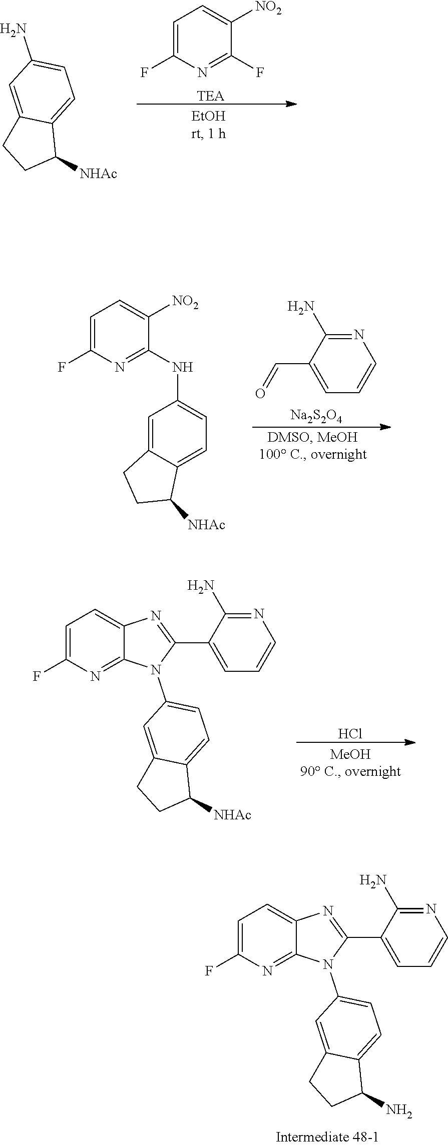

### Step 1: Synthesis of N-[(1S)-5-[(6-fluoro-3-nitropyridin-2-yl)amino]-2,3-dihydro-1H-inden-1-yl]acetamide

To a solution of (S)—N-(5-amino-2,3-dihydro-1H-inden-1-yl)acetamide (1.0 g, 5.3 mmol, 1 equiv) in ethanol (15 mL) was added triethylamine (2.66 g, 26.3 mmol, 5.0 equiv) and 2,6-difluoro-3-nitropyridine (842 mg, 5.26 mmol, 1 equiv). The resulting mixture was stirred at room temperature for 1 h, quenched with water, and filtered to afford N-[(1S)-5-[(6-fluoro-3-nitropyridin-2-yl)amino]-2,3-dihydro-1H-inden-1-yl]acetamide (800 mg, 46%) as a yellow solid, which was used without purification in the next step. MS (ESI) calculated for C₁₆H₁₅FN₄O₃: 330.11 m/z, found 331.10 [M+H]⁺.

### Step 2: Synthesis of N-[(1S)-5-[2-(2-aminopyridin-3-yl)-5-fluoroimidazo[4,5-b]pyridin-3-yl]-2,3-dihydro-1H-inden-1-yl]acetamide

To a solution of N-[(1S)-5-[(6-fluoro-3-nitropyridin-2-yl)amino]-2,3-dihydro-1H-inden-1-yl]acetamide (800 mg, 2.42 mmol, 1 equiv) in dimethyl sulfoxide (12 mL) and methanol (2 mL) were added 2-aminopyridine-3-carbaldehyde (355 mg, 2.91 mmol, 1.2 equiv) and sodium dithionite (1.05 g, 6.06 mmol, 2.5 equiv). The resulting mixture was stirred at 100° C. overnight. The mixture was allowed to cool to room temperature. The precipitated solids were collected by filtration, washed with water and purified by reverse-phase flash column chromatography on C18 silica gel using a 10-50% gradient of acetonitrile in water (+10 mmol/L ammonium bicarbonate) to afford N-[(1S)-5-[2-(2-aminopyridin-3-yl)-5-fluoroimidazo[4,5-b]pyridin-3-yl]-2,3-dihydro-1H-inden-1-yl]acetamide (200 mg, 19%) as a brown solid. MS (ESI) calculated for C₂₂H₁₉FN₆O: 402.16 m/z, found 403.10 [M+H]⁺.

### Step 3: Synthesis of 3-{3-[(1S)-1-amino-2,3-dihydro-1H-inden-5-yl]-5-fluoroimidazo[4,5-b]pyridin-2-yl}pyridin-2-amine (Intermediate 48-1)

To a solution of N-[(1S)-5-[2-(2-aminopyridin-3-yl)-5-fluoroimidazo[4,5-b]pyridin-3-yl]-2,3-dihydro-1H-inden-1-yl]acetamide (200 mg, 0.497 mmol, 1 equiv) in methanol (20 mL) was added hydrochloric acid (20 mL, conc.). The resulting mixture was stirred at 90° C. overnight. The mixture was concentrated in vacuo to afford 3-{3-[(1S)-1-amino-2,3-dihydro-1H-inden-5-yl]-5-fluoroimidazo[4,5-b]pyridin-2-yl}pyridin-2-amine (Intermediate 48-1) (180 mg, 82%) as a black solid, which was used directly in subsequent transformations without purification. MS (ESI) calculated for C₂₀H₁₇FN₆: 360.15 m/z, found 361.15 [M+H]⁺.

### Example 49: N-[(1S)-5-[2-(2-aminopyridin-3-yl)-5-cyclopropylimidazo[4,5-b]pyridin-3-yl]-2,3-dihydro-1H-inden-1-yl]-3-formyl-4-hydroxybenzamide

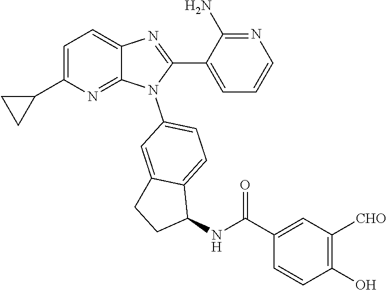 [SMILES: Nc1ncccc1-c1nc2ccc(C3CC3)nc2n1-c1ccc2c(c1)CC[C@@H]2NC(=O)c1ccc(O)c(C=O)c1]

Example 49 was prepared in a manner analogous to Example 19 using cyclopropylboronic acid in place of 3-(4-methylpiperazin-1-yl)phenylboronic acid. MS (ESI) calculated for C₃₁H₂₇N₆O₃: 530.20 m/z, found 531.25 [M+H]⁺. ¹H NMR (300 MHz, DMSO-d₆) δ (ppm): 10.28 (s, 1H), 8.30-8.31 (m, 1H), 8.04-8.12 (m, 3H), 7.68-7.71 (m, 1H), 7.23-7.36 (m, 4H), 7.07-7.10 (m, 1H), 6.77-6.81 (m, 1H), 5.58-5.64 (m, 1H), 2.85-3.56 (m, 2H), 2.51-2.52 (m, 1H), 2.06-2.22 (m, 2H), 0.95-0.98 (m, 2H), 0.83-0.84 (m, 2H).

### Example 50: N-[(1S)-5-[2-(2-aminopyridin-3-yl)-5-ethynylimidazo[4,5-b]pyridin-3-yl]-2,3-dihydro-1H-inden-1-yl]-3-formyl-4-hydroxybenzamide

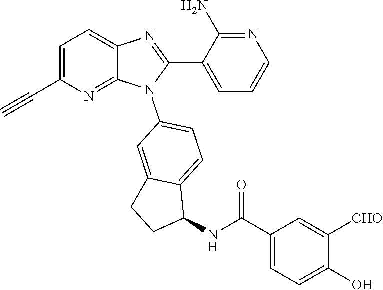 [SMILES: C#Cc1ccc2nc(-c3cccnc3N)n(-c3ccc4c(c3)CC[C@@H]4NC(=O)c3ccc(O)c(C=O)c3)c2n1]

### Synthetic Route:

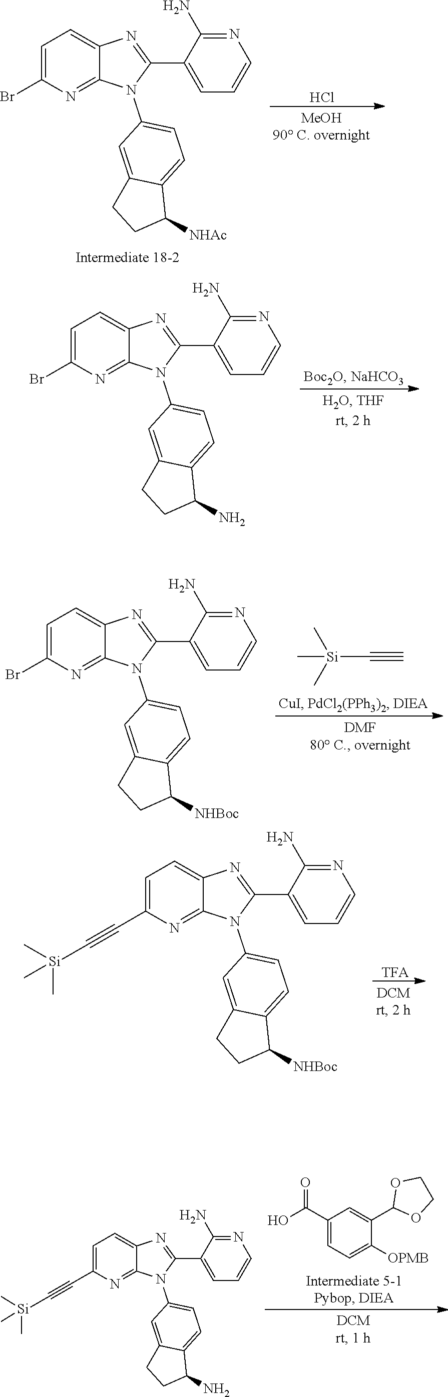

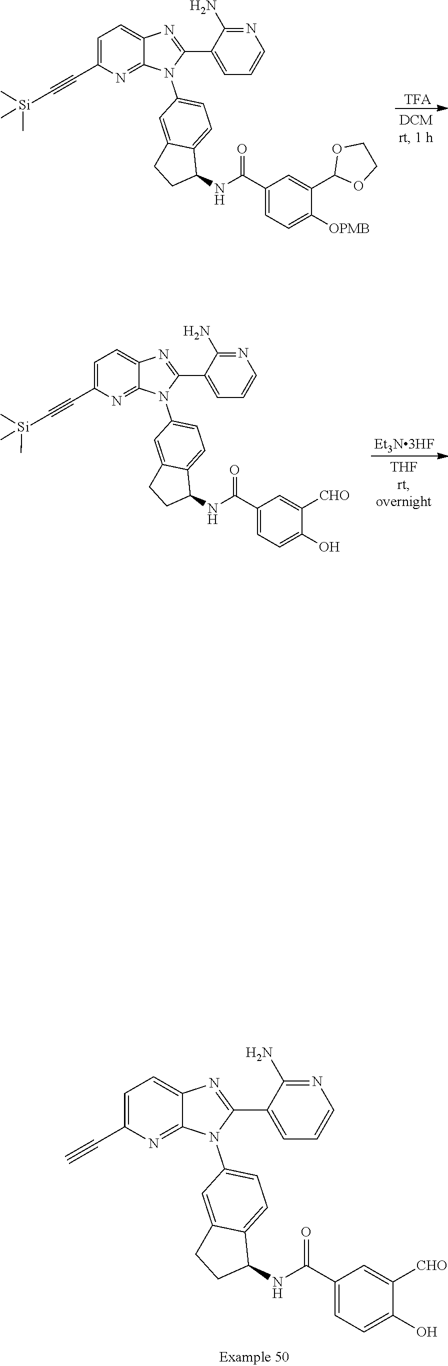

### Step 1: Synthesis of 3-{3-[(1S)-1-amino-2,3-dihydro-1H-inden-5-yl]-5-bromoimidazo[4,5-b] pyridin-2-yl}pyridin-2-amine

N-[(1S)-5-[2-(2-aminopyridin-3-yl)-5-bromoimidazo[4,5-b]pyridin-3-yl]-2,3-dihydro-1H-inden-1-yl]acetamide (Intermediate 18-2) (150 mg, 0.32 mmol, 1 equiv) was dissolved in methanol (2 mL) and hydrochloric acid (2 mL, concentrated). The resulting mixture was stirred at 90° C. overnight. The mixture was then cooled to room temperature, diluted with dichloromethane and concentrated in vacuo to provide 3-{3-[(1S)-1-amino-2,3-dihydro-1H-inden-5-yl]-5-bromoimidazo[4,5-b]pyridin-2-yl}pyridin-2-amine (150 mg, crude quantitative), which was used in subsequent transformations without purification. MS (ESI) calculated for C₂₀H₁₇BrN₆: 420.07 m/z, found 421.15 [M+H]⁺.

### Step 2: Synthesis of tert-butyl N-[(1S)-5-[2-(2-aminopyridin-3-yl)-5-bromoimidazo[4,5-b] pyridin-3-yl]-2,3-dihydro-1H-inden-1-yl]carbamate

3-{3-[(1S)-1-amino-2,3-dihydro-1H-inden-5-yl]-5-bromoimidazo[4,5-b]pyridin-2-yl} pyridin-2-amine (180 mg, 0.43 mmol, 1 equiv) was dissolved in tetrahydrofuran (20 mL) and saturated aqueous sodium bicarbonate (2 mL). di-tert-Butyl dicarbonate (186 mg, 0.854 mmol, 2 equiv) was added and the mixture was stirred for 2 h at room temperature. The reaction was quenched with water (50 mL). The resulting mixture was extracted with ethyl acetate (50 mL×3). The combined organic layers were washed with brine, dried over anhydrous sodium sulfate, filtered, and concentrated under reduced pressure. The residue was purified by trituration with petroleum ether to afford tert-butyl N-[(1S)-5-[2-(2-aminopyridin-3-yl)-5-bromoimidazo[4,5-b]pyridin-3-yl]-2,3-dihydro-1H-inden-1-yl]carbamate (200 mg, 81%) as a yellow oil. MS (ESI) calculated for C₂₅H₂₅BrN₆O₂: 520.12 m/z, found 521.15 [M+H]⁺.

### Step 3: Synthesis of tert-butyl N-[(1S)-5-[2-(2-aminopyridin-3-yl)-5-[2-(trimethylsilyl)ethynyl]imidazo[4,5-b]pyridin-3-yl]-2,3-dihydro-1H-inden-1-yl]carbamate

tert-Butyl N-[(1S)-5-[2-(2-aminopyridin-3-yl)-5-bromoimidazo[4,5-b]pyridin-3-yl]-2,3-dihydro-1H-inden-1-yl]carbamate (200 mg, 0.38 mmol, 1 equiv) and trimethylsilylacetylene (45 mg, 0.46 mmol, 1.2 equiv) were dissolved in N,N-dimethylformamide (5 mL). bis(Triphenylphosphine)palladium(II) dichloride (13 mg, 0.019 mmol, 0.05 equiv), N,N-diisopropylethylamine (74 mg, 0.58 mmol, 1.5 equiv) and copper (I) iodide (3.6 mg, 0.019 mmol, 0.05 equiv) were added and the mixture was stirred overnight at 80° C. The reaction was quenched with water (50 mL) and extracted with dichloromethane (20 mL×3). The combined organic layers were washed with brine, dried over anhydrous sodium sulfate, filtered, and concentrated in vacuo. The residue was purified by reverse-phase flash column chromatography on C18 silica gel using a 70-80% gradient of acetonitrile in water (+0.05 mmol/L 2,2,2-trifluoroacetic acid) to afford tert-butyl N-[(1S)-5-[2-(2-aminopyridin-3-yl)-5-[2-(trimethylsilyl)ethynyl]imidazo[4,5-b]pyridin-3-yl]-2,3-dihydro-1H-inden-1-yl]carbamate (150 mg, 58%) as a brown oil. MS (ESI) calculated for C₃₀H₃₄N₆O₂Si: 538.25 m/z, found 539.10 [M+H]⁺.

### Step 4: Synthesis of 3-{3-[(1S)-1-amino-2,3-dihydro-1H-inden-5-yl]-5-[2-(trimethylsilyl) ethynyl]imidazo[4,5-b]pyridin-2-yl}pyridin-2-amine

To a solution of tert-butyl N-[(1S)-5-[2-(2-aminopyridin-3-yl)-5-[2-(trimethylsilyl)ethynyl]imidazo[4,5-b]pyridin-3-yl]-2,3-dihydro-1H-inden-1-yl]carbamate (100 mg, 0.19 mmol, 1 equiv) in dichloromethane (3 mL) was added 2,2,2-trifluoroacetic acid (1 mL). The resulting mixture was stirred for 2 h at room temperature and concentrated in vacuo to afford 3-{3-[(1S)-1-amino-2,3-dihydro-1H-inden-5-yl]-5-[2-(trimethylsilyl) ethynyl]imidazo[4,5-b]pyridin-2-yl}pyridin-2-amine (110 mg, crude quantitative) as a brown oil, which was used in subsequent transformations without purification. MS (ESI) calculated for C₂₅H₂₆N₆Si: 438.20 m/z, found 439.20 [M+H]⁺.

### Step 5: Synthesis of N-[(1S)-5-[2-(2-aminopyridin-3-yl)-5-[2-(trimethylsilyl) ethynyl] imidazo[4,5-b]pyridin-3-yl]-2,3-dihydro-1H-inden-1-yl]-3-(1,3-dioxolan-2-yl)-4-[(4-methoxyphenyl)methoxy]benzamide

3-{3-[(1S)-1-amino-2,3-dihydro-1H-inden-5-yl]-5-[2-(trimethylsilyl) ethynyl]imidazo[4,5-b]pyridin-2-yl}pyridin-2-amine (80 mg, 0.18 mmol, 1 equiv) and N,N-diisopropylethylamine (70 mg, 0.55 mmol, 3 equiv) were dissolved in N,N-dimethylformamide (2.5 mL) and the resulting mixture was stirred for 20 minutes at room temperature. 3-(1,3-dioxolan-2-yl)-4-[(4-methoxyphenyl)methoxy]benzoic acid (Intermediate 5-1) (72 mg, 0.22 mmol, 1.2 equiv) and PyBOP (285 mg, 0.546 mmol, 3 equiv) were added to the mixture and stirring was continued for 1 h at room temperature. The reaction mixture was purified by reverse-phase flash column chromatography on C18 silica gel using a 70-80% gradient of acetonitrile in water (+0.05 mmol/L ammonium bicarbonate) to afford N-[(1S)-5-[2-(2-aminopyridin-3-yl)-5-[2-(trimethylsilyl) ethynyl]imidazo[4,5-b]pyridin-3-yl]-2,3-dihydro-1H-inden-1-yl]-3-(1,3-dioxolan-2-yl)-4-[(4-methoxyphenyl)methoxy]benzamide (60 mg, 31%) as a yellow semi-solid. MS (ESI) calculated for C₄₃H₄₂N₆O₅Si: 750.30 m/z, found 751.20 [M+H]⁺.

### Step 6: Synthesis of N-[(1S)-5-[2-(2-aminopyridin-3-yl)-5-[2-(trimethylsilyl)ethynyl]imidazo[4,5-b]pyridin-3-yl]-2,3-dihydro-1H-inden-1-yl]-3-formyl-4-hydroxybenzamide

N-[(1S)-5-[2-(2-aminopyridin-3-yl)-5-[2-(trimethylsilyl) ethynyl]imidazo[4,5-b] pyridin-3-yl]-2,3-dihydro-1H-inden-1-yl]-3-(1,3-dioxolan-2-yl)-4-[(4-methoxyphenyl)methoxy]benzamide (70 mg, 0.093 mmol, 1 equiv) was dissolved in 2,2,2-trifluoroacetic acid (1 mL) and the mixture was stirred for 1 h at room temperature. The resulting mixture was concentrated in vacuo and taken up into ethyl acetate. The pH of the mixture was brough to 8 with saturated aqueous sodium bicarbonate and the mixture was extracted 3 times with ethyl acetate. The combined organic layers were washed with brine, dried over anhydrous sodium sulfate, filtered, and concentrated in vacuo to afford crude N-[(1S)-5-[2-(2-aminopyridin-3-yl)-5-[2-(trimethylsilyl)ethynyl]imidazo[4,5-b]pyridin-3-yl]-2,3-dihydro-1H-inden-1-yl]-3-formyl-4-hydroxybenzamide (40 mg, 73%), which was used directly in the next step without further purification. MS (ESI) calculated for C₃₃H₃₀N₆O₃Si: 586.21 m/z, found 587.25 [M+H]⁺.

### Step 7: Synthesis of N-[(1S)-5-[2-(2-aminopyridin-3-yl)-5-ethynylimidazo[4,5-b]pyridin-3-yl]-2,3-dihydro-1H-inden-1-yl]-3-formyl-4-hydroxybenzamide (Example 50)

N-[(1S)-5-[2-(2-aminopyridin-3-yl)-5-[2-(trimethylsilyl)ethynyl]imidazo[4,5-b]pyridin-3-yl]-2,3-dihydro-1H-inden-1-yl]-3-formyl-4-hydroxybenzamide (40 mg, 0.068 mmol, 1 equiv) was dissolved in tetrahydrofuran (3 mL). Triethylamine trihydrofluoride (33 mg, 0.20 mmol, 3 equiv) was added and the resulting mixture was stirred overnight at room temperature. The reaction was quenched with saturated aqueous ammonium bicarbonate (50 mL). The resulting mixture was extracted with dichloromethane (20 mL×3). The combined organic layers were washed with brine, dried over anhydrous sodium sulfate, filtered, and concentrated in vacuo. The resulting residue was purified by preparative HPLC on a XSelect CSH Fluoro Phenyl column using a 19-44% gradient of acetonitrile in water (+0.05% 2,2,2-trifluoroacetic acid) to afford N-[(1S)-5-[2-(2-aminopyridin-3-yl)-5-ethynylimidazo[4,5-b]pyridin-3-yl]-2,3-dihydro-1H-inden-1-yl]-3-formyl-4-hydroxybenzamide (Example 50) (5.3 mg, 12%) as an off-white solid. MS (ESI) calculated for C₃₀H₂₂N₆O₃: 514.18 m/z, found 515.15 [M+H]⁺. ¹H NMR (300 MHz, DMSO-d₆) δ (ppm): 10.30 (s, 1H), 8.23-8.30 (m, 2H), 8.04-8.11 (m, 2H), 7.71-7.69 (m, 1H), 7.59-7.62 (m, 1H), 7.35-7.41 (m, 2H), 7.25-7.28 (m, 1H), 7.06-7.09 (m, 1H), 6.71-6.75 (m, 1H), 5.59-5.64 (m, 1H), 4.29 (s, 1H), 2.93-3.04 (m, 3H), 2.04-2.08 (m, 1H).

### Example 51: N-[(1S)-5-[2-(2-aminopyridin-3-yl)-5-phenylimidazo[4,5-b]pyridin-3-yl]-2,3-dihydro-1H-inden-1-yl]-3-formyl-4-methanesulfonamidobenzamide

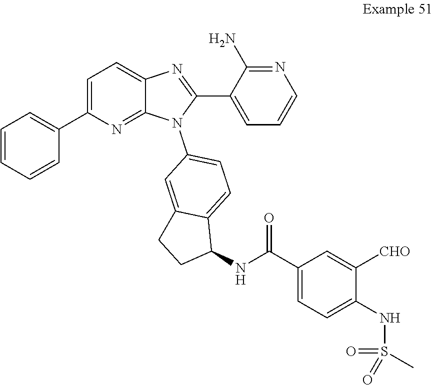 [SMILES: CS(=O)(=O)Nc1ccc(C(=O)N[C@H]2CCc3cc(-n4c(-c5cccnc5N)nc5ccc(-c6ccccc6)nc54)ccc32)cc1C=O]

Example 51 was prepared in a manner analogous so Example 12 using Intermediate 51-1 in place of Intermediate 12-1. MS (ESI) calculated for C₃₅H₂₉N₇O₄S: 643.72 m/z, found 644.15 [M+H]⁺. ¹H NMR (400 MHz, DMSO-d₆) δ (ppm): 10.08 (s, 1H), 9.10-9.12 (m, 1H), 8.49 (s, 1H), 8.23-8.27 (m, 2H), 7.96-8.04 (m, 4H), 7.60-7.63 (m, 1H), 7.41-7.49 (m, 2H), 7.37-7.39 (m, 3H), 7.25-7.34 (m, 2H), 6.44-6.47 (m, 1H), 5.66-5.68 (m, 1H), 3.23 (s, 3H), 3.01-3.03 (m, 1H), 2.89-2.93 (m, 1H), 2.55-2.56 (m, 1H), 2.07-2.13 (m, 1H).

### Intermediate 51-1: 3-(1,3-dioxolan-2-yl)-4-methanesulfonamidobenzoic acid

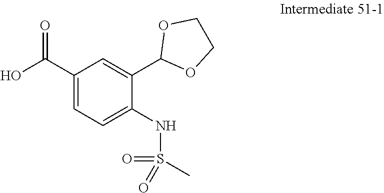 [SMILES: CS(=O)(=O)Nc1ccc(C(=O)O)cc1C1OCCO1]

### Synthetic Route:

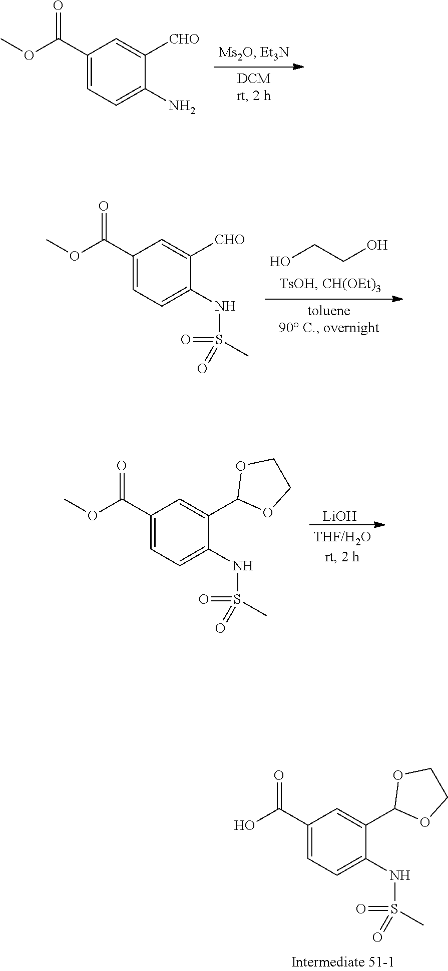

### Step 1: Synthesis of methyl 3-formyl-4-methanesulfonamidobenzoate

To a cooled (0° C.) solution of methyl 4-amino-3-formylbenzoate (1 g, 5.6 mmol, 1 equiv) in dichloromethane (20 mL) was added triethylamine (3.39 g, 33.5 mmol, 6 equiv) and methanesulfonic anhydride (3.89 g, 22.3 mmol, 4 equiv). The resulting solution was stirred for 1 h at room temperature and concentrated in vacuo. The crude residue was purified by silica gel column chromatography using a 0-25% gradient ethyl acetate in petroleum ether to provide methyl 3-formyl-4-methanesulfonamidobenzoate (990 mg, 69%) as a light-yellow oil. MS (ESI) calculated for C₁₀H₁₁NO₅S: 257.04 m/z, found 258.05 [M+H]⁺.

### Step 2: Synthesis of N-[4-bromo-2-(1,3-dioxolan-2-yl)phenyl]methanesulfonamide

To a solution of methyl 3-formyl-4-methanesulfonamidobenzoate (0.9 g, 3.50 mmol, 1 equiv) in toluene (10 mL) was added ethylene glycol (1.09 g, 17.5 mmol, 5 equiv), triethyl orthoformate (1.56 g, 10.5 mmol, 3 equiv) and p-toluenesulfonic acid (30 mg, 0.175 mmol, 0.05 equiv). The resulting solution was stirred at room temperature for 10 min and overnight at 90° C. The mixture was cooled to 0° C. and quenched with water (5 mL). The mixture was extracted with ethyl acetate (30 mL×3). The combined organic layers were washed with water (30 mL×3), dried over anhydrous sodium sulfate, filtered, and concentrated in vacuo. The residue was purified by silica gel column chromatography using a (0-50% gradient of ethyl acetate in petroleum ether) to provide methyl 3-(1,3-dioxolan-2-yl)-4-(methylsulfonamido)benzoate (0.56 g, 54%) as a light-yellow solid. MS (ESI) calculated for C₁₂H₁₅NO₆S: 301.06 m/z, found 323.10 [M+Na]⁺.

### Step 3: Synthesis of 3-(1,3-dioxolan-2-yl)-4-methanesulfonamidobenzoic acid (Intermediate 51-1)

To a cooled (0° C.) solution of methyl 3-(1,3-dioxolan-2-yl)-4-methanesulfonamidobenzoate (0.5 g, 1.66 mmol, 1 equiv) in tetrahydrofuran (20 mL) was added a solution of lithium hydroxide (0.12 g, 4.98 mmol, 3 equiv) in water (5 mL). The resulting solution was stirred for 2 h at room temperature and then partially concentrated in vacuo. 2N hydrochloric acid was added until the pH was 6. The resulting precipitate was filtered to provide 3-(1,3-dioxolan-2-yl)-4-methanesulfonamidobenzoic acid (Intermediate 51-1) (0.4 g, 84%) as a white solid. MS (ESI) calculated for C₁₁H₁₃NO₆S: 287.29 m/z, found 288.10 [M+H]⁺.

### Example 52: N-[(1S)-5-[2-(2-aminopyridin-3-yl)-5-phenylimidazo[4,5-b]pyridin-3-yl]-2,3-dihydro-1H-inden-1-yl]-4-(2,2-difluoroacetamido)-3-formylbenzamide

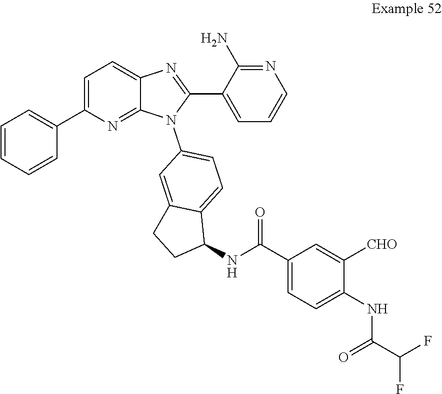 [SMILES: Nc1ncccc1-c1nc2ccc(-c3ccccc3)nc2n1-c1ccc2c(c1)CC[C@@H]2NC(=O)c1ccc(NC(=O)C(F)F)c(C=O)c1]

Example 52 was prepared in a manner analogous to Example 12 using Intermediate 52-1 in place of Intermediate 12-1. MS (ESI) calculated for C₃₆H₂₇F₂N₇O₃: 643.21 m/z, found 644.10 [M+H]⁺. ¹H NMR (400 MHz, DMSO-d₆) δ (ppm): 10.02-10.03 (m, 1H), 8.50-5.52 (m, 1H), 8.38-8.40 (m, 1H), 8.25-8.32 (m, 2H), 7.99-8.04 (m, 4H), 7.72-7.73 (m, 1H), 7.35-7.50 (m, 6H), 6.76-6.77 (m, 1H), 6.40-6.53 (m, 1H), 5.64 (s, 1H), 2.84-3.10 (m, 2H), 2.39 (s, 1H), 2.05-2.15 (m, 1H). ¹⁹F-NMR (400 MHz, DMSO-d₆) δ (ppm): −127.15.

### Intermediate 52-2: 4-(2,2-difluoroacetamido)-3-(1,3-dioxolan-2-yl)benzoic acid

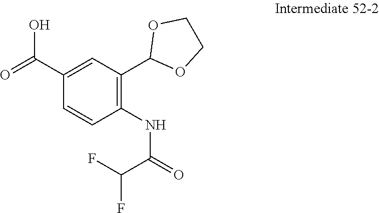 [SMILES: O=C(O)c1ccc(NC(=O)C(F)F)c(C2OCCO2)c1]

Intermediate 52-2 was prepared in a manner analogous to Intermediate 45-1 using 2,2-difluoroacetyl 2,2-difluoroacetate in place of acetic anhydride. MS (ESI) calculated for C₁₂H₁₁F₂NO₅: 287.06 m/z, found 288.00 [M+H]⁺.

### Example 53: N-[(1S)-5-[2-(2-aminopyridin-3-yl)-5-(morpholin-4-yl)imidazo[4,5-b]pyridin-3-yl]-2,3-dihydro-1H-inden-1-yl]-4-acetamido-3-formylbenzamide

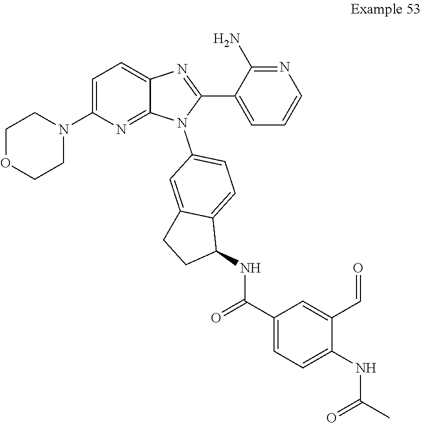 [SMILES: CC(=O)Nc1ccc(C(=O)N[C@H]2CCc3cc(-n4c(-c5cccnc5N)nc5ccc(N6CCOCC6)nc54)ccc32)cc1C=O]

Example 53 was prepared in a manner analogous to Example 12 using Intermediate 42-1 in place of Intermediate 1-3 and Intermediate 45-1 in place of Intermediate 12-1. MS (ESI) calculated for C₃₄H₃₂N₈O₄: 616.25 m/z, found 617.15 [M+H]⁺. ¹H NMR (400 MHz, DMSO-d₆) δ (ppm): 10.01 (s, 1H), 8.41-8.49 (m, 1H), 8.15-8.25 (m, 2H), 7.98-8.09 (m, 2H), 7.55-7.59 (m, 1H), 7.31-7.38 (m, 2H), 7.20-7.29 (m, 1H), 6.93-6.99 (m, 1H), 6.71-6.80 (m, 1H), 5.59-5.69 (m, 1H), 3.65-3.75 (m, 4H), 3.35-3.45 (m, 4H), 2.79-3.09 (m, 2H), 2.45-2.59 (m, 1H), 2.15-2.19 (m, 3H), 2.01-2.18 (m, 1H).

### Example 54: N-[(1S)-5-[2-(2-aminopyridin-3-yl)-5-phenylimidazo[4,5-b]pyridin-3-yl]-2,3-dihydro-1H-inden-1-yl]-4-formyl-5-hydroxypyrazolo[1,5-a]pyridine-3-carboxamide

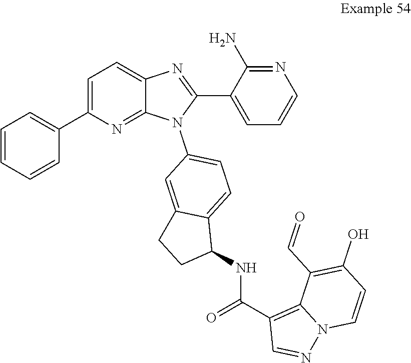 [SMILES: Nc1ncccc1-c1nc2ccc(-c3ccccc3)nc2n1-c1ccc2c(c1)CC[C@@H]2NC(=O)c1cnn2ccc(O)c(C=O)c12]

### Synthetic Route:

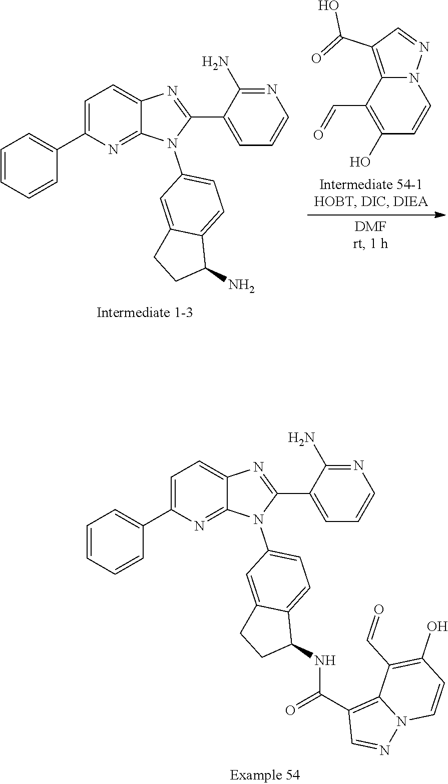

### Step 1: Synthesis of N-[(1S)-5-[2-(2-aminopyridin-3-yl)-5-phenylimidazo[4,5-b]pyridin-3-yl]-2,3-dihydro-1H-inden-1-yl]-4-formyl-5-hydroxypyrazolo[1,5-a]pyridine-3-carboxamide (Example 54)

To a solution of 4-formyl-5-hydroxypyrazolo[1,5-a]pyridine-3-carboxylic acid (Intermediate 54-1) (30 mg, 0.15 mmol, 1 equiv) and 3-{3-[(1S)-1-amino-2,3-dihydro-1H-inden-5-yl]-5-phenylimidazo[4,5-b]pyridin-2-yl}pyridin-2-amine (Intermediate 1-3) (73 mg, 0.18 mmol, 1.2 equiv) in N,N-dimethylformamide (1 mL) were added 1-hydroxybenzotriazole (22 mg, 0.16 mmol, 1.1 equiv), N,N-diisopropylethylamine (38 mg, 0.29 mmol, 2 equiv) and N,N′-diisopropylcarbodiimide (20 mg, 0.16 mmol, 1.1 equiv). The resulting mixture was stirred overnight at room temperature. The reaction mixture was purified by reverse-phase flash column chromatography on C18 silica gel using a 5-75% gradient of acetonitrile in water (+0.05% ammonium bicarbonate) to afford N-[(1S)-5-[2-(2-aminopyridin-3-yl)-5-phenylimidazo[4,5-b]pyridin-3-yl]-2,3-dihydro-1H-inden-1-yl]-4-formyl-5-hydroxypyrazolo[1,5-a]pyridine-3-carboxamide (Example 54) (5.2 mg, 5.7%) as a yellow solid. MS (ESI) calculated for C₃₅H₂₆N₈O₃: 606.21 m/z, found 607.25 [M+H]⁺. ¹H NMR (300 MHz, DMSO-d₆) δ (ppm): 13.38-13.40 (m, 1H), 10.35-10.39 (m, 1H), 8.38-8.41 (m, 1H), 8.28-8.31 (m, 1H), 8.14-8.16 (m, 1H), 8.02-8.04 (m, 4H), 7.29-7.55 (m, 10H), 6.49-6.51 (m, 1H), 6.23-6.26 (m, 1H), 5.35-5.38 (m, 1H), 3.13-3.15 (m, 1H), 3.02-3.04 (m, 1H), 2.89-2.94 (m, 1H), 2.28-2.32 (m, 1H).

### Intermediate 54-1: 4-formyl-5-hydroxypyrazolo[1,5-a]pyridine-3-carboxylic acid

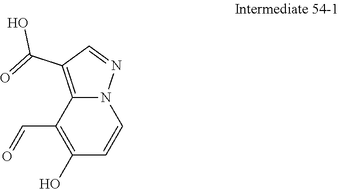 [SMILES: O=Cc1c(O)ccn2ncc(C(=O)O)c12]

### Synthetic Route:

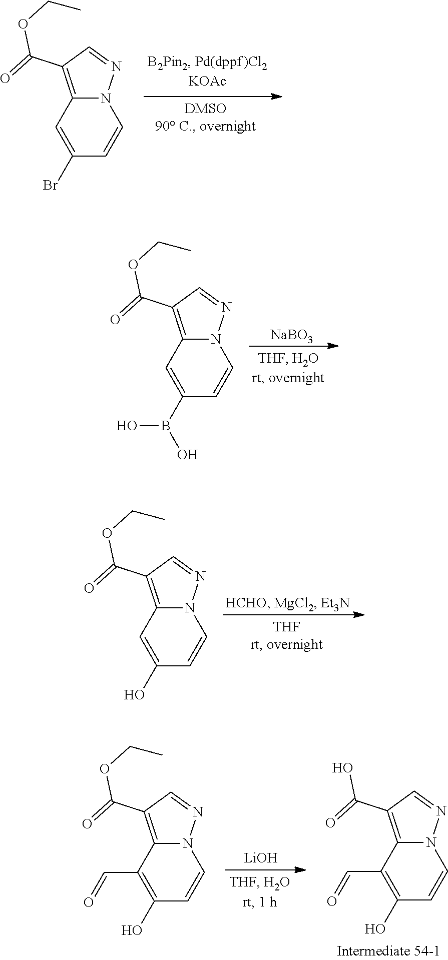

### Step 1: Synthesis of (3-(ethoxycarbonyl)pyrazolo[1,5-a]pyridin-5-yl)boronic acid

A solution of ethyl 5-bromopyrazolo[1,5-a]pyridine-2-carboxylate (3.00 g, 11.1 mmol, 1 equiv), bis(pinacolato)diboron (3.68 g, 14.5 mmol, 1.3 equiv), potassium acetate (1.64 g, 16.7 mmol, 1.5 equiv) and [1,1′-bis(diphenylphosphino)ferrocene]dichloropalladium(II) (0.82 g, 1.12 mmol, 0.1 equiv) in dimethyl sulfoxide (30 mL) was stirred for 2 h at 90° C. under nitrogen atmosphere. The reaction was quenched with water (50 mL) at room temperature. The resulting solution was extracted with ethyl acetate (20 mL×3). The organic layers were combined, washed with brine, dried over anhydrous sodium sulfate, filtered, and concentrated in vacuo. The resulting residue was purified by silica gel column chromatography using a 0-100% gradient of ethyl acetate in petroleum ether to provide 2-(ethoxycarbonyl)pyrazolo[1,5-a]pyridin-5-ylboronic acid (1.5 g, 57%) as a yellow solid. MS (ESI) calculated for C₁₀H₁₁BN₂O₄: 234.08 m/z, found 235.10 [M+H]⁺.

### Step 2: Synthesis of ethyl 5-hydroxypyrazolo[1,5-a]pyridine-3-carboxylate

To a solution of 2-(ethoxycarbonyl)pyrazolo[1,5-a]pyridin-5-ylboronic acid (1.50 g, 6.41 mmol, 1 equiv) in water (10 mL) and tetrahydrofuran (5 mL) was added sodium perborate (2.62 g, 32.1 mmol, 5 equiv). The resulting mixture was stirred overnight at room temperature. The reaction was quenched with water (50 mL) and extracted with ethyl acetate (20 mL×3). The combined organic layers were washed with brine, dried over anhydrous sodium sulfate, filtered, and concentrated in vacuo. The crude product was purified by reverse-phase flash column chromatography on C18 silica gel using a 5-45% gradient of acetonitrile in water (+0.05% ammonium bicarbonate) to afford ethyl 5-hydroxypyrazolo[1,5-a]pyridine-2-carboxylate (1.0 g, 76%) as a yellow solid. MS (ESI) calculated for C₁₀H₁₀N₂O₃: 206.07 m/z, found 207.15[M+H]⁺.

### Step 3: Synthesis of ethyl 4-formyl-5-hydroxypyrazolo[1,5-a]pyridine-3-carboxylate

To a solution of ethyl 5-hydroxypyrazolo[1,5-a]pyridine-3-carboxylate (1.00 g, 4.85 mmol, 1 equiv), polyoxymethylene (2.18 g, 24.3 mmol, 5 equiv) and magnesium (II) chloride (0.69 g, 7.3 mmol, 1.5 equiv) in tetrahydrofuran (10 mL) was added triethylamine (1.23 g, 12.1 mmol, 2.5 equiv). The resulting mixture was stirred for 2 h at room temperature. The reaction was quenched with water (50 mL) at room temperature. The resulting mixture was extracted with ethyl acetate (20 mL×3). The combined organic layers were washed with saturated aqueous ammonium chloride and brine, dried over anhydrous sodium sulfate, filtered, and concentrated in vacuo. The crude product was purified by reverse-phase flash column chromatography on C18 silica gel using a 20-65% gradient of acetonitrile in water (+0.05% ammonium bicarbonate) to afford ethyl 4-formyl-5-hydroxypyrazolo[1,5-a]pyridine-3-carboxylate (410 mg, 34%) as a yellow solid. MS (ESI) calculated for C₁₁H₁₀N₂O₄: 234.06 m/z, found 235.15 [M+H]⁺.

### Step 4: Synthesis of 4-formyl-5-hydroxypyrazolo[1,5-a]pyridine-3-carboxylic acid (Intermediate 54-1)

To a solution of ethyl 4-formyl-5-hydroxypyrazolo[1,5-a]pyridine-3-carboxylate (100 mg, 0.427 mmol, 1 equiv) in tetrahydrofuran (2 mL) was added a solution of lithium hydroxide (31 mg, 1.3 mmol, 3 equiv) in water (1 mL) and the reaction mixture was stirred at room temperature for 1 h. The pH of the solution was adjusted to 5-6 with 1M hydrochloric acid. The resulting mixture was extracted with ethyl acetate (10 mL×3), dried over anhydrous sodium sulfate, filtered, and concentrated in vacuo to afford 4-formyl-5-hydroxypyrazolo[1,5-a]pyridine-3-carboxylic acid (Intermediate 54-1) (30 mg, 34%) as a white solid, which was used in subsequent transformations without further purification. MS (ESI) calculated for C₉H₆N₂O₄: 206.03 m/z, found 205.11 [M−H]⁻.

### Example 55: (S)—N-(5-(2-(2-aminopyridin-3-yl)-5-(trifluoromethyl)-3H-imidazo[4,5-b]pyridin-3-yl)-2,3-dihydro-1H-inden-1-yl)-3-formyl-4-hydroxybenzamide

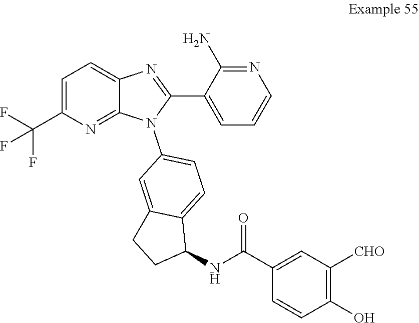 [SMILES: Nc1ncccc1-c1nc2ccc(C(F)(F)F)nc2n1-c1ccc2c(c1)CC[C@@H]2NC(=O)c1ccc(O)c(C=O)c1]

Example 55 was prepared in a manner analogous to Example 7 using Intermediate 55-1 in place of Intermediate 1-2. MS (ESI) calculated for C₂₉H₂₁F₃N₆O₃: 558.16 m/z, found 559.15[M+H]⁺. ¹H NMR (300 MHz, DMSO-d₆) δ (ppm) 10.22 (s, 1H), 8.32-8.47 (m, 1H), 8.14-8.28 (m, 1H), 7.85-8.04 (m, 3H), 7.21-7.39 (m, 4H), 6.92-7.01 (m, 1H), 6.49-6.59 (m, 1H), 5.55-5.65 (m, 1H), 2.83-3.10 (m, 2H), 2.51-2.54 (m, 1H), 1.99-2.16 (m, 1H). ¹⁹F NMR (282 MHz, DMSO-d₆) δ (ppm) −64.27.

### Intermediate 55-1: (S)-3-(3-(1-amino-2,3-dihydro-1H-inden-5-yl)-5-(trifluoromethyl)-3H-imidazo[4,5-b]pyridin-2-yl)pyridin-2-amine

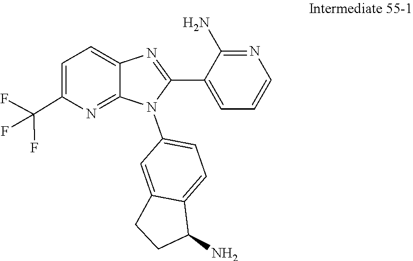 [SMILES: Nc1ncccc1-c1nc2ccc(C(F)(F)F)nc2n1-c1ccc2c(c1)CC[C@@H]2N]

Intermediate 55-1 was prepared in a manner analogous to Intermediate 13-2 using 2-chloro-3-nitro-6-(trifluoromethyl)pyridine in place of 6-methyl-3-nitropyridin-2-amine. MS (ESI) calculated for C₂₁H₁₇F₃N₆: 410.15 m/z, found 411.15 [M+H]⁺.

### Example 56: N-[(1S)-5-[2-(2-aminopyridin-3-yl)-5-(pyrazol-1-yl)imidazo[4,5-b]pyridin-3-yl]-2,3-dihydro-1H-inden-1-yl]-4-acetamido-3-formylbenzamide

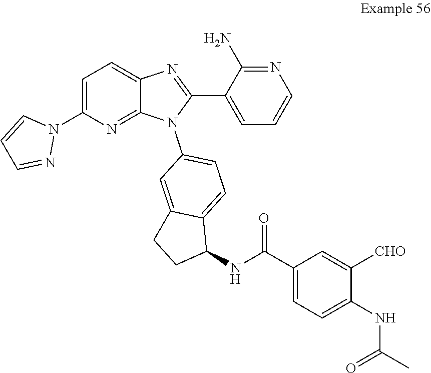 [SMILES: CC(=O)Nc1ccc(C(=O)N[C@H]2CCc3cc(-n4c(-c5cccnc5N)nc5ccc(-n6cccn6)nc54)ccc32)cc1C=O]

Example 56 was prepared in a manner analogous to Example 7 using Intermediate 33-1 in place of Intermediate 1-2 and Intermediate 45-1 in place of Intermediate 5-1. MS (ESI) calculated for C₃₃H₂₇N₉O₃: 597.22 m/z, found 598.20 [M+H]⁺. ¹H NMR (400 MHz, DMSO-d₆) δ (ppm): 9.99 (s, 1H), 8.27-8.43 (m, 3H), 8.15-8.24 (m, 2H), 7.96-8.03 (m, 2H), 7.57-7.81 (m, 1H), 7.39-7.40 (m, 3H), 7.26-7.31 (m, 1H), 6.55-6.56 (m, 2H), 5.62-5.66 (m, 1H), 2.91-3.09 (m, 2H), 2.41-2.54 (m, 5H).

### Example 57: N-[(1S)-5-[2-(2-aminopyridin-3-yl)-5-(1,3-thiazol-2-yl)imidazo[4,5-b]pyridin-3-yl]-2,3-dihydro-1H-inden-1-yl]-3-formyl-4-hydroxybenzamide

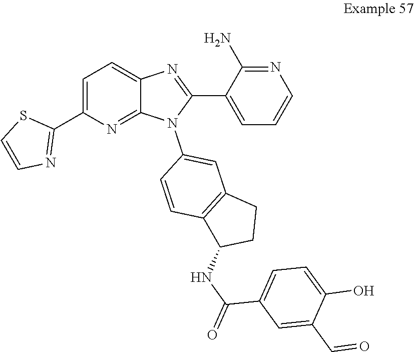 [SMILES: Nc1ncccc1-c1nc2ccc(-c3nccs3)nc2n1-c1ccc2c(c1)CC[C@@H]2NC(=O)c1ccc(O)c(C=O)c1]

Example 57 was prepared in a manner analogous to Example 12 using Intermediate 57-1 in place of Intermediate 1-3 and Intermediate 57-2 in place of Intermediate 12-1. MS (ESI) calculated for C₃₁H₂₃N₇O₃S: 573.16 m/z, found 574.15 [M+H]⁺. ¹H NMR (400 MHz, DMSO-d₆) δ (ppm): 10.28-10.30 (m, 1H), 8.37-8.39 (m, 1H), 8.29-8.30 (m, 1H), 8.10-8.25 (m, 1H), 8.06-8.08 (m, 2H), 7.95-7.96 (m, 1H), 7.78-7.79 (m, 2H), 7.46 (s, 1H), 7.32-7.37 (m, 2H), 7.07-7.09 (m, 1H), 6.79-6.83 (m, 1H), 5.60-5.64 (m, 1H), 3.02-3.06 (m, 1H), 2.86-2.94 (m, 1H), 2.50-2.58 (m, 1H), 2.06-2.16 (m, 1H).

### Intermediate 57-1: (S)-3-(3-(1-amino-2,3-dihydro-1H-inden-5-yl)-5-(thiazol-2-yl)-3H-imidazo[4,5-b]pyridin-2-yl)pyridin-2-amine

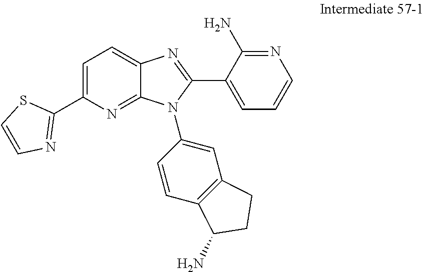 [SMILES: Nc1ncccc1-c1nc2ccc(-c3nccs3)nc2n1-c1ccc2c(c1)CC[C@@H]2N]

### Synthetic Route:

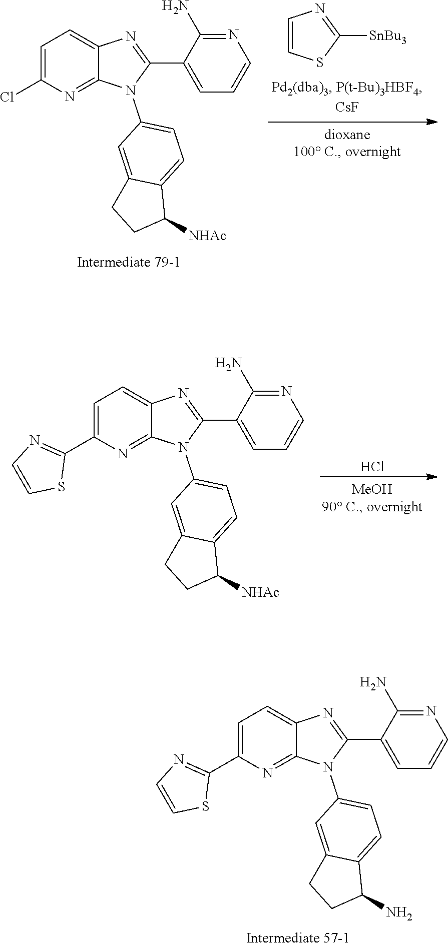

### Step 1: Synthesis of N-[(1S)-5-[2-(2-aminopyridin-3-yl)-5-(1,3-thiazol-2-yl)imidazo[4,5-b]pyridin-3-yl]-2,3-dihydro-1H-inden-1-yl]acetamide

To a solution of N-[(1S)-5-[2-(2-aminopyridin-3-yl)-5-chloroimidazo[4,5-b]pyridin-3-yl]-2,3-dihydro-1H-inden-1-yl]acetamide (Intermediate 79-1) (300 mg, 0.716 mmol, 1 equiv) and 2-(tributylstannyl)-1,3-thiazole (1.34 g, 3.58 mmol, 5 equiv) in 1,4-dioxane (5 mL) were added tris(dibenzylideneacetone)dipalladium(0) (67 mg, 0.072 mmol, 0.1 equiv), tri-tert-butylphosphonium tetrafluoroborate (21 mg, 0.072 mmol, 0.1 equiv) and cesium fluoride (272 mg, 1.79 mmol, 2.5 equiv). The resulting mixture was stirred under nitrogen atmosphere for 12 h at 100° C. The resulting mixture was concentrated under reduced pressure and residue was purified by reverse-phase flash column chromatography on C18 silica gel using a 20-95% gradient of acetonitrile in water (+0.05% ammonium bicarbonate) with a 5-minute hold at 70% acetonitrile to afford N-[(1S)-5-[2-(2-aminopyridin-3-yl)-5-(1,3-thiazol-2-yl)imidazo[4,5-b]pyridin-3-yl]-2,3-dihydro-1H-inden-1-yl]acetamide (250 mg, 67%) as a brown solid. MS (ESI) calculated for C₂₅H₂₁N₇OS: 467.15 m/z, found 468.15 [M+H]⁺.

### Step 2: Synthesis of 3-{3-[(1S)-1-amino-2,3-dihydro-1H-inden-5-yl]-5-(1,3-thiazol-2-yl)imidazo[4,5-b]pyridin-2-yl}pyridin-2-amine (Intermediate 57-1)

To a solution of N-[(1S)-5-[2-(2-aminopyridin-3-yl)-5-(1,3-thiazol-2-yl)imidazo[4,5-b]pyridin-3-yl]-2,3-dihydro-1H-inden-1-yl]acetamide (250 mg, 0.535 mmol, 1 equiv) in methanol (20 mL) was added hydrochloric acid (20 mL, conc.). The resulting mixture was stirred at 90° C. overnight. The solvent was removed by distillation under vacuum to afford crude 3-{3-[(1S)-1-amino-2,3-dihydro-1H-inden-5-yl]-5-(1,3-thiazol-2-yl)imidazo[4,5-b]pyridin-2-yl}pyridin-2-amine (Intermediate 57-1) as a black solid, which was used in subsequent transformations without purification. MS (ESI) calculated for C₂₃H₁₉N₇S: 425.14 m/z, found 426.10 [M+H]⁺.

### Intermediate 57-2: 4-(benzyloxy)-3-(1,3-dioxolan-2-yl)benzoic acid

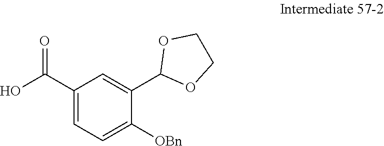 [SMILES: O=C(O)c1ccc(OCc2ccccc2)c(C2OCCO2)c1]

### Synthetic Route:

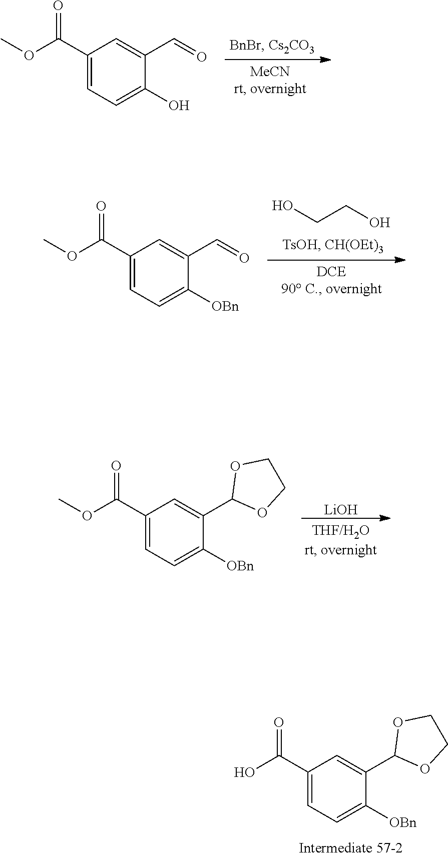

### Step 1: Synthesis of methyl 4-(benzyloxy)-3-formylbenzoate

To a stirred solution of methyl 3-formyl-4-hydroxybenzoate (100 g, 555 mmol, 1 equiv) and cesium carbonate (271 g, 833 mmol, 1.5 equiv) in acetonitrile (1 L) was added benzyl bromide (114 g, 666 mmol, 1.2 equiv) dropwise at room temperature. The resulting mixture was stirred overnight at room temperature. The resulting mixture was filtered, rinsing with ethyl acetate (3×200 mL). The filtrate was concentrated under reduced pressure and the resulting crude residue was recrystallized from petroleum ether (200 mL) to afford methyl 4-(benzyloxy)-3-formylbenzoate (90 g, 60%) as a white solid. MS (ESI) calculated for C₁₆H₁₄O₄: 270.20 m/z, found 271.2 [M+H]⁺.

### Step 2: Synthesis of methyl 4-(benzyloxy)-3-(1,3-dioxolan-2-yl)benzoate

To a stirred solution of methyl 4-(benzyloxy)-3-formylbenzoate (90.0 g, 333 mmol, 1 equiv) in 1,2-dichloroethane (1 L) was added ethylene glycol (103 g, 1.66 mol, 5 equiv), triethyl orthoformate (148 g, 999 mmol, 3 equiv) and p-toluenesulfonic acid (5.73 g, 33.3 mmol, 0.1 equiv) in portions at room temperature. The resulting mixture was stirred overnight at 90° C. The mixture was cooled to room temperature and quenched with water (500 mL). The resulting mixture was extracted with dichloromethane (3×200 mL). The combined organic layers were washed with brine (200 mL), dried over anhydrous sodium sulfate, filtered, and concentrated under reduced pressure. The residue was purified by silica gel column chromatography using a 0-50% gradient of ethyl acetate in petroleum ether to afford methyl 4-(benzyloxy)-3-(1,3-dioxolan-2-yl)benzoate (69 g, 66%) as a white solid. ME (ESI) calculated for C₁₈H₁₈O₅: 314.12 m/z, found 315.20 [M+H]⁺.

### Step 3: Synthesis of 4-(benzyloxy)-3-(1,3-dioxolan-2-yl)benzoic acid (Intermediate 57-2)

To a stirred solution of methyl 4-(benzyloxy)-3-(1,3-dioxolan-2-yl)benzoate (65.0 g, 207 mmol, 1 equiv) in tetrahydrofuran (500 mL) was added a solution of lithium hydroxide (9.90 g, 414 mmol, 2 equiv) in water (500 mL) portion wise at room temperature and stirring was continued overnight. The resulting mixture was partially concentrated in vacuo and acidified to pH 5-6 with 1M aqueous hydrochloric acid. The precipitated solids were collected by filtration and washed with water (100 mL). The resulting solid was dried to afford 4-(benzyloxy)-3-(1,3-dioxolan-2-yl)benzoic acid (Intermediate 57-2) (56 g, 90%) as a white solid. MS (ESI) calculated for C₁₇H₁₆O₅: 300.10 m/z, found 299.2 [M−H]⁻.

### Example 58: N-[(1S)-5-[2-(2-aminopyridin-3-yl)-5-(pyrimidin-5-yl)imidazo[4,5-b]pyridin-3-yl]-2,3-dihydro-1H-inden-1-yl]-3-formyl-4-hydroxybenzamide

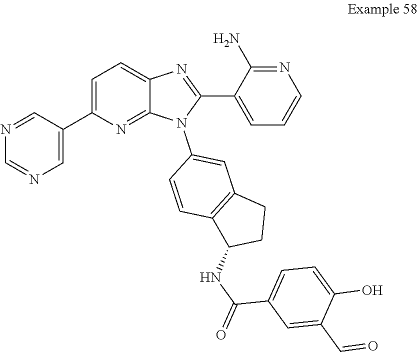 [SMILES: Nc1ncccc1-c1nc2ccc(-c3cncnc3)nc2n1-c1ccc2c(c1)CC[C@@H]2NC(=O)c1ccc(O)c(C=O)c1]

Example 58 was prepared in a manner analogous to Example 19 using pyrimidin-5-ylboronic acid in place of 3-(4-methylpiperazin-1-yl)phenylboronic acid and PyBOP in place of HATU. MS (ESI) calculated for C₃₂H₂₄N₈O₃: 568.20 m/z, found 569.30 [M+H]⁺. ¹H NMR (400 MHz, DMSO-d₆) δ (ppm): 10.21 (s, 1H), 9.31-9.39 (m, 2H), 9.15-9.21 (m, 1H), 8.30-8.39 (m, 1H), 8.20-8.29 (m, 1H), 8.09-8.15 (m, 1H), 7.99-8.05 (m, 1H), 7.88-7.99 (m, 1H), 7.35-7.45 (m, 2H), 7.25-7.34 (m, 2H), 6.75-6.85 (m, 1H), 6.45-6.55 (m, 1H), 5.58-5.68 (m, 1H), 3.01-3.15 (m, 1H), 2.85-2.99 (m, 1H), 2.45-2.60 (m, 1H), 2.01-2.18 (m, 1H).

### Example 59: (S)—N-(5-(2-(2-aminopyridin-3-yl)-5-(1-methyl-1H-pyrazol-3-yl)-3Himidazo[4,5-b]pyridin-3-yl)-2,3-dihydro-1H-inden-1-yl)-3-formyl-4-hydroxybenzamide

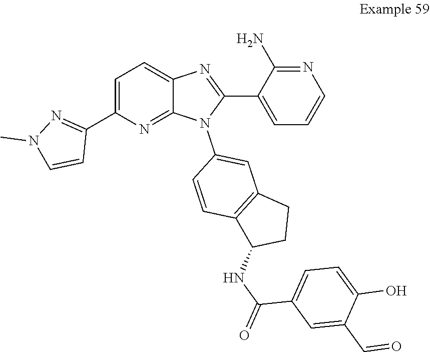 [SMILES: Cn1ccc(-c2ccc3nc(-c4cccnc4N)n(-c4ccc5c(c4)CC[C@@H]5NC(=O)c4ccc(O)c(C=O)c4)c3n2)n1]

Example 59 was prepared in a manner analogous to Example 19 using 1-methyl-3-(4,4,5,5-tetramethyl-1,3,2-dioxaborolan-2-yl)pyrazole in place of 3-(4-methylpiperazin-1-yl)phenylboronic acid, PyBOP in place of HATU and Intermediate 57-2 in place of Intermediate 5-1. MS (ESI) calculated for C₃₂H₂₆N₈O₃: 570.21 m/z, found 571.20 [M+H]⁺. ¹H NMR (400 MHz, DMSO-d₆) δ (ppm): 10.25 (s, 1H), 8.22-8.25 (m, 2H), 8.00-8.07 (m, 1H), 7.96-7.99 (m, 2H), 7.69-7.73 (s, 2H), 7.40-7.42 (m, 1H), 7.29-7.34 (m, 2H), 7.05-7.07 (m, 1H), 6.75-6.77 (m, 1H), 6.63-6.65 (m, 1H), 5.57-5.59 (m, 1H), 3.90-3.92 (m, 3H), 3.00-3.06 (m, 1H), 2.85-2.90 (m, 1H), 2.39-2.41 (m, 1H), 2.03-2.09 (m, 1H).

### Example 60: (S)—N-(5-(2-(2-aminopyridin-3-yl)-5-(6-methylpyrazin-2-yl)-3H-imidazo[4,5-b]pyridin-3-yl)-2,3-dihydro-1H-inden-1-yl)-3-formyl-4-hydroxybenzamide

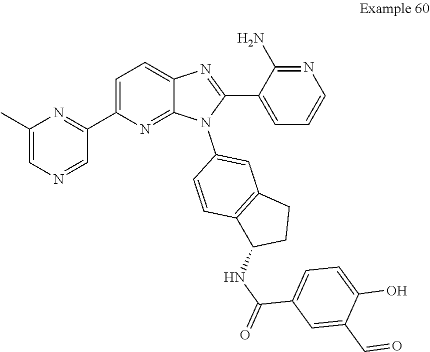 [SMILES: Cc1cncc(-c2ccc3nc(-c4cccnc4N)n(-c4ccc5c(c4)CC[C@@H]5NC(=O)c4ccc(O)c(C=O)c4)c3n2)n1]

Example 60 was prepared in a manner analogous to Example 19 using Intermediate 60-1 in place of 3-(4-methylpiperazin-1-yl)phenylboronic acid, PyBOP in place of HATU and Intermediate 57-2 in place of Intermediate 5-1. MS (ESI) calculated for C₃₃H₂₆N₈O₃: 582.21 m/z, found 583.15 [M+H]⁺. ¹H NMR (400 MHz, DMSO-d₆) δ (ppm): 10.31 (s, 1H), 9.15-9.17 (m, 1H), 8.93-8.94 (m, 1H), 8.41-8.56 (m, 1H), 8.30-8.41 (m, 2H), 8.03-8.09 (m, 2H), 7.39-7.44 (m, 1H), 7.31-7.44 (m, 3H), 7.00-7.03 (m, 2H), 6.45-6.48 (m, 1H), 5.65-5.67 (m, 1H), 2.90-3.04 (m, 2H), 2.50-2.67 (m, 3H), 2.29-2.32 (m, 1H), 2.07-2.11 (m, 1H).

### Intermediate 60-1: (6-methylpyrazin-2-yl)boronic acid

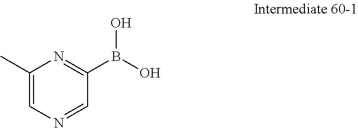

### Synthetic Route:

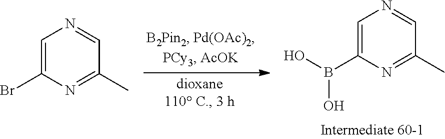

### Step 1: Synthesis of (6-methylpyrazin-2-yl)boronic acid (Intermediate 60-1)

A mixture of 2-bromo-6-methylpyrazine (1.00 g, 5.81 mmol, 1 equiv), bis(pinacolato)diboron (1.75 g, 6.89 mmol, 1.2 equiv), palladium(II) acetate (78.3 mg, 0.349 mmol, 0.06 equiv), potassium acetate (1.43 g, 14.6 mmol 2.5 equiv) and tricyclohexylphosphine (0.20 g, 0.71 mmol, 0.12 equiv) in 1,4-dioxane (10 mL) was stirred at 100° C. under nitrogen atmosphere for 2 h. The resulting mixture was concentrated in vacuo and the residue obtained was purified by reverse-phase column chromatography on C18 silica gel using a 0-4% gradient of acetonitrile in water (+0.05% ammonium bicarbonate) to afford (6-methylpyrazin-2-yl)boronic acid (Intermediate 60-1) as a brown solid (121 mg, 15%). MS (ESI) calculated for C₆H₇BN₂O₂: 138.06 m/z, found 138.95 [M+H]⁺.

### Example 61: N-[(1S)-5-[2-(2-aminopyridin-3-yl)-5-[1-(difluoromethyl)pyrazol-4-yl]imidazo[4,5-b]pyridin-3-yl]-2,3-dihydro-1H-inden-1-yl]-3-formyl-4-hydroxybenzamide

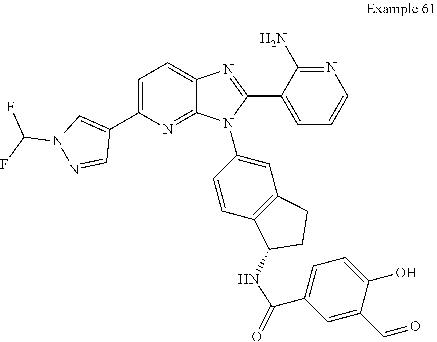 [SMILES: Nc1ncccc1-c1nc2ccc(-c3cnn(C(F)F)c3)nc2n1-c1ccc2c(c1)CC[C@@H]2NC(=O)c1ccc(O)c(C=O)c1]

Example 61 was prepared in a manner analogous to Example 19 using 1-(difluoromethyl)-4-(4,4,5,5-tetramethyl-1,3,2-dioxaborolan-2-yl)pyrazole in place of 3-(4-methylpiperazin-1-yl)phenylboronic acid, PyBOP in place of HATU and Intermediate 57-2 in place of Intermediate 5-1. MS (ESI) calculated for C₃₂H₂₄F₂N₈O₃: 606.19 m/z, found 607.15 [M+H]⁺. ¹H NMR (400 MHz, DMSO-d₆) δ (ppm): 10.30 (s, 1H), 8.71 (s, 1H), 8.24-8.33 (m, 3H), 8.10 (dd, J=8.7, 2.4 Hz, 1H), 8.05 (dd, J=5.8, 1.7 Hz, 1H), 7.81-7.90 (m, 2H), 7.63-7.71 (m, 1H), 7.34 (d, J=1.8 Hz, 1H), 7.30-7.36 (m, 2H), 7.08 (d, J=8.7 Hz, 1H), 6.74 (dd, J=7.6, 5.8 Hz, 1H), 5.58-6.64 (m, 1H), 2.99-3.10 (m, 1H), 2.89-2.95 (m, 1H), 2.48-2.51 (m, 1H), 2.09-2.13 (m, 1H). ¹⁹F NMR (376 MHz, DMSO-d₆) δ (ppm): −94.45.

### Example 62: N-[(1S)-5-[2-(2-aminopyridin-3-yl)-5-(1-cyclopropylpyrazol-4-yl)imidazo[4,5-b]pyridin-3-yl]-2,3-dihydro-1H-inden-1-yl]-3-formyl-4-hydroxybenzamide

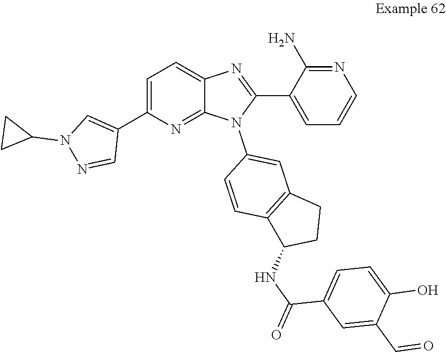 [SMILES: Nc1ncccc1-c1nc2ccc(-c3cnn(C4CC4)c3)nc2n1-c1ccc2c(c1)CC[C@@H]2NC(=O)c1ccc(O)c(C=O)c1]

Example 62 was prepared in a manner analogous to Example 19 using 1-cyclopropyl-4-(4,4,5,5-tetramethyl-1,3,2-dioxaborolan-2-yl)pyrazole in place of 3-(4-methylpiperazin-1-yl)phenylboronic acid, PyBOP in place of HATU and Intermediate 57-2 in place of Intermediate 5-1. MS (ESI) calculated for C₃₄H₂₈N₈O₃: 596.23 m/z, found 597.20 [M+H]⁺. ¹H NMR (400 MHz, DMSO-d₆) δ (ppm): 10.31 (s, 1H), 8.30-8.33 (m, 1H), 8.25-8.30 (m, 1H), 8.18-8.22 (m, 1H), 8.08-8.11 (m, 1H), 8.01-8.05 (m, 1H), 7.90 (s, 1H), 7.70-7.75 (m, 1H), 7.60-7.66 (m, 1H), 7.40-7.45 (m, 1H), 7.34-7.38 (m, 1H), 7.27-7.31 (m, 1H), 7.07-7.11 (m, 1H), 6.72-6.77 (m, 1H), 5.58-5.65 (m, 1H), 3.70-3.85 (m, 1H), 3.00-3.11 (m, 1H), 2.85-2.97 (m, 1H), 2.48-2.52 (m, 1H), 2.07-2.13 (m, 1H), 1.02-1.10 (m, 2H), 0.93-1.02 (m, 2H).

### Example 63: (S)—N-(5-(2-(2-aminopyridin-3-yl)-5-phenyl-3H-imidazo[4,5-b]pyridin-3-yl)-2,3-dihydro-1H-inden-1-yl)-4-(3,3-dimethylureido)-3-formylbenzamide

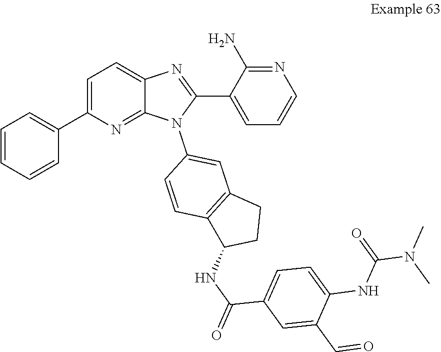 [SMILES: CN(C)C(=O)Nc1ccc(C(=O)N[C@H]2CCc3cc(-n4c(-c5cccnc5N)nc5ccc(-c6ccccc6)nc54)ccc32)cc1C=O]

Example 63 was prepared in a manner analogous to Example 12 using Intermediate 63-2 in place of Intermediate 12-1 and dichloromethane/2,2,2-trifluoroacetic acid (10:1) in place of methanesulfonic acid/2,2,2-trifluoroacetic acid. MS (ESI) calculated for C₃₇H₃₂N₈O₃: 636.26 m/z, found 637.30 [M+H]⁺. ¹H NMR (400 MHz, DMSO-d₆) δ (ppm): 9.99 (s, 1H), 8.45-8.51 (m, 2H), 8.24-8.26 (m, 1H), 8.15-8.17 (m, 1H), 7.95-8.01 (m, 4H), 7.44-7.48 (m, 2H), 7.37-7.41 (m, 3H), 7.25-7.32 (m, 2H), 6.44-6.47 (m, 1H), 5.63-5.67 (m, 1H), 3.01-3.06 (m, 7H), 2.86-2.95 (m, 1H), 2.57 (s, 1H), 2.05-2.14 (m, 1H).

### Intermediate 63-2: 4-(3,3-dimethylureido)-3-(1,3-dioxolan-2-yl)benzoic acid

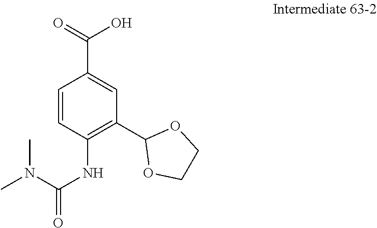 [SMILES: CN(C)C(=O)Nc1ccc(C(=O)O)cc1C1OCCO1]

### Synthetic Route:

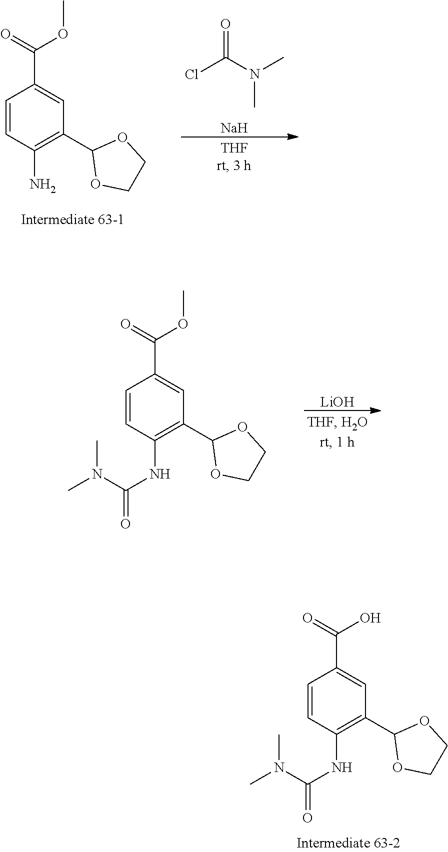

### Step 1: Synthesis of methyl 4-[(dimethylcarbamoyl)amino]-3-(1,3-dioxolan-2-yl)benzoate

To a cooled (0° C.) solution of methyl 4-amino-3-(1,3-dioxolan-2-yl)benzoate (Intermediate 63-1) (400 mg, 1.79 mmol, 1 equiv) in tetrahydrofuran (20 mL) was added 60% sodium hydride (86 mg, 2.15 mmol, 1.2 equiv). The mixture was stirred for 15 min then dimethylcarbamoyl chloride (385 mg, 3.58 mmol, 2 equiv) was added. The resulting mixture was stirred for 1 h at room temperature. The reaction mixture was quenched by the addition of water and extracted with ethyl acetate (3×25 mL). The combined organic layers were concentrated in vacuo and the resulting residue was purified by reverse-phase flash column chromatography on C18 silica gel using a 20-95% gradient of acetonitrile in water (+0.05% ammonium bicarbonate) with a 10-minute hold at 70% acetonitrile to afford methyl 4-[(dimethylcarbamoyl)amino]-3-(1,3-dioxolan-2-yl)benzoate (170 mg, 32%) as a white solid. MS (ESI) calculated for C₁₄H₁₈N₂O₅: 294.12 m/z, found 295.10 [M+H]⁺.

### Step 2: Synthesis of 4-[(dimethylcarbamoyl)amino]-3-(1,3-dioxolan-2-yl)benzoic acid (Intermediate 63-2)

To a solution of methyl 4-[(dimethylcarbamoyl)amino]-3-(1,3-dioxolan-2-yl)benzoate (170 mg, 0.578 mmol, 1 equiv) in tetrahydrofuran (10 mL) and water (5 mL) was added 2M aqueous lithium hydroxide (0.58 mL, 1.16 mmol, 2 equiv). The resulting mixture was stirred at room temperature for 1 h and then purified by reverse-phase flash column chromatography on C18 silica gel using a 20-95% gradient of acetonitrile in water (+0.05% ammonium bicarbonate) with a 10 minute hold at 70% acetonitrile to afford 4-[(dimethylcarbamoyl)amino]-3-(1,3-dioxolan-2-yl)benzoic acid (Intermediate 63-2) (90 mg, 56%) as a white solid. MS (ESI) calculated for C₁₃H₁₆N₂O₅: 280.11 m/z, found 281.05 [M+H]⁺.

### Intermediate 63-1: methyl 4-amino-3-(1,3-dioxolan-2-yl)benzoate

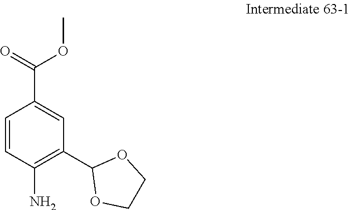 [SMILES: COC(=O)c1ccc(N)c(C2OCCO2)c1]

### Synthetic Route:

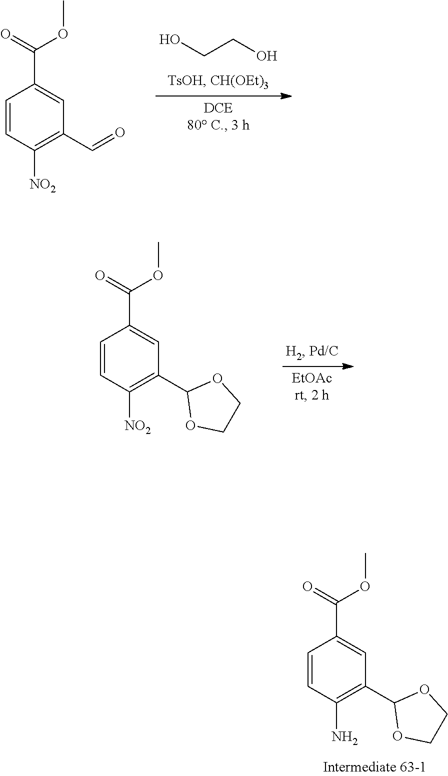

### Step 1: Synthesis of methyl 3-(1,3-dioxolan-2-yl)-4-nitrobenzoate

To a solution of methyl 3-formyl-4-nitrobenzoate (3.00 g, 14.3 mmol, 1 equiv) in 1,2-dichloroethane (30 mL) was added ethylene glycol (4.45 g, 71.7 mmol, 5 equiv), p-toluenesulfonic acid (1.23 g, 7.17 mmol, 0.5 equiv) and triethylorthoformate (6.38 g, 43.0 mmol, 3 equiv). The resulting mixture was stirred at 80° C. for 3 h. The reaction mixture was cooled to room temperature and concentrated in vacuo. The residue obtained was purified by silica gel column chromatography using a 0-10% gradient of ethyl acetate in petroleum ether) to afford methyl 3-(1,3-dioxolan-2-yl)-4-nitrobenzoate (2.87 g, 79%) as a yellow oil. MS (ESI) calculated for C₁₁H₁₁NO₆: 253.06 m/z, found 254.05 [M+H]⁺.

### Step 2: Synthesis of methyl 4-amino-3-(1,3-dioxolan-2-yl)benzoate (Intermediate 63-1)

To a solution of methyl 3-(1,3-dioxolan-2-yl)-4-nitrobenzoate (2.86 g, 11.3 mmol, 1 equiv) in ethyl acetate (100 mL) was added 10% palladium on carbon (1.4 g, 13 mmol, 1.15 equiv). The mixture was stirred at room temperature under hydrogen atmosphere for 1 h then filtered through celite and concentrated under reduced pressure to afford methyl 4-amino-3-(1,3-dioxolan-2-yl)benzoate (Intermediate 63-1) (2.3 g, 91%) as a yellow oil, which was used directly in subsequent transformations without further purification. MS (ESI) calculated for C₁₁H₁₃NO₄: 223.08 m/z, found 224.15 [M+H]⁺.

### Example 64: N-[(1S)-5-[2-(2-aminopyridin-3-yl)-5-phenylimidazo[4,5-b]pyridin-3-yl]-2,3-dihydro-1H-inden-1-yl]-4-acetamido-3-fluoro-5-formylbenzamide

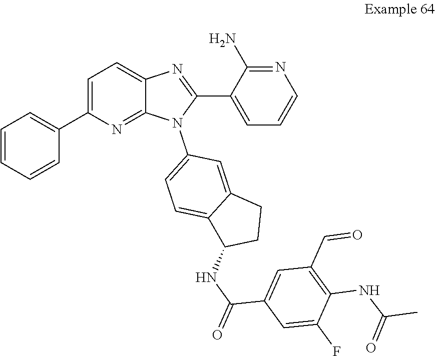 [SMILES: CC(=O)Nc1c(F)cc(C(=O)N[C@H]2CCc3cc(-n4c(-c5cccnc5N)nc5ccc(-c6ccccc6)nc54)ccc32)cc1C=O]

Example 64 was prepared in a manner analogous to Example 12 using Intermediate 64-1 in place of Intermediate 12-1. MS (ESI) calculated for C₃₆H₂₈FN₇O₃: 625.22 m/z, found 626.25 [M+H]⁺. ¹H NMR (400 MHz, DMSO-d₆) δ (ppm): 10.01 (s, 1H), 8.25-8.27 (m, 1H), 8.21-8.22 (m, 1H), 8.12-8.13 (m, 1H), 8.01-8.01 (m, 3H), 7.96-7.99 (m, 1H), 7.40-7.49 (m, 2H), 7.32-7.34 (m, 3H), 7.27-7.28 (m, 1H), 7.25-7.26 (m, 1H), 6.43-6.47 (m, 1H), 5.63-5.67 (m, 1H), 3.42-3.44 (m, 1H), 2.93-2.94 (m, 1H), 2.54-2.55 (m, 1H), 2.13-2.17 (m, 3H), 2.10-2.11 (m, 1H). ¹⁹F NMR (400 MHz, DMSO-d₆) δ (ppm): −120.92.

### Intermediate 64-1: 4-acetamido-3-(1,3-dioxolan-2-yl)-5-fluorobenzoic acid

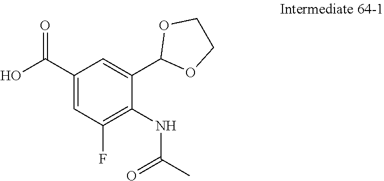 [SMILES: CC(=O)Nc1c(F)cc(C(=O)O)cc1C1OCCO1]

Intermediate 64-1 was prepared in a manner analogous to Intermediate 45-1 starting from 2-amino-5-bromo-3-fluorobenzaldehyde instead of 2-amino-5-bromobenzaldehyde. The crude Intermediate 45-1 obtained was purified by reverse-phase flash column chromatography on C18 silica gel using a 20-95% gradient of acetonitrile in water (+0.05% ammonium bicarbonate) with a 5 min hold at 50% acetonitrile to afford 3-(1,3-dioxolan-2-yl)-4-acetamido-5-fluorobenzoic acid (Intermediate 64-1). MS (ESI) calculated for C₁₂H₁₂FNO₅: 269.07 m/z, found 268.10 [M−H]⁻.

### Example 65: N-[(1S)-5-[2-(2-aminopyridin-3-yl)-5-(5-methylpyrazin-2-yl)imidazo[4,5-b]pyridin-3-yl]-2,3-dihydro-1H-inden-1-yl]-3-formyl-4-hydroxybenzamide

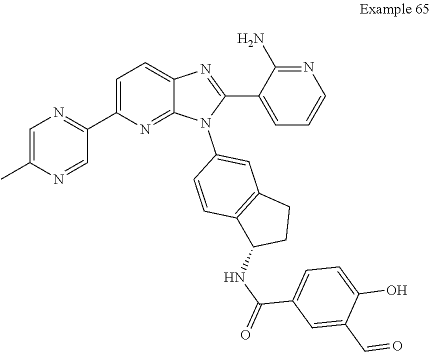 [SMILES: Cc1cnc(-c2ccc3nc(-c4cccnc4N)n(-c4ccc5c(c4)CC[C@@H]5NC(=O)c4ccc(O)c(C=O)c4)c3n2)cn1]

Example 65 was prepared in a manner analogous to Example 19 using Intermediate 65-1 in place of 3-(4-methylpiperazin-1-yl)phenylboronic acid and PyBOP in place of HATU. MS (ESI) calculated for C₃₃H₂₆N₈O₃: 582.21 m/z, found 583.20 [M+H]⁺. ¹H NMR (400 MHz, DMSO-d₆) δ (ppm): 10.26-10.28 (m, 1H), 9.17-9.18 (m, 1H), 8.60-8.61 (m, 1H), 8.29-8.36 (m, 2H), 8.28-8.29 (m, 1H), 8.07-8.09 (m, 1H), 8.02-8.03 (m, 1H), 7.52-7.54 (m, 1H), 7.43-7.45 (m, 1H), 7.33-7.38 (m, 2H), 7.06-7.08 (m, 1H), 6.62-6.64 (m, 1H), 5.61-5.63 (m, 1H), 3.02-3.04 (m, 1H), 2.88-2.92 (m, 1H), 2.50-2.53 (m, 3H), 2.36-2.37 (m, 1H), 2.06-2.09 (m, 1H).

### Intermediate 65-1: (5-methylpyrazin-2-yl)boronic acid

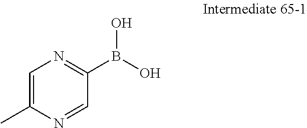

Intermediate 65-1 was prepared in manner analogous to Intermediate 60-1 using 2-bromo-5-methylpyrazine in place of 2-bromo-6-methylpyrazine. MS (ESI) calculated for C₅H₇BN₂O₂: 138.06 m/z, found 139.10 [M+H]⁺.

### Example 66: N-[(1S)-5-[2-(2-aminopyridin-3-yl)-5-(pyrazol-1-yl)imidazo[4,5-b]pyridin-3-yl]-2,3-dihydro-1H-inden-1-yl]-7-formyl-1H-indazole-5-carboxamide

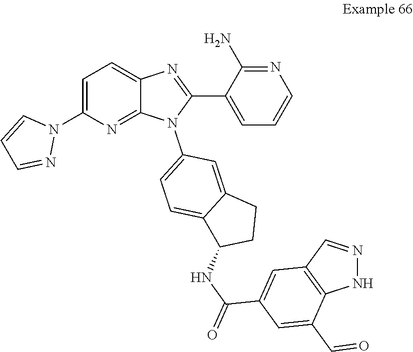 [SMILES: Nc1ncccc1-c1nc2ccc(-n3cccn3)nc2n1-c1ccc2c(c1)CC[C@@H]2NC(=O)c1cc(C=O)c2[nH]ncc2c1]

Example 66 was prepared in a manner analogous to Example 12 using Intermediate 33-1 in place of Intermediate 1-3 and Intermediate 35-1 in place of Intermediate 12-1. MS (ESI) calculated for C₃₂H₂₄N₁₀O₂: 580.21 m/z, found 581.20 [M+H]⁺. ¹H NMR (400 MHz, DMSO-d₆) δ (ppm): 10.20-10.21 (m, 1H), 8.75-8.76 (m, 1H), 8.59-8.61 (m, 1H), 8.33-8.39 (m, 1H), 8.32-8.34 (m, 2H), 8.00-8.01 (m, 1H), 7.94-7.99 (m, 1H), 7.78-7.92 (m, 1H), 7.38-7.40 (m, 2H), 7.26-7.29 (m, 2H), 6.53-6.54 (m, 1H), 6.44-6.48 (m, 1H), 5.65-5.69 (m, 1H), 3.02-3.04 (m, 1H), 2.92-2.94 (m, 1H), 2.57-2.58 (m, 1H), 2.12-2.15 (m, 1H).

### Example 67: N-[(1S)-5-[2-(2-aminopyridin-3-yl)-5-(1H-pyrazol-4-yl)imidazo[4,5-b]pyridin-3-yl]-2,3-dihydro-1H-inden-1-yl]-3-formyl-4-hydroxybenzamide

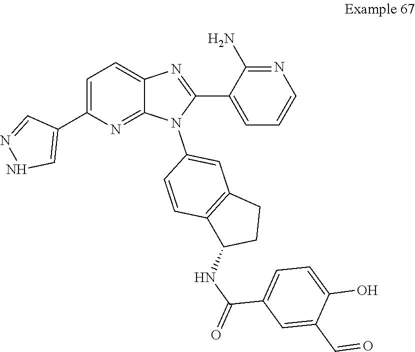 [SMILES: Nc1ncccc1-c1nc2ccc(-c3cn[nH]c3)nc2n1-c1ccc2c(c1)CC[C@@H]2NC(=O)c1ccc(O)c(C=O)c1]

Example 67 was prepared in a manner analogous to Example 19 using 4-(4,4,5,5-tetramethyl-1,3,2-dioxaborolan-2-yl)-1H-pyrazole in place of 3-(4-methylpiperazin-1-yl)phenylboronic acid, PyBOP in place of HATU and Intermediate 57-2 in place of Intermediate 5-1. MS (ESI) calculated for C₃₁H₂₄N₈O₃: 556.20 m/z, found 557.25 [M+H]⁺. ¹H NMR (400 MHz, DMSO-d₆) δ (ppm): 12.98-13.01 (m, 1H), 10.31 (s, 1H), 8.91-8.93 (m, 1H), 8.30-8.32 (m, 1H), 8.16-8.22 (m, 1H), 8.14-8.15 (m, 1H), 8.07-8.09 (m, 1H), 7.99-8.00 (m, 2H), 7.70-7.72 (m, 1H), 7.34-7.38 (m, 1H), 7.25-7.35 (m, 1H), 7.22-7.24 (m, 2H), 7.01-7.03 (m, 3H), 6.42-6.45 (m, 1H), 5.63-5.65 (m, 1H), 3.01-3.03 (m, 1H), 2.89-2.93 (m, 1H), 2.32-2.51 (m, 1H), 2.07-2.10 (m, 1H).

### Example 68: N-(4-{[(1S)-5-[2-(2-aminopyridin-3-yl)-5-phenylimidazo[4,5-b]pyridin-3-yl]-2,3-dihydro-1H-inden-1-yl]carbamoyl}-2-formylphenyl)morpholine-4-carboxamide

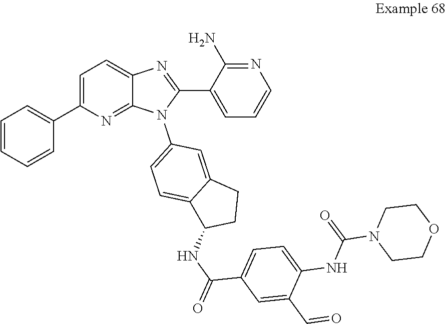 [SMILES: Nc1ncccc1-c1nc2ccc(-c3ccccc3)nc2n1-c1ccc2c(c1)CC[C@@H]2NC(=O)c1ccc(NC(=O)N2CCOCC2)c(C=O)c1]

Example 68 was prepared in a manner analogous to Example 12 using Intermediate 68-1 in place of Intermediate 12-1 and dichloromethane/2,2,2-trifluoroacetic acid (10:1) in place of 2,2,2-trifluoroacetic acid/methanesulfonic acid. MS (ESI) calculated for C₃₉H₃₄N₈O₄: 678.27 m/z, found 679.30 [M+H]⁺. ¹H NMR (400 MHz, DMSO-d₆) δ (ppm): 9.95-9.96 (m, 1H), 8.37-8.42 (m, 2H), 8.23-8.25 (m, 1H), 8.13-8.16 (m, 1H), 7.94-7.99 (m, 4H), 7.44-7.47 (m, 2H), 7.36-7.40 (m, 3H), 7.26-7.31 (m, 2H), 6.45-6.48 (m, 1H), 5.61-5.63 (m, 1H), 3.65 (s, 4H), 3.47 (s, 4H), 3.00-3.02 (m, 1H), 2.88-2.91 (m, 1H), 2.51 (s, 1H), 2.06-2.10 (m, 1H).

### Intermediate 68-1: 3-(1,3-dioxolan-2-yl)-4-(morpholine-4-carboxamido)benzoic acid

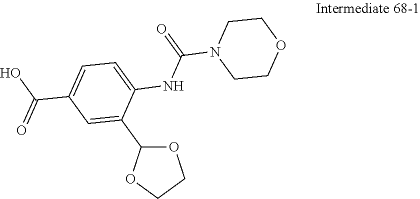 [SMILES: O=C(O)c1ccc(NC(=O)N2CCOCC2)c(C2OCCO2)c1]

### Synthetic Route:

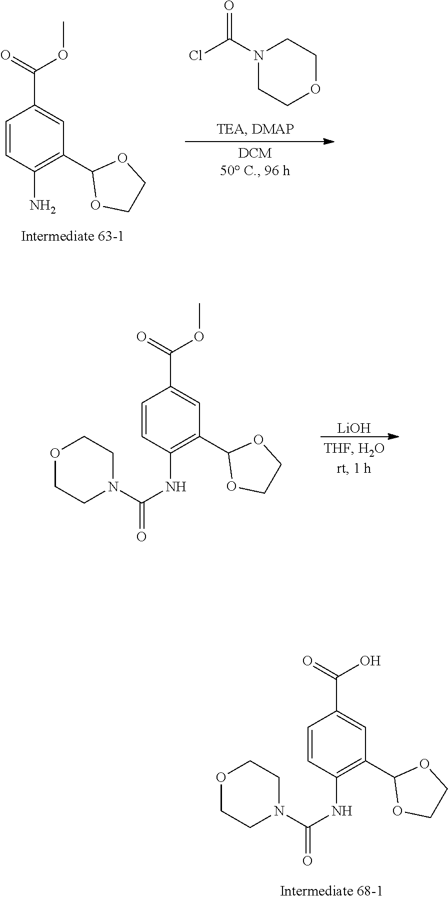 [SMILES: COC(=O)c1ccc(N)c(C2OCCO2)c1 ; O=C(Cl)N1CCOCC1 ; COC(=O)c1ccc(NC(=O)N2CCOCC2)c(C2OCCO2)c1 ; "LiOH" ; O=C(O)c1ccc(NC(=O)N2CCOCC2)c(C2OCCO2)c1]

### Step 1: Synthesis of methyl 3-(1,3-dioxolan-2-yl)-4-(morpholine-4-carbonylamino)benzoate

To a solution of methyl 4-amino-3-(1,3-dioxolan-2-yl)benzoate (Intermediate 63-1) (600 mg, 2.69 mmol, 1 equiv) in dichloromethane (20 mL) was added triethylamine (1.09 mg, 10.8 mmol, 4 equiv), 4-dimethylaminopyridine (328 mg, 2.69 mmol, 1 equiv) and morpholine-4-carbonyl chloride (603 mg, 4.03 mmol, 1.5 equiv). The resulting mixture was stirred at 50° C. for 4 days then concentrated in vacuo. The resulting residue was purified by reverse-phase flash column chromatography on C18 silica gel using a 20-95% gradient of acetonitrile in water (+0.05% ammonium bicarbonate) with a 10-minute hold at 70% acetonitrile to afford methyl 3-(1,3-dioxolan-2-yl)-4-(morpholine-4-carbonylamino)benzoate (70 mg, 7.74%) as a white solid. MS (ESI) calculated for C₁₆H₂₀N₂O₆: 336.13 m/z, found 337.10 [M+H]⁺.

### Step 2: Synthesis of 3-(1,3-dioxolan-2-yl)-4-(morpholine-4-carbonylamino)benzoic acid (Intermediate 68-1)

To a solution of methyl 3-(1,3-dioxolan-2-yl)-4-(morpholine-4-carbonylamino)benzoate (70 mg, 0.21 mmol, 1 equiv) in tetrahydrofuran (5 mL) and water (2 mL) was added 2M aqueous lithium hydroxide (0.21 mL, 0.42 mmol, 2 equiv). The resulting mixture was stirred at room temperature for 1 h. The solvent was removed under reduced pressure to afford crude 3-(1,3-dioxolan-2-yl)-4-(morpholine-4-carbonylamino)benzoic acid (Intermediate 68-1) (60 mg, crude) as a white solid, which was used without further purification in subsequent transformations. MS (ESI) calculated for C₁₅H₁₈N₂O₆: 322.12 m/z, found 323.00 [M+H]⁺.

### Example 69: N-[(1S)-5-[2-(2-aminopyridin-3-yl)-5,7-dimethylimidazo[4,5-b]pyridin-3-yl]-2,3-dihydro-1H-inden-1-yl]-3-formyl-4-hydroxybenzamide

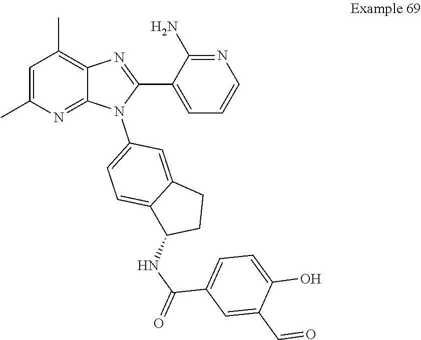 [SMILES: Cc1cc(C)c2nc(-c3cccnc3N)n(-c3ccc4c(c3)CC[C@@H]4NC(=O)c3ccc(O)c(C=O)c3)c2n1]

Example 69 was prepared in a manner analogous to Example 12 using Intermediate 69-1 in place of Intermediate 1-3 and Intermediate 57-2 in place of Intermediate 12-1. MS (ESI) calculated for C₃₀H₂₆N₆O₃: 518.21 m/z, found 519.30 [M+H]⁺. ¹H NMR (400 MHz, DMSO-d₆) δ (ppm): 10.20-10.44 (m, 1H), 8.20-8.40 (m, 1H), 8.00-8.10 (m, 1H), 7.90-8.00 (m, 1H), 7.40-7.50 (m, 2H), 7.15-7.40 (m, 2H), 7.00-7.10 (m, 1H), 6.85-7.00 (m, 1H), 6.30-6.50 (m, 1H), 5.50-5.70 (m, 1H), 2.95-3.10 (m, 1H), 2.80-2.95 (m, 1H), 2.60-2.70 (m, 3H), 2.40-2.50 (m, 4H), 2.00-2.15 (m, 1H).

### Intermediate 69-1: (S)-3-(3-(1-amino-2,3-dihydro-1H-inden-5-yl)-5,7-dimethyl-3H-imidazo[4,5-b]pyridin-2-yl)pyridin-2-amine

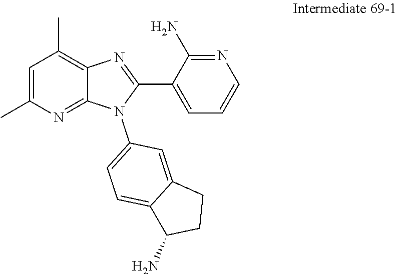 [SMILES: Cc1cc(C)c2nc(-c3cccnc3N)n(-c3ccc4c(c3)CC[C@@H]4N)c2n1]

### Synthetic Route:

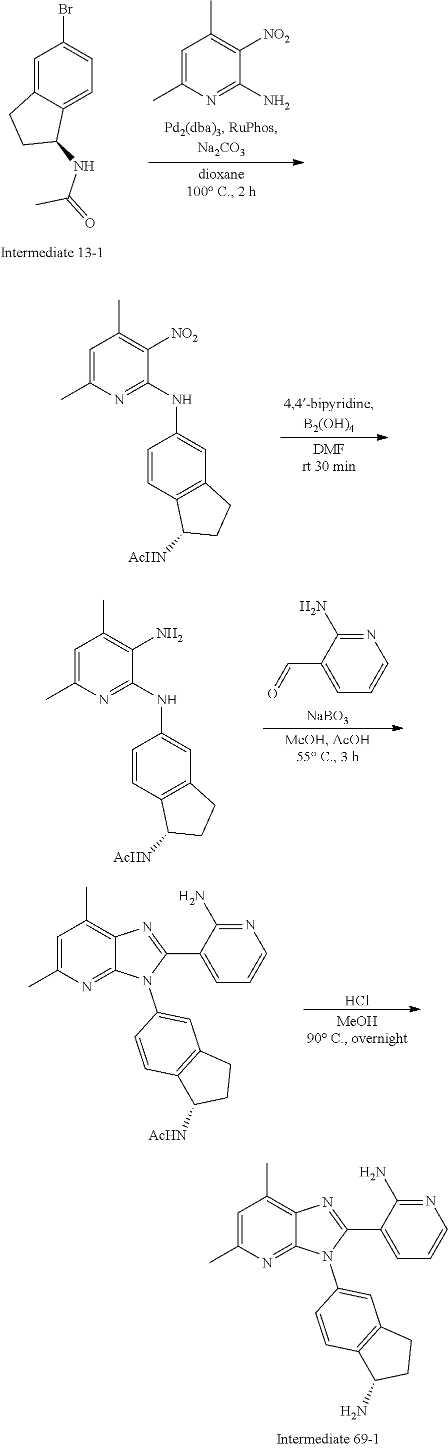

### Step 1: Synthesis of N-[(1S)-5-[(4,6-dimethyl-3-nitropyridin-2-yl)amino]-2,3-dihydro-1H-inden-1-yl]acetamide

To a solution of N-[(1S)-5-bromo-2,3-dihydro-1H-inden-1-yl]acetamide (Intermediate 13-1) (1.00 g, 3.94 mmol, 1 equiv), 4,6-dimethyl-3-nitropyridin-2-amine (0.66 g, 3.9 mmol, 1 equiv) and RuPhos (0.37 g, 0.79 mmol, 0.2 equiv) in 1,4-dioxane (15 mL) and were added sodium carbonate (1.25 g, 11.8 mmol, 3 equiv) and tris(dibenzylideneacetone)dipalladium(0) (0.36 g, 0.39 mmol, 0.1 equiv). After stirring for 2 h at 100° C. under a nitrogen atmosphere, the resulting mixture was concentrated under reduced pressure and the residue was purified by reverse-phase flash column chromatography on C18 silica gel using a 10-50% gradient of acetonitrile in water (+0.05% ammonium bicarbonate) to afford N-[(1S)-5-[(4,6-dimethyl-3-nitropyridin-2-yl)amino]-2,3-dihydro-1H-inden-1-yl]acetamide (530 mg, 39%) as a yellow solid. MS (ESI) calculated for C₁₈H₂₀N₄O₃: 340.15 m/z, found 341.15 [M+H]⁺.

### Step 2: Synthesis of N-[(1S)-5-[(3-amino-4,6-dimethylpyridin-2-yl)amino]-2,3-dihydro-1H-inden-1-yl]acetamide

To a solution of N-[(1S)-5-[(4,6-dimethyl-3-nitropyridin-2-yl)amino]-2,3-dihydro-1H-inden-1-yl]acetamide (450 mg, 1.32 mmol, 1 equiv) in N,N-dimethylformamide (5 mL) was added hypodiboric acid (356 mg, 3.97 mmol, 3 equiv) and 4,4′-bipyridine (10 mg, 0.066 mmol, 0.05 equiv). The resulting mixture was stirred at room temperature for 30 min. Water was added and the mixture was extracted with ethyl acetate (3×100 mL). The combined organic extracts were concentrated under reduced pressure to afford N-[(1S)-5-[(3-amino-4,6-dimethylpyridin-2-yl)amino]-2,3-dihydro-1H-inden-1-yl]acetamide (300 mg, 64%) as a yellow solid, which was used without further purification in the next step. MS (ESI) calculated for C₁₈H₂₂N₄O: 310.18 m/z, found 311.15 [M+H]⁺.

### Step 3: Synthesis of N-[(1S)-5-[2-(2-aminopyridin-3-yl)-5,7-dimethylimidazo[4,5-b]pyridin-3-yl]-2,3-dihydro-1H-inden-1-yl]acetamide

To a solution of N-[(1S)-5-[(3-amino-4,6-dimethylpyridin-2-yl)amino]-2,3-dihydro-1H-inden-1-yl]acetamide (300 mg, 0.966 mmol, 1 equiv) in acetic acid (12.5 mL) and methanol (1.5 mL) were added 2-aminopyridine-3-carbaldehyde (142 mg, 1.16 mmol, 1.2 equiv) and sodium perborate (178 mg, 2.17 mmol, 2.25 equiv). The resulting mixture was stirred at 55° C. for 3 h then cooled to room temperature and concentrated under reduced pressure. The residue was purified by reverse-phase flash column chromatography on C18 silica gel using a 10-50% gradient of acetonitrile in water (+0.05% ammonium bicarbonate) to afford N-[(1S)-5-[2-(2-aminopyridin-3-yl)-5,7-dimethylimidazo[4,5-b]pyridin-3-yl]-2,3-dihydro-1H-inden-1-yl]acetamide (120 mg, 28%) as a yellow solid. MS (ESI) calculated for C₂₄H₂₄N₆O: 412.20 m/z, found 413.20 [M+H]⁺.

### Step 4: Synthesis of 3-{3-[(1S)-1-amino-2,3-dihydro-1H-inden-5-yl]-5,7-dimethylimidazo[4,5-b]pyridin-2-yl}pyridin-2-amine (Intermediate 69-1)

N-[(1S)-5-[2-(2-aminopyridin-3-yl)-5,7-dimethylimidazo[4,5-b]pyridin-3-yl]-2,3-dihydro-1H-inden-1-yl]acetamide (120 mg, 0.291 mmol, 1 equiv) was dissolved in hydrochloric acid (10 mL, concentrated) and methanol (10 mL) and the resulting mixture was stirred at 90° C. overnight. The mixture was concentrated under reduced pressure to afforded 3-{3-[(1S)-1-amino-2,3-dihydro-1H-inden-5-yl]-5,7-dimethylimidazo[4,5-b]pyridin-2-yl}pyridin-2-amine (Intermediate 69-1) (100 mg, 85%) as a brown solid, which was used in subsequent transformations without further purification. MS (ESI) calculated for C₂₂H₂₂N₆: 370.19 m/z, found 371.15 [M+H]⁺.

### Example 70: (S)—N-(5-(2-(2-aminopyridin-3-yl)-5-(pyrazin-2-yl)-3H-imidazo[4,5-b]pyridin-3-yl)-2,3-dihydro-1H-inden-1-yl)-3-fluoro-5-formyl-4-hydroxybenzamide

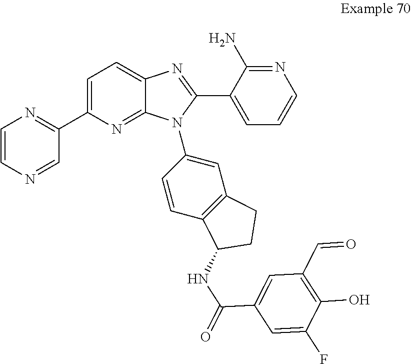 [SMILES: Nc1ncccc1-c1nc2ccc(-c3cnccn3)nc2n1-c1ccc2c(c1)CC[C@@H]2NC(=O)c1cc(F)c(O)c(C=O)c1]

Example 70 was prepared in a manner analogous to Example 19 using pyrazin-2-ylboronic acid in place of 3-(4-methylpiperazin-1-yl)phenylboronic acid, Intermediate 44-1 in place of Intermediate 5-1 and PyBOP in place of HATU. MS (ESI) calculated for C₃₂H₂₃FN₈O₃: 586.19 m/z, found 587.25 [M+H]⁺. ¹H NMR (400 MHz, DMSO-d₆) δ (ppm): 10.21 (s, 1H), 9.30-9.35 (m, 1H), 8.70-8.73 (m, 1H), 8.41-8.46 (m, 2H), 8.03-8.04 (m, 1H), 7.42-7.58 (m, 2H), 7.36-7.38 (m, 1H), 7.24-7.28 (m, 2H), 7.64-7.67 (m, 2H), 5.59-5.61 (m, 1H), 2.89-2.90 (m, 1H), 2.85-2.88 (m, 1H), 2.50-2.51 (m, 1H), 2.02-2.07 (m, 1H). ¹⁹F NMR (376 MHz, DMSO-d₆) δ (ppm): −73.4.

### Example 71: (S)—N-(5-(2-(2-aminopyridin-3-yl)-5-(5-methoxypyrazin-2-yl)-3H-imidazo[4,5-b]pyridin-3-yl)-2,3-dihydro-1H-inden-1-yl)-3-formyl-4-hydroxybenzamide

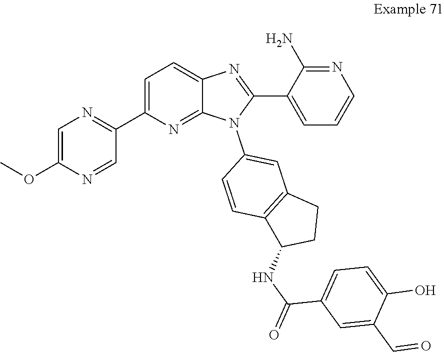 [SMILES: COc1cnc(-c2ccc3nc(-c4cccnc4N)n(-c4ccc5c(c4)CC[C@@H]5NC(=O)c4ccc(O)c(C=O)c4)c3n2)cn1]

Example 71 was prepared in a manner analogous to Example 12 using Intermediate 71-2 in place of Intermediate 1-3 and Intermediate 5-1 in place of Intermediate 12-1. MS (ESI) calculated for C₃₃H₂₆N₈O₄: 598.21 m/z, found 599.15 [M+H]⁺. ¹H NMR (400 MHz, DMSO-d₆) δ (ppm): 10.27 (s, 1H), 8.88-8.89 (m, 1H), 8.26-8.37 (m, 4H), 8.04-8.09 (m, 2H), 7.74-7.75 (m, 1H), 7.45-7.46 (m, 1H), 7.33-7.38 (m, 2H), 7.06-7.08 (m, 1H), 6.76-6.80 (m, 1H), 5.60-5.64 (m, 1H), 3.92-3.94 (m, 3H), 3.02-3.06 (m, 1H), 2.88-2.92 (m, 1H), 2.49-2.51 (m, 1H), 2.06-2.09 (m, 1H).

### Intermediate 71-2: (S)-3-(3-(1-amino-2,3-dihydro-1H-inden-5-yl)-5-(5-methoxypyrazin-2-yl)-3H-imidazo[4,5-b]pyridin-2-yl)pyridin-2-amine

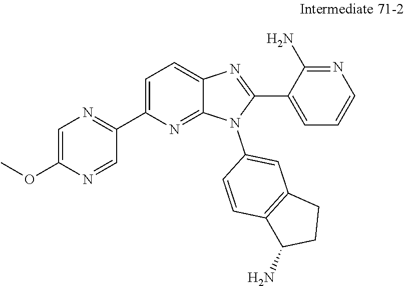 [SMILES: COc1cnc(-c2ccc3nc(-c4cccnc4N)n(-c4ccc5c(c4)CC[C@@H]5N)c3n2)cn1]

Intermediate 71-2 was prepared in a manner analogous to Intermediate 69-1 using Intermediate 71-1 in place of 4,6-dimethyl-3-nitropyridin-2-amine, Intermediate 71-3 in place of Intermediate 13-1 and 2,2,2-trifluoroacetic acid at room temperature for 2 h instead of hydrochloric acid/methanol at 90° C. overnight. MS (ESI) calculated for C₂₅H₂₂N₈O: 450.19 m/z, found 451.20 [M+H]⁺.

### Intermediate 71-1: 6-(5-methoxypyrazin-2-yl)-3-nitropyridin-2-amine

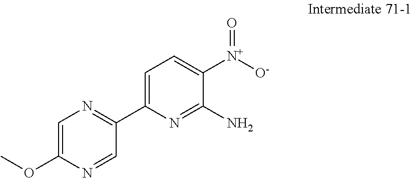 [SMILES: COc1cnc(-c2ccc([N+](=O)[O-])c(N)n2)cn1]

### Synthetic Route:

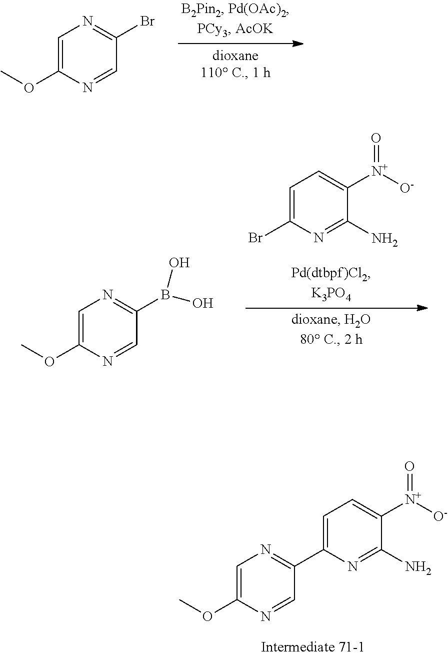

### Step 1: Synthesis of (5-methoxypyrazin-2-yl)boronic acid

To a solution of 2-bromo-5-methoxypyrazine (3.00 g, 15.9 mmol, 1 equiv) and bis(pinacolato)diboron (4.83 g, 19.0 mmol, 1.2 equiv) in 1,4-dioxane (30 mL) were added palladium(II) acetate (0.21 g, 0.95 mmol, 0.06 equiv), tricyclohexylphosphine (0.54 g, 1.9 mmol, 0.12 equiv) and potassium acetate (3.9 g, 40 mmol, 2.5 equiv). After stirring for 1 h at 110° C. under a nitrogen atmosphere the reaction mixture was concentrated in vacuo and the residue was purified by reverse-phase flash column chromatography on C18 silica gel using a 0-20% gradient of acetonitrile in water to afford 5-methoxypyrazin-2-ylboronic acid (2.1 g, 86%) as a yellow semi-solid. MS (ESI) calculated for C₅H₇BN₂O₃: 154.05 m/z, found 155.10 [M+H]⁺.

### Step 2: Synthesis of 6-(5-methoxypyrazin-2-yl)-3-nitropyridin-2-amine (Intermediate 71-1)

To a solution of 6-bromo-3-nitropyridin-2-amine (1.00 g, 5.76 mmol, 1 equiv) and 5-methoxypyrazin-2-ylboronic acid (1.77 g, 11.5 mmol, 2 equiv) in 1,4-dioxane (12 mL) and water (3 mL) were added bis(di-tert-butylphosphino)ferrocene]dichloropalladium(II) (0.38 g, 0.58 mmol, 0.1 equiv) and tribasic potassium phosphate (3.67 g, 17.3 mmol, 3 equiv). After stirring for 2 h at 80° C. under a nitrogen atmosphere, the reaction was cooled to 0° C. and quenched by the addition of saturated aqueous ammonium chloride (10 mL). The resulting mixture was extracted with ethyl acetate (50 mL×3). The combined organic layers were washed with water (30 mL×3), dried over anhydrous sodium sulfate, filtered, and concentrated under reduced pressure. The residue was purified by silica gel column chromatography eluting with 35% ethyl acetate in petroleum ether to afford 6-(5-methoxypyrazin-2-yl)-3-nitropyridin-2-amine (Intermediate 71-1) (0.8 g, 56%) as a yellow solid. MS (ESI) calculated for C₁₀H₉N₅O₃: 247.07 m/z, found 248.10 [M+H]⁺.

### Intermediate 71-3: tert-butyl (S)-(5-bromo-2,3-dihydro-1H-inden-1-yl)carbamate

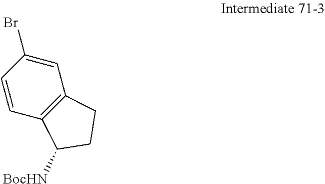 [SMILES: CC(C)(C)OC(=O)N[C@H]1CCc2cc(Br)ccc21]

### Synthetic Route:

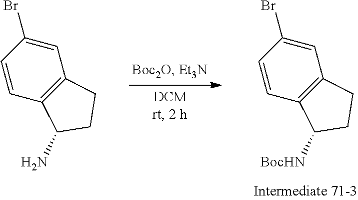 [SMILES: N[C@H]1CCc2cc(Br)ccc21 ; CC(C)(C)OC(=O)N[C@H]1CCc2cc(Br)ccc21]

### Step 1: Synthesis of tert-butyl (S)-(5-bromo-2,3-dihydro-1H-inden-1-yl)carbamate (Intermediate 71-3)

To a solution of (1S)-5-bromo-2,3-dihydro-1H-inden-1-amine (100 g, 471 mmol, 1 equiv) in dichloromethane (1 L) was added di-tert-butyl dicarbonate (154 g, 707 mmol, 1.5 equiv) followed by triethylamine (143 g, 1.41 mol, 3 equiv). The resulting mixture was stirred at room temperature for 2 h. The reaction mixture was filtered, rinsing with water (3×500 mL). The filtrate was extracted with dichloromethane (3×500 mL). The combined organic layers were washed with brine (1×100 mL), dried over anhydrous sodium sulfate, filtered, and concentrated in vacuo to afford tert-butyl N-[(1S)-5-bromo-2,3-dihydro-1H-inden-1-yl]carbamate (Intermediate 71-3) (150 g, 88%) as a white solid, which was used in subsequent transformations without further purification. MS (ESI) calculated for C₁₄H₁₈BrNO₂: 311.05 m/z, found 309.95 [M−H]⁻.

### Example 72: (S)—N-(5-(2-(2-aminopyridin-3-yl)-5-(1-methyl-1H-1,2,3-triazol-4-yl)-3H-imidazo[4,5-b]pyridin-3-yl)-2,3-dihydro-1H-inden-1-yl)-3-formyl-4-hydroxybenzamide

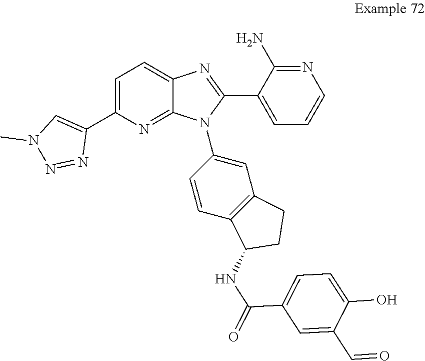 [SMILES: Cn1cc(-c2ccc3nc(-c4cccnc4N)n(-c4ccc5c(c4)CC[C@@H]5NC(=O)c4ccc(O)c(C=O)c4)c3n2)nn1]

Example 72 was prepared in a manner analogous to Example 12 using Intermediate 72-2 in place of Intermediate 1-3 and Intermediate 57-2 in place of Intermediate 12-1. MS (ESI) calculated for C₃₁H₂₅N₉O₃: 571.21 m/z, found 572.20 [M+H]⁺. ¹H NMR (400 MHz, DMSO-d₆) δ (ppm): 10.29 (s, 1H), 8.28-8.40 (m, 3H), 8.06-8.09 (m, 2H), 7.93-7.95 (m, 1H), 7.69-7.71 (m, 1H), 7.50-7.51 (m, 1H), 7.33-7.35 (m, 2H), 7.05-7.06 (m, 1H), 6.72-6.75 (m, 1H), 5.58-5.62 (m, 1H), 4.22 (s, 3H), 3.02-3.05 (m, 1H), 2.88-2.94 (m, 1H), 2.53-2.54 (m, 1H), 2.11-2.13 (m, 1H).

### Intermediate 72-2: (S)-3-(3-(1-amino-2,3-dihydro-1H-inden-5-yl)-5-(1-methyl-1H-1,2,3-triazol-4-yl)-3H-imidazo[4,5-b]pyridin-2-yl)pyridin-2-amine

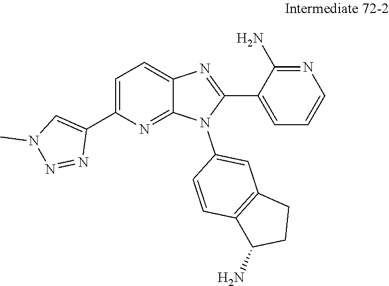 [SMILES: Cn1cc(-c2ccc3nc(-c4cccnc4N)n(-c4ccc5c(c4)CC[C@@H]5N)c3n2)nn1]

Intermediate 72-2 was prepared in a manner analogous to Intermediate 69-1 (starting from Step 2) using Intermediate 72-1 in place of the nitro starting material. MS (ESI) calculated for C₂₃H₂₁N₉: 423.19 m/z, found 424.20 [M+H]⁺.

### Intermediate 72-1: (S)—N-(5-((6-(1-methyl-1H-1,2,3-triazol-4-yl)-3-nitropyridin-2-yl)amino)-2,3-dihydro-1H-inden-1-yl)acetamide

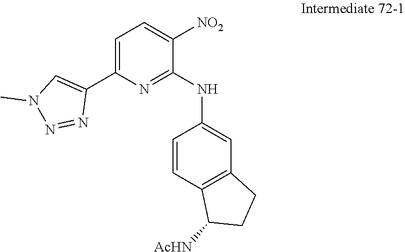 [SMILES: CC(=O)N[C@H]1CCc2cc(Nc3nc(-c4cn(C)nn4)ccc3[N+](=O)[O-])ccc21]

### Synthetic Route:

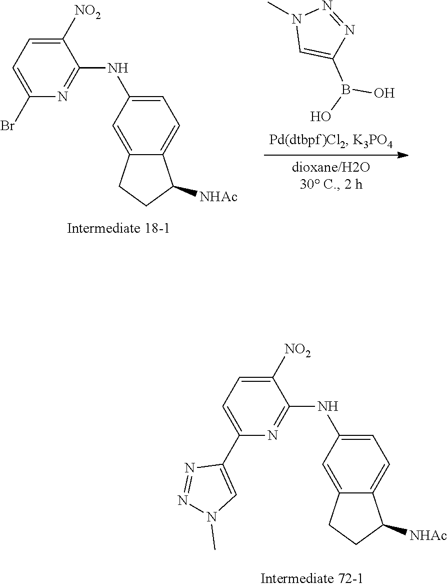

### Step 1: Synthesis of N-[(1S)-5-{[6-(1-methyl-1,2,3-triazol-4-yl)-3-nitropyridin-2-yl]amino}-2,3-dihydro-1H-inden-1-yl]acetamide (Intermediate 72-1)

To a solution of N-[(1S)-5-[(6-bromo-3-nitropyridin-2-yl)amino]-2,3-dihydro-1H-inden-1-yl]acetamide (Intermediate 18-1) (900 mg, 2.30 mmol, 1 equiv) in 1,4-dioxane (8 mL) and water (2 mL) was added 1-methyl-1,2,3-triazol-4-ylboronic acid (876 mg, 6.90 mmol, 3 equiv), bis(di-tert-butylphosphino)ferrocene]dichloropalladium(II) (150 mg, 0.230 mmol, 0.1 equiv) and tribasic potassium phosphate (1.46 g, 6.90 mmol, 3 equiv). The resulting mixture was stirred at 30° C. under nitrogen atmosphere for 2 h. The reaction mixture was concentrated in vacuo and the residue was purified by reverse-phase flash column chromatography on C18 silica gel using a 20-95% gradient of acetonitrile in water (+0.05% ammonium bicarbonate) with a 10 min hold at 70% acetonitrile to afford N-[(1S)-5-{[6-(1-methyl-1,2,3-triazol-4-yl)-3-nitropyridin-2-yl]amino}-2,3-dihydro-1H-inden-1-yl]acetamide (Intermediate 72-1) (900 mg, 66%) as a yellow solid. MS (ESI) calculated for C₁₉H₁₉N₇O₃: 393.15 m/z, found 394.10 [M+H]⁺.

### Example 73: (S)—N-(5-(2-(2-aminopyridin-3-yl)-5-(pyrazin-2-yl)-3H-imidazo[4,5-b]pyridin-3-yl)-2,3-dihydro-1H-inden-1-yl)-7-formyl-1H-indazole-5-carboxamide

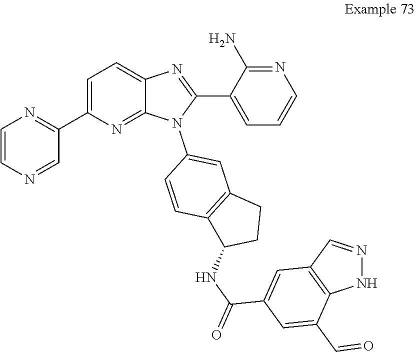 [SMILES: Nc1ncccc1-c1nc2ccc(-c3cnccn3)nc2n1-c1ccc2c(c1)CC[C@@H]2NC(=O)c1cc(C=O)c2[nH]ncc2c1]

Example 73 was prepared in a manner analogous to Example 12 using Intermediate 29-1 in place of Intermediate 1-3, Intermediate 35-1 in place of Intermediate 12-1 and omitting methanesulfonic acid. MS (ESI) calculated for C₃₃H₂₄N₁₀O₂: 592.21 m/z, found 593.20 [M+H]⁺. ¹H NMR (400 MHz, DMSO-d₆) δ (ppm): 10.22 (s, 1H), 9.33-9.34 (m, 1H), 8.78-8.79 (m, 1H), 8.72-8.73 (m, 1H), 8.64-8.65 (m, 2H), 8.37-8.41 (m, 3H), 8.02-8.03 (m, 1H), 7.44-7.45 (m, 2H), 7.31-7.33 (m, 2H), 6.47-6.49 (m, 1H), 5.71 (s, 1H), 2.94-3.07 (s, 2H), 2.50-2.51 (m, 1H), 2.13-2.15 (m, 1H).

### Example 74: N-[(1S)-5-[2-(2-aminopyridin-3-yl)-5-phenylimidazo[4,5-b]pyridin-3-yl]-3-oxo-1,2-dihydroinden-1-yl]-3-formyl-4-hydroxybenzamide

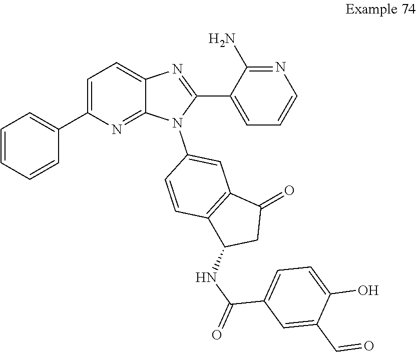 [SMILES: Nc1ncccc1-c1nc2ccc(-c3ccccc3)nc2n1-c1ccc2c(c1)C(=O)C[C@@H]2NC(=O)c1ccc(O)c(C=O)c1]

Example 74 was prepared in a manner analogous to Example 12 using Intermediate 74-2 in place of Intermediate 1-3 and Intermediate 57-2 in place of Intermediate 12-1. MS (ESI) calculated for C₃₄H₂₄N₆O₄: 580.19 m/z, found 581.20 [M+H]⁺. ¹H NMR (400 MHz, DMSO-d₆) δ (ppm): 10.30 (s, 1H), 8.30-8.40 (m, 1H), 8.20-8.30 (m, 1H), 7.90-8.20 (m, 5H), 7.80-7.90 (m, 2H), 7.75-7.80 (m, 1H), 7.65-7.75 (m, 1H), 7.30-7.60 (m, 3H), 6.90-7.20 (m, 1H), 6.60-6.90 (m, 1H), 5.55-6.05 (m, 1H), 3.20-3.30 (m, 1H), 2.70-2.80 (m, 1H).

### Intermediate 74-2: (S)-3-amino-6-(2-(2-aminopyridin-3-yl)-5-phenyl-3H-imidazo[4,5-b]pyridin-3-yl)-2,3-dihydro-1H-inden-1-one

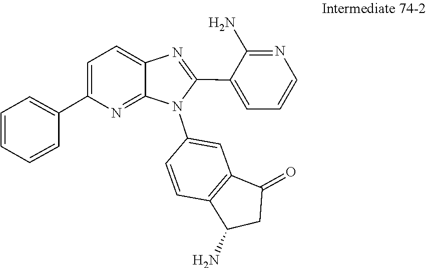 [SMILES: Nc1ncccc1-c1nc2ccc(-c3ccccc3)nc2n1-c1ccc2c(c1)C(=O)C[C@@H]2N]

Intermediate 74-2 was prepared in a manner analogous to Intermediate 69-1 using Intermediate 74-1 in place of Intermediate 13-1 and Pd₂(dba)₃/XantPhos/Cs₂CO₃ in place of Pd(OAc)₂/RuPhos/Na₂CO₃. MS (ESI) calculated for C₂₆H₂₀N₆O: 432.17 m/z, found 433.15 [M+H]⁺.

### Intermediate 74-1: (S)—N-(5-bromo-3-oxo-2,3-dihydro-1H-inden-1-yl)acetamide

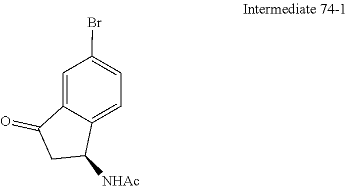 [SMILES: CC(=O)N[C@H]1CC(=O)c2cc(Br)ccc21]

### Synthetic Route:

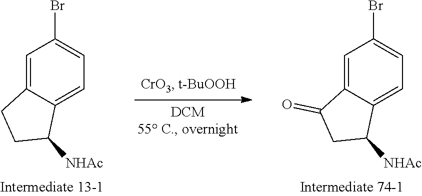 [SMILES: CC(=O)N[C@H]1CCc2cc(Br)ccc21 ; CC(=O)N[C@H]1CC(=O)c2cc(Br)ccc21]

### Step 1: Synthesis of N-[(1S)-5-bromo-3-oxo-1,2-dihydroinden-1-yl]acetamide (Intermediate 74-1)

To a solution of N-[(1S)-5-bromo-2,3-dihydro-1H-inden-1-yl]acetamide (Intermediate 13-1) (1.00 g, 3.94 mmol, 1 equiv) in dichloromethane (20 mL) was added chromium(VI) oxide (0.200 g, 1.97 mmol, 0.5 equiv) followed by tert-butyl hydroperoxide (5.05 g (70% aqueous), 39.4 mmol, 10 equiv). The resulting mixture was stirred at 50° C. overnight then cooled to room temperature and concentrated under reduced pressure. The residue was purified by silica gel column chromatography using a 0-20% gradient of methanol in dichloromethane to afford N-[(1S)-5-bromo-3-oxo-1,2-dihydroinden-1-yl]acetamide (Intermediate 74-1) (250 mg, 23%) as a white solid. MS (ESI) calculated for C₁₁H₁₀BrNO₂: 266.99 m/z, found 265.95 [M−H]⁻.

### Example 75: (S)—N-(5-(2-(2-aminopyridin-3-yl)-5-(3-fluoro-1H-pyrazol-1-yl)-3H-imidazo[4,5-b]pyridin-3-yl)-2,3-dihydro-1H-inden-1-yl)-3-formyl-4-hydroxybenzamide

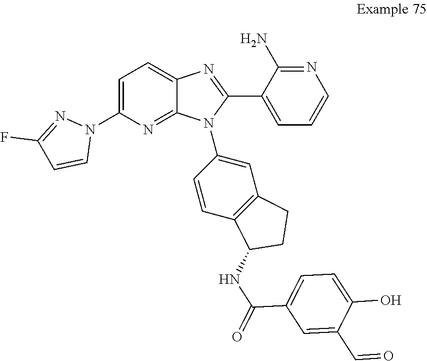 [SMILES: Nc1ncccc1-c1nc2ccc(-n3ccc(F)n3)nc2n1-c1ccc2c(c1)CC[C@@H]2NC(=O)c1ccc(O)c(C=O)c1]

Example 75 was prepared in a manner analogous Example 7 using Intermediate 75-1 in place of Intermediate 1-2 and Intermediate 57-2 in place of Intermediate 5-1. MS (ESI) calculated for C₃₁H₂₃FN₈O₃: 574.19 m/z, found 575.20 [M+H]⁺. ¹H NMR (400 MHz, DMSO-d₆) δ (ppm): 10.28 (s, 1H), 8.91-8.93 (m, 1H), 8.34-8.36 (m, 1H), 8.28-8.30 (m, 2H), 8.00-8.07 (m, 2H), 7.75-7.77 (m, 1H), 7.32-7.37 (m, 2H), 7.25-7.29 (m, 2H), 7.01-7.03 (m, 1H), 6.44-6.47 (m, 1H), 6.33-6.36 (m, 1H), 5.61-5.63 (m, 1H), 2.99-3.02 (m, 1H), 2.87-2.89 (m, 1H), 2.50-2.51 (m, 1H), 2.08-2.11 (m, 1H). ¹⁹F NMR (376 MHz, DMSO-d₆) δ (ppm): −127.27.

### Intermediate 75-1: (S)-3-(3-(1-amino-2,3-dihydro-1H-inden-5-yl)-5-(3-fluoro-1H-pyrazol-1-yl)-3H-imidazo[4,5-b]pyridin-2-yl)pyridin-2-amine

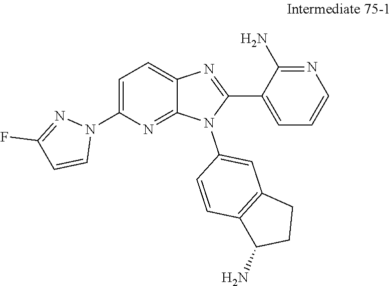 [SMILES: Nc1ncccc1-c1nc2ccc(-n3ccc(F)n3)nc2n1-c1ccc2c(c1)CC[C@@H]2N]

Intermediate 75-1 was prepared in a manner analogous to Intermediate 33-1 using 3-fluoro-1H-pyrazole in place of pyrazole. MS (ESI) calculated for C₂₃H₁₉FN₈: 426.17 m/z, found 427.10 [M+H]⁺.

### Example 76: N-[(1S)-5-[2-(2-aminopyridin-3-yl)-5-(4-fluoropyrazol-1-yl)imidazo[4,5-b]pyridin-3-yl]-2,3-dihydro-1H-inden-1-yl]-3-formyl-4-hydroxybenzamide

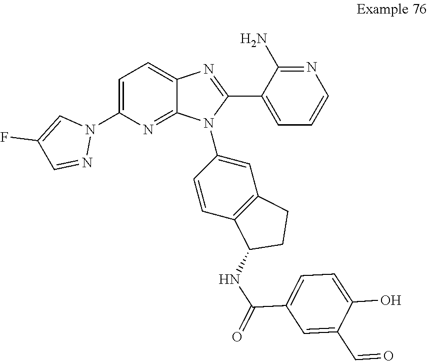 [SMILES: Nc1ncccc1-c1nc2ccc(-n3cc(F)cn3)nc2n1-c1ccc2c(c1)CC[C@@H]2NC(=O)c1ccc(O)c(C=O)c1]

Example 76 was prepared in a manner analogous to Example 12 using Intermediate 76-2 in place of Intermediate 1-3 and Intermediate 57-2 in place of Intermediate 12-1. MS (ESI) calculated for C₃₁H₂₃FN₈O₃: 574.19 m/z, found 575.25 [M+H]⁺. ¹H NMR (400 MHz, DMSO-d₆) δ (ppm): 10.17 (s, 1H), 8.33-8.46 (m, 2H), 8.10-8.33 (m, 1H), 7.98-7.99 (m, 1H), 7.81-7.89 (m, 3H), 7.25-7.32 (m, 4H), 6.55-6.63 (m, 1H), 6.44-6.47 (m, 1H), 5.55-5.59 (m, 1H), 2.98-3.04 (m, 2H), 2.50-2.51 (m, 1H), 2.03-2.06 (m, 1H). ¹⁹F-NMR (400 MHz, DMSO-d₆) δ (ppm): −174.05.

### Intermediate 76-2: (S)-3-(3-(1-amino-2,3-dihydro-1H-inden-5-yl)-5-(4-fluoro-1H-pyrazol-1-yl)-3H-imidazo[4,5-b]pyridin-2-yl)pyridin-2-amine

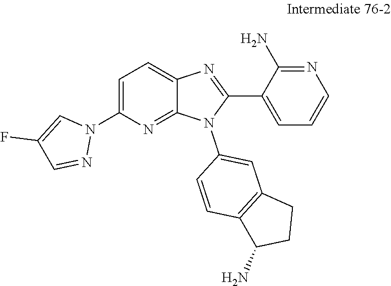 [SMILES: Nc1ncccc1-c1nc2ccc(-n3cc(F)cn3)nc2n1-c1ccc2c(c1)CC[C@@H]2N]

Intermediate 76-2 was prepared in a manner analogous to Intermediate 13-2 (starting from Step 2) using Intermediate 76-1 in place of the nitro starting material. MS (ESI) calculated for C₂₃H₁₉FN₈: 426.17 m/z, found 427.10 [M+H]⁺.

### Intermediate 76-1: (S)—N-(5-((6-(4-fluoro-1H-pyrazol-1-yl)-3-nitropyridin-2-yl)amino)-2,3-dihydro-1H-inden-1-yl)acetamide

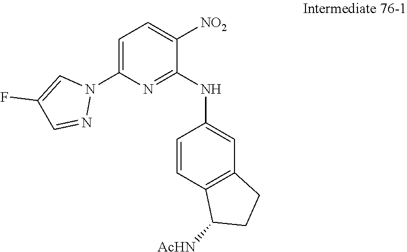 [SMILES: CC(=O)N[C@H]1CCc2cc(Nc3nc(-n4cc(F)cn4)ccc3[N+](=O)[O-])ccc21]

### Synthetic Route:

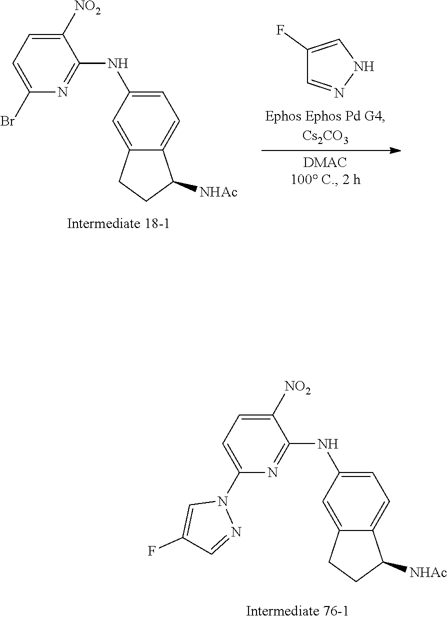 [SMILES: CC(=O)N[C@H]1CCc2cc(Nc3nc(Br)ccc3[N+](=O)[O-])ccc21 ; Fc1cn[nH]c1 ; CC(=O)N[C@H]1CCc2cc(Nc3nc(-n4cc(F)cn4)ccc3[N+](=O)[O-])ccc21]

### Step 1: Synthesis of N-[(1S)-5-{[6-(4-fluoropyrazol-1-yl)-3-nitropyridin-2-yl]amino}-2,3-dihydro-1H-inden-1-yl]acetamide (Intermediate 76-1)

To a solution of N-[(1S)-5-[(6-bromo-3-nitropyridin-2-yl)amino]-2,3-dihydro-1H-inden-1-yl]acetamide (Intermediate 18-1) (1.00 g, 2.56 mmol, 1 equiv) in 1,4-dioxane (10 mL) was added 4-fluoro-1H-pyrazole (0.33 g, 3.8 mmol, 1.5 equiv), EPhos (0.21 g, 0.38 mmol, 0.15 equiv), EPhos Pd G4 (0.23 g, 0.26 mmol, 0.1 equiv) and cesium carbonate (1.67 g, 5.11 mmol, 2 equiv). The resulting mixture was stirred at 100° C. under nitrogen atmosphere for 2 h. The reaction mixture was then cooled to room temperature and concentrated in vacuo. The residue was purified by reverse-phase flash column chromatography on C18 silica gel using a 20-95% gradient of acetonitrile in water (+0.05% ammonium bicarbonate) with a 10 minute hold at 70% acetonitrile to afford N-[(1S)-5-{[6-(4-fluoropyrazol-1-yl)-3-nitropyridin-2-yl]amino}-2,3-dihydro-1H-inden-1-yl]acetamide (Intermediate 76-1) (400 mg, 36%) as a yellow solid. MS (ESI) calculated for C₁₉H₁₇FN₆O₃: 396.13 m/z, found 397.15 [M+H]⁺.

### Example 77: (S)—N-(5-(2-(2-aminopyridin-3-yl)-5-(4-methyl-1H-pyrazol-1-yl)-3H imidazo[4,5-b]pyridin-3-yl)-2,3-dihydro-1H-inden-1-yl)-3-formyl-4-hydroxybenzamide

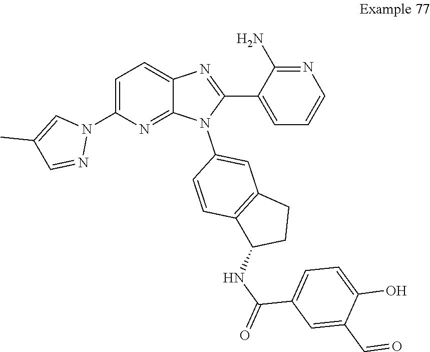 [SMILES: Cc1cnn(-c2ccc3nc(-c4cccnc4N)n(-c4ccc5c(c4)CC[C@@H]5NC(=O)c4ccc(O)c(C=O)c4)c3n2)c1]

Example 77 was prepared in a manner analogous to Example 12 using Intermediate 77-2 in place of Intermediate 1-3, HATU in place of PyBOP and Intermediate 57-2 in place of Intermediate 12-1. MS (ESI) calculated for C₃₂H₂₆N₈O₃: 570.21 m/z, found 571.30 [M+H]⁺. ¹H NMR (400 MHz, DMSO-d₆) δ (ppm): 10.29 (s, 1H), 8.23-8.42 (m, 2H), 8.04-8.18 (m, 2H), 8.00-8.04 (m, 1H), 7.88-8.00 (m, 1H), 7.55-7.70 (m, 1H), 7.45-7.55 (m, 1H), 7.25-7.45 (m, 3H), 7.00-7.16 (m, 1H), 6.52-6.70 (m, 1H), 5.50-5.72 (m, 1H), 3.00-3.12 (m, 1H), 2.85-3.00 (m, 1H), 2.40-2.50 (m, 1H), 2.00-2.21 (m, 4H).

### Intermediate 77-2: (S)-3-(3-(1-amino-2,3-dihydro-1H-inden-5-yl)-5-(4-methyl-1H-pyrazol-1-yl)-3H-imidazo[4,5-b]pyridin-2-yl)pyridin-2-amine

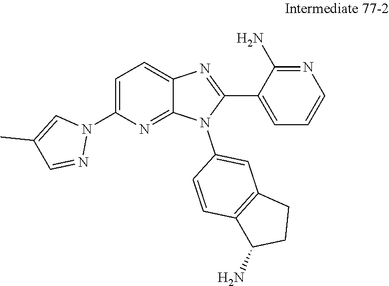 [SMILES: Cc1cnn(-c2ccc3nc(-c4cccnc4N)n(-c4ccc5c(c4)CC[C@@H]5N)c3n2)c1]

Intermediate 77-2 was prepared in a manner analogous to Intermediate 13-2 (starting from Step 2) using Intermediate 77-1 in place of the nitro starting material. MS (ESI) calculated for C₂₄H₂₂N₈: 422.20 m/z, found 423.20 [M+H]⁺.

### Intermediate 77-1: (S)—N-(5-((6-(4-methyl-1H-pyrazol-1-yl)-3-nitropyridin-2-yl)amino)-2,3-dihydro-1H-inden-1-yl)acetamide

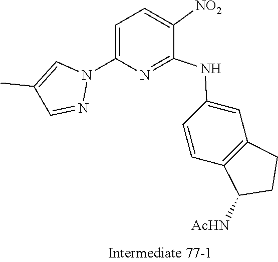 [SMILES: CC(=O)N[C@H]1CCc2cc(Nc3nc(-n4cc(C)cn4)ccc3[N+](=O)[O-])ccc21]

Intermediate 77-1 was prepared in a manner analogous to Intermediate 76-1 using 4-methyl-1H-pyrazole in place of 4-fluoro-1H-pyrazole. MS (ESI) calculated for C₂₀H₂₀N₆O₃: 392.16 m/z, found 393.15 [M+H]⁺.

### Example 78: N-[(1S)-5-[2-(2-aminopyridin-3-yl)-5-(4-chloropyrazol-1-yl)imidazo[4,5-b]pyridin-3-yl]-2,3-dihydro-1H-inden-1-yl]-3-formyl-4-hydroxybenzamide

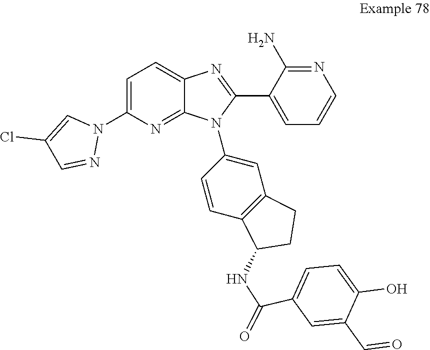 [SMILES: Nc1ncccc1-c1nc2ccc(-n3cc(Cl)cn3)nc2n1-c1ccc2c(c1)CC[C@@H]2NC(=O)c1ccc(O)c(C=O)c1]

Example 78 was prepared in a manner analogous to Example 12 using Intermediate 78-1 in place of Intermediate 1-3 and Intermediate 57-2 in place of Intermediate 12-1. MS (ESI) calculated for C₃₁H₂₃ClN₈O₃: 590.15 m/z, found 591.15 [M+H]⁺. ¹H NMR (400 MHz, DMSO-d₆) δ (ppm): 10.28 (s, 1H), 8.94-8.96 (m, 1H), 8.35-8.38 (m, 1H), 8.29-8.30 (m, 1H), 8.09-8.10 (m, 1H), 8.07-8.08 (m, 1H), 8.00-8.01 (m, 1H), 7.89-7.93 (m, 2H), 7.26-7.36 (m, 4H), 7.05-7.07 (m, 1H), 6.45-6.47 (m, 1H), 5.59-5.65 (m, 1H), 2.85-3.06 (m, 2H), 2.50-2.51 (m, 1H), 2.05-2.10 (m, 1H).

### Intermediate 78-1: (S)-3-(3-(1-amino-2,3-dihydro-1H-inden-5-yl)-5-(4-chloro-1H-pyrazol-1-yl)-3H-imidazo[4,5-b]pyridin-2-yl)pyridin-2-amine

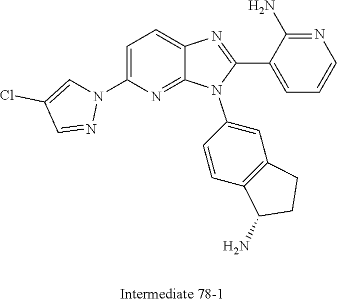 [SMILES: Nc1ncccc1-c1nc2ccc(-n3cc(Cl)cn3)nc2n1-c1ccc2c(c1)CC[C@@H]2N]

Intermediate 78-1 was prepared in a manner analogous to Intermediate 13-2 using Intermediate 18-1 in place of Intermediate 13-1, 4-chloro-1H-pyrazole in place of 6-methyl-3-nitropyridin-2-amine and tris(dibenzylideneacetone)dipalladium(0) in place of palladium(II) acetate. MS (ESI) calculated for C₂₃H₁₉ClN₈: 442.14 m/z, found 443.15 [M+H]⁺.

### Example 79: (S)—N-(5-(2-(2-aminopyridin-3-yl)-5-cyano-3H-imidazo[4,5-b]pyridin-3-yl)-2,3-dihydro-1H-inden-1-yl)-3-formyl-4-hydroxybenzamide

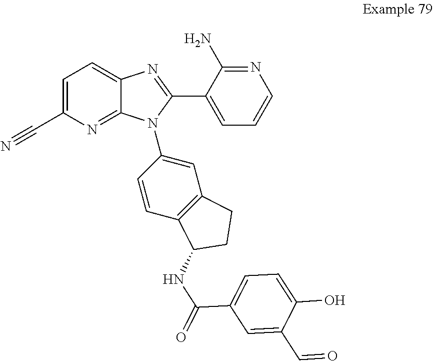 [SMILES: N#Cc1ccc2nc(-c3cccnc3N)n(-c3ccc4c(c3)CC[C@@H]4NC(=O)c3ccc(O)c(C=O)c3)c2n1]

Example 79 was prepared in a manner analogous to Example 12 using Intermediate 79-3 in place of Intermediate 1-3 and Intermediate 57-2 in place of Intermediate 12-1. MS (ESI) calculated for C₂₉H₂₁N₇O₃: 515.17 m/z, found 516.25 [M+H]⁺. ¹H NMR (400 MHz, DMSO-d₆) δ (ppm): 10.23 (s, 1H), 8.37-8.39 (m, 1H), 8.18 (s, 1H), 7.92-8.03 (m, 3H), 7.32-7.38 (m, 3H), 7.24-7.26 (m, 1H), 6.83 (s, 1H), 6.42-6.48 (m, 1H), 5.58-5.61 (m, 1H), 2.99-3.01 (m, 1H), 2.86-2.90 (m, 1H), 2.50-2.51 (m, 1H), 2.03-2.08 (m, 1H).

### Intermediate 79-3: (S)-3-(1-amino-2,3-dihydro-1H-inden-5-yl)-2-(2-aminopyridin-3-yl)-3H-imidazo[4,5-b]pyridine-5-carbonitrile

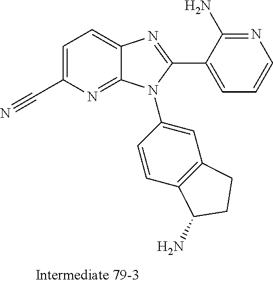 [SMILES: N#Cc1ccc2nc(-c3cccnc3N)n(-c3ccc4c(c3)CC[C@@H]4N)c2n1]

### Synthetic Route:

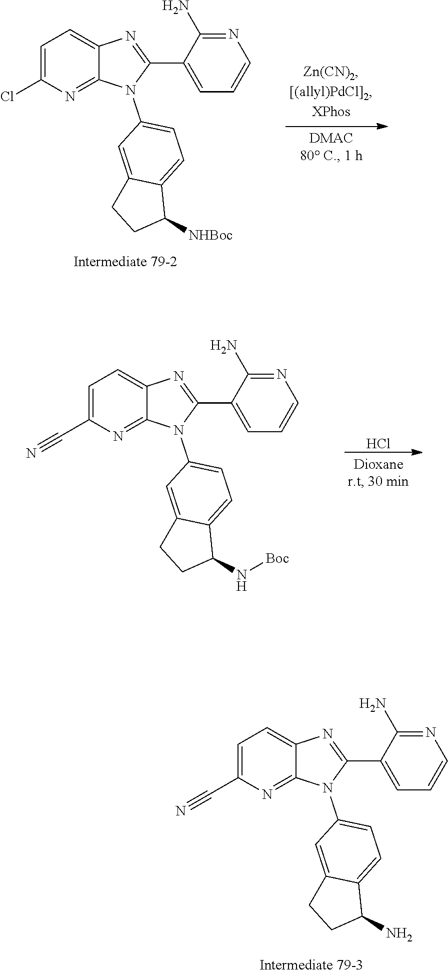 [SMILES: CC(C)(C)OC(=O)N[C@H]1CCc2cc(-n3c(-c4cccnc4N)nc4ccc(Cl)nc43)ccc21 ; CC(C)(C)OC(=O)N[C@H]1CCc2cc(-n3c(-c4cccnc4N)nc4ccc(C#N)nc43)ccc21 ; Cl ; N#Cc1ccc2nc(-c3cccnc3N)n(-c3ccc4c(c3)CC[C@@H]4N)c2n1]

### Step 1: Synthesis of tert-butyl (S)-(5-(2-(2-aminopyridin-3-yl)-5-cyano-3H-imidazo[4,5-b]pyridin-3-yl)-2,3-dihydro-1H-inden-1-yl)carbamate

A mixture of tert-butyl (S)-(5-(2-(2-aminopyridin-3-yl)-5-chloro-3H-imidazo[4,5-b]pyridin-3-yl)-2,3-dihydro-1H-inden-1-yl)carbamate (Intermediate 79-2) (200 mg, 0.419 mmol, 1 equiv), zinc (II) cyanide (123 mg, 1.05 mmol, 2.5 equiv), XPhos (40 mg, 0.084 mmol, 0.2 equiv), dimethyl acetamide (1.5 mL) and allylpalladium(II) chloride dimer (15 mg, 0.042 mmol, 0.1 equiv) were stirred at 80° C. under nitrogen atmosphere for 1 h. The resulting mixture was purified by reverse-phase flash column chromatography on C18 silica gel using a 0-85% gradient of acetonitrile in water (+0.05% ammonium bicarbonate) to afford tert-butyl (S)-(5-(2-(2-aminopyridin-3-yl)-5-cyano-3H-imidazo[4,5-b]pyridin-3-yl)-2,3-dihydro-1H-inden-1-yl)carbamate (125 mg, 32%) as a yellow solid. MS (ESI) calculated for C₂₆H₂₅N₇O₂: 467.21 m/z, found 468.15 [M+H]⁺.

### Step 2: Synthesis of (S)-3-(1-amino-2,3-dihydro-1H-inden-5-yl)-2-(2-aminopyridin-3-yl)-3H-imidazo[4,5-b]pyridine-5-carbonitrile (Intermediate 79-3)

tert-Butyl (S)-(5-(2-(2-aminopyridin-3-yl)-5-cyano-3H-imidazo[4,5-b]pyridin-3-yl)-2,3-dihydro-1H-inden-1-yl)carbamate (125 mg, 0.135 mmol, 1 equiv) was dissolved in 4N hydrochloric acid in dioxane (5 mL) and the resulting mixture was stirred at room temperature for 30 min. The mixture was concentrated in vacuo to afford crude (S)-3-(1-amino-2,3-dihydro-1H-inden-5-yl)-2-(2-aminopyridin-3-yl)-3H-imidazo[4,5-b]pyridine-5-carbonitrile (Intermediate 79-3) as a yellow solid, which was used without purification in subsequent transformations. MS (ESI) calculated for C₂₁H₁₇N₇: 367.15 m/z, found 368.15 [M+H]⁺.

### Intermediate 79-2: tert-butyl (S)-(5-(2-(2-aminopyridin-3-yl)-5-chloro-3H-imidazo[4,5-b]pyridin-3-yl)-2,3-dihydro-1H-inden-1-yl)carbamate

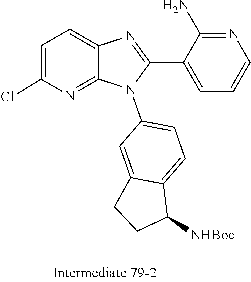 [SMILES: CC(C)(C)OC(=O)N[C@H]1CCc2cc(-n3c(-c4cccnc4N)nc4ccc(Cl)nc43)ccc21]

### Synthetic Route:

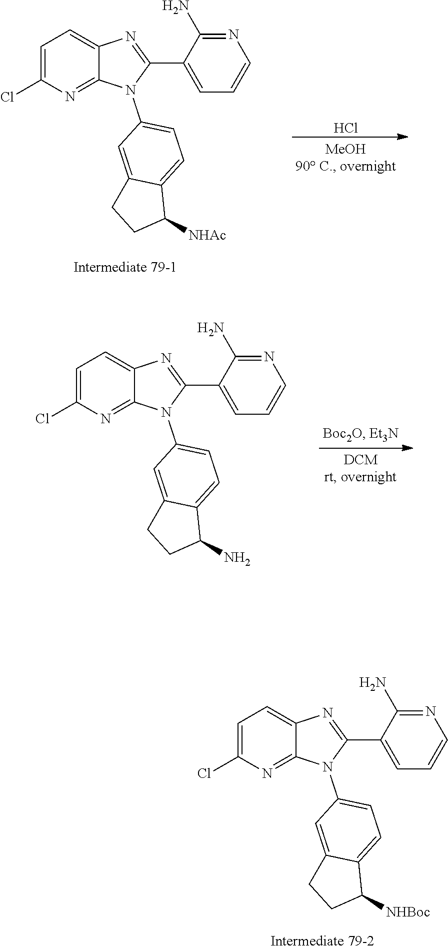 [SMILES: CC(=O)N[C@H]1CCc2cc(-n3c(-c4cccnc4N)nc4ccc(Cl)nc43)ccc21 ; Cl ; Nc1ncccc1-c1nc2ccc(Cl)nc2n1-c1ccc2c(c1)CC[C@@H]2N ; CC(C)(C)OC(=O)N[C@H]1CCc2cc(-n3c(-c4cccnc4N)nc4ccc(Cl)nc43)ccc21]

### Step 1: Synthesis of (S)-3-(3-(1-amino-2,3-dihydro-1H-inden-5-yl)-5-chloro-3H-imidazo[4,5-b]pyridin-2-yl)pyridin-2-amine

(S)—N-(5-(2-(2-aminopyridin-3-yl)-5-chloro-3H-imidazo[4,5-b]pyridin-3-yl)-2,3-dihydro-1H-inden-1-yl)acetamide (Intermediate 79-1) (200 mg, 0.478 mmol, 1 equiv) was dissolved in methanol (10 mL) and hydrochloric acid (10 mL, conc.) was added. The resulting mixture was stirred overnight at 90° C. The mixture was then cooled to room temperature and concentrated in vacuo to afford crude (S)-3-(3-(1-amino-2,3-dihydro-1H-inden-5-yl)-5-chloro-3H-imidazo[4,5-b]pyridin-2-yl)pyridin-2-amine as a yellow solid, which was used directly in the next step without further purification. MS (ESI) calculated for C₂₀H₁₇ClN₆: 376.12 m/z, found 377.15 [M+H]⁺.

### Step 2: Synthesis of tert-butyl (S)-(5-(2-(2-aminopyridin-3-yl)-5-chloro-3H-imidazo[4,5-b]pyridin-3-yl)-2,3-dihydro-1H-inden-1-yl)carbamate (Intermediate 79-2)

Crude (S)-3-(3-(1-amino-2,3-dihydro-1H-inden-5-yl)-5-chloro-3H-imidazo[4,5-b]pyridin-2-yl)pyridin-2-amine was dissolved in dichloromethane (10 mL). di-tert-Butyl dicarbonate (148 mg, 0.678 mmol, 1.5 equiv) was added followed by triethylamine (137 mg, 1.36 mmol, 3 equiv) and the resulting mixture was stirred at room temperature for 2 h. The reaction was quenched by the addition of water (10 mL). The resulting mixture was extracted with ethyl acetate (3×20 mL). The organic layers were combined, dried over anhydrous sodium sulfate, filtered, and concentrated in vacuo. The residue obtained was purified by silica gel column chromatography eluting with dichloromethane/methanol (10:1) to afford tert-butyl (S)-(5-(2-(2-aminopyridin-3-yl)-5-chloro-3H-imidazo[4,5-b]pyridin-3-yl)-2,3-dihydro-1H-inden-1-yl)carbamate (Intermediate 79-2) (180 mg, 80% over two steps) as a yellow solid. MS (ESI) calculated for C₂₅H₂₅ClN₆O₂: 476.17 m/z, found 477.20 [M+H]⁺.

### Intermediate 79-1: (S)—N-(5-(2-(2-aminopyridin-3-yl)-5-chloro-3H-imidazo[4,5-b]pyridin-3-yl)-2,3-dihydro-1H-inden-1-yl)acetamide

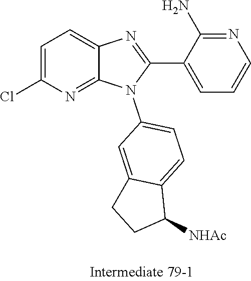 [SMILES: CC(=O)N[C@H]1CCc2cc(-n3c(-c4cccnc4N)nc4ccc(Cl)nc43)ccc21]

### Synthetic Route:

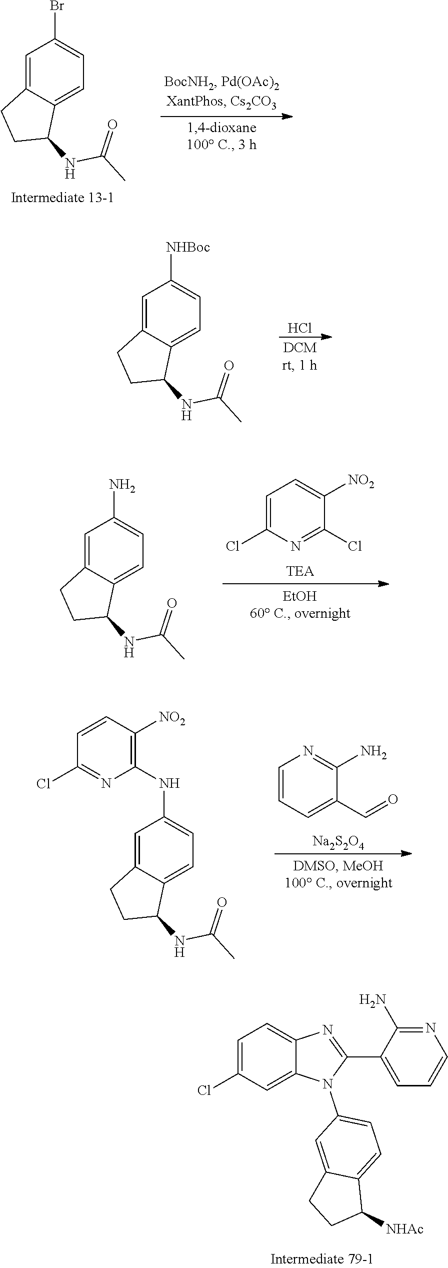

### Step 1: Synthesis of tert-butyl (S)-(1-acetamido-2,3-dihydro-1H-inden-5-yl)carbamate

To a mixture of N-[(1S)-5-bromo-2,3-dihydro-1H-inden-1-yl]acetamide (Intermediate 13-1) (40 g, 157 mmol, 1 equiv), tert-butyl carbamate (27.66 g, 236 mmol, 1.5 equiv), XantPhos (9.11 g, 15.7 mmol, 10 mol %), palladium (II) acetate (3.54 g, 15.7 mmol, 10 mol %), and cesium carbonate (154 g, 472 mmol, 10 mol %) was added 1,4-dioxane (300 mL) under nitrogen atmosphere. The resulting mixture was stirred for 3 h at 100° C. The reaction mixture was quenched by addition of water (50 mL) and extracted with ethyl acetate (3×50 mL). The combined organic layers were dried over anhydrous sodium sulfate and concentrated under reduced pressure. The residue was purified by silica gel column chromatography using an eluent of petroleum ether/dichloromethane/methanol (70:27:3) to afford tert-butyl N-[(1S)-1-acetamido-2,3-dihydro-1H-inden-5-yl]carbamate (43.1 g, 48%). MS (ESI) calculated for C₁₆H₂₂N₂O₃: 290.16 m/z, found 289.05 [M−H]⁻.

### Step 2: Synthesis of (S)—N-(5-amino-2,3-dihydro-1H-inden-1-yl)acetamide

To a stirred solution of tert-butyl N-[(1S)-1-acetamido-2,3-dihydro-1H-inden-5-yl]carbamate (43.1 g, 148 mmol, 1 equiv) in dichloromethane (180 mL) was added 4N hydrochloric acid in 1,4-dioxane (185 mL, 742 mmol, 5 equiv). The reaction mixture was stirred for 1 h at room temperature. The reaction mixture was concentrated in vacuo and re-crystallized from ethyl acetate to afford N-[(1S)-5-amino-2,3-dihydro-1H-inden-1-yl]acetamide (hydrochloride salt) (23 g, 81%) as a white solid. MS (ESI) calculated for C₁₁H₁₄N₂O: 190.11 m/z, found 191.15 [M+H]⁺.

### Step 3: Synthesis of N-[(1S)-5-[(6-chloro-3-nitropyridin-2-yl)amino]-2,3-dihydro-1H-inden-1-yl]acetamide

To a solution of N-[(1S)-5-amino-2,3-dihydro-1H-inden-1-yl]acetamide (100 g, 526 mmol, 1 equiv) in ethanol (2 L) was added triethylamine (160 g, 1.58 mol, 3 equiv) and 2,6-dichloro-3-nitropyridine (122 g, 631 mmol, 1.2 equiv). The resulting mixture was stirred at 60° C. overnight. The mixture was then cooled to room temperature and quenched with water. The resulting precipitate was collected by filtration and rinsed with ethanol/water to afford N-[(1S)-5-[(6-chloro-3-nitropyridin-2-yl)amino]-2,3-dihydro-1H-inden-1-yl]acetamide (100 g, 49%) as a red solid. MS (ESI) calculated for C₁₆H₁₅ClN₄O₃: 346.08 m/z, found 345.00 [M−H]⁻.

### Step 4: Synthesis of N-[(1S)-5-[2-(2-aminopyridin-3-yl)-5-chloroimidazo[4,5-b]pyridin-3-yl]-2,3-dihydro-1H-inden-1-yl]acetamide (Intermediate 79-1)

To a solution of N-[(1S)-5-[(6-chloro-3-nitropyridin-2-yl)amino]-2,3-dihydro-1H-inden-1-yl]acetamide (100 g, 288 mmol, 1 equiv) in dimethyl sulfoxide (1.8 L) and methanol (300 mL) was added 2-aminopyridine-3-carbaldehyde (38.74 g, 317.2 mmol, 1.1 equiv) and sodium dithionite (110 g, 634 mmol, 2.2 equiv). The resulting mixture was stirred at 100° C. overnight. Water was added and the precipitated solids were collected by filtration, rinsing with water. The solid collected was purified by silica gel column chromatography eluting with dichloromethane/methanol (10:1) to afford N-[(1S)-5-[2-(2-aminopyridin-3-yl)-5-chloroimidazo[4,5-b]pyridin-3-yl]-2,3-dihydro-1H-inden-1-yl]acetamide (Intermediate 79-1) (43.2 g, 31%) as a yellow solid. MS (ESI) calculated for C₂₂H₁₉ClN₆O: 418.13 m/z, found 419.10 [M+H]⁺.

### Example 80: N-[(1S)-5-[2-(2-aminopyridin-3-yl)-5-(6-methoxypyrazin-2-yl)imidazo[4,5-b]pyridin-3-yl]-2,3-dihydro-1H-inden-1-yl]-3-formyl-4-hydroxybenzamide

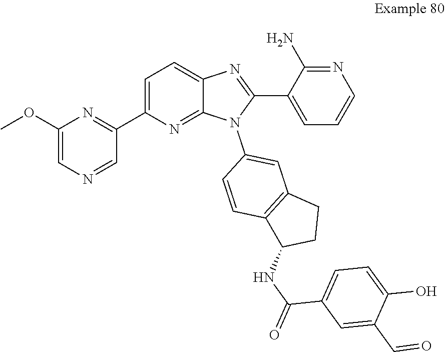 [SMILES: COc1cncc(-c2ccc3nc(-c4cccnc4N)n(-c4ccc5c(c4)CC[C@@H]5NC(=O)c4ccc(O)c(C=O)c4)c3n2)n1]

Example 80 was prepared in a manner analogous to Example 19 using Intermediate 79-2 in place of Intermediate 18-2, Intermediate 80-1 in place of 3-(4-methylpiperazin-1-yl)phenylboronic acid, 4N hydrochloric acid in 1,4-dioxane at room temperature for 1 h instead of hydrochloric acid in methanol at 90° C. overnight, PyBOP in place of HATU and Intermediate 57-2 in place of Intermediate 5-1. MS (ESI) calculated for C₃₃H₂₆N₈O₄: 598.21 m/z, found 599.25 [M+H]⁺. ¹H NMR (400 MHz, DMSO-d₆) δ (ppm): 10.28 (s, 1H), 8.88-8.90 (m, 1H), 8.06-8.42 (m, 4H), 8.02-8.07 (m, 2H), 7.28-7.42 (m, 4H), 7.00-7.02 (m, 1H), 6.45-6.48 (m, 1H), 5.63-5.66 (m, 1H), 4.05 (s, 3H), 2.92-3.08 (m, 1H), 2.88-2.90 (m, 1H), 2.47-2.49 (m, 1H), 2.06-2.09 (m, 1H).

### Intermediate 80-1: (6-methoxypyrazin-2-yl)boronic acid

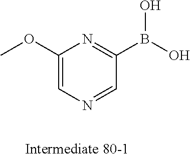

Intermediate 80-1 was prepared in a manner analogous to Intermediate 60-1 using 2-bromo-6-methoxypyrazine in place of 2-bromo-6-methylpyrazine. MS (ESI) calculated for C₅H₇BN₂O₃: 154.05 m/z, found 155.10 [M+H]⁺.

### Example 81: N-[(1S)-5-[2-(2-aminopyridin-3-yl)-5-(pyrazol-1-yl)imidazo[4,5-b]pyridin-3-yl]-2,3-dihydro-1H-inden-1-yl]-3-fluoro-5-formyl-4-hydroxybenzamide

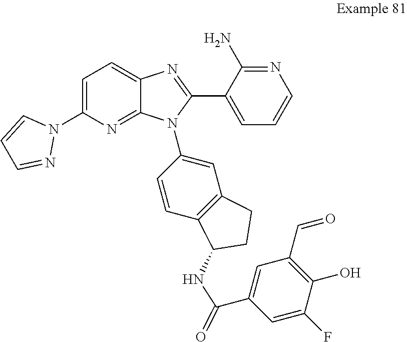 [SMILES: Nc1ncccc1-c1nc2ccc(-n3cccn3)nc2n1-c1ccc2c(c1)CC[C@@H]2NC(=O)c1cc(F)c(O)c(C=O)c1]

Example 81 was prepared in a manner analogous to Example 12 using Intermediate 33-1 in place of Intermediate 1-3 and Intermediate 81-1 in place of Intermediate 12-1. MS (ESI) calculated for C₃₁H₂₃FN₈O₃: 574.19 m/z, found 575.15 [M+H]⁺. ¹H NMR (400 MHz, DMSO-d₆) δ (ppm): 10.28 (s, 1H), 8.42-8.44 (m, 1H), 8.37-8.38 (m, 1H), 8.06-8.07 (m, 1H), 8.00-8.02 (m, 1H), 7.89-7.82 (m, 1H), 7.56-7.77 (m, 1H), 7.52-7.54 (m, 1H), 7.41-7.47 (m, 2H), 7.36-7.37 (m, 1H), 7.11-7.14 (m, 1H), 6.79-6.83 (m, 1H), 6.55-6.56 (m, 1H), 5.54-5.58 (m, 1H), 2.87-3.04 (m, 2H), 2.56-2.58 (m, 1H), 1.99-2.05 (m, 1H). ¹⁹F NMR (376 MHz, DMSO-d₆) δ (ppm): −137.15.

### Intermediate 81-1: 3-(1,3-dioxolan-2-yl)-5-fluoro-4-((4-methoxybenzyl)oxy)benzoic acid

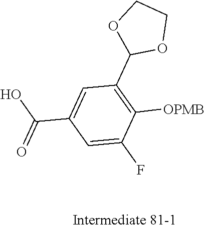

Intermediate 81-1 was prepared in a manner analogous to Intermediate 5-1 (via Intermediate 2-1) starting from 5-bromo-3-fluoro-2-hydroxybenzaldehyde instead of 5-bromo-2-hydroxybenzaldehyde. MS (ESI) calculated for C₁₈H₁₇FO₆: 348.10 m/z, found 347.10 [M−H]⁻.

### Example 82: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2,3-difluoro-5-formyl-4-hydroxybenzamide

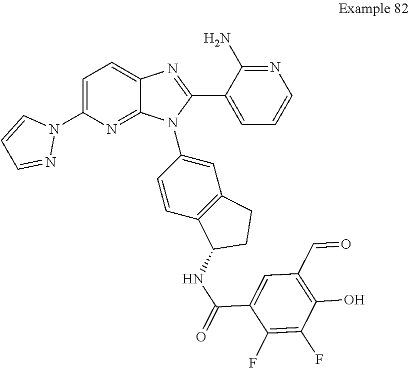 [SMILES: Nc1ncccc1-c1nc2ccc(-n3cccn3)nc2n1-c1ccc2c(c1)CC[C@@H]2NC(=O)c1cc(C=O)c(O)c(F)c1F]

Example 82 was prepared in a manner analogous to Example 22 using Intermediate 33-1 in place of Intermediate 1-3. MS (ESI) calculated for C₃₁H₂₂F₂N₈O₃: 592.18 m/z, found 593.25 [M+H]⁺. ¹H NMR (400 MHz, DMSO-d₆) δ (ppm): 10.20 (s, 1H), 8.41-8.43 (m, 1H), 8.36-8.37 (m, 1H), 8.01-8.06 (m, 2H), 7.83-7.99 (m, 2H), 7.72-7.82 (m, 1H), 7.41-7.45 (m, 1H), 7.34-7.36 (m, 2H), 6.76-6.79 (m, 1H), 6.56-6.57 (m, 1H), 5.52-5.57 (m, 1H), 2.88-3.03 (m, 2H), 2.54-2.56 (m, 1H), 2.00-2.06 (m, 1H). ¹⁹F NMR (376 MHz, DMSO-d₆) δ (ppm): −128.36, −158.15.

### Example 83: N-[(1S)-5-[2-(2-aminopyridin-3-yl)-5-(4-methoxypyrazol-1-yl)imidazo[4,5-b]pyridin-3-yl]-2,3-dihydro-1H-inden-1-yl]-3-formyl-4-hydroxybenzamide

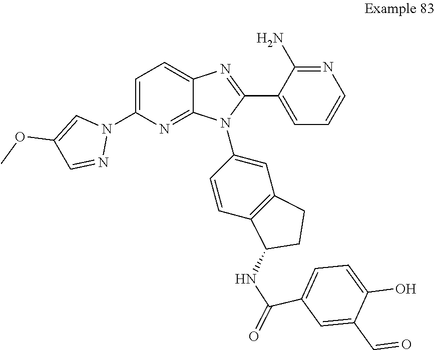 [SMILES: COc1cnn(-c2ccc3nc(-c4cccnc4N)n(-c4ccc5c(c4)CC[C@@H]5NC(=O)c4ccc(O)c(C=O)c4)c3n2)c1]

Example 83 was prepared in a manner analogous to Example 12 using Intermediate 83-2 in place of Intermediate 1-3 and Intermediate 5-1 in place of Intermediate 12-1. MS (ESI) calculated for C₃₂H₂₆N₈O₄: 586.21 m/z, found 587.15 [M+H]⁺. ¹H NMR (400 MHz, DMSO-d₆) δ (ppm): 10.10 (s, 1H), 8.18-8.20 (m, 1H), 7.93-8.10 (m, 1H), 7.91-7.92 (m, 3H), 7.85-7.87 (m, 1H), 7.77-7.79 (m, 1H), 7.14-7.27 (m, 4H), 6.79-6.80 (m, 1H), 6.43-6.46 (m, 1H), 5.49-5.53 (m, 1H), 3.65-3.69 (m, 3H), 2.79-2.99 (m, 2H), 2.46-2.50 (m, 1H), 2.00-2.05 (m, 1H).

### Intermediate 83-2: (S)-3-(3-(1-amino-2,3-dihydro-1H-inden-5-yl)-5-(4-methoxy-1H-pyrazol-1-yl)-3H-imidazo[4,5-b]pyridin-2-yl)pyridin-2-amine

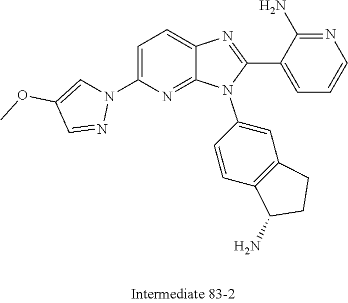 [SMILES: COc1cnn(-c2ccc3nc(-c4cccnc4N)n(-c4ccc5c(c4)CC[C@@H]5N)c3n2)c1]

Intermediate 83-2 was prepared in a manner analogous to Intermediate 69-1 (starting from Step 2) using Intermediate 83-1 in place of the nitro starting material. MS (ESI) calculated for C₂₄H₂₂N₈O: 438.19 m/z, found 439.15 [M+H]⁺.

### Intermediate 83-1: (S)—N-(5-((6-(4-methoxy-1H-pyrazol-1-yl)-3-nitropyridin-2-yl)amino)-2,3-dihydro-1H-inden-1-yl)acetamide

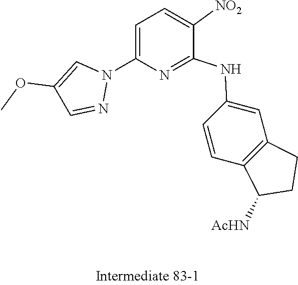 [SMILES: COc1cnn(-c2ccc([N+](=O)[O-])c(Nc3ccc4c(c3)CC[C@@H]4NC(C)=O)n2)c1]

Intermediate 83-1 was prepared in a manner analogous to Intermediate 76-1 using 4-methoxypyrazole in place of 4-fluoropyrazole. MS (ESI) calculated for C₂₀H₂₀N₆O₄: 408.15 m/z, found 409.15 [M+H]⁺.

### Example 84: N-[(1S)-5-[2-(2-aminopyridin-3-yl)-5-(4-formylpyrazol-1-yl)imidazo[4,5-b]pyridin-3-yl]-2,3-dihydro-1H-inden-1-yl]-3-formyl-4-hydroxybenzamide

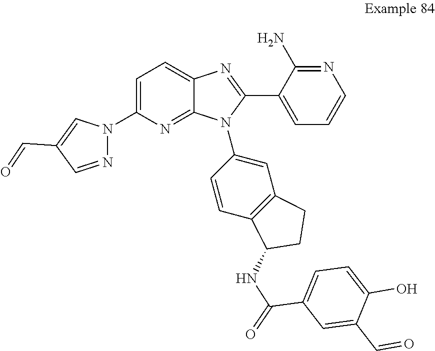 [SMILES: Nc1ncccc1-c1nc2ccc(-n3cc(C=O)cn3)nc2n1-c1ccc2c(c1)CC[C@@H]2NC(=O)c1ccc(O)c(C=O)c1]

Example 84 was prepared in a manner analogous to Example 12 using Intermediate 84-1 in place of Intermediate 1-3 and Intermediate 57-2 in place of Intermediate 12-1. MS (ESI) calculated for C₃₂H₂₄N₈O₄: 584.19 m/z, found 585.25 [M+H]⁺. ¹H NMR (400 MHz, DMSO-d₆) δ (ppm): 10.18 (s, 1H), 9.85 (s, 1H), 8.97 (s, 1H), 8.35-8.37 (m, 1H), 8.17-8.25 (m, 1H), 7.98-7.99 (m, 1H), 7.94-7.98 (m, 3H), 7.26-7.34 (m, 4H), 6.91-6.93 (m, 1H), 6.47-6.51 (m, 1H), 5.53-5.58 (m, 1H), 2.98-3.00 (m, 1H), 2.87-2.88 (m, 1H), 2.50-2.51 (m, 1H), 2.02-2.08 (m, 1H).

### Intermediate 84-1: (S)-1-(3-(1-amino-2,3-dihydro-1H-inden-5-yl)-2-(2-aminopyridin-3-yl)-3H-imidazo[4,5-b]pyridin-5-yl)-1H-pyrazole-4-carbaldehyde

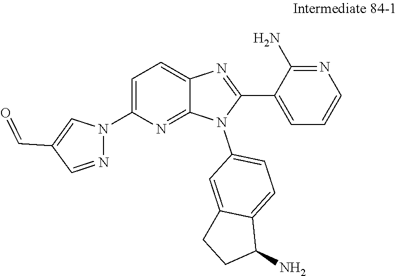 [SMILES: Nc1ncccc1-c1nc2ccc(-n3cc(C=O)cn3)nc2n1-c1ccc2c(c1)CC[C@@H]2N]

### Synthetic Route:

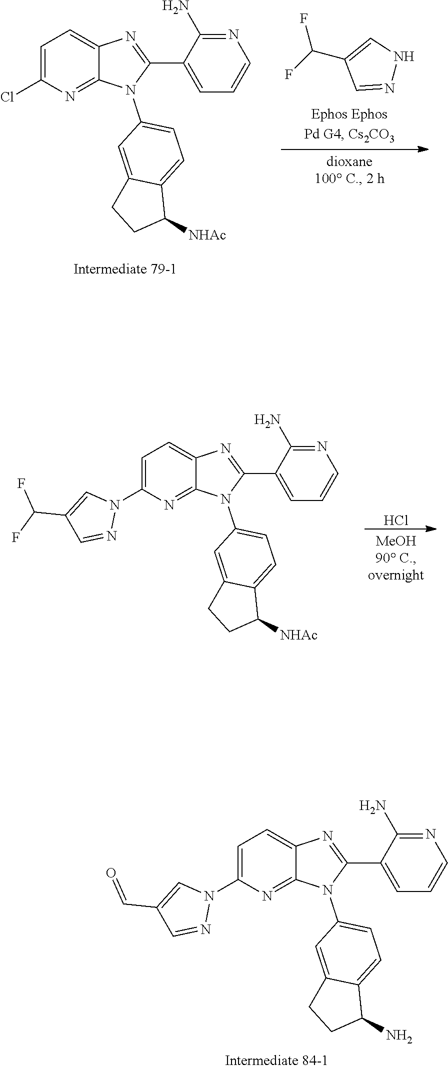

### Step 1: Synthesis of N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]acetamide

To a solution of N-[(1S)-5-[2-(2-aminopyridin-3-yl)-5-chloroimidazo[4,5-b]pyridin-3-yl]-2,3-dihydro-1H-inden-1-yl]acetamide (Intermediate 79-1) (1.00 g, 2.39 mmol, 1 equiv) in 1,4-dioxane (10 mL, 113.500 mmol) were added 4-(difluoromethyl)-1H-pyrazole (0.42 g, 3.6 mmol, 1.5 equiv), EPhos (0.19 g, 0.36 mmol, 0.15 equiv), EPhos Pd G4 (0.22 g, 0.24 mmol, 0.1 equiv) and cesium carbonate (1.56 g, 4.78 mmol, 2 equiv). The resulting mixture was stirred under nitrogen atmosphere 100° C. for 2 h. The mixture was concentrated in vacuo and the residue was purified by reverse-phase flash column chromatography on C18 silica gel using a 20-95% gradient of acetonitrile in water (+0.05% ammonium bicarbonate) with a 10-minute hold at 70% acetonitrile to afford N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]acetamide (160 mg, 12%) as a yellow solid. MS (ESI) calculated for C₂₆H₂₂F₂N₈O: 500.19 m/z, found 501.15 [M+H]⁺.

### Step 2: Synthesis of 1-{3-[(1S)-1-amino-2,3-dihydro-1H-inden-5-yl]-2-(2-aminopyridin-3-yl)imidazo[4,5-b]pyridin-5-yl}pyrazole-4-carbaldehyde (Intermediate 84-1)

A solution of N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]acetamide (160 mg, 0.320 mmol, 1 equiv) in hydrochloric acid (15 mL, conc.) and methanol (15 mL) was stirred at 90° C. overnight. The solvent was removed by distillation under vacuum to afford crude 1-{3-[(1S)-1-amino-2,3-dihydro-1H-inden-5-yl]-2-(2-aminopyridin-3-yl)imidazo[4,5-b]pyridin-5-yl}pyrazole-4-carbaldehyde (Intermediate 84-1) (130 mg, 93%) as a brown solid, which was used without further purification in subsequent transformations. MS (ESI) calculated for C₂₄H₂₀N₈O: 436.18 m/z, found 437.20 [M+H]⁺.

### Example 85: N-[(1S)-5-[2-(2-aminopyridin-3-yl)-5-(pyrazol-1-yl)imidazo[4,5-b]pyridin-3-yl]-3-oxo-1,2-dihydroinden-1-yl]-3-formyl-4-hydroxybenzamide

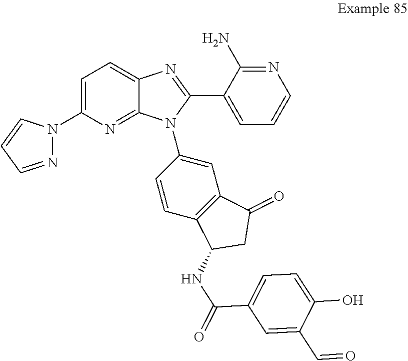 [SMILES: Nc1ncccc1-c1nc2ccc(-n3cccn3)nc2n1-c1ccc2c(c1)C(=O)C[C@@H]2NC(=O)c1ccc(O)c(C=O)c1]

Example 85 was prepared in a manner analogous to Example 12 using Intermediate 85-2 in place of Intermediate 1-3 and Intermediate 57-2 in place of Intermediate 12-1. MS (ESI) calculated for C₃₁H₂₂N₈O₄: 570.18 m/z, found 571.20 [M+H]⁺. ¹H NMR (400 MHz, DMSO-d₆) δ (ppm): 10.28 (s, 1H), 8.33-8.37 (m, 2H), 8.22 (s, 1H), 7.95-8.01 (m, 3H), 7.74-7.84 (m, 4H), 7.35-7.36 (m, 1H), 7.03-7.04 (m, 1H), 6.50-6.55 (m, 1H), 6.47-6.49 (m, 1H), 5.82-5.83 (m, 1H), 3.18-3.25 (m, 1H), 2.68-2.77 (m, 1H).

### Intermediate 85-2: (S)-3-amino-6-(2-(2-aminopyridin-3-yl)-5-(1H-pyrazol-1-yl)-3H-imidazo[4,5-b]pyridin-3-yl)-2,3-dihydro-1H-inden-1-one

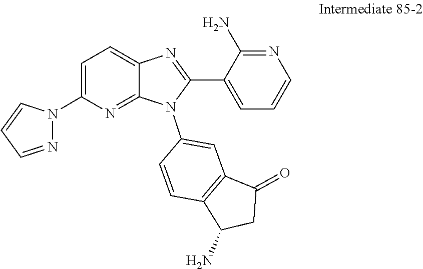 [SMILES: Nc1ncccc1-c1nc2ccc(-n3cccn3)nc2n1-c1ccc2c(c1)C(=O)C[C@@H]2N]

Intermediate 85-2 was prepared in a manner analogous to Intermediate 13-2 using Intermediate 74-1 in place of Intermediate 13-1, Intermediate 85-1 in place of 6-methyl-3-nitropyridin-2-amine and tris(dibenzylideneacetone)dipalladium(0)/dimethylacetamide in place of palladium(II) acetate/1,4-dioxane. MS (ESI) calculated for C₂₃H₁₈N₈O: 422.16 m/z, found 423.10 [M+H]⁺.

### Intermediate 85-1: 3-nitro-6-(1H-pyrazol-1-yl)pyridin-2-amine

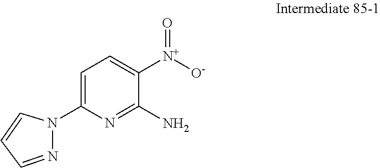 [SMILES: Nc1nc(-n2cccn2)ccc1[N+](=O)[O-]]

Intermediate 85-1 was prepared in a manner analogous to Intermediate 76-1 using pyrazole in place of 4-fluoropyrazole, 6-bromo-3-nitropyridin-2-amine in place of Intermediate 18-1 and 1,4-dioxane in place of N,N-dimethylacetamide.

### Example 86: N-[(1S)-5-[2-(2-aminopyridin-3-yl)-5-(pyrazol-1-yl)imidazo[4,5-b]pyridin-3-yl]-2,3-dihydro-1H-inden-1-yl]-3-formyl-4-methanesulfonamidobenzamide

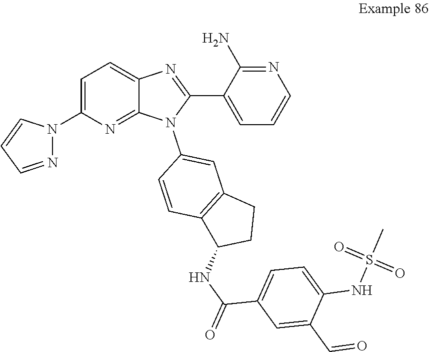 [SMILES: CS(=O)(=O)Nc1ccc(C(=O)N[C@H]2CCc3cc(-n4c(-c5cccnc5N)nc5ccc(-n6cccn6)nc54)ccc32)cc1C=O]

Example 86 was prepared in a manner analogous to Example 12 using Intermediate 33-1 in place of Intermediate 1-3, Intermediate 51-1 in place of Intermediate 12-1 and dichloromethane/2,2,2-trifluoroacetic acid (10:1) in place of 2,2,2-trifluoroacetic acid/methanesulfonic acid. MS (ESI) calculated for C₃₂H₂₇N₉O₄S: 633.159 m/z, found 634.15 [M+H]⁺. ¹H NMR (300 MHz, DMSO-d₆) δ (ppm): 10.06 (s, 1H), 8.43-8.48 (m, 2H), 8.23-8.26 (m, 1H), 7.99-8.05 (m, 2H), 7.83 (s, 1H), 7.63-7.66 (m, 2H), 7.39-7.45 (m, 4H), 6.73-6.75 (m, 1H), 6.58 (s, 1H), 5.62-5.68 (m, 1H), 3.12 (s, 3H), 2.95-3.07 (m, 2H), 2.53 (s, 1H), 2.09-2.15 (m, 1H).

### Example 87: (S)—N-(5-(2-(2-aminopyridin-3-yl)-5-(cyclopropylamino)-3H-imidazo[4,5-b]pyridin-3-yl)-2,3-dihydro-1H-inden-1-yl)-3-formyl-4-hydroxybenzamide

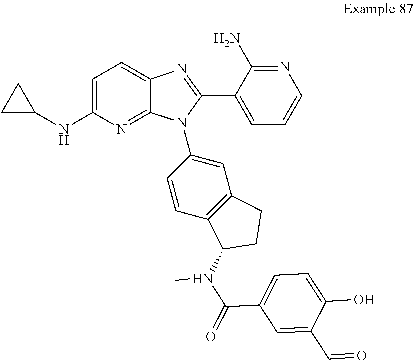 [SMILES: CN(C(=O)c1ccc(O)c(C=O)c1)[C@H]1CCc2cc(-n3c(-c4cccnc4N)nc4ccc(NC5CC5)nc43)ccc21]

Example 87 was prepared in a manner analogous to Example 12 using Intermediate 87-1 in place of Intermediate 1-3 and Intermediate 57-2 in place of Intermediate 12-1. MS (ESI) calculated for C₃₁H₂₇N₇O₃: 545.22 m/z, found 546.25 [M+H]⁺. ¹H NMR (400 MHz, DMSO-d₆) δ (ppm): 10.29 (s, 1H), 8.26-8.27 (m, 1H), 8.05-8.08 (m, 1H), 7.94-7.98 (m, 2H), 7.58-7.60 (m, 1H), 7.30-7.37 (m, 3H), 7.20-7.22 (m, 1H), 7.07-7.09 (m, 1H), 6.74-6.78 (m, 2H), 5.54-5.58 (m, 1H), 2.85-3.04 (m, 3H), 2.47-2.51 (m, 1H), 2.07-2.12 (m, 1H), 0.69-0.71 (m, 2H), 0.48-0.51 (m, 2H).

### Intermediate 87-1: (S)-3-(1-amino-2,3-dihydro-1H-inden-5-yl)-2-(2-aminopyridin-3-yl)-N-cyclopropyl-3H-imidazo[4,5-b]pyridin-5-amine

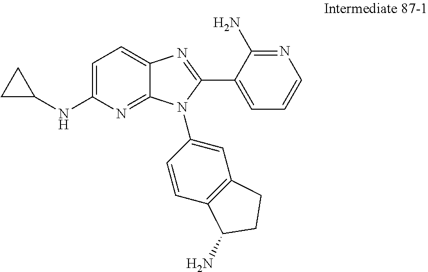 [SMILES: Nc1ncccc1-c1nc2ccc(NC3CC3)nc2n1-c1ccc2c(c1)CC[C@@H]2N]

### Synthetic Route:

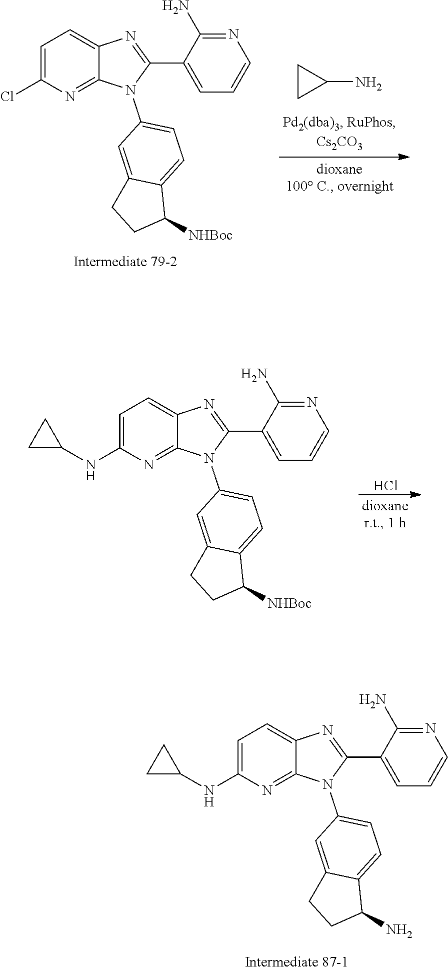

### Step 1: Synthesis of tert-butyl (S)-(5-(2-(2-aminopyridin-3-yl)-5-(cyclopropylamino)-3H-imidazo[4,5-b]pyridin-3-yl)-2,3-dihydro-1H-inden-1-yl)carbamate

A mixture of tert-butyl (S)-(5-(2-(2-aminopyridin-3-yl)-5-chloro-3H-imidazo[4,5-b]pyridin-3-yl)-2,3-dihydro-1H-inden-1-yl)carbamate (Intermediate 79-2) (500 mg, 1.05 mmol, 1 equiv), aminocyclopropane (299 mg, 5.24 mmol, 5 equiv), tris(dibenzylideneacetone)dipalladium(0) (96 mg, 0.11 mmol, 0.1 equiv), RuPhos (98 mg, 0.21 mmol, 0.2 equiv), 1,4-dioxane (5 mL) and cesium carbonate (854 mg, 2.62 mmol, 2.5 equiv) were stirred under nitrogen atmosphere at 100° C. for 3 h. The resulting mixture was purified by reverse-phase flash column chromatography on C18 silica gel using a 0-80% gradient of acetonitrile in water (+0.05% ammonium bicarbonate) to provide tert-butyl (S)-(5-(2-(2-aminopyridin-3-yl)-5-(cyclopropylamino)-3H-imidazo[4,5-b]pyridin-3-yl)-2,3-dihydro-1H-inden-1-yl)carbamate (45 mg, 5%) as a yellow solid. MS (ESI) calculated for C₂₈H₃₁N₇O₂: 497.25 m/z, found 498.15 [M+H]⁺.

### Step 2: Synthesis of (S)-3-(1-amino-2,3-dihydro-1H-inden-5-yl)-2-(2-aminopyridin-3-yl)-N-cyclopropyl-3H-imidazo[4,5-b]pyridin-5-amine (Intermediate 87-1)

(S)-(5-(2-(2-aminopyridin-3-yl)-5-(cyclopropylamino)-3H-imidazo[4,5-b]pyridin-3-yl)-2,3-dihydro-1H-inden-1-yl)carbamate (45 mg, 0.058 mmol, 1 equiv) was dissolved in 4N hydrochloric acid in 1,4-dioxane and the resulting mixture was stirred at room temperature for 1 h. The mixture was then concentrated in vacuo to provide crude (S)-3-(1-amino-2,3-dihydro-1H-inden-5-yl)-2-(2-aminopyridin-3-yl)-N-cyclopropyl-3H-imidazo[4,5-b]pyridin-5-amine (Intermediate 87-1) as a yellow solid, which was used in subsequent transformations without further purification. MS (ESI) calculated for C₂₃H₂₃N₇: 397.20 m/z, found 398.15 [M+H]⁺.

### Example 88: N-[(1S)-5-[2-(2-aminopyridin-3-yl)-5-(1,3-thiazol-4-yl)imidazo[4,5-b]pyridin-3-yl]-2,3-dihydro-1H-inden-1-yl]-3-formyl-4-hydroxybenzamide

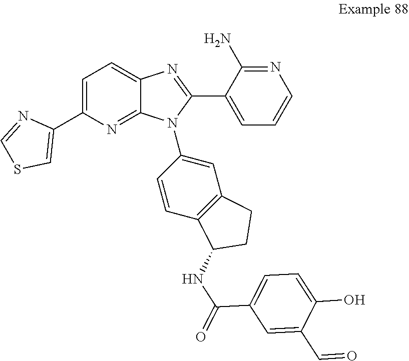 [SMILES: Nc1ncccc1-c1nc2ccc(-c3cscn3)nc2n1-c1ccc2c(c1)CC[C@@H]2NC(=O)c1ccc(O)c(C=O)c1]

Example 88 was prepared in a manner analogous to Example 12 using Intermediate 88-1 in place of Intermediate 1-3 and Intermediate 57-2 in place of Intermediate 12-1. MS (ESI) calculated for C₃₁H₂₃N₇O₃S: 573.16 m/z, found 574.15 [M+H]⁺. ¹H NMR (400 MHz, DMSO-d₆) δ (ppm): 10.30 (s, 1H), 9.20 (s, 1H), 8.30-8.40 (m, 2H), 8.20-8.30 (m, 1H), 8.00-8.29 (m, 3H), 7.60-7.80 (m, 1H), 7.40-7.50 (m, 1H), 7.30-7.40 (m, 2H), 7.00-7.10 (m, 1H), 6.65-6.85 (m, 1H), 5.50-5.70 (m, 1H), 3.00-3.10 (m, 1H), 2.85-3.00 (m, 1H), 2.60-2.65 (m, 1H), 2.00-2.20 (m, 1H).

### Intermediate 88-1: (S)-3-(3-(1-amino-2,3-dihydro-1H-inden-5-yl)-5-(thiazol-4-yl)-3H-imidazo[4,5-b]pyridin-2-yl)pyridin-2-amine

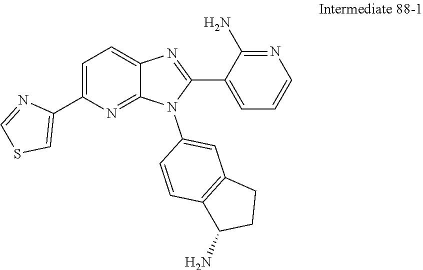 [SMILES: Nc1ncccc1-c1nc2ccc(-c3cscn3)nc2n1-c1ccc2c(c1)CC[C@@H]2N]

Intermediate 88-1 was prepared in a manner analogous to Intermediate 18-3 using Intermediate 79-1 in place of Intermediate 18-2, 4-(tributylstannyl)-1,3-thiazole in place of 3-(tributylstannyl)pyridazine and bis(triphenylphosphine)palladium(II) dichloride/copper (I) iodide/toluene/overnight in place of tetrakis(triphenylphosphine)palladium(0)/lithium chloride/N,N-dimethylformamide/3 h. MS (ESI) calculated for C₂₃H₁₉N₇S: 425.14 m/z, found 426.10 [M+H]⁺.

### Example 89: (S)—N-(5-(2-(2-aminopyridin-3-yl)-5-phenyl-3H-imidazo[4,5-b]pyridin-3-yl)-2,3-dihydro-1H-inden-1-yl)-6-formyl-5-hydroxypicolinamide

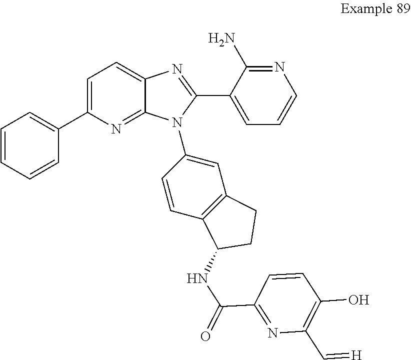

Example 89 was prepared in a manner analogous to Example 12 using Intermediate 89-1 in place of Intermediate 12-1. MS (ESI) calculated for C₃₃H₂₅N₇O₃: 567.20 m/z, found 568.20 [M+H]⁺. ¹H NMR (400 MHz, DMSO-d₆) δ (ppm): 10.15 (s, 1H), 8.18-8.45 (m, 2H), 7.91-8.16 (m, 4H), 7.59-7.84 (m, 2H), 7.28-7.56 (m, 6H), 6.81 (s, 1H), 5.35-5.81 (m, 1H), 2.82-3.11 (m, 2H), 1.98-2.45 (m, 2H).

### Intermediate 89-1: 5-(benzyloxy)-6-(1,3-dioxolan-2-yl)picolinic acid

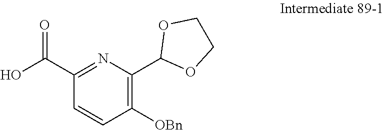 [SMILES: O=C(O)c1ccc(OCc2ccccc2)c(C2OCCO2)n1]

### Synthetic Route:

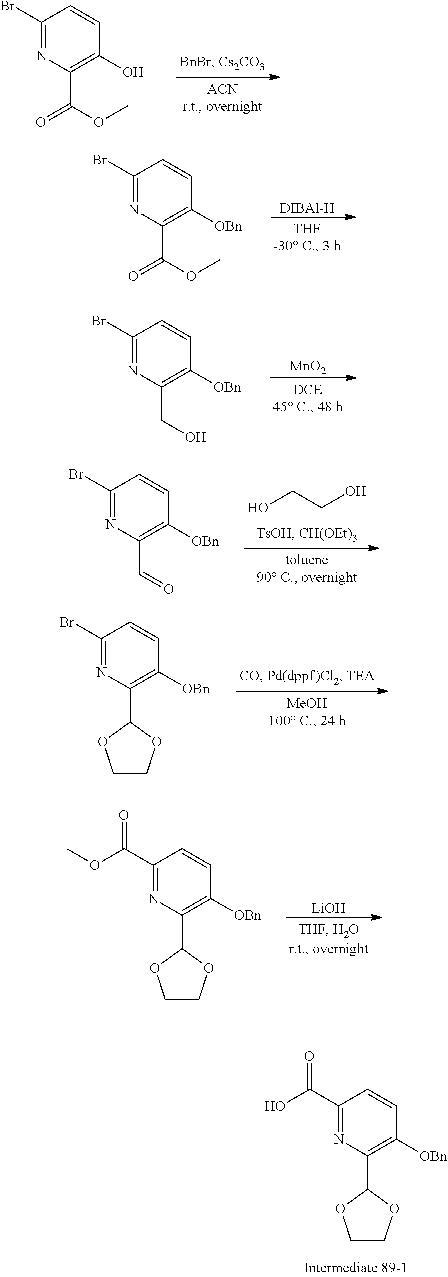

### Step 1: Synthesis of methyl 3-(benzyloxy)-6-bromopicolinate

Methyl 6-bromo-3-hydroxypyridine-2-carboxylate (5.00 g, 21.5 mmol, 1 equiv) was dissolved in acetonitrile (120 mL). Cesium carbonate (21.06 g, 64.65 mmol, 3 equiv) and benzyl bromide (4.05 g, 23.7 mmol, 1.1 equiv) were added and the mixture was stirred at room temperature for 3 h. The resulting mixture was filtered, washing with petroleum ether (150 mL). The filtrate was concentrated under reduced pressure and the residue was purified by silica gel column chromatography eluting with 20% ethyl acetate in petroleum ether to afford methyl 3-(benzyloxy)-6-bromopyridine-2-carboxylate (6.4 g, 88%) as a white solid. MS (ESI) calculated for C₁₄H₁₂BrNO₃: 321.00 m/z, found 322.85 [M+H]⁺.

### Step 2: Synthesis of (3-(benzyloxy)-6-bromopyridin-2-yl)methanol

To a cooled (−30° C.) solution of methyl 3-(benzyloxy)-6-bromopyridine-2-carboxylate (5.00 g, 15.5 mmol, 1 equiv) in tetrahydrofuran (200 mL) was added diisobutylaluminum hydride (55 mL, 47 mmol, 3 equiv) and the mixture and stirred for 3 h at −30° C. under nitrogen atmosphere. The reaction was quenched with aqueous potassium sodium tartrate and methanol and stirring was continued for 20 min at room temperature. The resulting mixture was extracted with ethyl acetate (500 mL×3). The combined organic layers were washed with brine, dried over anhydrous sodium sulfate, filtered, and concentrated under reduced pressure. The residue was purified by silica gel column chromatography eluting with 20% ethyl acetate in petroleum ether to afford [3-(benzyloxy)-6-bromopyridin-2-yl]methanol (4 g, 88%) as an off-white solid. MS (ESI) calculated for C₁₃H₁₂BrNO₂: 293.01 m/z, found 294.00 [M+H]⁺.

### Step 3: Synthesis of 3-(benzyloxy)-6-bromopicolinaldehyde

To a solution of (3-(benzyloxy)-6-bromopyridin-2-yl)methanol (3.00 g, 13.9 mmol, 1 equiv) in 1,2-dichloroethane (80 mL) was added manganese (IV) oxide (24.23 g, 278.7 mmol, 20 equiv) and the mixture was stirred for 48 h at 45° C. The reaction mixture was cooled to room temperature and filtered, rinsing with dichloromethane. The filtrate was concentrated in vacuo and the residue was purified by silica gel column chromatography eluting with 50% ethyl acetate in petroleum ether to afford 3-(benzyloxy)-6-bromopyridine-2-carbaldehyde (2.5 g, 61%) as a yellow solid. MS (ESI) calculated for C₁₃H₁₀BrNO₂: 290.99 m/z, found 291.85 [M+H]⁺.

### Step 4: Synthesis of 3-(benzyloxy)-6-bromo-2-(1,3-dioxolan-2-yl)pyridine

To a mixture of 3-(benzyloxy)-6-bromopyridine-2-carbaldehyde (3.40 g, 11.6 mmol, 1 equiv) and ethylene glycol (3.61 g, 58.2 mmol, 5 equiv) in toluene (200 mL) were added p-toluenesulfonic acid (0.20 g, 1.2 mmol, 0.1 equiv) and triethyl orthoformate (5.18 g, 34.9 mmol, 3 equiv). The resulting mixture was stirred overnight at 100° C. The reaction was cooled to room temperature and quenched with water (250 mL). The resulting mixture was extracted with ethyl acetate (400 mL×3). The combined organic layers were washed with brine (500 mL), dried over anhydrous sodium sulfate, filtered, and concentrated under reduced pressure. The residue was purified by silica gel column chromatography eluting with 20% ethyl acetate in petroleum ether to afford 3-(benzyloxy)-6-bromo-2-(1,3-dioxolan-2-yl) pyridine (2.2 g, 56%) as a yellow solid. MS (ESI) calculated for C₁₅H₁₄BrNO₃: 335.02 m/z, found 336.05 [M+H]⁺.

### Step 5: Synthesis of methyl 5-(benzyloxy)-6-(1,3-dioxolan-2-yl)picolinate

A mixture of 3-(benzyloxy)-6-bromo-2-(1,3-dioxolan-2-yl) pyridine (2.20 g, 3.27 mmol, 1 equiv), triethylamine (1.32 g, 13.1 mmol, 4 equiv) and [1,1′-bis(diphenylphosphino)ferrocene]dichloropalladium(II)dichloromethane complex (0.27 g, 0.33 mmol, 0.1 equiv) in methanol (100 mL) was sparged with nitrogen for 5 min then pressurized to 20 atm with carbon monoxide at 90° C. overnight. The reaction mixture was cooled to room temperature and filtered. The filtrate was concentrated in vacuo and the residue was purified by silica gel column chromatography, eluting with 33% ethyl acetate in petroleum ether to afford methyl 5-(benzyloxy)-6-(1,3-dioxolan-2-yl) pyridine-2-carboxylate (0.8 g, 71%) as a yellow solid. MS (ESI) calculated for C₁₇H₁₇NO₅: 315.11 m/z, found 316.10 [M+H]⁺.

### Step 6: Synthesis of 5-(benzyloxy)-6-(1,3-dioxolan-2-yl) picolinic acid

To a solution of methyl 5-(benzyloxy)-6-(1,3-dioxolan-2-yl) pyridine-2-carboxylate (200 mg, 0.634 mmol, 1 equiv) in tetrahydrofuran (8 mL) and methanol (2 mL) was added a solution of lithium hydroxide (30 mg, 1.3 mmol, 2 equiv) in water (2 mL). The mixture stirred for 1 h at room temperature. The pH of the mixture was brought to ˜3 with 1M hydrochloric acid. The resulting mixture was concentrated under reduced pressure and the residue was purified by reverse-phase flash chromatography on C18 silica gel using a 40-50% gradient of acetonitrile in water (+0.1 mmol/L 2,2,2-trifluoroacetic acid) to afford 5-(benzyloxy)-6-(1,3-dioxolan-2-yl) pyridine-2-carboxylic acid (Intermediate 89-1) (120 mg, 60%) as a yellow oil. MS (ESI) calculated for C₁₆H₁₅NO₅: 301.10 m/z, found 302.05 [M+H]⁺.

### Example 90: N-[(1S)-5-[2-(2-aminopyridin-3-yl)-5-(4-cyanopyrazol-1-yl)imidazo[4,5-b]pyridin-3-yl]-2,3-dihydro-1H-inden-1-yl]-3-formyl-4-hydroxybenzamide

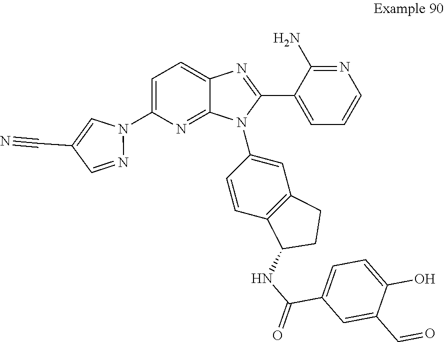 [SMILES: N#Cc1cnn(-c2ccc3nc(-c4cccnc4N)n(-c4ccc5c(c4)CC[C@@H]5NC(=O)c4ccc(O)c(C=O)c4)c3n2)c1]

Example 90 was prepared in a manner analogous to Example 12 using Intermediate 90-1 in place of Intermediate 1-3 and Intermediate 57-2 in place of Intermediate 12-1. MS (ESI) calculated for C₃₂H₂₃N₉O₃: 581.19 m/z, found 582.20 [M+H]⁺. ¹H NMR (400 MHz, DMSO-d₆) δ (ppm): 10.28 (s, 1H), 9.11 (s, 1H), 8.91 (s, 1H), 8.38-8.43 (m, 2H), 8.27 (s, 1H), 7.96-8.02 (m, 3H), 7.34-7.37 (m, 2H), 7.28-7.30 (m, 2H), 7.01 (s, 1H), 6.45-6.48 (m, 1H), 5.60-5.62 (m, 1H), 3.01-3.03 (m, 1H), 2.89-2.92 (m, 1H), 2.51-2.55 (m, 1H), 2.08-2.09 (m, 1H).

### Intermediate 90-1: (S)-1-(3-(1-amino-2,3-dihydro-1H-inden-5-yl)-2-(2-aminopyridin-3-yl)-3H-imidazo[4,5-b]pyridin-5-yl)-1H-pyrazole-4-carbonitrile

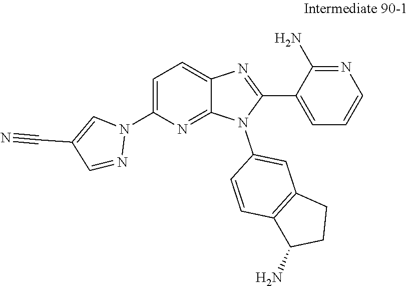 [SMILES: N#Cc1cnn(-c2ccc3nc(-c4cccnc4N)n(-c4ccc5c(c4)CC[C@@H]5N)c3n2)c1]

Intermediate 90-1 was prepared in a manner analogous to Intermediate 33-1 using 1H-pyrazole-4-carbonitrile in place of pyrazole, Intermediate 79-2 in place of Intermediate 18-2, 2 h instead of overnight (for step 1) and 4N hydrochloric acid in 1,4-dioxane at room temperature for 1 h instead of hydrochloric acid in methanol at 90° C. overnight. MS (ESI) calculated for C₂₄H₁₉N₉: 433.18 m/z, found 434.20 [M+H]⁺.

### Example 91: N-[(1S)-5-[2-(2-aminopyridin-3-yl)-5-(1,2,3-triazol-2-yl)imidazo[4,5-b]pyridin-3-yl]-2,3-dihydro-1H-inden-1-yl]-3-formyl-4-hydroxybenzamide

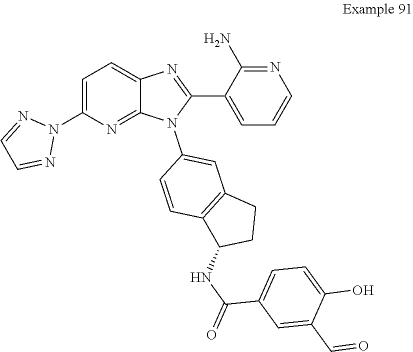 [SMILES: Nc1ncccc1-c1nc2ccc(-n3nccn3)nc2n1-c1ccc2c(c1)CC[C@@H]2NC(=O)c1ccc(O)c(C=O)c1]

Example 91 was prepared in a manner analogous to Example 12 using Intermediate 91-2 in place of Intermediate 1-3 and Intermediate 57-2 in place of Intermediate 12-1. MS (ESI) calculated for C₃₀H₂₃N₉O₃: 557.19 m/z, found 558.25 [M+H]⁺. ¹H NMR (400 MHz, DMSO-d₆) δ (ppm): 10.28 (s, 1H), 8.96-8.98 (m, 1H), 8.41-8.43 (m, 1H), 8.29 (s, 1H), 8.00-8.29 (m, 5H), 7.29-7.39 (m, 4H), 7.02-7.04 (m, 1H), 5.61-5.64 (m, 1H), 2.86-3.02 (m, 2H), 2.51 (s, 1H), 2.05-2.10 (m, 1H).

### Intermediate 91-2: (S)-3-(3-(1-amino-2,3-dihydro-1H-inden-5-yl)-5-(2H-1,2,3-triazol-2-yl)-3H-imidazo[4,5-b]pyridin-2-yl)pyridin-2-amine

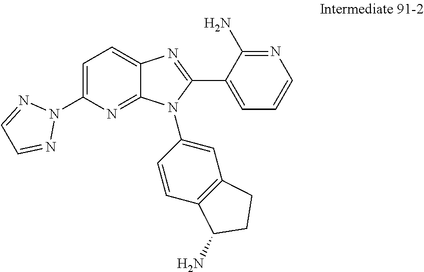 [SMILES: Nc1ncccc1-c1nc2ccc(-n3nccn3)nc2n1-c1ccc2c(c1)CC[C@@H]2N]

Intermediate 91-2 was prepared in a manner analogous to Intermediate 69-1 (starting from Step 2) using Intermediate 91-1 in of the nitro starting material. MS (ESI) calculated for C₂₂H₁₉N₉: 409.18 m/z, found 410.20 [M+H]⁺.

### Intermediate 91-1: (R)—N-(5-((3-nitro-6-(2H-1,2,3-triazol-2-yl)pyridin-2-yl)amino)-2,3-dihydro-1H-inden-1-yl)acetamide and Intermediate 92-1: (R)—N-(5-((3-nitro-6-(1H-1,2,3-triazol-1-yl)pyridin-2-yl)amino)-2,3-dihydro-1H-inden-1-yl)acetamide

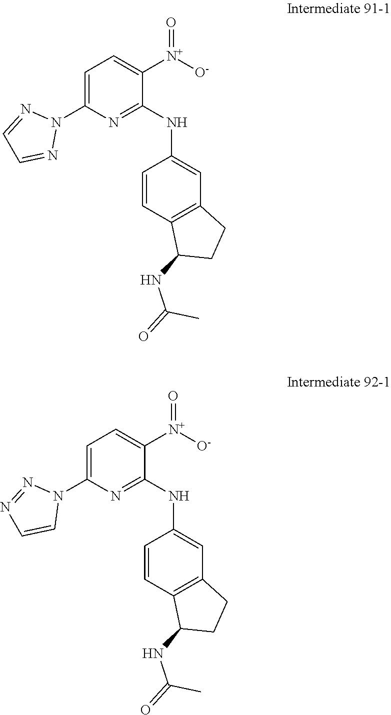 [SMILES: CC(=O)N[C@@H]1CCc2cc(Nc3nc(-n4nccn4)ccc3[N+](=O)[O-])ccc21 ; CC(=O)N[C@@H]1CCc2cc(Nc3nc(-n4ccnn4)ccc3[N+](=O)[O-])ccc21]

### Synthetic Route:

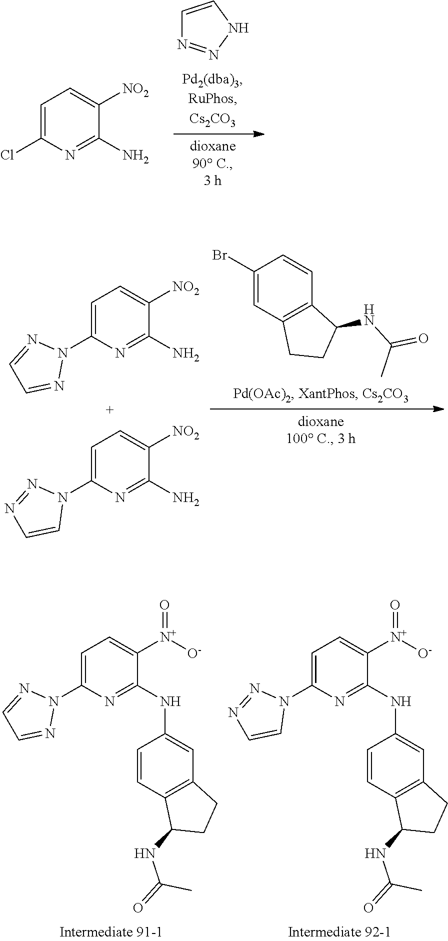

### Step 1: Synthesis of 3-nitro-6-(2H-1,2,3-triazol-2-yl)pyridin-2-amine and 3-nitro-6-(1H-1,2,3-triazol-1-yl)pyridin-2-amine

To a solution of 6-chloro-3-nitropyridin-2-amine (3.00 g, 17.3 mmol, 1 equiv) in 1,4-dioxane (36 mL) was added 1,2,3-triazole (2.39 g, 34.6 mmol, 2 equiv), tris(dibenzylideneacetone)dipalladium(0) (1.58 g, 1.73 mmol, 0.1 equiv), RuPhos (1.61 g, 3.46 mmol, 0.2 equiv) and cesium carbonate (11.26 g, 34.57 mmol, 2 equiv). The resulting mixture was stirred under nitrogen atmosphere at 90° C. for 3 h. The resulting mixture was purified by reverse-phase flash column chromatography on C18 silica gel using a 20-95% gradient of acetonitrile in water (+0.05% ammonium bicarbonate) with a 10-minute hold at 70% acetonitrile to afford a mixture of 3-nitro-6-(2H-1,2,3-triazol-2-yl)pyridin-2-amine and 3-nitro-6-(1H-1,2,3-triazol-1-yl)pyridin-2-amine (2.1 g, 59%) as a yellow solid, which was carried through to the next step without separation of the regioisomers. MS (ESI) calculated for C₇H₆N₆O₂: 206.06 m/z, found 207.10 [M+H]⁺.

### Step 2: Synthesis of (R)—N-(5-((3-nitro-6-(2H-1,2,3-triazol-2-yl)pyridin-2-yl)amino)-2,3-dihydro-1H-inden-1-yl)acetamide (Intermediate 91-2) and N-[(1S)-5-{[3-nitro-6-(1,2,3-triazol-1-yl)pyridin-2-yl]amino}-2,3-dihydro-1H-inden-1-yl]acetamide (Intermediate 92-2)

To a solution of 3-nitro-6-(2H-1,2,3-triazol-2-yl)pyridin-2-amine and 3-nitro-6-(1,2,3-triazol-1-yl)pyridin-2-amine (2.1 g (combined), 10 mmol, 1 equiv) in 1,4-dioxane (24 mL) was added N-[(1S)-5-bromo-2,3-dihydro-1H-inden-1-yl]acetamide (Intermediate 13-1) (2.59 g, 10.2 mmol, 1 equiv), palladium (II) acetate (0.23 g, 1.0 mmol, 0.1 equiv), XantPhos (0.59 g, 1.0 mmol, 0.1 equiv) and cesium carbonate (6.64 g, 20.4 mmol, 2 equiv). The resulting mixture was stirred at 100° C. under nitrogen atmosphere for 3 h then cooled to room temperature and purified by reverse-phase flash column chromatography on C18 silica gel using a 20-95% gradient of acetonitrile in water (+0.05% ammonium bicarbonate) with a 10 minute hold at 70% acetonitrile to afford (R)—N-(5-((3-nitro-6-(2H-1,2,3-triazol-2-yl)pyridin-2-yl)amino)-2,3-dihydro-1H-inden-1-yl)acetamide (Intermediate 91-1) (1.3 g, 34%) and N-[(1S)-5-{[3-nitro-6-(1,2,3-triazol-1-yl)pyridin-2-yl]amino}-2,3-dihydro-1H-inden-1-yl]acetamide (Intermediate 92-1) (1.3 g, 34%) as yellow solids.

Intermediate 91-1: MS (ESI) calculated for C₁₈H₁₇N₇O₃: 379.14 m/z, found 380.15 [M+H]⁺. ¹H NMR (400 MHz, DMSO-d₆) δ (ppm): 7.98 (s, 2H), 7.79 (d, J=2.1 Hz, 1H), 7.68 (dd, J=8.3, 2.1 Hz, 1H), 7.17 (d, J=8.1 Hz, 1H), 7.13-7.04 (m, 2H), 5.18 (t, J=7.4 Hz, 1H), 2.93-2.85 (m, 1H), 2.80-2.70 (m, 1H), 2.43-2.31 (m, 1H), 1.87 (s, 3H), 1.82-1.71 (m, 1H).

Intermediate 92-1: MS (ESI) calculated for C₁₈H₁₇N₇O₃: 379.14 m/z, found 380.15 [M+H]⁺. ¹H NMR (400 MHz, DMSO-d₆) δ (ppm): 8.37-8.38 (m, 1H), 7.87-7.89 (m, 1H), 7.49-7.53 (m, 1H), 7.46-7.48 (m, 1H), 7.24-7.26 (m, 1H), 7.08-7.14 (m, 2H), 5.17-5.21 (m, 1H), 2.88-2.95 (m, 1H), 2.73-2.79 (m, 1H), 2.33-2.41 (m, 1H), 1.87 (s, 3H), 1.80-1.86 (m, 1H).

### Example 92: N-[(1S)-5-[2-(2-aminopyridin-3-yl)-5-(1,2,3-triazol-1-yl)imidazo[4,5-b]pyridin-3-yl]-2,3-dihydro-1H-inden-1-yl]-3-formyl-4-hydroxybenzamide

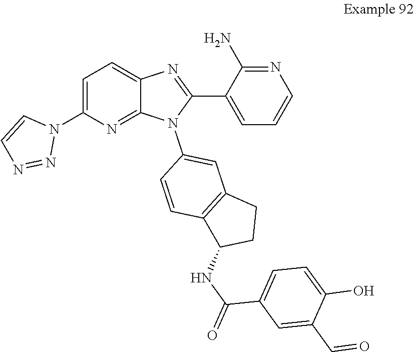 [SMILES: Nc1ncccc1-c1nc2ccc(-n3ccnn3)nc2n1-c1ccc2c(c1)CC[C@@H]2NC(=O)c1ccc(O)c(C=O)c1]

Example 92 was prepared in a manner analogous to Example 12 using Intermediate 92-2 in place of Intermediate 1-3 and Intermediate 57-2 in place of Intermediate 12-1. MS (ESI) calculated for C₃₀H₂₃N₉O₃: 557.19 m/z, found 558.20 [M+H]⁺. ¹H NMR (400 MHz, DMSO-d₆) δ (ppm): 10.28 (s, 1H), 8.96-8.98 (m, 1H), 8.41-8.43 (m, 1H), 8.29 (s, 1H), 8.00-8.29 (m, 5H), 7.29-7.39 (m, 4H), 7.02-7.04 (m, 1H), 5.61-5.64 (m, 1H), 2.86-3.02 (m, 2H), 2.51 (s, 1H), 2.05-2.10 (m, 1H).

### Intermediate 92-2: (S)-3-(3-(1-amino-2,3-dihydro-H-inden-5-yl)-5-(1H-1,2,3-triazol-1-yl)-3H-imidazo[4,5-b]pyridin-2-yl)pyridin-2-amine

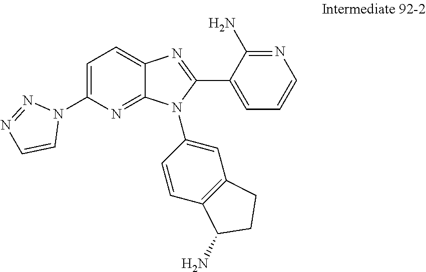 [SMILES: Nc1ncccc1-c1nc2ccc(-n3ccnn3)nc2n1-c1ccc2c(c1)CC[C@@H]2N]

Intermediate 92-2 was prepared in a manner analogous to Intermediate 69-1 (starting from Step 2) using Intermediate 92-1 (vide supra) in place the nitro starting material. MS (ESI) calculated for C₂₂H₁₉N₉: 409.18 m/z, found 410.20 [M+H]⁺.

### Example 93: N-[(1S)-5-[2-(2-aminopyridin-3-yl)-5-(pyrazol-1-yl)imidazo[4,5-b]pyridin-3-yl]-2,3-dihydro-1H-inden-1-yl]-3-chloro-5-formyl-4-hydroxybenzamide

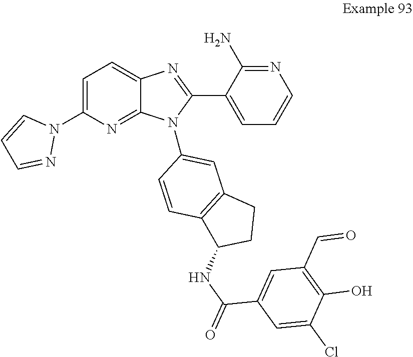 [SMILES: Nc1ncccc1-c1nc2ccc(-n3cccn3)nc2n1-c1ccc2c(c1)CC[C@@H]2NC(=O)c1cc(Cl)c(O)c(C=O)c1]

Example 93 was prepared in a manner analogous to Example 12 using Intermediate 33-1 in place of Intermediate 1-3 and Intermediate 93-1 in place of Intermediate 12-1. MS (ESI) calculated for C₃₁H₂₃ClN₈O₃: 590.16 m/z, found 591.20 [M+H]⁺. ¹H NMR (400 MHz, DMSO-d₆) δ (ppm): 10.12 (s, 1H), 8.29-8.42 (m, 2H), 8.08-8.11 (m, 2H), 7.99-8.03 (m, 1H), 7.89-7.97 (m, 1H), 7.79-7.82 (m, 1H), 7.31-7.43 (m, 2H), 7.23-7.35 (m, 2H), 6.60 (s, 1H), 6.43-6.50 (m, 1H), 5.59-5.61 (m, 1H), 2.95-3.10 (m, 2H), 2.49-2.50 (m, 1H), 2.08-2.13 (m, 1H).

### Intermediate 93-1: 4-(benzyloxy)-3-chloro-5-(1,3-dioxolan-2-yl)benzoic acid

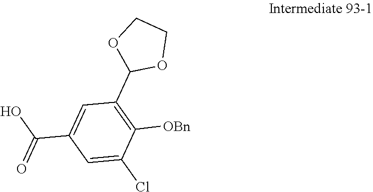 [SMILES: O=C(O)c1cc(Cl)c(OCc2ccccc2)c(C2OCCO2)c1]

### Synthetic Route:

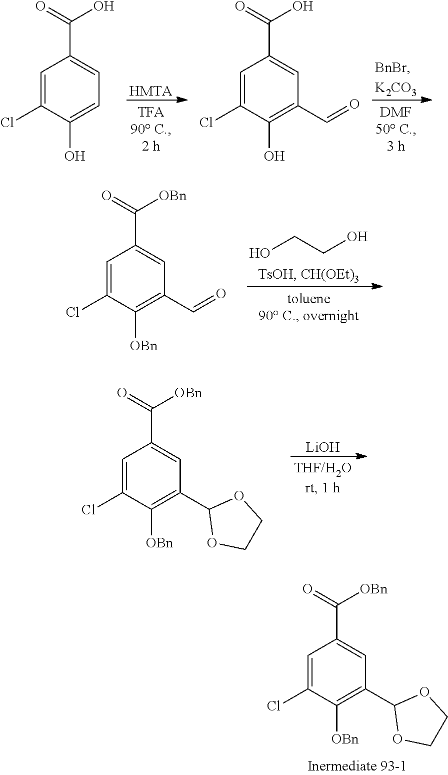

### Step 1: Synthesis of 3-chloro-5-formyl-4-hydroxybenzoic acid

A solution of 3-chloro-4-hydroxybenzoic acid (10.00 g, 57.95 mmol, 1 equiv) and 1,3,5,7-tetraazaadamantane (12.19 g, 86.93 mmol, 1.5 equiv) in 2,2,2-trifluoroacetic acid (100 mL) was stirred for 2 h at 90° C. The resulting mixture was concentrated under reduced pressure. Water (50 mL) was added, and the precipitated solids were collected by filtration and washed with water (50 mL×3) to afford 3-chloro-5-formyl-4-hydroxybenzoic acid (7 g, 54%) as a yellow solid. MS (ESI) calculated for C₈H₅ClO₄: 199.99 m/z, found 198.95 [M−H]⁻.

### Step 2: Synthesis of benzyl 4-(benzyloxy)-3-chloro-5-formylbenzoate

A solution of 3-chloro-5-formyl-4-hydroxybenzoic acid (7.00 g, 34.9 mmol, 1 equiv) and potassium carbonate (19.29 g, 139.6 mmol, 4 equiv) in N,N-dimethylformamide (70 mL) was stirred for 3 h at 50° C. The resulting mixture was extracted with ethyl acetate (300 mL×3). The combined organic layers were washed with water (300 mL×3), dried over anhydrous sodium sulfate, filtered, and concentrated under reduced pressure. The residue was purified by silica gel column chromatography using a 0-30% gradient of ethyl acetate in petroleum ether to afford benzyl 4-(benzyloxy)-3-chloro-5-formylbenzoate (7.3 g, 47%) as a yellow oil. MS (ESI) calculated for C₂₂H₁₇ClO₄: 380.08 m/z, found 421.10 [M+ACN]⁺.

### Step 3: Synthesis of benzyl 4-(benzyloxy)-3-chloro-5-(1,3-dioxolan-2-yl)benzoate

A solution of benzyl 4-(benzyloxy)-3-chloro-5-formylbenzoate (1.00 g, 2.63 mmol, 1 equiv), ethylene glycol (0.81 g, 13 mmol, 5 equiv), p-toluenesulfonic acid (0.23 g, 1.3 mmol, 0.5 equiv) and triethyl orthoformate (1.17 g, 7.88 mmol, 3 equiv) in toluene (50 mL) was stirred overnight at 90° C. The reaction was quenched with water at room temperature. The resulting mixture was extracted with ethyl acetate (200 mL×3). The combined organic layers were washed with water (200 mL×3), dried over anhydrous sodium sulfate, filtered, and concentrated under reduced pressure. The residue was purified by silica gel column chromatography using a 0-25% gradient of ethyl acetate in petroleum ether to afford benzyl 4-(benzyloxy)-3-chloro-5-(1,3-dioxolan-2-yl)benzoate (710 mg, 60%) as a yellow oil. MS (ESI) calculated for C₂₄H₂₁ClO₅: 424.11 m/z, found 425.10 [M+H]⁺.

### Step 4: Synthesis of 4-(benzyloxy)-3-chloro-5-(1,3-dioxolan-2-yl)benzoic acid (Intermediate 93-1)

A solution of benzyl 4-(benzyloxy)-3-chloro-5-(1,3-dioxolan-2-yl)benzoate (700 mg, 1.65 mmol, 1 equiv) and lithium hydroxide (118 mg, 4.94 mmol, 3 equiv) in tetrahydrofuran (15 mL), water (8 mL) and ethanol (8 mL) was stirred for 1 h at room temperature. The resulting mixture was concentrated under reduced pressure and the obtained solution was acidified to pH 6 with 2N hydrochloric acid. The resulting mixture was concentrated under reduced pressure and purified by reverse-phase flash column chromatography on C18 silica gel using a gradient of acetonitrile in water (+0.05% ammonium bicarbonate) to afford 4-(benzyloxy)-3-chloro-5-(1,3-dioxolan-2-yl)benzoic acid (Intermediate 93-1) (450 mg, 73%) as a white solid. MS (ESI) calculated for C₁₇H₁₅ClO₅: 334.06 m/z, found 335.10 [M+H]⁺.

### Example 94: N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]-3-formyl-4-hydroxybenzamide

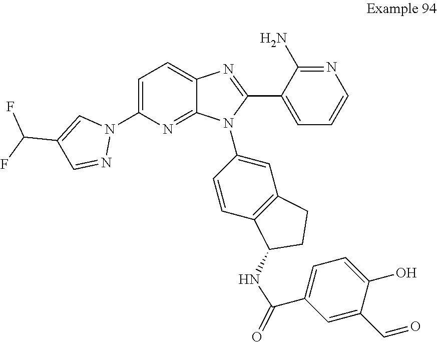 [SMILES: Nc1ncccc1-c1nc2ccc(-n3cc(C(F)F)cn3)nc2n1-c1ccc2c(c1)CC[C@@H]2NC(=O)c1ccc(O)c(C=O)c1]

### Synthetic Route:

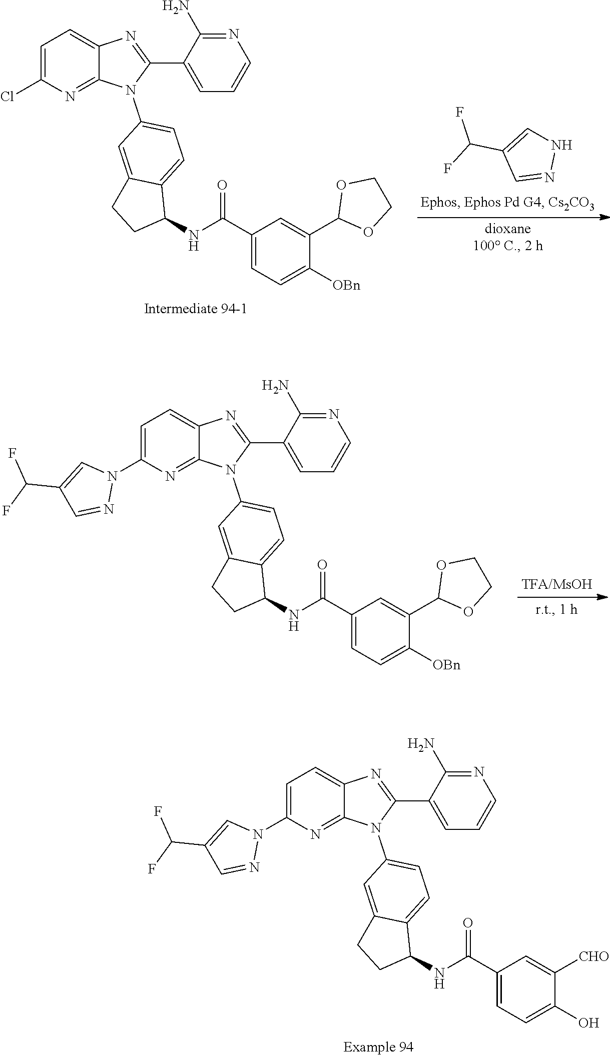 [SMILES: Nc1ncccc1-c1nc2ccc(Cl)nc2n1-c1ccc2c(c1)CC[C@@H]2NC(=O)c1ccc(OCc2ccccc2)c(C2OCCO2)c1 ; FC(F)c1cn[nH]c1 ; Nc1ncccc1-c1nc2ccc(-n3cc(C(F)F)cn3)nc2n1-c1ccc2c(c1)CC[C@@H]2NC(=O)c1ccc(OCc2ccccc2)c(C2OCCO2)c1 ; Nc1ncccc1-c1nc2ccc(-n3cc(C(F)F)cn3)nc2n1-c1ccc2c(c1)CC[C@@H]2NC(=O)c1ccc(O)c(C=O)c1]

### Step 1: Synthesis of N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]-4-(benzyloxy)-3-(1,3-dioxolan-2-yl)benzamide

To a mixture of N-[(1S)-5-[2-(2-aminopyridin-3-yl)-5-chloroimidazo[4,5-b]pyridin-3-yl]-2,3-dihydro-1H-inden-1-yl]-4-(benzyloxy)-3-(1,3-dioxolan-2-yl)benzamide (Intermediate 94-1) (150 mg, 0.23 mmol, 1 equiv) and 4-(difluoromethyl)-1H-pyrazole (81 mg, 0.68 mmol, 3 equiv) in 1,4-dioxane (3 mL) were added EPhos (24 mg, 0.046 mmol, 0.2 equiv) and EPhos Pd G4 (42 mg, 0.046 mmol, 0.2 equiv) and cesium carbonate (150 mg, 0.45 mmol, 2 equiv). The resulting mixture was stirred for 30 min at 100° C. under nitrogen atmosphere then cooled to room temperature and concentrated under reduced pressure. The residue was dissolved in N,N-dimethylformamide (1 mL) and purified by reverse-phase flash column chromatography on C18 silica gel using a 0-90% gradient of acetonitrile in water (+10 mmol/L ammonium bicarbonate) to afford N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]-4-(benzyloxy)-3-(1,3-dioxolan-2-yl)benzamide (67 mg, 32%) as a yellow solid. MS (ESI) calculated for C₄₁H₃₄F₂N₈O₄: 740.27 m/z, found 741.25 [M+H]⁺.

### Step 2: Synthesis of N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]-3-formyl-4-hydroxybenzamide (Example 94)

A solution of N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]-4-(benzyloxy)-3-(1,3-dioxolan-2-yl)benzamide (65 mg, 0.088 mmol, 1 equiv) in 2,2,2-trifluoroacetic acid (1 mL) and methanesulfonic acid (0.2 mL) was stirred for 5 min at room temperature then concentrated under reduced pressure. The residue was dissolved in N,N-dimethylformamide (1 mL) and purified by preparative HPLC on a XSelect CSH Column using a 25-50% gradient of acetonitrile in water (+0.1% formic acid) to afford N-[(1S)-5-[2-(2-aminopyridin-3-yl)-5-[4-(difluoromethyl)pyrazol-1-yl]imidazo[4,5-b]pyridin-3-yl]-2,3-dihydro-1H-inden-1-yl]-3-formyl-4-hydroxybenzamide (Example 94) (12.5 mg, 23%) as an off-white solid. MS (ESI) calculated for C₃₂H₂₄F₂N₈O₃: 606.19 m/z, found 607.20 [M+H]⁺. ¹H NMR (400 MHz, DMSO-d₆) δ (ppm): 10.30 (s, 1H), 8.66-8.67 (m, 1H), 8.47 (d, J=4.8 Hz, 1H), 8.29 (d, J=2.4 Hz, 1H), 8.03-8.10 (m, 4H), 7.81-7.83 (m, 1H) 7.45 (s, 1H), 7.31-7.39 (m, 2H), 7.07-7.12 (m, 1H), 6.82-6.86 (m, 1H), 5.61 (t, J=8.4 Hz, 1H), 2.86-3.07 (m, 2H), 2.36 (s, 2H), 2.05-2.15 (m, 1H). ¹⁹F NMR (376 MHz, DMSO-d₆) δ (ppm): −106.44.

### Intermediate 94-1: (S)—N-(5-(2-(2-aminopyridin-3-yl)-5-chloro-3H-imidazo[4,5-b]pyridin-3-yl)-2,3-dihydro-1H-inden-1-yl)-4-(benzyloxy)-3-(1,3-dioxolan-2-yl)benzamide

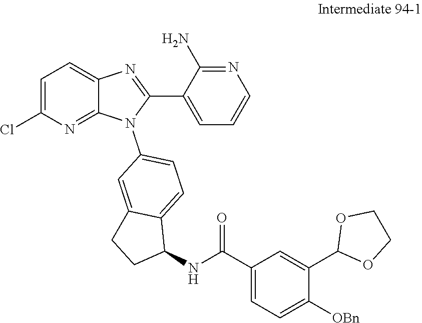 [SMILES: Nc1ncccc1-c1nc2ccc(Cl)nc2n1-c1ccc2c(c1)CC[C@@H]2NC(=O)c1ccc(OCc2ccccc2)c(C2OCCO2)c1]

### Synthetic Route:

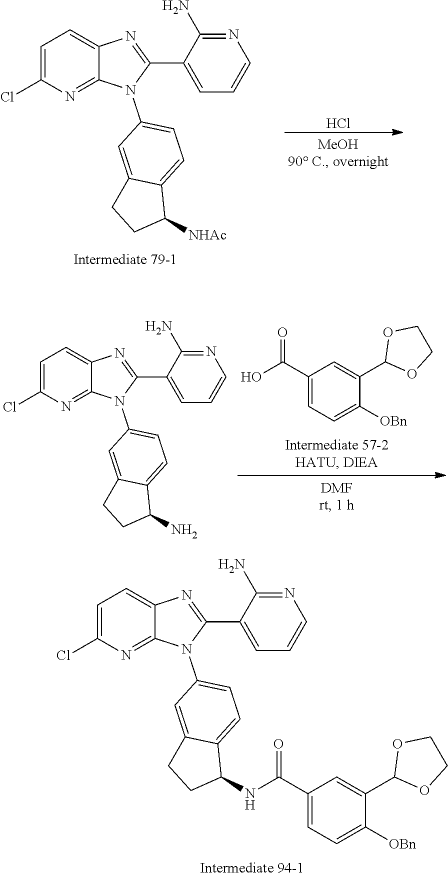

### Step 1: Synthesis of (S)-3-(3-(1-amino-2,3-dihydro-1H-inden-5-yl)-5-chloro-3H-imidazo[4,5-b]pyridin-2-yl)pyridin-2-amine

(S)—N-(5-(2-(2-aminopyridin-3-yl)-5-chloro-3H-imidazo[4,5-b]pyridin-3-yl)-2,3-dihydro-1H-inden-1-yl)acetamide (Intermediate 79-1) (200 mg, 0.478 mmol, 1 equiv) was dissolved in methanol (10 mL) and hydrochloric acid (10 mL, conc.) was added. The resulting mixture was stirred overnight at 90° C. The mixture was then cooled to room temperature and concentrated in vacuo to afford crude (S)-3-(3-(1-amino-2,3-dihydro-1H-inden-5-yl)-5-chloro-3H-imidazo[4,5-b]pyridin-2-yl)pyridin-2-amine as a yellow solid, which was used directly in the next step without further purification. MS (ESI) calculated for C₂₀H₁₇ClN₆: 376.12 m/z, found 377.15 [M+H]⁺.

### Step 2: Synthesis of (S)—N-(5-(2-(2-aminopyridin-3-yl)-5-chloro-3H-imidazo[4,5-b]pyridin-3-yl)-2,3-dihydro-1H-inden-1-yl)-4-(benzyloxy)-3-(1,3-dioxolan-2-yl)benzamide (Intermediate 94-1)

A solution of 3-{3-[(1S)-1-amino-2,3-dihydro-1H-inden-5-yl]-5-chloroimidazo[4,5-b]pyridin-2-yl}pyridin-2-amine (4.1 g, 11 mmol, 1 equiv), 4-(benzyloxy)-3-(1,3-dioxolan-2-yl)benzoic acid (Intermediate 57-1) (2.29 g, 7.62 mmol, 0.7 equiv), HATU (4.14 g, 10.9 mmol, 1 equiv) and N,N-diisopropylethylamine (2.81 g, 21.8 mmol, 2 equiv) in N,N-dimethylformamide (80 mL) was stirred for 1 h at room temperature. The reaction was quenched with water (500 mL). The resulting mixture was extracted with ethyl acetate (3×500 mL). The organic layers were combined, dried over anhydrous sodium sulfate, filtered, and concentrated in vacuo. The residue obtained was purified by reverse-phase flash column chromatography on C18 silica gel using a 20-95% gradient of acetonitrile in water (+0.05% ammonium bicarbonate) with a 10 minute hold at 70% acetonitrile to afford N-[(1S)-5-[2-(2-aminopyridin-3-yl)-5-chloroimidazo[4,5-b]pyridin-3-yl]-2,3-dihydro-1H-inden-1-yl]-4-(benzyloxy)-3-(1,3-dioxolan-2-yl)benzamide (Intermediate 94-1) (6.5 g, 77%) as a yellow solid. MS (ESI) calculated for C₃₇H₃₁ClN₆O₄: 658.21 m/z, found 659.25 [M+H]⁺.

### Example 95: N-[(1S)-5-[2-(2-aminopyridin-3-yl)-5-(3-methylpyrazol-1-yl)imidazo[4,5-b]pyridin-3-yl]-2,3-dihydro-1H-inden-1-yl]-3-formyl-4-hydroxybenzamide

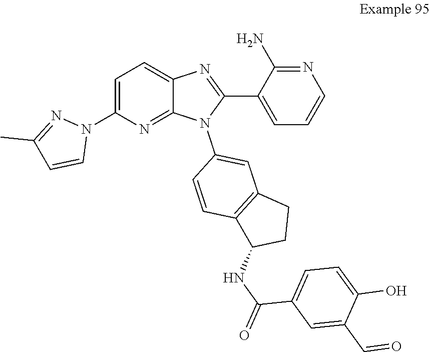 [SMILES: Cc1ccn(-c2ccc3nc(-c4cccnc4N)n(-c4ccc5c(c4)CC[C@@H]5NC(=O)c4ccc(O)c(C=O)c4)c3n2)n1]

Example 95 was prepared in a manner analogous to Example 12 using Intermediate 95-1 in place of Intermediate 1-3 and Intermediate 57-2 in place of Intermediate 12-1. MS (ESI) calculated for C₃₂H₂₆N₈O₃: 570.21 m/z, found 571.25 [M+H]⁺. ¹H NMR (400 MHz, DMSO-d₆) δ (ppm): 10.31 (s, 1H), 8.37-8.41 (m, 1H), 8.28-8.35 (m, 1H), 8.23-8.27 (m, 1H), 8.03-8.14 (m, 2H), 7.91-7.96 (m, 1H), 7.69-7.75 (m, 1H), 7.44 (s, 1H), 7.26-7.40 (m, 2H), 7.07-7.11 (m, 1H), 6.74-6.82 (m, 1H), 6.38 (d, J=2.5 Hz, 1H), 5.60-5.65 (m, 1H), 3.02-3.08 (m, 1H), 2.88-2.94 (m, 1H), 2.48-2.53 (m, 1H), 2.30 (s, 3H), 2.04-2.18 (m, 1H).

### Intermediate 95-1: (S)-3-(3-(1-amino-2,3-dihydro-1H-inden-5-yl)-5-(3-methyl-1H-pyrazol-1-yl)-3H-imidazo[4,5-b]pyridin-2-yl)pyridin-2-amine

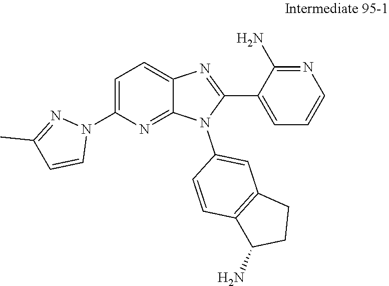 [SMILES: Cc1ccn(-c2ccc3nc(-c4cccnc4N)n(-c4ccc5c(c4)CC[C@@H]5N)c3n2)n1]

Intermediate 95-1 was prepared in a manner analogous to Intermediate 33-1 using 3-methylpyrazole in place of pyrazole and Intermediate 79-1 in place of Intermediate 18-2. MS (ESI) calculated for C₂₄H₂₂N₈: 422.20 m/z, found 423.2 [M+H]⁺.

### Example 96: (S)—N-(5-(2-(2-aminopyridin-3-yl)-5-(3-chloro-1H-pyrazol-1-yl)-3H-imidazo[4,5-b]pyridin-3-yl)-2,3-dihydro-1H-inden-1-yl)-3-formyl-4-hydroxybenzamide

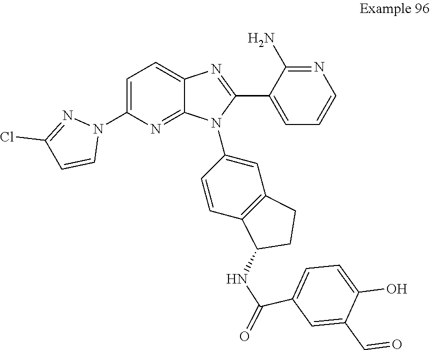 [SMILES: Nc1ncccc1-c1nc2ccc(-n3ccc(Cl)n3)nc2n1-c1ccc2c(c1)CC[C@@H]2NC(=O)c1ccc(O)c(C=O)c1]

Example 96 was prepared in a manner analogous to Example 94 using 3-chloro-1H-pyrazole in place of 4-(difluoromethyl)-1H-pyrazole. MS (ESI) calculated for C₃₁H₂₃ClN₈O₃: 590.16 m/z, found 591.10 [M+H]⁺. ¹H NMR (400 MHz, DMSO-d₆) δ (ppm): 10.31 (s, 1H), 8.96-8.98 (m, 1H), 8.39-8.40 (m, 2H), 8.37-8.39 (m, 1H), 8.31-8.32 (m, 1H), 8.12-8.14 (m, 1H), 7.85-7.87 (m, 1H), 7.35-7.40 (m, 1H), 7.28-7.33 (m, 3H), 7.07-7.09 (m, 1H), 6.46-6.49 (m, 1H), 5.61-5.65 (m, 1H), 3.00-3.03 (m, 1H), 2.90-2.92 (m, 1H), 2.50-2.51 (m, 1H), 2.07-2.09 (m, 1H).

### Example 97: N-[(1S)-5-[2-(2-aminopyridin-3-yl)-5-(1,2,4-triazol-1-yl)imidazo[4,5-b]pyridin-3-yl]-2,3-dihydro-1H-inden-1-yl]-3-formyl-4-hydroxybenzamide

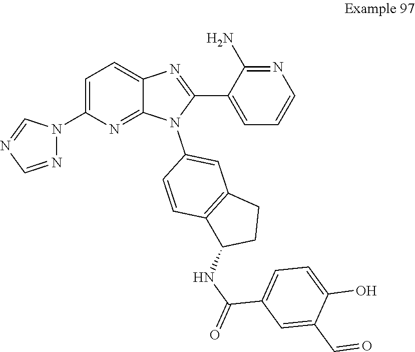 [SMILES: Nc1ncccc1-c1nc2ccc(-n3cncn3)nc2n1-c1ccc2c(c1)CC[C@@H]2NC(=O)c1ccc(O)c(C=O)c1]

Example 97 was prepared in a manner analogous to Example 12 using Intermediate 97-1 in place of Intermediate 1-3 and Intermediate 57-2 in place of Intermediate 12-1. MS (ESI) calculated for C₃₀H₂₃N₉O₃: 557.19 m/z, found 558.20 [M+H]⁺. ¹H NMR (400 MHz, DMSO-d₆) δ (ppm): 10.26 (s, 1H), 9.11 (s, 1H), 8.93 (s, 1H), 8.41-8.43 (m, 1H), 8.27-8.28 (s, 2H), 8.00-8.05 (m, 2H), 7.86-7.88 (m, 1H), 7.34-7.39 (m, 1H), 7.26-7.32 (m, 3H), 7.01-7.03 (m, 1H), 6.46-6.49 (m, 1H), 5.59-5.61 (m, 1H), 2.87-3.04 (m, 2H), 2.37-2.38 (m, 1H), 2.04-2.09 (m, 1H).

### Intermediate 97-1: 3-{3-[(1S)-1-amino-2,3-dihydro-1H-inden-5-yl]-5-(1,2,4-triazol-1-yl)imidazo[4,5-b]pyridin-2-yl}pyridin-2-amine

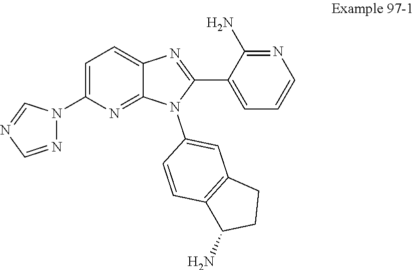 [SMILES: Nc1ncccc1-c1nc2ccc(-n3cncn3)nc2n1-c1ccc2c(c1)CC[C@@H]2N]

Intermediate 97-1 was prepared in a manner analogous to Intermediate 33-1 using Intermediate 79-1 in place of Intermediate 18-2 and 1,2,4-triazole in place of 1H-pyrazole. MS (ESI) calculated for C₂₂H₁₉N₉: 409.1 m/z, found 410.20 [M+H]⁺.

### Example 98: N-[(1S)-5-[2-(2-aminopyridin-3-yl)-5-(pyrazol-1-yl)imidazo[4,5-b]pyridin-3-yl]-3-hydroxy-2,3-dihydro-1H-inden-1-yl]-3-formyl-4-hydroxybenzamide (Diastereomer A)

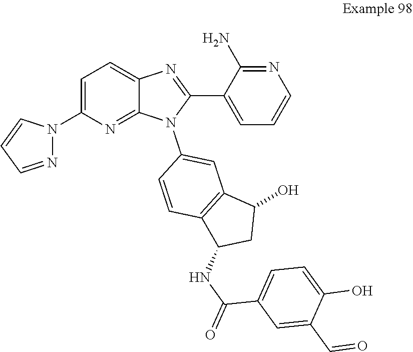 [SMILES: Nc1ncccc1-c1nc2ccc(-n3cccn3)nc2n1-c1ccc2c(c1)[C@H](O)C[C@@H]2NC(=O)c1ccc(O)c(C=O)c1]

Example 98 was prepared in a manner analogous to Example 12 using Intermediate 98-1 in place of Intermediate 1-3, Intermediate 57-2 in place of Intermediate 12-1 and TBTU/triethylamine/tetrahydrofuran in place of PyBOP/N,N-diisopropylethylamine/N,N-dimethylformamide. MS (ESI) calculated for C₃₁H₂₄N₈O₄: 572.19 m/z, found 573.15 [M+H]⁺. ¹H NMR (400 MHz, DMSO-d₆) δ (ppm): 10.31 (s, 1H), 8.37-8.39 (m, 2H), 8.27-8.37 (m, 1H), 7.99-8.12 (m, 4H), 7.75-7.82 (m, 1H), 7.51-7.53 (m, 1H), 7.31-7.41 (m, 3H), 7.07-7.10 (m, 1H), 6.53-6.57 (m, 2H), 5.04-5.85 (m, 2H), 2.83-2.86 (m, 1H), 1.95-2.05 (m, 1H). Stereochemistry of the alcohol was not determined. Depicted relative to Example 99.

### Intermediate 98-1: (3S)-3-amino-6-(2-(2-aminopyridin-3-yl)-5-(1H-pyrazol-1-yl)-3H-imidazo[4,5-b]pyridin-3-yl)-2,3-dihydro-1H-inden-1-ol (diastereomer A) and Intermediate 99-1: (3S)-3-amino-6-(2-(2-aminopyridin-3-yl)-5-(1H-pyrazol-1-yl)-3H-imidazo[4,5-b]pyridin-3-yl)-2,3-dihydro-1H-inden-1-ol (Diastereomer B)

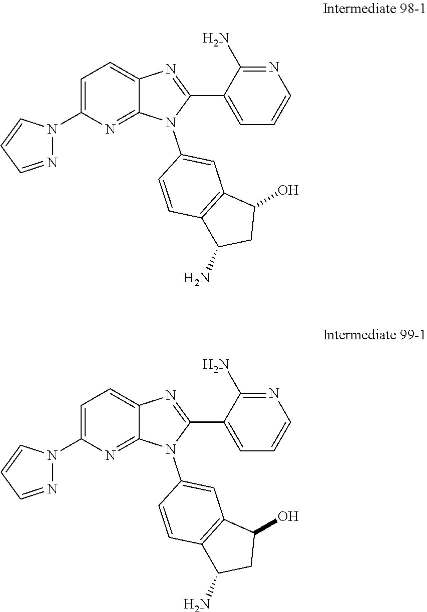 [SMILES: Nc1ncccc1-c1nc2ccc(-n3cccn3)nc2n1-c1ccc2c(c1)[C@H](O)C[C@@H]2N ; Nc1ncccc1-c1nc2ccc(-n3cccn3)nc2n1-c1ccc2c(c1)[C@@H](O)C[C@@H]2N]

### Synthetic Route:

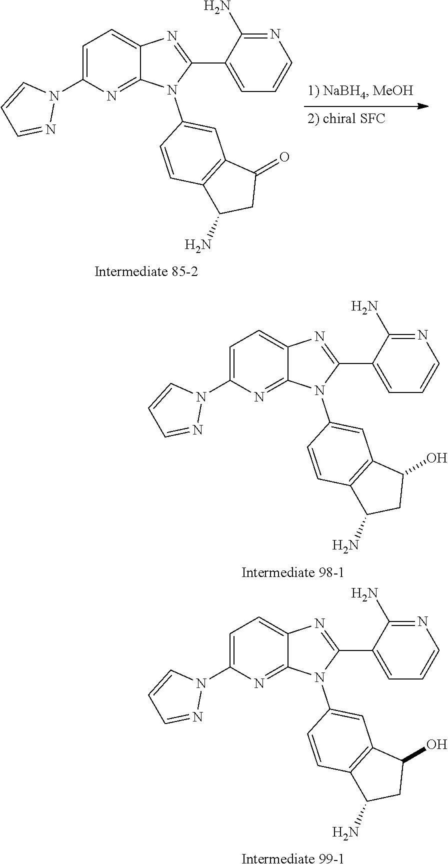 [SMILES: Nc1ncccc1-c1nc2ccc(-n3cccn3)nc2n1-c1ccc2c(c1)C(=O)C[C@@H]2N ; Nc1ncccc1-c1nc2ccc(-n3cccn3)nc2n1-c1ccc2c(c1)[C@H](O)C[C@@H]2N ; Nc1ncccc1-c1nc2ccc(-n3cccn3)nc2n1-c1ccc2c(c1)[C@@H](O)C[C@@H]2N]

### Step 1: Synthesis of 3-amino-6-[2-(2-aminopyridin-3-yl)-5-(pyrazol-1-yl)imidazo[4,5-b]pyridin-3-yl]-2,3-dihydro-1H-inden-1-ol

To a solution of (S)-3-amino-6-(2-(2-aminopyridin-3-yl)-5-(1H-pyrazol-1-yl)-3H-imidazo[4,5-b]pyridin-3-yl)-2,3-dihydro-1H-inden-1-one (Intermediate 85-2) (230 mg, 0.544 mmol, 1 equiv) in methanol (5 mL) was added sodium borohydride (41 mg, 1.1 mmol, 2 equiv). The resulting mixture was stirred at room temperature for 1 h. The reaction mixture was concentrated in vacuo and the residue was purified by reverse-phase flash column chromatography on C18 silica gel using a 0-95% gradient of acetonitrile in water (+0.05% ammonium bicarbonate) with a 10 minute hold at 50% acetonitrile to afford (3S)-3-amino-6-(2-(2-aminopyridin-3-yl)-5-(1H-pyrazol-1-yl)-3H-imidazo[4,5-b]pyridin-3-yl)-2,3-dihydro-1H-inden-1-ol (144 mg, 62%) as a yellow solid. MS (ESI) calculated for C₂₃H₂₀N₈O: 424.18 m/z, found 425.55 [M+H]⁺.

The resulting diastereomeric mixture was separated by chiral SFC on a CHIRALPAK IC-3 column eluting with a 70:30 mix of (hexanes/dichloromethane (3:1)+0.1% N,N-diisopropylethylamine):isopropanol with the first eluting peaked assigned as Intermediate 98-1 and the second eluting peak assigned as Intermediate 99-1. Absolute stereochemistry of the alcohol was not determined.

### Example 99: N-[(3S)-5-[2-(2-aminopyridin-3-yl)-5-(pyrazol-1-yl)imidazo[4,5-b]pyridin-3-yl]-3-hydroxy-2,3-dihydro-1H-inden-1-yl]-3-formyl-4-hydroxybenzamide (Diastereomer B)

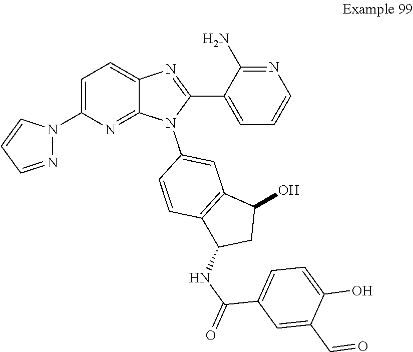 [SMILES: Nc1ncccc1-c1nc2ccc(-n3cccn3)nc2n1-c1ccc2c(c1)[C@@H](O)C[C@@H]2NC(=O)c1ccc(O)c(C=O)c1]

Example 99 was prepared in a manner analogous to Example 12 using Intermediate 99-1 (vide supra) in place of Intermediate 1-3, Intermediate 57-2 in place of Intermediate 12-1 and TBTU/triethylamine/tetrahydrofuran in place of PyBOP/N,N-diisopropylethylamine/N,N-dimethylformamide. MS (ESI) calculated for C₃₁H₂₄N₈O₄: 572.19 m/z, found 573.15 [M+H]⁺. ¹H NMR (400 MHz, DMSO-d₆) δ (ppm): 10.30 (s, 1H), 8.35-8.46 (m, 2H), 8.25-8.34 (m, 1H), 7.99-8.12 (m, 3H), 7.75-7.85 (m, 1H), 7.60-7.79 (m, 1H), 7.51-7.59 (m, 1H), 7.29-7.45 (m, 2H), 7.01-7.11 (m, 1H), 6.67-6.79 (m, 1H), 6.55-6.61 (m, 1H), 5.01-5.85 (m, 2H), 2.79-2.80 (m, 1H), 1.95-2.09 (m, 1H). Stereochemistry of the alcohol was not determined. Depicted relative to Example 98.

### Example 100: N-[(1S)-5-[2-(2-aminopyridin-3-yl)-5-(pyrazol-1-yl)imidazo[4,5-b]pyridin-3-yl]-2,3-dihydro-1H-inden-1-yl]-3-formyl-4-hydroxy-5-(trifluoromethyl)benzamide

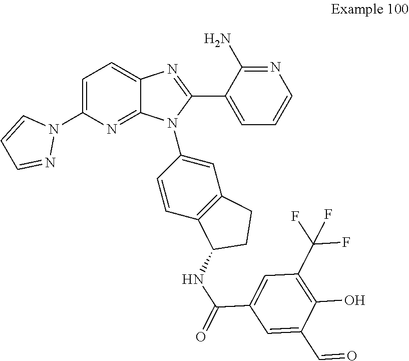 [SMILES: Nc1ncccc1-c1nc2ccc(-n3cccn3)nc2n1-c1ccc2c(c1)CC[C@@H]2NC(=O)c1cc(C=O)c(O)c(C(F)(F)F)c1]

Example 100 was prepared in a manner analogous to Example 12 using Intermediate 33-1 in place of Intermediate 1-3 and Intermediate 100-2 in place of Intermediate 12-1. MS (ESI) calculated for C₃₂H₂₃F₃N₈O₃: 624.18 m/z, found 625.25 [M+H]⁺. ¹H NMR (400 MHz, DMSO-d₆) δ (ppm): 10.14 (s, 1H), 8.64-8.65 (m, 1H), 8.47-8.48 (m, 1H), 8.36-8.41 (m, 2H), 8.03-8.05 (m, 1H), 7.97-8.00 (m, 1H), 7.80-7.81 (m, 1H), 7.61-7.63 (m, 1H), 7.38-7.43 (m, 1H), 7.31-7.33 (m, 2H), 6.68-6.71 (m, 1H), 6.55-6.56 (m, 1H), 5.62-5.66 (m, 1H), 3.02-3.08 (m, 1H), 2.88-2.96 (m, 1H), 2.56-2.58 (m, 1H), 2.06-2.10 (m, 1H). ¹⁹F NMR (376 MHz, DMSO-d₆) δ (ppm): −61.20.

### Intermediate 100-2: 4-(benzyloxy)-3-(1,3-dioxolan-2-yl)-5-(trifluoromethyl)benzoic acid

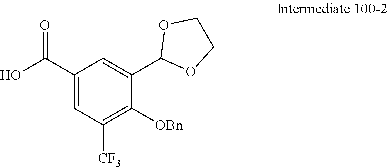 [SMILES: O=C(O)c1cc(C2OCCO2)c(OCc2ccccc2)c(C(F)(F)F)c1]

### Synthetic Route:

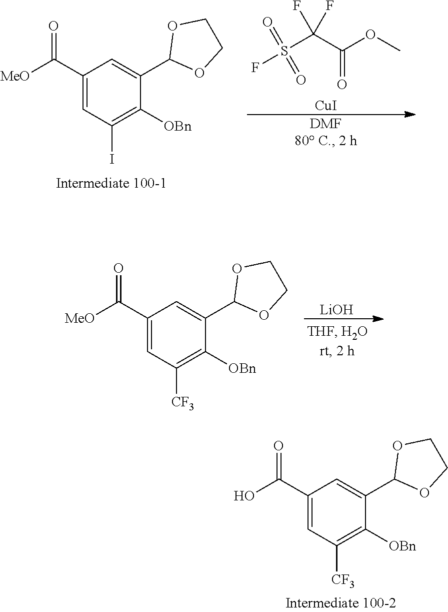

### Step 1: Synthesis of methyl 4-(benzyloxy)-3-(1,3-dioxolan-2-yl)-5-(trifluoromethyl)benzoate

To a solution of methyl 4-(benzyloxy)-3-(1,3-dioxolan-2-yl)-5-iodobenzoate (Intermediate 100-1) (500 mg, 1.63 mmol, 1 equiv) in N,N-dimethylformamide (10 mL) was added methyl 2,2-difluoro-2-(fluorosulfonyl)acetate (940 mg, 4.90 mmol, 3 equiv) and copper (I) iodide (62 mg, 0.33 mmol, 0.2 equiv). The resulting mixture was stirred for 2 h at 80° C. under a nitrogen atmosphere then cooled to 0° C. and quenched by the addition of saturated aqueous ammonium chloride (10 mL). The resulting mixture was extracted with ethyl acetate (20 mL×3). The combined organic layers were washed with water (10 mL×3), dried over anhydrous sodium sulfate, filtered, and concentrated under reduced pressure. The residue was purified by silica gel column chromatography using a 0-17% gradient of ethyl acetate in petroleum ether to afford methyl 4-(benzyloxy)-3-(1,3-dioxolan-2-yl)-5-(trifluoromethyl)benzoate (300 mg, 74%) as a white solid. MS (ESI) calculated for C₁₉H₁₇F₃O₅: 382.10 m/z, found 383.10 [M+H]⁺.

### Step 2: Synthesis of 4-(benzyloxy)-3-(1,3-dioxolan-2-yl)-5-(trifluoromethyl)benzoic acid (Intermediate 100-2)

To a cooled (0° C.) solution of methyl 4-(benzyloxy)-3-(1,3-dioxolan-2-yl)-5-(trifluoromethyl)benzoate (320 mg, 0.837 mmol, 1 equiv) in tetrahydrofuran (15 mL) was added a solution of lithium hydroxide (60 mg, 2.5 mmol, 3 equiv) in water (5 mL). The resulting solution was stirred for 2 h at room temperature then concentrated under reduced pressure. The solution was acidified to pH 6 with 2 N hydrochloric acid and the resulting precipitate was collected by filtration to give 4-(benzyloxy)-3-(1,3-dioxolan-2-yl)-5-(trifluoromethyl)benzoic acid (Intermediate 100-1) (120 mg, 39%) as a white solid. MS (ESI) calculated for C₁₈H₁₅F₃O₅: 368.09 m/z, found 369.15 [M+H]⁺.

### Intermediate 100-1: methyl 4-(benzyloxy)-3-(1,3-dioxolan-2-yl)-5-iodobenzoate

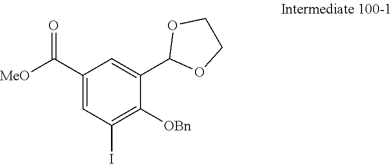 [SMILES: COC(=O)c1cc(I)c(OCc2ccccc2)c(C2OCCO2)c1]

### Synthetic Route:

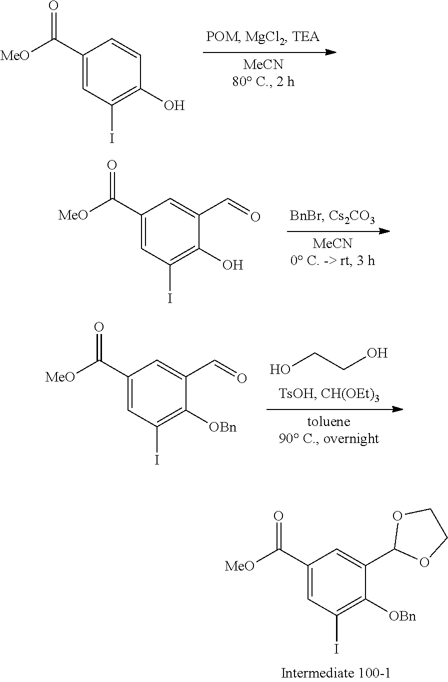

### Step 1: Synthesis of methyl 3-formyl-4-hydroxy-5-iodobenzoate

To a solution of methyl 4-hydroxy-3-iodobenzoate (100 mg, 0.360 mmol, 1 equiv) in tetrahydrofuran (5 mL) was added paraformaldehyde (162 mg, 1.80 mmol, 5 equiv), magnesium (II) chloride (51 mg, 0.54 mmol, 1.5 equiv) and triethylamine (91 mg, 0.90 mmol, 2.5 equiv). The resulting mixture was stirred at room temperature for 10 min then at 80° C. for 2 h. The mixture was then cooled to 0° C. and quenched by the addition of 2M hydrochloric acid (2 M). The mixture was extracted with ethyl acetate (20 mL×3). The combined organic layers were washed with water (20 mL×3), dried over anhydrous sodium sulfate, filtered, and concentrated under reduced pressure. The residue was purified by silica gel column chromatography using a 0-80% gradient of ethyl acetate in petroleum ether to provide methyl 3-formyl-4-hydroxy-5-iodobenzoate (20 mg, 18%) as a light-yellow solid. MS (ESI) calculated for C₉H₇IO₄: 305.94 m/z, found 304.95 [M−H]⁻.

### Step 2: Synthesis of methyl 4-(benzyloxy)-3-formyl-5-iodobenzoate

To a solution of methyl 3-formyl-4-hydroxy-5-iodobenzoate (2.00 g, 6.54 mmol, 1 equiv) in acetonitrile (40 mL) was added benzyl bromide (1.68 g, 9.80 mmol, 1.5 equiv) and cesium carbonate (4.26 g, 13.1 mmol, 2 equiv). The resulting mixture was stirred at room temperature for 3 h then cooled to 0° C. and quenched by the addition of water. The mixture was extracted with ethyl acetate (50 mL×3). The combined organic layers were washed with water (50 mL×3), dried over anhydrous sodium sulfate, filtered, and concentrated under reduced pressure. The residue was triturated with petroleum ether to afford methyl 4-(benzyloxy)-3-formyl-5-iodobenzoate (1.5 g, 58%) as a white solid. MS (ESI) calculated for C₁₆H₁₃IO₄: 395.99 m/z, found 396.95 [M+H]⁺.

### Step 3: Synthesis of methyl 4-(benzyloxy)-3-(1,3-dioxolan-2-yl)-5-iodobenzoate (Intermediate 100-1)

To a solution of methyl 4-(benzyloxy)-3-formyl-5-iodobenzoate (280 mg, 0.707 mmol, 1 equiv) in toluene (10 mL) was added ethylene glycol (219 mg, 3.54 mmol, 5 equiv), triethyl orthoformate (314 mg, 2.12 mmol, 3 equiv) and p-toluenesulfonic acid (12 mg, 0.071 mmol, 0.1 equiv). The resulting solution was stirred at room temperature for 10 min then at 90° C. overnight. The reaction was cooled to 0° C. and quenched by the addition of saturated aqueous sodium bicarbonate. The resulting mixture was extracted with ethyl acetate (20 mL×3). The combined organic layers were washed with water (20 mL×3), dried over anhydrous sodium sulfate, filtered, and concentrated under reduced pressure. The residue was purified by silica gel column chromatography using a 0-17% gradient of ethyl acetate in petroleum ether to provide methyl 4-(benzyloxy)-3-(1,3-dioxolan-2-yl)-5-iodobenzoate (Intermediate 100-1) (180 mg, 58%) as a colorless oil. MS (ESI) calculated for C₁₈H₁₇IO₅: 440.01 m/z, found 441.10 [M+H]⁺.

### Example 101: N-[(1S)-5-[2-(2-aminopyridin-3-yl)-5-(pyrazol-1-yl)imidazo[4,5-b]pyridin-3-yl]-2,3-dihydro-1H-inden-1-yl]-3-ethynyl-5-formyl-4-hydroxybenzamide

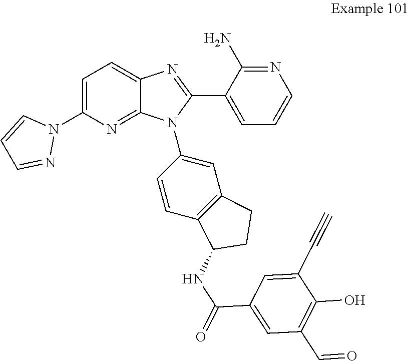 [SMILES: C#Cc1cc(C(=O)N[C@H]2CCc3cc(-n4c(-c5cccnc5N)nc5ccc(-n6cccn6)nc54)ccc32)cc(C=O)c1O]

Example 101 was prepared in a manner analogous to Example 12 using Intermediate 33-1 in place of Intermediate 1-3 and Intermediate 101-1 in place of Intermediate 12-1. MS (ESI) calculated for C₃₃H₂₄N₈O₃: 580.20 m/z, found 581.25 [M+H]⁺. ¹H NMR (400 MHz, DMSO-d₆) δ (ppm): 10.18 (s, 1H), 9.80-9.90 (m, 1H), 8.35-8.37 (m, 2H), 7.93-8.07 (m, 4H), 7.80-7.81 (m, 1H), 7.25-7.36 (m, 4H), 6.54-6.55 (m, 1H), 6.44-6.47 (m, 1H), 5.54 (s, 1H), 3.82-4.12 (m, 1H), 2.80-3.18 (m, 2H), 2.55-2.58 (m, 1H), 2.06-2.11 (m, 1H).

### Intermediate 101-1: 4-(benzyloxy)-3-(1,3-dioxolan-2-yl)-5-ethynylbenzoic acid

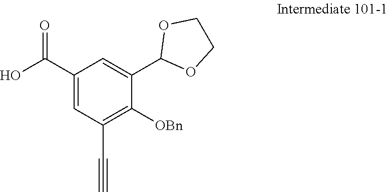 [SMILES: C#Cc1cc(C(=O)O)cc(C2OCCO2)c1OCc1ccccc1]

### Synthetic Route:

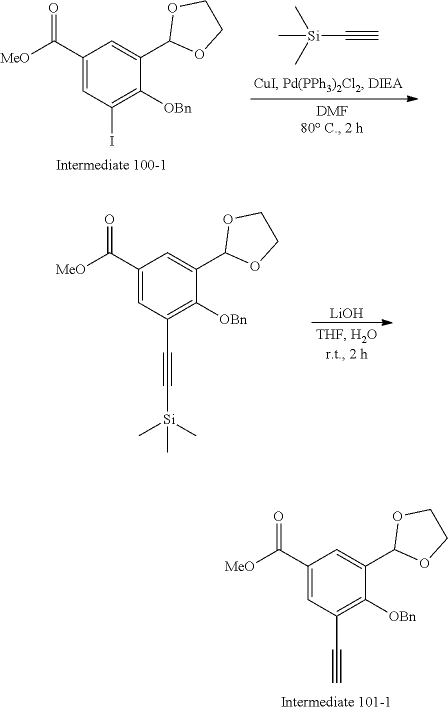

### Step 1: Synthesis of methyl 4-(benzyloxy)-3-(1,3-dioxolan-2-yl)-5-[2-(trimethylsilyl)ethynyl]benzoate

To a solution of methyl 4-(benzyloxy)-3-(1,3-dioxolan-2-yl)-5-iodobenzoate (Intermediate 100-1) (500 mg, 1.14 mmol, 1 equiv) in N,N-dimethylformamide (10 mL) was added trimethylsilylacetylene (335 mg, 3.41 mmol, 3 equiv), bis(triphenylphosphine)palladium(II) dichloride (40 mg, 0.057 mmol, 0.05 equiv), copper (I) iodide (10.82 mg, 0.057 mmol, 0.05 equiv) and N,N-diisopropylethylamine (293 mg, 2.27 mmol, 2 equiv). The resulting mixture was stirred for 2 h at 80° C. then cooled to 0° C. and quenched by the addition of saturated aqueous ammonium chloride (50 mL). The mixture was extracted with ethyl acetate (100 mL×3). The combined organic layers were washed with water (100 mL×3), dried over anhydrous sodium sulfate, filtered, and concentrated under reduced pressure. The residue was purified by silica gel column chromatography using a 0-17% gradient of ethyl acetate in petroleum ether to provide methyl 4-(benzyloxy)-3-(1,3-dioxolan-2-yl)-5-[2-(trimethylsilyl)ethynyl]benzoate (300 mg, 64%) as a light-yellow oil. MS (ESI) calculated for C₂₃H₂₆O₅Si: 410.15 m/z, found 411.25 [M+H]⁺.

### Step 2: Synthesis of 4-(benzyloxy)-3-(1,3-dioxolan-2-yl)-5-ethynylbenzoic acid (Intermediate 101-1)

To a cooled (0° C.) solution of methyl 4-(benzyloxy)-3-(1,3-dioxolan-2-yl)-5-[2-(trimethylsilyl)ethynyl]benzoate (300 mg, 0.731 mmol, 1 equiv) in tetrahydrofuran (12 mL) was added a solution of lithium hydroxide (53 mg, 2.2 mmol, 3 equiv), in water (3 mL). The resulting solution was stirred for 2 h at room temperature then concentrated under reduced pressure. The obtained solution was acidified to pH 6 with 2N hydrochloric acid and the resulting precipitate was collected by filtration to provide 4-(benzyloxy)-3-(1,3-dioxolan-2-yl)-5-ethynylbenzoic acid (Intermediate 101-1) (200 mg, 84%) as a light brown solid. MS (ESI) calculated for C₁₉H₁₆O₅: 324.10 m/z, found 325.15 [M+H]⁺.

### Example 102: (S)-3-acetyl-N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-4-hydroxybenzamide

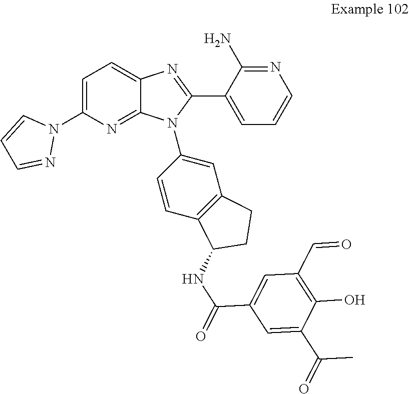 [SMILES: CC(=O)c1cc(C(=O)N[C@H]2CCc3cc(-n4c(-c5cccnc5N)nc5ccc(-n6cccn6)nc54)ccc32)cc(C=O)c1O]

Example 102 was collected as a side product from the final deprotection used for the preparation of Example 101. MS (ESI) calculated for C₃₃H₂₆N₈O₄: 598.21 m/z, found 599.25 [M+H]⁺. ¹H NMR (400 MHz, CDCl₃) δ (ppm): 13.20-13.30 (m, 1H), 10.47 (s, 1H), 8.80-8.81 (m, 1H), 8.39-8.42 (m, 2H), 8.18-8.20 (m, 1H), 8.05-8.10 (m, 2H), 7.71-7.72 (m, 1H), 7.50-7.52 (m, 1H), 7.28-7.33 (m, 2H), 7.14-7.17 (m, 1H), 6.67-6.69 (m, 1H), 6.65 (s, 2H), 6.42-6.47 (m, 2H), 5.84-5.90 (m, 1H), 3.02-3.13 (m, 2H), 2.77-2.87 (m, 4H), 2.06-2.11 (m, 1H).

### Example 103: N-[(1S)-5-[2-(2-aminopyridin-3-yl)-5-(pyrazin-2-yl)imidazo[4,5-b]pyridin-3-yl]-2,3-dihydro-1H-inden-1-yl]-2,3-difluoro-5-formyl-4-hydroxybenzamide

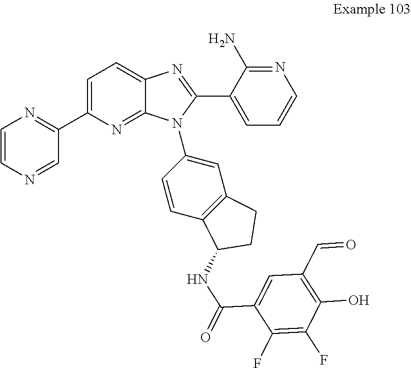 [SMILES: Nc1ncccc1-c1nc2ccc(-c3cnccn3)nc2n1-c1ccc2c(c1)CC[C@@H]2NC(=O)c1cc(C=O)c(O)c(F)c1F]

Example 103 was prepared in a manner analogous to Example 22 using Intermediate 29-1 in place of Intermediate 1-3. MS (ESI) calculated for C₃₂H₂₂F₂N₈O₃: 604.18 m/z, found 605.20 [M+H]⁺. ¹H NMR (300 MHz, DMSO-d₆) δ (ppm): 10.19 (s, 1H), 9.35 (s, 1H), 8.73-8.74 (m, 1H), 8.66-8.67 (m, 1H), 8.35-8.42 (m, 2H), 8.03-8.04 (m, 1H), 7.77-7.81 (m, 1H), 7.43-7.46 (m, 2H), 7.30-7.38 (m, 2H), 6.47-6.51 (m, 1H), 5.55-5.61 (m, 1H), 2.85-3.03 (m, 2H), 2.50-2.51 (m, 1H), 2.01-2.09 (m, 1H). ¹⁹F NMR (376 MHz, DMSO-d₆) δ (ppm): −129.34, −159.09.

### Example 104: N-[(1S)-5-[2-(2-aminopyridin-3-yl)-5-(pyrazol-1-yl)imidazo[4,5-b]pyridin-3-yl]-2,3-dihydro-1H-inden-1-yl]-4-[(dimethylcarbamoyl)amino]-3-formylbenzamide

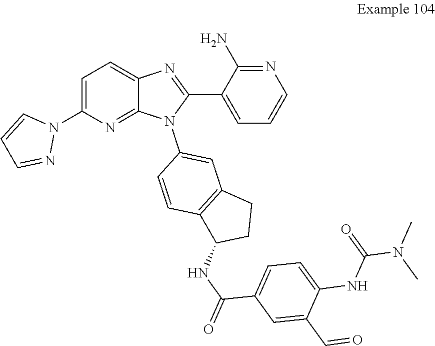 [SMILES: CN(C)C(=O)Nc1ccc(C(=O)N[C@H]2CCc3cc(-n4c(-c5cccnc5N)nc5ccc(-n6cccn6)nc54)ccc32)cc1C=O]

Example 104 was prepared in a manner analogous to Example 12 using Intermediate 33-1 in place of Intermediate 1-3 and Intermediate 63-2 in place of Intermediate 12-1. MS (ESI) calculated for C₃₄H₃₀N₁₀O₃: 626.25 m/z, found 627.30 [M+H]⁺. ¹H NMR (300 MHz, DMSO-d₆) δ (ppm): 10.00 (s, 1H), 8.48-8.51 (m, 1H), 8.43-8.44 (m, 1H), 8.36-8.38 (m, 2H), 8.12-8.18 (m, 1H), 8.00-8.05 (m, 1H), 7.95-7.98 (m, 1H), 7.82 (s, 1H), 7.39 (s, 2H), 7.30-7.31 (m, 2H), 6.58-6.59 (m, 1H), 6.57-6.58 (m, 1H), 5.60-5.69 (m, 1H), 3.04 (m, 6H), 2.85-3.00 (m, 2H), 2.50 (s, 1H), 2.05-2.15 (s, 1H).

### Example 105: N-[(1S)-5-[2-(2-aminopyridin-3-yl)-5-(pyrazol-1-yl)imidazo[4,5-b]pyridin-3-yl]-2,3-dihydro-1H-inden-1-yl]-2-fluoro-5-formyl-4-hydroxybenzamide

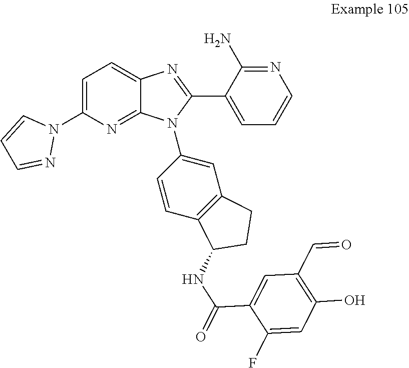 [SMILES: Nc1ncccc1-c1nc2ccc(-n3cccn3)nc2n1-c1ccc2c(c1)CC[C@@H]2NC(=O)c1cc(C=O)c(O)cc1F]

Example 105 was prepared in a manner analogous to Example 7 using Intermediate 33-1 in place of Intermediate 1-2 and Intermediate 16-1 in place of Intermediate 5-1. MS (ESI) calculated for C₃₁H₂₃FN₈O₃: 574.19 m/z, found 575.20 [M+H]⁺. ¹H NMR (400 MHz, DMSO-d₆) δ (ppm): 10.20 (s, 1H), 8.80-8.82 (m, 1H), 8.34-8.36 (m, 2H), 8.02-8.13 (m, 2H), 8.00-8.01 (m, 1H), 7.80-7.99 (m, 1H), 7.31-7.40 (m, 2H), 7.25-7.29 (m, 2H), 6.82-6.85 (m, 1H), 6.54-6.55 (m, 1H), 6.44-6.47 (m, 1H), 5.53-5.58 (m, 1H), 2.91-3.03 (m, 2H), 2.87-2.89 (m, 1H), 2.01-2.05 (m, 1H).

### Example 106: N-[(1S)-5-[2-(2-aminopyridin-3-yl)-5-(3-methoxypyrazol-1-yl)imidazo[4,5-b]pyridin-3-yl]-2,3-dihydro-1H-inden-1-yl]-3-formyl-4-hydroxybenzamide

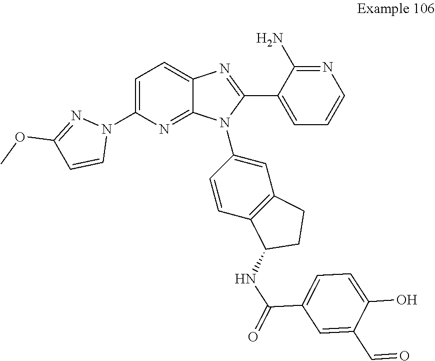 [SMILES: COc1ccn(-c2ccc3nc(-c4cccnc4N)n(-c4ccc5c(c4)CC[C@@H]5NC(=O)c4ccc(O)c(C=O)c4)c3n2)n1]

Example 106 was prepared in a manner analogous to Example 7 using Intermediate 106-1 in place of Intermediate 1-2 and Intermediate 57-2 in place of Intermediate 5-1. MS (ESI) calculated for C₃₂H₂₆N₈O₄: 586.21 m/z, found 587.25 [M+H]⁺. ¹H NMR (400 MHz, DMSO-d₆) δ (ppm): 10.30 (s, 1H), 8.96-8.98 (m, 1H), 8.26-8.27 (m, 2H), 8.24-8.25 (m, 1H), 8.14-8.16 (m, 1H), 8.02-8.05 (m, 1H), 7.72-7.76 (m, 1H), 7.20-7.31 (m, 4H), 7.04-7.06 (m, 1H), 6.47-6.48 (m, 1H), 6.45-6.46 (m, 1H), 6.03-6.04 (m, 1H), 5.54-5.58 (m, 1H), 3.86-3.87 (m, 3H), 2.97-2.99 (m, 1H), 2.86-2.88 (m, 1H), 2.50-2.51 (m, 1H), 2.03-2.06 (m, 1H).

### Intermediate 106-1: (S)-3-(3-(1-amino-2,3-dihydro-1H-inden-5-yl)-5-(3-methoxy-1H-pyrazol-1-yl)-3H-imidazo[4,5-b]pyridin-2-yl)pyridin-2-amine

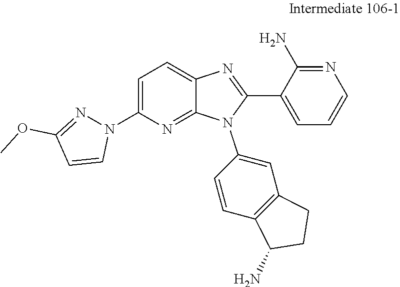 [SMILES: COc1ccn(-c2ccc3nc(-c4cccnc4N)n(-c4ccc5c(c4)CC[C@@H]5N)c3n2)n1]

Intermediate 106-1 was prepared in a manner analogous to Intermediate 33-1 using Intermediate 79-2 in place of Intermediate 18-2, 2 h instead of overnight (for Step 1) and 4N hydrochloric acid in 1,4-dioxane at room temperature for 1 h instead of hydrochloric acid in methanol at 90° C. overnight. MS (ESI) calculated for C₂₄H₂₂N₈O: 438.19 m/z, found 439.15 [M+H]⁺.

### Example 107: N-(5-(2-(2-aminopyridin-3-yl)-5-(4-(trifluoromethyl)-1H-pyrazol-1-yl)-3H-imidazo[4,5-b]pyridin-3-yl)-2,3-dihydro-1H-inden-1-yl)-3-formyl-4-hydroxybenzamide

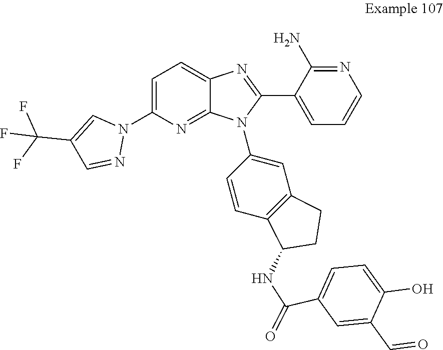 [SMILES: Nc1ncccc1-c1nc2ccc(-n3cc(C(F)(F)F)cn3)nc2n1-c1ccc2c(c1)CC[C@@H]2NC(=O)c1ccc(O)c(C=O)c1]

Example 107 was prepared in a manner analogous to Example 94 using 4-(trifluoromethyl)-1H-pyrazole in place of 4-(difluoromethyl)-1H-pyrazole. MS (ESI) calculated for C₃₂H₂₃F₃N₈O₃: 624.18 m/z, found 625.25 [M+H]⁺. ¹H NMR (400 MHz, DMSO-d₆) δ (ppm): 10.27 (s, 1H), 8.54-8.55 (m, 1H), 8.46-8.48 (m, 1H), 8.26-8.27 (m, 1H), 8.00-8.08 (m, 3H), 7.79-7.80 (m, 1H), 7.45 (m, 1H), 7.32-7.37 (m, 2H), 7.06-7.08 (m, 1H), 7.01-7.02 (m, 1H), 6.80-6.84 (m, 1H), 5.57-5.61 (m, 1H), 3.01-3.07 (m, 1H), 2.85-2.94 (m, 1H), 2.39-2.52 (m, 1H), 2.11-2.14 (m, 1H). ¹⁹F NMR (376 MHz, DMSO-d₆) δ (ppm): −60.89.

### Example 108: N-[(1S)-5-[2-(2-aminopyridin-3-yl)-5-(3-cyanopyrazol-1-yl)imidazo[4,5-b]pyridin-3-yl]-2,3-dihydro-1H-inden-1-yl]-3-formyl-4-hydroxybenzamide

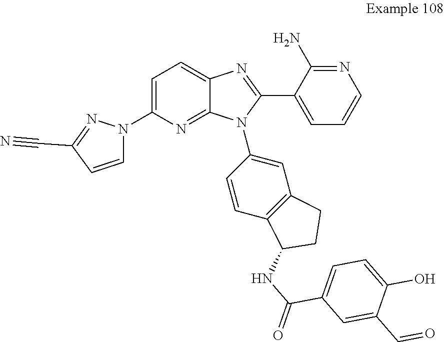 [SMILES: N#Cc1ccn(-c2ccc3nc(-c4cccnc4N)n(-c4ccc5c(c4)CC[C@@H]5NC(=O)c4ccc(O)c(C=O)c4)c3n2)n1]

Example 108 was prepared in a manner analogous to Example 94 using 1H-pyrazole-3-carbonitrile in place of 4-(difluoromethyl)-1H-pyrazole. MS (ESI) calculated for C₃₂H₂₃N₉O₃: 581.19 m/z, found 582.20 [M+H]⁺. ¹H NMR (400 MHz, DMSO-d₆) δ (ppm): 10.31 (s, 1H), 8.62-8.63 (m, 1H), 8.53-8.55 (m, 1H), 8.30-8.31 (m, 1H), 8.06-8.13 (m, 3H), 7.96-7.98 (m, 1H), 7.49 (s, 1H), 7.37 (s, 2H), 7.26-7.27 (m, 1H), 7.03-7.11 (m, 1H), 6.93-6.96 (m, 1H), 5.59-5.61 (m, 1H), 3.01-3.10 (m, 1H), 2.95-3.00 (m, 1H), 2.49-2.50 (m, 1H), 2.08-2.13 (m, 1H).

### Example 109: N-[(1S)-5-[2-(2-aminopyridin-3-yl)-5-(3-fluoropyrazin-2-yl)imidazo[4,5-b]pyridin-3-yl]-2,3-dihydro-1H-inden-1-yl]-3-formyl-4-hydroxybenzamide

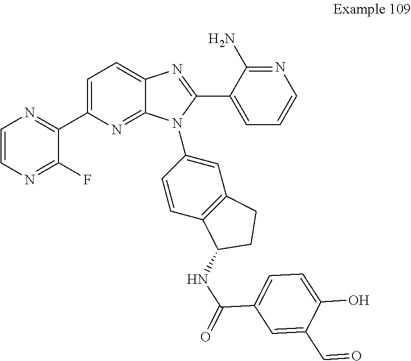 [SMILES: Nc1ncccc1-c1nc2ccc(-c3nccnc3F)nc2n1-c1ccc2c(c1)CC[C@@H]2NC(=O)c1ccc(O)c(C=O)c1]

### Synthetic Route:

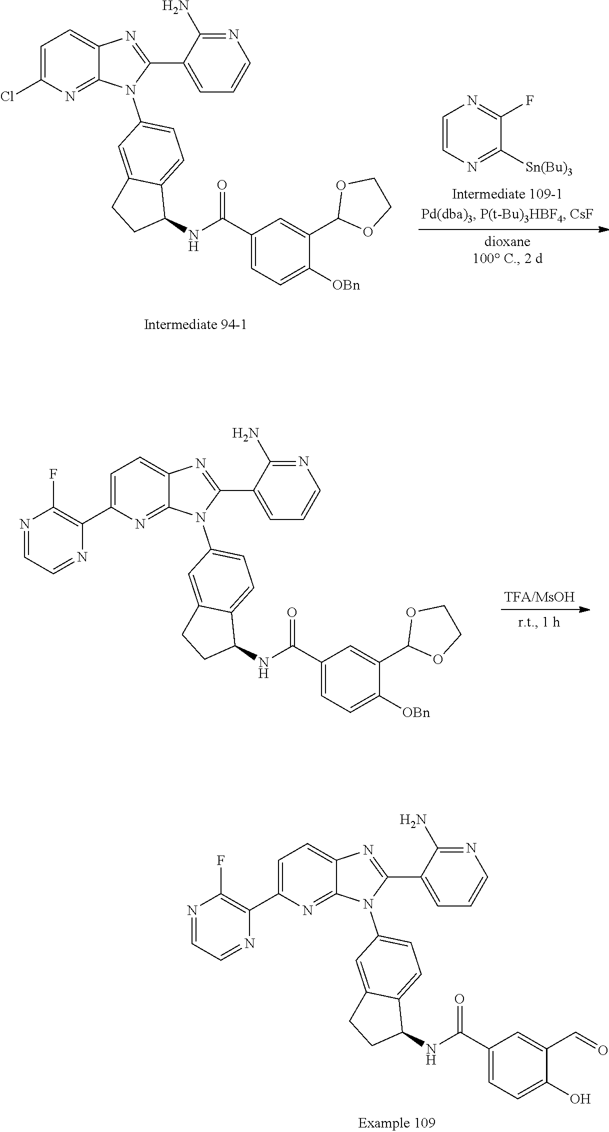

### Step 1: Synthesis of N-[(1S)-5-[2-(2-aminopyridin-3-yl)-5-(3-fluoropyrazin-2-yl)imidazo[4,5-b]pyridin-3-yl]-2,3-dihydro-1H-inden-1-yl]-4-(benzyloxy)-3-(1,3-dioxolan-2-yl)benzamide

To a solution of N-[(1S)-5-[2-(2-aminopyridin-3-yl)-5-chloroimidazo[4,5-b]pyridin-3-yl]-2,3-dihydro-1H-inden-1-yl]-4-(benzyloxy)-3-(1,3-dioxolan-2-yl)benzamide (Intermediate 94-1) (200 mg, 0.303 mmol, 1 equiv) and 2-fluoro-3-(tributylstannyl)pyrazine (Intermediate 109-1) (940 mg, 2.42 mmol, 8 equiv) in 1,4-dioxane (10 mL) was added tris(dibenzylideneacetone)dipalladium(0) (28 mg, 0.030 mmol, 0.1 equiv), tri-tert-butylphosphonium tetrafluoroborate (8.8 mg, 0.030 mmol, 0.1 equiv) and cesium fluoride (115 mg, 0.757 mmol, 2.5 equiv). The resulting mixture was stirred under nitrogen atmosphere at 100° C. for 48 h. The resulting mixture was concentrated under reduced pressure and purified by reverse-phase flash column chromatography on C18 silica gel using a 20-95% gradient of acetonitrile in water (+0.05% ammonium bicarbonate) to afford N-[(1S)-5-[2-(2-aminopyridin-3-yl)-5-(3-fluoropyrazin-2-yl)imidazo[4,5-b]pyridin-3-yl]-2,3-dihydro-1H-inden-1-yl]-4-(benzyloxy)-3-(1,3-dioxolan-2-yl)benzamide (90 mg, 24%) as an off-white solid. MS (ESI) calculated for C₄₁H₃₃FN₈O₄: 720.26 m/z, found 721.30 [M+H]⁺.

### Step 2: Synthesis of N-[(1S)-5-[2-(2-aminopyridin-3-yl)-5-(3-fluoropyrazin-2-yl)imidazo[4,5-b]pyridin-3-yl]-2,3-dihydro-1H-inden-1-yl]-3-formyl-4-hydroxybenzamide (Example 109)

To a solution of N-[(1S)-5-[2-(2-aminopyridin-3-yl)-5-(3-fluoropyrazin-2-yl)imidazo[4,5-b]pyridin-3-yl]-2,3-dihydro-1H-inden-1-yl]-4-(benzyloxy)-3-(1,3-dioxolan-2-yl)benzamide (90 mg, 0.13 mmol, 1 equiv) in 2,2,2-trifluoroacetic acid (1.5 mL) was added methanesulfonic acid (0.5 mL). The resulting mixture was stirred at room temperature for 1 h then concentrated in vacuo and purified by Preparative HPLC on a YMC EXSA C18 Column using a 30-90% gradient of acetonitrile in water (+10 mmol/L ammonium bicarbonate) to afford N-[(1S)-5-[2-(2-aminopyridin-3-yl)-5-(3-fluoropyrazin-2-yl)imidazo[4,5-b]pyridin-3-yl]-2,3-dihydro-1H-inden-1-yl]-3-formyl-4-hydroxybenzamide (10.6 mg, 14%) as a light yellow solid. MS (ESI) calculated for C₃₂H₂₃FN₈O₃: 586.19 m/z, found 587.25 [M+H]⁺. ¹H NMR (400 MHz, DMSO-d₆) δ (ppm): 10.27 (s, 1H), 8.72-8.74 (m, 1H), 8.36-8.39 (m, 2H), 8.11-8.26 (m, 1H), 8.04-8.09 (m, 1H), 8.01-8.03 (m, 2H), 7.40 (s, 1H), 7.30-7.32 (m, 2H), 7.25-7.27 (m, 1H), 6.99-7.01 (m, 1H), 6.45-6.49 (m, 1H), 5.58-5.62 (m, 1H), 2.98-2.99 (m, 1H), 2.87-2.89 (m, 1H), 2.50-2.52 (m, 1H), 2.07-2.08 (m, 1H). ¹⁹F NMR (376 MHz, DMSO-d₆) δ (ppm): −74.57.

### Intermediate 109-1: 2-fluoro-3-(tributylstannyl)pyrazine

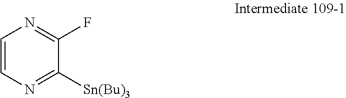

### Synthetic Route:

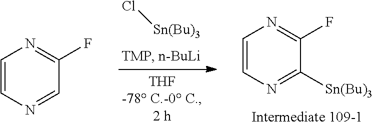

### Step 1: Synthesis of 2-fluoro-3-(tributylstannyl)pyrazine (Intermediate 109-1)

To a cooled (−78° C.) solution of 2,2,6,6-tetramethylpiperidine (1.73 g, 12.2 mmol, 1.2 equiv) in tetrahydrofuran (20 mL) was added dropwise n-butyllithium (2.5M in hexanes, 4.49 mL, 11.2 mmol, 1.1 equiv). The resulting mixture was stirred at 0° C. for 20 minutes and then cooled to −78° C. A solution of 2-fluoropyrazine (1.00 g, 10.2 mmol, 1 equiv) in tetrahydrofuran (5 mL) was added dropwise. After 5 minutes, tributylchlorostannane (3.65 g, 11.2 mmol, 1.1 equiv) was added and the reaction mixture was stirred at −78° C. for 1 h. The reaction was quenched with saturated aqueous ammonium chloride (30 mL) and extracted with ethyl acetate (30 mL×3). The combined extracts were washed with brine (30 mL), dried over anhydrous sodium sulfate, filtered, and concentrated in vacuo. The resulting oil was then purified by silica gel column chromatography using a 0-25% gradient of ethyl acetate in petroleum ether to afford 2-fluoro-3-(tributylstannyl) pyrazine (Intermediate 109-1) (2.5 g, 63%) as a colorless oil. MS (ESI) calculated for C₁₆H₂₉FN₂Sn: 388.13 m/z, found 389.05 [M+H]⁺.

### Example 110: N-[(1S)-5-[2-(2-aminopyridin-3-yl)-5-(pyrazol-1-yl)imidazo[4,5-b]pyridin-3-yl]-2,3-dihydro-1H-inden-1-yl]-2-chloro-3-fluoro-5-formyl-4-hydroxybenzamide

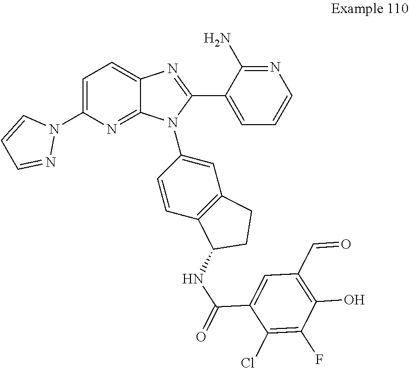 [SMILES: Nc1ncccc1-c1nc2ccc(-n3cccn3)nc2n1-c1ccc2c(c1)CC[C@@H]2NC(=O)c1cc(C=O)c(O)c(F)c1Cl]

Example 110 was prepared in a manner analogous to Example 12 using Intermediate 33-1 in place of Intermediate 1-3 and Intermediate 110-1 in place of Intermediate 12-1. MS (ESI) calculated for C₃₁H₂₂ClFN₈O₃: 608.15 m/z, found 609.15 [M+H]⁺. ¹H NMR (400 MHz, DMSO-d₆) δ (ppm): 10.26 (s, 1H), 9.06-9.08 (m, 1H), 8.41-8.44 (m, 1H), 8.37-8.38 (m, 1H), 8.00-8.07 (m, 1H), 7.82-7.83 (m, 1H), 7.63-7.68 (m, 1H), 7.46-7.49 (m, 1H), 7.36-7.38 (m, 2H), 7.35-7.36 (m, 1H), 6.74-6.75 (m, 1H), 6.56-6.57 (m, 1H), 5.21-5.55 (m, 1H), 2.94-3.06 (m, 1H), 2.90-2.92 (m, 1H), 2.56-2.57 (m, 1H), 2.02-2.05 (m, 1H).

### Intermediate 110-1: 4-(benzyloxy)-2-chloro-5-(1,3-dioxolan-2-yl)-3-fluorobenzoic acid

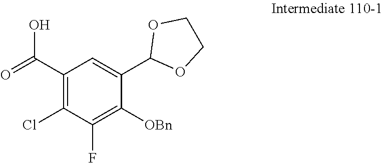 [SMILES: O=C(O)c1cc(C2OCCO2)c(OCc2ccccc2)c(F)c1Cl]

### Synthetic Route:

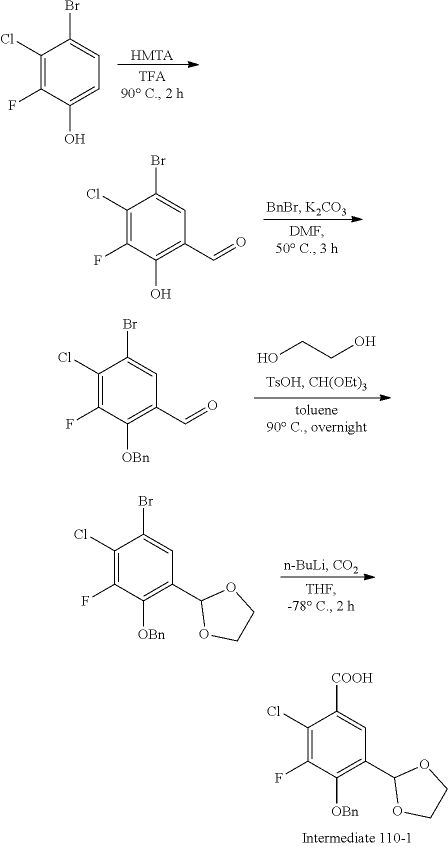

### Step 1: Synthesis of 5-bromo-4-chloro-3-fluoro-2-hydroxybenzaldehyde

A solution of 4-bromo-3-chloro-2-fluorophenol (3 g, 13.307 mmol, 1 equiv) and 1,3,5,7-tetraazaadamantane (9.33 g, 66.5 mmol, 5 equiv) in 2,2,2-trifluoroacetic acid (50 mL) was stirred at 90° C. for 10 h. The resulting mixture was neutralized with 2M aqueous sodium hydroxide. The precipitated solids were collected by filtration, washed with water, and purified by reverse-phase column chromatography on C18 silica gel using a 10-95% gradient of acetonitrile in water to provide 5-bromo-4-chloro-3-fluoro-2-hydroxybenzaldehyde (1.7 g, 45%) as a yellow solid. MS (ESI) calculated for C₇H₃BrClFO₂: 251.90 m/z, found 252.95 [M+H]⁺.

### Step 2: Synthesis of 2-(benzyloxy)-5-bromo-4-chloro-3-fluorobenzaldehyde

A mixture of 5-bromo-4-chloro-3-fluoro-2-hydroxybenzaldehyde (1.7 g, 6.7 mmol, 1 equiv), benzyl bromide (3.44 g, 20.1 mmol, 3 equiv) and cesium carbonate (4.37 g, 13.4 mmol, 2 equiv) in acetonitrile (50 mL) was stirred at room temperature for 2 h. The resulting mixture was filtered and concentrated in vacuo. The residue was taken up into water and ethyl acetate. The layers were separated, and the aqueous phase was extracted twice with ethyl acetate. The combined organic layers were washed with brine, dried over anhydrous sodium sulfate, filtered, and concentrated under reduced pressure. The crude residue was purified by silica gel column chromatography using a gradient of ethyl acetate in petroleum ether to provide 2-(benzyloxy)-5-bromo-4-chloro-3-fluorobenzaldehyde (1.9 g, 78%) as a white solid. MS (ESI) calculated for C₁₄H₉BrClFO₂: 341.95 m/z, found 342.90 [M+H]⁺.

### Step 3: Synthesis of 2-[2-(benzyloxy)-5-bromo-4-chloro-3-fluorophenyl]-1,3-dioxolane

A mixture of 2-(benzyloxy)-5-bromo-4-chloro-3-fluorobenzaldehyde (1.9 g, 5.5 mmol, 1 equiv), ethylene glycol (1.72 g, 27.7 mmol, 5 equiv), p-toluenesulfonic acid (0.19 g, 1.1 mmol, 0.2 equiv) and triethyl orthoformate (2.46 g, 16.6 mmol, 3 equiv) in toluene (40 mL) was stirred at 100° C. overnight. The reaction mixture was cooled to 0° C. and quenched with water. The resulting mixture was extracted with ethyl acetate (100 mL×3). The combined organic layers were washed with brine, dried over anhydrous sodium sulfate, filtered, and concentrated under reduced pressure to provide 2-[2-(benzyloxy)-5-bromo-4-chloro-3-fluorophenyl]-1,3-dioxolane (2.2 g, 83% crude) as a yellow green solid, which was used in the next step without purification. MS (ESI) calculated for C₁₆H₁₃BrClFO₃: 385.27 m/z, found 384.90 [M−H]⁻.

### Step 4: Synthesis of 4-(benzyloxy)-2-chloro-5-(1,3-dioxolan-2-yl)-3-fluorobenzoic acid (Intermediate 110-1)

To a cooled (−78° C.) solution of 2-[2-(benzyloxy)-5-bromo-4-chloro-3-fluorophenyl]-1,3-dioxolane (700 mg, 1.81 mmol, 1 equiv) in tetrahydrofuran (20 mL) was added n-butyllithium (2.5 M in hexanes, 1.8 mL, 4.5 mmol, 2.5 equiv) and the resulting mixture was stirred for 1 h under nitrogen atmosphere then for 2 h under carbon dioxide. The reaction was quenched with saturated aqueous ammonium chloride (10 mL) and extracted 3 times with ethyl acetate. The combined organic layers were washed with brine, dried over anhydrous sodium sulfate, filtered, and concentrated in vacuo. The resulting residue was purified by reverse-phase flash column chromatography on C18 silica gel using a 0-45% gradient of acetonitrile in water (+0.05% ammonium bicarbonate) to afford 4-(benzyloxy)-2-chloro-5-(1,3-dioxolan-2-yl)-3-fluorobenzoic acid (Intermediate 110-1) (670 mg, 84%) as a white solid. MS (ESI) calculated for C₁₇H₁₄ClFO₅: 352.05 m/z, found 351.05 [M−H]⁻.

### Example 111: N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-6-methanesulfonamidopyridine-3-carboxamide

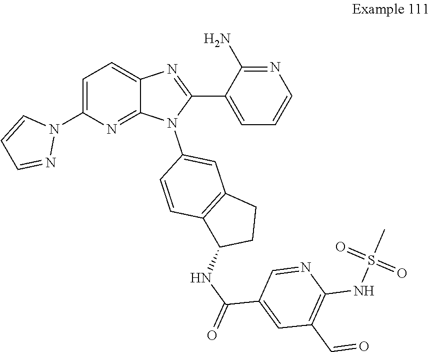 [SMILES: CS(=O)(=O)Nc1ncc(C(=O)N[C@H]2CCc3cc(-n4c(-c5cccnc5N)nc5ccc(-n6cccn6)nc54)ccc32)cc1C=O]

Example 111 was prepared in a manner analogous to Example 12 using Intermediate 33-1 in place of Intermediate 1-3 and Intermediate 111-1 in place of Intermediate 12-1. MS (ESI) calculated for C₃₁H₂₆N₁₀O₄S: 634.19 m/z, found 635.20 [M+H]⁺. ¹H NMR (400 MHz, DMSO-d₆) δ (ppm): 10.06 (s, 1H), 8.49-8.50 (m, 1H), 8.32-8.39 (m, 3H), 8.02-8.03 (m, 1H), 7.97-7.99 (m, 1H), 7.81-7.82 (m, 1H), 7.51-7.54 (m, 1H), 7.39 (s, 1H), 7.24-7.29 (m, 3H), 6.62-6.66 (m, 1H), 6.55-6.56 (m, 1H), 5.26-5.30 (m, 1H), 3.60-3.66 (m, 3H), 2.92-2.96 (m, 1H), 2.82-2.86 (m, 1H), 2.40-2.45 (m, 1H), 1.82-1.85 (m, 1H).

### Intermediate 111-1: 5-(1,3-dioxolan-2-yl)-6-(methylsulfonamido)nicotinic acid

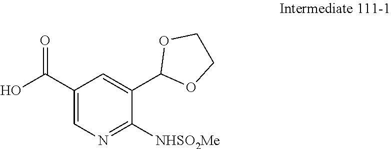

### Synthetic Route:

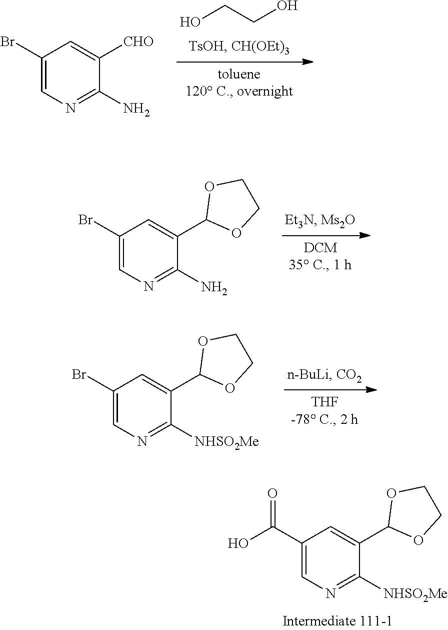

### Step 1: Synthesis of 5-bromo-3-(1,3-dioxolan-2-yl)pyridin-2-amine

A solution of 2-amino-5-bromopyridine-3-carbaldehyde (2.00 g, 9.95 mmol, 1 equiv), ethylene glycol (3.09 g, 49.7 mmol, 5 equiv), triethyl orthoformate (4.42 g, 29.9 mmol, 3 equiv) and p-toluenesulfonic acid (2.06 g, 11.9 mmol, 1.2 equiv) in toluene (50 mL) was stirred for overnight at 120° C. The resulting mixture was concentrated under reduced pressure and taken up into chloroform/isopropanol (3:1) (500 mL). The solution was washed with water (500 mL×3), dried over anhydrous sodium sulfate, filtered, and concentrated under reduced pressure. The residue was purified by silica gel column chromatography using a 0-25% gradient of ethyl acetate in petroleum ether to afford 5-bromo-3-(1,3-dioxolan-2-yl)pyridin-2-amine (1 g, 37%) as a yellow solid. MS (ESI) calculated for C₈H₉BrN₂O₂: 243.98 m/z, found 244.95 [M+H]⁺.

### Step 2: Synthesis of N-[5-bromo-3-(1,3-dioxolan-2-yl)pyridin-2-yl]methanesulfonamide

A solution of 5-bromo-3-(1,3-dioxolan-2-yl)pyridin-2-amine (1.00 g, 4.08 mmol, 1 equiv), methanesulfonic anhydride (2.13 g, 12.2 mmol, 3 equiv) and triethylamine (1.24 g, 12.2 mmol, 3 equiv) in dichloromethane (30 mL) was stirred for 1 h at 35° C. The mixture was concentrated under reduced pressure and the crude product was purified by reverse-phase flash column chromatography on C18 silica gel using a 5-95% gradient of acetonitrile in water (+0.05% ammonium bicarbonate) to afford N-[5-bromo-3-(1,3-dioxolan-2-yl)pyridin-2-yl]methanesulfonamide (450 mg, 29%). MS (ESI) calculated for C₉H₁₁BrN₂O₄S: 321.96 m/z, found 322.90 [M+H]⁺.

### Step 3: Synthesis of 5-(1,3-dioxolan-2-yl)-6-methanesulfonamidopyridine-3-carboxylic acid (Intermediate 111-1)

To a cooled (−78° C.) solution of N-[5-bromo-3-(1,3-dioxolan-2-yl)pyridin-2-yl]methanesulfonamide (450 mg, 1.39 mmol, 1 equiv) in tetrahydrofuran (15 mL) was added n-butyllithium (2.5 M in hexanes, 1.4 mL, 3.48 mmol, 2.5 equiv) and the resulting mixture was stirred at −78° C. for 1 h under nitrogen atmosphere followed by 1 h under carbon dioxide. The resulting mixture was concentrated under reduced pressure and the crude product was purified by reverse-phase flash column chromatography on C18 silica gel using a 0-95% gradient of acetonitrile in water (+0.05% ammonium bicarbonate) to afford 5-(1,3-dioxolan-2-yl)-6-methanesulfonamidopyridine-3-carboxylic acid (Intermediate 111-1) (150 mg, 32%). MS (ESI) calculated for C₁₀H₁₂N₂O₆S: 288.04 m/z, found 289.00 [M+H]⁺.

### Example 112: N-[(1S)-5-[2-(2-aminopyridin-3-yl)-5-(pyrazol-1-yl)imidazo[4,5-b]pyridin-3-yl]-2,3-dihydro-1H-inden-1-yl]-2-ethynyl-3-formyl-4-hydroxybenzamide

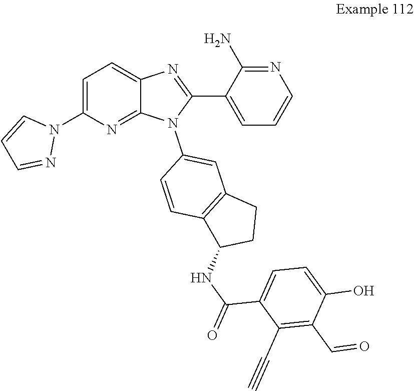 [SMILES: C#Cc1c(C(=O)N[C@H]2CCc3cc(-n4c(-c5cccnc5N)nc5ccc(-n6cccn6)nc54)ccc32)ccc(O)c1C=O]

Example 112 was prepared in a manner analogous to Example 12 using Intermediate 33-1 in place of Intermediate 1-3 and Intermediate 112-1 in place of Intermediate 12-1. MS (ESI) calculated for C₃₃H₂₄N₈O₃: 580.20 m/z, found 581.25 [M+H]⁺. ¹H NMR (400 MHz, DMSO-d₆) δ (ppm): 10.38 (s, 1H), 8.33-8.35 (m, 2H), 7.98-8.00 (m, 1H), 7.93-7.95 (m, 1H), 7.79 (s, 1H), 7.51-7.39 (m, 2H), 7.37 (s, 1H), 7.27-7.26 (m, 2H), 6.99-7.00 (m, 1H), 6.54 (s, 1H), 6.45-6.36 (m, 1H), 5.51-5.54 (m, 1H), 4.63 (m, 1H), 2.97-2.98 (m, 1H), 2.88-2.90 (m, 1H), 2.51-2.53 (m, 1H), 2.01-2.04 (m, 1H).

### Intermediate 112-1: 4-(benzyloxy)-3-(1,3-dioxolan-2-yl)-2-ethynylbenzoic acid

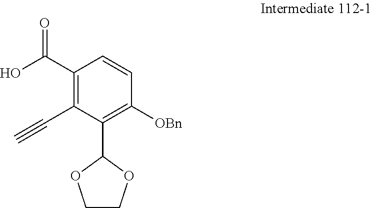 [SMILES: C#Cc1c(C(=O)O)ccc(OCc2ccccc2)c1C1OCCO1]

### Synthetic Route:

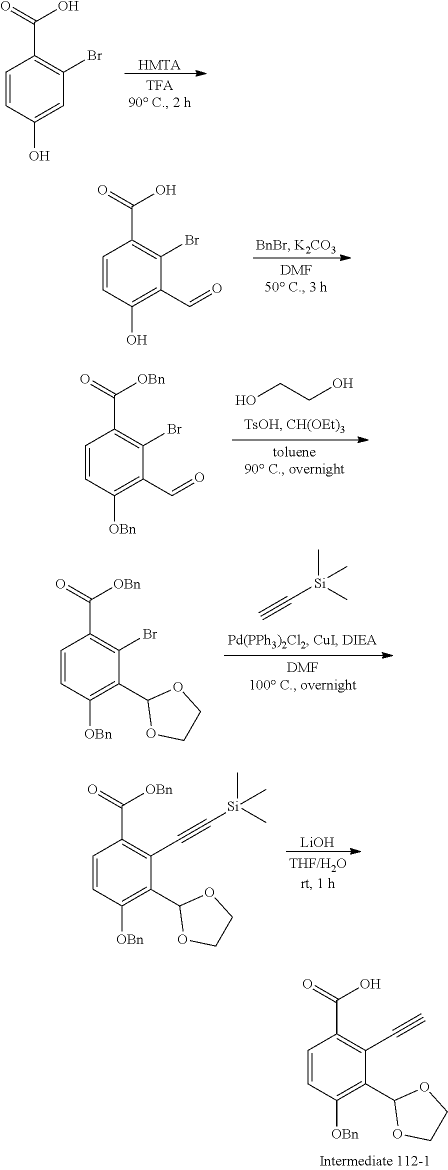

### Step 1: Synthesis of 2-bromo-3-formyl-4-hydroxybenzoic acid

A mixture of 2-bromo-4-hydroxybenzoic acid (1.60 g, 7.37 mmol, 1 equiv) and 1,3,5,7-tetraazaadamantane (1.55 g, 11.1 mmol, 1.5 equiv) in 2,2,2-trifluoroacetic acid (16 mL) was stirred at 90° C. for 2 h. The resulting mixture was concentrated under reduced pressure and taken up into ethyl acetate (30 mL) and water (30 mL). The layers were separated, and the aqueous phase was extracted with ethyl acetate (40 mL×2). The combined organic layers were washed with brine (80 mL×2), dried over anhydrous sodium sulfate, filtered, and concentrated in vacuo. The resulting residue was purified by reverse phase flash column chromatography on C18 silica gel using a gradient of acetonitrile in water (+0.05% ammonium bicarbonate) to afford 2-bromo-3-formyl-4-hydroxybenzoic acid (560 mg, 31%) as a light-yellow solid. MS (ESI) calculated for C₈H₅BrO₄: 243.94 m/z, found 244.95 [M−H]⁺.

### Step 2: Synthesis of benzyl 4-(benzyloxy)-2-bromo-3-formylbenzoate

To a stirred mixture of 2-bromo-3-formyl-4-hydroxybenzoic acid (0.56 g, 2.3 mmol, 1 equiv) and potassium carbonate (1.26 g, 9.14 mmol, 4 equiv) in N,N-dimethylformamide (10 mL) was added benzyl bromide (1.56 g, 9.140 mmol, 4 equiv) and the resulting mixture was stirred at room temperature for 2 h. Ethyl acetate (50 mL) and water (50 mL) were added. The layers were separated, and the aqueous phase was extracted with ethyl acetate (100 mL×2). The combined organic layers were washed with brine (300 mL×3), dried over anhydrous sodium sulfate, filtered and concentrated in vacuo. The residue was purified by silica gel column chromatography eluting with 25% ethyl acetate in petroleum ether to afford benzyl 4-(benzyloxy)-2-bromo-3-formylbenzoate (0.61 g, 63%) as a white solid. MS (ESI) calculated for C₂₂H₁₇BrO₄: 424.03 m/z, found 425.00 [M+H]⁺.

### Step 3: Synthesis of benzyl 4-(benzyloxy)-2-bromo-3-(1,3-dioxolan-2-yl)benzoate

To a solution of benzyl 4-(benzyloxy)-2-bromo-3-formylbenzoate (1.00 g, 2.35 mmol, 1 equiv) in toluene (20 mL) was added ethylene glycol (0.73 g, 12 mmol, 5 equiv), triethyl orthoformate (1.05 g, 7.05 mmol, 3 equiv) and p-toluenesulfonic acid (0.040 g, 0.24 mmol, 0.1 equiv). The obtained solution was stirred at room temperature for 10 min then overnight at 90° C. The reaction mixture was cooled to 0° C. and quenched by the addition of saturated aqueous sodium bicarbonate (20 mL). The resulting mixture was extracted with ethyl acetate (50 mL×3). The combined organic layers were washed with water (50 mL×3), dried over anhydrous sodium sulfate, filtered, and concentrated under reduced pressure. The residue was purified by silica gel column chromatography using a 0-17% gradient of ethyl acetate in petroleum ether to provide benzyl 4-(benzyloxy)-2-bromo-3-(1,3-dioxolan-2-yl)benzoate (1 g, 91%) as a colorless oil. MS (ESI) calculated for C₂₄H₂₁BrO₅: 468.06 m/z, found 469.20 [M+H]⁺.

### Step 4: Synthesis of benzyl 4-(benzyloxy)-3-(1,3-dioxolan-2-yl)-2-[2-(trimethylsilyl)ethynyl]benzoate

To a solution of benzyl 4-(benzyloxy)-2-bromo-3-(1,3-dioxolan-2-yl)benzoate (1.00 g, 2.13 mmol, 1 equiv) in N,N-dimethylformamide (10 mL) was added trimethylsilylacetylene (1.67 g, 17 mmol, 8 equiv), bis(triphenylphosphine)palladium(II) dichloride (0.15 g, 0.21 mmol, 0.1 equiv), copper (I) iodide (0.040 g, 0.21 mmol, 0.1 equiv) and N,N-diisopropylethylamine (0.55 g, 4.3 mmol, 2 equiv). The resulting mixture was stirred at 80° C. for 2 h under nitrogen atmosphere. The reaction mixture was cooled to 0° C. and quenched by the addition of saturated aqueous ammonium chloride. The mixture was extracted with ethyl acetate (10 mL×3). The combined organic layers were washed with water (10 mL×3), dried over anhydrous sodium sulfate, filtered, and concentrated under reduced pressure. The residue was purified by silica gel column chromatography using a 0-17% gradient of ethyl acetate in petroleum ether to provide benzyl 4-(benzyloxy)-3-(1,3-dioxolan-2-yl)-2-[2-(trimethylsilyl)ethynyl]benzoate (50 mg, 5%) as a light brown oil. MS (ESI) calculated for C₂₉H₃₀O₅Si: 486.19 m/z, found 487.20 [M+H]⁺.

### Step 5: Synthesis of 4-(benzyloxy)-3-(1,3-dioxolan-2-yl)-2-ethynylbenzoic acid (Intermediate 112-1)

To a cooled (0° C.) solution of benzyl 4-(benzyloxy)-3-(1,3-dioxolan-2-yl)-2-[2-(trimethylsilyl)ethynyl]benzoate (80 mg, 0.16 mmol, 1 equiv) in tetrahydrofuran (10 mL) was added a solution of lithium hydroxide (16 mg, 0.66 mmol, 4 equiv) in water (3 mL). The obtained solution was stirred for 2 h at room temperature then concentrated under reduced pressure. The obtained solution was acidified to pH 6 with 2N hydrochloric acid. The resulting precipitate was collected by filtration to provide 4-(benzyloxy)-3-(1,3-dioxolan-2-yl)-2-ethynylbenzoic acid (Intermediate 112-1) (50 mg, 94%) as a brown solid. MS (ESI) calculated for C₁₉H₁₆O₅: 324.10 m/z, found 325.20 [M+H]⁺.

### Example 113: N-[(1S)-5-[2-(2-aminopyridin-3-yl)-5-[1-(difluoromethyl) pyrazol3-yl]imidazo [4,5-b] pyridin-3-yl]-2,3-dihydro-1H-inden-1-yl]-3-formyl-4-hydroxybenzamide

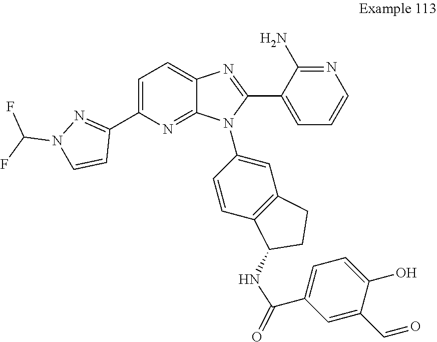 [SMILES: Nc1ncccc1-c1nc2ccc(-c3ccn(C(F)F)n3)nc2n1-c1ccc2c(c1)CC[C@@H]2NC(=O)c1ccc(O)c(C=O)c1]

### Synthetic Route:

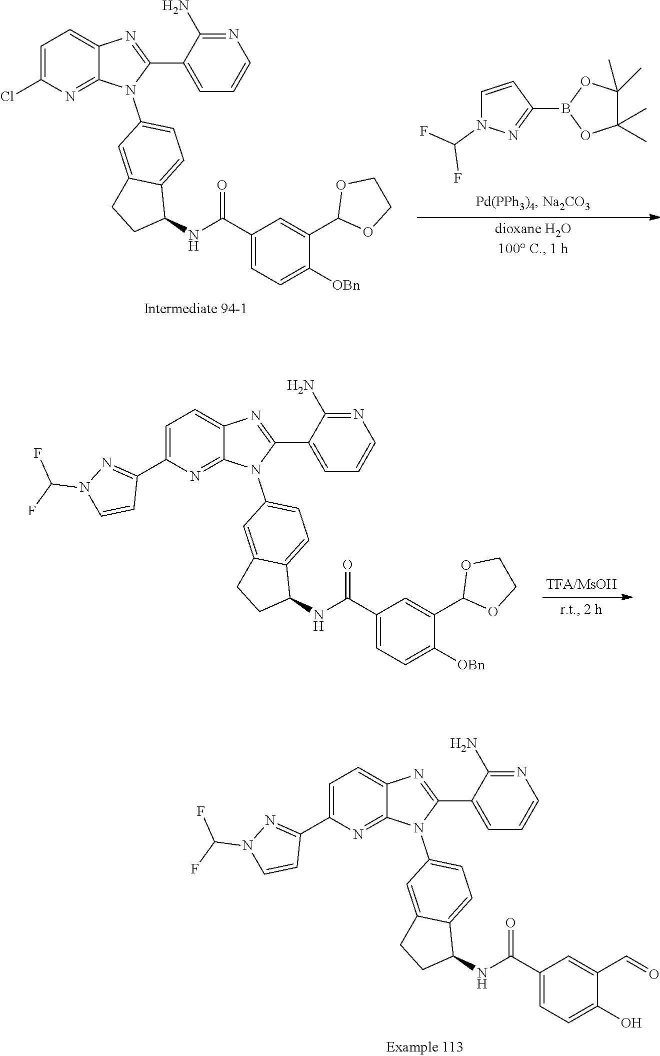 [SMILES: Nc1ncccc1-c1nc2ccc(Cl)nc2n1-c1ccc2c(c1)CC[C@@H]2NC(=O)c1ccc(OCc2ccccc2)c(C2OCCO2)c1 ; CC1(C)OB(c2ccn(C(F)F)n2)OC1(C)C ; Nc1ncccc1-c1nc2ccc(-c3ccn(C(F)F)n3)nc2n1-c1ccc2c(c1)CC[C@@H]2NC(=O)c1ccc(OCc2ccccc2)c(C2OCCO2)c1 ; Nc1ncccc1-c1nc2ccc(-c3ccn(C(F)F)n3)nc2n1-c1ccc2c(c1)CC[C@@H]2NC(=O)c1ccc(O)c(C=O)c1]

### Step 1: Synthesis of N-[(1S)-5-[2-(2-aminopyridin-3-yl)-5-[1-(difluoromethyl) pyrazol-3-yl] imidazo[4,5-b] pyridin-3-yl]-2,3-dihydro-1H-inden-1-yl]-4-(benzyloxy)-3-(1,3-dioxolan-2-yl) benzamide

A mixture of N-[(1S)-5-[2-(2-aminopyridin-3-yl)-5-chloroimidazo[4,5-b]pyridin-3-yl]-2,3-dihydro-1H-inden-1-yl]-4-(benzyloxy)-3-(1,3-dioxolan-2-yl)benzamide (Intermediate 94-1) (300 mg, 0.455 mmol, 1 equiv), 1-(difluoromethyl)-3-(4,4,5,5-tetramethyl-1,3,2-dioxaborolan-2-yl)pyrazole (333 mg, 1.37 mmol, 3 equiv), sodium carbonate (146 mg, 1.37 mmol, 3 equiv) and tetrakis(triphenylphosphine)palladium(0) (105 mg, 0.091 mmol, 0.2 equiv) in water (2 mL) and 1,4-dioxane (8 mL) was stirred for 1 h at 100° C. under nitrogen atmosphere. The resulting mixture was concentrated under reduced pressure and the residue obtained was purified by reverse-phase flash column chromatography using a gradient of acetonitrile in water (+0.05% ammonium bicarbonate) to afford N-[(1S)-5-[2-(2-aminopyridin-3-yl)-5-[1-(difluoromethyl) pyrazol-3-yl]imidazo[4,5-b]pyridin-3-yl]-2,3-dihydro-1H-inden-1-yl]-4-(benzyloxy)-3-(1,3-dioxolan-2-yl) benzamide (350 mg, quant) as a yellow oil. MS (ESI) calculated for C₄₁H₃₄F₂N₈O₄: 740.27 m/z, found 741.30 [M+H]⁺.

### Step 2: Synthesis of N-[(1S)-5-[2-(2-aminopyridin-3-yl)-5-[1-(difluoromethyl) pyrazol-3-yl] imidazo[4,5-b]pyridine-3-yl]-2,3-dihydro-1H-inden-1-yl]-3-formyl-4-hydroxybenzamide (Example 113)

To a stirred solution of N-[(1S)-5-[2-(2-aminopyridin-3-yl)-5-[1-(difluoromethyl) pyrazol-3-yl]imidazo[4,5-b]pyridin-3-yl]-2,3-dihydro-1H-inden-1-yl]-4-(benzyloxy)-3-(1,3-dioxolan-2-yl) benzamide (200 mg, 0.270 mmol, 1 equiv) in 2,2,2-trifluoroacetic acid (1.5 mL) was added methanesulfonic acid (0.3 mL) and the resulting mixture was stirred for 2 h at room temperature. The residue was purified by Preparative HPLC on a XBridge Prep OBD C18 Column using a 24-42% gradient of acetonitrile in water (+10 mmol/L ammonium bicarbonate) to afford N-[(1S)-5-[2-(2-aminopyridin-3-yl)-5-[1-(difluoromethyl) pyrazol-3-yl] imidazo[4,5-b]pyridin-3-yl]-2,3-dihydro-1H-inden-1-yl]-3-formyl-4-hydroxybenzamide (Example 113) (34.0 mg, 20%) as an off-white solid. MS (ESI) calculated for C₃₂H₂₄F₂N₈O₃: 606.19 m/z, found 607.20 [M+H]⁺. ¹H NMR (300 MHz, DMSO-d₆) δ (ppm): 10.83-11.52 (m, 1H), 10.08-10.42 (m, 1H), 8.63-9.12 (m, 1H), 8.19-8.35 (m, 3H), 7.61-8.17 (m, 4H), 7.15-7.51 (m, 4H), 7.01-7.11 (m, 1H), 6.71-7.01 (m, 3H), 6.15-6.55 (m, 1H), 5.35-5.82 (m, 1H), 2.85-3.11 (m, 2H), 2.35-2.49 (m, 1H), 1.88-2.15 (m, 1H).

### Example 114: (S)—N-(5-(2-(2-aminopyridin-3-yl)-5-(3-(dimethylamino)-1H-pyrazol-1-yl)-3H-imidazo[4,5-b]pyridin-3-yl)-2,3-dihydro-1H-inden-1-yl)-3-formyl-4-hydroxybenzamide

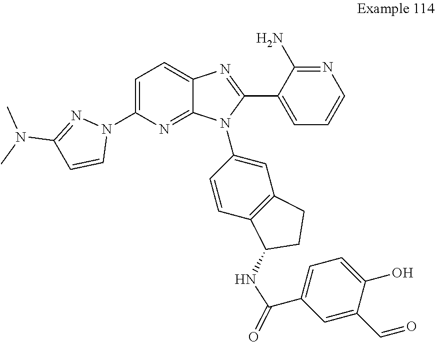 [SMILES: CN(C)c1ccn(-c2ccc3nc(-c4cccnc4N)n(-c4ccc5c(c4)CC[C@@H]5NC(=O)c4ccc(O)c(C=O)c4)c3n2)n1]

Example 114 was prepared in a manner analogous to Example 94 using Intermediate 114-1 in place of 4-(difluoromethyl)-1H-pyrazole. MS (ESI) calculated for C₃₃H₂₉N₉O₃: 599.24 m/z, found 600.25 [M+H]⁺. ¹H NMR (400 MHz, DMSO-d₆) δ (ppm): 10.28 (s, 1H), 8.20-8.30 (m, 2H), 7.93-8.15 (m, 3H), 7.69-7.80 (m, 1H), 7.30-7.40 (m, 2H), 7.19-7.28 (m, 2H), 6.90-7.10 (m, 1H), 6.38-4.49 (m, 1H), 6.00-6.10 (m, 1H), 5.50-5.65 (m, 1H), 2.95-3.10 (m, 1H), 2.80-2.95 (m, 7H), 2.40-2.50 (m, 1H), 2.00-2.18 (m, 1H).

### Intermediate 114-1: N,N-dimethyl-1H-pyrazol-3-amine

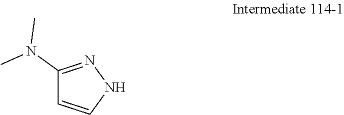

### Synthetic Route:

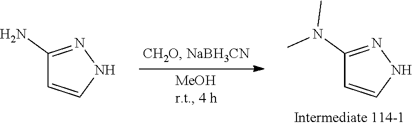

### Step 1: Synthesis of N, N-dimethyl-1H-pyrazol-3-amine (Intermediate 114-1)

To a solution of 1H-pyrazol-3-amine (1.0 g, 12 mmol, 1 equiv) in methanol (30 mL) were added formaldehyde (aqueous) (0.2 mL, 2.4 mmol, 4 equiv) and sodium cyanoborohydride (1.89 g, 30.1 mmol, 2.5 equiv). The resulting solution was stirred at room temperature for 4 h. The reaction mixture was concentrated in vacuo and purified by reverse-phase flash column chromatography on C18 silica gel using a 0-30% gradient of acetonitrile in water (+0.05% ammonium bicarbonate) to afford N,N-dimethyl-1H-pyrazol-3-amine (600 mg, 45%) as a colorless oil. MS (ESI) calculated for C₅H₉N₃: 111.08 m/z, found 112.15 [M+H]⁺.

### Example 115: N-[(1S)-5-[2-(2-aminopyridin-3-yl)-5-(1,2,3-triazol-2-yl)imidazo[4,5-b]pyridin-3-yl]-2,3-dihydro-1H-inden-1-yl]-3-fluoro-5-formyl-4-hydroxybenzamide

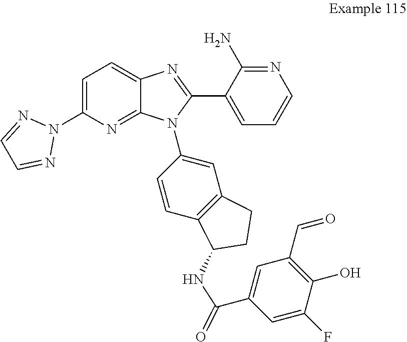 [SMILES: Nc1ncccc1-c1nc2ccc(-n3nccn3)nc2n1-c1ccc2c(c1)CC[C@@H]2NC(=O)c1cc(F)c(O)c(C=O)c1]

Example 115 was prepared in a manner analogous to Example 12 using Intermediate 91-2 in place of Intermediate 1-3 and Intermediate 44-1 in place of Intermediate 12-1. MS (ESI) calculated for C₃₀H₂₂FN₉O₃: 575.18 m/z, found 576.20 [M+H]⁺. ¹H NMR (400 MHz, DMSO-d₆) δ (ppm): 10.22 (s, 1H), 8.39-8.41 (m, 1H), 7.98-8.09 (m, 5H), 7.82-8.85 (m, 1H), 7.27-7.35 (m, 4H), 6.45-7.48 (m, 1H), 5.56-5.60 (m, 1H), 2.86-2.99 (m, 2H), 2.51 (s, 1H), 2.03-2.08 (m, 1H). ¹⁹F-NMR (400 MHz, DMSO-d₆) δ (ppm): −134.70.

### Example 116: (S)—N-(5-(2-(2-aminopyridin-3-yl)-5-(3-(difluoromethyl)-1H-pyrazol-1-yl)-3H-imidazo[4,5-b]pyridin-3-yl)-2,3-dihydro-1H-inden-1-yl)-3-formyl-4-hydroxybenzamide

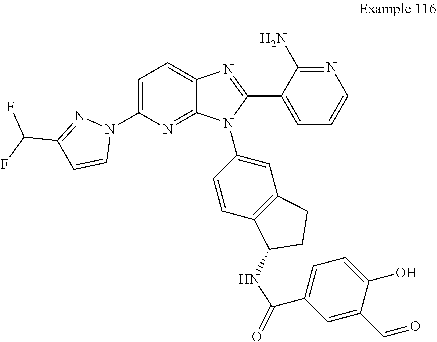 [SMILES: Nc1ncccc1-c1nc2ccc(-n3ccc(C(F)F)n3)nc2n1-c1ccc2c(c1)CC[C@@H]2NC(=O)c1ccc(O)c(C=O)c1]

Example 116 was prepared in as manner analogous to Example 94 using Intermediate 116-1 in place of 4-(difluoromethyl)-1H-pyrazole. MS (ESI) calculated for C₃₂H₂₄F₂N₈O₃: 606.19 m/z, found 607.20 [M+H]⁺. ¹H NMR (400 MHz, DMSO-d₆) δ (ppm): 10.28 (s, 1H), 8.39-8.49 (m, 2H), 8.26-8.27 (m, 1H), 8.01-8.04 (m, 3H), 7.27-7.38 (m, 4H), 6.95-7.01 (m, 2H), 6.81-6.82 (m, 1H), 6.45-6.55 (m, 1H), 5.60-5.64 (m, 1H), 2.85-3.05 (m, 2H), 2.46-2.51 (m, 1H), 2.07-2.33 (m, 1H). ¹⁹F NMR (400 MHz, DMSO-d₆) δ (ppm): −122.03.

### Intermediate 116-1: 3-(difluoromethyl)-1H-pyrazole

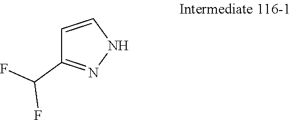

### Synthetic Route:

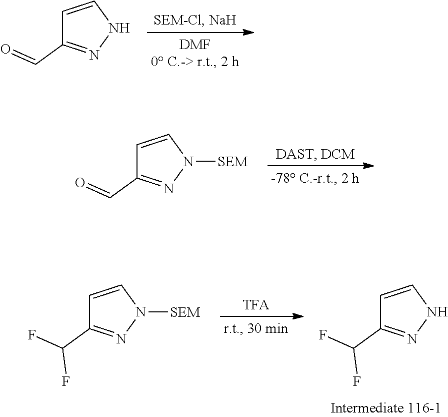 [SMILES: O=Cc1cc[nH]n1 ; C[Si](C)(C)CCOCn1ccc(C=O)n1 ; C[Si](C)(C)CCOCn1ccc(C(F)F)n1 ; FC(F)c1cc[nH]n1]

### Step 1: Synthesis of 1-((2-(trimethylsilyl)ethoxy)methyl)-1H-pyrazole-3-carbaldehyde

To a cooled (0° C.) solution of 1H-pyrazole-3-carbaldehyde (4.00 g, 41.6 mmol, 1 equiv) in N,N-dimethylformamide (50 mL) was added sodium hydride (60% dispersion, 3.33 g, 83.3 mmol, 2 equiv) and the resulting solution was stirred at 0° C. for 30 min. (2-(chloromethoxy)ethyl)trimethylsilane (8.33 g, 50.0 mmol, 1.2 equiv) was added and the mixture was stirred at room temperature for 2 h. The reaction mixture was quenched with water (200 mL) and the resulting mixture was extracted with ethyl acetate (3×200 mL). The combined organic layers were washed with brine (50 mL), dried over anhydrous sodium sulfate, filtered, and concentrated in vacuo to afford crude 1-((2-(trimethylsilyl)ethoxy)methyl)-1H-pyrazole-3-carbaldehyde (5.2 g, 47%) as a colorless oil, which was used directly in the next step without purification. MS (ESI) calculated for C₁₀H₁₈N₂O₂Si: 226.11 m/z, found 227.15 [M+H]⁺.

### Step 2: Synthesis of 3-(difluoromethyl)-1-((2-(trimethylsilyl)ethoxy)methyl)-1H-pyrazole

To a cooled (−78° C.) solution of 1-((2-(trimethylsilyl)ethoxy)methyl)-1H-pyrazole-3-carbaldehyde (4.20 g, 18.6 mmol, 1 equiv) in dichloromethane (50 mL) was added diethylaminosulfur trifluoride (8.97 g, 55.7 mmol, 3 equiv) and the resulting mixture was stirred at −78° C. for 2 h. The reaction was quenched by the addition of saturated aqueous ammonium chloride (100 mL) and the resulting mixture was extracted with ethyl acetate (3×200 mL). The combined organic layers were washed with brine (50 mL), dried over anhydrous sodium sulfate, filtered, and concentrated in vacuo. The resulting residue was purified by reverse-phase flash column chromatography on C18 silica gel using a 0-90% gradient of acetonitrile in water (+0.05% ammonium bicarbonate) to afford 3-(difluoromethyl)-1-((2-(trimethylsilyl)ethoxy)methyl)-1H-pyrazole (560 mg, 9%) as a yellow oil. MS (ESI) calculated for C₁₀H₁₈F₂N₂OSi: 248.12 m/z, found 249.05 [M+H]⁺.

### Step 3: Synthesis of 3-(difluoromethyl)-1H-pyrazole (Intermediate 116-1)

3-(difluoromethyl)-1-((2-(trimethylsilyl)ethoxy)methyl)-1H-pyrazole (560 mg, 2.256 mmol, 1 equiv) was dissolved in 2,2,2-trifluoroacetic acid (5 mL) and the resulting mixture was stirred at room temperature for 15 min. The resulting mixture was concentrated in vacuo and purified by reverse-phase flash column chromatography on C18 silica gel using a 0-50% gradient of acetonitrile in water (+0.05% ammonium bicarbonate) to afford 3-(difluoromethyl)-1H-pyrazole (Intermediate 116-1) (170 mg, 46%) as a yellow oil. MS (ESI) calculated for C₄H₄F₂N₂: 118.03 m/z, found 117.05 [M−H]⁻.

### Example 117: (S)—N-(5-(5-(1′H-[1,3′-bipyrazol]-1′-yl)-2-(2-aminopyridin-3-yl)-3H-imidazo[4,5-b]pyridin-3-yl)-2,3-dihydro-1H-inden-1-yl)-3-formyl-4-hydroxybenzamide

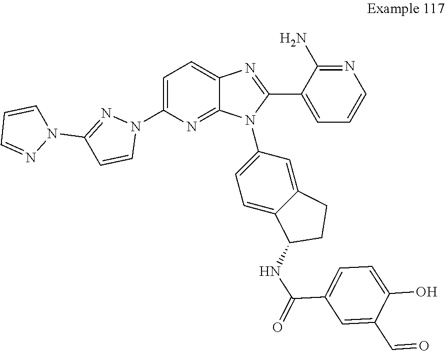 [SMILES: Nc1ncccc1-c1nc2ccc(-n3ccc(-n4cccn4)n3)nc2n1-c1ccc2c(c1)CC[C@@H]2NC(=O)c1ccc(O)c(C=O)c1]

Example 117 was prepared in a manner analogous to Example 94 using Intermediate 117-2 in place of 4-(difluoromethyl)-1H-pyrazole. MS (ESI) calculated for C₃₄H₂₆N₁₀O₃: 622.22 m/z, found 645.25 [M+Na]⁺. ¹H NMR (400 MHz, DMSO-d₆) δ (ppm): 10.25 (s, 1H), 8.40-8.43 (m, 3H), 8.02-8.30 (m, 1H), 7.90-8.01 (m, 3H), 7.71-7.79 (m, 1H), 7.21-7.45 (m, 4H), 6.72-7.16 (m, 2H), 6.51-6.69 (m, 1H), 6.40-6.65 (m, 1H), 5.52-5.70 (m, 1H), 2.98-3.10 (m, 1H), 2.77-2.97 (m, 1H), 2.29-2.41 (m, 1H), 2.01-2.18 (m, 1H).

### Intermediate 117-2: 1′H-1,3′-bipyrazole

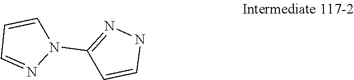

### Synthetic Route:

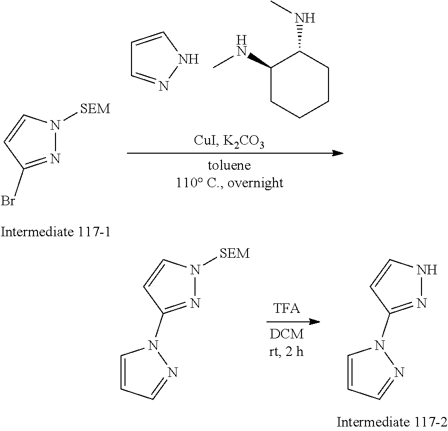

### Step 1: Synthesis of 1′-{[2-(trimethylsilyl)ethoxy]methyl}-1,3′-bipyrazole

A mixture of 3-bromo-1-{[2-(trimethylsilyl)ethoxy]methyl}pyrazole (Intermediate 117-1) (8.00 g, 28.9 mmol, 1 equiv), pyrazole (1.96 g, 28.9 mmol, 1 equiv), (1R,2R)—N1,N2-dimethylcyclohexane-1,2-diamine (1.64 g, 11.5 mmol, 0.4 equiv), copper (I) iodide (0.55 g, 2.9 mmol, 0.1 equiv) and potassium carbonate (8.77 g, 63.5 mmol, 2.2 equiv) in toluene (150 mL) was stirred at 110° C. overnight under nitrogen atmosphere. The resulting mixture was cooled to room temperature and quenched with water (150 mL). The mixture was extracted with ethyl acetate (3×150 mL). The organic layers were combined, dried over anhydrous sodium sulfate, filtered, and concentrated in vacuo. The residue obtained was purified by silica gel column chromatography using a 0-20% gradient of ethyl acetate in petroleum ether to afford 1′-{[2-(trimethylsilyl)ethoxy]methyl}-1,3′-bipyrazole (2 g, 23%) as a yellow oil. MS (ESI) calculated for C₁₂H₂₀N₄OSi: 264.14 m/z, found 265.10 [M+H]⁺.

### Step 2: Synthesis of 1′H-1,3′-bipyrazole (Intermediate 117-2)

To a solution of 1′-{[2-(trimethylsilyl)ethoxy]methyl}-1,3′-bipyrazole (1.8 g, 6.8 mmol, 1 equiv) in DCM (25 mL) was added 2,2,2-trifluoroacetic acid (25 mL) and the resulting mixture was stirred for 1 h at room temperature. The reaction mixture was concentrated in vacuo and taken up into ammonium hydroxide (10 mL) and methanol (20 mL). The mixture was stirred for 1 h at room temperature then concentrated in vacuo to afford 1′H-1,3′-bipyrazole (Intermediate 117-2) (800 mg, 88%), which was used without purification in subsequent transformations. MS (ESI) calculated for C₆H₆N₄: 134.06 m/z, found 135.16 [M+H]⁺.

### Intermediate 117-1: 3-bromo-1-((2-(trimethylsilyl)ethoxy)methyl)-1H-pyrazole

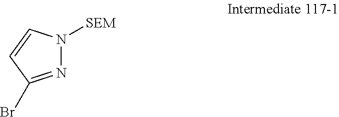 [SMILES: C[Si](C)(C)CCOCn1ccc(Br)n1]

### Step 1: Synthesis of 3-bromo-1-((2-(trimethylsilyl)ethoxy)methyl)-1H-pyrazole (Intermediate 117-1)

To a cooled (0° C.) solution of 3-bromo-1H-pyrazole (10 g, 68 mmol, 1 equiv) in tetrahydrofuran (700 mL) was added sodium hydride (60% dispersion, 4.08 g, 102 mmol, 1.5 equiv). The mixture was stirred for 30 min at 0° C. followed by addition of (2-(chloromethoxy)ethyl)trimethylsilane (17.02 g, 102 mmol, 1.5 equiv). The mixture was allowed to slowly warm to room temperature and stirring was continued for 1 h. The reaction mixture was quenched with water and extracted with ethyl acetate (3×500 mL). The combined organic layers were washed with brine, dried over anhydrous sodium sulfate, filtered, and concentrated in vacuo. The residue was purified by silica gel column chromatography, eluting with 17% ethyl acetate in petroleum ether to afford 3-bromo-1-{[2-(trimethylsilyl)ethoxy]methyl}pyrazole (13.6 g, 61%) as a white oil. MS (ESI) calculated for C₉H₁₇BrN₂OSi: 276.03 m/z, found 276.95 [M+H]⁺, 278.95 [M+H+2]⁺.

### Example 118: (S)—N-(5-(2-(2-aminopyridin-3-yl)-5-(2H-1,2,3-triazol-2-yl)-3H-imidazo[4,5-b]pyridin-3-yl)-2,3-dihydro-1H-inden-1-yl)-2,3-difluoro-5-formyl-4-hydroxybenzamide

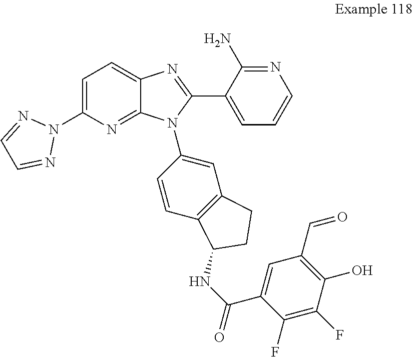 [SMILES: Nc1ncccc1-c1nc2ccc(-n3nccn3)nc2n1-c1ccc2c(c1)CC[C@@H]2NC(=O)c1cc(C=O)c(O)c(F)c1F]

Example 118 was prepared in a manner analogous to Example 12 using Intermediate 91-2 in place of Intermediate 1-3 and Intermediate 118-1 in place of Intermediate 12-1. MS (ESI) calculated for C₃₀H₂₁F₂N₉O₃: 593.17 m/z, found 594.20 [M+H]⁺. ¹H NMR (400 MHz, DMSO-d₆) δ (ppm): 10.20 (s, 1H), 8.48-8.50 (m, 1H), 8.04-8.13 (m, 4H), 7.79-7.84 (m, 2H), 7.37-7.45 (m, 3H), 6.79-6.82 (m, 1H), 5.53-5.57 (m, 1H), 2.98-3.04 (m, 1H), 2.85-2.91 (m, 1H), 2.50-2.58 (m, 1H), 2.02-2.08 (m, 1H). ¹⁹F NMR (376 MHz, DMSO-d₆) δ (ppm): −158.16, −128.26.

### Intermediate 118-1: 4-(benzyloxy)-5-(1,3-dioxolan-2-yl)-2,3-difluorobenzoic acid

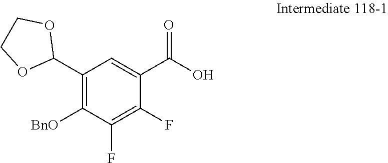 [SMILES: O=C(O)c1cc(C2OCCO2)c(OCc2ccccc2)c(F)c1F]

Intermediate 118-1 was prepared in a manner analogous to Intermediate 110-1 using 2,3-difluoro-4-hydroxybenzoic acid in place of 4-bromo-3-chloro-2-fluorophenol. MS (ESI) calculated for C₁₇H₁₄F₂O₅: 336.08 m/z, found 337.10 [M+H]⁺.

### Example 119: N-[(1S)-5-[2-(2-aminopyridin-3-yl)-5-(3-ethynylpyrazol-1-yl)imidazo[4,5-b]pyridin-3-yl]-2,3-dihydro-1H-inden-1-yl]-3-formyl-4-hydroxybenzamide

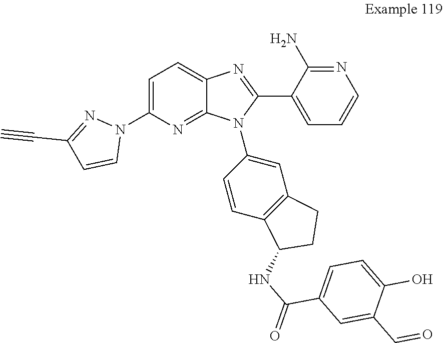 [SMILES: C#Cc1ccn(-c2ccc3nc(-c4cccnc4N)n(-c4ccc5c(c4)CC[C@@H]5NC(=O)c4ccc(O)c(C=O)c4)c3n2)n1]

Example 119 was prepared in a manner analogous to Example 94 using Intermediate 119-2 in place of 4-(difluoromethyl)-1H-pyrazole. MS (ESI) calculated for C₃₃H₂₄N₈O₃: 580.20 m/z, found 581.25 [M+H]⁺. ¹H NMR (400 MHz, DMSO-d₆) δ (ppm): 10.25 (s, 1H), 8.82-8.91 (m, 1H), 8.15-8.25 (m, 2H), 7.95-8.06 (m, 2H), 7.72-7.85 (m, 1H), 7.56-7.65 (m, 1H), 7.25-7.39 (m, 3H), 7.20-7.25 (m, 1H), 6.88-7.01 (m, 1H), 6.52-6.65 (m, 1H), 6.40-6.49 (m, 1H), 5.55-5.65 (m, 1H), 3.60-3.82 (m, 1H), 2.95-3.09 (m, 1H), 2.81-2.86 (m, 1H), 2.42-2.58 (m, 1H), 1.98-2.11 (m, 1H).

### Intermediate 119-2: 3-((trimethylsilyl)ethynyl)-1H-pyrazole

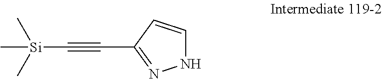

### Synthetic Route:

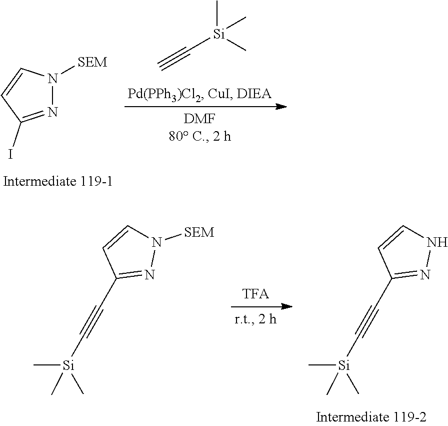

### Step 1: Synthesis of 1-{[2-(trimethylsilyl)ethoxy]methyl}-3-[2-(trimethylsilyl)ethynyl]pyrazole

To a solution of 3-iodo-1-{[2-(trimethylsilyl)ethoxy]methyl}pyrazole (Intermediate 119-1) (2.00 g, 7.21 mmol, 1 equiv) in N,N-dimethylformamide (12 mL) was added copper (I) iodide (0.070 g, 0.36 mmol, 0.05 equiv), N,N-diisopropylethylamine (1.86 g, 14.4 mmol, 2 equiv), bis(triphenylphosphine)palladium(II) dichloride (0.25 g, 0.36 mmol, 0.05 equiv) and trimethylsilylacetylene (4.25 g, 43.3 mmol, 6 equiv). The resulting mixture was stirred at 80° C. for 2 h under a nitrogen atmosphere. The reaction mixture was cooled to room temperature and concentrated in vacuo. The residue was purified by silica gel column chromatography using 0-95% gradient of ethyl acetate in petroleum ether to afford 1-{[2-(trimethylsilyl)ethoxy]methyl}-3-[2-(trimethylsilyl)ethynyl]pyrazole (1.4 g, 66%) as a yellow oil. MS (ESI) calculated for C₁₄H₂₆N₂OSi₂: 294.16 m/z, found 295.20 [M+H]⁺.

### Step 2: Synthesis of 3-[2-(trimethylsilyl)ethynyl]-1H-pyrazole (Intermediate 119-2)

A solution of 1-{[2-(trimethylsilyl)ethoxy]methyl}-3-[2-(trimethylsilyl)ethynyl]pyrazole (1.4 g, 4.8 mmol, 1 equiv) in 2,2,2-trifluoroacetic acid (20 mL) was stirred at room temperature for 2 h. The mixture was concentrated in vacuo and the residue was purified by reverse-phase flash column chromatography on C18 silica gel using a 0-95% gradient of acetonitrile in water (+0.05% ammonium bicarbonate) to afford 3-[2-(trimethylsilyl) ethynyl]-1H-pyrazole (Intermediate 119-2) (576 mg, 74%) as a yellow oil. MS (ESI) calculated for C₈H₁₂N₂Si: 164.08 m/z, found 165.10 [M+H]⁺.

### Intermediate 119-1: 3-iodo-1-((2-(trimethylsilyl)ethoxy)methyl)-1H-pyrazole

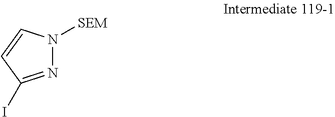 [SMILES: C[Si](C)(C)CCOCn1ccc(I)n1]

Intermediate 119-1 was prepared in a manner analogous to Intermediate 117-1 using 3-iodo-1H-pyrazole in place of 3-bromo-1H-pyrazole. MS (ESI) calculated for C₉H₁₇IN₂OSi: 324.02 m/z, found 325.00 [M+H]⁺.

### Example 120: N-[(1S)-5-[2-(2-aminopyridin-3-yl)-5-(2-methyl-1,2,3-triazol-4-yl)imidazo[4,5-b]pyridin-3-yl]-2,3-dihydro-1H-inden-1-yl]-3-formyl-4-hydroxybenzamide

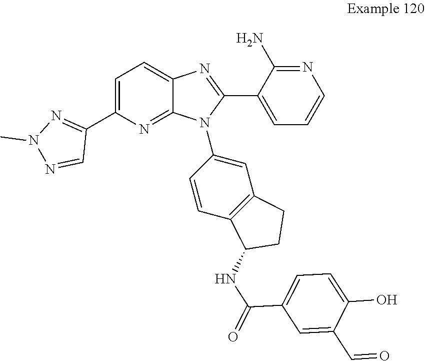 [SMILES: Cn1ncc(-c2ccc3nc(-c4cccnc4N)n(-c4ccc5c(c4)CC[C@@H]5NC(=O)c4ccc(O)c(C=O)c4)c3n2)n1]

Example 120 was prepared in a manner analogous to Example 113 using Intermediate 120-1 in place of the boronate ester and bis(di-tert-butylphosphino)ferrocene]dichloropalladium(II)/tribasic potassium phosphate/2 h instead of tetrakis(triphenylphosphine)palladium(0)/sodium carbonate/1 h. MS (ESI) calculated for C₃₁H₂₅N₉O₃: 571.21 m/z, found 572.30 [M+H]⁺. ¹H NMR (400 MHz, DMSO-d₆) δ (ppm): 10.23 (s, 1H), 8.35 (s, 1H), 8.18-8.30 (m, 2H), 7.89-8.06 (m, 4H), 7.20-7.35 (m, 4H), 6.91-7.02 (m, 1H), 6.42-6.50 (m, 1H), 5.55-5.65 (m, 1H), 4.15-4.25 (m, 3H), 2.95-3.09 (m, 1H), 2.80-2.95 (m, 1H), 2.42-2.60 (m, 1H), 2.01-2.15 (m, 1H).

### Intermediate 120-1: (2-methyl-2H-1,2,3-triazol-4-yl)boronic acid

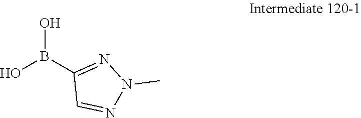

Intermediate 120-1 was prepared in a manner analogous to Intermediate 60-1 using 4-bromo-2-methyl-1,2,3-triazole in place of 2-bromo-6-methylpyrazine. MS (ESI) calculated for C₃H₆BN₃O₂: 127.06 m/z, found 128.10 [M+H]⁺.

### Example 121: (S)—N-(5-(2-(2-aminopyridin-3-yl)-5-(oxazol-2-yl)-3H-imidazo[4,5-b]pyridin-3-yl)-2,3-dihydro-1H-inden-1-yl)-3-formyl-4-hydroxybenzamide

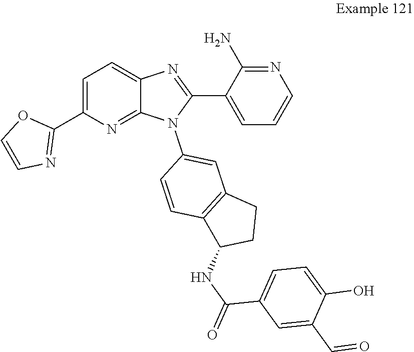 [SMILES: Nc1ncccc1-c1nc2ccc(-c3ncco3)nc2n1-c1ccc2c(c1)CC[C@@H]2NC(=O)c1ccc(O)c(C=O)c1]

### Synthetic Route:

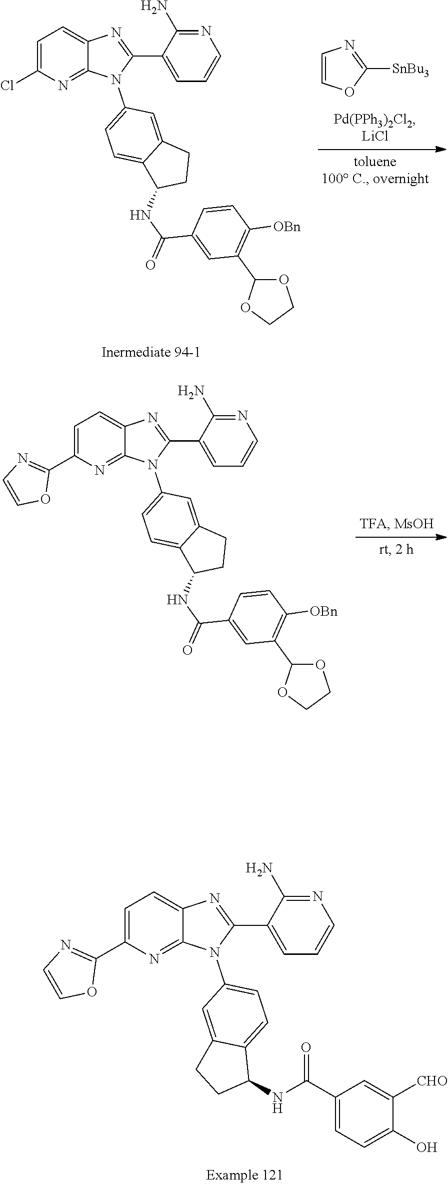

### Step 1: Synthesis of (S)—N-(5-(2-(2-aminopyridin-3-yl)-5-(oxazol-2-yl)-3H-imidazo[4,5-b]pyridin-3-yl)-2,3-dihydro-1H-inden-1-yl)-4-(benzyloxy)-3-(1,3-dioxolan-2-yl)benzamide

A suspension of N-[(1S)-5-[2-(2-aminopyridin-3-yl)-5-chloroimidazo[4,5-b]pyridin-3-yl]-2,3-dihydro-1H-inden-1-yl]-4-(benzyloxy)-3-(1,3-dioxolan-2-yl)benzamide (Intermediate 94-1) (200 mg, 0.303 mmol, 1 equiv), 2-(tributylstannyl)-1,3-oxazole (543 mg, 1.52 mmol, 5 equiv), bis(triphenylphosphine)palladium(II) dichloride (21 mg, 0.030 mmol, 0.1 equiv) and lithium chloride (51 mg, 1.2 mmol, 4 equiv) in toluene (5 mL) was stirred at 100° C. overnight under nitrogen atmosphere. The resulting mixture was cooled to room temperature and quenched by addition of water (20 mL). The mixture was extracted with ethyl acetate (20 mL×3). The combined organic phases were washed with brine (30 mL×3), dried with sodium sulfate, filtered, and concentrated in vacuo. The residue was purified by reverse-phase column chromatography using a 0-95% gradient of acetonitrile in water (+0.1% formic acid) to afford (S)—N-(5-(2-(2-aminopyridin-3-yl)-5-(oxazol-2-yl)-3H-imidazo[4,5-b]pyridin-3-yl)-2,3-dihydro-1H-inden-1-yl)-4-(benzyloxy)-3-(1,3-dioxolan-2-yl)benzamide (150 mg, 68%) as a yellow solid. MS (ESI) calculated for C₄₀H₃₃N₇O₅: 691.25 m/z, found 692.20 [M+H]⁺.

### Step 2: Synthesis of (S)—N-(5-(2-(2-aminopyridin-3-yl)-5-(oxazol-2-yl)-3H-imidazo[4,5-b]pyridin-3-yl)-2,3-dihydro-1H-inden-1-yl)-3-formyl-4-hydroxybenzamide (Example 121)

N-[(1S)-5-[2-(2-aminopyridin-3-yl)-5-(1,3-oxazol-2-yl)imidazo[4,5-b]pyridin-3-yl]-2,3-dihydro-1H-inden-1-yl]-4-(benzyloxy)-3-(1,3-dioxolan-2-yl)benzamide (198 mg, 0.286 mmol, 1 equiv) was dissolved in 2,2,2-trifluoroacetic acid (5 mL) and methanesulfonic acid (1 mL) was added. The resulting mixture was stirred at room temperature for 30 min then concentrated in vacuo and purified by preparative HPLC on a XSelect CSH column using a 20-40% gradient of acetonitrile in water (+0.05% 2,2,2-trifluoroacetic acid) to afford (S)—N-(5-(2-(2-aminopyridin-3-yl)-5-(oxazol-2-yl)-3H-imidazo[4,5-b]pyridin-3-yl)-2,3-dihydro-1H-inden-1-yl)-3-formyl-4-hydroxybenzamide (Example 121) (15.6 mg, 10%) as a yellow solid. MS (ESI) calculated for C₃₁H₂₃N₇O₄: 557.18 m/z, found 558.20 [M+H]⁺. ¹H NMR (400 MHz, DMSO-d₆) δ (ppm): 10.14 (s, 1H), 8.33 (s, 1H), 8.11-8.22 (m, 2H), 8.06 (s, 1H), 8.00 (s, 1H), 7.93 (s, 1H), 7.70-7.76 (m, 1H), 7.30-7.40 (m, 3H), 7.23-7.28 (m, 1H), 7.00-7.06 (m, 1H), 6.74-6.82 (m, 1H), 5.49-5.59 (m, 1H), 2.96-3.06 (m, 1H), 2.80-2.92 (m, 1H), 2.52-2.60 (m, 1H), 1.98-2.10 (m, 1H).

### Example 122: N-[(1S)-5-[2-(2-aminopyridin-3-yl)-5-(3-fluoropyrazol-1-yl)imidazo[4,5-b]pyridin-3-yl]-2,3-dihydro-1H-inden-1-yl]-4-[(dimethylcarbamoyl)amino]-3-formylbenzamide

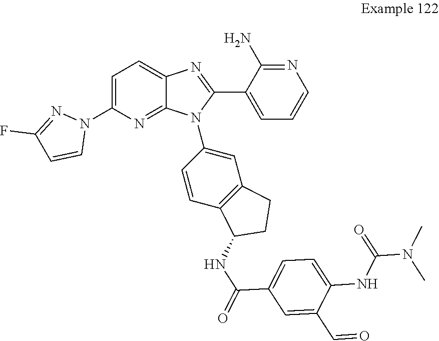 [SMILES: CN(C)C(=O)Nc1ccc(C(=O)N[C@H]2CCc3cc(-n4c(-c5cccnc5N)nc5ccc(-n6ccc(F)n6)nc54)ccc32)cc1C=O]

Example 122 was prepared in a manner analogous to Example 12 using Intermediate 75-1 in place of Intermediate 1-3, Intermediate 63-2 in place of Intermediate 12-1 and 2,2,2-trifluoroacetic acid/dichloromethane (5:1) in place of 2,2,2-trifluoroacetic acid/methanesulfonic acid. MS (ESI) calculated for C₃₄H₂₉FN₁₀O₃: 644.24 m/z, found 645.30 [M+H]⁺. ¹H NMR (400 MHz, DMSO-d₆) δ (ppm): 9.98 (s, 1H), 8.48-8.51 (m, 1H), 8.44-8.45 (m, 1H), 8.33-8.35 (m, 1H), 8.27-8.29 (m, 1H), 8.14-8.17 (m, 1H), 7.99-8.01 (m, 1H), 7.75-7.78 (m, 1H), 7.36-7.38 (m, 2H), 7.26-7.29 (m, 2H), 6.45-6.48 (m, 1H), 6.32-6.34 (m, 1H), 5.61-5.65 (m, 1H), 3.04-3.07 (m, 7H), 2.88-2.95 (m, 1H), 2.54 (s, 1H), 2.09-2.12 (m, 1H). ¹⁹F-NMR (400 MHz, DMSO-d₆) δ (ppm): −127.18.

### Example 123: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-chloro-2-fluoro-5-formyl-4-hydroxybenzamide

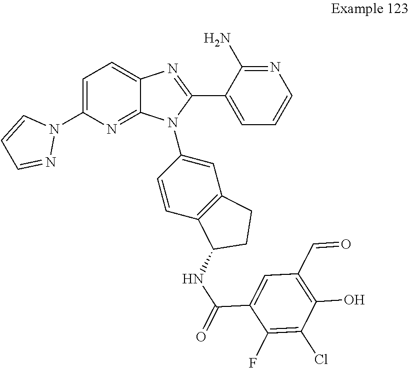 [SMILES: Nc1ncccc1-c1nc2ccc(-n3cccn3)nc2n1-c1ccc2c(c1)CC[C@@H]2NC(=O)c1cc(C=O)c(O)c(Cl)c1F]

Example 123 was prepared in a manner analogous to Example 12 using Intermediate 33-1 in place of Intermediate 1-3 and Intermediate 123-2 in place of Intermediate 12-1. MS (ESI) calculated for C₃₁H₂₂ClFN₈O₃: 608.15 m/z, found 609.15 [M+H]⁺. ¹H NMR (400 MHz, DMSO-d₆) δ (ppm): 10.13 (s, 1H), 8.35-8.37 (m, 2H), 8.01-8.08 (m, 3H), 7.80-7.85 (m, 2H), 7.36-7.47 (m, 3H), 6.81-6.87 (m, 1H), 6.56-6.57 (m, 1H), 5.54-5.58 (m, 1H), 3.00-3.06 (m, 1H), 2.87-2.95 (m, 1H), 2.55-2.58 (m, 1H), 2.00-2.06 (m, 1H). ¹⁹F NMR (376 MHz, DMSO-d₆) δ (ppm): −102.98.

### Intermediate 123-2: 4-(benzyloxy)-3-chloro-5-(1,3-dioxolan-2-yl)-2-fluorobenzoic acid

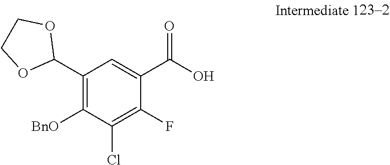 [SMILES: O=C(O)c1cc(C2OCCO2)c(OCc2ccccc2)c(Cl)c1F]

Intermediate 123-2 was prepared in a manner analogous to Intermediate 110-1 using Intermediate 123-1 in place of 4-bromo-3-chloro-2-fluorophenol. MS (ESI) calculated for C₁₇H₁₄ClFO₅: 352.05 m/z, found 351.00 [M+H]⁻.

### Intermediate 123-1: 4-bromo-2-chloro-3-fluorophenol

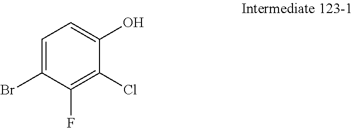

### Synthetic Route:

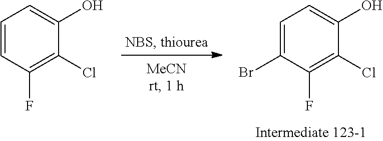 [SMILES: Oc1cccc(F)c1Cl ; Oc1ccc(Br)c(F)c1Cl]

### Step 1: Synthesis of 4-bromo-2-chloro-3-fluorophenol (Intermediate 123-1)

To a solution of 2-chloro-3-fluorophenol (8.00 g, 54.6 mmol) in acetonitrile (600 mL) were added thiourea (0.40 g, 5.5 mmol, 0.1 equiv) and N-bromosuccinimide (7.76 g, 43.6 mmol, 0.8 equiv). The resulting solution was stirred at room temperature for 2 h. The mixture was then concentrated in vacuo and purified by silica gel column chromatography using a 0-10% gradient of ethyl acetate in petroleum ether to afford 4-bromo-2-chloro-3-fluorophenol (Intermediate 123-1) as a white solid (3.1 g, 50%). MS (ESI) calculated for C₆H₃BrClFO: 223.90 m/z, found 224.80 [M+H]⁺.

### Example 124: N-[(1S)-5-[2-(2-aminopyridin-3-yl)-5-[3-(methylsulfanyl) pyrazol-1-yl]imidazo[4,5-b]pyridin-3-yl]-2,3-dihydro-1H-inden-1-yl]-3-formyl-4-hydroxybenzamide

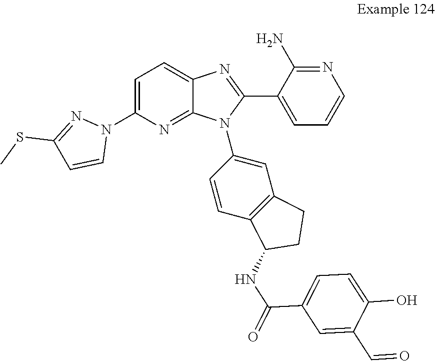 [SMILES: CSc1ccn(-c2ccc3nc(-c4cccnc4N)n(-c4ccc5c(c4)CC[C@@H]5NC(=O)c4ccc(O)c(C=O)c4)c3n2)n1]

Example 124 was prepared in a manner analogous to Example 94 using Intermediate 124-1 in place of 4-(difluoromethyl)-1H-pyrazole. MS (ESI) calculated for C₃₂H₂₆N₈O₃S: 602.18 m/z, found 603.20 [M+H]⁺. ¹H NMR (300 MHz, DMSO-d₆) δ (ppm): 10.32 (s, 1H), 8.28-8.59 (m, 3H), 7.90-8.28 (m, 3H), 7.71-7.81 (m, 1H), 7.28-7.49 (m, 3H), 7.05-7.19 (m, 1H), 6.75-6.90 (m, 1H), 6.55-6.62 (m, 1H), 5.56-5.70 (m, 1H), 2.82-3.13 (m, 2H), 2.55-2.62 (m, 3H), 2.02-2.52 (m, 2H).

### Intermediate 124-1: 3-(methylthio)-1H-pyrazole

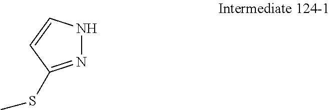

### Synthetic Route:

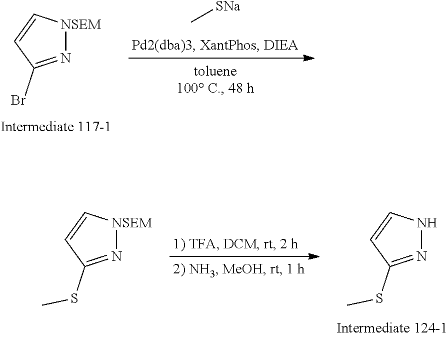

### Step 1: Synthesis of 3-(methylsulfanyl)-1-{[2-(trimethylsilyl)ethoxy]methyl}pyrazole

A mixture of 3-bromo-1-{[2-(trimethylsilyl)ethoxy]methyl}pyrazole (Intermediate 117-1) (3.80 g, 13.7 mmol, 1 equiv), (methylsulfanyl)sodium (4.80 g, 68.5 mmol, 5 equiv) and N,N-diisopropylethylamine (5.31 g, 41.1 mmol, 3 equiv) were dissolved in toluene (4 mL). Then XantPhos (0.79 g, 1.4 mmol, 0.1 equiv) and tris(dibenzylideneacetone)dipalladium(0) (1.26 g, 1.37 mmol, 0.1 equiv) were added and stirred overnight at 100° C. under N₂ atmosphere. The mixture was cooled to room temperature and quenched by the addition of water (200 mL). The mixture was extracted with ethyl acetate (3×200 mL). The combined organic layers were washed with brine (3×200 mL), dried over anhydrous sodium sulfate, filtered, and concentrated under reduced pressure. The residue was purified by silica gel column chromatography using a 0-15% gradient of ethyl acetate in petroleum ether to afford 3-(methylsulfanyl)-1-{[2-(trimethylsilyl)ethoxy]methyl}pyrazole (910 mg, 27%) as a yellow oil. MS (ESI) calculated for C₁₀H₂₀N₂OSSi: 244.11 m/z, found 245.15 [M+H]⁺.

### Step 2: Synthesis of 3-(methylsulfanyl)-1H-pyrazole (Intermediate 124-1)

To a solution of 3-(methylsulfanyl)-1-{[2-(trimethylsilyl)ethoxy]methyl}pyrazole (850 mg, 3.477 mmol, 1 equiv) in dichloromethane (5 mL) was added 2,2,2-trifluoroacetic acid (5 mL) and the resulting mixture was stirred for 2 h at room temperature. The mixture was concentrated under reduced pressure and the residue was dissolved in 30% aqueous ammonia (6 mL) and methanol (6 mL) The resulting mixture was stirred for 1 h at room temperature. The mixture was concentrated under reduced pressure and the crude residue was purified by silica gel column chromatography. Using a 0-20% gradient of ethyl acetate in petroleum ether to afford 3-(methylsulfanyl)-1H-pyrazole (210 mg, 53%) as a yellow green solid. MS (ESI) calculated for C₄H₆N₂S: 114.03 m/z, found 115.10 [M+H]⁺.

### Example 125: (S)—N-(5-(2-(2-aminopyridin-3-yl)-5-(3-fluoro-1H-pyrazol-1-yl)-3H-imidazo[4,5-b]pyridin-3-yl)-2,3-dihydro-1H-inden-1-yl)-2,3-difluoro-5-formyl-4-hydroxybenzamide

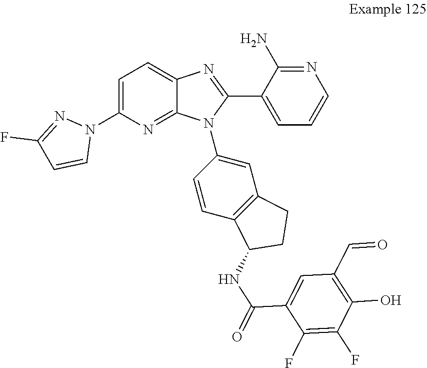 [SMILES: Nc1ncccc1-c1nc2ccc(-n3ccc(F)n3)nc2n1-c1ccc2c(c1)CC[C@@H]2NC(=O)c1cc(C=O)c(O)c(F)c1F]

Example 125 was prepared in a manner analogous to Example 12 using Intermediate 75-1 in place of Intermediate 1-3 and Intermediate 118-1 in place of Intermediate 12-1. MS (ESI) calculated for C₃₁H₂₁F₃N₈O₃: 610.17 m/z, found 611.20 [M+H]⁺. ¹H NMR (300 MHz, DMSO-d₆) δ (ppm): 10.23 (s, 1H), 8.35-8.37 (m, 1H), 8.30-8.32 (m, 1H), 8.01-8.04 (m, 1H), 7.84-7.88 (m, 1H), 7.76-7.78 (m, 1H), 7.46-7.37 (m, 2H), 7.25-7.31 (m, 2H), 6.42-6.48 (m, 1H), 6.36-6.39 (m, 1H), 5.54-5.59 (m, 1H), 2.88-3.05 (m, 2H), 2.08-2.11 (m, 1H), 1.94-2.01 (m, 1H).

### Example 126: N-[(1S)-5-[2-(2-aminopyridin-3-yl)-5-[3-(methoxymethyl)pyrazol-1-yl]imidazo[4,5-b]pyridin-3-yl]-2,3-dihydro-1H-inden-1-yl]-3-formyl-4-hydroxybenzamide

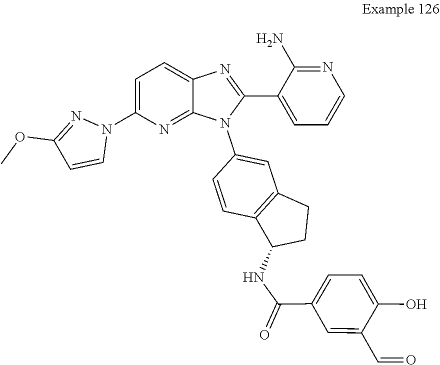 [SMILES: COc1ccn(-c2ccc3nc(-c4cccnc4N)n(-c4ccc5c(c4)CC[C@@H]5NC(=O)c4ccc(O)c(C=O)c4)c3n2)n1]

Example 126 was prepared in a manner analogous to Example 94 using 3-(methoxymethyl)-1H-pyrazole in place of 4-(difluoromethyl)-1H-pyrazole. MS (ESI) calculated for C₃₃H₂₈N₈O₄: 600.22 m/z, found 601.25 [M+H]⁺. ¹H NMR (300 MHz, DMSO-d₆) δ (ppm): 10.32 (s, 1H), 8.34-8.35 (m, 1H), 8.31-8.32 (m, 2H), 8.09-8.10 (m, 2H), 7.96-7.98 (m, 1H), 7.71-7.72 (m, 1H), 7.45-7.46 (m, 2H), 7.35-7.36 (m, 2H), 7.08-7.10 (m, 1H), 6.77-6.78 (m, 1H), 5.60-5.65 (m, 1H), 4.41-4.47 (m, 2H), 3.38-3.41 (m, 3H), 3.18-3.19 (m, 1H), 2.90-2.91 (m, 1H), 2.73-2.74 (m, 1H), 2.11-2.31 (m, 1H).

### Example 127: (S)—N-(5-(2-(2-aminopyridin-3-yl)-5-(3-(hydroxymethyl)-1H-pyrazol-1-yl)-3H-imidazo[4,5-b]pyridin-3-yl)-2,3-dihydro-1H-inden-1-yl)-3-formyl-4-hydroxybenzamide

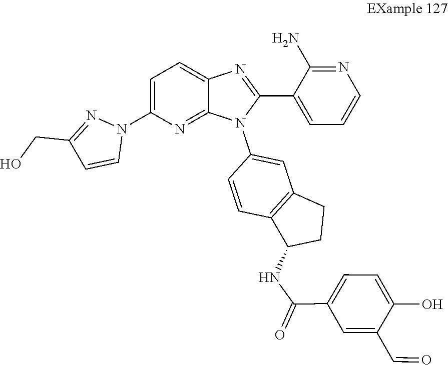 [SMILES: Nc1ncccc1-c1nc2ccc(-n3ccc(CO)n3)nc2n1-c1ccc2c(c1)CC[C@@H]2NC(=O)c1ccc(O)c(C=O)c1]

Example 127 was prepared in manner analogous to Example 94 using (1H-pyrazol-3-yl)methanol in place of 4-(difluoromethyl)-1H-pyrazole. Note that both O- and N-addition products were observed in Step 1. The products were separated and carried through to Step 2 to produce Example 127 and Example 128 (vide infra). MS (ESI) calculated for C₃₂H₂₆N₈O₄: 586.21 m/z, found 587.25 [M+H]⁺. ¹H NMR (400 MHz, DMSO-d₆) δ (ppm): 10.31 (s, 1H), 8.38-8.40 (m, 1H), 8.30-8.31 (m, 2H), 8.05-8.12 (m, 2H), 7.93-7.95 (m, 1H), 7.65-7.69 (m, 1H), 7.43-7.44 (m, 1H), 7.33-7.35 (m, 2H), 7.07-7.10 (m, 1H), 6.74-7.76 (m, 1H), 6.52-6.53 (m, 1H), 5.61-5.63 (m, 1H), 4.52 (s, 2H), 3.00-3.06 (m, 1H), 2.87-2.95 (m, 1H), 2.50-2.51 (m, 1H), 2.05-2.08 (m, 1H).

### Example 128: (S)—N-(5-(5-((1H-pyrazol-3-yl)methoxy)-2-(2-aminopyridin-3-yl)-3H-imidazo[4,5-b]pyridin-3-yl)-2,3-dihydro-1H-inden-1-yl)-3-formyl-4-hydroxybenzamide

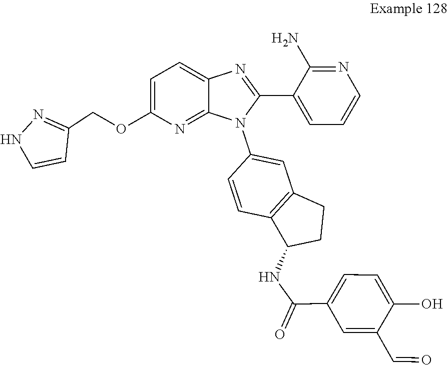 [SMILES: Nc1ncccc1-c1nc2ccc(OCc3cc[nH]n3)nc2n1-c1ccc2c(c1)CC[C@@H]2NC(=O)c1ccc(O)c(C=O)c1]

Example 128 was prepared in manner analogous to Example 94 using (1H-pyrazol-3-yl)methanol in place of 4-(difluoromethyl)-1H-pyrazole. Note that both O- and N-addition products were observed in Step 1. The products were separated and carried through to Step 2 to produce Example 127 (vide supra) and Example 128. MS (ESI) calculated for C₃₂H₂₆N₈O₄: 586.21 m/z, found 587.15 [M+H]⁺. ¹H NMR (400 MHz, DMSO-d₆) δ (ppm): 10.10 (s, 1H), 8.36-8.47 (m, 1H), 8.01-8.02 (m, 2H), 7.85-7.88 (m, 2H), 7.67-7.68 (m, 1H), 7.32-7.35 (m, 1H), 7.30-7.31 (m, 2H), 7.29-7.30 (m, 1H), 6.46-6.48 (m, 3H), 5.56-5.59 (m, 1H), 4.76-4.78 (m, 2H), 2.90-3.00 (m, 2H), 2.50-2.51 (m, 1H), 2.00-2.01 (m, 1H).

### Example 129: N-[(1S)-5-[2-(2-aminopyridin-3-yl)-5-(3,5-dimethylpyrazol-1-yl)imidazo[4,5-b]pyridin-3-yl]-2,3-dihydro-1H-inden-1-yl]-3-formyl-4-hydroxybenzamide

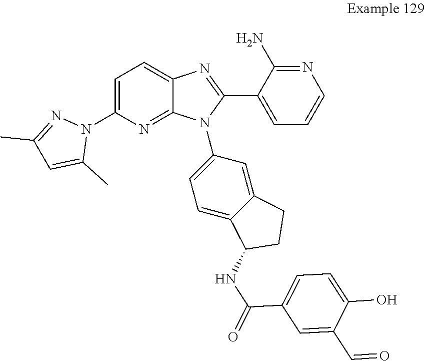 [SMILES: Cc1cc(C)n(-c2ccc3nc(-c4cccnc4N)n(-c4ccc5c(c4)CC[C@@H]5NC(=O)c4ccc(O)c(C=O)c4)c3n2)n1]

Example 129 was prepared in a manner analogous to Example 94 using 3,5-dimethyl-1H-pyrazole in place of 4-(difluoromethyl)-1H-pyrazole. MS (ESI) calculated for C₃₃H₂₈N₈O₃: 584.23 m/z, found 585.25 [M+H]⁺. ¹H NMR (400 MHz, DMSO-d₆) δ (ppm): 10.30 (s, 1H), 8.35-8.44 (m, 1H), 8.22-8.33 (m, 1H), 8.01-8.15 (m, 2H), 7.81-7.92 (m, 2H), 7.45-7.55 (m, 1H), 7.21-7.38 (m, 2H), 7.05-7.15 (m, 1H), 6.80-6.95 (m, 1H), 6.09-6.18 (m, 1H), 5.55-5.65 (m, 1H), 2.85-3.05 (m, 2H), 2.35-2.49 (m, 4H), 2.18-2.28 (m, 3H), 2.05-2.15 (m, 1H).

### Example 130: N-[(1S)-5-[2-(2-aminopyridin-3-yl)-5-(1,3-thiazol-2-yl)imidazo[4,5-b]pyridin-3-yl]-2,3-dihydro-1H-inden-1-yl]-3-fluoro-5-formyl-4-hydroxybenzamide

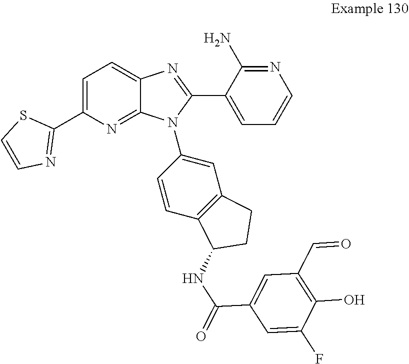 [SMILES: Nc1ncccc1-c1nc2ccc(-c3nccs3)nc2n1-c1ccc2c(c1)CC[C@@H]2NC(=O)c1cc(F)c(O)c(C=O)c1]

Example 130 was prepared in a manner analogous to Example 12 using Intermediate 57-1 in place of Intermediate 1-3 and Intermediate 44-1 in place of Intermediate 12-1. MS (ESI) calculated for C₃₁H₂₂FN₇O₃S: 591.15 m/z, found 592.15 [M+H]⁺. ¹H NMR (400 MHz, DMSO-d₆) δ (ppm): 10.29-10.32 (m, 1H), 8.39-8.43 (m, 1H), 8.20-8.23 (m, 1H), 8.15-8.20 (m, 1H), 8.05-8.13 (m, 2H), 7.95-8.01 (m, 1H), 7.75-7.80 (m, 1H), 7.60-7.69 (m, 1H), 7.40-7.51 (m, 1H), 7.31-7.38 (m, 2H), 6.65-6.76 (m, 1H), 5.57-5.69 (m, 1H), 3.02-3.11 (m, 1H), 2.86-2.98 (m, 1H), 2.54-2.59 (m, 1H), 2.01-2.18 (m, 1H). ¹⁹F NMR (400 MHz, DMSO-d₆) δ (ppm): −122.03.

### Example 131: (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-3-formyl-4-hydroxybenzamide

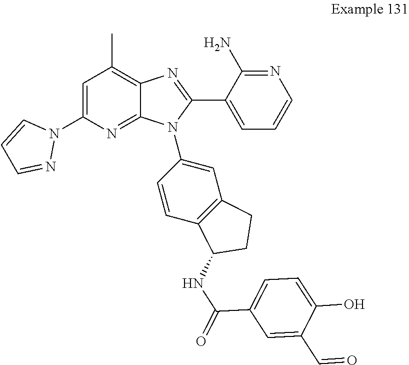 [SMILES: Cc1cc(-n2cccn2)nc2c1nc(-c1cccnc1N)n2-c1ccc2c(c1)CC[C@@H]2NC(=O)c1ccc(O)c(C=O)c1]

Example 131 was prepared in a manner analogous to Example 12 using Intermediate 131-2 in place of Intermediate 1-3 and Intermediate 57-2 in place of Intermediate 12-1. MS (ESI) calculated for C₃₂H₂₆N₈O₃: 570.21 m/z, found 571.25 [M+H]⁺. ¹H NMR (300 MHz, DMSO-d₆) δ (ppm): 10.31 (s, 1H), 8.35-8.36 (m, 2H), 8.03-8.04 (m, 2H), 7.87-7.88 (m, 1H), 7.77-7.81 (m, 1H), 7.74-7.76 (m, 1H), 7.44-7.45 (m, 1H), 7.35-7.38 (m, 2H), 7.07-7.10 (m, 1H), 6.80-6.82 (m, 1H), 6.55-6.56 (m, 1H), 5.59-5.65 (m, 1H), 3.02-3.04 (m, 1H), 2.93-2.96 (m, 1H), 2.76 (s, 3H), 2.51-2.52 (m, 1H), 2.10-2.11 (m, 1H).

### Intermediate 131-2: (S)-3-(3-(1-amino-2,3-dihydro-1H-inden-5-yl)-7-methyl-5-(1H-pyrazol-1-yl)-3H-imidazo[4,5-b]pyridin-2-yl)pyridin-2-amine

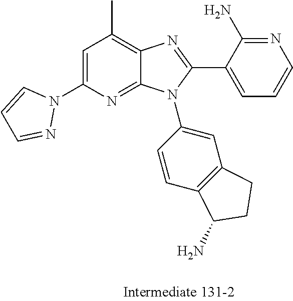 [SMILES: Cc1cc(-n2cccn2)nc2c1nc(-c1cccnc1N)n2-c1ccc2c(c1)CC[C@@H]2N]

Intermediate 131-2 was prepared in a manner analogous to Intermediate 33-1 using Intermediate 131-1 in place of Intermediate 18-2 and a reaction time 2 h instead of overnight for Step 1. MS (ESI) calculated for C₂₄H₂₂N₈: 422.20 m/z, found 423.15 [M+H]⁺.

### Intermediate 131-1: (S)—N-(5-(2-(2-aminopyridin-3-yl)-5-chloro-7-methyl-3H-imidazo[4,5-b]pyridin-3-yl)-2,3-dihydro-1H-inden-1-yl)acetamide

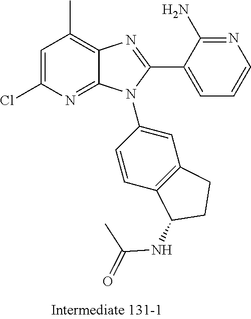 [SMILES: CC(=O)N[C@H]1CCc2cc(-n3c(-c4cccnc4N)nc4c(C)cc(Cl)nc43)ccc21]

Intermediate 131-1 was prepared in a manner analogous to Intermediate 79-1 using 2,6-dichloro-4-methyl-3-nitropyridine in place of 2,6-dichloro-3-nitropyridine. MS (ESI) calculated for C₂₃H₂₁ClN₆O: 432.15 m/z, found 433.20 [M+H]⁺.

### Example 132: (S)—N-(5-(2-(2-aminopyridin-3-yl)-5-(5-methylthiazol-2-yl)-3H-imidazo[4,5-b]pyridin-3-yl)-2,3-dihydro-1H-inden-1-yl)-3-formyl-4-hydroxybenzamide

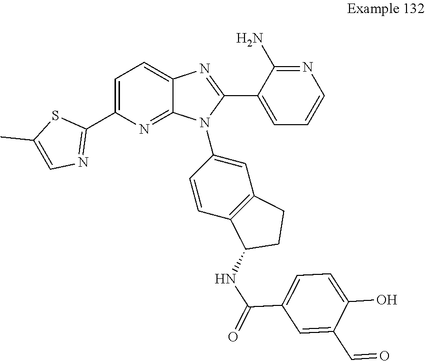 [SMILES: Cc1cnc(-c2ccc3nc(-c4cccnc4N)n(-c4ccc5c(c4)CC[C@@H]5NC(=O)c4ccc(O)c(C=O)c4)c3n2)s1]

Example 132 was prepared in a manner analogous to Example 109 using 5-methyl-2-(tributylstannyl)thiazole in place of 2-fluoro-3-(tributylstannyl)pyrazine and a reaction time of overnight instead of 2 days for Step 1. MS (ESI) calculated for C₃₂H₂₅N₇O₃S: 587.17 m/z, found 588.17 [M+H]⁺. ¹H NMR (400 MHz, DMSO-d₆) δ (ppm): 10.29 (s, 1H), 8.96-9.08 (m, 1H), 8.29-8.37 (m, 2H), 8.12-8.21 (m, 2H), 7.97-8.03 (m, 1H), 7.58-7.64 (m, 1H), 7.34-7.41 (m, 2H), 7.22-7.31 (m, 2H), 6.99-7.08 (m, 1H), 6.41-6.52 (m, 1H), 5.56-5.78 (m, 1H), 2.99-3.13 (m, 1H), 2.68-2.94 (m, 1H), 2.49-2.50 (m, 1H), 2.41-2.52 (m, 3H), 2.01-2.28 (m, 1H).

### Example 133: (S)—N-(5-(2-(2-aminopyridin-3-yl)-5-(4-methylthiazol-2-yl)-3H-imidazo[4,5-b]pyridin-3-yl)-2,3-dihydro-1H-inden-1-yl)-3-formyl-4-hydroxybenzamide

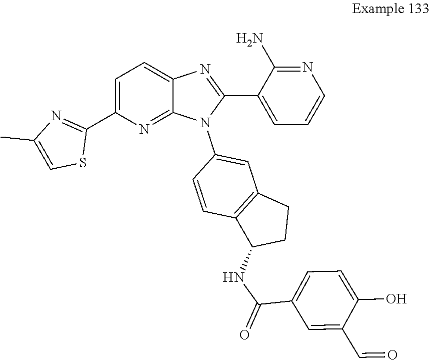 [SMILES: Cc1csc(-c2ccc3nc(-c4cccnc4N)n(-c4ccc5c(c4)CC[C@@H]5NC(=O)c4ccc(O)c(C=O)c4)c3n2)n1]

Example 133 was prepared in a manner analogous to Example 109 using 4-methyl-2-(tributylstannyl)thiazole in place of 2-fluoro-3-(tributylstannyl)pyrazine and a reaction time of overnight instead of 2 days for Step 1. MS (ESI) calculated for C₃₂H₂₅N₇O₃S: 587.17 m/z, found 588.15 [M+H]⁺. ¹H NMR (400 MHz, DMSO-d₆) δ (ppm): 10.27 (s, 1H), 8.99-9.01 (m, 1H), 8.25-8.29 (m, 2H), 8.07-8.14 (m, 2H), 8.00-8.01 (m, 1H), 7.26-7.35 (m, 5H), 7.06-7.08 (m, 1H), 6.45-6.48 (m, 1H), 5.58-5.64 (m, 1H), 2.84-3.04 (m, 2H), 2.41 (s, 3H), 2.04-2.14 (m, 1H), 1.14-1.19 (m, 1H).

### Example 134: N-[(1S)-5-[5-(3-aminopyrazol-1-yl)-2-(2-aminopyridin-3-yl)imidazo[4,5-b]pyridin-3-yl]-2,3-dihydro-1H-inden-1-yl]-3-formyl-4-hydroxybenzamide

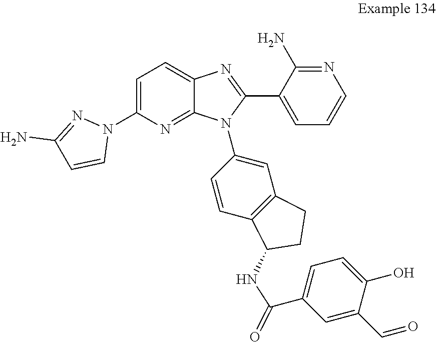 [SMILES: Nc1ccn(-c2ccc3nc(-c4cccnc4N)n(-c4ccc5c(c4)CC[C@@H]5NC(=O)c4ccc(O)c(C=O)c4)c3n2)n1]

Example 134 was prepared in a manner analogous to Example 94 using Intermediate 134-1 in place of 4-(difluoromethyl)-1H-pyrazole. MS (ESI) calculated for C₃₁H₂₅N₉O₃: 571.21 m/z, found 572.25 [M+H]⁺. ¹H NMR (400 MHz, DMSO-d₆) δ (ppm): 9.31 (s, 1H), 8.12-8.14 (m, 1H), 7.95-8.07 (m, 3H), 7.84-7.92 (m, 1H), 7.45-7.51 (m, 2H), 7.40-7.42 (m, 1H), 7.30-7.38 (m, 1H), 7.10-7.28 (m, 3H), 6.40-6.50 (m, 3H), 5.60-5.62 (m, 1H), 3.20-3.30 (m, 1H), 2.95-2.97 (m, 1H), 2.51-2.52 (m, 1H), 2.10-2.30 (m, 1H).

### Intermediate 134-1: tert-butyl (1H-pyrazol-3-yl)carbamate

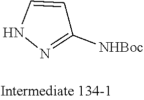 [SMILES: CC(C)(C)OC(=O)Nc1cc[nH]n1]

### Synthetic Route:

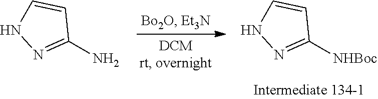 [SMILES: Nc1cc[nH]n1 ; CC(C)(C)OC(=O)Nc1cc[nH]n1]

### Step 1: Synthesis of tert-butyl N-(1H-pyrazol-3-yl)carbamate

A solution of 1H-pyrazol-3-amine (500 mg, 6.02 mmol, 1 equiv) and triethylamine (1.83 g, 18.1 mmol, 3 equiv) in dichloromethane (15 mL) was stirred overnight at room temperature. The resulting mixture was washed with water (50 mL×3), dried over anhydrous sodium sulfate, filtered, and concentrated under reduced pressure to afford tert-butyl N-(1H-pyrazol-3-yl)carbamate (Intermediate 134-1) (400 mg, 34%) as a white oil, which was used without further purification in subsequent transformations. MS (ESI) calculated for C₈H₁₃N₃O₂: 183.10 m/z, found 184.15 [M+H]⁺.

### Example 135: N-[(1S)-5-[2-(2-aminopyridin-3-yl)-5-(3-fluoropyrazol-1-yl)imidazo[4,5-b]pyridin-3-yl]-2,3-dihydro-1H-inden-1-yl]-2-fluoro-5-formyl-4-hydroxybenzamide

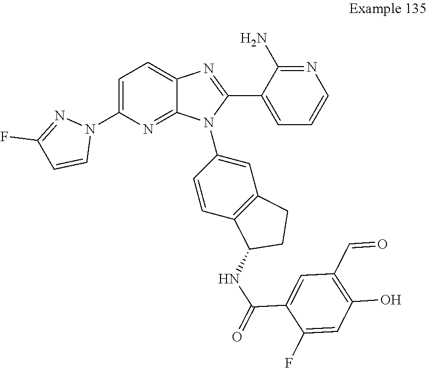 [SMILES: Nc1ncccc1-c1nc2ccc(-n3ccc(F)n3)nc2n1-c1ccc2c(c1)CC[C@@H]2NC(=O)c1cc(C=O)c(O)cc1F]

Example 135 was prepared in a manner analogous to Example 12 using Intermediate 16-1 in place of Intermediate 12-1 and Intermediate 75-1 in place of Intermediate 1-3. MS (ESI) calculated for C₃H₂₂F₂N₈O₃: 592.18 m/z, found 591.05 [M−H]⁻. ¹H NMR (300 MHz, DMSO-d₆) δ (ppm): 11.39-11.87 (m, 1H), 10.25 (s, 1H), 8.69-8.82 (m, 1H), 8.32-8.43 (m, 1H), 8.18-8.32 (m, 1H), 7.91-8.11 (m, 2H), 7.68-7.85 (m, 1H), 7.36-7.49 (m, 2H), 7.22-7.36 (m, 2H), 6.82-6.98 (m, 2H), 6.75-6.82 (m, 1H), 6.40-6.52 (m, 1H), 6.29-6.40 (m, 1H), 5.42-5.65 (m, 1H), 2.83-3.04 (m, 2H), 2.50-2.51 (m, 1H), 1.91-2.12 (m, 1H).

### Example 136: N-[(1S)-5-[2-(2-aminopyridin-3-yl)-5-chloroimidazo[4,5-b]pyridin-3-yl]-2,3-dihydro-1H-inden-1-yl]-3-formyl-4-hydroxybenzamide

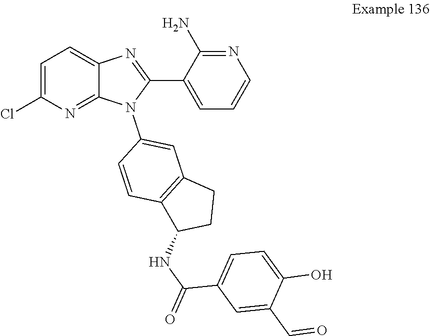 [SMILES: Nc1ncccc1-c1nc2ccc(Cl)nc2n1-c1ccc2c(c1)CC[C@@H]2NC(=O)c1ccc(O)c(C=O)c1]

Example 136 was prepared in a manner analogous to Example 94 by skipping Step 1. MS (ESI) calculated for C₂₈H₂₁ClN₆O₃: 524.14 m/z, found 525.20 [M+H]⁺. ¹H NMR (400 MHz, DMSO-d₆) δ (ppm): 10.30-10.40 (s, 1H), 8.31-8.33 (m, 2H), 8.01-8.02 (m, 2H), 7.94-8.00 (m, 1H), 7.77-7.78 (m, 1H), 7.50-7.52 (m, 1H), 7.35-7.37 (m, 1H), 7.27-7.29 (m, 1H), 7.07-7.09 (m, 1H), 6.77-6.81 (m, 1H), 5.59-5.63 (m, 1H), 3.03-3.07 (m, 1H), 2.87-3.03 (m, 1H), 2.51-2.52 (m, 1H), 2.10-2.30 (m, 1H).

### Example 137: N-[(1S)-5-[2-(2-aminopyridin-3-yl)-5-phenylimidazo[4,5-b]pyridin-3-yl]-2,3-dihydro-1H-inden-1-yl]-7-formyl-1H-1,2,3-benzotriazole-5-carboxamide

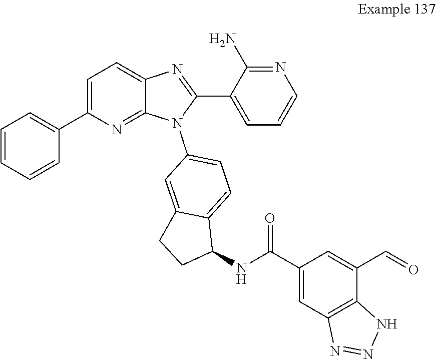 [SMILES: Nc1ncccc1-c1nc2ccc(-c3ccccc3)nc2n1-c1ccc2c(c1)CC[C@@H]2NC(=O)c1cc(C=O)c2[nH]nnc2c1]

Example 137 was prepared in a manner analogous to Example 12 using Intermediate 137-1 in place of Intermediate 12-1. MS (ESI) calculated for C₃₄H₂₅N₉O₂: 591.21 m/z, found 592.25 [M+H]⁺. ¹H NMR (400 MHz, DMSO-d₆) δ (ppm): 10.30 (s, 1H), 9.29-9.31 (m, 1H), 8.97 (s, 1H), 8.73 (s, 1H), 8.24-8.26 (m, 1H), 7.99-8.01 (m, 3H), 7.95-7.97 (m, 1H), 7.44-7.48 (m, 3H), 7.39-7.40 (m, 2H), 7.34-7.37 (m, 1H), 7.27-7.32 (m, 1H), 6.45-6.48 (m, 1H), 5.70-5.72 (m, 1H), 3.03-3.06 (m, 1H), 2.93-2.95 (m, 1H), 2.57-2.60 (m, 1H), 2.14-2.17 (m, 1H).

### Intermediate 137-1: 7-(1,3-dioxolan-2-yl)-1-((2-(trimethylsilyl)ethoxy)methyl)-1H-benzo[d][1,2,3]triazole-5-carboxylic acid

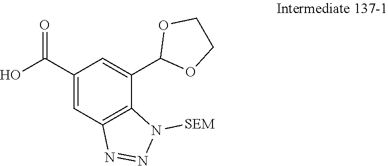 [SMILES: C[Si](C)(C)CCOCn1nnc2cc(C(=O)O)cc(C3OCCO3)c21]

### Synthetic Route:

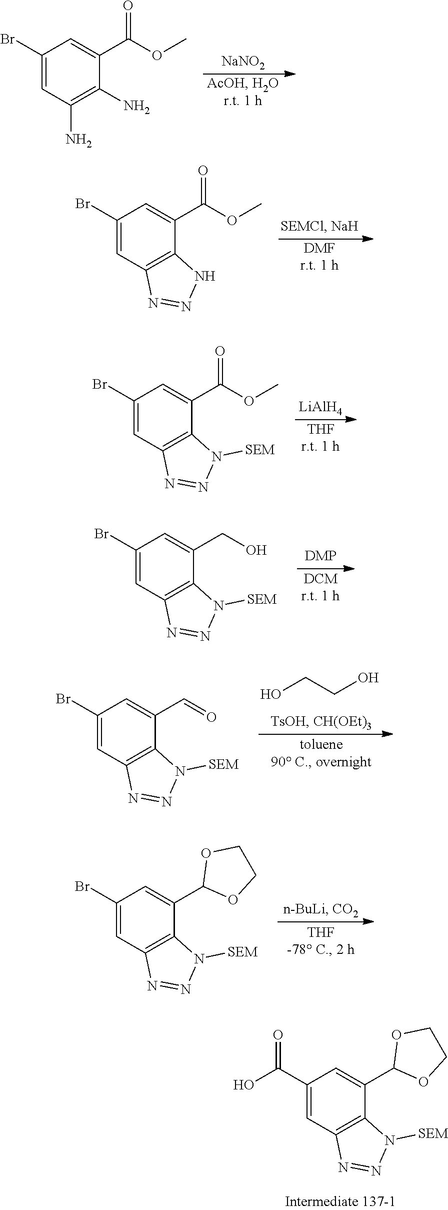

### Step 1: Synthesis of methyl 6-bromo-3H-1,2,3-benzotriazole-4-carboxylate

A solution of methyl 2,3-diamino-5-bromobenzoate (5.00 g, 20.4 mmol, 1 equiv) in acetic acid (75 mL) was stirred for 10 minutes at room temperature. A solution of sodium nitrite (1.55 g, 22.4 mmol, 1.1 equiv) in water (10 mL) was added and the reaction was stirred for 30 minutes. The precipitate was collected by filtration, rinsed with water, and dried under reduced pressure to afford methyl 6-bromo-3H-1,2,3-benzotriazole-4-carboxylate (4.1 g, 70%) as an off-white solid. MS (ESI) calculated for C₈H₆BrN₃O₂: 254.96 m/z, found 256.00 [M+H]⁺.

### Step 2: Synthesis of methyl 6-bromo-3-{[2-(trimethylsilyl)ethoxy]methyl}-1,2,3-benzotriazole-4-carboxylate

To a cooled (0° C.) solution of methyl 6-bromo-3H-1,2,3-benzotriazole-4-carboxylate (2.00 g, 7.81 mmol, 1 equiv) in N,N-dimethylformamide (50 mL) was added sodium hydride (60% dispersion, 469 mg, 11.7 mmol, 1.5 equiv). The mixture was stirred for 30 min then (2-(chloromethoxy)ethyl)trimethylsilane (2.60 g, 15.6 mmol, 2 equiv) was added and the mixture was stirred at room temperature for 1 h. The reaction was quenched by dropwise addition of water (30 mL) over 10 min. The resulting mixture was extracted with ethyl acetate (30 mL×3). The combined extracts were washed with brine (10 mL), dried over anhydrous sodium sulfate, filtered, and concentrated in vacuo. The resulting oil was then purified by silica gel column chromatography using a 0-25% gradient of ethyl acetate in petroleum ether to afford methyl 6-bromo-3-{[2-(trimethylsilyl) ethoxy]methyl}-1,2,3-benzotriazole-4-carboxylate (2 g, 65%) as a yellow oil. MS (ESI) calculated for C₁₄H₂₀BrN₃O₃Si: 385.05 m/z, found 386.05 [M+H]⁺.

### Step 3: Synthesis of (6-bromo-3-{[2-(trimethylsilyl)ethoxy]methyl}-1,2,3-benzotriazol-4-yl)methanol

To a cooled (0° C.) solution of methyl 6-bromo-3-{[2-(trimethylsilyl)ethoxy]methyl}-1,2,3-benzotriazole-4-carboxylate (2.00 g, 5.18 mmol, 1 equiv) in tetrahydrofuran (30 mL) was added lithium aluminum hydride (2.5 M, 5.18 mL, 12.9 mmol, 2.5 equiv) under nitrogen atmosphere. The resulting mixture was stirred at room temperature for 1 h. The reaction was quenched with water (100 mL). The resulting mixture was extracted with ethyl acetate (3×100 mL). The organic layers were combined, dried over anhydrous sodium sulfate, filtered, and concentrated in vacuo to afford (6-bromo-3-{[2-(trimethylsilyl)ethoxy]methyl}-1,2,3-benzotriazol-4-yl)methanol (1.8 g, 70%) as a yellow oil. MS (ESI) calculated for C₁₃H₂₀BrN₃O₂Si: 357.05 m/z, found 358.05 [M+H]⁺.

### Step 4: Synthesis of 6-bromo-3-{[2-(trimethylsilyl)ethoxy]methyl}-1,2,3-benzotriazole-4-carbaldehyde

To a solution of (6-bromo-3-{[2-(trimethylsilyl)ethoxy]methyl}-1,2,3-benzotriazol-4-yl)methanol (2.00 g, 5.58 mmol, 1 equiv) in dichloromethane (30 mL) was added Dess-Martin Periodinane (5.92 g, 14.0 mmol, 2.5 equiv). The mixture was stirred at room temperature for 1 h. The reaction was quenched with water (100 mL). The resulting mixture was extracted with ethyl acetate (3×100 mL) and the combined extracts washed with brine (30 mL), dried over anhydrous sodium sulfate, filtered, and concentrated in vacuo. The resulting oil was then purified by silica gel column chromatography using a 0-30% gradient of ethyl acetate in petroleum ether to afford 6-bromo-3-{[2-(trimethylsilyl)ethoxy]methyl}-1,2,3-benzotriazole-4-carbaldehyde (1.3 g, 63%) as a yellow oil. MS (ESI) calculated for C₁₃H₁₈BrN₃O₂Si: 355.04 m/z, found 356.00 [M+H]⁺.

### Step 5: Synthesis of N-[(1S)-5-[2-(2-aminopyridin-3-yl)-5-(1,2,4-triazol-1-yl)imidazo[4,5-b]pyridin-3-yl]-2,3-dihydro-1H-inden-1-yl]-3-formyl-4-hydroxybenzamide (Intermediate 137-1)

To a solution of 6-bromo-3-{[2-(trimethylsilyl)ethoxy]methyl}-1,2,3-benzotriazole-4-carbaldehyde (1.3 g, 3.6 mmol, 1 equiv) in toluene (50 mL) was added ethylene glycol (1.81 g, 29.2 mmol, 8 equiv), triethyl orthoformate (4.33 g, 29.2 mmol, 8 equiv) and p-toluenesulfonic acid (0.060 g, 0.37 mmol, 0.1 equiv). The resulting mixture was stirred at 90° C. overnight. The reaction mixture was then treated with water (30 mL) dropwise over 10 min. The mixture was then extracted with ethyl acetate (30 mL×2) and the combined extracts washed with brine (10 mL), dried over anhydrous sodium sulfate, filtered, and concentrated in vacuo. The resulting oil was then purified by silica gel column chromatography using a 0-20% gradient of ethyl acetate in petroleum ether to afford 5-bromo-7-(1,3-dioxolan-2-yl)-1-{[2-(trimethylsilyl)ethoxy]methyl}-1,2,3-benzotriazole (Intermediate 137-1) (800 mg, 55%) as a yellow oil. MS (ESI) calculated for C₁₅H₂₂BrN₃O₃Si: 399.06 m/z, found 400.10 [M+H]⁺.

### Example 138: (S)—N-(5-(2-(2-aminopyridin-3-yl)-5-(4-(hydroxymethyl)-1H-pyrazol-1-yl)-3H-imidazo[4,5-b]pyridin-3-yl)-2,3-dihydro-1H-inden-1-yl)-2-fluoro-5-formyl-4-hydroxybenzamide

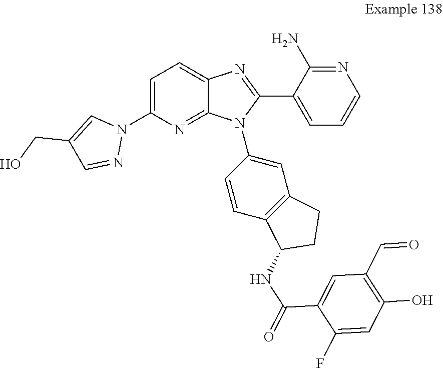 [SMILES: Nc1ncccc1-c1nc2ccc(-n3cc(CO)cn3)nc2n1-c1ccc2c(c1)CC[C@@H]2NC(=O)c1cc(C=O)c(O)cc1F]

Example 138 was prepared in a manner analogous to Example 12 using Intermediate 138-1 in place of Intermediate 1-3 and Intermediate 16-1 in place of Intermediate 12-1. MS (ESI) calculated for C₃₂H₂₅FN₈O₄: 604.20 m/z, found 605.20 [M+H]⁺. ¹H NMR (400 MHz, DMSO-d₆) δ (ppm): 10.03-10.04 (m, 1H), 8.31-8.33 (m, 1H), 8.23-8.24 (m, 1H), 7.97-7.99 (m, 3H), 7.34-7.36 (m, 1H), 7.23-7.36 (m, 4H), 6.45-6.46 (m, 1H), 6.08-6.12 (m, 1H), 5.51-5.53 (m, 1H), 4.40-4.43 (m, 2H), 2.85-2.96 (m, 2H), 2.51-2.54 (m, 1H), 1.99-2.04 (m, 1H). ¹⁹F NMR (376 MHz, DMSO-d₆) δ (ppm): −105.90.

### Intermediate 153-1: (S)-(1-(3-(1-amino-2,3-dihydro-1H-inden-5-yl)-2-(2-aminopyridin-3-yl)-3H-imidazo[4,5-b]pyridin-5-yl)-1H-pyrazol-4-yl)methanol

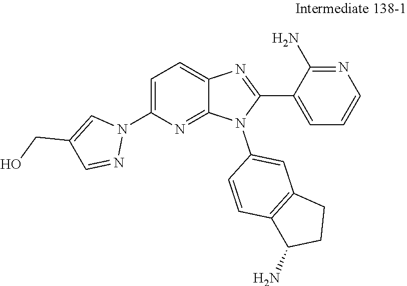 [SMILES: Nc1ncccc1-c1nc2ccc(-n3cc(CO)cn3)nc2n1-c1ccc2c(c1)CC[C@@H]2N]

Intermediate 138-1 was prepared in a manner analogous to Intermediate 33-1 using 1H-pyrazol-4-ylmethanol in place of pyrazole and a reaction time of 2 h for Step 1 instead of overnight. MS (ESI) calculated for C₂₄H₂₂N₈O: 438.19 m/z, found 439.15 [M+H]⁺.

### Example 139: N-[(1S)-5-[2-(2-aminopyridin-3-yl)-5-[4-(hydroxymethyl)pyrazol-1-yl]imidazo[4,5-b]pyridin-3-yl]-2,3-dihydro-1H-inden-1-yl]-2,3-difluoro-5-formyl-4-hydroxybenzamide

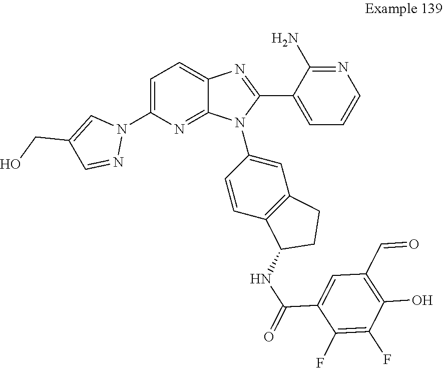 [SMILES: Nc1ncccc1-c1nc2ccc(-n3cc(CO)cn3)nc2n1-c1ccc2c(c1)CC[C@@H]2NC(=O)c1cc(C=O)c(O)c(F)c1F]

Example 139 was prepared in a manner analogous to Example 12 using Intermediate 138-1 in place of Intermediate 1-3 and Intermediate 118-1 in place of Intermediate 12-1. MS (ESI) calculated for C₃₂H₂₄F₂N₈O₄: 622.19 m/z, found 623.20 [M+H]⁺. ¹H NMR (400 MHz, DMSO-d₆) δ (ppm): 10.12 (s, 1H), 8.31-8.45 (m, 2H), 8.23-8.28 (m, 1H), 7.98-8.05 (m, 1H), 7.90-7.97 (m, 1H), 7.65-7.78 (m, 2H), 7.28-7.42 (m, 3H), 7.22-7.27 (m, 1H), 6.41-6.49 (m, 1H), 5.49-5.59 (m, 1H), 4.37-4.45 (m, 2H), 2.82-3.06 (m, 2H), 2.50-2.51 (m, 1H), 1.99-2.11 (m, 1H). ¹⁹F-NMR (400 MHz, DMSO-d₆) δ (ppm): −162.48, −162.57.

### Example 140: (S)—N-(5-(2-(2-aminopyridin-3-yl)-5-(thiazol-2-yl)-3H-imidazo[4,5-b]pyridin-3-yl)-2,3-dihydro-1H-inden-1-yl)-2,3-difluoro-5-formyl-4-hydroxybenzamide

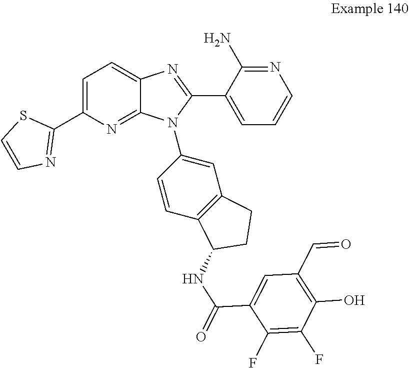 [SMILES: Nc1ncccc1-c1nc2ccc(-c3nccs3)nc2n1-c1ccc2c(c1)CC[C@@H]2NC(=O)c1cc(C=O)c(O)c(F)c1F]

Example 140 was prepared in manner analogous to Example 12 using Intermediate 57-1 in place of Intermediate 1-3 and Intermediate 118-1 in place of Intermediate 12-1. MS (ESI) calculated for C₃₁H₂₁F₂N₇O₃S: 609.14 m/z, found 610.20 [M+H]⁺. ¹H NMR (300 MHz, DMSO-d₆) δ ppm: 10.23-10.32 (m, 1H), 8.39-8.43 (m, 1H), 8.21-8.25 (m, 1H), 8.09-8.20 (m, 1H), 7.95-8.07 (m, 1H), 7.78-7.91 (m, 3H), 7.35-7.48 (m, 3H), 6.80-6.99 (m, 1H), 5.55-5.65 (m, 1H), 2.91-3.11 (m, 3H), 2.04-2.10 (m, 1H). ¹⁹F NMR (300 MHz, DMSO-d₆) δ (ppm): −128.34, −158.25.

### Example 141: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-(1,3-dioxolan-2-yl)-4-((1-methyl-1H-imidazol-2-yl)amino)benzamide

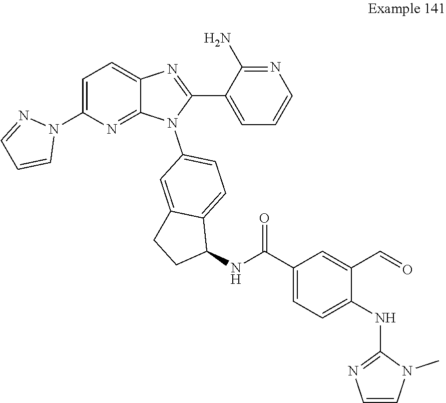 [SMILES: Cn1ccnc1Nc1ccc(C(=O)N[C@H]2CCc3cc(-n4c(-c5cccnc5N)nc5ccc(-n6cccn6)nc54)ccc32)cc1C=O]

### Synthetic Route:

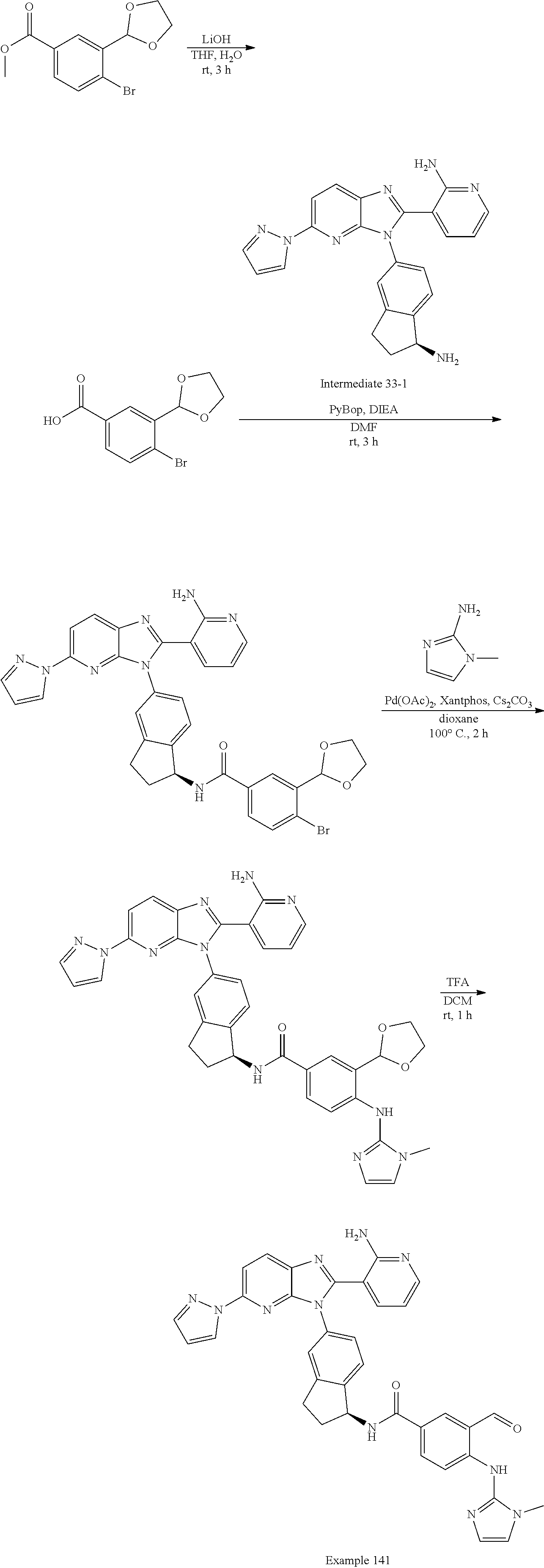

### Step 1: Synthesis of 4-bromo-3-(1,3-dioxolan-2-yl)benzoic acid

To a cooled (0° C.) solution of methyl 4-bromo-3-(1,3-dioxolan-2-yl)benzoate (900 mg, 3.14 mmol, 1 equiv) in tetrahydrofuran (15 mL) was added a solution of lithium hydroxide (225 mg, 9.40 mmol, 3 equiv) in water (2 mL) and the resulting solution was stirred overnight at room temperature. The mixture was concentrated under reduced pressure and brought to pH˜6 with 2N hydrochloric acid. The resulting precipitate was collected via filtration to provide 4-bromo-3-(1,3-dioxolan-2-yl)benzoic acid (600 mg, 70%) as a white solid, which was used in subsequent transformations without further purification. MS (ESI) calculated for C₁₀H₉BrO₄: 271.97 m/z, found 273.20 [M+H]⁺.

### Step 2: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-bromo-3-(1,3-dioxolan-2-yl)benzamide

To a solution of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (179 mg, 0.439 mmol) in N,N-dimethylformamide (2 mL) were added N,N-diisopropylethylamine (142 mg, 1.10 mmol), PyBOP (286 mg, 0.549 mmol) and 4-bromo-3-(1,3-dioxolan-2-yl)benzoic acid (100 mg, 0.366 mmol). The resulting solution was stirred at room temperature for 2 h. The mixture was purified by reverse-phase flash column chromatography on C18 silica gel using a 5-40% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-4-bromo-3-(1,3-dioxolan-2-yl)benzamide (80 mg, 33%) as a light-yellow solid. MS (ESI) calculated for C₃₃H₂₇BrN₈O₃: 662.14 m/z, found 663.00 [M+H]⁺.

### Step 3: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-(1,3-dioxolan-2-yl)-4-((1-methyl-1H-imidazol-2-yl)amino)benzamide

A mixture of N-[(1S)-5-[2-(2-aminopyridin-3-yl)-5-(pyrazol-1-yl)imidazo[4,5-b]pyridin-3-yl]-2,3-dihydro-1H-inden-1-yl]-4-bromo-3-(1,3-dioxolan-2-yl)benzamide (100 mg, 0.151 mmol), cesium carbonate (147.31 mg, 0.452 mmol), 1-methylimidazol-2-amine (18 mg, 0.18 mmol), palladium (II) acetate (3.4 mg, 0.015 mmol), and XantPhos (17.44 mg, 0.030 mmol) were suspended in 1,4-dioxane (20 mL) and the obtained solution was stirred at 100° C. for 2 h under nitrogen atmosphere. The mixture was concentrated in vacuo and the residue obtained was purified by reverse-phase flash column chromatography on C18 silica gel using a 5-40% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-3-(1,3-dioxolan-2-yl)-4-[(1-methylimidazol-2-yl)amino]benzamide as a white solid (70 mg, 68%). MS (ESI) calculated for C₃₇H₃₃N₁₁O₃: 679.28 m/z, found 680.30 [M+H]⁺.

### Step 4: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-formyl-4-((1-methyl-1H-imidazol-2-yl)amino)benzamide (Example 141)

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-3-(1,3-dioxolan-2-yl)-4-[(1-methylimidazol-2-yl)amino]benzamide (200 mg, 0.29 mmol) in dichloromethane (4.5 mL) was added 2,2,2-trifluoroacetic acid (0.5 mL) and the resulting mixture was stirred at room temperature for 1 h. The solution was concentrated in vacuo and the crude material was purified by preparative HPLC on a XSelect CSH C18 OBD Column using a 8-38% gradient of acetonitrile in water (+0.1% formic acid) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-formyl-4-((1-methyl-1H-imidazol-2-yl)amino)benzamide (Example 141) (32.1 mg, 17%). MS (ESI) calculated for C₃₅H₂₉N₁₁O₂: 635.25 m/z, found 636.60 [M+H]⁺. ¹H NMR (400 MHz, DMSO-d₆) δ (ppm): 8.38-8.44 (m, 2H), 8.14-8.16 (m, 1H), 8.06-8.08 (m, 2H), 7.99-7.02 (m, 1H), 7.83-7.90 (m, 1H), 7.60-7.68 (m, 1H), 7.31-7.47 (m, 6H), 6.80-6.84 (m, 1H), 6.74-6.75 (m, 1H), 6.56-6.57 (m, 1H), 5.64-5.67 (m, 1H), 3.70-3.75 (m, 3H), 3.04-3.06 (m, 1H), 2.93-2.95 (m, 1H), 2.50-2.52 (m, 1H), 2.12-2.13 (m, 1H).

### Example 142: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-formylbenzamide

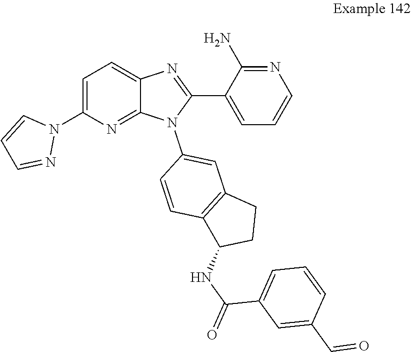 [SMILES: Nc1ncccc1-c1nc2ccc(-n3cccn3)nc2n1-c1ccc2c(c1)CC[C@@H]2NC(=O)c1cccc(C=O)c1]

Example 142 was prepared in a manner analogous to Example 12 using Intermediate 33-1 in place of Intermediate 1-3 and Intermediate 142-1 in place of Intermediate 12-1. MS (ESI) calculated for C₃₁H₂₄N₈O₂: 540.20 m/z, found 541.25 [M+H]⁺. ¹H NMR (400 MHz, DMSO-d₆) δ (ppm): 10.10 (s, 1H), 8.42-8.48 (m, 2H), 8.35-8.47 (m, 1H), 8.21-8.28 (m, 1H), 8.06-8.15 (m, 2H), 7.98-8.05 (m, 1H), 7.78-7.85 (m, 2H), 7.71-7.77 (m, 1H), 7.45-7.51 (m, 1H), 7.39-7.44 (m, 1H), 7.31-7.38 (m, 1H), 6.80-6.89 (m, 1H), 6.55-6.60 (m, 1H), 5.62-5.70 (m, 1H), 2.86-3.15 (m, 2H), 2.48-2.60 (m, 1H), 2.08-2.18 (m, 1H).

### Intermediate 142-1: 3-(1,3-dioxolan-2-yl)benzoic acid

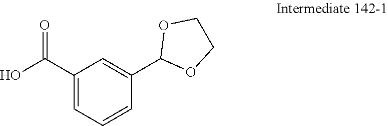 [SMILES: O=C(O)c1cccc(C2OCCO2)c1]

Intermediate 142-1 was prepared in a manner analogous to Intermediate 93-1 (starting from Step 2) using 3-formylbenzoic acid in place of 3-chloro-5-formyl-4-hydroxybenzoic acid. MS (ESI) calculated for C₁₀H₁₀O₄: 194.06 m/z, found 195.05 [M+H]⁺.

### Example 143: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-formylthiazole-4-carboxamide

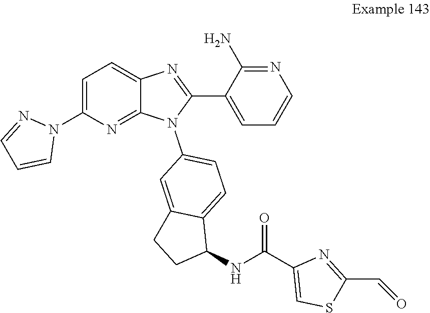 [SMILES: Nc1ncccc1-c1nc2ccc(-n3cccn3)nc2n1-c1ccc2c(c1)CC[C@@H]2NC(=O)c1csc(C=O)n1]

Example 143 was prepared in a manner analogous to Example 12 using Intermediate 33-1 in place of Intermediate 1-3 and Intermediate 143-1 in place of Intermediate 12-1. MS (ESI) calculated for C₂₈H₂₁N₉O₂S: 547.15 m/z, found 548.05 [M+H]⁺. ¹H NMR (400 MHz, DMSO-d₆) δ 9.95-9.96 (m, 1H), 8.33-8.36 (m, 2H), 8.26-8.28 (m, 1H), 8.00-8.01 (m, 1H), 7.95-7.99 (m, 1H), 7.80-7.93 (m, 1H), 7.35-7.39 (m, 2H), 7.24-7.27 (m, 2H), 6.54-6.55 (m, 1H), 6.44-6.47 (m, 1H), 5.57-5.59 (m, 1H), 3.00-3.06 (m, 1H), 2.86-2.92 (m, 1H), 2.50-2.59 (m, 1H), 2.11-2.36 (m, 1H).

### Intermediate 143-1: 2-(1,3-dioxolan-2-yl)thiazole-4-carboxylic acid

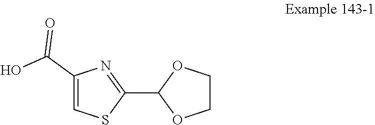 [SMILES: O=C(O)c1csc(C2OCCO2)n1]

Intermediate 143-1 was prepared in a manner analogous to Intermediate 93-1 (starting from Step 3) using methyl 2-formylthiazole-4-carboxylate in place of benzyl 4-(benzyloxy)-3-chloro-5-formylbenzoate. MS (ESI) calculated for C₇H₇NO₄S: 201.01 m/z, found 199.90 [M+H]⁻.

### Example 144: N-[(1S)-5-[2-(2-aminopyridin-3-yl)-5-(1,3-thiazol-2-yl)imidazo[4,5-b]pyridin-3-yl]-2,3-dihydro-1H-inden-1-yl]-2-fluoro-5-formyl-4-hydroxybenzamide

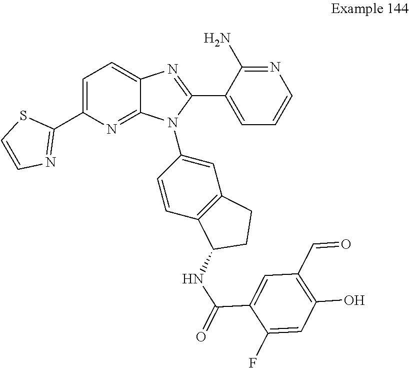 [SMILES: Nc1ncccc1-c1nc2ccc(-c3nccs3)nc2n1-c1ccc2c(c1)CC[C@@H]2NC(=O)c1cc(C=O)c(O)cc1F]

Example 144 was prepared in a manner analogous to Example 12 using Intermediate 57-1 in place of Intermediate 1-3 and Intermediate 16-1 in place of Intermediate 12-1. MS (ESI) calculated for C₃₁H₂₂FN₇O₃S: 591.15 m/z, found 592.20 [M+H]⁺. ¹H NMR (300 MHz, DMSO-d₆) δ (ppm) 10.25 (s, 1H), 8.35-8.51 (m, 1H), 8.18-8.35 (m, 1H), 8.09-8.18 (m, 1H), 7.92-8.09 (m, 3H), 7.75-7.89 (m, 1H), 7.45-7.52 (m, 1H), 7.32-7.45 (m, 2H), 6.81-7.02 (m, 2H), 5.45-5.68 (m, 1H), 2.81-3.12 (m, 2H), 2.51-2.60 (m, 1H), 2.01-2.15 (m, 1H).

### Example 145: N-[(1S)-5-[2-(2-aminopyridin-3-yl)-5-(pyrazol-1-yl)imidazo[4,5-b]pyridin-3-yl]-2,3-dihydro-1H-inden-1-yl]-2-ethynyl-5-formyl-4-hydroxybenzamide

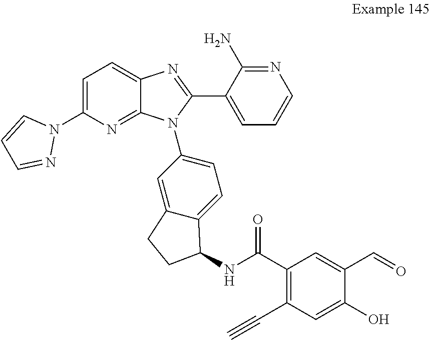 [SMILES: C#Cc1cc(O)c(C=O)cc1C(=O)N[C@H]1CCc2cc(-n3c(-c4cccnc4N)nc4ccc(-n5cccn5)nc43)ccc21]

### Synthetic Route:

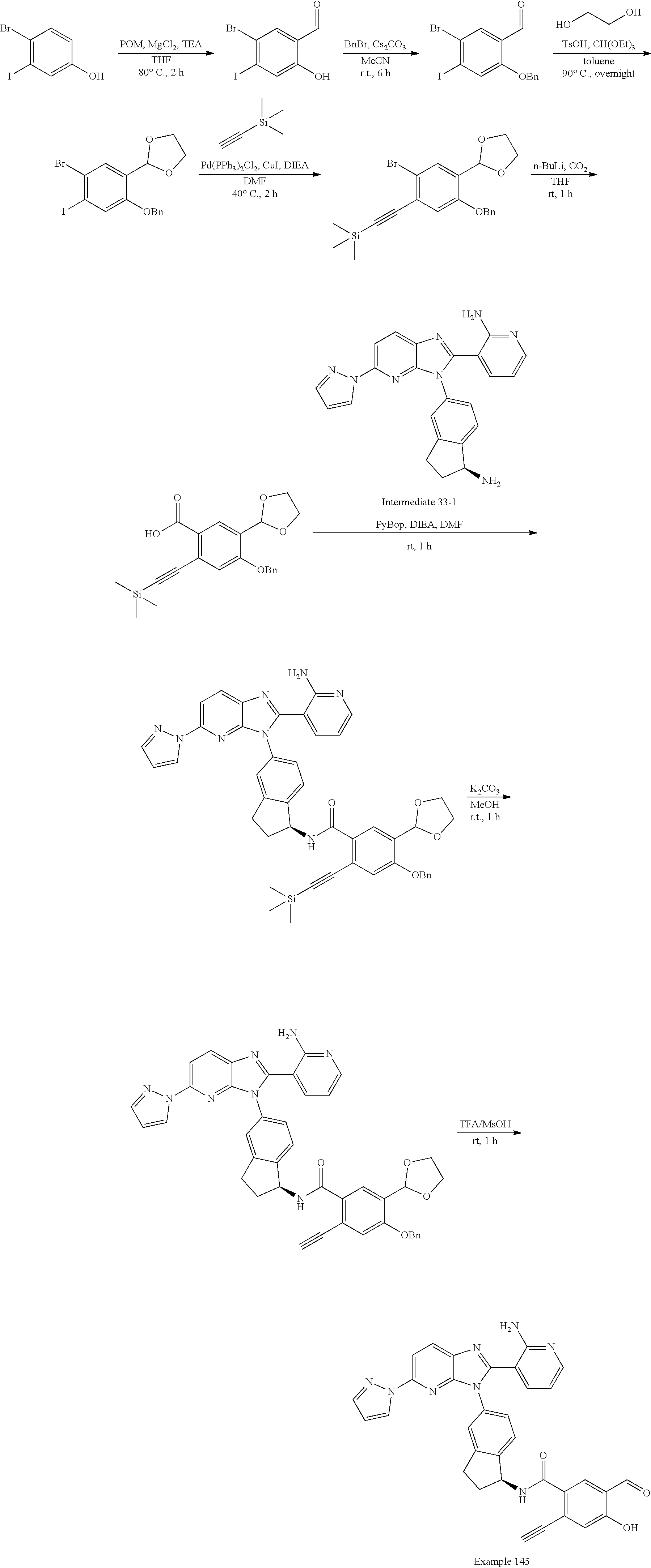

### Step 1: Synthesis of 5-bromo-2-hydroxy-4-iodobenzaldehyde

To a solution of 4-bromo-3-iodophenol (3.00 g, 10.0 mmol, 1 equiv) in tetrahydrofuran (40 mL) was added paraformaldehyde (4.52 g, 50.2 mmol, 5 equiv), magnesium (II) chloride (1.43 g, 15.1 mmol, 1.5 equiv) and triethylamine (2.54 g, 25.1 mmol, 2.5 equiv). The resulting suspension was stirred at room temperature for 10 min then at 80° C. for 2 h. The mixture was cooled to 0° C. then quenched by the addition of 2M hydrochloric acid. The resulting mixture was extracted with ethyl acetate (20 mL×3). The combined organic layers were washed with water (20 mL×3), dried over anhydrous sodium sulfate, filtered, and concentrated under reduced pressure. The residue was purified by silica gel column chromatography using a 0-30% gradient of ethyl acetate in petroleum ether to provide 5-bromo-2-hydroxy-4-iodobenzaldehyde (1 g, 30%) as a light-yellow solid. MS (ESI) calculated for C₇H₄BrIO₂: 325.84 m/z, found 326.95 [M+H]⁺.

### Step 2: Synthesis of 2-(benzyloxy)-5-bromo-4-iodobenzaldehyde

To a solution of 5-bromo-2-hydroxy-4-iodobenzaldehyde (400 mg, 1.22 mmol, 1 equiv) in acetonitrile (10 mL) was added benzyl bromide (314 mg, 1.84 mmol, 1.5 equiv) was added cesium carbonate (797 mg, 2.45 mmol, 2 equiv). The resulting mixture was stirred at room temperature for 6 h. The reaction was cooled to 0° C. and quenched by the addition of water. The mixture was extracted with ethyl acetate (20 mL×3). The combined organic layers were washed with water (10 mL×3), dried over anhydrous sodium sulfate, filtered, and concentrated under reduced pressure. The residue was triturated with petroleum ether to afford 2-(benzyloxy)-5-bromo-4-iodobenzaldehyde (300 mg, 59%) as a white solid. MS (ESI) calculated for C₁₄H₁₀BrIO₂: 415.89 m/z, found 416.90 [M+H]⁺.

### Step 3: Synthesis of 2-[2-(benzyloxy)-5-bromo-4-iodophenyl]-1,3-dioxolane

To a solution of 2-(benzyloxy)-5-bromo-4-iodobenzaldehyde (300 mg, 0.719 mmol, 1 equiv) in toluene (5 mL) were added ethylene glycol (223 mg, 3.60 mmol, 5 equiv), triethyl orthoformate (320 mg, 2.16 mmol, 3 equiv) and p-toluenesulfonic acid (12 mg, 0.072 mmol, 0.1 equiv). The resulting solution was stirred at room temperature for 10 min then overnight at 90° C. The reaction was cooled to 0° C. and quenched by the addition of saturated aqueous sodium bicarbonate. The resulting mixture was extracted with ethyl acetate (10 mL×3). The combined organic layers were washed with water (10 mL×3), dried over anhydrous sodium sulfate, filtered, and concentrated under reduced pressure. The residue was purified by silica gel column chromatography using a 0-17% gradient of ethyl acetate in petroleum ether to provide 2-[2-(benzyloxy)-5-bromo-4-iodophenyl]-1,3-dioxolane (300 mg, 90%) as a white solid. MS (ESI) calculated for C₁₆H₁₄BrIO₃: 459.92 m/z, found 460.95 [M+H]⁺.

### Step 4: Synthesis of {2-[5-(benzyloxy)-2-bromo-4-(1,3-dioxolan-2-yl)phenyl]ethynyl}trimethylsilane

To a solution of 2-[2-(benzyloxy)-5-bromo-4-iodophenyl]-1,3-dioxolane (200 mg, 0.434 mmol, 1 equiv) in N,N-dimethylformamide (5 mL) was added trimethylsilylacetylene (85 mg, 0.87 mmol, 2 equiv), bis(triphenylphosphine)palladium(II) dichloride (15 mg, 0.022 mmol, 0.05 equiv), copper (I) iodide (4.1 mg, 0.022 mmol, 0.05 equiv) and N,N-diisopropylethylamine (112 mg, 0.868 mmol, 2 equiv). The resulting mixture was stirred for 2 h at 80° C. under a nitrogen atmosphere then cooled to 0° C. and quenched by the addition of saturated aqueous ammonium chloride (20 mL). The resulting mixture was extracted with ethyl acetate (20 mL×3). The combined organic layers were washed with water (10 mL×3), dried over anhydrous sodium sulfate, filtered, and concentrated under reduced pressure. The residue was purified by silica gel column chromatography using a 0-17% gradient of ethyl acetate in petroleum ether to provide {2-[5-(benzyloxy)-2-bromo-4-(1,3-dioxolan-2-yl)phenyl]ethynyl}trimethylsilane (150 mg, 80%) as a brown solid. MS (ESI) calculated for C₂₁H₂₃BrO₃Si: 430.06 m/z, found 431.20 [M+H]⁺.

### Step 5: Synthesis of 4-(benzyloxy)-5-(1,3-dioxolan-2-yl)-2-[2-(trimethylsilyl)ethynyl]benzoic acid

To a cooled (−78° C.) solution of {2-[5-(benzyloxy)-2-bromo-4-(1,3-dioxolan-2-yl)phenyl]ethynyl}trimethylsilane (30 mg, 0.070 mmol, 1 equiv) in tetrahydrofuran (5 mL) was added n-butyllithium (2.5 M in hexane, 0.07 mL, 0.20 mmol, 2.5 equiv) and the resulting mixture was stirred for 1 h at −78° C. under a nitrogen atmosphere and for a further 1 h under carbon dioxide. The reaction was quenched by the addition of saturated aqueous ammonium chloride (10 mL) at 0° C. then concentrated under reduced pressure and purified by reverse-phase flash column chromatography on C18 silica gel using a 10-50% gradient of acetonitrile in water to provide 4-(benzyloxy)-5-(1,3-dioxolan-2-yl)-2-[2-(trimethylsilyl)ethynyl]benzoic acid (10 mg, 36%) as a light brown solid. MS (ESI) calculated for C₂₂H₂₄O₅Si: 396.14 m/z, found 397.20 [M+H]⁺.

### Step 6: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-[2-(trimethylsilyl)ethynyl]benzamide

To a suspension of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (165 mg, 0.404 mmol, 1 equiv) in N,N-dimethylformamide (2 mL) were added 4-(benzyloxy)-5-(1,3-dioxolan-2-yl)-2-[2-(trimethylsilyl)ethynyl]benzoic acid (160 mg, 0.404 mmol, 1 equiv), PyBOP (231 mg, 0.444 mmol, 1.1 equiv) and N,N-diisopropylethylamine (156 mg, 1.21 mmol, 3 equiv). The reaction mixture was stirred at room temperature for 1 h. The reaction mixture was purified by reverse-phase flash column chromatography on C18 silica gel using a 10-70% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-[2-(trimethylsilyl)ethynyl]benzamide (50 mg, 16%) as a light yellow solid. MS (ESI) calculated for C₄₅H₄₂N₈O₄Si: 786.31 m/z, found 787.30 [M+H]⁺.

### Step 7: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-ethynylbenzamide

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-[2-(trimethylsilyl)ethynyl]benzamide (50 mg, 0.064 mmol, 1 equiv) in methanol (2 mL) was added potassium carbonate (26 mg, 0.19 mmol, 3 equiv). The reaction mixture was stirred at room temperature for 1 h. The resulting mixture was filtered, rinsing with methanol (3×5 mL). The filtrate was concentrated under reduced pressure to afford N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-ethynylbenzamide (40 mg, 88%) as a light yellow solid. MS (ESI) calculated for C₄₂H₃₄N₈O₄: 714.27 m/z, found 715.30 [M+H]⁺.

### Step 8: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-2-ethynyl-5-formyl-4-hydroxybenzamide (Example 145)

To a cooled (0° C.) solution of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-3-(1,3-dioxolan-2-yl)-5-ethynylbenzamide (80 mg, 0.11 mmol, 1 equiv) in 2,2,2-trifluoroacetic acid (3 mL) was added dropwise methanesulfonic acid (1 mL). The reaction mixture was stirred at room temperature for 1 h. The residue was purified by preparative HPLC on a XSelect CSH C18 Column using a 31-61% gradient of acetonitrile in water (+0.1% formic acid) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-2-ethynyl-5-formyl-4-hydroxybenzamide (Example 145) (2.2 mg, 5%) as a light yellow solid. MS (ESI) calculated for C₃₃H₂₄N₈O₃: 580.20 m/z, found 581.25 [M+H]⁺. ¹H NMR (400 MHz, DMSO-d₆) δ (ppm): 10.31 (s, 1H), 8.38-8.43 (m, 2H), 7.99-8.06 (m, 3H), 7.83-7.84 (m, 1H), 7.45-7.52 (m, 2H), 7.19-7.28 (m, 2H), 6.63-6.65 (m, 1H), 6.56-6.58 (m, 1H), 6.05-6.26 (m, 1H), 5.44 (s, 1H), 2.98-3.22 (m, 2H), 2.41-2.47 (m, 1H), 2.33-2.35 (m, 2H).

### Example 146: (S)—N-(5-(2-(2-aminopyridin-3-yl)-5-(5-(difluoromethyl)thiazol-2-yl)-3H-imidazo[4,5-b]pyridin-3-yl)-2,3-dihydro-1H-inden-1-yl)-3-formyl-4-hydroxybenzamide

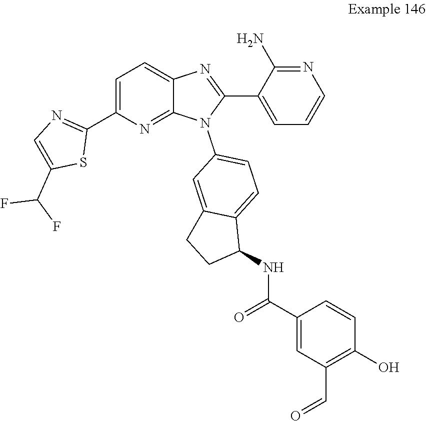 [SMILES: Nc1ncccc1-c1nc2ccc(-c3ncc(C(F)F)s3)nc2n1-c1ccc2c(c1)CC[C@@H]2NC(=O)c1ccc(O)c(C=O)c1]

Example 146 was prepared in a manner analogous to Example 121 using Intermediate 146-1 in place of 2-(tributylstannyl)-1,3-oxazole. MS (ESI) calculated for C₃₂H₂₃F₂N₇O₃S: 623.16 m/z, found 624.05 [M+H]⁺. ¹H NMR (400 MHz, DMSO-d₆) δ (ppm); 10.30 (s, 1H), 8.98-8.99 (m, 1H), 8.35-8.37 (m, 1H), 8.30-8.31 (m, 1H), 8.23-8.27 (m, 1H), 8.20-8.21 (m, 1H), 8.02-8.08 (m, 2H), 7.20-7.42 (m, 5H), 7.04-7.06 (m, 1H), 6.46-6.49 (m, 1H), 5.64-5.66 (m, 1H), 2.90-3.01 (m, 2H), 2.50-2.51 (m, 1H), 2.05-2.07 (m, 1H). ¹⁹F NMR (376 MHz, DMSO-d₆) δ (ppm): −102.31.

### Intermediate 146-1: 5-(difluoromethyl)-2-(tributylstannyl)thiazole

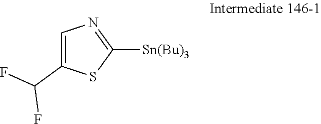

### Synthetic Route:

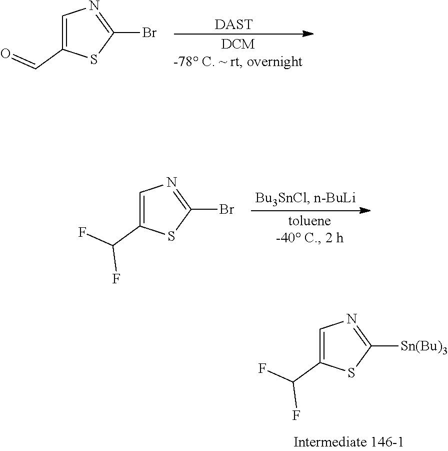

### Step 1: Synthesis of 2-bromo-5-(difluoromethyl)thiazole

To a cooled (−78° C.) solution of 2-bromothiazole-5-carbaldehyde (2.00 g, 10.4 mmol, 1 equiv) in dichloromethane (30 mL) was added diethylaminosulfur trifluoride (5.04 g, 31.2 mmol, 3 equiv) and the resulting mixture was stirred at −78° C. under nitrogen atmosphere for 3 h. The reaction was quenched with water (30 mL) and extracted with dichloromethane (2×50 mL). The combined organic layers were washed with brine, dried over anhydrous sodium sulfate, filtered, and concentrated under reduced pressure. The residue obtained was purified by silica gel column chromatography using a gradient of ethyl acetate in petroleum ether to afford 2-bromo-5-(difluoromethyl)thiazole (1.33 g, 60%) as a light-yellow oil. MS (ESI) calculated for C₁₆H₂₉F₂NSSn: 425.10 m/z, found 426.20 [M+H]⁺.

### Step 2: 5-(difluoromethyl)-2-(tributylstannyl)thiazole (Intermediate 146-1)

To a cooled (−78° C.) solution of 2-bromo-4-(difluoromethyl)thiazole (1.33 g, 6.07 mmol, 1 equiv) in toluene (30 mL) was added dropwise n-butyllithium (2.5 M in hexane, 6.07 mL, 15.185 mmol, 2.5 equiv) and the resulting mixture was stirred at −78° C. under nitrogen atmosphere for 45 min. Tributyltin chloride (2.97 g, 9.11 mmol, 1.5 equiv) was added dropwise and the mixture was stirred for another 1 h. The reaction was quenched with saturated aqueous ammonium chloride (50 mL) and the mixture was extracted with ethyl acetate (2×15 mL). The organic layers were combined, dried over sodium sulfate, filtered, and concentrated in vacuo. The resulting residue was purified by silica gel column chromatography using a gradient of ethyl acetate in petroleum ether to afford 5-(difluoromethyl)-2-(tributylstannyl)thiazole (Intermediate 146-1) (1.5 g, 58%) as a brown oil. MS (ESI) calculated for C₁₆H₂₉F₂NSSn: 425.10 m/z, found 426.05 [M+H]⁺.

### Example 147: N-[(1S)-5-[2-(2-aminopyridin-3-yl)-5-[4-(difluoromethyl)-1,3-thiazol-2-yl]imidazo[4,5-b]pyridin-3-yl]-2,3-dihydro-1H-inden-1-yl]-3-formyl-4-hydroxybenzamide

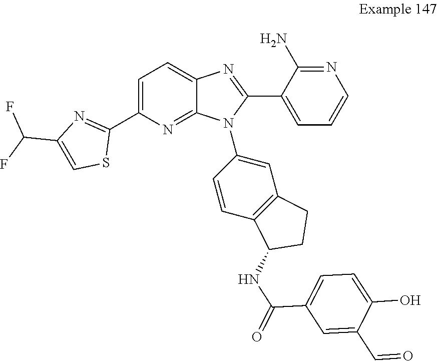 [SMILES: Nc1ncccc1-c1nc2ccc(-c3nc(C(F)F)cs3)nc2n1-c1ccc2c(c1)CC[C@@H]2NC(=O)c1ccc(O)c(C=O)c1]

Example 147 was prepared in a manner analogous to Example 121 using Intermediate 147-1 in place of 2-(tributylstannyl)-1,3-oxazole. MS (ESI) calculated for C₃₂H₂₃F₂N₇O₃S: 623.16 m/z, found 624.15 [M+H]⁺. ¹H NMR (400 MHz, DMSO-d₆) δ (ppm): 10.28 (s, 1H), 8.37-8.39 (m, 1H), 8.28-8.29 (m, 1H), 8.21-8.23 (m, 1H), 8.14 (s, 1H), 8.03-8.10 (m, 2H), 7.58-7.60 (m, 1H), 7.42 (s, 1H), 7.26-7.36 (m, 2H), 6.92-7.12 (m, 2H), 6.66-6.69 (m, 1H), 5.59-5.63 (m, 1H), 3.00-3.02 (m, 1H), 2.89-2.91 (m, 1H), 2.51 (s, 1H), 2.07-2.13 (m, 1H). ¹⁹F-NMR (400 MHz, DMSO-d₆) δ (ppm): −113.02.

### Intermediate 147-1: 4-(difluoromethyl)-2-(tributylstannyl)thiazole

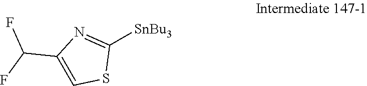

Intermediate 147-1 was prepared in a manner analogous to Intermediate 146-1 using 2-bromo-thiazole-4-carbaldehyde in place of 2-bromothiazole-5-carbaldehyde. MS (ESI) calculated for C₁₆H₂₉F₂NSSn: 425.10 m/z, found 426.05 [M+H]⁺.

### Example 148: 3-{[(1S)-5-[2-(2-aminopyridin-3-yl)-5-(pyrazol-1-yl)imidazo[4,5-b]pyridin-3-yl]-2,3-dihydro-1H-inden-1-yl]amino}-1H-indazole-7-carbaldehyde

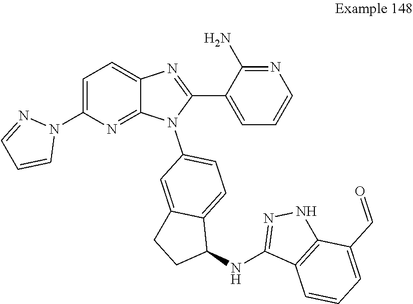 [SMILES: Nc1ncccc1-c1nc2ccc(-n3cccn3)nc2n1-c1ccc2c(c1)CC[C@@H]2Nc1n[nH]c2c(C=O)cccc12]

### Synthetic Route:

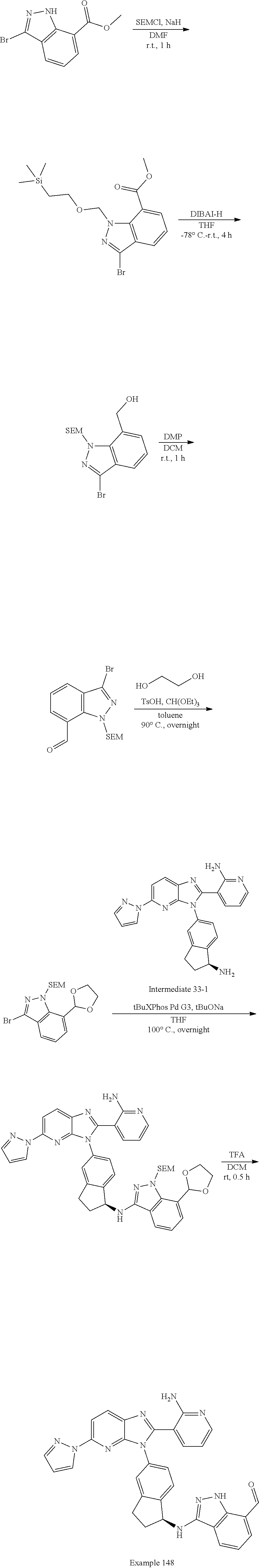

### Step 1: Synthesis of methyl 3-bromo-1-((2-(trimethylsilyl)ethoxy)methyl)-1H-indazole-7-carboxylate

To a cooled (0° C.) mixture of methyl 3-bromo-1H-indazole-7-carboxylate (2.5 g, 9.8 mmol, 1 equiv) in N,N-dimethylformamide (50 mL) was added sodium hydride (60% dispersion, 588 mg, 14.701 mmol, 1.5 equiv) in portions. The resulting mixture was stirred for 1 h at 0° C. (2-(chloromethoxy)ethyl)trimethylsilane (3.27 g, 19.6 mmol, 2 equiv) was added dropwise over 2 min and the mixture was stirred at room temperature for 2 h. The reaction mixture was cooled to 0° C. and quenched with water (50 mL). The mixture was extracted with ethyl acetate (3×100 mL) and the combined organic layers were washed with brine (2×200 mL), dried over anhydrous sodium sulfate, filtered, and concentrated under reduced pressure. The residue was purified by silica gel column chromatography, eluting with 20% ethyl acetate in petroleum ether to afford methyl 3-bromo-1-{[2-(trimethylsilyl)ethoxy]methyl}indazole-7-carboxylate (3.5 g, 93%) as a yellow oil. MS (ESI) calculated for C₁₅H₂₁BrN₂O₃Si: 384.05 m/z, found 385.05 [M+H]⁺.

### Step 2: Synthesis of (3-bromo-1-{[2-(trimethylsilyl)ethoxy]methyl}indazol-7-yl)methanol

To a cooled (−78° C.) mixture of methyl 3-bromo-1-{[2-(trimethylsilyl)ethoxy]methyl}indazole-7-carboxylate (4.00 g, 10.4 mmol, 1 equiv) in tetrahydrofuran (120 mL) was added 1M diisobutylaluminum hydride (20.8 mL, 20.8 mmol, 2 equiv) dropwise under nitrogen atmosphere. The resulting mixture was stirred for 2 h at −78° C. then for 2 h at room temperature. The reaction mixture was cooled to 0° C. and quenched with methanol. Saturated aqueous potassium sodium tartrate (100 mL) was added, and the mixture was extracted with ethyl acetate (3×300 mL). The combined organic layers were dried over anhydrous sodium sulfate, filtered, and concentrated in vacuo. The residue was purified by silica gel column chromatography, eluting with 25% ethyl acetate in petroleum ether to afford (3-bromo-1-{[2-(trimethylsilyl)ethoxy]methyl}indazol-7-yl)methanol (3.53 g, 93%) as an off-white oil. MS (ESI) calculated for C₁₄H₂₁BrN₂O₂Si: 356.06 m/z, found 357.05 [M+H]⁺.

### Step 3: Synthesis of 3-bromo-1-{[2-(trimethylsilyl)ethoxy]methyl}indazole-7-carbaldehyde

To a mixture of (3-bromo-1-{[2-(trimethylsilyl)ethoxy]methyl}indazol-7-yl)methanol (3.4 g, 9.5 mmol, 1 equiv) in dichloromethane (40 mL) was added Dess-Martin periodinane (10.09 g, 23.79 mmol, 2.5 equiv) in portions. The resulting mixture was stirred for 1 h at room temperature. Water (150 mL) was added, and the mixture was extracted with dichloromethane (3×200 mL). The combined organic layers were washed with brine (2×200 mL), dried over anhydrous magnesium sulfate, filtered, and concentrated in vacuo. The residue was purified by silica gel column chromatography, eluting with 20% ethyl acetate in petroleum ether to afford 3-bromo-1-{[2-(trimethylsilyl)ethoxy]methyl}indazole-7-carbaldehyde (2.6 g, 72%) as an off-white oil. MS (ESI) calculated for C₁₄H₁₉BrN₂O₂Si: 354.04 m/z, found 355.00 [M+H]⁺.

### Step 4: Synthesis of 3-bromo-7-(1,3-dioxolan-2-yl)-1-{[2-(trimethylsilyl)ethoxy]methyl}indazole

To a mixture of 3-bromo-1-{[2-(trimethylsilyl)ethoxy]methyl}indazole-7-carbaldehyde (1.00 g, 2.81 mmol, 1 equiv) in toluene (10 mL) were added ethylene glycol (1.40 g, 22.5 mmol, 8 equiv), triethyl orthoformate (1.26 g, 8.52 mmol, 3 equiv) and p-toluenesulfonic acid (0.050 g, 0.28 mmol, 0.1 equiv) in portions. The resulting mixture was stirred overnight at 90° C. then concentrated under reduced pressure. Water (50 mL) was added, and the mixture was extracted with ethyl acetate (3×100 mL). The combined organic layers were washed with brine (2×200 mL), dried over anhydrous sodium sulfate, filtered, and concentrated under reduced pressure. The residue was purified by silica gel column chromatography, eluting with 20% ethyl acetate in petroleum ether to afford 3-bromo-7-(1,3-dioxolan-2-yl)-1-{[2-(trimethylsilyl)ethoxy]methyl}indazole (520 mg, 46%) as a colorless oil. MS (ESI) calculated for C₁₆H₂₃BrN₂O₃Si: 398.07 m/z, found 399.05 [M+H]⁺.

### Step 5: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-7-(1,3-dioxolan-2-yl)-1-{[2-(trimethylsilyl)ethoxy]methyl}indazol-3-amine

A mixture of 3-bromo-7-(1,3-dioxolan-2-yl)-1-{[2-(trimethylsilyl)ethoxy]methyl}indazole (130 mg, 0.326 mmol, 1 equiv), 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (160 mg, 0.391 mmol, 1.2 equiv), t-BuXPhos Pd G3 (14 mg, 0.033 mmol, 0.1 equiv) and sodium tert-butoxide (62.57 mg, 0.652 mmol, 2 equiv) in tetrahydrofuran (5 mL) was stirred overnight at 100° C. under nitrogen atmosphere. The reaction was quenched with water at room temperature. The resulting mixture was extracted with ethyl acetate (100 mL×3). The combined organic layers were washed with water (100 mL×3), dried over anhydrous sodium sulfate, filtered, and concentrated under reduced pressure. The crude product was purified by reverse-phase flash column chromatography on C18 silica gel using a 20-95% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-7-(1,3-dioxolan-2-yl)-1-{[2-(trimethylsilyl)ethoxy]methyl}indazol-3-amine (100 mg, 36%) as a yellow solid. MS (ESI) calculated for C₃₉H₄₂N₁₀O₃Si: 726.32 m/z, found 727.40 [M+H]⁺.

### Step 6: Synthesis of 3-{[(1S)-5-[2-(2-aminopyridin-3-yl)-5-(pyrazol-1-yl)imidazo[4,5-b]pyridin-3-yl]-2,3-dihydro-1H-inden-1-yl]amino}-1H-indazole-7-carbaldehyde (Example 148)

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-7-(1,3-dioxolan-2-yl)-1-{[2-(trimethylsilyl)ethoxy]methyl}indazol-3-amine (80 mg, 0.11 mmol, 1 equiv) in dichloromethane (6 mL) was added 2,2,2-trifluoroacetic acid (0.6 mL) and the resulting mixture was stirred for 30 min at room temperature. The mixture was concentrated under reduced pressure and the residue was purified by preparative HPLC on a YMC Triart C18 ExRS Column using a 41-63% gradient of acetonitrile in water (+10 mmol/L ammonium bicarbonate) to afford 3-{[(1S)-5-[2-(2-aminopyridin-3-yl)-5-(pyrazol-1-yl)imidazo[4,5-b]pyridin-3-yl]-2,3-dihydro-1H-inden-1-yl]amino}-1H-indazole-7-carbaldehyde (Example 148) (10 mg, 16%) as a yellow solid. MS (ESI) calculated for C₃₁H₂₄N₁₀O: 552.21 m/z, found 553.25 [M+H]⁺. ¹H NMR (400 MHz, DMSO-d₆) δ (ppm): 10.11 (s, 1H), 8.35-8.37 (m, 2H), 8.18-8.20 (m, 1H), 8.01-8.02 (m, 1H), 7.94-7.96 (m, 2H), 7.81-7.83 (m, 1H), 7.49-7.51 (m, 1H), 7.38-7.49 (m, 1H), 7.25-7.27 (m, 2H), 7.13-7.17 (m, 1H), 6.56 (s, 1H), 6.46-6.49 (m, 1H), 5.60-5.64 (m, 1H), 3.01-3.07 (m, 1H), 2.93-2.95 (m, 1H), 2.51-2.52 (m, 1H), 2.10-2.30 (m, 1H).

### Example 149: N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-2-methylbenzamide

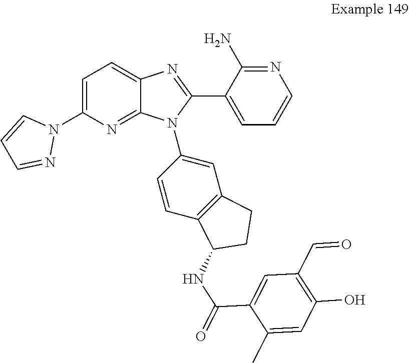 [SMILES: Cc1cc(O)c(C=O)cc1C(=O)N[C@H]1CCc2cc(-n3c(-c4cccnc4N)nc4ccc(-n5cccn5)nc43)ccc21]

Example 149 was prepared in a manner analogous to Example 7 using Intermediate 33-1 in place of Intermediate 1-2 and Intermediate 149-2 in place of Intermediate 5-1. MS (ESI) calculated for C₃₂H₂₆N₈O₃: 570.21 m/z, found 571.21 [M+H]⁺. ¹H NMR (400 MHz, DMSO-d₆) δ (ppm): 10.19 (s, 1H), 8.89-8.91 (m, 1H), 8.29-8.46 (m, 2H), 8.06-8.13 (m, 1H), 7.94-8.01 (m, 1H), 7.79-7.85 (m, 1H), 7.58-7.67 (m, 1H), 7.41-7.49 (m, 1H), 7.34-7.40 (m, 1H), 7.22-7.31 (m, 2H), 6.85-6.98 (m, 1H), 6.52-6.63 (m, 1H), 6.38-6.47 (m, 1H), 5.49-5.64 (m, 1H), 2.74-3.09 (m, 2H), 2.53-2.62 (m, 1H), 2.36-2.44 (m, 3H), 1.89-2.06 (m, 1H).

### Intermediate 149-2: 4-(benzyloxy)-5-(1,3-dioxolan-2-yl)-2-methylbenzoic acid

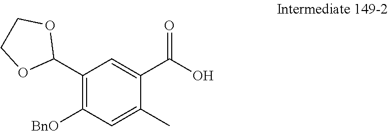 [SMILES: Cc1cc(OCc2ccccc2)c(C2OCCO2)cc1C(=O)O]

### Synthetic Route:

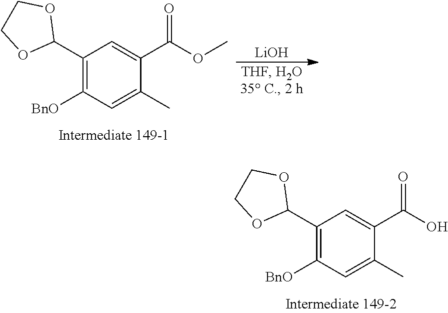

### Step 1: Synthesis of 4-(benzyloxy)-5-(1,3-dioxolan-2-yl)-2-methylbenzoic acid (Intermediate 149-2)

To a solution of methyl 4-(benzyloxy)-5-(1,3-dioxolan-2-yl)-2-methylbenzoate (Intermediate 149-1) (150 mg, 0.457 mmol) in tetrahydrofuran (4 mL) was added a solution of lithium hydroxide (44 mg, 1.8 mmol, 4 equiv) in water (1 mL) and the resulting mixture was stirred at 35° C. for 2 h. The resulting mixture was concentrated under reduced pressure to afford crude lithium 4-(benzyloxy)-5-(1,3-dioxolan-2-yl)-2-methylbenzoate (Intermediate 149-2) as a white solid (120 mg, 84% crude), which was used in subsequent transformations without further purification. MS (ESI) calculated for C₁₈H₁₈O₅: 314.12 m/z, found 315.12 [M+H]⁺.

### Intermediate 149-1: methyl 4-(benzyloxy)-5-(1,3-dioxolan-2-yl)-2-methylbenzoate

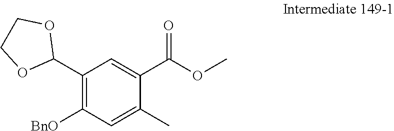 [SMILES: COC(=O)c1cc(C2OCCO2)c(OCc2ccccc2)cc1C]

### Synthetic Route:

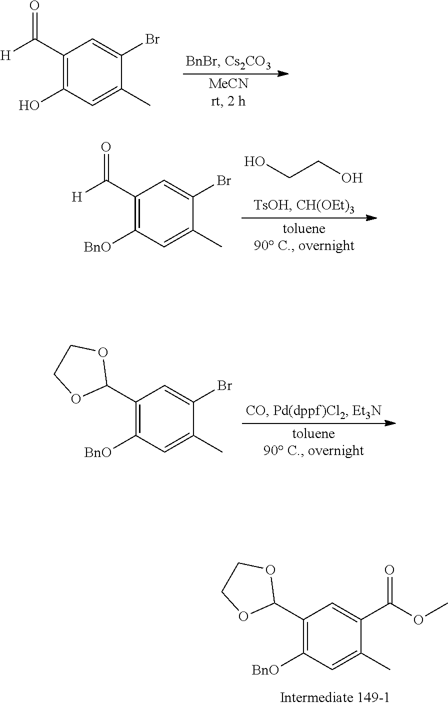

### Step 1: Synthesis of 2-(benzyloxy)-5-bromo-4-methylbenzaldehyde

A mixture of 5-bromo-2-hydroxy-4-methylbenzaldehyde (5.00 g, 23.3 mmol, 1 equiv), benzyl bromide (5.97 g, 34.9 mmol, 1.5 equiv) and cesium carbonate (15.15 g, 46.5 mmol, 2 equiv) in acetonitrile (50 mL) was stirred at room temperature for 3 h. The resulting mixture was filtered, rinsing with acetonitrile (3×10 mL). The filtrate was concentrated to dryness under reduced pressure then triturated with petroleum ether (30 mL) to afford 2-(benzyloxy)-5-bromo-4-methylbenzaldehyde (5 g, 83%) as a white solid. MS (ESI) calculated for C₁₅H₁₃BrO₂: 304.01 m/z, found 305.00 [M+H]⁺.

### Step 2: Synthesis of 2-(2-(benzyloxy)-5-bromo-4-methylphenyl)-1,3-dioxolane

A suspension of 2-(benzyloxy)-5-bromo-4-methylbenzaldehyde (3.00 g, 9.83 mmol, 1 equiv), ethylene glycol (3.05 g, 49.2 mmol, 5 equiv), p-toluenesulfonic acid (0.17 g, 0.98 mmol, 0.1 equiv) and triethyl orthoformate (4.37 g, 29.5 mmol 3 equiv) in toluene (150 mL) was stirred at 100° C. overnight. The mixture was cooled to room temperature and water (20 mL) was added. The mixture was extracted with ethyl acetate (20 mL×2). The combined organic phases were washed with brine (30 mL×3), dried with sodium sulfate, filtered, and concentrated under reduced pressure. The residue was purified by silica gel column chromatography eluting with 30% ethyl acetate in petroleum ether to afford 2-[2-(benzyloxy)-5-bromo-4-methylphenyl]-1,3-dioxolane (3.2 g, 87%) as a white solid. MS (ESI) calculated for C₁₇H₁₇BrO₃: 348.04 m/z, found 348.95, 350.95 [M+H]⁺.

### Step 3: Synthesis of methyl 4-(benzyloxy)-5-(1,3-dioxolan-2-yl)-2-methylbenzoate (Intermediate 149-1)

To a suspension of 2-[2-(benzyloxy)-5-bromo-4-methylphenyl]-1,3-dioxolane (2.00 g, 5.73 mmol) in methanol (27 mL) in a pressure tank was added [1,1′-bis(diphenylphosphino)ferrocene]dichloropalladium(II) (0.84 g, 1.1 mmol, 0.2 equiv). The vessel was purged and backfilled with nitrogen 3 times then was pressurized to 30 atm with carbon monoxide at 130° C. overnight. The reaction mixture was cooled to room temperature and filtered. The filtrate was concentrated under reduced pressure and the residue was purified by silica gel column chromatography using a 0-50% gradient of ethyl acetate in petroleum ether to afford methyl 4-(benzyloxy)-5-(1,3-dioxolan-2-yl)-2-methylbenzoate (Intermediate 149-1) (1.0 g, 49%) as a yellow solid. MS (ESI) calculated for C₁₉H₂₀O₅: 328.13 m/z, found 329.10 [M+H]⁺.

### Example 150: N-[(1S)-5-[2-(2-aminopyridin-3-yl)-5-(1,3-oxazol-2-yl)imidazo[4,5-b]pyridin-3-yl]-2,3-dihydro-1H-inden-1-yl]-2,3-difluoro-5-formyl-4-hydroxybenzamide

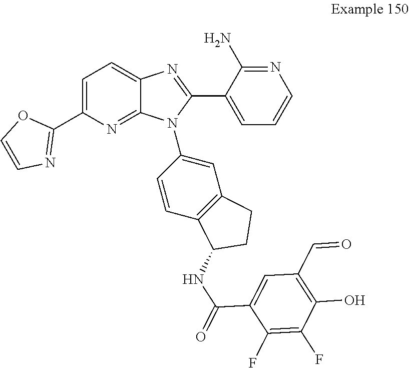 [SMILES: Nc1ncccc1-c1nc2ccc(-c3ncco3)nc2n1-c1ccc2c(c1)CC[C@@H]2NC(=O)c1cc(C=O)c(O)c(F)c1F]

Example 150 was prepared in a manner analogous to Example 12 using Intermediate 150-1 in place of Intermediate 1-3 and Intermediate 118-1 in place of Intermediate 12-1. MS (ESI) calculated for C₃₁H₂₁F₂N₇O₄: 593.16 m/z, found 594.20 [M+H]⁺. ¹H NMR (400 MHz, DMSO-d₆) δ (ppm): 10.22 (s, 1H), 8.36-8.38 (m, 1H), 8.19-8.21 (m, 2H), 8.02-8.04 (m, 1H), 7.86-7.88 (m, 1H), 7.50-7.51 (m, 1H), 7.42-7.43 (m, 3H), 7.36-7.38 (m, 1H), 6.84-6.87 (m, 1H), 5.55-5.59 (m, 1H), 2.94-3.02 (m, 2H), 2.51-2.52 (m, 1H), 2.10-2.30 (m, 1H). ¹⁹F-NMR (400 MHz, DMSO-d₆) δ (ppm): −128.29, −158.30.

### Intermediate 150-1: (S)-3-(3-(1-amino-2,3-dihydro-1H-inden-5-yl)-5-(oxazol-2-yl)-3H-imidazo[4,5-b]pyridin-2-yl)pyridin-2-amine

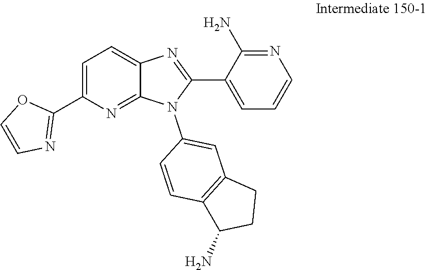 [SMILES: Nc1ncccc1-c1nc2ccc(-c3ncco3)nc2n1-c1ccc2c(c1)CC[C@@H]2N]

Intermediate 150-1 was prepared in a manner analogous to Intermediate 57-1 using 2-(tributylstannyl)-1,3-oxazole in place of 2-(tributylstannyl)-1,3-thiazole2-(tributylstannyl)-1,3-thiazole. MS (ESI) calculated for C₂₃H₁₉N₇O: 409.17 m/z, found 410.15 [M+H]⁺.

### Example 151: N-[(1S)-5-[2-(2-aminopyridin-3-yl)-5-(1,3-oxazol-2-yl)imidazo[4,5-b]pyridin-3-yl]-2,3-dihydro-1H-inden-1-yl]-2-fluoro-5-formyl-4-hydroxybenzamide

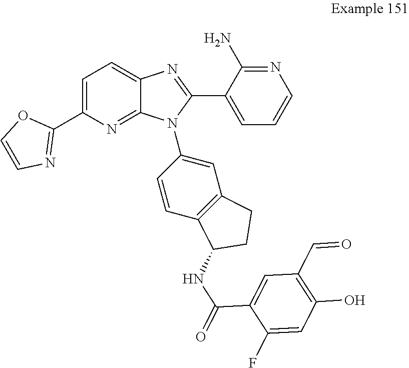 [SMILES: Nc1ncccc1-c1nc2ccc(-c3ncco3)nc2n1-c1ccc2c(c1)CC[C@@H]2NC(=O)c1cc(C=O)c(O)cc1F]

Example 151 was prepared in a manner analogous to Example 12 using Intermediate 150-1 in place of Intermediate 1-3 and Intermediate 16-1 in place of Intermediate 12-1. MS (ESI) calculated for C₃₁H₂₂FN₇O₄: 575.17 m/z, found 576.20 [M+H]⁺. ¹H NMR (400 MHz, DMSO-d₆) δ (ppm): 10.11 (s, 1H), 8.35-8.37 (m, 1H), 8.19-8.21 (m, 2H), 8.02-8.04 (m, 2H), 7.74-7.75 (m, 1H), 7.42-7.43 (m, 3H), 7.36-7.38 (m, 1H), 6.84-6.87 (m, 1H), 6.76-6.79 (m, 1H), 5.55-5.59 (m, 1H), 2.94-3.02 (m, 2H), 2.51-2.52 (m, 1H), 2.10-2.30 (m, 1H). ¹⁹F-NMR (400 MHz, DMSO-d₆) δ (ppm): −101.73.

### Example 152: (S)—N-(5-(2-(2-aminopyridin-3-yl)-5-(4-(methoxymethyl)-1H-pyrazol-1-yl)-3H-imidazo[4,5-b]pyridin-3-yl)-2,3-dihydro-1H-inden-1-yl)-2-fluoro-5-formyl-4-hydroxybenzamide

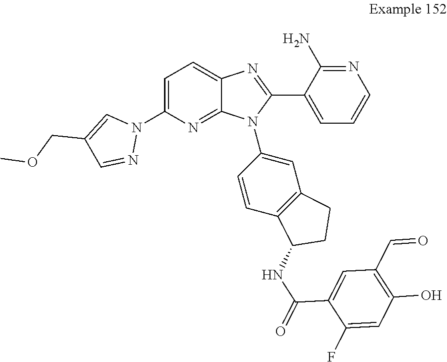 [SMILES: COCc1cnn(-c2ccc3nc(-c4cccnc4N)n(-c4ccc5c(c4)CC[C@@H]5NC(=O)c4cc(C=O)c(O)cc4F)c3n2)c1]

Example 152 was prepared in a manner analogous to Example 12 using Intermediate 152-1 in place of Intermediate 1-3 and Intermediate 16-1 in place of Intermediate 12-1. MS (ESI) calculated for C₃₃H₂₇FN₈O₄: 618.21 m/z, found 619.20 [M+H]⁺. ¹H NMR (400 MHz, DMSO-d₆) δ (ppm): 10.18 (s, 1H), 8.66-8.70 (m, 1H), 8.30-8.31 (m, 2H), 8.00-8.01 (m, 3H), 7.93-7.98 (m, 1H), 7.39-7.41 (m, 3H), 7.23-7.25 (m, 1H), 6.68-6.70 (m, 1H), 6.43-6.46 (m, 1H), 5.56-5.58 (m, 1H), 4.34-4.35 (m, 2H), 3.43-3.44 (m, 3H), 2.96-2.98 (m, 1H), 2.88-2.90 (m, 1H), 2.50-2.51 (m, 1H), 2.06-2.07 (m, 1H). ¹⁹F NMR (376 MHz, DMSO-d₆) δ (ppm): −102.72.

### Intermediate 152-1: (S)-3-(3-(1-amino-2,3-dihydro-1H-inden-5-yl)-5-(4-(methoxymethyl)-1H-pyrazol-1-yl)-3H-imidazo[4,5-b]pyridin-2-yl)pyridin-2-amine

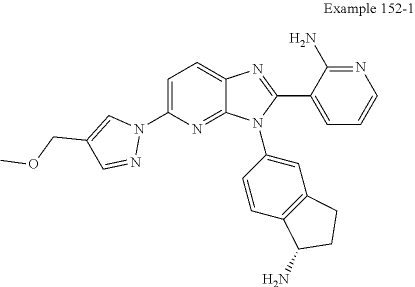 [SMILES: COCc1cnn(-c2ccc3nc(-c4cccnc4N)n(-c4ccc5c(c4)CC[C@@H]5N)c3n2)c1]

Intermediate 152-1 was prepared in a manner analogous to Intermediate 33-1 using 4-(methoxymethyl)-1H-pyrazole in place of pyrazole and a reaction time of 2 h for Step 1 instead of overnight. MS (ESI) calculated for C₂₅H₂₄N₈O: 452.21 m/z, found 453.20 [M+H]⁺.

### Example 153: (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-2,3-difluoro-5-formyl-4-hydroxybenzamide

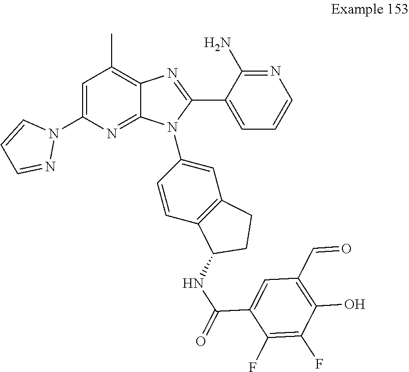 [SMILES: Cc1cc(-n2cccn2)nc2c1nc(-c1cccnc1N)n2-c1ccc2c(c1)CC[C@@H]2NC(=O)c1cc(C=O)c(O)c(F)c1F]

Example 153 was prepared in a manner analogous to Example 12 using Intermediate 131-2 in place of Intermediate 1-3 and Intermediate 118-1 in place of Intermediate 12-1. MS (ESI) calcd. for C₃₂H₂₄F₂N₈O₃: 606.19 m/z, found 607.19 [M+H]⁺. ¹H NMR (400 MHz, DMSO-d₆) δ (ppm): 10.21 (s, 1H), 8.28-8.33 (m, 1H), 7.96-8.02 (m, 1H), 7.78-7.82 (m, 3H), 7.36-7.42 (m, 3H), 7.21-7.29 (m, 1H), 6.47-6.55 (m, 2H), 5.58-5.61 (m, 1H), 2.99-3.07 (m, 1H), 2.84-2.93 (m, 1H), 2.75 (s, 3H), 2.41-2.49 (m, 1H), 1.94-2.08 (m, 1H).

### Example 154: N-[(1S)-5-[2-(2-aminopyridin-3-yl)-5-(pyrazol-1-yl)imidazo[4,5-b]pyridin-3-yl]-2,3-dihydro-1H-inden-1-yl]-4-[(dimethylsulfamoyl)amino]-3-formylbenzamide

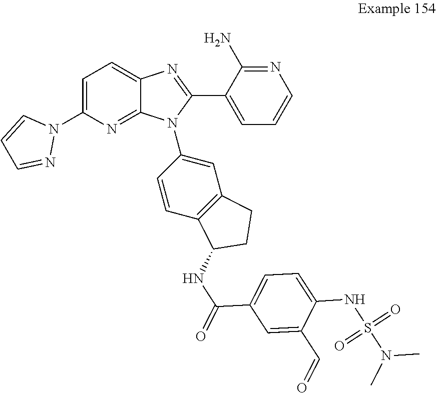 [SMILES: CN(C)S(=O)(=O)Nc1ccc(C(=O)N[C@H]2CCc3cc(-n4c(-c5cccnc5N)nc5ccc(-n6cccn6)nc54)ccc32)cc1C=O]

Example 154 was prepared in a manner analogous to Example 12 using Intermediate 33-1 in place of Intermediate 1-3 and Intermediate 154-1 in place of Intermediate 12-1. MS (ESI) calcd. for C₃₃H₃₀N₁₀O₄S: 662.22 m/z, found 663.25 [M+H]⁺. ¹HNMR (400 MHz, DMSO-d₆) δ (ppm): 10.01 (s, 1H), 8.39-8.42 (m, 2H), 8.35-8.36 (m, 1H), 8.16-8.19 (m, 1H), 7.98-8.02 (m, 2H), 7.77-7.80 (m, 2H), 7.58-7.60 (m, 1H), 7.44 (s, 1H), 7.38-7.40 (m, 1H), 7.31-7.33 (m, 1H), 6.79-6.83 (m, 1H), 6.55-6.56 (m, 1H), 5.58-5.62 (m, 1H), 3.02-3.08 (m, 1H), 2.89-2.95 (m, 1H), 2.74-2.76 (m, 6H), 2.54-2.58 (m, 1H), 2.09-2.11 (m, 1H).

### Intermediate 154-1: 4-((N,N-dimethylsulfamoyl)amino)-3-(1,3-dioxolan-2-yl)benzoic acid

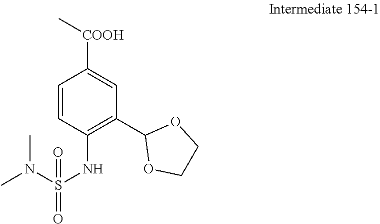

### Synthetic Route:

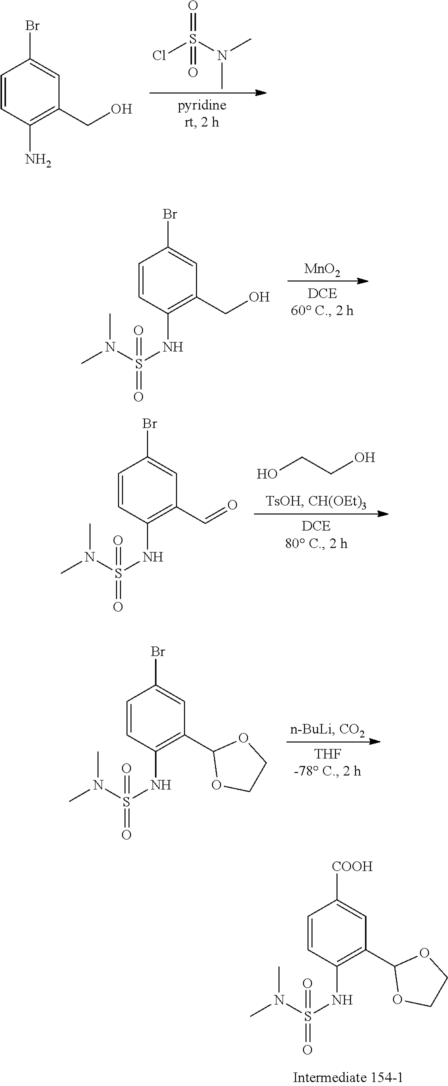

### Step 1: Synthesis of {5-bromo-2-[(dimethylsulfamoyl)amino]phenyl}methanol

To a solution of (2-amino-5-bromophenyl)methanol (2.00 g, 9.90 mmol, 1 equiv) in pyridine (20 mL) was added dimethylsulphamoyl-chloride (1.42 g, 9.90 mmol, 1 equiv). The resulting mixture was stirred at room temperature for 2 h then the solvent was removed under reduced pressure. The resulting mixture was purified by reverse-phase flash column chromatography on C18 silica gel using a 20-70% gradient of acetonitrile in water (+0.05% ammonium bicarbonate) to afford {5-bromo-2-[(dimethylsulfamoyl)amino]phenyl}methanol (1.7 g, 56%) as a yellow oil. MS (ESI) calcd. for C₉H₁₃BrN₂O₃S: 307.98 m/z, found 306.85 [M−H]⁻.

### Step 2: Synthesis of 5-bromo-2-[(dimethylsulfamoyl)amino]benzaldehyde

To a solution of {5-bromo-2-[(dimethylsulfamoyl)amino]phenyl}methanol (1.00 g, 3.23 mmol, 1 equiv) in 1,2-dichloroethane (20 mL) was added manganese (IV) oxide (2.81 g, 32.3 mmol, 10 equiv) and the resulting mixture was stirred at 60° C. for 2 h. The mixture was filtered, rinsing with ethyl acetate (10 mL). The filtrate was concentrated under reduced pressure to afford 5-bromo-2-[(dimethylsulfamoyl)amino]benzaldehyde (850 mg, 86%) as a yellow solid, which was used without further purification in the next step. MS (ESI) calcd. for C₉H₁₁BrN₂O₃S: 305.97 m/z, found 304.85 [M−H]⁻.

### Step 3: Synthesis of {[4-bromo-2-(1,3-dioxolan-2-yl)phenyl]sulfamoyl}dimethylamine

To a solution of 5-bromo-2-[(dimethylsulfamoyl)amino]benzaldehyde (850 mg, 2.77 mmol, 1 equiv) in 1,2-dichloroethane (20 mL) was added ethylene glycol (859 mg, 13.8 mmol, 5 equiv), p-toluenesulfonic acid (238 mg, 1.38 mmol, 0.5 equiv) and triethyl orthoformate (8.31 mmol, 1.23 g, 3 equiv) and the resulting mixture was stirred at 80° C. for 2 h. The solvent was removed by distillation under vacuum and the residue was purified by silica gel column chromatography using a 0-10% gradient of ethyl acetate in petroleum ether to afford {[4-bromo-2-(1,3-dioxolan-2-yl)phenyl]sulfamoyl}dimethylamine (770 mg, 79%) as a yellow oil. MS (ESI) calcd. for C₁₁H₁₅BrN₂O₄S: 349.99 m/z, found 348.90 [M−H]⁻.

### Step 4: Synthesis of 4-[(dimethylsulfamoyl)amino]-3-(1,3-dioxolan-2-yl)benzoic acid (Intermediate 154-1)

To a cooled (−78° C.) solution of {[4-bromo-2-(1,3-dioxolan-2-yl)phenyl]sulfamoyl}dimethylamine (350 mg, 0.997 mmol, 1 equiv) in tetrahydrofuran (10 mL) was added dropwise n-butyllithium solution (2.5 M in hexane, 1 mL, 2.49 mmol, 2.5 equiv) under nitrogen atmosphere. The reaction mixture was stirred at −78° C. for 30 mins then under carbon dioxide for 30 min. The reaction was quenched with saturated aqueous ammonium chloride (10 mL) and the mixture was extracted with ethyl acetate (2×30 mL). The combined organic extracts were washed with brine (10 mL), dried over anhydrous sodium sulfate, filtered, and concentrated under reduced pressure to afforded 4-[(dimethylsulfamoyl)amino]-3-(1,3-dioxolan-2-yl)benzoic acid (Intermediate 154-1) (370 mg, crude quant) as a white solid, which was used without further purification in subsequent transformations. MS (ESI) calcd. for C₁₂H₁₆N₂O₆S: 316.07 m/z, found 315.00 [M−H]⁻.

### Example 155: N-[(1S)-5-[2-(2-aminopyridin-3-yl)-5-(pyrazol-1-yl)imidazo[4,5-b]pyridin-3-yl]-2,3-dihydro-1H-inden-1-yl]-4-ethynyl-3-formylbenzamide

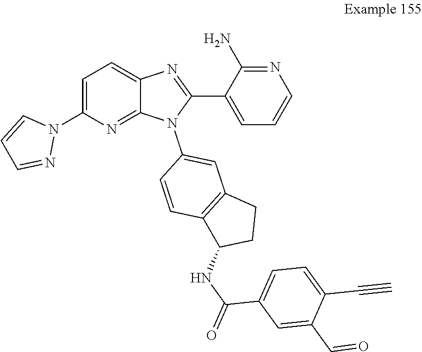 [SMILES: C#Cc1ccc(C(=O)N[C@H]2CCc3cc(-n4c(-c5cccnc5N)nc5ccc(-n6cccn6)nc54)ccc32)cc1C=O]

Example 155 was prepared in a manner analogous to Example 5 using Intermediate 33-1 in place of Intermediate 1-3 and Intermediate 155-1 in place of Intermediate 5-1. MS (ESI) calcd. for C₃₃H₂₄N₈O₂: 564.20 m/z, found 565.25 [M+H]⁺. ¹H NMR (400 MHz, DMSO-d₆) δ (ppm): 10.41 (s, 1H), 9.28-9.30 (m, 1H), 8.35-8.40 (m, 3H), 8.22-8.23 (m, 1H), 8.20-8.21 (m, 1H), 8.02-8.03 (m, 1H), 7.81-7.82 (m, 2H), 7.31-7.39 (m, 2H), 7.28-7.30 (m, 2H), 6.56-6.57 (m, 1H), 6.48-6.50 (m, 1H), 5.64-5.66 (m, 1H), 4.77 (s, 1H), 2.94-3.06 (m, 2H), 2.92-2.94 (m, 1H), 2.08-2.14 (m, 1H).

### Intermediate 155-1: 3-(1,3-dioxolan-2-yl)-4-ethynylbenzoic acid

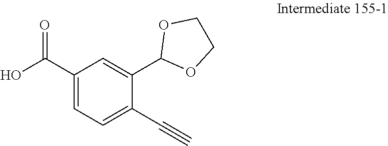 [SMILES: C#Cc1ccc(C(=O)O)cc1C1OCCO1]

Intermediate 155-1 was prepared in a manner analogous to Intermediate 112-1 (starting from Step 3) using methyl 4-bromo-3-formylbenzoate in place of benzyl 4-(benzyloxy)-2-bromo-3-formylbenzoate. MS (ESI) calcd. for C₁₂H₁₀O₄: 218.06 m/z, found 217.10 [M−H]⁻.

### Example 156: (S)—N-(5-(2-(2-aminopyridin-3-yl)-5-(3-(azetidin-1-yl)-1H-pyrazol-1-yl)-3H-imidazo[4,5-b]pyridin-3-yl)-2,3-dihydro-1H-inden-1-yl)-3-formyl-4-hydroxybenzamide

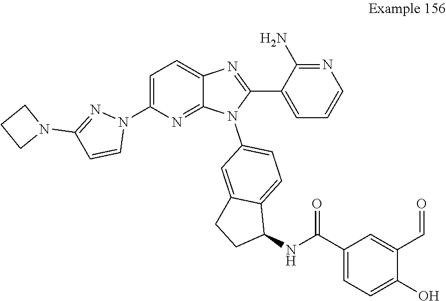 [SMILES: Nc1ncccc1-c1nc2ccc(-n3ccc(N4CCC4)n3)nc2n1-c1ccc2c(c1)CC[C@@H]2NC(=O)c1ccc(O)c(C=O)c1]

### Synthetic Route:

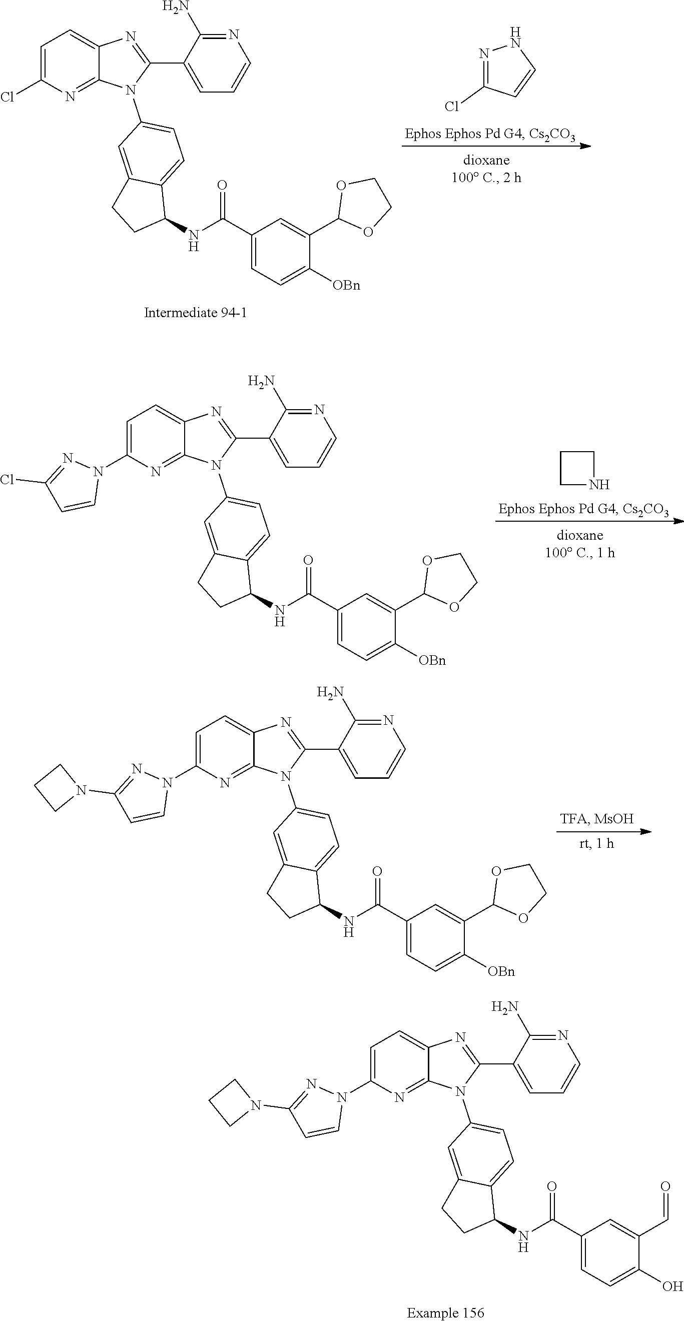

### Step 1: Synthesis of (S)—N-(5-(2-(2-aminopyridin-3-yl)-5-(3-chloro-1H-pyrazol-1-yl)-3H-imidazo[4,5-b]pyridin-3-yl)-2,3-dihydro-1H-inden-1-yl)-4-(benzyloxy)-3-(1,3-dioxolan-2-yl)benzamide

To a solution of (S)—N-(5-(2-(2-aminopyridin-3-yl)-5-chloro-3H-imidazo[4,5-b]pyridin-3-yl)-2,3-dihydro-1H-inden-1-yl)-4-(benzyloxy)-3-(1,3-dioxolan-2-yl)benzamide (Intermediate 94-1) (200 mg, 0.303 mmol, 1 equiv) and 3-chloro-1H-pyrazole (62 mg, 0.61 mmol, 2 equiv) in 1,4-dioxane (5 mL) were added EPhos (32 mg, 0.061 mmol, 0.1 equiv), EPhos Pd G4 (56 mg, 0.061 mmol, 0.1 equiv) and cesium carbonate (297 mg, 0.909 mmol, 3 equiv). The mixture was stirred for 1 h at 100° C. under nitrogen atmosphere. The mixture was allowed to cool to room temperature and water (50 mL) was added. The resulting mixture was extracted with ethyl acetate (3×50 mL). The organic layers were combined, dried over anhydrous sodium sulfate, filtered, and concentrated. The residue was purified by reverse-phase flash column chromatography on C18 silica gel using a 10-50% gradient of acetonitrile in water (+10 mmol/L ammonium bicarbonate) to afford N-[(1S)-5-[2-(2-aminopyridin-3-yl)-5-(3-chloropyrazol-1-yl) imidazo[4,5-b]pyridin-3-yl]-2,3-dihydro-1H-inden-1-yl]-4-(benzyloxy)-3-(1,3-dioxolan-2-yl)benzamide (90 mg, 37%) as a yellow solid. MS (ESI) calcd. for C₄₀H₃₃ClN₈O₄: 724.23 m/z, found 725.25 [M+H]⁺.

### Step 2: Synthesis of (S)—N-(5-(2-(2-aminopyridin-3-yl)-5-(3-(azetidin-1-yl)-1H-pyrazol-1-yl)-3H-imidazo[4,5-b]pyridin-3-yl)-2,3-dihydro-1H-inden-1-yl)-4-(benzyloxy)-3-(1,3-dioxolan-2-yl)benzamide

A mixture of cesium carbonate (202 mg, 0.620 mmol, 5 equiv), azetidine (142 mg, 2.48 mmol, 2 equiv), EPhos (6.6 mg, 0.012 mmol, 0.1 equiv), EPhos Pd G4 (11 mg, 0.012 mmol, 0.1 equiv) and (S)—N-(5-(2-(2-aminopyridin-3-yl)-5-(3-chloro-1H-pyrazol-1-yl)-3H-imidazo[4,5-b]pyridin-3-yl)-2,3-dihydro-1H-inden-1-yl)-4-(benzyloxy)-3-(1,3-dioxolan-2-yl)benzamide (90 mg, 0.12 mmol) in 1,4-dioxane (5 mL) was stirred for 1 h at 100° C. under a nitrogen atmosphere. The mixture was allowed to cool to room temperature and water (50 mL) was added. The resulting mixture was extracted with ethyl acetate (3×50 mL). The organic layers were combined, dried over anhydrous sodium sulfate, filtered, and concentrated. The residue was purified by reverse-phase flash column chromatography on C18 silica gel using a 10-50% gradient of acetonitrile in water (+10 mmol/L ammonium bicarbonate) to afford (S)—N-(5-(2-(2-aminopyridin-3-yl)-5-(3-(azetidin-1-yl)-1H-pyrazol-1-yl)-3H-imidazo[4,5-b]pyridin-3-yl)-2,3-dihydro-1H-inden-1-yl)-4-(benzyloxy)-3-(1,3-dioxolan-2-yl)benzamide (60 mg, 53%) as a yellow solid. MS (ESI) calcd. for C₄₃H₃₉N₉O₄: 745.31 m/z, found 746.35 [M+H]⁺.

### Step 3: Synthesis of (S)—N-(5-(2-(2-aminopyridin-3-yl)-5-(3-(azetidin-1-yl)-1H-pyrazol-1-yl)-3H-imidazo[4,5-b]pyridin-3-yl)-2,3-dihydro-1H-inden-1-yl)-3-formyl-4-hydroxybenzamide (Example 156)

To a solution of (S)—N-(5-(2-(2-aminopyridin-3-yl)-5-(3-(azetidin-1-yl)-1H-pyrazol-1-yl)-3H-imidazo[4,5-b]pyridin-3-yl)-2,3-dihydro-1H-inden-1-yl)-4-(benzyloxy)-3-(1,3-dioxolan-2-yl)benzamide (60 mg, 0.080 mmol) in 2,2,2-trifluoroacetic acid (1.5 mL) was added methanesulfonic acid (0.3 mL). The reaction mixture was stirred at room temperature for 1 h. After concentration, the mixture was brought to pH˜8 with saturated aqueous sodium bicarbonate. The resulting mixture was concentrated under reduced pressure and the crude product was purified by preparative HPLC on a XBridge Prep OBD Column using a 10-40% gradient of acetonitrile in water (+10 mmol/L ammonium bicarbonate) to afford (S)—N-(5-(2-(2-aminopyridin-3-yl)-5-(3-(azetidin-1-yl)-1H-pyrazol-1-yl)-3H-imidazo[4,5-b]pyridin-3-yl)-2,3-dihydro-1H-inden-1-yl)-3-formyl-4-hydroxybenzamide (Example 156) (7.5 mg, 15%) as a yellow solid. MS (ESI) calcd. for C₃₄H₂₉N₉O₃: 611.24 m/z, found 612.25 [M+H]⁺. ¹H NMR (400 MHz, DMSO-d₆) δ (ppm): 10.21 (s, 1H), 8.17-8.24 (m, 2H), 8.07-8.08 (m, 1H), 7.93-7.97 (m, 2H), 7.69-7.71 (m, 1H), 7.30-7.32 (m, 2H), 7.21-7.24 (m, 2H), 6.86-6.87 (m, 1H), 6.43-6.47 (m, 1H), 8.84-8.85 (m, 1H), 5.56-5.60 (m, 1H), 3.82-3.86 (m, 4H), 3.00-3.03 (m, 1H), 2.84-2.90 (m, 1H), 2.50-2.52 (m, 1H), 2.26-2.34 (m, 2H), 2.03-2.08 (m, 1H).

### Example 157: (S)—N-(5-(2-(2-aminopyridin-3-yl)-5-(1-(difluoromethyl)-1H-pyrazol-3-yl)-3H-imidazo[4,5-b]pyridin-3-yl)-2,3-dihydro-1H-inden-1-yl)-2,3-difluoro-5-formyl-4-hydroxybenzamide

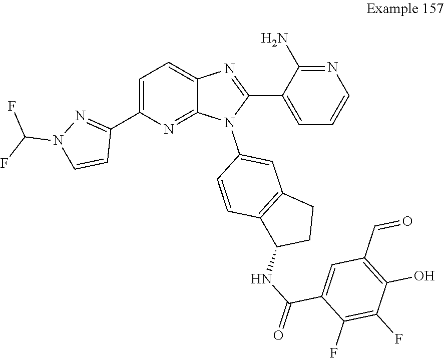 [SMILES: Nc1ncccc1-c1nc2ccc(-c3ccn(C(F)F)n3)nc2n1-c1ccc2c(c1)CC[C@@H]2NC(=O)c1cc(C=O)c(O)c(F)c1F]

Example 157 was prepared in a manner analogous to Example 19 using Intermediate 79-2 in place of Intermediate 18-2, 1-(difluoromethyl)-3-(4,4,5,5-tetramethyl-1,3,2-dioxaborolan-2-yl)pyrazole in place of 3-(4-methylpiperazin-1-yl)phenylboronic acid, 4N hydrochloric acid in 1,4-dioxane at room temperature for 1 h instead of hydrochloric acid in methanol at 90° C. overnight, PyBOP in place of HATU and Intermediate 118-1 in place of Intermediate 5-1. MS (ESI) calcd. for C₃₂H₂₂F₄N₈O₃: 642.18 m/z, found 643.20 [M+H]⁺. ¹H NMR (300 MHz, DMSO-d₆) δ (ppm): 10.05 (s, 1H), 8.25-8.29 (m, 2H), 8.00-8.06 (m, 2H), 7.64-7.81 (m, 2H), 7.27-7.39 (m, 4H), 6.91-6.92 (m, 1H), 6.47-6.51 (m, 1H), 5.54-5.61 (m, 1H), 2.90-3.08 (m, 1H), 2.75-2.77 (m, 1H), 2.53-2.54 (m, 1H), 2.30-2.31 (m, 1H). ¹⁹F-NMR (400 MHz, DMSO-d₆) δ (ppm): −94.15, −135.02, −163.31.

### Example 158: N-[(1S)-5-[2-(2-aminopyridin-3-yl)-5-[1-(difluoromethyl)pyrazol-3-yl]imidazo[4,5-b]pyridin-3-yl]-2,3-dihydro-1H-inden-1-yl]-2-fluoro-5-formyl-4-hydroxybenzamide

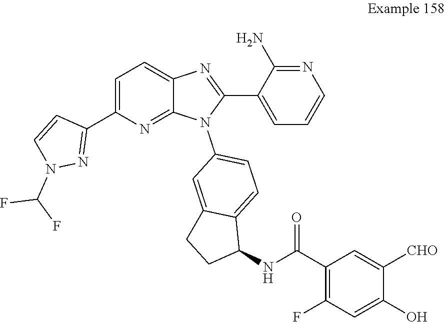 [SMILES: Nc1ncccc1-c1nc2ccc(-c3ccn(C(F)F)n3)nc2n1-c1ccc2c(c1)CC[C@@H]2NC(=O)c1cc(C=O)c(O)cc1F]

Example 158 was prepared in a manner analogous to Example 113 using Intermediate 158-1 in place of Intermediate 94-1. MS (ESI) calcd. for C₃₂H₂₃F₃N₈O₃: 624.18 m/z, found 625.25 [M+H]⁺. ¹H NMR (300 MHz, DMSO-d₆) δ (ppm): 10.20 (s, 1H), 8.58-8.81 (m, 1H), 8.17-8.38 (m, 2H), 7.62-8.15 (m, 4H), 7.35-7.51 (m, 2H), 7.19-7.35 (m, 2H), 6.85-7.03 (m, 1H), 6.58-6.79 (m, 1H), 6.35-6.55 (m, 1H), 5.43-5.68 (m, 1H), 2.81-3.15 (m, 2H), 2.51-2.60 (m, 1H), 1.93-2.15 (m, 1H).

### Intermediate 158-1: (S)—N-(5-(2-(2-aminopyridin-3-yl)-5-chloro-3H-imidazo[4,5-b]pyridin-3-yl)-2,3-dihydro-1H-inden-1-yl)-2-fluoro-5-formyl-4-hydroxybenzamide

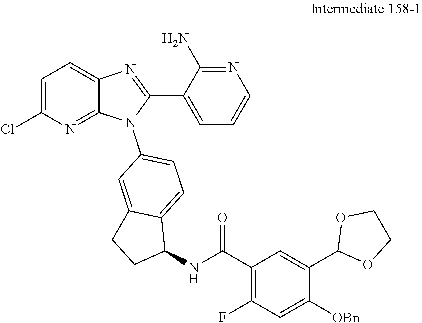 [SMILES: Nc1ncccc1-c1nc2ccc(Cl)nc2n1-c1ccc2c(c1)CC[C@@H]2NC(=O)c1cc(C2OCCO2)c(OCc2ccccc2)cc1F]

Intermediate 158-1 was prepared in a manner analogous to Intermediate 79-1 using PyBOP in place of HATU and Intermediate 16-1 in place of Intermediate 57-2. MS (ESI) calcd. for C₃₇H₃₀ClFN₆O₄: 676.20 m/z, found 677.25 [M+H]⁺.

### Example 159: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-2,4-dihydroxybenzamide

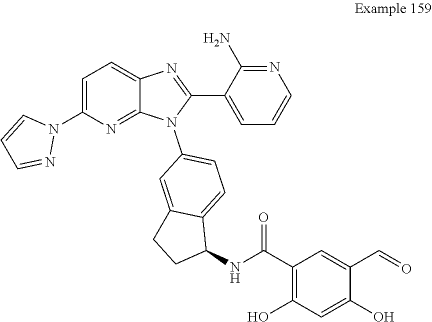 [SMILES: Nc1ncccc1-c1nc2ccc(-n3cccn3)nc2n1-c1ccc2c(c1)CC[C@@H]2NC(=O)c1cc(C=O)c(O)cc1O]

### Synthetic Route:

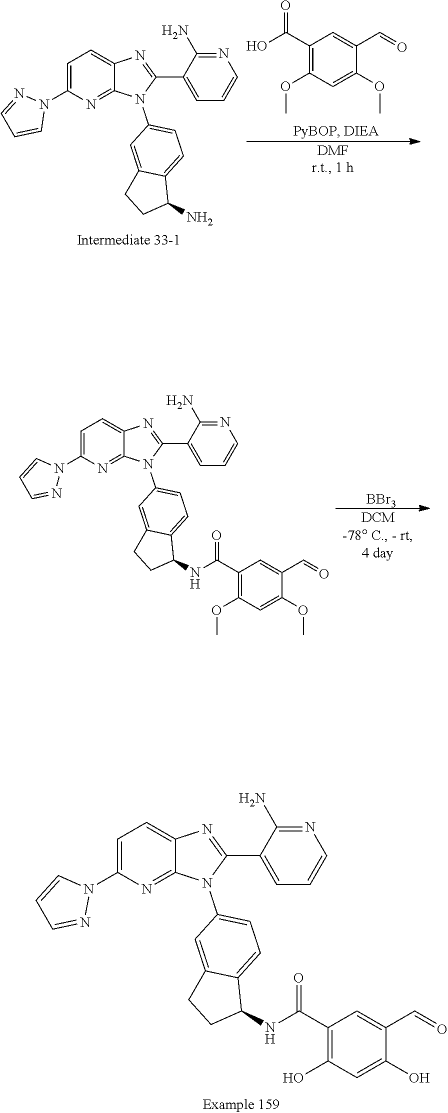 [SMILES: Nc1ncccc1-c1nc2ccc(-n3cccn3)nc2n1-c1ccc2c(c1)CC[C@@H]2N ; COc1cc(OC)c(C(=O)O)cc1C=O ; COc1cc(OC)c(C(=O)N[C@H]2CCc3cc(-n4c(-c5cccnc5N)nc5ccc(-n6cccn6)nc54)ccc32)cc1C=O ; "BBr3" ; Nc1ncccc1-c1nc2ccc(-n3cccn3)nc2n1-c1ccc2c(c1)CC[C@@H]2NC(=O)c1cc(C=O)c(O)cc1O]

### Step 1: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-2,4-dimethoxybenzamide

To a solution of (S)-3-(3-(1-amino-2,3-dihydro-1H-inden-5-yl)-5-(1H-pyrazol-1-yl)-3H-imidazo[4,5-b]pyridin-2-yl)pyridin-2-amine (Intermediate 33-1) (200 mg, 0.490 mmol, 1 equiv) in N,N-dimethylformamide (5 mL) was added N,N-diisopropylethylamine (632 mg, 4.90 mmol, 10 equiv), PyBOP (306 mg, 0.588 mmol, 1.2 equiv) and 5-formyl-2,4-dimethoxybenzoic acid (124 mg, 0.588 mmol, 1.2 equiv). The resulting mixture was stirred at room temperature for 10 min. The mixture was purified by reverse-phase flash column chromatography on C18 silica gel using a 20-70% gradient of acetonitrile in water (+0.05% ammonium bicarbonate) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-2,4-dimethoxybenzamide (200 mg, 67%) as a yellow solid. MS (ESI) calcd. for C₃₃H₂₈N₈O₄: 600.22 m/z, found 601.30 [M+H]⁺.

### Step 2: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-2,4-dihydroxybenzamide (Example 159)

To a cooled (−78° C.) solution of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-2,4-dimethoxybenzamide (100 mg, 0.166 mmol, 1 equiv) in dichloromethane (20 mL) was added 1M boron tribromide (1.66 mL, 1.66 mmol, 10 equiv) and the resulting mixture was stirred at room temperature for 4 days. The mixture were concentrated to dryness and the residue was purified by preparative HPLC on a XBridge Prep OBD Column using a 25-45% gradient of acetonitrile in water (+10 mmol/L ammonium bicarbonate) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-5-formyl-2,4-dihydroxybenzamide (Example 159) (16 mg, 16%) as an off-white solid. MS (ESI) calcd. for C₃₁H₂₄N₈O₄: 572.19 m/z, found 573.25 [M+H]⁺. ¹H NMR (400 MHz, DMSO-d₆) δ (ppm): 9.76 (s, 1H), 8.33-8.36 (m, 2H), 8.25 (s, 1H), 7.98-7.99 (m, 1H), 7.93-7.95 (m, 1H), 7.78-7.79 (m, 1H), 7.35-7.39 (m, 2H), 7.27-7.30 (m, 2H), 6.54-6.55 (m, 1H), 6.47-6.49 (m, 1H), 6.12-6.13 (m, 1H), 5.54-5.58 (m, 1H), 2.98-3.00 (m, 1H), 2.86-2.90 (m, 1H), 2.50-2.52 (m, 1H), 1.97-2.01 (m, 1H).

### Example 160: N-[(1S)-5-[2-(2-aminopyridin-3-yl)-5-{4H,5H,6H-pyrrolo[3,4-c]pyrazol-2-yl}imidazo[4,5-b]pyridin-3-yl]-2,3-dihydro-1H-inden-1-yl]-3-formyl-4-hydroxybenzamide

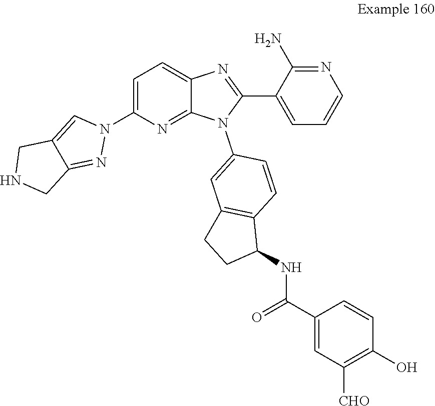 [SMILES: Nc1ncccc1-c1nc2ccc(-n3cc4c(n3)CNC4)nc2n1-c1ccc2c(c1)CC[C@@H]2NC(=O)c1ccc(O)c(C=O)c1]

Example 160 was prepared in a manner analogous to Example 94 using Intermediate 160-1 in place of Intermediate 94-1 and tert-butyl 2H,4H,6H-pyrrolo[3,4-c]pyrazole-5-carboxylate in place of 4-(difluoromethyl)-1H-pyrazole. MS (ESI) calcd. for C₃₃H₂₇N₉O₃: 597.22 m/z, found 598.50 [M+H]⁺. ¹H NMR (400 MHz, DMSO-d₆) δ (ppm): 10.28 (s, 1H), 8.21-8.42 (m, 3H), 7.95-8.14 (m, 2H), 7.79-7.95 (m, 1H), 7.42-7.68 (m, 2H), 7.19-7.38 (m, 3H), 7.18-6.92 (m, 2H), 6.38-6.59 (m, 1H), 5.36-5.79 (m, 1H), 4.05-4.44 (m, 4H), 2.72-3.15 (m, 2H), 2.39-2.51 (m, 1H), 1.95-2.19 (m, 1H).

### Intermediate 160-1: (S)—N-(5-(2-(2-aminopyridin-3-yl)-5-chloro-3H-imidazo[4,5-b]pyridin-3-yl)-2,3-dihydro-1H-inden-1-yl)-3-(1,3-dioxolan-2-yl)-4-((4-methoxybenzyl)oxy)benzamide

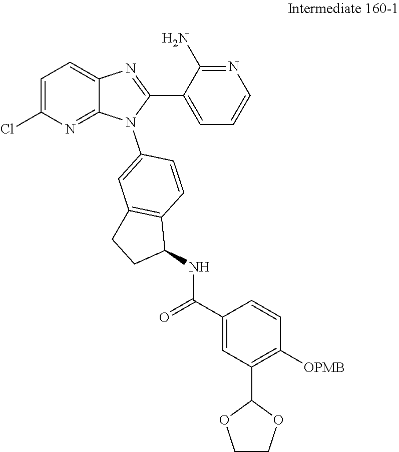

Intermediate 160-1 was prepared in a manner analogous to Intermediate 94-1 using PyBOP in place of HATU and Intermediate 5-1 in place of Intermediate 57-2. MS (ESI) calcd. for C₃₈H₃₃ClN₆O₅: 688.22 m/z, found 690.10 [M+H]⁺.

### Example 161: (S)-(4-((5-(2-(2-aminopyridin-3-yl)-5-phenyl-3H-imidazo[4,5-b]pyridin-3-yl)-2,3-dihydro-1H-inden-1-yl)carbamoyl)-2-formylphenyl)boronic acid

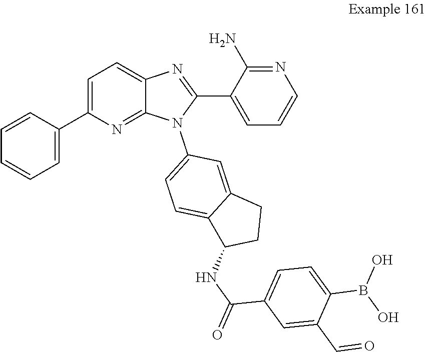 [SMILES: Nc1ncccc1-c1nc2ccc(-c3ccccc3)nc2n1-c1ccc2c(c1)CC[C@@H]2NC(=O)c1ccc(B(O)O)c(C=O)c1]

### Synthetic Route:

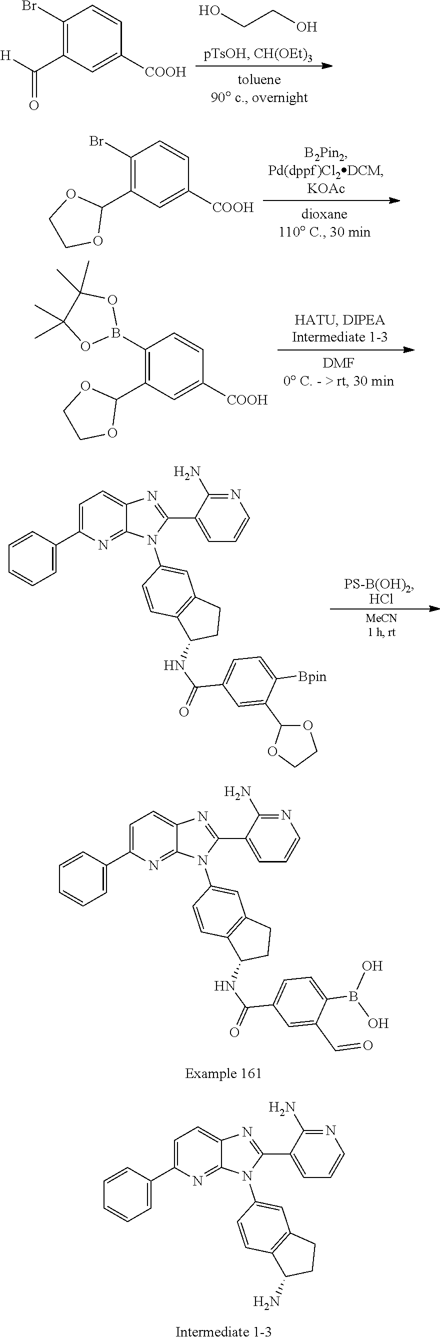

### Step 1: Synthesis of 4-bromo-3-(1,3-dioxolan-2-yl)benzoic acid

A suspension of 4-bromo-3-formylbenzoic acid (526 mg, 2.3 mmol, 1 equiv), ethylene glycol (998 mg, 16.1 mmol, 7 equiv), triethyl orthoformate (1.02 g, 6.89 mmol, 3 equiv) and p-toluenesulfonic acid (20 mg, 115 umol, 0.05 equiv) in toluene (5 mL) was stirred at room temperature for 10 min then at 90° C. overnight. The reaction mixture was concentrated under reduced pressure and the residue was purified by silica gel column chromatography using a 0-80% gradient of ethyl acetate in hexanes to afford 4-bromo-3-(1,3-dioxolan-2-yl)benzoic acid (91 mg, 15%) as a white solid. MS (ESI) calculated for C₁₀H₉BrO₄: 271.97 m/z, found 273.10 A suspension of 4-bromo-3-formylbenzoic acid (526 mg, 2.3 mmol, 1 equiv), ethylene glycol (998 mg, 16.1 mmol, 7 equiv), triethyl orthoformate (1.02 g, 6.89 mmol, 3 equiv) and p-toluenesulfonic acid (20 mg, 115 umol, 0.05 equiv) in toluene (5 mL) was stirred at room temperature for 10 min then at 90° C. overnight. The reaction mixture was concentrated under reduced pressure and the residue was purified by silica gel column chromatography using a 0-80% gradient of ethyl acetate in hexanes to afford 4-bromo-3-(1,3-dioxolan-2-yl)benzoic acid (91 mg, 15%) as a white solid. MS (ESI) calculated for C₁₀H₉BrO₄: 271.97 m/z, found 273.10.

### Step 2: Synthesis of 3-(1,3-dioxolan-2-yl)-4-(4,4,5,5-tetramethyl-1,3,2-dioxaborolan-2-yl)benzoic acid

A vial charged with 4-bromo-3-(1,3-dioxolan-2-yl)benzoic acid (91 mg, 333 umol, 1 equiv), 4,4,5,5-tetramethyl-2-(4,4,5,5-tetramethyl-1,3,2-dioxaborolan-2-yl)-1,3,2-dioxaborolane (85 mg, 333 μmol, 1 equiv), [1,1′-bis(diphenylphosphino)ferrocene]dichloropalladium(II)dichloromethane complex (27 mg, 33 umol, 0.1 equiv) and potassium acetate (164 mg, 1.67 mmol, 5 equiv) was purged and filled with nitrogen 3 times. 1,4-Dioxane (3 mL) was added and the mixture was sparged with nitrogen for 5 min. The resulting mixture was stirred at 110° C. for 30 min. The reaction was quenched with saturated aqueous ammonium chloride and brine. The mixture was extracted with ethyl acetate twice. The combined organic layers were dried over magnesium sulfate, filtered, and concentrated under reduced pressure. The resulting residue was purified by silica gel column chromatography using a 0-80% gradient of ethyl acetate in hexanes to afford 3-(1,3-dioxolan-2-yl)-4-(4,4,5,5-tetramethyl-1,3,2-dioxaborolan-2-yl)benzoic acid (75 mg, 70%) as a clear, colorless oil. MS (ESI) calculated for C₁₆HBO₆: 320.14 m/z, found 321.25.

### Step 3: Synthesis of (S)—N-(5-(2-(2-aminopyridin-3-yl)-5-phenyl-3H-imidazo[4,5-b]pyridin-3-yl)-2,3-dihydro-1H-inden-1-yl)-3-(1,3-dioxolan-2-yl)-4-(4,4,5,5-tetramethyl-1,3,2-dioxaborolan-2-yl)benzamide

To a cooled (0° C.) solution of 3-(1,3-dioxolan-2-yl)-4-(4,4,5,5-tetramethyl-1,3,2-dioxaborolan-2-yl)benzoic acid (73 mg, 229 umol, 1.2 equiv) in N,N-dimethylformamide (2 mL) was added HATU (109 mg, 287 umol, 1.5 equiv) and N,N-diisopropylethylamine (74 mg, 575 umol, 3 equiv) and the resulting mixture was stirred for 30 min at 0° C. A solution of (S)-3-(3-(1-amino-2,3-dihydro-1H-inden-5-yl)-5-phenyl-3H-imidazo[4,5-b]pyridin-2-yl)pyridin-2-amine (Intermediate 1-3) (80 mg, 191 umol, 1 equiv) and N,N-diisopropylethylamine (74 mg, 575 umol, 3 equiv) in N,N-dimethylformamide (2 mL) was added and the mixture was stirred for 1 h at room temperature. The reaction mixture was purified by preparative HPLC on a Phenomenex Gemini C18 column using a 10-100% gradient of acetonitrile in water (+0.05% 2,2,2-trifluoroacetic acid) to afford (S)—N-(5-(2-(2-aminopyridin-3-yl)-5-phenyl-3H-imidazo[4,5-b]pyridin-3-yl)-2,3-dihydro-1H-inden-1-yl)-3-(1,3-dioxolan-2-yl)-4-(4,4,5,5-tetramethyl-1,3,2-dioxaborolan-2-yl)benzamide as a pale-yellow solid (69 mg, 50%). MS (ESI) calculated for C₄₁H₄₂BN₆O₅: 720.32 m/z, found 721.30.

### Step 4: Synthesis of (S)-(4-((5-(2-(2-aminopyridin-3-yl)-5-phenyl-3H-imidazo[4,5-b]pyridin-3-yl)-2,3-dihydro-1H-inden-1-yl)carbamoyl)-2-formylphenyl)boronic acid (Example 161)

To a solution of (S)—N-(5-(2-(2-aminopyridin-3-yl)-5-phenyl-3H-imidazo[4,5-b]pyridin-3-yl)-2,3-dihydro-1H-inden-1-yl)-3-(1,3-dioxolan-2-yl)-4-(4,4,5,5-tetramethyl-1,3,2-dioxaborolan-2-yl)benzamide (60 mg, 83 umol, 1 equiv) in acetonitrile (5 mL) was added polymer bound boronic acid (120 mg, Sigma 632627) and hydrochloric acid (1M, 5 mL). The resulting mixture was stirred for 1 h at room temperature. The mixture was filtered, rinsing with methanol. The filtrate was concentrated to dryness under reduced pressure. The residue was purified by preparative HPLC on a Phenomenex Gemini C18 column using a 10-100% gradient of acetonitrile in water (+0.05% 2,2,2-trifluoroacetic acid) to afford (S)-(4-((5-(2-(2-aminopyridin-3-yl)-5-phenyl-3H-imidazo[4,5-b]pyridin-3-yl)-2,3-dihydro-1H-inden-1-yl)carbamoyl)-2-formylphenyl)boronic acid (Example 161) as a pale-yellow solid (37 mg, 75%). MS (ESI) calculated for C₃₄H₂₇BN₆O₄: 594.22 m/z, found 595.30. ¹H NMR (300 MHz, DMSO-d₆) δ (ppm): 10.11 (s, 1H), 9.09 (d, J=8.3 Hz, 1H), 8.37 (d, J=1.8 Hz, 1H), 8.24 (d, J=8.3 Hz, 1H), 8.09 (dd, J=7.7, 1.8 Hz, 1H), 8.01-7.92 (m, 4H), 7.62 (d, J=7.7 Hz, 1H), 7.50 (s, 1H), 7.46-7.26 (m, 6H), 6.60 (s, 1H), 5.62 (q, J=8.1 Hz, 1H), 3.03-2.93 (m, 1H), 2.91-2.82 (m, 1H), 2.13-2.02 (m, 1H). (2,2,2-trifluoroacetic acid salt).

### Example 162: N-[(1S)-5-[2-(2-aminopyridin-3-yl)-5-phenylimidazo[4,5-b]pyridin-3-yl]-2,3-dihydro-1H-inden-1-yl]-7-formyl-1H-1,3-benzodiazole-5-carboxamide

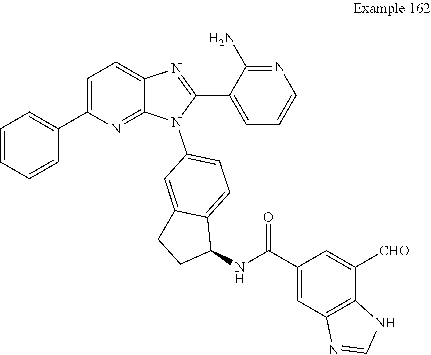 [SMILES: Nc1ncccc1-c1nc2ccc(-c3ccccc3)nc2n1-c1ccc2c(c1)CC[C@@H]2NC(=O)c1cc(C=O)c2[nH]cnc2c1]

Example 162 was prepared in a manner analogous to Example 7 using Intermediate 1-3 in place of Intermediate 1-2 and Intermediate 162-1 in place of Intermediate 5-1. MS (ESI) calcd. for C₃₅H₂₆N₈O₂: 590.22 m/z, found 591.25 [M+H]⁺. ¹H NMR (400 MHz, DMSO-d₆) δ (ppm): 10.24 (s, 1H), 8.55-8.69 (m, 1H), 8.40-8.55 (m, 2H), 8.10-8.40 (m, 1H), 7.87-8.10 (m, 4H), 7.27-7.55 (m, 7H), 6.45-6.70 (m, 1H), 5.55-5.82 (m, 1H), 3.01-3.25 (m, 1H), 2.81-3.01 (m, 1H), 2.61-2.72 (m, 1H), 2.00-2.28 (m, 1H).

### Intermediate 162-1: 7-(1,3-dioxolan-2-yl)-1-((2-(trimethylsilyl)ethoxy)methyl)-1H-benzo[d]imidazole-5-carboxylic acid

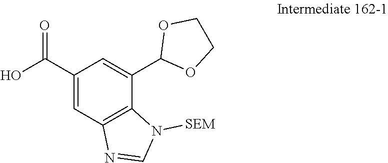 [SMILES: C[Si](C)(C)CCOCn1cnc2cc(C(=O)O)cc(C3OCCO3)c21]

### Synthetic Route:

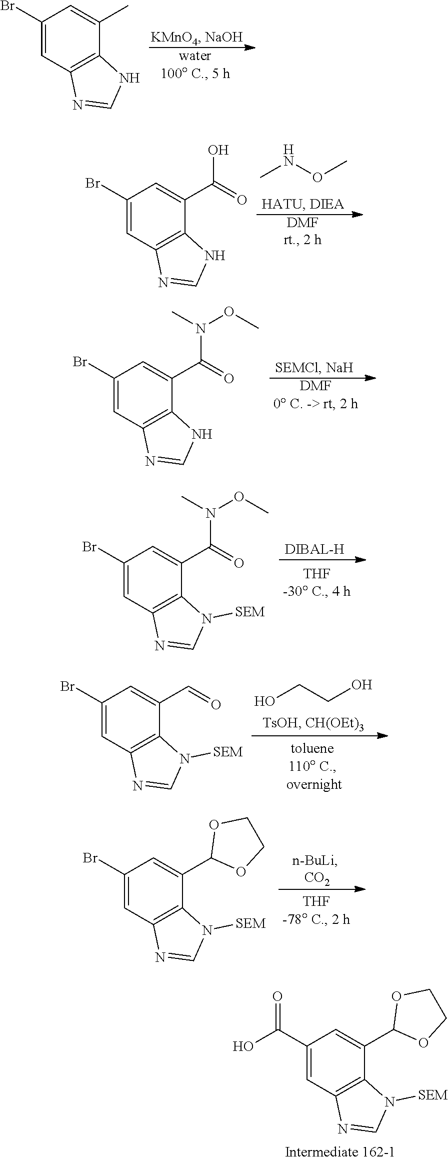

### Step 1: Synthesis of 6-bromo-3H-1,3-benzodiazole-4-carboxylic acid

A solution of 6-bromo-4-methyl-3H-1,3-benzodiazole (5.00 g, 23.7 mmol, 1 equiv) and potassium permanganate (5.62 g, 35.5 mmol, 1.5 equiv) in 0.5 M sodium hydroxide (240 mL) was stirred at 100° C. for 10 h. The resulting mixture was filtered, rinsing with water (100 mL). The filtrate was concentrated under reduced pressure and the mixture was acidified to pH 5 with 4N aq. hydrochloric acid. The precipitated solids were collected by filtration, washed with water and dried under vacuum to afford 6-bromo-3H-1,3-benzodiazole-4-carboxylic acid (3.5 g, 61%) as a light grey solid, which was used in the next step directly without further purification. MS (ESI) calcd. for C₈H₅BrN₂O₂: 239.95 m/z, found 240.95 [M+H]⁺.

### Step 2: Synthesis of 6-bromo-N-methoxy-N-methyl-3H-1,3-benzodiazole-4-carboxamide

A solution of 6-bromo-3H-1,3-benzodiazole-4-carboxylic acid (3.5 g, 14.5 mmol, 1 equiv), N,O-dimethylhydroxylamine (1.77 g, 29 mmol, 2 equiv), HATU (8.28 g, 21.8 mmol) and N,N-diisopropylethylamine (7.51 g, 58.1 mmol) in N,N-dimethylformamide (40 mL) was stirred for 0.5 h at 25° C. The mixture was purified by the reverse phase flash column chromatography on C18 silica gel using a 29-60% gradient of acetonitrile in water (+0.05% ammonium bicarbonate) to afford 6-bromo-N-methoxy-N-methyl-3H-1,3-benzodiazole-4-carboxamide (1.6 g, 38%) as a yellow solid. MS (ESI) calcd. for C₁₀H₁₀BrN₃O₂: 283.00 m/z, found 284.00 [M+H]⁺.

### Step 3: Synthesis of 6-bromo-N-methoxy-N-methyl-3-{[2-(trimethylsilyl)ethoxy]methyl}-1,3-benzodiazole-4-carboxamide

To a cooled (0° C.) solution of 6-bromo-N-methoxy-N-methyl-3H-1,3-benzodiazole-4-carboxamide (1.4 g, 4.9 mmol, 1 equiv) in tetrahydrofuran (49 mL) was added 60% sodium hydride (0.18 g, 7.4 mmol, 1.5 equiv) and the obtained suspension was stirred temperature for 30 min. (2-(Chloromethoxy)ethyl)trimethylsilane (1.64 g, 9.86 mmol, 2 equiv) was added and the mixture was stirred at room temperature for 2 h. the reaction was quenched with water and extracted with dichloromethane (2×100 mL). The combined organic layers were washed with saturated aqueous ammonium chloride, dried over anhydrous sodium sulfate, filtered and concentrated under reduced pressure. The crude material was purified by reverse-phase column chromatography on C18 silica gel using a 15-70% gradient of acetonitrile in water (+0.05% ammonium bicarbonate) to afford 6-bromo-N-methoxy-N-methyl-3-{[2-(trimethylsilyl)ethoxy]methyl}-1,3-benzodiazole-4-carboxamide (700 mg, 33%) as a colorless oil. MS (ESI) calcd. for C₁₆H₂₄BrN₃O₃Si: 413.08 m/z, found 414.08 [M+H]⁺.

### Step 4: Synthesis of 6-bromo-3-{[2-(trimethylsilyl)ethoxy]methyl}-1,3-benzodiazole-4-carbaldehyde

To a cooled (−78° C.) solution of 6-bromo-N-methoxy-N-methyl-3-{[2-(trimethylsilyl)ethoxy]methyl}-1,3-benzodiazole-4-carboxamide (400 mg, 0.965 mmol, 1 equiv) in tetrahydrofuran (14 mL) was added 1M diisobutylaluminium hydride (2.9 mL, 2.9 mmol, 3 equiv) and the resulting mixture was stirred at −78° C. for 3 h. The reaction was quenched with saturated aqueous potassium sodium tartrate and the mixture was extracted with ethyl acetate (3×50 mL). The combined organic layers were washed with brine, dried over anhydrous sodium sulfate, filtered and concentrated under reduced pressure. The crude was purified by silica gel column chromatography using a 0-100% gradient of ethyl acetate in petroleum ether to afford 6-bromo-3-{[2-(trimethylsilyl)ethoxy]methyl}-1,3-benzodiazole-4-carbaldehyde (420 mg, 73%) as a colorless oil. MS (ESI) calcd. for C₁₄H₁₉BrN₂O₂Si: 354.04 m/z, found 355.00 [M+H]⁺.

### Step 5: Synthesis of 5-bromo-7-(1,3-dioxolan-2-yl)-1-{[2-(trimethylsilyl)ethoxy]methyl}-1,3-benzodiazole

A solution of 6-bromo-3-{[2-(trimethylsilyl)ethoxy]methyl}-1,3-benzodiazole-4-carbaldehyde (420 mg, 1.18 mmol, 1 equiv), ethylene glycol (367 mg, 5.91 mmol, 5 equiv), p-toluenesulfonic acid (41 mg, 0.236 mmol, 0.2 equiv), and triethyl orthoformate (526 mg, 3.55 mmol, 3 equiv) in toluene (20 mL) was stirred at 110° C. for 4 h. The mixture was cooled to room temperature and water was added. The resulting mixture was extracted with ethyl acetate (3×50 mL). The combined organic layers were washed with brine, dried over anhydrous sodium sulfate, filtered, and concentrated under reduced pressure. The crude was purified by silica gel column chromatography using a 0-100% gradient of ethyl acetate in petroleum ether to provide 5-bromo-7-(1,3-dioxolan-2-yl)-1-{[2-(trimethylsilyl)ethoxy]methyl}-1,3-benzodiazole (230 mg, 48%) as colorless oil. MS (ESI) calcd. for C₁₆H₂₃BrN₂O₃Si: 398.07 m/z, found 401.05 [M+H+2]⁺.

### Step 6: Synthesis of 7-(1,3-dioxolan-2-yl)-1-{[2-(trimethylsilyl)ethoxy]methyl}-1,3-benzodiazole-5-carboxylic acid (Intermediate 162-1)

To a cooled (−78° C.) solution of 5-bromo-7-(1,3-dioxolan-2-yl)-1-{[2-(trimethylsilyl)ethoxy]methyl}-1,3-benzodiazole (200 mg, 0.501 mmol, 1 equiv) in tetrahydrofuran (10 mL) was added n-butyllithium (2.5M, 0.30 mL, 0.75 mmol, 1.5 equiv) and the resulting mixture was stirred at −78° C. for 1 h under nitrogen atmosphere then for a further 1 h under carbon dioxide. The reaction was warmed to 0° C. and quenched with water (1 mL). The resulting mixture was concentrated under reduced pressure and the crude was purified by reverse-phase flash column chromatography on C18 silica gel using a 20-65% gradient of acetonitrile in water (+0.1% formic acid) to afford 7-(1,3-dioxolan-2-yl)-1-{[2-(trimethylsilyl)ethoxy]methyl}-1,3-benzodiazole-5-carboxylic acid (Intermediate 162-1) (80 mg, 38%) as a white solid. MS (ESI) calcd. for C₁₇H₂₄N₂O₅Si: 364.15 m/z, found 365.15 [M+H]⁺.

### Example 163: (S)—N-(5-(2-(2-amino-5-fluoropyridin-3-yl)-5-(1H-pyrazol-1-yl)-3H-imidazo[4,5-b]pyridin-3-yl)-2,3-dihydro-1H-inden-1-yl)-3-formyl-4-hydroxybenzamide

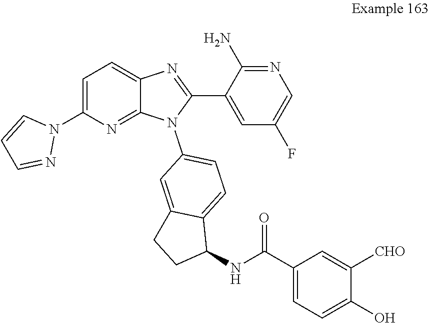 [SMILES: Nc1ncc(F)cc1-c1nc2ccc(-n3cccn3)nc2n1-c1ccc2c(c1)CC[C@@H]2NC(=O)c1ccc(O)c(C=O)c1]

Example 163 was prepared in a manner analogous to Example 33 (via Intermediate 33-1) starting from Intermediate 163-2 in place of Intermediate 18-2, using a reaction time of 2 h instead of overnight for the EPhos coupling, PyBOP in place of HATU and Intermediate 57-2 in place of Intermediate 5-1. MS (ESI) calcd. for C₃₁H₂₃FN₈O₃: 574.19 m/z, found 575.20 [M+H]⁺. ¹H NMR (400 MHz, DMSO-d₆) δ (ppm): 10.27 (s, 1H), 8.35-8.38 (m, 2H), 8.28-8.29 (m, 1H), 8.06-8.08 (m, 2H), 7.95-7.98 (m, 1H), 7.80-7.81 (m, 1H), 7.31-7.41 (m, 4H), 7.06-7.08 (m, 1H), 6.54-6.55 (m, 1H), 5.58-5.62 (m, 1H), 3.01-3.07 (m, 1H), 2.88-2.94 (m, 1H), 2.53-2.54 (m, 1H), 2.08-2.11 (m, 1H). ¹⁹F NMR (376 MHz, DMSO-d₆) δ (ppm): −143.74. (2,2,2-trifluoroacetic acid salt).

### Intermediate 163-2: (S)—N-(5-(2-(2-amino-5-fluoropyridin-3-yl)-5-chloro-3H-imidazo[4,5-b]pyridin-3-yl)-2,3-dihydro-1H-inden-1-yl)acetamide

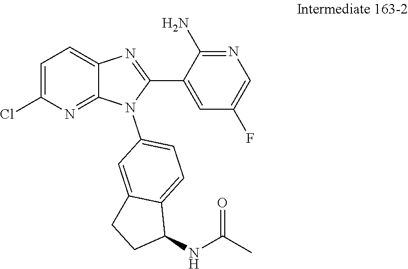 [SMILES: CC(=O)N[C@H]1CCc2cc(-n3c(-c4cc(F)cnc4N)nc4ccc(Cl)nc43)ccc21]

### Synthetic Route:

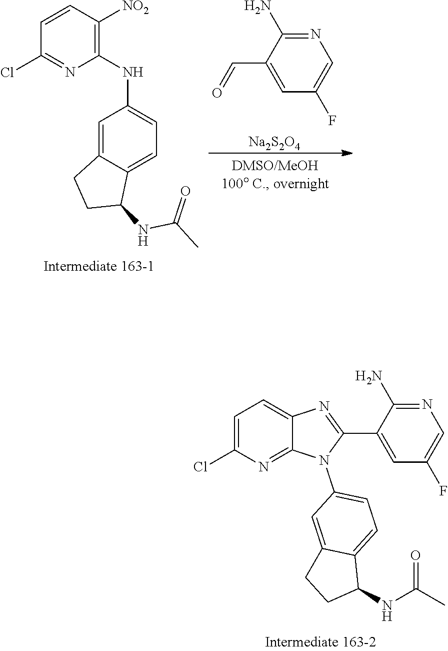

### Step 1: Synthesis of (S)—N-(5-(2-(2-amino-5-fluoropyridin-3-yl)-5-chloro-3H-imidazo[4,5-b]pyridin-3-yl)-2,3-dihydro-1H-inden-1-yl)acetamide (Intermediate 163-2)

A mixture of (S)—N-(5-((6-chloro-3-nitropyridin-2-yl)amino)-2,3-dihydro-1H-inden-1-yl)acetamide (Intermediate 163-1) (3.09 g, 8.92 mmol, 1 equiv), 2-amino-5-fluoronicotinaldehyde (1.50 g, 10.7 mmol, 1.2 equiv) and sodium dithionite (3.88 g, 22.3 mmol, 2.5 equiv) in dimethyl sulfoxide (12 mL) and methanol (2 mL) was stirred at 100° C. overnight. The mixture was cooled to room temperature and purified by reverse-phase flash column chromatography on C18 silica gel using a 10-70% gradient of acetonitrile in water (+0.05% ammonium bicarbonate) to afford (S)—N-(5-(2-(2-amino-5-fluoropyridin-3-yl)-5-chloro-3H-imidazo[4,5-b]pyridin-3-yl)-2,3-dihydro-1H-inden-1-yl)acetamide (Intermediate 163-2) (500 mg, 13%) as a yellow solid. MS (ESI) calcd. for C₂₂H₁₈ClFN₆O: 436.12 m/z, found 437.20 [M+H]⁺.

### Intermediate 163-1: (S)—N-(5-((6-chloro-3-nitropyridin-2-yl)amino)-2,3-dihydro-1H-inden-1-yl)acetamide

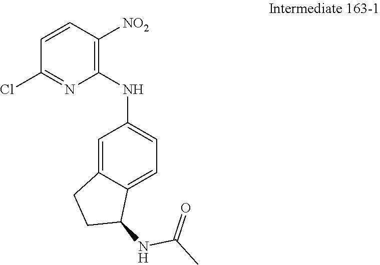 [SMILES: CC(=O)N[C@H]1CCc2cc(Nc3nc(Cl)ccc3[N+](=O)[O-])ccc21]

### Synthetic Route:

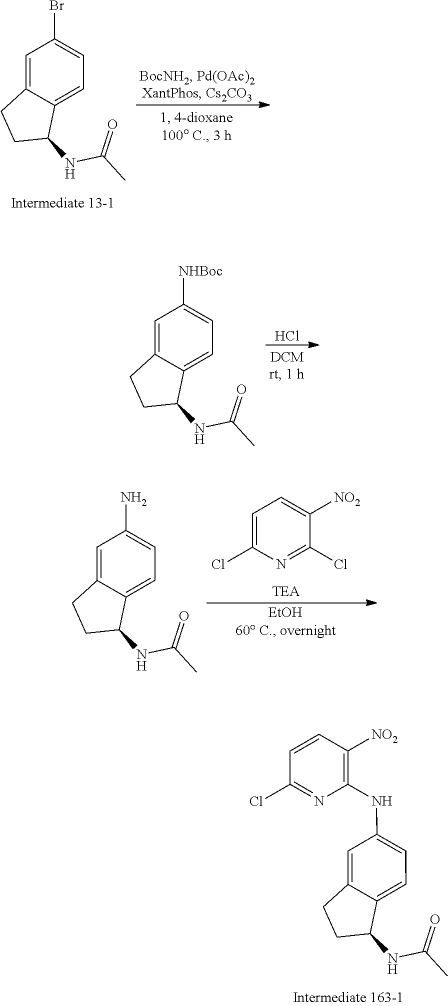 [SMILES: CC(=O)N[C@H]1CCc2cc(Br)ccc21 ; CC(=O)N[C@H]1CCc2cc(NC(=O)OC(C)(C)C)ccc21 ; Cl ; CC(=O)N[C@H]1CCc2cc(N)ccc21 ; O=[N+]([O-])c1ccc(Cl)nc1Cl ; CC(=O)N[C@H]1CCc2cc(Nc3nc(Cl)ccc3[N+](=O)[O-])ccc21]

### Step 1: Synthesis of tert-butyl (S)-(1-acetamido-2,3-dihydro-1H-inden-5-yl)carbamate

To a mixture of N-[(1S)-5-bromo-2,3-dihydro-1H-inden-1-yl]acetamide (Intermediate 13-1) (40 g, 157 mmol, 1 equiv), tert-butyl carbamate (27.66 g, 236 mmol, 1.5 equiv), XantPhos (9.11 g, 15.7 mmol, 10 mol %), palladium (II) acetate (3.54 g, 15.7 mmol, 10 mol %), and cesium carbonate (154 g, 472 mmol, 10 mol %) was added 1,4-dioxane (300 mL) under nitrogen atmosphere. The resulting mixture was stirred for 3 h at 100° C. The reaction mixture was quenched by addition of water (50 mL) and extracted with ethyl acetate (3×50 mL). The combined organic layers were dried over anhydrous sodium sulfate and concentrated under reduced pressure. The residue was purified by silica gel column chromatography using an eluent of petroleum ether/dichloromethane/methanol (70:27:3) to afford tert-butyl N-[(1S)-1-acetamido-2,3-dihydro-1H-inden-5-yl]carbamate (43.1 g, 48%). MS (ESI) calculated for C₁₆H₂₂N₂O₃: 290.16 m/z, found 289.05 [M−H]⁻.

### Step 2: Synthesis of (S)—N-(5-amino-2,3-dihydro-1H-inden-1-yl)acetamide

To a stirred solution of tert-butyl N-[(1S)-1-acetamido-2,3-dihydro-1H-inden-5-yl]carbamate (43.1 g, 148 mmol, 1 equiv) in dichloromethane (180 mL) was added 4N hydrochloric acid in 1,4-dioxane (185 mL, 742 mmol, 5 equiv). The reaction mixture was stirred for 1 h at room temperature. The reaction mixture was concentrated in vacuo and re-crystallized from ethyl acetate to afford N-[(1S)-5-amino-2,3-dihydro-1H-inden-1-yl]acetamide (hydrochloride salt) (23 g, 81%) as a white solid. MS (ESI) calculated for C₁₁H₁₄N₂O: 190.11 m/z, found 191.15 [M+H]⁺.

### Step 3: Synthesis of N-[(1S)-5-[(6-chloro-3-nitropyridin-2-yl)amino]-2,3-dihydro-1H-inden-1-yl]acetamide (Intermediate 163-1)

To a solution of N-[(1S)-5-amino-2,3-dihydro-1H-inden-1-yl]acetamide (10.0 g, 52.6 mmol, 1 equiv) in ethanol (200 mL) was added triethylamine (16.0 g, 0.158 mol, 3 equiv) and 2,6-dichloro-3-nitropyridine (12.2 g, 63.1 mmol, 1.2 equiv). The resulting mixture was stirred at 60° C. overnight. The mixture was then cooled to room temperature and quenched with water. The resulting precipitate was collected by filtration and rinsed with ethanol/water to afford N-[(1S)-5-[(6-chloro-3-nitropyridin-2-yl)amino]-2,3-dihydro-1H-inden-1-yl]acetamide (Intermediate 163-1) (100 g, 49%) as a red solid. MS (ESI) calculated for C₁₆H₁₅ClN₄O₃: 346.08 m/z, found 345.00 [M−H]⁻.

### Example 165: (S)—N-(5-(2-(2-aminopyridin-3-yl)-5-(1-(1-methylazetidin-3-yl)-1H-pyrazol-3-yl)-3H-imidazo[4,5-b]pyridin-3-yl)-2,3-dihydro-1H-inden-1-yl)-3-formyl-4-hydroxybenzamide

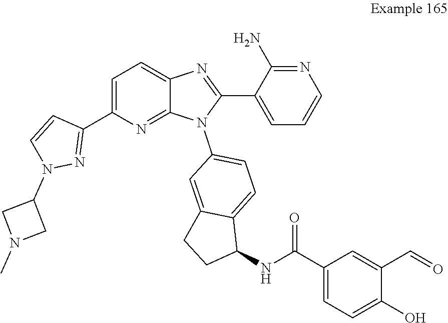 [SMILES: CN1CC(n2ccc(-c3ccc4nc(-c5cccnc5N)n(-c5ccc6c(c5)CC[C@@H]6NC(=O)c5ccc(O)c(C=O)c5)c4n3)n2)C1]

### Synthetic Route:

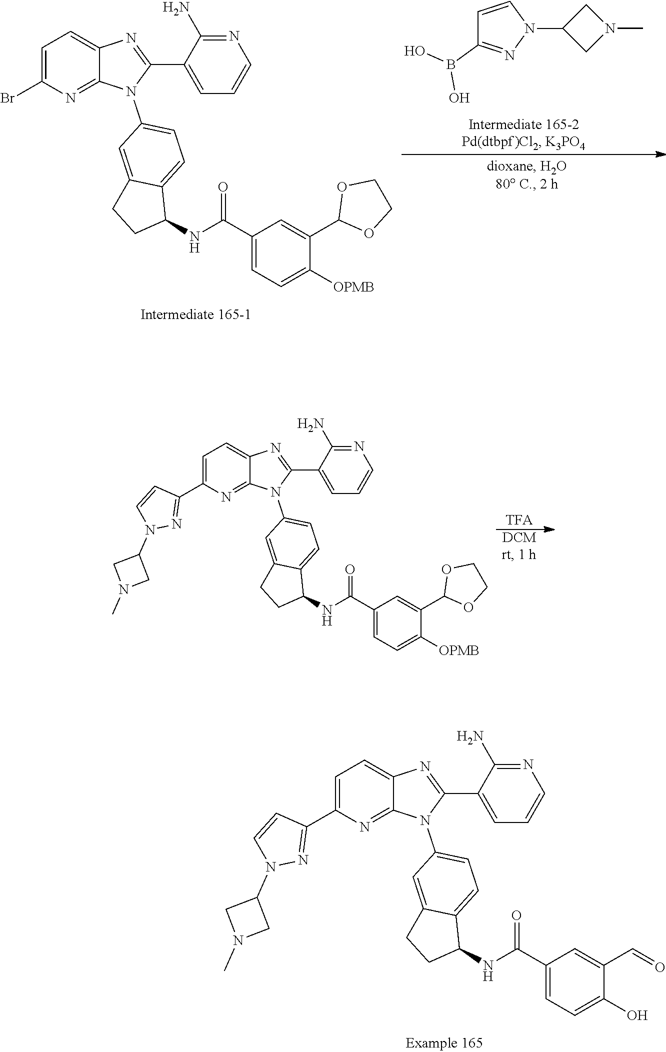

### Step 1: (S)—N-(5-(2-(2-aminopyridin-3-yl)-5-(1-(1-methylazetidin-3-yl)-1H-pyrazol-3-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide

To a solution of 1-(1-methylazetidin-3-yl)pyrazol-3-ylboronic acid (Intermediate 165-2) (200 mg, 1.11 mmol, 10 equiv), N-[(1S)-5-[2-(2-aminopyridin-3-yl)-5-bromoimidazo[4,5-b]pyridin-3-yl]-2,3-dihydro-1H-inden-1-yl]-3-(1,3-dioxolan-2-yl)-4-[(4-methoxyphenyl)methoxy]benzamide (Intermediate 165-1) (81 mg, 0.11 mmol, 1 equiv) and bis(di-tert-butylphosphino)ferrocene]dichloropalladium(II) (7.2 mg, 0.011 mmol, 0.1 equiv) in 1,4-dioxane (10 mL) was added a solution of tribasic potassium phosphate (59 mg, 0.28 mmol, 2.5 equiv) in water (2.5 mL). The resulting mixture was stirred for 2 h at 90° C. under nitrogen atmosphere. After cooling to room temperature, the reaction was quenched with water (10 mL) and the mixture was extracted with ethyl acetate (2×30 mL). The combined organic extracts were washed with brine (10 mL), dried over anhydrous sodium sulfate, filtered and concentrated to afford crude (S)—N-(5-(2-(2-aminopyridin-3-yl)-5-(1-(1-methylazetidin-3-yl)-1H-pyrazol-3-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide (100 mg, 11% yield) as a yellow solid, which was used without further purification in the next step. MS (ESI) calcd. for C₄₅H₄₃N₉O₅: 789.34 m/z, found 790.35 [M+H]⁺.

### Step 2: (S)—N-(5-(2-(2-aminopyridin-3-yl)-5-(1-(1-methylazetidin-3-yl)-1H-pyrazol-3-yl)-3H-imidazo[4,5-b]pyridin-3-yl)-2,3-dihydro-1H-inden-1-yl)-3-formyl-4-hydroxybenzamide (Example 165)

To a solution of N-[(1S)-5-[2-(2-aminopyridin-3-yl)-5-[1-(1-methylazetidin-3-yl)pyrazo]-3-yl]imidazo[4,5-b]pyridin-3-yl]-2,3-dihydro-1H-inden-1-yl]-3-(1,3-dioxolan-2-yl)-4-[(4-methoxyphenyl)methoxy]benzamide (100 mg, 0.127 mmol) in dichloromethane (2 mL) was added 2,2,2-trifluoroacetic acid (0.20 mL) and the resulting mixture was stirred at room temperature for 1 h. The solution was concentrated and the crude product was purified by preparative HPLC on a XBridge Prep OBD C18 Column using a 27-50% gradient of acetonitrile in water (+10 mmol/L ammonium bicarbonate) to afford N-[(1S)-5-[2-(2-aminopyridin-3-yl)-5-[1-(1-methylazetidin-3-yl)pyrazo]-3-yl]imidazo[4,5-b]pyridin-3-yl]-2,3-dihydro-1H-inden-1-yl]-3-formyl-4-hydroxybenzamide (Example 165) (6.5 mg, 8% yield) as a yellow solid. MS (ESI) calcd. for C₃₅H₃₁N₉O₃: 625.25 m/z, found 648.25 [M+Na]⁺. ¹H NMR (400 MHz, DMSO-d₆) δ 10.20-10.21 (m, 1H), 8.18-8.20 (m, 2H), 7.88-8.00 (m, 4H), 7.21-7.32 (m, 4H), 6.88-6.90 (m, 1H), 6.66-6.67 (m, 1H), 6.44-6.47 (m, 1H), 5.57-5.58 (m, 1H), 4.96-5.00 (m, 1H), 3.74-3.76 (m, 2H), 3.45-3.46 (m, 2H), 3.00-3.01 (m, 1H), 2.97-2.99 (m, 1H), 2.50-2.51 (m, 1H), 2.35-2.36 (m, 3H), 2.03-2.09 (m, 1H).

### Intermediate 165-1: (S)—N-(5-(2-(2-aminopyridin-3-yl)-5-bromo-3H-imidazo[4,5-b]pyridin-3-yl)-2,3-dihydro-1H-inden-1-yl)-3-(1,3-dioxolan-2-yl)-4-((4-methoxybenzyl)oxy)benzamide

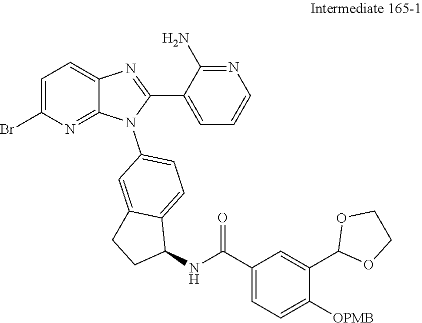

Intermediate 165-1 was prepared in a manner analogous to Intermediate 94-1 using Intermediate 18-2 in place of Intermediate 79-1, PyBOP in place of HATU and Intermediate 5-1 in place of Intermediate 57-2. MS (ESI) calcd. for C₃₈H₃₃BrN₆O₅: 732.17 m/z, found 733.20 [M+H]⁺.

### Intermediate 165-2: (1-(1-methylazetidin-3-yl)-1H-pyrazol-3-yl)boronic acid

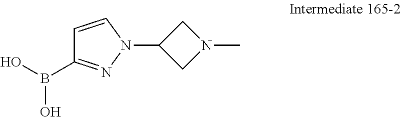

### Synthetic Route:

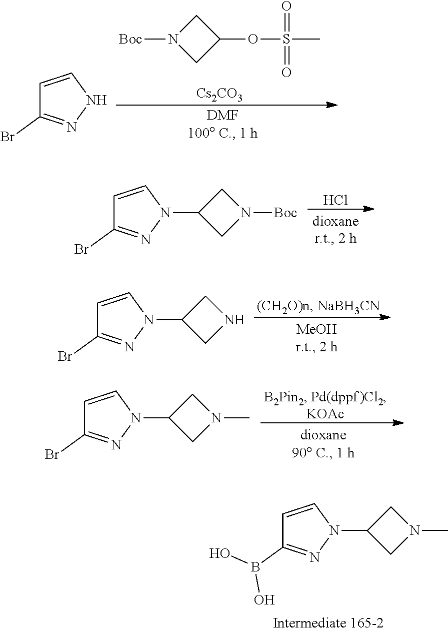

### Step 1: Synthesis of tert-butyl 3-(3-bromo-1H-pyrazol-1-yl)azetidine-1-carboxylate

To a solution of 3-bromo-1H-pyrazole (1.00 g, 6.80 mmol, 1 equiv) in N,N-dimethylformamide (8 mL) were added cesium carbonate (4.46 g, 13.7 mmol, 2 equiv) and tert-butyl 3-((methylsulfonyl)oxy)azetidine-1-carboxylate (2.56 g, 10.2 mmol, 1.5 equiv) and the obtained solution was stirred at room temperature for 2 h. The mixture was purified by reverse-phase flash column chromatography on C18 silica gel using a 10-60% gradient of acetonitrile in water (+0.05% ammonium bicarbonate) to afford tert-butyl 3-(3-bromopyrazol-1-yl)azetidine-1-carboxylate as a colorless oil (1.3 g, 63% yield). MS (ESI) calcd. for C₁₁H₁₆BrN₃O₂: 301.04 m/z, found 302.20 [M+H]⁺.

### Step 2: Synthesis of 1-(azetidin-3-yl)-3-bromo-1H-pyrazole

tert-Butyl 3-(3-bromopyrazol-1-yl)azetidine-1-carboxylate (1.3 g, 4.3 mmol, 1 equiv) was dissolved in 4N hydrochloric acid in 1,4-dioxane (15 mL). The mixture was stirred at room temperature for 1 h and the reaction mixture was concentrated to dryness to afford 1-(azetidin-3-yl)-3-bromo-1H-pyrazole as a yellow solid (1.2 g, crude quant), which was used directly in the next step without purification. MS (ESI) calcd. for C₆H₈BrN₃: 200.99 m/z, found 202.00 [M+H]⁺.

### Step 3: Synthesis of 3-bromo-1-(1-methylazetidin-3-yl)-1H-pyrazole

To a solution of 1-(azetidin-3-yl)-3-bromopyrazole (120 mg, 0.299 mmol, 1 equiv) and paraformaldehyde (560 mg, 18.6 mmol, 62 equiv) in methanol (12 mL) was added sodium cyanoborohydride (560 mg, 8.91 mmol, 30 equiv) and the resulting mixture was stirred at room temperature for 1 h. The resulting mixture was purified by reverse-phase flash column chromatography on C18 silica gel using a 5-60% gradient of acetonitrile in water (+0.05% ammonium bicarbonate) to afford 3-bromo-1-(1-methylazetidin-3-yl)pyrazole (350 mg, 27%) as a colorless oil. MS (ESI) calcd. for C₇H₁₀BrN₃: 215.01 m/z, found 216.00 [M+H]⁺.

### Step 4: Synthesis of (1-(1-methylazetidin-3-yl)-1H-pyrazol-3-yl)boronic acid (Intermediate 165-2)

To a mixture of 3-bromo-1-(1-methylazetidin-3-yl)pyrazole (300 mg, 1.39 mmol, 1 equiv), palladium (II) acetate (19 mg, 0.083 mmol, 0.06 equiv), potassium acetate (341 mg, 3.47 mmol, 2.5 equiv) and bis(pinacolato)diboron (423 mg, 1.67 mmol, 1.2 equiv) in 1,4-dioxane (5 mL) was added tricyclohexylphosphine (47 mg, 0.17 mmol, 0.12 mmol) and the resulting solution was stirred for 2 h at 100° C. under nitrogen atmosphere. The reaction mixture was concentrated to afford (1-(1-methylazetidin-3-yl)-1H-pyrazol-3-yl)boronic acid (Intermediate 165-2) as a light-yellow solid, which was used in subsequent transformations directly without further purification. MS (ESI) calcd. for C₇H₁₂BN₃O₂: 181.10 m/z, found 182.10 [M+H]⁺.

### Example 166: (S)—N-(5-(2-(2-amino-6-methylpyridin-3-yl)-5-(1H-pyrazol-1-yl)-3H-imidazo[4,5-b]pyridin-3-yl)-2,3-dihydro-1H-inden-1-yl)-3-formyl-4-hydroxybenzamide

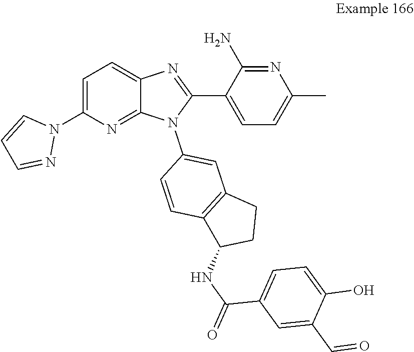 [SMILES: Cc1ccc(-c2nc3ccc(-n4cccn4)nc3n2-c2ccc3c(c2)CC[C@@H]3NC(=O)c2ccc(O)c(C=O)c2)c(N)n1]

Example 166 was prepared in a manner analogous to Example 33 (via Intermediate 33-1) starting from Intermediate 166-1 in place of Intermediate 18-2, using a reaction time of 2 h instead of overnight for the EPhos coupling, PyBOP in place of HATU and Intermediate 57-2 in place of Intermediate 5-1. MS (ESI) calcd. for C₃₂H₂₆N₈O₃: 570.21 m/z, found 571.20 [M+H]⁺. ¹H NMR (400 MHz, DMSO-d₆) δ (ppm): 10.30-10.31 (m, 1H), 8.89-8.91 (m, 1H), 8.33-8.35 (m, 2H), 8.29-8.30 (m, 1H), 8.06-8.08 (m, 1H), 7.92-7.95 (m, 1H), 7.79-7.80 (m, 1H), 7.40-7.41 (m, 1H), 7.35-7.37 (m, 1H), 7.26-7.28 (m, 1H), 7.11-7.13 (m, 1H), 7.00-7.02 (m, 3H), 6.54-6.55 (m, 1H), 6.30-6.32 (m, 1H), 5.64-5.66 (m, 1H), 3.00-3.10 (m, 1H), 2.93-2.94 (m, 1H), 2.50-2.51 (m, 1H), 2.26-2.31 (m, 3H), 2.10-2.15 (m, 1H).

### Intermediate 166-1: (S)—N-(5-(2-(2-amino-6-methylpyridin-3-yl)-5-bromo-3H-imidazo[4,5-b]pyridin-3-yl)-2,3-dihydro-1H-inden-1-yl)acetamide

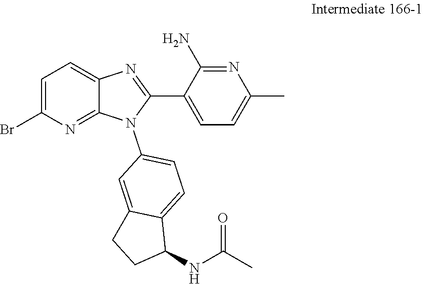 [SMILES: CC(=O)N[C@H]1CCc2cc(-n3c(-c4ccc(C)nc4N)nc4ccc(Br)nc43)ccc21]

Intermediate 166-1 was prepared in a manner analogous to Intermediate 163-2 using Intermediate 18-1 in place of Intermediate 163-1 and 2-amino-6-methylnicotinaldehyde in place of 2-amino-5-fluoronicotinaldehyde. MS (ESI) calcd. for C₂₃H₂₁BrN₆O: 476.10 m/z, found 477.20 [M+H]⁺.

### Example 167: (S)—N-(5-(2-(2-amino-5-methylpyridin-3-yl)-5-(1H-pyrazol-1-yl)-3H-imidazo[4,5-b]pyridin-3-yl)-2,3-dihydro-1H-inden-1-yl)-3-formyl-4-hydroxybenzamide

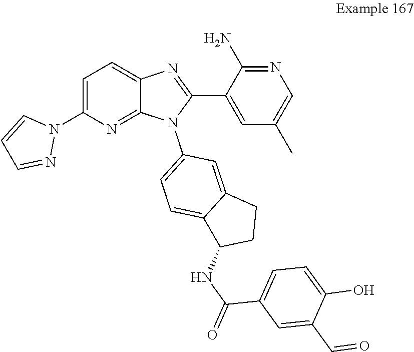 [SMILES: Cc1cnc(N)c(-c2nc3ccc(-n4cccn4)nc3n2-c2ccc3c(c2)CC[C@@H]3NC(=O)c2ccc(O)c(C=O)c2)c1]

Example 167 was prepared in a manner analogous to Example 33 (via Intermediate 33-1) starting from Intermediate 167-1 in place of Intermediate 18-2, using a reaction time of 2 h instead of overnight for the EPhos coupling, PyBOP in place of HATU and Intermediate 57-2 in place of Intermediate 5-1. MS (ESI) calcd. for C₃₂H₂₆N₈O₃: 570.21 m/z, found 571.21 [M+H]⁺. ¹H NMR (400 MHz, DMSO-d₆) δ (ppm): 10.29 (s, 1H), 8.94-9.02 (m, 1H), 8.32-8.41 (m, 2H), 8.27-8.30 (m, 1H), 8.07-8.13 (m, 1H), 7.94-8.02 (m, 1H), 7.79-7.88 (m, 2H), 7.33-7.41 (m, 2H), 7.26-7.31 (m, 1H), 7.15-7.18 (m, 1H), 7.02-7.09 (m, 1H), 6.56-6.62 (m, 1H), 5.57-5.66 (m, 1H), 3.03-3.14 (m, 1H), 2.84-2.96 (m, 1H), 2.52-2.58 (m, 1H), 2.07-2.16 (m, 1H), 1.95 (s, 3H).

### Intermediate 167-1: (S)—N-(5-(2-(2-amino-5-methylpyridin-3-yl)-5-chloro-3H-imidazo[4,5-b]pyridin-3-yl)-2,3-dihydro-1H-inden-1-yl)acetamide

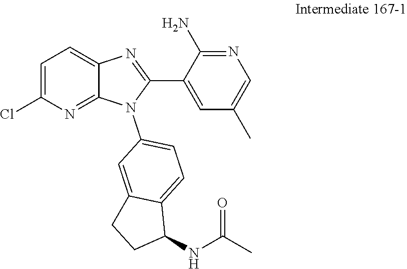 [SMILES: CC(=O)N[C@H]1CCc2cc(-n3c(-c4cc(C)cnc4N)nc4ccc(Cl)nc43)ccc21]

Intermediate 167-1 was prepared in a manner analogous to Intermediate 163-2 using 2-amino-5-methylnicotinaldehyde in place of 2-amino-5-fluoronicotinaldehyde. MS (ESI) calcd. for C₂₃H₂₁ClN₆O: 432.15 m/z, found 433.15 [M+H]⁺.

### Example 168: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-(difluoromethoxy)-5-formyl-4-hydroxybenzamide

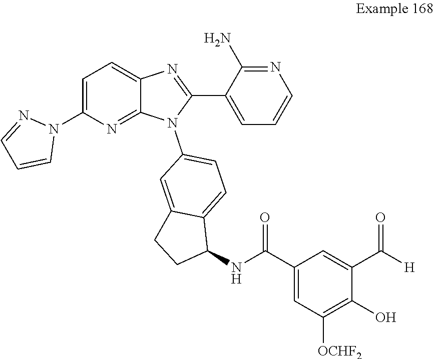 [SMILES: Nc1ncccc1-c1nc2ccc(-n3cccn3)nc2n1-c1ccc2c(c1)CC[C@@H]2NC(=O)c1cc(C=O)c(O)c(OC(F)F)c1]

### Synthetic Route:

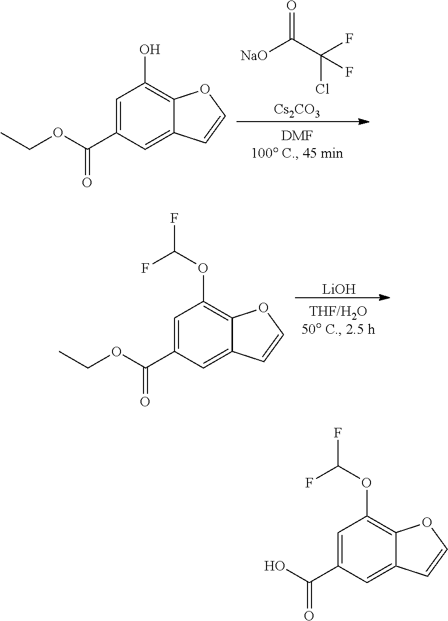

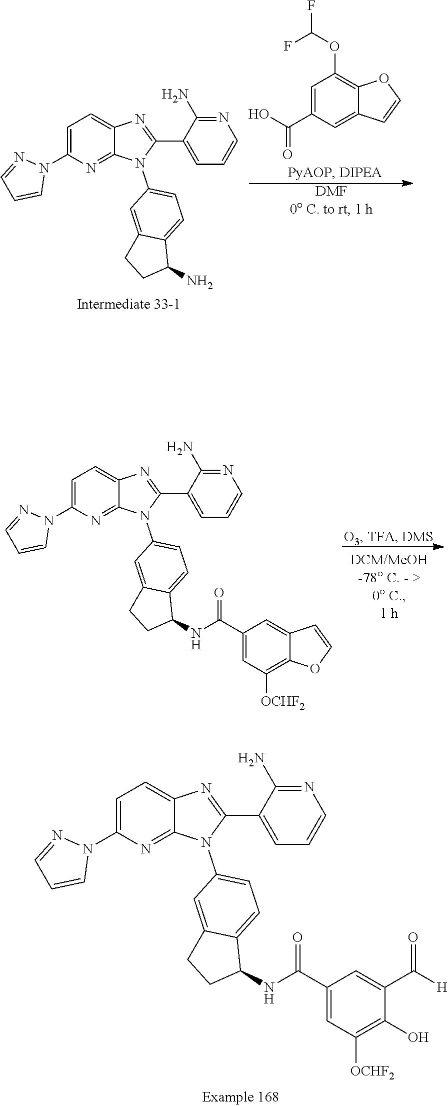 [SMILES: Nc1ncccc1-c1nc2ccc(-n3cccn3)nc2n1-c1ccc2c(c1)CC[C@@H]2N ; O=C(O)c1cc(OC(F)F)c2occc2c1 ; Nc1ncccc1-c1nc2ccc(-n3cccn3)nc2n1-c1ccc2c(c1)CC[C@@H]2NC(=O)c1cc(OC(F)F)c2occc2c1 ; Nc1ncccc1-c1nc2ccc(-n3cccn3)nc2n1-c1ccc2c(c1)CC[C@@H]2NC(=O)c1cc(C=O)c(O)c(OC(F)F)c1]

### Step 1: Synthesis of ethyl 7-(difluoromethoxy)benzofuran-5-carboxylate

To a solution of ethyl 7-hydroxybenzofuran-5-carboxylate (500 mg, 2.42 mmol, 1 equiv) in N,N-dimethylformamide (7 mL) was added cesium carbonate (1.01 g, 3.03 mmol, 1.25 equiv), followed by sodium chlorodifluoroacetate (377 mg, 2.42 mmol, 1 equiv) and the resulting mixture was heated to 100° C. and stirred for 45 min. The reaction mixture was then allowed to cool to room temperature, and the mixture was concentrated in vacuo. The residue was purified by reverse-phase flash column chromatography on C18 silica gel using a 5-100% gradient of acetonitrile in water (+10 mM ammonium formate) to afford ethyl 7-(difluoromethoxy)benzofuran-5-carboxylate (184 mg, 30%) as an off-white solid. No ionization was observed in LCMS.

### Step 2: Synthesis of 7-(difluoromethoxy)benzofuran-5-carboxylic acid

To a solution of ethyl 7-(difluoromethoxy)benzofuran-5-carboxylate (184 mg, 718 μmol, 1 equiv) in tetrahydrofuran (4.5 mL) was added lithium hydroxide (35.1 mg, 1.44 mmol, 2 equiv) and water (4.66 mL) and the mixture was heated to 50° C. and stirred for 2.5 h. The reaction mixture was then cooled to room temperature and quenched with 1 M aqueous hydrochloric acid (5 mL) and extracted with ethyl acetate (2×10 mL). The organic layers were combined, dried over sodium sulfate, filtered, and concentrated in vacuo to give 7-(difluoromethoxy)benzofuran-5-carboxylic acid (164 mg, 100% yield) as an off-white solid. MS (ESI) calculated for C₁₀H₆F₂O₄: 228.02, found 227.08 [M−H]⁻.

### Step 3: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-7-(difluoromethoxy)benzofuran-5-carboxamide

To a solution of (S)-3-(3-(1-amino-2,3-dihydro-1H-inden-5-yl)-5-(1H-pyrazol-1-yl)-3H-imidazo[4,5-b]pyridin-2-yl)pyridin-2-amine (Intermediate 33-1) (78.0 mg, 0.175 mmol, 1 equiv) and 7-(difluoromethoxy)benzofuran-5-carboxylic acid (40.0 mg, 0.175 mmol, 1 equiv) in N,N-dimethylformamide (2 mL) was added (7-azabenzotriazol-1-yloxy)tripyrrolidinophosphonium hexafluorophosphate (PyAOP) (101 mg, 0.193 mmol, 1.1 equiv) and the mixture was cooled to 0° C. N,N-diisopropylethylamine (92 μL, 0.526 mmol, 3 equiv) was added to the mixture and the resulting solution was allowed to slowly warm to room temperature and stirred for 1 h. The reaction mixture was quenched with water (5 mL) and extracted with dichloromethane (4×5 mL). The organic layers were combined, washed with brine (5 mL), dried over sodium sulfate, and concentrated in vacuo. The residue was purified by silica gel column chromatography using a 75-100% gradient of ethyl acetate in heptanes to obtain (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-7-(difluoromethoxy)benzofuran-5-carboxamide (77 mg, 71%) as a pale orange oil. MS (ESI) calculated for C₃₃H₂₄F₂N₈O₃: 618.19, found 619.32 [M+H]⁺.

### Step 4: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-(difluoromethoxy)-5-formyl-4-hydroxybenzamide (Example 168)

To a solution of (S)—N-(5-(2-(2-aminopyridin-3-yl)-5-(1H-pyrazol-1-yl)-3H-imidazo[4,5-b]-pyridin-3-yl)-2,3-dihydro-1H-inden-1-yl)-7-(difluoromethoxy)benzofuran-5-carboxamide (77 mg, 0.124 mmol, 1 equiv) in dichloromethane (5.40 mL) and methanol (540 μL) was added trifluoroacetic acid (20.0 μL, 0.259 mmol, 2 equiv) and the resulting solution was cooled to −78° C. Ozone was then bubbled through the cold solution for 15 min. The reaction mixture was bubbled with nitrogen for 5 min then quenched with dimethyl sulfide (20.0 μL, 0.270 mmol, 2 equiv) and allowed to reach 0° C. and stirred for 1 h. The reaction mixture was then concentrated in vacuo, coevaporating with dichloromethane (5×5 mL). The residue was purified by preparative HPLC using a 15-35% gradient of acetonitrile in 0.1% aqueous formic acid to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-3-(difluoro-methoxy)-5-formyl-4-hydroxybenzamide (Example 168) (13.5 mg, 17% yield) as yellow solid. MS (ESI) calculated for C₃₂H₂₄F₂N₈O₄: 622.19, found 623.25 [M+H]⁺. ¹H NMR (400 MHz, Acetic acid-d₄) δ (ppm): 10.04 (s, 1H), 8.46 (d, J=2.4 Hz, 1H), 8.42 (d, J=8.7 Hz, 1H), 8.37 (d, J=2.0 Hz, 1H), 8.15 (d, J=5.5 Hz, 1H), 8.09 (dd, J=8.7, 5.1 Hz, 2H), 7.86 (s, 1H), 7.77 (d, J=7.5 Hz, 1H), 7.61 (d, J=8.0 Hz, 1H), 7.47 (s, 1H), 7.44 (d, J=7.9 Hz, 1H), 6.86 (t, J=74.0 Hz (OCHF2), 1H), 6.78 (t, J=6.8 Hz, 1H), 6.52 (d, J=1.8 Hz, 1H), 5.87 (t, J=7.8 Hz, 1H), 3.14 (dd, J=16.3, 5.8 Hz, 1H), 3.02 (dt, J=16.3, 8.2 Hz, 1H), 2.74 (td, J=8.1, 4.4 Hz, 1H), 2.24-2.15 (m, 1H). ¹⁹F NMR (376 MHz, Acetic acid) δ (ppm): −83.00 (d, J=74.0 Hz).

### Example 169: N-[(1S)-5-[2-(2-aminopyridin-3-yl)-5-(pyrazol-1-yl)imidazo[4,5-b]pyridin-3-yl]-2,3-dihydro-1H-inden-1-yl]-7-formyl-8-hydroxyimidazo[1,2-a]pyridine-5-carboxamide

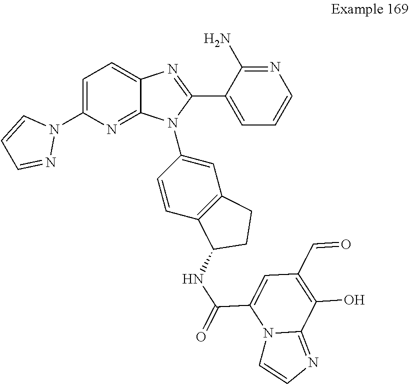 [SMILES: Nc1ncccc1-c1nc2ccc(-n3cccn3)nc2n1-c1ccc2c(c1)CC[C@@H]2NC(=O)c1cc(C=O)c(O)c2nccn12]

Example 169 was prepared in a manner analogous to Example 54 using Intermediate 33-1 in place of Intermediate 1-3, Intermediate 169-1 in place of Intermediate 54-1 and EDCI in place of DIC. MS (ESI) calcd. for C₃₂H₂₄N₁₀O₃: 596.20 m/z, found 597.25 [M+H]⁺. ¹H NMR (400 MHz, DMSO-d₆) δ ppm: 9.78 (s, 1H), 8.42-8.43 (m, 1H), 8.27-8.33 (m, 2H), 7.92-8.01 (m, 1H), 7.85-7.97 (m, 1H), 7.73-7.81 (m, 1H), 7.57-7.65 (m, 1H), 7.36-7.44 (m, 2H), 7.22-7.33 (m, 3H), 6.49-6.52 (m, 2H), 5.51-5.53 (m, 1H), 2.97-3.00 (m, 1H), 2.86-2.88 (m, 1H), 2.45-2.46 (m, 1H), 1.93-2.08 (m, 1H).

### Intermediate 169-1: 7-formyl-8-hydroxyimidazo[1,2-a]pyridine-5-carboxylic acid

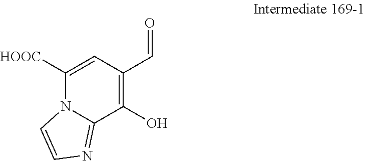 [SMILES: O=Cc1cc(C(=O)O)n2ccnc2c1O]

### Synthetic Route:

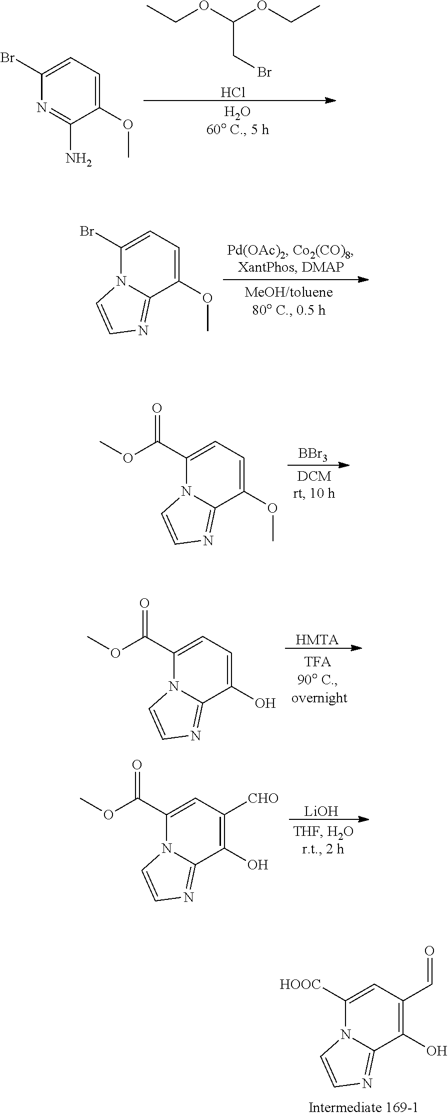

### Step 1: Synthesis of 5-bromo-8-methoxyimidazo[1,2-a]pyridine

To a solution of 2-bromo-1,1-diethoxyethane (2.91 g, 14.8 mmol, 2 equiv) in water (7.5 mL) was added hydrochloric acid (1.5 mL, concentrated) and the resulting solution was stirred at 50° C. for 3 h. The solution was cooled to 0° C. and brought to pH-9 with saturated aqueous sodium bicarbonate. To the resulting suspension was added 6-bromo-3-methoxypyridin-2-amine (1.5 g, 7.4 mmol, 1 equiv) and the resulting suspension was stirred at 65° C. for 2 h. The mixture was cooled down to room temperature and the pH was brough to 10 with 10% aqueous sodium hydroxide. The precipitated solids were collected by filtration and washed with water giving 5-bromo-8-methoxyimidazo[1,2-a]pyridine (1.2 g, 72% yield) as light-brown solid. MS (ESI) calcd. for C₈H₇BrN₂O: 225.97 m/z, found 226.95 [M+H]⁺.

### Step 2: Synthesis of methyl 8-methoxyimidazo[1,2-a]pyridine-5-carboxylate

To a solution of 5-bromo-8-methoxyimidazo[1,2-a]pyridine (500 mg, 2.20 mmol, 1 equiv) in methanol (4 mL) and toluene (12 mL) was added palladium (II) acetate (25 mg, 0.11 mmol, 0.05 equiv), XantPhos (127 mg, 0.220 mmol, 0.1 equiv), 4-dimethylaminopyridine (538 mg, 4.40 mmol, 2 equiv) and dicobalt octacarbonyl (376 mg, 1.10 mmol, 0.5 equiv). The resulting mixture was stirred for 0.5 h at 80° C. under a nitrogen atmosphere. The reaction cooled to 0° C. and quenched by the addition of saturated aqueous ammonium chloride (10 mL). The resulting mixture was extracted with ethyl acetate (30 mL×3). The combined organic layers were washed with water (20 mL×3), dried over anhydrous sodium sulfate, filtered and concentrated under reduced pressure. The residue was purified by silica gel column chromatography using a 0-80% gradient of ethyl acetate in petroleum ether to afford methyl 8-methoxyimidazo[1,2-a]pyridine-5-carboxylate (300 mg, 66% yield) as a light-yellow solid. MS (ESI) calcd. for C₁₀H₁₀N₂O₃: 206.07 m/z, found 207.10 [M+H]⁺.

### Step 3: Synthesis of methyl 8-hydroxyimidazo[1,2-a]pyridine-5-carboxylate

To a cooled (0° C.) solution of methyl 8-methoxyimidazo[1,2-a]pyridine-5-carboxylate (200 mg, 0.970 mmol, 1 equiv) in dichloromethane (10 mL) was added dropwise boron tribromide (1M in dichloromethane, 20 mL, 20 mmol, 20 equiv) and the resulting mixture was stirred at room temperature for 10 h. The mixture was concentrated and purified by silica gel column chromatography, eluting with 10% methanol in dichloromethane to afford methyl 8-hydroxyimidazo[1,2-a]pyridine-5-carboxylate (50 mg, 27% yield) as a grey-white solid. MS (ESI) calcd. for C₉H₈N₂O₃: 192.05 m/z, found 193.15 [M+H]⁺.

### Step 4: Synthesis of methyl 7-formyl-8-hydroxyimidazo[1,2-a]pyridine-5-carboxylate

To a solution of methyl 8-hydroxyimidazo[1,2-a]pyridine-5-carboxylate (50 mg, 0.26 mmol, 1 equiv) in 2,2,2-trifluoroacetic acid (2 mL) was added 1,3,5,7-tetraazaadamantane (55 mg, 0.39 mmol, 1.5 equiv) and the resulting solution was stirred overnight at 90° C. The mixture was partially concentrated under reduced pressure and diluted with water to give a suspension. The suspended solids were collected by filtration and washed with water giving methyl 7-formyl-8-hydroxyimidazo[1,2-a]pyridine-5-carboxylate (40 mg, 70% yield) as a light-yellow solid. MS (ESI) calcd. for C₁₀H₈N₂O₄: 220.05 m/z, found 221.10 [M+H]⁺.

### Step 5: Synthesis of 7-formyl-8-hydroxyimidazo[1,2-a]pyridine-5-carboxylic acid (Intermediate 169-1)

To a cooled (0° C.) solution of methyl 7-formyl-8-hydroxyimidazo[1,2-a]pyridine-5-carboxylate (30 mg, 0.14 mmol, 1 equiv) in tetrahydrofuran (4 mL) was added a solution of lithium hydroxide (10 mg, 0.41 mmol, 3 equiv) in water (1 mL) and the obtained solution was stirred for 2 h at room temperature. The resulting solution was concentrated under reduced pressure to remove tetrahydrofuran and acidified to pH˜6 with 2N hydrochloric acid. The resulting suspended solids were collected by filtration and rinsed with water to provide 7-formyl-8-hydroxyimidazo[1,2-a]pyridine-5-carboxylic acid (Intermediate 169-1) (20 mg, 71% yield) as a white solid. MS (ESI) calcd. for C₉H₆N₂O₄: 206.03 m/z, found 207.10 [M+H]⁺.

### Example 170: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-7-formyl-3-methyl-2-oxo-2,3-dihydro-1H-benzo[d]imidazole-5-carboxamide

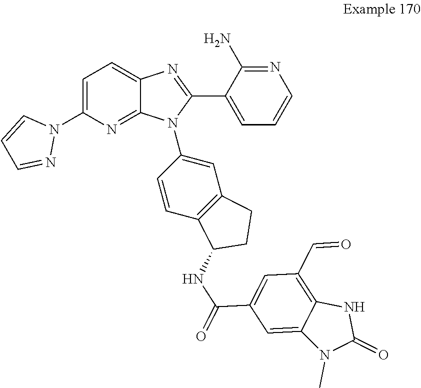 [SMILES: Cn1c(=O)[nH]c2c(C=O)cc(C(=O)N[C@H]3CCc4cc(-n5c(-c6cccnc6N)nc6ccc(-n7cccn7)nc65)ccc43)cc21]

Example 170 was prepared in a manner analogous to Example 7 using Intermediate 33-1 in place of Intermediate 1-2 and Intermediate 170-1 in place of Intermediate 5-1. MS (ESI) calcd. for C₃₃H₂₆N₁₀O₃: 610.22 m/z, found 611.30 [M+H]⁺. ¹H-NMR (400 MHz, DMSO-d₆) δ (ppm): 10.14 (s, 1H), 8.34-8.37 (m, 2H), 8.19 (s, 1H), 8.01-8.03 (m, 1H), 7.93-7.96 (m, 2H), 7.81 (s, 1H), 7.39-7.41 (m, 2H), 7.29-7.33 (m, 2H), 6.56-6.57 (m, 1H), 6.49-6.52, 1H), 5.67-5.70 (m, 1H), 3.38 (s, 3H), 3.05-3.08 (m, 1H), 2.92-2.96 (m, 1H), 2.59-2.60 (m, 1H), 2.11-2.16 (m, 1H).

### Intermediate 170-1: 7-(1,3-dioxolan-2-yl)-3-methyl-2-oxo-1-((2-(trimethylsilyl)ethoxy)methyl)-2,3-dihydro-1H-benzo[d]imidazole-5-carboxylic acid

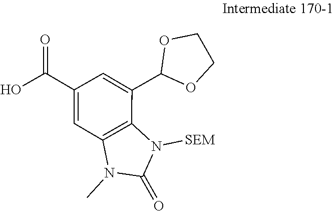 [SMILES: Cn1c(=O)n(COCC[Si](C)(C)C)c2c(C3OCCO3)cc(C(=O)O)cc21]

### Synthetic Route:

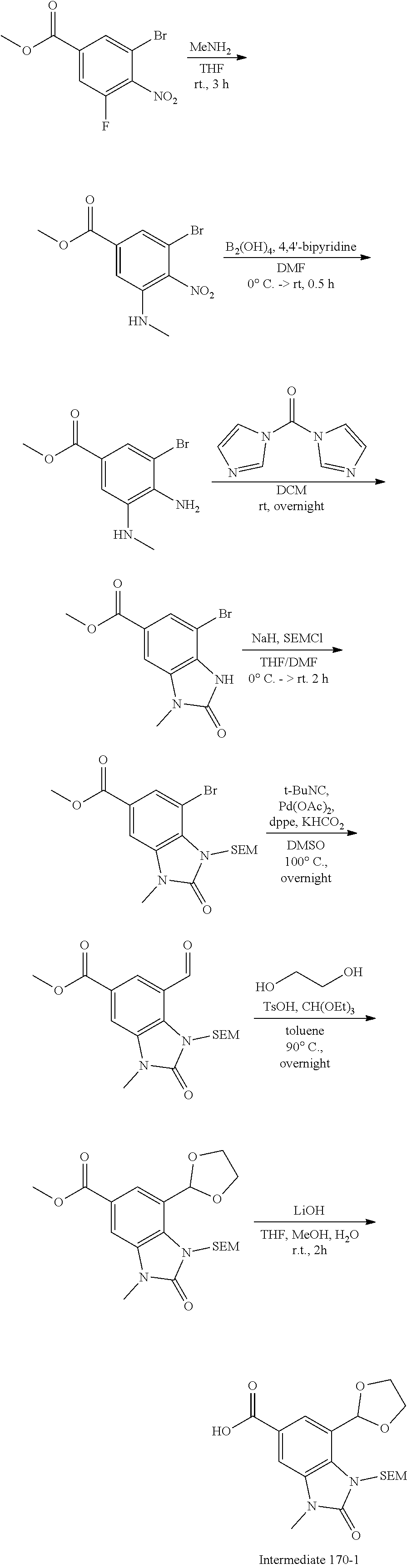

### Step 1: Synthesis of methyl 3-bromo-5-(methylamino)-4-nitrobenzoate

A mixture of methyl 3-bromo-5-fluoro-4-nitrobenzoate (4.5 g, 16 mmol, 1 equiv) and methylamine (2M in tetrahydrofuran, 16 mL, 32 mmol, 2 equiv) in tetrahydrofuran (40 mL) was stirred at room temperature for 3 h. Water (20 mL) was added and the mixture was extracted with ethyl acetate (20 mL×3). The combined organic phases were washed with brine (30 mL×3), dried with sodium sulfate, filtered and concentrated. The residue was purified by silica gel column chromatography eluting with 20% ethyl acetate in petroleum ether to afford methyl 3-bromo-5-(methylamino)-4-nitrobenzoate (4 g, 83% yield) as a red solid. MS (ESI) calcd. for C₉H₉BrN₂O₄: 287.97 m/z, found 288.95 [M+H]⁺, 290.95 [M+H+2]⁺.

### Step 2: Synthesis of methyl 4-amino-3-bromo-5-(methylamino)benzoate

To a cooled (0° C.) solution of methyl 3-bromo-5-(methylamino)-4-nitrobenzoate (4.0 g, 15 mmol, 1 equiv) 4,4′-bipyridine (0.12 g, 0.75 mmol, 0.05 equiv) in N,N-dimethylformamide (30 mL) was added tetrahydroxydiboron (5.40 g, 60.2 mmol, 4 equiv) and the resulting mixture was stirred at room temperature for 0.5 h. Water (30 mL) was added and the mixture was extracted with ethyl acetate (30 mL×3). The combined organic phases were washed with brine (30 mL×3), dried with sodium sulfate, filtered, and concentrated. The residue was purified by silica gel column chromatography eluting with 30% ethyl acetate in petroleum ether to afford methyl 4-amino-3-bromo-5-(methylamino) benzoate (3.2 g, 81% yield) as a yellow solid. MS (ESI) calcd. for C₉H₁₁BrN₂O₂: 258.00 m/z, found 258.95 [M+H]⁺, 260.95 [M+H+2]⁺.

### Step 3: Synthesis of methyl 7-bromo-3-methyl-2-oxo-2,3-dihydro-1H-benzo[d]imidazole-5-carboxylate

To a solution of methyl 4-amino-3-bromo-5-(methylamino) benzoate (2.83 g, 10.9 mmol, 1 equiv) in dichloromethane (10 mL) was added carbonyldiimidazole (2.66 g, 16.4 mmol, 1.5 equiv) and the mixture was stirred at room temperature overnight. The precipitated solids were collected by filtration and washed with 1:1 dichloromethane/methyl tert-butyl ether (3×15 mL) to provide methyl 7-bromo-3-methyl-2-oxo-2,3-dihydro-1H-benzo[d]imidazole-5-carboxylate (2.1 g, 47% yield) as a white solid. MS (ESI) calcd. for C₁₀H₉BrN₂O₃: 283.98 m/z, found 284.90 [M+H]⁺, 286.95 [M+H+2]⁺.

### Step 4: Synthesis of methyl 7-bromo-3-methyl-2-oxo-1-((2-(trimethylsilyl)ethoxy)methyl)-2,3-dihydro-1H-benzol[d]imidazole-5-carboxylate

To a cooled (0° C.) mixture of methyl 7-bromo-3-methyl-2-oxo-2,3-dihydro-1H-benzo[d]imidazole-5-carboxylate (2 g, 7 mmol, 1 equiv) in tetrahydrofuran (20 mL) and N,N-dimethylformamide (20 mL) was added 60% sodium hydride (0.25 g, 11 mmol, 1.5 equiv). The mixture was stirred at room temperature for 30 min then cooled to 0° C. (2-(chloromethoxy)ethyl)trimethylsilane (1.87 g, 11 mmol, 1.5 equiv) was added and the mixture was stirred at room temperature for 2 h. Water (20 mL) was added and the mixture was extracted with ethyl acetate (20 mL×3). The combined organic phases were washed with brine (30 mL×3), dried with sodium sulfate, filtered and concentrated. The residue was purified by silica gel column chromatography using a 0-20% gradient of ethyl acetate in petroleum ether to afford methyl 7-bromo-3-methyl-2-oxo-1-((2-(trimethylsilyl)ethoxy)methyl)-2,3-dihydro-1H-benzo[d]imidazole-5-carboxylate (1.8 g, 52% yield) as a yellow oil. MS (ESI) calcd. for C₁₆H₂₃BrNa₂O₄Si: 414.06 m/z, found 415.00 [M+H]⁺, 417.00 [M+H+2]⁺.

### Step 5: Synthesis of methyl 7-formyl-3-methyl-2-oxo-1-((2-(trimethylsilyl)ethoxy)methyl)-2,3-dihydro-1H-benzol[d]imidazole-5-carboxylate

A mixture of methyl 7-bromo-3-methyl-2-oxo-1-((2-(trimethylsilyl)ethoxy)methyl)-2,3-dihydro-1H-benzo[d]imidazole-5-carboxylate (1.5 g, 3.6 mmol, 1 equiv), tert-butyl isocyanide (0.45 g, 5.4 mmol, 1.5 equiv), palladium (II) acetate (0.08 g, 0.36 mmol, 0.1 equiv), potassium formate (0.61 g, 7.2 mmol, 2 equiv) and 1,2-bis(diphenylphosphanyl)ethane (0.29 g, 0.72 mmol, 0.1 equiv) in DMSO (30 mL) was stirred at 100° C. overnight under nitrogen atmosphere. The reaction mixture was then cooled to room temperature and quenched by the addition of saturated aqueous ammonium chloride (1 mL). Water (20 mL) was added and the mixture was extracted with ethyl acetate (20 mL×3). The combined organic phases were washed with brine (30 mL×3), dried with sodium sulfate, filtered, and concentrated. The residue was purified by silica gel column chromatography using a 0-35% gradient of ethyl acetate in petroleum ether to afford methyl 7-formyl-3-methyl-2-oxo-1-((2-(trimethylsilyl)ethoxy)methyl)-2,3-dihydro-1H-benzo[d]imidazole-5-carboxylate (0.44 g, 28%) as a yellow solid. MS (ESI) calcd. for C₁₇H₂₄N₂O₅Si: 364.15 m/z, found 363.05 [M−H]⁻.

### Step 6: Synthesis of methyl 7-(1,3-dioxolan-2-yl)-3-methyl-2-oxo-1-((2-(trimethylsilyl)ethoxy)methyl)-2,3-dihydro-1H-benzo[d]imidazole-5-carboxylate

A mixture of methyl 7-formyl-3-methyl-2-oxo-1-((2-(trimethylsilyl)ethoxy)methyl)-2,3-dihydro-1H-benzo[d]imidazole-5-carboxylate (440 mg, 1.2 mmol, 1 equiv), ethylene glycol (375 mg, 6.04 mmol, 5 equiv), p-toluenesulfonic acid (18 mg, 0.12 mmol, 0.1 equiv) and triethyl orthoformate (624 mg, 3.62 mmol, 3 equiv) in toluene (25 mL) was stirred at 110° C. overnight. The mixture was cooled to room temperature and water (20 mL) was added. The mixture was extracted with ethyl acetate (20 mL×3). The combined organic phases were washed with brine (30 mL×3), dried with sodium sulfate, filtered, and concentrated. The residue was purified by silica gel column chromatography using a 0-35% gradient of ethyl acetate in petroleum ether to afford methyl 7-(1,3-dioxolan-2-yl)-3-methyl-2-oxo-1-((2-(trimethylsilyl)ethoxy)methyl)-2,3-dihydro-1H-benzo[d]imidazole-5-carboxylate (440 mg, 71% yield) as a yellow solid. MS (ESI) calcd. for C₁₉H₂₈N₂O₆Si: 408.17 m/z, found 409.15 [M+H]⁺.

### Step 7: Synthesis of 7-(1,3-dioxolan-2-yl)-3-methyl-2-oxo-1-((2-(trimethylsilyl)ethoxy)methyl)-2,3-dihydro-1H-benzo[d]imidazole-5-carboxylic acid (Intermediate 170-1)

To a solution of methyl 7-(1,3-dioxolan-2-yl)-3-methyl-2-oxo-1-((2-(trimethylsilyl)ethoxy)methyl)-2,3-dihydro-1H-benzo[d]imidazole-5-carboxylate (365 mg, 0.893 mmol, 1 equiv) in methanol (1.8 mL) and tetrahydrofuran (4 mL) was added 2M aqueous lithium hydroxide (1.79 mL, 3.57 mmol, 4 equiv). The mixture was stirred at room temperature for 2 h then concentrated under reduced pressure to remove methanol and tetrahydrofuran. The pH of the mixture was brought to ˜6 with 2N hydrochloric acid. The precipitated solids were collected by filtration and rinsed with water to afford 7-(1,3-dioxolan-2-yl)-3-methyl-2-oxo-1-((2-(trimethylsilyl)ethoxy)methyl)-2,3-dihydro-1H-benzo[d]imidazole-5-carboxylic acid (Intermediate 170-1) (350 mg, 99% yield) as a white solid. MS (ESI) calcd. for C₁₈H₂₆N₂O₆Si: 394.16 m/z, found 395.05 [M+H]⁺.

### Example 171: N-[(1S)-5-[2-(2-aminopyridin-3-yl)-5-[1-(oxetan-3-yl)pyrazol-3-yl]imidazo[4,5-b]pyridin-3-yl]-2,3-dihydro-1H-inden-1-yl]-3-formyl-4-hydroxybenzamide

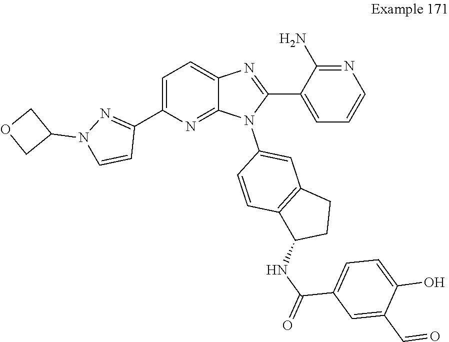 [SMILES: Nc1ncccc1-c1nc2ccc(-c3ccn(C4COC4)n3)nc2n1-c1ccc2c(c1)CC[C@@H]2NC(=O)c1ccc(O)c(C=O)c1]

### Synthetic Route:

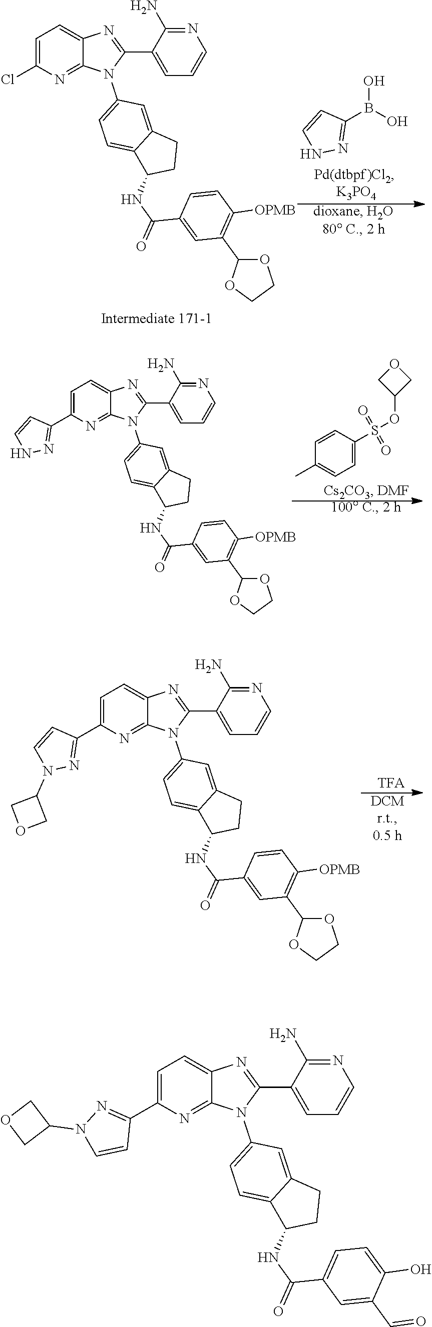

### Step 1: Synthesis of N-[(1S)-5-[2-(2-aminopyridin-3-yl)-5-(1H-pyrazol-3-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

A mixture of N-[(1S)-5-[2-(2-aminopyridin-3-yl)-5-chloroimidazo[4,5-b]pyridin-3-yl]-2,3-dihydro-1H-inden-1-yl]-3-(1,3-dioxolan-2-yl)-4-[(4-methoxyphenyl)methoxy]benzamide (Intermediate 160-1) (90 mg, 0.13 mmol, 1 equiv), 1H-pyrazol-3-ylboronic acid (44 mg, 0.39 mmol, 3 equiv), bis(di-tert-butylphosphino)ferrocene]dichloropalladium(II) (17 mg, 0.026 mmol, 0.2 equiv) and tribasic potassium phosphate (83 mg, 0.39 mmol, 3 equiv) in 1,4-dioxane (6 mL) and water (1.5 mL) was stirred for 2 h at 80° C. under nitrogen atmosphere. The mixture was purified by reverse-phase flash column chromatography on C18 silica gel using a 20-70% gradient of acetonitrile in water (+0.05% ammonium bicarbonate) to afford N-[(1S)-5-[2-(2-aminopyridin-3-yl)-5-(1H-pyrazol-3-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (90 mg, 89%) as a yellow solid. MS (ESI) calcd. for C₄₁H₃₆N₈O₅: 720.28 m/z, found 721.30 [M+H]⁺.

### Step 2: Synthesis of N-[(1S)-5-[2-(2-aminopyridin-3-yl)-5-[1-(oxetan-3-yl)pyrazol-3-yl]imidazo[4,5-b]pyridin-3-yl]-2,3-dihydro-1H-inden-1-yl]-3-(1,3-dioxolan-2-yl)-4-[(4-methoxyphenyl)methoxy]benzamide

To a stirred solution of N-[(1S)-5-[2-(2-aminopyridin-3-yl)-5-(1H-pyrazol-3-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (90 mg, 0.125 mmol, 1 equiv) and cesium carbonate (81 mg, 0.25 mmol, 2 equiv) in N,N-dimethylformamide (3 mL) was added oxetan-3-yl 4-methylbenzenesulfonate (34 mg, 0.15 mmol, 1.2 equiv) in portions and the resulting mixture was stirred at 100° C. 2 h. The mixture was purified by reverse-phase flash column chromatography on C18 silica gel using a 20-70% gradient of acetonitrile in water (+0.05% ammonium bicarbonate) to afford N-[(1S)-5-[2-(2-aminopyridin-3-yl)-5-[1-(oxetan-3-yl)pyrazol-3-yl]imidazo[4,5-b]pyridin-3-yl]-2,3-dihydro-1H-inden-1-yl]-3-(1,3-dioxolan-2-yl)-4-[(4-methoxyphenyl)methoxy]benzamide (55 mg, 51% yield) as a yellow solid. MS (ESI) calcd. for C₄₄H₄₀N₈O₆: 776.31 m/z, found 777.30 [M+H]⁺.

### Step 3: Synthesis of N-[(1S)-5-[2-(2-aminopyridin-3-yl)-5-[1-(oxetan-3-yl)pyrazol-3-yl]imidazo[4,5-b]pyridin-3-yl]-2,3-dihydro-1H-inden-1-yl]-3-formyl-4-hydroxybenzamide

To a solution of (S)—N-(5-(2-(2-aminopyridin-3-yl)-5-(1-(oxetan-3-yl)-1H-pyrazol-3-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide (50 mg, 0.067 mmol, 1 equiv) in dichloromethane (5 mL) was added 2,2,2-trifluoroacetic acid (0.5 mL) and the solution was stirred for 0.5 h at room temperature. The mixture was brought to pH 8-9 with saturated aqueous sodium bicarbonate. The resulting mixture was extracted with ethyl acetate (100 mL×3). The combined organic layers were washed with water (100 mL×3), dried over anhydrous sodium sulfate, filtered and concentrated under reduced pressure. The crude product was purified by preparative HPLC on a XBridge Shield RP18 OBD Column using a 14-44% gradient of acetonitrile in water (+10 mmol/L ammonium bicarbonate) to afford N-[(1S)-5-[2-(2-aminopyridin-3-yl)-5-[1-(oxetan-3-yl)pyrazol-3-yl]imidazo[4,5-b]pyridin-3-yl]-2,3-dihydro-1H-inden-1-yl]-3-formyl-4-hydroxybenzamide (Example 171) (6.1 mg, 15% yield) as a light-yellow solid. MS (ESI) calcd. for C₃₄H₂₈N₈O₄: 612.22 m/z, found 613.30 [M+H]⁺. ¹H-NMR (400 MHz, DMSO-d₆) δ (ppm): 10.18 (s, 1H), 8.19-8.34 (m, 2H), 7.85-7.96 (m, 4H), 7.21-7.30 (m, 4H), 6.80-6.91 (m, 2H), 6.47 (s, 1H), 5.56 (br s, 2H), 4.90-4.95 (m, 4H), 2.96-3.00 (m, 2H), 2.50-2.51 (m, 1H), 2.04-2.07 (m, 1H).

### Intermediate 171-1: (S)—N-(5-(2-(2-aminopyridin-3-yl)-5-chloro-3H-imidazo[4,5-b]pyridin-3-yl)-2,3-dihydro-1H-inden-1-yl)-3-(1,3-dioxolan-2-yl)-4-((4-methoxybenzyl)oxy)benzamide

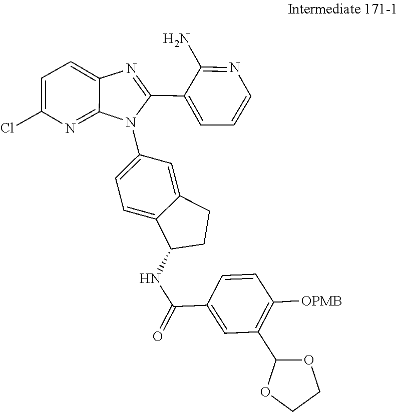

Intermediate 171-1 was prepared in a manner analogous to Intermediate 94-1 using Intermediate 5-1 in place of Intermediate 57-1 and PyBOP in place of HATU. MS (ESI) calcd. for C₃₈H₃₃ClN₆O₅: 688.22 m/z, found 689.20 [M+H]⁺.

### Example 172: (S)—N-(5-(2-(2-aminopyridin-3-yl)-5-(1H-pyrazolo[3,4-b]pyridin-1-yl)-3H-imidazo[4,5-b]pyridin-3-yl)-2,3-dihydro-1H-inden-1-yl)-2,3-difluoro-5-formyl-4-hydroxybenzamide

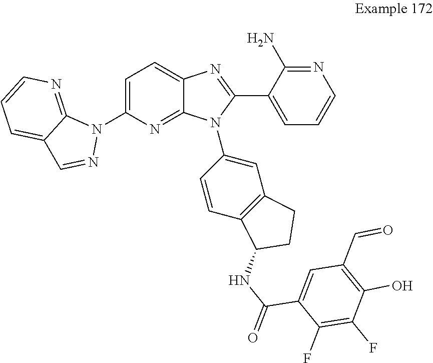 [SMILES: Nc1ncccc1-c1nc2ccc(-n3ncc4cccnc43)nc2n1-c1ccc2c(c1)CC[C@@H]2NC(=O)c1cc(C=O)c(O)c(F)c1F]

Example 172 was prepared in a manner analogous to Example 12 using Intermediate 172-1 in place of Intermediate 1-3 and Intermediate 118-1 in place of Intermediate 12-1. MS (ESI) calcd. for C₃₄H₂₃F₂N₉O₃, 643.19 m/z, found 644.25 [M+H]⁺. ¹H NMR (300 MHz, DMSO-d₆) δ (ppm): 10.15 (s, 1H), 8.52-8.75 (m, 1H), 8.27-8.47 (m, 3H), 8.02-8.22 (m, 1H), 7.87-8.02 (m, 1H), 7.62-7.75 (m, 1H), 7.15-7.45 (m, 5H), 6.35-6.54 (m, 1H), 5.35-5.61 (m, 1H), 2.77-3.05 (m, 2H), 2.36-2.51 (m, 1H), 1.87-2.13 (m, 1H).

### Intermediate 172-1: 3-{3-[(1S)-1-amino-2,3-dihydro-1H-inden-5-yl]-5-{pyrazolo[3,4-b] pyridin-1-yl}Imidazo[4,5-b]pyridin-2-yl}pyridin-2-amine

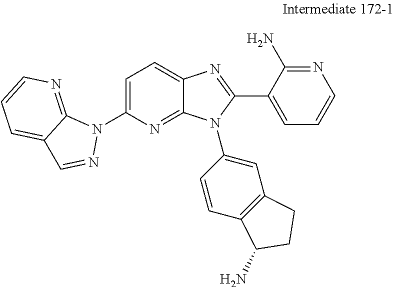 [SMILES: Nc1ncccc1-c1nc2ccc(-n3ncc4cccnc43)nc2n1-c1ccc2c(c1)CC[C@@H]2N]

Intermediate 172-1 was prepared in a manner analogous to Intermediate 33-1 using 1H-pyrazolo[3,4-b]pyridine in place of pyrazole, 1,4-dioxane/N,N-dimethylformamide (4:1) in place of 1,4-dioxane and a reaction time of 2 h instead of overnight for Step 1. MS (ESI) calcd. for C₂₆H₂₁N₉: 459.19 m/z, found 460.25 [M+H]⁺.

### Example 173: N-[(1S)-5-[2-(2-aminopyridin-3-yl)-5-(5-methyl-1,2,4-oxadiazol-3-yl)imidazo[4,5-b]pyridin-3-yl]-2,3-dihydro-1H-inden-1-yl]-3-formyl-4-hydroxybenzamide

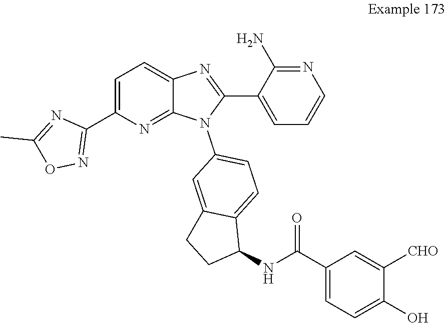 [SMILES: Cc1nc(-c2ccc3nc(-c4cccnc4N)n(-c4ccc5c(c4)CC[C@@H]5NC(=O)c4ccc(O)c(C=O)c4)c3n2)no1]

Example 173 was prepared in a manner analogous to Example 12 using Intermediate 173-2 in place of Intermediate 1-3 and Intermediate 1-5 in place of Intermediate 12-1. MS (ESI) calcd. for C₃₁H₂₄N₈O₄: 572.19 m/z, found 573.20 [M+H]⁺. ¹H NMR (400 MHz, DMSO-d₆) δ (ppm): 10.28 (s, 1H), 8.90-9.10 (m, 1H), 8.21-8.42 (m, 2H), 7.90-8.21 (m, 3H), 7.22-7.55 (m, 4H), 6.95-7.22 (m, 1H), 6.35-6.58 (m, 1H), 5.50-5.78 (m, 1H), 2.85-3.20 (m, 2H), 2.65 (s, 3H), 2.50-2.51 (m, 1H), 1.99-2.22 (m, 1H).

### Intermediate 173-2: (S)-3-(3-(1-amino-2,3-dihydro-1H-inden-5-yl)-5-(5-methyl-1,2,4-oxadiazol-3-yl)-3H-imidazo[4,5-b]pyridin-2-yl)pyridin-2-amine

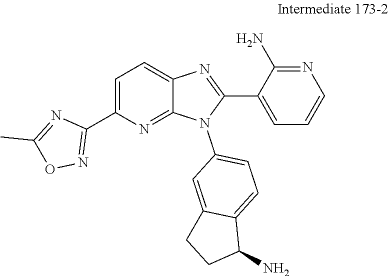 [SMILES: Cc1nc(-c2ccc3nc(-c4cccnc4N)n(-c4ccc5c(c4)CC[C@@H]5N)c3n2)no1]

### Synthetic Route:

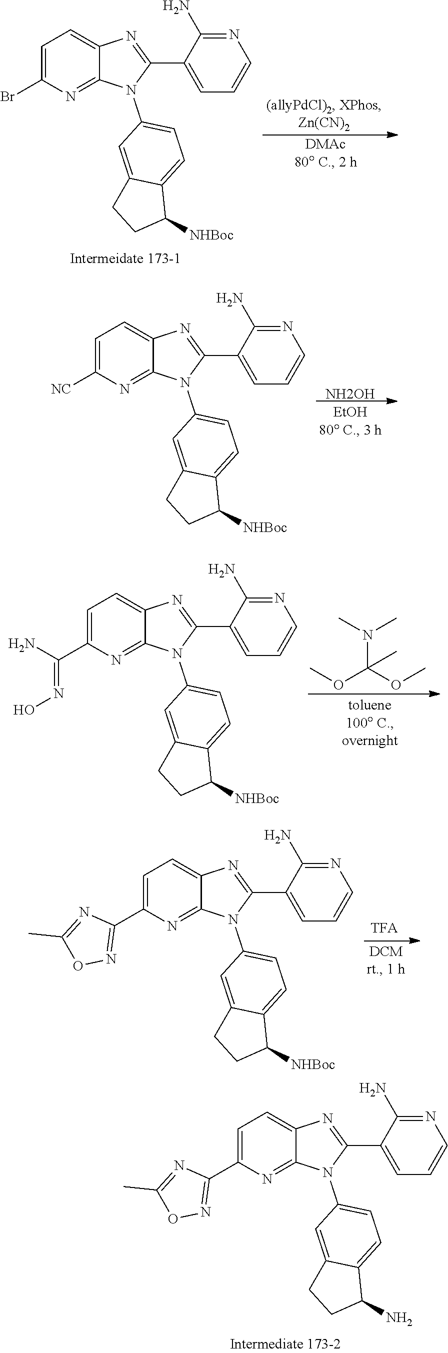

### Step 1: Synthesis of tert-butyl (S)-(5-(2-(2-aminopyridin-3-yl)-5-cyano-3H-imidazo[4,5-b]pyridin-3-yl)-2,3-dihydro-1H-inden-1-yl)carbamate

To a solution of tert-butyl N-[(1S)-5-[2-(2-aminopyridin-3-yl)-5-bromoimidazo[4,5-b]pyridin-3-yl]-2,3-dihydro-1H-inden-1-yl]carbamate (Intermediate 173-1) (1.00 g, 1.92 mmol, 1 equiv) in dimethyl acetamide (15 mL) were added allylpalladium(II) chloride dimer (70 mg, 0.19 mmol, 0.1 equiv), XPhos (91 mg, 0.19 mmol, 0.1 equiv) and Zn(CN)₂ (563 mg, 4.80 mmol, 2.5 equiv). The resulting mixture was stirred at 80° C. for 2 h. The reaction mixture was concentrated under reduced pressure and the residue was purified by silica gel column chromatography using a 0-6% gradient of methanol in dichloromethane to afford tert-butyl N-[(1S)-5-[2-(2-aminopyridin-3-yl)-5-cyanoimidazo[4,5-b]pyridin-3-yl]-2,3-dihydro-1H-inden-1-yl]carbamate (800 mg, 89% yield) as a yellow solid. MS (ESI) calcd. for C₂₆H₂₅N₇O₂: 467.21 m/z, found 468.15 [M+H]⁺.

### Step 2: Synthesis of tert-butyl N-[(1S)-5-[2-(2-aminopyridin-3-yl)-5-[(Z)—N′-hydroxycarbamimidoyl]imidazo[4,5-b]pyridin-3-yl]-2,3-dihydro-1H-inden-1-yl]carbamate

To a solution of tert-butyl N-[(1S)-5-[2-(2-aminopyridin-3-yl)-5-cyanoimidazo[4,5-b]pyridin-3-yl]-2,3-dihydro-1H-inden-1-yl]carbamate (600 mg, 1.28 mmol, 1 equiv) in ethanol (20 mL) was added hydroxylamine (50% aqueous, 0.4 mL, 4.5 equiv). The obtained mixture was stirred at 80° C. for 3 h under nitrogen atmosphere. The reaction mixture was concentrated under reduced pressure. The resulting residue was dissolved in ethyl acetate (50 mL) and washed with water (50 mL) then concentrated to afford crude tert-butyl N-[(1S)-5-[2-(2-aminopyridin-3-yl)-5-[(Z)—N′-hydroxycarbamimidoyl]imidazo[4,5-b]pyridin-3-yl]-2,3-dihydro-1H-inden-1-yl]carbamate (600 mg, 94% crude) as a light brown solid, which was used directly in the next step without any purification. MS (ESI) calcd. for C₂₆H₂₈N₈O₃: 500.23 m/z, found 501.15 [M+H]⁺.

### Step 3: Synthesis of tert-butyl N-[(1S)-5-[2-(2-aminopyridin-3-yl)-5-(5-methyl-1,2,4-oxadiazol-3-yl)imidazo[4,5-b]pyridin-3-yl]-2,3-dihydro-1H-inden-1-yl]carbamate

To a solution of tert-butyl N-[(1S)-5-[2-(2-aminopyridin-3-yl)-5-[(Z)—N′-hydroxycarbamimidoyl]imidazo[4,5-b]pyridin-3-yl]-2,3-dihydro-1H-inden-1-yl]carbamate (600 mg, 1.20 mmol, 1 equiv) in toluene (30 mL) was added (1,1-dimethoxyethyl)dimethylamine (319 mg, 2.40 mmol, 2 equiv) and the resulting solution was stirred at 100° C. overnight. The reaction mixture was concentrated to dryness and the residue was purified by silica gel column chromatography using a 0-6% gradient of methanol in dichloromethane to afford tert-butyl N-[(1S)-5-[2-(2-aminopyridin-3-yl)-5-(5-methyl-1,2,4-oxadiazol-3-yl)imidazo[4,5-b]pyridin-3-yl]-2,3-dihydro-1H-inden-1-yl]carbamate (300 mg, 48% yield) as a yellow solid. MS (ESI) calcd. for C₂₈H₂₈N₈O₃: 524.23 m/z, found 525.25 [M+H]⁺.

### Step 4: Synthesis of 3-{3-[(1S)-1-amino-2,3-dihydro-1H-inden-5-yl]-5-(5-methyl-1,2,4-oxadiazol-3-yl)imidazo[4,5-b]pyridin-2-yl}pyridin-2-amine (Intermediate 173-2)

To a solution of tert-butyl N-[(1S)-5-[2-(2-aminopyridin-3-yl)-5-(5-methyl-1,2,4-oxadiazol-3-yl)imidazo[4,5-b]pyridin-3-yl]-2,3-dihydro-1H-inden-1-yl]carbamate (290 mg, 0.553 mmol, 1 equiv) in dichloromethane (5 mL) was added 2,2,2-trifluoroacetic acid (10 mL). The resulting solution was stirred at room temperature for 1 h. The solution was concentrated under reduced pressure at 0° C. Saturated aqueous sodium bicarbonate was added and the mixture was extracted with ethyl acetate (3×20 mL). The combined organic layers were dried over anhydrous sodium sulfate, filtered and concentrated under reduced pressure to afford 3-{3-[(1S)-1-amino-2,3-dihydro-1H-inden-5-yl]-5-(5-methyl-1,2,4-oxadiazol-3-yl)imidazo[4,5-b]pyridin-2-yl}pyridin-2-amine (Intermediate 173-2) (180 mg, 77%) as a yellow solid. MS (ESI) calcd. for C₂₃H₂₀N₈O: 424.18 m/z, found 425.20 [M+H]⁺.

### Intermediate 173-1: tert-butyl (S)-(5-(2-(2-aminopyridin-3-yl)-5-bromo-3H-imidazo[4,5-b]pyridin-3-yl)-2,3-dihydro-1H-inden-1-yl)carbamate

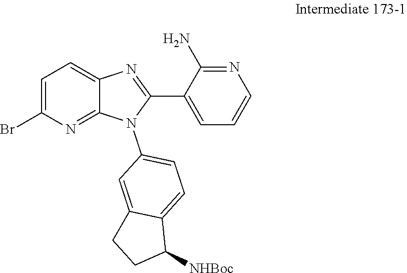 [SMILES: CC(C)(C)OC(=O)N[C@H]1CCc2cc(-n3c(-c4cccnc4N)nc4ccc(Br)nc43)ccc21]

### Synthetic Route:

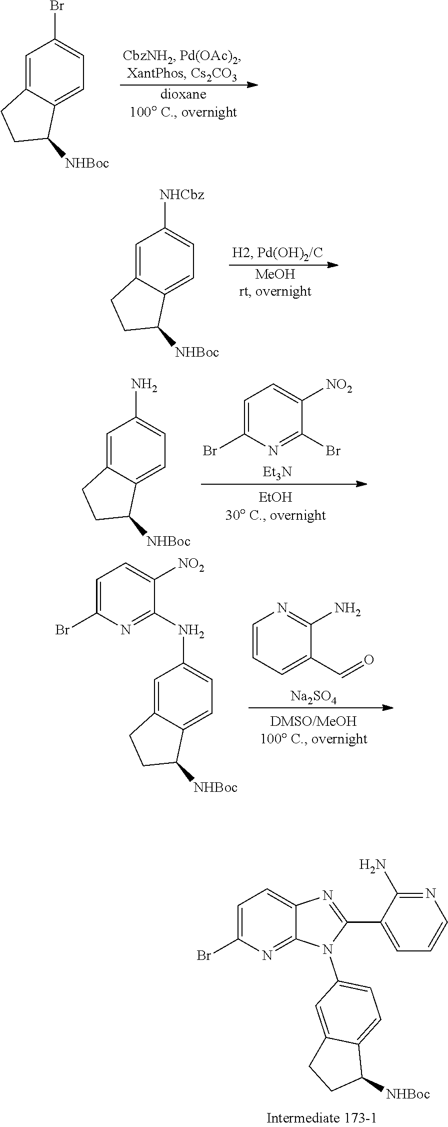

### Step 1: Synthesis of benzyl N-[(1S)-1-[(tert-butoxycarbonyl)amino]-2,3-dihydro-1H-inden-5-yl]carbamate

To a solution of tert-butyl N-[(1S)-5-bromo-2,3-dihydro-1H-inden-1-yl]carbamate (60.00 g, 192.2 mmol, 1 equiv), benzyl carbamate (34.63 g, 230.6 mmol, 1.2 equiv) and XantPhos (22.24 g, 38.44 mmol, 0.2 equiv) in 1,4-dioxane (1.2 L) were added Cs₂CO₃ (125.23 g, 384.36 mmol, 2 equiv) and Pd(OAc)₂ (4.31 g, 19.2 mmol, 0.1 equiv). After stirring overnight at 100° C. under nitrogen atmosphere the mixture was cooled to room temperature and the product was precipitated by the addition of H₂O. The precipitated solids were collected by filtration and washed with H₂O (3×400 mL) then suspended in ethyl acetate and filtered, washing with ethyl acetate. The filtrate was concentrated under reduced pressure to afford benzyl N-[(1S)-1-[(tert-butoxycarbonyl)amino]-2,3-dihydro-1H-inden-5-yl]carbamate (50 g, 46%) as a grey solid. MS (ESI) calcd. for C₂₂H₂₆N₂O₄, 382.19 m/z, found: 381.10 [M−H]⁻.

### Step 2: Synthesis of tert-butyl N-[(1S)-5-amino-2,3-dihydro-1H-inden-1-yl]carbamate

To a solution of benzyl N-[(1S)-1-[(tert-butoxycarbonyl)amino]-2,3-dihydro-1H-inden-5-yl]carbamate (50.00 g, 130.7 mmol, 1 equiv) in 500 mL CH₃OH was added 10% Pd(OH)₂/C (5.00 g, 35.6 mmol, 0.27 equiv) in a pressure tank. The mixture was hydrogenated at room temperature under 30 psi of hydrogen overnight then filtered through a Celite pad and concentrated under reduced pressure to afford tert-butyl N-[(1S)-5-amino-2,3-dihydro-1H-inden-1-yl]carbamate (40 g, 73%) as a brown solid. MS (ESI) calcd. for C₁₄H₂₀N₂O₂, 248.15 m/z, found: 249.15 [M+H]⁺.

### Step 3: Synthesis of tert-butyl N-[(1S)-5-[(6-bromo-3-nitropyridin-2-yl)amino]-2,3-dihydro-1H-inden-1-yl]carbamate

A mixture of tert-butyl N-[(1S)-5-amino-2,3-dihydro-1H-inden-1-yl]carbamate (40.0 g, 161 mmol, 1 equiv) and triethylamine (48.90 g, 483.2 mmol, 3 equiv) in EtOH (800 mL) was stirred at room temperature until dissolved. 2,6-dibromo-3-nitropyridine (54.49 g, 193.3 mmol, 1.2 equiv) was added and the mixture was stirred at 30° C. overnight. The mixture was allowed to cool to room temperature and the precipitated solids were collected by filtration and washed with EtOH (3×100 mL) to afford tert-butyl N-[(1S)-5-[(6-bromo-3-nitropyridin-2-yl)amino]-2,3-dihydro-1H-inden-1-yl]carbamate (30 g, 27.30%) as a red solid. MS (ESI) calcd. for C₁₉H₂₁BrN₄O₄, 448.07 m/z, found: 447.00 [M−H]⁻.

### Step 4: Synthesis of tert-butyl N-[(1S)-5-[2-(2-aminopyridin-3-yl)-5-bromoimidazo[4,5-b]pyridin-3-yl]-2,3-dihydro-1H-inden-1-yl]carbamate (Intermediate 173-1)

To a solution of tert-butyl N-[(1S)-5-[(6-bromo-3-nitropyridin-2-yl)amino]-2,3-dihydro-1H-inden-1-yl]carbamate (30.0 g, 66.8 mmol, 1 equiv) in DMSO (600 mL) and MeOH (100 mL) was added 2-aminopyridine-3-carbaldehyde (8.97 g, 73.4 mmol, 1.1 equiv) and the mixture was stirred until the solids were dissolved. Na₂S₂O₄ (25.57 g, 146.9 mmol, 2.2 equiv) was added and the mixture was stirred at 100° C. overnight. The product was precipitated by the addition of H₂O. The precipitated solids were collected by filtration and washed with H₂O (3×500 mL). The residue was purified by silica gel column chromatography eluting with CH₂Cl₂/MeOH (10:1) to afford tert-butyl N-[(1S)-5-[2-(2-aminopyridin-3-yl)-5-bromoimidazo[4,5-b]pyridin-3-yl]-2,3-dihydro-1H-inden-1-yl]carbamate (Intermediate 173-1) (15 g, 36%) as a yellow solid. MS (ESI) calcd. for C₂₅H₂₅BrN₆O₂, 520.12 m/z, found: 521.20 [M+H]⁺.

### Example 174: N-[(1S)-5-[2-(2-aminopyridin-3-yl)-5-(5-methyl-1,3-oxazol-2-yl)imidazo[4,5-b]pyridin-3-yl]-2,3-dihydro-1H-inden-1-yl]-3-formyl-4-hydroxybenzamide

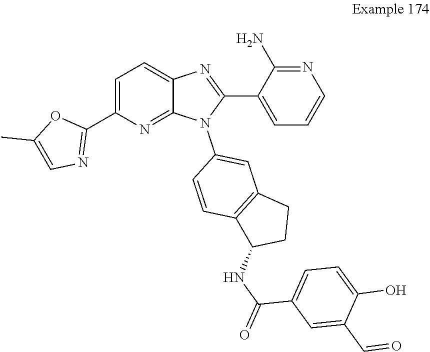 [SMILES: Cc1cnc(-c2ccc3nc(-c4cccnc4N)n(-c4ccc5c(c4)CC[C@@H]5NC(=O)c4ccc(O)c(C=O)c4)c3n2)o1]

Example 174 was prepared in a manner analogous to Example 109 using 5-methyl-2-(tributylstannyl)-1,3-oxazole in place of Intermediate 109-1 and a reaction time of 2 h instead of 4 days for Step 1. MS (ESI) calcd. for C₃₂H₂₅N₇O₄: 571.20 m/z, found 572.25 [M+H]⁺. ¹H-NMR (400 MHz, DMSO-d₆) δ (ppm): 10.25 (s, 1H), 8.28-8.32 (m, 1H), 8.12-8.27 (m, 1H), 8.05-8.11 (m, 1H), 7.90-8.04 (m, 2H), 7.25-7.41 (m, 4H), 6.80-7.08 (m, 2H), 6.40-6.49 (m, 1H), 5.58-5.68 (m, 1H), 2.96-3.05 (m, 1H), 2.81-2.95 (m, 1H), 2.42-2.59 (m, 1H), 2.31-2.40 (m, 3H), 2.02-2.18 (m, 1H).

### Example 175: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-(dimethylamino)-7-formyl-1H-benzo[d]imidazole-5-carboxamide

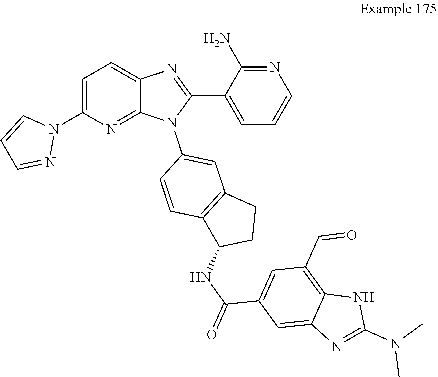 [SMILES: CN(C)c1nc2cc(C(=O)N[C@H]3CCc4cc(-n5c(-c6cccnc6N)nc6ccc(-n7cccn7)nc65)ccc43)cc(C=O)c2[nH]1]

Example 175 was prepared in a manner analogous to Example 7 using Intermediate 33-1 in place of Intermediate 1-2 and Intermediate 175-1 in place of Intermediate 5-1. MS (ESI) calcd. for C₃₄H₂₉N₁₁O₂: 623.25 m/z, found 624.25 [M+H]⁺. ¹H NMR (400 MHz, DMSO-d₆) δ (ppm): 10.51 (s, 1H), 8.31-8.34 (m, 2H), 8.06-8.07 (m, 1H), 7.98-7.99 (m, 3H), 7.91-7.94 (m, 1H), 7.34-7.36 (m, 2H), 7.25-7.28 (m, 2H), 6.53-6.54 (m, 1H), 6.45-6.48 (m, 1H), 5.61-5.65 (m, 1H), 3.14 (s, 6H), 2.84-3.09 (m, 2H), 2.50-2.51 (m, 1H), 2.02-2.10 (m, 1H).

### Intermediate 175-1: 2-(dimethylamino)-7-(1,3-dioxolan-2-yl)-1-((2-(trimethylsilyl)ethoxy)methyl)-1H-benzo[d]imidazole-5-carboxylic acid

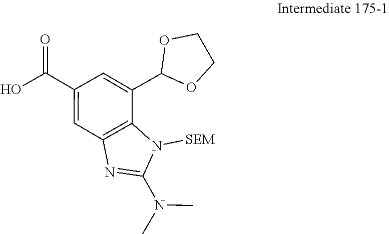 [SMILES: CN(C)c1nc2cc(C(=O)O)cc(C3OCCO3)c2n1COCC[Si](C)(C)C]

### Synthetic Route:

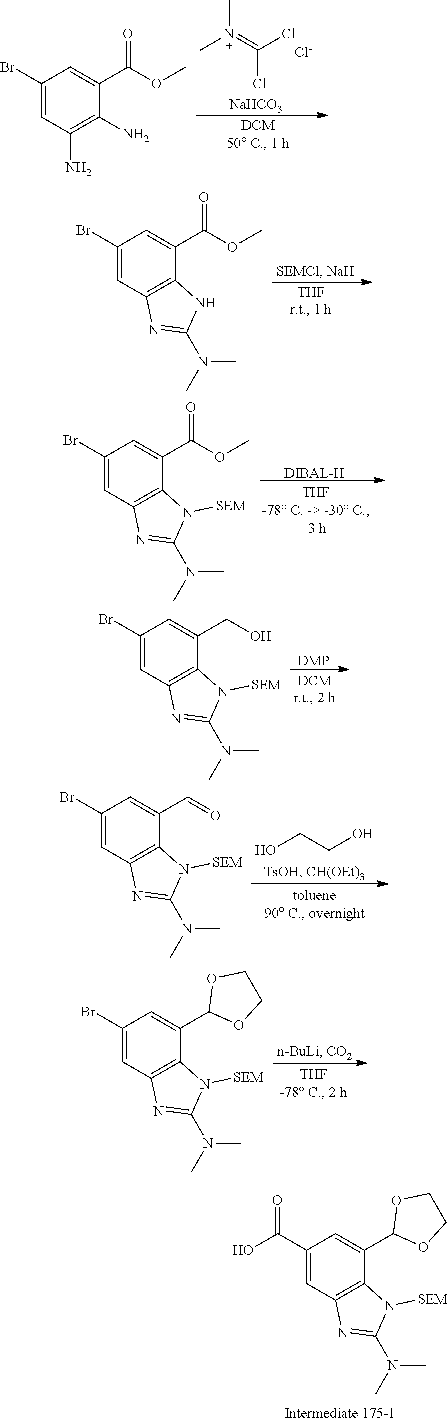

### Step 1: Synthesis of methyl 5-bromo-2-(dimethylamino)-1H-benzo[d]imidazole-7-carboxylate

A suspension of methyl 2,3-diamino-5-bromobenzoate (4.5 g, 18 mmol, 1 equiv), N-(dichloromethylene)-N-methylmethanaminium chloride (3.58 g, 22.0 mmol, 1.2 equiv) in dichloromethane (100 mL) was stirred at 50° C. for 1 h. The resulting mixture was cooled to room temperature and water (200 mL) was added. The mixture was extracted with ethyl acetate (3×200 mL). The combined organic phases were washed with brine (30 mL×3), dried with sodium sulfate, filtered and concentrated. The residue was purified by silica gel column chromatography eluting with 30% ethyl acetate in petroleum ether to afford methyl 5-bromo-2-(dimethylamino)-1H-benzo[d]imidazole-7-carboxylate (4.7 g, 86%) as a yellow solid. MS (ESI) calcd. for C₁₁H₁₂BrN₃O₂: 297.01 m/z, found 298.00 [M+H]⁺.

### Step 2: Synthesis of methyl 5-bromo-2-(dimethylamino)-1-((2-(trimethylsilyl)ethoxy)methyl)-1H-benzo[d]imidazole-7-carboxylate

To a cooled (0° C.) mixture of methyl 5-bromo-2-(dimethylamino)-1H-benzo[d]imidazole-7-carboxylate (4.7 g, 16 mmol, 1 equiv) in tetrahydrofuran (120 mL) was added 60% sodium hydride (0.57 g, 23.6 mmol, 1.5 equiv). The mixture was stirred at room temperature for 10 min then cooled to 0° C. (2-(Chloromethoxy)ethyl)trimethylsilane (4.21 g, 25.2 mmol, 1.6 equiv) was added and the mixture was stirred at room temperature for 1 h. The reaction was quenched with water (100 mL) and the mixture was extracted with ethyl acetate (100 mL×3). The combined organic phases were washed with brine (30 mL×3), dried with sodium sulfate, filtered, and concentrated. The residue was purified by silica gel column chromatography eluting with 50% ethyl acetate in petroleum ether to afford methyl 5-bromo-2-(dimethylamino)-1-((2-(trimethylsilyl)ethoxy)methyl)-1H-benzo[d]imidazole-7-carboxylate (3.5 g, 52%) as a yellow solid. MS (ESI) calcd. for C₁₇H₂₆BrN₃O₃Si: 427.09 m/z, found 428.15 [M+H]⁺.

### Step 3: Synthesis of (5-bromo-2-(dimethylamino)-1-((2-(trimethylsilyl)ethoxy)methyl)-1H-benzo[d]imidazol-7-yl)methanol

To a cooled (−78° C.) solution of methyl 5-bromo-2-(dimethylamino)-1-((2-(trimethylsilyl)ethoxy)methyl)-1H-benzo[d]imidazole-7-carboxylate (3.2 g, 7.5 mmol, 1 equiv) in tetrahydrofuran (5 mL) was added dropwise 1M diisobutylaluminium hydride (14.9 mL, 14.9 mmol, 2 equiv) under nitrogen atmosphere. The mixture was stirred at −78° C. for 3 h. The reaction was quenched with saturated aqueous ammonium chloride (100 mL) and the mixture was extracted with ethyl acetate (2×100 mL). The combined organic extracts were washed with brine (3×100 mL), dried with sodium sulfate, filtered, and concentrated. The residue was purified by silica gel column chromatography eluting with 50% ethyl acetate in petroleum ether to afford (5-bromo-2-(dimethylamino)-1-((2-(trimethylsilyl)ethoxy)methyl)-1H-benzo[d]imidazol-7-yl)methanol (1.7 g, 52%) as a yellow solid. MS (ESI) calcd. for C₁₆H₂₆BrN₃O₂Si: 399.10 m/z, found 400.05 [M+H]⁺.

### Step 4: Synthesis of 5-bromo-2-(dimethylamino)-1-((2-(trimethylsilyl)ethoxy)methyl)-1H-benzo[d]imidazole-7-carbaldehyde

To a mixture of (5-bromo-2-(dimethylamino)-1-((2-(trimethylsilyl)ethoxy)methyl)-1H-benzo[d]imidazol-7-yl)methanol (1.6 g, 4 mmol, 1 equiv) in dichloromethane (40 mL) was added Dess-Martin Periodinane (2.03 g, 4.80 mmol, 1.2 equiv) and the mixture was stirred at room temperature for 2 h. Water (200 mL) was added and the mixture was extracted with ethyl acetate (200 mL×3). The combined organic phases were washed with brine (30 mL×3), dried with sodium sulfate, filtered and concentrated. The residue was purified by silica gel column chromatography eluting with 50% ethyl acetate in petroleum ether to afford 5-bromo-2-(dimethylamino)-1-((2-(trimethylsilyl)ethoxy)methyl)-1H-benzo[d]imidazole-7-carbaldehyde (1.5 g, 92%) as a yellow solid. MS (ESI) calcd. for C₁₆H₂₄BrN₃O₂Si: 397.08 m/z, found 398.00 [M+H]⁺.

### Step 5: Synthesis of 5-bromo-7-(1,3-dioxolan-2-yl)-N,N-dimethyl-1-{[2-(trimethylsilyl)ethoxy]methyl}-1,3-benzodiazol-2-amine

A mixture of 5-bromo-2-(dimethylamino)-1-((2-(trimethylsilyl)ethoxy)methyl)-1H-benzo[d]imidazole-7-carbaldehyde (1.5 g, 3.8 mmol, 1 equiv), ethylene glycol (1.05 mL, 18.8 mmol, 5 equiv), p-toluenesulfonic acid (0.060 g, 0.38 mmol, 0.1 equiv) and triethyl orthoformate (1.88 mL, 11.3 mmol, 3 equiv) in toluene (75 mL) was stirred at 110° C. overnight. The mixture was cooled to room temperature and taken up into ethyl acetate (50 mL) and water (50 mL). After separation of phases, the aqueous phase was extracted with ethyl acetate (100 mL×2). The combined organic phases were washed with brine (100 mL×3), dried with sodium sulfate, filtered and concentrated. The residue was purified by silica gel column chromatography eluting with 50% ethyl acetate in petroleum ether to afford 5-bromo-7-(1,3-dioxolan-2-yl)-N,N-dimethyl-1-{[2-(trimethylsilyl)ethoxy]methyl}-1,3-benzodiazol-2-amine (800 mg, 45%) as a yellow solid. MS (ESI) calcd. for C₁₈H₂₈BrN₃O₃Si: 441.11 m/z, found 442.15 [M+H]⁺.

### Step 6: Synthesis of 2-(dimethylamino)-7-(1,3-dioxolan-2-yl)-1-((2-(trimethylsilyl)ethoxy)methyl)-1H-benzo[d]imidazole-5-carboxylic acid (Intermediate 175-1)

To a cooled (−78° C.) solution of 5-bromo-7-(1,3-dioxolan-2-yl)-N,N-dimethyl-1-{[2-(trimethylsilyl)ethoxy]methyl}-1,3-benzodiazol-2-amine (200 mg, 0.452 mmol, 1 equiv) in tetrahydrofuran (5 mL) was added dropwise n-butyllithium solution (2 M in hexane, 0.27 mL, 0.54 mmol, 1.2 equiv) under N₂ atmosphere. The reaction mixture was stirred at −78° C. for 1 h under nitrogen then for 2 h under carbon dioxide. The reaction was quenched with saturated aqueous ammonium chloride (1 mL) and the mixture was extracted with ethyl acetate 3 times. The combined organic layers were washed with brine, dried over anhydrous sodium sulfate, filtered and concentrated in vacuo to yield crude 2-(dimethylamino)-7-(1,3-dioxolan-2-yl)-1-((2-(trimethylsilyl)ethoxy)methyl)-1H-benzo[d]imidazole-5-carboxylic acid (Intermediate 175-1) (200 mg, 75%) as a yellow solid, which was used in subsequent transformations without further purification. MS (ESI) calcd. for C₁₉H₂₉N₃O₅Si: 407.19 m/z, found 408.10 [M+H]⁺.

### Example 176: (S)—N-(5-(2-(2-aminopyridin-3-yl)-5-phenyl-3H-imidazo[4,5-b]pyridin-3-yl)-2,3-dihydro-1H-inden-1-yl)-6-chloro-4-formyl-5-hydroxypicolinamide

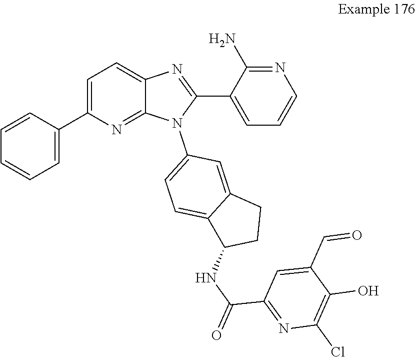 [SMILES: Nc1ncccc1-c1nc2ccc(-c3ccccc3)nc2n1-c1ccc2c(c1)CC[C@@H]2NC(=O)c1cc(C=O)c(O)c(Cl)n1]

Example 176 was prepared in a manner analogous to Example 168 (starting from Step 3) using Intermediate 1-3 in place of Intermediate 33-1 and 7-chlorofuro[2,3-c]pyridine-5-carboxylic acid in place of 7-(difluoromethoxy)benzofuran-5-carboxylic acid. MS (ESI) calculated for C₃₃H₂₄ClN₇O₃: 601.16, found 602.31 [M+H]⁺. ¹H NMR (400 MHz, Acetic acid-d₄) δ (ppm): 10.95 (s, 1H), 8.93 (d, J=8.4 Hz, 1H), 8.88 (s, 1H), 8.74-8.60 (m, 5H), 8.37 (dd, J=7.5, 1.3 Hz, 1H), 8.15-7.94 (m, 7H), 7.43 (dd, J=7.4, 6.1 Hz, 1H), 6.29 (t, J=8.3 Hz, 1H), 3.68 (ddd, J=11.4, 9.5, 2.8 Hz, 1H), 3.60-3.47 (m, 1H), 3.12-3.07 (m, 1H), 2.87 (dq, J=12.3, 8.8 Hz, 1H).

### Example 177: 2-[(1S)-5-[2-(2-aminopyridin-3-yl)-5-(pyrazol-1-yl)imidazo[4,5-b]pyridin-3-yl]-2,3-dihydro-1H-inden-1-yl]-6-hydroxy-1-oxo-3,4-dihydroisoquinoline-7-carbaldehyde

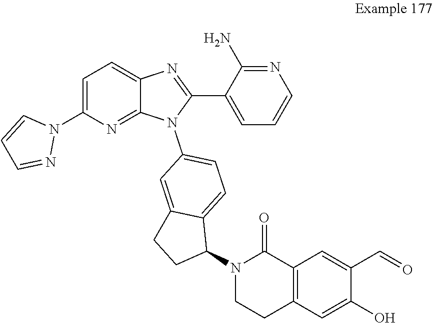 [SMILES: Nc1ncccc1-c1nc2ccc(-n3cccn3)nc2n1-c1ccc2c(c1)CC[C@@H]2N1CCc2cc(O)c(C=O)cc2C1=O]

### Synthetic Route:

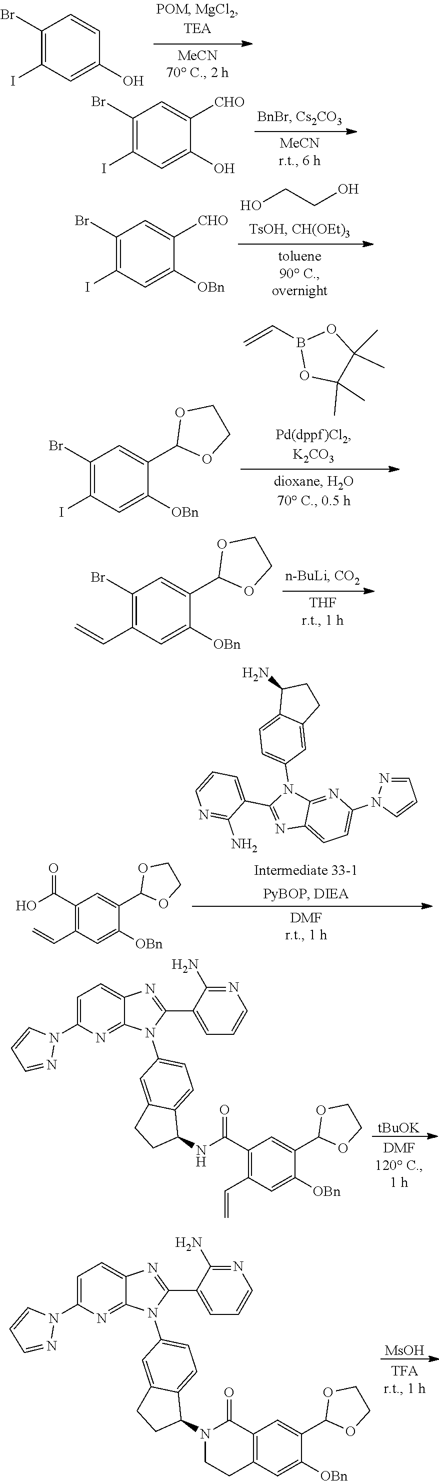

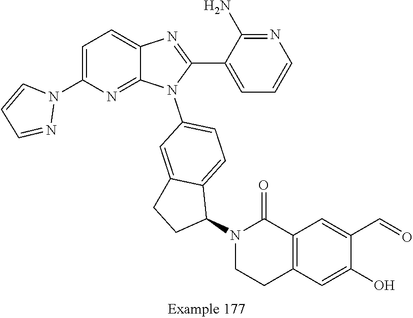 [SMILES: Nc1ncccc1-c1nc2ccc(-n3cccn3)nc2n1-c1ccc2c(c1)CC[C@@H]2N1CCc2cc(O)c(C=O)cc2C1=O]

### Step 1: Synthesis of 5-bromo-2-hydroxy-4-iodobenzaldehyde

To a solution of 4-bromo-3-iodophenol (3.00 g, 10.0 mmol, 1 equiv) in tetrahydrofuran (40 mL) were added paraformaldehyde (4.52 g, 50.2 mmol, 5 equiv), magnesium (II) chloride (1.43 g, 15.1 mmol, 1.5 equiv) and triethylamine (2.54 g, 25.1 mmol, 2.5 equiv). The obtained suspension was stirred at room temperature for 10 min then at 70° C. for 2 h. The mixture was cooled to 0° C. and quenched by the addition of 2M hydrochloric acid. The resulting mixture was extracted with ethyl acetate (20 mL×3). The combined organic layers were washed with water (20 mL×3), dried over anhydrous sodium sulfate, filtered and concentrated under reduced pressure. The residue was purified by silica gel column chromatography using a 0-30% gradient of ethyl acetate in petroleum ether to afford 5-bromo-2-hydroxy-4-iodobenzaldehyde (1 g, 30%) as a light-yellow solid. MS (ESI) calcd. for C₇H₄BrIO₂: 325.84 m/z, found 326.95 [M+H]⁺.

### Step 2: Synthesis of 2-(benzyloxy)-5-bromo-4-iodobenzaldehyde

To a solution of 5-bromo-2-hydroxy-4-iodobenzaldehyde (400 mg, 1.22 mmol, 1 equiv) in acetonitrile (10 mL) were added benzyl bromide (314 mg, 1.84 mmol, 1.4 equiv) and cesium carbonate (797 mg, 2.45 mmol, 2 equiv). The resulting mixture was stirred at room temperature for 6 h. The reaction was quenched by the addition of water at 0° C. The resulting mixture was extracted with ethyl acetate (20 mL×3). The combined organic layers were washed with water (10 mL×3), dried over anhydrous sodium sulfate, filtered and concentrated under reduced pressure. The residue was triturated with petroleum ether to afford 2-(benzyloxy)-5-bromo-4-iodobenzaldehyde (300 mg, 59%) as a white solid. MS (ESI) calcd. for C₁₄H₁₀BrIO₂: 415.89 m/z, found 416.90 [M+H]⁺.

### Step 3: Synthesis of 2-[2-(benzyloxy)-5-bromo-4-iodophenyl]-1,3-dioxolane

To a solution of 2-(benzyloxy)-5-bromo-4-iodobenzaldehyde (300 mg, 0.719 mmol, 1 equiv) in toluene (5 mL) were added ethylene glycol (223 mg, 3.60 mmol, 5 equiv), triethyl orthoformate (320 mg, 2.16 mmol, 3 equiv) and p-toluenesulfonic acid (12 mg, 0.072 mmol, 0.1 equiv). The obtained solution was stirred at room temperature for 10 min then at 90° C. overnight. The reaction was cooled to room temperature and quenched by the addition of saturated aqueous sodium bicarbonate. The resulting mixture was extracted with ethyl acetate (10 mL×3). The combined organic layers were washed with water (10 mL×3), dried over anhydrous sodium sulfate, filtered and concentrated under reduced pressure. The residue was purified by silica gel column chromatography using a 0-17% gradient of ethyl acetate in petroleum ether to afford 2-[2-(benzyloxy)-5-bromo-4-iodophenyl]-1,3-dioxolane (300 mg, 90% yield) as a white solid. MS (ESI) calcd. for C₁₆H₁₄BrIO₃: 459.92 m/z, found 460.95 [M+H]⁺.

### Step 4: Synthesis of 2-[2-(benzyloxy)-5-bromo-4-ethenylphenyl]-1,3-dioxolane

To a solution of 2-[2-(benzyloxy)-5-bromo-4-iodophenyl]-1,3-dioxolane (300 mg, 1.08 mmol, 1 equiv) in 1,4-dioxane (3.2 mL) and water (0.8 mL) were added 2-ethenyl-4,4,5,5-tetramethyl-1,3,2-dioxaborolane (167 mg, 1.08 mmol, 1 equiv), [1,1′-bis(diphenylphosphino)ferrocene]dichloropalladium(II) (79 mg, 0.108 mmol, 0.1 equiv) and potassium carbonate (300 mg, 2.17 mmol, 2 equiv) and the resulting mixture was stirred for 0.5 h at 70° C. under nitrogen atmosphere. The reaction cooled to 0° C. and quenched by the addition of saturated aqueous ammonium chloride (5 mL). The resulting mixture was extracted with ethyl acetate (20 mL×3). The combined organic layers were washed with water (10 mL×3), dried over anhydrous sodium sulfate, filtered and concentrated under reduced pressure. The residue was purified by silica gel column chromatography using a 0-17% gradient of ethyl acetate in petroleum ether to afford 2-[2-(benzyloxy)-5-bromo-4-ethenylphenyl]-1,3-dioxolane (200 mg, 51% yield) as a light-yellow solid. MS (ESI) calcd. for C₁₈H₁₇BrO₃: 360.04 m/z, found 361.05 [M+H]⁺.

### Step 5: Synthesis of 4-(benzyloxy)-5-(1,3-dioxolan-2-yl)-2-ethenylbenzoic acid

To a cooled (−78° C.) solution of 2-[2-(benzyloxy)-5-bromo-4-ethenylphenyl]-1,3-dioxolane (200 mg, 0.554 mmol, 1 equiv) in tetrahydrofuran (10 mL) was added n-butyllithium (2.5 M in hexane, 1.38 mL, 3.45 mmol, 6 equiv) and the resulting mixture was stirred for 1 h under nitrogen atmosphere then for an additional 1 h under carbon dioxide. The reaction was quenched by the addition of saturated aqueous ammonium chloride (10 mL). The resulting mixture was concentrated under reduced pressure and the residue was purified by reverse-phase flash column chromatography on C18 silica gel using a 10-50% gradient of acetonitrile in water (+0.05% ammonium bicarbonate) to afford 4-(benzyloxy)-5-(1,3-dioxolan-2-yl)-2-ethenylbenzoic acid (100 mg, 55% yield) as a white solid. MS (ESI) calcd. for C₁₉H₁₈O₅: 326.12 m/z, found 327.20 [M+H]⁺.

### Step 6: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-ethenylbenzamide

To a suspension of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (100 mg, 0.245 mmol, 1 equiv) in N,N-dimethylformamide (3 mL) were added 4-(benzyloxy)-5-(1,3-dioxolan-2-yl)-2-ethenylbenzoic acid (80 mg, 0.25 mmol, 1 equiv), PyBOP (153 mg, 0.294 mmol, 1.2 equiv) and N,N-diisopropylethylamine (95 mg, 0.735 mmol, 3 equiv) and the mixture was stirred at room temperature for 1 h. The residue was purified by reverse-phase flash column chromatography on C18 silica gel using a 10-80% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-ethenylbenzamide (90 mg, 51% yield) as a light-yellow solid. MS (ESI) calcd. for C₄₂H₃₆N₈O₄: 716.29 m/z, found 717.30 [M+H]⁺.

### Step 7: Synthesis of 2-[(1S)-5-[2-(2-aminopyridin-3-yl)-5-(pyrazol-1-yl)imidazo[4,5-b]pyridin-3-yl]-2,3-dihydro-1H-inden-1-yl]-6-(benzyloxy)-7-(1,3-dioxolan-2-yl)-3,4-dihydroisoquinolin-1-one

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-ethenylbenzamide (90 mg, 0.13 mmol, 1 equiv) in N,N-dimethylformamide (2 mL) was added potassium tert-butoxide (1M, 0.25 mL, 0.25 mmol, 2 equiv) and the resulting mixture was stirred for 1 h at 120° C. under a nitrogen atmosphere. The reaction was quenched by the addition of saturated aqueous ammonium chloride (3 mL) and extracted with ethyl acetate (3×20 mL). The combined organic layers were washed with brine (3×10 mL), dried over anhydrous sodium sulfate, filtered and concentrated under reduced pressure to afford 2-[(1S)-5-[2-(2-aminopyridin-3-yl)-5-(pyrazol-1-yl)imidazo[4,5-b]pyridin-3-yl]-2,3-dihydro-1H-inden-1-yl]-6-(benzyloxy)-7-(1,3-dioxolan-2-yl)-3,4-dihydroisoquinolin-1-one (60 mg, 67% yield) as a light-yellow solid. MS (ESI) calcd. for C₄₂H₃₆N₈O₄: 716.29 m/z, found 717.30 [M+H]⁺.

### Step 8: Synthesis of 2-[(1S)-5-[2-(2-aminopyridin-3-yl)-5-(pyrazol-1-yl)imidazo[4,5-b]pyridin-3-yl]-2,3-dihydro-1H-inden-1-yl]-6-hydroxy-1-oxo-3,4-dihydroisoquinoline-7-carbaldehyde (Example 177)

To a cooled (0° C.) solution of 2-[(1S)-5-[2-(2-aminopyridin-3-yl)-5-(pyrazol-1-yl)imidazo[4,5-b]pyridin-3-yl]-2,3-dihydro-1H-inden-1-yl]-6-(benzyloxy)-7-(1,3-dioxolan-2-yl)-3,4-dihydroisoquinolin-1-one (50 mg, 0.070 mmol, 1 equiv) in 2,2,2-trifluoroacetic acid (3 mL) was added dropwise methanesulfonic acid (0.6 mL). The reaction mixture was stirred at room temperature for 1 h. The mixture was concentrated under reduced pressure and was purified by preparative HPLC on a XSelect CSH Prep C18 OBD Column using a 47-69% gradient of acetonitrile in water (+0.1% formic acid) to afford 2-[(1S)-5-[2-(2-aminopyridin-3-yl)-5-(pyrazol-1-yl)imidazo[4,5-b]pyridin-3-yl]-2,3-dihydro-1H-inden-1-yl]-6-hydroxy-1-oxo-3,4-dihydroisoquinoline-7-carbaldehyde (Example 177) (4.9 mg, 12% yield) as a yellow solid. MS (ESI) calcd. for C₃₃H₂₆N₈O₃: 582.21 m/z, found 583.25 [M+H]⁺. ¹H NMR (400 MHz, DMSO-d₆) δ (ppm): 10.23 (s, 1H), 8.67 (s, 1H), 8.28-8.53 (m, 4H), 7.84-8.06 (m, 3H), 7.57-7.67 (m, 1H), 7.13-7.24 (m, 1H), 6.99-7.03 (m, 1H), 6.87 (s, 1H), 6.56-6.68 (m, 1H), 6.15-6.26 (m, 1H), 3.29-3.32 (m, 1H), 3.03-3.11 (m, 2H), 2.89-2.97 (m, 3H), 2.41-2.44 (m, 1H), 1.99-2.07 (m, 1H).

### Example 178: N-[(1S)-5-[2-(2-aminopyridin-3-yl)-5-(4-methyl-1,3-oxazol-2-yl)imidazo[4,5-b]pyridin-3-yl]-2,3-dihydro-1H-inden-1-yl]-3-formyl-4-hydroxybenzamide

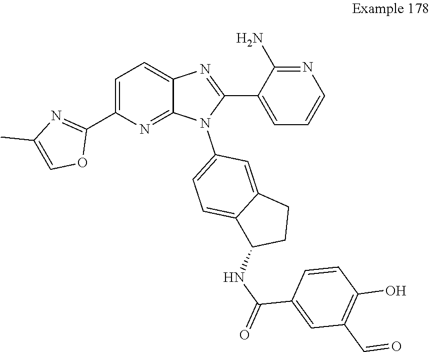 [SMILES: Cc1coc(-c2ccc3nc(-c4cccnc4N)n(-c4ccc5c(c4)CC[C@@H]5NC(=O)c4ccc(O)c(C=O)c4)c3n2)n1]

Example 178 was prepared in a manner analogous to Example 121 using Intermediate 178-1 in place of 2-(tributylstannyl)-1,3-oxazole. MS (ESI) calcd. for C₃₂H₂₅N₇O₄: 571.20 m/z, found 572.25 [M+H]⁺. ¹H NMR (300 MHz, DMSO-d₆) δ (ppm): 10.30 (s, 1H), 8.98-9.01 (m, 1H), 8.30-8.33 (m, 2H), 8.02-8.15 (m, 2H), 8.00-8.01 (m, 1H), 7.92-7.93 (m, 1H), 7.38-7.47 (m, 2H), 7.28-7.32 (m, 2H), 7.05-7.08 (m, 1H), 6.43-6.47 (m, 1H), 5.60-5.65 (m, 1H), 2.87-3.01 (m, 1H), 2.73-2.74 (m, 1H), 2.52-2.53 (m, 1H), 2.17-2.18 (m, 3H), 2.01-2.11 (m, 1H).

### Intermediate 178-1: 4-methyl-2-(tributylstannyl)oxazole

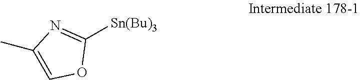

### Synthetic Route:

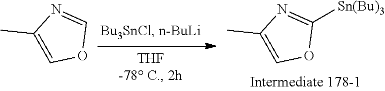

### Step 1: Synthesis of 4-methyl-2-(tributylstannyl)-1,3-oxazole (Intermediate 178-1)

To a cooled (−78° C.) solution of 4-methyloxazole (2.00 g, 24.1 mmol, 1 equiv) in tetrahydrofuran (80 mL) was added dropwise n-butyllithium (2.5 M in hexane, 9.63 mL, 24 mmol, 1 equiv) under nitrogen atmosphere. The resulting mixture was stirred at −78° C. for 30 mins then tributyl(chloro)stannane (7.84 g, 24.1 mmol, 1 equiv) was added and the mixture was stirred for another 1 h. The reaction was quenched with water (100 mL) and the resulting mixture was extracted with ethyl acetate (3×150 mL). The combined organic layers were washed with brine (3×150 mL), dried over anhydrous sodium sulfate, filtered and concentrated under reduced pressure to afford 4-methyl-2-(tributylstannyl)-1,3-oxazole (6.8 g, 76%) as a yellow oil, which was used in subsequent transformations without further purification. MS (ESI) calcd. for C₁₆H₃₁NOSn: 373.14 m/z, found 374.25 [M+H]⁺.

### Example 179: N-[(1S)-5-[2-(2-aminopyridin-3-yl)-5-(3-methyl-1,2,4-oxadiazol-5-yl)imidazo[4,5-b]pyridin-3-yl]-2,3-dihydro-1H-inden-1-yl]-3-formyl-4-hydroxybenzamide

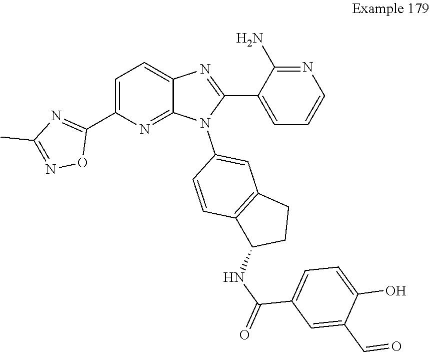 [SMILES: Cc1noc(-c2ccc3nc(-c4cccnc4N)n(-c4ccc5c(c4)CC[C@@H]5NC(=O)c4ccc(O)c(C=O)c4)c3n2)n1]

Example 179 was prepared in a manner analogous to Example 7 using Intermediate 179-1 in place of Intermediate 1-2. MS (ESI) calcd. for C₃₁H₂₄N₈O₄: 572.19 m/z, found 573.25 [M+H]⁺. ¹H NMR (400 MHz, DMSO-d₆) δ (ppm): 10.29 (s, 1H), 8.99-9.01 (m, 1H), 8.40-8.42 (m, 1H), 8.30-8.31 (m, 1H), 8.24-8.27 (m, 1H), 8.08-8.11 (m, 1H), 8.03-8.04 (m, 1H), 7.37-7.41 (m, 2H), 7.31-7.34 (m, 2H), 7.06-7.08 (m, 1H), 6.46-6.50 (m, 1H), 5.62-5.66 (m, 1H), 3.01-3.07 (m, 1H), 2.89-2.90 (m, 1H), 2.52-2.53 (m, 1H), 2.41-2.42 (m, 3H), 2.09-2.12 (m, 1H).

### Intermediate 179-1: (S)-3-(3-(1-amino-2,3-dihydro-1H-inden-5-yl)-5-(3-methyl-1,2,4-oxadiazol-5-yl)-3H-imidazo[4,5-b]pyridin-2-yl)pyridin-2-amine

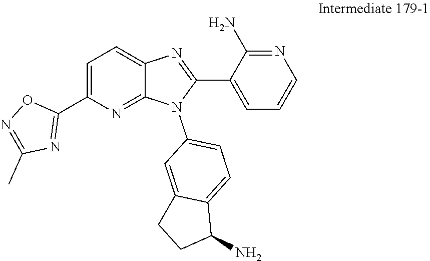 [SMILES: Cc1noc(-c2ccc3nc(-c4cccnc4N)n(-c4ccc5c(c4)CC[C@@H]5N)c3n2)n1]

### Synthetic Route:

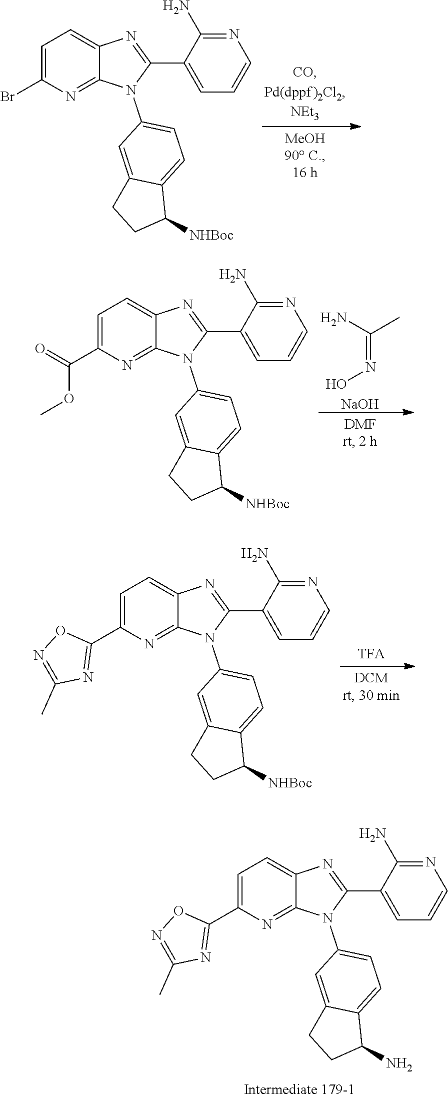

### Step 1: Synthesis of methyl 2-(2-aminopyridin-3-yl)-3-[(1S)-1-[(tert-butoxycarbonyl)amino]-2,3-dihydro-1H-inden-5-yl]imidazo[4,5-b]pyridine-5-carboxylate

A solution of tert-butyl N-[(1S)-5-[2-(2-aminopyridin-3-yl)-5-bromoimidazo[4,5-b]pyridin-3-yl]-2,3-dihydro-1H-inden-1-yl]carbamate (Intermediate 173-1) (2.00 g, 3.84 mmol, 1 equiv), triethylamine (1.94 g, 19.2 mmol, 5 equiv) and [1,1′-bis(diphenylphosphino)ferrocene]dichloropalladium(II)-dichloromethane complex (0.31 g, 0.38 mmol, 0.1 equiv) in N,N-dimethylacetamide (100 mL) and methanol (40 mL) was stirred at 90° C. for 16 h under carbon monoxide (30 atm). The reaction mixture was cooled to room temperature and water (1000 mL) was added. The precipitated solids were collected by filtration and washed with water (3×10 mL). The solids were purified by silica gel column chromatography using a 0-50% gradient of ethyl acetate in petroleum ether to afford methyl 2-(2-aminopyridin-3-yl)-3-[(1S)-1-[(tert-butoxycarbonyl)amino]-2,3-dihydro-1H-inden-5-yl]imidazo[4,5-b]pyridine-5-carboxylate (1.74 g, 77%) as a brown/yellow solid. MS (ESI) calcd. for C₂₇H₂₈N₆O₄: 500.22 m/z, found 501.25 [M+H]⁺.

### Step 2: Synthesis of tert-butyl N-[(1S)-5-[2-(2-aminopyridin-3-yl)-5-(3-methyl-1,2,4-oxadiazol-5-yl)imidazo[4,5-b]pyridin-3-yl]-2,3-dihydro-1H-inden-1-yl]carbamate

A solution of methyl 2-(2-aminopyridin-3-yl)-3-[(1S)-1-[(tert-butoxycarbonyl)amino]-2,3-dihydro-1H-inden-5-yl]imidazo[4,5-b]pyridine-5-carboxylate (500 mg, 0.999 mmol, 1 equiv) and (Z)—N′-hydroxyacetimidamide (89 mg, 1.2 mmol, 1.2 equiv) and sodium hydroxide (80 mg, 2.0 mmol, 2 equiv) in N,N-dimethylformamide (10 mL) was stirred at 25° C. for 2 h under nitrogen atmosphere. Water was added and the resulting mixture was extracted with ethyl acetate (100 mL×2). The combined organic layers were washed with brine, dried over anhydrous sodium sulfate, filtered and concentrated under reduced pressure. The crude was purified by the silica gel column chromatography using a 0-100% gradient of ethyl acetate in petroleum ether giving tert-butyl N-[(1S)-5-[2-(2-aminopyridin-3-yl)-5-(3-methyl-1,2,4-oxadiazol-5-yl)imidazo[4,5-b]pyridin-3-yl]-2,3-dihydro-1H-inden-1-yl]carbamate (458 mg, 81%) as a yellow solid. MS (ESI) calcd. for C₂₈H₂₈N₈O₃: 524.23 m/z, found 525.30 [M+H]⁺.

### Step 3: Synthesis of 3-{3-[(1S)-1-amino-2,3-dihydro-1H-inden-5-yl]-5-(3-methyl-1,2,4-oxadiazol-5-yl)imidazo[4,5-b]pyridin-2-yl}pyridin-2-amine (Intermediate 179-1)

To a solution of tert-butyl N-[(1S)-5-[2-(2-aminopyridin-3-yl)-5-(3-methyl-1,2,4-oxadiazol-5-yl)imidazo[4,5-b]pyridin-3-yl]-2,3-dihydro-1H-inden-1-yl]carbamate (430 mg, 0.820 mmol) dichloromethane (9 mL) was added 2,2,2-trifluoroacetic acid (3 mL) and the resulting mixture was stirred at 25° C. for 0.5 h. The mixture was brought to pH 7-8 with saturated aqueous sodium bicarbonate. The resulting mixture was extracted with 3:1 chloroform/isopropanol (100 mL×3). The combined organic layers were washed with brine, dried over anhydrous sodium sulfate, filtered and concentrated under reduced pressure to afford 3-{3-[(1S)-1-amino-2,3-dihydro-1H-inden-5-yl]-5-(3-methyl-1,2,4-oxadiazol-5-yl)imidazo[4,5-b]pyridin-2-yl}pyridin-2-amine (Intermediate 179-1) (340 mg, 91%) as a brown solid. MS (ESI) calcd. for C₂₃H₂₀N₈O: 424.18 m/z, found 425.20 [M+H]⁺.

### Example 180: N-[(1S)-5-[2-(2-aminopyridin-3-yl)-7-methyl-5-(1,3-thiazol-2-yl)imidazo[4,5-b]pyridin-3-yl]-2,3-dihydro-1H-inden-1-yl]-3-formyl-4-hydroxybenzamide

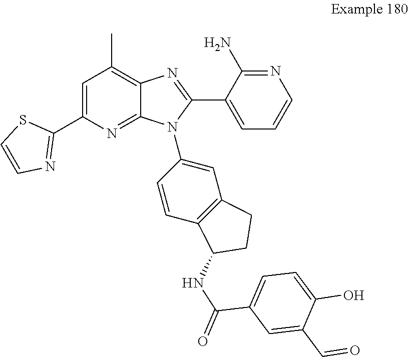 [SMILES: Cc1cc(-c2nccs2)nc2c1nc(-c1cccnc1N)n2-c1ccc2c(c1)CC[C@@H]2NC(=O)c1ccc(O)c(C=O)c1]

Example 180 was prepared in a manner analogous to Example 12 using Intermediate 180-1 in place of Intermediate 1-3 and Intermediate 57-2 in place of Intermediate 12-1. MS (ESI) calcd. for C₃₂H₂₅N₇O₃S: 587.17 m/z, found 588.25 [M+H]⁺. ¹H NMR (300 MHz, DMSO-d₆) δ (ppm): 10.31 (s, 1H), 8.98-9.01 (m, 1H), 8.31-8.32 (m, 1H), 8.01-8.14 (m, 3H), 7.93-7.94 (m, 1H), 7.75-7.76 (m, 1H), 7.26-7.38 (m, 4H), 7.07-7.10 (m, 1H), 6.46-6.50 (m, 1H), 5.51-5.65 (m, 1H), 2.81-3.12 (m, 2H), 2.66-2.74 (m, 3H), 2.51-2.52 (m, 1H), 2.03-2.20 (m, 1H).

### Intermediate 180-1: (S)-3-(3-(1-amino-2,3-dihydro-1H-inden-5-yl)-7-methyl-5-(thiazol-2-yl)-3H-imidazo[4,5-b]pyridin-2-yl)pyridin-2-amine

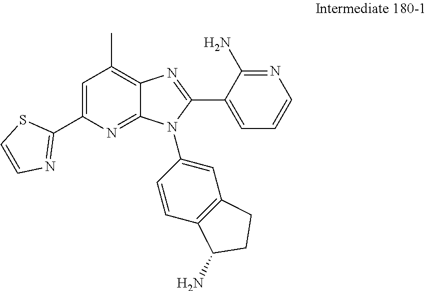 [SMILES: Cc1cc(-c2nccs2)nc2c1nc(-c1cccnc1N)n2-c1ccc2c(c1)CC[C@@H]2N]

Intermediate 180-1 was prepared in a manner analogous to Intermediate 57-1 using Intermediate 131-1 in place of Intermediate 79-1. MS (ESI) calcd. for C₂₄H₂₁N₇S: 439.16 m/z, found 440.25 [M+H]⁺.

### Example 181: (S)—N-(5-(2-(2-aminopyridin-3-yl)-5-(3-fluoro-1H-pyrazol-1-yl)-7-methyl-3H-imidazo[4,5-b]pyridin-3-yl)-2,3-dihydro-1H-inden-1-yl)-3-formyl-4-hydroxybenzamide

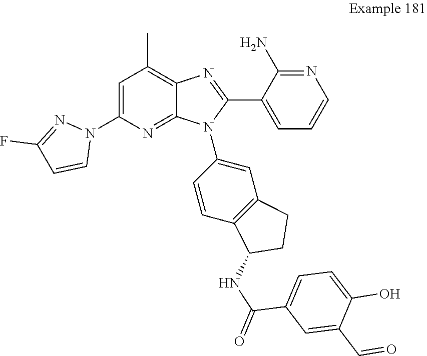 [SMILES: Cc1cc(-n2ccc(F)n2)nc2c1nc(-c1cccnc1N)n2-c1ccc2c(c1)CC[C@@H]2NC(=O)c1ccc(O)c(C=O)c1]

Example 181 was prepared in a manner analogous to Example 12 using Intermediate 181-1 in place of Intermediate 1-3 and Intermediate 57-2 in place of Intermediate 12-1. MS (ESI) calcd. for C₃₂H₂₆N₈O₃: 588.20 m/z, found 589.15 [M+H]⁺. ¹H NMR (400 MHz, DMSO-d₆) δ (ppm): 10.32 (s, 1H), 8.30-8.31 (m, 2H), 8.07-8.11 (m, 2H), 7.70-7.76 (m, 2H), 7.43-7.44 (m, 1H), 7.30-7.36 (m, 2H), 7.07-7.09 (m, 1H), 6.80-6.81 (m, 1H), 6.34-6.36 (m, 1H), 5.59-5.63 (m, 1H), 3.02-3.10 (m, 1H), 2.92-2.94 (m, 1H), 2.67-2.75 (m, 3H), 2.50-2.51 (m, 1H), 2.07-2.10 (m, 1H). ¹⁹F NMR (376 MHz, DMSO-d₆) δ (ppm): −127.29. (2,2,2-trifluoroacetic acid salt).

### Intermediate 181-1: (S)-3-(3-(1-amino-2,3-dihydro-1H-inden-5-yl)-5-(3-fluoro-1H-pyrazol-1-yl)-7-methyl-3H-imidazo[4,5-b]pyridin-2-yl)pyridin-2-amine

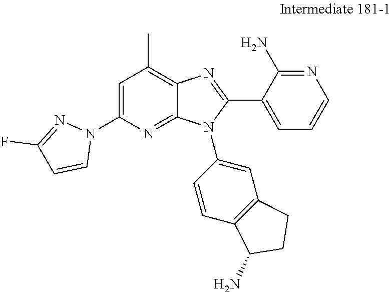 [SMILES: Cc1cc(-n2ccc(F)n2)nc2c1nc(-c1cccnc1N)n2-c1ccc2c(c1)CC[C@@H]2N]

Intermediate 181-1 was prepared in a manner analogous to Intermediate 33-1 using Intermediate 131-1 in place of Intermediate 18-2 and 3-fluoropyrazole in place of pyrazole. MS (ESI) calcd. for C₂₄H₂₁FN₈: 440.19 m/z, found 441.15 [M+H]⁺.

### Example 182: N-[(1S)-5-[2-(2-aminopyridin-3-yl)-5-(pyrazol-1-yl)imidazo[4,5-b]pyridin-3-yl]-2,3-dihydro-1H-inden-1-yl]-3-formyl-4-hydroxy-5-methoxybenzamide

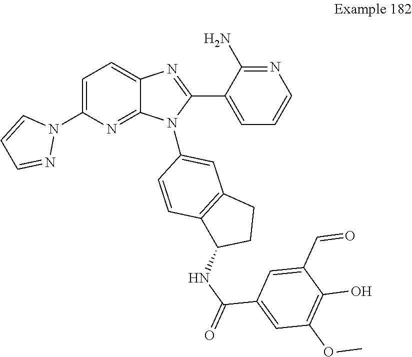 [SMILES: COc1cc(C(=O)N[C@H]2CCc3cc(-n4c(-c5cccnc5N)nc5ccc(-n6cccn6)nc54)ccc32)cc(C=O)c1O]

Example 182 was prepared in a manner analogous to Example 12 using Intermediate 33-1 in place of Intermediate 1-3 and Intermediate 36-1 in place of Intermediate 12-1. MS (ESI) calcd. for C₃₂H₂₆N₈O₄: 586.21 m/z, found 587.25 [M+H]⁺. ¹H NMR (300 MHz, DMSO-d₆) δ (ppm): 10.31 (s, 1H), 8.36-8.37 (m, 2H), 8.02-8.03 (m, 1H), 7.94-7.96 (m, 2H), 7.81-7.88 (m, 1H), 7.60-7.62 (m, 1H), 7.35-7.38 (m, 1H), 7.30-7.33 (m, 1H), 7.25-7.29 (m, 2H), 6.55-6.56 (m, 1H), 6.48-6.55 (m, 1H), 5.62-5.65 (m, 1H), 3.72-3.85 (m, 3H), 3.00-3.04 (m, 1H), 2.88-2.92 (m, 1H), 2.48-2.52 (m, 1H), 2.08-2.13 (m, 1H).

### Example 183: (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-3-(formyl-d)-4-hydroxybenzamide

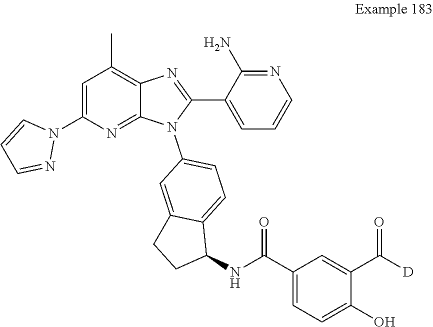 [SMILES: [2H]C(=O)c1cc(C(=O)N[C@H]2CCc3cc(-n4c(-c5cccnc5N)nc5c(C)cc(-n6cccn6)nc54)ccc32)ccc1O]

### Synthetic Route:

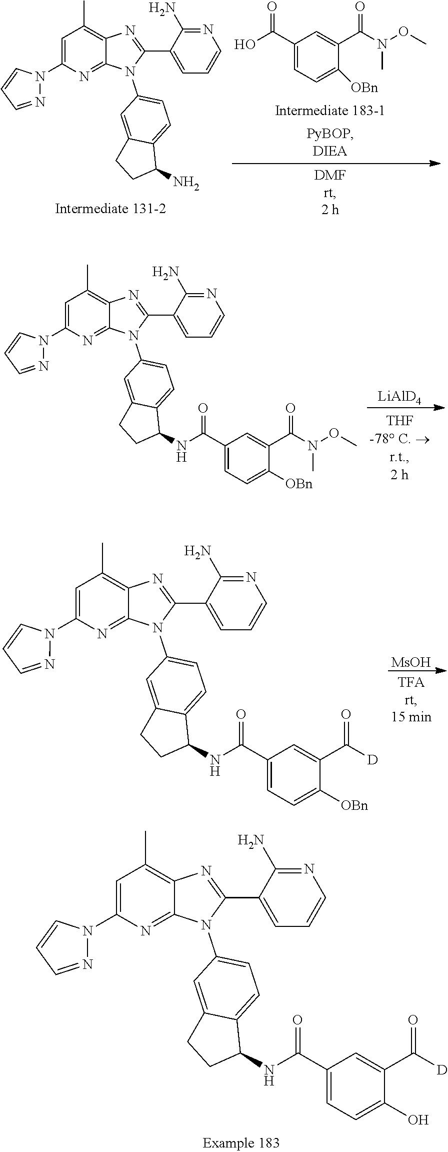

### Step 1: N3-[(1S)-5-[2-(2-aminopyridin-3-yl)-7-methyl-5-(pyrazol-1-yl)imidazo[4,5-b]pyridin-3-yl]-2,3-dihydro-1H-inden-1-yl]-6-(benzyloxy)-N1-methoxy-N1-methylbenzene-1,3-dicarboxamide

To a stirred solution of 3-{3-[(1S)-1-amino-2,3-dihydro-1H-inden-5-yl]-7-methyl-5-(pyrazol-1-yl)imidazo[4,5-b]pyridin-2-yl}pyridin-2-amine (Intermediate 131-2) (161 mg, 0.381 mmol, 1.2 equiv) in N,N-dimethylformamide (5 mL) was added N,N-diisopropylethylamine (123 mg, 0.951 mmol, 3 equiv), PyBOP (198 mg, 0.381 mmol, 1.2 equiv) and 4-(benzyloxy)-3-[methoxy(methyl)carbamoyl]benzoic acid (Intermediate 183-1) (100 mg, 0.317 mmol, 1 equiv) and the solution was stirred at room temperature for 2 h then purified by reverse-phase flash column chromatography on C18 silica gel using a 10-60% gradient of acetonitrile in water (+0.05% ammonium bicarbonate) to afford N3-[(1S)-5-[2-(2-aminopyridin-3-yl)-7-methyl-5-(pyrazol-1-yl)imidazo[4,5-b]pyridin-3-yl]-2,3-dihydro-1H-inden-1-yl]-6-(benzyloxy)-N1-methoxy-N1-methylbenzene-1,3-dicarboxamide (60 mg, 26%) as a white solid. MS (ESI) calcd. for C₄₁H₃₇N₉O₄: 719.30 m/z, found 720.35 [M+H]⁺.

### Step 2: (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-4-(benzyloxy)-3-(formyl-d)benzamide

To a cooled (−78° C.) solution of N3-[(1S)-5-[2-(2-aminopyridin-3-yl)-7-methyl-5-(pyrazol-1-yl)imidazo[4,5-b]pyridin-3-yl]-2,3-dihydro-1H-inden-1-yl]-6-(benzyloxy)-N1-methoxy-N1-methylbenzene-1,3-dicarboxamide (60 mg, 0.083 mmol, 1 equiv) in tetrahydrofuran (10 mL) under nitrogen atmosphere was added lithium aluminum deuteride (8.75 mg, 0.208 mmol, 2.5 equiv). The reaction mixture was allowed to warm to room temperature and the resulting mixture was stirred for additional 2 h. The reaction was adjusted pH to 7 with 1M hydrochloric acid and the resulting mixture was extracted with ethyl acetate (3×30 mL). The combined organic layers were washed with brine (30 mL), dried over anhydrous sodium sulfate, filtered and concentrated under reduced pressure to afford (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-4-(benzyloxy)-3-(formyl-d)benzamide (50 mg, 91%) as a white solid. MS (ESI) calcd. for C₃₉H₃₁DN₈O₃: 661.27 m/z, found 662.30 [M+H]⁺.

### Step 3: (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-3-(formyl-d)-4-hydroxybenzamide (Example 183)

To a solution of (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-4-(benzyloxy)-3-(formyl-d)benzamide (50 mg, 0.076 mmol) in 2,2,2-trifluoroacetic acid (1.5 mL) was added methanesulfonic acid (0.5 mL) and the resulting mixture was stirred for 15 min at room temperature. The mixture was concentrated in vacuo and purified by preparative HPLC on a XBridge Shield RP18 OBD Column using a 25-55% gradient of acetonitrile in water (+10 mmol/L ammonium bicarbonate) to afford (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-3-(formyl-d)-4-hydroxybenzamide (Example 183) (5.6 mg, 12%). MS (ESI) calcd. for C₃₂H₂₅DN₈O₃: 571.20 m/z, found 572.20 [M+H]⁺. ¹H NMR (400 MHz, DMSO-d₆) δ (ppm): 8.32-8.33 (m, 1H), 8.09-8.10 (m, 1H), 7.99-8.00 (m, 1H), 7.78-7.80 (m, 3H), 7.25-7.33 (m, 4H), 6.44-6.53 (m, 3H), 5.58-5.60 (m, 1H), 2.99-3.00 (m, 1H), 2.86-2.88 (m, 1H), 2.67-2.72 (m, 3H), 2.50-2.51 (m, 1H), 2.05-2.10 (m, 1H).

### Intermediate 183-1: 4-(benzyloxy)-3-(methoxy(methyl)carbamoyl)benzoic acid

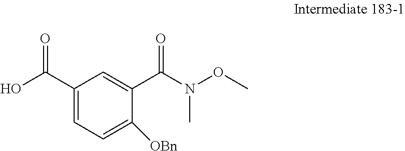 [SMILES: CON(C)C(=O)c1cc(C(=O)O)ccc1OCc1ccccc1]

### Synthetic Route:

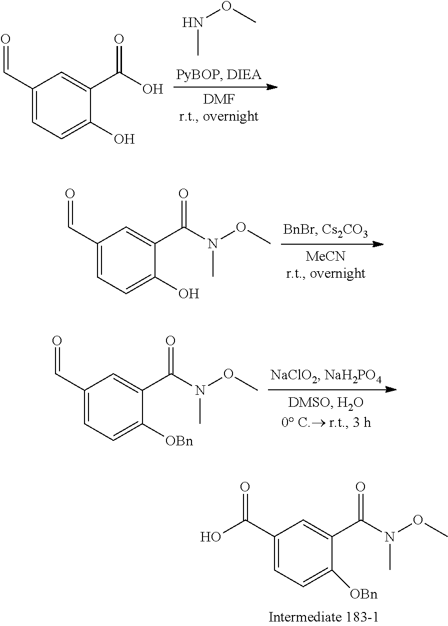

### Step 1: Synthesis of 5-formyl-2-hydroxy-N-methoxy-N-methylbenzamide

To a stirred solution of N,O-dimethylhydroxylamine hydrochloride (132.10 g, 1.354 mol, 5 equiv) in N,N-dimethylformamide (800 mL) was added N,N-diisopropylethylamine (175.04 g, 1.354 mol, 5 equiv) in portions. The resulting mixture was stirred for 30 min at room temperature then 5-formyl-2-hydroxybenzoic acid (45.0 g, 270 mmol, 1 equiv) and PyBOP (140.96 g, 270.87 mmol, 1 equiv) were added and stirring was continued overnight. The reaction was quenched with water (2000 mL). The mixture was acidified to pH 5-6 with 1M hydrochloric acid then extracted with dichloromethane (3×500 mL). The combined organic layers were washed with brine (3×200 mL), dried over anhydrous sodium sulfate, filtered and concentrated under reduced pressure to afford 5-formyl-2-hydroxy-N-methoxy-N-methylbenzamide (35 g, 62%) as yellow oil. MS (ESI) calcd. for C₁₀H₁₁NO₄: 209.20 m/z, found 208.10 [M−H]⁻.

### Step 2: Synthesis of 2-(benzyloxy)-5-formyl-N-methoxy-N-methylbenzamide

To a mixture of 5-formyl-2-hydroxy-N-methoxy-N-methylbenzamide (35 g, 167 mmol) and Cs₂CO₃ (141 g, 432 mmol, 2.5 equiv) in acetonitrile (450 mL) and N,N-dimethylformamide (250 mL) was added benzyl bromide (58.86 g, 344.1 mmol, 2 equiv) and the resulting mixture was stirred overnight at room temperature. The mixture was filtered, rinsing with ethyl acetate (3×500 mL). The filtrate was partially concentrated under reduced pressure and washed with water and brine, dried over anhydrous sodium sulfate, filtered and was concentrated under reduced pressure. The residue was purified by silica gel column chromatography using a 0-20% gradient of ethyl acetate in petroleum ether to afford 2-(benzyloxy)-5-formyl-N-methoxy-N-methylbenzamide (30 g, 58%) as a yellow solid. MS (ESI) calcd. for C₁₇H₁₇NO₄: 299.12 m/z, found 300.15 [M+H]⁺.

### Step 3: 4-(benzyloxy)-3-[methoxy(methyl)carbamoyl]benzoic acid (Intermediate 183-1)

To a cooled (0° C.) solution of 2-(benzyloxy)-5-formyl-N-methoxy-N-methylbenzamide (1.00 g, 3.34 mmol, 1 equiv) in water (10 mL) and DMSO (15 mL) were added monobasic sodium phosphate (1.60 g, 13.3 mmol, 4 equiv) and sodium chlorite (1.21 g, 13.3 mmol, 4 equiv). The resulting mixture was stirred at room temperature for 3 h then quenched with water (50 mL) and extracted with ethyl acetate (100 mL×3). The combined extracts were dried over sodium sulfate, filtered and concentrated under vacuum to give 4-(benzyloxy)-3-[methoxy(methyl)carbamoyl]benzoic acid (Intermediate 183-1) (900 mg, 85%) as a yellow solid. MS (ESI) calcd. for C₁₇H₁₇NO₅: 315.11 m/z, found 316.10 [M+H]⁺.

### Example 184: 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-4-oxoquinazoline-6-carbaldehyde

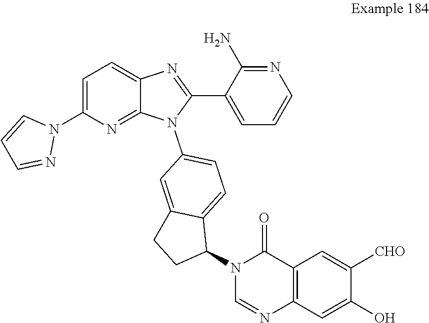 [SMILES: Nc1ncccc1-c1nc2ccc(-n3cccn3)nc2n1-c1ccc2c(c1)CC[C@@H]2n1cnc2cc(O)c(C=O)cc2c1=O]

### Synthetic Route:

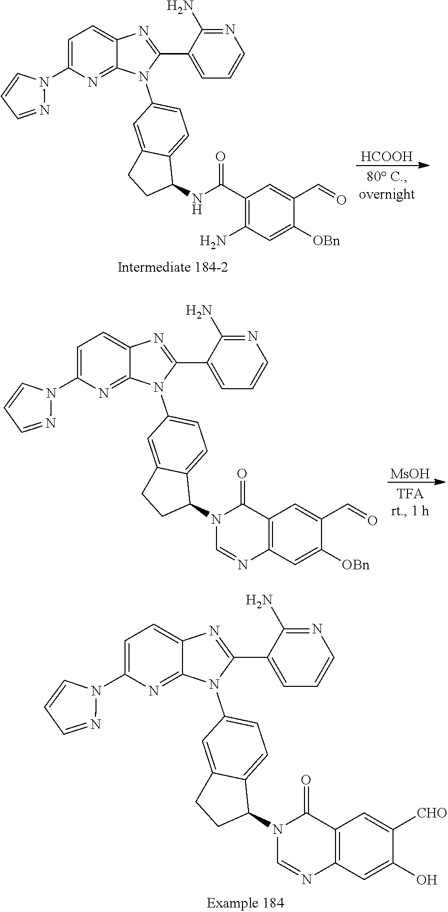

### Step 1: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-4-oxoquinazoline-6-carbaldehyde

A solution of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-formylbenzamide (Intermediate 184-2) (80 mg, 0.12 mmol) in formic acid (6 mL) was stirred at 80° C. overnight. The solvent was removed by distillation under vacuum and the crude residue was purified by reverse-phase flash column chromatography on C18 silica gel using a 20-70% gradient of acetonitrile in water (+0.05% ammonium bicarbonate) to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-4-oxoquinazoline-6-carbaldehyde (70 mg, 86%) as a yellow solid. MS (ESI) calcd. for C₃₉H₂₉N₉O₃: 671.24 m/z, found 672.30 [M+H]⁺.

### Step 2: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-4-oxoquinazoline-6-carbaldehyde (Example 184)

To a solution of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-4-oxoquinazoline-6-carbaldehyde (60 mg, 0.089 mmol) in 2,2,2-trifluoroacetic acid (0.9 mL) was added methanesulfonic acid (0.3 mL) and the resulting mixture was stirred at room temperature for 1 h. The crude product was then purified by preparative HPLC on a XSelect CSH Prep C18 OBD Column using a 17-39% gradient of acetonitrile in water (+0.1% formic acid) to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-4-oxoquinazoline-6-carbaldehyde (Example 184) (11 mg, 22%) as an off-white solid. MS (ESI) calcd. for C₃₂H₂₃N₉O₃: 581.19 m/z, found 582.30 [M+H]⁺. ¹H-NMR (400 MHz, DMSO-d₆) δ (ppm): 10.31 (s, 1H), 8.50 (s, 1H), 8.35-8.39 (m, 2H), 8.11-8.12 (m, 1H), 8.00-8.01 (m, 1H), 7.95-7.97 (m, 1H), 7.81 (s, 1H), 7.51 (s, 1H), 7.32-7.34 (m, 1H), 7.25-7.27 (m, 1H), 7.21-7.24 (m, 1H), 7.11 (s, 1H), 6.55-6.56 (m, 1H), 6.44-6.47 (m, 1H), 6.27-6.30 (m, 1H), 3.19-3.28 (m, 1H), 3.03-3.05 (m, 1H), 2.68-2.73 (m, 1H), 2.34-2.38 (m, 1H).

### Intermediate 184-2: (S)-2-amino-N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-5-formylbenzamide

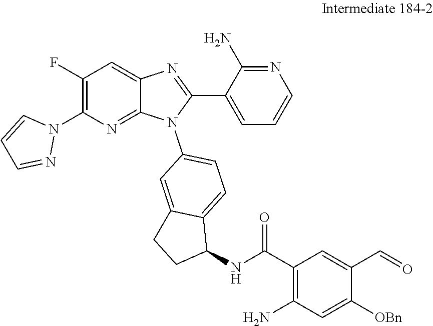 [SMILES: Nc1cc(OCc2ccccc2)c(C=O)cc1C(=O)N[C@H]1CCc2cc(-n3c(-c4cccnc4N)nc4cc(F)c(-n5cccn5)nc43)ccc21]

Intermediate 184-2 was prepared in a manner analogous to Example 12 using Intermediate 33-1 in place of Intermediate 1-3, Intermediate 184-1 in place of Intermediate 12-1 and dichloromethane/2,2,2-trifluoroacetic acid (3:1) in place of 2,2,2-trifluoroacetic acid/methanesulfonic acid. Purification of the crude product was carried out as follows: the solvent was removed by distillation under vacuum and the residue was purified by silica gel column chromatography to afford 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-formylbenzamide (Intermediate 184-1) (240 mg, 65%) as a yellow solid. MS (ESI) calcd. for C₃₈H₃₁N₉O₃: 661.25 m/z, found 662.30 [M+H]⁺.

### Intermediate 184-1: 4-(benzyloxy)-2-((tert-butoxycarbonyl)amino)-5-(1,3-dioxolan-2-yl)benzoic acid

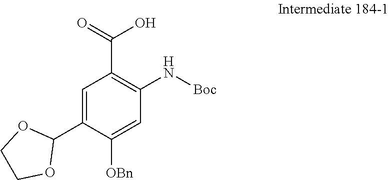 [SMILES: CC(C)(C)OC(=O)Nc1cc(OCc2ccccc2)c(C2OCCO2)cc1C(=O)O]

Intermediate 184-1 was prepared according to the synthetic route outlined below. Steps 1, 2, 3 and 5 were carried out in a manner analogous to Steps 1, 2, 3 and 4, respectively, from the preparation of Intermediate 93-1 using methyl 2-bromo-4-hydroxybenzoate in place of 3-chloro-4-hydroxybenzoic acid. Step 4 is detailed below. MS (ESI) calcd. for C₂₂H₂₅NO₇: 415.16 m/z, found 416.15 [M+H]⁺. It should be noted that the formylation carried out in step 1 produced 2 regioisomers which were separated on silica gel using a 0-5% gradient of ethyl acetate in petroleum ether.

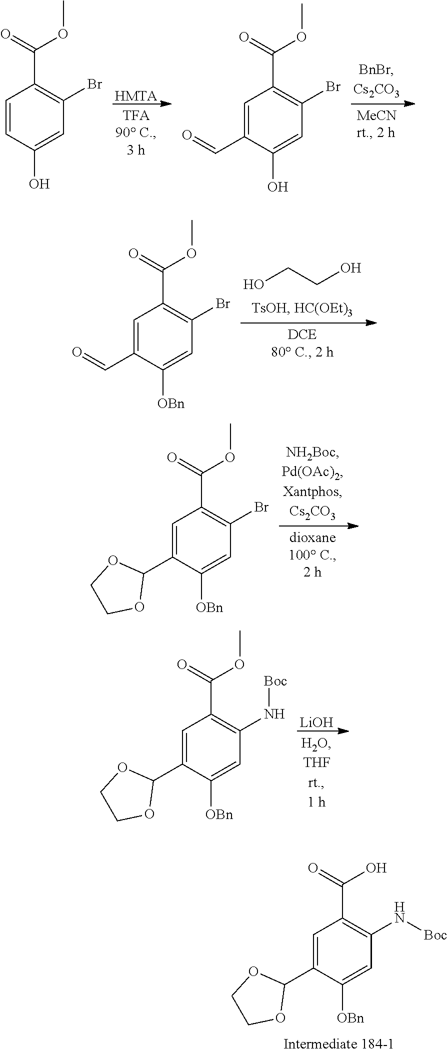

### Step 4: Synthesis of methyl 4-(benzyloxy)-2-[(tert-butoxycarbonyl)amino]-5-(1,3-dioxolan-2-yl)benzoate

To a solution of methyl 4-(benzyloxy)-2-bromo-5-(1,3-dioxolan-2-yl)benzoate (380 mg, 0.966 mmol, 1 equiv) in 1,4-dioxane (12 mL) was added tert-butyl carbamate (170 mg, 1.45 mmol, 1.5 equiv), palladium (II) acetate (22 mg, 0.097 mmol, 0.1 equiv), XantPhos (112 mg, 0.193 mmol, 0.2 equiv) and cesium carbonate (945 mg, 2.90 mmol, 3 equiv). The resulting mixture was stirred at 100° C. for 2 h under nitrogen atmosphere. The mixture was cooled to room temperature and quenched with water (50 mL). The resulting mixture was extracted with ethyl acetate (3×50 mL). The organic layers were combined, dried over sodium sulfate, filtered and concentrated. The residue obtained was purified by silica gel column chromatography using a 0-10% gradient of ethyl acetate in petroleum ether to afford methyl 4-(benzyloxy)-2-[(tert-butoxycarbonyl)amino]-5-(1,3-dioxolan-2-yl)benzoate (320 mg, 77%) as a yellow solid. MS (ESI) calcd. for C₂₃H₂₇NO₇: 429.18 m/z, found 430.20 [M+H]⁺.

### Example 185: N-[(1S)-5-[2-(2-aminopyridin-3-yl)-5-(5-methyl-1,3-thiazol-2-yl)imidazo[4,5-b]pyridin-3-yl]-2,3-dihydro-1H-inden-1-yl]-2-fluoro-5-formyl-4-hydroxybenzamide

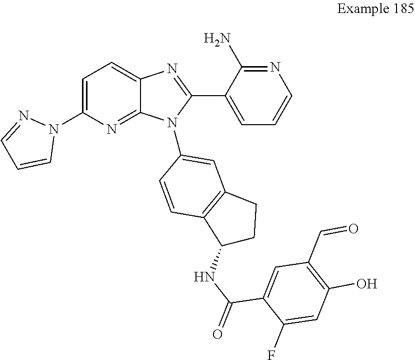 [SMILES: Nc1ncccc1-c1nc2ccc(-n3cccn3)nc2n1-c1ccc2c(c1)CC[C@@H]2NC(=O)c1cc(C=O)c(O)cc1F]

Example 185 was prepared in a manner analogous to Example 7 using Intermediate 185-1 in place of Intermediate 1-2 and Intermediate 16-1 in place of Intermediate 5-1. MS (ESI) calcd. for C₃₂H₂₄FN₇O₃S: 605.65 m/z, found 606.10 [M+H]⁺. ¹H NMR (400 MHz, DMSO-d₆) δ (ppm): 10.21 (s, 1H), 8.25-8.41 (m, 1H), 8.10-8.20 (m, 1H), 7.95-8.10 (m, 2H), 7.55-7.75 (m, 1H), 7.25-7.50 (m, 4H), 6.75-6.95 (m, 1H), 6.35-6.55 (m, 1H), 5.50-5.70 (m, 1H), 2.85-3.05 (m, 2H), 2.56 (s, 3H), 2.55 (m, 1H), 1.95-2.16 (m, 1H). ¹⁹F NMR (376 MHz, DMSO-d₆) δ (ppm): −101.78.

### Intermediate 185-1: (S)-3-(3-(1-amino-2,3-dihydro-1H-inden-5-yl)-5-(5-methylthiazol-2-yl)-3H-imidazo[4,5-b]pyridin-2-yl)pyridin-2-amine

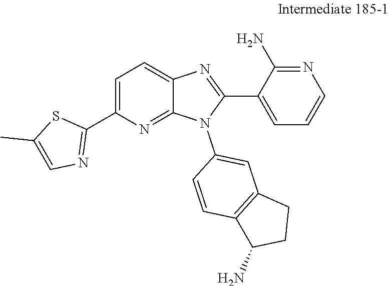 [SMILES: Cc1cnc(-c2ccc3nc(-c4cccnc4N)n(-c4ccc5c(c4)CC[C@@H]5N)c3n2)s1]

Intermediate 185-1 was prepared in a manner analogous to Intermediate 57-1 using 5-methyl-2-(tributylstannyl)-1,3-thiazole in place of 2-(tributylstannyl)-1,3-thiazole. MS (ESI) calcd. for C₂₄H₂₁N₇S: 439.16 m/z, found 440.20 [M+H]⁺.

### Example 186: N-[(1S)-5-[2-(2-aminopyridin-3-yl)-5-(5-methyl-1,3-thiazol-2-yl)imidazo[4,5-b]pyridin-3-yl]-2,3-dihydro-1H-inden-1-yl]-2,3-difluoro-5-formyl-4-hydroxybenzamide

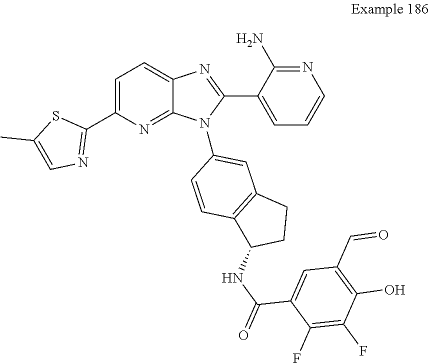 [SMILES: Cc1cnc(-c2ccc3nc(-c4cccnc4N)n(-c4ccc5c(c4)CC[C@@H]5NC(=O)c4cc(C=O)c(O)c(F)c4F)c3n2)s1]

Example 186 was prepared in a manner analogous to Example 7 using Intermediate 185-1 in place of Intermediate 1-2 and Intermediate 118-1 in place of Intermediate 5-1. MS (ESI) calcd. for C₃₂H₂₃F₂N₇O₃S: 623.16 m/z, found 624.20 [M+H]⁺. ¹H NMR (400 MHz, DMSO-d₆) δ (ppm): 10.10 (s, 1H), 8.22-8.24 (m, 1H), 8.06-8.08 (m, 1H), 7.95-7.97 (m, 1H), 7.74-7.76 (m, 1H), 7.55-7.56 (m, 1H), 7.23-7.37 (m, 4H), 6.49-6.52 (m, 1H), 5.48-5.52 (m, 1H), 2.81-3.00 (m, 2H), 2.50-2.51 (m, 1H), 2.34-2.39 (m, 3H), 1.97-2.04 (m, 1H).

### Example 188: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2,4-difluoro-5-formylbenzamide

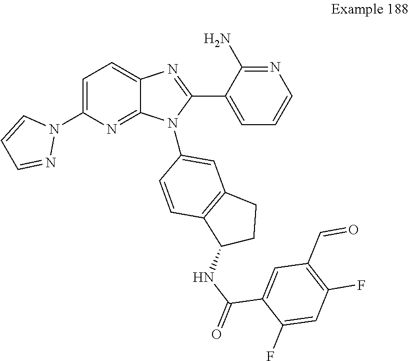 [SMILES: Nc1ncccc1-c1nc2ccc(-n3cccn3)nc2n1-c1ccc2c(c1)CC[C@@H]2NC(=O)c1cc(C=O)c(F)cc1F]

### Synthetic Route:

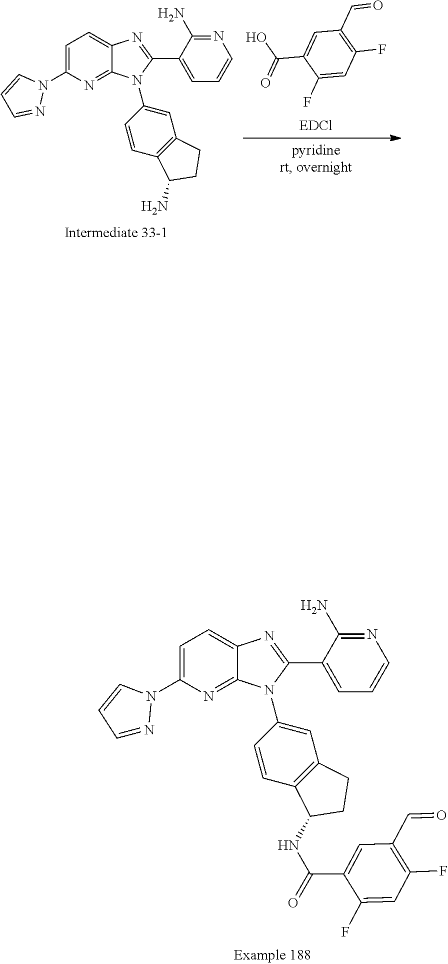

### Step 1: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2,4-difluoro-5-formylbenzamide (Example 188)

To a solution of (S)-3-(3-(1-amino-2,3-dihydro-1H-inden-5-yl)-5-(1H-pyrazol-1-yl)-3H-imidazo[4,5-b]pyridin-2-yl)pyridin-2-amine (Intermediate 33-1) (200 mg, 0.490 mmol, 1 equiv) in pyridine (5 mL) was added 1-ethyl-3-(3-dimethylaminopropyl)carbodiimide (141 mg, 0.735 mmol, 1.5 equiv) and 2,4-difluoro-5-formylbenzoic acid (137 mg, 0.735 mmol, 1.5 equiv). The resulting mixture was stirred overnight at room temperature. The reaction mixture was poured into water (20 mL) and the resulting precipitated solids were collected by filtration. The crude solid was purified by preparative HPLC on a XSelect CSH OBD Column using a 19-43% gradient of acetonitrile in water (+0.1% formic acid) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2,4-difluoro-5-formylbenzamide (Example 188) (12 mg, 4%) as a yellow solid. MS (ESI) calcd. for C₃₁H₂₂F₂N₈O₂: 576.18 m/z, found: 577.10 [M+H]⁺. ¹H NMR (400 MHz, DMSO-d₆) δ (ppm): 10.11 (s, 1H), 9.09-9.11 (m, 1H), 8.30-8.35 (m, 2H), 8.05-8.12 (m, 1H), 7.98-8.00 (m, 1H), 7.91-7.95 (m, 1H), 7.77-7.79 (m, 1H), 7.45-7.51 (m, 1H), 7.35-7.42 (m, 2H), 7.20-7.31 (m, 2H), 6.53 (s, 1H), 6.45-6.51 (m, 1H), 5.51-5.57 (m, 1H), 2.83-3.05 (m, 2H), 2.43-2.47 (m, 1H), 1.91-2.02 (m, 1H). ¹⁹F NMR (376 MHz, DMSO-d₆) δ (ppm): −99.68, −112.12.

### Example 189: N-[(1S)-5-[2-(2-aminopyridin-3-yl)-5-(pyrazol-1-yl)imidazo[4,5-b]pyridin-3-yl]-7-fluoro-2,3-dihydro-1H-inden-1-yl]-3-formyl-4-hydroxybenzamide

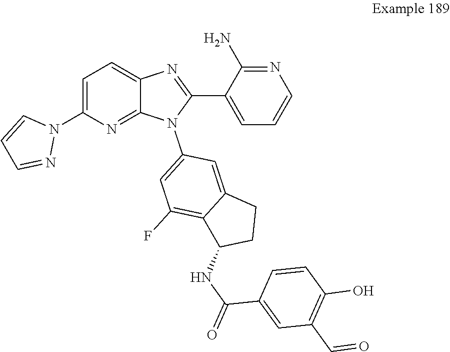 [SMILES: Nc1ncccc1-c1nc2ccc(-n3cccn3)nc2n1-c1cc(F)c2c(c1)CC[C@@H]2NC(=O)c1ccc(O)c(C=O)c1]

Example 189 was prepared in a manner analogous to Example 7 using Intermediate 189-1 in place of Intermediate 1-2. MS (ESI) calcd. for C₃₁H₂₃FN₈O₃: 574.519 m/z, found 575.05 [M+H]⁺. ¹H NMR (400 MHz, DMSO-d₆) δ (ppm): 10.23-10.25 (m, 1H), 8.22-8.49 (m, 2H), 8.15-8.20 (m, 1H), 7.98-8.10 (m, 3H), 7.75-7.95 (m, 1H), 7.50-7.55 (m, 1H), 7.14-7.44 (m, 2H), 6.98-7.13 (m, 1H), 6.50-6.70 (m, 2H), 5.75-5.85 (m, 1H), 3.18-3.24 (m, 1H), 2.81-2.99 (m, 1H), 2.55-2.65 (m, 1H), 1.97-2.15 (m, 1H).

### Intermediate 189-1: (S)-3-(3-(1-amino-7-fluoro-2,3-dihydro-1H-inden-5-yl)-5-(1H-pyrazol-1-yl)-3H-imidazo[4,5-b]pyridin-2-yl)pyridin-2-amine

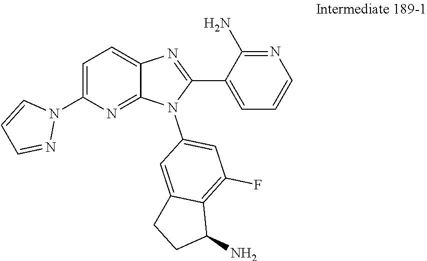 [SMILES: Nc1ncccc1-c1nc2ccc(-n3cccn3)nc2n1-c1cc(F)c2c(c1)CC[C@@H]2N]

### Synthetic Route:

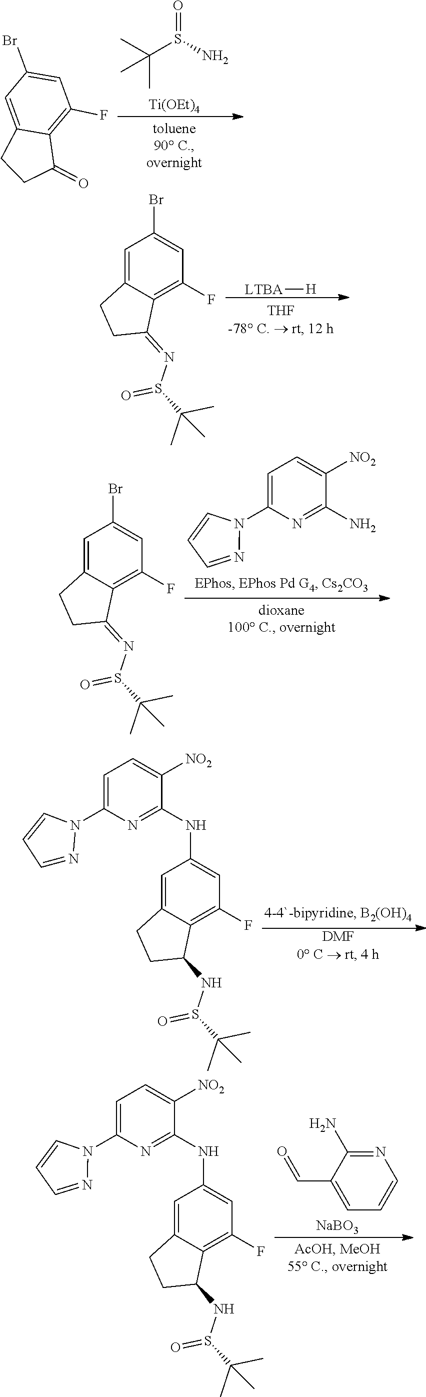

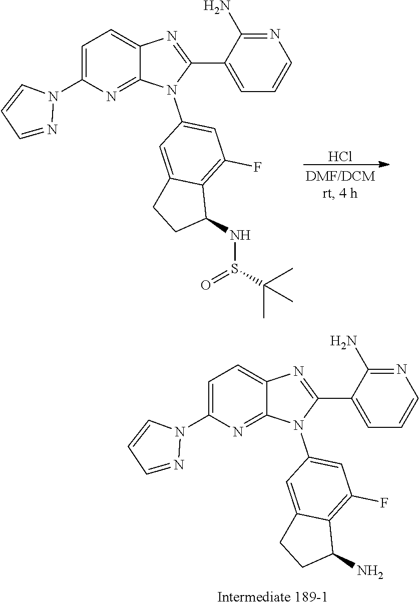 [SMILES: CC(C)(C)[S@](=O)N[C@H]1CCc2cc(-n3c(-c4cccnc4N)nc4ccc(-n5cccn5)nc43)cc(F)c21 ; Cl ; Nc1ncccc1-c1nc2ccc(-n3cccn3)nc2n1-c1cc(F)c2c(c1)CC[C@@H]2N]

### Step 1: (S)—N-(5-bromo-7-fluoro-2,3-dihydroinden-1-ylidene)-2-methylpropane-2-sulfinamide

To a solution of 5-bromo-7-fluoro-2,3-dihydroinden-1-one (5.00 g, 21.8 mmol, 1 equiv) in toluene (150 mL) was added (S)-2-methylpropane-2-sulfinamide (3.17 g, 26.2 mmol, 1.2 equiv) and the resulting mixture was stirred for 30 min at 90° C. under nitrogen atmosphere. The mixture was cooled to room temperature and titanium (IV) isopropoxide (1.6 M, 22 mL, 37 mmol, 1.7 equiv) was added dropwise. The resulting mixture was stirred overnight at 90° C. under nitrogen atmosphere. The mixture cooled to room temperature and brought to pH˜8 with saturated aqueous sodium bicarbonate. The resulting mixture was extracted with ethyl acetate (250×3 mL). The combined organic layers were dried over anhydrous sodium sulfate, filtered and concentrated under reduced pressure to provide (S)—N-(5-bromo-7-fluoro-2,3-dihydroinden-1-ylidene)-2-methylpropane-2-sulfinamide (4.2 g, 58%) as a brown solid, which was used in the next step directly without further purification. MS (ESI) calcd. for C₁₃H₁₅BrFNOS: 331.00 m/z, found 331.90 [M+H]⁺.

### Step 2: (S)—N-(5-bromo-7-fluoro-2,3-dihydro-1H-inden-1-yl)-2-methylpropane-2-sulfinamide

To a cooled (−78° C.) solution of (S)—N-(5-bromo-7-fluoro-2,3-dihydroinden-1-ylidene)-2-methylpropane-2-sulfinamide (4.20 g, 12.6 mmol, 1 equiv) in tetrahydrofuran (300 mL) under nitrogen atmosphere was added lithium tri-tert-butoxyaluminum hydride (4.82 g, 19.0 mmol, 1.5 equiv) in portions. The resulting mixture was allowed to warm to room temperature and was stirred for 12 h. The reaction was quenched by the addition of saturated aqueous ammonium chloride (500 mL) and the resulting mixture was extracted with ethyl acetate (500×3 mL). The combined organic layers were dried over anhydrous sodium sulfate, filtered and concentrated under reduced pressure. The residue was purified by reverse-phase flash column chromatography on C18 silica gel using a 10-50% gradient of acetonitrile in water (+0.1% formic acid) to provide (S)—N-(5-bromo-7-fluoro-2,3-dihydro-1H-inden-1-yl)-2-methylpropane-2-sulfinamide (2.3 g, 45%) as a yellow solid. MS (ESI) calculated for C₁₃H₁₇BrFNOS: 333.02 m/z, found 334.30 [M+H]⁺.

### Step 3: (S)—N-[(1S)-7-fluoro-5-{[3-nitro-6-(pyrazol-1-yl) pyridin-2-yl]amino}-2,3-dihydro-1H-inden-1-yl]-2-methylpropane-2-sulfinamide

A mixture of (S)—N-[(1S)-5-bromo-7-fluoro-2,3-dihydro-1H-inden-1-yl]-2-methylpropane-2-sulfinamide (1.5 g, 4.5 mmol, 1 equiv), 3-nitro-6-(pyrazol-1-yl) pyridin-2-amine (1.10 g, 5.39 mmol, 1.2 equiv), EPhos Pd G4 (0.41 g, 0.45 mmol, 0.1 equiv), EPhos (0.48 g, 0.90 mmol, 0.2 equiv) and cesium carbonate (2.92 g, 8.98 mmol, 2. equiv) in 1,4-dioxane (60 mL) was stirred at 100° C. for 3 h under nitrogen atmosphere. The reaction was cooled to room temperature and quenched by the addition of saturated aqueous ammonium chloride (100 mL). The resulting mixture was extracted with ethyl acetate (200×3 mL). The combined organic layers were dried over anhydrous sodium sulfate, filtered and concentrated under reduced pressure. The residue was purified by reverse-phase flash column chromatography on C18 silica gel using a 10-60% gradient of acetonitrile in water (+0.10% formic acid) to provide (S)—N-[(1S)-7-fluoro-5-{[3-nitro-6-(pyrazol-1-yl) pyridin-2-yl]amino}-2,3-dihydro-1H-inden-1-yl]-2-methylpropane-2-sulfinamide (1.0 g, 47%) as a brown/yellow solid. MS (ESI) calcd. for C₂₁H₂₃FN₆O₃S, 458.15 m/z, found 459.05 [M+H]⁺.

### Step 4: (S)—N-[(1S)-5-{[3-amino-6-(pyrazol-1-yl) pyridin-2-yl]amino}-7-fluoro-2,3-dihydro-1H-inden-1-yl]-2-methylpropane-2-sulfinamide

A cooled (0° C.) solution of (S)—N-[(1S)-7-fluoro-5-{[3-nitro-6-(pyrazol-1-yl) pyridin-2-yl]amino}-2,3-dihydro-1H-inden-1-yl]-2-methylpropane-2-sulfinamide (450 mg, 0.981 mmol, 1 equiv) in N,N-dimethylformamide (10 mL) was treated with tetrahydroxydiboron (264 mg, 2.94 mmol, 3 equiv). The resulting mixture was stirred for 3 min at 0° C. under nitrogen atmosphere followed by addition of 4,4′-dipyridyl (7.7 mg, 0.049 mmol, 0.05 equiv) in portions. The solution was stirred for 4 h at room temperature then quenched by the addition of saturated aqueous ammonium chloride (10 mL). The resulting mixture was extracted with ethyl acetate (20×3 mL). The combined organic layers were dried over anhydrous sodium sulfate, filtered and concentrated under reduced pressure. The residue was purified by reverse-phase flash column chromatography on C18 silica gel using a 10-50% gradient of acetonitrile in water (+0.1% formic acid) to provide (S)—N-[(1S)-5-{[3-amino-6-(pyrazol-1-yl) pyridin-2-yl]amino}-7-fluoro-2,3-dihydro-1H-inden-1-yl]-2-methylpropane-2-sulfinamide (360 mg, 84%) as a yellow solid. MS (ESI) calcd. for C₂₁H₂₅FN₆OS: 428.18 m/z, found 451.15 [M+Na]⁺.

### Step 5: (S)—N-[(1S)-5-[2-(2-aminopyridin-3-yl)-5-(pyrazol-1-yl) imidazo[4,5-b]pyridin-3-yl]-7-fluoro-2,3-dihydro-1H-inden-1-yl]-2-methylpropane-2-sulfinamide

To a solution of (S)—N-[(1S)-5-{[3-amino-6-(pyrazol-1-yl) pyridin-2-yl]amino}-7-fluoro-2,3-dihydro-1H-inden-1-yl]-2-methylpropane-2-sulfinamide (340 mg, 0.793 mmol, 1 equiv) in methanol (3 mL) was added with 2-aminopyridine-3-carbaldehyde (194 mg, 1.59 mmol, 2 equiv) acetic acid (3 mL) and sodium perborate (242 mg, 2.38 mmol, 3 equiv) in portions at room temperature. The resulting mixture was stirred overnight at 55° C. The mixture was concentrated in vacuo and taken up into saturated aqueous sodium bicarbonate. The resulting mixture was extracted with ethyl acetate (20×3 mL). The combined organic layers were dried over anhydrous sodium sulfate, filtered, and concentrated under reduced pressure. The residue was purified by silica gel column chromatography using a 0-35% gradient of ethyl acetate in petroleum ether to afford (S)—N-[(1S)-5-[2-(2-aminopyridin-3-yl)-5-(pyrazol-1-yl) imidazo[4,5-b]pyridin-3-yl]-7-fluoro-2,3-dihydro-1H-inden-1-yl]-2-methylpropane-2-sulfinamide (150 mg, 33%) as a yellow solid. MS (ESI) calcd. for C₂₇H₂₇FN₈OS: 530.63 m/z, found: 531.25 [M+H]⁺.

### Step 6: 3-{3-[(1S)-1-amino-7-fluoro-2,3-dihydro-1H-inden-5-yl]-5-(pyrazol-1-yl) imidazo[4,5-b]pyridin-2-yl}pyridin-2-amine (Intermediate 189-1)

A solution of (S)—N-[(1S)-5-[2-(2-aminopyridin-3-yl)-5-(pyrazol-1-yl) imidazo [4,5-b] pyridin-3-yl]-7-fluoro-2,3-dihydro-1H-inden-1-yl]-2-methylpropane-2-sulfinamide (100 mg, 0.188 mmol, 1 equiv) in dichloromethane (1.2 mL) was treated with N,N-dimethylformamide (0.9 mL) for 3 min at room temperature under nitrogen atmosphere followed by the addition of hydrochloric acid (1.2 mL, concentrated) dropwise. The solution was stirred for 4 h at room temperature. The resulting mixture was concentrated to dryness under reduced pressure. The residue was taken up into saturated aqueous sodium bicarbonate and 3:1 chloroform/isopropanol. The layers were separated and the aqueous layer was extracted twice with 3:1 chloroform/isopropanol. The combined organic layers were dried over anhydrous sodium sulfate, filtered and concentrated under reduced pressure to provide 3-{3-[(1S)-1-amino-7-fluoro-2,3-dihydro-1H-inden-5-yl]-5-(pyrazol-1-yl)imidazo[4,5-b]pyridin-2-yl}pyridin-2-amine (Intermediate 189-1) (120 mg, 93% yield) as a yellow solid, which was used without further purification in subsequent transformations. MS (ESI) calcd. for C₂₃H₁₉FN₈: 426.17 m/z, found 427.20 [M+H]⁺.

### Example 190: (S)—N-(5-(2-(2-aminopyridin-3-yl)-5-(3-fluoro-1H-pyrazol-1-yl)-3H-imidazo[4,5-b]pyridin-3-yl)-2,3-dihydro-1-H-inden-1-yl)-3-(formyl-d)-4-hydroxybenzamide

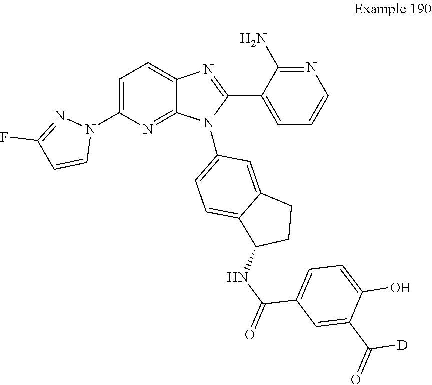 [SMILES: [2H]C(=O)c1cc(C(=O)N[C@H]2CCc3cc(-n4c(-c5cccnc5N)nc5ccc(-n6ccc(F)n6)nc54)ccc32)ccc1O]

Example 190 was prepared in a manner analogous to Example 164 using Example 75 as the starting material instead of Example 33. MS (ESI) calcd. for C₃₁H₂₂DFN₈O₃: 575.19 m/z, found 576.25 [M+H]⁺. ¹H NMR (400 MHz, DMSO-d₆) δ (ppm): 8.90-9.03 (m, 1H), 8.36-8.42 (m, 1H), 8.30-8.36 (m, 2H), 8.08-8.17 (m, 1H), 7.90-8.03 (m, 1H), 7.75-7.82 (m, 1H), 7.32-7.42 (m, 2H), 7.22-7.32 (m, 2H), 7.01-7.11 (m, 1H), 6.40-6.53 (m, 1H), 6.35-6.40 (m, 1H), 5.58-5.68 (m, 1H), 2.98-3.11 (m, 1H), 2.85-2.99 (m, 1H), 2.35-2.37 (m, 1H), 2.05-2.15 (m, 1H).

### Example 191: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-fluoro-3-formylbenzamide

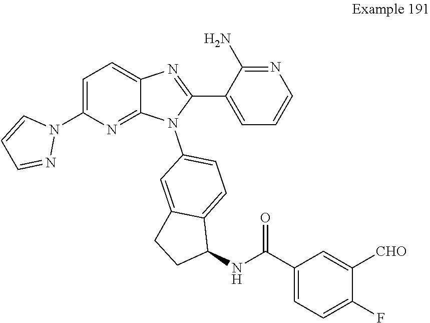 [SMILES: Nc1ncccc1-c1nc2ccc(-n3cccn3)nc2n1-c1ccc2c(c1)CC[C@@H]2NC(=O)c1ccc(F)c(C=O)c1]

### Synthetic Route:

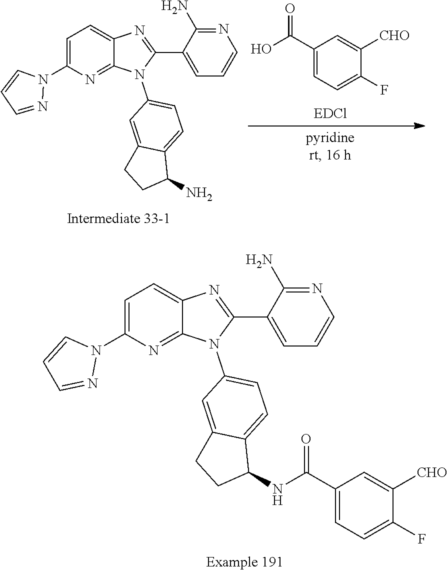

### Step 1: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-fluoro-3-formylbenzamide (Example 191)

To a solution of 4-fluoro-3-formylbenzoic acid (103 mg, 0.612 mmol, 1 equiv) in pyridine (3 mL) was added (S)-3-(3-(1-amino-2,3-dihydro-1H-inden-5-yl)-5-(1H-pyrazol-1-yl)-3H-imidazo[4,5-b]pyridin-2-yl)pyridin-2-amine (Intermediate 33-1) (250 mg, 0.612 mmol, 1 equiv) and 1-ethyl-3-(3-dimethylaminopropyl)carbodiimide (176 mg, 0.918 mmol, 1.5 equiv). The resulting mixture was stirred for 16 h at room temperature then purified by preparative HPLC on a XSelect CSH Prep C18 OBD Column using a 18-40% gradient of acetonitrile in water (+0.1% formic acid) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-fluoro-3-formylbenzamide (Example 191) (30 mg, 8.6%) as a yellow solid. MS (ESI) calcd. for C₃₁H₂₃FN₈O₂: 558.19 m/z, found 559.20 [M+H]⁺. ¹H NMR (400 MHz, DMSO-d₆) δ (ppm): 10.26 (s, 1H), 9.09-9.32 (m, 1H), 8.15-8.57 (m, 4H), 7.88-8.12 (m, 2H), 7.82 (s, 1H), 7.13-7.66 (m, 5H), 6.30-6.72 (m, 2H), 5.49-5.78 (m, 1H), 2.81-3.16 (m, 2H), 2.38-2.46 (m, 1H), 1.98-2.21 (m, 1H). ¹⁹F NMR (376 MHz, DMSO-d₆) δ (ppm): −116.75.

### Example 192: N-[(1S)-5-[2-(2-aminopyridin-3-yl)-5-(pyrazol-1-yl)imidazo[4,5-b]pyridin-3-yl]-2,3-dihydro-1H-inden-1-yl]-3-[(dimethylamino)methyl]-5-formyl-4-hydroxybenzamide

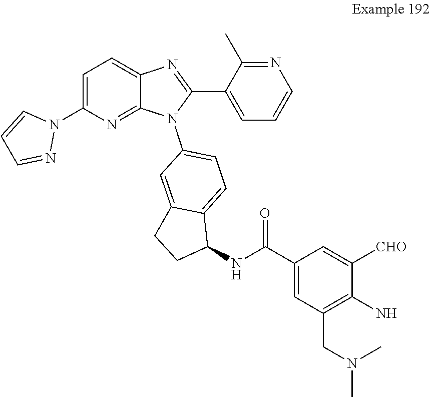 [SMILES: Cc1ncccc1-c1nc2ccc(-n3cccn3)nc2n1-c1ccc2c(c1)CC[C@@H]2NC(=O)c1cc(C=O)c(N)c(CN(C)C)c1]

Example 192 was prepared in a manner analogous to Example 7 using Intermediate 33-1 in place of Intermediate 1-2 and Intermediate 192-1 in place of Intermediate 5-1. MS (ESI) calcd. for C₃₄H₃₁N₉O₃: 613.25 m/z, found 614.35 [M+H]⁺. ¹H NMR (400 MHz, DMSO-d₆) δ (ppm): 10.26 (s, 1H), 8.35-8.37 (m, 2H), 8.13-8.18 (m, 1H), 8.01-8.03 (m, 1H), 7.95-7.97 (m, 1H), 7.81-7.84 (m, 2H), 7.34-7.37 (m, 1H), 7.24-7.32 (m, 3H), 6.57-6.58 (m, 1H), 6.48-6.56 (m, 1H), 5.58-5.61 (m, 1H), 4.06-4.25 (m, 2H), 3.00-3.02 (m, 1H), 2.88-2.91 (m, 1H), 2.85-2.86 (m, 6H), 2.48-2.49 (m, 1H), 2.06-2.11 (m, 1H).

### Intermediate 192-1: 4-(benzyloxy)-3-((dimethylamino)methyl)-5-(1,3-dioxolan-2-yl)benzoic acid

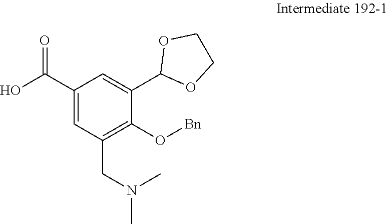 [SMILES: CN(C)Cc1cc(C(=O)O)cc(C2OCCO2)c1OCc1ccccc1]

### Synthetic Route:

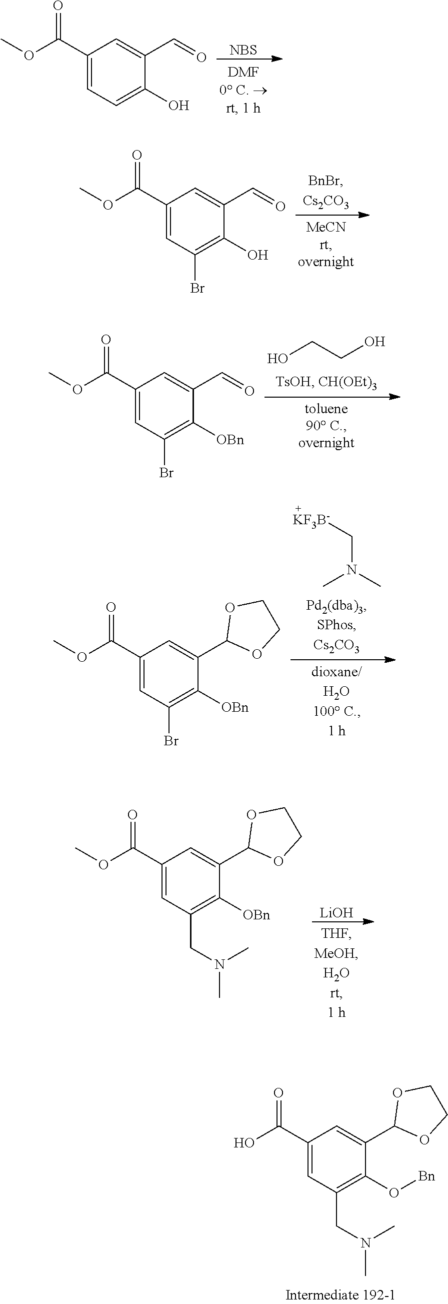

### Step 1: Synthesis of methyl 3-bromo-5-formyl-4-hydroxybenzoate

To a cooled (0° C.) solution of methyl 3-formyl-4-hydroxybenzoate (5.00 g, 27.8 mmol, 1 equiv) in N,N-dimethylformamide (45 mL) was added a solution of N-bromosuccinimide (5.43 g, 30.5 mmol, 1.1 equiv) in N,N-dimethylformamide (15 mL) dropwise over 40 minutes. Stirring was continued for 10 minutes at 0° C. then the mixture was allowed to warm to room temperature and stirred for an additional 1 h. Water (500 mL) was added and the precipitated solids were collected by filtration then dissolved in ethyl acetate, dried over anhydrous sodium sulfate, filtered and concentrated under reduced pressure. The crude product was purified by silica gel column chromatography using a 0-30% gradient of ethyl acetate in petroleum ether to afford methyl 3-bromo-5-formyl-4-hydroxybenzoate (5 g, 69%) as a light-yellow solid. MS (ESI) calcd. for C₉H₇BrO₄: 257.95 m/z, found 256.75 [M−H]⁻.

### Step 2: Synthesis of methyl 4-(benzyloxy)-3-bromo-5-formylbenzoate

To a mixture of methyl 3-bromo-5-formyl-4-hydroxybenzoate (2.5 g, 9.7 mmol, 1 equiv) and cesium carbonate (6.29 g, 19.3 mmol, 2 equiv) in acetonitrile (40 mL) was added benzyl bromide (4.95 g, 29.0 mmol, 3 equiv) and the resulting mixture was stirred at 25° C. overnight. The reaction mixture was quenched with water (40 mL) and extracted with ethyl acetate (100 mL×2). The combined organic layers were washed with water, dried over anhydrous sodium sulfate, filtered, and concentrated under reduced pressure. The crude material was purified by silica gel column chromatography using a 0-10% gradient of ethyl acetate in petroleum ether to afford methyl 4-(benzyloxy)-3-bromo-5-formylbenzoate (2.8 g, 81%) as a white solid. MS (ESI) calcd. for C₁₆H₁₃BrO₄: 348.00 m/z, found 349.30 [M+H]⁺.

### Step 3: Synthesis of methyl 4-(benzyloxy)-3-bromo-5-(1,3-dioxolan-2-yl)benzoate

A solution of methyl 4-(benzyloxy)-3-bromo-5-formylbenzoate (2.0 g, 5.7 mmol, 1 equiv), ethylene glycol (1.78 g, 28.6 mmol, 5 equiv), triethyl orthoformate (1.74 g, 17.2 mmol, 3 equiv) and p-toluenesulfonic acid (0.20 g, 1.1 mmol, 0.2 equiv) in toluene (50 mL) was stirred at 90° C. for 3 h. The reaction mixture was quenched with water (50 mL) and extracted with ethyl acetate (50 mL×3). The combined organic layers were washed with brine, dried over anhydrous sodium sulfate, filtered and concentrated under reduced pressure. The crude product was purified by silica gel column chromatography using a 0-20% gradient of ethyl acetate in petroleum ether to afford methyl 4-(benzyloxy)-3-bromo-5-(1,3-dioxolan-2-yl)benzoate (1.8 g, 79%) as a white solid. MS (ESI) calcd. for C₁₈H₁₇BrO₅: 392.03 m/z, found 393.10 [M+H]⁺.

### Step 4: Synthesis of methyl 4-(benzyloxy)-3-[(dimethylamino)methyl]-5-(1,3-dioxolan-2-yl)benzoate

To a mixture of methyl 4-(benzyloxy)-3-bromo-5-(1,3-dioxolan-2-yl)benzoate (500 mg, 1.27 mmol, 1 equiv), tris(dibenzylideneacetone)dipalladium(0) (116 mg, 0.127 mmol, 0.1 equiv), SPhos (104 mg, 0.254 mmol, 0.2 equiv) and cesium carbonate (829 mg, 2.54 mmol, 2 equiv) in 1,4-dioxane (8 mL) was added a solution of potassium ((dimethylamino)methyl)trifluoroborate (250 mg, 1.52 mmol, 1.2 equiv) in water (2 mL) and the resulting mixture was stirred at 100° C. for 1 h under nitrogen atmosphere. The reaction mixture was quenched with water (50 mL) and extracted with ethyl acetate (10 mL×2). The combined organic layers were washed with brine, dried over anhydrous sodium sulfate, filtered and concentrated under reduced pressure. The residue was purified by silica gel column chromatography using a 0-30% gradient of ethyl acetate in petroleum ether to afford methyl 4-(benzyloxy)-3-[(dimethylamino)methyl]-5-(1,3-dioxolan-2-yl)benzoate (180 mg, 37%) as a colorless oil. MS (ESI) calcd. for C₂₁H₂₅NO₅: 371.17 m/z, found 372.20 [M+H]⁺.

### Step 5: Synthesis of 4-(benzyloxy)-3-[(dimethylamino)methyl]-5-(1,3-dioxolan-2-yl)benzoic acid (Intermediate 192-1)

To a solution of methyl 4-(benzyloxy)-3-[(dimethylamino)methyl]-5-(1,3-dioxolan-2-yl)benzoate (150 mg, 0.404 mmol, 1 equiv) in tetrahydrofuran (3.2 mL) and methanol (0.8 mL) was added a solution of lithium hydroxide (39 mg, 1.6 mmol, 4 equiv) in water (0.8 mL) and the resulting mixture was stirred at 25° C. for 1 h. The reaction was quenched by the addition of water (1 mL) and the resulting mixture was concentrated under reduced pressure. The crude residue was purified by reverse-phase flash column chromatography on C18 silica gel using a 20-80% gradient of acetonitrile in water (+0.1% formic acid) to afford 4-(benzyloxy)-3-[(dimethylamino)methyl]-5-(1,3-dioxolan-2-yl)benzoic acid (Intermediate 192-1) (130 mg, 87%) as a white solid. MS (ESI) calcd. for C₂₀H₂₃NO₅: 357.16 m/z, found 358.10 [M+H]⁺.

### Example 193: N-[(1S)-5-[2-(2-aminopyridin-3-yl)-5-(5-fluoro-1,3-thiazol-2-yl)imidazo[4,5-b]pyridin-3-yl]-2,3-dihydro-1H-inden-1-yl]-2,3-difluoro-5-formyl-4-hydroxybenzamide

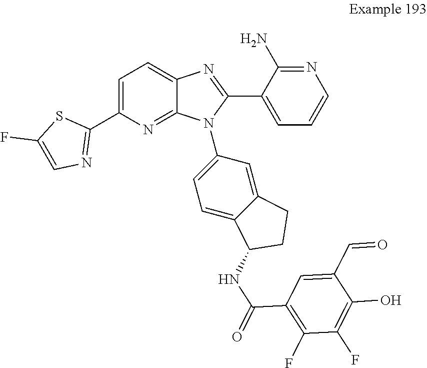 [SMILES: Nc1ncccc1-c1nc2ccc(-c3ncc(F)s3)nc2n1-c1ccc2c(c1)CC[C@@H]2NC(=O)c1cc(C=O)c(O)c(F)c1F]

### Synthetic Route:

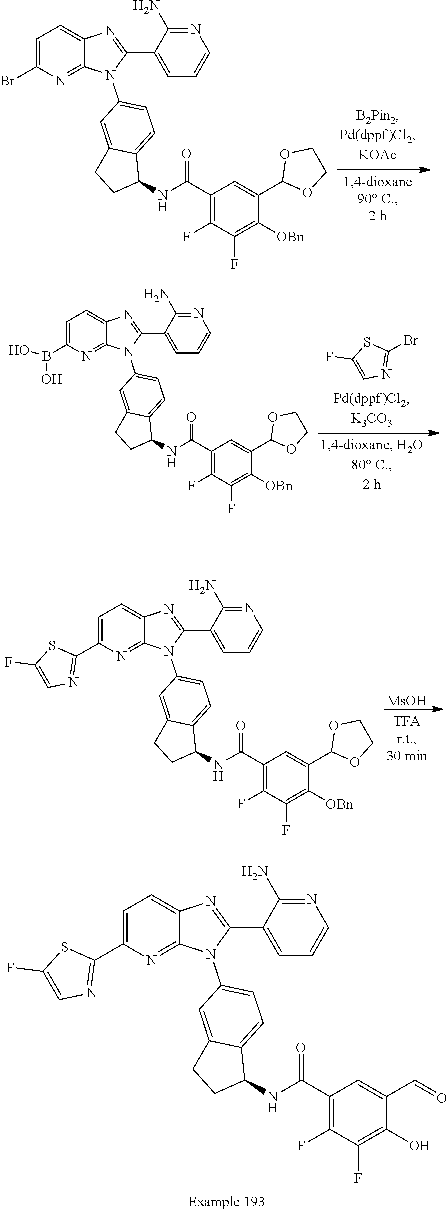

### Step 1: Synthesis of 2-(2-aminopyridin-3-yl)-3-[(1S)-1-[4-(benzyloxy)-5-(1,3-dioxolan-2-yl)-2,3-difluorobenzamido]-2,3-dihydro-1H-inden-5-yl]imidazo[4,5-b]pyridin-5-ylboronic acid

To a solution of N-[(1S)-5-[2-(2-aminopyridin-3-yl)-5-bromoimidazo[4,5-b]pyridin-3-yl]-2,3-dihydro-1H-inden-1-yl]-4-(benzyloxy)-5-(1,3-dioxolan-2-yl)-2,3-difluorobenzamide (Intermediate 193-1) (1.00 g, 1.35 mmol, 1 equiv) in 1,4-dioxane (20 mL) were added bis(pinacolato) diboron (1.03 g, 4.06 mmol, 3 equiv), [1,1′-bis(diphenylphosphino)ferrocene]dichloropalladium(II) (220 mg, 0.270 mmol, 0.2 equiv) and potassium acetate (531 mg, 5.41 mmol, 4 equiv) and the resulting mixture was stirred under nitrogen atmosphere for 2 h at 90° C. The mixture was allowed to cool to room temperature and the reaction was quenched with water (40 mL). The resulting mixture was extracted with ethyl acetate (3×40 mL). The organic layers were combined, dried over anhydrous sodium sulfate, filtered and concentrated. The residue was purified by reverse-phase flash column chromatography on C18 silica gel using a 10-50% gradient of acetonitrile in water (+10 mmol/L ammonium bicarbonate) to afford 2-(2-aminopyridin-3-yl)-3-[(1S)-1-[4-(benzyloxy)-5-(1,3-dioxolan-2-yl)-2,3-difluorobenzamido]-2,3-dihydro-1H-inden-5-yl]imidazo[4,5-b]pyridin-5-ylboronic acid (400 mg, 34%) as a yellow solid. MS (ESI) calcd. for C₃₇H₃₁BF₂N₆O₆: 704.24 m/z, found 705.20 [M+H]⁺.

### Step 2: Synthesis of N-[(1S)-5-[2-(2-aminopyridin-3-yl)-5-(5-fluoro-1,3-thiazol-2-yl)imidazo[4,5-b]pyridin-3-yl]-2,3-dihydro-1H-inden-1-yl]-4-(benzyloxy)-5-(1,3-dioxolan-2-yl)-2,3-difluorobenzamide

To a solution of 2-(2-aminopyridin-3-yl)-3-[(1S)-1-[4-(benzyloxy)-5-(1,3-dioxolan-2-yl)-2,3-difluorobenzamido]-2,3-dihydro-1H-inden-5-yl]imidazo[4,5-b]pyridin-5-ylboronic acid (150 mg, 0.213 mmol, 1 equiv) in 1,4-dioxane (6 mL) and water (1.5 mL) were added 2-bromo-5-fluoro-1,3-thiazole (116 mg, 0.639 mmol, 3 equiv), [1,1′-bis(diphenylphosphino)ferrocene]dichloropalladium(II) (17 mg, 0.021 mmol, 0.1 equiv) and potassium carbonate (88 mg, 0.64 mmol, 3 equiv) and the resulting mixture was stirred for 2 h at 80° C. under nitrogen atmosphere. The mixture was concentrated under reduced pressure and the resulting residue was purified by reverse-phase flash column chromatography on C18 silica gel using a 10-50% gradient of acetonitrile in water (+10 mmol/L ammonium bicarbonate) to afford N-[(1S)-5-[2-(2-aminopyridin-3-yl)-5-(5-fluoro-1,3-thiazol-2-yl)imidazo[4,5-b]pyridin-3-yl]-2,3-dihydro-1H-inden-1-yl]-4-(benzyloxy)-5-(1,3-dioxolan-2-yl)-2,3-difluorobenzamide (50 mg, 26%) as a light-yellow solid. MS (ESI) calcd. for C₄₀H₃₀F₃N₇O₄S: 761.20 m/z, found 762.15 [M+H]⁺.

### Step 3: Synthesis of N-[(1S)-5-[2-(2-aminopyridin-3-yl)-5-(5-fluoro-1,3-thiazol-2-yl)imidazo[4,5-b]pyridin-3-yl]-2,3-dihydro-1H-inden-1-yl]-2,3-difluoro-5-formyl-4-hydroxybenzamide (Example 193)

To a solution of N-[(1S)-5-[2-(2-aminopyridin-3-yl)-5-(5-fluoro-1,3-thiazol-2-yl)imidazo[4,5-b]pyridin-3-yl]-2,3-dihydro-1H-inden-1-yl]-4-(benzyloxy)-5-(1,3-dioxolan-2-yl)-2,3-difluorobenzamide (50 mg, 0.064 mmol) in 2,2,2-trifluoroacetic acid (1.5 mL) was added methanesulfonic acid (0.5 mL) and the resulting mixture was stirred at room temperature for 1 h. The resulting mixture were concentrated under reduced pressure and the crude product was purified by preparative HPLC on a YMC Triart C18 ExRs column using a 23-45% gradient of acetonitrile in water (+10 mmol/L ammonium bicarbonate) to afford N-[(1S)-5-[2-(2-aminopyridin-3-yl)-5-(5-fluoro-1,3-thiazol-2-yl)imidazo[4,5-b]pyridin-3-yl]-2,3-dihydro-1H-inden-1-yl]-2,3-difluoro-5-formyl-4-hydroxybenzamide (Example 193) (6.7 mg, 17%) as a yellow solid. MS (ESI) calcd. for C₃₁H₂₀F₃N₇O₃S: 627.13 m/z, found 628.25 [M+H]⁺. ¹H NMR (400 MHz, DMSO-d₆) δ (ppm): 10.11 (s, 1H), 8.49-8.51 (m, 1H), 8.08-8.09 (m, 1H), 8.01-8.02 (m, 1H), 7.69-7.73 (m, 2H), 7.38-7.35 (m, 2H), 7.27-7.29 (m, 2H), 6.45-6.48 (m, 1H), 5.53-5.54 (m, 1H), 2.97-2.98 (m, 1H), 2.87-2.90 (m, 1H), 2.50-2.51 (m, 1H), 2.02-2.07 (m, 1H). ¹⁹F NMR (376 MHz, DMSO-d₆) δ (ppm): −132.81, −147.94, −161.76.

### Intermediate 193-1: (S)—N-(5-(2-(2-aminopyridin-3-yl)-5-bromo-3H-imidazo[4,5-b]pyridin-3-yl)-2,3-dihydro-1H-inden-1-yl)-4-(benzyloxy)-5-(1,3-dioxolan-2-yl)-2,3-difluorobenzamide

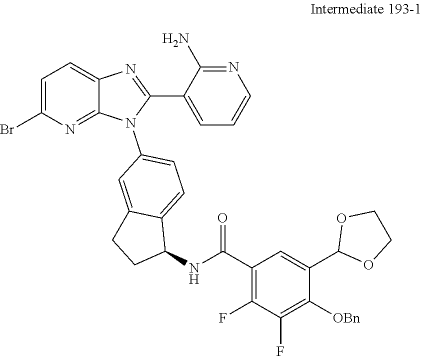 [SMILES: Nc1ncccc1-c1nc2ccc(Br)nc2n1-c1ccc2c(c1)CC[C@@H]2NC(=O)c1cc(C2OCCO2)c(OCc2ccccc2)c(F)c1F]

### Synthetic Route:

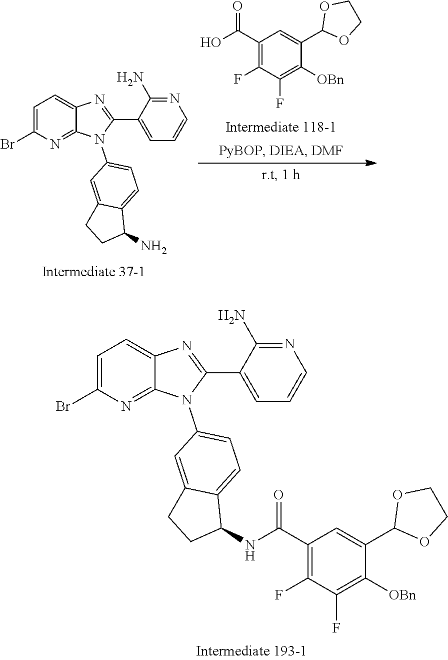 [SMILES: Nc1ncccc1-c1nc2ccc(Br)nc2n1-c1ccc2c(c1)CC[C@@H]2N ; O=C(O)c1cc(C2OCCO2)c(OCc2ccccc2)c(F)c1F ; Nc1ncccc1-c1nc2ccc(Br)nc2n1-c1ccc2c(c1)CC[C@@H]2NC(=O)c1cc(C2OCCO2)c(OCc2ccccc2)c(F)c1F]

### Step 1: Synthesis of N-[(1S)-5-[2-(2-aminopyridin-3-yl)-5-bromoimidazo[4,5-b]pyridin-3-yl]-2,3-dihydro-1H-inden-1-yl]-4-(benzyloxy)-5-(1,3-dioxolan-2-yl)-2,3-difluorobenzamide (Intermediate 193-1)

To a solution of 3-{3-[(1S)-1-amino-2,3-dihydro-1H-inden-5-yl]-5-bromoimidazo[4,5-b]pyridin-2-yl}pyridin-2-amine (Intermediate 37-1) (1.3 g, 3.1 mmol, 1 equiv) in N,N-dimethylformamide (5 mL) was added N,N-diisopropylethylamine (3.99 g, 30.9 mmol, 10 equiv), PyBOP (1.93 g, 3.70 mmol, 1.2 equiv) and 4-(benzyloxy)-5-(1,3-dioxolan-2-yl)-2,3-difluorobenzoic acid (Intermediate 118-1) (1.25 g, 3.70 mmol, 1.2 equiv). The resulting mixture was stirred at room temperature for 1 h. The mixture was purified by reverse-phase flash column chromatography on C18 silica gel using a 20-70% gradient of acetonitrile in water (+0.05% ammonium bicarbonate) to afford N-[(1S)-5-[2-(2-aminopyridin-3-yl)-5-bromoimidazo[4,5-b]pyridin-3-yl]-2,3-dihydro-1H-inden-1-yl]-4-(benzyloxy)-5-(1,3-dioxolan-2-yl)-2,3-difluorobenzamide (Intermediate 193-1) (1.1 g, 47%) as a yellow solid. MS (ESI) calcd. for C₃₇H₂₉BrF₂N₆O₄: 738.14 m/z, found 739.15, 741.10 [M+H, M+H+2]⁺.

### Example 194: N-[(1S)-5-[2-(2-aminopyridin-3-yl)-5-(1H-imidazol-2-yl)imidazo[4,5-b]pyridin-3-yl]-2,3-dihydro-1H-inden-1-yl]-3-formyl-4-hydroxybenzamide

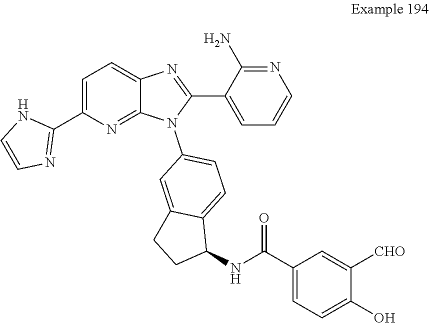 [SMILES: Nc1ncccc1-c1nc2ccc(-c3ncc[nH]3)nc2n1-c1ccc2c(c1)CC[C@@H]2NC(=O)c1ccc(O)c(C=O)c1]

Example 194 was prepared in a manner analogous to Example 193 using Intermediate 165-1 in place of Intermediate 193-1, 2-bromo-1-{[2-(trimethylsilyl)ethoxy]methyl}imidazole in place of 2-bromo-5-fluoro-1,3-thiazole and excluding methanesulfonic acid from the final step. MS (ESI) calcd. for C₃₁H₂₄N₈O₃: 556.20 m/z, found 557.25 [M+H]⁺. ¹H-NMR (400 MHz, DMSO-d₆) δ (ppm): 10.25 (s, 1H), 8.51-8.53 (m, 1H), 8.25-8.26 (m, 1H), 8.16-8.18 (m, 1H), 8.04-8.06 (m, 2H), 7.89-7.91 (m, 1H), 7.70-7.71 (m, 2H), 7.35-7.38 (m, 1H), 7.29-7.33 (m, 2H), 7.20-7.27 (m, 1H), 7.16-7.19 (m, 1H), 6.88-6.90 (m, 1H), 5.54-5.56 (m, 1H), 3.03-3.04 (m, 1H), 2.91-2.93 (m, 1H), 2.50-2.51 (m, 1H), 1.87-2.07 (m, 1H).

### Example 195: (R)—N-(5-(2-(2-aminopyridin-3-yl)-5-(1H-pyrazol-1-yl)-3H-imidazo[4,5-b]pyridin-3-yl)-2,2-difluoro-2,3-dihydro-1H-inden-1-yl)-3-formyl-4-hydroxybenzamide

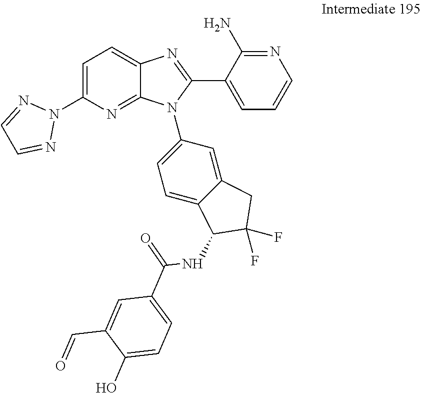 [SMILES: Nc1ncccc1-c1nc2ccc(-n3nccn3)nc2n1-c1ccc2c(c1)CC(F)(F)[C@@H]2NC(=O)c1ccc(O)c(C=O)c1]

Example 195 was prepared in a manner analogous to Example 12 using Intermediate 195-2 in place of Intermediate 1-3, Intermediate 57-2 in place of Intermediate 12-1 and dichloromethane/TFA (5:1) in place of TFA/MsOH. MS (ESI) calcd. for C₃₁H₂₂F₂N₈O₃, 592.18 m/z, found: 593.20[M+H]⁺. ¹H-NMR (400 MHz, DMSO-d6) δ (ppm): 10.32 (s, 1H), 8.36-8.46 (m, 3H), 8.11-8.16 (m, 2H), 8.00-8.09 (m, 1H), 7.74-7.77 (m, 1H), 7.74-7.76 (m, 1H), 7.50-7.57 (m, 1H), 7.45-7.47 (m, 1H), 7.09-7.14 (m, 2H), 6.77-6.82 (m, 1H), 6.59-6.60 (m, 1H), 5.94-6.01 (m, 1H), 3.54-3.68 (m, 3H). (TFA salt).

### Intermediate 195-2: (R)-3-(3-(1-amino-2,2-difluoro-2,3-dihydro-1H-inden-5-yl)-5-(1H-pyrazol-1-yl)-3H-imidazo[4,5-b]pyridin-2-yl)pyridin-2-amine

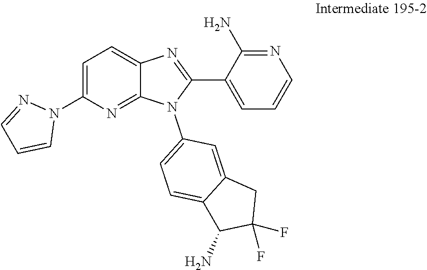 [SMILES: Nc1ncccc1-c1nc2ccc(-n3cccn3)nc2n1-c1ccc2c(c1)CC(F)(F)[C@@H]2N]

Intermediate 195-2 was prepared in a manner analogous to Intermediate 256-1 (starting from Step 2) using Intermediate 195-1 in place of 5-bromo-2-fluoro-2,3-dihydroinden-1-one and omitting the chiral separation by SFC. MS (ESI) calcd. for C₂₃H₁₈F₂N₈, 444.16 m/z, found: 445.15 [M+H]⁺.

### Intermediate 195-1: 5-bromo-2,2-difluoro-2,3-dihydro-1H-inden-1-one

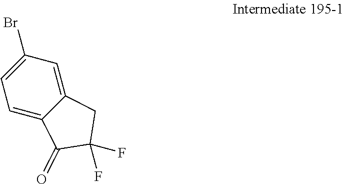 [SMILES: O=C1c2ccc(Br)cc2CC1(F)F]

### Synthetic Route:

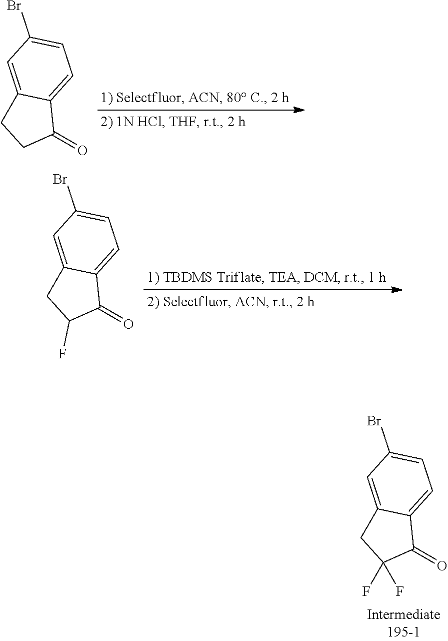

### Step 1: Synthesis of 5-bromo-2-fluoro-2,3-dihydro-1H-inden-1-one

To a solution of 5-bromo-1-indanone (5.00 g, 23.8 mmol) in methanol (50 mL) was added SelectFluor (10.0 g, 28.2 mmol) and the resulting mixture was stirred under reflux for 2 hours. The reaction mixture was filtered through Celite, and the filtrate was concentrated under reduced pressure. The resulting residue was dissolved in tetrahydrofuran (50 mL), and 1 N hydrochloric acid (50 mL) was added followed by stirring at room temperature for 3 hours. To the reaction mixture, a 2 N aqueous sodium hydroxide solution (50 mL) was added, and the mixture was diluted with a saturated aqueous sodium bicarbonate solution. The mixture was extracted with ethyl acetate. The organic layer was washed with brine, dried over anhydrous magnesium sulfate, and then concentrated under reduced pressure to afford 5-bromo-2-fluoro-2,3-dihydroinden-1-one (4.0 g, 74% yield) as a white solid. MS (ESI) calcd. for C₉H₆BrFO, 227.96 m/z, found: 229.00 [M+H]⁺.

### Step 2: 5-bromo-2,2-difluoro-2,3-dihydro-1H-inden-1-one (Intermediate 195-1)

To a solution of 5-bromo-2-fluoro-2,3-dihydro-1H-inden-1-one (3.0 g, 13 mmol, 1 equiv) in DCM (30 mL) was added tert-butyldimethylsilyl trifluoromethanesulfonate (5.16 mL, 988 mmol, 70 equiv) and triethylamine (9.1 mL, 65.5 mmol, 5.0 equiv) and the resulting mixture was stirred at room temperature for 1 hour. The reaction mixture was diluted with saturated aqueous sodium bicarbonate and extracted with ethyl acetate. The organic layer was dried over anhydrous sodium sulfate and concentrated under reduced pressure. The resulting residue was dissolved in acetonitrile (30 mL) and Selectfluor (5.57 g, 15.7 mmol, 1.2 equiv) was added. The resulting mixture was stirred at room temperature for 2 hours. The reaction mixture was diluted with saturated aqueous sodium bicarbonate and extracted with ethyl acetate. The organic layer was washed with brine, dried over anhydrous magnesium sulfate, and concentrated under reduced pressure. To the resulting residue, hexane was added, and the precipitate was collected by filtration to afford 5-bromo-2,2-difluoro-2,3-dihydro-1H-inden-1-one (Intermediate 195-1) (2.3 g, 71%). MS (ESI) calcd. for C₉H₅BrF₂O, 245.95 m/z, found: 247.00 [M+H]⁺.

### Example 196: (S)—N-(5-(2-(2-aminopyridin-3-yl)-7-chloro-5-(1H-pyrazol-1-yl)-3H-imidazo[4,5-b]pyridin-3-yl)-2,3-dihydro-1H-inden-1-yl)-3-formyl-4-hydroxybenzamide

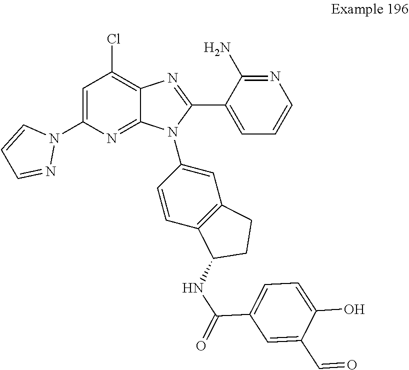 [SMILES: Nc1ncccc1-c1nc2c(Cl)cc(-n3cccn3)nc2n1-c1ccc2c(c1)CC[C@@H]2NC(=O)c1ccc(O)c(C=O)c1]

Example 196 was prepared in a manner analogous to Example 168 (Steps 3 and 4 only) using Intermediate 196-1 in place of Intermediate 33-1. MS (ESI) calculated for C₃₁H₂₃ClN₈O₃: 590.16, found 591.16 [M+H]⁺. ¹H NMR (400 MHz, DMSO-d₆) δ (ppm): 11.58 (brs, 1H), 10.32 (s, 1H), 8.94 (d, J=7.6 Hz, 1H), 8.33 (dd, J=11.0, 8.3 Hz, 2H), 8.09 (d, J=8.5 Hz, 1H), 8.03 (brs, 1H), 8.00 (d, J=6.7 Hz, 1H), 7.84 (s, 1H), 7.40 (s, 1H), 7.34 (d, J=8.9 Hz, 3H), 7.05 (dd, J=8.6, 2.8 Hz, 1H), 6.82 (s, 2H), 6.57 (s, 1H), 6.48 (dd, J=7.5, 4.4 Hz, 1H), 5.78-5.56 (m, 1H), 3.02 (dd, J=16.2, 8.9 Hz, 1H), 2.89 (dt, J=16.1, 7.9 Hz, 1H), 2.55-2.46 (m, 1H), 2.16-2.02 (m, 1H).

### Intermediate 196-1: (S)-3-(3-(1-amino-2,3-dihydro-1H-inden-5-yl)-7-chloro-5-(1H-pyrazol-1-yl)-3H-imidazo[4,5-b]pyridin-2-yl)pyridin-2-amine

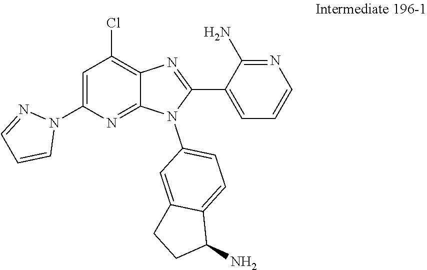 [SMILES: Nc1ncccc1-c1nc2c(Cl)cc(-n3cccn3)nc2n1-c1ccc2c(c1)CC[C@@H]2N]

### Synthetic Route:

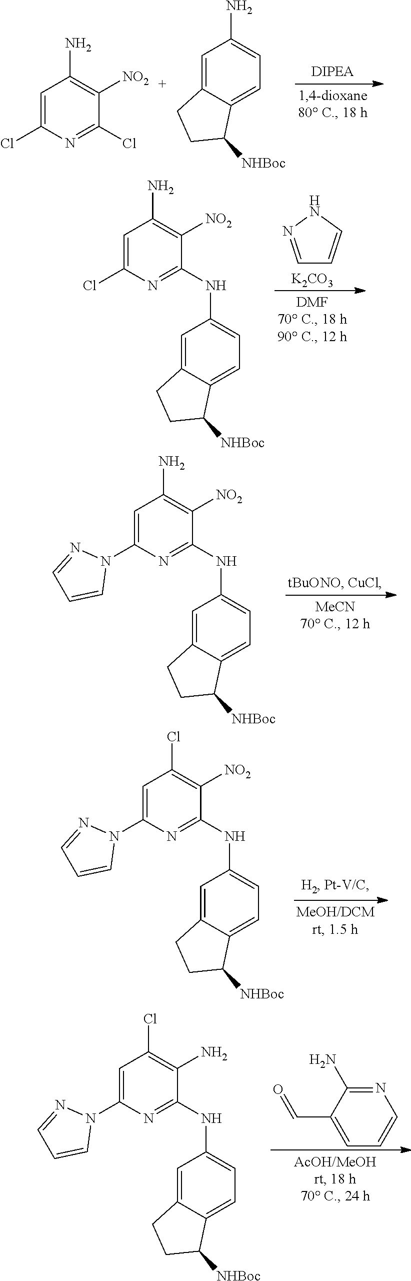

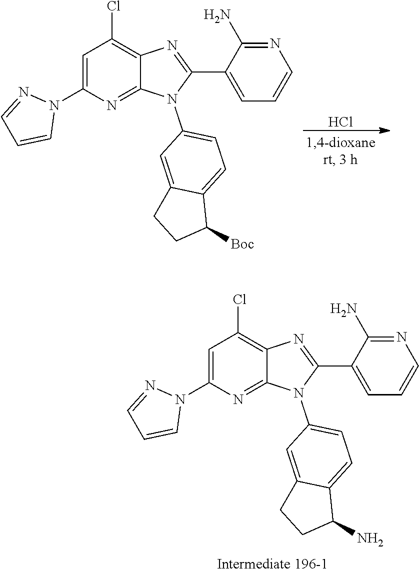 [SMILES: CC(C)(C)OC(=O)[C@H]1CCc2cc(-n3c(-c4cccnc4N)nc4c(Cl)cc(-n5cccn5)nc43)ccc21 ; Cl ; Nc1ncccc1-c1nc2c(Cl)cc(-n3cccn3)nc2n1-c1ccc2c(c1)CC[C@@H]2N]

### Step 1: Synthesis of (S)-tert-butyl (5-((4-amino-6-chloro-3-nitropyridin-2-yl)amino)-2,3-dihydro-1H-inden-1-yl)carbamate

To a solution of 2,6-dichloro-3-nitropyridin-4-amine (3.51 g, 16.4 mmol, 1.05 equiv) and (S)-tert-butyl (5-amino-2,3-dihydro-1H-inden-1-yl)carbamate (4.26 mg, 15.6 mmol, 1 equiv) in 1,4-dioxane (35 mL) was added N,N-diisopropylethylamine (8.23 mL, 46.8 mmol, 3 equiv) and the mixture was stirred at 80° C. for 18 h. The reaction mixture was concentrated in vacuo and the residue was purified by silica gel column chromatography (0-15% methanol in dichloromethane) to obtain (S)-tert-butyl (5-((4-amino-6-chloro-3-nitropyridin-2-yl)amino)-2,3-dihydro-1H-inden-1-yl)carbamate (6.25 g, 95%) as an orange solid. MS (ESI) calculated for C₁₉H₂₂ClN₅O₄: 419.14 found 420.20 [M+H]⁺.

### Step 2: Synthesis of (S)-tert-butyl (5-((4-amino-3-nitro-6-(1H-pyrazol-1-yl)pyridin-2-yl)amino)-2,3-dihydro-1H-inden-1-yl)carbamate

To a solution of (S)-tert-butyl (5-((4-amino-6-chloro-3-nitropyridin-2-yl)amino)-2,3-dihydro-1H-inden-1-yl)carbamate (4.88 g, 11.6 mmol, 1 equiv) and pyrazole (1.67 g, 23.2 mmol, 2 equiv) in N,N-dimethylformamide (23 mL) was added potassium carbonate (4.92 g, 34.9 mmol, 3 equiv) and the resulting suspension was stirred at 70° C. for 18 h and then at 90° C. for 12 h. The reaction was allowed to cool to room temperature and was diluted with diethyl ether (20 mL) followed by water (50 mL) dropwise, during which an orange solid precipitated. The solid was filtered through a Buchner funnel. The crude solid was purified by silica gel column chromatography (5-15% methanol in dichloromethane) to obtain (S)-tert-butyl (5-((4-amino-3-nitro-6-(1H-pyrazol-1-yl)pyridin-2-yl)amino)-2,3-dihydro-1H-inden-1-yl)carbamate (3.18 g, 60%) as an orange solid. MS (ESI) calculated for C₂₂H₂₅N₇O₄: 451.20, found 452.26 [M+H]⁺.

### Step 3: Synthesis of (S)-tert-butyl (5-((4-chloro-3-nitro-6-(1H-pyrazol-1-yl)pyridin-2-yl)amino)-2,3-dihydro-1H-inden-1-yl)carbamate

To a solution of (S)-tert-butyl (5-((4-amino-3-nitro-6-(1H-pyrazol-1-yl)pyridin-2-yl)amino)-2,3-dihydro-1H-inden-1-yl)carbamate (680 mg, 1.11 mmol, 1 equiv) in acetonitrile (4.1 mL) was added tert-butyl nitrite (172 mg, 1.67 mmol, 1.5 equiv) followed by copper (I) chloride (171 mg, 1.67 mmol, 1.5 equiv) and the resulting mixture was stirred at 70° C. for 12 h. The reaction mixture was concentrated and purified by silica gel column chromatography (15-20% ethyl acetate in heptanes) to obtain (S)-tert-butyl (5-((4-chloro-3-nitro-6-(1H-pyrazol-1-yl)pyridin-2-yl)amino)-2,3-dihydro-1H-inden-1-yl)carbamate (177 mg, 34%) as a yellow solid. MS (ESI) calculated for C₂₂H₂₃ClN₆O₄: 470.15, found 471.16.

### Step 4: Synthesis of (S)-tert-butyl (5-((3-amino-4-chloro-6-(1H-pyrazol-1-yl)pyridin-2-yl)amino)-2,3-dihydro-1H-inden-1-yl)carbamate

To a solution of (S)-tert-butyl (5-((4-chloro-3-nitro-6-(1H-pyrazol-1-yl)pyridin-2-yl)amino)-2,3-dihydro-1H-inden-1-yl)carbamate (87 mg, 0.18 mmol, 1 equiv) in methanol (1.8 mL) and dichloromethane (1.8 mL) was added platinum/vanadium on carbon (contains 1 wt % platinum, 2 wt % vanadium) (20 mg, 1.02 μmol, 0.5 mol %) and the mixture was stirred at room temperature for 1.5 h under hydrogen atmosphere. The reaction mixture was filtered through celite washing with dichloromethane (5 mL). The filtrate was concentrated in vacuo to obtain (S)-tert-butyl (5-((3-amino-4-chloro-6-(1H-pyrazol-1-yl)pyridin-2-yl)amino)-2,3-dihydro-1H-inden-1-yl)carbamate (79.8 mg, 100%) as a pale-yellow oil, which was used without further purification in the next step. MS (ESI) calculated for C₂₂H₂₅ClN₆O₂: 440.17, found 441.24 [M+H]⁺.

### Step 5: Synthesis of (S)-tert-butyl (5-(2-(2-aminopyridin-3-yl)-7-chloro-5-(1H-pyrazol-1-yl)-3H-imidazo[4,5-b]pyridin-3-yl)-2,3-dihydro-1H-inden-1-yl)carbamate

To a solution of (S)-tert-butyl (5-((3-amino-4-chloro-6-(1H-pyrazol-1-yl)pyridin-2-yl)amino)-2,3-dihydro-1H-inden-1-yl)carbamate (79.8 mg, 0.181 mmol, 1 equiv) in acetic acid (650 μL) and methanol (65.0 μL) was added 2-aminopyridine-3-carboxaldehyde (24.8 mg, 0.199 mmol, 1.1 equiv) and the reaction was stirred at room temperature for 18 h, then at 70° C. for 24 h. The reaction mixture was then cooled to 0° C. and quenched with 2N aqueous sodium hydroxide (until pH ˜9-10), then extracted with dichloromethane (3×5 mL). The organic layers were combined, washed with brine (3 mL), dried over sodium sulfate, and concentrated in vacuo. The residue was purified by silica gel column chromatography (60-70% ethyl acetate in heptanes) to obtain (S)-tert-butyl (5-(2-(2-aminopyridin-3-yl)-7-chloro-5-(1H-pyrazol-1-yl)-3H-imidazo[4,5-b]pyridin-3-yl)-2,3-dihydro-1H-inden-1-yl)carbamate (87 mg, 89%) as a pale-yellow solid. MS (ESI) calculated for C₂₈H₂₇ClN₈O₂: 542.19, found 543.22 [M+H]⁺.

### Step 6: Synthesis of (S)-3-(3-(1-amino-2,3-dihydro-1H-inden-5-yl)-5-(1H-pyrazol-1-yl)-3H-imidazo[4,5-b]pyridin-2-yl)pyridin-2-amine hydrochloride (Intermediate 196-1)

To a solution of (S)-tert-butyl (5-(2-(2-aminopyridin-3-yl)-7-chloro-5-(1H-pyrazol-1-yl)-3H-imidazo[4,5-b]pyridin-3-yl)-2,3-dihydro-1H-inden-1-yl)carbamate (87 mg, 0.16 mmol) in 1,4-dioxane (1.0 mL) was added a 4N solution of hydrochloric acid in 1,4-dioxane (0.8 mL) and the mixture was stirred at room temperature for 3 h. The reaction mixture was concentrated in vacuo co-evaporating with dichloromethane (3×5 mL) to obtain (S)-3-(3-(1-amino-2,3-dihydro-1H-inden-5-yl)-7-chloro-5-(1H-pyrazol-1-yl)-3H-imidazo[4,5-b]pyridin-2-yl)pyridin-2-amine (Intermediate 196-1) (76.8 mg, 100%) as a beige solid, which was used in subsequent transformations without further purification. MS (ESI) calculated for C₂₃H₁₉ClN₈: 442.14, found 443.05 [M+H]⁺.

### Example 197: N-[(1S)-5-[2-(2-aminopyridin-3-yl)-7-ethyl-5-(pyrazol-1-yl)imidazo[4,5-b]pyridin-3-yl]-2,3-dihydro-1H-inden-1-yl]-3-formyl-4-hydroxybenzamide

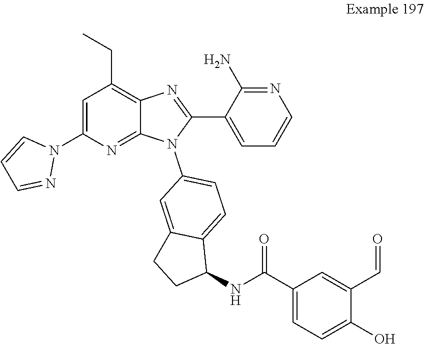 [SMILES: CCc1cc(-n2cccn2)nc2c1nc(-c1cccnc1N)n2-c1ccc2c(c1)CC[C@@H]2NC(=O)c1ccc(O)c(C=O)c1]

### Synthetic Route:

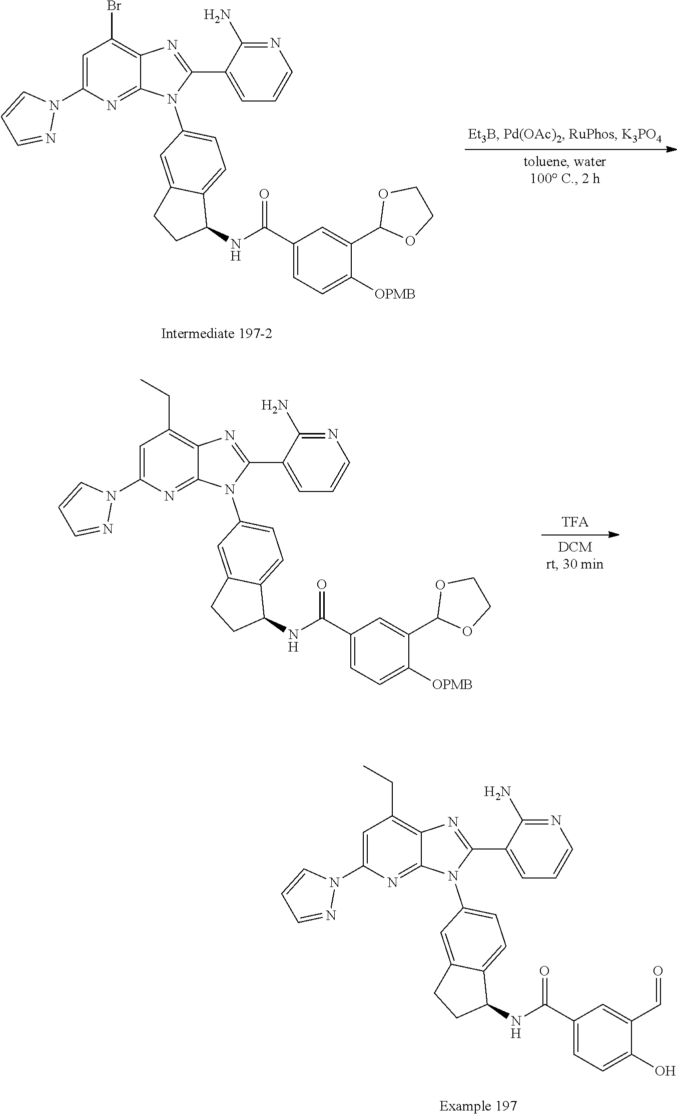

### Step 1: Synthesis of N-[(1S)-5-[2-(2-aminopyridin-3-yl)-7-ethyl-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

To a solution of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (Intermediate 197-2) (120 mg, 0.150 mmol, 1 equiv) in toluene (2.3 mL) and water (0.22 mL) was add triethylborane (7.35 mg, 0.075 mmol, 0.5 equiv), palladium (II) acetate (3.4 mg, 0.015 mmol, 0.1 equiv), RuPhos (7.0 mg, 0.015 mmol, 0.1 equiv) and tribasic potassium phosphate (63.7 mg, 0.300 mmol, 2 equiv). The mixture was stirred at 100° C. for 2 h under nitrogen atmosphere. The resulting mixture was allowed to cool to room temperature and quenched with brine. The mixture was extracted with ethyl acetate 3 times. The combined organic layers were dried over sodium sulfate, filtered and concentrated in vacuo. The resulting residue was purified by silica gel column chromatography eluting with ethyl acetate to afford N-[(1S)-5-[2-(2-aminopyridin-3-yl)-7-ethyl-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (26 mg, 23%) as an orange solid. MS (ESI) calcd. for C₄₃H₄₀N₈O₅: 748.31 m/z, found 749.30 [M+H]⁺.

### Step 2: Synthesis of N-[(1S)-5-[2-(2-aminopyridin-3-yl)-7-ethyl-5-(pyrazol-1-yl)imidazo[4,5-b]pyridin-3-yl]-2,3-dihydro-1H-inden-1-yl]-3-formyl-4-hydroxybenzamide (Example 197)

To a solution of N-[(1S)-5-[2-(2-aminopyridin-3-yl)-7-ethyl-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (26 mg, 0.035 mmol) in dichloromethane (0.75 mL) was added 2,2,2-trifluoroacetic acid (0.25 mL) and the mixture was stirred at room temperature for 30 min. The resulting mixture was concentrated in vacuo and the residue was purified by preparative HPLC on a XSelect CSH Prep C18 OBD Column using a 23-45% gradient of acetonitrile in water (+0.1% formic acid) to afford N-[(1S)-5-[2-(2-aminopyridin-3-yl)-7-ethyl-5-(pyrazol-1-yl)imidazo[4,5-b]pyridin-3-yl]-2,3-dihydro-1H-inden-1-yl]-3-formyl-4-hydroxybenzamide (Example 197) (1.0 mg, 4.8%) as an off-white solid. MS (ESI) calcd. for C₃₃H₂₈N₈O₃: 584.23 m/z, found 585.35 [M+H]⁺. ¹H NMR (300 MHz, DMSO-d₆) δ (ppm): 10.26 (s, 1H), 8.90-8.92 (m, 1H), 8.34-8.42 (m, 1H), 8.32-8.33 (m, 1H), 7.99-8.00 (m, 2H), 7.78-7.80 (m, 2H), 7.31-7.35 (m, 2H), 7.26-7.27 (m, 2H), 6.98-7.01 (m, 1H), 6.52-6.54 (m, 1H), 6.45-6.47 (m, 1H), 5.60-5.61 (m, 1H), 3.13-3.15 (m, 2H), 2.98-3.06 (m, 1H), 2.88-2.90 (m, 1H), 2.50-2.51 (m, 1H), 2.07-2.08 (m, 1H), 1.39-1.43 (m, 3H).

### Intermediate 197-2: (S)—N-(5-(2-(2-aminopyridin-3-yl)-7-bromo-5-(1H-pyrazol-1-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide

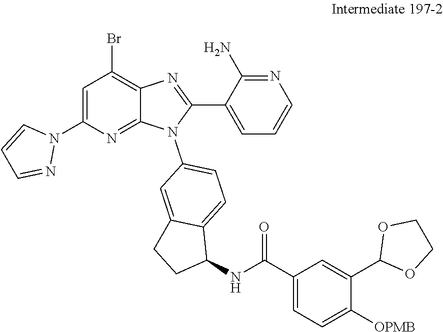

### Synthetic Route:

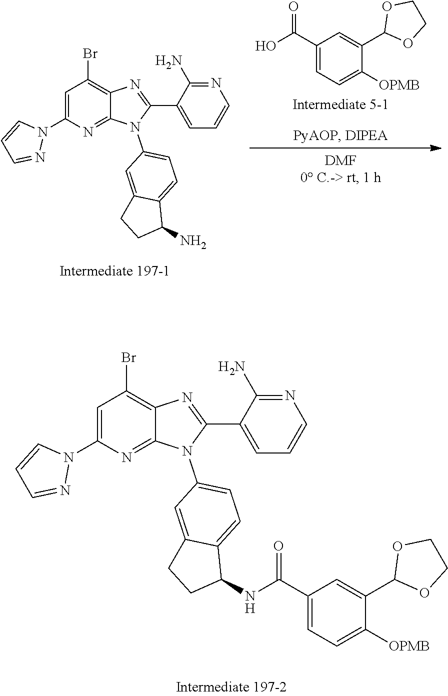

### Step 1: Synthesis of (S)—N-(5-(2-(2-aminopyridin-3-yl)-7-bromo-5-(1H-pyrazol-1-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide (Intermediate 197-2)

To a solution of (S)-3-(3-(1-amino-2,3-dihydro-1H-inden-5-yl)-7-bromo-5-(1H-pyrazol-1-yl)-3H-imidazo[4,5-b]pyridin-2-yl)pyridin-2-amine (Intermediate 197-1) (73.8 mg, 0.151 mmol, 1 equiv) and 3-(1,3-dioxolan-2-yl)-4-((4-methoxybenzyl)oxy)benzoic acid (Intermediate 5-1) (52.5 mg, 0.159 mmol, 1.05 equiv) in N,N-dimethylformamide (1 mL) was added (7-azabenzotriazol-1-yloxy)tripyrrolidinophosphonium hexafluorophosphate (PyAOP) (86.9 mg, 0.167 mmol, 1.1 equiv) and the mixture was cooled to 0° C. N,N-diisopropylethylamine (82.0 μL, 0.465 mmol, 3 equiv) was added and the resulting mixture was allowed to warm to room temperature and stirred for 1 h. The reaction mixture was then quenched with water (5 mL) and extracted with dichloromethane (4×5 mL). The organic layers were combined, washed with brine (5 mL), dried over sodium sulfate, filtered and concentrated in vacuo. The residue was purified by silica gel column chromatography (50-100% ethyl acetate in heptanes) to obtain (S)—N-(5-(2-(2-aminopyridin-3-yl)-7-bromo-5-(1H-pyrazol-1-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide (Intermediate 197-2) (98.0 mg, 81%) as a beige solid. MS (ESI) calculated for C₄₁H₃₅BrN₈O₅: 798.19, 800.19, found 799.44, 801.43 [M+H]⁺.

### Intermediate 197-1: (S)-3-(3-(1-amino-2,3-dihydro-1H-inden-5-yl)-7-bromo-5-(1H-pyrazol-1-yl)-3H-imidazo[4,5-b]pyridin-2-yl)pyridin-2-amine

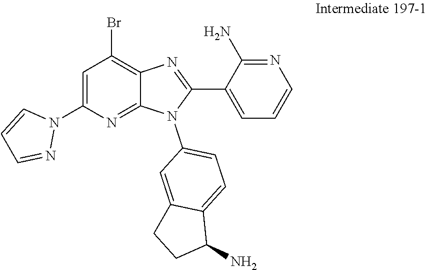 [SMILES: Nc1ncccc1-c1nc2c(Br)cc(-n3cccn3)nc2n1-c1ccc2c(c1)CC[C@@H]2N]

Intermediate 197-1 was prepared in a manner analogous to Intermediate 196-1 using copper (I) bromide in place of copper (I) chloride. MS (ESI) calculated for MS (ESI) calculated for C₂₃H₁₉BrN₈: 486.09, 488.09, found 487.11, 489.15 [M+H]⁺.

### Example 198: N-[(1S)-5-[2-(2-aminopyridin-3-yl)-5,7-bis(pyrazol-1-yl)imidazo[4,5-b]pyridin-3-yl]-2,3-dihydro-1H-inden-1-yl]-3-formyl-4-hydroxybenzamide

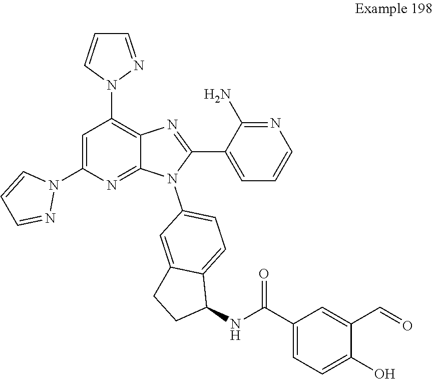 [SMILES: Nc1ncccc1-c1nc2c(-n3cccn3)cc(-n3cccn3)nc2n1-c1ccc2c(c1)CC[C@@H]2NC(=O)c1ccc(O)c(C=O)c1]

### Synthetic Route:

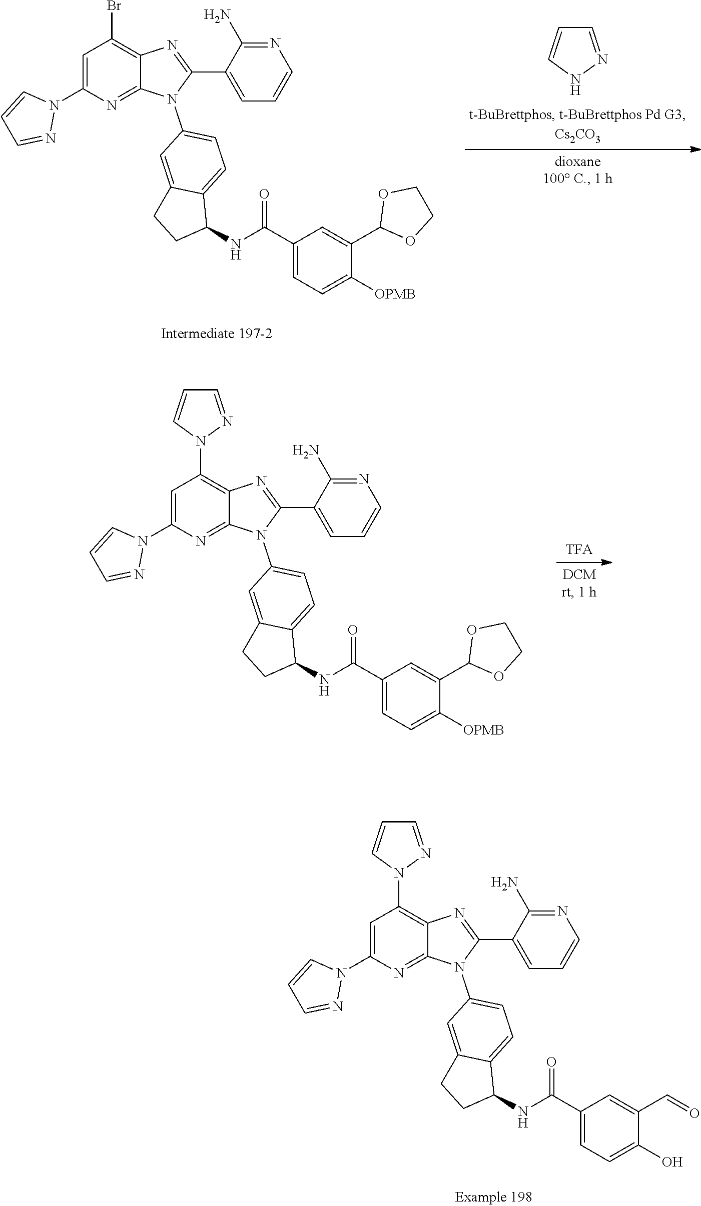

### Step 1: Synthesis of N-[(1S)-5-[2-(2-aminopyridin-3-yl)-5,7-bis(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

A suspension of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (Intermediate 197-2) (100 mg, 0.125 mmol, 1 equiv), pyrazole (26 mg, 0.38 mmol, 3 equiv), t-BuBrettPhos (13 mg, 0.025 mmol, 0.2 equiv), tBuBrettPhos Pd G3 (22 mg, 0.025 mmol, 0.2 equiv) and cesium carbonate (81 mg, 0.25 mmol, 2 equiv) in 1,4-dioxane (3 mL) was stirred at 100° C. for 1 h under nitrogen atmosphere. Brine was added and the mixture was extracted 3 times with ethyl acetate. The combined organic layers were dried over sodium sulfate, filtered and concentrated in vacuo. The resulting crude was purified by reverse-phase flash column chromatography on C18 silica gel using a 10-60% gradient of acetonitrile in water (+0.05% ammonium bicarbonate) to afford N-[(1S)-5-[2-(2-aminopyridin-3-yl)-5,7-bis(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (85 mg, 87% yield). MS (ESI) calcd. for C₄₄H₃₈N₁₀O₅: 786.30 m/z, found 787.30 [M+H]⁺.

### Step 2: Synthesis of N-[(1S)-5-[2-(2-aminopyridin-3-yl)-5,7-bis(pyrazol-1-yl)imidazo[4,5-b]pyridin-3-yl]-2,3-dihydro-1H-inden-1-yl]-3-formyl-4-hydroxybenzamide (Example 198)

To a solution of N-[(1S)-5-[2-(2-aminopyridin-3-yl)-5,7-bis(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (80 mg, 0.102 mmol) in dichloromethane (2.5 mL) was added 2,2,2-trifluoroacetic acid (0.5 mL) and the resulting mixture was stirred at room temperature for 1 h. The solution was concentrated and the crude product was purified by Preparative HPLC on a XSelect CSH Prep C18 OBD Column using a 27-49% gradient of acetonitrile in water (+0.1% formic acid) to afford N-[(1S)-5-[2-(2-aminopyridin-3-yl)-5,7-bis(pyrazol-1-yl)imidazo[4,5-b]pyridin-3-yl]-2,3-dihydro-1H-inden-1-yl]-3-formyl-4-hydroxybenzamide (Example 198) (15 mg, 24%) as a yellow solid. MS (ESI) calcd. for C₃₄H₂₆N₁₀O₃: 622.22 m/z, found 623.30 [M+H]⁺. ¹H NMR (400 MHz, DMSO-d₆) δ (ppm): 10.25 (s, 1H), 9.18-9.47 (m, 1H), 8.36-8.55 (m, 1H), 8.18-8.36 (m, 2H), 7.89-8.17 (m, 3H), 7.72-7.89 (m, 1H), 7.18-7.53 (m, 4H), 6.99-7.18 (m, 1H), 6.65-6.89 (m, 1H), 6.44-6.89 (m, 2H), 5.46-5.73 (m, 1H), 2.99-3.21 (m, 1H), 2.75-2.99 (m, 1H), 2.51-2.56 (m, 1H), 1.98-2.27 (m, 1H).

### Example 199: N-[(1S)-5-[2-(2-aminopyridin-3-yl)-5-(3-fluoropyrazol-1-yl)-7-methylimidazo[4,5-b]pyridin-3-yl]-2,3-dihydro-1H-inden-1-yl]-2,3-difluoro-5-formyl-4-hydroxybenzamide

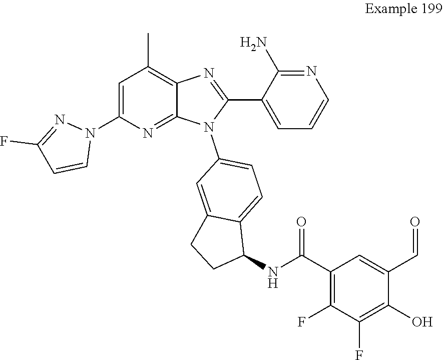 [SMILES: Cc1cc(-n2ccc(F)n2)nc2c1nc(-c1cccnc1N)n2-c1ccc2c(c1)CC[C@@H]2NC(=O)c1cc(C=O)c(O)c(F)c1F]

Example 199 was prepared in a manner analogous to Example 7 using Intermediate 199-1 in place of Intermediate 1-2 and Intermediate 118-1 in place of Intermediate 5-1. MS (ESI) calcd. for C₃₂H₂₃F₃N₈O₃: 624.18 m/z, found 625.25 [M+H]⁺. ¹H NMR (400 MHz, DMSO-d₆) δ (ppm): 10.30 (s, 1H), 8.30-8.35 (m, 1H), 8.05-8.10 (m, 1H), 7.80-7.83 (m, 1H), 7.69-7.80 (m, 1H), 7.41-7.50 (m, 1H), 7.30-7.40 (m, 2H), 7.25-7.29 (m, 1H), 6.55-6.60 (m, 1H), 6.35-6.40 (m, 1H), 5.40-5.45 (m, 1H), 2.95-3.10 (m, 2H), 2.70-2.80 (m, 3H), 2.45-2.50 (m, 1H), 1.95-2.01 (m, 1H). ¹⁹F NMR (400 MHz, DMSO-d₆) δ (ppm): −127.30, −128.35, −158.21.

### Intermediate 199-1: (S)-3-(3-(1-amino-2,3-dihydro-1H-inden-5-yl)-5-(3-fluoro-1H-pyrazol-1-yl)-7-methyl-3H-imidazo[4,5-b]pyridin-2-yl)pyridin-2-amine

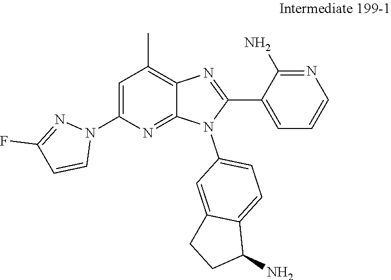 [SMILES: Cc1cc(-n2ccc(F)n2)nc2c1nc(-c1cccnc1N)n2-c1ccc2c(c1)CC[C@@H]2N]

Intermediate 199-1 was prepared in manner analogous to Intermediate 33-1 using Intermediate 131-1 in place of Intermediate 18-2, 3-fluoropyrazole in place of pyrazole, tBuBrettPhos in place of EPhos, tBuBrettPhos Pd G3 in place of EPhos Pd G4 and tribasic potassium phosphate in place of cesium carbonate. MS (ESI) calcd. for C₂₄H₂₁FN₈: 440.19 m/z, found: 441.05 [M+H]⁺.

### Example 200: N-[(1S)-5-[2-(2-aminopyridin-3-yl)-7-methoxy-5-(pyrazol-1-yl)imidazo[4,5-b]pyridin-3-yl]-2,3-dihydro-1H-inden-1-yl]-3-formyl-4-hydroxybenzamide

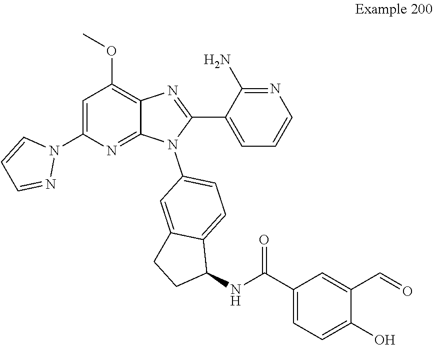 [SMILES: COc1cc(-n2cccn2)nc2c1nc(-c1cccnc1N)n2-c1ccc2c(c1)CC[C@@H]2NC(=O)c1ccc(O)c(C=O)c1]

Example 200 was prepared in a manner analogous to Example 12 using Intermediate 200-3 in place of Intermediate 1-3 and Intermediate 57-2 in place of Intermediate 12-1. MS (ESI) calcd. for C₃₂H₂₆N₈O₄: 586.21 m/z, found 587.30 [M+H]⁺. ¹H NMR (400 MHz, DMSO-d₆) δ (ppm): 10.32 (s, 1H), 8.28-8.40 (m, 2H), 8.02-8.15 (m, 2H), 7.86-7.99 (m, 2H), 7.53-7.67 (m, 2H), 7.31-7.35 (m, 2H), 7.11-7.15 (m, 1H), 6.87-6.97 (m, 1H), 6.53-6.59 (m, 1H), 5.59-5.63 (m, 1H), 4.22 (s, 3H), 3.01-3.05 (m, 1H), 2.91-2.99 (m, 1H), 2.51-2.52 (m, 1H), 2.08-2.12 (m, 1H).

### Intermediate 200-3: (S)-3-(3-(1-amino-2,3-dihydro-1H-inden-5-yl)-7-methoxy-5-(1H-pyrazol-1-yl)-3H-imidazo[4,5-b]pyridin-2-yl)pyridin-2-amine

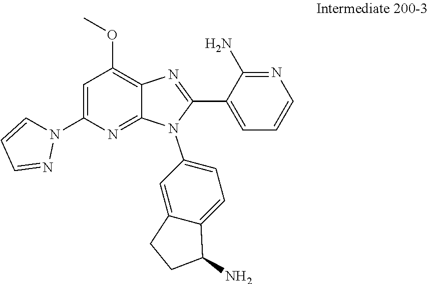 [SMILES: COc1cc(-n2cccn2)nc2c1nc(-c1cccnc1N)n2-c1ccc2c(c1)CC[C@@H]2N]

Intermediate 200-3 was prepared in a manner analogous to Intermediate 33-1 using Intermediate 200-2 in place of Intermediate 18-2. MS (ESI) calcd. for C₂₄H₂₂N₈O: 438.19 m/z, found 439.20 [M+H]⁺.

### Intermediate 200-2: (S)—N-(5-(2-(2-aminopyridin-3-yl)-5-chloro-7-methoxy-3H-imidazo[4,5-b]pyridin-3-yl)-2,3-dihydro-1H-inden-1-yl)acetamide

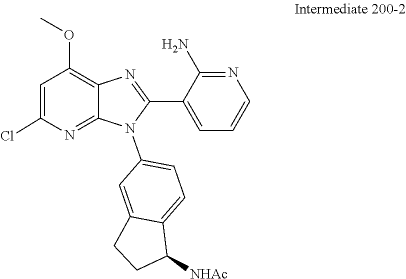 [SMILES: COc1cc(Cl)nc2c1nc(-c1cccnc1N)n2-c1ccc2c(c1)CC[C@@H]2NC(C)=O]

Intermediate 200-2 was prepared in a manner analogous to Intermediate 18-2 using Intermediate 200-1 in place of Intermediate 18-2. MS (ESI) calcd. for C₂₃H₂₁ClN₆O₂: 448.14 m/z, found 449.05 [M+H]⁺.

### Intermediate 200-1: (S)—N-(5-((6-chloro-4-methoxy-3-nitropyridin-2-yl)amino)-2,3-dihydro-1H-inden-1-yl)acetamide

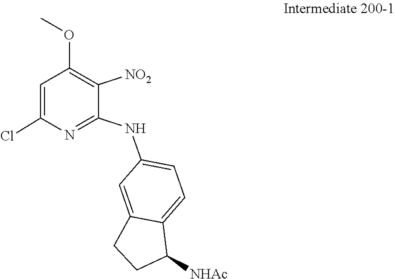 [SMILES: COc1cc(Cl)nc(Nc2ccc3c(c2)CC[C@@H]3NC(C)=O)c1[N+](=O)[O-]]

### Synthetic Route:

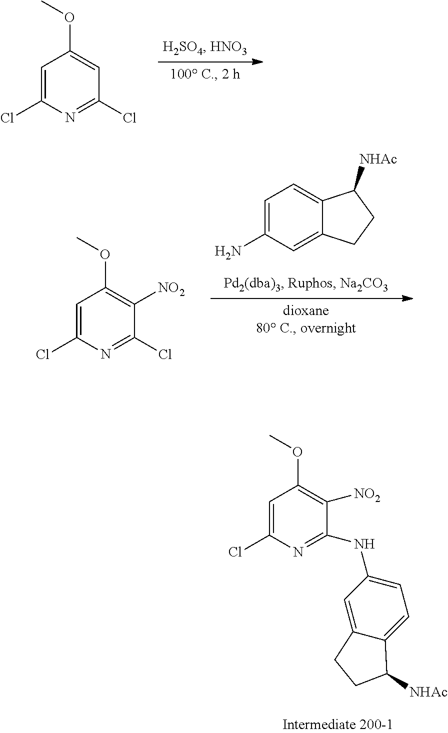

### Step 1: Synthesis of 2,6-dichloro-4-methoxy-3-nitropyridine

To a cooled (0° C.) solution of 2,6-dichloro-4-methoxypyridine (10.0 g, 56.2 mmol, 1 equiv) in sulfuric acid (60 mL) was added nitric acid (10.62 g, 168.5 mmol, 3 equiv) and the resulting mixture was stirred at 100° C. for 2 h. The reaction mixture was poured into ice water (180 mL) and the precipitated solids were collected by filtration, rinsing with water (50 mL), and dried to afford 2,6-dichloro-4-methoxy-3-nitropyridine (12 g, 86%) as a white solid.

### Step 2: Synthesis of N-[(1S)-5-[(6-chloro-4-methoxy-3-nitropyridin-2-yl)amino]-2,3-dihydro-1H-inden-1-yl]acetamide (Intermediate 200-1)

To a solution of 2,6-dichloro-4-methoxy-3-nitropyridine (4.00 g, 17.9 mmol, 1 equiv) in 1,4-dioxane (60 mL) was added N-[(1S)-5-amino-2,3-dihydro-1H-inden-1-yl]acetamide (5.12 g, 26.9 mmol, 1.5 equiv), tris(dibenzylideneacetone)dipalladium(0) (1.64 g, 1.79 mmol, 0.1 equiv), RuPhos (1.674 g, 3.587 mmol, 0.2 equiv) and sodium carbonate (5.703 g, 53.81 mmol, 3 equiv). The resulting mixture was stirred overnight at 80° C. under nitrogen atmosphere. The reaction mixture was cooled to room temperature and treated with water (60 mL), extracted with ethyl acetate (60 mL×3). The combined extracts were washed with brine (60 mL), dried over anhydrous sodium sulfate, filtered, and concentrated to dryness in vacuo. The crude was then purified by silica gel column chromatography using a 0-10% gradient of methanol in dichloromethane to afford N-[(1S)-5-[(6-chloro-4-methoxy-3-nitropyridin-2-yl)amino]-2,3-dihydro-1H-inden-1-yl]acetamide (Intermediate 200-1) (1.2 g, 16%) as a yellow solid. MS (ESI) calcd. for C₁₇H₁₇ClN₄O₄: 376.09 m/z, found 375.00 [M−H]⁻.

### Example 201: N-[(1S)-5-[2-(2-aminopyridin-3-yl)-7-ethynyl-5-(pyrazol-1-yl)imidazo[4,5-b]pyridin-3-yl]-2,3-dihydro-1H-inden-1-yl]-3-formyl-4-hydroxybenzamide

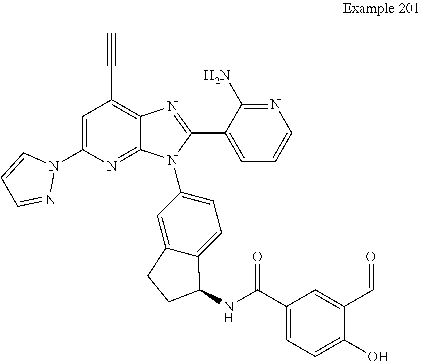 [SMILES: C#Cc1cc(-n2cccn2)nc2c1nc(-c1cccnc1N)n2-c1ccc2c(c1)CC[C@@H]2NC(=O)c1ccc(O)c(C=O)c1]

### Synthetic Route:

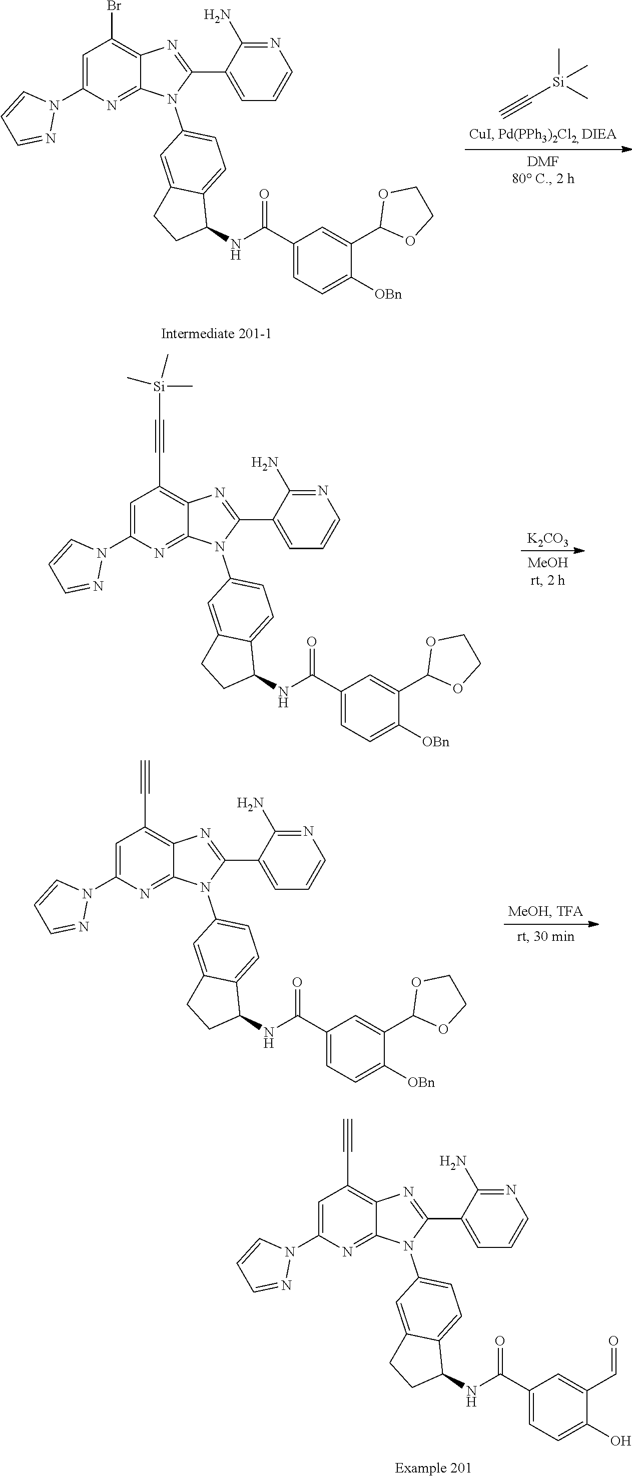

### Step 1: Synthesis of N-[(1S)-5-[2-(2-aminopyridin-3-yl)-5-(pyrazol-1-yl)-7-[2-(trimethylsilyl)ethynyl]imidazo[4,5-b]pyridin-3-yl]-2,3-dihydro-1H-inden-1-yl]-4-(benzyloxy)-3-(1,3-dioxolan-2-yl)benzamide

To a solution of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (Intermediate 201-1) (185 mg, 0.240 mmol, 1 equiv) in N,N-dimethylformamide (3 mL) was add N,N-diisopropylethylamine (62 mg, 0.48 mmol, 2 equiv), copper (I) iodide (2.3 mg, 0.012 mmol, 0.05 equiv), bis(triphenylphosphine)palladium(II) dichloride (8.4 mg, 0.012 mmol, 0.05 equiv) and trimethylsilylacetylene (236 mg, 2.40 mmol, 10 equiv). The resulting mixture was stirred at 80° C. under nitrogen atmosphere for 2 h. The reaction was quenched with water (50 mL) and extracted with ethyl acetate (3×50 mL). The combined organic layers were dried over sodium sulfate, filtered, and concentrated in vacuo. The resulting crude material was purified by reverse-phase flash column chromatography on C18 silica gel using a 5-55% gradient of acetonitrile in water (+0.05% 2,2,2-trifluoroacetic acid) to afford N-[(1S)-5-[2-(2-aminopyridin-3-yl)-5-(pyrazol-1-yl)-7-[2-(trimethylsilyl)ethynyl]imidazo[4,5-b]pyridin-3-yl]-2,3-dihydro-1H-inden-1-yl]-4-(benzyloxy)-3-(1,3-dioxolan-2-yl)benzamide (90 mg, 48%) as an orange solid. MS (ESI) calcd. for C₄₅H₄₂N₈O₄Si: 786.31 m/z, found 787.35 [M+H]⁺.

### Step 2: Synthesis of N-[(1S)-5-[2-(2-aminopyridin-3-yl)-7-ethynyl-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide

To a solution of N-[(1S)-5-[2-(2-aminopyridin-3-yl)-5-(pyrazol-1-yl)-7-[2-(trimethylsilyl)ethynyl]imidazo[4,5-b]pyridin-3-yl]-2,3-dihydro-1H-inden-1-yl]-4-(benzyloxy)-3-(1,3-dioxolan-2-yl)benzamide (90 mg, 0.114 mmol, 1 equiv) in methanol (2 mL) was add potassium carbonate (47 mg, 0.34 mmol, 3 equiv) and the mixture was stirred for 2 h at room temperature. The reaction was quenched with water (50 mL) and extracted with ethyl acetate (3×50 mL). The combined organic layers were dried over sodium sulfate, filtered, and concentrated in vacuo. The resulting crude material was purified by reverse-phase flash chromatography on C18 silica gel using a 5-75% gradient of acetonitrile in water (+0.05% 2,2,2-trifluoroacetic acid) to afford N-[(1S)-5-[2-(2-aminopyridin-3-yl)-7-ethynyl-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (64 mg, 78%) as an orange solid. MS (ESI) calcd. for C₄₂H₃₄N₈O₄: 714.27 m/z, found 715.25 [M+H]⁺.

### Step 3: Synthesis of N-[(1S)-5-[2-(2-aminopyridin-3-yl)-7-ethynyl-5-(pyrazol-1-yl)imidazo[4,5-b]pyridin-3-yl]-2,3-dihydro-1H-inden-1-yl]-3-formyl-4-hydroxybenzamide (Example 201)

To a solution of N-[(1S)-5-[2-(2-aminopyridin-3-yl)-7-ethynyl-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (64 mg, 0.090 mmol) in 2,2,2-trifluoroacetic acid (0.5 mL) was added methanesulfonic acid (2.5 mL). The mixture was stirred at room temperature for 30 min. The reaction was quenched with water (50 mL) and extracted with ethyl acetate (3×50 mL). The combined organic layers were dried over sodium sulfate, filtered, and concentrated in vacuo. The crude material was purified by preparative HPLC on a XBridge Prep OBD C18 Column using a 25-45% gradient of acetonitrile in water (+10 mmol/L ammonium bicarbonate) to afford N-[(1S)-5-[2-(2-aminopyridin-3-yl)-7-ethynyl-5-(pyrazol-1-yl)imidazo[4,5-b]pyridin-3-yl]-2,3-dihydro-1H-inden-1-yl]-3-formyl-4-hydroxybenzamide (Example 201) (15.2 mg, 28%) as a light yellow solid. MS (ESI) calcd. for C₃₃H₂₄N₈O₃: 580.20 m/z, found 581.30 [M+H]⁺. ¹H NMR (300 MHz, DMSO-d₆) δ (ppm): 10.25 (s, 1H), 8.33-8.37 (m, 2H), 7.93-8.03 (m, 4H), 7.82-7.88 (m, 1H), 7.57-7.62 (m, 1H), 7.31-7.40 (m, 4H), 6.87-7.04 (m, 1H), 6.46-6.56 (m, 2H), 5.62-5.64 (m, 1H), 4.97-4.98 (m, 1H), 2.88-3.02 (m, 2H), 2.50-2.51 (m, 1H), 2.08-2.11 (m, 1H).

### Intermediate 201-1: (S)—N-(5-(2-(2-aminopyridin-3-yl)-7-bromo-5-(1H-pyrazol-1-yl)-3H-imidazo[4,5-b]pyridin-3-yl)-2,3-dihydro-1H-inden-1-yl)-4-(benzyloxy)-3-(1,3-dioxolan-2-yl)benzamide

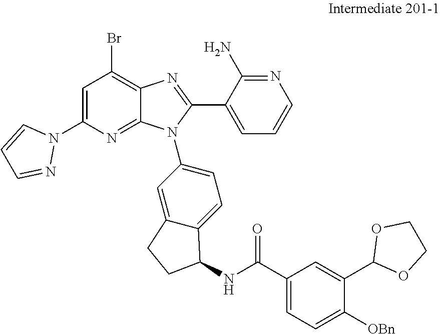 [SMILES: Nc1ncccc1-c1nc2c(Br)cc(-n3cccn3)nc2n1-c1ccc2c(c1)CC[C@@H]2NC(=O)c1ccc(OCc2ccccc2)c(C2OCCO2)c1]

Intermediate 201-1 was prepared in a manner analogous to Intermediate 197-2 using Intermediate 57-2 in place of Intermediate 5-1 and PyBOP in in place of PyAOP. MS (ESI) calcd. for C₄₀H₃₃BrN₈O₄: 768.18 m/z, found 769.25 [M+H]⁺.

### Example 202: (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-2-fluoro-5-formyl-4-hydroxybenzamide

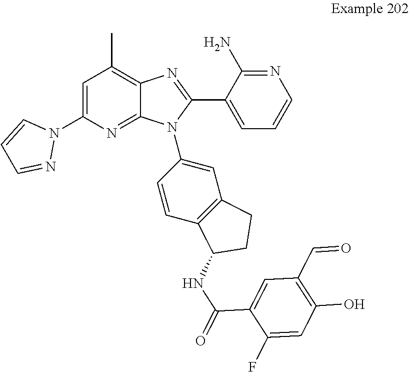 [SMILES: Cc1cc(-n2cccn2)nc2c1nc(-c1cccnc1N)n2-c1ccc2c(c1)CC[C@@H]2NC(=O)c1cc(C=O)c(O)cc1F]

Example 202 was prepared in a manner analogous to Example 7 using Intermediate 131-2 in place of Intermediate 1-2 and Intermediate 16-1 in place of Intermediate 5-1. MS (ESI) calcd. for C₃₂H₂₅FN₈O₃: 588.20 m/z, found 589.15 [M+H]⁺. ¹H NMR (400 MHz, DMSO-d₆) δ (ppm): 10.18 (m, 1H), 8.69-8.80 (m, 1H), 8.31-8.35 (m, 1H), 8.00-8.12 (m, 2H), 7.75-7.82 (m, 2H), 7.38-7.42 (m, 2H), 7.25-7.38 (m, 2H), 6.70-6.82 (m, 1H), 6.55-6.58 (m, 1H), 6.45-6.52 (m, 1H), 5.45-5.58 (m, 1H), 2.91-3.01 (m, 1H), 2.81-2.90 (m, 1H), 2.72 (s, 3H), 2.52-2.54 (m, 1H), 1.91-2.08 (m, 1H). ¹⁹F NMR (400 MHz, DMSO-d₆) δ (ppm): −102.11.

### Example 203: N-[(1S)-5-[2-(2-aminopyridin-3-yl)-5-(5-methyl-1,3,4-thiadiazol-2-yl)imidazo[4,5-b]pyridin-3-yl]-2,3-dihydro-1H-inden-1-yl]-3-formyl-4-hydroxybenzamide

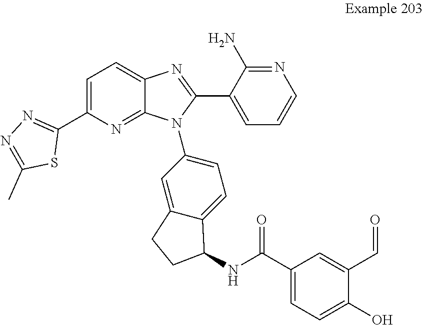 [SMILES: Cc1nnc(-c2ccc3nc(-c4cccnc4N)n(-c4ccc5c(c4)CC[C@@H]5NC(=O)c4ccc(O)c(C=O)c4)c3n2)s1]

Example 203 was prepared in a manner analogous to Example 7 using Intermediate 203-2 in place of Intermediate 1-2 and Intermediate 57-2 in place of Intermediate 5-1. MS (ESI) calcd. for C₃₁H₂₄N₈O₃S: 588.17 m/z, found 589.25 [M+H]⁺. ¹H NMR (400 MHz, DMSO-d₆) δ (ppm): 10.10-10.40 (m, 1H), 8.30-8.50 (m, 1H), 8.20-8.30 (m, 2H), 7.90-8.10 (m, 2H), 7.20-7.45 (m, 4H), 6.85-7.10 (m, 1H), 6.35-6.55 (m, 1H), 5.50-5.78 (m, 1H), 2.85-3.15 (m, 2H), 2.70-2.80 (m, 3H), 2.50-2.60 (m, 1H), 1.95-2.20 (m, 1H).

### Intermediate 203-2: (S)-3-(3-(1-amino-2,3-dihydro-1H-inden-5-yl)-5-(5-methyl-1,3,4-thiadiazol-2-yl)-3H-imidazo[4,5-b]pyridin-2-yl)pyridin-2-amine

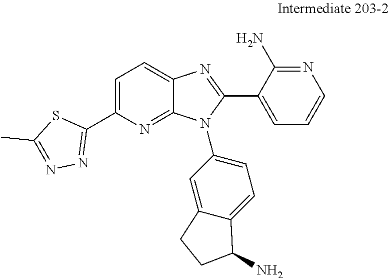 [SMILES: Cc1nnc(-c2ccc3nc(-c4cccnc4N)n(-c4ccc5c(c4)CC[C@@H]5N)c3n2)s1]

### Synthetic Route:

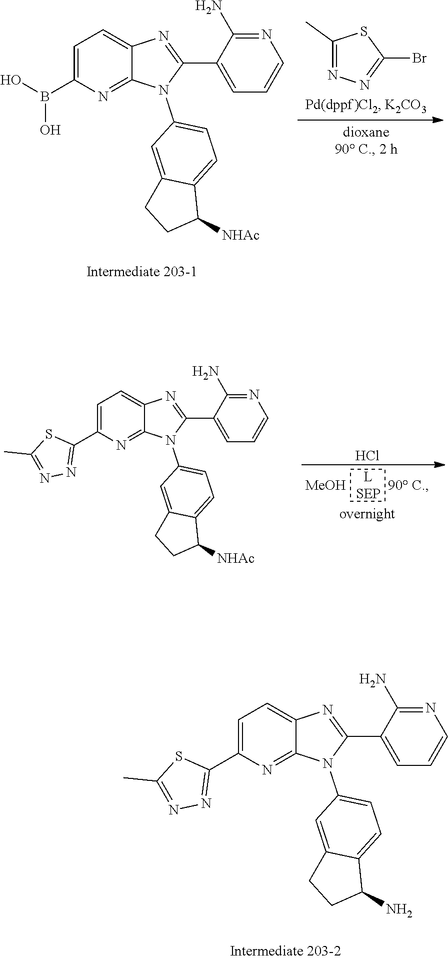

### Step 1: Synthesis of (S)—N-(5-(2-(2-aminopyridin-3-yl)-5-(5-methyl-1,3,4-thiadiazol-2-yl)-3H-imidazo[4,5-b]pyridin-3-yl)-2,3-dihydro-1H-inden-1-yl)acetamide

To a solution of 2-(2-aminopyridin-3-yl)-3-[(1S)-1-acetamido-2,3-dihydro-1H-inden-5-yl]imidazo[4,5-b]pyridin-5-ylboronic acid (Intermediate 203-1) (300 mg, 0.701 mmol, 1 equiv), 2-bromo-5-methyl-1,3,4-thiadiazole (163 mg, 0.911 mmol, 1.3 equiv) in 1,4-dioxane (6 mL) and water (1.5 mL) were added [1,1′-bis(diphenylphosphino)ferrocene]dichloropalladium(II) (51 mg, 0.070 mmol, 0.1 equiv) and potassium carbonate (193 mg, 1.4 mmol, 2 equiv). The resulting mixture was stirred at 90° C. for 2 h under nitrogen atmosphere. The mixture was allowed to cool down to room temperature and the reaction was quenched with water (100 mL). The resulting mixture was extracted with ethyl acetate (2×100 mL). The combined organic extracts were washed with brine (100 mL), dried over anhydrous sodium sulfate, filtered, and concentrated. The crude product was purified by reverse-phase flash column chromatography on C18 silica gel using a 10-60% gradient of acetonitrile in water (+0.05% ammonium bicarbonate) to afford N-[(1S)-5-[2-(2-aminopyridin-3-yl)-5-(5-methyl-1,3,4-thiadiazol-2-yl)imidazo[4,5-b]pyridin-3-yl]-2,3-dihydro-1H-inden-1-yl]acetamide (240 mg, 62%) as a brown solid. MS (ESI) calcd. for C₂₅H₂₂N₈OS: 482.16 m/z, found 483.10 [M+H]⁺.

### Step 2: Synthesis of 3-{3-[(1S)-1-amino-2,3-dihydro-1H-inden-5-yl]-5-(5-methyl-1,3,4-thiadiazol-2-yl)imidazo[4,5-b]pyridin-2-yl}pyridin-2-amine (Intermediate 203-2)

To a solution of N-[(1S)-5-[2-(2-aminopyridin-3-yl)-5-(1,3,4-thiadiazol-2-yl)imidazo[4,5-b]pyridin-3-yl]-2,3-dihydro-1H-inden-1-yl]acetamide (200 mg, 0.427 mmol) in methanol (20 mL) was added hydrochloric acid (30 mL, concentrated) and the resulting solution was stirred at 90° C. overnight. The resulting mixture was concentrated under reduced pressure to afford 3-{3-[(1S)-1-amino-2,3-dihydro-1H-inden-5-yl]-5-(5-methyl-1,3,4-thiadiazol-2-yl)imidazo[4,5-b]pyridin-2-yl}pyridin-2-amine (Intermediate 203-2) (211 mg, 67%) as a yellow solid. MS (ESI) calcd. for C₂₃H₂₀N₈S: 440.15 m/z, found 441.10 [M+H]⁺.

### Intermediate 203-1: (S)-(3-(1-acetamido-2,3-dihydro-1H-inden-5-yl)-2-(2-aminopyridin-3-yl)-3H-imidazo[4,5-b]pyridin-5-yl)boronic acid

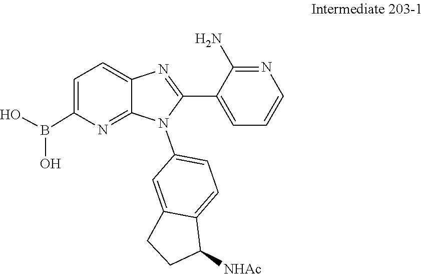 [SMILES: CC(=O)N[C@H]1CCc2cc(-n3c(-c4cccnc4N)nc4ccc(B(O)O)nc43)ccc21]

### Synthetic Route:

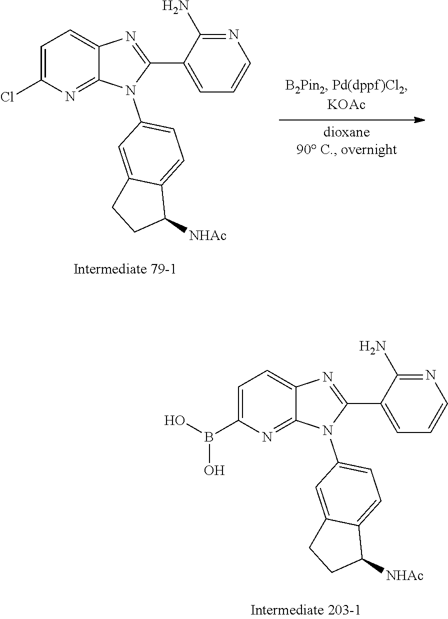 [SMILES: CC(=O)N[C@H]1CCc2cc(-n3c(-c4cccnc4N)nc4ccc(Cl)nc43)ccc21 ; CC(=O)N[C@H]1CCc2cc(-n3c(-c4cccnc4N)nc4ccc(B(O)O)nc43)ccc21]

### Step 1: Synthesis of (S)-(3-(1-acetamido-2,3-dihydro-1H-inden-5-yl)-2-(2-aminopyridin-3-yl)-3H-imidazo[4,5-b]pyridin-5-yl)boronic acid (Intermediate 203-1)

(S)—N-(5-(2-(2-aminopyridin-3-yl)-5-chloro-3H-imidazo[4,5-b]pyridin-3-yl)-2,3-dihydro-1H-inden-1-yl)acetamide (Intermediate 79-1) (500 mg, 1.19 nmol, 1 equiv) and 4,4,4′,4′,5,5,5′,5′-octamethyl-2,2′-bi(1,3,2-dioxaborolane) (1.516 mg, 5.966 mmol, 5 equiv) were dissolved in 1,4-dioxane (8 mL). [1,1′-bis(diphenylphosphino)ferrocene]dichloropalladium(II)-dichloromethane complex (96.5 mg, 0.119 mmol, 0.1 equiv) and potassium acetate (350 mg, 3.58 mmol, 3 equiv) were added and the mixture was stirred overnight at 90° C. under nitrogen atmosphere. Water (100 mL) was added and the mixture was extracted with ethyl acetate (100 mL×3). The combined organic layers were washed with brine (100 mL), dried over anhydrous sodium sulfate, filtered, and concentrated under reduced pressure. The residue was purified by silica gel column chromatography using a gradient of methanol in dichloromethane to afford (S)-(3-(1-acetamido-2,3-dihydro-1H-inden-5-yl)-2-(2-aminopyridin-3-yl)-3H-imidazo[4,5-b]pyridin-5-yl)boronic acid (Intermediate 203-1) (350 mg, 68%) as a yellow solid. MS (ESI) calcd. for C₂₂H₂₁BN₆O₃: 428.18 m/z, found 429.20 [M+H]⁺.

### Example 204: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2,3-difluoro-5-(formyl-d)-4-hydroxybenzamide

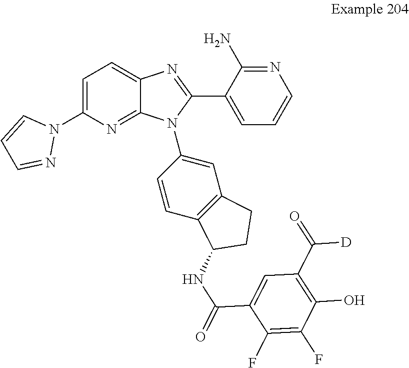 [SMILES: [2H]C(=O)c1cc(C(=O)N[C@H]2CCc3cc(-n4c(-c5cccnc5N)nc5ccc(-n6cccn6)nc54)ccc32)c(F)c(F)c1O]

Example 204 was prepared in a manner analogous to Example 164 starting from Example 82 instead of Example 33. MS (ESI) calcd. for C₃₁H₂₁DF₂N₈O₃, 593.18 m/z, found 594.15 [M+H]⁺. ¹H NMR (400 MHz, DMSO-d₆) δ (ppm): 8.25-8.40 (m, 2H), 7.90-8.15 (m, 2H), 7.75-7.82 (m, 2H), 7.38-7.45 (m, 2H), 7.22-7.38 (m, 2H), 6.50-6.58 (m, 1H), 6.35-6.50 (m, 1H), 5.45-5.52 (m, 1H), 2.95-3.11 (m, 1H), 2.81-2.95 (m, 1H), 2.52-2.55 (m, 1H), 1.90-2.11 (m, 1H). ¹⁹F NMR (400 MHz, DMSO-d₆) δ (ppm): −129.24, −159.00.

### Example 205: N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-2-(pyrazol-1-yl)benzamide

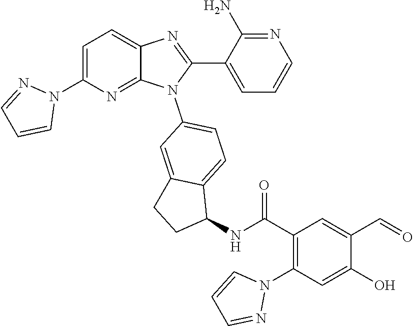 [SMILES: Nc1ncccc1-c1nc2ccc(-n3cccn3)nc2n1-c1ccc2c(c1)CC[C@@H]2NC(=O)c1cc(C=O)c(O)cc1-n1cccn1]

### Synthetic Route:

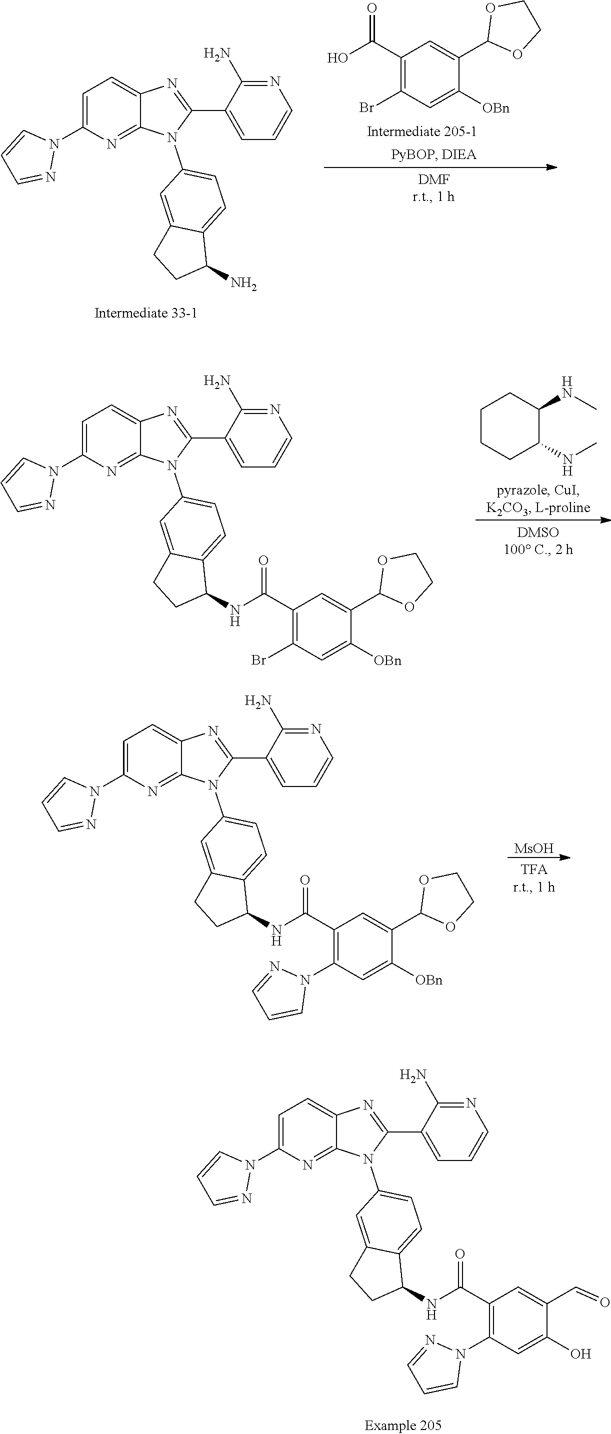

### Step 1: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-2-bromo-5-(1,3-dioxolan-2-yl)benzamide

To a solution of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (134 mg, 0.328 mmol, 1 equiv) in N,N-dimethylformamide (3 mL) was added N,N-diisopropylethylamine (127 mg, 0.984 mmol, 3 equiv), PyBOP (205 mg, 0.394 mmol, 1.2 equiv), 4-(benzyloxy)-2-bromo-5-(1,3-dioxolan-2-yl)benzoic acid (Intermediate 205-1) (124 mg, 0.328 mmol, 1 equiv). The resulting mixture was stirred at room temperature for 1 h. The crude mixture was purified by reverse-phase flash column chromatography on C18 silica gel using a 5-50% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-2-bromo-5-(1,3-dioxolan-2-yl)benzamide (92 mg, 36) as a yellow solid. MS (ESI) calcd. for C₄₀H₃₃BrN₈O₄: 768.18 m/z, found 769.10 [M+H]⁺.

### Step 2: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-(pyrazol-1-yl)benzamide

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-2-bromo-5-(1,3-dioxolan-2-yl)benzamide (50 mg, 0.065 mmol, 1 equiv) in DMSO (3 mL) was added pyrazole (4.4 mg, 0.065 mmol, 1 equiv), (1R,2R)—N1,N2-dimethylcyclohexane-1,2-diamine (0.46 mg, 0.003 mmol, 0.05 equiv), copper (I) iodide (1.9 mg, 0.010 mmol, 0.15 equiv), potassium carbonate (22 mg, 0.16 mmol, 2.5 equiv) and L-proline (2.24 mg, 0.019 mmol, 0.3 equiv). The resulting mixture was stirred at 100° C. for 2 h under nitrogen atmosphere. The crude mixture was purified by reverse-phase flash column chromatography on C18 silica gel using a 5-55% gradient of acetonitrile in water (+0.05% ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-(pyrazol-1-yl)benzamide (23 mg, 50%) as a white solid. MS (ESI) calcd. for C₄₃H₃₆N₁₀O₄: 756.29 m/z, found 757.30 [M+H]⁺.

### Step 3: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-2-(pyrazol-1-yl)benzamide (Example 205)

To a stirred solution of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(1,3-dioxolan-2-yl)-2-(pyrazol-1-yl)benzamide (23 mg, 0.030 mmol) in 2,2,2-trifluoroacetic acid (1.8 mL) was added methanesulfonic acid (0.6 mL) and the resulting mixture was stirred at room temperature for 1 h. The mixture was neutralized to pH 7 with saturated aqueous sodium bicarbonate and the resulting mixture was extracted with ethyl acetate (3×30 mL). The combined organic layers were dried over anhydrous sodium sulfate, filtered and concentrated under reduced pressure. The crude residue was purified by preparative HPLC on a XBridge Prep OBD C18 Column using a 27-50% gradient of acetonitrile in water (+10 mmol/L ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-2-(pyrazol-1-yl)benzamide (4.6 mg, 24% yield) as a yellow solid. MS (ESI) calcd. for C₃₄H₂₆N₁₀O₃: 622.22 m/z, found 623.30 [M+H]⁺. ¹H-NMR (400 MHz, DMSO-d₆) δ (ppm): 10.20 (s, 1H), 8.30-8.39 (m, 2H), 7.89-8.05 (m, 3H), 7.75-7.88 (m, 2H), 7.67-7.74 (m, 1H), 7.43-7.55 (m, 1H), 7.22-7.35 (m, 3H), 6.90-7.05 (m, 1H), 6.45-6.61 (m, 3H), 5.35-5.45 (m, 1H), 2.80-3.05 (m, 2H), 2.41-2.52 (m, 1H), 1.85-2.05 (m, 1H).

### Intermediate 205-1: 4-(benzyloxy)-2-bromo-5-(1,3-dioxolan-2-yl)benzoic acid

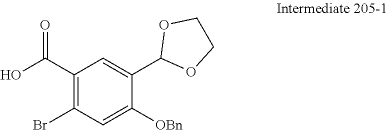 [SMILES: O=C(O)c1cc(C2OCCO2)c(OCc2ccccc2)cc1Br]

Intermediate 205-1 was prepared in a manner analogous to Intermediate 93-1 using methyl 2-bromo-4-hydroxybenzoate in place of 3-chloro-4-hydroxybenzoic acid. MS (ESI) calcd. for C₁₇H₁₅BrO₅: 378.01 m/z, found 379.00 [M+H]⁺.

### Example 206: N-[(1S)-5-[2-(2-aminopyridin-3-yl)-5-{4H,6H-furo[3,4-c]pyrazol-1-yl}imidazo[4,5-b]pyridin-3-yl]-2,3-dihydro-1H-inden-1-yl]-3-formyl-4-hydroxybenzamide

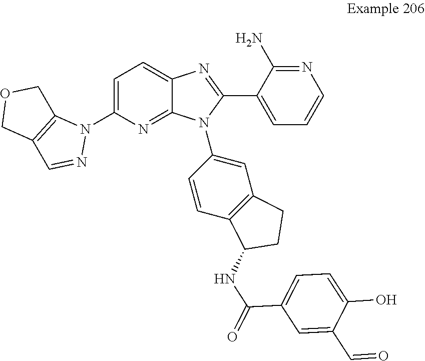 [SMILES: Nc1ncccc1-c1nc2ccc(-n3ncc4c3COC4)nc2n1-c1ccc2c(c1)CC[C@@H]2NC(=O)c1ccc(O)c(C=O)c1]

Example 206 was prepared in a manner analogous to Example 94-1 using 2H,4H,6H-furo[3,4-c]pyrazole in place of 4-(difluoromethyl)-1H-pyrazole, Intermediate 165-1 in place of Intermediate 94-1 and 2,2,2-trifluoroacetic acid/dichloromethane (4:1) instead of 2,2,2-trifluoroacetic acid/methanesulfonic acid. Two regioisomers were obtained from the first step and were separated before deprotection to provide the regioisomeric Examples 206 and 207 (vide infra), which were characterized by ¹H NMR. MS (ESI) calcd. for C₃₃H₂₆N₈O₄: 598.21 m/z, found 599.15 [M+H]⁺. ¹H NMR (400 MHz, DMSO-d₆) δ (ppm): 10.17 (s, 1H), 8.29-8.31 (m, 1H), 8.11-8.12 (m, 2H), 7.96-7.98 (m, 1H), 7.86-7.90 (m, 2H), 7.29-7.31 (m, 4H), 7.80-7.82 (m, 1H), 6.46-6.49 (m, 1H), 5.53-5.57 (m, 1H), 4.78-4.79 (m, 4H), 2.89-3.00 (m, 1H), 2.87-2.89 (m, 1H), 2.50-2.54 (m, 1H), 2.10-2.11 (m, 1H).

### Example 207: N-[(1S)-5-[2-(2-aminopyridin-3-yl)-5-{4H,6H-furo[3,4-c]pyrazol-2-yl}imidazo[4,5-b]pyridin-3-yl]-2,3-dihydro-1H-inden-1-yl]-3-formyl-4-hydroxybenzamide

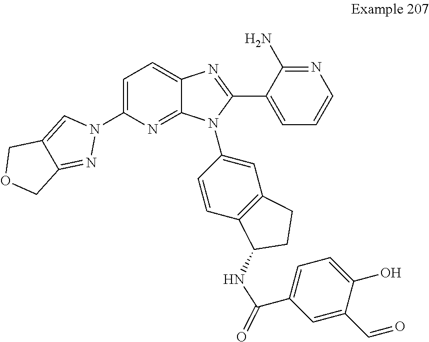 [SMILES: Nc1ncccc1-c1nc2ccc(-n3cc4c(n3)COC4)nc2n1-c1ccc2c(c1)CC[C@@H]2NC(=O)c1ccc(O)c(C=O)c1]

Example 207 was prepared in a manner analogous to Example 94-1 using 2H,4H,6H-furo[3,4-c]pyrazole in place of 4-(difluoromethyl)-1H-pyrazole, Intermediate 165-1 in place of Intermediate 94-1 and 2,2,2-trifluoroacetic acid/dichloromethane (4:1) instead of 2,2,2-trifluoroacetic acid/methanesulfonic acid. Two regioisomers were obtained from the first step and were separated before deprotection to provide the regioisomeric Examples 206 (vide supra) and 207, which were characterized by ¹H NMR. MS (ESI) calcd. for C₃₃H₂₆N₈O₄: 598.21 m/z, found 599.15 [M+H]⁺. ¹H NMR (400 MHz, DMSO-d₆) δ (ppm): 10.22 (s, 1H), 8.33-8.35 (m, 1H), 8.01-8.02 (m, 1H), 8.00-8.01 (m, 1H), 7.85-7.87 (m, 2H), 7.56-7.57 (m, 1H), 7.43-7.44 (m, 1H), 7.29-7.31 (m, 2H), 7.13-7.15 (m, 1H), 6.89-6.90 (m, 1H), 6.45-6.48 (m, 1H), 5.59-5.60 (m, 1H), 4.89-4.91 (m, 2H), 4.79-4.80 (m, 2H), 3.00-3.01 (m, 1H), 2.97-2.99 (m, 1H), 2.50-2.51 (m, 1H), 2.10-2.11 (m, 1H).

### Example 208: (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-hydroxy-3-oxoisoindoline-5-carbaldehyde

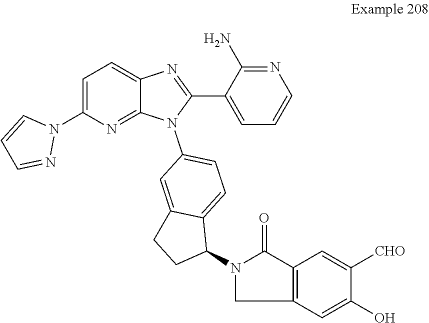 [SMILES: Nc1ncccc1-c1nc2ccc(-n3cccn3)nc2n1-c1ccc2c(c1)CC[C@@H]2N1Cc2cc(O)c(C=O)cc2C1=O]

### Synthetic Route:

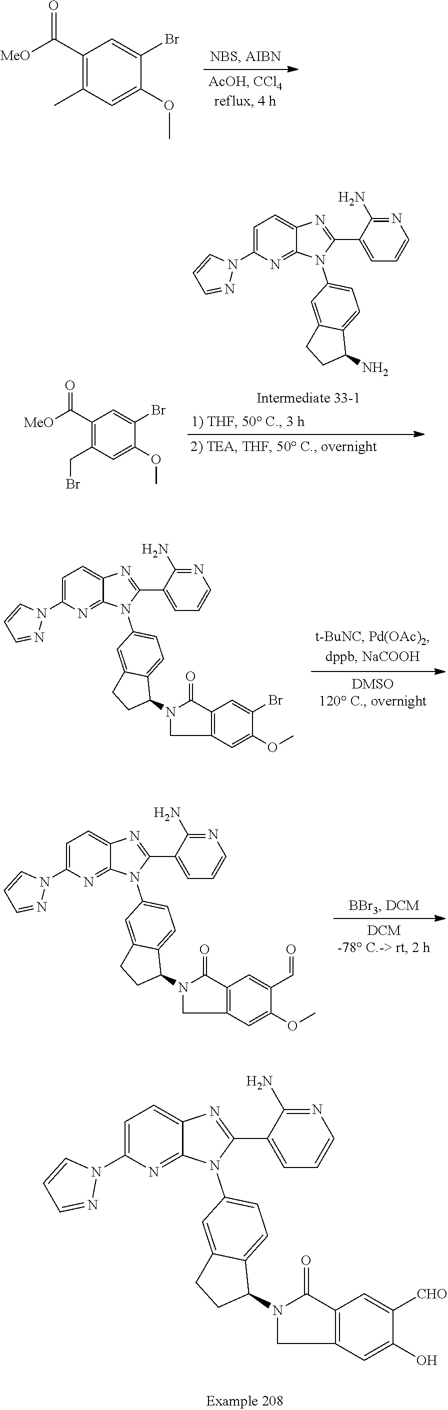 [SMILES: COC(=O)c1cc(Br)c(OC)cc1C ; Nc1ncccc1-c1nc2ccc(-n3cccn3)nc2n1-c1ccc2c(c1)CC[C@@H]2N ; COC(=O)c1cc(Br)c(OC)cc1CBr ; COc1cc2c(cc1Br)C(=O)N([C@H]1CCc3cc(-n4c(-c5cccnc5N)nc5ccc(-n6cccn6)nc54)ccc31)C2 ; COc1cc2c(cc1C=O)C(=O)N([C@H]1CCc3cc(-n4c(-c5cccnc5N)nc5ccc(-n6cccn6)nc54)ccc31)C2 ; Nc1ncccc1-c1nc2ccc(-n3cccn3)nc2n1-c1ccc2c(c1)CC[C@@H]2N1Cc2cc(O)c(C=O)cc2C1=O]

### Step 1: Synthesis of methyl 5-bromo-2-(bromomethyl)-4-methoxybenzoate

To a degassed solution of methyl 5-bromo-4-methoxy-2-methylbenzoate (2.00 g, 7.72 mmol, 1 equiv) in carbon tetrachloride (30 mL) was added N-bromosuccinimide (1.51 g, 8.49 mmol, 1 equiv) followed by AIBN (2.54 g, 15.4 mmol, 2 equiv) and acetic acid (0.93 g, 15 mmol, 2 equiv). The reaction mixture was stirred at 70° C. overnight. The reaction was quenched with water (200 mL) and extracted with ethyl acetate (200 mL). The combined organic phases were washed with brine, dried over anhydrous sodium sulfate, filtered and concentrated under reduced pressure. The residue was purified by silica gel column chromatography using a 0-40% gradient of ethyl acetate in petroleum ether to afford methyl 5-bromo-2-(bromomethyl)-4-methoxybenzoate (1.2 g, 46%) as a brown solid. MS (ESI) calcd. for C₁₀H₁₀Br₂O₃, 335.90 m/z, found 336.95 [M+H]⁺.

### Step 2: Synthesis of (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-bromo-5-methoxyisoindolin-1-one

A mixture of (S)-3-(3-(1-amino-2,3-dihydro-1H-inden-5-yl)-5-(1H-pyrazol-1-yl)-3H-imidazo[4,5-b]pyridin-2-yl)pyridin-2-amine (Intermediate 33-1) (350 mg, 0.857 mmol, 1 equiv) and methyl 5-bromo-2-(bromomethyl)-4-methoxybenzoate (579 mg, 1.71 mmol, 2 equiv) in tetrahydrofuran (4 mL) was stirred at 50° C. for 3 h. Then triethylamine (0.5 mL) was added and the resulting mixture was stirred at 50° C. for 16 h. The resulting mixture was diluted with water (100 mL) and extracted with ethyl acetate (100 mL×3). The combined extracts were concentrated under vacuum and the crude product was purified by reverse-phase flash column chromatography on C18 silica gel using a gradient of acetonitrile in water (+0.05% ammonium bicarbonate) to afford (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-bromo-5-methoxyisoindolin-1-one (290 mg, 53%) as yellow solid. MS (ESI) calcd. for C₃₂H₂₅BrN₈O₂: 632.13, found 633.13 [M+H]⁺.

### Step 3: Synthesis of (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-methoxy-3-oxoisoindoline-5-carbaldehyde

To a solution of (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-bromo-5-methoxyisoindolin-1-one (200 mg, 0.316 mmol, 1 equiv) and tert-butylisocyanide (39 mg, 0.47 mmol, 1.5 equiv) in DMSO (3 mL) were added palladium (II) acetate (7.1 mg, 0.032 mmol, 0.1 equiv) and 1,4-bis(diphenylphosphino)butane (269 mg, 0.632 mmol, 2 equiv). After stirring for 5 h at 120° C. under a nitrogen atmosphere, the mixture was allowed to cool to room temperature. The reaction was quenched with water (50 mL). The resulting mixture was extracted with ethyl acetate (3×50 mL). The combined organic layers were washed with brine (50 mL), dried over anhydrous sodium sulfate, filtered, and concentrated under reduced pressure. The residue was purified by silica gel column chromatography using a 0-10% gradient of methanol in dichloromethane to afford (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-methoxy-3-oxoisoindoline-5-carbaldehyde (100 mg, 54%) as a yellow solid. MS (ESI) calcd. for C₃₃H₂₆N₈O₃: 582.21 m/z, found 583.15 [M+H]⁺.

### Step 4: Synthesis of (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-hydroxy-3-oxoisoindoline-5-carbaldehyde (Example 208)

To a cooled (−78° C.) solution of 2-[(1S)-5-[2-(2-aminopyridin-3-yl)-5-(pyrazol-1-yl)imidazo[4,5-b]pyridin-3-yl]-2,3-dihydro-1H-inden-1-yl]-6-methoxy-3-oxo-1H-isoindole-5-carbaldehyde (100 mg, 0.172 mmol, 1 equiv) in dichloromethane (5 mL) was added boron tribromide (1M, 0.51 mL, 0.51 mmol, 3 equiv) dropwise under nitrogen atmosphere. The resulting mixture was stirred at −30° C. for 2 h. The reaction was quenched with water (10 mL) and the pH was adjusted to ˜8 with saturated aqueous sodium bicarbonate. The resulting mixture was extracted with ethyl acetate (3×50 mL). The combined organic extracts were dried over anhydrous sodium sulfate, filtered and concentrated under vacuum. The crude product was purified by preparative HPLC on a XSelect CSH OBD Column using a 17-39% gradient of acetonitrile in water (+0.05% 2,2,2-trifluoroacetic acid) to afford (S)-2-(5-(2-(2-aminopyridin-3-yl)-5-(1H-pyrazol-1-yl)-3H-imidazo[4,5-b]pyridin-3-yl)-2,3-dihydro-1H-inden-1-yl)-6-hydroxy-3-oxoisoindoline-5-carbaldehyde (Example 208) (21.5 mg, 22%) as a light yellow solid. MS (ESI) calcd. for C₃₂H₂₄N₈O₃: 568.20 m/z, found 569.30 [M+H]⁺. ¹H NMR (400 MHz, DMSO-d₆) δ (ppm): 10.29 (s, 1H), 8.36-8.48 (m, 2H), 7.96-8.13 (m, 3H), 7.73-7.87 (m, 2H), 7.51 (s, 1H), 7.31-7.39 (m, 1H), 7.22-7.30 (m, 1H), 7.17 (s, 1H), 6.81-6.91 (m, 1H), 6.51-6.60 (m, 1H), 5.83-5.94 (m, 1H), 4.44-4.58 (m, 1H), 4.03-4.16 (m, 1H), 3.05-3.18 (m, 1H), 2.90-3.03 (m, 1H), 2.31-2.42 (m, 1H), 2.12-2.28 (m, 1H).

### Example 209: N-[(1S)-5-[2-(2-aminopyridin-3-yl)-7-cyano-5-(pyrazol-1-yl)imidazo[4,5-b]pyridin-3-yl]-2,3-dihydro-1H-inden-1-yl]-3-formyl-4-hydroxybenzamide

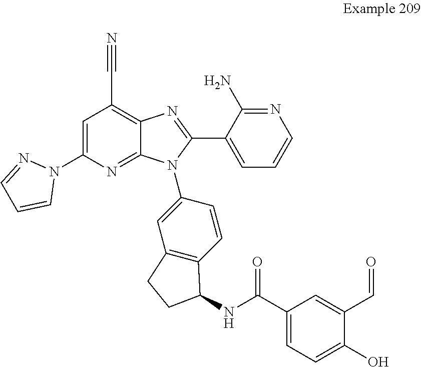 [SMILES: N#Cc1cc(-n2cccn2)nc2c1nc(-c1cccnc1N)n2-c1ccc2c(c1)CC[C@@H]2NC(=O)c1ccc(O)c(C=O)c1]

### Synthetic Route:

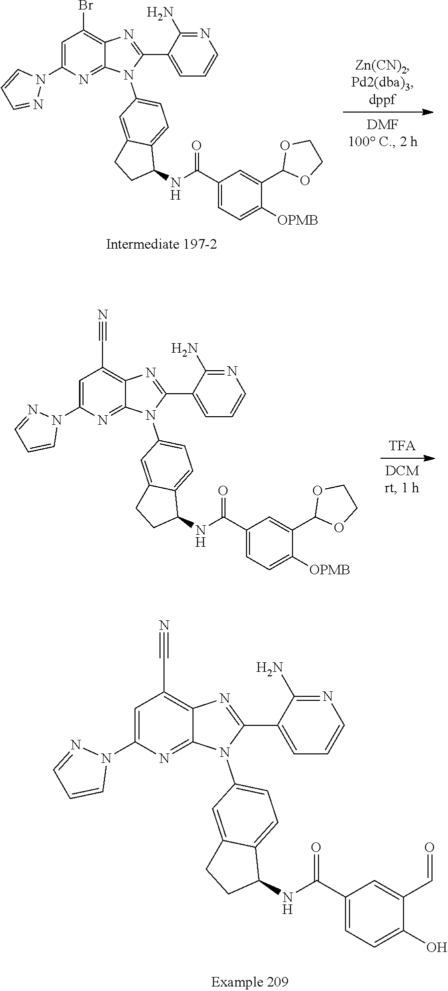

### Step 1: Synthesis of N-[(1S)-5-[2-(2-aminopyridin-3-yl)-7-cyano-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

To a solution of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (Intermediate 197-2) (100 mg, 0.125 mmol, 1 equiv) in N,N-dimethylformamide (4 mL) was added Zn(CN)₂ (22 mg, 0.19 mmol, 1.5 equiv), tris(dibenzylideneacetone)dipalladium(0) (5.7 mg, 0.006 mmol, 0.05 equiv) and dppf (6.9 mg, 0.013 mmol, 0.1 equiv). The resulting mixture was stirred under nitrogen atmosphere at 100° C. for 2 h. The reaction mixture was then treated with water (15 mL) dropwise over 10 min then extracted with ethyl acetate (15 mL×3). The combined extracts were washed with brine (10 mL), dried over anhydrous sodium sulfate, filtered, and concentrated to dryness in vacuum. The crude solid was then purified by silica gel column chromatography using a 0-10% gradient of methanol in dichloromethane to give N-[(1S)-5-[2-(2-aminopyridin-3-yl)-7-cyano-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (80 mg, 82%) as a yellow solid. MS (ESI) calcd. for C₄₂H₃₅N₉O₅: 745.28 m/z, found 746.30 [M+H]⁺.

### Step 2: Synthesis of N-[(1S)-5-[2-(2-aminopyridin-3-yl)-7-cyano-5-(pyrazol-1-yl)imidazo[4,5-b]pyridin-3-yl]-2,3-dihydro-1H-inden-1-yl]-3-formyl-4-hydroxybenzamide (Example 209)

To a solution of N-[(1S)-5-[2-(2-aminopyridin-3-yl)-7-cyano-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (80 mg, 0.11 mmol) in dichloromethane (4.5 mL) was added 2,2,2-trifluoroacetic acid (0.3 mL) and the resulting mixture was stirred at room temperature for 1 h then concentrated to dryness under reduced pressure. The crude product was then purified by preparative HPLC on a XSelect CSH Prep C18 OBD Column using a 22-44% gradient of acetonitrile in water (+0.05% 2,2,2-trifluoroacetic acid) to afford N-[(1S)-5-[2-(2-aminopyridin-3-yl)-7-cyano-5-(pyrazol-1-yl)imidazo[4,5-b]pyridin-3-yl]-2,3-dihydro-1H-inden-1-yl]-3-formyl-4-hydroxybenzamide (Example 209) (39 mg, 62%) as a yellow solid. MS (ESI) calcd. for C₃₂H₂₃N₉O₃: 581.19 m/z, found 582.20 [M+H]⁺. ¹H NMR (400 MHz, DMSO-d₆) δ (ppm): 10.28 (s, 1H), 8.30-8.35 (m, 2H), 8.27-8.29 (m, 1H), 8.09-8.15 (m, 2H), 7.88-7.89 (m, 1H), 7.80-7.82 (m, 1H), 7.40-7.43 (m, 1H), 7.35-7.37 (m, 2H), 7.06-7.08 (m, 1H), 6.81-6.82 (m, 1H), 6.61-6.79 (m, 1H), 5.57-5.61 (m, 1H), 3.02-3.03 (m, 1H), 2.89-2.91 (m, 1H), 2.50-2.51 (m, 1H), 2.06-2.11 (m, 1H).

### Example 210: N-[(1S)-5-[2-(2-aminopyridin-3-yl)-7-(methoxymethyl)-5-(pyrazol-1-yl)imidazo[4,5-b]pyridin-3-yl]-2,3-dihydro-1H-inden-1-yl]-3-formyl-4-hydroxybenzamide

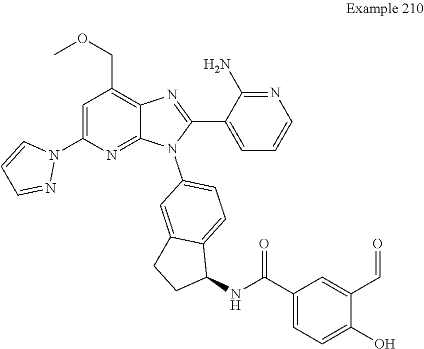 [SMILES: COCc1cc(-n2cccn2)nc2c1nc(-c1cccnc1N)n2-c1ccc2c(c1)CC[C@@H]2NC(=O)c1ccc(O)c(C=O)c1]

### Synthetic Route:

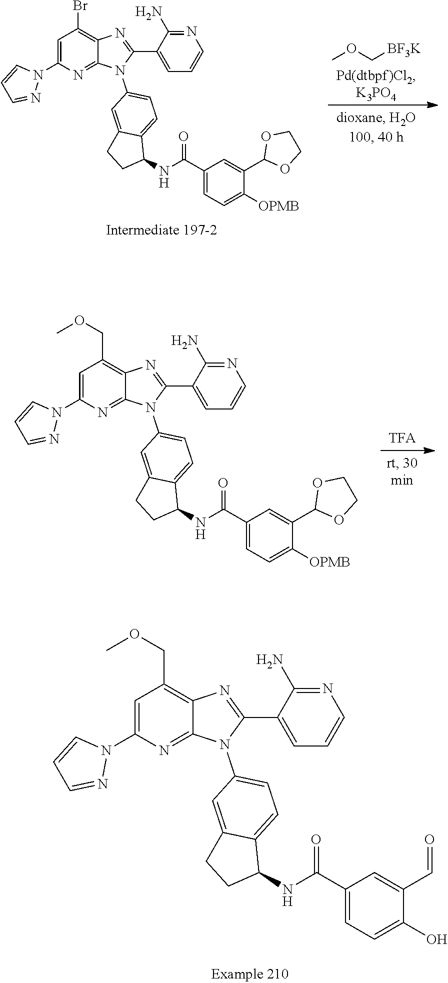

### Step 1: Synthesis of N-[(1S)-5-[2-(2-aminopyridin-3-yl)-7-(methoxymethyl)-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

A mixture of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (Intermediate 197-2) (120 mg, 0.150 mmol, 1 equiv), potassium trifluoro(methoxymethyl)boronate (68.4 mg, 0.450 mmol, 3 equiv), bis(di-tert-butylphosphino)ferrocene]dichloropalladium(II) (9.8 mg, 0.015 mmol, 0.1 equiv) and tribasic potassium phosphate (79.6 mg, 0.375 mmol, 2.5 equiv) in 1,4-dioxane (2.4 mL) and water (0.6 mL) was stirred at 100° C. for 40 h under nitrogen atmosphere. Water (50 mL) was added and the mixture was extracted with ethyl acetate (3×50 mL). The combined organic layers were dried over anhydrous sodium sulfate, filtered and concentrated under reduced pressure. The resulting crude material was purified by reverse-phase flash column chromatography on C18 silica gel using a 5-55% gradient of acetonitrile in water to afford N-[(1S)-5-[2-(2-aminopyridin-3-yl)-7-(methoxymethyl)-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (45 mg, 39%) as an orange solid. MS (ESI) calcd. for C₄₃H₄₀N₈O₆: 764.31 m/z, found 765.30 [M+H]⁺.

### Step 2: Synthesis of (S)—N-(5-(2-(2-aminopyridin-3-yl)-7-(methoxymethyl)-5-(1H-pyrazol-1-yl)-3H-imidazo[4,5-b]pyridin-3-yl)-2,3-dihydro-1H-inden-1-yl)-3-formyl-4-hydroxybenzamide (Example 210)

A solution of N-[(1S)-5-[2-(2-aminopyridin-3-yl)-7-(methoxymethyl)-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (45 mg, 0.059 mmol) in TFA (2 mL) was stirred at room temperature for 30 min. The mixture was concentrated under reduced pressure and the crude material was purified by preparative HPLC on a XSelect CSH Prep C18 OBD Column using a 19-41% gradient of acetonitrile in water (+0.1% formic acid) to afford N-[(1S)-5-[2-(2-aminopyridin-3-yl)-7-(methoxymethyl)-5-(pyrazol-1-yl)imidazo[4,5-b]pyridin-3-yl]-2,3-dihydro-1H-inden-1-yl]-3-formyl-4-hydroxybenzamide (Example 210) (5.1 mg, 15%) as an off-white solid. MS (ESI) calcd. for C₃₃H₂₈N₈O₄: 600.22 m/z, found: 601.20 [M+H]⁺. ¹H NMR (300 MHz, DMSO-d₆) δ (ppm): 10.24 (s, 1H), 8.34-8.35 (m, 1H), 8.23-8.24 (m, 1H), 7.94-8.05 (m, 2H), 7.93-7.94 (m, 1H), 7.79-7.80 (m, 1H), 7.30-7.35 (m, 2H), 7.26-7.29 (m, 2H), 6.95-6.97 (m, 1H), 6.53-6.54 (m, 1H), 6.43-6.47 (m, 1H), 5.54-5.59 (m, 1H), 4.98-5.00 (m, 2H), 3.49-3.50 (m, 3H), 2.98-3.00 (m, 1H), 2.67-2.68 (m, 1H), 2.51-2.53 (m, 1H), 2.01-2.13 (m, 1H).

### Example 211: N-[(1S)-5-[2-(2-aminopyridin-3-yl)-7-methyl-5-(1,3-oxazol-2-yl)imidazo[4,5-b]pyridin-3-yl]-2,3-dihydro-1H-inden-1-yl]-3-formyl-4-hydroxybenzamide

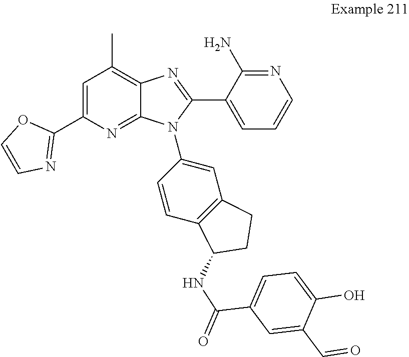 [SMILES: Cc1cc(-c2ncco2)nc2c1nc(-c1cccnc1N)n2-c1ccc2c(c1)CC[C@@H]2NC(=O)c1ccc(O)c(C=O)c1]

Example 211 was prepared in a manner analogous to Example 12 using Intermediate 211-1 in place of Intermediate 1-3 and Intermediate 57-2 in place of Intermediate 12-1. MS (ESI) calcd. for C₃₂H₂₅N₇O₄: 571.20 m/z, found 594.30 [M+Na]⁺. ¹H NMR (400 MHz, DMSO-d₆) δ (ppm): 10.30 (s, 1H), 8.93-8.95 (m, 1H), 8.30-8.31 (m, 2H), 8.23-8.24 (m, 1H), 8.06-8.09 (m, 1H), 8.00-8.02 (m, 2H), 7.37-7.41 (m, 3H), 7.30-7.35 (m, 2H), 7.96-8.02 (m, 3H), 6.43-6.46 (m, 1H), 5.64-5.66 (m, 1H), 2.99-3.01 (m, 1H), 2.95-2.99 (m, 1H), 2.74 (s, 3H), 2.50-2.51 (m, 1H), 2.05-2.11 (m, 1H).

### Intermediate 211-1: (S)-3-(3-(1-amino-2,3-dihydro-1H-inden-5-yl)-7-methyl-5-(oxazol-2-yl)-3H-imidazo[4,5-b]pyridin-2-yl)pyridin-2-amine

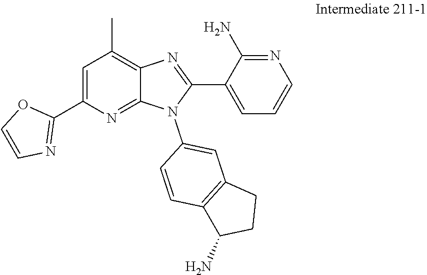 [SMILES: Cc1cc(-c2ncco2)nc2c1nc(-c1cccnc1N)n2-c1ccc2c(c1)CC[C@@H]2N]

Intermediate 211-1 was prepared in a manner analogous to Intermediate 57-1 using Intermediate 131-1 in place of Intermediate 79-1 and 2-(tributylstannyl)-1,3-oxazole in place of 2-(tributylstannyl)-1,3-thiazole. MS (ESI) calcd. for C₂₄H₂₁N₇O: 423.18 m/z, found 424.20 [M+H]⁺.

### Example 212: N-[(1S)-5-[2-(2-aminopyridin-3-yl)-7-chloro-5-(pyrazol-1-yl)imidazo[4,5-b]pyridin-3-yl]-2,3-dihydro-1H-inden-1-yl]-2,3-difluoro-5-formyl-4-hydroxybenzamide

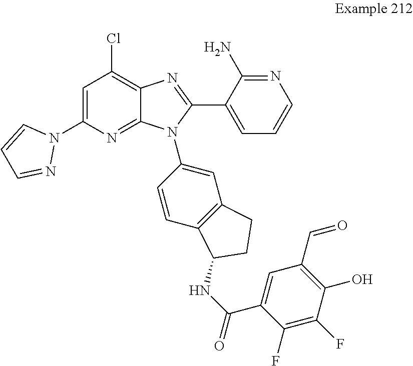 [SMILES: Nc1ncccc1-c1nc2c(Cl)cc(-n3cccn3)nc2n1-c1ccc2c(c1)CC[C@@H]2NC(=O)c1cc(C=O)c(O)c(F)c1F]

Example 212 was prepared in a manner analogous to Example 12 using Intermediate 196-1 in place of Intermediate 1-3 and Intermediate 118-1 in place of Intermediate 12-1. MS (ESI) calcd. for C₃₁H₂₁ClF₂N₈O₃: 626.14 m/z, found 627.20 [M+H]⁺. ¹H NMR (400 MHz, DMSO-d₆) δ (ppm): 10.10-10.11 (m, 1H), 8.33-8.34 (m, 1H), 7.98-8.01 (m, 2H), 7.82-7.83 (m, 1H), 7.66-7.68 (m, 1H), 7.30-7.35 (m, 4H), 6.56-6.57 (m, 1H), 6.46-6.49 (m, 1H), 5.52-5.53 (m, 1H), 2.97-2.98 (m, 1H), 2.88-2.89 (m, 1H), 2.50-2.51 (m, 1H), 2.02-2.03 (m, 1H). ¹⁹F NMR (376 MHz, DMSO-d₆) δ (ppm): −162.12, −133.46.

### Example 213: N-[(1S)-5-[2-(2-aminopyridin-3-yl)-7-fluoro-5-(pyrazol-1-yl)imidazo[4,5-b]pyridin-3-yl]-2,3-dihydro-1H-inden-1-yl]-3-formyl-4-hydroxybenzamide

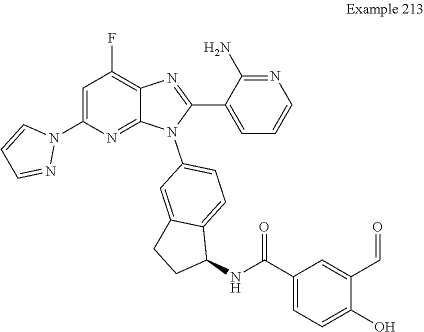 [SMILES: Nc1ncccc1-c1nc2c(F)cc(-n3cccn3)nc2n1-c1ccc2c(c1)CC[C@@H]2NC(=O)c1ccc(O)c(C=O)c1]

### Synthetic Route:

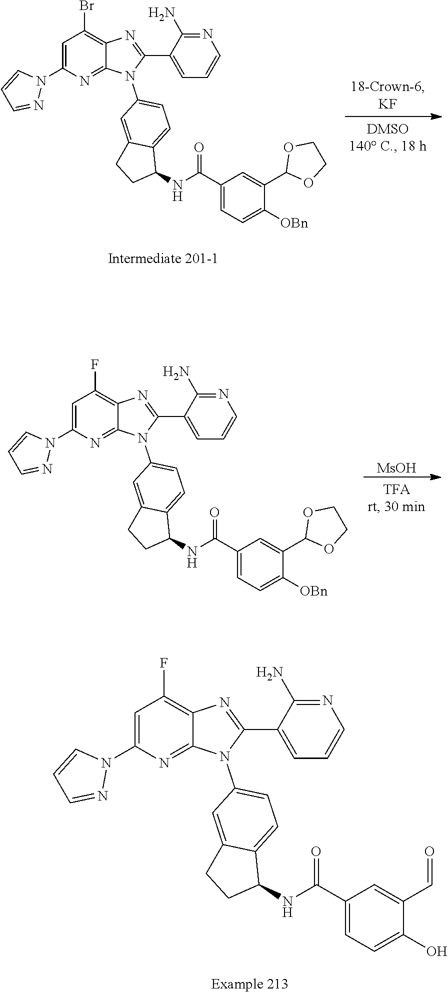

### Step 1: Synthesis of N-[(1S)-5-[2-(2-aminopyridin-3-yl)-7-fluoro-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide

To a solution of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (Intermediate 201-1) (70 mg, 0.091 mmol, 1 equiv) in DMSO (3 mL) were added 18-Crown-6 (12 mg, 0.045 mmol, 0.5 equiv) and potassium fluoride (15.9 mg, 0.273 mmol, 3 equiv). The reaction was stirred for 18 h at 140° C. under a nitrogen atmosphere. The reaction was cooled to 0° C. and quenched by the addition of water (5 mL). The aqueous layer was extracted with ethyl acetate (3×15 mL). The combined organic layers were washed with water (3×15 mL), dried over anhydrous sodium sulfate, filtered and concentrated under reduced pressure. The residue was purified by reverse-phase flash column chromatography on C18 silica gel using a 10-70% gradient of acetonitrile in wager (+0.05% ammonium bicarbonate) to afford N-[(1S)-5-[2-(2-aminopyridin-3-yl)-7-fluoro-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (40 mg, 62%) as a light yellow solid. MS (ESI) calcd. for C₄₀H₃₃FN₈O₄: 708.26 m/z, found 709.30 [M+H]⁺.

### Step 2: Synthesis of N-[(1S)-5-[2-(2-aminopyridin-3-yl)-7-fluoro-5-(pyrazol-1-yl)imidazo[4,5-b]pyridin-3-yl]-2,3-dihydro-1H-inden-1-yl]-3-formyl-4-hydroxybenzamide (Example 213)

To a cooled (0° C.) solution of N-[(1S)-5-[2-(2-aminopyridin-3-yl)-7-fluoro-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (35 mg, 0.049 mmol) in 2,2,2-trifluoroacetic acid (1 mL) was added dropwise methanesulfonic acid (0.2 mL). The reaction mixture was stirred at room temperature for 30 min. The mixture was concentrated under reduced pressure and the residue was purified by preparative HPLC on a XSelect CSH Prep C18 OBD Column using a 47-69% gradient of acetonitrile in water (+0.1% ammonium bicarbonate) to afford N-[(1S)-5-[2-(2-aminopyridin-3-yl)-7-fluoro-5-(pyrazol-1-yl)imidazo[4,5-b]pyridin-3-yl]-2,3-dihydro-1H-inden-1-yl]-3-formyl-4-hydroxybenzamide (Example 213) (4.5 mg, 15%) as a light yellow solid. MS (ESI) calcd. for C₃₁H₂₃FN₈O₃: 574.19 m/z, found 575.25 [M+H]⁺. ¹H NMR (400 MHz, DMSO-d₆) δ (ppm): 10.18 (s, 1H), 8.33-8.37 (m, 1H), 8.15-8.23 (m, 1H), 7.90-8.06 (m, 2H), 7.81-7.84 (m, 1H), 7.66-7.75 (m, 1H), 7.22-7.39 (m, 4H), 6.81-6.95 (m, 1H), 6.49-6.52 (m, 2H), 5.56-5.59 (m, 1H), 2.97-3.11 (m, 1H), 2.84-2.91 (m, 1H), 2.49-2.51 (m, 1H), 1.94-2.12 (m, 1H). ¹⁹F NMR (376 MHz, DMSO-d₆) δ (ppm): −113.70.

### Example 214: N-[(1S)-5-[2-(2-aminopyridin-3-yl)-5-(pyrazol-1-yl)imidazo[4,5-b]pyridin-3-yl]-2,3-dihydro-1H-inden-1-yl]-2-fluoro-5-formyl-4-hydroxy-3-methoxybenzamide

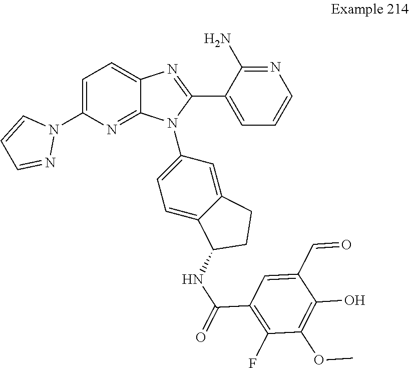 [SMILES: COc1c(O)c(C=O)cc(C(=O)N[C@H]2CCc3cc(-n4c(-c5cccnc5N)nc5ccc(-n6cccn6)nc54)ccc32)c1F]

Example 214 was prepared in a manner analogous to Example 12 using Intermediate 33-1 in place of Intermediate 1-3 and Intermediate 214-2 in place of Intermediate 12-1. MS (ESI) calcd. for C₃₂H₂₅FN₈O₄: 604.20 m/z, found 605.35 [M+H]⁺. ¹HNMR (400 MHz, DMSO-d₆) δ (ppm): 10.36 (s, 1H), 8.33-8.35 (m, 2H), 7.98-8.00 (m, 1H), 7.92-7.94 (m, 1H), 7.79 (s, 1H), 7.68-7.70 (m, 1H), 7.35-7.38 (m, 2H), 7.25-7.29 (m, 2H), 6.53-6.54 (m, 1H), 6.44-6.47 (m, 1H), 5.53-5.56 (m, 1H), 3.79 (s, 3H), 2.92-3.01 (m, 1H), 2.81-2.89 (m, 1H), 2.48-2.52 (m, 1H), 1.95-2.06 (m, 1H). ¹⁹F NMR (376 MHz, DMSO-d₆) δ (ppm): −123.17. (2,2,2-trifluoroacetic acid salt).

### Intermediate 214-2: 4-(benzyloxy)-5-(1,3-dioxolan-2-yl)-2-fluoro-3-methoxybenzoic acid

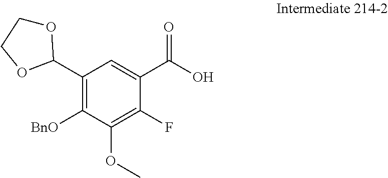 [SMILES: COc1c(F)c(C(=O)O)cc(C2OCCO2)c1OCc1ccccc1]

Intermediate 214-2 was prepared in a manner analogous to Intermediate 36-1 using Intermediate 214-1 in place of 5-bromo-2-hydroxy-3-methoxybenzaldehyde. MS (ESI) calcd. for C₁₈H₁₇FO₆: 348.10 m/z, found 349.05 [M+H]⁺.

### Intermediate 214-1: 5-bromo-4-fluoro-2-hydroxy-3-methoxybenzaldehyde

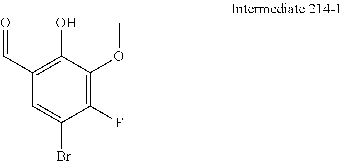

### Synthetic Route:

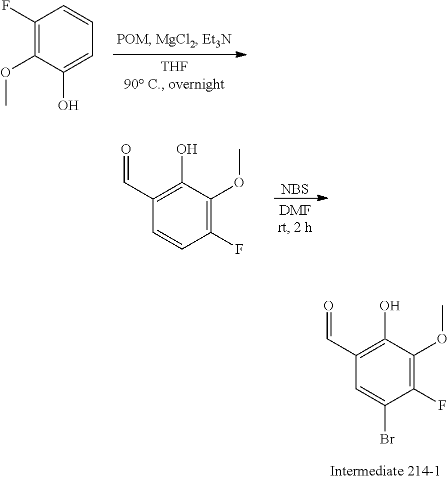

### Step 1: Synthesis of 4-fluoro-2-hydroxy-3-methoxybenzaldehyde

To a solution of 3-fluoro-2-methoxyphenol (2.00 g, 14.1 mmol, 1 equiv) in tetrahydrofuran (30 mL) was added paraformaldehyde (6.34 g, 70.4 mmol, 5 equiv), magnesium (II) chloride (2.01 g, 21.1 mmol, 1.5 equiv) and triethylamine (3.56 g, 35.2 mmol, 2.5 equiv). The resulting mixture was stirred at 90° C. for 12 h. The mixture was acidified to pH 1 with 2 M hydrochloric acid. The mixture was diluted with water (100 mL) and extracted with ethyl acetate (300 mL×3). The combined extracts were washed with brine (30 mL), dried over anhydrous sodium sulfate, filtered, and concentrated to dryness in vacuo. The resulting oil was then subjected to silica gel column chromatography using a 0-25% gradient of ethyl acetate in petroleum ether to afford 4-fluoro-2-hydroxy-3-methoxybenzaldehyde (750 mg, 31%) as a yellow oil. MS (ESI) calcd. for C₈H₇FO₃: 170.04 m/z, found 169.00 [M−H]⁻.

### Step 2: Synthesis of 5-bromo-4-fluoro-2-hydroxy-3-methoxybenzaldehyde (Intermediate 214-1)

To a cooled (0° C.) solution of 4-fluoro-2-hydroxy-3-methoxybenzaldehyde (700 mg, 4.11 mmol, 1 equiv) in N,N-dimethylformamide (15 mL) was added N-bromosuccinimide (805 mg, 4.53 mmol, 1.1 equiv). The resulting mixture was stirred at room temperature for 2 h. The mixture was diluted with water (100 mL) and extracted with ethyl acetate (100 mL×3). The combined extracts were washed with brine (30 mL), dried over anhydrous sodium sulfate, filtered, and concentrated to dryness in vacuo. The resulting crude was purified by reverse phase flash column chromatography on C18 silica gel using a 10-70% gradient of acetonitrile in water (+0.05% ammonium bicarbonate) to afford 5-bromo-4-fluoro-2-hydroxy-3-methoxybenzaldehyde (Intermediate 214-1) (190 mg, 19%) as a yellow oil. MS (ESI) calcd. for C₈H₆BrFO₃: 247.95 m/z, found 248.75 [M+H]⁺.

### Example 215: N-[(1S)-5-[2-(2-aminopyridin-3-yl)-6-methoxy-5-(pyrazol-1-yl)imidazo[4,5-b]pyridin-3-yl]-2,3-dihydro-1H-inden-1-yl]-3-formyl-4-hydroxybenzamide

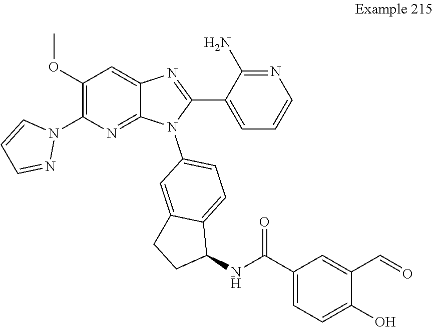 [SMILES: COc1cc2nc(-c3cccnc3N)n(-c3ccc4c(c3)CC[C@@H]4NC(=O)c3ccc(O)c(C=O)c3)c2nc1-n1cccn1]

Example 215 was prepared in a manner analogous to Example 7 using Intermediate 215-2 in place of Intermediate 1-3 and Intermediate 57-2 in place of Intermediate 5-1. MS (ESI) calcd. for C₃₂H₂₆N₈O₄: 586.21 m/z, found 587.20 [M+H]⁺. ¹H NMR (400 MHz, DMSO-d₆) δ (ppm): 10.00-10.35 (m, 1H), 8.20-8.35 (m, 1H), 7.95-8.12 (m, 4H), 7.55-7.72 (m, 1H), 7.25-7.40 (m, 3H), 7.15-7.25 (m, 1H), 6.95-7.10 (m, 1H), 6.35-6.65 (m, 2H), 5.40-5.80 (m, 1H), 3.85-3.95 (m, 3H), 2.95-3.10 (m, 1H), 2.75-2.95 (m, 1H), 2.35-2.50 (m, 1H), 1.85-2.05 (m, 1H).

### Intermediate 215-2: (S)-3-(3-(1-amino-2,3-dihydro-1H-inden-5-yl)-6-methoxy-5-(1H-pyrazol-1-yl)-3H-imidazo[4,5-b]pyridin-2-yl)pyridin-2-amine

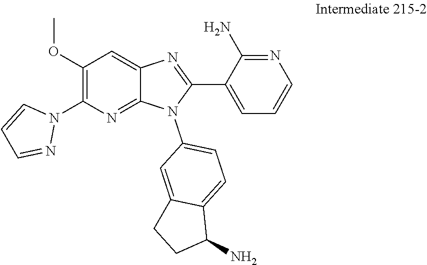 [SMILES: COc1cc2nc(-c3cccnc3N)n(-c3ccc4c(c3)CC[C@@H]4N)c2nc1-n1cccn1]

Intermediate 215-2 was prepared in a manner analogous to Intermediate 13-2 (starting from Step 2) using Intermediate 215-1 in place of the nitro starting material. MS (ESI) calcd. for C₂₄H₂₂N₈O: 438.19 m/z, found 439.20 [M+H]⁺.

### Intermediate 215-1: (S)—N-(5-((5-methoxy-3-nitro-6-(1H-pyrazol-1-yl)pyridin-2-yl)amino)-2,3-dihydro-1H-inden-1-yl)acetamide

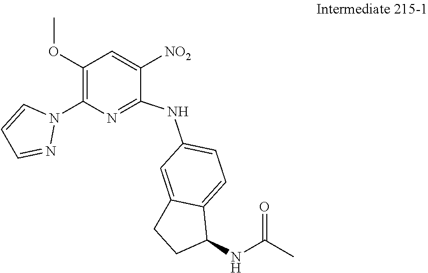 [SMILES: COc1cc([N+](=O)[O-])c(Nc2ccc3c(c2)CC[C@@H]3NC(C)=O)nc1-n1cccn1]

### Synthetic Route:

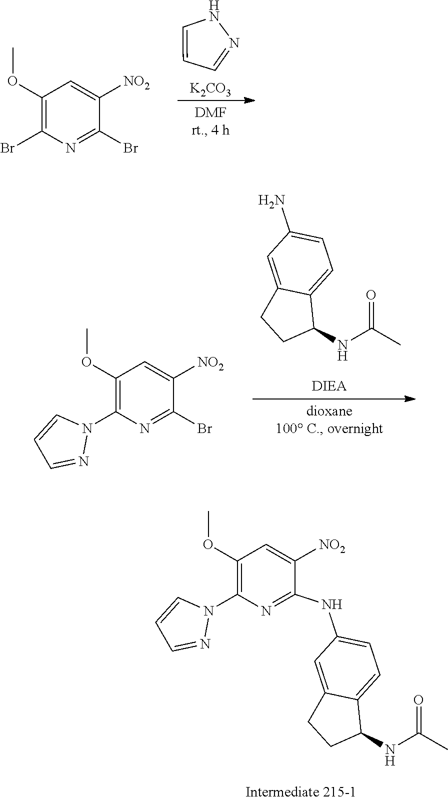

### Step 1: 2-bromo-5-methoxy-3-nitro-6-(pyrazol-1-yl)pyridine

To a stirred solution of 2,6-dibromo-3-methoxy-5-nitropyridine (5.00 g, 16.0 mmol, 1 equiv) and pyrazole (4.37 g, 64.1 mmol, 4 equiv) in N,N-dimethylformamide (40 mL) was added K₂CO₃ (4.43 g, 32.1 mmol, 2 equiv) and the resulting mixture was stirred for 4 h at room temperature. The reaction was then quenched by the addition of 300 mL of water. The precipitated solids were collected by filtration and washed with water (3×100 mL). The solid collected was purified by silica gel column chromatography using a 30-40% gradient of ethyl acetate in petroleum ether to afford 2-bromo-5-methoxy-3-nitro-6-(pyrazol-1-yl)pyridine (1.9 g, 38%) as a green solid. MS (ESI) calcd. for C₉H₇BrN₄O₃: 297.97 m/z, found 298.00 [M+H]⁺.

### Step 2: N-[(1S)-5-{[5-methoxy-3-nitro-6-(pyrazol-1-yl)pyridin-2-yl]amino}-2,3-dihydro-1H-inden-1-yl]acetamide (Intermediate 215-1)

To a stirred solution of 2-bromo-5-methoxy-3-nitro-6-(pyrazol-1-yl)pyridine (1.9 g, 6.4 mmol, 1 equiv) and N-[(1S)-5-amino-2,3-dihydro-1H-inden-1-yl]acetamide HCl (1.21 g, 6.4 mmol, 1 equiv) in 1,4-dioxane (15 mL) was added N,N-diisopropylethylamine (4.11 g, 31.8, 5 equiv mmol) and the resulting mixture was stirred overnight at 100° C. The reaction mixture was cooled to room temperature and diluted with ethyl acetate/petroleum ether (1:1). The precipitated solids were collected by filtration and washed with petroleum ether/ethyl acetate (1:1) (3×200 mL). The resulting solid was dried by air to afford N-[(1S)-5-{[5-methoxy-3-nitro-6-(pyrazol-1-yl)pyridin-2-yl]amino}-2,3-dihydro-1H-inden-1-yl]acetamide (Intermediate 215-1) (1.5 g, 36%). MS (ESI) calcd. for C₂₀H₂₀N₆O₄: 408.15 m/z, found 409.10 [M+H]⁺.

### Example 216: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-(2-fluoro-4-formyl-3-hydroxyphenyl)acetamide; trifluoroacetic acid

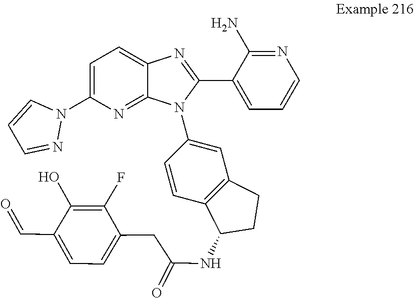 [SMILES: Nc1ncccc1-c1nc2ccc(-n3cccn3)nc2n1-c1ccc2c(c1)CC[C@@H]2NC(=O)Cc1ccc(C=O)c(O)c1F]

Example 216 was prepared in a manner analogous to Example 12 using Intermediate 33-1 in place of Intermediate 1-3 and Intermediate 216-1 in place of Intermediate 12-1. MS (ESI) calcd. for C₃₂H₂₅FN₈O₃: 588.20 m/z, found 589.15 [M+H]⁺. ¹H NMR (300 MHz, DMSO-d₆) δ (ppm): 10.21 (s, 1H), 8.41-8.47 (m, 1H), 8.33-8.40 (m, 1H), 8.04-8.11 (m, 1H), 7.97-8.04 (m, 1H), 7.80-7.86 (m, 1H), 7.70-7.79 (m, 1H), 7.42-7.51 (m, 2H), 7.30-7.36 (m, 2H), 6.92-7.02 (m, 1H), 6.74-6.84 (m, 1H), 6.54-6.61 (m, 1H), 5.30-5.40 (m, 1H), 3.65 (s, 2H), 2.92-3.05 (m, 1H), 2.81-2.91 (m, 1H), 2.47-2.51 (m, 1H), 1.82-2.01 (m, 1H). ¹⁹F NMR (282 MHz, DMSO-d₆) δ (ppm): −139.26. (2,2,2-trifluoroacetic acid salt).

### Intermediate 216-1: 2-(2-fluoro-4-formyl-3-hydroxyphenyl)acetic acid

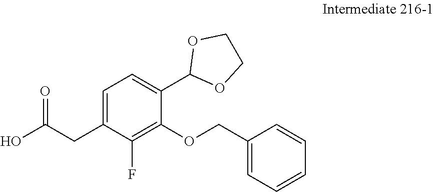 [SMILES: O=C(O)Cc1ccc(C2OCCO2)c(OCc2ccccc2)c1F]

Intermediate 216-1 was prepared in a manner analogous to Intermediate 6-2 (via Intermediate 6-1) starting from 4-bromo-3-fluoro-2-hydroxybenzaldehyde in place of 4-bromo-2-hydroxybenzaldehyde and using the benzyl protecting group instead of p-methoxybenzyl (see Step 1 of Intermediate 12-1 for a representative benzyl protection procedure). MS (ESI) calcd. for C₁₈H₁₇FO₅: 332.11 m/z, found 331.10 [M−H]⁻.

### Example 217: N-[(1S)-5-[2-(2-aminopyridin-3-yl)-5-(pyrazol-1-yl)imidazo[4,5-b]pyridin-3-yl]-2,3-dihydro-1H-inden-1-yl]-2-fluoro-5-formyl-4-methanesulfonamidobenzamide

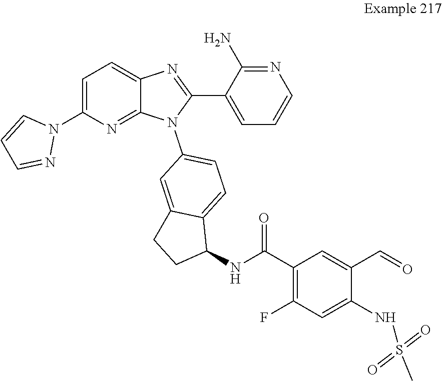 [SMILES: CS(=O)(=O)Nc1cc(F)c(C(=O)N[C@H]2CCc3cc(-n4c(-c5cccnc5N)nc5ccc(-n6cccn6)nc54)ccc32)cc1C=O]

Example 217 was prepared in a manner analogous to Example 12 using Intermediate 33-1 in place of Intermediate 1-3, Intermediate 217-1 in place of Intermediate 12-1 and dichloromethane/2,2,2-trifluoroacetic acid (3:1) in place of 2,2,2-trifluoroacetic acid/methanesulfonic acid. MS (ESI) calcd. for C₃₂H₂₆FN₉O₄S: 651.18 m/z, found 652.25 [M+H]⁺. ¹H NMR (400 MHz, DMSO-d₆) δ (ppm): 9.93 (s, 1H), 8.39-8.44 (m, 2H), 7.99-8.04 (m, 3H), 7.84-7.86 (m, 1H), 7.78-7.80 (m, 1H), 7.41-7.45 (m, 2H), 7.28-7.33 (m, 2H), 7.13-7.25 (m, 1H), 6.82-6.85 (m, 1H), 6.60-6.62 (m, 1H), 5.25-5.29 (m, 1H), 4.36 (s, 3H), 2.96-2.98 (m, 1H), 2.85-2.90 (m, 1H), 2.45-2.47 (m, 1H), 1.84-1.85 (m, 1H). ¹⁹F NMR (376 MHz, DMSO-d₆) δ (ppm): −98.86. (2,2,2-trifluoroacetic acid salt).

### Intermediate 217-1: 5-(1,3-dioxolan-2-yl)-2-fluoro-4-(methylsulfonamido)benzoic acid

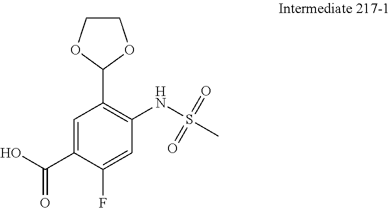 [SMILES: CS(=O)(=O)Nc1cc(F)c(C(=O)O)cc1C1OCCO1]

### Synthetic Route:

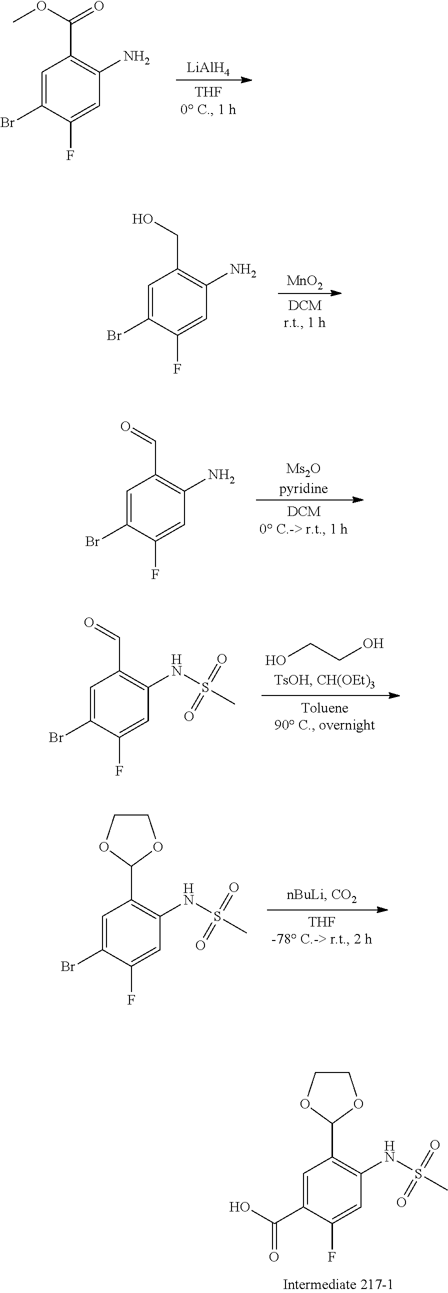

### Step 1: Synthesis of (2-amino-5-bromo-4-fluorophenyl) methanol

To a cooled (0° C.) solution of methyl 2-amino-5-bromo-4-fluorobenzoate (3.00 g, 12.1 mmol, 1 equiv) in tetrahydrofuran (30 mL) was added LiAlH₄ (1.15 g, 30.2 mmol, 2.5 equiv) and the resulting solution was stirred at 0° C. for 1 h. The reaction was quenched with water (50 mL) at 0° C. and the mixture was extracted with ethyl acetate (3×150 mL). The combined organic extracts were washed with brine (3×100 mL), dried over anhydrous Na₂SO₄, filtered and concentrated under vacuum to yield (2-amino-5-bromo-4-fluorophenyl)methanol (2.5 g, 94%) as a light yellow solid. MS (ESI) calcd. for C₇H₇BrFNO: 218.97 m/z, found 219.15 [M+H]⁺.

### Step 2: Synthesis of 2-amino-5-bromo-4-fluorobenzaldehyde

To a cooled (0° C.) solution of (2-amino-5-bromo-4-fluorophenyl)methanol (2.5 g, 11 mmol, 1 equiv) in dichloromethane (30 mL) was added MnO₂ (9.88 g, 114 mmol, 10 equiv) and the resulting mixture was stirred for 1 h at room temperature. The mixture was filtered, rinsing with dichloromethane (3×50 mL). The filtrate was concentrated under reduced pressure giving 2-amino-5-bromo-4-fluorobenzaldehyde (2 g, 81%) as a yellow solid. MS (ESI) calcd. for C₇H₅BrFNO: 216.95 m/z, found 217.15 [M+H]⁺.

### Step 3: Synthesis of N-(4-bromo-5-fluoro-2-formylphenyl)methanesulfonamide

To a cooled (0° C.) solution of 2-amino-5-bromo-4-fluorobenzaldehyde (2.1 g, 9.6 mmol, 1 equiv) in pyridine (20 mL) was added methanesulfonic anhydride (8.39 g, 48.2 mmol, 4 equiv) and the resulting mixture was stirred for 1 h at room temperature. The reaction was quenched by the addition of water (20 mL) at 0° C. The aqueous layer was extracted with ethyl acetate (3×100 mL). The combined organic layers were washed with water (3×100 mL), dried over anhydrous sodium sulfate, filtered and concentrated under reduced pressure. The residue was purified by silica gel column chromatography using a 0-50% gradient of ethyl acetate in petroleum ether to afford N-(4-bromo-5-fluoro-2-formylphenyl)methanesulfonamide (2.5 g, 88%) as a light yellow solid. MS (ESI) calcd. for C₈H₇BrFNO₃S: 294.93 m/z, found 295.15 [M+H]⁺.

### Step 4: Synthesis of N-[4-bromo-2-(1,3-dioxolan-2-yl)-5-fluorophenyl]methanesulfonamide

To a solution of N-(4-bromo-5-fluoro-2-formylphenyl)methanesulfonamide (2.1 g, 7.1 mmol, 1 equiv) in toluene (20 mL) was added ethylene glycol (2.20 g, 35.5 mmol, 5 equiv), triethyl orthoformate (3.15 g, 21.3 mmol, 3 equiv) and p-toluenesulfonic acid (0.12 g, 0.71 mmol, 0.1 equiv). The resulting mixture was stirred overnight at 90° C. The reaction was quenched by the addition of water (15 mL) at 0° C. The aqueous layer was extracted with ethyl acetate (3×50 mL). The combined organic layers were washed with water (3×50 mL), dried over anhydrous sodium sulfate, filtered and concentrated under reduced pressure. The residue was purified by silica gel column chromatography using a 0-30% gradient of ethyl acetate in petroleum ether to afford N-[4-bromo-2-(1,3-dioxolan-2-yl)-5-fluorophenyl]methanesulfonamide (1 g, 41%) as a light yellow solid. MS (ESI) calcd. for C₁₀H₁₁BrFNO₄S: 338.96 m/z, found 339.15[M+H]⁺.

### Step 5: Synthesis of 5-(1,3-dioxolan-2-yl)-2-fluoro-4-methanesulfonamidobenzoic acid (Intermediate 217-1)

To a cooled (−78° C.) solution of N-[4-bromo-2-(1,3-dioxolan-2-yl)-5-fluorophenyl]methanesulfonamide (1.00 g, 2.94 mmol, 1 equiv) in tetrahydrofuran (20 mL) was added nBuLi (2.94 mL, 2.5 M in hexane, 2.5 equiv) and the resulting mixture was stirred for 1 h under a nitrogen atmosphere. The atmosphere was exchanged with CO₂ and stirring was continued at −78° C. for another 1 h. The reaction was quenched by the addition of saturated aqueous ammonium chloride (20 mL) at 0° C. The resulting mixture was concentrated under reduced pressure and the residue was purified by reverse-phase flash column chromatography on C18 silica gel using a 10-60% gradient of acetonitrile in water (+0.05% ammonium bicarbonate) to afford 5-(1,3-dioxolan-2-yl)-2-fluoro-4-methanesulfonamidobenzoic acid (Intermediate 217-1) (0.7 g, 78%) as a light yellow solid. MS (ESI) calcd. for C₁₁H₁₂FNO₆S: 305.04 m/z, found 306.10 [M+H]⁺.

### Example 218: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-azido-3-fluoro-5-formyl-4-hydroxybenzamide

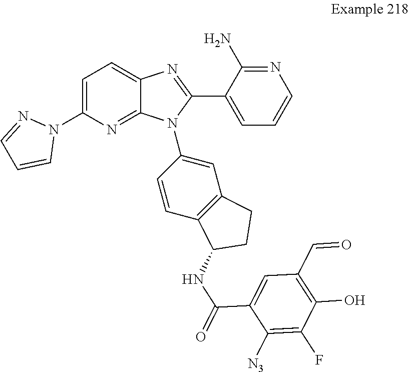 [SMILES: [N-]=[N+]=Nc1c(C(=O)N[C@H]2CCc3cc(-n4c(-c5cccnc5N)nc5ccc(-n6cccn6)nc54)ccc32)cc(C=O)c(O)c1F]

### Synthetic Route:

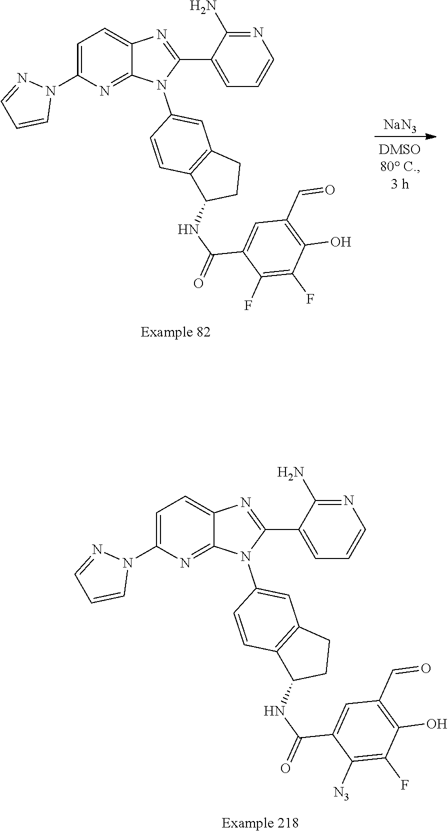 [SMILES: Nc1ncccc1-c1nc2ccc(-n3cccn3)nc2n1-c1ccc2c(c1)CC[C@@H]2NC(=O)c1cc(C=O)c(O)c(F)c1F ; [N-]=[N+]=Nc1c(C(=O)N[C@H]2CCc3cc(-n4c(-c5cccnc5N)nc5ccc(-n6cccn6)nc54)ccc32)cc(C=O)c(O)c1F ; "NaN3"]

### Step 1: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-azido-3-fluoro-5-formyl-4-hydroxybenzamide (Example 218)

To a solution of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2,3-difluoro-5-formyl-4-hydroxybenzamide (Example 82) (500 mg, 0.844 mmol, 1 equiv) in DMSO (5 mL) was added sodium azide (66 mg, 1.0 mmol, 1.2 equiv) and the resulting mixture was stirred at 80° C. for 3 h. The mixture was diluted with water and 2,2,2-trifluoroacetic acid and was purified by preparative HPLC on a Phenomenex Gemini C18 column using a 10-80% gradient of acetonitrile in water (+0.05% 2,2,2-trifluoroacetic acid) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-azido-3-fluoro-5-formyl-4-hydroxybenzamide (Example 218) (400 mg, 80%) as a yellow solid. MS (ESI) calculated for C₃₁H₂₂FN₁₁O₃: 615.19 m/z, found 616.30 [M+H]⁺. ¹H NMR (400 MHz, DMSO-d₆) δ (ppm): 10.22 (s, 1H), 8.88 (d, J=8.2 Hz, 1H), 8.43 (d, J=8.6 Hz, 1H), 8.39-8.36 (m, 1H), 8.09 (dd, J=5.9, 1.7 Hz, 1H), 8.01 (d, J=8.6 Hz, 1H), 7.83 (dd, J=1.6, 0.7 Hz, 1H), 7.77-7.71 (m, 2H), 7.48-7.44 (m, 2H), 7.39 (dd, J=8.1, 1.9 Hz, 1H), 6.80 (dd, J=7.6, 5.9 Hz, 1H), 6.58-6.52 (m, 1H), 5.56 (dd, J=8.2, 8.2 Hz, 1H), 3.04 (ddd, J=16.2, 8.8, 2.9 Hz, 1H), 2.92 (ddd, J=16.1, 8.4, 8.4 Hz, 1H), 2.63-2.51 (m, 1H), 2.06 (dddd, J=12.5, 8.9, 8.9, 8.9 Hz, 1H). (2,2,2-trifluoroacetic acid salt).

### Example 219: N-(5-(2-(2-aminopyridin-3-yl)-5-(1H-pyrazol-1-yl)-3H-imidazo[4,5-b]pyridin-3-yl)-1-methyl-2,3-dihydro-1H-inden-1-yl)-3-formyl-4-hydroxybenzamide

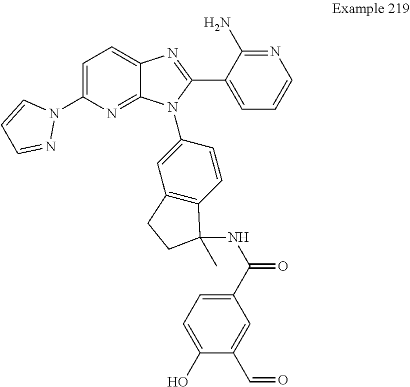 [SMILES: CC1(NC(=O)c2ccc(O)c(C=O)c2)CCc2cc(-n3c(-c4cccnc4N)nc4ccc(-n5cccn5)nc43)ccc21]

### Synthetic Route:

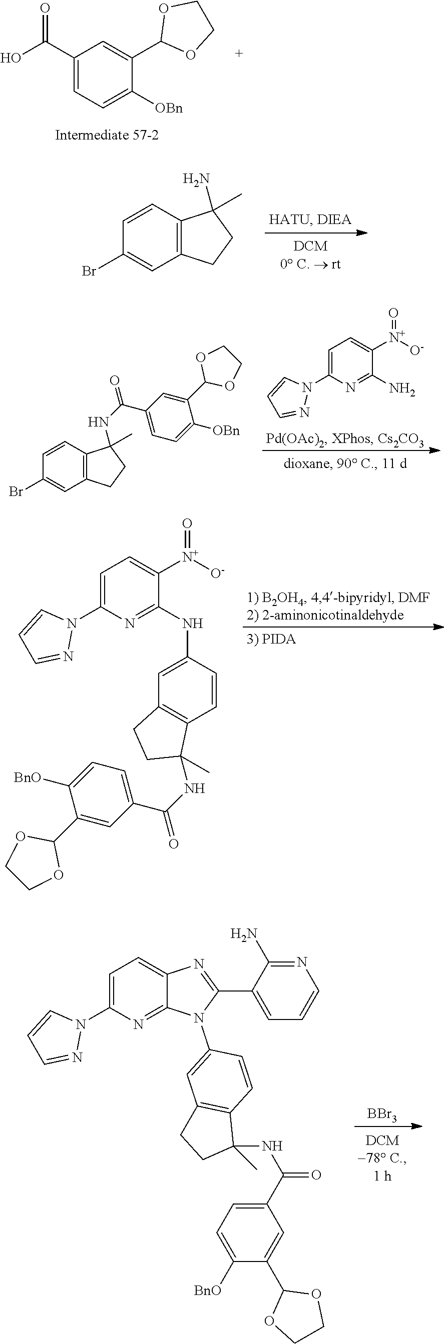

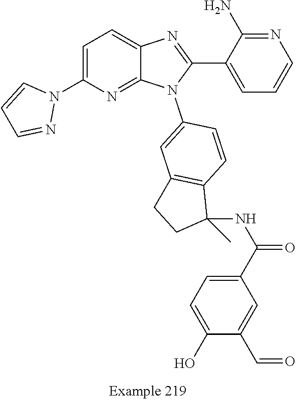 [SMILES: CC1(NC(=O)c2ccc(O)c(C=O)c2)CCc2cc(-n3c(-c4cccnc4N)nc4ccc(-n5cccn5)nc43)ccc21]

### Step 1: Synthesis of 4-(benzyloxy)-N-(5-bromo-1-methyl-2,3-dihydro-1H-inden-1-yl)-3-(1,3-dioxolan-2-yl)benzamide

To a cooled (0° C.) solution of 4-(benzyloxy)-3-(1,3-dioxolan-2-yl)benzoic acid (Intermediate 57-2) (858 mg, 2.86 mmol, 1.5 equiv) in dichloromethane (10 mL) was added HATU (1.01 g, 2.67 mmol, 1.4 equiv) and N,N-diisopropylethylamine (1.33 mL, 7.62 mmol, 4 equiv) and the resulting mixture was stirred at 0° C. for 1 h. 5-bromo-1-methyl-2,3-dihydro-1H-inden-1-amine-HCl (500 mg, 1.9 mmol, 1 equiv) was added and the mixture was stirred overnight at room temperature. The mixture was diluted with ethyl acetate and washed with saturated aqueous sodium bicarbonate, water (×2) and brine. The organic phase was dried over magnesium sulfate, filtered and concentrated in vacuo. The crude material was purified by silica gel column chromatography using a 0-50% gradient of ethyl acetate in hexanes to afford 4-(benzyloxy)-N-(5-bromo-1-methyl-2,3-dihydro-1H-inden-1-yl)-3-(1,3-dioxolan-2-yl)benzamide (767 mg, 79%) as a white solid. MS (ESI) calculated for C₂₇H₂₆BrNO₄: 507.10, found 508.15 [M+H]⁺.

### Step 2: Synthesis of 4-(benzyloxy)-3-(1,3-dioxolan-2-yl)-N-(1-methyl-5-((3-nitro-6-(1H-pyrazol-1-yl)pyridin-2-yl)amino)-2,3-dihydro-1H-inden-1-yl)benzamide

A mixture of 4-(benzyloxy)-N-(5-bromo-1-methyl-2,3-dihydro-1H-inden-1-yl)-3-(1,3-dioxolan-2-yl)benzamide (767 mg, 1.51 mmol, 1 equiv), 3-nitro-6-(1-pyrazolyl)-2-pyridinamine (371 mg, 1.81 mmol, 1.2 equiv), cesium carbonate (688 mg, 2.11 mmol, 1.4 equiv), XPhos (108 mg, 0.226 mmol, 0.15 equiv), Pd(OAc)₂ (17 mg, 0.075 mmol, 0.05 equiv) and 1,4-dioxane (12 mL) was sparged with nitrogen for 5 min then stirred at 90° C. under nitrogen for 1 week. Additional Pd(OAc)₂ (100 mg, 0.446 mmol, 0.3 equiv) was added, the mixture was sparged with nitrogen for 5 min and stirring was continued for 4 days at 90° C. The mixture was diluted with ethyl acetate, washed with brine, dried over magnesium sulfate, filtered and concentrated under reduced pressure. The crude material was purified by silica gel column chromatography using a 0-80% gradient of ethyl acetate in hexanes to afford 4-(benzyloxy)-3-(1,3-dioxolan-2-yl)-N-(1-methyl-5-((3-nitro-6-(1H-pyrazol-1-yl)pyridin-2-yl)amino)-2,3-dihydro-1H-inden-1-yl)benzamide (406 mg, 43%) as an orange/brown solid. MS (ESI) calculated for C₃₅H₃₂N₆O₆: 632.24 m/z, found 633.25 [M+H]⁺.

### Step 3: Synthesis of N-(5-(2-(2-aminopyridin-3-yl)-5-(1H-pyrazol-1-yl)-3H-imidazo[4,5-b]pyridin-3-yl)-1-methyl-2,3-dihydro-1H-inden-1-yl)-4-(benzyloxy)-3-(1,3-dioxolan-2-yl)benzamide

To a cooled (0° C.) solution of 4-(benzyloxy)-3-(1,3-dioxolan-2-yl)-N-(1-methyl-5-((3-nitro-6-(1H-pyrazol-1-yl)pyridin-2-yl)amino)-2,3-dihydro-1H-inden-1-yl)benzamide (304 mg, 0.48 mmol, 1 equiv) and 4,4′-bipyridyl (3.8 mg, 24 umol, 0.05 equiv) in N,N-dimethylformamide (3 mL) was added B₂(OH)₄ (129 mg, 1.44 mmol, 3 equiv) and the resulting mixture was stirred for 30 min at room temperature. 2-Aminonicotinaldehyde (76 mg, 0.63 mmol, 1.3 equiv) was added and the resulting mixture was stirred for 30 min at room temperature. The mixture was diluted with 1,4-dioxane (0.75 mL) and filtered. Acetoxyphenyliodo acetate (464 mg, 1.44 mmol, 3 equiv) was added and the mixture was stirred for 1 h at room temperature. The mixture was concentrated in vacuo and taken up into ethyl acetate. The mixture was washed with water and brine, dried over magnesium sulfate, filtered and concentrated in vacuo. The crude material was purified by silica gel column chromatography using a 0-100% gradient of ethyl acetate in hexanes to afford N-(5-(2-(2-aminopyridin-3-yl)-5-(1H-pyrazol-1-yl)-3H-imidazo[4,5-b]pyridin-3-yl)-1-methyl-2,3-dihydro-1H-inden-1-yl)-4-(benzyloxy)-3-(1,3-dioxolan-2-yl)benzamide (79 mg, 23%) as a brown solid. MS (ESI) calculated for C₄₁H₃₆N₈O₄: 704.29 m/z, found 705.40 [M+H]⁺.

### Step 4: Synthesis of N-(5-(2-(2-aminopyridin-3-yl)-5-(1H-pyrazol-1-yl)-3H-imidazo[4,5-b]pyridin-3-yl)-1-methyl-2,3-dihydro-1H-inden-1-yl)-3-formyl-4-hydroxybenzamide (Example 219)

To a cooled (−78° C.) solution of N-(5-(2-(2-aminopyridin-3-yl)-5-(1H-pyrazol-1-yl)-3H-imidazo[4,5-b]pyridin-3-yl)-1-methyl-2,3-dihydro-1H-inden-1-yl)-4-(benzyloxy)-3-(1,3-dioxolan-2-yl)benzamide (70 mg, 99 umol, 1 equiv) in dichloromethane (1 mL) was added boron tribromide (1M in heptane, 1 mL, 10 equiv) dropwise and the resulting mixture was stirred at −78° C. for 1 h. The reaction was quenched with saturated aqueous sodium bicarbonate at −78° C. and the mixture was allowed to warm to room temperature and diluted with ethyl acetate. The layers were separated and the organic phase was washed with brine, concentrated in vacuo and taken up into DMSO. The solution was filtered and purified by preparative HPLC on a Phenomenex Gemini C18 column using a 50-100% gradient of acetonitrile in water (+0.5% ammonium bicarbonate) to afford N-(5-(2-(2-aminopyridin-3-yl)-5-(1H-pyrazol-1-yl)-3H-imidazo[4,5-b]pyridin-3-yl)-1-methyl-2,3-dihydro-1H-inden-1-yl)-3-formyl-4-hydroxybenzamide (Example 219) (7 mg, 9%) as an off white solid. MS (ESI) calculated for C₃₂H₂₆N₈O₃: 570.21, found 571.30 [M+H]⁺. ¹H NMR (400 MHz, DMSO-d₆) δ 10.28 (s, 1H), 9.88 (s, 1H), 9.03 (s, 1H), 8.75 (dd, J=4.1, 1.8 Hz, 1H), 8.44 (d, J=2.6 Hz, 1H), 8.34 (dd, J=8.4, 1.8 Hz, 1H), 8.25-8.22 (m, 2H), 8.16 (d, J=2.4 Hz, 1H), 7.95 (dd, J=8.7, 2.4 Hz, 1H), 7.87 (d, J=1.6 Hz, 1H), 7.51-7.43 (m, 3H), 7.39 (d, J=8.0 Hz, 1H), 7.24 (dd, J=8.4, 4.2 Hz, 1H), 6.99 (d, J=8.7 Hz, 1H), 6.64 (dd, J=2.6, 1.7 Hz, 1H), 3.05 (dt, J=14.8, 6.9 Hz, 1H), 2.87 (dt, J=15.4, 7.2 Hz, 1H), 2.72 (ddd, J=14.2, 8.6, 5.9 Hz, 1H), 2.07 (ddd, J=13.2, 8.3, 5.5 Hz, 1H), 1.64 (s, 3H).

### Example 220: (S)-3-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-7-hydroxy-4-oxo-3,4-dihydroquinazoline-6-carbaldehyde

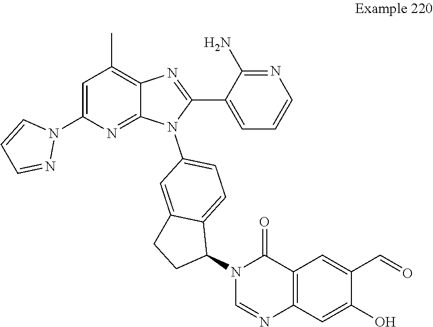 [SMILES: Cc1cc(-n2cccn2)nc2c1nc(-c1cccnc1N)n2-c1ccc2c(c1)CC[C@@H]2n1cnc2cc(O)c(C=O)cc2c1=O]

### Synthetic Route:

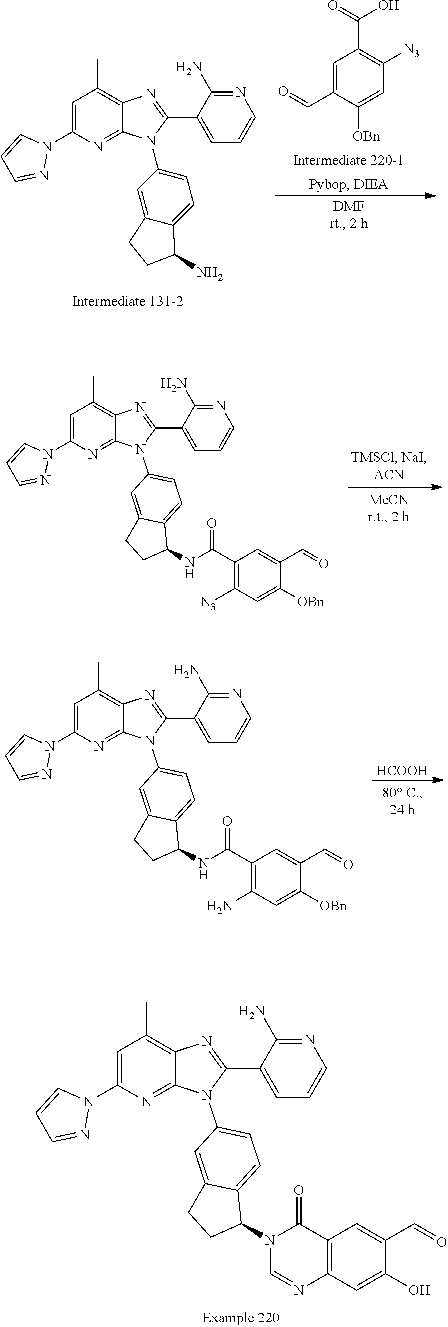

### Step 1: Synthesis of (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-2-azido-4-(benzyloxy)-5-formylbenzamide

To a solution of 2-azido-4-(benzyloxy)-5-formylbenzoic acid (Intermediate 220-1) (300 mg, 1.01 mmol, 1 equiv) and 3-{3-[(1S)-1-amino-2,3-dihydro-1H-inden-5-yl]-7-methyl-5-(pyrazol-1-yl)imidazo[4,5-b]pyridin-2-yl}pyridin-2-amine (Intermediate 131-2) (426 mg, 1.01 mmol, 1 equiv) in N,N-dimethylformamide (5 mL) were added PyBOP (525 mg, 1.01 mmol, 1 equiv) and N,N-diisopropylethylamine (261 mg, 2.02 mmol, 2 equiv) and the resulting solution was stirred at room temperature for 2 h. The reaction was quenched with water (50 mL) and the precipitated solids were collected by filtration and dried under air to obtain (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-2-azido-4-(benzyloxy)-5-formylbenzamide (150 mg, 21%) as an orange solid. MS (ESI) calcd. for C₃₉H₃₁N₁₁O₃: 701.26 m/z, found 702.35 [M+H]⁺.

### Step 2: Synthesis of (S)-2-amino-N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-4-(benzyloxy)-5-formylbenzamide

To a solution of (S)—N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-2-azido-4-(benzyloxy)-5-formylbenzamide (150 mg, 0.214 mmol, 1 equiv) in acetonitrile (15 mL) was added NaI (64 mg, 0.43 mmol, 2 equiv) and the resulting mixture was stirred at room temperature for 30 min. TMSCl (46 mg, 0.43 mmol, 2 equiv) was added and stirring was continued for 2 h. The solids formed were collected by filtration and recrystallized from acetonitrile to give (S)-2-amino-N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-4-(benzyloxy)-5-formylbenzamide (120 mg, 83%) as a yellow solid. MS (ESI) calcd. for C₃₉H₃₃N₉O₃: 675.27 m/z, found 676.15 [M+H]⁺.

### Step 3: Synthesis of (S)-3-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-7-hydroxy-4-oxo-3,4-dihydroquinazoline-6-carbaldehyde (Example 220)

(S)-2-amino-N-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-4-(benzyloxy)-5-formylbenzamide (140 mg, 0.207 mmol) was dissolved in formic acid (10 mL) and the resulting solution was stirred at 80° C. for 24 h. The reaction mixture was concentrated to dryness and the resulting residue was dissolved in N,N-dimethylformamide and purified by preparative HPLC on a XSelect CSH Fluoro Phenyl Column using a 20-36% gradient of acetonitrile in water (+0.1% formic acid) to obtain (S)-3-(5-(2-(2-aminopyridin-3-yl)-7-methyl-5-(1H-pyrazol-1-yl)-3H-imidazo[4,5-b]pyridin-3-yl)-2,3-dihydro-1H-inden-1-yl)-7-hydroxy-4-oxo-3,4-dihydroquinazoline-6-carbaldehyde (Example 220) (2.7 mg, 2%) as an off-white solid. MS (ESI) calcd. for C₃₃H₂₅N₉O₃: 595.21 m/z, found 618.25 [M+Na]⁺. ¹H NMR (400 MHz, DMSO-d₆) δ (ppm): 10.32 (s, 1H), 8.30-8.60 (m, 2H), 7.90-8.10 (m, 2H), 7.65-7.90 (m, 2H), 7.40-7.60 (m, 1H), 7.30-7.40 (m, 1H), 7.15-7.30 (m, 2H), 6.90-7.30 (m, 1H), 6.50-6.70 (m, 1H), 6.40-6.50 (m, 1H), 6.15-6.40 (m, 1H), 3.15-3.33 (m, 1H), 2.90-3.15 (m, 1H), 2.66-2.90 (m, 4H), 2.15-2.40 (m, 1H).

### Intermediate 220-1: 2-azido-4-(benzyloxy)-5-formylbenzoic acid

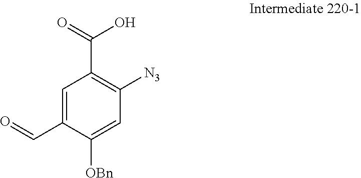 [SMILES: [N-]=[N+]=Nc1cc(OCc2ccccc2)c(C=O)cc1C(=O)O]

### Synthetic Route:

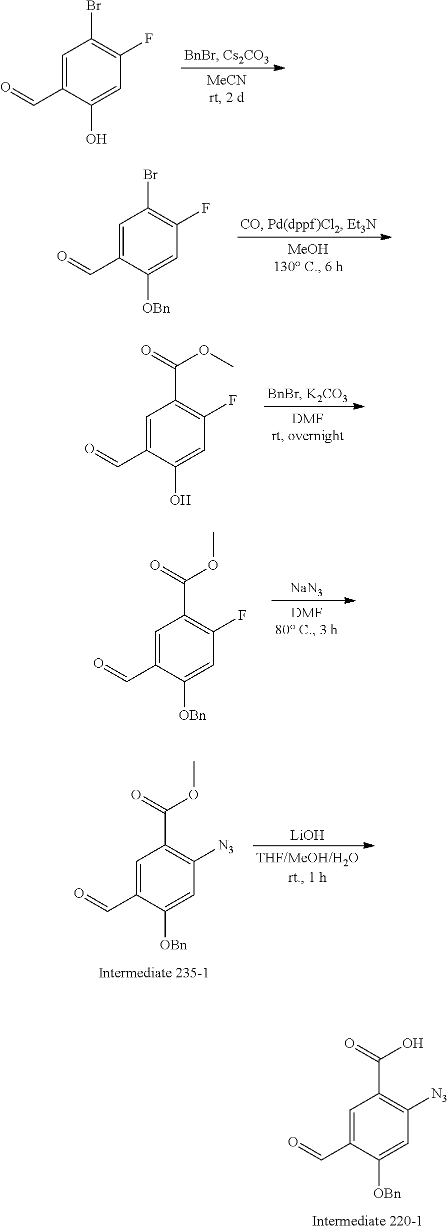

### Step 1: 2-(benzyloxy)-5-bromo-4-fluorobenzaldehyde

To a mixture of 5-bromo-4-fluoro-2-hydroxybenzaldehyde (300 g, 1.37 mol) and Cs₂CO₃ (1.34 kg, 4.10 mol, 3 equiv) in acetonitrile (3 L) was added benzyl bromide (351.43 g, 2.055 mol, 1.5 equiv) and the resulting mixture was stirred overnight at room temperature. The resulting mixture was filtered, rinsing with ethyl acetate (500 mL). The filtrate was concentrated under reduced pressure. The crude product was re-crystallized from petroleum ether (500 mL) to afford 2-(benzyloxy)-5-bromo-4-fluorobenzaldehyde (320 g, 76%) as a white solid. ¹H-NMR (400 MHz, DMSO-d₆) δ (ppm): 10.24 (s, 1H), 7.80-8.00 (m, 1H), 7.48-7.60 (m, 3H), 7.25-7.48 (m, 3H), 5.15-5.45 (m, 2H).

### Step 2: methyl 2-fluoro-5-formyl-4-hydroxybenzoate

To a solution of 2-(benzyloxy)-5-bromo-4-fluorobenzaldehyde (320 g, 1.04 mol, 1 equiv) in methanol (7.5 L) was added [1,1′-bis(diphenylphosphino)ferrocene]dichloropalladium(II) (30.0 g, 36.8 mmol, 0.35 equiv) and triethylamine (314 g, 3.11 mol, 3 equiv) and the reaction mixture was stirred for 6 h at 130° C. under 20 atm of carbon monoxide. The resulting mixture was allowed to cool to room temperature, depressurized and concentrated under vacuum. The residue was acidified to pH 3 with HCl (2M). The resulting mixture was extracted with ethyl acetate (2 L×3). The combined organic layers were dried over anhydrous sodium sulfate, filtered and concentrated under reduced pressure. The residue obtained was purified by silica gel column chromatography using a 0-20% gradient of ethyl acetate/dichloromethane (1:1) in petroleum ether. The crude product was re-crystallized from petroleum ether (300 mL) to afford methyl 2-fluoro-5-formyl-4-hydroxybenzoate (118 g, 57%) as an off-white solid. MS (ESI) calcd. for C₉H₇FO₄: 198.03 m/z, found 197.05 [M−H]⁻.

### Step 3: methyl 4-(benzyloxy)-2-fluoro-5-formylbenzoate

To a mixture of methyl 2-fluoro-5-formyl-4-hydroxybenzoate (118 g, 596 mmol, 1 equiv) and K₂CO₃ (165.8 g, 1.191 mol, 2 equiv) in N,N-dimethylformamide (500 mL) was added (bromomethyl)benzene (152.78 g, 893.27 mmol, 1.5 equiv) and the resulting mixture was stirred overnight at room temperature. The resulting mixture was diluted with ethyl acetate (2 L), washed with water (1.2 L×4), dried over anhydrous sodium sulfate, filtered and concentrated under reduced pressure. The crude product was re-crystallized from petroleum ether (500 mL) and further purified by silica gel column chromatography using a 0-20% gradient of ethyl acetate/dichloromethane (1:1) in petroleum ether to obtain methyl 4-(benzyloxy)-2-fluoro-5-formylbenzoate (111 g, 65%) as a white solid. MS (ESI) calcd. for C₁₆H₁₃N₃O₄: 311.09 m/z, found 312.10 [M+H]⁺.

### Step 4: Synthesis of methyl 2-azido-4-(benzyloxy)-5-formylbenzoate

To a solution of methyl 4-(benzyloxy)-2-fluoro-5-formylbenzoate (1.00 g, 3.47 mmol, 1 equiv) in N,N-dimethylformamide (10 mL) was added NaN₃ (0.34 g, 5.2 mmol. 1.5 equiv) and the resulting mixture was stirred at 80° C. for 3 h. The reaction was quenched with water (100 mL) and the precipitated solids were collected by filtration and dried by air to obtain methyl 2-azido-4-(benzyloxy)-5-formylbenzoate (800 mg, 74%) as an off-white solid. MS (ESI) calcd. for C₁₆H₁₃N₃O₄: 311.09 m/z, found 312.00 [M+H]⁺.

### Step 5: Synthesis of 2-azido-4-(benzyloxy)-5-formylbenzoic acid (Intermediate 220-1)

To a solution of methyl 2-azido-4-(benzyloxy)-5-formylbenzoate (800 mg, 2.57 mmol, 1 equiv) in THF/MeOH (5:1, 12 mL) was added 2N LiOH (aqueous) (5.14 mL, 10.3 mmol, 4 equiv) and the resulting solution was stirred at room temperature for 1 h. The mixture was concentrated under reduced pressure. The mixture was adjusted to pH-6 with 2N HCl (aqueous) and the precipitated solids were collected by filtration and used directly in subsequent transformations without any further purification (650 mg, 79%). MS (ESI) calcd. for C₁₅H₁₁N₃O₄: 297.07 m/z, found 295.85 [M−H]⁻.

### Example 221: N-[(1S)-5-[2-(2-aminopyridin-3-yl)-7-methoxy-5-(pyrazol-1-yl)imidazo[4,5-b]pyridin-3-yl]-2,3-dihydro-1H-inden-1-yl]-2-fluoro-5-formyl-4-hydroxybenzamide

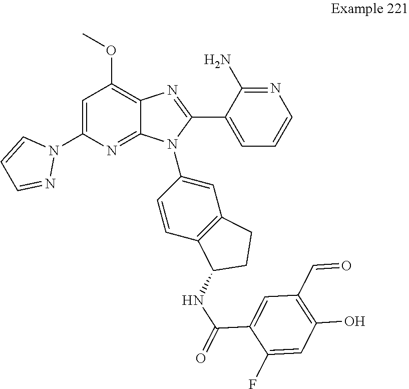 [SMILES: COc1cc(-n2cccn2)nc2c1nc(-c1cccnc1N)n2-c1ccc2c(c1)CC[C@@H]2NC(=O)c1cc(C=O)c(O)cc1F]

Example 221 was prepared in a manner analogous to Example 12 using Intermediate 200-3 in place of Intermediate 1-3 and Intermediate 16-1 in place of Intermediate 12-1. MS (ESI) calcd. for C₃₂H₂₅FN₈O₄: 604.20 m/z, found 627.30 [M+Na]⁺. ¹H NMR (300 MHz, DMSO-d₆) δ (ppm): 10.21 (s, 1H), 8.34-8.35 (m, 1H), 8.00-8.04 (m, 2H), 7.81-7.82 (m, 1H), 7.66-7.68 (m, 1H), 7.53-7.54 (m, 1H), 7.38-7.41 (m, 2H), 7.31-7.33 (m, 1H), 6.83-6.86 (m, 1H), 6.73-6.77 (m, 1H), 6.54-6.55 (m, 1H), 5.52-5.56 (m, 1H), 4.19-4.20 (m, 3H), 2.99-3.04 (m, 1H), 2.87-2.93 (m, 1H), 2.57-2.67 (m, 1H), 2.01-2.06 (m, 1H). ¹⁹F-NMR (400 MHz, DMSO-d₆) δ (ppm): −101.81.

### Example 222: N-[(1S)-5-[2-(2-aminopyridin-3-yl)-7-methoxy-5-(pyrazol-1-yl)imidazo[4,5-b]pyridin-3-yl]-2,3-dihydro-1H-inden-1-yl]-2,3-difluoro-5-formyl-4-hydroxybenzamide

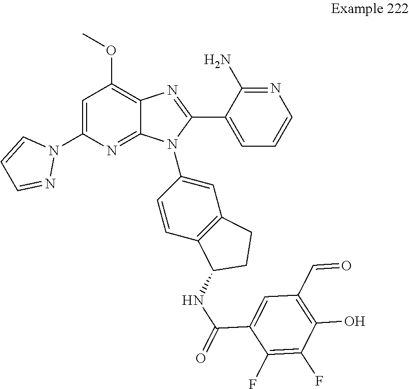 [SMILES: COc1cc(-n2cccn2)nc2c1nc(-c1cccnc1N)n2-c1ccc2c(c1)CC[C@@H]2NC(=O)c1cc(C=O)c(O)c(F)c1F]

Example 222 was prepared in a manner analogous to Example 12 using Intermediate 200-3 in place of Intermediate 1-3 and Intermediate 118-1 in place of Intermediate 12-1. MS (ESI) calcd. for C₃₂H₂₄F₂N₈O₄: 622.19 m/z, found: 623.30 [M+H]⁺. ¹H NMR (300 MHz, DMSO-d₆) δ (ppm): 10.19-10.21 (m, 1H), 8.31-8.34 (m, 1H), 8.03-8.05 (m, 1H), 7.81-7.83 (m, 2H), 7.68-7.70 (m, 1H), 7.51-7.54 (m, 1H), 7.40-7.42 (m, 2H), 7.31-7.33 (m, 1H), 6.75-6.78 (m, 1H), 6.51-6.55 (m, 1H), 5.52-5.56 (m, 1H), 4.19-4.20 (m, 3H), 2.99-3.05 (m, 1H), 2.86-2.94 (m, 1H), 2.57-2.67 (m, 1H), 1.98-2.08 (m, 1H). ¹⁹F-NMR (400 MHz, DMSO-d₆) δ (ppm): −128.37, −158.17.

### Example 223: N-[(1S)-5-[2-(2-aminopyridin-3-yl)-7-(4-methylpiperazin-1-yl)-5-(pyrazol-1-yl)imidazo[4,5-b]pyridin-3-yl]-2,3-dihydro-1H-inden-1-yl]-3-formyl-4-hydroxybenzamide

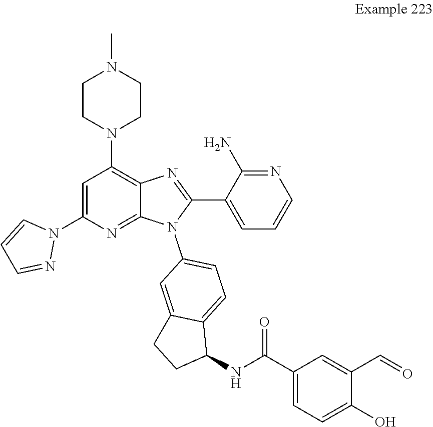 [SMILES: CN1CCN(c2cc(-n3cccn3)nc3c2nc(-c2cccnc2N)n3-c2ccc3c(c2)CC[C@@H]3NC(=O)c2ccc(O)c(C=O)c2)CC1]

### Synthetic Route:

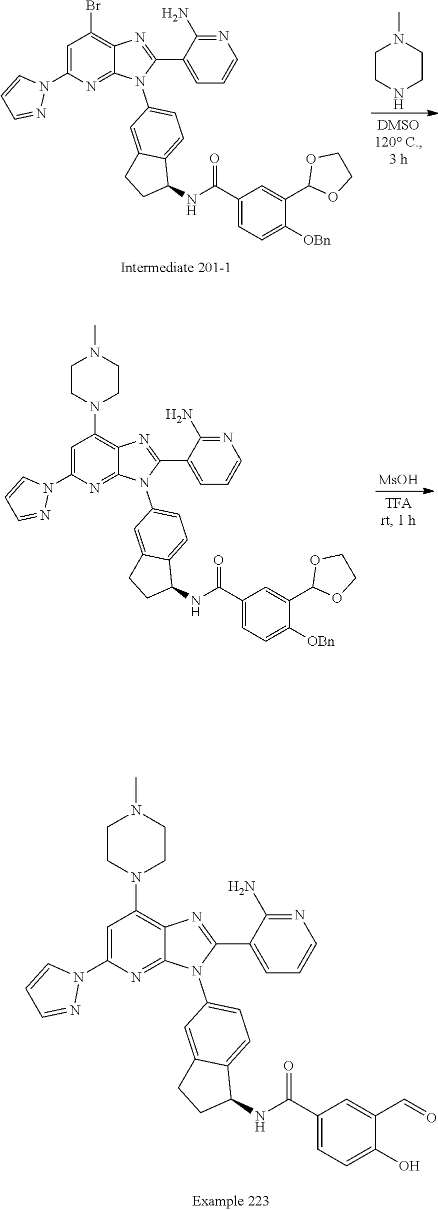

### Step 1: Synthesis of N-[(1S)-5-[2-(2-aminopyridin-3-yl)-7-(4-methylpiperazin-1-yl)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide

A solution of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (Intermediate 201-1) (190 mg, 0.247 mmol, 1 equiv) and 1-methylpiperazine (247 mg, 2.47 mmol, 10 equiv) in DMSO (3 mL) was stirred at 120° C. for 3 h. The reaction was quenched with water (50 mL). The resulting mixture was extracted with ethyl acetate (20 mL×3). The combined organic layers were washed with brine, dried over anhydrous sodium sulfate, filtered and concentrated under reduced pressure to afford N-[(1S)-5-[2-(2-aminopyridin-3-yl)-7-(4-methylpiperazin-1-yl)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (200 mg, 93%) as a red solid. MS (ESI) calcd. for C₄₅H₄₄N₁₀O₄: 788.36 m/z, found 789.45 [M+H]⁺.

### Step 2: Synthesis of N-[(1S)-5-[2-(2-aminopyridin-3-yl)-7-(4-methylpiperazin-1-yl)-5-(pyrazol-1-yl)imidazo[4,5-b]pyridin-3-yl]-2,3-dihydro-1H-inden-1-yl]-3-formyl-4-hydroxybenzamide (Example 223)

To a solution of N-[(1S)-5-[2-(2-aminopyridin-3-yl)-7-(4-methylpiperazin-1-yl)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (170 mg, 0.215 mmol) in TFA (2.5 mL) was added methanesulfonic acid (0.5 mL) and the mixture was stirred at 25° C. for 1 h. The mixture was basified to pH 7 with saturated aqueous sodium bicarbonate and the resulting mixture was extracted with ethyl acetate (50 mL×2). The combined organic layers were washed with brine, dried over anhydrous sodium sulfate, filtered and concentrated under reduced pressure. The crude product was purified by preparative HPLC on a XSelect CSH Prep C18 OBD Column using a 2-25% gradient of acetonitrile in water (+0.1% formic acid) to afford N-[(1S)-5-[2-(2-aminopyridin-3-yl)-7-(4-methylpiperazin-1-yl)-5-(pyrazol-1-yl)imidazo[4,5-b]pyridin-3-yl]-2,3-dihydro-1H-inden-1-yl]-3-formyl-4-hydroxybenzamide (Example 223) (24.4 mg, 17% yield) as a white solid. MS (ESI) calcd. for C₃₆H₃₄N₁₀O₃: 654.28 m/z, found 655.40 [M+H]⁺. ¹H NMR (400 MHz, DMSO-d₆) δ (ppm): 10.25 (s, 1H), 8.24-8.25 (m, 2H), 7.95-8.06 (m, 2H), 7.71-7.72 (m, 1H), 7.28-7.29 (m, 2H), 7.17-7.21 (m, 3H), 7.02-7.05 (m, 1H), 6.42-6.48 (m, 2H), 5.56-5.60 (m, 1H), 3.97-3.98 (m, 4H), 2.96-2.98 (m, 1H), 2.85-2.88 (m, 1H), 2.55-2.56 (m, 4H), 2.47-2.50 (m, 1H), 2.25-2.26 (m, 3H), 2.05-2.08 (m, 1H).

### Example 224: N-[(1S)-5-[2-(2-aminopyridin-3-yl)-7-(morpholin-4-yl)-5-(pyrazol-1-yl)imidazo[4,5-b]pyridin-3-yl]-2,3-dihydro-1H-inden-1-yl]-3-formyl-4-hydroxybenzamide

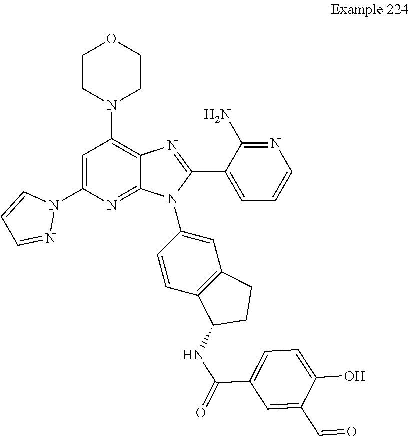 [SMILES: Nc1ncccc1-c1nc2c(N3CCOCC3)cc(-n3cccn3)nc2n1-c1ccc2c(c1)CC[C@@H]2NC(=O)c1ccc(O)c(C=O)c1]

Example 224 was prepared in a manner analogous to Example 223 using morpholine in place of 1-methylpiperazine. MS (ESI) calcd. for C₃₅H₃₁N₉O₄: 641.25 m/z, found 642.40 [M+H]⁺. ¹H NMR (400 MHz, DMSO-d₆) δ (ppm): 10.27 (s, 1H), 8.26-8.28 (m, 2H), 8.06-8.12 (m, 1H), 7.96-7.97 (m, 1H), 7.73-7.74 (m, 1H), 7.27-7.31 (m, 3H), 7.19-7.22 (m, 2H), 7.05-7.07 (m, 1H), 6.47-6.51 (m, 2H), 5.56-5.60 (m, 1H), 3.94-3.95 (m, 4H), 3.81-3.82 (m, 4H), 2.97-2.99 (m, 1H), 2.86-2.88 (m, 1H), 2.47-2.50 (m, 1H), 2.03-2.09 (m, 1H).

### Example 225: N-[(1S)-5-[2-(2-aminopyridin-3-yl)-7-(difluoromethoxy)-5-(pyrazol-1-yl)imidazo[4,5-b]pyridin-3-yl]-2,3-dihydro-1H-inden-1-yl]-3-formyl-4-hydroxybenzamide

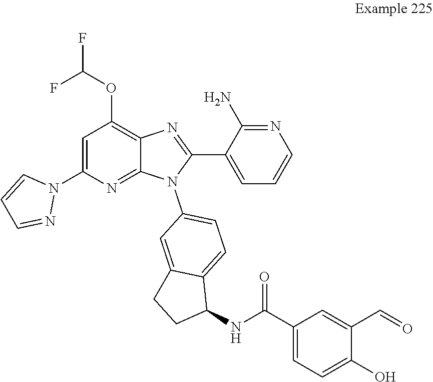 [SMILES: Nc1ncccc1-c1nc2c(OC(F)F)cc(-n3cccn3)nc2n1-c1ccc2c(c1)CC[C@@H]2NC(=O)c1ccc(O)c(C=O)c1]

### Synthetic Route:

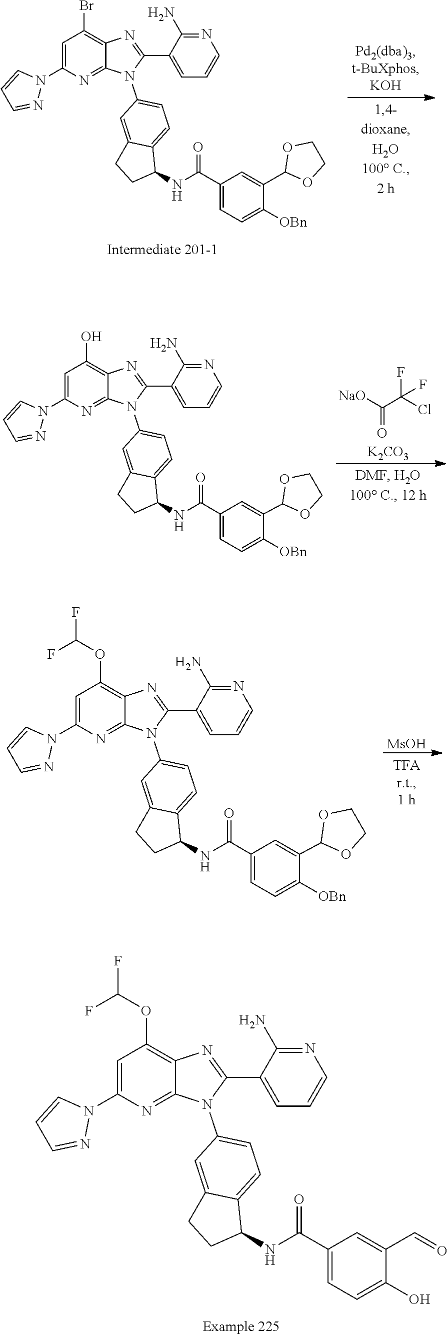

### Step 1: Synthesis of N-[(1S)-5-[2-(2-aminopyridin-3-yl)-7-hydroxy-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide

To a solution of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (Intermediate 201-1) (120 mg, 0.156 mmol, 1 equiv) in 1,4-dioxane (8 mL) and water (2 mL) were added Pd₂(dba)₃ (14 mg, 0.016 mmol, 0.1 equiv), t-BuXPhos (6.6 mg, 0.016 mmol, 0.1 equiv) and KOH (17 mg, 0.31 mmol, 2 equiv). The resulting mixture was maintained under nitrogen and stirred for 2 h at 100° C. The resulting mixture was purified by reverse phase flash chromatography on C18 silica gel using a 5-80% gradient of acetonitrile in water (+0.05% ammonium bicarbonate) to afford N-[(1S)-5-[2-(2-aminopyridin-3-yl)-7-hydroxy-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (90 mg, 79%) as a white solid. MS (ESI) calcd. for C₄₀H₃₄N₈O₅: 706.27 m/z, found 707.30 [M+H]⁺.

### Step 2: Synthesis of N-[(1S)-5-[2-(2-aminopyridin-3-yl)-7-(difluoromethoxy)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide

To a solution of N-[(1S)-5-[2-(2-aminopyridin-3-yl)-7-hydroxy-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (90 mg, 0.13 mmol, 1 equiv) in N,N-dimethylformamide (10 mL) and water (2 mL) were added sodium 2-chloro-2,2-difluoroacetate (39 mg, 0.25 mmol, 2 equiv) and K₂CO₃ (53 mg, 0.38 mmol, 3 equiv) and the resulting mixture was maintained under nitrogen and stirred for 12 h at 100° C. The resulting mixture was purified by reverse-phase flash column chromatography on C18 silica gel using a 5-90% gradient of acetonitrile in water (+0.1% formic acid) to afford N-[(1S)-5-[2-(2-aminopyridin-3-yl)-7-(difluoromethoxy)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (40 mg, 36.73% yield) as a white solid. MS (ESI) calcd. for C₄₁H₃₄F₂N₈O₅: 756.26 m/z, found 757.35 [M+H]⁺.

### Step 3: Synthesis of N-[(1S)-5-[2-(2-aminopyridin-3-yl)-7-(difluoromethoxy)-5-(pyrazol-1-yl)imidazo[4,5-b]pyridin-3-yl]-2,3-dihydro-1H-inden-1-yl]-3-formyl-4-hydroxybenzamide (Example 225)

To a solution of N-[(1S)-5-[2-(2-aminopyridin-3-yl)-7-(difluoromethoxy)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (40 mg, 0.053 mmol) in TFA (2 mL) was added methanesulfonic acid (0.4 mL). The resulting mixture was stirred at room temperature for 1 h. The resulting mixture were concentrated under vacuum to dryness to give a brown oil. The crude product was purified by preparative HPLC on a XSelect CSH Prep C18 OBD Column using a 25-48% gradient of acetonitrile in water (+0.1% formic acid) to afford N-[(1S)-5-[2-(2-aminopyridin-3-yl)-7-(difluoromethoxy)-5-(pyrazol-1-yl)imidazo[4,5-b]pyridin-3-yl]-2,3-dihydro-1H-inden-1-yl]-3-formyl-4-hydroxybenzamide (Example 225) (9.5 mg, 29%) as a white solid. MS (ESI) calcd. for C₃₂H₂₄F₂N₈O₄: 622.19 m/z, found 623.20 [M+H]⁺. ¹H NMR (400 MHz, DMSO-d₆+D₂O) δ (ppm): 10.26 (s, 1H), 8.96-8.98 (m, 1H), 8.32-8.33 (m, 2H), 8.21-8.27 (m, 1H), 8.05-8.07 (m, 1H), 7.81-7.97 (m, 1H), 7.63 (s, 1H), 7.27-7.36 (m, 4H), 7.05-7.07 (m, 1H), 6.56 (s, 1H), 6.47-6.48 (m, 1H), 5.58-5.60 (m, 1H), 3.00-3.02 (m, 1H), 2.87-2.90 (m, 1H), 2.38-2.51 (m, 1H), 2.04-2.07 (m, 1H). ¹⁹F NMR (376 MHz, DMSO-d₆+D₂O) 6-83.44.

### Example 226: N-[(1S)-5-[2-(2-aminopyridin-3-yl)-6-hydroxy-5-(pyrazol-1-yl)imidazo[4,5-b]pyridin-3-yl]-2,3-dihydro-1H-inden-1-yl]-2-fluoro-5-formyl-4-hydroxybenzamide

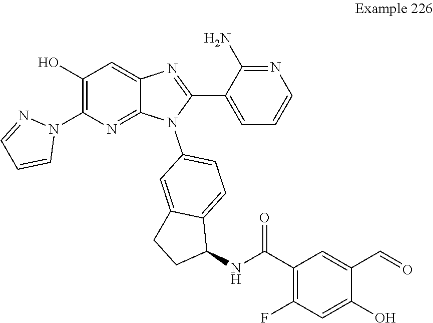 [SMILES: Nc1ncccc1-c1nc2cc(O)c(-n3cccn3)nc2n1-c1ccc2c(c1)CC[C@@H]2NC(=O)c1cc(C=O)c(O)cc1F]

### Synthetic Route:

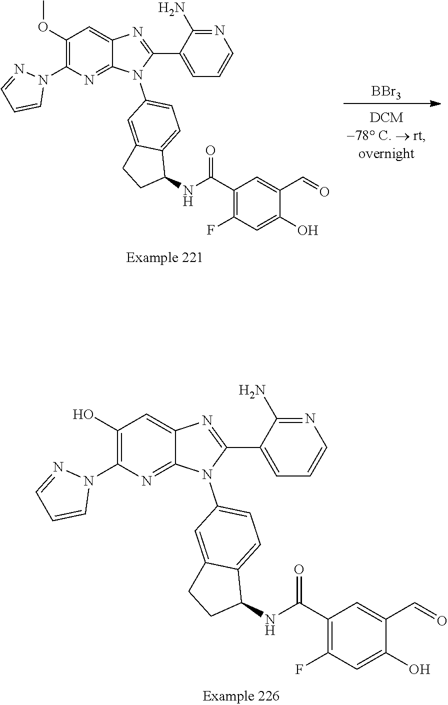 [SMILES: COc1cc2nc(-c3cccnc3N)n(-c3ccc4c(c3)CC[C@@H]4NC(=O)c3cc(C=O)c(O)cc3F)c2nc1-n1cccn1 ; "BBr3" ; Nc1ncccc1-c1nc2cc(O)c(-n3cccn3)nc2n1-c1ccc2c(c1)CC[C@@H]2NC(=O)c1cc(C=O)c(O)cc1F]

### Step 1: N-[(1S)-5-[2-(2-aminopyridin-3-yl)-6-hydroxy-5-(pyrazol-1-yl)imidazo[4,5-b]pyridin-3-yl]-2,3-dihydro-1H-inden-1-yl]-2-fluoro-5-formyl-4-hydroxybenzamide

To a cooled (−78° C.) solution of N-[(1S)-5-[2-(2-aminopyridin-3-yl)-6-methoxy-5-(pyrazol-1-yl)imidazo[4,5-b]pyridin-3-yl]-2,3-dihydro-1H-inden-1-yl]-2-fluoro-5-formyl-4-hydroxybenzamide (Example 221) (60 mg, 0.099 mmol, 1 equiv) in dichloromethane (3 mL) was added BBr₃ (0.49 mL, 0.49 mmol, 1 M in dichloromethane, 5 equiv) and the resulting mixture was stirred overnight at room temperature. The reaction was quenched with ice water at room temperature and the mixture was concentrated to dryness under reduced pressure. The residue obtained was dissolved in N,N-dimethylformamide (4 mL). The crude product was purified by preparative HPLC on a XSelect CSH Fluoro Phenyl column using a 22-45% gradient of acetonitrile in water (+0.1% formic acid) to afford N-[(1S)-5-[2-(2-aminopyridin-3-yl)-6-hydroxy-5-(pyrazol-1-yl)imidazo[4,5-b]pyridin-3-yl]-2,3-dihydro-1H-inden-1-yl]-2-fluoro-5-formyl-4-hydroxybenzamide (12.6 mg, 21%) as a yellow solid. MS(ESI) calcd. for C₃₁H₂₃FN₈O₄: 590.18 m/z, found 591.10 [M+H]⁺. ¹H NMR (400 MHz, DMSO-d₆) δ (ppm): 10.00-10.35 (m, 1H), 8.20-8.55 (m, 1H), 7.90-8.15 (m, 2H), 7.70-7.90 (m, 2H), 7.00-7.50 (m, 4H), 6.70-6.95 (m, 1H), 6.35-6.65 (m, 2H), 5.35-5.80 (m, 1H), 2.75-3.10 (m, 2H), 2.40-2.50 (m, 1H), 1.85-2.05 (m, 1H).

### Example 227: N-[(1S)-5-[2-(2-aminopyridin-3-yl)-7-methyl-5-(1,2,3-triazol-2-yl)imidazo[4,5-b]pyridin-3-yl]-2,3-dihydro-1H-inden-1-yl]-2,3-difluoro-5-formyl-4-hydroxybenzamide

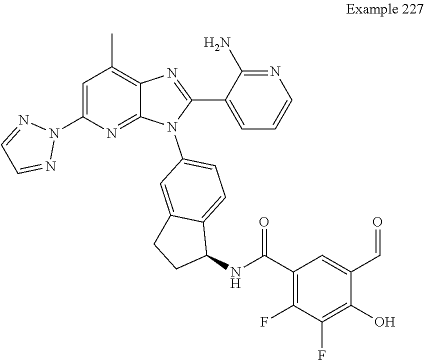 [SMILES: Cc1cc(-n2nccn2)nc2c1nc(-c1cccnc1N)n2-c1ccc2c(c1)CC[C@@H]2NC(=O)c1cc(C=O)c(O)c(F)c1F]

Example 227 was prepared in a manner analogous to Example 7 using Intermediate 227-2 in place of Intermediate 1-2 and Intermediate 118-1 in place of Intermediate 5-1. MS (ESI) calcd. for C₃₁H₂₃F₂N₉O₃: 607.19 m/z, found 608.10 [M+H]⁺. ¹H NMR (400 MHz, DMSO-d₆) δ (ppm): 9.90-10.50 (m, 1H), 8.02-8.15 (m, 2H), 7.90-8.02 (m, 1H), 7.85-7.90 (m, 1H), 7.60-7.85 (m, 1H), 7.25-7.45 (m, 4H), 6.35-6.65 (m, 1H), 5.45-5.65 (m, 1H), 2.90-3.10 (m, 1H), 2.80-2.90 (m, 1H), 2.75-2.80 (m, 3H), 2.48-2.50 (m, 1H), 1.90-2.10 (m, 1H).

### Intermediate 227-2: (S)-3-(3-(1-amino-2,3-dihydro-1H-inden-5-yl)-7-methyl-5-(2H-1,2,3-triazol-2-yl)-3H-imidazo[4,5-b]pyridin-2-yl)pyridin-2-amine

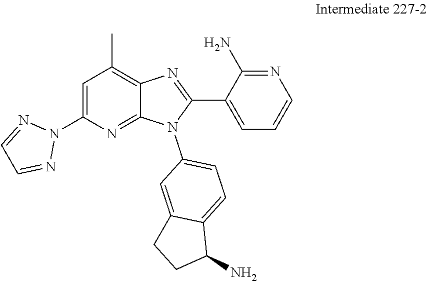 [SMILES: Cc1cc(-n2nccn2)nc2c1nc(-c1cccnc1N)n2-c1ccc2c(c1)CC[C@@H]2N]

Intermediate 227-2 was prepared in a manner analogous to Intermediate 13-2 (starting from Step 2) using Intermediate 227-1 in place of the nitro starting material. MS (ESI) calcd. for C₂₃H₂₁N₉: 423.19 m/z, found 424.20 [M+H]⁺.

### Intermediate 227-1: (S)—N-(5-((4-methyl-3-nitro-6-(2H-1,2,3-triazol-2-yl)pyridin-2-yl)amino)-2,3-dihydro-1H-inden-1-yl)acetamide

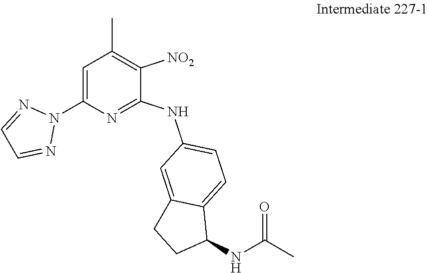 [SMILES: CC(=O)N[C@H]1CCc2cc(Nc3nc(-n4nccn4)cc(C)c3[N+](=O)[O-])ccc21]

### Synthetic Route:

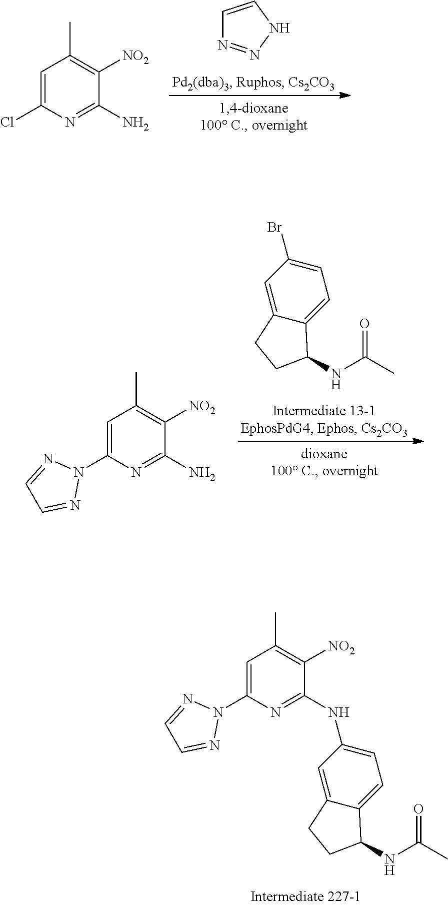 [SMILES: Cc1cc(Cl)nc(N)c1[N+](=O)[O-] ; c1c[nH]nn1 ; Cc1cc(-n2nccn2)nc(N)c1[N+](=O)[O-] ; CC(=O)N[C@H]1CCc2cc(Br)ccc21 ; CC(=O)N[C@H]1CCc2cc(Nc3nc(-n4nccn4)cc(C)c3[N+](=O)[O-])ccc21]

### Step 1: 4-methyl-3-nitro-6-(1,2,3-triazol-2-yl)pyridin-2-amine

To a mixture of 6-chloro-4-methyl-3-nitropyridin-2-amine (2.00 g, 10.7 mmol, 1 equiv) in 1,4-dioxane (40 mL) was added Pd₂(dba)₃ (0.98 g, 1.1 mmol, 0.1 equiv), RuPhos (1.00 g, 2.13 mmol, 0.2 equiv), Cs₂CO₃ (6.95 g, 21.3 mmol, 2 equiv) and 1,2,3-triazole (3.68 g, 53.3 mmol, 5 equiv) under nitrogen atmosphere and the resulting mixture was stirred overnight at 100° C. under nitrogen atmosphere. The reaction was cooled to room temperature and quenched with H₂O (200 mL). The resulting mixture was extracted with ethyl acetate (3×200 mL). The combined organic layers were washed with brine (3×200 mL), dried over anhydrous sodium sulfate, filtered and concentrated under reduced pressure. The residue was purified by silica gel column chromatography using a 25-35% gradient of ethyl acetate in dichloromethane to afford 4-methyl-3-nitro-6-(1,2,3-triazol-2-yl)pyridin-2-amine (2.10 g, 89%) as a green solid. Note that some of the N1 triazole isomer was also obtained and did not separate at this stage. The undesired isomer was purged gradually over the subsequent several steps. MS (ESI) calcd. for C₈H₈N₆O₂: 220.07 m/z, found 221.00 [M+H]⁺.

### Step 2: N-[(1S)-5-{[4-methyl-3-nitro-6-(1,2,3-triazol-2-yl)pyridin-2-yl]amino}-2,3-dihydro-1H-inden-1-yl]acetamide (Intermediate 227-1)

To a stirred mixture of 4-methyl-3-nitro-6-(1,2,3-triazol-2-yl)pyridin-2-amine (2.5 g, 11.4 mmol, 1 equiv) and N-[(1S)-5-bromo-2,3-dihydro-1H-inden-1-yl]acetamide (Intermediate 13-1) (3.46 g, 13.6 mmol, 1.2 equiv) in 1,4-dioxane (75 mL) were added EPhos (1.21 g, 2.27 mmol, 0.2 equiv), EPhos Pd G4 (1.04 g, 1.14 mmol, 0.1 equiv) and Cs₂CO₃ (7.40 g, 22.7 mmol, 2 equiv) under nitrogen atmosphere. The resulting mixture was stirred overnight at 100° C. under nitrogen atmosphere. The reaction mixture was cooled to room temperature and quenched with water (200 mL). The resulting mixture was extracted with ethyl acetate (3×200 mL). The combined organic layers were washed with brine (100 mL), dried over anhydrous sodium sulfate, filtered and concentrated under reduced pressure. The residue was purified by reverse-phase flash column chromatography on C18 silica gel using a 30-50% gradient of acetonitrile in water (+0.1% formic acid) to afford N-[(1S)-5-{[4-methyl-3-nitro-6-(1,2,3-triazol-2-yl)pyridin-2-yl]amino}-2,3-dihydro-1H-inden-1-yl]acetamide (Intermediate 227-1) (580 mg, 8%) as a red solid. MS (ESI) calcd. for C₁₉H₁₉N₇O₃: 393.15 m/z, found 394.15 [M+H]⁺.

### Example 228: N-[(1S)-5-[2-(2-aminopyridin-3-yl)-5-(pyrazol-1-yl)imidazo[4,5-b]pyridin-3-yl]-2,3-dihydro-1H-inden-1-yl]-3-(dimethylamino)-5-formyl-4-hydroxybenzamide

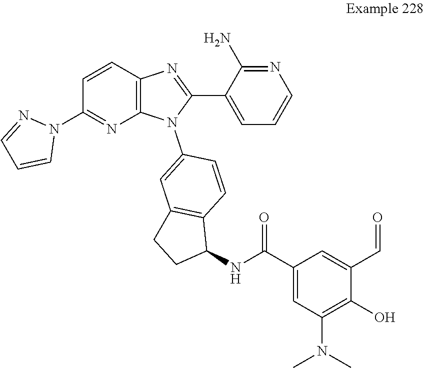 [SMILES: CN(C)c1cc(C(=O)N[C@H]2CCc3cc(-n4c(-c5cccnc5N)nc5ccc(-n6cccn6)nc54)ccc32)cc(C=O)c1O]

Example 228 was prepared in a manner analogous to Example 12 using Intermediate 228-2 in place of Intermediate 12-1 and Intermediate 33-1 in place of Intermediate 1-3. MS (ESI) calcd. for C₃₃H₂₉N₉O₃: 599.24 m/z, found 600.30 [M+H]⁺. ¹H-NMR (400 MHz, DMSO-d₆) δ (ppm): 10.15 (s, 1H), 8.30-8.42 (m, 2H), 7.89-8.05 (m, 3H), 7.75-7.85 (m, 1H), 7.65-7.74 (m, 1H), 7.22-7.42 (m, 4H), 6.51-6.60 (m, 1H), 6.45-6.50 (m, 1H), 5.57-5.70 (m, 1H), 3.01-3.15 (m, 1H), 2.85-2.99 (m, 1H), 2.65-2.83 (m, 6H), 2.40-2.60 (m, 1H), 2.02-2.20 (m, 1H).

### Intermediate 228-2: 4-(benzyloxy)-3-(dimethylamino)-5-(1,3-dioxolan-2-yl)benzoic acid

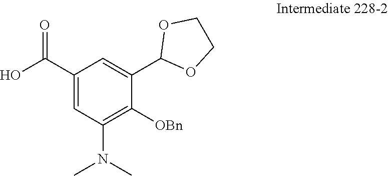 [SMILES: CN(C)c1cc(C(=O)O)cc(C2OCCO2)c1OCc1ccccc1]

Intermediate 228-2 was prepared in a manner analogous to Intermediate 36-1 (starting from Step 2) using Intermediate 228-1 in place of the aldehyde starting material. Following completion of the final step, the reaction mixture was stirred at room temperature for 2 h then quenched with saturated aqueous ammonium chloride (10 mL) and concentrated under vacuum. The obtained material was purified by reverse-phase flash column chromatography on C18 silica gel using a 10-70% gradient of acetonitrile in water (+0.5% 2,2,2-trifluoroacetic acid) to afford 4-(benzyloxy)-3-(dimethylamino)-5-(1,3-dioxolan-2-yl)benzoic acid (Intermediate 228-2) as a white solid. MS (ESI) calcd. for C₁₉H₂₁NO₅: 343.14 m/z, found: 344.15 [M+H]⁺.

### Intermediate 228-1: 2-(benzyloxy)-5-bromo-3-(dimethylamino)benzaldehyde

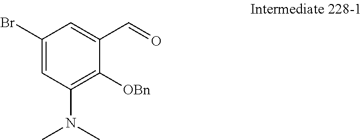 [SMILES: CN(C)c1cc(Br)cc(C=O)c1OCc1ccccc1]

### Synthetic Route:

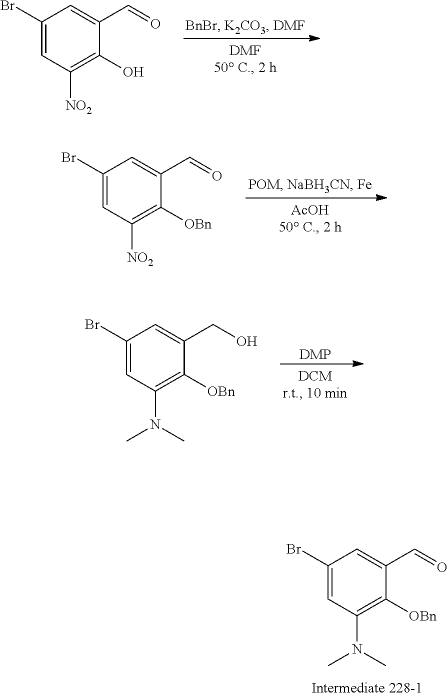

To a solution of 5-bromo-2-hydroxy-3-nitrobenzaldehyde (3.00 g, 12.2 mmol, 1 equiv) in N,N-dimethylformamide (25 mL) was added K₂CO₃ (7.95 g, 24.4 mmol, 2 equiv) and benzyl bromide (3.13 g, 18.3 mmol, 1.5 equiv). The resulting mixture was stirred at 50° C. for 2 h. The mixture was allowed to cool to room temperature. The resulting mixture was diluted with water (200 mL) and extracted with ethyl acetate (3×500 mL). The combined organic layers were washed with brine (200 mL) and dried over anhydrous Na₂SO₄. After filtration, the filtrate was concentrated under reduced pressure. The resulting residue was purified by silica gel column chromatography using a 0-50% gradient of ethyl acetate in petroleum ether to afford 2-(benzyloxy)-5-bromo-3-nitrobenzaldehyde (2.5 g, 61%) as a white solid. MS (ESI) calcd. for C₁₄H₁₀BrNO₄: 334.98 m/z, found 335.90 [M+H]⁺.

### Step 2: Synthesis of [2-(benzyloxy)-5-bromo-3-(dimethylamino)phenyl]methanol

To a solution of 2-(benzyloxy)-5-bromo-3-nitrobenzaldehyde (2.5 g, 7.4 mmol, 1 equiv) in acetic acid (60 mL) was added paraformaldehyde (6.70 g, 74.4 mmol, 10 equiv), NaBH₃CN (0.93 g, 14.9 mmol, 2 equiv) and iron (4.15 g, 74.4 mmol, 10 equiv). The resulting mixture was stirred at 50° C. for 2 h. The reaction was quenched with saturated aqueous ammonium chloride (20 mL). The mixture was neutralized to pH 7 with NaOH. The resulting mixture was extracted with ethyl acetate (2×100 mL). The combined organic layers were washed with H₂O (50 mL), dried over anhydrous sodium sulfate, filtered and concentrated under reduced pressure. The residue was purified by silica gel column chromatography using a 0-50% gradient of ethyl acetate in petroleum ether to afford [2-(benzyloxy)-5-bromo-3-(dimethylamino)phenyl]methanol (515 mg, 20%) as a white oil. MS (ESI) calcd. for C₁₆H₁₈BrNO₂: 335.05 m/z, found: 336.10 [M+H]⁺.

### Step 3: Synthesis of 2-(benzyloxy)-5-bromo-3-(dimethylamino)benzaldehyde (Intermediate 228-1)

To a solution of [2-(benzyloxy)-5-bromo-3-(dimethylamino)phenyl]methanol (500 mg, 1.49 mmol, 1 equiv) in dichloromethane (1 mL) was added Dess-Martin Periodinane (1.26 g, 2.97 mmol, 2 equiv) and the resulting mixture was stirred at room temperature for 10 min. The reaction was quenched with saturated aqueous ammonium chloride (20 mL). The resulting mixture was extracted with ethyl acetate (2×50 mL). The combined organic layers were washed with H₂O (20 mL), dried over anhydrous sodium sulfate, filtered and concentrated under reduced pressure. The residue was purified by silica gel column chromatography using a 0-100% gradient of ethyl acetate in petroleum ether to afford 2-(benzyloxy)-5-bromo-3-(dimethylamino)benzaldehyde (Intermediate 228-1) (372 mg, 75%) as a yellow oil. MS (ESI) calcd. for C₁₆H₁₆BrNO₂: 333.04 m/z, found 334.05 [M+H]⁺.

### Example 229: (S)—N-(5-(2-(2-aminopyridin-3-yl)-6-methyl-5-(1H-pyrazol-1-yl)-3H-imidazo[4,5-b]pyridin-3-yl)-2,3-dihydro-1H-inden-1-yl)-3-formyl-4-hydroxybenzamide

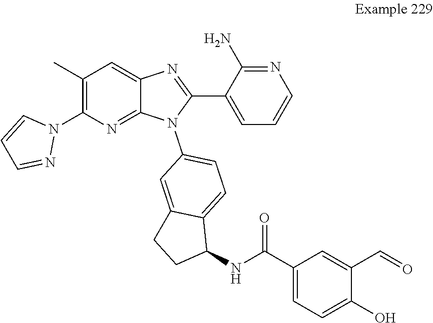 [SMILES: Cc1cc2nc(-c3cccnc3N)n(-c3ccc4c(c3)CC[C@@H]4NC(=O)c3ccc(O)c(C=O)c3)c2nc1-n1cccn1]

Example 229 was prepared in a manner analogous to Example 7 using Intermediate 229-2 in place of Intermediate 1-2 and Intermediate 57-2 in place of Intermediate 5-1. MS (ESI) calcd. for C₃₂H₂₆N₈O₃: 570.21 m/z, found 571.30 [M+H]⁺. ¹H NMR (400 MHz, DMSO-d₆) δ (ppm): 10.26 (s, 1H), 8.26-8.27 (m, 2H), 8.04-8.07 (m, 2H), 8.01-8.02 (m, 1H), 7.74-7.75 (m, 1H), 7.30-7.34 (m, 3H), 7.24-7.26 (m, 1H), 7.05-7.07 (m, 2H), 6.47-6.52 (m, 2H), 5.57-5.61 (m, 1H), 2.98-3.00 (m, 1H), 2.87-2.89 (m, 1H), 2.50-2.51 (m, 1H), 2.38-2.49 (m, 3H), 2.02-2.07 (m, 1H).

### Intermediate 229-2: (S)-3-(3-(1-amino-2,3-dihydro-1H-inden-5-yl)-6-methyl-5-(1H-pyrazol-1-yl)-3H-imidazo[4,5-b]pyridin-2-yl)pyridin-2-amine

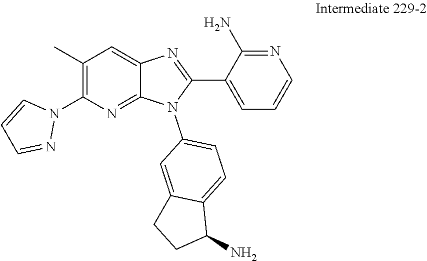 [SMILES: Cc1cc2nc(-c3cccnc3N)n(-c3ccc4c(c3)CC[C@@H]4N)c2nc1-n1cccn1]

Intermediate 229-2 was prepared in a manner analogous to Intermediate 13-1 (starting from Step 2) using Intermediate 229-1 in place of the nitro starting material. MS (ESI) calcd. for C₂₄H₂₂N₈: 422.20 m/z, found 423.20 [M+H]⁺.

### Intermediate 229-1: (S)—N-(5-((5-methyl-3-nitro-6-(1H-pyrazol-1-yl)pyridin-2-yl)amino)-2,3-dihydro-1H-inden-1-yl)acetamide

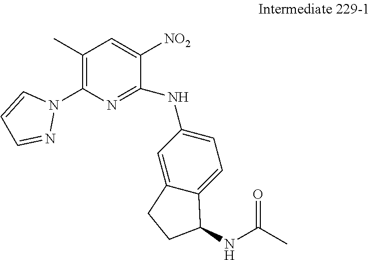 [SMILES: CC(=O)N[C@H]1CCc2cc(Nc3nc(-n4cccn4)c(C)cc3[N+](=O)[O-])ccc21]

### Synthetic Route:

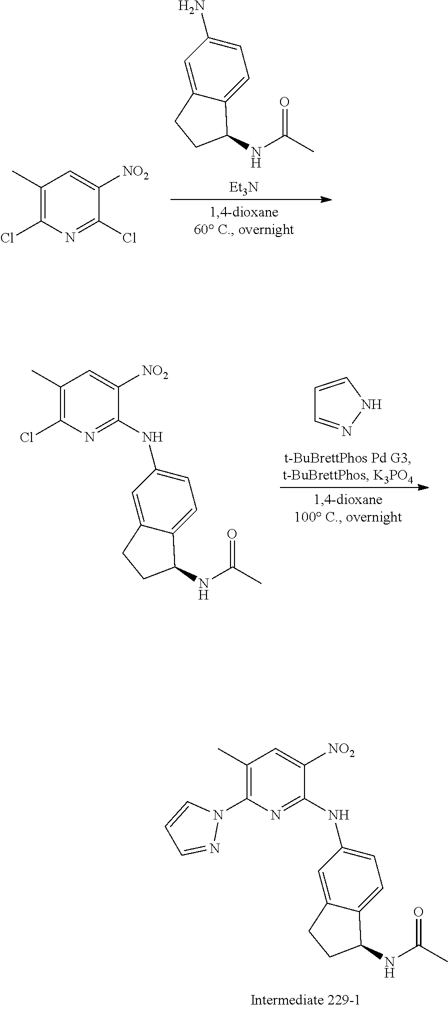 [SMILES: "Et3N" ; CC(=O)N[C@H]1CCc2cc(N)ccc21 ; Cc1cc([N+](=O)[O-])c(Cl)nc1Cl ; CC(=O)N[C@H]1CCc2cc(Nc3nc(Cl)c(C)cc3[N+](=O)[O-])ccc21 ; c1cn[nH]c1 ; CC(=O)N[C@H]1CCc2cc(Nc3nc(-n4cccn4)c(C)cc3[N+](=O)[O-])ccc21]

### Step 1: Synthesis of (S)—N-(5-((6-chloro-5-methyl-3-nitropyridin-2-yl)amino)-2,3-dihydro-1H-inden-1-yl)acetamide

A solution of 2,6-dichloro-3-methyl-5-nitropyridine (2.00 g, 9.66 mmol, 1 equiv) and (S)—N-(5-amino-2,3-dihydro-1H-inden-1-yl)acetamide (1.84 g, 9.661 mmol, 1 equiv) and triethylamine (3.911 mg, 38.64 mmol, 4 equiv) in 1,4-dioxane (60 mL) was stirred at 60° C. overnight. The resulting mixture was poured into 600 mL of water. The precipitated solids were collected by filtration, and then washed with water and petroleum ether/ethyl acetate (1:1) in sequence then dried by air to afford (S)—N-(5-((6-chloro-5-methyl-3-nitropyridin-2-yl)amino)-2,3-dihydro-1H-inden-1-yl)acetamide (1.8 g, 43%) as an orange solid. MS (ESI) calcd. for C₁₇H₁₇ClN₄O₃: 360.10 m/z, found 359.05 [M−H]⁻.

### Step 2: Synthesis of (S)—N-(5-((5-methyl-3-nitro-6-(1H-pyrazol-1-yl)pyridin-2-yl)amino)-2,3-dihydro-1H-inden-1-yl)acetamide (Intermediate 229-1)

To a solution of (S)—N-(5-((6-chloro-5-methyl-3-nitropyridin-2-yl)amino)-2,3-dihydro-1H-inden-1-yl)acetamide (1.3 g, 3.6 mmol, 1 equiv) in 1,4-dioxane (30 mL) were added pyrazole (736 mg, 10.8 mmol, 3 equiv), t-BuBrettPhos Pd G3 (308 mg, 0.360 mmol, 0.1 equiv), t-BuBrettPhos (349 mg, 0.721 mmol, 0.2 equiv) and K₃PO₄ (1.53 mg, 7.21 mmol, 2 equiv). The resulting mixture was stirred at 100° C. overnight under N₂ atmosphere. The reaction was quenched with H₂O (100 mL), and then extracted with ethyl acetate (3×100 mL). The combined organic layers were washed with brine, dried over anhydrous sodium sulfate, filtered and concentrated under reduced pressure. The residue was purified by silica gel column chromatography using a 0-50% gradient of ethyl acetate in petroleum ether to afford (S)—N-(5-((5-methyl-nitro-6-(1H-pyrazol-1-yl)pyridin-2-yl)amino)-2,3-dihydro-1H-inden-1-yl)acetamide (400 mg, 26%) as an orange solid. MS (ESI) calcd. for C₂₀H₂₀N₆O₃: 392.16 m/z, found 393.15 [M+H]⁺.

### Example 230: N-[(1S)-5-[2-(2-aminopyridin-3-yl)-7-cyclopropyl-5-(pyrazol-1-yl)imidazo[4,5-b]pyridin-3-yl]-2,3-dihydro-1H-inden-1-yl]-3-formyl-4-hydroxybenzamide

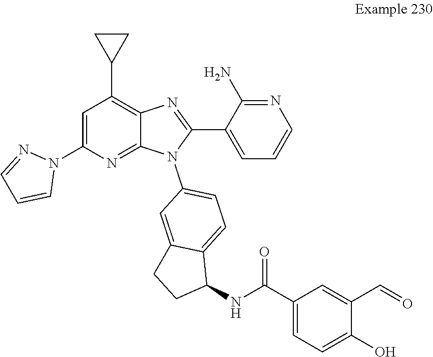 [SMILES: Nc1ncccc1-c1nc2c(C3CC3)cc(-n3cccn3)nc2n1-c1ccc2c(c1)CC[C@@H]2NC(=O)c1ccc(O)c(C=O)c1]

### Synthetic Route:

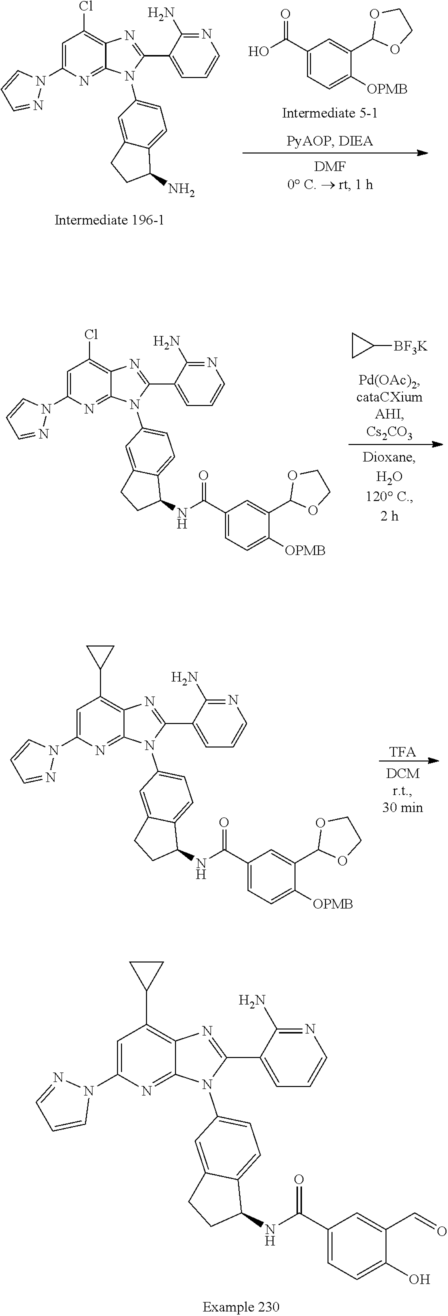

### Step 1: Synthesis of (S)—N-(5-(2-(2-aminopyridin-3-yl)-7-chloro-5-(1H-pyrazol-1-yl)-3H-imidazo[4,5-b]pyridin-3-yl)-2,3-dihydro-1H-inden-1-yl)-3-(1,3-dioxolan-2-yl)-4-((4-methoxybenzyl)oxy)benzamide

To a solution of (S)-3-(3-(1-amino-2,3-dihydro-1H-inden-5-yl)-5-(1H-pyrazol-1-yl)-3H-imidazo[4,5-b]pyridin-2-yl)pyridin-2-amine (Intermediate 196-1) (142 mg, 0.296 mmol, 1 equiv) and 3-(1,3-dioxolan-2-yl)-4-((4-methoxybenzyl)oxy)benzoic acid (Intermediate 5-1) (103 mg, 0.311 mmol, 1.05 equiv) in DMF (2 mL) was added (7-azabenzotriazol-1-yloxy)tripyrrolidinophosphonium hexafluorophosphate (PyAOP) (170 mg, 0.326 mmol, 1.2 equiv) and the mixture was cooled to 0° C. N,N-diisopropylethylamine (160 μL, 0.909 mmol, 3 equiv) was then added and the mixture was allowed to slowly warm to room temperature and stirring was continued for 1 h. The reaction mixture was then quenched with water (5 mL) and extracted with DCM (2×10 mL). The organic layers were combined, washed with brine (5 mL), dried over sodium sulfate and concentrated in vacuo. The residue was purified by silica gel column chromatography (50-100% ethyl acetate in heptanes) to obtain (S)—N-(5-(2-(2-aminopyridin-3-yl)-7-chloro-5-(1H-pyrazol-1-yl)-3H-imidazo[4,5-b]pyridin-3-yl)-2,3-dihydro-1H-inden-1-yl)-3-(1,3-dioxolan-2-yl)-4-((4-methoxy-benzyl)oxy)benzamide (191 mg, 85%) as a yellow oil. MS (ESI) calculated for C₄₁H₃₅ClN₈O₅: 754.24, found 755.19.

### Step 2: Synthesis of N-[(1S)-5-[2-(2-aminopyridin-3-yl)-7-cyclopropyl-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide

A mixture of N-[(1S)-5-[2-(2-aminopyridin-3-yl)-7-chloro-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (80 mg, 0.11 mmol, 1 equiv), potassium cyclopropyltrifluoroborate (19 mg, 0.13 mmol. 1.2 equiv), Pd(OAc)₂ (2.4 mg, 0.011 mmol, 0.1 equiv), Cs₂CO₃ (104 mg, 0.318 mmol, 3 equiv) and bis(adamantan-1-yl)(butyl)phosphane (7.5 mg, 0.021 mmol, 0.2 equiv) was added 1,4-dioxane (1.6 mL) and H₂O (0.4 mL) under N₂. The mixture was stirred at 120° C. for 2 h under nitrogen atmosphere. The reaction was quenched with H₂O (50 mL) at room temperature and extracted with ethyl acetate (3×50 mL). The combined organic layers were washed with brine (50 mL), dried over anhydrous sodium sulfate, filtered and concentrate in vacuo. The resulting mixture was concentrated under reduced pressure. The crude product was purified by silica gel column chromatography using a 0-10% gradient of methanol in dichloromethane to afford N-[(1S)-5-[2-(2-aminopyridin-3-yl)-7-cyclopropyl-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (65 mg, 72%) as a yellow solid. MS (ESI) calcd. for C₄₄H₄₀N₈O₅: 760.31 m/z, found 761.35 [M+H]⁺.

### Step 2: Synthesis of N-[(1S)-5-[2-(2-aminopyridin-3-yl)-7-cyclopropyl-5-(pyrazol-1-yl)imidazo[4,5-b]pyridin-3-yl]-2,3-dihydro-1H-inden-1-yl]-3-formyl-4-hydroxybenzamide (Example 230)

To a solution of N-[(1S)-5-[2-(2-aminopyridin-3-yl)-7-cyclopropyl-5-(pyrazol-1-yl)imidazo[4,5-b]pyridin-3-yl]-2,3-dihydro-1H-inden-1-yl]-3-(1,3-dioxolan-2-yl)-4-[(4-methoxyphenyl)methoxy]benzamide (65 mg, 0.085 mmol, 1 equiv) in dichloromethane (3.75 mL) was added 2,2,2-trifluoroacetic acid (0.25 mL). The resulting mixture was stirred at room temperature for 30 min. The reaction mixture was concentrated under reduced pressure. The resulting crude material was purified by preparative HPLC on a XBridge Shield RP18 OBD Column using a 34-51% gradient of acetonitrile in water (+10 mmol/L ammonium bicarbonate) to afford N-[(1S)-5-[2-(2-aminopyridin-3-yl)-7-cyclopropyl-5-(pyrazol-1-yl)imidazo[4,5-b]pyridin-3-yl]-2,3-dihydro-1H-inden-1-yl]-3-formyl-4-hydroxybenzamide (Example 230) (16.6 mg, 31%) as an off-white solid. MS (ESI) calcd. for C₃₄H₂₈N₈O₃: 596.23 m/z, found 597.30 [M+H]⁺. ¹H NMR (300 MHz, DMSO-d₆) δ (ppm): 10.26 (s, 1H), 8.31-8.32 (m, 1H), 8.18-8.29 (m, 1H), 7.99-8.05 (m, 2H), 7.75-7.77 (m, 1H), 7.51-7.52 (m, 1H), 7.33-7.35 (m, 2H), 7.24-7.26 (m, 2H), 6.88-6.93 (m, 1H), 6.51-6.54 (m, 1H), 6.43-6.45 (m, 1H), 5.59-5.63 (m, 1H), 2.99-3.01 (m, 1H), 2.88-2.90 (m, 1H), 2.60-2.63 (m, 1H), 2.35-2.50 (m, 1H), 2.01-2.11 (m, 1H), 1.22-1.32 (m, 4H).

### Example 231: N-[(1S)-5-[2-(2-aminopyridin-3-yl)-7-(azetidin-1-yl)-5-(pyrazol-1-yl)imidazo[4,5-b]pyridin-3-yl]-2,3-dihydro-1H-inden-1-yl]-3-formyl-4-hydroxybenzamide

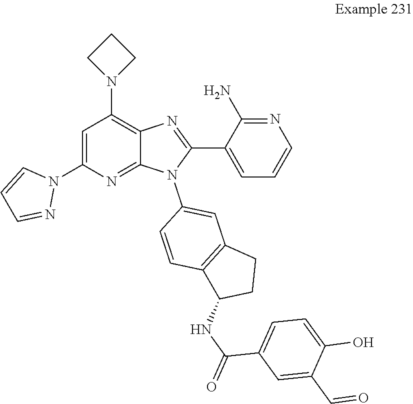 [SMILES: Nc1ncccc1-c1nc2c(N3CCC3)cc(-n3cccn3)nc2n1-c1ccc2c(c1)CC[C@@H]2NC(=O)c1ccc(O)c(C=O)c1]

Example 231 was prepared in a manner analogous to Example 156 (starting from Step 2) using Intermediate 201-1 in place of the chloride starting material and using 4 equivalents of azetidine. MS (ESI) calcd. for C₃₄H₂₉N₉O₃: 611.24 m/z, found 612.25 [M+H]⁺. ¹H NMR (400 MHz, DMSO-d₆) δ (ppm): 10.23-10.24 (m, 1H), 8.24-8.25 (m, 2H), 7.94-7.95 (m, 2H), 7.70-7.71 (m, 1H), 7.29-7.31 (m, 2H), 7.19-7.20 (m, 1H), 7.11-7.13 (m, 1H), 6.90-6.91 (m, 1H), 6.67-6.68 (m, 1H), 6.46-6.47 (m, 1H), 6.39-6.40 (m, 1H), 5.58-5.59 (m, 1H), 4.40-4.41 (m, 4H), 2.99-3.01 (m, 1H), 2.89-2.90 (m, 1H), 2.50-2.51 (m, 1H), 2.02-2.04 (m, 3H).

### Example 232: N-[(1S)-5-[2-(2-aminopyridin-3-yl)-7-methanesulfonamido-5-(pyrazol-1-yl)imidazo[4,5-b]pyridin-3-yl]-2,3-dihydro-1H-inden-1-yl]-3-formyl-4-hydroxybenzamide

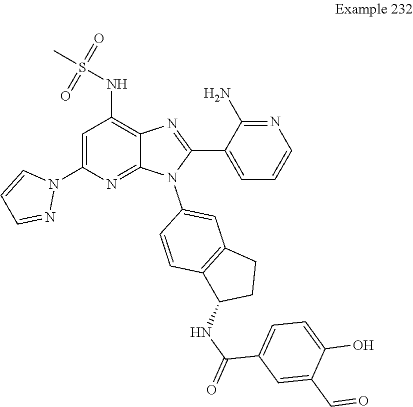 [SMILES: CS(=O)(=O)Nc1cc(-n2cccn2)nc2c1nc(-c1cccnc1N)n2-c1ccc2c(c1)CC[C@@H]2NC(=O)c1ccc(O)c(C=O)c1]

Example 232 was prepared in a manner analogous to Example 156 (starting from Step 2) using Intermediate 197-2 in place of the chloride starting material, methane sulfonamide (1.5 equiv) instead of azetidine and dichloromethane/2,2,2-trifluoroacetic acid (20:9) in place of 2,2,2-trifluoroacetic acid/methanesulfonic acid. MS (ESI) calcd. for C₃₂H₂₇N₉O₅S: 649.19 m/z, found 650.35 [M+H]⁺. ¹H NMR (400 MHz, DMSO-d₆) δ (ppm): 10.32 (s, 1H), 8.95-8.97 (m, 1H), 8.31-8.33 (m, 2H), 8.10-8.13 (m, 1H), 7.99-8.01 (m, 2H), 7.78-7.80 (m, 1H), 7.36-7.38 (m, 3H), 7.25-7.27 (m, 2H), 7.07-7.16 (m, 1H), 6.52-6.53 (m, 1H), 6.36-6.39 (m, 1H), 5.64-5.66 (m, 1H), 3.32-3.34 (m, 3H), 3.05-3.09 (m, 1H), 2.89-2.91 (m, 1H), 2.50-2.51 (m, 1H), 2.30-2.31 (m, 1H).

### Example 233: N-[(1S)-5-[2-(2-aminopyridin-3-yl)-7-(3-oxo-2H-1,2,4-triazol-4-yl)-5-(pyrazol-1-yl)imidazo[4,5-b]pyridin-3-yl]-2,3-dihydro-1H-inden-1-yl]-3-formyl-4-hydroxybenzamide

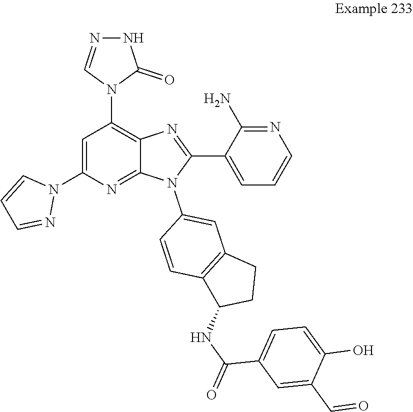 [SMILES: Nc1ncccc1-c1nc2c(-n3cn[nH]c3=O)cc(-n3cccn3)nc2n1-c1ccc2c(c1)CC[C@@H]2NC(=O)c1ccc(O)c(C=O)c1]

Example 233 was prepared in a manner analogous to Example 198 using 2,4-dihydro-1,2,4-triazol-3-one in place of pyrazole. MS (ESI) calcd. for C₃₃H₂₅N₁₁O₄: 639.21 m/z, found 640.20 [M+H]⁺. ¹H NMR (400 MHz, DMSO-d₆) δ (ppm): 10.09-10.32 (m, 1H), 9.05-9.28 (m, 1H), 8.56-8.83 (m, 1H), 8.18-8.46 (m, 2H), 7.91-8.18 (m, 2H), 7.68-7.91 (m, 1H), 7.18-7.49 (m, 4H), 6.98-7.08 (m, 1H), 6.39-6.69 (m, 2H), 5.46-5.71 (m, 1H), 2.98-3.21 (m, 1H), 2.78-2.98 (m, 1H), 2.50-2.51 (m, 1H), 1.96-2.26 (m, 1H).

### Example 234: N-[(1S)-5-[2-(2-aminopyridin-3-yl)-7-methyl-5-(1,2,3-triazol-2-yl)imidazo[4,5-b]pyridin-3-yl]-2,3-dihydro-1H-inden-1-yl]-3-formyl-4-hydroxybenzamide

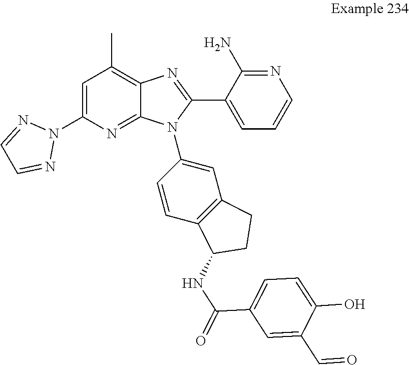 [SMILES: Cc1cc(-n2nccn2)nc2c1nc(-c1cccnc1N)n2-c1ccc2c(c1)CC[C@@H]2NC(=O)c1ccc(O)c(C=O)c1]

Example 234 was prepared in a manner analogous to Example 7 using Intermediate 227-2 in place of Intermediate 1-2 and Intermediate 57-2 in place of Intermediate 5-1. MS (ESI) calcd. for C₃₁H₂₅N₉O₃: 571.21 m/z, found 572.20 [M+H]⁺. ¹H NMR (400 MHz, DMSO-d₆) δ (ppm): 10.15-10.45 (m, 1H), 8.20-8.35 (m, 1H), 8.00-8.15 (m, 3H), 7.95-8.00 (m, 1H), 7.85-7.95 (m, 1H), 7.25-7.45 (m, 4H), 6.90-7.10 (m, 1H), 6.40-6.60 (m, 1H), 5.45-5.65 (m, 1H), 2.90-3.10 (m, 1H), 2.80-2.90 (m, 1H), 2.75-2.80 (m, 3H), 2.48-2.50 (m, 1H), 1.95-2.15 (m, 1H).

### Example 235: N-[(1S)-5-[2-(2-aminopyridin-3-yl)-5-(pyrazol-1-yl)imidazo[4,5-b]pyridin-3-yl]-2,3-dihydro-1H-inden-1-yl]-2-azido-5-formyl-4-hydroxybenzamide

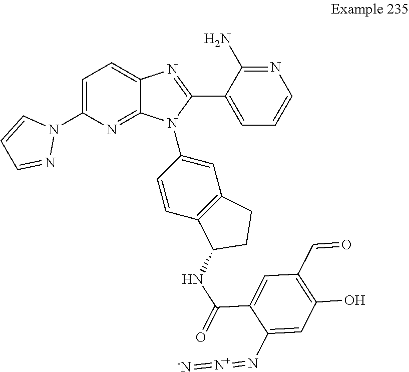

Example 235 was prepared in a manner analogous to Example 12 using Intermediate 33-1 in place of Intermediate 1-3 and Intermediate 235-2 in place of Intermediate 12-1. MS (ESI) calcd. for C₃₁H₂₃N₁₁O₃: 597.20 m/z, found 598.10 [M+H]⁺. ¹H-NMR (400 MHz, DMSO-d₆) δ (ppm): 10.12 (s, 1H), 8.38-8.42 (m, 1H), 8.32-8.37 (m, 1H), 7.78-8.08 (m, 2H), 7.88-7.95 (m, 1H), 7.75-7.80 (m, 2H), 7.40-7.45 (m, 2H), 7.28-7.38 (m, 1H), 6.78-6.85 (m, 2H), 6.50-6.58 (m, 1H), 5.45-5.55 (m, 1H), 2.95-3.08 (m, 1H), 2.80-2.94 (m, 1H), 2.45-2.58 (m, 1H), 1.95-2.10 (m, 1H).

### Intermediate 235-2: 2-azido-4-(benzyloxy)-5-(1,3-dioxolan-2-yl)benzoic acid

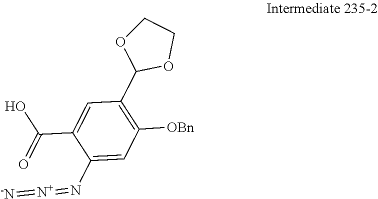

Intermediate 235-2 was prepared in a manner analogous to Intermediate 93-1 (starting from Step 3) using Intermediate 235-1 in place of the aldehyde starting material. MS (ESI) calcd. for C₁₇H₁₅N₃O₅: 341.10 m/z, found 342.09 [M+H]⁺.

### Intermediate 235-1: methyl 2-azido-4-(benzyloxy)-5-formylbenzoate

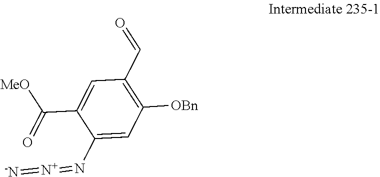

### Synthetic Route:

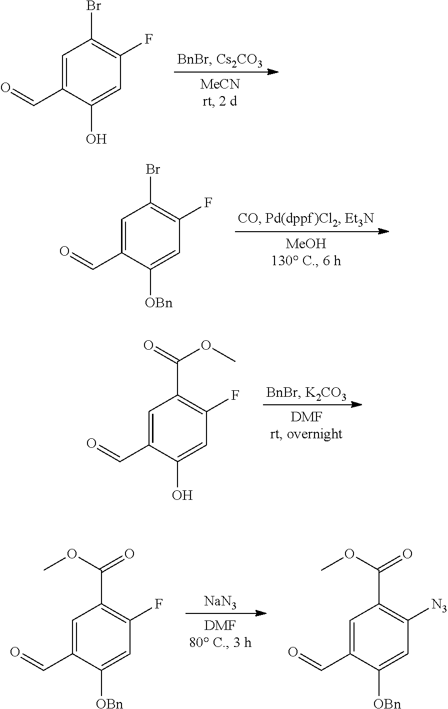

### Step 1: 2-(benzyloxy)-5-bromo-4-fluorobenzaldehyde

To a mixture of 5-bromo-4-fluoro-2-hydroxybenzaldehyde (300 g, 1.37 mol) and Cs₂CO₃ (1.34 kg, 4.10 mol, 3 equiv) in acetonitrile (3 L) was added benzyl bromide (351.43 g, 2.055 mol, 1.5 equiv) and the resulting mixture was stirred overnight at room temperature. The resulting mixture was filtered, rinsing with ethyl acetate (500 mL). The filtrate was concentrated under reduced pressure. The crude product was re-crystallized from petroleum ether (500 mL) to afford 2-(benzyloxy)-5-bromo-4-fluorobenzaldehyde (320 g, 76%) as a white solid. ¹H-NMR (400 MHz, DMSO-d₆) δ (ppm): 10.24 (s, 1H), 7.80-8.00 (m, 1H), 7.48-7.60 (m, 3H), 7.25-7.48 (m, 3H), 5.15-5.45 (m, 2H).

### Step 2: methyl 2-fluoro-5-formyl-4-hydroxybenzoate

To a solution of 2-(benzyloxy)-5-bromo-4-fluorobenzaldehyde (320 g, 1.04 mol, 1 equiv) in methanol (7.5 L) was added [1,1′-bis(diphenylphosphino)ferrocene]dichloropalladium(II) (30.0 g, 36.8 mmol, 0.35 equiv) and triethylamine (314 g, 3.11 mol, 3 equiv) and the reaction mixture was stirred for 6 h at 130° C. under 20 atm of carbon monoxide. The resulting mixture was allowed to cool to room temperature, depressurized and concentrated under vacuum. The residue was acidified to pH 3 with HCl (2M). The resulting mixture was extracted with ethyl acetate (2 L×3). The combined organic layers were dried over anhydrous sodium sulfate, filtered and concentrated under reduced pressure. The residue obtained was purified by silica gel column chromatography using a 0-20% gradient of ethyl acetate/dichloromethane (1:1) in petroleum ether. The crude product was re-crystallized from petroleum ether (300 mL) to afford methyl 2-fluoro-5-formyl-4-hydroxybenzoate (118 g, 57%) as an off-white solid. MS (ESI) calcd. for C₉H₇FO₄: 198.03 m/z, found 197.05 [M−H]⁻.

### Step 3: methyl 4-(benzyloxy)-2-fluoro-5-formylbenzoate

To a mixture of methyl 2-fluoro-5-formyl-4-hydroxybenzoate (118 g, 596 mmol, 1 equiv) and K₂CO₃ (165.8 g, 1.191 mol, 2 equiv) in N,N-dimethylformamide (500 mL) was added (bromomethyl)benzene (152.78 g, 893.27 mmol, 1.5 equiv) and the resulting mixture was stirred overnight at room temperature. The resulting mixture was diluted with ethyl acetate (2 L), washed with water (1.2 L×4), dried over anhydrous sodium sulfate, filtered and concentrated under reduced pressure. The crude product was re-crystallized from petroleum ether (500 mL) and further purified by silica gel column chromatography using a 0-20% gradient of ethyl acetate/dichloromethane (1:1) in petroleum ether to obtain methyl 4-(benzyloxy)-2-fluoro-5-formylbenzoate (111 g, 65%) as a white solid. MS (ESI) calcd. for C₁₆H₁₃N₃O₄: 311.09 m/z, found 312.10 [M+H]⁺.

### Step 4: Synthesis of methyl 2-azido-4-(benzyloxy)-5-formylbenzoate (Intermediate 235-1)

To a solution of methyl 4-(benzyloxy)-2-fluoro-5-formylbenzoate (1.00 g, 3.47 mmol, 1 equiv) in N,N-dimethylformamide (10 mL) was added NaN₃ (0.34 g, 5.2 mmol. 1.5 equiv) and the resulting mixture was stirred at 80° C. for 3 h. The reaction was quenched with water (100 mL) and the precipitated solids were collected by filtration and dried by air to obtain methyl 2-azido-4-(benzyloxy)-5-formylbenzoate (Intermediate 235-1) (800 mg, 74%) as an off-white solid. MS (ESI) calcd. for C₁₆H₁₃N₃O₄: 311.09 m/z, found 312.00 [M+H]⁺.

### Example 236: N-[(1S)-5-[2-(2-aminopyridin-3-yl)-5-(pyrazol-1-yl)imidazo[4,5-b]pyridin-3-yl]-2,3-dihydro-1H-inden-1-yl]-2-fluoro-5-formyl-4-methoxybenzamide

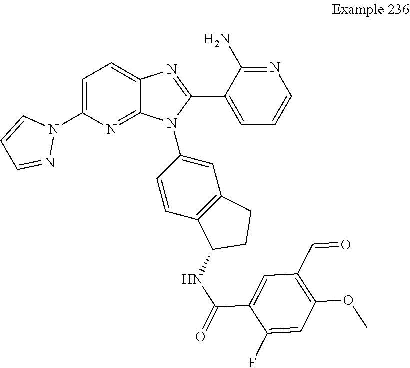 [SMILES: COc1cc(F)c(C(=O)N[C@H]2CCc3cc(-n4c(-c5cccnc5N)nc5ccc(-n6cccn6)nc54)ccc32)cc1C=O]

Example 236 was prepared in a manner analogous to Example 7 using Intermediate 33-1 in place of Intermediate 1-2 and Intermediate 236-2 in place of Intermediate 5-1. MS (ESI) calcd. for C₃₂H₂₅FN₈O₃: 588.20 m/z, found 589.15 [M+H]⁺. ¹H NMR (400 MHz, DMSO-d₆) δ (ppm): 10.26 (s, 1H), 8.22-8.51 (m, 2H), 8.02-8.19 (m, 2H), 7.94-8.02 (m, 1H), 7.79-7.93 (m, 1H), 7.39-7.51 (m, 2H), 7.09-7.39 (m, 3H), 6.51-6.72 (m, 1H), 6.35-6.51 (m, 1H), 5.49-5.76 (m, 1H), 3.97-4.15 (m, 3H), 2.79-3.29 (m, 2H), 2.50-2.52 (m, 1H), 1.90-2.20 (m, 1H). ¹⁹F NMR (376 MHz, DMSO-d₆) δ (ppm): −100.11.

### Intermediate 236-2: 5-(1,3-dioxolan-2-yl)-2-fluoro-4-methoxybenzoic acid

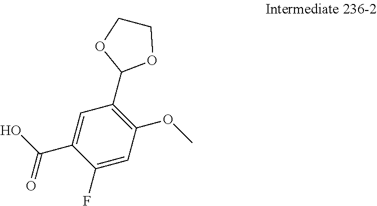 [SMILES: COc1cc(F)c(C(=O)O)cc1C1OCCO1]

Intermediate 236-2 was prepared in a manner analogous to Intermediate 93-1 (starting from Step 3) using Intermediate 236-1 in place of the aldehyde starting material. MS (ESI) calcd. for C₁₁H₁₁FO₅: 242.06 m/z, found 243.10 [M+H]⁺.

### Intermediate 236-1

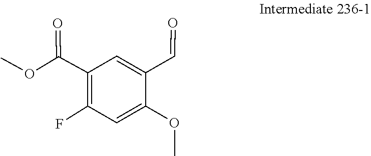

### Synthetic Route:

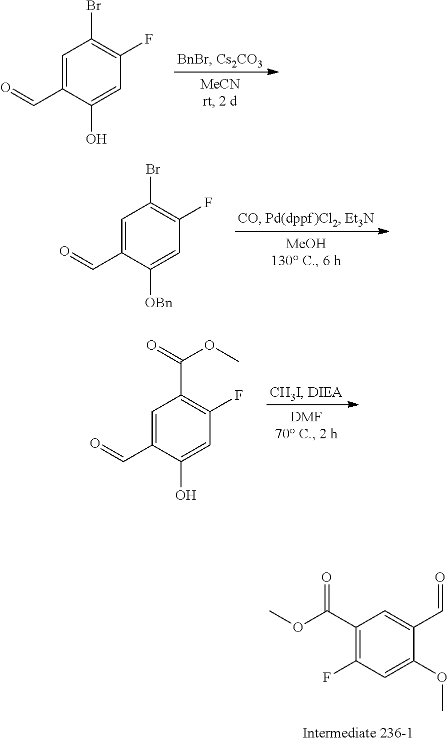

### Step 1: 2-(benzyloxy)-5-bromo-4-fluorobenzaldehyde

To a mixture of 5-bromo-4-fluoro-2-hydroxybenzaldehyde (300 g, 1.37 mol) and Cs₂CO₃ (1.34 kg, 4.10 mol, 3 equiv) in acetonitrile (3 L) was added benzyl bromide (351.43 g, 2.055 mol, 1.5 equiv) and the resulting mixture was stirred overnight at room temperature. The resulting mixture was filtered, rinsing with ethyl acetate (500 mL). The filtrate was concentrated under reduced pressure. The crude product was re-crystallized from petroleum ether (500 mL) to afford 2-(benzyloxy)-5-bromo-4-fluorobenzaldehyde (320 g, 76%) as a white solid. ¹H-NMR (400 MHz, DMSO-d₆) δ (ppm): 10.24 (s, 1H), 7.80-8.00 (m, 1H), 7.48-7.60 (m, 3H), 7.25-7.48 (m, 3H), 5.15-5.45 (m, 2H).

### Step 2: methyl 2-fluoro-5-formyl-4-hydroxybenzoate

To a solution of 2-(benzyloxy)-5-bromo-4-fluorobenzaldehyde (320 g, 1.04 mol, 1 equiv) in methanol (7.5 L) was added [1,1′-bis(diphenylphosphino)ferrocene]dichloropalladium(II) (30.0 g, 36.8 mmol, 0.35 equiv) and triethylamine (314 g, 3.11 mol, 3 equiv) and the reaction mixture was stirred for 6 h at 130° C. under 20 atm of carbon monoxide. The resulting mixture was allowed to cool to room temperature, depressurized and concentrated under vacuum. The residue was acidified to pH 3 with HCl (2M). The resulting mixture was extracted with ethyl acetate (2 L×3). The combined organic layers were dried over anhydrous sodium sulfate, filtered and concentrated under reduced pressure. The residue obtained was purified by silica gel column chromatography using a 0-20% gradient of ethyl acetate/dichloromethane (1:1) in petroleum ether. The crude product was re-crystallized from petroleum ether (300 mL) to afford methyl 2-fluoro-5-formyl-4-hydroxybenzoate (118 g, 57%) as an off-white solid. MS (ESI) calcd. for C₉H₇FO₄: 198.03 m/z, found 197.05 [M−H]⁻.

### Step 3: methyl 2-fluoro-5-formyl-4-methoxybenzoate (Intermediate 236-1)

To a stirred mixture of methyl 2-fluoro-5-formyl-4-hydroxybenzoate (1.00 g, 5.05 mmol, 1 equiv) and K₂CO₃ (1.39 g, 10.1 mmol, 2 equiv) in N,N-dimethylformamide (10 mL) was added CH₃I (1.07 g, 7.57 mmol, 1.5 equiv) in portions at 0° C. The solution was stirred for 2 h at 70° C. The reaction was quenched by the addition of saturated aqueous ammonium chloride (20 mL) at room temperature. The resulting mixture was extracted with ethyl acetate (30×3 mL). The combined organic layers were dried over anhydrous Na₂SO₄. After filtration, the filtrate was concentrated under reduced pressure. The residue was purified by silica gel column chromatography, using a 0-20% gradient of ethyl acetate in petroleum ether to afford methyl 2-fluoro-5-formyl-4-methoxybenzoate (Intermediate 236-1) (775 mg, 70%) as a white solid. MS (ESI) calcd. for C₁₀H₉FO₄: 212.05 m/z, found 213.00 [M+H]⁺.

### Example 237: 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxybenzamide

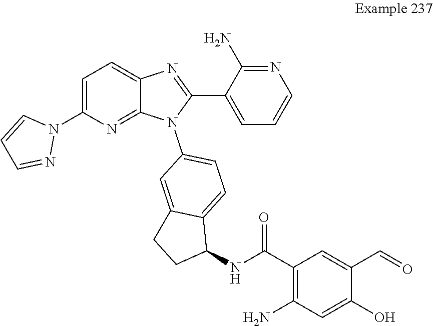 [SMILES: Nc1cc(O)c(C=O)cc1C(=O)N[C@H]1CCc2cc(-n3c(-c4cccnc4N)nc4ccc(-n5cccn5)nc43)ccc21]

### Synthetic Route:

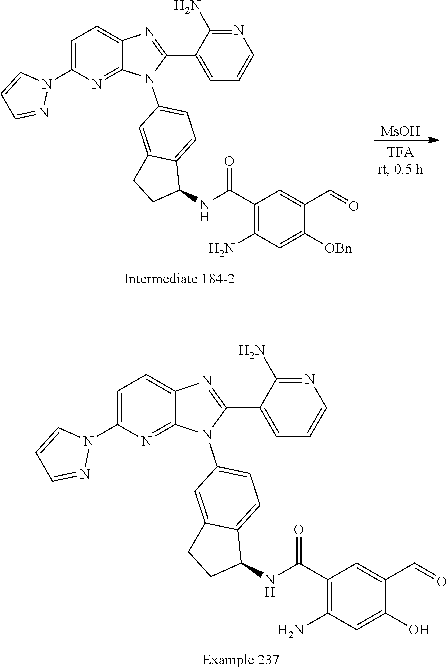 [SMILES: "MsOH" ; Nc1cc(OCc2ccccc2)c(C=O)cc1C(=O)N[C@H]1CCc2cc(-n3c(-c4cccnc4N)nc4ccc(-n5cccn5)nc43)ccc21 ; Nc1cc(O)c(C=O)cc1C(=O)N[C@H]1CCc2cc(-n3c(-c4cccnc4N)nc4ccc(-n5cccn5)nc43)ccc21]

### Step 1: Synthesis of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxybenzamide (Example 237)

To a solution of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-formylbenzamide (Intermediate 184-2) (50 mg, 0.076 mmol, 1 equiv) in TFA (2 mL) was added methanesulfonic acid (0.4 mL). The resulting mixture was stirred at room temperature for 1 h. The resulting mixture were concentrated to give a brown oil, which was purified by preparative HPLC on a XBridge Prep OBD C18 Column using a 20-36% gradient of acetonitrile in water (+0.05% TFA) to afford 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxybenzamide (9.4 mg, 20%) as a yellow solid. MS (ESI) calcd. for C₃₁H₂₅N₉O₃: 571.21 m/z, found 572.30 [M+H]⁺. ¹H NMR (400 MHz, DMSO-d₆) δ (ppm): 9.69 (s, 1H), 8.41-8.43 (m, 1H), 8.37-8.38 (m, 1H), 7.99-8.07 (m, 3H), 7.82-7.83 (m, 1H), 7.74-7.76 (m, 1H), 7.44-7.45 (m, 1H), 7.32-7.39 (m, 2H), 6.77-6.81 (m, 1H), 6.56-6.57 (m, 1H), 6.15 (s, 1H), 5.54-5.59 (m, 1H), 3.01-3.04 (m, 1H), 2.88-2.92 (m, 1H), 2.50-2.51 (m, 1H), 2.05-2.08 (m, 1H).

### Example 238: 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-2,4-dioxo-1H-quinazoline-6-carbaldehyde

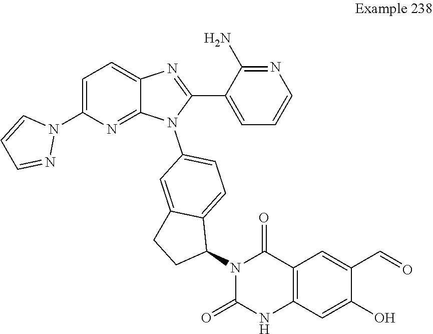 [SMILES: Nc1ncccc1-c1nc2ccc(-n3cccn3)nc2n1-c1ccc2c(c1)CC[C@@H]2n1c(=O)[nH]c2cc(O)c(C=O)cc2c1=O]

### Synthetic Route:

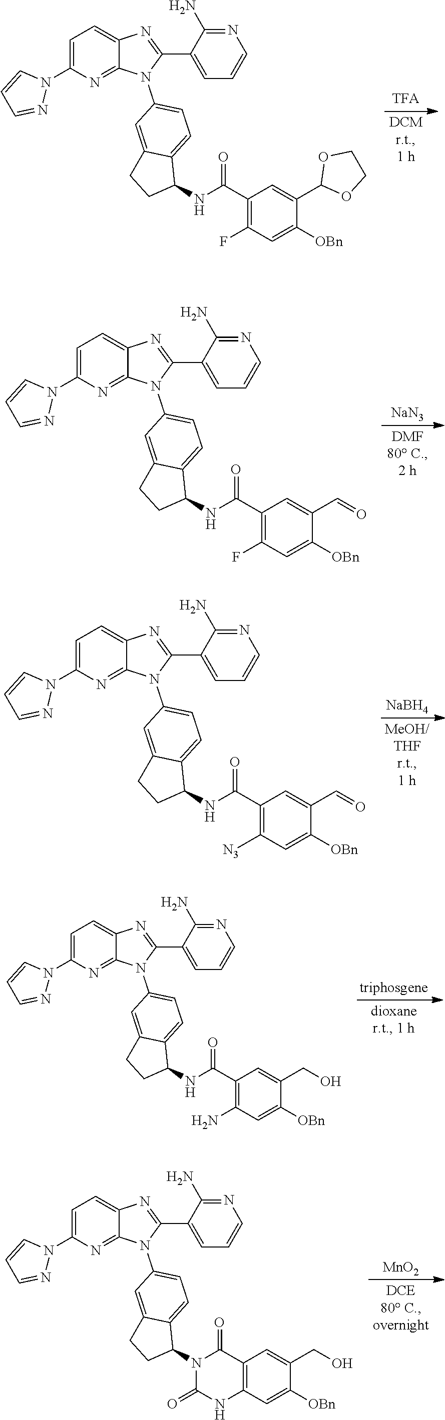

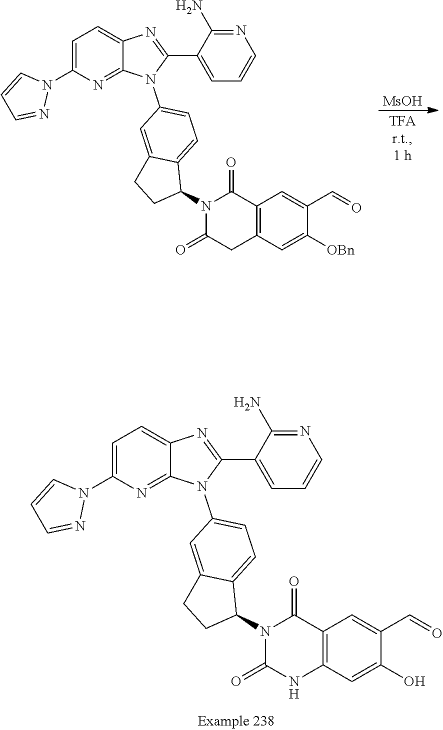 [SMILES: "MsOH" ; Nc1ncccc1-c1nc2ccc(-n3cccn3)nc2n1-c1ccc2c(c1)CC[C@@H]2N1C(=O)Cc2cc(OCc3ccccc3)c(C=O)cc2C1=O ; Nc1ncccc1-c1nc2ccc(-n3cccn3)nc2n1-c1ccc2c(c1)CC[C@@H]2n1c(=O)[nH]c2cc(O)c(C=O)cc2c1=O]

### Step 1: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-2-fluoro-5-formylbenzamide

To a suspension of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-5-(1,3-dioxolan-2-yl)-2-fluorobenzamide (precursor to Example 105) (2.0 g, 2.8 mmol) in DCM (20.0 mL) was added TFA (4.0 mL) and the mixture was stirred at room temperature for 1.0 hours. After concentration, H₂O (20 mL) was added and the mixture was basified to pH 8-9 with saturated aqueous sodium bicarbonate. The mixture was extracted with ethyl acetate (3×100 mL). The combined organic layers were washed with brine (2×100 mL) and dried over anhydrous Na₂SO₄. After filtration, the filtrate was concentrated under reduced pressure to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-2-fluoro-5-formylbenzamide (2.2 g, crude) as a yellow oil, which was used directly in the next step without purification. MS (ESI) calcd. for C₃₈H₂₉FN₈O₃, 664.23 m/z, found: 665.25 [M+H]⁺.

### Step 2: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-azido-4-(benzyloxy)-5-formylbenzamide

To a solution of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-2-fluoro-5-formylbenzamide (2.2 g, crude) in DMF (30.0 mL) was added NaN₃ (200 mg, 3.08 mmol). The resulting solution was stirred for 1 h at room temperature. The reaction was quenched with water (50 mL) and extracted with ethyl acetate (3×100 mL). The combined organic layers were washed with brine and dried over anhydrous Na₂SO₄. After filtration, the filtrate was concentrated under reduced pressure. The obtained residue was purified by reverse-phase flash chromatography on C18 silica gel using a gradient of acetonitrile in water (+0.05% ammonium bicarbonate to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-azido-4-(benzyloxy)-5-formylbenzamide (1.0 g, 51.96 yield) as a white solid. MS (ESI) calcd. for C₃₈H₂₉N₁₁O₃, 687.25 m/z, found 688.25 [M+H]⁺.

### Step 3: Synthesis of (S)-2-amino-N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-5-(hydroxymethyl)benzamide

To a solution of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-azido-4-(benzyloxy)-5-formylbenzamide (1.0 g, 1.46 mmol) in THF (15.0 mL) and MeOH (15.0 mL) was added NaBH₄ (138 mg, 3.64 mmol). The resulting solution was stirred for 1 h at room temperature. After concentration, the residue was purified by reverse-phase flash chromatography on C18 silica gel using a gradient of acetonitrile in water (+0.05% NH₄HCO₃) to afford (S)-2-amino-N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-5-(hydroxymethyl)benzamide (600 mg, 62.17% yield) as a white solid. MS (ESI) calcd. for C₃₈H₃₃N₉O₃, 663.27 m/z, found 664.30 [M+H]⁺.

### Step 4: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-6-(hydroxymethyl)-1H-quinazoline-2,4-dione

To a suspension of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-5-(hydroxymethyl)benzamide (600 mg, 0.904 mmol, 1 equiv) in 1,4-dioxane (10 mL) was added triphosgene (134 mg, 0.452 mmol, 0.5 equiv) and the mixture was stirred at room temperature for 1 hour. After cooling to room temperature, the reaction was concentrated. The residue obtained was purified by silica gel column chromatography using a 0-10% gradient of methanol in dichloromethane to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-6-(hydroxymethyl)-1H-quinazoline-2,4-dione (200 mg, 32%) as a yellow solid. MS (ESI) calcd. for C₃₉H₃₁N₉O₄: 689.25 m/z, found 690.25 [M+H]⁺.

### Step 5: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-2,4-dioxo-1H-quinazoline-6-carbaldehyde

A suspension of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-6-(hydroxymethyl)-1H-quinazoline-2,4-dione (200 mg, 0.290 mmol, 1 equiv) and MnO₂ (1.00 g, 11.5 mmol, 40 equiv) in DCE (10 mL) was stirred at 80° C. overnight. The reaction mixture was cooled to room temperature and filtered. The filtrate was concentrated and the resulting residue was purified by silica gel column chromatography using a 0-10% gradient of methanol in dichloromethane to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-2,4-dioxo-1H-quinazoline-6-carbaldehyde (150 mg, 64%) as a yellow solid. MS (ESI) calcd. for C₃₉H₂₉N₉O₄: 687.23 m/z, found 688.30 [M+H]⁺.

### Step 6: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-2,4-dioxo-1H-quinazoline-6-carbaldehyde (Example 238)

To a solution of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-(benzyloxy)-2,4-dioxo-1H-quinazoline-6-carbaldehyde (100 mg, 0.145 mmol) in 2,2,2-trifluoroacetic acid (1.5 mL) was added methanesulfonic acid (0.3 mL) and the resulting solution was stirred at room temperature for 1 h. The reaction mixture was concentrated in vacuo and the crude material was purified by reverse-phase flash column chromatography on C18 silica gel using a 20-70% gradient of acetonitrile in water (+0.05% TFA) to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-2,4-dioxo-1H-quinazoline-6-carbaldehyde (Example 238) (2,2,2-trifluoroacetic acid salt) as an off-white solid (10.6 mg, 12%). MS (ESI) calcd. for C₃₂H₂₃N₉O₄: 597.19 m/z, found 598.10 [M+H]⁺. ¹H-NMR (400 MHz, DMSO-d₆) δ (ppm): 10.15 (s, 1H), 8.34-8.37 (m, 3H), 8.00-8.01 (m, 1H), 7.94-7.99 (m, 1H), 7.80-7.81 (m, 1H), 7.37 (s, 1H), 7.23-7.25 (m, 1H), 7.19-7.19 (m, 2H), 6.67-6.69 (m, 1H), 6.42-6.56 (m, 3H), 3.19-3.21 (m, 1H), 2.98-3.00 (m, 1H), 2.50-2.51 (m, 1H), 2.34-2.35 (m, 1H).

### Example 239: 5-({[(1S)-5-[2-(2-aminopyridin-3-yl)-5-(pyrazol-1-yl)imidazo[4,5-b]pyridin-3-yl]-2,3-dihydro-1H-inden-1-yl]amino}methyl)-4-fluoro-2-hydroxybenzaldehyde

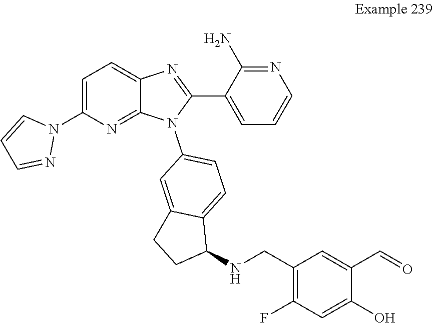 [SMILES: Nc1ncccc1-c1nc2ccc(-n3cccn3)nc2n1-c1ccc2c(c1)CC[C@@H]2NCc1cc(C=O)c(O)cc1F]

### Synthetic Route:

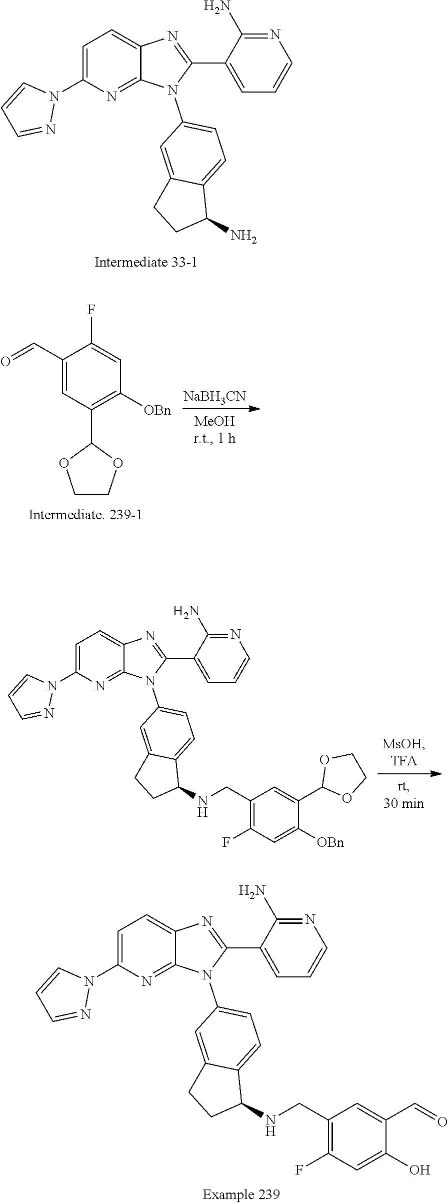

### Step 1: Synthesis of 3-{3-[(1S)-1-({[4-(benzyloxy)-5-(1,3-dioxolan-2-yl)-2-fluorophenyl]methyl}amino)-2,3-dihydro-1H-inden-5-yl]-5-(pyrazol-1-yl)imidazo[4,5-b]pyridin-2-yl}pyridin-2-amine

To a solution of 4-(benzyloxy)-5-(1,3-dioxolan-2-yl)-2-fluorobenzaldehyde (Intermediate 239-1) (200 mg, 0.662 mmol, 1 equiv) and 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (270 mg, 0.662 mmol, 1 equiv) in MeOH (3 mL) was added NaBH₃CN (62 mg, 0.99 mmol, 1.5 equiv). The resulting mixture was stirred at room temperature for 1 h. The reaction mixture was purified by reverse phase flash column chromatography on C18 silica gel using a 5-70% gradient of acetonitrile in water (+0.05% NH₄HCO₃) to afford 3-{3-[(1S)-1-({[4-(benzyloxy)-5-(1,3-dioxolan-2-yl)-2-fluorophenyl]methyl}amino)-2,3-dihydro-1H-inden-5-yl]-5-(pyrazol-1-yl)imidazo[4,5-b]pyridin-2-yl}pyridin-2-amine (300 mg, 65%) as a yellow solid. MS (ESI) calcd. for C₄₀H₃₅FN₈O₃: 694.28 m/z, found 695.30 [M+H]⁺.

### Step 5: Synthesis of 5-({[(1S)-5-[2-(2-aminopyridin-3-yl)-5-(pyrazol-1-yl)imidazo[4,5-b]pyridin-3-yl]-2,3-dihydro-1H-inden-1-yl]amino}methyl)-4-fluoro-2-hydroxybenzaldehyde (Example 239)

To a solution of 3-{3-[(1S)-1-({[4-(benzyloxy)-5-(1,3-dioxolan-2-yl)-2-fluorophenyl]methyl}amino)-2,3-dihydro-1H-inden-5-yl]-5-(pyrazol-1-yl)imidazo[4,5-b]pyridin-2-yl}pyridin-2-amine (100 mg, 0.144 mmol) in 2,2,2-trifluoroacetic acid (1.5 mL) was added methanesulfonic acid (0.3 mL) and the resulting solution was stirred at room temperature for 1 h. After concentration, the mixture was basified to pH-8 with NaHCO₃/H₂O. The resulting mixture was concentrated under reduced pressure and the crude product was then purified by Preparative HPLC on a XSelect CSH Fluoro Phenyl Column using a 6-24% gradient of acetonitrile in water (+0.05% 2,2,2-trifluoroacetic acid) to afford 5-({[(1S)-5-[2-(2-aminopyridin-3-yl)-5-(pyrazol-1-yl)imidazo[4,5-b]pyridin-3-yl]-2,3-dihydro-1H-inden-1-yl]amino}methyl)-4-fluoro-2-hydroxybenzaldehyde (Example 239) (31.7 mg, 39%) as a yellow solid. MS (ESI) calcd. for C₃₁H₂₅FN₈O₂: 560.21 m/z, found 561.35 [M+H]⁺. ¹H NMR (400 MHz, DMSO-d₆) δ (ppm): 10.24-10.26 (m, 1H), 8.43-8.45 (m, 1H), 8.36-8.37 (m, 1H), 8.07-8.09 (m, 1H), 7.97-8.00 (m, 2H), 7.80-7.83 (m, 1H), 7.77-7.84 (m, 1H), 7.66-7.68 (m, 1H), 7.60-7.61 (m, 1H), 7.44-7.46 (m, 1H), 6.87-6.90 (m, 1H), 6.72-6.73 (m, 1H), 6.57-6.58 (m, 1H), 4.96-5.00 (m, 1H), 4.21-4.30 (m, 2H), 3.19-3.23 (m, 1H), 2.99-3.03 (m, 1H), 2.50-2.51 (m, 1H), 2.33-2.34 (m, 1H). ¹⁹F NMR (376 MHz, DMSO-d₆) δ (ppm): −103.86. (2,2,2-trifluoroacetic acid salt).

### Intermediate 239-1: 4-(benzyloxy)-5-(1,3-dioxolan-2-yl)-2-fluorobenzaldehyde

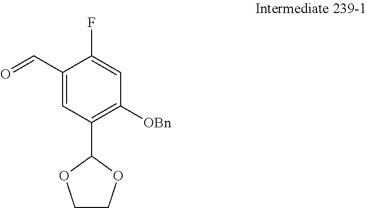 [SMILES: O=Cc1cc(C2OCCO2)c(OCc2ccccc2)cc1F]

### Synthetic Route:

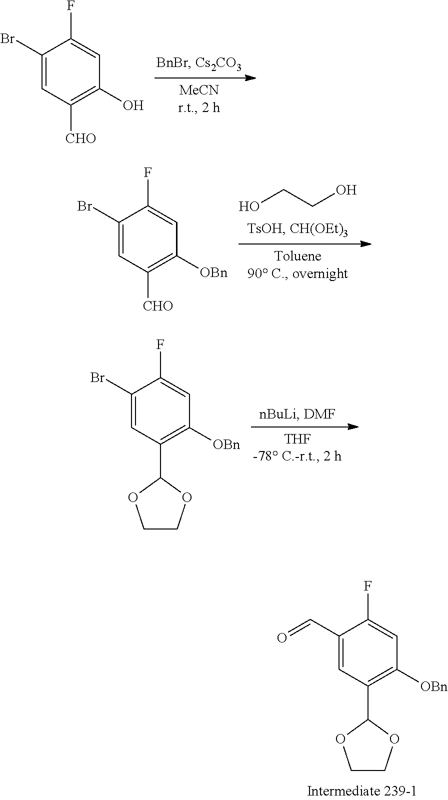

### Step 1: Synthesis of 2-(benzyloxy)-5-bromo-4-fluorobenzaldehyde

A mixture of 5-bromo-4-fluoro-2-hydroxybenzaldehyde (5.00 g, 22.8 mmol, 1 equiv), acetonitrile (100 mL), BnBr (5.86 g, 34.3 mmol, 1.5 equiv) and Cs₂CO₃ (14.88 g, 45.67 mmol, 2 equiv) was stirred at room temperature for 2 h. The reaction was quenched with H₂O (100 mL). The resulting mixture was extracted with DCM (3×100 mL). The organic layers were combined, dried over anhydrous sodium sulfate, filtered and concentrated to obtain 2-(benzyloxy)-5-bromo-4-fluorobenzaldehyde (6 g, 84% yield) as a yellow oil. MS (ESI) calcd. for C₁₄H₁₀BrFO₂: 307.98 m/z, found 307.00 [M−H]⁻.

### Step 2: Synthesis of 2-[2-(benzyloxy)-5-bromo-4-fluorophenyl]-1,3-dioxolane

To a solution of 2-(benzyloxy)-5-bromo-4-fluorobenzaldehyde (6.00 g, 19.4 mmol, 1 equiv) in toluene (70 mL) were added ethylene glycol (7.23 g, 116 mmol, 6 equiv), triethyl orthoformate (8.63 g, 58.2 mmol, 3 equiv) and p-toluenesulfonic acid (334 mg, 1.94 mmol, 0.1 equiv) and the obtained solution was stirred at 90° C. overnight. The resulting mixture was concentrated under reduced pressure and the crude product was purified by silica gel column chromatography using a 0-10% gradient of methanol in dichloromethane to afford 2-[2-(benzyloxy)-5-bromo-4-fluorophenyl]-1,3-dioxolane (5.6 g, 82%) as a yellow oil. MS (ESI) calcd. for C₁₆H₁₄BrFO₃: 352.01 m/z, found 352.95 [M+H]⁺.

### Step 3: Synthesis of 4-(benzyloxy)-5-(1,3-dioxolan-2-yl)-2-fluorobenzaldehyde (Intermediate 239-1)

To a cooled (−78° C.) solution of 2-[2-(benzyloxy)-5-bromo-4-fluorophenyl]-1,3-dioxolane (1.00 g, 2.83 mmol, 1 equiv) in THF (10 mL) was added n-butyllithium (2.81 mL, 7.025 mmol, 2.5 M in hexane, 2.5 equiv) over 10 min and the resulting mixture was stirred for 1 h at −78° C. under N₂ atmosphere. Then to the mixture was added DMF (1 mL) and the mixture was stirred for 1 h at room temperature. The reaction was quenched with H₂O (100 mL). The resulting mixture was extracted with DCM (3×100 mL). The organic layers were combined, dried over anhydrous sodium sulfate, filtered and concentrated. The residue obtained was purified by reverse phase flash column chromatography on C18 silica gel using a 20-60% gradient of acetonitrile in water (+0.05% ammonium bicarbonate) to afford 4-(benzyloxy)-5-(1,3-dioxolan-2-yl)-2-fluorobenzaldehyde (Intermediate 239-1) (500 mg, 58%) as a white solid. MS (ESI) calcd. for C₁₇H₁₅FO₄: 302.10 m/z, found 301.00 [M−H]⁻.

### Example 240: N-[(1S)-5-[2-(2-aminopyridin-3-yl)-5-(1-cyclopropylpyrazol-3-yl)imidazo[4,5-b]pyridin-3-yl]-2,3-dihydro-1H-inden-1-yl]-2-fluoro-5-formyl-4-hydroxybenzamide

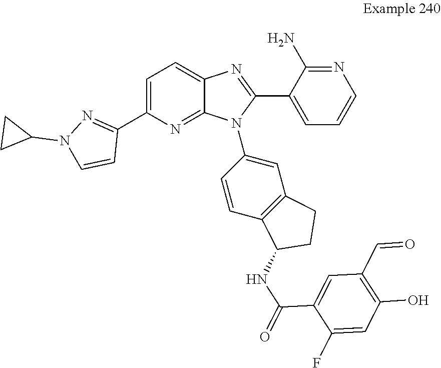 [SMILES: Nc1ncccc1-c1nc2ccc(-c3ccn(C4CC4)n3)nc2n1-c1ccc2c(c1)CC[C@@H]2NC(=O)c1cc(C=O)c(O)cc1F]

Example 240 was prepared in a manner analogous to Example 193 using Intermediate 240-1 in place of Intermediate 193-1 and 3-bromo-1-cyclopropylpyrazole in place of 2-bromo-5-fluoro-1,3-thiazole. MS (ESI) calcd. for C₃₄H₂₇FN₈O₃: 614.22 m/z, found 615.15 [M+H]⁺. ¹H NMR (400 MHz, DMSO-d₆) δ (ppm): 10.01-10.26 (m, 1H), 8.12-8.21 (m, 1H), 7.93-8.12 (m, 3H), 7.69-7.93 (m, 1H), 7.30-7.51 (m, 2H), 7.09-7.31 (m, 2H), 6.75-6.95 (m, 1H), 6.55-6.75 (m, 1H), 6.35-6.55 (m, 1H), 5.49-5.76 (m, 1H), 3.70-4.81 (m, 1H), 2.79-3.19 (m, 2H), 2.55-2.61 (m, 1H), 1.90-2.15 (m, 1H), 0.89-1.21 (m, 4H). ¹⁹F NMR (376 MHz, DMSO-d₆) δ (ppm): −101.74.

### Intermediate 240-1: (S)—N-(5-(2-(2-aminopyridin-3-yl)-5-bromo-3H-imidazo[4,5-b]pyridin-3-yl)-2,3-dihydro-1H-inden-1-yl)-4-(benzyloxy)-5-(1,3-dioxolan-2-yl)-2-fluorobenzamide

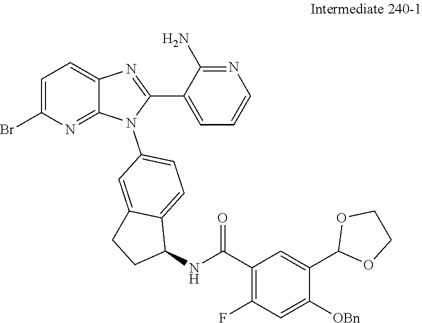 [SMILES: Nc1ncccc1-c1nc2ccc(Br)nc2n1-c1ccc2c(c1)CC[C@@H]2NC(=O)c1cc(C2OCCO2)c(OCc2ccccc2)cc1F]

Intermediate 240-1 was prepared in a manner analogous to Intermediate 94-1 using PyBOP in place of HATU, Intermediate 18-2 in place of Intermediate 79-1 and Intermediate 16-1 in place of Intermediate 57-2. MS (ESI) calcd. for C₃₇H₃₀BrFN₆O₄: 720.15 m/z, found: 721.10 [M+H]⁺.

### Example 241: N-[(1S)-5-[2-(2-aminopyridin-3-yl)-5-(1-methylpyrazol-3-yl)imidazo[4,5-b]pyridin-3-yl]-2,3-dihydro-1H-inden-1-yl]-2-fluoro-5-formyl-4-hydroxybenzamide

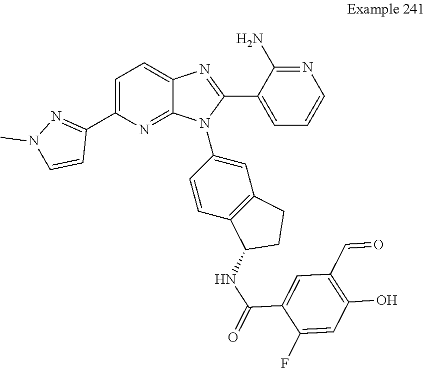 [SMILES: Cn1ccc(-c2ccc3nc(-c4cccnc4N)n(-c4ccc5c(c4)CC[C@@H]5NC(=O)c4cc(C=O)c(O)cc4F)c3n2)n1]

Example 241 was prepared in a manner analogous to Example 19 using 1-methyl-3-(4,4,5,5-tetramethyl-1,3,2-dioxaborolan-2-yl)pyrazole in place of 3-(4-methylpiperazin-1-yl)phenylboronic acid and Intermediate 16-1 in place of Intermediate 5-1. MS (ESI) calcd. for C₃₂H₂₅FN₈O₃: 588.20 m/z, found 589.10 [M+H]⁺. ¹HNMR (400 MHz, DMSO-d₆) δ (ppm): 10.10-10.45 (m, 1H), 8.15-8.35 (m, 1H), 7.85-8.15 (m, 3H), 7.60-7.85 (m, 1H), 7.25-7.60 (m, 4H), 6.75-6.90 (m, 1H), 6.60-6.80 (m, 1H), 6.50-6.60 (m, 1H), 5.55-5.80 (m, 1H), 3.90-4.00 (m, 3H), 2.85-3.10 (m, 2H), 2.40-2.50 (m, 1H), 1.85-2.05 (m, 1H).

### Example 242: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-6-formyl-7-hydroxybenzo[d]thiazole-4-carboxamide

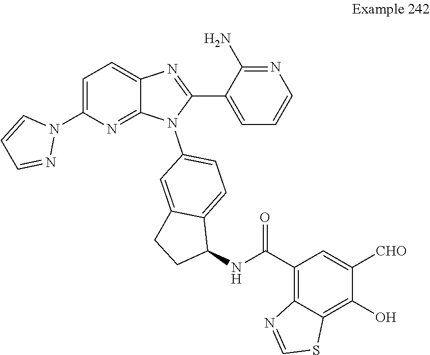 [SMILES: Nc1ncccc1-c1nc2ccc(-n3cccn3)nc2n1-c1ccc2c(c1)CC[C@@H]2NC(=O)c1cc(C=O)c(O)c2scnc12]

Example 242 was prepared in a manner analogous to Example 12 using Intermediate 33-1 in place of Intermediate 1-3 and Intermediate 242-2 in place of Intermediate 12-1. MS (ESI) calcd. for C₃₂H₂₃N₉O₃S: 613.16 m/z, found 614.10 [M+H]⁺. ¹H NMR (300 MHz, DMSO-d₆) δ (ppm): 10.25 (s, 1H), 9.53 (s, 1H), 8.50 (s, 1H), 8.32-8.40 (m, 2H), 7.89-8.02 (m, 2H), 7.85 (s, 1H), 7.40-7.50 (m, 2H), 7.25-7.32 (m, 2H), 6.49-6.58 (m, 2H), 5.60-5.72 (m, 1H), 2.89-3.18 (m, 2H), 2.68-2.80 (m, 1H), 1.95-2.15 (m, 1H).

### Intermediate 242-1: 7-(benzyloxy)-6-(1,3-dioxolan-2-yl)benzo[d]thiazole-4-carboxylic acid

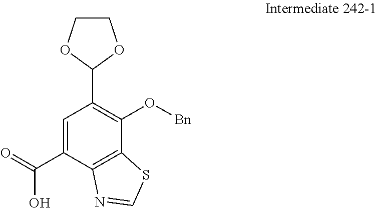 [SMILES: O=C(O)c1cc(C2OCCO2)c(OCc2ccccc2)c2scnc12]

Intermediate 242-1 was prepared in a manner analogous to Intermediate 93-1 starting from Intermediate 242-2 in place of 3-chloro-4-hydroxybenzoic acid. MS (ESI) calcd. for C₁₈H₁₅NO₅S: 357.07 m/z, found 358.00 [M+H]⁺.

### Intermediate 242-2: methyl 7-hydroxybenzo[d]thiazole-4-carboxylate

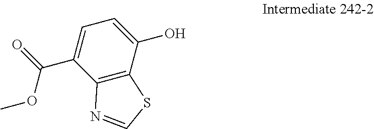

### Synthetic Route:

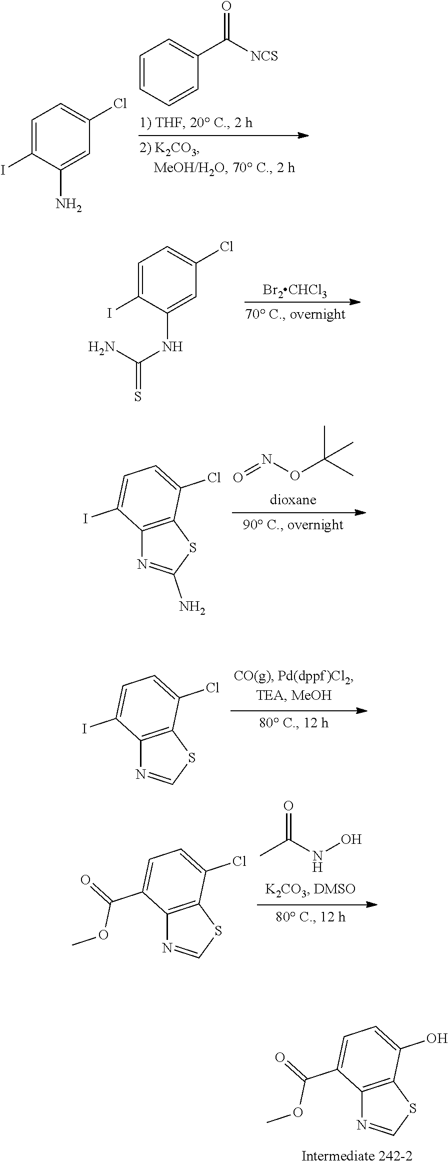

### Step 1: Synthesis of 1-(5-chloro-2-iodophenyl)thiourea

To a solution of 5-chloro-2-iodoaniline (5.00 g, 19.7 mmol) in tetrahydrofuran (100 mL) was added benzoyl isothiocyanate (4.83 g, 29.6 mmol) and the resulting mixture was stirred at room temperature for 2 h. The solvent was removed and the solid was washed with EtOH/petroleum ether (50 mL, v/v=4/1), followed by air-drying. The crude material was dissolved in MeOH (100 mL) and treated with a solution of K₂CO₃ (5.45 g, 39.4 mmol) in H₂O (40 mL). The mixture was stirred at 70° C. for 2 h. The solvent was removed under vacuum followed by addition of water (20 mL). The solid was collected by filtration, washed with water (20 mL), and dried to give 1-(5-chloro-2-iodophenyl)thiourea (1 g, 16%) as a yellow solid. MS (ESI) calcd. for C₇H₆ClIN₂S: 311.90 m/z, found 312.9 [M+H]⁺.

### Step 2: Synthesis of 7-chloro-4-iodobenzo[d]thiazol-2-amine

To a solution of 1-(5-chloro-2-iodophenyl)thiourea (6.00 g, 19.2 mmol) in CHCl₃ (100 mL) was added Br₂ (0.98 mL, 19 mmol) at 0° C. The resulting mixture was stirred overnight at 70° C. After cooling to room temperature, the solvent was removed under vacuum and the solid was dissolved in DCM/MeOH (200 mL, v/v=1/1), followed by addition of aqueous solution of Na₂S₂O₃/H₂O (50 mL). Then the solvent was removed under vacuum and water (100 mL) was added. After filtration, the solid was collected and dried to give 7-chloro-4-iodobenzo[d]thiazol-2-amine (3 g, 31% yield) as a white solid. MS (ESI) calcd. for C₇H₄ClIN₂S: 309.88 m/z, found 310.85 [M+H]⁺.

### Step 3: Synthesis of 7-chloro-4-iodobenzo[d]thiazole

To a solution of 7-chloro-4-iodobenzo[d]thiazol-2-amine (3.00 g, 9.66 mmol) in dioxane (60 mL) was added tert-butyl nitrite (1.99 g, 19.3 mmol) at 0° C. The resulting mixture was stirred overnight at 90° C. The reaction was quenched by the addition of water (200 mL) at room temperature. The resulting mixture was extracted with ethyl acetate (3×200 mL) and dried over anhydrous Na₂SO₄. After filtration, the filtrate was concentrated under reduced pressure. The residue was purified by silica gel column chromatography, eluting with 0-100% ethyl acetate in petroleum ether to afford 7-chloro-4-iodobenzo[d]thiazole (1.8 g, 32% yield) as an off-white solid. MS (ESI) calcd. for C₇H₃ClINS: 294.87 m/z, found: 295.85 [M+H]⁺.

### Step 4: Synthesis of methyl 7-chlorobenzo[d]thiazole-4-carboxylate

To a solution of 7-chloro-4-iodobenzo[d]thiazole (1.8 g, 6.1 mmol) in MeOH (120 mL) was added Pd(dppf)C₁₂ (0.67 g, 0.914 mmol) and triethylamine (1.85 g, 18.3 mmol) in a pressure tank. The mixture was stirred at 80° C. for 12 h under carbon monoxide atmosphere (30 atm). The reaction mixture was cooled to room temperature and filtered to remove solids. The reaction was quenched with water (300 mL). The resulting mixture was extracted with ethyl acetate (3×300 mL). The organic layers were combined, dried over anhydrous Na₂SO₄, filtered and concentrated. The residue obtained was purified by silica gel column chromatography (0-100% ethyl acetate in petroleum ether) to afford methyl 7-chlorobenzo[d]thiazole-4-carboxylate (1.2 g, 81% yield) as a brown solid. MS (ESI) calcd. for C₉H₆ClNO₂S: 226.98 m/z, found 227.90 [M+H]⁺.

### Step 5: Synthesis of methyl 7-hydroxybenzo[d]thiazole-4-carboxylate (Intermediate 242-2)

A mixture of methyl 7-chlorobenzo[d]thiazole-4-carboxylate (1.00 g, 4.39 mmol), N-hydroxyacetamide (2.64 g, 35.1 mmol) and K₂CO₃ (3.035 g, 21.97 mmol) in DMSO (20 mL) was stirred for 12 h at 80° C. After cooling to room temperature, the reaction was quenched with water (200 mL). The resulting mixture was extracted with ethyl acetate (3×100 mL). The organic layers were combined, dried over anhydrous Na₂SO₄, filtered and concentrated. The residue obtained was purified by reverse phase column chromatography on C18 silica gel using a 5-60% gradient of acetonitrile in water (+0.05% NH₄HCO₃) to afford methyl 7-hydroxybenzo[d]thiazole-4-carboxylate (Intermediate 242-2) (350 mg, 34% yield) as an off-white solid. MS (ESI) calcd. for C₉H₇NO₃S: 209.01 m/z, found 210.00 [M+H]⁺.

### Example 243: 3-[(1S)-5-[2-(2-aminopyridin-3-yl)-5-(pyrazol-1-yl)imidazo[4,5-b]pyridin-3-yl]-2,3-dihydro-1H-inden-1-yl]-8-fluoro-7-hydroxy-4-oxoquinazoline-6-carbaldehyde

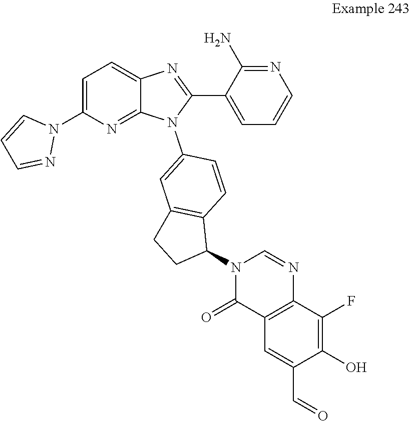 [SMILES: Nc1ncccc1-c1nc2ccc(-n3cccn3)nc2n1-c1ccc2c(c1)CC[C@@H]2n1cnc2c(F)c(O)c(C=O)cc2c1=O]

Example 243 was prepared in a manner analogous to Example 220 using Intermediate 33-1 in place of Intermediate 131-2, Intermediate 243-1 in place of Intermediate 220-1 and a reaction time of 2 h for the final step. MS (ESI) calcd. for C₃₂H₂₂FN₉O₃: 599.18 m/z, found 600.15 [M+H]⁺. ¹H NMR (300 MHz, DMSO-d₆) δ (ppm): 10.29 (s, 1H), 8.38-8.50 (m, 2H), 8.32 (s, 1H), 8.21 (s, 1H), 7.96-8.13 (m, 2H), 7.81 (s, 1H), 7.60-7.73 (m, 1H), 77.55 (s, 1H), 7.26-7.46 (m, 2H), 6.68-6.85 (m, 1H), 6.49-6.63 (m, 1H), 6.20-6.38 (m, 1H), 3.16-3.37 (m, 1H), 2.95-3.15 (m, 1H), 2.63-2.85 (m, 1H), 2.21-2.44 (m, 1H). ¹⁹F NMR (282 MHz, DMSO-d₆) δ (ppm): −148.83. (TFA salt).

### Intermediate 243-1: 2-azido-3-fluoro-5-formyl-4-hydroxybenzoic acid

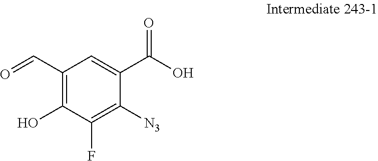 [SMILES: [N-]=[N+]=Nc1c(C(=O)O)cc(C=O)c(O)c1F]

Intermediate 243-1 was prepared in a manner analogous Intermediate 220-1 (starting from Step 3) using Intermediate 243-2 in place of methyl 4-(benzyloxy)-2-fluoro-5-formylbenzoate. Note that the benzyl group is lost on treatment with sodium azide. MS (ESI) calculated for C₈H₄FN₃O₄: 225.02 m/z, found 223.95 [M−H]⁻.

### Intermediate 243-2: methyl 4-(benzyloxy)-2,3-difluoro-5-formylbenzoate

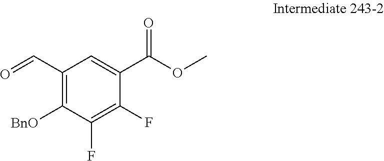 [SMILES: COC(=O)c1cc(C=O)c(OCc2ccccc2)c(F)c1F]

### Synthetic Route:

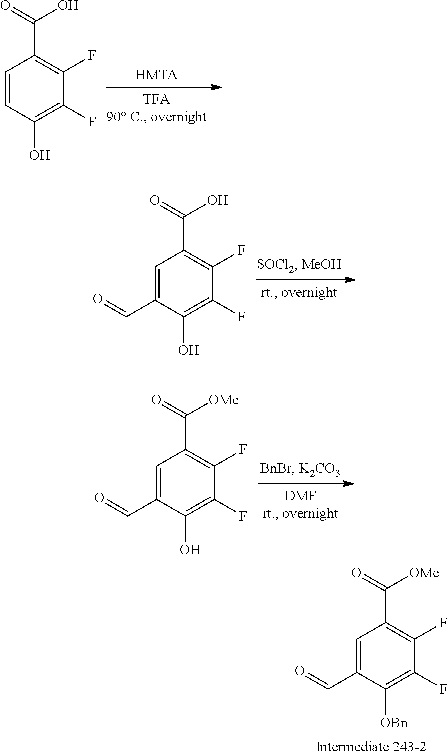

### Step 1: Synthesis of 2,3-difluoro-5-formyl-4-hydroxybenzoic acid

To a mixture of 2,3-difluoro-4-hydroxybenzoic acid (400 g, 2.30 mol mmol) in TFA (0.84 L) was added a solution of 1,3,5,7-tetraadamantanee (640.93 g, 4.572 mol) in TFA (1 L). The resulting mixture was stirred overnight at 90° C. The resulting mixture was concentrated under vacuum. The mixture was acidified to pH 2-4 with 4M aqueous HCl and stirred overnight. The mixture was filtered, rinsing with H₂O (500 mL). The filtrate was extracted with 3×1000 mL of ethyl acetate. The organic layers were combined, washed with sodium carbonate (aq.) and brine, dried and concentrated under vacuum. The residue was purified by silica gel column chromatography eluting with ethyl acetate/hexane (1/20) to give crude product. The crude product was re-crystallized from H₂O (100 mL) to afford 2,3-difluoro-5-formyl-4-hydroxybenzoic acid (310 g, 67%) as a yellow solid. MS (ESI) calcd. for C₈H₄F₂O₄, 202.01 m/z, found 201.05 [M−H]⁻.

### Step 2: Synthesis of methyl 2,3-difluoro-5-formyl-4-hydroxybenzoate

To a stirred solution of 2,3-difluoro-5-formyl-4-hydroxybenzoic acid (310 g, 1.534 mol) in MeOH (3 L) was added thionyl chloride (912.30 g, 7.669 mol) dropwise. The resulting mixture was stirred overnight at room temperature. The resulting mixture was concentrated under vacuum and the residue was purified by silica gel column chromatography using a gradient of ethyl acetate in dichloromethane/petroleum ether. The crude product was re-crystallized from petroleum ether (500 mL) to afford methyl 2,3-difluoro-5-formyl-4-hydroxybenzoate (162 g, 49%) as a yellow solid. MS (ESI) calcd. for C₉H₆F₂O₄, 216.02 m/z, found 217.05 [M+H]⁺.

### Step 3: Synthesis of methyl 4-(benzyloxy)-2,3-difluoro-5-formylbenzoate (Intermediate 243-2)

To a solution of methyl 2,3-difluoro-5-formyl-4-hydroxybenzoate (162 g, 750 mmol) in DMF (1.6 L) was added K₂CO₃ (310.76 g, 2248.5 mmol) and BnBr (384.58 g, 2248.5 mmol). The resulting mixture was stirred at room temperature overnight. The reaction was quenched with water (1.5 L). The resulting mixture was extracted with ethyl acetate (3 L×3). The combined organic layers were washed with water (2 L×3) and dried over anhydrous Na₂SO₄. The resulting mixture was concentrated under vacuum. The residue was purified by silica gel column chromatography, eluting with a gradient of ethyl acetate in dichloromethane/petroleum ether. The crude product was re-crystallized from petroleum ether (200 mL) to afford methyl 4-(benzyloxy)-2,3-difluoro-5-formylbenzoate (Intermediate 243-2) (182 g, 79%) as a yellow solid. ¹H NMR (400 MHz, DMSO-d₆) δ (ppm): 10.07 (s, 1H), 8.03-8.05 (m, 1H), 7.24-7.46 (m, 6H), 5.47 (s, 2H), 3.88 (s, 3H).

### Example 244: N-{3-[(1S)-5-[2-(2-aminopyridin-3-yl)-5-(pyrazol-1-yl)imidazo[4,5-b]pyridin-3-yl]-2,3-dihydro-1H-inden-1-yl]-6-formyl-4-oxoquinazolin-7-yl}methanesulfonamide

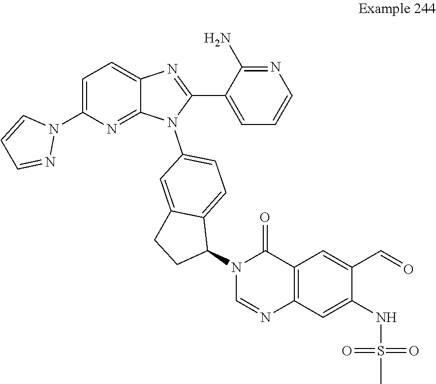 [SMILES: CS(=O)(=O)Nc1cc2ncn([C@H]3CCc4cc(-n5c(-c6cccnc6N)nc6ccc(-n7cccn7)nc65)ccc43)c(=O)c2cc1C=O]

Example 244 was prepared in a manner analogous to Example 220 (omitting Step 2) using Intermediate 33-1 in place of Intermediate 131-2, Intermediate 244-1 in place of Intermediate 220-1 and a reaction time of 60 h for the final step. MS (ESI) calcd. for C₃₃H₂₆N₁₀O₄S, 658.19, m/z, found 659.25 [M+H]⁺. ¹H NMR (400 MHz, DMSO-d₆) δ (ppm): 10.75 (s, 1H), 10.17 (s, 1H), 8.79 (s, 1H), 8.40-8.51 (m, 2H), 8.25-8.34 (m, 1H), 8.11-7.16 (m, 1H), 8.02-8.07 (m, 1H), 7.85-7.87 (m, 2H), 7.68-7.70 (m, 1H), 7.60-7.63 (m, 1H), 7.48-7.55 (m, 3H), 6.89-6.92 (m, 2H), 6.56-6.59 (m, 1H), 6.35-6.39 (m, 1H), 3.26-3.33 (m, 4H), 3.06-3.10 (m, 1H), 2.75-2.77 (m, 1H), 2.38-2.41 (m, 1H).

### Intermediate 244-1: 2-amino-5-formyl-4-(methylsulfonamido)benzoic acid

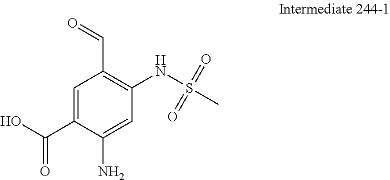 [SMILES: CS(=O)(=O)Nc1cc(N)c(C(=O)O)cc1C=O]

### Synthetic Route:

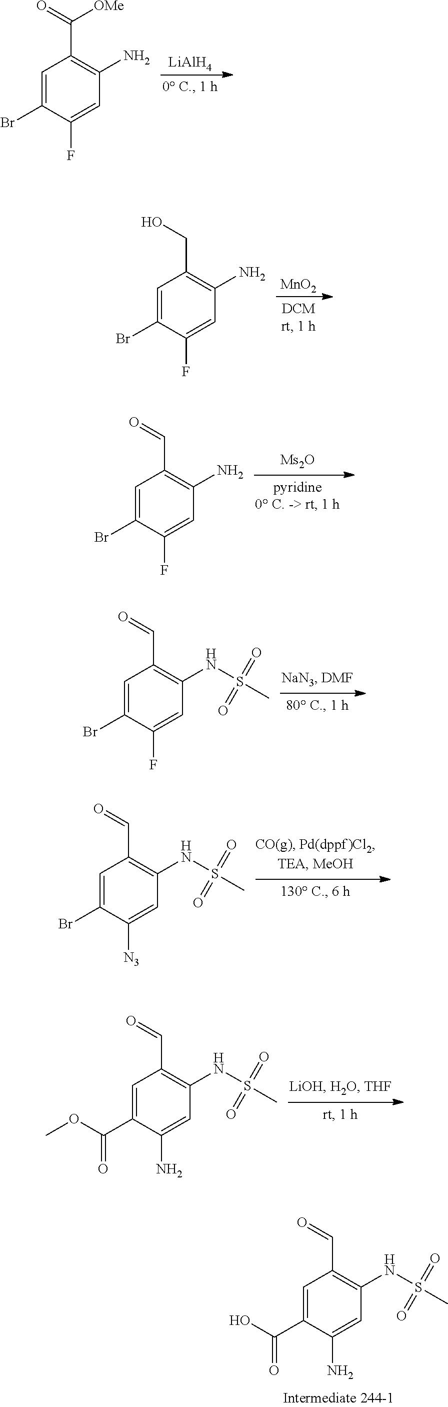

### Step 1: Synthesis of (2-amino-5-bromo-4-fluorophenyl) methanol

To a solution of methyl 2-amino-5-bromo-4-fluorobenzoate (3.00 g, 12.1 mmol) in THF (30 mL) was added LiAlH₄ (1.15 g, 30.2 mmol) at 0° C. After being stirred for 1 h at 0° C., the reaction was quenched with water (50 mL) at 0° C. and the mixture was extracted with ethyl acetate (3×150 mL). The combined organic extracts were washed with brine (3×100 mL), dried over anhydrous Na₂SO₄, filtered and concentrated under vacuum to yield (2-amino-5-bromo-4-fluorophenyl)methanol (2.5 g, 94% yield) as a light yellow solid. MS (ESI) calcd. for C₇H₇BrFNO, 218.97 m/z, found 219.15 [M+H]⁺.

### Step 2: Synthesis of 2-amino-5-bromo-4-fluorobenzaldehyde

To a solution of (2-amino-5-bromo-4-fluorophenyl)methanol (2.5 g, 11 mmol) in DCM (30 mL) was added MnO₂ (9.88 g, 114 mmol) at 0° C. After being stirred for 1 h at room temperature, the resulting mixture was filtered and the filter cake was washed with DCM (3×50 mL). The filtrate was concentrated under reduced pressure giving 2-amino-5-bromo-4-fluorobenzaldehyde (2.00 g, 81% yield) as a yellow solid. MS (ESI) calcd. for C₇H₅BrFNO, 216.95 m/z, found 217.15 [M+H]⁺.

### Step 3: Synthesis of N-(4-bromo-5-fluoro-2-formylphenyl)methanesulfonamide

To a solution of 2-amino-5-bromo-4-fluorobenzaldehyde (2.1 g, 9.6 mmol) in pyridine (20 mL) was added methanesulfonic anhydride (8.39 g, 48.2 mmol) at 0° C. The reaction was stirred for 1 h at room temperature. The reaction was quenched by the addition of water (20 mL) at 0° C. The aqueous layer was extracted with ethyl acetate (3×100 mL). The combined organic layers were washed with water (3×100 mL) and dried over anhydrous NaSO₄. After filtration, the filtrate was concentrated under reduced pressure. The residue was purified by silica gel column chromatography using a gradient of ethyl acetate in petroleum ether to afford N-(4-bromo-5-fluoro-2-formylphenyl)methanesulfonamide (2.5 g, 88% yield) as a light yellow solid. MS (ESI) calcd. for C₈H₇BrFNO₃S, 294.93 m/z, found 295.15 [M+H]⁺.

### Step 4: Synthesis of N-(5-azido-4-bromo-2-formylphenyl)methanesulfonamide

A mixture of N-(4-bromo-5-fluoro-2-formylphenyl)methanesulfonamide (200 mg, 0.675 mmol) and NaN₃ (65.9 mg, 1.01 mmol) in DMF (5 mL) was stirred at 80° C. for 1 h. The mixture was purified directly by reverse-phase flash column chromatography on C18 silica gel using a gradient of acetonitrile in water (+0.05% NH₄HCO₃) to afford N-(5-azido-4-bromo-2-formylphenyl)methanesulfonamide (150 mg, 70% yield) as a light yellow solid. MS (ESI) calcd. for C₈H₇BrN₄O₃S, 317.94 m/z, found 318.95 [M+H]⁺.

### Step 5: Synthesis of methyl 2-amino-5-formyl-4-methanesulfonamidobenzoate

To a solution of N-(5-azido-4-bromo-2-formylphenyl)methanesulfonamide (150 mg, 0.470 mmol) in MeOH (5 mL) was added Pd(dppf)C₁₂ (34 mg, 0.047 mmol) and triethylamine (237.8 mg, 2.350 mmol) in a pressure tank. The mixture was purged with nitrogen for 5 min and then was pressurized to 20 atm with carbon monoxide at 130° C. for 6 h. The reaction mixture was cooled to 0° C. and quenched by the addition of water (15 mL). The aqueous layer was extracted with ethyl acetate (3×50 mL). The combined organic layers were washed with water (3×50 mL) and dried over anhydrous NaSO₄. After filtration, the filtrate was concentrated under reduced pressure. The residue was purified by silica gel column chromatography, eluting with 0-40% ethyl acetate in petroleum ether to afford methyl 2-amino-5-formyl-4-methanesulfonamidobenzoate (80 mg, 63% yield) as a light yellow solid. MS (ESI) calcd. for C₁₀H₁₂N₂O₅S, 272.05 m/z, found 273.10 [M+H]⁺.

### Step 6: Synthesis of 2-amino-5-formyl-4-methanesulfonamidobenzoic acid (Intermediate 244-1)

To a solution of methyl 2-amino-5-formyl-4-methanesulfonamidobenzoate (80 mg, 0.29 mmol) in THF (4 mL) was added a solution of LiOH (35.2 mg, 1.47 mmol) in water (1 mL). The obtained solution was stirred at room temperature for 1 h. The resulting mixture was partially concentrated under reduced pressure then acidified to pH=6 with HCl (2 M). The solid was collected by filtration to afford 2-amino-5-formyl-4-methanesulfonamidobenzoic acid (Intermediate 244-1) (46 mg, 61% yield) as a light brown solid. MS (ESI) calcd. for C₉H₁₀N₂O₅S, 258.03 m/z, found 259.05 [M+H]⁺.

### Example 245: N-[(1S)-5-[2-(2-aminopyridin-3-yl)-7-(difluoromethyl)-5-(pyrazol-1-yl)imidazo[4,5-b]pyridin-3-yl]-2,3-dihydro-1H-inden-1-yl]-3-formyl-4-hydroxybenzamide

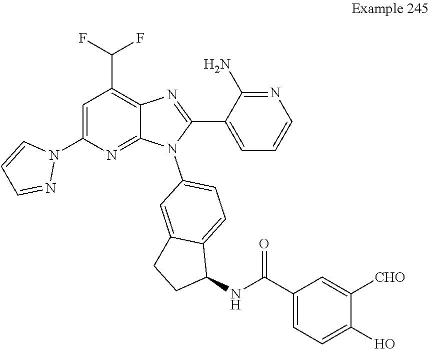 [SMILES: Nc1ncccc1-c1nc2c(C(F)F)cc(-n3cccn3)nc2n1-c1ccc2c(c1)CC[C@@H]2NC(=O)c1ccc(O)c(C=O)c1]

### Synthetic Route:

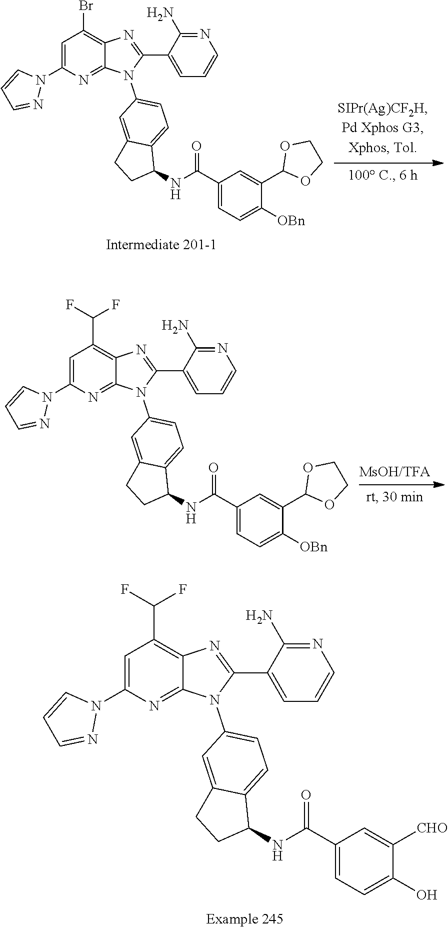 [SMILES: Nc1ncccc1-c1nc2c(Br)cc(-n3cccn3)nc2n1-c1ccc2c(c1)CC[C@@H]2NC(=O)c1ccc(OCc2ccccc2)c(C2OCCO2)c1 ; Nc1ncccc1-c1nc2c(C(F)F)cc(-n3cccn3)nc2n1-c1ccc2c(c1)CC[C@@H]2NC(=O)c1ccc(OCc2ccccc2)c(C2OCCO2)c1 ; Nc1ncccc1-c1nc2c(C(F)F)cc(-n3cccn3)nc2n1-c1ccc2c(c1)CC[C@@H]2NC(=O)c1ccc(O)c(C=O)c1]

### Step 1: Synthesis of N-[(1S)-5-[2-(2-aminopyridin-3-yl)-7-(difluoromethyl)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide

To a mixture of N-[(1S)-5-[2-(2-aminopyridin-3-yl)-7-bromo-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (Intermediate 201-1) (100 mg, 0.130 mmol) in toluene (5 mL) was added XPhos (12 mg, 0.026 mmol), XPhos Pd G3 (22 mg, 0.026 mmol) and {1,3-bis[2,6-bis(propan-2-yl)phenyl]imidazolidin-2-ylidene}(difluoromethyl)silver (143 mg, 0.260 mmol) in the dark. The mixture was stirred for 6 h at 100° C. under a nitrogen atmosphere in the dark. The reaction was quenched with water (50 mL) at room temperature and the mixture was extracted with ethyl acetate (3×50 mL). The combined organic extracts were washed with brine (3×10 mL), dried over anhydrous Na₂SO₄, filtered and concentrated. The residue was purified by reverse-phase flash column chromatography on C18 silica gel using a gradient of acetonitrile in water (+0.05% TFA) to afford N-[(1S)-5-[2-(2-aminopyridin-3-yl)-7-(difluoromethyl)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (30 mg, 31% yield) as a light yellow solid. MS (ESI) calcd. for C₄₁H₃₄F₂N₈O₄, 740.27 m/z, found 741.30 [M+H]⁺.

### Step 2: Synthesis of N-[(1S)-5-[2-(2-aminopyridin-3-yl)-7-(difluoromethyl)-5-(pyrazol-1-yl)imidazo[4,5-b]pyridin-3-yl]-2,3-dihydro-1H-inden-1-yl]-3-formyl-4-hydroxybenzamide (Example 245)

To a cooled (0° C.) solution of N-[(1S)-5-[2-(2-aminopyridin-3-yl)-7-(difluoromethyl)-5-(pyrazol-1-yl)imidazo[4,5-b]pyridin-3-yl]-2,3-dihydro-1H-inden-1-yl]-4-(benzyloxy)-3-(1,3-dioxolan-2-yl)benzamide (30 mg, 0.040 mmol) in TFA (1 mL) was added MsOH (0.2 mL) and the reaction mixture was stirred at room temperature for 30 min. The mixture was purified by Prep-HPLC on a XSelect CSH Prep C18 OBD Column using a gradient of acetonitrile in water (+0.1% formic acid) to afford N-[(1S)-5-[2-(2-aminopyridin-3-yl)-7-(difluoromethyl)-5-(pyrazol-1-yl)imidazo[4,5-b]pyridin-3-yl]-2,3-dihydro-1H-inden-1-yl]-3-formyl-4-hydroxybenzamide (Example 245) (4.0 mg, 16% yield) as a light yellow solid. MS (ESI) calcd. for C₃₂H₂₄F₂N₈O₃, 606.19, m/z, found 607.25 [M+H]⁺. ¹H NMR (400 MHz, DMSO-d₆) δ (ppm): 10.14 (s, 1H), 8.32-8.34 (m, 1H), 8.19-8.21 (m, 1H), 7.93-8.06 (m, 3H), 7.71-7.80 (m, 1H), 7.37-7.69 (m, 1H), 7.24-7.32 (m, 3H), 6.94-7.21 (m, 2H), 6.46-6.57 (m, 2H), 5.49-5.56 (m, 1H), 2.97-2.99 (m, 1H), 2.85-2.87 (m, 1H), 2.49-2.51 (m, 1H), 1.92-2.17 (m, 1H). ¹⁹F NMR (376 MHz, DMSO-d₆) δ (ppm): −115.4.

### Example 246: N-[(1S)-5-[2-(2-aminopyridin-3-yl)-5-{4H,6H,7H-pyrazolo[3,2-c][1,4]oxazin-2-yl}imidazo[4,5-b]pyridin-3-yl]-2,3-dihydro-1H-inden-1-yl]-2-fluoro-5-formyl-4-hydroxybenzamide

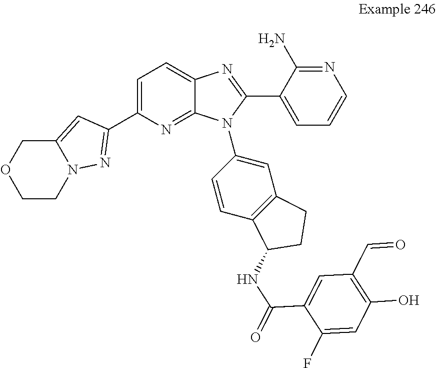 [SMILES: Nc1ncccc1-c1nc2ccc(-c3cc4n(n3)CCOC4)nc2n1-c1ccc2c(c1)CC[C@@H]2NC(=O)c1cc(C=O)c(O)cc1F]

Example 246 was prepared in a manner analogous to Example 193 using Intermediate 246-1 in place of Intermediate 193-1 and 2-bromo-6,7-dihydro-4H-pyrazolo[5,1-c][1,4]oxazine in place of 2-bromo-5-fluoro-1,3-thiazole. MS (ESI) calcd. for C₃₄H₂₇FN₈O₄: 630.21 m/z, found: 631.30 [M+H]⁺. ¹H NMR (400 MHz, DMSO-d₆) δ (ppm): 10.15 (s, 1H), 8.18-8.25 (m, 1H), 7.90-8.08 (m, 3H), 7.31-7.45 (m, 2H), 7.21-7.30 (m, 2H), 6.41-6.59 (m, 3H), 5.50-5.60 (m, 1H), 4.75-4.90 (m, 2H), 4.05-4.25 (m, 4H), 2.95-3.05 (m, 1H), 2.80-2.94 (m, 1H), 2.33-2.45 (m, 1H), 1.95-2.11 (m, 1H). ¹⁹F NMR (400 MHz, DMSO-d₆) δ (ppm): −103.47.

### Intermediate 246-1: N-[(1S)-5-[2-(2-aminopyridin-3-yl)-5-bromoimidazo[4,5-b]pyridin-3-yl]-2,3-dihydro-1H-inden-1-yl]-4-(benzyloxy)-5-(1,3-dioxolan-2-yl)-2-fluorobenzamide

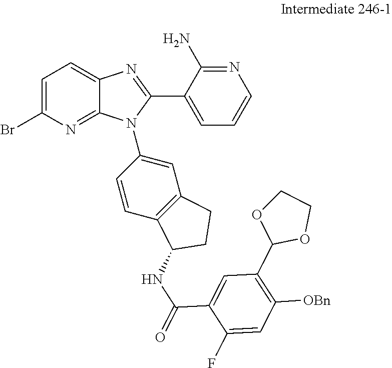 [SMILES: Nc1ncccc1-c1nc2ccc(Br)nc2n1-c1ccc2c(c1)CC[C@@H]2NC(=O)c1cc(C2OCCO2)c(OCc2ccccc2)cc1F]

Intermediate 246-1 was prepared in a manner analogous to Intermediate 193-1 using Intermediate 16-1 in place of Intermediate 118-1. MS (ESI) calcd. for C₃₇H₃₀BrFN₆O₄: 720.15 m/z, found: 721.10 [M+H]⁺.

### Example 247: N-[(1S)-5-[2-(2-aminopyridin-3-yl)-5-{4H,5H,6H-pyrrolo[1,2-b]pyrazol-2-yl}imidazo[4,5-b]pyridin-3-yl]-2,3-dihydro-1H-inden-1-yl]-2-fluoro-5-formyl-4-hydroxybenzamide

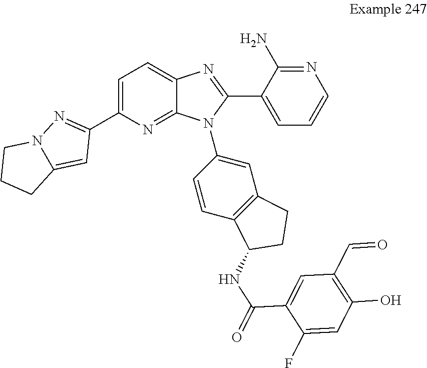 [SMILES: Nc1ncccc1-c1nc2ccc(-c3cc4n(n3)CCC4)nc2n1-c1ccc2c(c1)CC[C@@H]2NC(=O)c1cc(C=O)c(O)cc1F]

Example 247 was prepared in a manner analogous to Example 193 using Intermediate 246-1 in place of Intermediate 193-1 and 2-bromo-5,6-dihydro-4H-pyrrolo[1,2-b]pyrazole in place of 2-bromo-5-fluoro-1,3-thiazole. MS (ESI) calcd. for C₃₄H₂₇FN₈O₃: 614.22 m/z, found: 615.30 [M+H]⁺. ¹H NMR (400 MHz, DMSO-d₆) δ (ppm): 10.21 (s, 1H), 8.20-8.31 (m, 1H), 7.95-8.15 (m, 3H), 7.75-7.85 (m, 1H), 7.38-7.50 (m, 2H), 7.29-7.37 (m, 1H), 6.75-6.91 (m, 2H), 6.38-6.45 (m, 1H), 5.49-5.65 (m, 1H), 4.08-4.20 (m, 2H), 2.75-3.10 (m, 4H), 2.30-2.70 (m, 3H), 1.99-2.15 (m, 1H). ¹⁹F NMR (400 MHz, DMSO-d₆) δ (ppm): −101.74. (TFA salt).

### Example 248: (S)—N-(5-(2-(2-aminopyridin-3-yl)-6-fluoro-5-(1H-pyrazol-1-yl)-3H-imidazo[4,5-b]pyridin-3-yl)-2,3-dihydro-1H-inden-1-yl)-3-formyl-4-hydroxybenzamide

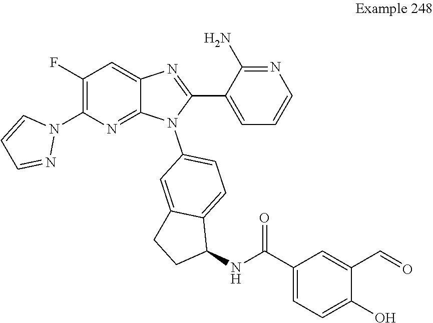 [SMILES: Nc1ncccc1-c1nc2cc(F)c(-n3cccn3)nc2n1-c1ccc2c(c1)CC[C@@H]2NC(=O)c1ccc(O)c(C=O)c1]

Example 248 was prepared in a manner analogous to Example 7 using Intermediate 248-2 in place of Intermediate 1-2 and Intermediate 57-2 in place of Intermediate 5-1. MS (ESI) calcd. for C₃₁H₂₃FN₈O₃, 574.19 m/z, found 575.15 [M+H]⁺. ¹H NMR (400 MHz, DMSO-d₆) δ (ppm): 10.26 (s, 1H), 8.50-8.53 (m, 1H), 8.23-8.28 (m, 2H), 8.08-8.09 (m, 2H), 8.06-8.07 (m, 2H), 7.82-7.83 (m, 1H), 7.34-7.44 (m, 2H), 7.07-7.10 (m, 1H), 6.81-6.84 (m, 1H), 6.50-6.70 (m, 1H), 5.58-5.62 (m, 1H), 3.00-3.06 (m, 1H), 2.87-2.93 (m, 1H), 2.50-2.51 (m, 1H), 2.05-2.10 (m, 1H). ¹⁹F NMR (376 MHz, DMSO-d₆) δ (ppm): −133.55. (TFA salt).

### Intermediate 248-2: (S)-3-(3-(1-amino-2,3-dihydro-1H-inden-5-yl)-6-fluoro-5-(1H-pyrazol-1-yl)-3H-imidazo[4,5-b]pyridin-2-yl)pyridin-2-amine

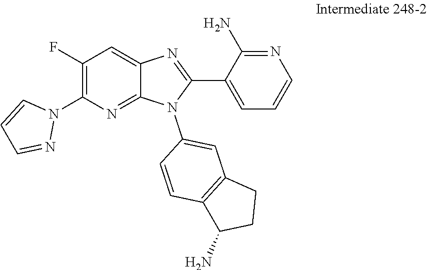 [SMILES: Nc1ncccc1-c1nc2cc(F)c(-n3cccn3)nc2n1-c1ccc2c(c1)CC[C@@H]2N]

Intermediate 248-2 was prepared in a manner analogous to Intermediate 69-1 (starting from Step 2) using Intermediate 248-1 in place of the nitro aniline and a reaction time of overnight for the condensation with the aldehyde. MS (ESI) calcd. for C₂₃H₁₉FN₈, 426.16 m/z, found 427.05 [M+H]⁺.

### Intermediate 248-1: (S)—N-(5-((5-fluoro-3-nitro-6-(1H-pyrazol-1-yl)pyridin-2-yl)amino)-2,3-dihydro-1H-inden-1-yl)acetamide

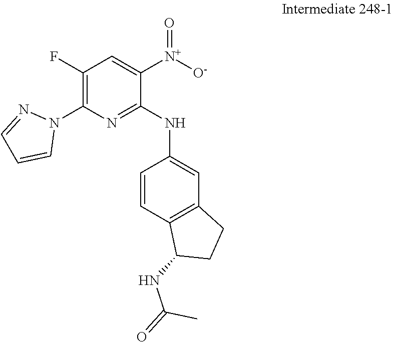 [SMILES: CC(=O)N[C@H]1CCc2cc(Nc3nc(-n4cccn4)c(F)cc3[N+](=O)[O-])ccc21]

Intermediate 248-1 was prepared in a manner analogous to Intermediate 163-1 using Intermediate 248-0 in place of 2,6-dichloro-3-nitropyridine, potassium carbonate in place of triethylamine, N,N-dimethylformamide in place of ethanol and room temperature for Step 3 instead of 60° C. MS (ESI) calcd. for C₁₉H₁₇FN₆O₃, 396.13 m/z, found 397.05 [M+H]⁺.

### Intermediate 248-0: 2-chloro-5-fluoro-3-nitro-6-(1H-pyrazol-1-yl)pyridine

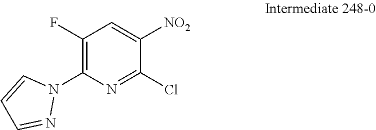 [SMILES: O=[N+]([O-])c1cc(F)c(-n2cccn2)nc1Cl]

### Synthetic Route:

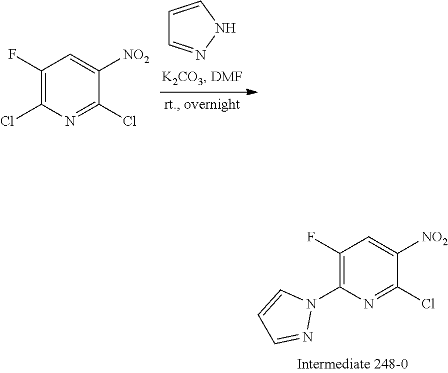

### Step 1: Synthesis of 2-chloro-5-fluoro-3-nitro-6-(1H-pyrazol-1-yl)pyridine (Intermediate 248-0)

To a stirred solution of 2,6-dichloro-3-fluoro-5-nitropyridine (1.0 g, 4.7 mmol) in DMF (30 mL) were added 1H-pyrazole (322.3 mg, 4.739 mol) and K₂CO₃ (1.308 g, 9.478 mmol). The resulting mixture was stirred at room temperature overnight. The reaction was quenched with saturated aqueous ammonium chloride (50 mL) at 0° C. then extracted with ethyl acetate (3×50 mL). The combined organic layers were concentrated under reduced pressure. The obtained residue was purified by silica gel column chromatography eluting with ethyl acetate/petroleum ether (0˜15%) to give 2-chloro-5-fluoro-3-nitro-6-(1H-pyrazol-1-yl)pyridine (Intermediate 248-0) (640 mg, 56% yield) as a yellow solid. MS (ESI) calcd. for C₈H₄ClFN₄O₂, 242.00 m/z, found 243.00 [M+H]⁺.

### Synthesis of Example 249: (S)-3-(5-(2-(2-aminopyridin-3-yl)-5-(1H-pyrazol-1-yl)-3H-imidazo[4,5-b]pyridin-3-yl)-2,3-dihydro-1H-inden-1-yl)-7-hydroxy-2-methyl-4-oxo-3,4-dihydroquinazoline-6-carbaldehyde

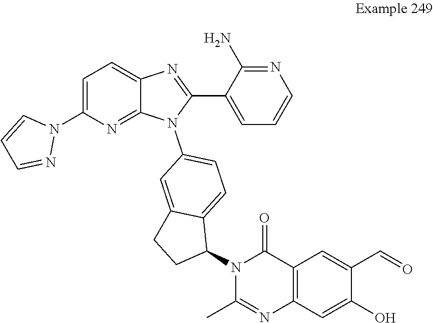 [SMILES: Cc1nc2cc(O)c(C=O)cc2c(=O)n1[C@H]1CCc2cc(-n3c(-c4cccnc4N)nc4ccc(-n5cccn5)nc43)ccc21]

### Synthetic Route:

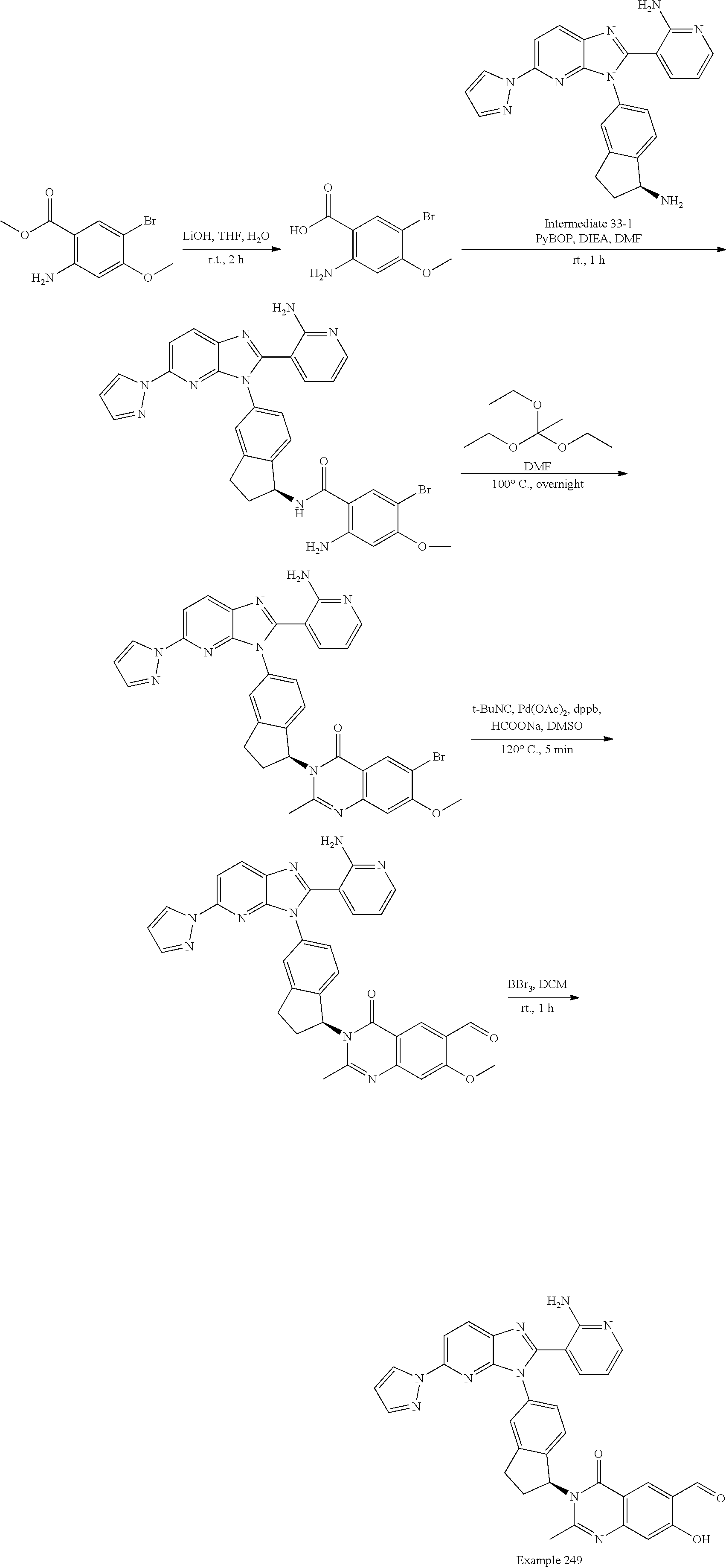

### Step 1: Synthesis of 2-amino-5-bromo-4-methoxybenzoic acid

A mixture of methyl 2-amino-5-bromo-4-methoxybenzoate (2.00 g, 7.69 mmol) and LiOH (23.1 mmol) in THF (20 mL) and H₂O (5 mL) was stirred at room temperature for 2 h. The resulting mixture was concentrated under reduced pressure. The mixture was adjusted to pH 7 with 1M aqueous HCl. The solid was collected by filtration to afford 2-amino-5-bromo-4-methoxybenzoic acid (1.5 g, 79% yield) as a yellow solid. MS (ESI) calcd. for C₈H₈BrNO₃: 244.97 m/z, found 246.10 [M+H]⁺.

### Step 2: Synthesis of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-5-bromo-4-methoxybenzamide

A solution of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (2.99 g, 7.32 mmol), 2-amino-5-bromo-4-methoxybenzoic acid (1.5 g, 6.096 mmol), PyBOP (3.49 g, 6.71 mmol) and N,N-diisopropylethylamine (3.94 g, 30.5 mmol) in DMF (50 mL) was stirred at room temperature for 1 h. The reaction was quenched with saturated aqueous ammonium chloride (30 mL) and extracted with ethyl acetate (30 mL×3). The combined organic layers were washed with brine and dried over anhydrous Na₂SO₄. After filtration, the filtrate was concentrated under reduced pressure to afford 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-5-bromo-4-methoxybenzamide (3 g, 71% yield) as a green solid. MS (ESI) calcd. for C₃₁H₂₆BrN₉O₂: 635.14 m/z, found 636.35 [M+H]⁺.

### Step 3: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-6-bromo-7-methoxy-2-methylquinazolin-4-one

To a solution of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-5-bromo-4-methoxybenzamide (1.0 g, 1.6 mmol) in DMF (20 mL) was added triethyl orthoacetate (1.27 g, 7.86 mmol) and the resulting mixture was stirred at 100° C. overnight. The resulting mixture was purified by reverse phase flash column chromatography on C18 silica gel using a gradient of acetonitrile in water (+0.05% NH₄HCO₃) to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-6-bromo-7-methoxy-2-methylquinazolin-4-one (300 mg, 29% yield) as a white solid. MS (ESI) calcd. for C₃₃H₂₆BrN₉O₂: 659.14 m/z, found 660.35 [M+H]⁺.

### Step 4: Synthesis of (S)-3-(5-(2-(2-aminopyridin-3-yl)-5-(1H-pyrazol-1-yl)-3H-imidazo[4,5-b]pyridin-3-yl)-2,3-dihydro-1H-inden-1-yl)-7-methoxy-2-methyl-4-oxo-3,4-dihydroquinazoline-6-carbaldehyde

A solution of 3-[(1S)-5-[2-(2-aminopyridin-3-yl)-5-(pyrazol-1-yl)imidazo[4,5-b]pyridin-3-yl]-2,3-dihydro-1H-inden-1-yl]-6-bromo-7-methoxy-2-methylquinazolin-4-one (300 mg, 0.454 mmol), tert-butyl isocyanide (94.4 mg, 1.14 mmol), sodium formate (92.7 mg, 1.36 mmol) and dppb (387.4 mg, 0.908 mmol) in DMSO (5 mL) was stirred at 120° C. for 5 min under nitrogen atmosphere. The resulting mixture was diluted with water (50 mL) and extracted with ethyl acetate (50 mL×3). The combined extracts were concentrated under vacuum and the resulting oil was purified by Prep-HPLC on a XSelect CSH Fluoro Phenyl column using a gradient of acetonitrile in water (+0.1% formic acid) to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-methoxy-2-methyl-4-oxoquinazoline-6-carbaldehyde (67 mg, 24.35% yield) as a white solid. MS (ESI) calcd. for C₃₄H₂₇N₉O₃ 609.22 m/z, found 610.10 [M+H]⁺.

### Step 5: Synthesis of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-2-methyl-4-oxoquinazoline-6-carbaldehyde (Example 249)

A mixture of 3-[(1S)-5-[2-(2-aminopyridin-3-yl)-5-(pyrazol-1-yl)imidazo[4,5-b]pyridin-3-yl]-2,3-dihydro-1H-inden-1-yl]-7-methoxy-2-methyl-4-oxoquinazoline-6-carbaldehyde (40 mg, 0.066 mmol) and BBr₃ (0.33 mL, 0.330 mmol, 1M in DCM) in DCM (5 mL) was stirred at room temperature for 1 h. The resulting mixture was concentrated to dryness and the resulting oil was purified by Prep-HPLC on a XSelect CSH Fluoro Phenyl column using a gradient of acetonitrile in water (+0.1% formic acid) to afford 3-[(1S)-5-[2-(2-aminopyridin-3-yl)-5-(pyrazol-1-yl)imidazo[4,5-b]pyridin-3-yl]-2,3-dihydro-1H-inden-1-yl]-7-hydroxy-2-methyl-4-oxoquinazoline-6-carbaldehyde (Example 249) (1.0 mg, 2% yield) as a white solid. MS (ESI) calcd. for C₃₃H₂₅N₉O₃, 595.21 m/z, found 596.30 [M+H]⁺. ¹H NMR (400 MHz, DMSO-d₆) δ (ppm): 10.02-10.31 (m, 1H), 8.15-8.46 (m, 3H), 7.84-8.15 (m, 2H), 7.71-7.81 (m, 1H), 7.32-7.49 (m, 1H), 7.16-7.29 (m, 2H), 7.04-7.16 (m, 1H), 6.85-7.00 (m, 1H), 6.49-6.59 (m, 1H), 6.39-6.49 (m, 1H), 5.95-6.05 (m, 1H), 3.42-3.48 (m, 1H), 3.16-3.28 (m, 1H), 2.91-3.09 (m, 1H), 2.69-2.83 (m, 3H), 1.15-1.26 (m, 1H).

### Example 250: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-cyano-5-formyl-4-hydroxybenzamide

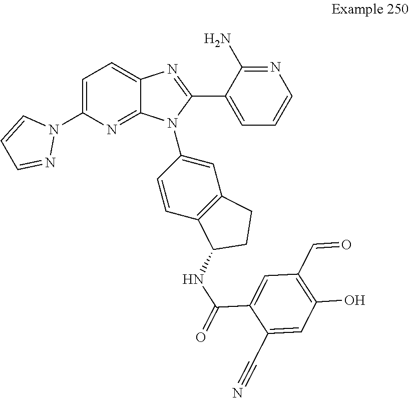 [SMILES: N#Cc1cc(O)c(C=O)cc1C(=O)N[C@H]1CCc2cc(-n3c(-c4cccnc4N)nc4ccc(-n5cccn5)nc43)ccc21]

### Synthetic Route:

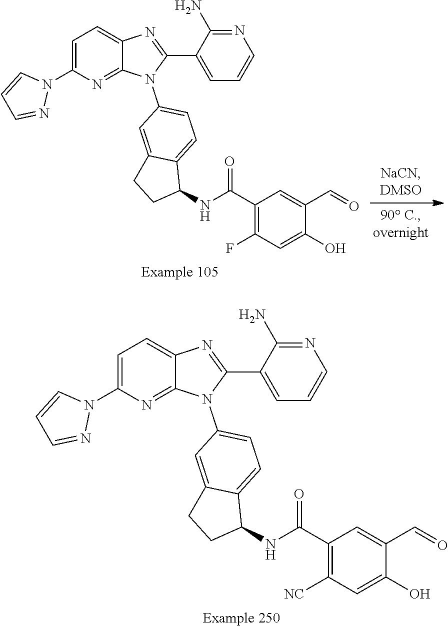 [SMILES: Nc1ncccc1-c1nc2ccc(-n3cccn3)nc2n1-c1ccc2c(c1)CC[C@@H]2NC(=O)c1cc(C=O)c(O)cc1F ; N#Cc1cc(O)c(C=O)cc1C(=O)N[C@H]1CCc2cc(-n3c(-c4cccnc4N)nc4ccc(-n5cccn5)nc43)ccc21]

### Step 1: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-cyano-5-formyl-4-hydroxybenzamide (Example 250)

A solution of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-fluoro-5-formyl-4-hydroxybenzamide (Example 105) (100 mg, 0.174 mmol) in DMSO (2 mL) was treated with NaCN (42.7 mg, 0.870 mmol) at room temperature under nitrogen atmosphere. The mixture was stirred overnight at 90° C. The reaction was quenched with H₂O (50 mL) at room temperature. The resulting mixture was extracted with ethyl acetate (3×50 mL). The combined organic layers were washed with brine (50 mL) and dried over anhydrous Na₂SO₄. After filtration, the filtrate was concentrated under reduced pressure. The crude product was purified by Prep-HPLC on a XBridge Prep OBD C18 Column using a gradient of acetonitrile in water (+10 mmol/L NH₄HCO₃) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-cyano-5-formyl-4-hydroxybenzamide (Example 250) (3.0 mg, 3% yield) as a yellow solid. MS (ESI) calcd. for C₃₂H₂₃N₉O₃, 581.19 m/z, 582.10 [M+H]⁺. ¹H NMR (400 MHz, DMSO-d₆) δ (ppm): 8.23-8.44 (m, 2H), 7.63-8.09 (m, 4H), 7.29-7.45 (m, 1H), 6.97-7.25 (m, 3H), 6.83-6.97 (m, 1H), 6.50-6.59 (m, 1H), 6.39-6.49 (m, 1H), 5.89-6.03 (m, 1H), 2.79-3.03 (m, 2H), 2.61-2.65 (m, 1H), 2.36-2.41 (m, 1H). (1 signal missing due to overlap with water).

### Example 251: N-(5-(2-(2-aminopyridin-3-yl)-5-(1H-pyrazol-1-yl)-3H-imidazo[4,5-b]pyridin-3-yl)-2-hydroxy-2,3-dihydro-1H-inden-1-yl)-3-formyl-4-hydroxybenzamide

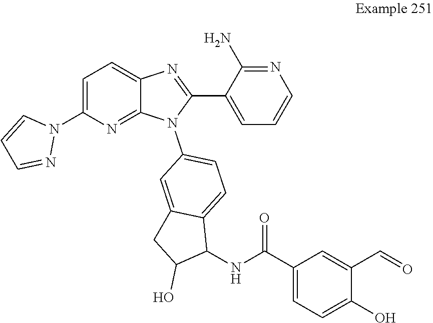 [SMILES: Nc1ncccc1-c1nc2ccc(-n3cccn3)nc2n1-c1ccc2c(c1)CC(O)C2NC(=O)c1ccc(O)c(C=O)c1]

Example 251 was prepared in a manner analogous to Example 5 using Intermediate 251-2 in place of Intermediate 1-3. MS (ESI) calcd. for C₃₁H₂₄N₈O₄, 572.19 m/z, found: 573.10 [M+H]⁺. ¹H NMR (400 MHz, DMSO-d₆) δ (ppm): 10.33 (s, 1H), 8.92-8.94 (m, 1H), 8.34-8.37 (m, 3H), 8.11-8.13 (m, 1H), 8.01-8.02 (m, 1H), 7.94-7.96 (m, 1H), 7.81-7.82 (m, 1H), 7.35 (s, 1H), 7.27-7.31 (m, 3H), 7.06-7.09 (m, 1H), 6.92-6.93 (m, 2H), 6.55-6.56 (m, 1H), 6.44-6.47 (m, 1H), 5.46-5.47 (m, 1H), 5.35-5.39 (m, 1H), 4.52-4.54 (m, 1H), 3.20-3.26 (m, 1H), 2.77-2.83 (m, 1H).

### Intermediate 251-2: 1-amino-5-(2-(2-aminopyridin-3-yl)-5-(1H-pyrazol-1-yl)-3H-imidazo[4,5-b]pyridin-3-yl)-2,3-dihydro-1H-inden-2-ol

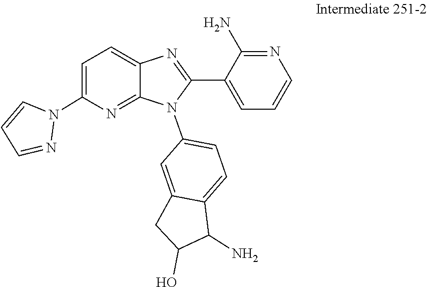 [SMILES: Nc1ncccc1-c1nc2ccc(-n3cccn3)nc2n1-c1ccc2c(c1)CC(O)C2N]

Intermediate 251-2 was prepared in a manner analogous to Intermediate 13-2 using Intermediate 251-1 in place of Intermediate 13-1 and 4N HCl in dioxane for 2 h at room temperature instead of conc HCl in methanol overnight at 90° C. MS (ESI) calcd. for C₂₃H₂₀N₈O, 424.18 m/z, found: 425.15 [M+H]⁺.

### Intermediate 251-1: tert-butyl (5-bromo-2-hydroxy-2,3-dihydro-1H-inden-1-yl)carbamate

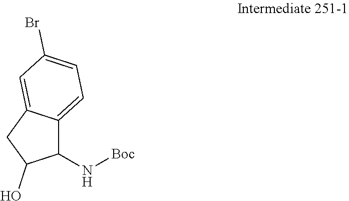 [SMILES: CC(C)(C)OC(=O)NC1c2ccc(Br)cc2CC1O]

### Synthetic Route:

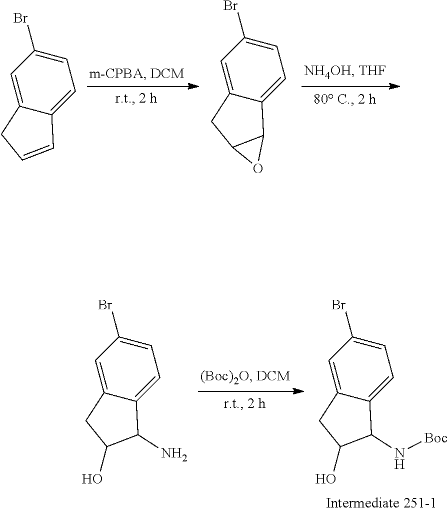 [SMILES: Brc1ccc2c(c1)CC=C2 ; Brc1ccc2c(c1)CC1OC21 ; NC1c2ccc(Br)cc2CC1O ; CC(C)(C)OC(=O)NC1c2ccc(Br)cc2CC1O]

### Step 1: Synthesis of 4-bromo-1a,6a-dihydro-6H-indeno[1,2-b]oxirene

To a solution of 5-bromo-3H-indene (2.0 g, 10 mmol) in DCM (50 mL) was added m-CPBA (3.54 g, 20.5 mmol) and the resulting mixture was stirred at room temperature for 2 h. The reaction mixture was quenched by addition of saturated aq. Na₂CO₃ (30 mL). The aqueous layer was extracted with ethyl acetate (3×30 mL). The combined organic phases were washed with brine (3×30 mL) and dried over anhydrous sodium sulfate. After filtration, the filtrate was concentrated under reduced pressure. The residue obtained was purified by silica gel column chromatography (0-10% ethyl acetate in petroleum ether) to afford 4-bromo-1aH,6H,6aH-indeno[1,2-b]oxirene (0.9 g, 41% yield) as a white solid. MS (ESI) calcd. for C₉H₇BrO, 209.97 m/z, found: 211.10 [M+H]⁺.

### Step 2: Synthesis of 1-amino-5-bromo-2,3-dihydro-1H-inden-2-ol

To a solution of 4-bromo-1aH,6H,6aH-indeno[1,2-b]oxirene (0.90 g, 4.3 mmol) in THF (15 mL) was added NH₄OH (15 mL, 390 mmol, 30%) at room temperature. The mixture was stirred at 80° C. for 2 h. After cooling to room temperature, the reaction was quenched with water (5 mL) and the mixture was extracted with ethyl acetate (3×15 mL). The combined organic extracts were washed with brine (3×10 mL) and dried over anhydrous Na₂SO₄. After filtration, the filtrate was concentrated under reduced pressure to afford 1-amino-5-bromo-2,3-dihydro-1H-inden-2-ol (1.0 g, crude) as a light yellow solid. MS (ESI) calcd. for C₉H₁₀BrNO, 226.99 m/z, found 228.10 [M+H]⁺.

### Step 3: Synthesis of tert-butyl (5-bromo-2-hydroxy-2,3-dihydro-1H-inden-1-yl)carbamate

To a solution of 1-amino-5-bromo-2,3-dihydro-1H-inden-2-ol (1.0 g, 4.4 mmol) in DCM (30 mL) was added (Boc)₂O (1.15 g, 5.26 mmol) and the resulting mixture was stirred at room temperature for 18 h. The reaction was quenched with water (10 mL), and the mixture was extracted with DCM (3×25 mL). The combined organic extracts were washed with brine (3×10 mL) and dried over anhydrous Na₂SO₄. After filtration, the filtrate was concentrated under reduced pressure. The crude product was purified by silica gel column chromatography (0-10% ethyl acetate in petroleum ether) to afford tert-butyl N-(5-bromo-2-hydroxy-2,3-dihydro-1H-inden-1-yl)carbamate (Intermediate 251-1) (500 mg, 35%) as a yellow solid. MS (ESI) calcd. for C₁₄H₁₈BrNO₃, 327.05 m/z, found: 328.10 [M+H]⁺.

### Example 252: N-(3-(2-(2-aminopyridin-3-yl)-5-(1H-pyrazol-1-yl)-3H-imidazo[4,5-b]pyridin-3-yl)-1,1a,6,6a-tetrahydrocyclopropa[a]inden-6-yl)-3-formyl-4-hydroxybenzamide

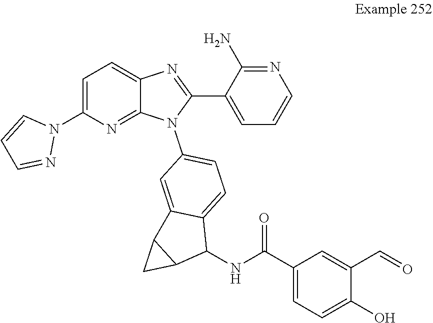 [SMILES: Nc1ncccc1-c1nc2ccc(-n3cccn3)nc2n1-c1ccc2c(c1)C1CC1C2NC(=O)c1ccc(O)c(C=O)c1]

Example 252 was prepared in a manner analogous to Example 12 using Intermediate 252-1 in place of Intermediate 1-3. MS (ESI) calcd. for C₃₂H₂₄N₈O₃: 568.20 m/z, found: 569.30 [M+H]⁺. ¹H-NMR (400 MHz, DMSO-d₆) δ (ppm): 10.26-10.28 (m, 1H), 8.25-8.43 (m, 3H), 7.99-8.08 (m, 3H), 7.82 (s, 1H), 7.68-7.71 (m, 1H), 7.28-7.52 (m, 3H), 7.06-7.10 (m, 1H), 6.81-6.82 (m, 1H), 6.56-6.58 (m, 1H), 5.87-5.89 (m, 0.5H), 5.39-5.41 (m, 0.5H), 2.59-2.61 (m, 0.5H), 2.50-2.51 (m, 0.5H), 2.15-2.16 (m, 0.5H), 2.04-2.06 (m, 0.5H), 1.29-1.31 (m, 0.5H), 0.98-0.99 (m, 0.5H), 0.73-0.74 (m, 0.5H), 0.20-0.21 (m, 0.5H). (TFA salt)

### Intermediate 252-1: 3-(3-(6-amino-1,1a,6,6a-tetrahydrocyclopropa[a]inden-3-yl)-5-(1H-pyrazol-1-yl)-3H-imidazo[4,5-b]pyridin-2-yl)pyridin-2-amine

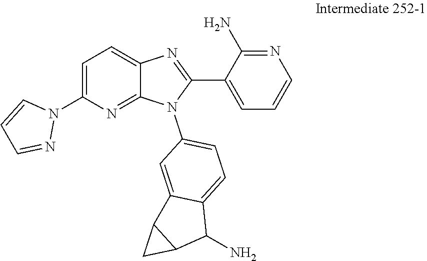 [SMILES: Nc1ncccc1-c1nc2ccc(-n3cccn3)nc2n1-c1ccc2c(c1)C1CC1C2N]

### Synthetic Route:

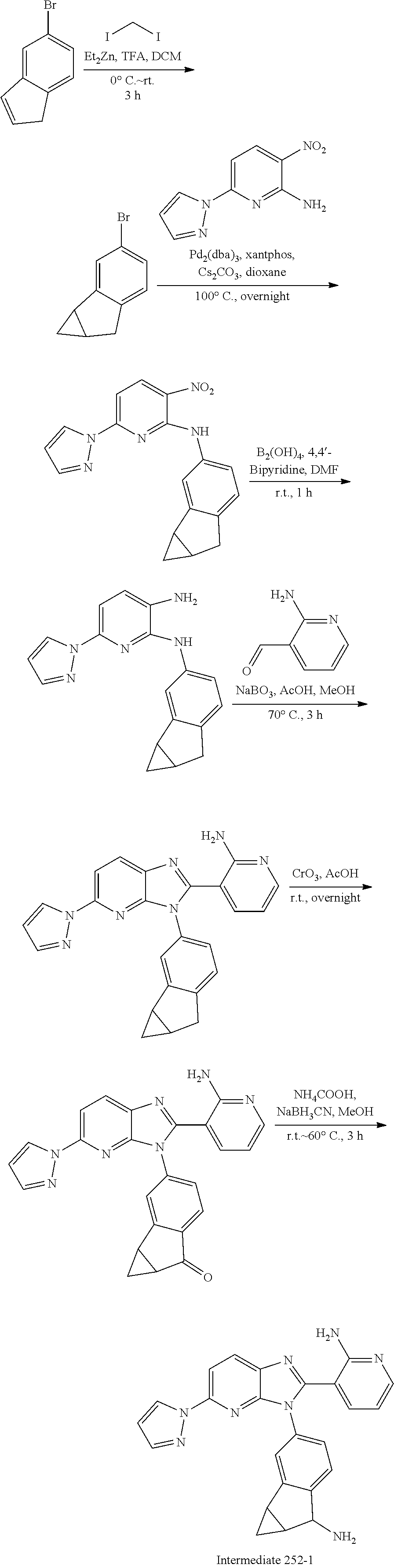

### Step 1: Synthesis of 3-bromo-1H,1aH,6H,6aH-cyclopropa[a]indene

To a solution of diethylzinc (20.5 mL, 20.5 mmol, 1 M in hexane) in DCM (20 mL) was added TFA (2.34 g, 20.5 mmol) in DCM (10 mL) at 0° C. After stirring for 20 min, a solution of diiodomethane (5.49 g, 20.5 mmol) in DCM (10 mL) was added. After stirring for 20 min, a solution of 5-bromo-1H-indene (2.00 g, 10.3 mmol) in DCM (10 mL) was added. The mixture was warmed to room temperature and stirred for 2 h. The reaction mixture was quenched with HCl (2N, aq.) (10 mL) and the resulting mixture was extracted with ethyl acetate (3×50 mL). The organic layers were combined, dried over Na₂SO₄, filtered and concentrated. The residue was purified by silica gel column chromatography (0˜100% ethyl acetate in petroleum ether) to afford 3-bromo-1H,1aH,6H,6aH-cyclopropa[a]indene (1.1 g, 51% yield) as a colorless oil. ¹H NMR (400 MHz, DMSO-d₆) δ (ppm): 7.46-7.47 (m, 1H), 7.21-7.26 (m, 1H), 7.08-7.10 (m, 1H), 3.03-3.09 (m, 1H), 2.81-2.86 (m, 1H), 2.36-2.41 (m, 1H), 1.88-1.93 (m, 1H), 1.05-1.12 (m, 1H), 0.20-0.21 (m, 1H).

### Step 2: Synthesis of N-{1H,1aH,6H,6aH-cyclopropa[a]inden-3-yl}-3-nitro-6-(pyrazol-1-yl)pyridin-2-amine

To a solution of 3-bromo-1H,1aH,6H,6aH-cyclopropa[a]indene (1.1 g, 5.3 mmol) in dioxane (20 mL) was added 3-nitro-6-(pyrazol-1-yl)pyridin-2-amine (1.08 g, 5.26 mmol), Pd₂(dba)₃ (481.8 mg, 0.526 mmol, 0.1 equiv), XantPhos (608.8 mg, 1.052 mmol) and Cs₂CO₃ (3.43 g, 10.5 mmol). The resulting mixture was maintained under nitrogen and stirred at 90° C. overnight. After cooling to room temperature, the reaction was quenched with H₂O (50 mL). The resulting mixture was extracted with ethyl acetate (3×50 mL). The organic layers were combined, dried over Na₂SO₄, filtered and concentrated. The residue obtained was purified by silica gel column chromatography using a gradient of ethyl acetate in petroleum ether to afford N-{1H,1aH,6H,6aH-cyclopropa[a]inden-3-yl}-3-nitro-6-(pyrazol-1-yl)pyridin-2-amine (1.6 g, 91% yield) as a yellow solid. MS (ESI) calcd. for C₁₈H₁₅N₅O₂: 333.12 m/z, found: 334.15 [M+H]⁺.

### Step 3: Synthesis of N2-{1H,1aH,6H,6aH-cyclopropa[a]inden-3-yl}-6-(pyrazol-1-yl)pyridine-2,3-diamine

To a solution of N-{1H,1aH,6H,6aH-cyclopropa[a]inden-3-yl}-3-nitro-6-(pyrazol-1-yl)pyridin-2-amine (1.4 g, 4.2 mmol) in DMF (20 mL) was added B₂(OH)₄ (1.13 g, 12.6 mmol) and 4-(pyridin-4-yl)pyridine (32.8 mg, 0.210 mmol). The resulting mixture was stirred at rt for 1 h then purified by reverse-phase flash column chromatography on C18 silica gel using a gradient of acetonitrile in water (+0.05% NH₄HCO₃) to afford N2-{1H,1aH,6H,6aH-cyclopropa[a]inden-3-yl}-6-(pyrazol-1-yl)pyridine-2,3-diamine (1.2 g, 94% yield) as a yellow solid. MS (ESI) calcd. for C₁₈H₁₇N₅: 303.15 m/z, found: 304.15 [M+H]⁺.

### Step 4: Synthesis of 3-(3-{1H,1aH,6H,6aH-cyclopropa[a]inden-3-yl}-5-(pyrazol-1-yl)imidazo[4,5-b]pyridin-2-yl)pyridin-2-amine

To a solution of N2-{1H,1aH,6H,6aH-cyclopropa[a]inden-3-yl}-6-(pyrazol-1-yl)pyridine-2,3-diamine (1.1 g, 3.6 mmol) in AcOH (20 mL) and MeOH (4 mL) was added 2-aminopyridine-3-carbaldehyde (487.1 mg, 3.989 mmol) and sodium perborate (593.2 mg, 7.252 mmol). The resulting mixture was stirred at 70° C. for 3 h. After cooling to room temperature, the reaction was quenched with H₂O (50 mL). The resulting mixture was extracted with ethyl acetate (3×50 mL). The organic layers were combined, dried over Na₂SO₄, filtered and concentrated. The residue was purified by reverse-phase flash column chromatography on C18 silica gel using a gradient of acetonitrile in water (+0.05% NH₄HCO₃) to afford 3-(3-{1H,1aH,6H,6aH-cyclopropa[a]inden-3-yl}-5-(pyrazol-1-yl)imidazo[4,5-b]pyridin-2-yl)pyridin-2-amine (1 g, 68% yield) as a yellow solid. MS (ESI) calcd. for C₂₄H₁₉N₇: 405.17 m/z, found: 406.15 [M+H]⁺.

### Step 5: Synthesis of 3-[2-(2-aminopyridin-3-yl)-5-(pyrazol-1-yl)imidazo[4,5-b]pyridin-3-yl]-1H,1aH,6aH-cyclopropa[a]inden-6-one

To a solution of 3-(3-{1H,1aH,6H,6aH-cyclopropa[a]inden-3-yl}-5-(pyrazol-1-yl)imidazo[4,5-b]pyridin-2-yl)pyridin-2-amine (800 mg, 1.97 mmol) in AcOH (20 mL) was added CrO₃ (591.9 mg, 5.919 mmol). The resulting mixture was stirred overnight at room temperature. The solvent was removed by distillation under vacuum. The residue was purified by reverse-phase flash column chromatography on C18 silica gel using a gradient of acetonitrile in water (+0.05% NH₄HCO₃) to afford 3-[2-(2-aminopyridin-3-yl)-5-(pyrazol-1-yl)imidazo[4,5-b]pyridin-3-yl]-1H,1aH,6aH-cyclopropa[a]inden-6-one (220 mg, 26% yield) as a yellow solid. MS (ESI) calcd. for C₂₄H₁₇N₇O: 419.15 m/z, found: 420.15 [M+H]⁺.

### Step 6: Synthesis of 3-(3-{6-amino-1H,1aH,6H,6aH-cyclopropa[a]inden-3-yl}-5-(pyrazol-1-yl)imidazo[4,5-b]pyridin-2-yl)pyridin-2-amine (Intermediate 252-1)

To a solution of 3-[2-(2-aminopyridin-3-yl)-5-(pyrazol-1-yl)imidazo[4,5-b]pyridin-3-yl]-1H,1aH,6aH-cyclopropa[a]inden-6-one (600 mg, 1.43 mmol) in MeOH (20 mL) was added ammonium formate (902 mg, 14.3 mmol). The resulting mixture was stirred at room temperature for 1 h. To the mixture was added NaBH₃CN (269.7 mg, 4.290 mmol). The resulting mixture was stirred for 2 h at 60° C. The solvent was removed by distillation under vacuum. The resulting mixture was purified by reverse-phase flash column chromatography on C18 silica gel using a gradient of acetonitrile in water (+0.05% NH₄HCO₃) to afford 3-(3-{6-amino-1H,1aH,6H,6aH-cyclopropa[a]inden-3-yl}-5-(pyrazol-1-yl)imidazo[4,5-b]pyridin-2-yl)pyridin-2-amine (Intermediate 252-1) (90 mg, 15% yield) as a yellow solid. MS (ESI) calcd. for C₂₄H₂₀N₈: 420.18 m/z, found: 421.20 [M+H]⁺.

### Example 253: N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-1,3-benzoxazole-7-carboxamide

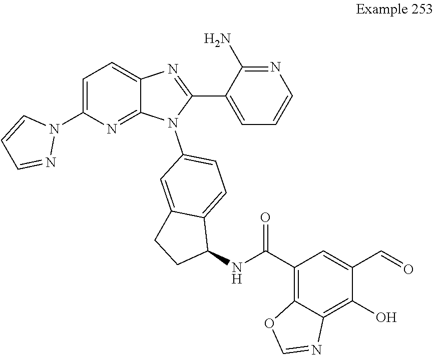 [SMILES: Nc1ncccc1-c1nc2ccc(-n3cccn3)nc2n1-c1ccc2c(c1)CC[C@@H]2NC(=O)c1cc(C=O)c(O)c2ncoc12]

### Synthetic Route:

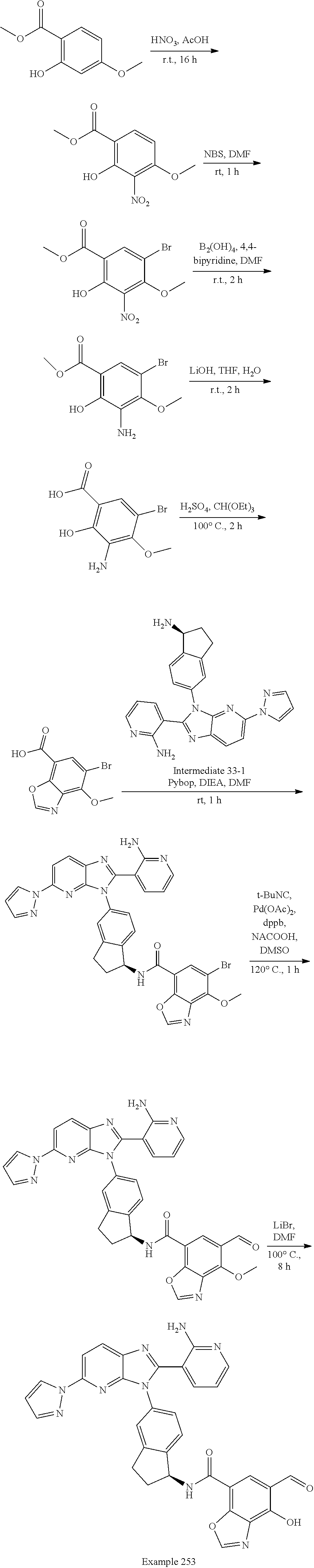

### Step 1: Synthesis of methyl 2-hydroxy-4-methoxy-3-nitrobenzoate

To a solution of methyl 2-hydroxy-4-methoxybenzoate (2 g, 11 mmol) in AcOH (40 mL) were added HNO₃ (2 mL, 45 mmol) dropwise at 0° C. After stirring for 16 h at room temperature, the resulting mixture was diluted with ice/water (100 mL). The precipitated solids were collected by filtration. The solid was purified by silica gel column chromatography, eluted with ethyl acetate in petroleum ether (0˜25%) to afford methyl 2-hydroxy-4-methoxy-3-nitrobenzoate (700 mg, 28% yield) as a light yellow solid. MS (ESI) calcd. for C₉H₉NO₆, 227.04 m/z, found 228.05 [M+H]⁺.

### Step 2: Synthesis of methyl 5-bromo-2-hydroxy-4-methoxy-3-nitrobenzoate

To a stirred solution of methyl 2-hydroxy-4-methoxy-3-nitrobenzoate (700 mg, 3.84 mmol) in N,N-dimethylformamide (10 mL) was added NBS (683.9 mg, 3.842 mmol). The reaction mixture was stirred at room temperature for 1 h. The reaction mixture was quenched by addition of water (20 mL). The aqueous layer was extracted with ethyl acetate (3×30 mL). The combined organic phases were washed with brine (3×30 mL), dried over anhydrous sodium sulfate, filtered and concentrated under reduced pressure. The residue was purified by silica gel column chromatography, eluting with ethyl acetate in petroleum ether (0˜25%) to afford methyl 5-bromo-2-hydroxy-4-methoxy-3-nitrobenzoate (740 mg, 62.92% yield) as a light yellow solid. MS (ESI) calcd. for C₉H₈BrNO₆, 304.95 m/z, found 305.90 [M+H]⁺.

### Step 3: Synthesis of methyl 3-amino-5-bromo-2-hydroxy-4-methoxybenzoate

To a cooled (0° C.) solution of methyl 5-bromo-2-hydroxy-4-methoxy-3-nitrobenzoate (650 mg, 2.12 mmol) in DMF (10 mL) was added B₂(OH)₄ (571.2 mg, 6.372 mmol) and 4,4′-bipyridine (16.6 mg, 0.106 mmol). The reaction mixture was stirred at room temperature for 2 h. After completion of reaction, the reaction mixture was quenched by addition of water (20 mL). The aqueous layer was extracted with ethyl acetate (3×30 mL). The combined organic phases were washed with brine (3×30 mL), dried over anhydrous sodium sulfate, filtered and concentrated under reduced pressure to give methyl 3-amino-5-bromo-2-hydroxy-4-methoxybenzoate (400 mg, 96% yield) as a light brown solid. MS (ESI) calcd. for C₉H₁₀BrNO₄, 274.98 m/z, found 276.00 [M+H]⁺.

### Step 4: Synthesis of 3-amino-5-bromo-2-hydroxy-4-methoxybenzoic acid

To a solution of methyl 3-amino-5-bromo-2-hydroxy-4-methoxybenzoate (200 mg, 0.724 mmol) in THF (10 mL) was added a solution of LiOH (52.1 mg, 2.17 mmol) in water (2.5 mL) at 0° C. The obtained solution was stirred for 2 h at room temperature. The THF was removed under reduced pressure. After concentration, the obtained solution was acidified to pH=6 with HCl (2 N, aq.). The precipitated solids were collected by filtration to afford 3-amino-5-bromo-2-hydroxy-4-methoxybenzoic acid (100 mg, 53% yield) as a light yellow solid. MS (ESI) calcd. for C₈H₈BrNO₄, 260.96 m/z, found 261.95 [M+H]⁺.

### Step 5: Synthesis of 5-bromo-4-methoxy-1,3-benzoxazole-7-carboxylic acid

A solution of 3-amino-5-bromo-2-hydroxy-4-methoxybenzoic acid (100 mg, 0.382 mmol) in triethyl orthoformate (3 mL) was stirred for 2 h at 100° C. The reaction mixture was quenched by addition of water (2 mL). The mixture was concentrated directly to obtain 5-bromo-4-methoxy-1,3-benzoxazole-7-carboxylic acid (100 mg, 96% yield) as a light brown solid. MS (ESI) calcd. for C₉H₆BrNO₄, 270.95, m/z, found 271.95 [M+H]⁺.

### Step 6: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-5-bromo-4-methoxy-1,3-benzoxazole-7-carboxamide

To a suspension of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (180 mg, 0.441 mmol) in DMF (4 mL) was added 5-bromo-4-methoxy-1,3-benzoxazole-7-carboxylic acid (120 mg, 0.441 mmol), PyBOP (253 mg, 0.485 mmol) and N,N-diisopropylethylamine (171 mg, 1.323 mmol). The resulting mixture was stirred at room temperature for 1 h. The mixture was purified directly by reverse-phase flash column chromatography on C18 silica gel using a gradient of acetonitrile in water (+0.05% NH₄HCO₃) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-5-bromo-4-methoxy-1,3-benzoxazole-7-carboxamide (210 mg, 72% yield) as a light yellow solid. MS (ESI) calcd. for C₃₂H₂₄BrN₉O₃, 661.12, m/z, found 662.15 [M+H]⁺.

### Step 7: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-methoxy-1,3-benzoxazole-7-carboxamide

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-5-bromo-4-methoxy-1,3-benzoxazole-7-carboxamide (100 mg, 0.134 mmol) in DMSO (10 mL) was added Pd(OAc)₂ (6.02 mg, 0.027 mmol), dppb (114 mg, 0.268 mmol), sodium formate (27.4 mg, 0.402 mmol) and 2-isocyano-2-methylpropane (33.5 mg, 0.402 mmol). The obtained mixture was stirred for 1 h at 120° C. under a nitrogen atmosphere. The residue was purified directly by reverse-phase flash column chromatography on C18 silica gel using a gradient of acetonitrile in water (0.05% NH₄HCO₃) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-methoxy-1,3-benzoxazole-7-carboxamide (30 mg, 37% yield) as a light yellow solid. MS (ESI) calcd. for C₃₃H₂₅N₉O₄, 611.20, m/z, found 612.25 [M+H]⁺.

### Step 8: Synthesis of N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-1,3-benzoxazole-7-carboxamide

To a suspension of N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-methoxy-1,3-benzoxazole-7-carboxamide (30 mg, 0.049 mmol) in DMF (2 mL) was added LiBr (42.6 mg, 0.490 mmol). The obtained mixture was stirred for 8 h at 100° C. The residue was purified directly by Prep-HPLC on a XSelect CSH Fluoro Phenyl column using a gradient of acetonitrile in water (+10 mmol/L ammonium bicarbonate) to afford N-[(1S)-5-[2-(2-aminopyridin-3-yl)-5-(pyrazol-1-yl)imidazo[4,5-b]pyridin-3-yl]-2,3-dihydro-1H-inden-1-yl]-5-formyl-4-hydroxy-1,3-benzoxazole-7-carboxamide (Example 253) (7.4 mg, 25% yield) as a light yellow solid. MS (ESI) calcd. for C₃₂H₂₃N₉O₄, 597.19, m/z, found 598.25 [M+H]⁺. ¹H NMR (400 MHz, DMSO-d₆) δ (ppm): 10.01 (s, 1H), 8.38-8.44 (m, 3H), 8.09-8.11 (m, 1H), 8.05-8.06 (m, 1H), 7.97-8.00 (m, 1H), 7.85 (s, 1H), 7.31-7.47 (m, 4H), 6.56-6.61 (m, 2H), 5.64-5.69 (m, 1H), 3.07-3.13 (m, 1H), 2.94-2.98 (m, 1H), 2.65-2.67 (m, 1H), 2.12-2.17 (m, 1H).

### Example 254: 5-[(1S)-5-[2-(2-aminopyridin-3-yl)-5-(pyrazol-1-yl)imidazo[4,5-b]pyridin-3-yl]-2,3-dihydro-1H-inden-1-yl]-2,6-dioxo-3,4-dihydro-1H-1,5-benzodiazocine-10-carbaldehyde

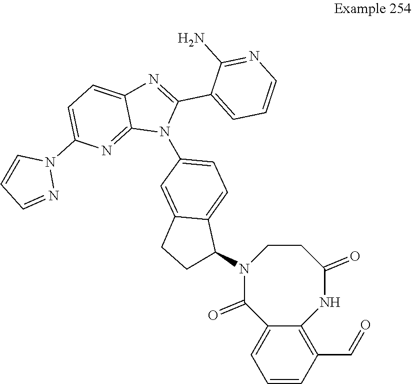 [SMILES: Nc1ncccc1-c1nc2ccc(-n3cccn3)nc2n1-c1ccc2c(c1)CC[C@@H]2N1CCC(=O)Nc2c(C=O)cccc2C1=O]

### Synthetic Route:

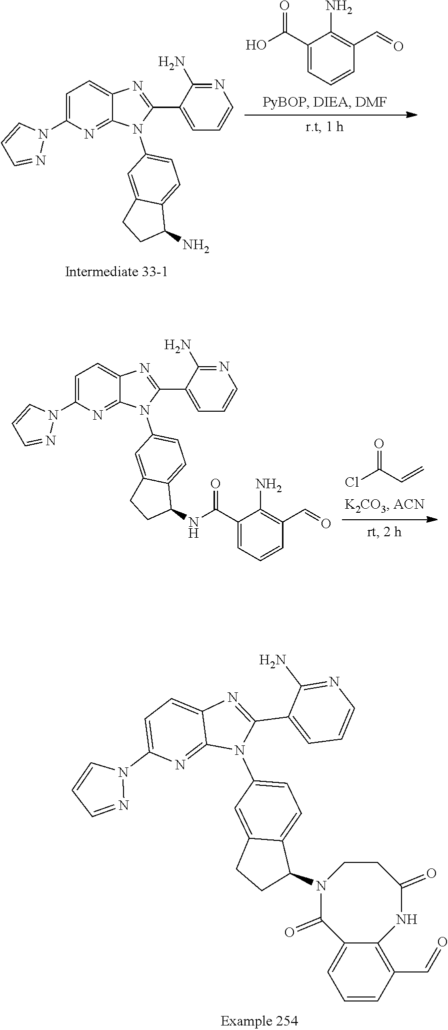

### Step 1: Synthesis of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-3-formylbenzamide

To a suspension of 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (494.7 mg, 1.211 mmol) in DMF (8 mL) was added 2-amino-3-formylbenzoic acid (200 mg, 1.21 mmol), PyBOP (630.2 mg, 1.211 mmol) and N,N-diisopropylethylamine (469.6 mg, 3.633 mmol). The reaction mixture was stirred at room temperature for 1 h. The mixture was purified directly by reverse-phase flash column chromatography on C18 silica gel using a gradient of acetonitrile in water (+0.05% NH₄HCO₃) to afford 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-3-formylbenzamide (200 mg, 30% yield) as a green yellow solid. MS (ESI) calcd. for C₃₁H₂₅N₉O₂, 555.21 m/z, found 556.25 [M+H]⁺.

### Step 2: Synthesis of 5-[(1S)-5-[2-(2-aminopyridin-3-yl)-5-(pyrazol-1-yl)imidazo[4,5-b]pyridin-3-yl]-2,3-dihydro-1H-inden-1-yl]-2,6-dioxo-3,4-dihydro-1H-1,5-benzodiazocine-10-carbaldehyde (Example 254)

To a cooled (0° C.) suspension of 2-amino-N-[(1S)-5-[2-(2-aminopyridin-3-yl)-5-(pyrazol-1-yl)imidazo[4,5-b]pyridin-3-yl]-2,3-dihydro-1H-inden-1-yl]-3-formylbenzamide (80 mg, 0.14 mmol) in ACN (5 mL) was added K₂CO₃ (39.8 mg, 0.288 mmol) and acryloyl chloride (13.0 mg, 0.144 mmol). The reaction mixture was stirred at room temperature for 2 h. The residue was purified directly by reverse-phase flash column chromatography on C18 silica gel using a gradient of acetonitrile in water (+0.05% TFA) to afford 5-[(1S)-5-[2-(2-aminopyridin-3-yl)-5-(pyrazol-1-yl)imidazo[4,5-b]pyridin-3-yl]-2,3-dihydro-1H-inden-1-yl]-2,6-dioxo-3,4-dihydro-1H-1,5-benzodiazocine-10-carbaldehyde (Example 254) (19.5 mg, 21% yield) as a light yellow solid. MS (ESI) calcd. for C₃₄H₂₇N₉O₃, 609.22 m/z, found 610.15 [M+H]⁺. ¹H NMR (400 MHz, DMSO-d₆) δ (ppm): 9.93 (s, 1H), 8.73-8.84 (m, 1H), 8.43-8.45 (m, 1H), 8.31-8.39 (m, 2H), 8.22-8.31 (m, 2H), 7.99-8.06 (m, 1H), 7.94-7.97 (m, 1H), 7.87-7.91 (m, 1H), 7.81-7.82 (m, 1H), 7.74-7.80 (m, 1H), 7.29-7.32 (m, 2H), 7.14-7.23 (m, 1H), 6.79-6.93 (m, 1H), 6.68-6.73 (m, 1H), 6.52-6.55 (m, 1H), 5.45-5.73 (m, 1H), 4.24-4.28 (m, 2H), 2.92-3.01 (m, 1H), 2.81-2.89 (m, 1H), 2.41-2.47 (m, 1H), 2.03-2.30 (m, 3H).

### Example 255: (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-fluoro-3-formyl-4-hydroxybenzamide

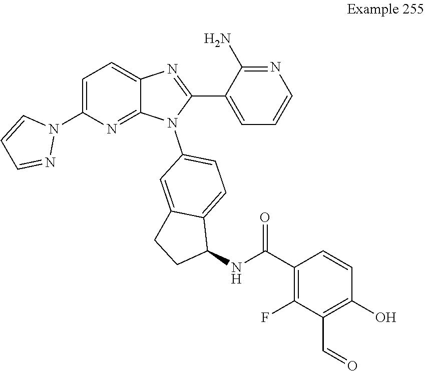 [SMILES: Nc1ncccc1-c1nc2ccc(-n3cccn3)nc2n1-c1ccc2c(c1)CC[C@@H]2NC(=O)c1ccc(O)c(C=O)c1F]

### Synthetic Route:

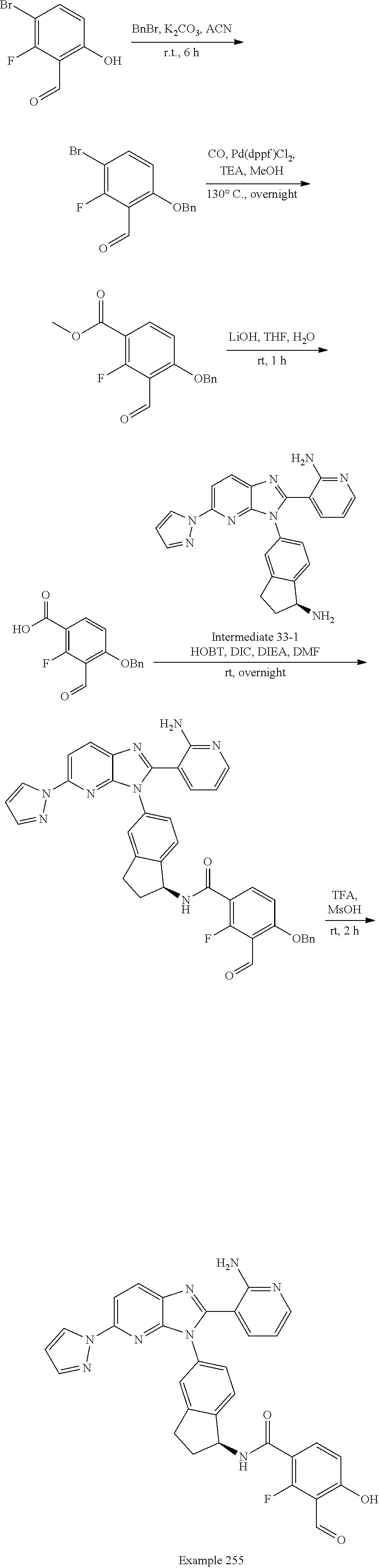

### Step 1: Synthesis of 6-(benzyloxy)-3-bromo-2-fluorobenzaldehyde

To a cooled (0° C.) solution of 3-bromo-2-fluoro-6-hydroxybenzaldehyde (1 g, 4.57 mmol) in ACN (30 mL) was added K₂CO₃ (1.26 g, 9.13 mmol) followed by benzyl bromide (1.17 g, 6.85 mmol). The resulting mixture was stirred at room temperature for 6 h. The reaction was quenched with H₂O (50 mL). The resulting mixture was extracted with ethyl acetate (3×50 mL). The organic layers were combined, dried over Na₂SO₄, and concentrated under vacuum to obtain 6-(benzyloxy)-3-bromo-2-fluorobenzaldehyde (800 mg, 56.66% yield) as a white liquid. MS (ESI) calcd. for C₁₄H₁₀BrFO₂: 307.98 m/z, found: 308.95 [M+H]⁺.

### Step 2: Synthesis of methyl 4-(benzyloxy)-2-fluoro-3-formylbenzoate

A mixture of 6-(benzyloxy)-3-bromo-2-fluorobenzaldehyde (400 mg, 1.29 mmol), Pd(dppf)Cl₂ (189.4 mg, 0.259 mmol), triethylamine (392.8 mg, 3.882 mmol), and MeOH (50 mL) was stirred overnight at 130° C. under a CO (g) (30 bar) atmosphere. The resulting mixture was allowed to cool to room temperature, depressurized and concentrated under vacuum. The residue obtained was purified by silica gel column chromatography (0-50% ethyl acetate in hexane) to obtain methyl 4-(benzyloxy)-2-fluoro-3-formylbenzoate (168 mg, 45% yield). MS (ESI) calcd. For C₁₆H₁₃FO₄: 288.08 m/z, found: 289.10 [M+H]⁺.

### Step 3: Synthesis of 4-(benzyloxy)-2-fluoro-3-formylbenzoic acid

To a solution of methyl 4-(benzyloxy)-2-fluoro-3-formylbenzoate (142 mg, 0.493 mmol) in THF (5 mL) was added a solution of LiOH (59 mg, 2.5 mmol) in H₂O (2.5 mL). The resulting mixture was stirred at room temperature for 1 h. The reaction was quenched with H₂O (50 mL). The mixture was brought to pH 6 with HCl (1M, aq). The resulting mixture was extracted with ethyl acetate (3×50 mL). The organic layers were combined, dried over Na₂SO₄ and concentrated under vacuum to obtain 4-(benzyloxy)-2-fluoro-3-formylbenzoic acid (100.7 mg, 74% yield) as a white solid. MS (ESI) calcd. for C₁₅H₁₁FO₄: 274.06 m/z, found: 272.95 [M−H]⁻.

### Step 4: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-2-fluoro-3-formylbenzamide

To a solution of 4-(benzyloxy)-2-fluoro-3-formylbenzoic acid (100.7 mg, 0.367 mmol) and 3-{3-[(1S)-1-amino-2,3-dihydro-1H-inden-5-yl]-5-(pyrazol-1-yl)imidazo[4,5-b]pyridin-2-yl}pyridin-2-amine (Intermediate 33-1) (100 mg, 0.245 mmol) in DMF (10 mL) was added HOBT (39.7 mg, 0.294 mmol), DIC (37.0 mg, 0.294 mmol) and N,N-diisopropylethylamine (63.3 mg, 0.490 mmol). The resulting mixture was purified by reverse-phase flash column chromatography on C18 silica gel using a gradient of acetonitrile in water (+0.05% TFA) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-4-(benzyloxy)-2-fluoro-3-formylbenzamide (146 mg, 90% yield) as an off-white solid. MS (ESI) calcd. for C₃₈H₂₉FN₈O₃: 664.23 m/z, found: 665.15 [M+H]⁺.

### Step 5: Synthesis of (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-fluoro-3-formyl-4-hydroxybenzamide (Example 255)

To a solution of N-[(1S)-5-[2-(2-aminopyridin-3-yl)-5-(pyrazol-1-yl)imidazo[4,5-b]pyridin-3-yl]-2,3-dihydro-1H-inden-1-yl]-4-(benzyloxy)-2-fluoro-3-formylbenzamide (174 mg, 0.262 mmol) in TFA (12.5 mL) was added methanesulfonic acid (5.5 mL). The resulting mixture was stirred at room temperature for 2 h. The reaction mixture was concentrated under vacuum and the residue was quenched with H₂O (50 mL). The mixture was basified to pH 8 with saturated aqueous sodium bicarbonate. The resulting mixture was extracted with ethyl acetate (3×50 mL). The organic layers were combined, dried over sodium sulfate and concentrated under vacuum. The residue was then purified by Prep-HPLC on a XBridge Prep OBD C18 Column using a gradient of acetonitrile in water (+10 mmol/L NH₄HCO₃) to afford (S)—N-(5-(2-(2-aminopyridin-3-yl)-5-(1H-pyrazol-1-yl)-3H-imidazo[4,5-b]pyridin-3-yl)-2,3-dihydro-1H-inden-1-yl)-2-fluoro-3-formyl-4-hydroxybenzamide (Example 255) (9.0 mg, 6% yield) as an off-white solid. MS (ESI) calcd. for C₃₁H₂₃FN₈O₃: 574.19 m/z, found: 575.05 [M+H]⁺. ¹H NMR (300 MHz, DMSO-d₆) δ (ppm): 9.43-10.41 (m, 1H), 8.15-8.46 (m, 2H), 7.87-8.12 (m, 2H), 7.74-7.86 (m, 1H), 7.51-7.73 (m, 1H), 7.35-7.44 (m, 2H), 7.19-7.34 (m, 2H), 6.22-6.94 (m, 3H), 5.42-5.71 (m, 1H), 2.67-3.14 (m, 2H), 2.23-2.47 (m, 1H), 1.84-2.18 (m, 1H). ¹⁹F NMR (282 MHz, DMSO-d₆) δ (ppm): −117.25.

### Example 256: N-[(1R,2R*)-5-[2-(2-aminopyridin-3-yl)-5-(pyrazol-1-yl)imidazo[4,5-b]pyridin-3-yl]-2-fluoro-2,3-dihydro-1H-inden-1-yl]-3-formyl-4-hydroxybenzamide

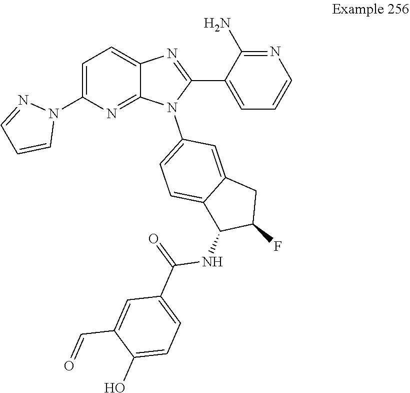 [SMILES: Nc1ncccc1-c1nc2ccc(-n3cccn3)nc2n1-c1ccc2c(c1)C[C@@H](F)[C@@H]2NC(=O)c1ccc(O)c(C=O)c1]

Example 256 was prepared in a manner analogous to Example 12 using Intermediate 256-1 in place of Intermediate 1-3, Intermediate 5-1 in place of Intermediate 12-1 and dichloromethane/TFA (10:1) in place of TFA/methanesulfonic acid. *Note that the stereochemistry of the fluorine is assumed. MS (ESI) calcd. for C₃₁H₂₃FN₈O₃: 574.19 m/z, found: 575.10 [M+H]⁺. ¹H-NMR (400 MHz, DMSO-d₆) δ (ppm): 10.27 (s, 1H), 8.40-8.48 (m, 1H), 8.35-8.39 (m, 2H), 8.12-8.18 (m, 1H), 7.98-8.05 (m, 2H), 7.80-7.85 (m, 2H), 7.48-7.55 (m, 1H), 7.35-7.46 (m, 2H), 7.05-7.13 (m, 1H), 6.78-6.85 (m, 1H), 6.55-6.60 (m, 1H), 5.69-5.80 (m, 1H), 5.35-5.55 (m, 1H), 3.15-3.50 (m, 2H). ¹⁹F NMR (376 MHz, DMSO-d₆) δ (ppm): −192.42. (TFA salt).

### Intermediate 256-1: 3-(3-((1R,2R*)-1-amino-2-fluoro-2,3-dihydro-1H-inden-5-yl)-5-(1H-pyrazol-1-yl)-3H-imidazo[4,5-b]pyridin-2-yl)pyridin-2-amine and Intermediate 256-2: 3-(3-((1R,2S*)-1-amino-2-fluoro-2,3-dihydro-1H-inden-5-yl)-5-(1H-pyrazol-1-yl)-3H-imidazo[4,5-b]pyridin-2-yl)pyridin-2-amine

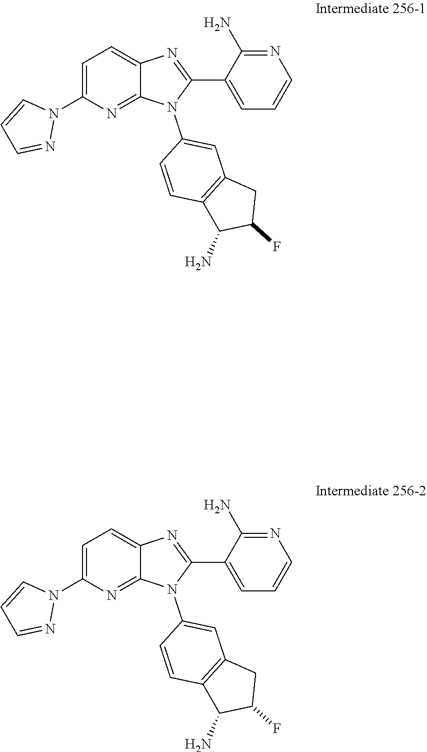 [SMILES: Nc1ncccc1-c1nc2ccc(-n3cccn3)nc2n1-c1ccc2c(c1)C[C@@H](F)[C@@H]2N ; Nc1ncccc1-c1nc2ccc(-n3cccn3)nc2n1-c1ccc2c(c1)C[C@H](F)[C@@H]2N]

### Synthetic Route:

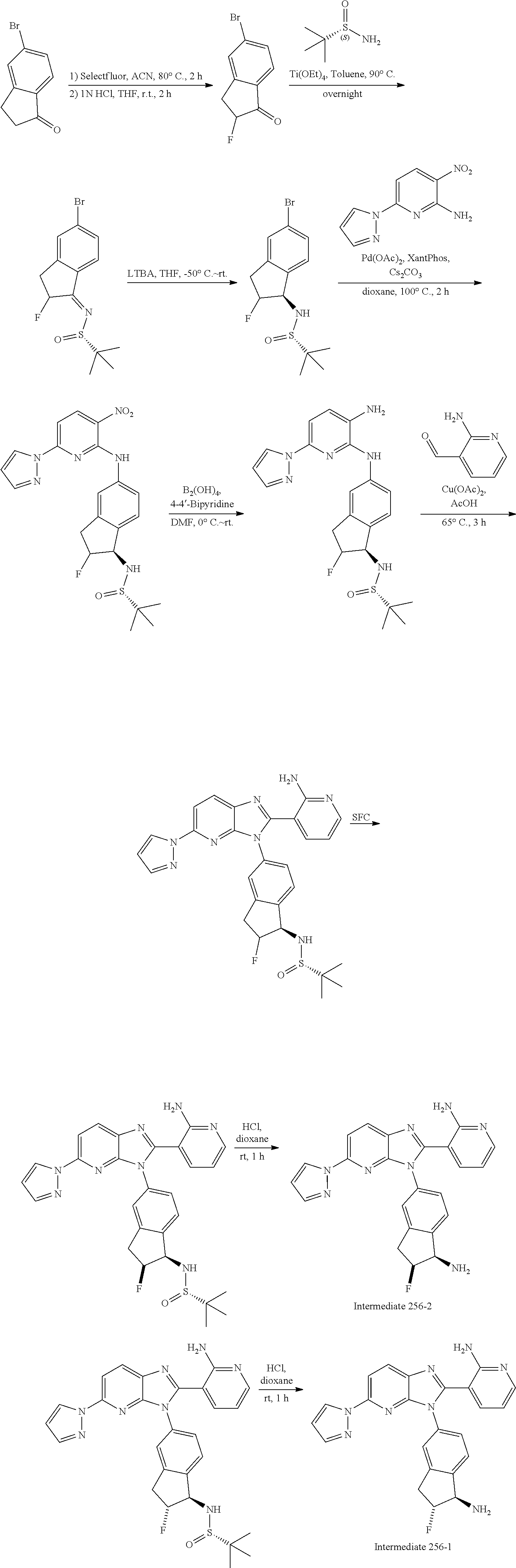

### Step 1: Synthesis of 5-bromo-2-fluoro-2,3-dihydro-1H-inden-1-one

To a solution of 5-bromo-1-indanone (5.00 g, 23.8 mmol) in methanol (50 mL) was added SelectFluor (10.0 g, 28.2 mmol) and the resulting mixture was stirred under reflux for 2 hours. The reaction mixture was filtered through Celite, and the filtrate was concentrated under reduced pressure. The resulting residue was dissolved in tetrahydrofuran (50 mL), and 1 N hydrochloric acid (50 mL) was added followed by stirring at room temperature for 3 hours. To the reaction mixture, a 2 N aqueous sodium hydroxide solution (50 mL) was added, and the mixture was diluted with a saturated aqueous sodium bicarbonate solution. The mixture was extracted with ethyl acetate. The organic layer was washed with brine, dried over anhydrous magnesium sulfate, and then concentrated under reduced pressure to afford 5-bromo-2-fluoro-2,3-dihydroinden-1-one (4.0 g, 74% yield) as a white solid. MS (ESI) calcd. for C₉H₆BrFO, 227.96 m/z, found: 229.00 [M+H]⁺.

### Step 2: Synthesis of (S)—N—((Z)-5-bromo-2-fluoro-2,3-dihydro-1H-inden-1-ylidene)-2-methylpropane-2-sulfinamide

To a solution of 5-bromo-2-fluoro-2,3-dihydroinden-1-one (3.0 g, 13 mmol) in toluene (5 mL) was added (S)-2-methylpropane-2-sulfinamide (1.90 g, 15.7 mmol) and Ti(OEt)₄ (5.08 g, 22.2 mmol). The mixture was stirred at 90° C. for 2 h. After cooling to room temperature, the reaction was quenched by the addition of 2M Rochelle's salt (30 mL). The resulting mixture was diluted with brine (50 mL) and extracted with ethyl acetate (3×50 mL). The combined organic layers were dried over anhydrous Na₂SO₄. After filtration, the filtrate was concentrated under reduced pressure. The residue was purified by silica gel column chromatography using a gradient of ethyl acetate in petroleum ether to afford (S)—N-(5-bromo-2-fluoro-2,3-dihydro-1H-inden-1-ylidene)-2-methylpropane-2-sulfinamide (3.0 g, 69% yield) as a yellow solid. MS (ESI) calcd. for C₁₃H₁₅BrFNOS: 331.00 m/z, found: 332.10 [M+H]⁺. Note that the (S)-applies to the sulfur stereocenter and not the C—F bond for this and all instances vida infra.

### Step 3: Synthesis of (S)—N-((1R)-5-bromo-2-fluoro-2,3-dihydro-1H-inden-1-yl)-2-methylpropane-2-sulfinamide

To a cooled (−50° C.) solution of (S)—N—((Z)-5-bromo-2-fluoro-2,3-dihydro-1H-inden-1-ylidene)-2-methylpropane-2-sulfinamide (3.0 g, 9.0 mmol) was added LTBA (3.90 g, 15.4 mmol) and the resulting mixture was stirred at room temperature for 2 h. The reaction was quenched by the addition of 2M Rochelle's salt (30 mL). The mixture was diluted with brine (50 mL) and extracted with ethyl acetate (3×50 mL). The combined organic layers were dried over anhydrous Na₂SO₄. After filtration, the filtrate was concentrated under reduced pressure. The residue was purified by silica gel column chromatography using a gradient of ethyl acetate in petroleum ether to afford (S)—N-((1R)-5-bromo-2-fluoro-2,3-dihydro-1H-inden-1-yl)-2-methylpropane-2-sulfinamide (2.0 g, 66% yield) as a yellow solid. MS (ESI) calcd. for C₁₃H₁₇BrFNOS: 333.02 m/z, found: 334.10 [M+H]⁺.

### Step 4: Synthesis of (S)—N-((1R)-2-fluoro-5-((3-nitro-6-(1H-pyrazol-1-yl)pyridin-2-yl)amino)-2,3-dihydro-1H-inden-1-yl)-2-methylpropane-2-sulfinamide

A mixture of (S)—N-((1R)-5-bromo-2-fluoro-2,3-dihydro-1H-inden-1-yl)-2-methylpropane-2-sulfinamide (2.0 g, 6.0 mmol), 3-nitro-6-(pyrazol-1-yl)pyridin-2-amine (1.35 g, 6.58 mmol), Pd(OAc)₂ (0.13 g, 0.60 mmol), Cs₂CO₃ (5.86 g, 18.0 mmol) and XantPhos (0.35 g, 0.60 mmol) in dioxane (4.0 mL) was stirred at 100° C. for 2 h under nitrogen atmosphere. The reaction was quenched with water (100 mL) and the mixture was extracted with ethyl acetate (3×100 mL). The combined organic extracts were washed with brine (100 mL) and dried over anhydrous Na₂SO₄. After filtration, the filtrate was concentrated under reduced pressure. The crude product was purified by silica gel column chromatography using a gradient of methanol in dichloromethane to afford (S)—N-((1R)-2-fluoro-5-((3-nitro-6-(1H-pyrazol-1-yl)pyridin-2-yl)amino)-2,3-dihydro-1H-inden-1-yl)-2-methylpropane-2-sulfinamide (2.5 g, 91% yield) as a yellow solid. MS (ESI) calcd. for C₂₁H₂₃FN₆O₃S, 458.15 m/z, found: 459.10 [M+H]⁺.

### Step 5: Synthesis of (S)—N-((1R)-5-((3-amino-6-(1H-pyrazol-1-yl)pyridin-2-yl)amino)-2-fluoro-2,3-dihydro-1H-inden-1-yl)-2-methylpropane-2-sulfinamide

To a solution of (S)—N-[(1R)-2-fluoro-5-{[3-nitro-6-(pyrazol-1-yl)pyridin-2-yl]amino}-2,3-dihydro-1H-inden-1-yl]-2-methylpropane-2-sulfinamide (2.5 g, 5.5 mmol) in DMF (30 mL) was added B₂(OH)₄ (1.47 g, 16.4 mmol) and 4-(pyridin-4-yl)pyridine (25.0 mg, 0.163 mmol). The resulting mixture was stirred at room temperature for 1 h. The reaction was quenched by the addition of water (200 mL) and then the mixture was extracted with ethyl acetate (3×100 mL). The combined organic extracts were washed with brine (100 mL) and dried over anhydrous Na₂SO₄. After filtration, the filtrate was concentrated under reduced pressure to afford (S)—N-((1R)-5-((3-amino-6-(1H-pyrazol-1-yl)pyridin-2-yl)amino)-2-fluoro-2,3-dihydro-1H-inden-1-yl)-2-methylpropane-2-sulfinamide (2.0 g, 86% yield) as a yellow solid. MS (ESI) calcd. for C₂₁H₂₅FN₆OS: 428.17 m/z. found: 429.15 [M+H]⁺.

### Step 6: Synthesis of (S)—N-((1R)-5-(2-(2-aminopyridin-3-yl)-5-(1H-pyrazol-1-yl)-3H-imidazo[4,5-b]pyridin-3-yl)-2-fluoro-2,3-dihydro-1H-inden-1-yl)-2-methylpropane-2-sulfinamide

To a solution of (S)—N-[(1R)-5-{[3-amino-6-(pyrazol-1-yl)pyridin-2-yl]amino}-2-fluoro-2,3-dihydro-1H-inden-1-yl]-2-methylpropane-2-sulfinamide (2.0 g, 4.7 mmol) and 2-aminopyridine-3-carbaldehyde (0.68 g, 5.6 mmol) in AcOH (60 mL) was added Cu(OAc)₂ (171 mg, 0.94 mmol). The mixture was stirred at 65° C. for 1.0 h. After cooling to room temperature, the mixture was concentrated and the residue obtained was purified by reverse phase flash column chromatography on C18 silica gel using a gradient of acetonitrile in water (+0.05 mmol/L NH₄HCO₃) to afford (S)—N-((1R)-5-(2-(2-aminopyridin-3-yl)-5-(1H-pyrazol-1-yl)-3H-imidazo[4,5-b]pyridin-3-yl)-2-fluoro-2,3-dihydro-1H-inden-1-yl)-2-methylpropane-2-sulfinamide (1.47 g, 59% yield). MS (ESI) calcd. for C₂₇H₂₇FN₈OS, 530.20 m/z, found: 531.25 [M+H]⁺. The two diastereomers were separated by chiral SFC on a (S,S)-Whelk-O 1 5 μm Kromasil column using a 2:1 mix of CO₂ and [acetonitrile/methanol 4:1]. The first eluting peak was carried through step 7 below to afford Intermediate 256-2 and the second eluting peak was converted to Intermediate 256-1.

### Step 7: Synthesis of 3-(3-((1R)-1-amino-2-fluoro-2,3-dihydro-1H-inden-5-yl)-5-(1H-pyrazol-1-yl)-3H-imidazo[4,5-b]pyridin-2-yl)pyridin-2-amine (Intermediates 256-1 and 256-2)

The two diastereomers of (S)—N-((1R)-5-(2-(2-aminopyridin-3-yl)-5-(1H-pyrazol-1-yl)-3H-imidazo[4,5-b]pyridin-3-yl)-2-fluoro-2,3-dihydro-1H-inden-1-yl)-2-methylpropane-2-sulfinamide (500 mg, 0.754 mmol) were dissolved separately in 4N HCl in dioxane (8 mL) and the resulting mixtures were stirred at room temperature for 1 h under nitrogen atmosphere. The solvent was removed under vacuum to afford 3-(3-((1R)-1-amino-2-fluoro-2,3-dihydro-1H-inden-5-yl)-5-(1H-pyrazol-1-yl)-3H-imidazo[4,5-b]pyridin-2-yl)pyridin-2-amine (400 mg, crude) as a light yellow solid. MS (ESI) calcd. For C₂₃H₁₉FN₈: 426.17 m/z, found: 427.30 [M+H]⁺.

### Example 257: N-[(1R,2S*)-5-[2-(2-aminopyridin-3-yl)-5-(pyrazol-1-yl)imidazo[4,5-b]pyridin-3-yl]-2-fluoro-2,3-dihydro-1H-inden-1-yl]-3-formyl-4-hydroxybenzamide

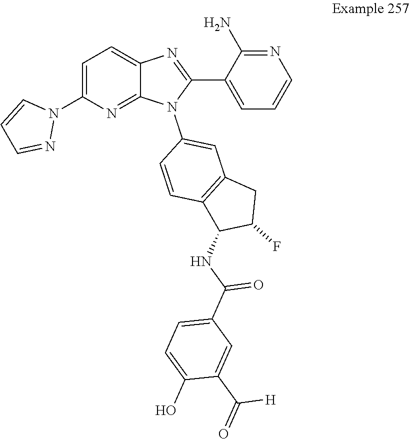 [SMILES: Nc1ncccc1-c1nc2ccc(-n3cccn3)nc2n1-c1ccc2c(c1)C[C@H](F)[C@@H]2NC(=O)c1ccc(O)c(C=O)c1]

Example 257 was prepared in a manner analogous to Example 12 using Intermediate 256-2 in place of Intermediate 1-3, Intermediate 5-1 in place of Intermediate 12-1 and dichloromethane/TFA (10:1) in place of TFA/methanesulfonic acid. *Note that the stereochemistry of the fluorine is assumed. MS (ESI) calcd. for C₃₁H₂₃FN₈O₃, 574.19 m/z, found 575.20 [M+H]⁺. ¹H NMR (400 MHz, DMSO-d₆) δ (ppm) 10.31-10.32 (m, 1H), 8.40-8.44 (m, 2H), 8.28-8.38 (m, 1H), 8.15-8.17 (m, 2H), 8.00-8.10 (m, 1H), 7.80-7.83 (m, 1H), 7.72-7.74 (m, 1H), 7.51-7.52 (m, 1H), 7.40-7.44 (m, 2H), 7.07-7.11 (m, 1H) 6.77-6.80 (m, 1H), 6.57-6.60 (m, 1H), 5.52-5.73 (m, 1H), 5.39-5.40 (m, 1H), 3.10-3.27 (m, 2H). ¹⁹F NMR (282 MHz, DMSO-d₆) δ (ppm): −179.30. (TFA salt).

### Example 258: N-((1R,2R*)-5-(2-(2-aminopyridin-3-yl)-5-(1H-pyrazol-1-yl)-3H-imidazo[4,5-b]pyridin-3-yl)-2-fluoro-2,3-dihydro-1H-inden-1-yl)-2-fluoro-5-formyl-4-hydroxybenzamide

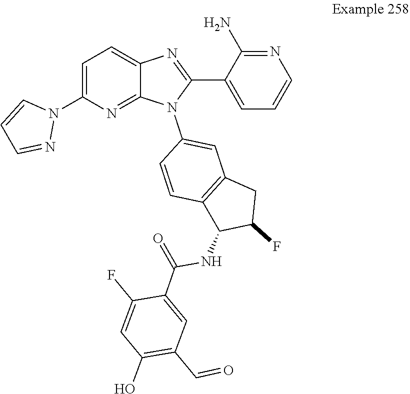 [SMILES: Nc1ncccc1-c1nc2ccc(-n3cccn3)nc2n1-c1ccc2c(c1)C[C@@H](F)[C@@H]2NC(=O)c1cc(C=O)c(O)cc1F]

Example 258 was prepared in a manner analogous to Example 12 using Intermediate 256-1 in place of Intermediate 1-3 and Intermediate 16-1 in place of Intermediate 12-1. *Note that the stereochemistry of the C—F bond is assumed. MS (ESI) calcd. for C₃₁H₂₂F₂N₈O₃: 592.18 m/z, found: 593.15 [M+H]⁺. ¹H-NMR (400 MHz, DMSO-d6) δ (ppm): 10.20 (s, 1H), 8.36-8.38 (m, 2H), 8.07-8.10 (m, 1H), 8.01-8.02 (m, 1H), 7.95-7.97 (m, 1H), 7.80-7.81 (m, 1H), 7.38-7.46 (m, 3H), 7.26-7.28 (m, 1H), 6.70-6.73 (m, 1H), 6.54-6.55 (m, 1H), 6.43-6.46 (m, 1H), 5.69-5.77 (m, 1H), 5.41-5.57 (m, 1H), 3.14-3.39 (m, 2H). ¹⁹F-NMR (400 MHz, DMSO-d6) δ (ppm): −193.14, −102.55.

### Example 259: N-((1R,2S*)-5-(2-(2-aminopyridin-3-yl)-5-(1H-pyrazol-1-yl)-3H-imidazo[4,5-b]pyridin-3-yl)-2-fluoro-2,3-dihydro-1H-inden-1-yl)-2-fluoro-5-formyl-4-hydroxybenzamide

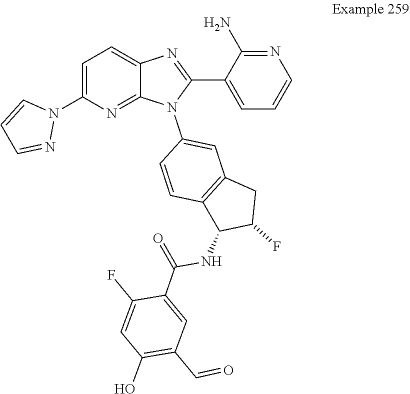 [SMILES: Nc1ncccc1-c1nc2ccc(-n3cccn3)nc2n1-c1ccc2c(c1)C[C@H](F)[C@@H]2NC(=O)c1cc(C=O)c(O)cc1F]

Example 259 was prepared in a manner analogous to Example 12 using Intermediate 256-2 in place of Intermediate 1-3 and Intermediate 16-1 in place of Intermediate 12-1. *Note that the stereochemistry of the C—F bond is assumed. MS (ESI) calcd. for C₃₁H₂₂F₂N₈O₃, 592.18 m/z, found 593.15 [M+H]⁺. ¹H NMR (400 MHz, DMSO-d₆) δ (ppm): 10.21-10.22 (m, 1H), 8.39-8.44 (m, 2H), 8.00-8.08 (m, 3H), 7.73-7.75 (m, 1H), 7.46-7.51 (m, 1H), 7.44-7.46 (m, 1H), 7.40-7.42 (m, 2H), 6.81-6.87 (m, 1H), 6.75-6.79 (m, 1H), 6.56-6.57 (m, 1H), 5.37-5.62 (m, 2H), 3.52-3.55 (m, 1H), 3.13-3.20 (m, 1H). ¹⁹F NMR (376 MHz, DMSO-d₆) δ (ppm): −101.623, −179.398. (TFA salt).

### Example 260: AKT1 Inhibition Data

Antiproliferative effects in LAPC4 cells. LAPC4 prostate cancer cells (Klein, K. A. et al. Nat Med 1997, 3, 402-408), which express the AKT1 E17K allele, were grown in IMDM media (Hyclone) supplemented with 12% FBS+1% P/S. To assess compound effects on growth, 1,000 cells/well were seeded in 384 well assay plates, incubated with compound dilutions dissolved in DMSO. After 72 h, intracellular ATP content was assessed with CellTiter Glo reagent (Promega) according to the manufacturer's instructions. IC₅₀ values were calculated by fitting luminescence values to a log(inhibitor) vs. response Hill equation.

Determining live cell AKT1 E17K, AKT1 WT, and AKT2 target engagement by NanoBRET competitive probe displacement. Live cell target engagement assays were performed as described (Vasta, J. D. et al. Cell Chem Biol 2018, 25(11), 206-214). Briefly, HEK293 cells were transfected with plasmids encoding kinase-NanoLuciferase fusion proteins overnight. Cells were then treated with serial dilutions of compound and an ˜EC₅₀ concentration of fluorescently-tagged ATP-competitive tracer (Promega). After incubation for 2 h at 37° C., luciferase substrate and extracellular luciferase inhibitor were added to all wells, and luminescent intensity at 460 nm and 600 nm were measured on a multimode plate-reader. The ratio of E₆₀₀/E₄₆₀ was calculated to give the tracer engagement signal (BRET). IC₅₀ values were calculated by fitting BRET values to a log(inhibitor) vs. response Hill equation.

Table 2 provides IC₅₀ values for selected compounds of the present disclosure, with compounds having a IC₅₀<10 nM as A; 10 nM≤IC₅₀<100 nM as B; 100≤IC₅₀<2000 nM as C; and IC₅₀≥2000 nM as D. n.d. refers to not determined.

TABLE 2

| | LAPC4 | AKT1 | AKT1 | AKT2 |
|---|---|---|---|---|
| | proliferation | E17K IC₅₀: | WT IC₅₀: | IC₅₀: |
| Cmpd. | IC₅₀: GeoMean | GeoMean | GeoMean | GeoMean |
| No. | IC₅₀ (nM) | IC₅₀ (nM) | IC₅₀ (nM) | IC₅₀ (nM) |
| 1 | n.d. | B | C | C |
| 2 | n.d. | C | D | D |
| 3 | n.d. | B | C | D |
| 4 | n.d. | B | C | C |
| 5 | B | B | B | B |
| 6 | n.d. | B | B | C |
| 7 | n.d. | C | D | D |
| 8 | B | B | B | C |
| 9 | n.d. | B | B | C |
| 10 | B | A | B | C |
| 11 | C | B | B | C |
| 12 | D | C | C | C |
| 13 | C | B | C | D |
| 14 | A | A | A | B |
| 15 | C | B | C | D |
| 16 | C | B | B | B |
| 17 | C | C | C | C |
| 18 | C | B | B | C |
| 19 | B | A | A | A |
| 20 | B | B | B | C |
| 21 | B | B | B | B |
| 22 | B | B | B | B |
| 23 | B | B | B | C |
| 24 | B | B | B | C |
| 25 | B | B | B | C |
| 26 | C | B | C | C |
| 27 | C | B | C | C |
| 28 | B | B | B | C |
| 29 | B | A | B | C |
| 30 | D | C | C | C |
| 31 | C | B | B | C |
| 32 | C | B | C | D |
| 33 | B | B | B | C |
| 34 | C | B | B | C |
| 35 | A | A | B | B |
| 36 | C | B | B | C |
| 37 | C | B | C | D |
| 38 | C | B | C | D |
| 39 | C | C | C | C |
| 40 | D | D | C | D |
| 41 | C | C | C | D |
| 42 | C | B | C | C |
| 43 | C | B | C | D |
| 44 | C | C | C | D |
| 45 | C | C | C | D |
| 46 | C | C | C | D |
| 47 | B | B | B | B |
| 48 | C | B | C | D |
| 49 | C | C | C | D |
| 50 | C | B | C | D |
| 51 | B | A | B | B |
| 52 | B | A | A | B |
| 53 | C | A | B | C |
| 54 | D | C | B | C |
| 55 | D | C | C | D |
| 56 | B | A | B | C |
| 57 | B | B | B | C |
| 58 | B | B | B | C |
| 59 | B | B | B | C |
| 60 | n.d | n.d | n.d | n.d |
| 61 | B | B | B | C |
| 62 | C | B | B | C |
| 63 | A | A | A | B |
| 64 | B | B | B | B |
| 65 | n.d | n.d | n.d | n.d |
| 66 | B | A | B | C |
| 67 | C | C | C | C |
| 68 | C | B | B | C |
| 69 | C | B | C | D |
| 70 | C | B | B | C |
| 71 | B | B | B | C |
| 72 | C | B | B | C |
| 73 | B | A | B | C |
| 74 | C | B | C | C |
| 75 | C | B | B | C |
| 76 | C | B | C | C |
| 77 | C | B | B | C |
| 78 | C | B | C | C |
| 79 | D | C | C | D |
| 80 | C | B | B | C |
| 81 | C | B | B | C |
| 82 | B | B | B | B |
| 83 | C | B | B | C |
| 84 | C | B | C | C |
| 85 | D | C | C | C |
| 86 | B | B | B | C |
| 87 | C | B | C | D |
| 88 | B | B | B | C |
| 89 | C | C | B | C |
| 90 | C | B | C | C |
| 91 | B | B | B | C |
| 92 | C | B | B | C |
| 93 | C | B | B | C |
| 94 | C | B | C | C |
| 95 | B | B | B | C |
| 96 | C | B | C | C |
| 97 | C | B | C | C |
| 98 | C | B | B | C |
| 99 | C | C | C | C |
| 100 | C | B | C | C |
| 101 | C | B | B | C |
| 102 | C | B | B | B |
| 103 | B | B | B | C |
| 104 | A | A | B | C |
| 105 | B | B | B | C |
| 106 | B | B | B | C |
| 107 | C | C | C | D |
| 108 | C | B | C | D |
| 109 | C | B | C | C |
| 110 | C | B | B | C |
| 111 | D | C | C | D |
| 112 | D | B | B | C |
| 113 | B | A | B | C |
| 114 | B | B | B | C |
| 115 | B | B | B | C |
| 116 | C | B | B | C |
| 117 | B | B | B | C |
| 118 | B | B | B | C |
| 119 | B | A | B | C |
| 120 | B | A | B | C |
| 121 | B | A | B | C |
| 122 | B | A | B | C |
| 123 | B | A | B | B |
| 124 | B | B | B | C |
| 125 | B | A | B | C |
| 126 | B | A | B | C |
| 127 | B | B | B | C |
| 128 | n.d. | n.d. | n.d. | n.d. |
| 129 | C | B | B | C |
| 130 | B | B | B | C |
| 131 | B | B | B | B |
| 132 | B | A | B | C |
| 133 | B | A | A | B |
| 134 | C | B | C | C |
| 135 | B | B | B | C |
| 136 | C | B | C | D |
| 137 | B | A | A | A |
| 138 | B | B | C | C |
| 139 | n.d. | n.d. | n.d. | n.d. |
| 140 | A | A | A | B |
| 141 | C | C | B | C |
| 142 | C | B | A | B |
| 143 | C | C | C | C |
| 144 | B | B | B | B |
| 145 | C | B | B | C |
| 146 | C | B | C | C |
| 147 | B | B | B | C |
| 148 | C | B | C | C |
| 149 | C | B | C | C |
| 150 | B | B | B | C |
| 151 | B | A | B | C |
| 152 | C | B | C | C |
| 153 | B | B | B | B |
| 154 | B | A | B | C |
| 155 | D | B | B | B |
| 156 | A | B | B | B |
| 157 | B | A | B | B |
| 158 | B | B | B | C |
| 159 | C | B | B | C |
| 160 | D | C | C | C |
| 161 | C | B | B | C |
| 162 | B | A | A | A |
| 163 | D | B | B | D |
| 165 | C | B | B | C |
| 166 | D | D | D | D |
| 167 | D | C | C | C |
| 168 | C | B | B | C |
| 169 | C | D | D | D |
| 170 | B | A | A | C |
| 171 | C | B | B | C |
| 172 | B | B | B | C |
| 173 | C | B | B | C |
| 174 | C | B | B | C |
| 175 | A | A | A | B |
| 176 | C | B | C | C |
| 177 | D | D | D | C |
| 178 | B | B | B | C |
| 179 | C | B | B | C |
| 180 | B | B | B | B |
| 181 | C | B | B | C |
| 182 | C | B | B | C |
| 183 | C | B | B | C |
| 184 | B | B | B | C |
| 185 | B | B | B | C |
| 186 | B | B | B | C |
| 188 | n.d. | B | B | C |
| 189 | D | C | C | D |
| 190 | C | B | C | C |
| 191 | n.d. | B | B | B |
| 192 | D | C | C | D |
| 193 | B | B | B | B |
| 194 | C | B | C | C |
| 195 | B | B | C | C |
| 196 | C | B | B | C |
| 197 | C | B | B | B |
| 198 | C | B | B | C |
| 199 | C | A | A | C |
| 200 | C | B | B | C |
| 201 | C | B | C | C |
| 202 | B | B | B | B |
| 203 | C | B | B | C |
| 204 | B | A | A | B |
| 205 | C | B | B | C |
| 206 | B | A | B | C |
| 207 | C | B | B | C |
| 208 | D | D | D | D |
| 209 | B | B | B | C |
| 210 | C | B | B | C |
| 211 | B | A | B | C |
| 212 | B | A | A | B |
| 213 | C | B | B | C |
| 214 | B | A | B | C |
| 215 | C | B | C | C |
| 216 | C | B | C | C |
| 217 | D | D | D | D |
| 218 | C | C | C | C |
| 219 | D | D | C | C |
| 220 | C | C | C | C |
| 221 | B | B | B | C |
| 222 | B | B | B | B |
| 223 | C | B | C | C |
| 224 | B | B | B | C |
| 225 | B | B | B | C |
| 226 | B | B | B | C |
| 227 | B | B | B | C |
| 228 | C | B | C | D |
| 229 | C | B | C | C |
| 230 | C | B | B | C |
| 231 | C | B | B | C |
| 232 | D | D | D | D |
| 233 | C | C | C | C |
| 234 | B | B | B | C |
| 235 | C | B | C | C |
| 236 | C | B | A | B |
| 237 | C | B | B | C |
| 238 | D | C | D | D |
| 239 | D | B | B | C |
| 240 | B | B | B | C |
| 241 | B | A | B | C |
| 242 | C | B | C | D |
| 243 | C | n.d. | n.d. | n.d. |
| 244 | C | n.d. | n.d. | n.d. |
| 245 | B | A | B | C |
| 246 | B | B | B | C |
| 247 | B | B | B | C |
| 248 | C | B | B | C |
| 249 | D | n.d. | n.d. | n.d. |
| 250 | D | D | D | D |
| 251 | n.d. | B | C | C |
| 252 | C | C | C | D |
| 253 | C | C | C | C |
| 254 | n.d. | n.d. | n.d. | n.d. |
| 255 | C | B | C | C |
| 256 | B | B | B | C |
| 257 | C | B | B | C |
| 258 | B | A | B | B |
| 259 | B | B | B | C |

1. A compound represented by the structure of Formula (A): 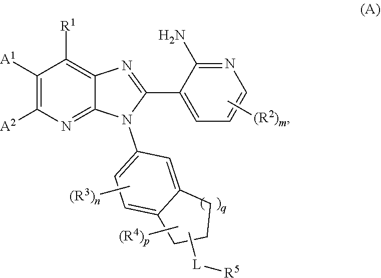
or a pharmaceutically acceptable salt thereof;
wherein:
R¹ is selected from:
hydrogen, halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN;
C₁₋₆ alkyl, C₂₋₆ alkenyl, and C₂₋₆ alkynyl, any of which is optionally substituted with one or more substituents independently selected from: halogen —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, —NO₂, and —CN; and
3- to 6-membered heterocycle and C₃₋₆ carbocycle, any of which is optionally substituted with one or more substituents independently selected from: halogen, —OR¹⁰, —SR¹⁰, —N(R¹⁰)₂, —C(O)N(R¹⁰)₂, —N(R¹⁰)C(O)R¹⁰, —C(O)OR¹⁰, —OC(O)R¹⁰, —N(R¹⁰)C(O)OR¹⁰, —OC(O)N(R¹⁰)₂, —N(R¹⁰)C(O)N(R¹⁰)₂, —S(O)R¹⁰, —S(O)₂R¹⁰, —N(R¹⁰)S(O)₂R¹⁰, —S(O)₂N(R¹⁰)₂, ═O, —NO₂, —CN, C₁₋₆ alkyl, and C₁₋₆ haloalkyl;
A¹ and A² are each independently selected from:
hydrogen, halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN;
C₁₋₆ alkyl, C₂₋₆ alkenyl, and C₂₋₆ alkynyl, any of which is optionally substituted with one or more substituents independently selected from: halogen, —OR¹¹, —SR¹¹, —N(R¹¹)₂, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
5- to 10-membered heterocycle and C₃₋₁₀ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₂₋₆ alkynyl, any one of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₃₋₁₀ carbocycle and 3- to 10-membered heterocycle; wherein the C₃₋₁₀ carbocycle and 3- to 10-membered heterocycle are each optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), and —CN;
C₁₋₆ alkyl, C₂₋₆ alkenyl, and C₂₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —NO₂, ═O, ═S, ═N(R¹¹), and —CN;
q is selected from 1, 2, and 3;
m and n are each independently selected from 0, 1, 2, and 3;
R² is independently selected at each instance from halogen, C₁₋₄ alkyl, C₁₋₄ haloalkyl, —OR¹², —SR¹², —N(R¹²)₂, —NO₂, and —CN;
R³ is independently selected at each instance from:
halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, and —CN; and
C₁₋₆ alkyl optionally substituted with one or more substituents independently selected from: halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —NO₂, ═O, ═S, ═N(R¹³), and —CN;
p is selected from 0, 1, 2, 3, 4, and 5;
R⁴ is independently selected at each instance from:
halogen, —OR⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, and —CN; and
C₁₋₆ alkyl, C₂₋₆ alkenyl, C₂₋₆ alkynyl, any one of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —NO₂, ═O, ═S, ═N(R¹⁴), and —CN; or
two R⁴ attached to the same atom are taken together to form a group selected from:
═O, ═S, and ═N(R¹⁴); or
two R⁴ attached to the same atom or to adjacent atoms are taken together with the carbons to which they are attached to form a group selected from 3- to 8-membered heterocycle and C₃₋₈ carbocycle, any of which is optionally substituted with one or more substituents independently selected from: halogen, C₁₋₄ alkyl, C₁₋₄ haloalkyl, —OR¹⁴, —SR¹⁴, —N(R¹⁴)₂, —NO₂, and —CN;
L is a bond or represented by -L¹-L²-L³-L⁴-, wherein L¹, L², L³, and L⁴ are each independently selected from (a) and (b):
(a) —O—, —N(R¹⁵)—, —S—, —S(O)—, —S(O)₂—, —S(O)(NR¹⁵)—, —N(R¹⁵)C(O)—, —N(R¹⁵)C(O)O—, —N(R¹⁵)S(O)₂—, —N(R¹⁵)S(O)₂N(R¹⁵)—, —S(O)(NR¹⁵)N(R¹⁵)—, —N(R¹⁵)N(R¹⁵)—, —(R¹⁵)NC(O)N(R¹⁵)—, and —(R¹⁵)NC(O)N(R¹⁵)N(R¹⁵)—; and
(b) C₁₋₆ alkylene, C₂₋₆ alkenylene, C₂₋₆ alkynylene, C₃₋₈ carbocyclene, and 3- to 8-membered heterocyclene, any of which is optionally substituted with one or more substituents independently selected from halogen, —OR¹⁵, —SR¹⁵, ═O, ═S, and —CN;
wherein L², L³, and L⁴ are each optionally absent;
wherein no more than two of L¹, L², L³, and L⁴ are selected from (a) and the two selected are not adjacent;
R⁵ is selected from 3- to 12-membered heterocycle and C₃₋₁₂carbocycle, any of which is substituted by —C(O)H or —C(O)D, wherein R⁵ is further optionally substituted by one or more substituents independently selected from:
halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —N(R¹⁶)S(O)₂N(R¹⁶)₂, —S(O)R¹⁶, —S(O)₂R¹⁶, —NO₂, ═O, ═S, ═N(R¹⁶), —N₃, and —CN;
C₁₋₆ alkyl, C₂₋₆ alkenyl, and C₂₋₆ alkynyl, any of which is optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —N(R¹⁶)S(O)₂N(R¹⁶)₂, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), and —CN; and
4- to 6-membered heterocycle optionally substituted with one or more substituents independently selected from: halogen, —OR¹⁶, —SR¹⁶, —N(R¹⁶)₂, —B(OR¹⁶)₂, —C(O)R¹⁶, —C(O)N(R¹⁶)₂, —C(O)OR¹⁶, —OC(O)R¹⁶, —N(R¹⁶)C(O)R¹⁶, —N(R¹⁶)S(O)₂R¹⁶, —S(O)₂N(R¹⁶)₂, —N(R¹⁶)C(O)N(R¹⁶)₂, —N(R¹⁶)C(O)OR¹⁶, —OC(O)N(R¹⁶)₂, —S(O)R¹⁶, —S(O)₂R¹⁶, —NO₂, ═O, ═S, ═N(R¹⁶), —CN, C₁₋₆ alkyl, and C₁₋₆ haloalkyl;
R¹⁰, R¹¹, R¹², R¹³, R¹⁴, and R¹⁵ are each independently selected at each occurrence from:
hydrogen, C₁₋₄ alkyl, C₃₋₈ carbocycle, 3- to 8-membered heterocycle, and C₁₋₄ haloalkyl;
R¹⁶ is independently selected at each occurrence from:
hydrogen;
C₁₋₄ alkyl optionally substituted with one or more substituents independently selected from:
halogen, —OR²⁰, —SR²⁰, —N(R²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
C₃₋₈ carbocycle and 3- to 8-membered heterocycle, either of which is optionally substituted with one or more substituents independently selected from:
halogen, C₁₋₄ alkyl, C₁₋₄ haloalkyl, —OR²⁰, —SR²⁰, —N(R²⁰)₂, —B(OR²⁰)₂, —C(O)R²⁰, —C(O)OR²⁰, —OC(O)R²⁰, —C(O)N(R²⁰)₂, —N(R²⁰)C(O)R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —N(R²⁰)S(O)₂R²⁰, —S(O)₂N(R²⁰)₂, —NO₂, and —CN; and
C₃₋₈ carbocycle and 3- to 8-membered heterocycle, either of which is optionally substituted with one or more substituents independently selected from:
halogen, C₁₋₄ alkyl, C₁₋₄ haloalkyl, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)R²⁰, —N(R²⁰)S(O)₂R²⁰, —N(R²⁰)C(O)N(R²⁰)₂, —N(R²⁰)C(O)OR²⁰, —OC(O)N(R²⁰)₂, —S(O)R²⁰, —S(O)₂R²⁰, —NO₂, and —CN; and
R²⁰ is independently selected at each occurrence from hydrogen, C₁₋₄ alkyl, C₁₋₄ haloalkyl, C₃₋₈ carbocycle, and 3- to 8-membered heterocycle.

2.-135. (canceled)

136. The compound or salt of claim 1, wherein R¹ is selected from hydrogen, halogen, C₁₋₄ alkyl, C₁₋₄ haloalkyl, —OR¹⁰, and —CN.

137. The compound or salt of claim 1, wherein R² is selected from halogen, C₁₋₄ alkyl, and C₁₋₄ haloalkyl.

138. The compound or salt of claim 1, wherein m is 0.

139. The compound or salt of claim 1, wherein A¹ is selected from hydrogen, halogen, C₁₋₄ alkyl, C₁₋₄ haloalkyl, —OR¹¹, —N(R¹¹)₂ and —CN.

140. The compound or salt of claim 1, wherein A¹ is selected from hydrogen, fluoro, methyl, —OH, and —OCH₃.

141. The compound or salt of claim 1, wherein A² is selected from:
hydrogen, halogen, C₁₋₄ haloalkyl, —OR¹¹, —N(R¹¹)₂, —C(O)N(R¹¹)₂, —N(R¹¹)C(O)R¹¹, and —CN;
C₁₋₆ alkyl, C₂₋₆ alkenyl, and C₂₋₆ alkynyl, any of which is optionally substituted with one or more substituents independently selected from: halogen, —OR¹¹, —N(R¹¹)₂, —C(O)N(R¹¹)₂, —N(R¹¹)C(O)R¹¹, ═O, and —CN; and
5- to 10-membered heterocycle and C₃₋₁₀ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN;
C₁₋₆ alkyl, C₂₋₆ alkenyl, and C₃₋₆ alkynyl, any one of which is optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, —NO₂, ═O, and —CN; and
C₃₋₁₀ carbocycle and 3- to 10-membered heterocycle; wherein the C₃₋₁₀ carbocycle and 3- to 10-membered heterocycle are each optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, ═O, and —CN; and
C₁₋₆ alkyl, C₂₋₆ alkenyl, and C₃₋₆ alkynyl, any one of which is optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, ═O, and —CN.

142. The compound or salt of claim 1, wherein A² is selected from: hydrogen, fluoro, bromo, chloro, methyl, ethyl, ethynylene, trifluoromethyl, 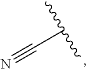 [SMILES: *C#N]
—OCH₃, cyclopropyl,

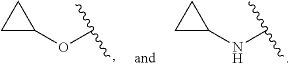 [SMILES: *OC1CC1 ; *NC1CC1]

143. The compound or salt of claim 1, wherein A² is selected from: —F, —Cl, —CH₃, —CN, cyclopropyl, ethynylene, —CF₃, phenyl,

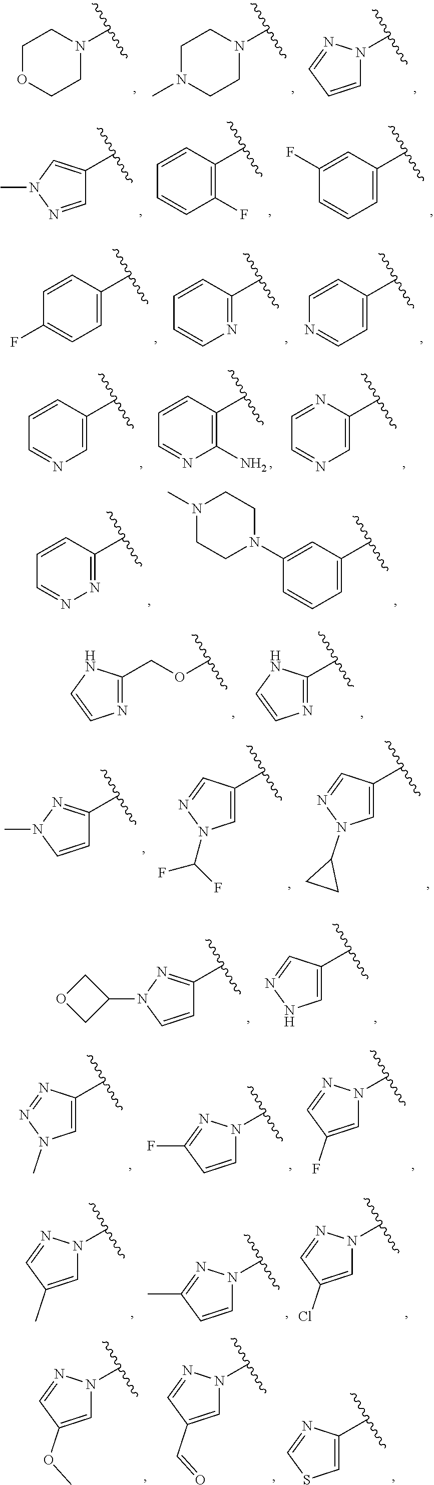

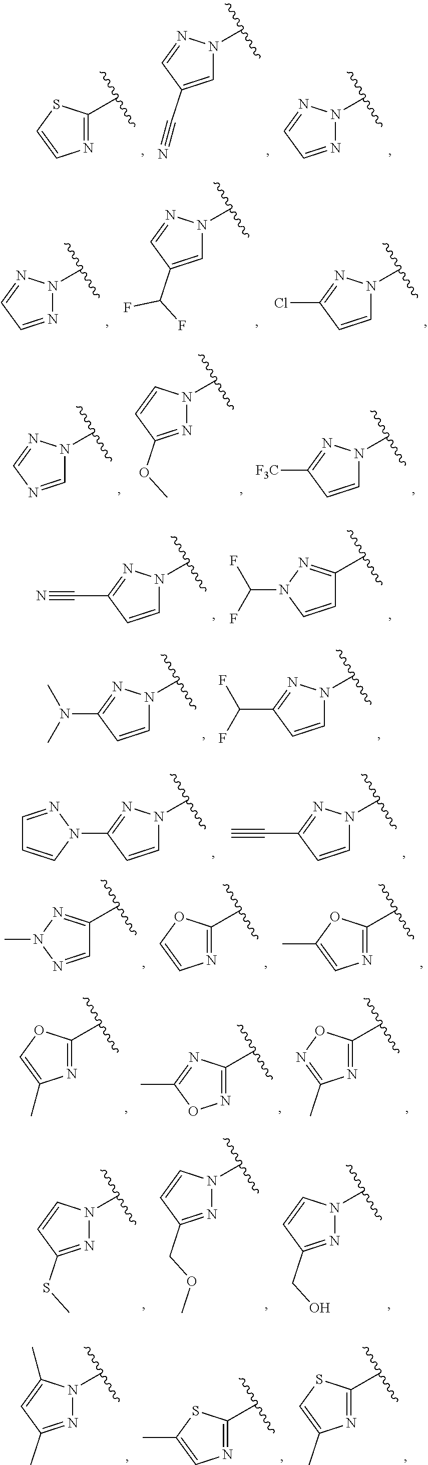

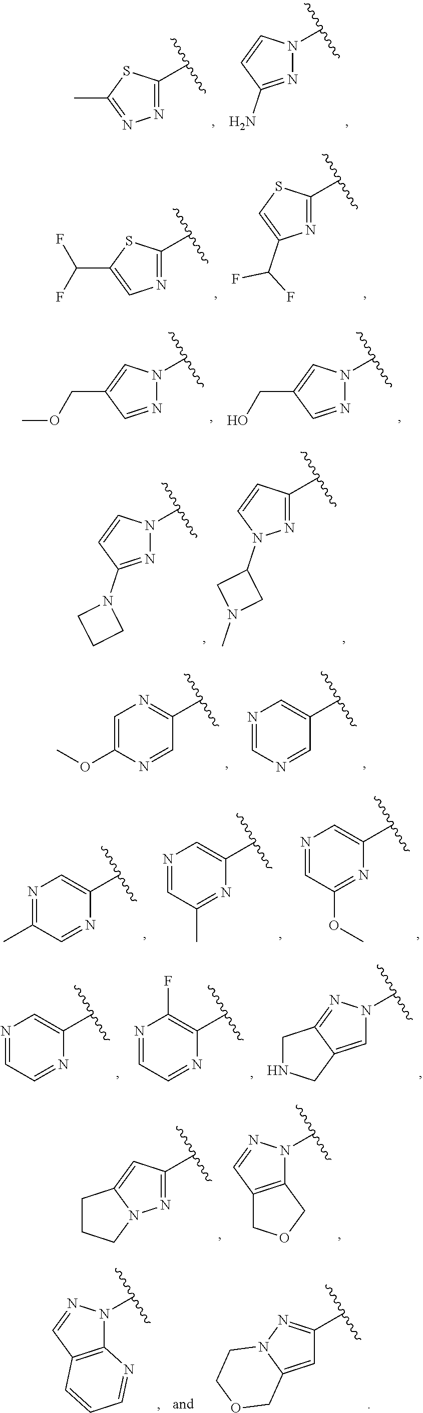

144. The compound or salt of claim 1, wherein R³ is independently selected at each instance from halogen, C₁₋₄ alkyl, C₁₋₄ haloalkyl, and —CN.

145. The compound or salt of claim 1, wherein n is 0.

146. The compound or salt of claim 1, wherein R⁴ is independently selected at each instance from:
halogen, —OR¹⁴, —SR¹⁴, —N(R¹⁴)₂, —CN, C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from: halogen, —OR¹⁴, —N(R¹⁴)₂, ═O, and —CN; or
two R⁴ attached to the same atom are taken together to form ═O; or
two R⁴ attached to the same atom or to adjacent atoms are taken together with the carbons to which they are attached to form a C₃₋₈ carbocycle; and
R¹⁴ is selected from hydrogen and C₁₋₄ alkyl.

147. The compound or salt of claim 1, wherein L¹, L², L³, and L⁴ are each independently selected from (a) and (b):
(a) —O—, —N(R¹⁵)—, —S—, —N(R¹⁵)C(O)—, —N(R¹⁵)C(O)O—, —N(R¹⁵)S(O)₂—N(R¹⁵)N(R¹⁵)—, and —(R¹⁵)NC(O)N(R¹⁵)—; and
(b) C₁₋₆ alkylene, C₂₋₆ alkenylene, C₂₋₆ alkynylene, C₃₋₆ carbocyclene, and 3- to 6-membered heterocyclene, any of which is optionally substituted with one or more substituents independently selected from halogen, —OR¹⁵, ═O, and —CN;
wherein L², L³, and L⁴ are each optionally absent;
wherein no more than two of L¹, L², L³, and L⁴ are selected from (a) and the two selected are not adjacent.

148. The compound or salt of claim 1, wherein L is selected from

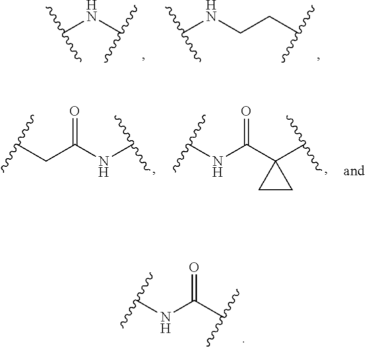

149. The compound or salt of claim 1, wherein R⁵ is selected from 3- to 10-membered heterocycle and C₃₋₁₀ carbocycle, any of which is substituted by —C(O)H; and wherein R⁵ is further optionally substituted by one or more substituents independently selected from: halogen, C₁₋₄ alkyl, C₁₋₄ haloalkyl, —OR¹⁶, —C(O)R¹⁶, —SR¹⁶, —N(R¹⁶)₂, —B(OR¹⁶)₂, —N(R¹⁶)C(O)R¹⁶, —N(R¹⁶)S(O)₂R¹⁶, —S(O)₂N(R¹⁶)₂, N(R¹⁶)C(O)N(R¹⁶)₂, —CN, and 4- to 6-membered heterocycle.

150. The compound or salt of claim 1, wherein R⁵ is selected from:

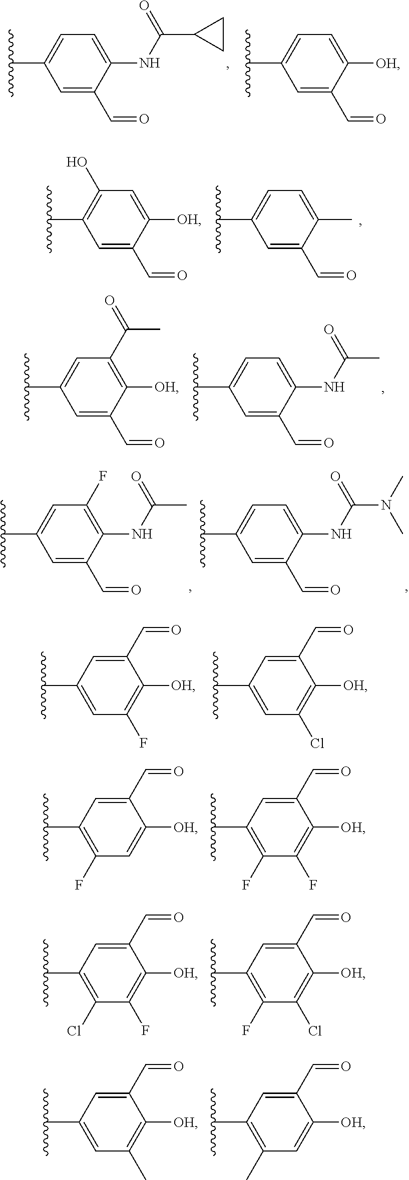

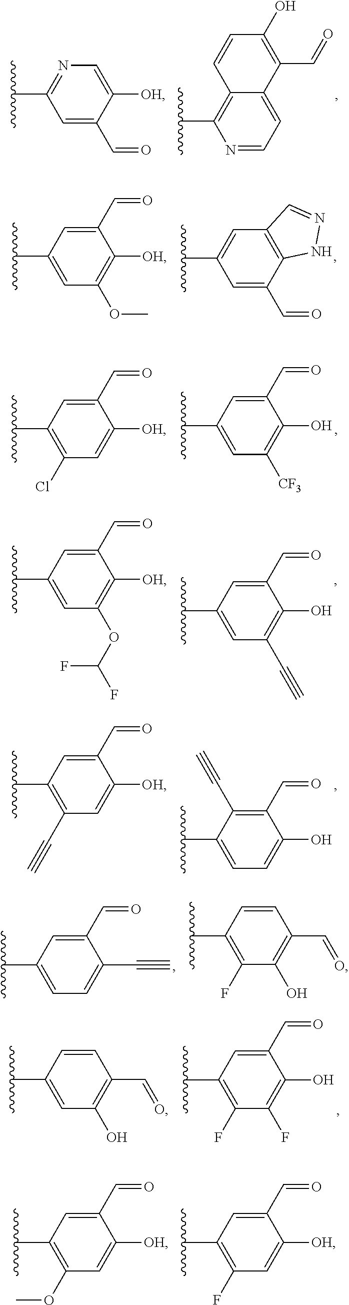

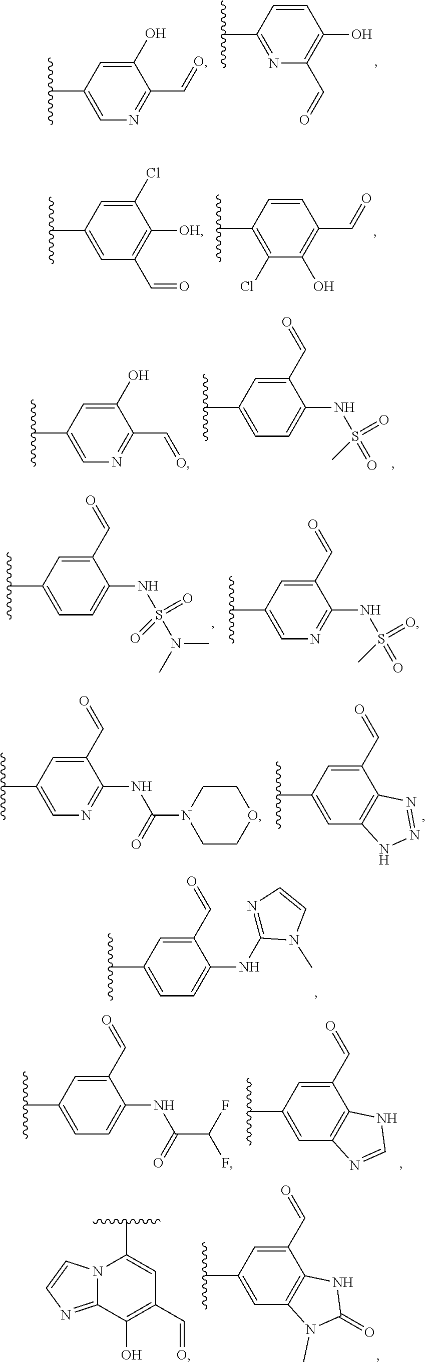

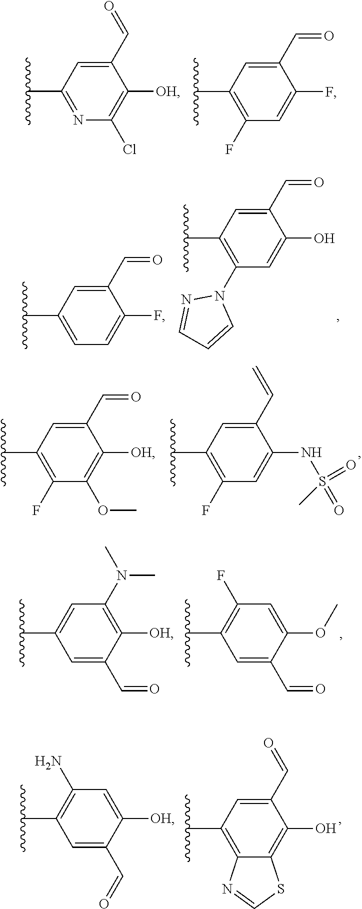

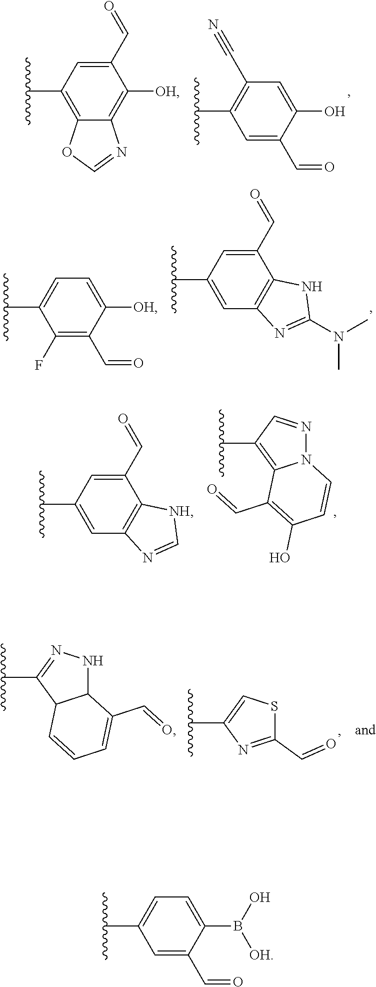

151. The compound or salt of claim 1, where the compound is selected from: 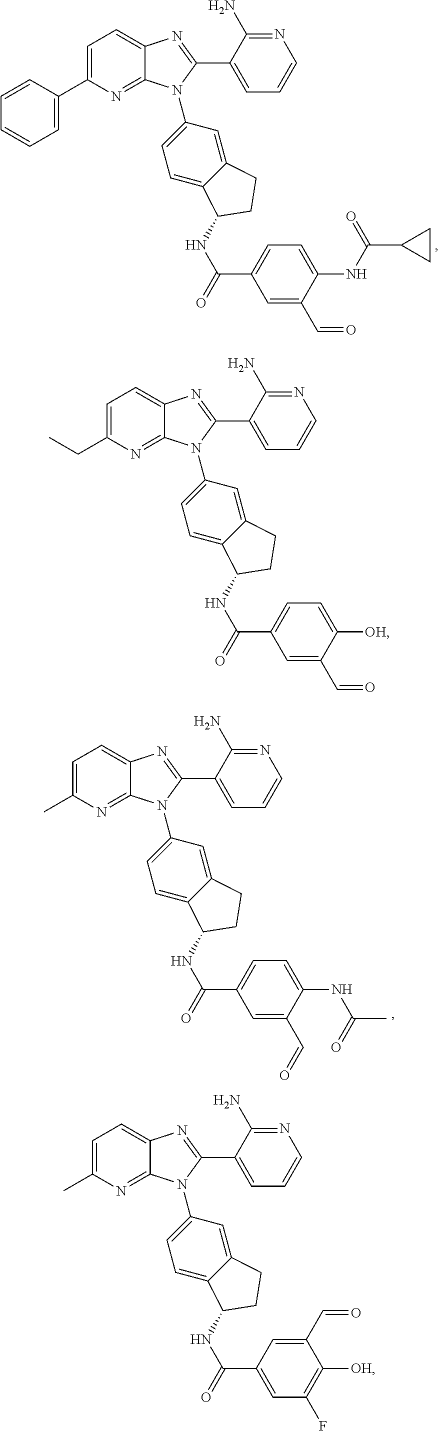 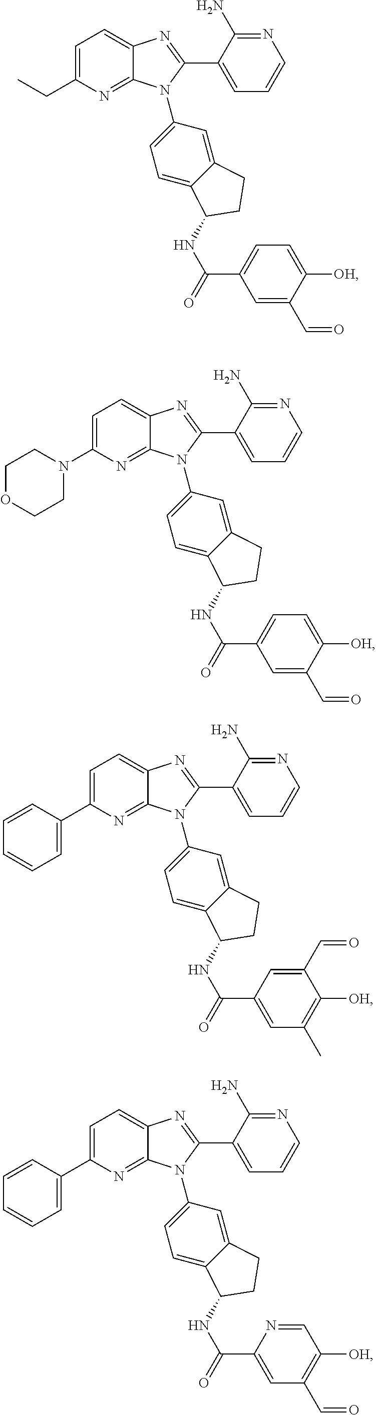 [SMILES: CCc1ccc2nc(-c3cccnc3N)n(-c3ccc4c(c3)CC[C@@H]4NC(=O)c3ccc(O)c(C=O)c3)c2n1 ; Nc1ncccc1-c1nc2ccc(N3CCOCC3)nc2n1-c1ccc2c(c1)CC[C@@H]2NC(=O)c1ccc(O)c(C=O)c1 ; Cc1cc(C(=O)N[C@H]2CCc3cc(-n4c(-c5cccnc5N)nc5ccc(-c6ccccc6)nc54)ccc32)cc(C=O)c1O ; Nc1ncccc1-c1nc2ccc(-c3ccccc3)nc2n1-c1ccc2c(c1)CC[C@@H]2NC(=O)c1cc(C=O)c(O)cn1] 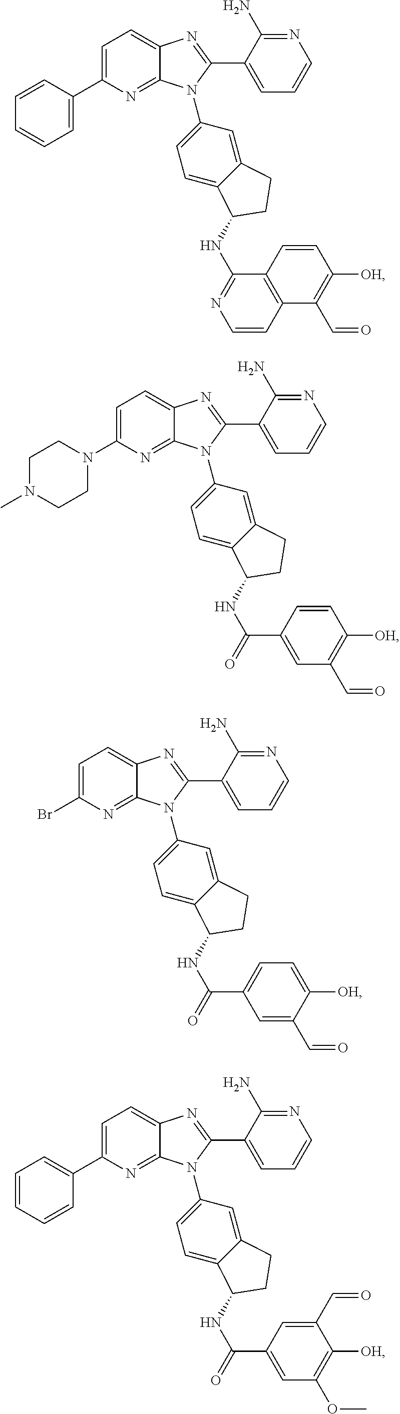 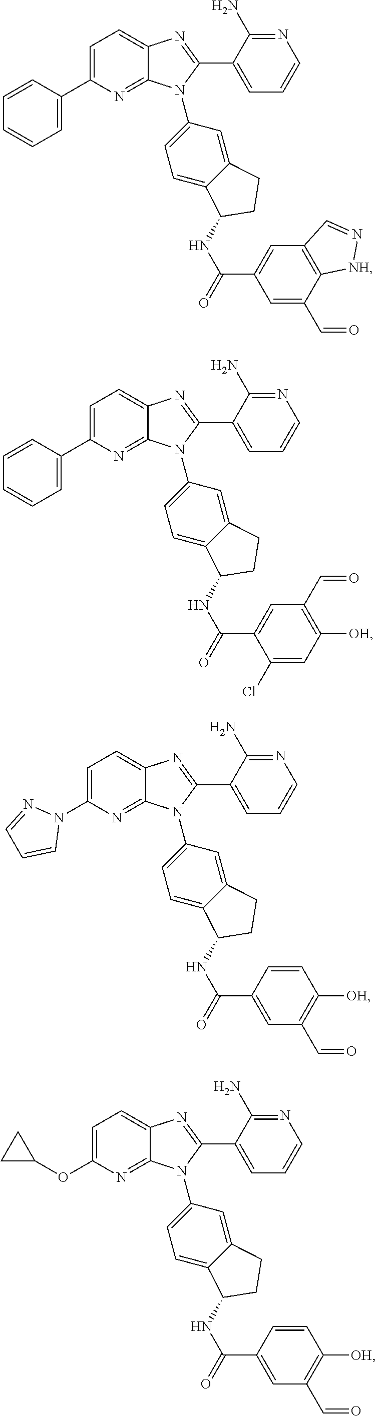 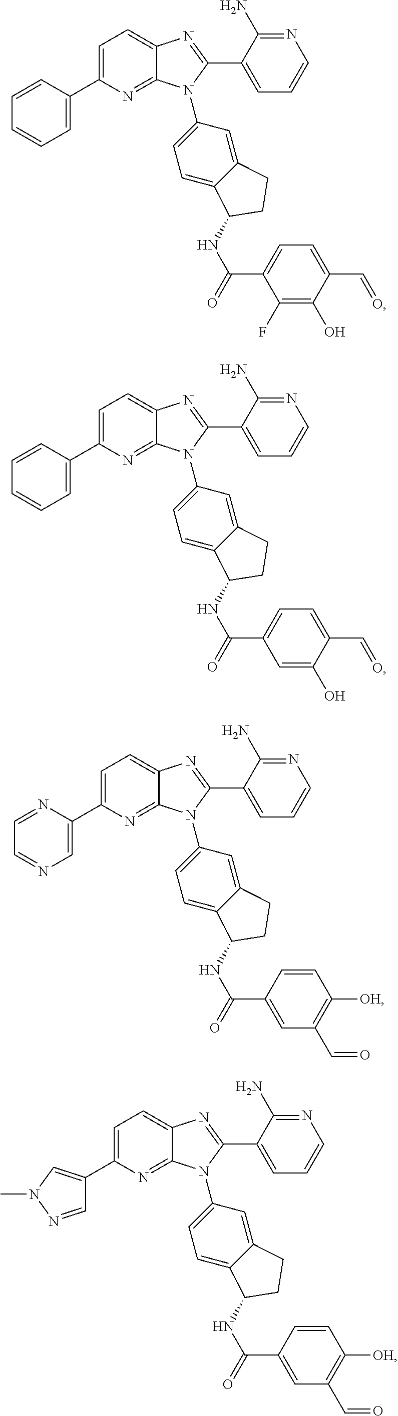 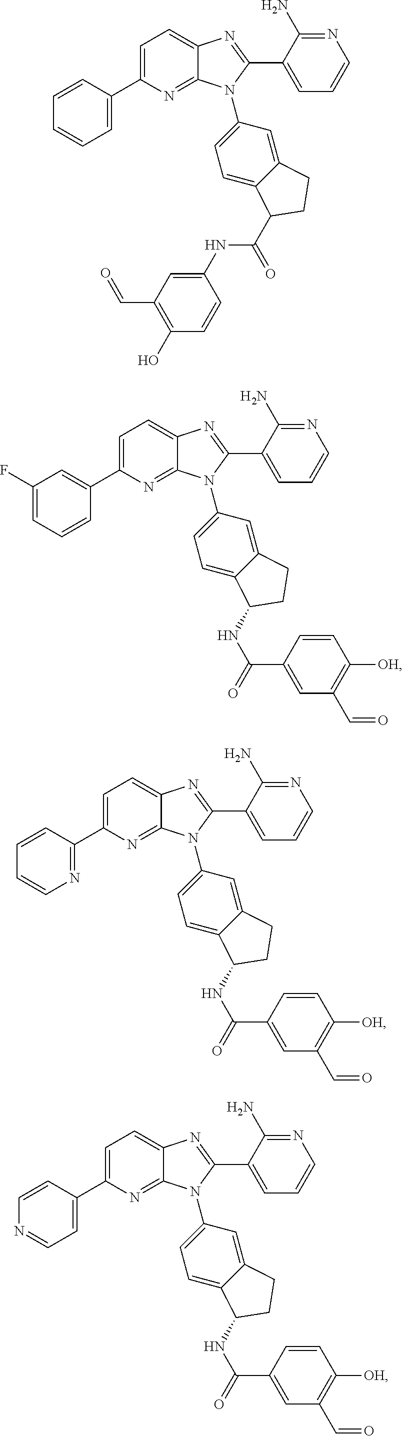 [SMILES: Nc1ncccc1-c1nc2ccc(-c3ccccc3)nc2n1-c1ccc2c(c1)CCC2C(=O)Nc1ccc(O)c(C=O)c1 ; Nc1ncccc1-c1nc2ccc(-c3cccc(F)c3)nc2n1-c1ccc2c(c1)CC[C@@H]2NC(=O)c1ccc(O)c(C=O)c1 ; Nc1ncccc1-c1nc2ccc(-c3ccccn3)nc2n1-c1ccc2c(c1)CC[C@@H]2NC(=O)c1ccc(O)c(C=O)c1 ; Nc1ncccc1-c1nc2ccc(-c3ccncc3)nc2n1-c1ccc2c(c1)CC[C@@H]2NC(=O)c1ccc(O)c(C=O)c1] 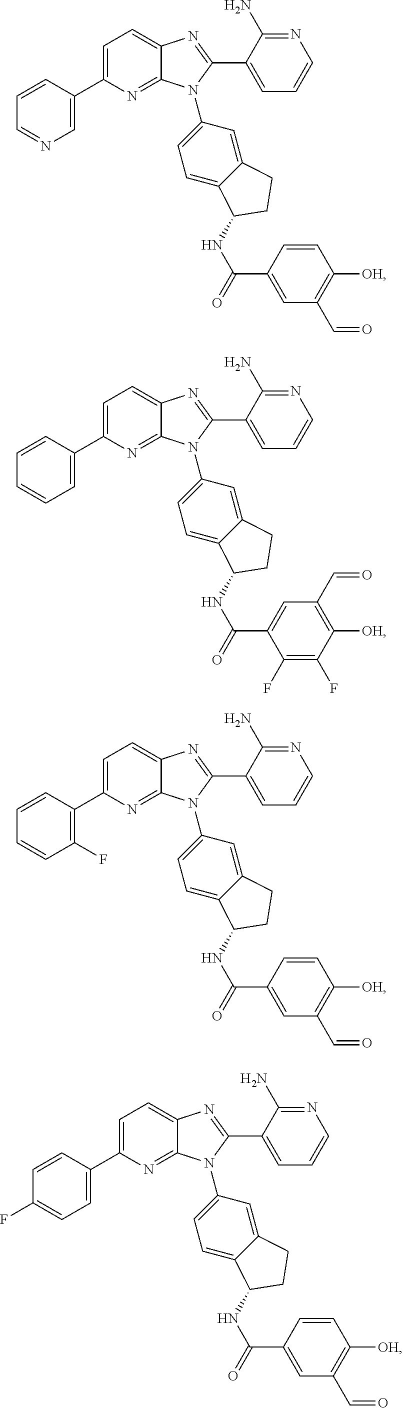 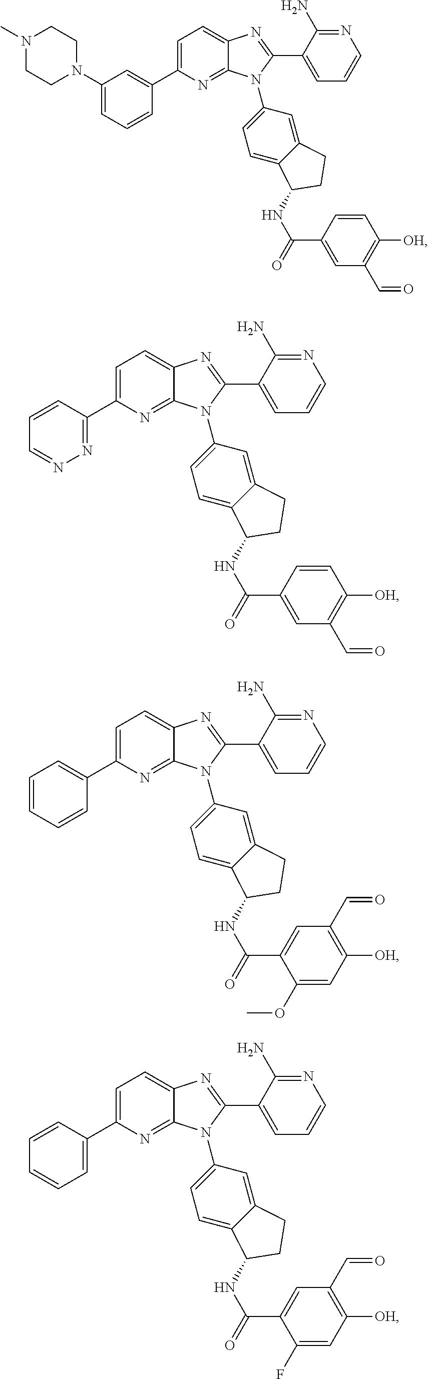 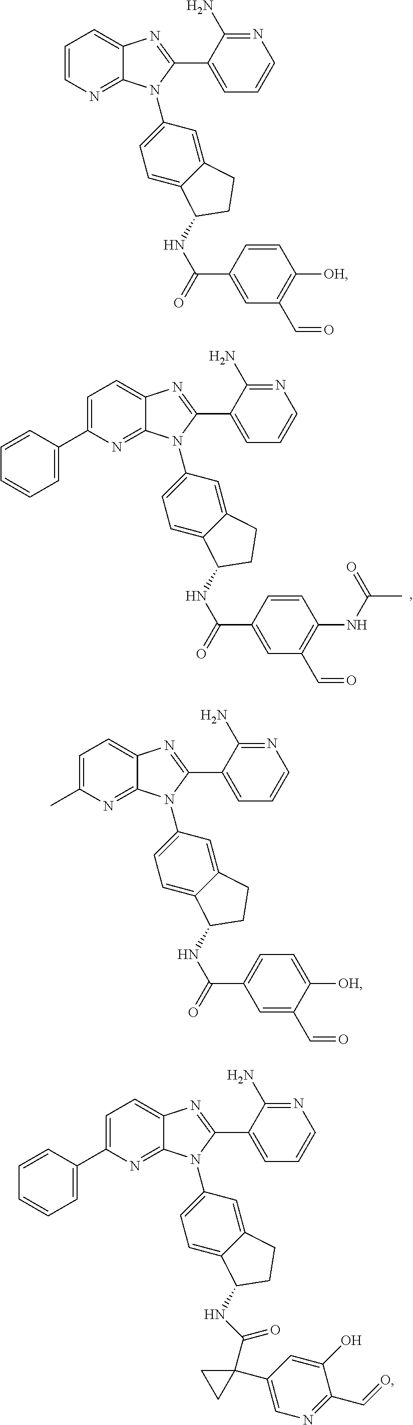 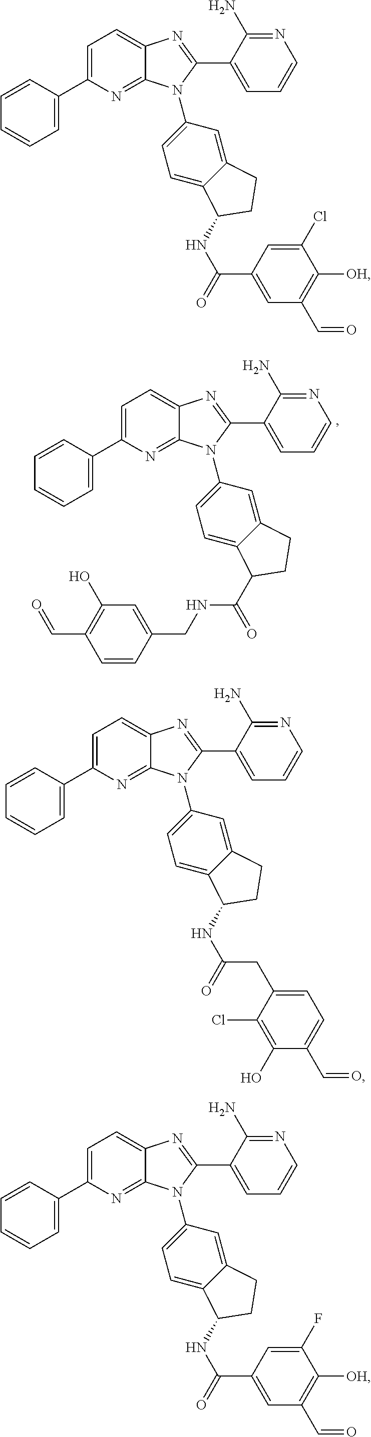 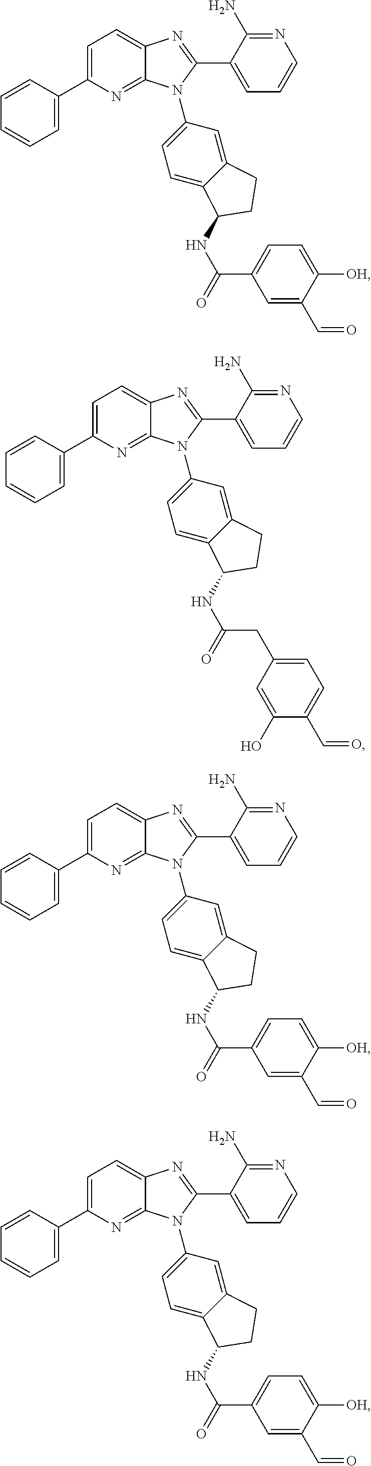 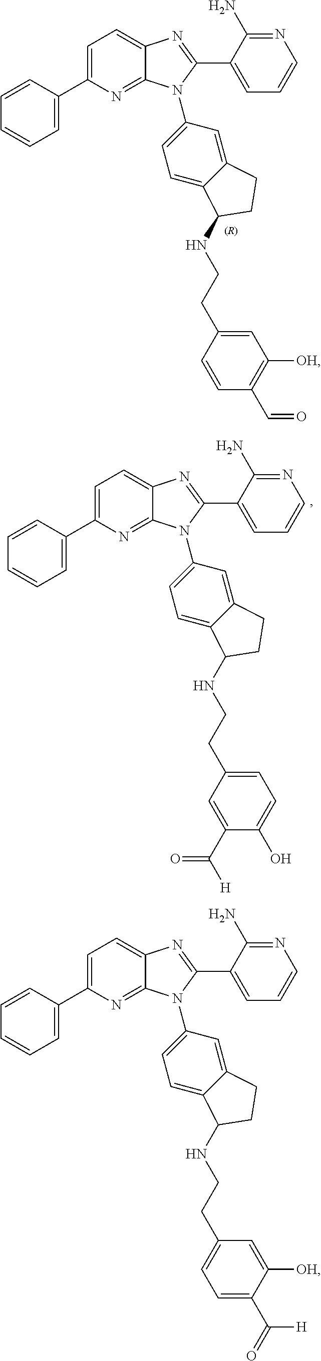 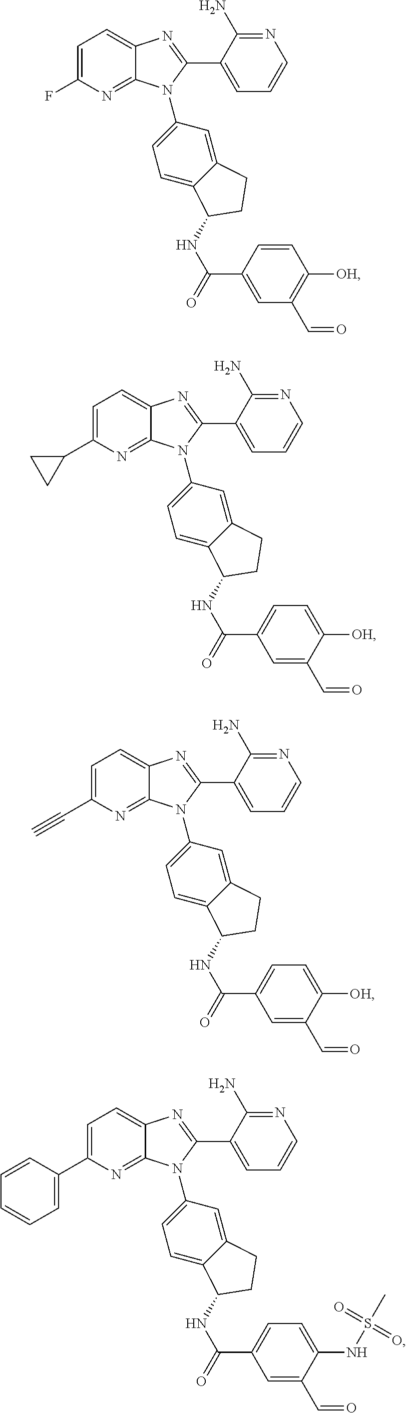 [SMILES: Nc1ncccc1-c1nc2ccc(F)nc2n1-c1ccc2c(c1)CC[C@@H]2NC(=O)c1ccc(O)c(C=O)c1 ; Nc1ncccc1-c1nc2ccc(C3CC3)nc2n1-c1ccc2c(c1)CC[C@@H]2NC(=O)c1ccc(O)c(C=O)c1 ; C#Cc1ccc2nc(-c3cccnc3N)n(-c3ccc4c(c3)CC[C@@H]4NC(=O)c3ccc(O)c(C=O)c3)c2n1 ; CS(=O)(=O)Nc1ccc(C(=O)N[C@H]2CCc3cc(-n4c(-c5cccnc5N)nc5ccc(-c6ccccc6)nc54)ccc32)cc1C=O] 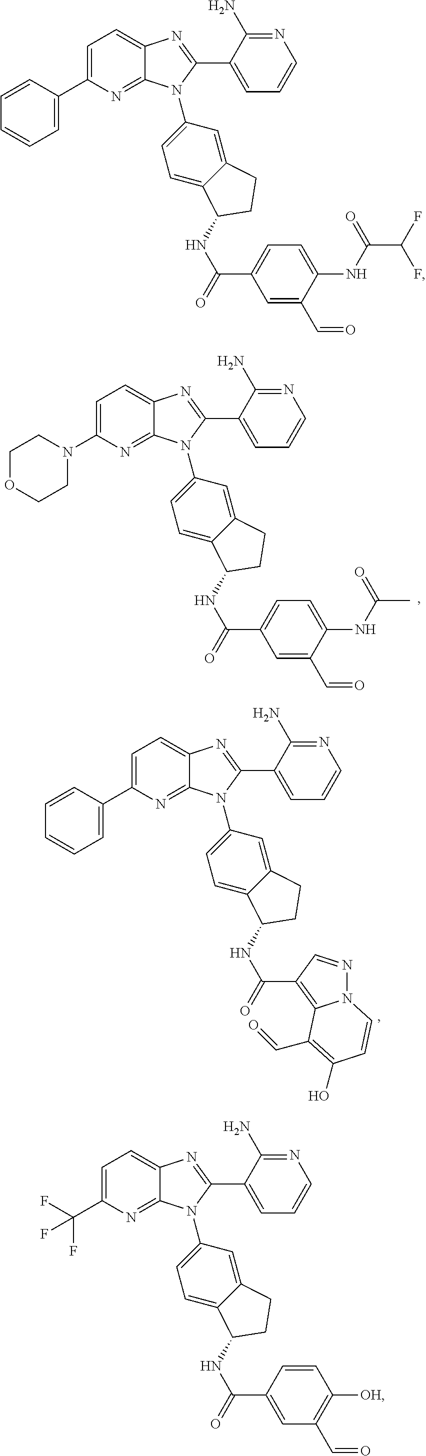 [SMILES: Nc1ncccc1-c1nc2ccc(-c3ccccc3)nc2n1-c1ccc2c(c1)CC[C@@H]2NC(=O)c1ccc(NC(=O)C(F)F)c(C=O)c1 ; CC(=O)Nc1ccc(C(=O)N[C@H]2CCc3cc(-n4c(-c5cccnc5N)nc5ccc(N6CCOCC6)nc54)ccc32)cc1C=O ; Nc1ncccc1-c1nc2ccc(-c3ccccc3)nc2n1-c1ccc2c(c1)CC[C@@H]2NC(=O)c1cnn2ccc(O)c(C=O)c12 ; Nc1ncccc1-c1nc2ccc(C(F)(F)F)nc2n1-c1ccc2c(c1)CC[C@@H]2NC(=O)c1ccc(O)c(C=O)c1] 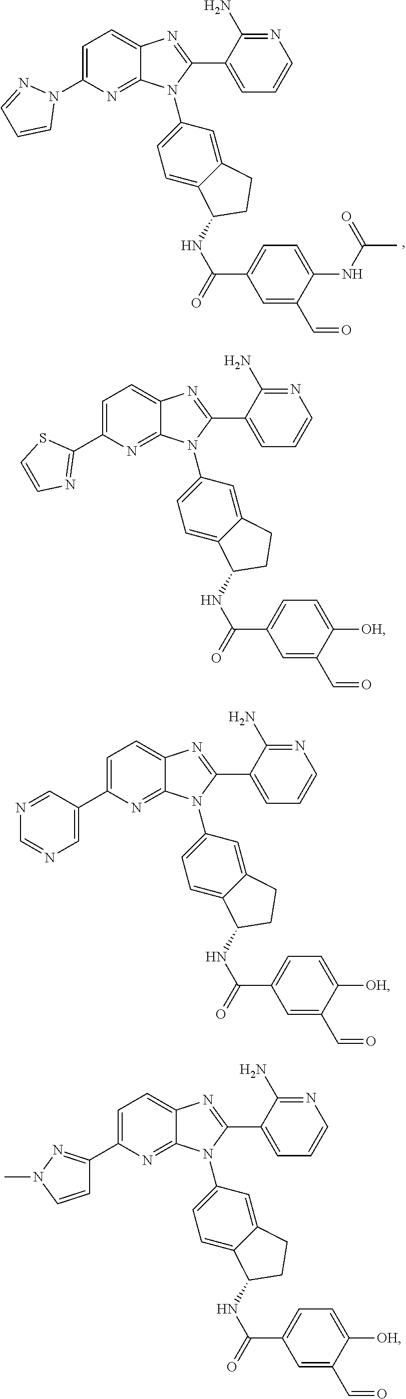 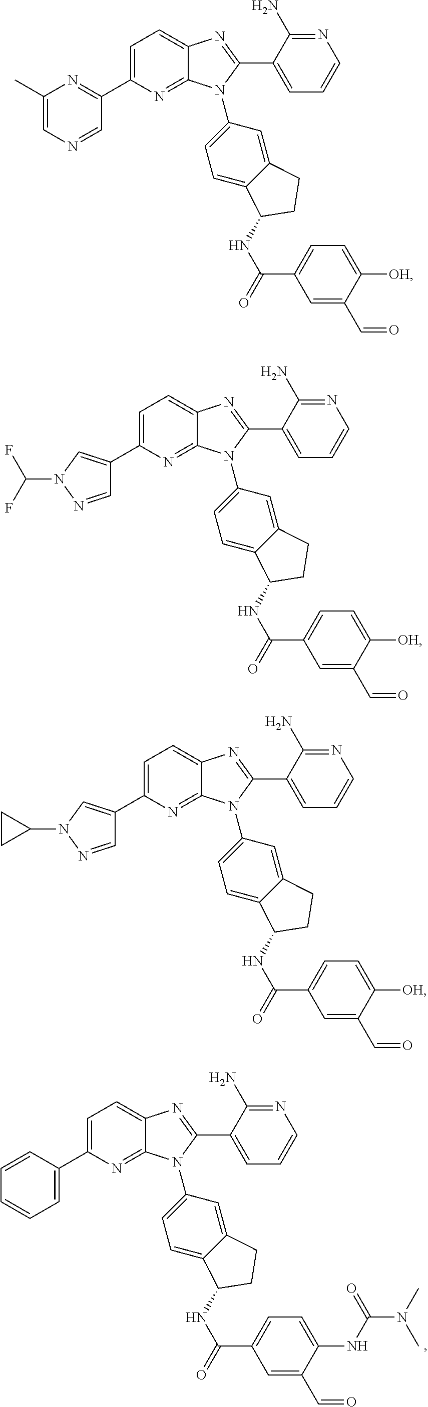 [SMILES: Cc1cncc(-c2ccc3nc(-c4cccnc4N)n(-c4ccc5c(c4)CC[C@@H]5NC(=O)c4ccc(O)c(C=O)c4)c3n2)n1 ; Nc1ncccc1-c1nc2ccc(-c3cnn(C(F)F)c3)nc2n1-c1ccc2c(c1)CC[C@@H]2NC(=O)c1ccc(O)c(C=O)c1 ; Nc1ncccc1-c1nc2ccc(-c3cnn(C4CC4)c3)nc2n1-c1ccc2c(c1)CC[C@@H]2NC(=O)c1ccc(O)c(C=O)c1 ; CN(C)C(=O)Nc1ccc(C(=O)N[C@H]2CCc3cc(-n4c(-c5cccnc5N)nc5ccc(-c6ccccc6)nc54)ccc32)cc1C=O] 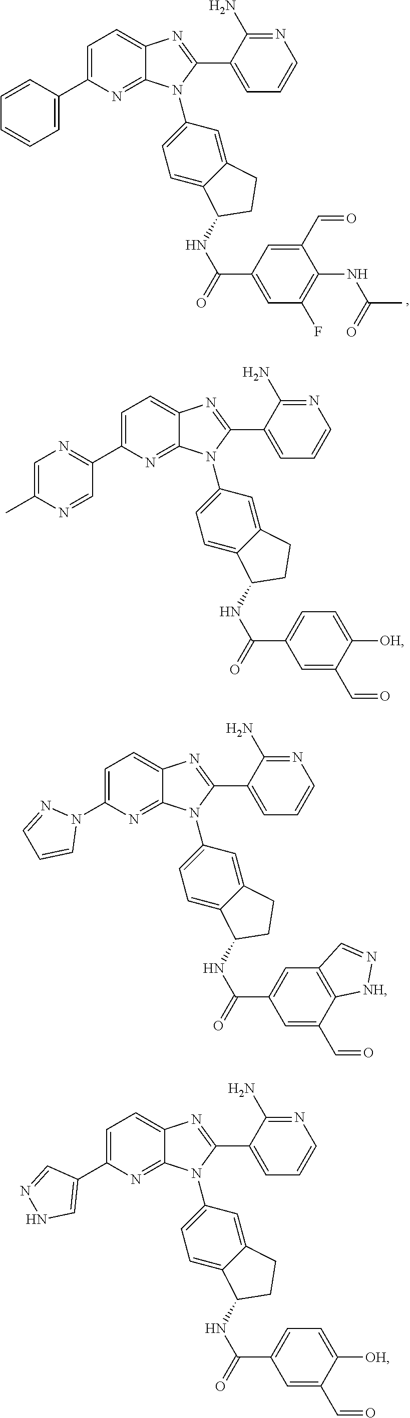 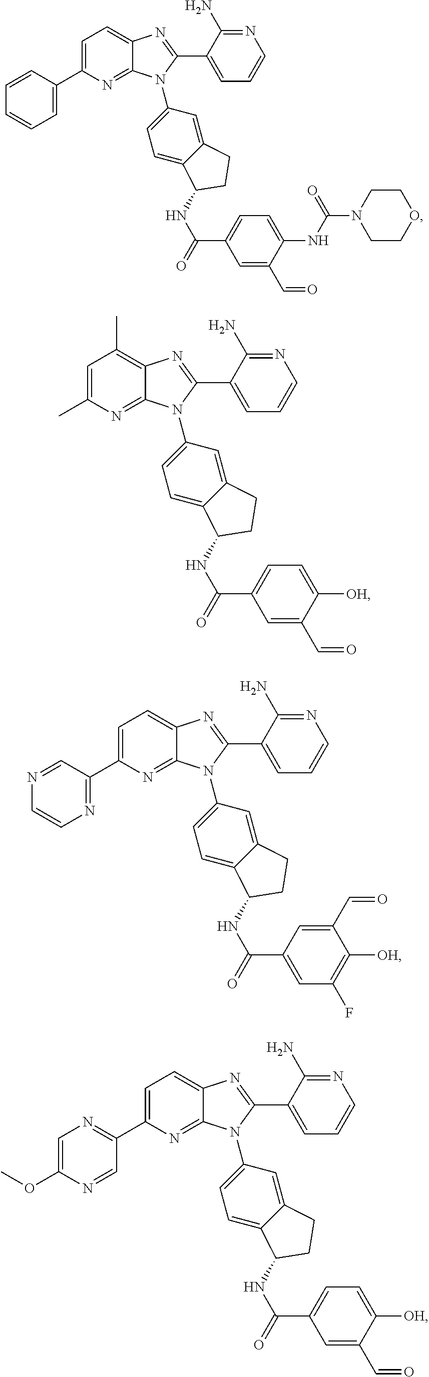 [SMILES: Nc1ncccc1-c1nc2ccc(-c3ccccc3)nc2n1-c1ccc2c(c1)CC[C@@H]2NC(=O)c1ccc(NC(=O)N2CCOCC2)c(C=O)c1 ; Cc1cc(C)c2nc(-c3cccnc3N)n(-c3ccc4c(c3)CC[C@@H]4NC(=O)c3ccc(O)c(C=O)c3)c2n1 ; Nc1ncccc1-c1nc2ccc(-c3cnccn3)nc2n1-c1ccc2c(c1)CC[C@@H]2NC(=O)c1cc(F)c(O)c(C=O)c1 ; COc1cnc(-c2ccc3nc(-c4cccnc4N)n(-c4ccc5c(c4)CC[C@@H]5NC(=O)c4ccc(O)c(C=O)c4)c3n2)cn1] 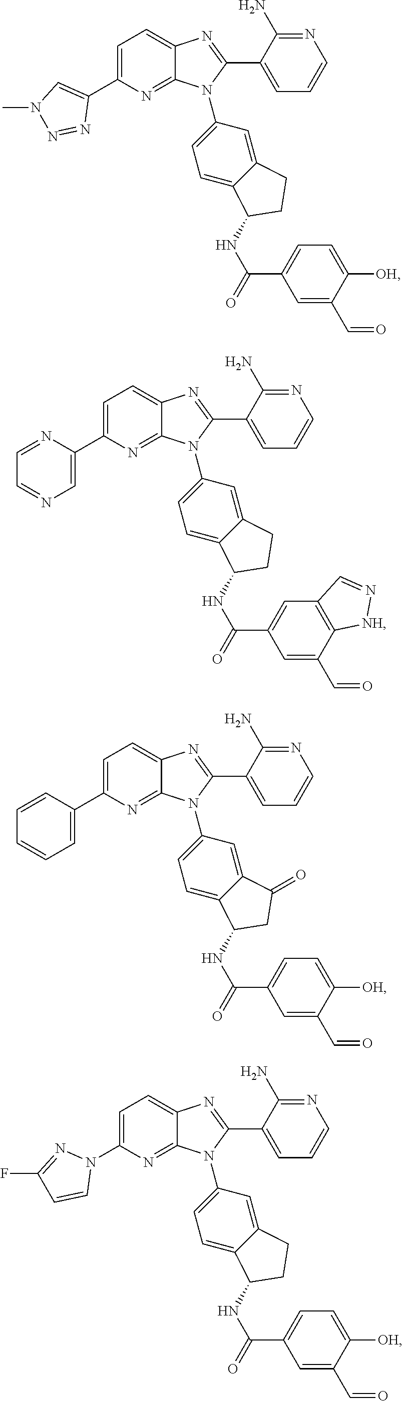 [SMILES: Cn1cc(-c2ccc3nc(-c4cccnc4N)n(-c4ccc5c(c4)CC[C@@H]5NC(=O)c4ccc(O)c(C=O)c4)c3n2)nn1 ; Nc1ncccc1-c1nc2ccc(-c3cnccn3)nc2n1-c1ccc2c(c1)CC[C@@H]2NC(=O)c1cc(C=O)c2[nH]ncc2c1 ; Nc1ncccc1-c1nc2ccc(-c3ccccc3)nc2n1-c1ccc2c(c1)C(=O)C[C@@H]2NC(=O)c1ccc(O)c(C=O)c1 ; Nc1ncccc1-c1nc2ccc(-n3ccc(F)n3)nc2n1-c1ccc2c(c1)CC[C@@H]2NC(=O)c1ccc(O)c(C=O)c1] 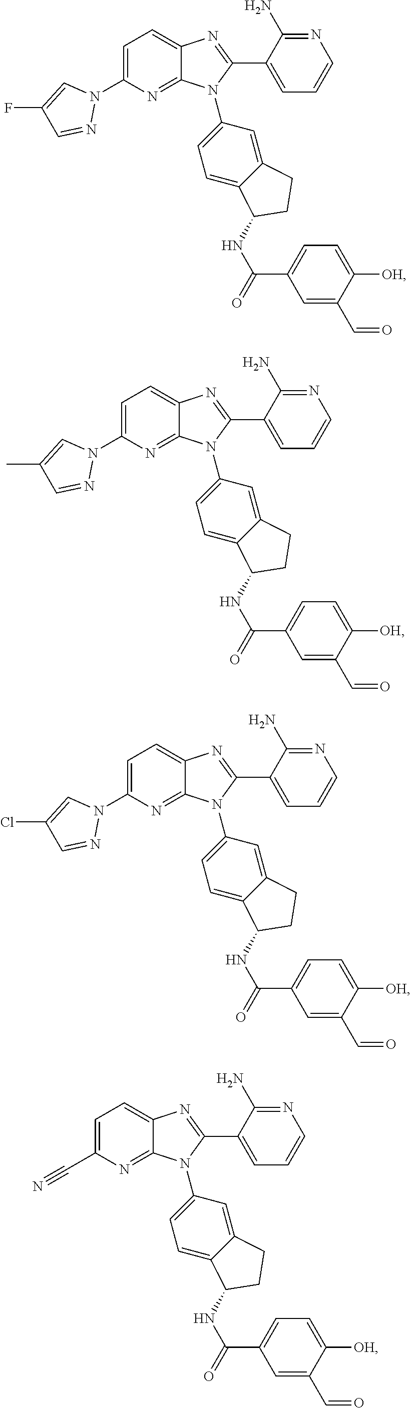 [SMILES: Nc1ncccc1-c1nc2ccc(-n3cc(F)cn3)nc2n1-c1ccc2c(c1)CC[C@@H]2NC(=O)c1ccc(O)c(C=O)c1 ; Cc1cnn(-c2ccc3nc(-c4cccnc4N)n(-c4ccc5c(c4)CC[C@@H]5NC(=O)c4ccc(O)c(C=O)c4)c3n2)c1 ; Nc1ncccc1-c1nc2ccc(-n3cc(Cl)cn3)nc2n1-c1ccc2c(c1)CC[C@@H]2NC(=O)c1ccc(O)c(C=O)c1 ; N#Cc1ccc2nc(-c3cccnc3N)n(-c3ccc4c(c3)CC[C@@H]4NC(=O)c3ccc(O)c(C=O)c3)c2n1] 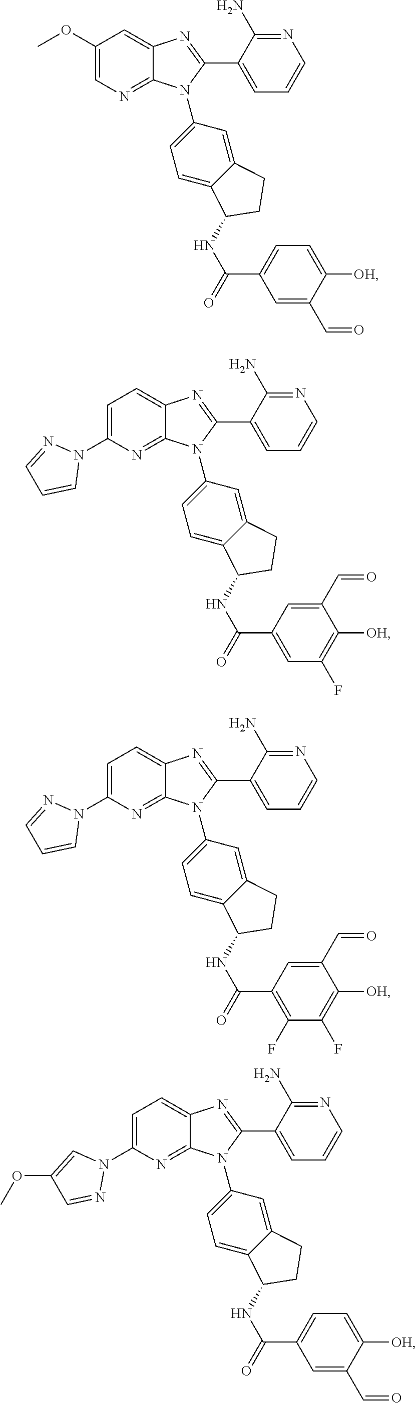 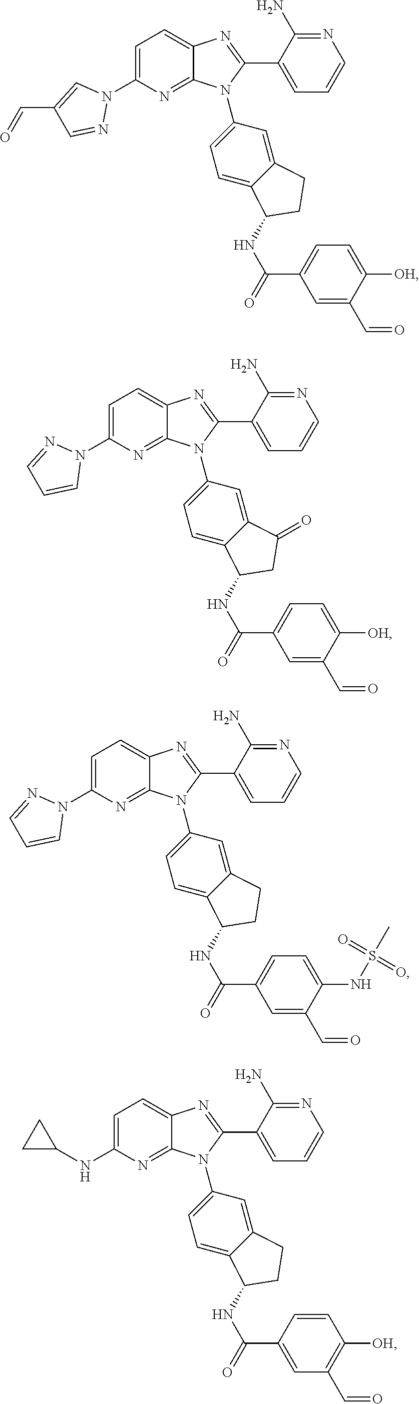 [SMILES: Nc1ncccc1-c1nc2ccc(-n3cc(C=O)cn3)nc2n1-c1ccc2c(c1)CC[C@@H]2NC(=O)c1ccc(O)c(C=O)c1 ; Nc1ncccc1-c1nc2ccc(-n3cccn3)nc2n1-c1ccc2c(c1)C(=O)C[C@@H]2NC(=O)c1ccc(O)c(C=O)c1 ; CS(=O)(=O)Nc1ccc(C(=O)N[C@H]2CCc3cc(-n4c(-c5cccnc5N)nc5ccc(-n6cccn6)nc54)ccc32)cc1C=O ; Nc1ncccc1-c1nc2ccc(NC3CC3)nc2n1-c1ccc2c(c1)CC[C@@H]2NC(=O)c1ccc(O)c(C=O)c1] 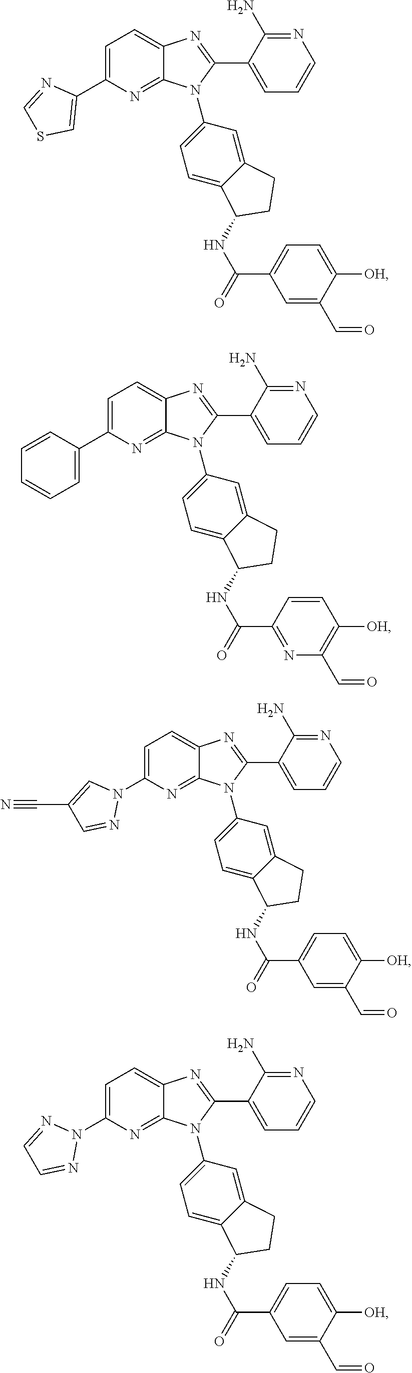 [SMILES: Nc1ncccc1-c1nc2ccc(-c3cscn3)nc2n1-c1ccc2c(c1)CC[C@@H]2NC(=O)c1ccc(O)c(C=O)c1 ; Nc1ncccc1-c1nc2ccc(-c3ccccc3)nc2n1-c1ccc2c(c1)CC[C@@H]2NC(=O)c1ccc(O)c(C=O)n1 ; N#Cc1cnn(-c2ccc3nc(-c4cccnc4N)n(-c4ccc5c(c4)CC[C@@H]5NC(=O)c4ccc(O)c(C=O)c4)c3n2)c1 ; Nc1ncccc1-c1nc2ccc(-n3nccn3)nc2n1-c1ccc2c(c1)CC[C@@H]2NC(=O)c1ccc(O)c(C=O)c1] 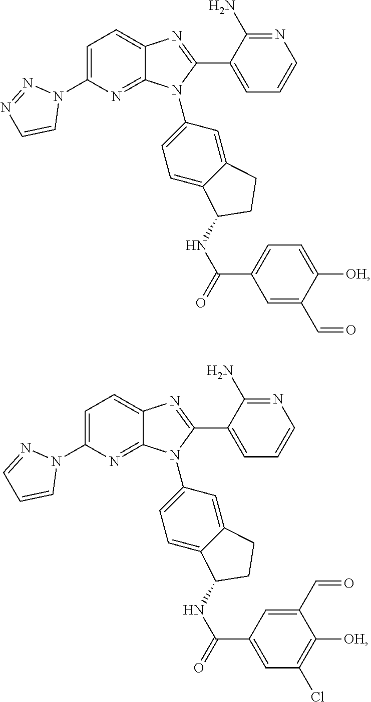 [SMILES: Nc1ncccc1-c1nc2ccc(-n3ccnn3)nc2n1-c1ccc2c(c1)CC[C@@H]2NC(=O)c1ccc(O)c(C=O)c1 ; Nc1ncccc1-c1nc2ccc(-n3cccn3)nc2n1-c1ccc2c(c1)CC[C@@H]2NC(=O)c1cc(Cl)c(O)c(C=O)c1] 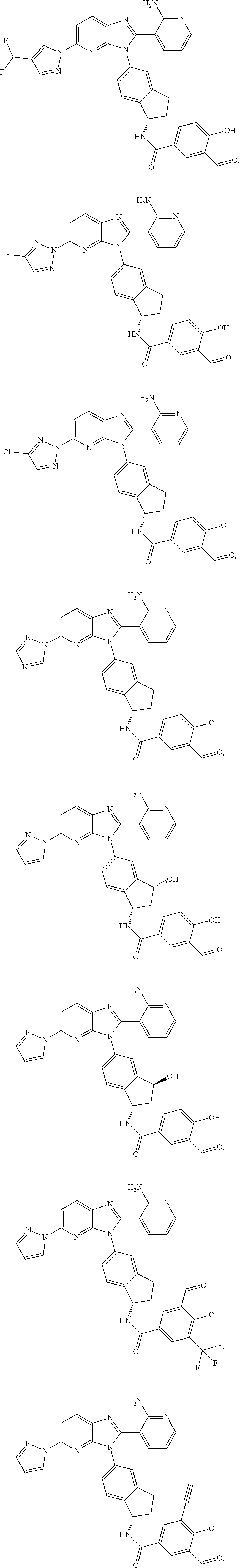 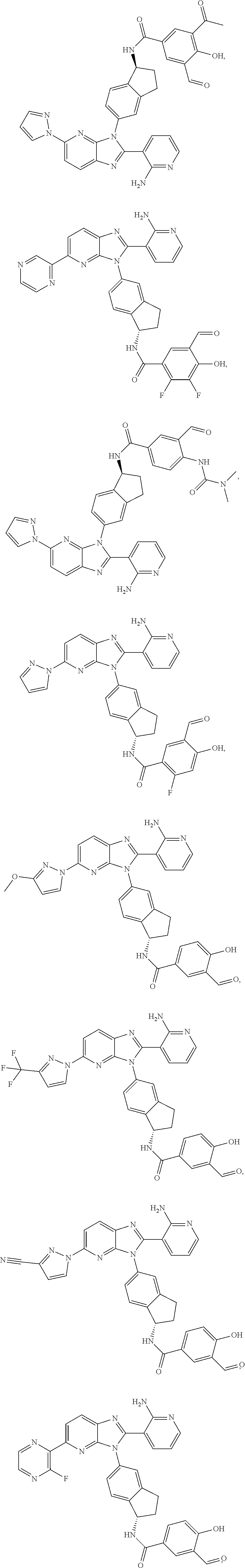 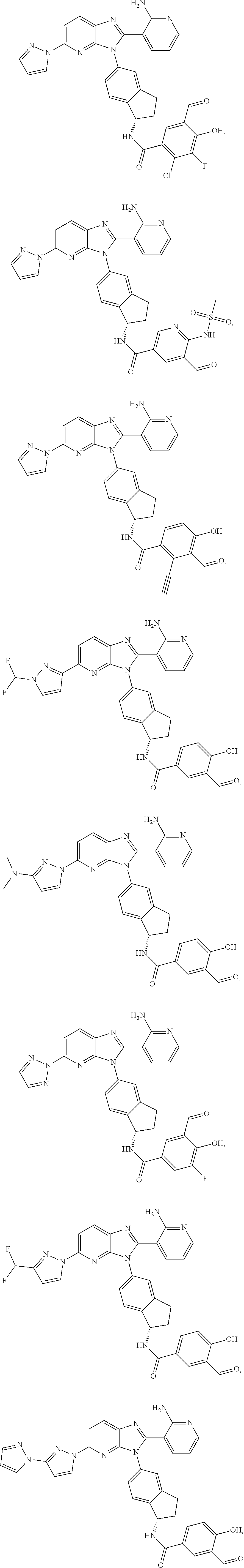 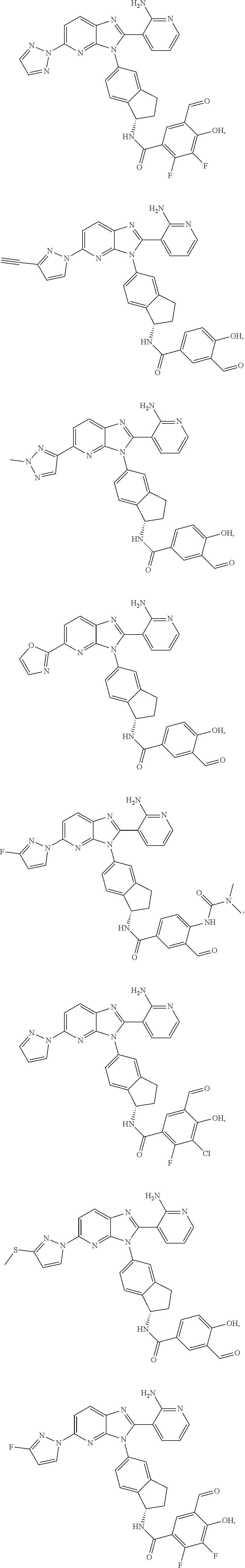 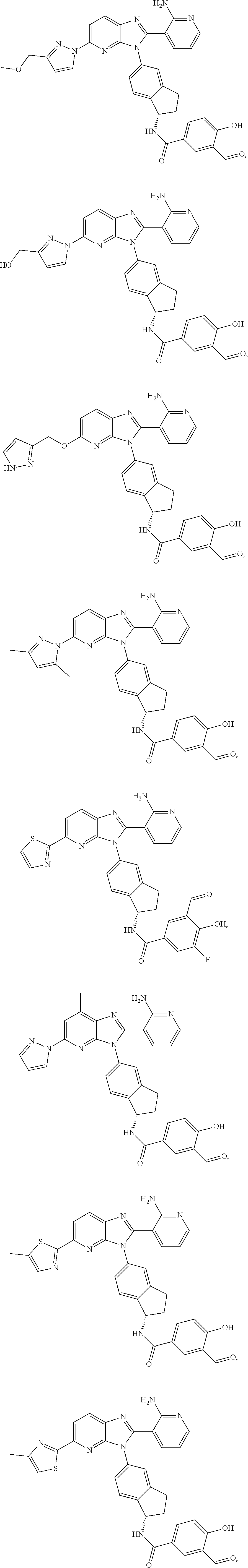 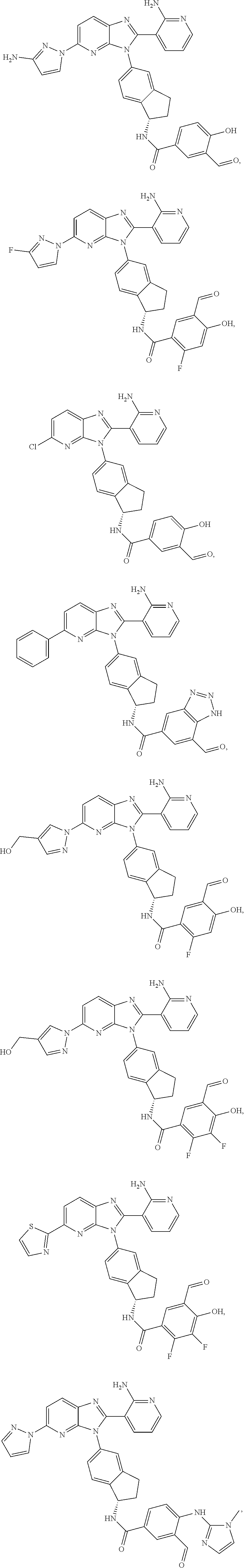 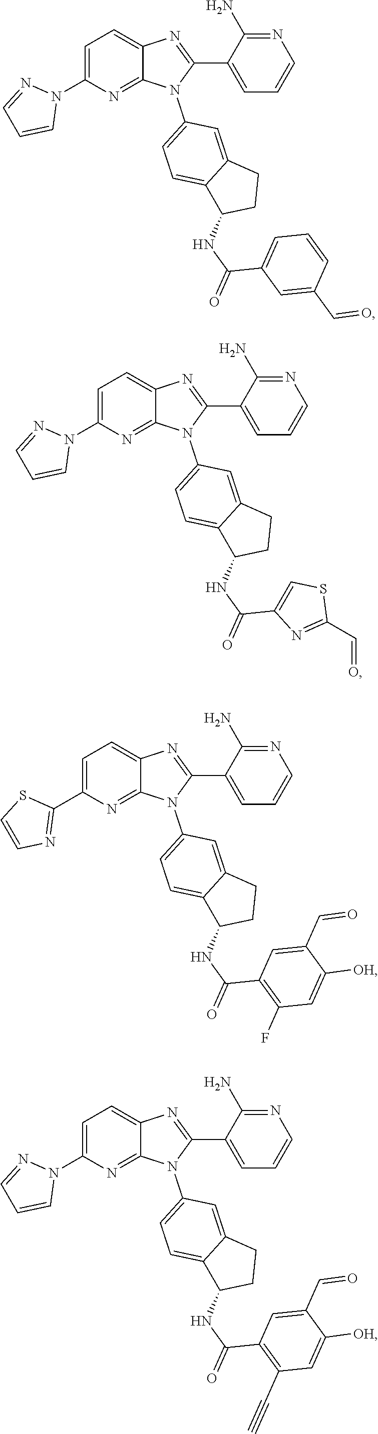 [SMILES: Nc1ncccc1-c1nc2ccc(-n3cccn3)nc2n1-c1ccc2c(c1)CC[C@@H]2NC(=O)c1cccc(C=O)c1 ; Nc1ncccc1-c1nc2ccc(-n3cccn3)nc2n1-c1ccc2c(c1)CC[C@@H]2NC(=O)c1csc(C=O)n1 ; Nc1ncccc1-c1nc2ccc(-c3nccs3)nc2n1-c1ccc2c(c1)CC[C@@H]2NC(=O)c1cc(C=O)c(O)cc1F ; C#Cc1cc(O)c(C=O)cc1C(=O)N[C@H]1CCc2cc(-n3c(-c4cccnc4N)nc4ccc(-n5cccn5)nc43)ccc21] 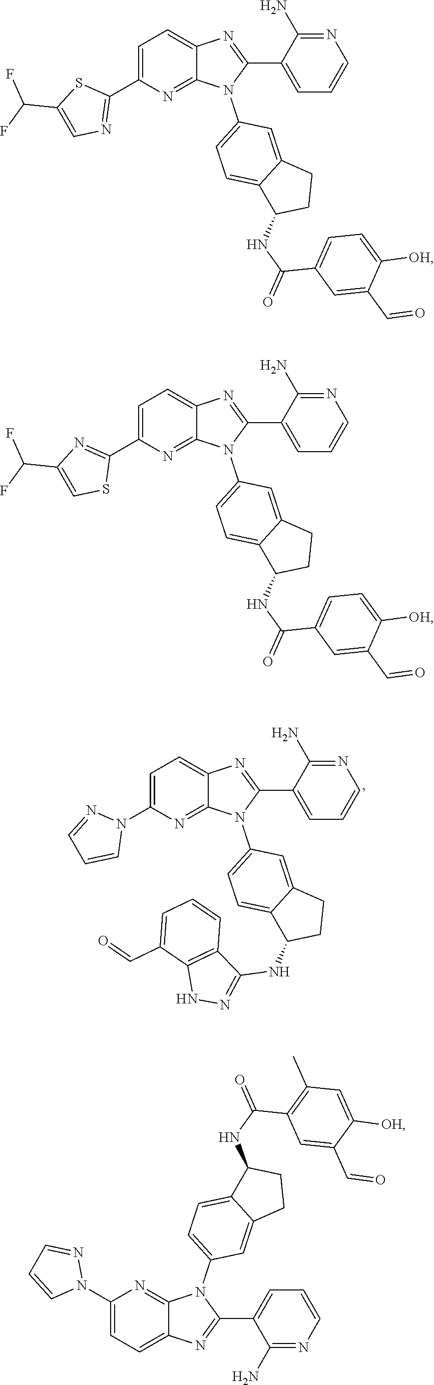 [SMILES: Nc1ncccc1-c1nc2ccc(-c3ncc(C(F)F)s3)nc2n1-c1ccc2c(c1)CC[C@@H]2NC(=O)c1ccc(O)c(C=O)c1 ; Nc1ncccc1-c1nc2ccc(-c3nc(C(F)F)cs3)nc2n1-c1ccc2c(c1)CC[C@@H]2NC(=O)c1ccc(O)c(C=O)c1 ; Nc1ncccc1-c1nc2ccc(-n3cccn3)nc2n1-c1ccc2c(c1)CC[C@@H]2Nc1n[nH]c2c(C=O)cccc12 ; Cc1cc(O)c(C=O)cc1C(=O)N[C@H]1CCc2cc(-n3c(-c4cccnc4N)nc4ccc(-n5cccn5)nc43)ccc21] 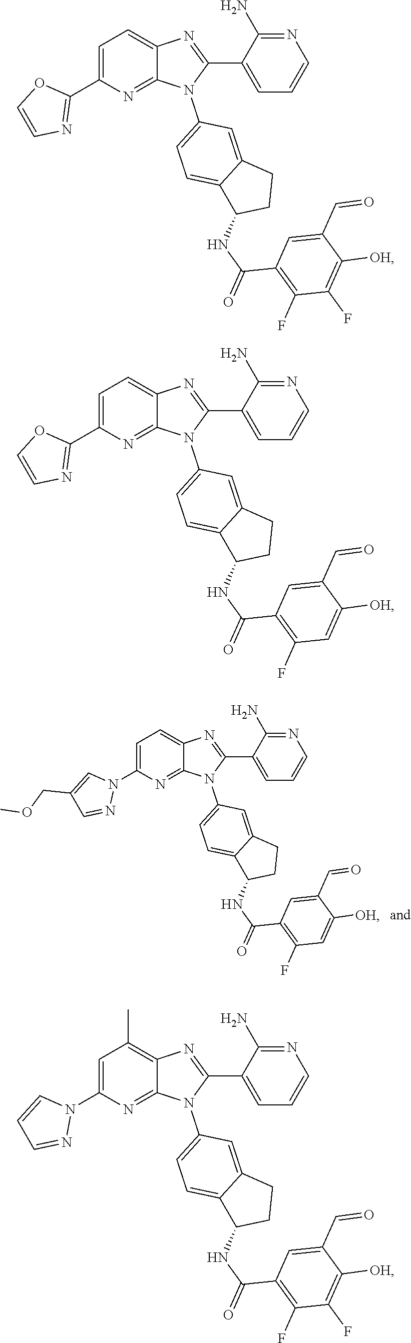 [SMILES: Nc1ncccc1-c1nc2ccc(-c3ncco3)nc2n1-c1ccc2c(c1)CC[C@@H]2NC(=O)c1cc(C=O)c(O)c(F)c1F ; Nc1ncccc1-c1nc2ccc(-c3ncco3)nc2n1-c1ccc2c(c1)CC[C@@H]2NC(=O)c1cc(C=O)c(O)cc1F ; COCc1cnn(-c2ccc3nc(-c4cccnc4N)n(-c4ccc5c(c4)CC[C@@H]5NC(=O)c4cc(C=O)c(O)cc4F)c3n2)c1 ; Cc1cc(-n2cccn2)nc2c1nc(-c1cccnc1N)n2-c1ccc2c(c1)CC[C@@H]2NC(=O)c1cc(C=O)c(O)c(F)c1F]
or a pharmaceutically acceptable salt of any one thereof.

152. A pharmaceutical composition comprising a pharmaceutically acceptable excipient and a compound or salt of claim 1.

153. A method of selectively modulating activity a mutant AKT1 over a wild type AKT comprising administering to a subject in need thereof a compound or salt of claim 1, or a pharmaceutical composition comprising a compound or salt of claim 1 and a pharmaceutically acceptable excipient, wherein the wild type AKT is selected from wild type AKT1 and wild type AKT2.

154. A method of treating cancer in a subject in need thereof, the method comprising administering to the subject a compound or salt of claim 1, or a pharmaceutical composition comprising a compound or salt of claim 1 and a pharmaceutically acceptable excipient.
